(19) United States
(12) Patent Application Publication
(10) Pub. No.: US 2026/0209206 A1
(43) Pub. Date: Jul. 23, 2026

(54) STAT6 DEGRADERS AND USES THEREOF

(71) Applicant: Kymera Therapeutics, Inc., Watertown, MA (US)

(72) Inventors: Bin Yang, Watertown, MA (US); Bruce C. Follows, Watertown, MA (US); Huijun Dong, Watertown, MA (US); Xiao Zhu, Watertown, MA (US); Matthew M. Weiss, Watertown, MA (US); Xiaozhang Zheng, Watertown, MA (US); Xin Huang, Watertown, MA (US); Lijing Su, Watertown, MA (US); Nello Mainolfi, Watertown, MA (US); Thijs Beuming, Cambridge, MA (US); Xue Fei, Watertown, MA (US); Philip Collier, Watertown, MA (US); Yi Zhang, Watertown, MA (US)

(21) Appl. No.: 19/164,725

(22) PCT Filed: Aug. 29, 2024
(86) PCT No.: PCT/US2024/044545

§ 371 Date: Sep. 12, 2025

Related U.S. Application Data
(60) Provisional application No. 63/579,812, filed on Aug. 30, 2023.
(60) Provisional application No. 63/617,359, filed on Jan. 3, 2024.
(60) Provisional application No. 63/649,863, filed on May 20, 2024.
(60) Provisional application No. 63/678,903, filed on Aug. 2, 2024.

(51) Int. Cl. C07D 401/14; A61K 31/4545; A61K 31/55; C07D 405/14; C07D 413/14

(52) U.S. Cl. CPC C07D 401/14; A61K 31/4545; A61K 31/55; C07D 405/14; C07D 413/14

(57) ABSTRACT

The present invention provides compounds, compositions thereof, and methods of using the same.

### CROSS REFERENCE TO RELATED APPLICATIONS

This application claims the benefit of priority from U.S. Provisional Appl. No. 63/579,812, filed Aug. 30, 2023, U.S. Provisional Appl. No. 63/617,359, filed Jan. 3, 2024, U.S. Provisional Appl. No. 63/649,863, filed May 20, 2024, and U.S. Provisional Appl. No. 63/678,903, filed Aug. 2, 2024.

### TECHNICAL FIELD OF THE INVENTION

The present invention relates to compounds and methods useful for the modulation of signal transducer and activator of transcription 6 (“STAT6”) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews, C., Chemistry & Biology, 2010, 17(6):551-555: Schnnekloth, J. S. Jr., Chembiochem, 2005, 6(1):40-46).

Signal transducer and activator of transcription 6 (STAT6 or Interleukin-4-Stat/IL4-STAT) is an undruggable transcription factor belonging to the structurally conserved Signal Transducer and Activator of Transcription (STAT) family of proteins (STAT1 through STAT6). Activation of STAT6, like other STAT proteins, is triggered upon binding of hormones, immunomodulatory cytokines or growth factors to specific receptors on the cell surface. Once activated, the phosphorylation of a C-terminal tyrosine residue occurs, leading to translocation and transmission of signals from the cytosol to the nucleus, resulting in activation of gene expression.

STAT6 is implicated in driving Type 2 immunity, allergies. It may participate in IL-4/IL-13-mediated allergic reaction, and play a vital role in the differentiation of T-helper type 2 (Th2) cells (Hebenstreit et al. “Signaling mechanisms, interaction partners, and target genes of STAT6.” Cytokine & growth factor reviews 17.3 (2006): 173-188; Chapoval et al. “Regulation of the T helper cell type 2 (Th2)/T regulatory cell (Treg) balance by IL-4 and STAT6.” Journal of leukocyte biology 87.6 (2010): 1011-1018). STAT6 is a key node primarily activated in the Janus Kinase (JAK) pathway by inflammatory cytokines, interleukin-4 (IL4) and interleukin-13 (IL13) and their cognate receptors, which are produced by Th2 cells, mast cells and basophils. Human STAT6 mutations have been associated with severe allergies such as asthma and eczema (Goenka and Kaplan. “Transcriptional regulation by STAT6.” Immunologic research 50.1 (2011): 87-96). There is a need to discover and develop STAT6 drugs, for example to treat allergic/inflammatory diseases and cancers (Glosson et al. “Wheezing and itching: The requirement for STAT proteins in allergic inflammation.” Jak-Stat 1.1 (2012): 3-15: Loh et al. “Signal transducer and activator of transcription (STATs) proteins in cancer and inflammation: functions and therapeutic implication.” Frontiers in oncology 9 (2019): 48). As such, small molecule compounds that leverage E3 ligase mediated protein degradation to target disease-associated proteins such as STAT6 hold promise as therapeutic agents.

### SUMMARY OF THE INVENTION

The present application relates to novel bifunctional compounds, which function to recruit STAT6 protein to E3 ubiquitin ligase for degradation, and methods of preparation and uses thereof. In particular, the present disclosure provides bifunctional compounds, which find utility as modulators of targeted ubiquitination of STAT6 protein, which are then degraded and/or otherwise inhibited by the bifunctional compounds as described herein. Also provided are monovalent compounds, which find utility as inducers of targeted ubiquitination of STAT6 protein, which are then degraded and/or otherwise inhibited by the monovalent compounds as described herein. An advantage of the compounds provided herein is that a broad range of pharmacological activities is possible, consistent with the degradation/inhibition of STAT6 protein. In addition, the description provides methods of using an effective amount of the compounds as described herein for the treatment or amelioration of a disease condition, such as inflammatory disorders.

The present application further relates to targeted degradation of STAT6 protein through the use of bifunctional molecules, including bifunctional molecules that link a cereblon or VHL binding moiety to a ligand that binds STAT6 protein.

It has now been found that compounds of this invention, and pharmaceutically acceptable compositions thereof, are effective as degraders of STAT6 protein. Such compounds have the general formula I:

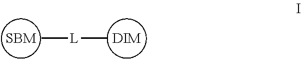

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating STAT6 protein. Such diseases, disorders, or conditions include those described herein.

Compounds provided by this invention are also useful for the study of STAT6 protein in biological and pathological phenomena: the study of intracellular signal transduction pathways occurring in bodily tissues; and the comparative evaluation of new STAT6 inhibitors or STAT6 degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of STAT6 protein. In some embodiments, a provided compound degrades and/or inhibits STAT6.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B, and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

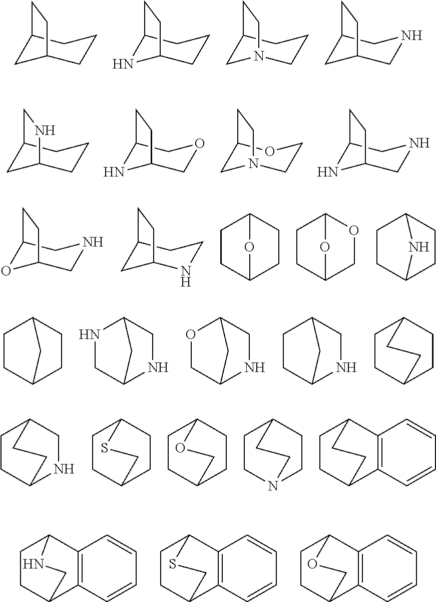

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon: the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl)).

The term “unsaturated.” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

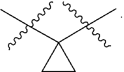 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylenyl” refers to bivalent aryl groups (e.g., phenylenyl).

The terms “heteroaryl” and “heteroar-,” used alone or as part of a larger moiety, e.g. “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, bridged bicyclic, or spirocyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “heteroarylenyl” refers to bivalent heteroaryl groups (e.g., pyridylenyl).

As used herein, the terms “heterocycle,” “heterocyclyl,” “heterocyclic radical.” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ′NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁₀—P which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄₀C(O)R^{o}; —OC(O)(CH₂)₀₋₄S R^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂. —N(R^{o})S(O)₂R^{o}; —N(OR^{o})R^{o}; —C(NH)NR^{o}₂; —(CH₂)₁₋₄P(O)₂R^{o}; —(CH₂)₀₋₄P(O)R^{o}₂; —(CH₂)₀₋₄OP(O)R^{o}₂; —(CH₂)₀₋₄OP(O)(OR^{o})₂: SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR—, —(CH₂)₀₋₂SH, —(CH₂)₀₋₂NH₂, —(CH₂)₀₋₂NHR^{•}, —(CH₂)₀₋₂NR₂, —NO₂, —SiR₃, —OSiR^{o}₃, —C(O)SR, —(C₁₋₁₀ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁, Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include; —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR*, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}: wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rf are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromatographic purification are contemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the term “provided compound” refers to any genus, subgenus, and/or species set forth herein.

As used herein, the term “inhibitor” is defined as a compound that binds to and/or inhibits STAT6 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both STAT6 protein and an E3 ligase with measurable affinity resulting in the ubiquitination and subsequent degradation of the STAT6 protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed, 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R⁶G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow. Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in STAT6 protein activity between a sample comprising a compound of the present invention, or composition thereof, and STAT6 protein, and an equivalent sample comprising STAT6 protein, in the absence of said compound, or composition thereof.

As used herein, the term “reference” describes a standard or control relative to which a comparison is performed. In some embodiments, a “reference” sample or subject is one that is sufficiently similar to a particular sample or subject of interest to permit a relevant comparison. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

### 3a. Description of Exemplary Embodiments Related to Methods of Use

In certain embodiments, the present invention provides methods of use related to a compound of formula I:

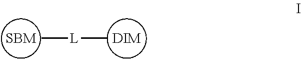

or a pharmaceutically acceptable salt thereof, wherein:
SBM is a STAT6 binding moiety capable of binding to STAT6 protein as described within this section (3a) below and herein;
L is a bivalent moiety that connects SBM to DIM as described within this section (3a) below and herein; and
DIM is a degradation inducing moiety selected from an E3 ubiquitin ligase binding moiety (LBM), lysine mimetic, and hydrogen, as described within this section (3a) below and herein.

In some embodiments, the compound of formula I specifically affects, as its primary mechanism of action, the degradation of STAT6.

It will be understood that formula I cannot extend beyond the formulae described within this section (3a) below and herein. It will be understood that all references to “defined and described herein” solely refer to this section (3a) of the application. Additionally, all chemical formulae and embodiments within section 3a can only apply to and can be combined with formulae and embodiments from this section (3a).

### STAT6 Binding Moiety (SBM)

In some embodiments, the present invention provides methods of degrading STAT6, or a mutant thereof, or methods of treating a STAT6-mediated disease, disorder, or condition, comprising administering compound of formula I-a″″:

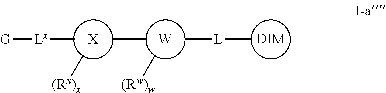

or a pharmaceutically acceptable salt thereof, wherein L and DIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

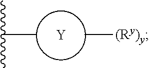

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B′} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and y are independently 0, 1, 2, 3, or 4;
with the proviso that the compound or pharmaceutically acceptable salt thereof is other than a compound of formula I-a″″-1:

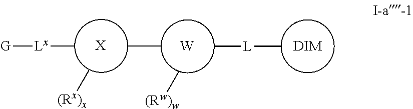

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5′, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, 1-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2′, I-ccc-3′, I-ccc′-1′, I-ccc″-1′, I-ccc-A, I-ccc-B, I-ccc-C, I-ddd, I-ff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
   G is hydrogen, halogen, or

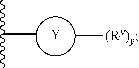

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

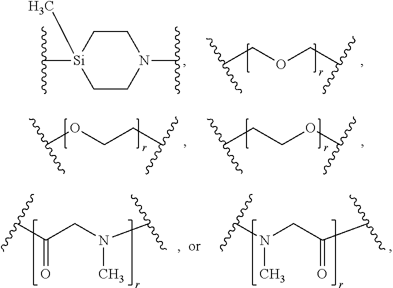

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10;
   or
(ii)

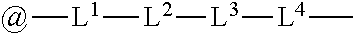

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—:
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-, Cy^{L2}-, -Cy^{L3}- and -Cy^{L4}- is independently -Cy-.

In some embodiments, the present invention provides methods of degrading STAT6, or a mutant thereof, or methods of treating a STAT6-mediated disease, disorder, or condition, comprising administering a compound of formula I-a″″:

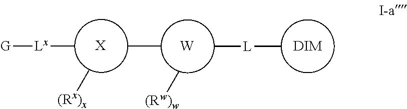

or a pharmaceutically acceptable salt thereof, wherein L and DIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-8 or 10-14 membered monocyclic, bicyclic, or tricyclic heteroaryl with 1-4 heteroatoms independently selected from nitrogen, oxygen and sulfur;
Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

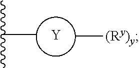

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-8 or 10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-8 or 10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B′} is independently L^{B}Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-8 or 10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-8 or 10 membered monocyclic or bicyclic aryl or heteroaryl with 1-4 heteroatoms independently selected from nitrogen, oxygen and sulfur;
each R is independently hydrogen, or a group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-8 or 10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is a ring selected from phenyl, a 3-7 membered saturated or partially unsaturated carbocyclylenyl or heterocyclylenyl having 1-4 heteroatoms independently selected from nitrogen, oxygen, and sulfur, and a 5-8 membered monocyclic or bicyclic heteroarylenyl having 1-4 heteroatoms independently selected from nitrogen, oxygen, and sulfur; and
each of w, x, and y are independently 0, 1, 2, 3, or 4.

In some embodiments, the present invention provides methods of degrading STAT6, or a mutant thereof, or methods of treating a STAT6-mediated disease, disorder, or condition, comprising administering a compound of formula I-a″″:

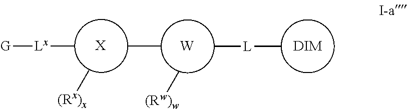

or a pharmaceutically acceptable salt thereof, wherein L and DIM are as defined and described herein, wherein:
Ring W is a ring selected from phenyl, naphthyl, 5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-5 or 7-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

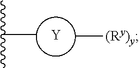

Ring Y is a ring selected from phenyl, naphthyl, 3-5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
wherein R^{y} and R^{x} are not indolyl or azaindolyl;
each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₅ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur,
each R^{B′} is independently -L^{B}-Cy^{B1}-H or -L^{B}-Cy^{B1}-Cy^{B2};
each L^{B} is independently a covalent bond or a C₁₋₆ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-5 or 7-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-5 or 7-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or a group selected from C₁₋₅ aliphatic, phenyl, a 4-5 or 7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-5 or 7-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—:
each -Cy^{x}- is a ring selected from phenyl, a 3-5 or 7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and v are independently 0, 1, 2, 3, or 4.

It will be understood that, throughout the entirety of this disclosure including all embodiments and formulae within section 3a alone or in combination, the described compounds or pharmaceutically acceptable salts thereof are other than a compound of formula I-a″″-1:

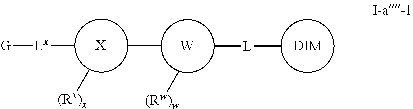

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, I-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2, I-ccc-3′, I-ccc′-1′, I-ccc″-1′, I-ccc-A, I-ccc-B, I-ccc-C, I-ddd, I-fff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
   G is hydrogen, halogen, or

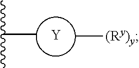

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₂(hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

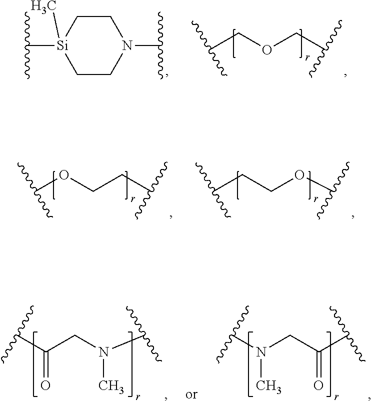

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10;
   or
(ii)

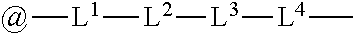

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—: and
each of -Cy^{L1}-Cy^{L2}, -Cy^{L3}-, and -Cy^{L4}- is independently -Cy-.

As described above and defined herein, Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-14 membered monocyclic, bicyclic, or tricyclic heteroaryl with 1-4 heteroatoms independently selected from nitrogen, oxygen, and sulfur.

In some embodiments, Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-13 membered monocyclic, bicyclic, or tricyclic heteroaryl with 1-4 heteroatoms independently selected from nitrogen, oxygen, and sulfur.

In some embodiments, Ring W is phenyl. In some embodiments, Ring W is naphthyl. In some embodiments, Ring W is a 5-11 membered saturated or partially unsaturated monocyclic, bicyclic, bridged bicyclic, or spirocyclic carbocyclyl. In some embodiments, Ring W is a 5-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring W is a 5-13 membered monocyclic, bicyclic, or tricyclic heteroaryl with 1-4 heteroatoms independently selected from nitrogen, oxygen, and sulfur. In some embodiments, Ring W is a 5-10 membered monocyclic or bicyclic heteroaryl with 1-4 heteroatoms independently selected from nitrogen, oxygen, and sulfur.

In some embodiments, Ring W is a 8-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 8-13 membered bicyclic heteroaryl with 1-4 heteroatoms independently selected from nitrogen, oxygen, and sulfur.

In some embodiments, Ring W is an 8-11 membered saturated or partially unsaturated bicyclic heterocyclyl with 1-3 heteroatoms independently selected from nitrogen, oxygen, and sulfur. In some embodiments, Ring W is a 9-10 membered saturated or partially unsaturated bicyclic heterocyclyl with 1-3 heteroatoms independently selected from nitrogen, oxygen, and sulfur. In some embodiments, Ring W is a 9 membered saturated or partially unsaturated bicyclic heterocyclyl with 1-3 heteroatoms independently selected from nitrogen, oxygen, and sulfur. In some embodiments, Ring W is isisoindolinyl, isoindolinonyl, isoindolinedionyl, pyrrolopyridinyl, pyrrolopyrimidinyl, 6,7-dihydro-5H-pyrrolo[3,4]-d]pyrimidinyl, tetrahydropyrrolo[3,2-c]pyridinyl, tetrahydroindolyl, quinolizinyl, or tetrahydropyrazolo[1,5-a]pyrimidinyl.

In some embodiments, Ring W is a 10 membered saturated or partially unsaturated bicyclic heterocyclyl with 1-3 heteroatoms independently selected from nitrogen, oxygen, and sulfur. In some embodiments, Ring W is chromanyl, chromenyl, isochromenyl, tetrahydroisoquinolinyl, tetrahydroquinolinyl, or 1,2-dihydroquinolinyl, 1,2-dihydroisoquinolinyl.

Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;

In some embodiments, Ring W is a 5-8 membered saturated or partially unsaturated monocyclic heterocyclyl with 1-4 heteroatoms independently selected from nitrogen, oxygen, and sulfur. In some embodiments, Ring W is a 5-8 membered monocyclic heteroaryl with 1-4 heteroatoms independently selected from nitrogen, oxygen, and sulfur. In some embodiments, Ring W is a 9-10 membered saturated or partially unsaturated bicyclic heterocyclyl with 1-4 heteroatoms independently selected from nitrogen, oxygen, and sulfur. In some embodiments, Ring W is a 9-10 membered heteroaryl with 1-4 heteroatoms independently selected from nitrogen, oxygen, and sulfur. In some embodiments, Ring W is an 11-14-membered tricyclic heteroaryl with 1-4 heteroatoms independently selected from nitrogen, oxygen, and sulfur.

In some embodiments, Ring W is a 5-6 membered heteroaryl with 1-4 heteroatoms independently selected from nitrogen, oxygen, and sulfur. In some embodiments, Ring W is a 5 membered heteroaryl with 1-4 heteroatoms independently selected from nitrogen, oxygen, and sulfur. In some embodiments, Ring W is furanyl, oxazolyl, isoxazolyl, or oxadiazolyl. In some embodiments, Ring W is a 6 membered heteroaryl with 1-4 heteroatoms independently selected from nitrogen, oxygen, and sulfur. In some embodiments, Ring W is pyridinyl, pyrimidinonyl, pyridazinyl, or triazinyl.

In some embodiments, Ring W is a 9-membered bicyclic heteroaryl with 1-4 heteroatoms independently selected from nitrogen, oxygen, and sulfur. In some embodiments, Ring W is a 9-membered bicyclic heteroaryl with 1-2 heteroatoms independently selected from nitrogen, oxygen, and sulfur.

In some embodiments, Ring W is a 9-membered bicyclic heteroaryl with 1 heteroatom independently selected from nitrogen, oxygen, and sulfur. In some embodiments, Ring W is a 9-membered bicyclic heteroaryl with 2 heteroatoms independently selected from nitrogen, oxygen, and sulfur. In some embodiments, Ring W is indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl.

In some embodiments, Ring W is azaindazolyl (e.g., 4-, 5-, 6-, or 7-azaindazolyl). In some embodiments, Ring W is pyrrolol[2,3-c]pyridinyl. In some embodiments, Ring W is indolizinyl.

In some embodiments, Ring W is a 10-membered bicyclic heteroaryl with 1-4 heteroatoms independently selected from nitrogen, oxygen, and sulfur. In some embodiments, Ring W is a 10-membered bicyclic heteroaryl with 1-2 heteroatoms independently selected from nitrogen, oxygen, and sulfur. In some embodiments, Ring W is a 10-membered bicyclic heteroaryl with 1-2 nitrogen heteroatoms. In some embodiments, Ring W is quinolinyl, isoquinolinyl, quinolizinyl, quinoxalinyl, phthalazinyl, quinazolinyl, cinnolinyl, or 1,8-naphthyridinyl.

In some embodiments, Ring W is a 10-14 membered tricyclic heteroaryl with 1-4 heteroatoms independently selected form nitrogen, oxygen, and sulfur. In some embodiments, Ring W is a 13 membered tricyclic heteroaryl with 1-4 heteroatoms independently selected form nitrogen, oxygen, and sulfur. In some embodiments, Ring W is:

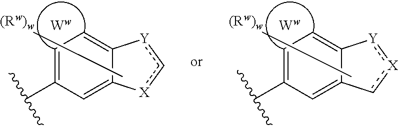

wherein X, Y, R^{w}, and w are as defined above and described herein; and
Ring W^{W} is a fused phenyl or 5-6 membered saturated or partially unsaturated carbocyclyl or heterocyclyl having 1-2 heteroatoms independently selected from nitrogen, oxygen, and sulfur, or a 5-6 membered heteroaryl having 1-2 heteroatoms independently selected from nitrogen, oxygen, and sulfur.

As defined above and described herein, Ring W^{W} is a fused phenyl or 5-6 membered saturated or partially unsaturated carbocyclyl or heterocyclyl having 1-2 heteroatoms independently selected from nitrogen, oxygen, and sulfur, or a 5-6 membered heteroaryl having 1-2 heteroatoms independently selected from nitrogen, oxygen, and sulfur. In some embodiments, Ring W^{W} is a fused phenyl. In some embodiments, Ring W^{W} is a 5-6 membered saturated or partially unsaturated carbocyclyl. In some embodiments, Ring W^{W} is a 5-6 membered saturated or partially unsaturated heterocyclyl having 1-2 heteroatoms independently selected from nitrogen, oxygen, and sulfur. In some embodiments, Ring W^{W} is a 5-6 membered heteroaryl having 1-2 heteroatoms independently selected from nitrogen, oxygen, and sulfur. In some embodiments, Ring W is

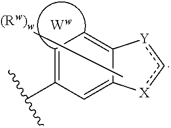

It will be understood that, in the preceding or following paragraphs where only a single point of attachment

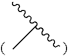

at Ring W is depicted, the depicted

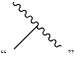

represents the point of attachment to Ring X, and an additional point of attachment to -L-DIM may replace an —H at any point on Ring W as allowed by valency.

In some embodiments, Ring W is

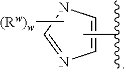

In some embodiments, Ring W is

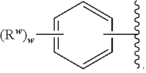

In some embodiments, Ring W is

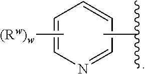

In some embodiments, Ring W is

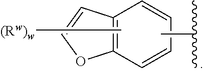

In some embodiments, Ring W is

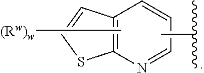

In some embodiments, Ring W is

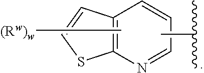

In some embodiments, Ring W is

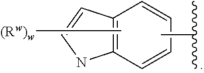

In some embodiments, Ring W is

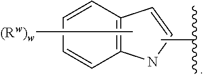

In some embodiments, Ring W is

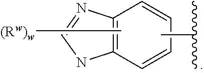

In some embodiments, Ring W is

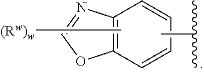

N In some embodiments, Ring W is

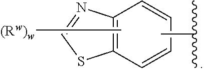

In some embodiments, Ring W is

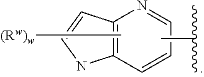

In some embodiments, Ring W is

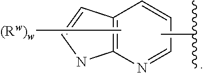

In some embodiments, Ring W is

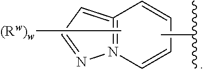

In some embodiments, Ring W is

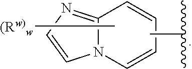

In some embodiments, Ring W is

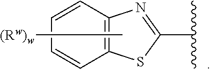

In some embodiments, Ring W is

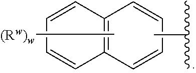

In some embodiments, Ring W is

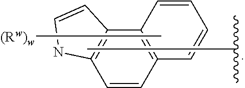

In some embodiments, Ring W is

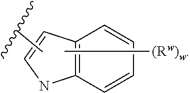

In some embodiments, Ring W is

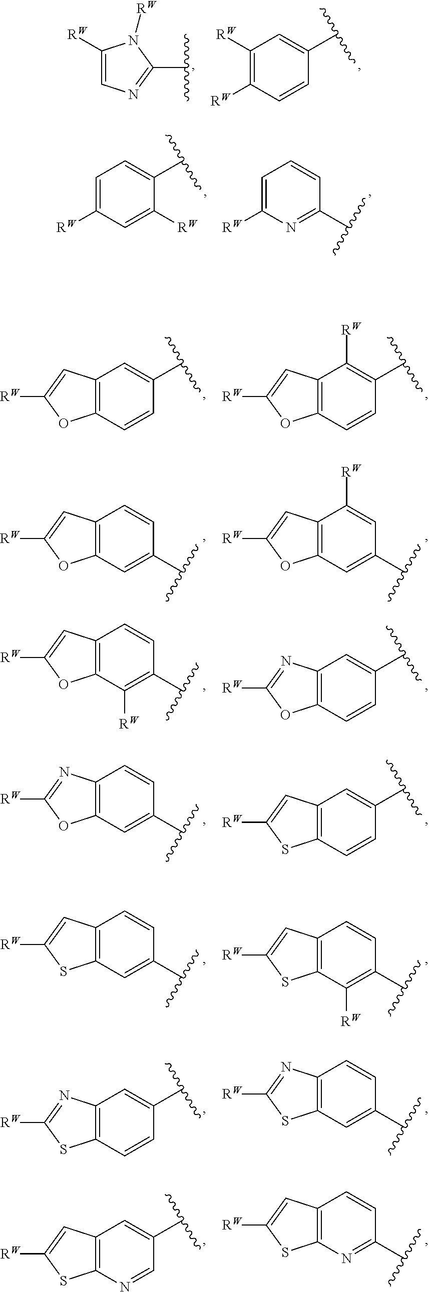

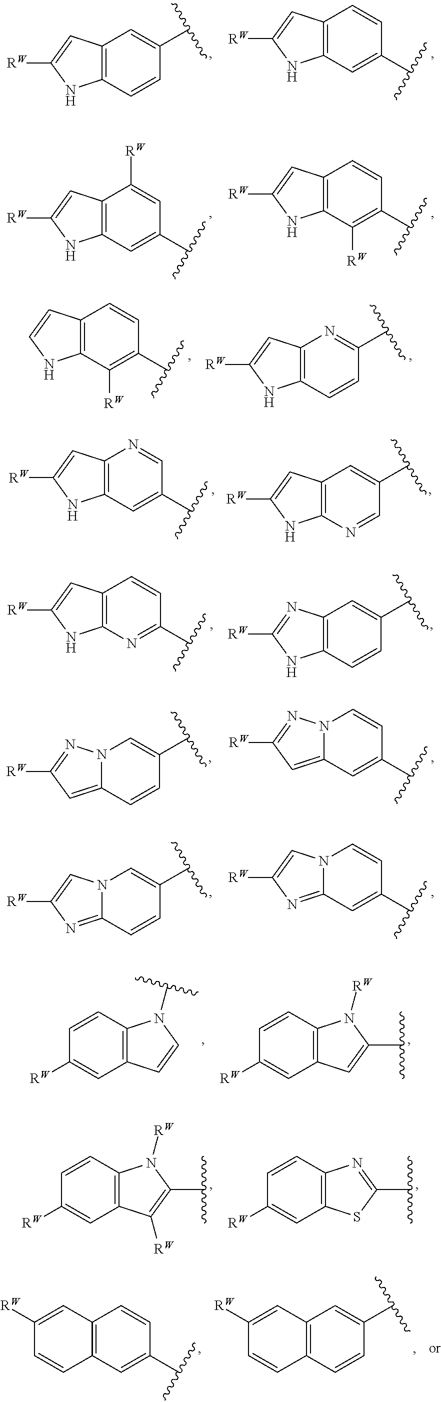

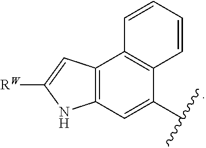

In some embodiment, Ring W is as depicted in the compounds of Table 1A, below.

In some embodiments, Ring W is

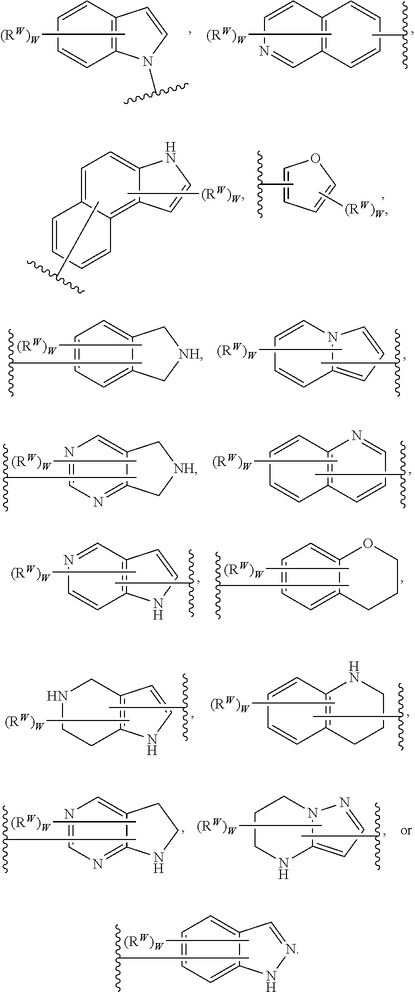

In some embodiments, Ring W is

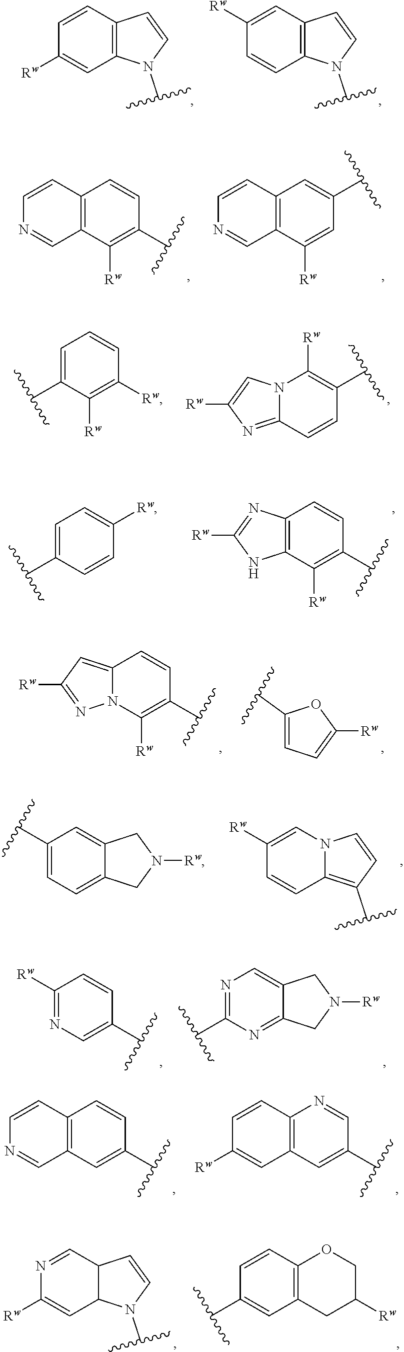

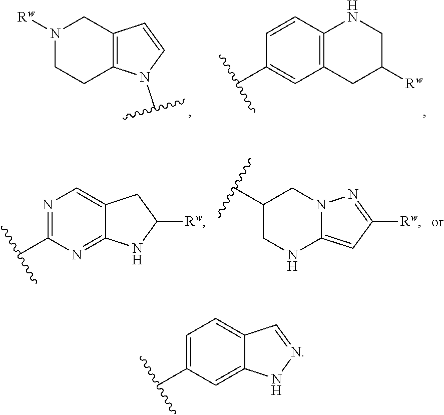

In some embodiments, Ring W is

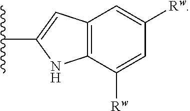

In some embodiments, Ring W is

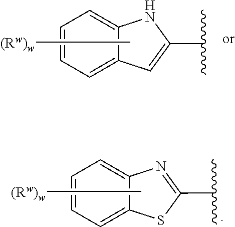

In some embodiments, Ring W is

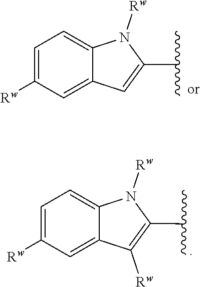

In some embodiments, Ring W is

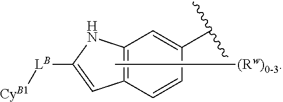

In some embodiments, Ring W is

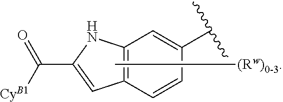

In some embodiments, Ring W and its R^{w} substituents are

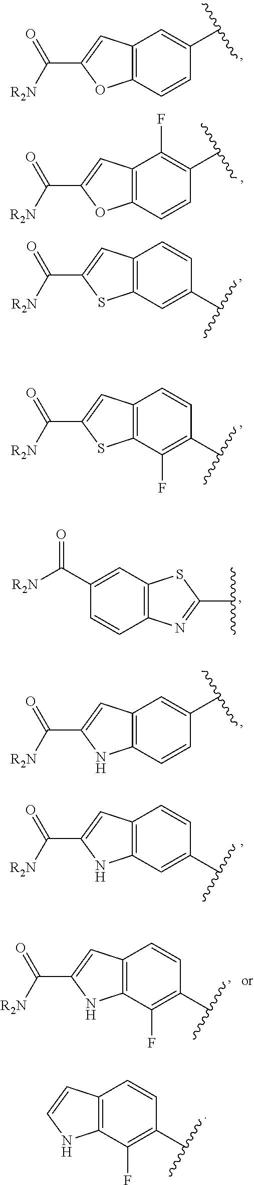

In some embodiments, Ring W and its R^{w} substituents are

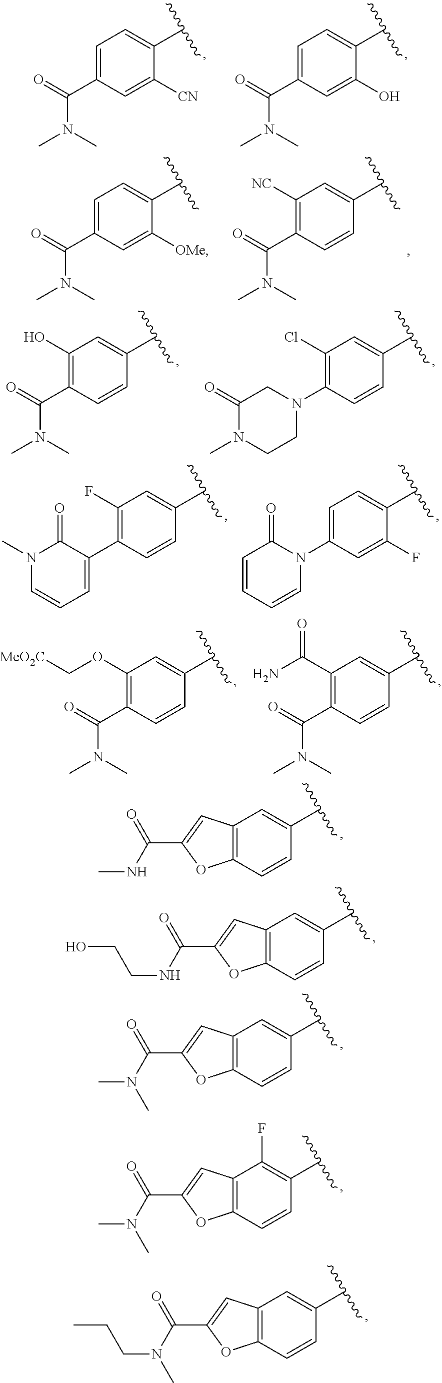

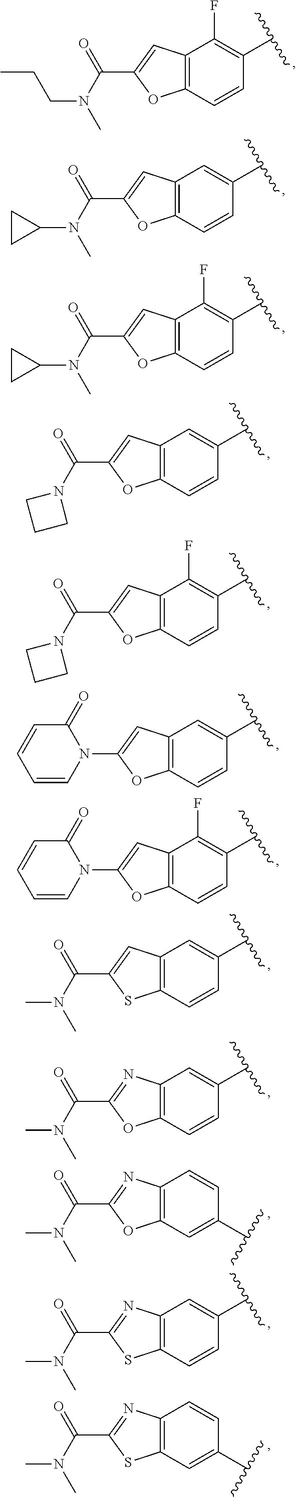

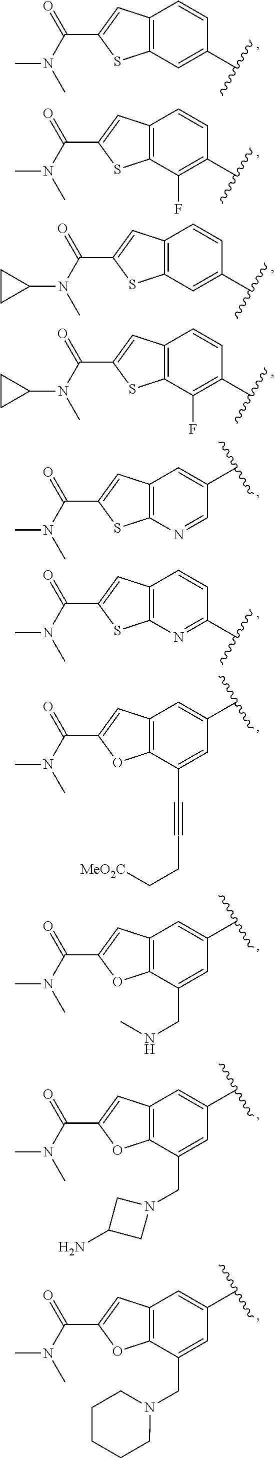

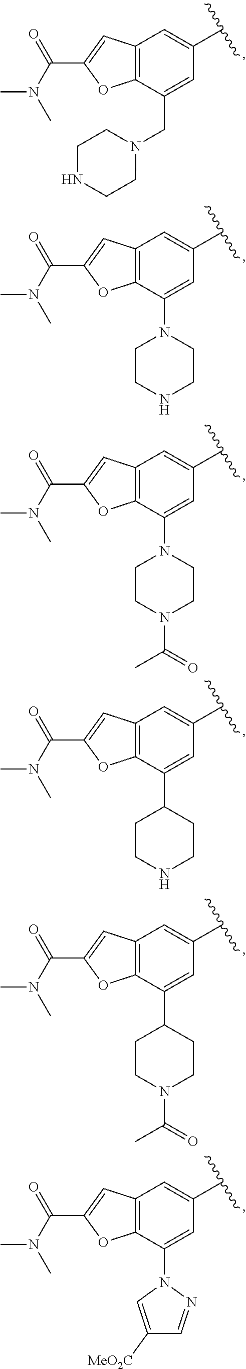

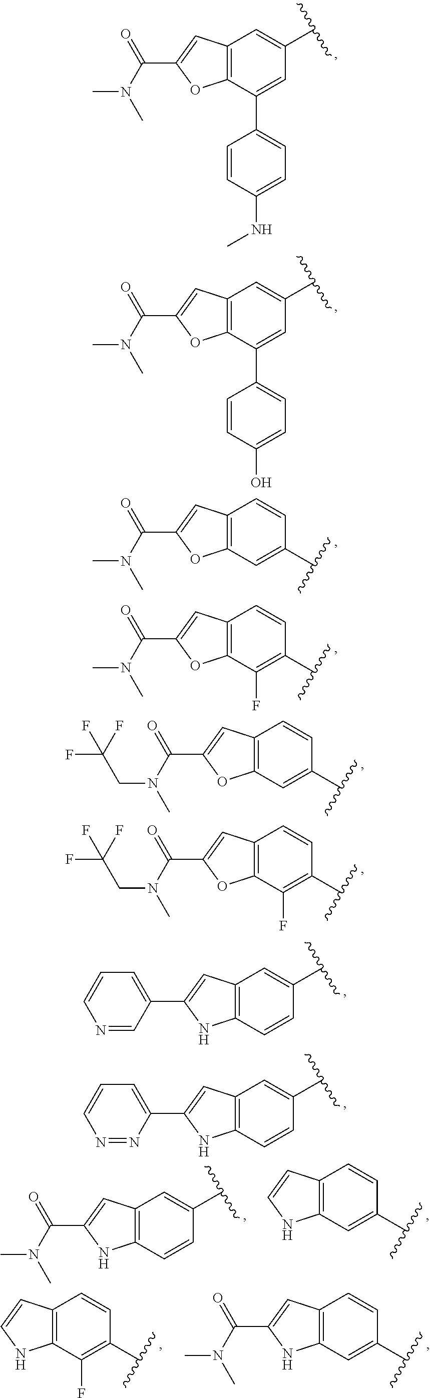

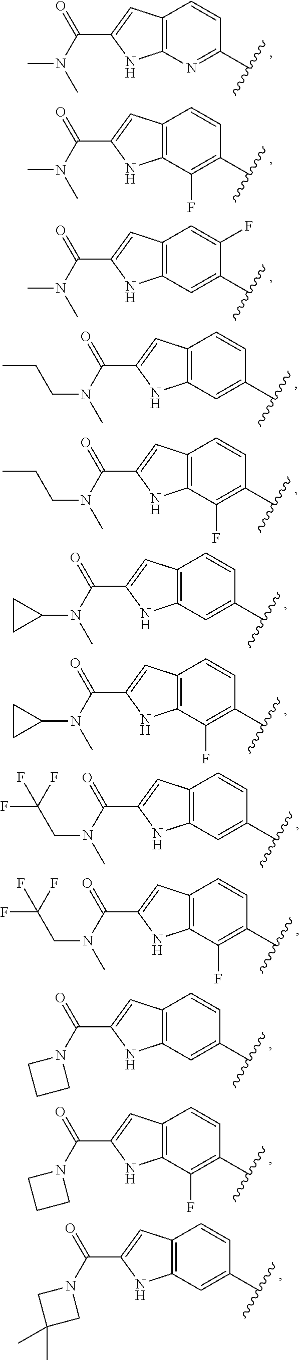

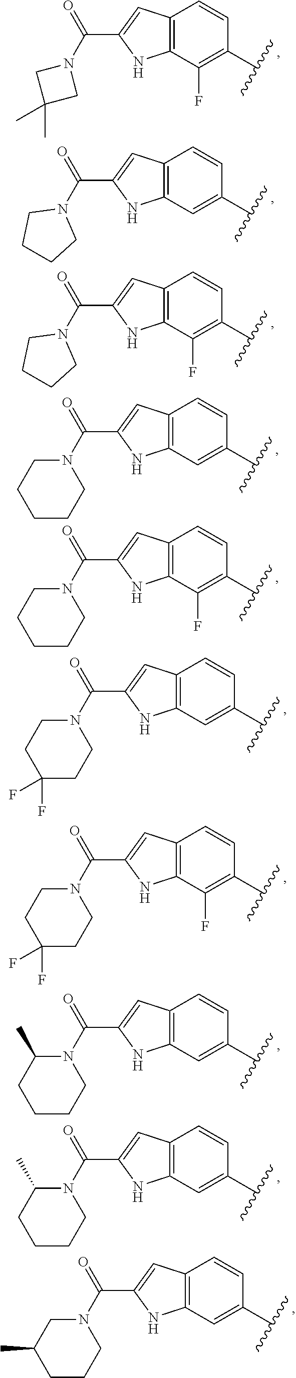

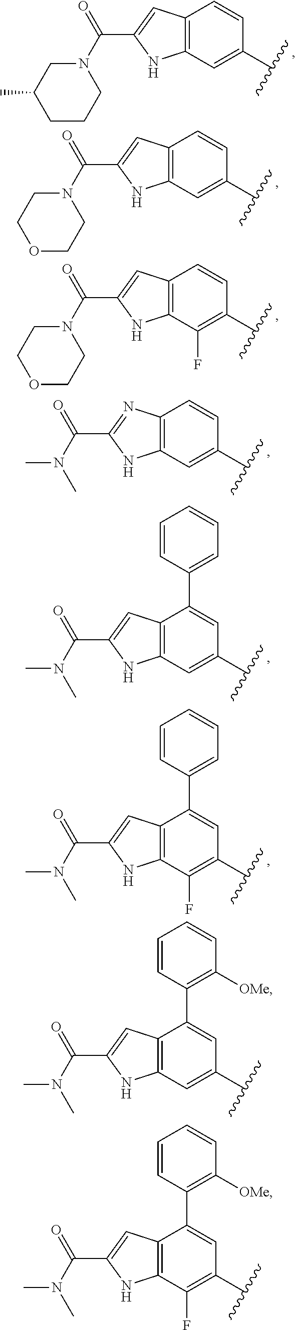

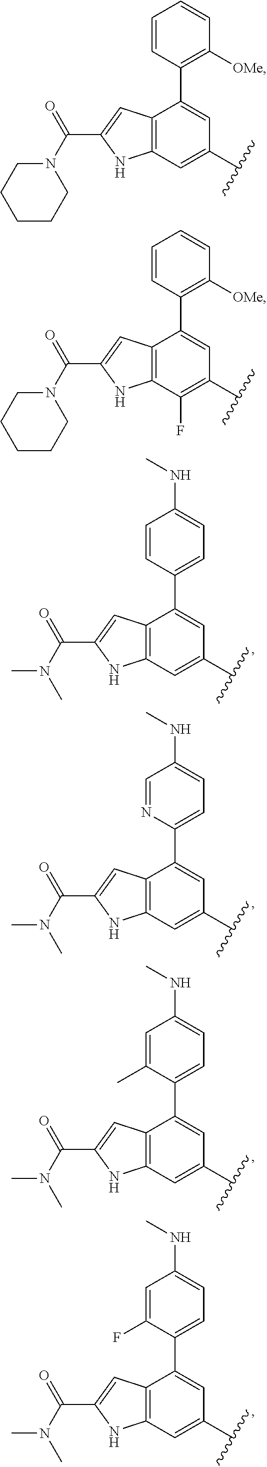

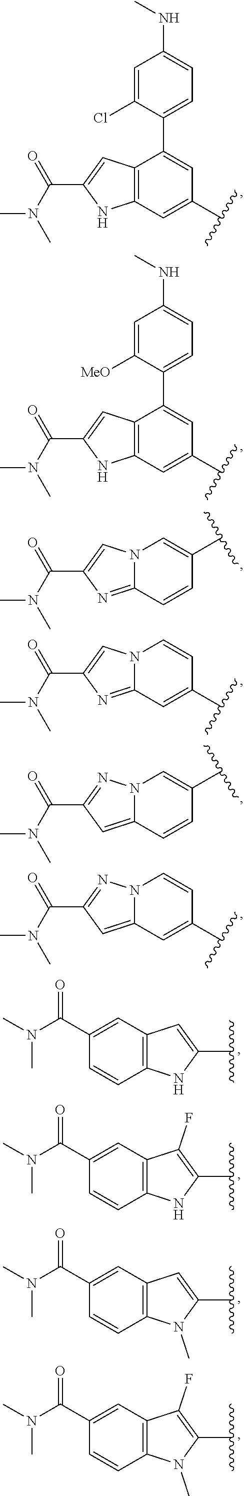

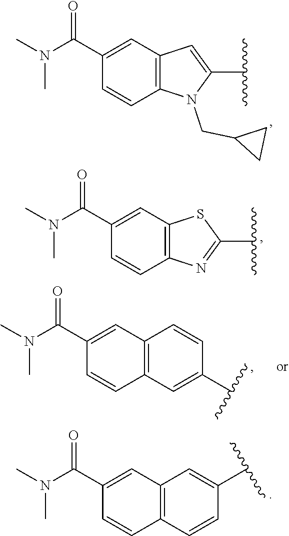

In some embodiments, Ring W and its R^{W} substituents are

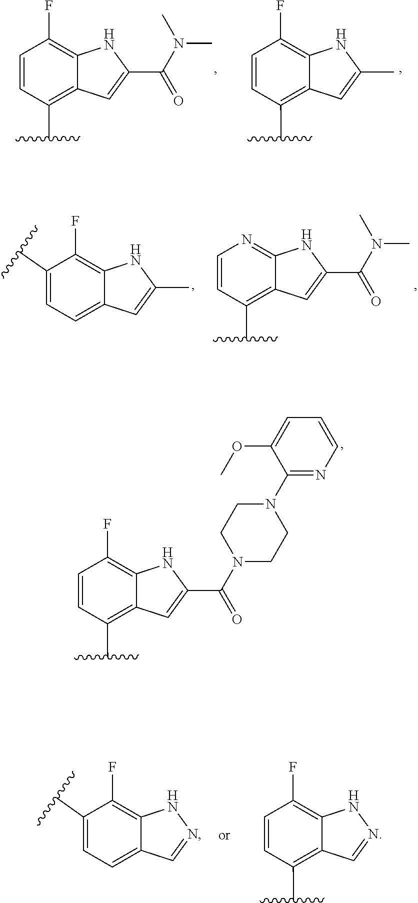

In some embodiments, Ring W and its R^{W} substituents are

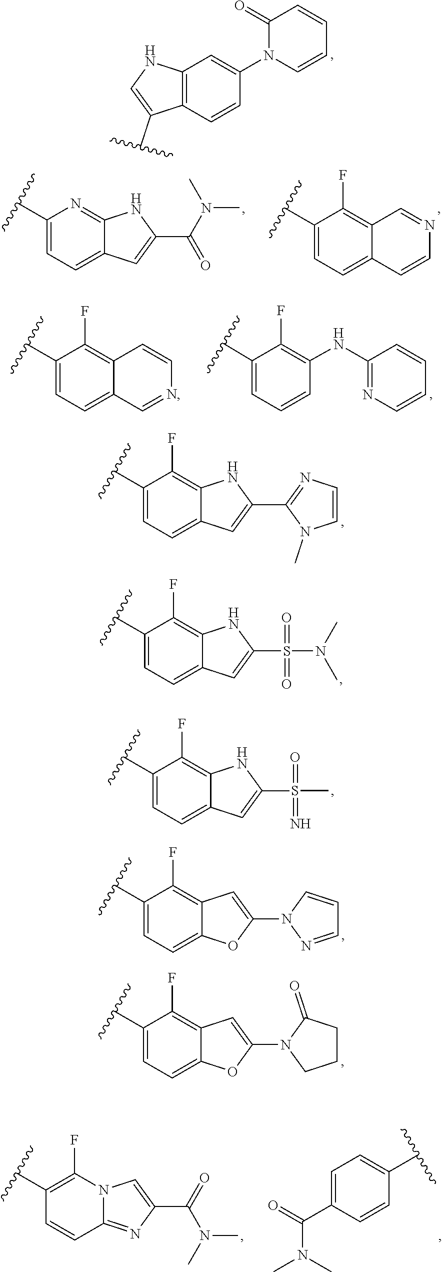

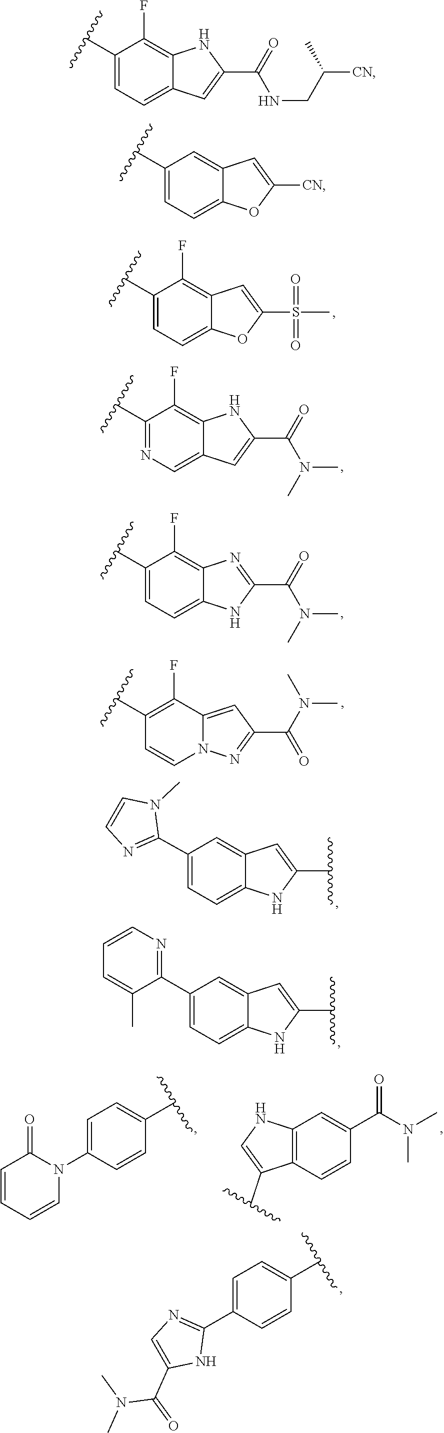

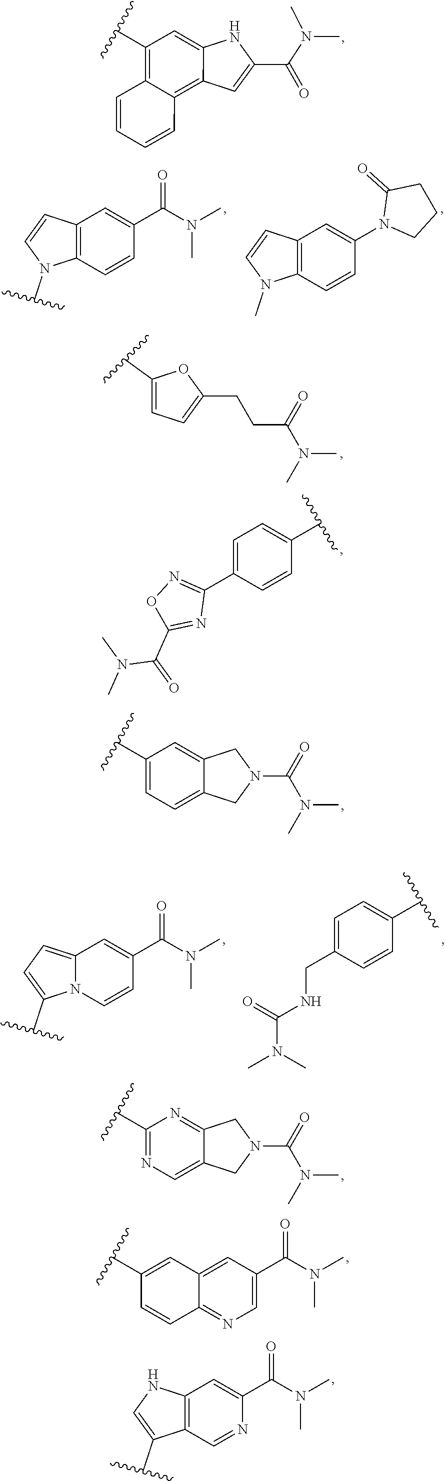

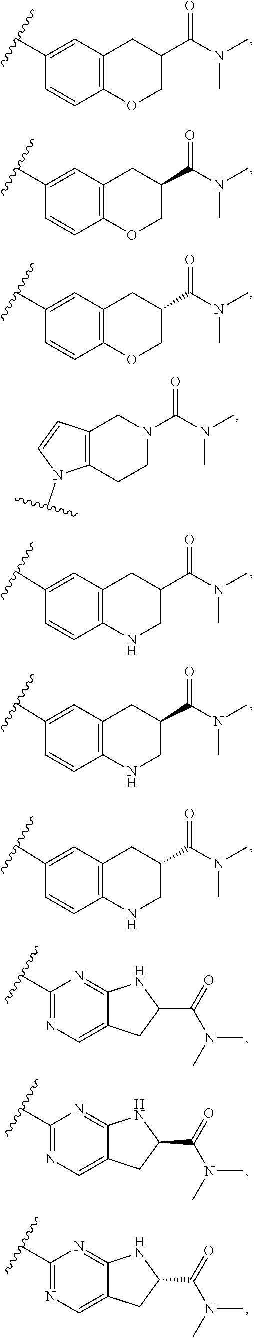

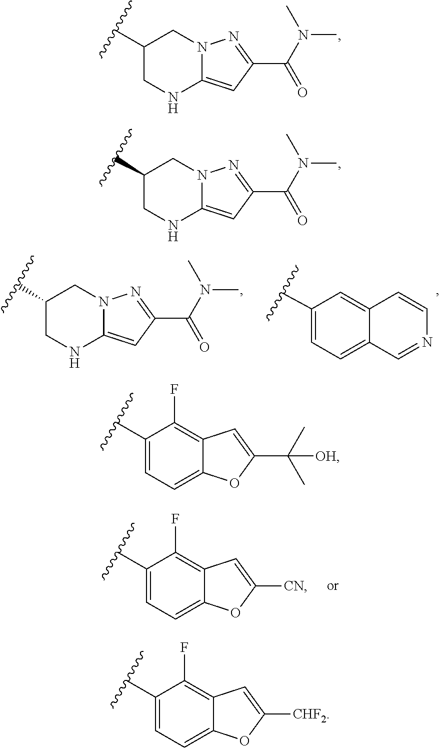

In some embodiments, Ring W and its R^{W} substituents are

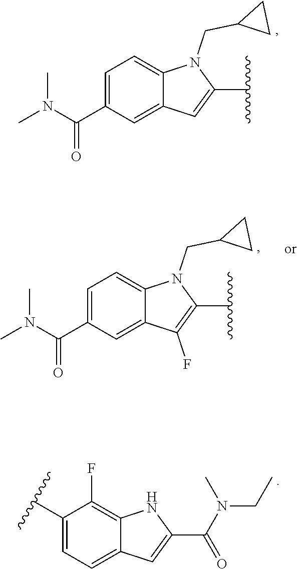 [SMILES: *c1cc2cc(C(=O)N(C)C)ccc2n1CC1CC1 ; *c1c(F)c2cc(C(=O)N(C)C)ccc2n1CC1CC1 ; *c1ccc2cc(C(=O)N(C)CC)[nH]c2c1F]

In some embodiments, Ring W and its R^{w} substituents are

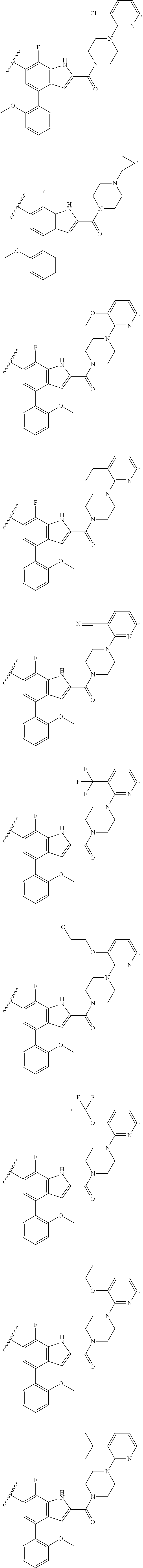

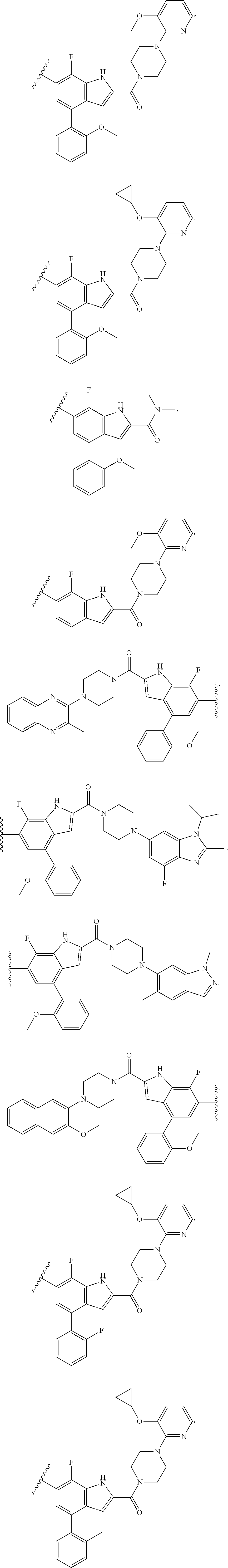

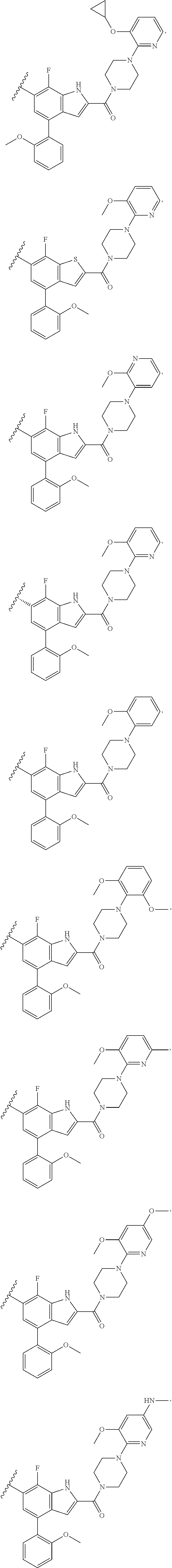

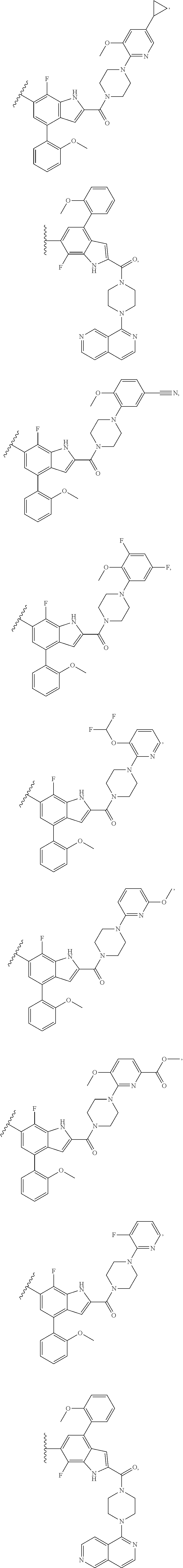

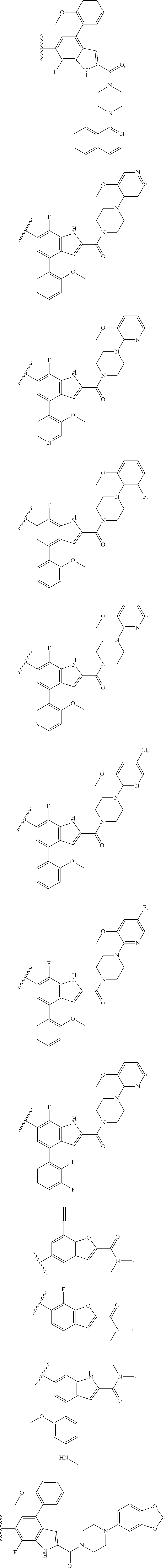

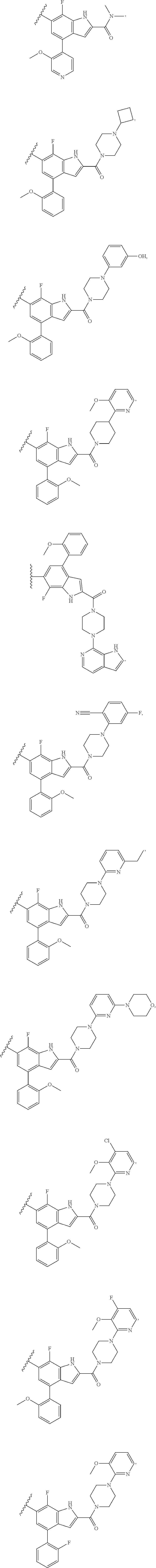

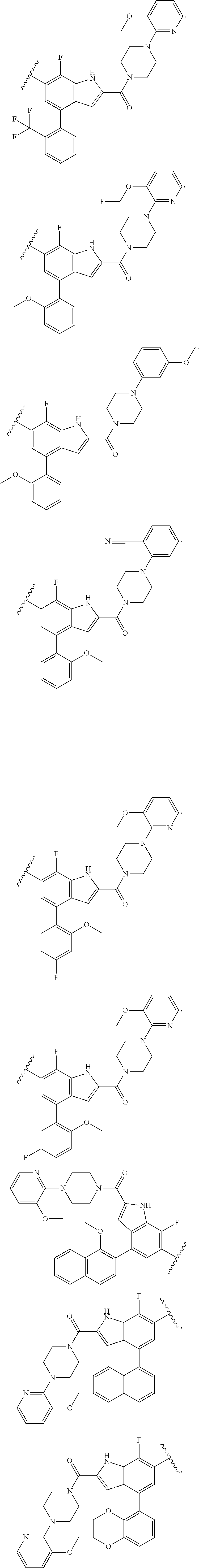

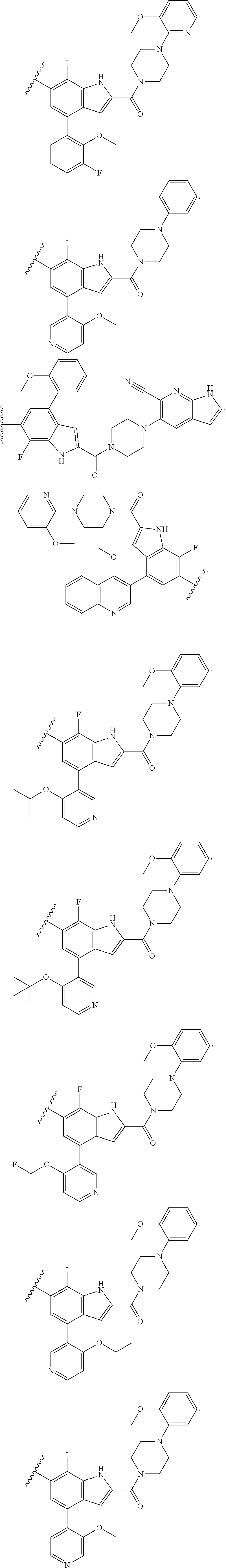

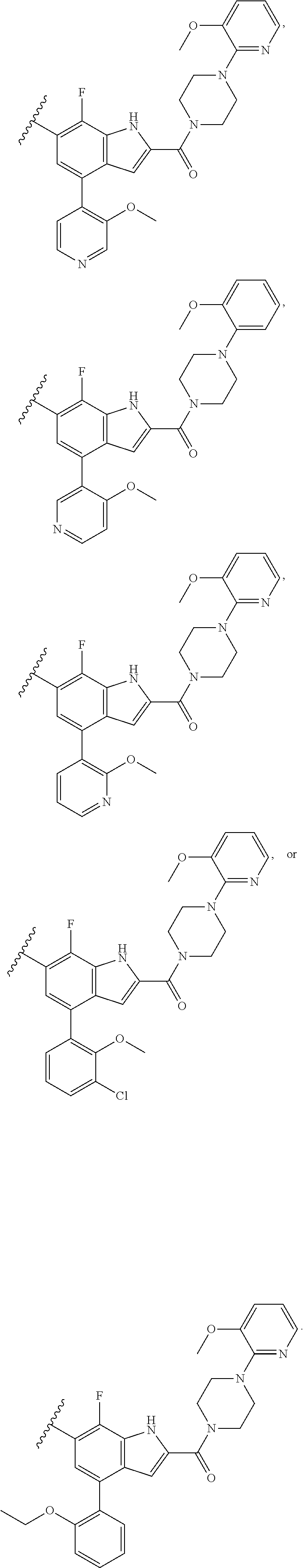 [SMILES: *c1cc(-c2ccncc2OC)c2cc(C(=O)N3CCN(c4ncccc4OC)CC3)[nH]c2c1F ; *c1cc(-c2cnccc2OC)c2cc(C(=O)N3CCN(c4ccccc4OC)CC3)[nH]c2c1F ; *c1cc(-c2cccnc2OC)c2cc(C(=O)N3CCN(c4ncccc4OC)CC3)[nH]c2c1F ; *c1cc(-c2cccc(Cl)c2OC)c2cc(C(=O)N3CCN(c4ncccc4OC)CC3)[nH]c2c1F ; *c1cc(-c2ccccc2OCC)c2cc(C(=O)N3CCN(c4ncccc4OC)CC3)[nH]c2c1F]

In some embodiments, Ring W is:

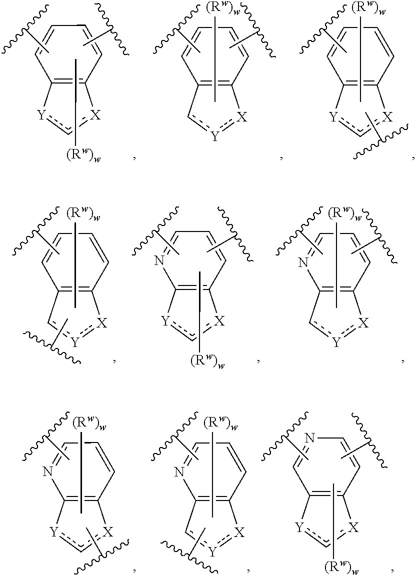

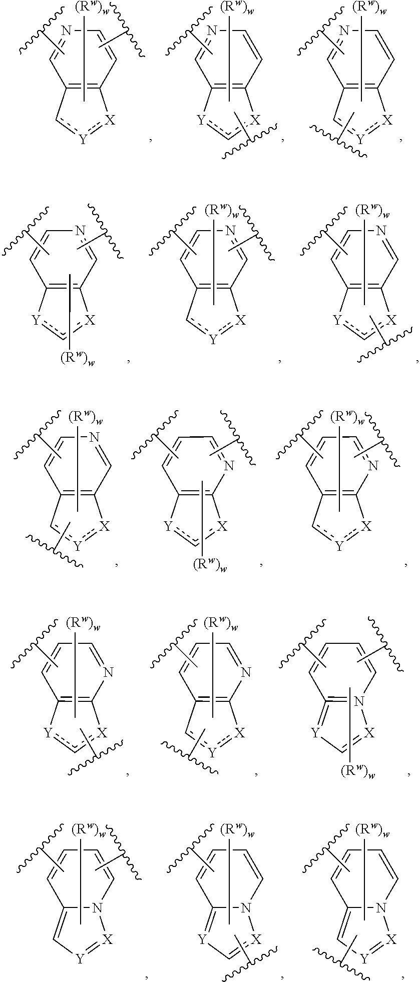

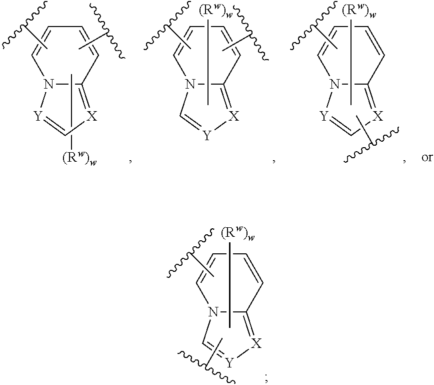

wherein R^{w} and w are as defined above and described herein; and
each of X and Y of Ring W is independently N, NH, N—W^{W}, —O—, —S—, C—H, C—W^{W}, C—H₂, CH(W^{W}), or C—(R^{W})₂.

In some embodiments, Ring W is:

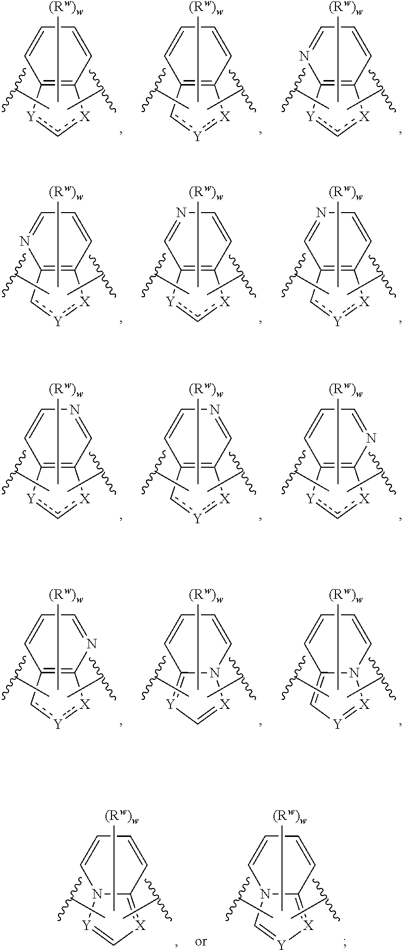

wherein each of R^{w}, w, X, and Y is as defined above and described here.

In some embodiments, Ring W is

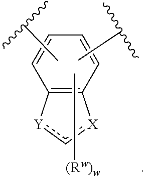

In some embodiments, Ring W is

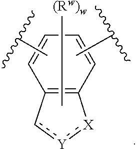

In some embodiments, Ring W is

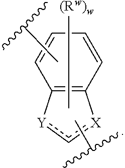

In some embodiments, Ring W is

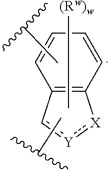

In some embodiments, Ring W is

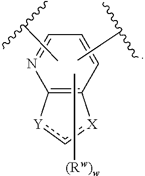

In some embodiments, Ring W is

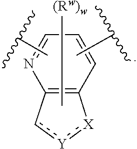

In some embodiments, Ring W is

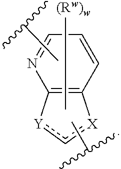

In some embodiments, Ring W is

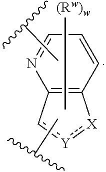

In some embodiments, Ring W is

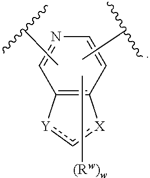

In some embodiments, Ring W is

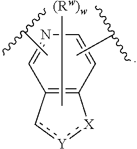

In some embodiments, Ring W is

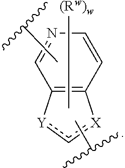

In some embodiments, Ring W is

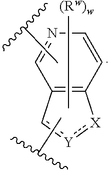

In some embodiments, Ring W is

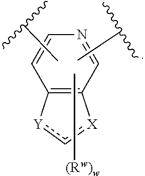

In some embodiments, Ring W is

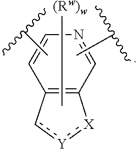

In some embodiments, Ring W is

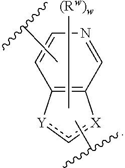

In some embodiments, Ring W is

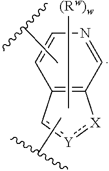

In some embodiments, Ring W is

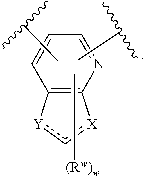

In some embodiments, Ring W is

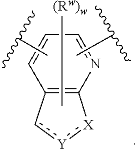

In some embodiments, Ring W is

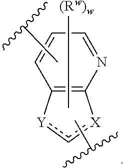

In some embodiments, Ring W is

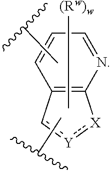

In some embodiments, Ring W is

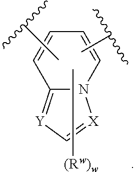

In some embodiments, Ring W is

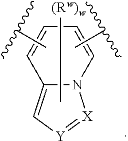

In some embodiments, Ring W is

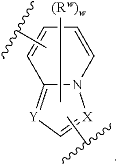

In some embodiments, Ring W is

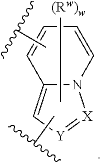

In some embodiments, Ring W is

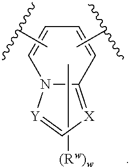

In some embodiments, Ring W is

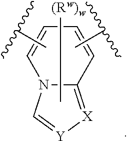

In some embodiments, Ring W is

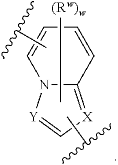

In some embodiments, Ring W is

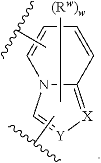

In some embodiments, each of X and Y of Ring W is independently N, NH, N—R^{W}, —O—, —S—, C—H, C—R^{W}, CH₂, CH(R^{W}), or C—(R^{w})₂.
In some embodiments, Ring W is

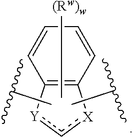

In some embodiments, Ring W is

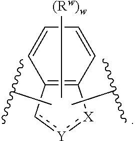

In some embodiments, Ring W is

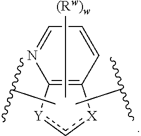

In some embodiments, Ring W is

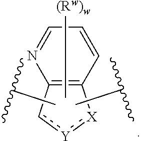

In some embodiments, Ring W is

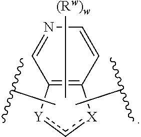

In some embodiments, Ring W is

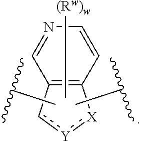

In some embodiments, Ring W is

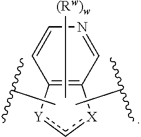

In some embodiments, Ring W is

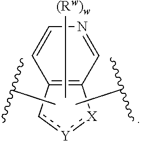

In some embodiments, Ring W is

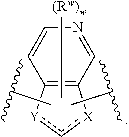

In some embodiments, Ring W is

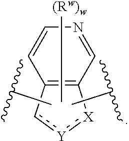

In some embodiments, Ring W is

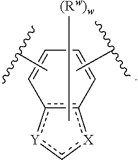

In some embodiments, Ring W is

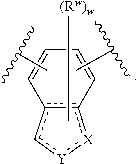

As defined above and described herein, each of X and Y of Ring W is independently N, NH, N—R^{W}, —O—, —S—, C—H, C—R^{W}, C—H₂, CH(R^{W}), or C—(R^{W})₂. In some embodiments, each of X and Y of Ring W is independently N, NH, N—R^{W}, —O—, —S—, C—H, C—R^{W}, C—H₂, CH(R^{W}), or C—(R^{W})₂, as allowed by valency.

In some embodiments, X is C—R^{w} or CH, and Y is N—R^{w}. In some embodiments, X is C—R^{w} or CH, and Y is S. In some embodiments, X is C—R^{w} or CH, and Y is O. In some embodiments, X is N—R^{w} or NH, and Y is C—R^{w} or CH. In some embodiments, X is S, and Y is C—R^{w} or CH. In some embodiments, X is O, and Y is C—R^{w} or CH.

In some embodiments, Ring W is

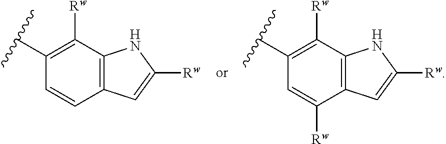

In some embodiments Ring W is

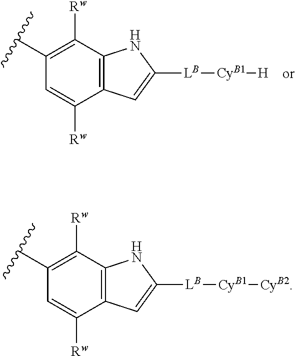

In some embodiments Ring W is

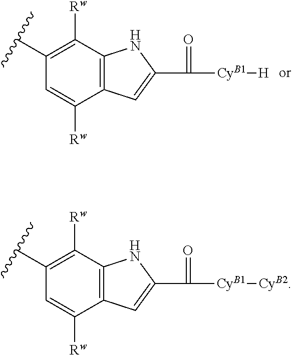

In some embodiments, Ring W is

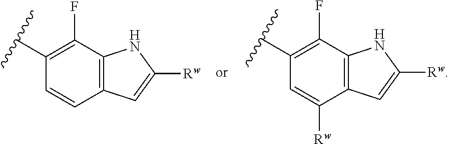

In some embodiments Ring W is

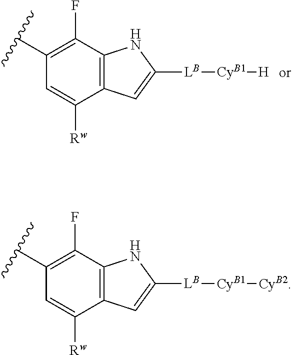

In some embodiments Ring W is

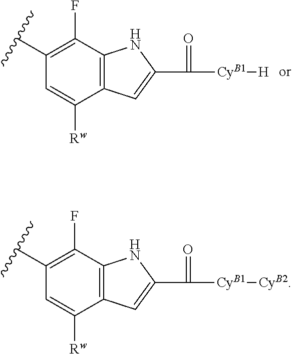

In some embodiments, Ring W is

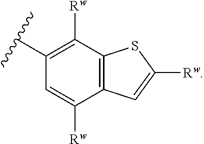

In some embodiments, Ring W is

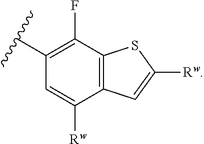

In some embodiments, Ring W is

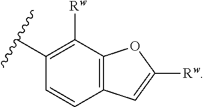

As described above and defined herein, Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen, and sulfur.

In some embodiments, Ring X is a ring selected from phenylenyl, 4-7 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen, and sulfur.

In some embodiments, Ring X is a ring selected from a 3-8 membered saturated or partially unsaturated carbocyclylenyl or heterocyclylenyl with 1-3 heteroatoms independently selected from nitrogen, oxygen, and sulfur. In some embodiments, Ring X is a ring selected from a 3-8 membered saturated or partially unsaturated carbocyclylenyl. In some embodiments, Ring X is a ring selected from a 3-8 membered saturated or partially unsaturated heterocyclylenyl with 1-3 heteroatoms independently selected from nitrogen, oxygen, and sulfur.

In some embodiments, Ring X is phenylenyl. In some embodiments, Ring X is a 4-7 membered saturated or partially unsaturated carbocyclylenyl. In some embodiments, Ring X is a 4-7 membered saturated or partially unsaturated heterocyclylenyl with 1-3 heteroatoms independently selected from nitrogen, oxygen, and sulfur. In some embodiments, Ring X is a 5-6 membered heteroarylenyl with 1-4 heteroatoms independently selected from nitrogen, oxygen, and sulfur.

In some embodiments, Ring X is a 6-membered saturated or partially unsaturated heterocyclylenyl with 1-3 heteroatoms independently selected from nitrogen, oxygen, and sulfur. In some embodiments, Ring X is a 6-membered saturated or partially unsaturated heterocyclylenyl having 1 nitrogen atom.

In some embodiments, Ring X is a ring selected from a 5-6 membered saturated or partially unsaturated heterocyclylenyl with 1-3 heteroatoms independently selected from nitrogen, oxygen, and sulfur. In some embodiments, Ring X is a ring selected from a 6-7 membered saturated or partially unsaturated heterocyclylenyl with 1-3 heteroatoms independently selected from nitrogen, oxygen, and sulfur. In embodiments, Ring X is a 7-membered saturated or partially unsaturated heterocyclylenyl with 1-3 heteroatoms independently selected from nitrogen, oxygen, and sulfur. In some embodiments, Ring X is a 7-membered saturated or partially unsaturated heterocyclylenyl having 1 nitrogen atom.

In some embodiments, Ring W is

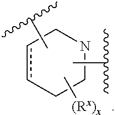

In some embodiments, Ring W is

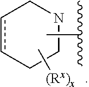

In some embodiments, Ring W is

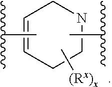

In some embodiments, Ring W is

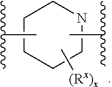

In some embodiments, Ring W is

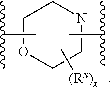

In some embodiments, Ring W is

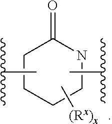

In some embodiments, Ring W is

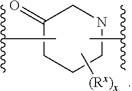

In some embodiments, Ring W is

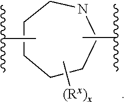

In some embodiments, Ring W is

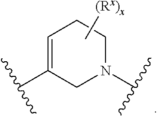

In some embodiments, Ring W is

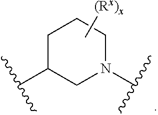

In some embodiments, Ring W is

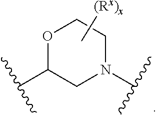

In some embodiments, Ring W is

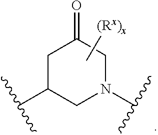

In some embodiments, Ring W is

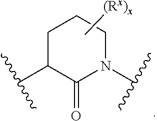

In some embodiments, Ring W is

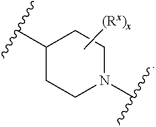

In some embodiments, Ring W is

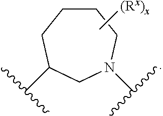

In some embodiments, Ring W is

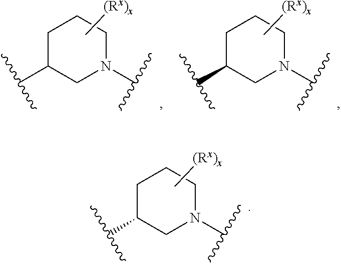

In some embodiments, Ring W is

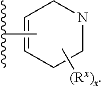

In some embodiments, Ring W is

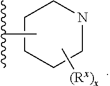

In some embodiments, Ring W is

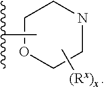

In some embodiments, Ring W is

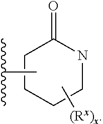

In some embodiments, Ring W is

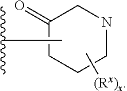

In some embodiments, Ring W is

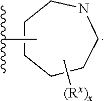

In some embodiments, Ring W is

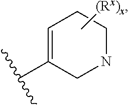

In some embodiments, Ring W is

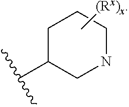

In some embodiments, Ring W is

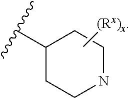

In some embodiments, Ring W is

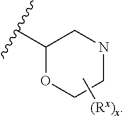

In some embodiments, Ring W is

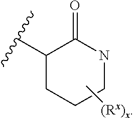

In some embodiments, Ring W is

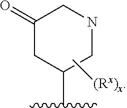

In some embodiments, Ring W is

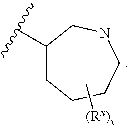

In some embodiment, Ring X is as depicted in the compounds of Table 1A, below.

In some embodiments, Ring X and its R^{X} substituents are

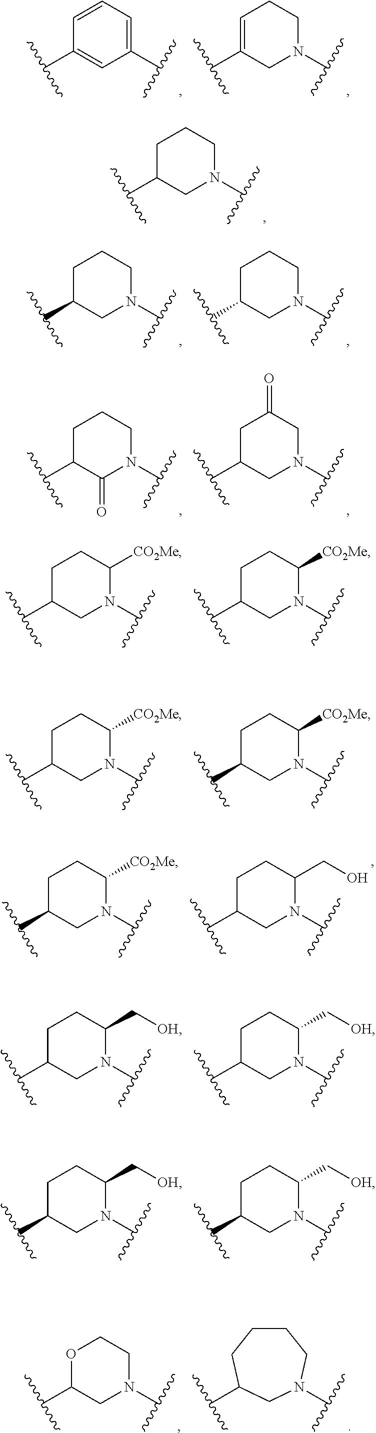

In some embodiments, Ring X and its R^{X} substituents are

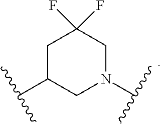 [SMILES: *C1CN(*)CC(F)(F)C1]

In some embodiments, Ring X and its R^{X} substituents are

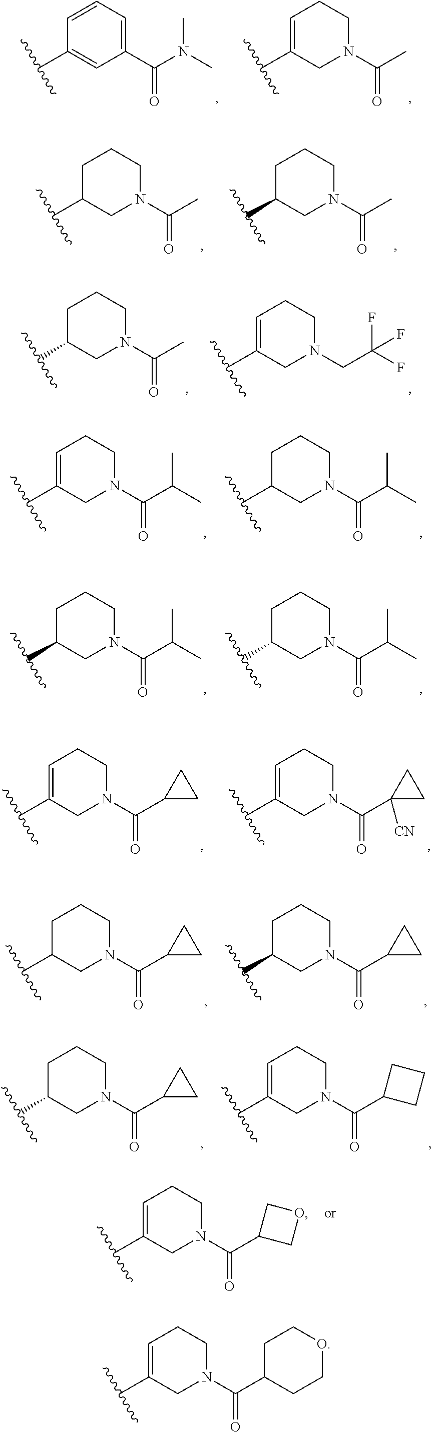

As described above and defined herein, G is hydrogen or

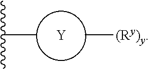

In some embodiments, G is hydrogen. In some embodiments, G is

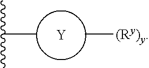

In some embodiment, G is as depicted in the compounds of Table 1A, below.

As described above and defined herein, Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen, and sulfur.

In some embodiments, Ring Y is phenyl. In some embodiments, Ring Y is naphthyl. In some embodiments, Ring Y is a 3-11 membered saturated or partially unsaturated monocyclic, bicyclic, bridged bicyclic, or spirocyclic carbocyclyl. In some embodiments, Ring Y is a 5-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring Y is a 5-10 membered monocyclic or bicyclic heteroaryl with 1-4 heteroatoms independently selected from nitrogen, oxygen, and sulfur.

In some embodiments, Ring Y is a 3-8 membered saturated or partially unsaturated monocyclic carbocyclyl. In some embodiments, Ring Y is a 5-6 membered saturated or partially unsaturated monocyclic carbocyclyl. In some embodiments, Ring Y is a 6-7 membered saturated or partially unsaturated monocyclic carbocyclyl. In some embodiments, Ring Y is a 3 membered saturated or partially unsaturated monocyclic carbocyclyl. In some embodiments, Ring Y is a 4 membered saturated or partially unsaturated monocyclic carbocyclyl. In some embodiments, Ring Y is a 5 membered saturated or partially unsaturated monocyclic carbocyclyl. In some embodiments, Ring Y is a 6 membered saturated or partially unsaturated monocyclic carbocyclyl. In some embodiments, Ring Y is a 7 membered saturated or partially unsaturated monocyclic carbocyclyl. In some embodiments, Ring Y is cyclopropyl, cyclobutyl, cyclopentyl, cyclohexyl, or cycloheptyl.

In some embodiments, Ring Y is a 3-6 membered saturated or partially unsaturated monocyclic carbocyclyl. In some embodiments, Ring Y is cyclopropyl. In some embodiments, Ring Y is cyclobutyl. In some embodiments, Ring Y is cyclopentyl. In some embodiments, Ring Y is hexyl.

In some embodiments, Ring Y is a 5-6 membered monocyclic heteroaryl with 1-4 heteroatoms independently selected from nitrogen, oxygen, and sulfur. In some embodiments, Ring Y is a 9-membered bicyclic heteroaryl with 1-4 heteroatoms independently selected from nitrogen, oxygen, and sulfur. In some embodiments, Ring Y is a 5-membered monocyclic heteroaryl with 1-4 heteroatoms independently selected from nitrogen, oxygen, and sulfur.

In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 4 heteroatoms independently selected from nitrogen, oxygen, and sulfur. In some embodiments, Ring Y is a 5-membered monocyclic heteroaryl ring with 2-3 nitrogen heteroatoms.

In some embodiments, Ring Y is a 6-membered monocyclic heteroaryl with 1-4 heteroatoms independently selected from nitrogen, oxygen, and sulfur. In some embodiments, Ring Y is a 6-membered monocyclic heteroaryl with 1-4 nitrogen heteroatoms. In some embodiments, Ring Y is pyridinyl, pyrimidinyl, pyridazinyl, or triazinyl.

In some embodiments, Ring Y is an 8-11 membered saturated or partially unsaturated bicyclic, bridged bicyclic, or spirocyclic heterocyclyl with 1-3 heteroatoms independently selected from nitrogen, oxygen, and sulfur. In some embodiments, Ring Y is a 8-11 membered saturated or partially unsaturated bicyclic heterocyclyl with 1-3 heteroatoms independently selected from nitrogen, oxygen, and sulfur. In some embodiments, Ring Y is a 9-10 membered saturated or partially unsaturated bicyclic heterocyclyl with 1-3 heteroatoms independently selected form nitrogen, oxygen, and sulfur. In some embodiments, Ring Y is a 9 membered saturated or partially unsaturated bicyclic heterocyclyl with 1-3 heteroatoms independently selected from nitrogen, oxygen, and sulfur. In some embodiments, Ring Y is a 10 membered saturated or partially unsaturated bicyclic heterocyclyl with 1-3 heteroatoms independently selected from nitrogen, oxygen, and sulfur. In some embodiments, Ring Y is tetrahydroisoquinolinyl or tetrahydroquinolinyl.

In some embodiments, Ring Y is a 9-10 membered bicyclic heteroaryl with 1-4 heteroatoms independently selected from nitrogen, oxygen, and sulfur. In some embodiments, Ring Y is a 9 membered bicyclic heteroaryl with 1-4 heteroatoms independently selected from nitrogen, oxygen, and sulfur. In some embodiments, Ring Y is a 9 membered bicyclic heteroaryl with 1-4 nitrogen heteroatoms.

In some embodiments, Ring Y is

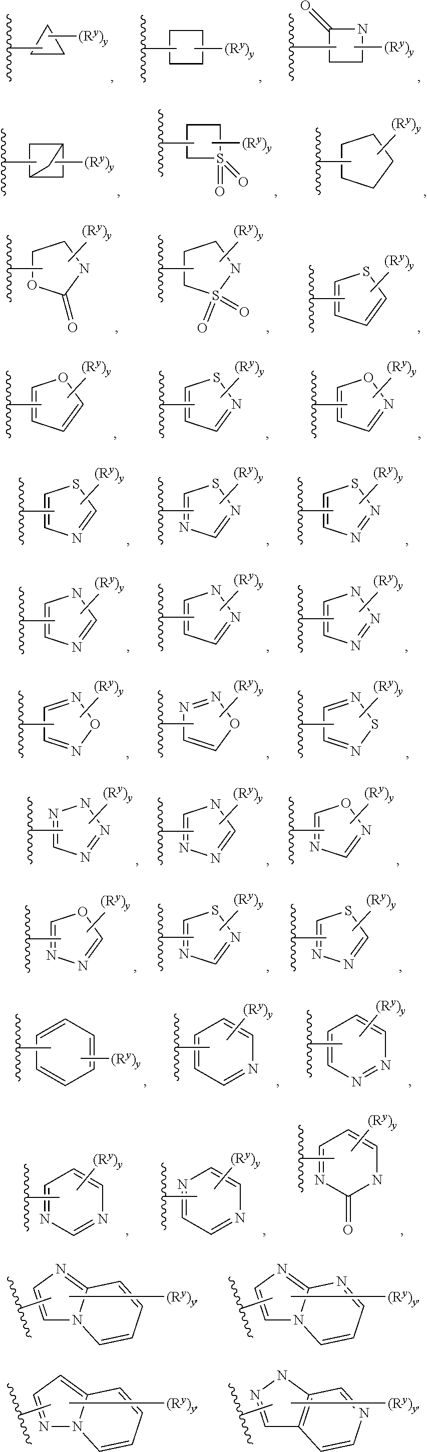

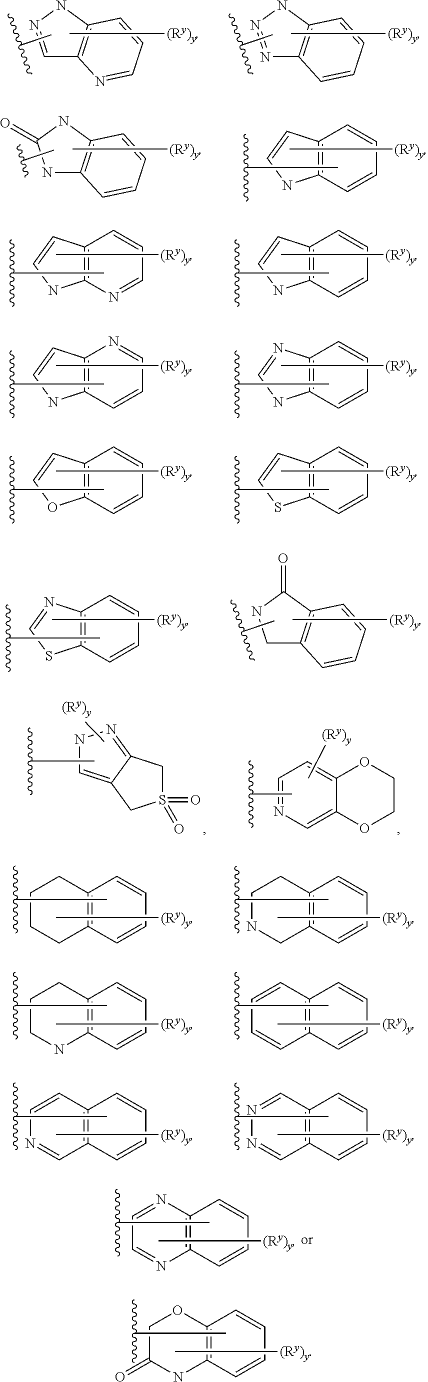

In some embodiments, Ring Y is

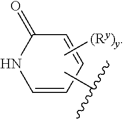

In some embodiments, Ring Y is

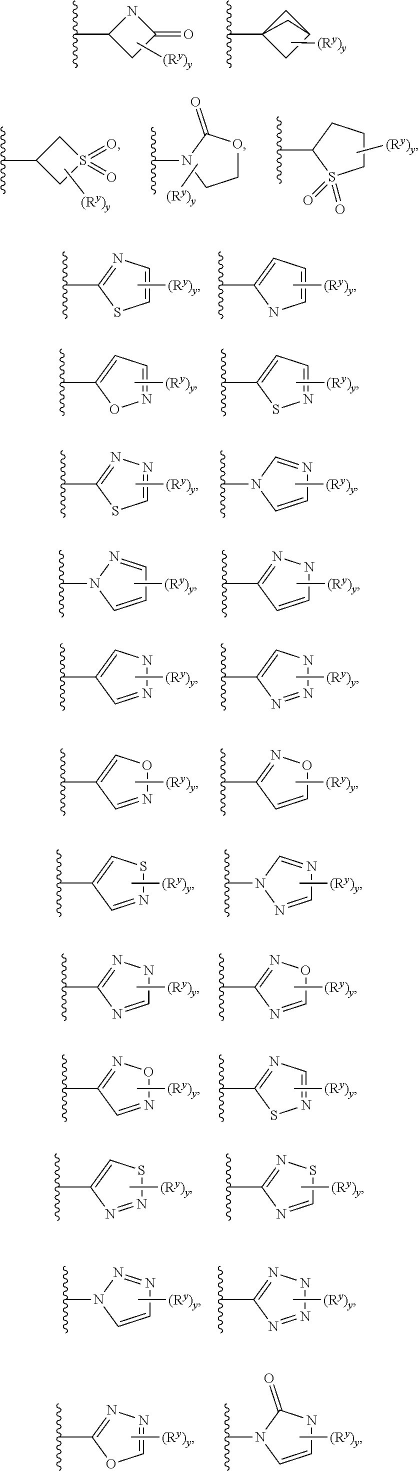

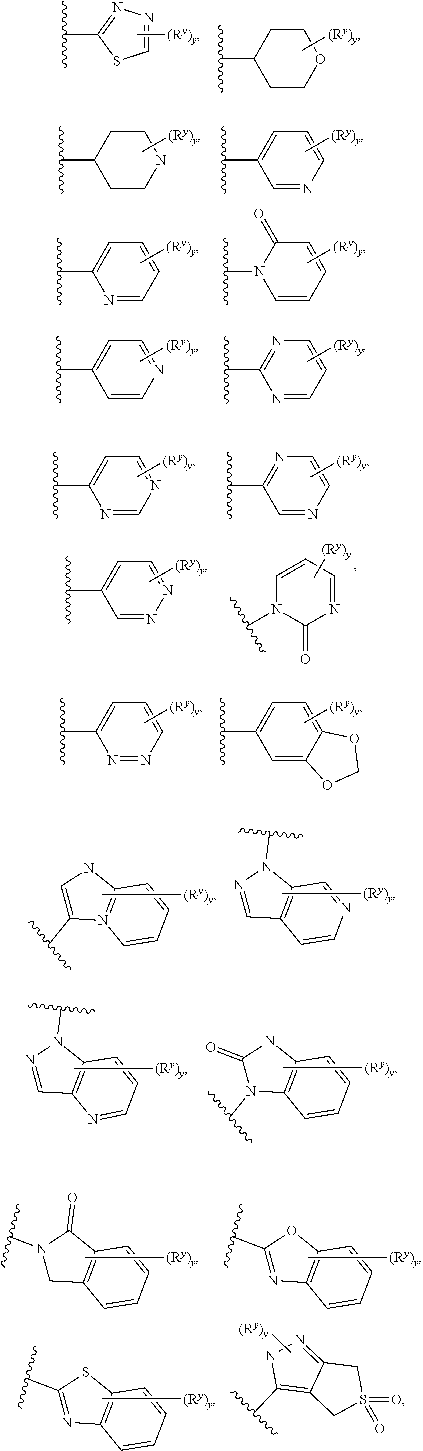

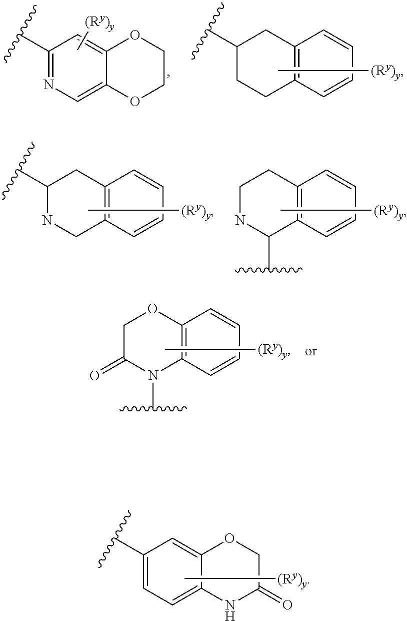

In some embodiment, Ring Y is

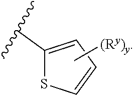

In some embodiment, Ring Y is as depicted in the compounds of Table 1A, below.

In some embodiments, Ring Y and its R^{y} substituents are

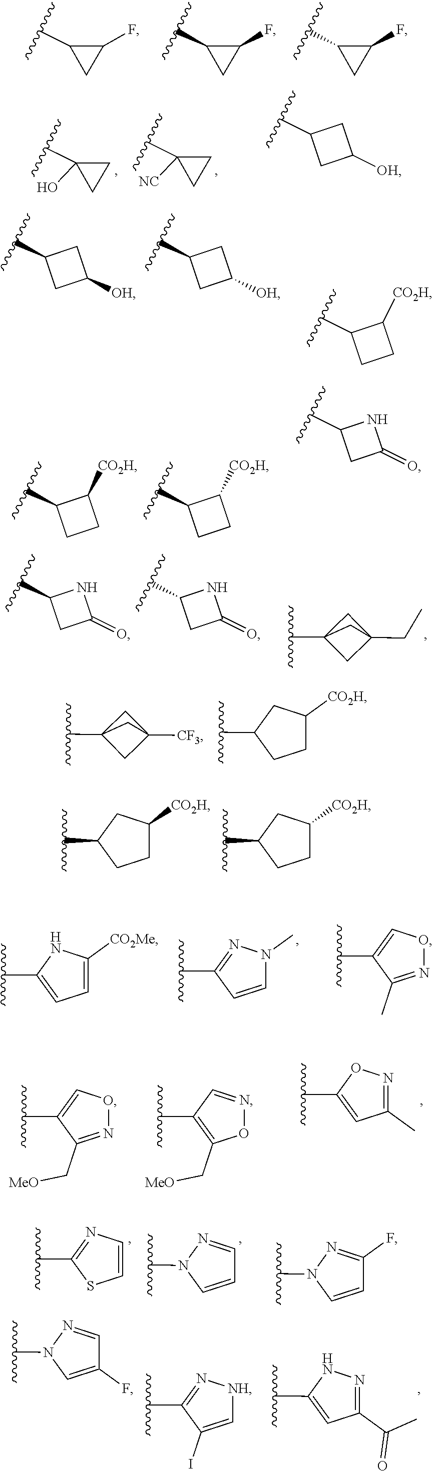

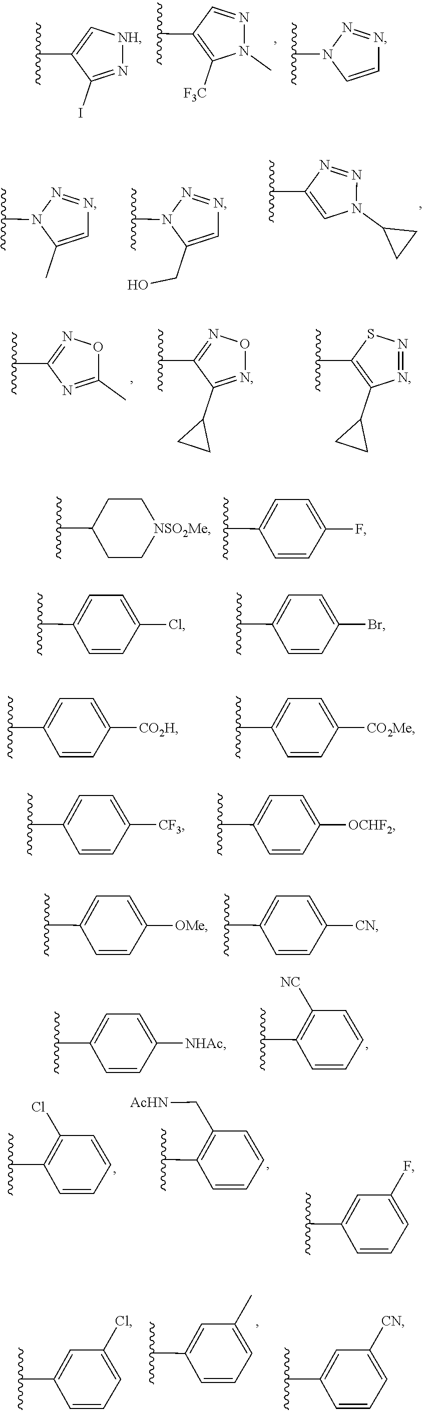

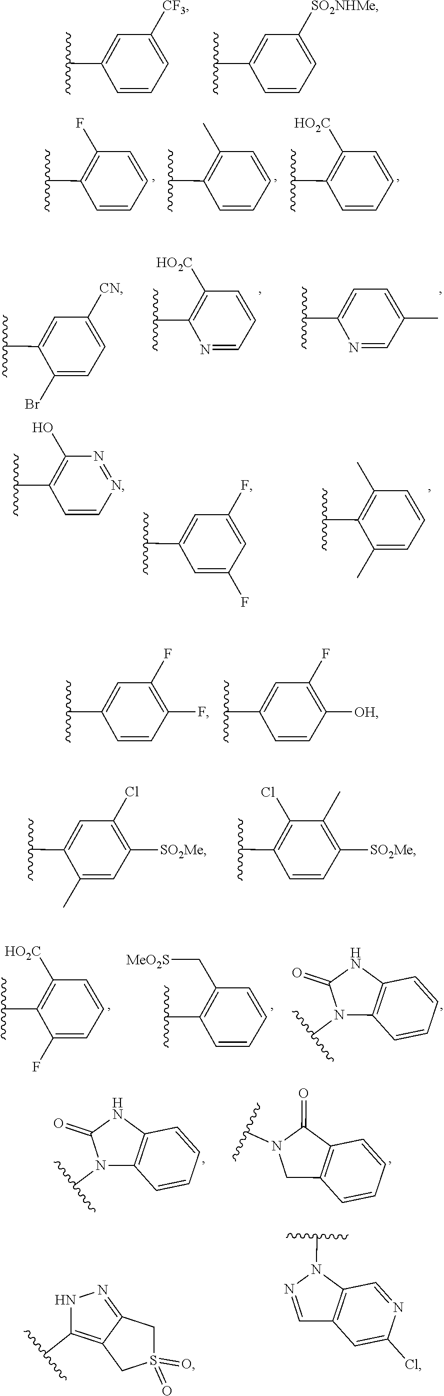

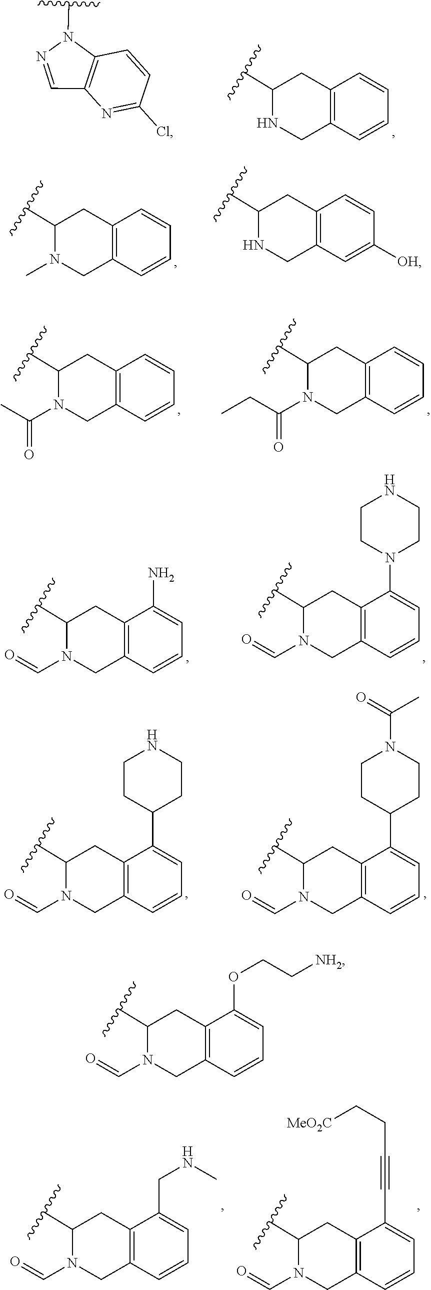

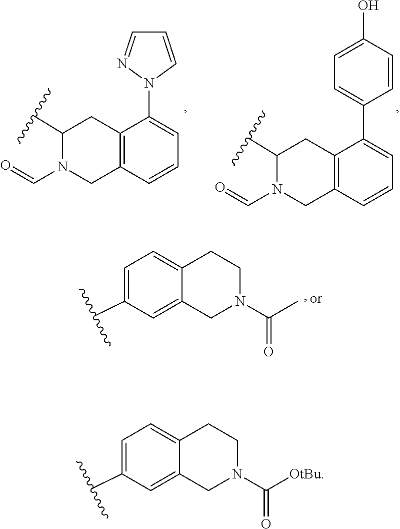 [SMILES: *C1Cc2c(cccc2-n2cccn2)CN1C=O ; *C1Cc2c(cccc2-c2ccc(O)cc2)CN1C=O ; *c1ccc2c(c1)CN(C(C)=O)CC2 ; *c1ccc2c(c1)CN(C(=O)OC(C)(C)C)CC2]

In some embodiments, Ring Y and its R^{y} substituents are

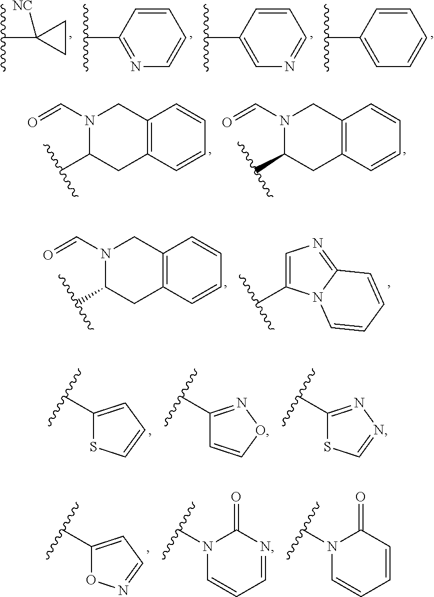

In some embodiments, Ring Y is

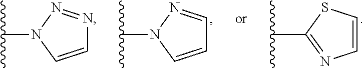

As described above and defined herein, R^{x}, and R^{y} are independently selected from hydrogen, R^{A}, halogen, —CN, —NO₂, —OR—SR—NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from hydrogen. R^{A}, halogen, —CN, —NO₂, —OR, —SR, —NR₂, —SiR₃, —S(O)R, —S(O)₂R, —S(O)(NR)R, —S(O)₂NR, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{w} is hydrogen. In some embodiments, R^{w} is R^{A}. In some embodiments, R^{w} is halogen. In some embodiments, R^{w} is —CN. In some embodiments, R^{w} is —NO₂. In some embodiments, R^{w} is —OR. In some embodiments, R^{w} is —SR. In some embodiments, R^{w} is —NR₂. In some embodiments, R^{w} is —SiR₃. In some embodiments, R^{w} is —S(O)₂R. In some embodiments, R^{w} is —S(O)₂NR₂. In some embodiments, R^{w} is —S(O)(NR)R. In some embodiments, R^{w} is —S(O)R. In some embodiments, R^{w} is —C(O)R. In some embodiments, R^{w} is —C(O)OR. In some embodiments, R^{w} is —C(O)NR₂. In some embodiments, R^{w} is —C(O)NROR. In some embodiments, R^{w} is —OC(O)R. In some embodiments, R^{w} is —OC(O)NR₂. In some embodiments, R^{w} is —P(O)R₂. In some embodiments, R^{w} is —P(O)(OR)₂. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —NRC(O)OR. In some embodiments, R^{w} is —NRC(O)R. In some embodiments, R^{w} is —NRC(O)N(R)₂. In some embodiments, R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is fluoro, chloro, or bromo.

In some embodiments, R^{w} is —C(O)NHR. In some embodiments, R^{w} is —C(O)NHR, wherein R of R^{w} is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{w} is optionally substituted C₁₋₆ aliphatic. In some embodiments, R^{w} is C₁₋₆ aliphatic, optionally substituted with —C(O)N(R^{o})₂. In some embodiments, R^{w} is C₁₋₆ aliphatic, optionally substituted with —NR^{o}(O)N(R^{o})₂. In some embodiments, R^{w} is

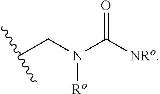

In some such embodiments, R^{o} is hydrogen or C₁₋₆ aliphatic. In some embodiments, R^{w} is C₁₋₆ aliphatic optionally substituted with —OR^{o}, wherein R^{o} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, R^{w} is C₁₋₆ aliphatic optionally substituted with halogen (e.g., fluoro). In some embodiments, R^{w} is

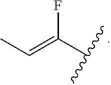 [SMILES: */C(F)=C/C ; "."]

In some embodiments, R^{w} is C₁₋₆ aliphatic optionally substituted with halogen (e.g., fluoro) or —NR^{o}₂. In some embodiments, R^{w} is C₁₋₆ aliphatic optionally substituted with halogen (e.g., fluoro) and —NR^{o}₂. In some embodiments, R^{w} is C₆ aliphatic optionally substituted with halogen (e.g., fluoro) or —NR^{o}₂, wherein each R^{o} is independently hydrogen or C₁₋₆ aliphatic. In some embodiments, R^{w} is C₁₋₆ aliphatic optionally substituted with halogen (e.g., fluoro) and —NR^{o}₂, wherein each R^{o} is independently hydrogen or C₁₋₆ aliphatic. In some embodiments, R^{w} is R^{w} is

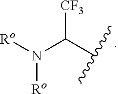

In some embodiments, R^{w} is C₁₋₆ aliphatic optionally substituted with —(CH₂)₀₋₄R^{o}, wherein R^{o} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —(CH₂)₀₋₄R^{o}, wherein R^{o} is a C₁₋₆ aliphatic. In some embodiments, R^{w} is C₁₋₆ aliphatic optionally substituted with —R^{o}, wherein R^{o} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —R^{o}, wherein R^{o} is a C₁₋₆ aliphatic.

In some embodiments, R^{w} is C₁₋₆ aliphatic optionally substituted with —(CH₂)₀₋₄R^{o}, wherein R^{o} is cyclopropyl, cyclobutyl, cyclopentyl, or cyclohexyl. In some embodiments, R^{w} is C₁₋₆ aliphatic optionally substituted with —R^{o}, wherein R^{o} is a cyclopropyl, cyclobutyl, cyclopentyl, or cyclohexyl.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 5-6 membered saturated or partially unsaturated carbocyclic or heterocyclic ring having 1-2 heteroatoms independently selected from nitrogen, oxygen, and sulfur.

In some embodiments R^{w} is an optionally substituted phenyl.

In some embodiments R^{w} is an optionally substituted 3-7 membered saturated or partially unsaturated carbocyclic. In some embodiments R^{w} is an optionally substituted 3-7 membered saturated or partially unsaturated carbocyclic. In some embodiments R^{w} is an optionally substituted cyclopropyl. In some embodiments R^{w} is an optionally substituted cyclobutyl. In some embodiments R^{w} is an optionally substituted cyclopentyl. In some embodiments R^{w} is an optionally substituted cyclohexyl. In some embodiments R^{w} is an optionally substituted cyclopropenyl. In some embodiments R^{w} is an optionally substituted cyclobutenyl. In some embodiments R^{w} is an optionally substituted cyclopentenyl. In some embodiments R^{w} is an optionally substituted cyclohexenyl.

In some embodiments R^{w} is an optionally substituted 3-7 membered saturated or partially unsaturated heterocyclic ring having 1-2 heteroatoms independently selected from nitrogen, oxygen, and sulfur. In some embodiments R^{w} is an optionally substituted 5-6 membered saturated or partially unsaturated heterocyclic ring having 1-2 heteroatoms independently selected from nitrogen, oxygen, and sulfur.

In some embodiments R^{w} is an optionally substituted 4 membered saturated or partially unsaturated heterocyclic ring having 1-2 heteroatoms independently selected from nitrogen, oxygen, and sulfur. In some embodiments R^{w} is an optionally substituted azetidinyl, oxetanyl, or thietanyl.

In some embodiments R^{w} is an optionally substituted 5 membered saturated or partially unsaturated heterocyclic ring having 1-2 heteroatoms independently selected from nitrogen, oxygen, and sulfur. In some embodiments, R^{w} is an optionally substituted pyrrolidinyl, pyrrolinyl, pyrazolidinyl, pyrazolinyl, imidazolidinyl, or imidazolinyl. In some embodiments, R^{w} is an optionally substituted dihydropyridinyl, pyrrolidinyl, dihydrofuranyl, tetrahydrofuranyl, dihydrothiophenyl, or tetrahydrothiophenyl.

In some embodiments R^{w} is an optionally substituted 6 membered saturated or partially unsaturated heterocyclic ring having 1-2 heteroatoms independently selected from nitrogen, oxygen, and sulfur. In some embodiments R^{w} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithianyl, 1,4-dithianyl, morpholinyl, or thiomorpholinyl. In some embodiments, R^{w} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w} is an optionally substituted

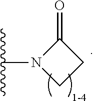

In some embodiments, R^{w} is an optionally substituted

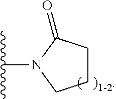

In some embodiments, R^{w} is an optionally substituted

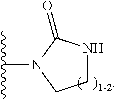

In some embodiments, R^{w} is

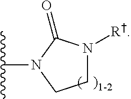

In some embodiments, R^{w} is optionally substituted

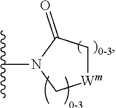

where W^{m} is O, S, C(O), or NR^{o}, wherein R^{o} is as described above and defined herein. In some embodiments, R^{w} is

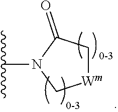

In some embodiments, R^{w} is optionally substituted

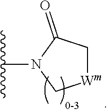

In some embodiments, R^{w} is

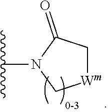

In some embodiments, R^{w} is

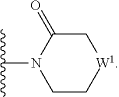

In some embodiments, R^{w} is

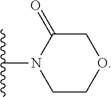 [SMILES: *N1CCOCC1=O]

In some embodiments, W^{m} is O. In some embodiments, W^{m} is S. In some embodiments, W^{m} is C(O). In some embodiments, W^{m} is NR^{o}. In some embodiments, W^{m} is NR^{o}, wherein R^{o} is hydrogen or C₁₋₆ aliphatic.

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{o})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{o})₂. †

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R is optionally substituted pyridinyl, pyrimidinyl, pyridazinyl, or triazinyl. In some embodiments, R^{w} is an optionally substituted pyridinonyl, pyrazinonyl, or pyrimidinonyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In some embodiments, R^{w} is —S(O)₂NH₂. In some embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)H. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{W} is —C(S)R, —C(NR)R, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R—S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R—S(O)₂NR₂, —S(O)R, or —NRS(O)₂R. In some embodiments, R^{w} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R—S(O)₂NR₂, —S(O)R, —C(S)R, —C(NR)R, —C(O)R, —C(O)OR, —C(O)NR₂, or —C(O)NROR. In some embodiments, R^{w} is —C(S)R, —C(NR)R, —C(O)R, —C(O)OR, or —C(O)NR₂.

In some embodiments, R^{w} is —C(O)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w} is —C(S)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OH. In some embodiments, R^{w} is —C(O)OR, wherein R is of R^{w} is optionally substituted C₁₋₆ aliphatic. In some embodiments, R^{w} is —C(O)OR, wherein R is of R is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{W} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(NR)R. In some embodiments, R^{w} is —C(NH)R. In some embodiments, R^{w} is C(NR)R, wherein each R is of R^{w} is independently hydrogen or an optionally substituted C₁₋₆ aliphatic. In some embodiments, R^{w} is C(NR)R, wherein each R is of R^{w} is independently hydrogen or an optionally substituted phenyl. In some embodiments, R^{w} is C(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is C(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R^{w} is —C(O)NR₂, wherein the two R groups of R^{w} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)H. In some embodiments, R^{w} is —OC(O)R, wherein R is of R^{w} is optionally substituted C₁₋₆ aliphatic. In some embodiments, R is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{W} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{w} is —NRC(O)OR, wherein each R is of R is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)₂, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)H. In some embodiments, R^{w} is —S(O)R, wherein R is of R^{w} is optionally substituted C₁₋₆ aliphatic. In some embodiments, R^{w} is —S(O)R, wherein R is of R^{w} is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂H. In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R^{w} is independently hydrogen or optionally substituted phenyl. In some embodiments, R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of RV is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R is independently hydrogen or an optionally substituted C₁₋₆ aliphatic. In some embodiments, R^{w} is —S(O)₂NR₂, wherein each R is of WV is independently hydrogen or an optionally substituted phenyl. In some embodiments, R is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

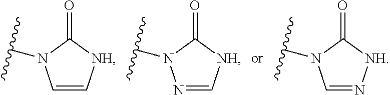

In some embodiments, R^{w} is:

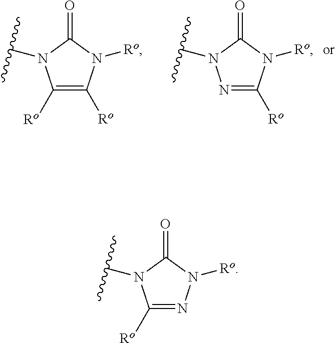

In some such embodiments, R^{o} is hydrogen or C₁₋₆ aliphatic. In some embodiments, R^{w} is:

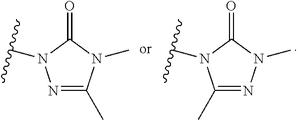

In some embodiments, R is an optionally substituted:

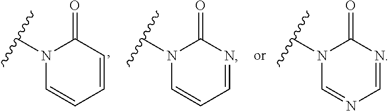

In some embodiments, R^{w} is:

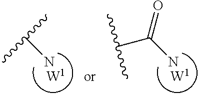

wherein Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.

As defined above and described herein, Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur. In some embodiments, Ring W¹ is an optionally substituted 3-7 membered saturated or partially unsaturated heterocyclic ring having 1 additional heteroatom selected from nitrogen, oxygen, and sulfur. In some embodiments, Ring W¹ is an optionally substituted 5-6 membered saturated or partially unsaturated heterocyclic ring having 1 additional heteroatom selected from nitrogen, oxygen, and sulfur. In some embodiments, Ring W¹ is an optionally substituted 5-6 membered monocyclic heteroaryl ring having 1-3 additional heteroatoms independently selected from nitrogen, oxygen, and sulfur. In some embodiments, Ring W¹ is an optionally substituted 5 membered monocyclic heteroaryl ring having 1-3 additional heteroatoms independently selected from nitrogen, oxygen, and sulfur. In some embodiments, Ring W¹ is an optionally substituted 6 membered monocyclic heteroaryl ring having 1-3 additional heteroatoms independently selected from nitrogen, oxygen, and sulfur.

In some embodiments, R^{w} is:

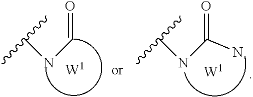

In some embodiments, R^{w} is:

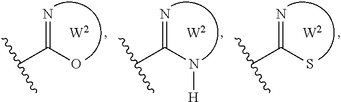

wherein Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

As defined above and described herein, Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur. In some embodiments, Ring W² is an optionally substituted 3-7 membered partially unsaturated heterocyclic ring. In some embodiments, Ring W² is an optionally substituted 5-6 membered partially unsaturated heterocyclic ring. In some embodiments, Ring W² is an optionally substituted 5-6 membered monocyclic heteroaryl ring having 1-2 additional heteroatoms independently selected from nitrogen, oxygen, and sulfur. In some embodiments, Ring W² is a 5 membered monocyclic heteroaryl ring having 1-2 additional heteroatoms independently selected from nitrogen, oxygen, and sulfur. In some embodiments, Ring W² is an optionally substituted oxazolyl, imidazolyl, thiazolyl, 1,34-thiadiazolyl, 2-imidazolinyl, 1,24-triazolyl, 1,24-oxadiazolyl, or 1,3,4-oxadiazolyl.

In some embodiments, R^{w} is an optionally substituted ring selected from:

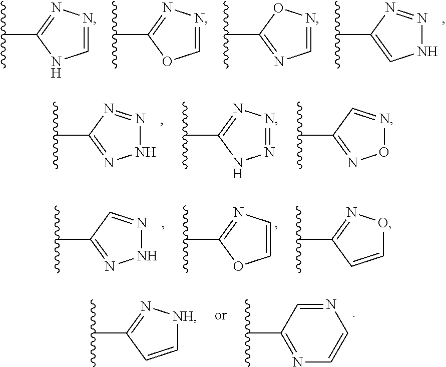

In some embodiments, R^{w} is:

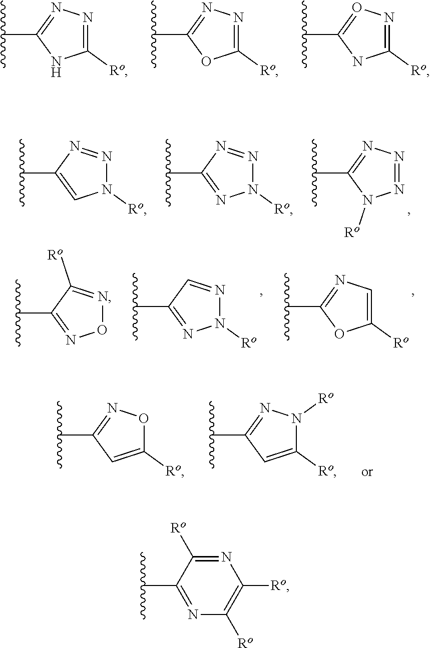

wherein each R^{o} is a defined above and described herein (e.g., hydrogen or C₁₋₆ aliphatic.

In some embodiments, R^{w} is:

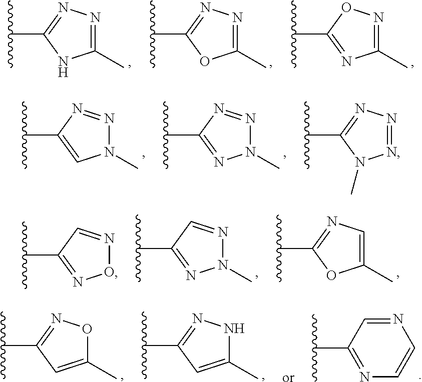

In some embodiments R^{w} is an optionally substituted 7-11 membered bicyclic heterocyclic ring having 1-4 heteroatoms independently selected from nitrogen, oxygen, and sulfur. In some embodiments R^{w} is an optionally substituted 9-10 membered bicyclic heterocyclic ring having 1-4 heteroatoms independently selected from nitrogen, oxygen, and sulfur. In some embodiments R^{w} is an optionally substituted benzo[d][1,3]dioxolyl. In some embodiments R^{w} is an optionally substituted an 11 membered bicyclic heterocyclic ring having 1-4 heteroatoms independently selected from nitrogen, oxygen, and sulfur.

In some embodiments R^{w} is an optionally substituted 8-11 membered bicyclic aryl or heteroaryl having 1-4 heteroatoms independently selected from nitrogen, oxygen and sulfur. In some embodiments, R^{w} is an optionally substituted naphthalenyl. In some embodiments, R^{w} is an optionally substituted dihydrobenzodioxepinyl. In some embodiments, R^{w} is an optionally substituted indenyl or dihydroindenyl.

In some embodiments, R^{w} is an optionally substituted 9-10 bicyclic heteroaryl having 1-4 heteroatoms independently selected from nitrogen, oxygen and sulfur.

In some embodiments R^{w} is an optionally substituted 9 bicyclic heteroaryl having 1-4 heteroatoms independently selected from nitrogen, oxygen and sulfur. In some embodiments R^{w} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments R^{w} is an optionally substituted 10 bicyclic heteroaryl having 1-4 heteroatoms independently selected from nitrogen, oxygen and sulfur. In some embodiments R^{w} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w} is R^{B}.

In some embodiments, R^{w} is fluoro, chloro, —CN, methyl, —CF₃, —CHF₂, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

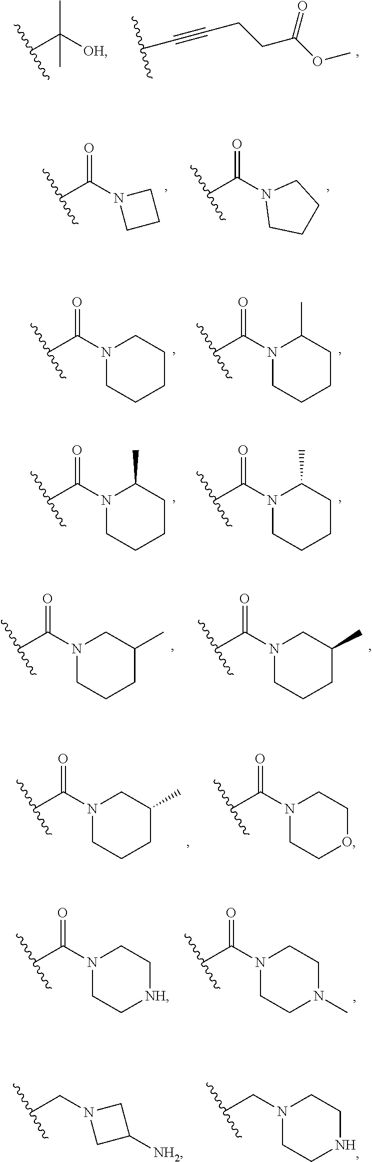

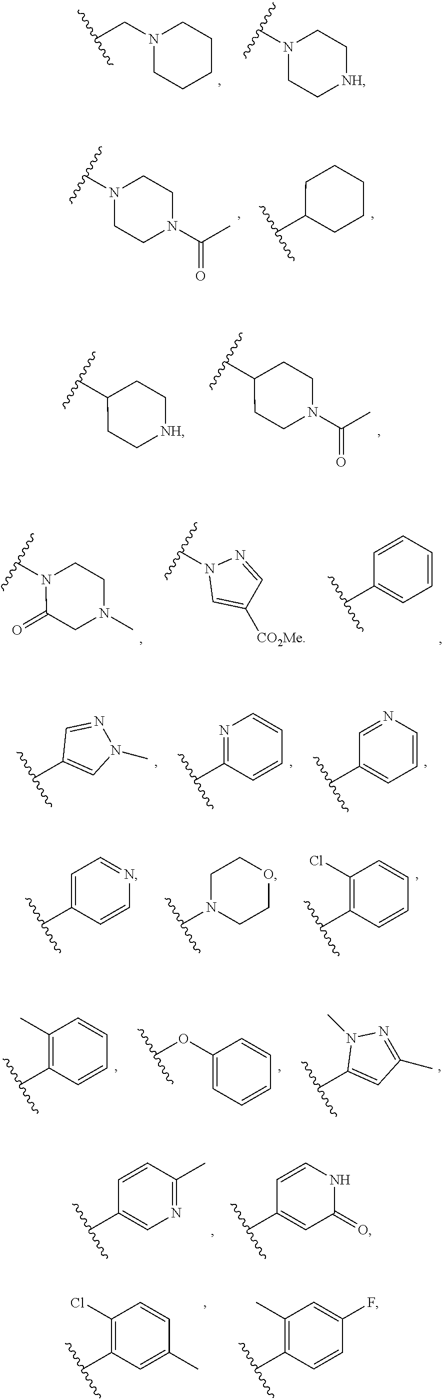

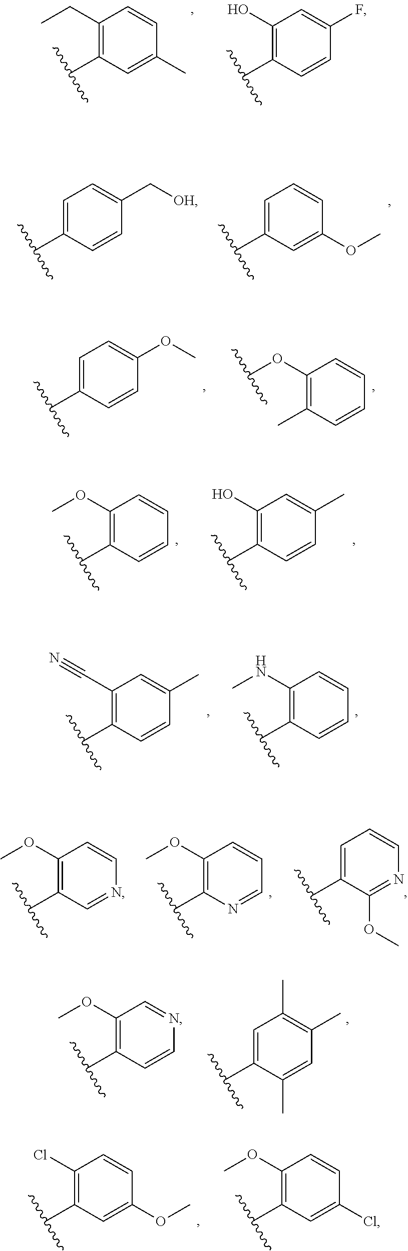

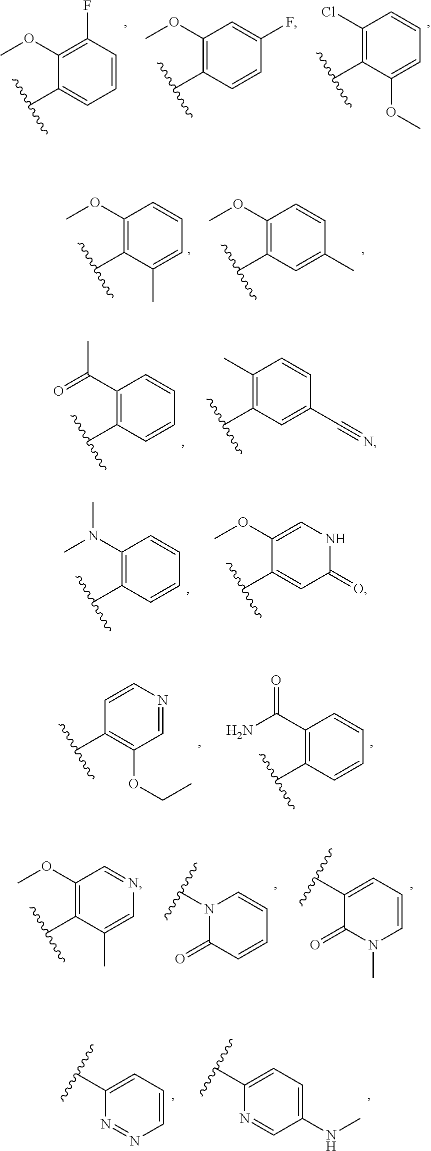

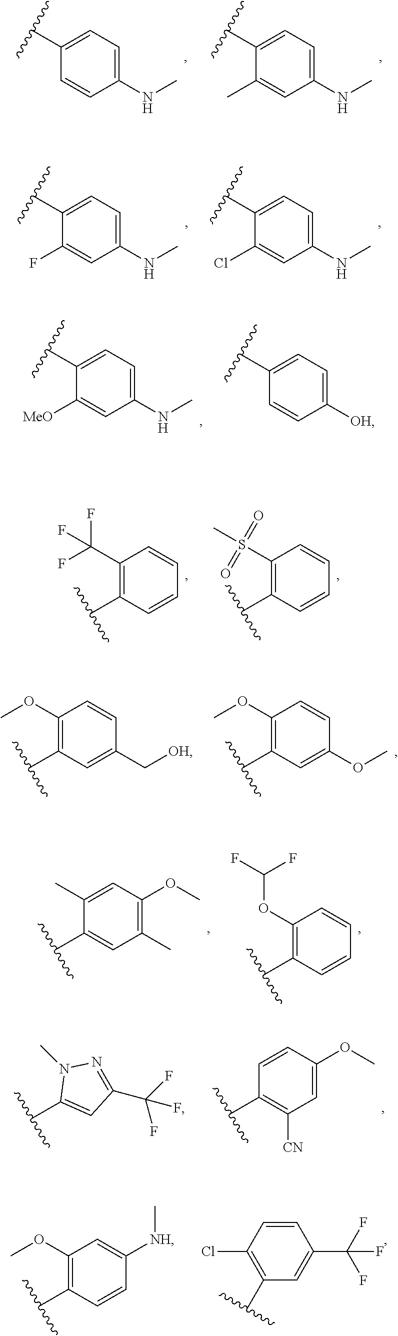

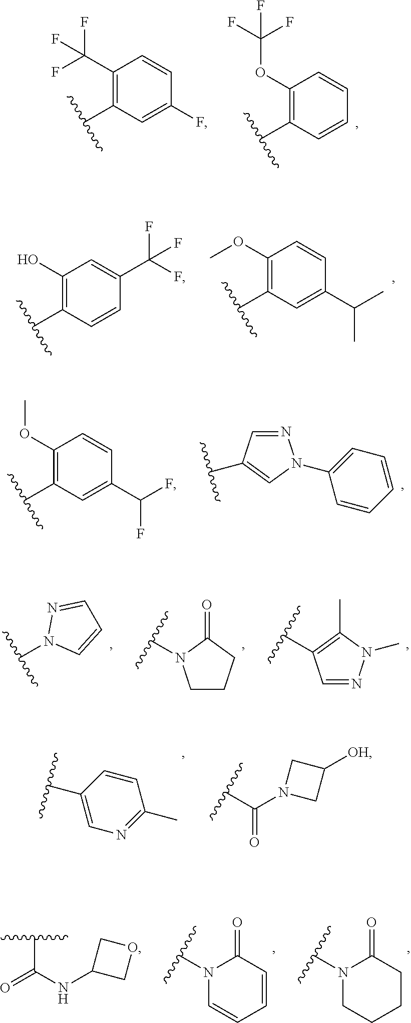

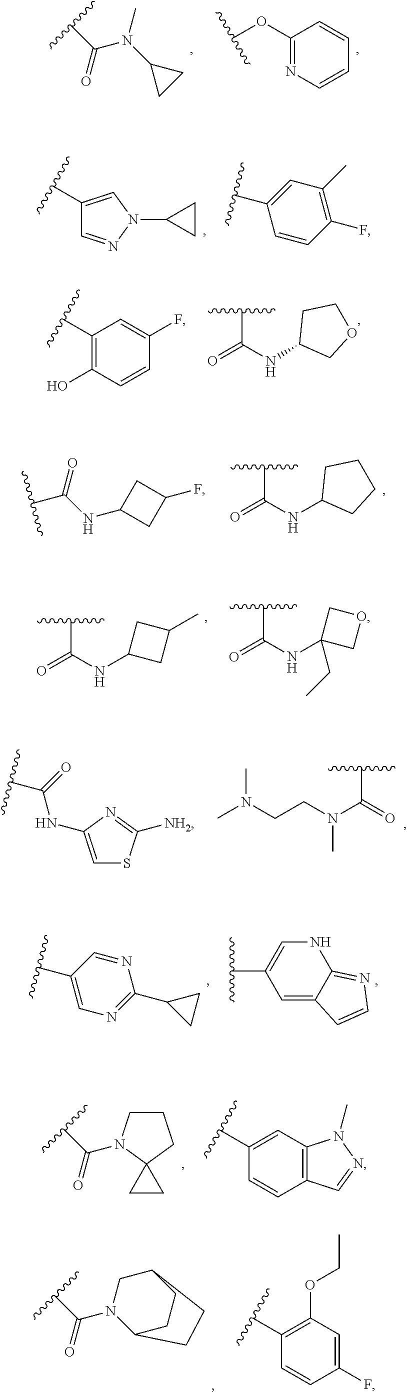

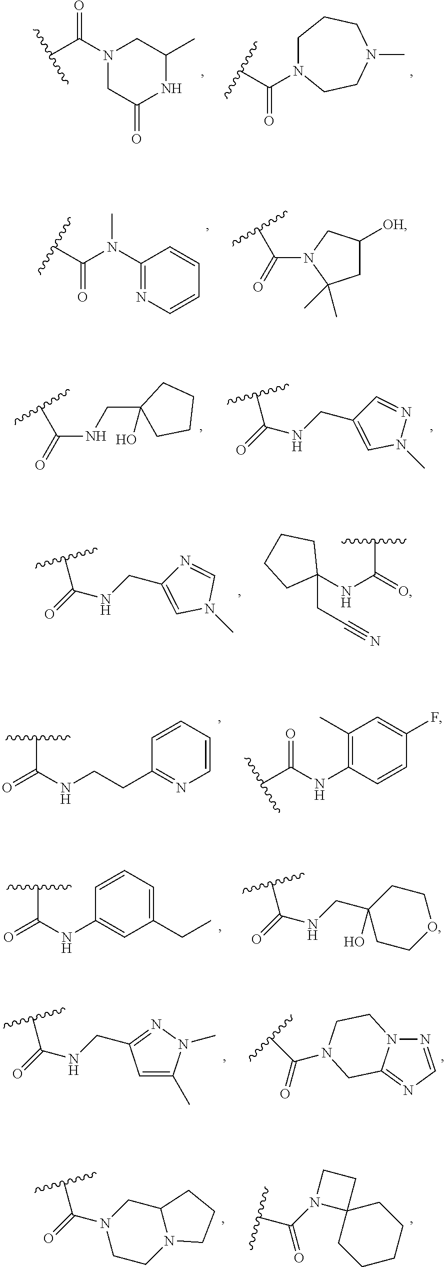

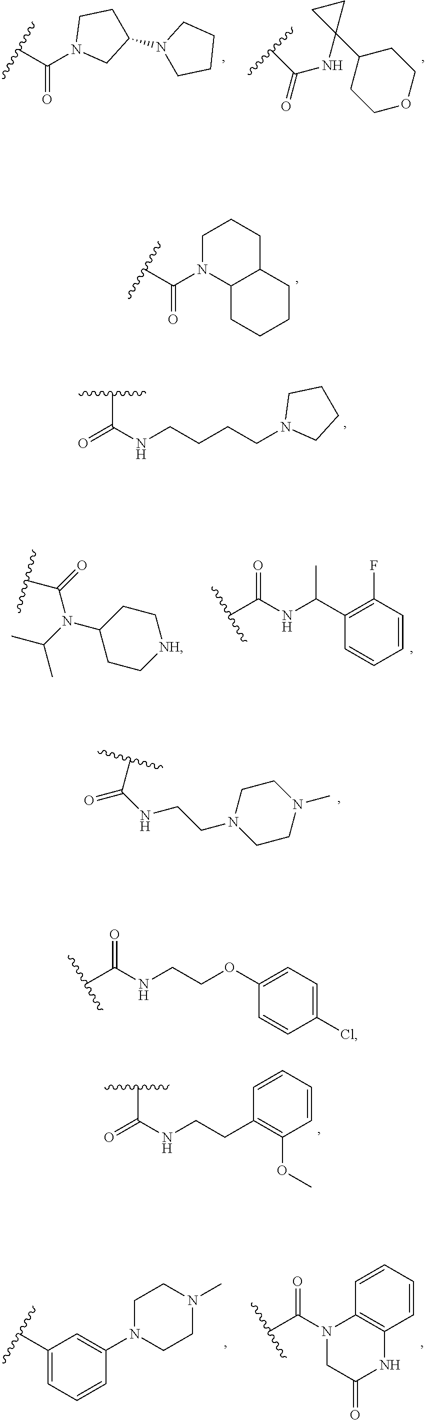

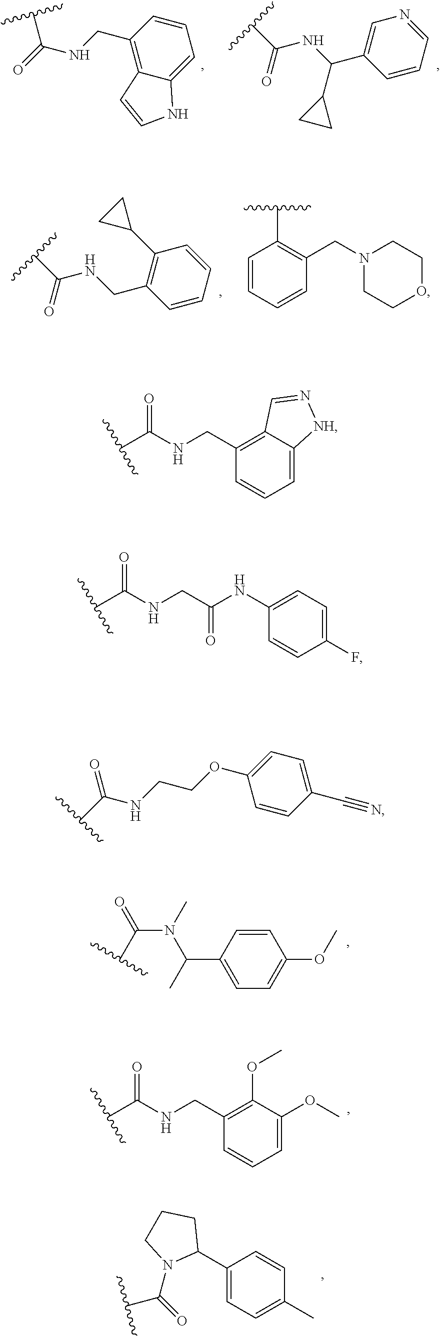

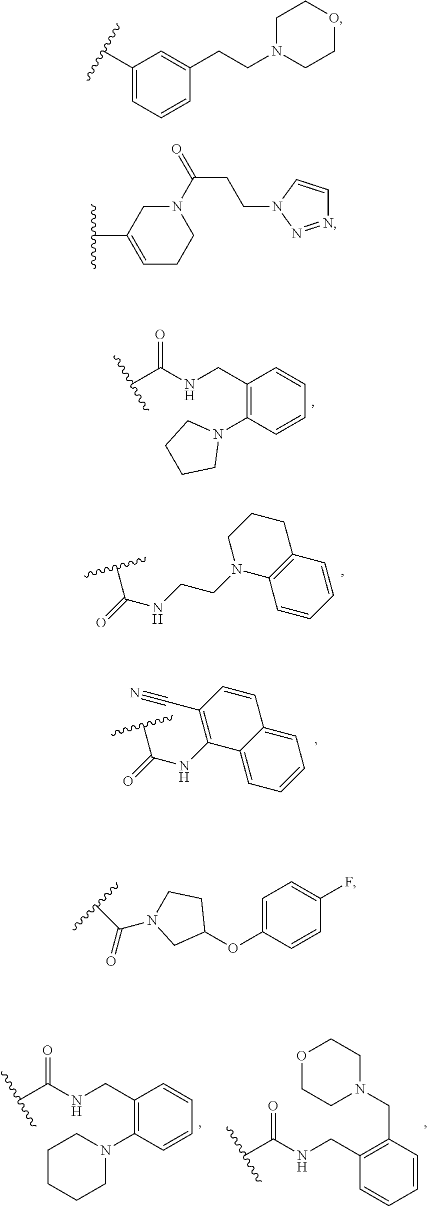

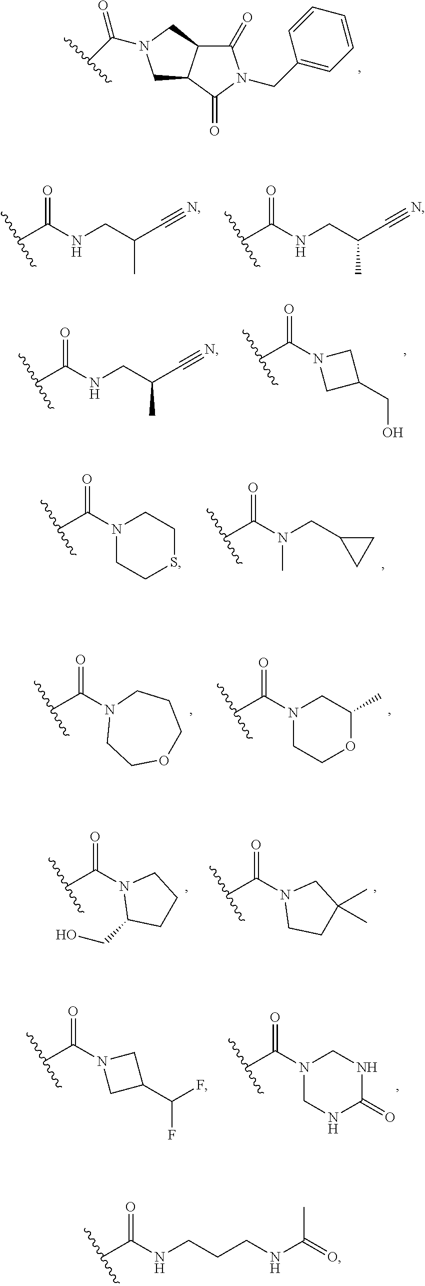

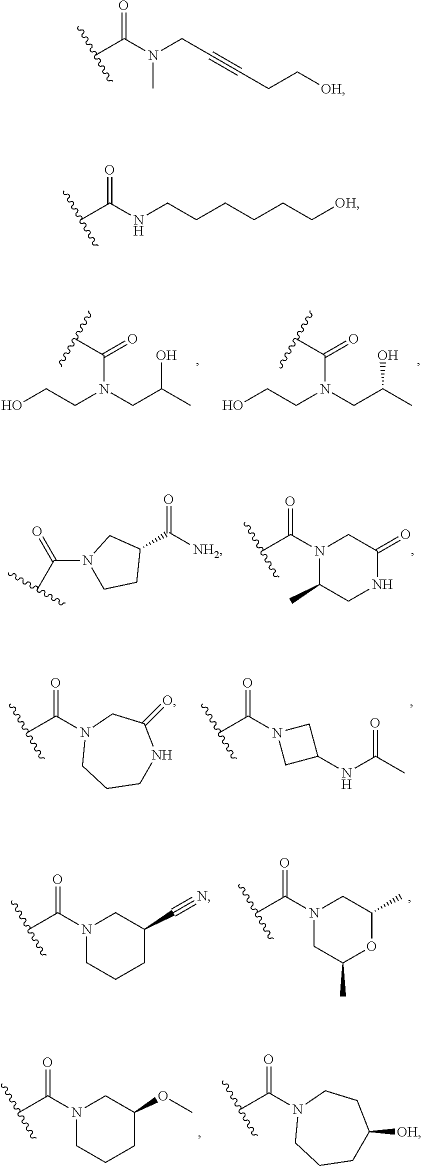

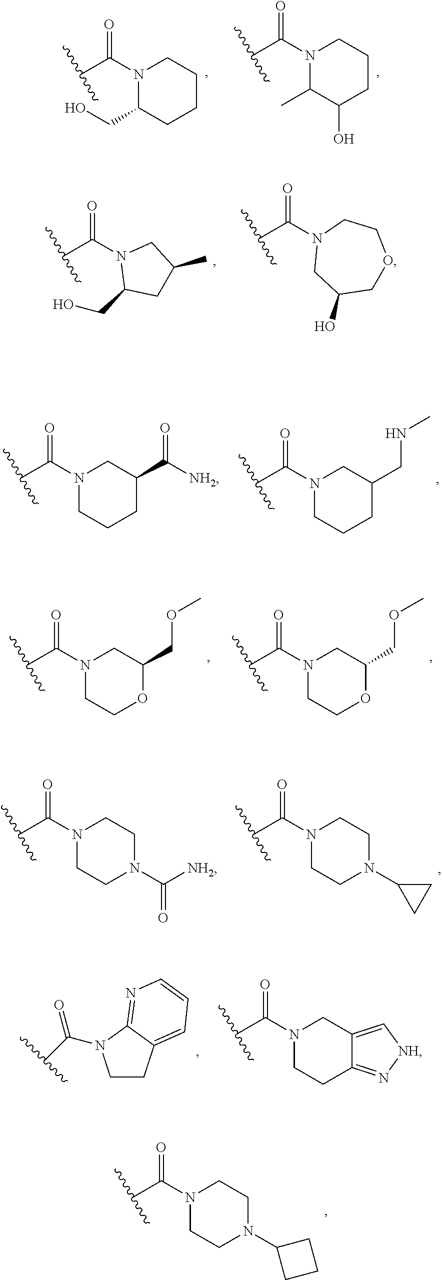

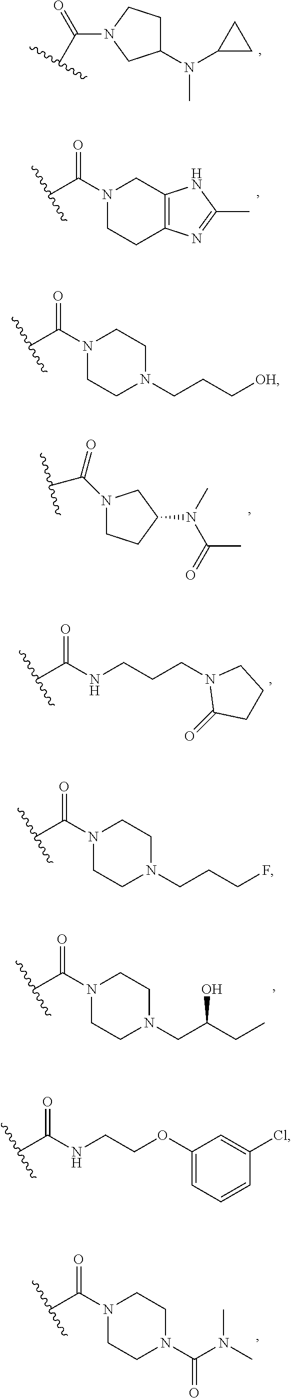

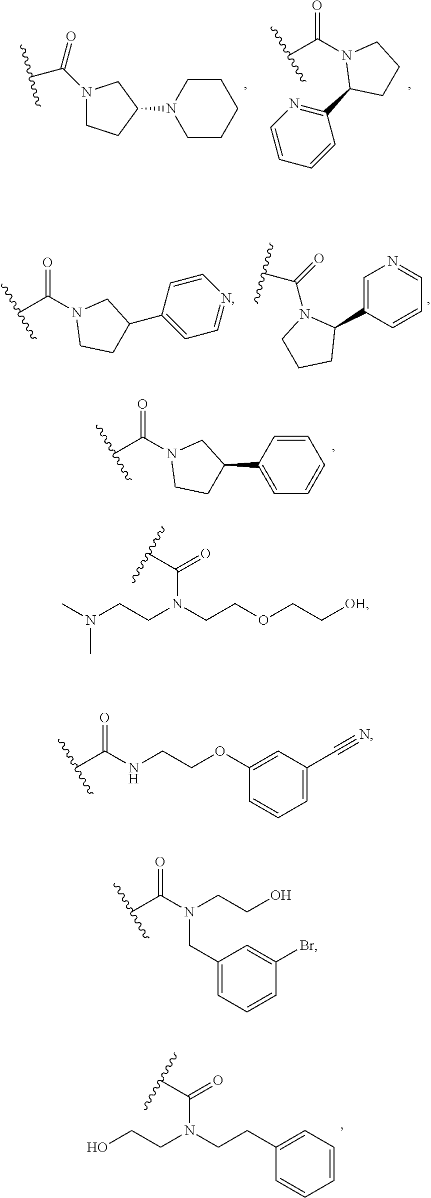

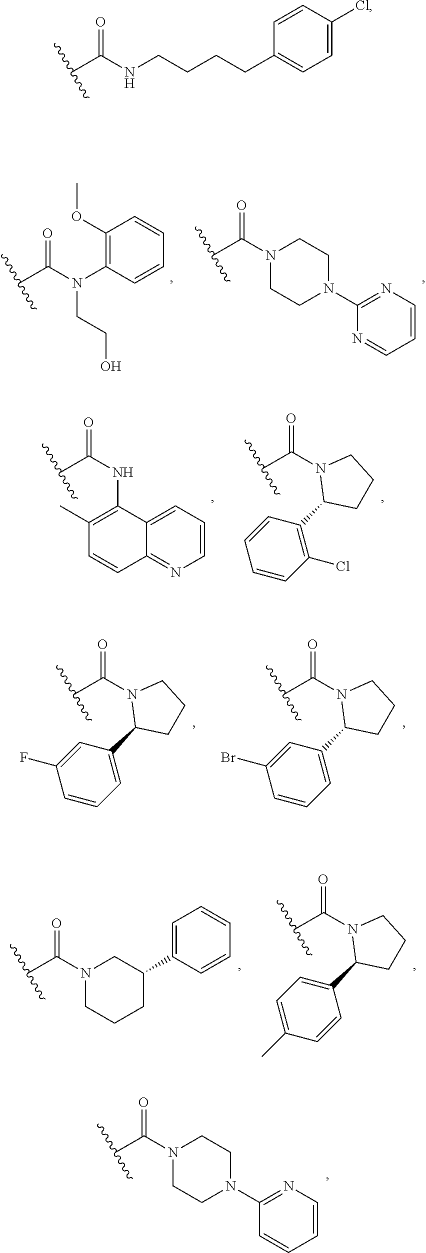

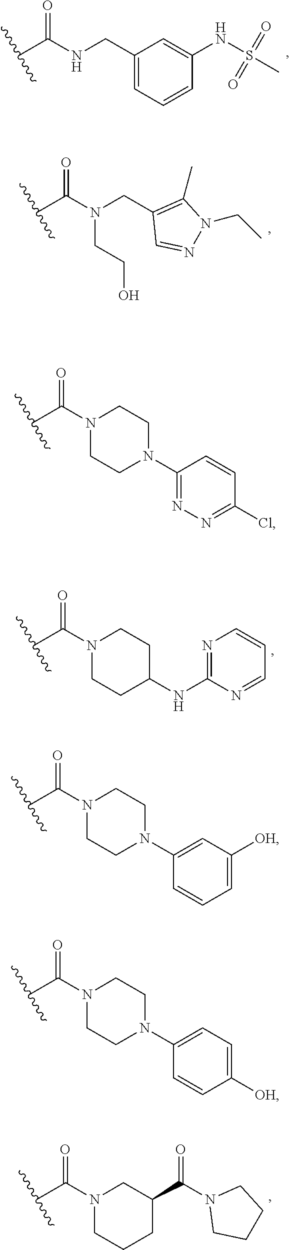

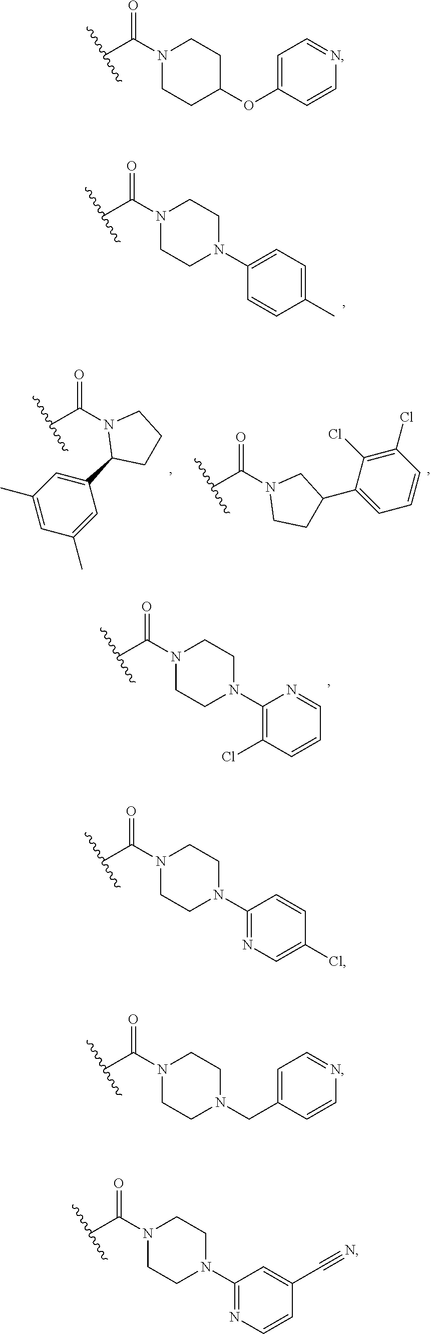

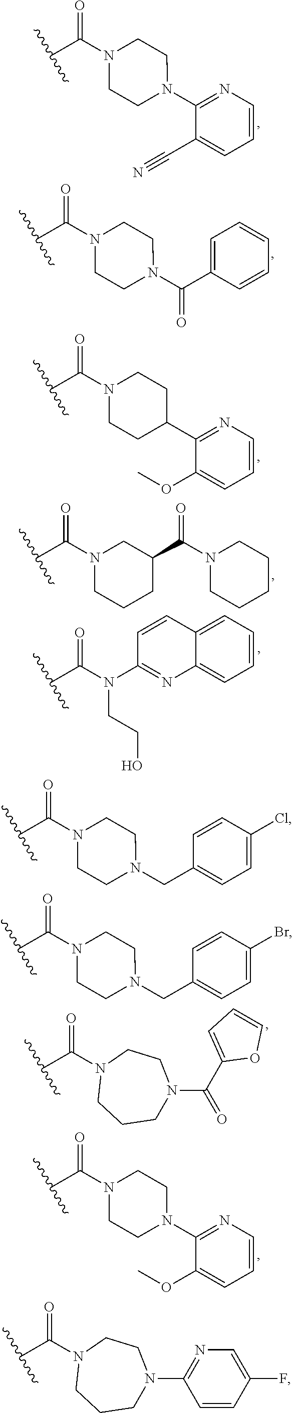

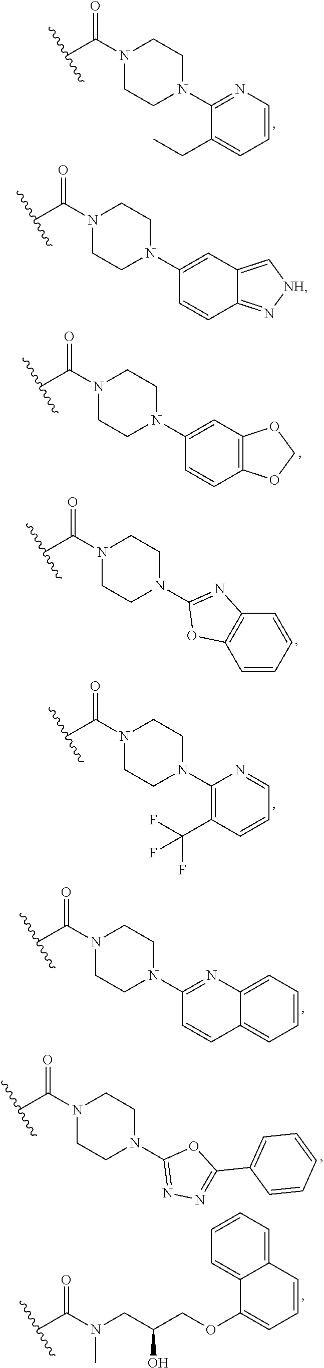

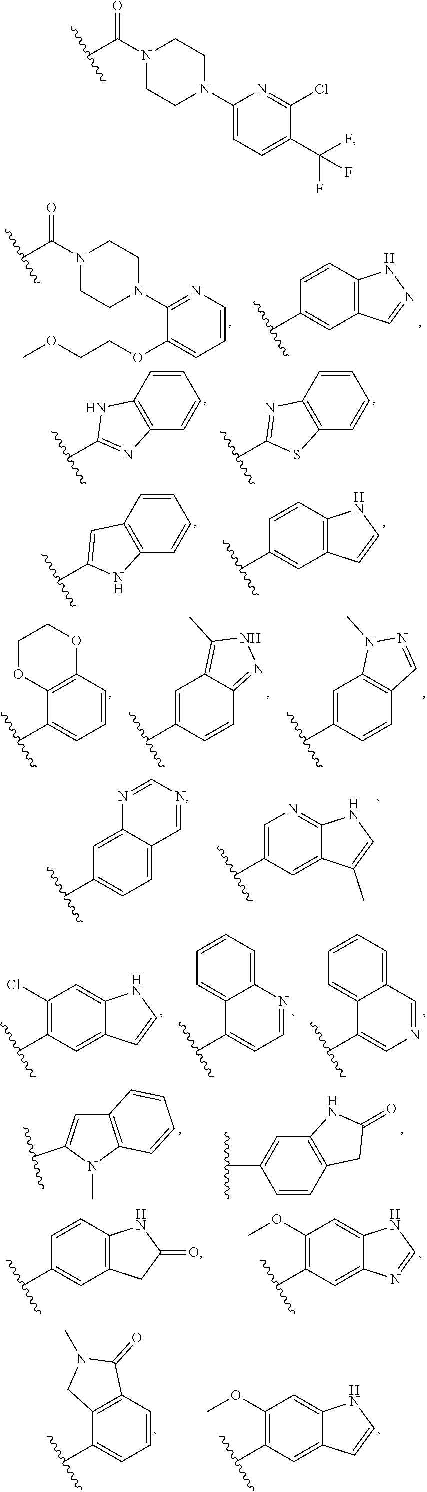

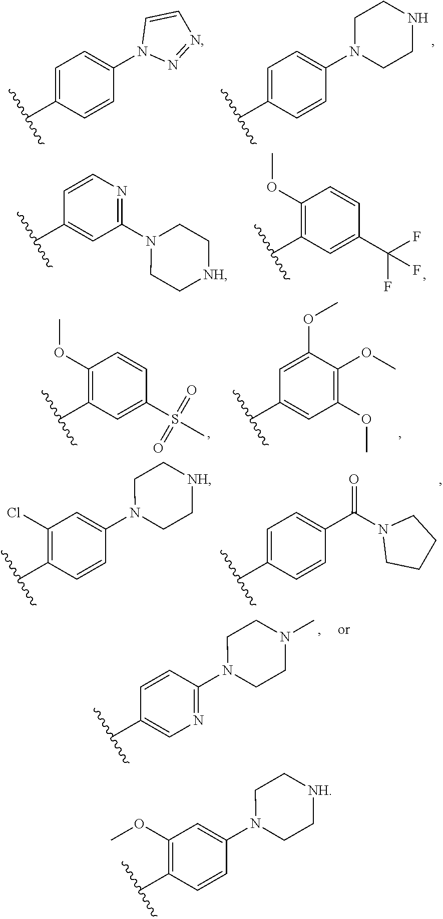

In some embodiments, R^{w} is

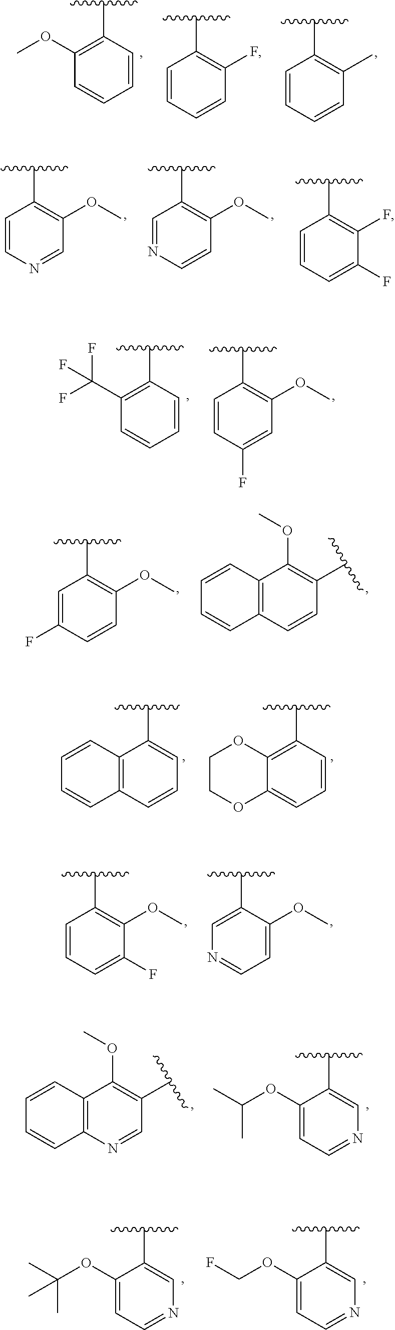

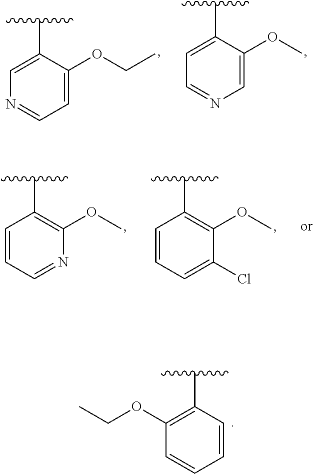

In some embodiments, R^{w} is —S(O)₂NH₂, —S(O)₂N(CH₃)₂, —S(O)(NH)CH₃,

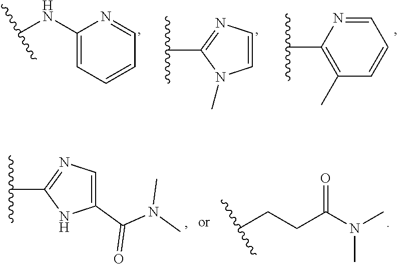

In some embodiments, R^{w} is

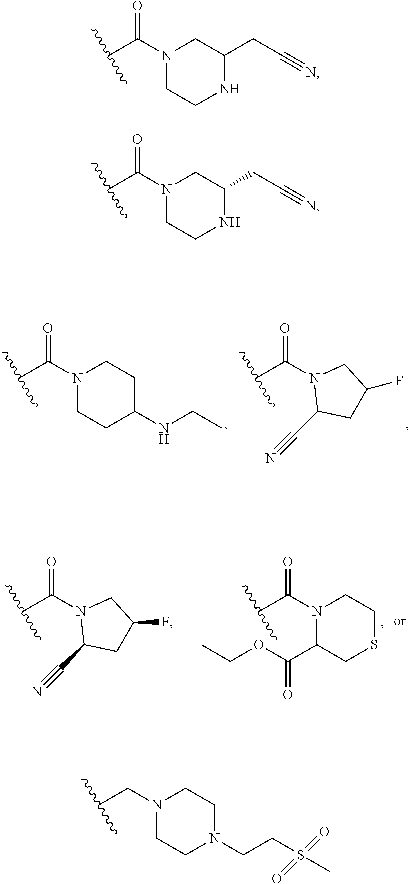

In some embodiments, R^{w} is

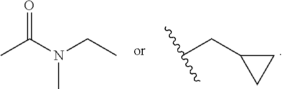

In some embodiments, R^{x} is selected from hydrogen, R^{A}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)N(R)₂, and —NRS(O)₂R.

In some embodiments, R^{x} is hydrogen. In some embodiments, R^{x} is R^{A}. In some embodiments, R^{x} is halogen. In some embodiments, R^{x} is —CN. In some embodiments, R^{x} is —NO₂. In some embodiments, R^{x} is —OR. In some embodiments, R^{x} is —SR. In some embodiments, R^{x} is —NR₂. In some embodiments, R^{x} is —SiR₃. In some embodiments, R is —S(O)₂R. In some embodiments, R^{x} is —S(O)₂NR₂. In some embodiments, R^{w} is —S(O)(NR)R. In some embodiments, R^{x} is —S(O)R. In some embodiments, R^{x} is —C(O)R. In some embodiments, R^{x} is —C(O)OR. In some embodiments, R^{w} is —C(O)NR₂. In some embodiments, R^{x} is —C(O)NROR. In some embodiments, R^{w} is —OC(O)R. In some embodiments, R^{x} is —OC(O)NR₂. In some embodiments, R^{x} is —P(O)R₂. In some embodiments, R^{w} is —P(O)(OR)₂. In some embodiments, R^{x} is —OP(O)R₂. In some embodiments, R^{x} is —OP(O)(OR)₂. In some embodiments, R^{x} is —OP(O)(OR)NR₂. In some embodiments, R^{x} is —OP(O)(NR₂)₂. In some embodiments, R^{x} is —NRC(O)OR. In some embodiments, R^{x} is —NRC(O)R. In some embodiments, R^{x} is —NRC(O)N(R)₂. In some embodiments, R^{x} is —NP(O)R₂. In some embodiments, R^{x} is —NRP(O)(OR)₂. In some embodiments, RN is —NRP(O)(OR)NR₂. In some embodiments, R^{x} is —NRP(O)(NR₂)₂. In some embodiments, R^{x} is —NRS(O)₂R.

In some embodiments, each R^{x} is independently selected from R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R

In some embodiments, R^{x} is optionally substituted C₁₋₆ aliphatic. In some embodiments, R^{x} is C₁₋₆ aliphatic optionally substituted with —OR^{o}, wherein R^{o} is hydrogen or C₁₋₆ aliphatic.

In some embodiments, R^{x} is —CO₂Me, —CH₂OH, —C(O)Me, —C(O)Et, —C(O)iPr, —C(O)cyclopropyl, —C(O)oxetanyl, —C(O)tetrahydropyranyl, or pyridyl.

In some embodiments, R^{x} is fluoro. In some embodiments, R is —S(O)₂CH₃. In some embodiments, R^{x} is —C(O)N(CH₃)₂.

In some embodiments, R^{x} is —C(O)R or —C(O)OR. In some embodiments, R^{X} is —C(O)R.

In some embodiments, R^{x} is —C(O)R, wherein R of R^{x} is optionally substituted C₁₋₆ aliphatic. In some embodiments, R is —C(O)R, wherein R of R is C₁₋₆ aliphatic. In some embodiments, R^{w} is —C(O)R, wherein R of R is methyl, ethyl, n-propyl, or isopropyl. In some embodiments, R^{x} is —C(O)R, wherein R of R is C₁₋₆ aliphatic substituted with —OR^{o} or —C(O)OR^{o}, wherein R^{o} is hydrogen or C₁₋₆ aliphatic. In some embodiments, RN is —C(O)R, wherein R of R is C₁₋₆ aliphatic substituted with —OR^{o}, wherein R^{o} is hydrogen or C₁₋₆ aliphatic. In some embodiments, R^{x} is —C(O)R, wherein R of R is C₁₋₆ aliphatic substituted with —C(O)OR^{o}, wherein R^{o} is hydrogen or C₁₋₆ aliphatic.

In some embodiments, R^{w} is

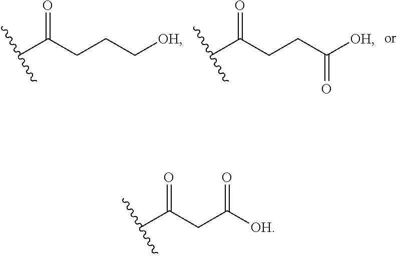 [SMILES: *C(=O)CCCO ; *C(=O)CCC(=O)O ; "or" ; *C(=O)CC(=O)O]

In some embodiments, R^{x} is —C(O)R, wherein R of R^{X} is C₁₋₆ aliphatic optionally substituted with —C(O)OR^{o}, —OR^{o}, halogen (e.g., fluoro), or ═O. In some embodiments, R^{x} is —C(O)OR. In some embodiments, R^{X} is —C(O)OR, wherein R of R^{X} is an optionally substituted C₁₋₆ aliphatic.

In some embodiments, R^{x} is

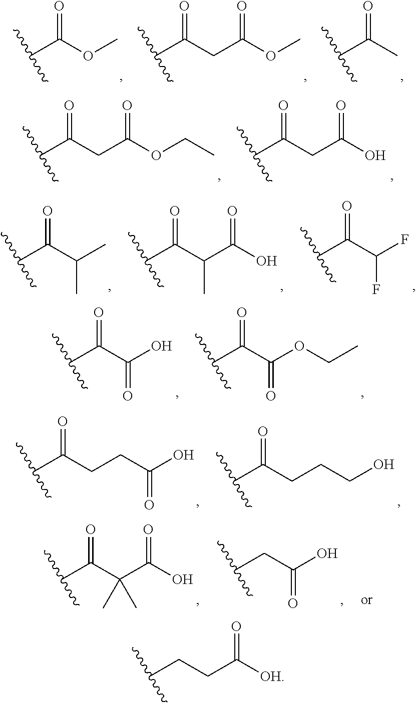

In some embodiments, each R^{y} is independently selected from R^{A}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{y} is hydrogen. In some embodiments, R^{y} is R^{A}. In some embodiments, R^{y} is halogen. In some embodiments, R^{y} is —CN. In some embodiments, R^{y} is —NO₂. In some embodiments, R^{y} is —OR. In some embodiments, R^{y} is —SR. In some embodiments, R^{y} is —NR₂. In some embodiments, R^{y} is —SiR₃. In some embodiments, R^{y} is —S(O)₂R. In some embodiments, R^{y} is —S(O)₂NR₂. In some embodiments, R^{y} is —S(O)(NR)R. In some embodiments, R^{y} is —S(O)R. In some embodiments, R^{y} is —C(O)R. In some embodiments, R^{y} is —C(O)OR. In some embodiments, R^{y} is —C(O)NR₂. In some embodiments, R^{y} is —C(O)NROR. In some embodiments, R^{y} is —OC(O)R. In some embodiments, R^{x} is —OC(O)NR₂. In some embodiments, R^{y} is —P(O)R₂. In some embodiments, R^{y} is —P(O)(OR)₂. In some embodiments, R^{y} is —OP(O)R₂. In some embodiments, R^{y} is —OP(O)(OR)₂. In some embodiments, R^{y} is —OP(O)(OR)NR₂. In some embodiments, R^{y} is —OP(O)(NR₂)₂. In some embodiments, R^{y} is —NRC(O)OR. In some embodiments, R^{y} is —NRC(O)R. In some embodiments, R^{y} is —NRC(O)N(R)₂. In some embodiments, R^{y} is —NP(O)R₂. In some embodiments, R^{y} is —NRP(O)(OR)₂. In some embodiments, R^{y} is —NRP(O)(OR)NR₂. In some embodiments, R^{y} is —NRP(O)(NR₂)₂. In some embodiments, R^{y} is —NRS(O)₂R.

In some embodiments, R^{y} is fluoro, chloro, bromo, iodo, methyl, ethyl, cyclopropyl, —CF₃, —CN, CH₂O, —CO₂H, —CO₂Me, —CO₂tBu, —C(O)Me, —NH₂, —NHMe, —NHAc, —NHC(O)Et, —OH, —OMe, —OCH₂CH₂NH₂, —CH₂OH, —CH₂OMe, —CH₂NHMe, —CH₂NHAc, —CH₂SO₂Me, —SO₂Me, —SO₂NH₂, —SO₂NHMe,

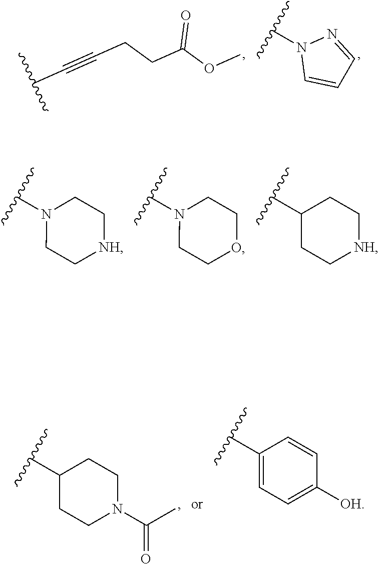

In some embodiments, R^{Y} is —C(O)H.

In some embodiment, R^{w}, R^{x}, and R^{y} are as depicted in the compounds of Table 1A, below.

As described above and defined herein, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic heteroaryl ring having 1-4 heteroatoms independently selected from nitrogen, oxygen, and sulfur.

In some embodiments, each R^{A} is independently selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, a 3-7 membered saturated heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic saturated heterocyclic ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, a 5-10 membered monocyclic or bicyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or a monocyclic heteroaryl ring having 1-4 heteroatoms independently selected from nitrogen, oxygen, and sulfur.

In some embodiments, each R^{A} is independently an optionally substituted group selected from C₁₋₆ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments, R^{A} is an optionally substituted R^{A} is a 5-10 membered monocyclic or bicyclic heteroaryl ring having 1-4 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is optionally substituted naphthalenyl. In some embodiments, R^{A} is optionally substituted dihydrobenzo[b][1,4]dioxinyl.

In some embodiments, R^{A} is an optionally substituted 8-11 membered monocyclic or bicyclic aryl or heteroaryl with 1-4 heteroatoms independently selected from nitrogen, oxygen and sulfur. In some embodiments, R^{A} is optionally substituted naphthalenyl or dihydrobenzo[b][1,4]dioxinyl. In some embodiments, R^{A} is optionally substituted quinolinyl.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1A, below.

As described above and defined herein, each R^{B′} is independently -L^{B}-Cy^{B1}-H or -L^{B}-Cy^{B1}-Cy^{B2}.

In some embodiments, R^{B′} is -L^{B}-Cy^{B1}-H. In some embodiments, R^{B′} is -L^{B}-Cy^{B1}-Cy^{B2}.

In some embodiments, R^{B′} is

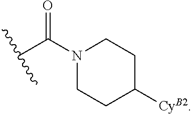

In some embodiments, R^{B′} is

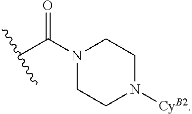

In some embodiments, R^{B′} is

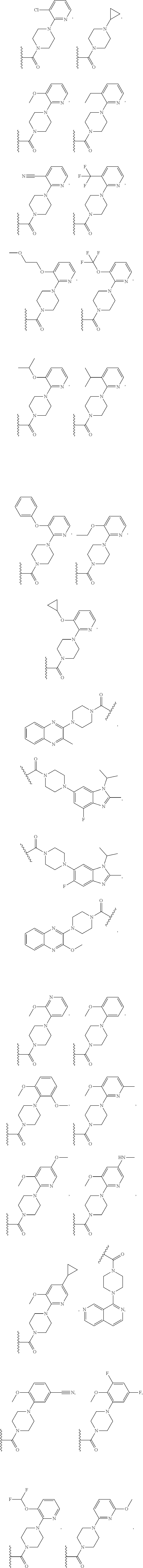

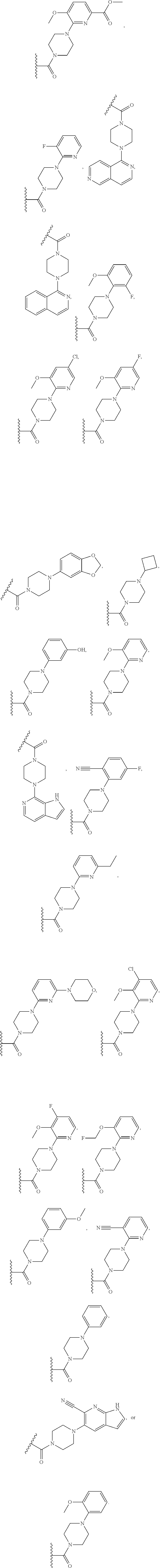

As described above and defined herein, each L^{B} is independently a covalent bond or a Cis bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—

In some embodiments, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—.

In some embodiments, L^{B} is a covalent bond. In some embodiments, L^{B} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—. In some embodiments, L^{B} is a C₁₋₃ bivalent straight or branched saturated or unsaturated hydrocarbon chain wherein 1-2 methylene units of the chain are independently and optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1-2 methylene units of the chain are independently and optionally replaced with —O—, —C(O)—, or —NR—. In some embodiments, L^{B} is a C₁₋₃ bivalent straight or branched saturated or unsaturated hydrocarbon chain wherein 1 methylene unit of the chain is optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1 methylene unit of the chain is optionally replaced with —O—, —C(O)—, or —NR—. In some embodiments, L^{B} is a C₁₋₃ bivalent straight or branched saturated or unsaturated hydrocarbon chain wherein 1 methylene unit of the chain is replaced with —C(O)—. In some embodiments, L^{B} is —C(O)—.

In some embodiments, L^{B} is a C₁₋₃ bivalent straight or branched saturated or unsaturated hydrocarbon chain. In some embodiments, L^{B} is —CH₂—.

In some embodiments, L^{B} is —C(O)—, —C(S)—, —C(NR)R—, —S(O)—, —S(O)₂— or —S(O)(NR)—. In some embodiments, L^{B} is —C(O)—, —C(S)—, or —C(NR)R—. In some embodiments, L^{B} is —C(O)—, —S(O)—, —S(O)₂— or —S(O)(NR)—. In some embodiments, L^{B} is —C(S)—. In some embodiments, L^{B} is —C(NR)R—. In some embodiments, L^{B} is —S(O)—. In some embodiments, L^{B} is —S(O)₂—. In some embodiments, L^{B} is —S(O)(NR)—.

As described above and defined herein, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

It will be appreciated that all embodiments to Cy^{B1} may refer to a terminal ring (or otherwise optionally substituted ring) in structures with -L^{B}-Cy^{B1}, or a ring further connected to Cy^{B2} as in structures -L^{B}-Cy^{B}-Cy^{B2}, regardless of how presented. By way of example, an embodiment to Cy^{B1} is phenylenyl, refers to phenylenyl in -L^{B}-Cy^{B1}-Cy^{B2}, and phenyl in -L^{B}-Cy^{B1}. Similarly, an embodiment Cy^{B1} is

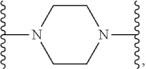 [SMILES: *N1CCN(*)CC1]

refers to

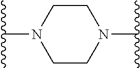 [SMILES: *N1CCN(*)CC1]

in -L^{B}-Cy^{B1}-Cy^{B2}, and

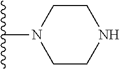 [SMILES: *N1CCNCC1]

in -L^{B}-Cy^{B1}. Similarly, an embodiment to Cy^{B1} is

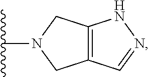 [SMILES: *N1Cc2cn[nH]c2C1]

refers to

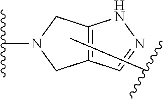

in -L^{B}-Cy^{B1}-Cy^{B2}, and

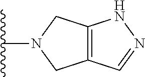 [SMILES: *N1Cc2cn[nH]c2C1]

in -L^{B}-Cy^{B1}.

In some embodiments, each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

In some embodiments, Cy^{B1} is optionally substituted phenylenyl. In some embodiments, Cy^{B1} is phenylenyl.

In some embodiments, Cy^{B1} is optionally substituted 3-7 membered saturated or partially unsaturated carbocyclylenyl or heterocyclylenyl having 1-4 heteroatoms independently selected from nitrogen, oxygen, and sulfur. In some embodiments, Cy^{B1} is optionally substituted 5-10 membered monocyclic or bicyclic arylenyl or heteroarylenyl having 1-4 heteroatoms independently selected from nitrogen, oxygen, and sulfur.

In some embodiments, Cy^{B1} is optionally substituted 3-7 membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-2 heteroatoms independently selected from nitrogen, oxygen, and sulfur. In some embodiments, Cy^{B1} is optionally substituted 6-membered saturated or partially unsaturated heterocyclylenyl having 1-2 nitrogen heteroatoms. In some embodiments, Cy^{B1} is optionally substituted piperadinylenyl or piperazinylenyl.

In some embodiments, Cy^{B1} is a 3-7 membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-2 heteroatoms independently selected from nitrogen, oxygen, and sulfur. In some embodiments, Cy^{B1} is a 6-membered saturated or partially unsaturated heterocyclylenyl having 1-2 nitrogen heteroatoms. In some embodiments, Cy^{B1} is a piperadinylenyl or piperazinylenyl. In some embodiments, Cy^{B1} is

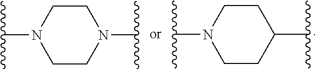

In some embodiments, Cy^{B1} is an optionally substituted piperidinonyl or piperazinonyl. In some embodiments, Cy^{B1} is an optionally substituted dihydropyridinyl. In some embodiments, Cy^{B1} is an optionally substituted thiomorpholinyl.

In some embodiments, Cy^{B1} is a 3-10 membered saturated or partially unsaturated monocyclic or bicyclic heterocyclylenyl having 1-4 heteroatoms independently selected from nitrogen, oxygen, and sulfur.

In some embodiments, Cy^{B1} is an optionally substituted 8-10 membered saturated or partially unsaturated bicyclic heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-2 heteroatoms independently selected from nitrogen, oxygen, and sulfur. In some embodiments, Cy^{B1} is an optionally substituted 8-membered saturated or partially unsaturated heterocyclylenyl having 1-2 nitrogen heteroatoms. In some embodiments, Cy^{B1} is optionally substituted

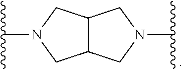 [SMILES: *N1CC2CN(*)CC2C1]

In some embodiments, Cy^{B1} is an optionally substituted

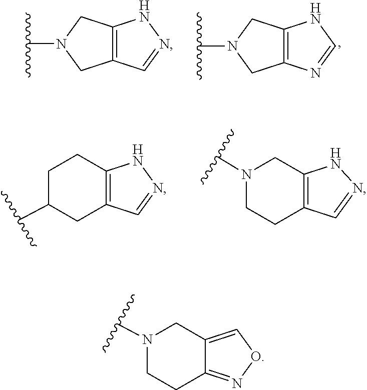

In some embodiments, Cy^{B1} is an optionally substituted 5-6 membered monocyclic heteroarylenyl having 1-4 heteroatoms independently selected from nitrogen, oxygen, and sulfur. In some embodiments, Cy^{B1} is an optionally substituted 6 membered monocyclic heteroarylenyl having 1-4 heteroatoms independently selected from nitrogen, oxygen, and sulfur. In some embodiments, Cy^{B1} is an optionally substituted pyridinonyl, pyrazinonyl, or pyrimidinonyl.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, Cy^{B2} is optionally substituted phenyl. In some embodiments, Cy^{B2} is phenyl. In some embodiments, Cy^{B2} is phenyl, optionally substituted with —CN, halogen, —R^{o}, or —OR^{o}, wherein R^{o} is hydrogen or C₁₋₆ aliphatic.

In some embodiments, Cy^{B2} is optionally substituted 3-7 membered saturated or partially unsaturated carbocyclic or heterocyclic ring having 1-2 heteroatoms independently selected from nitrogen, oxygen, and sulfur.

In some embodiments, Cy^{B2} is optionally substituted 3-7 membered saturated or partially unsaturated carbocyclic. In some embodiments, Cy^{B2} is optionally substituted cyclopropyl. In some embodiments, Cy^{B2} is cyclopropyl. In some embodiments, Cy^{B2} is optionally substituted cyclobutyl. In some embodiments, Cy^{B2} is cyclobutyl.

In some embodiments, Cy^{B2} is optionally substituted cyclopentyl. In some embodiments, Cy^{B2} is cyclopentyl. In some embodiments, Cy^{B2} is optionally substituted cyclohexyl. In some embodiments, Cy^{B2} is cyclohexyl.

In some embodiments, Cy^{B2} is optionally substituted 5-10 membered monocyclic or bicyclic aryl or heteroaryl with 1-4 heteroatoms independently selected from nitrogen, oxygen and sulfur.

In some embodiments, Cy^{B2} is optionally substituted 5-6 membered monocyclic heteroaryl with 1-4 heteroatoms independently selected from nitrogen, oxygen and sulfur. In some embodiments, Cy^{B2} is optionally substituted 5-6 membered monocyclic heteroaryl with 1-2 heteroatoms independently selected from nitrogen, oxygen and sulfur. In some embodiments, Cy^{B2} is optionally substituted pyridinyl. In some embodiments, Cy^{B2} is pyridinyl, optionally substituted with —CN, halogen, —R^{o}, —OR^{o}, —N(R^{o})₂—C(O)OR^{o}, wherein each R^{o} is independently hydrogen; C₁₋₆ aliphatic, which may be optionally substituted with halogen, —(CH₂)₀₋₂OH, or —(CH₂)₀₋₂OR^{•}, where R^{•} is C₁₋₄ aliphatic; or a 5-6-membered saturated, partially unsaturated, or aryl ring having 0-4 heteroatoms independently selected from nitrogen, oxygen, or sulfur (e.g., phenyl or morpholinyl).

In some embodiments, Cy^{B2} is optionally substituted 6 membered monocyclic heteroaryl with 1-2 heteroatoms independently selected from nitrogen, oxygen and sulfur. In some embodiments, Cy^{B2} is optionally substituted pyridinyl, pyrimidinyl, pyridazinyl, pyrazinyl, or triazinyl.

In some embodiments, Cy^{B2} is optionally substituted optionally substituted pyridinonyl, pyrazinonyl, or pyrimidinonyl

In some embodiments, Cy^{B2} is optionally substituted 5 membered monocyclic heteroaryl with 1-2 heteroatoms independently selected from nitrogen, oxygen and sulfur. In some embodiments, Cy^{B2} is optionally substituted pyrazolyl, imidazolyl, or triazolyl.

In some embodiments, Cy^{B2} is optionally substituted 8-10 membered bicyclic aryl or heteroaryl with 1-4 heteroatoms independently selected from nitrogen, oxygen and sulfur.

In some embodiments, Cy^{B2} is optionally substituted 9-membered bicyclic heteroaryl with 1-4 heteroatoms independently selected from nitrogen, oxygen and sulfur. In some embodiments, Cy^{B2} is optionally substituted 9-membered bicyclic heteroaryl with 1-4 nitrogen heteroatoms. In some embodiments, Cy^{B2} is optionally substituted benzimidazolyl, indazolyl, or azaindolyl (e.g., pyrrolo[2,3-c]pyridinyl or pyrrolo]2,3-b]pyridinyl). In some embodiments, Cy^{B2} is benzimidazolyl indazolyl, or azaindolyl (e.g., pyrrolo[2,3-c]pyridinyl or pyrrolo]2,3-b]pyridinyl) optionally substituted with —CN, halogen or —R^{o}, wherein R^{o} is hydrogen or C₁₋₆ aliphatic. In some embodiments, Cy^{B2} is optionally substituted indolyl or azaindolyl.

In some embodiments, Cy^{B2} is optionally substituted 10-membered bicyclic aryl or heteroaryl with 1-4 heteroatoms independently selected from nitrogen, oxygen and sulfur. In some embodiments, Cy^{B2} is optionally substituted 10-membered bicyclic heteroaryl with 1-4 nitrogen heteroatoms. In some embodiments, Cy^{B2} is optionally substituted quinoxalinyl, isoquinolinyl, 2,6-naphthyridinyl, or 2,7-naphthyridinyl. In some embodiments, Cy^{B2} is quinoxalinyl, isoquinolinyl, 2,6-naphthyridinyl, or 2,7-naphthyridinyl, optionally substituted with —R^{o}, wherein R^{o} is hydrogen or C₁₋₆ aliphatic.

In some embodiments, Cy^{B2} is optionally substituted naphthalenyl. In some embodiments, Cy^{B2} is naphthalenyl, optionally substituted with —R^{o} or —OR^{o}, wherein R^{o} is hydrogen or C₁₋₆ aliphatic.

In some embodiments, Cy^{B2} is optionally substituted benzo[d][1,3]dioxolyl.

In some embodiments, Cy^{B2} is

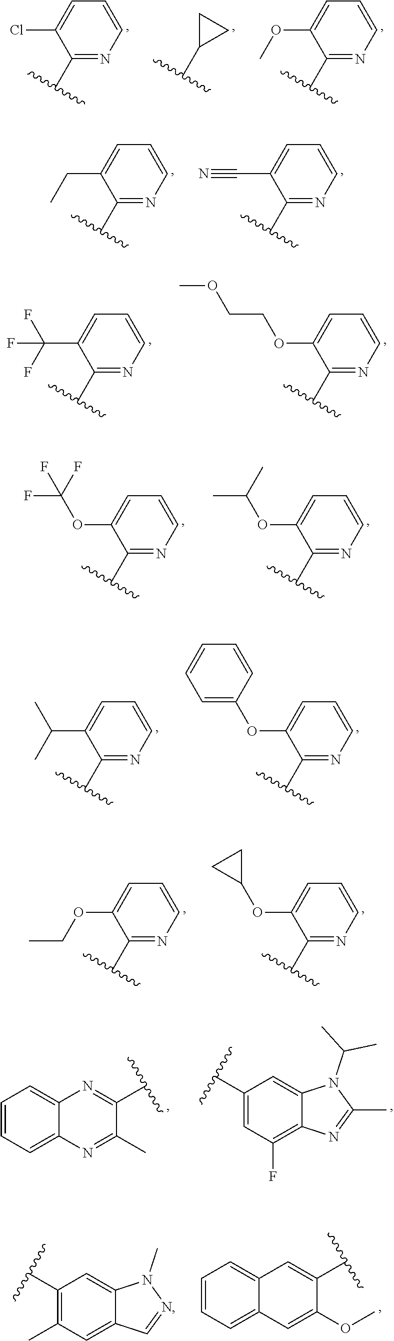

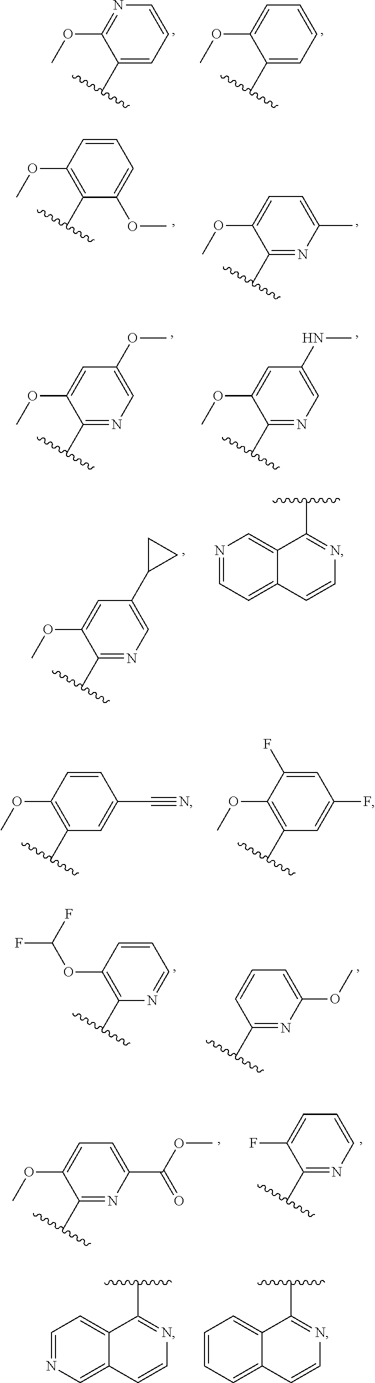

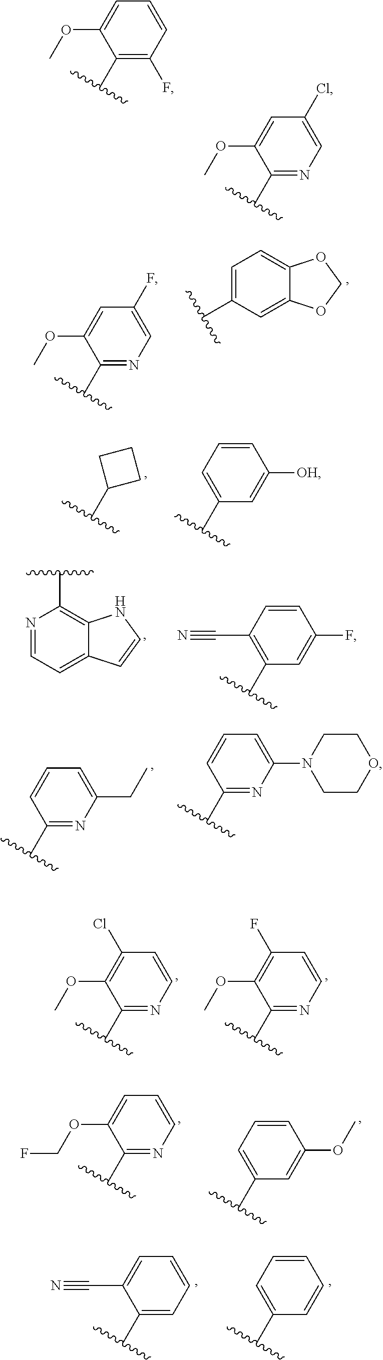

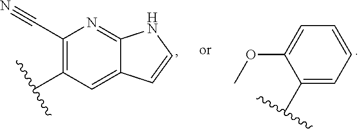

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur. In some embodiments, two R groups on adjacent atoms are optionally taken together with their intervening atoms to form a 3-7 membered saturated or partially unsaturated ring having 0-3 heteroatoms, in addition to the adjacent atoms to which they are attached, independently selected from nitrogen, oxygen, and sulfur.

In some embodiment, R is as depicted in the compounds of Table 1A, below.

As described above and defined herein, L^{x} is a covalent bond or an optionally substituted C₁₋₅ bivalent straight or branched saturated or unsaturated hydrocarbon chain wherein 1-2 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—.

In some embodiments, L^{x} is a covalent bond or an optionally substituted C₁₋₅ bivalent straight or branched saturated or unsaturated hydrocarbon chain wherein 1-2 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is a covalent bond or an optionally substituted C₁₋₅ bivalent straight or branched saturated or unsaturated hydrocarbon chain wherein 1-2 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— —S(O)(NR)—, or —CR═CR—.

In some embodiments, L^{x} is an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is an optionally substituted C₁₋₄ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is an optionally substituted C₁₋₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is an optionally substituted C₃ bivalent straight or branched saturated or unsaturated hydrocarbon chain wherein 1-3 methylene units of the chain are independently and optionally replaced with -Cy^{X}-, —O—, —C(O)—, —C(S)—, —CR₂—, —CF₂—, —CRF—, —CR(OR)—, —NR—, —S—, —S(O)—, —S(O)₂— or —CR═CR—.

In some embodiments, L^{X} is a covalent bond. In some embodiments, L^{x} is a C₁₋₅ bivalent straight or branched saturated or unsaturated hydrocarbon chain wherein 1-3 methylene units of the chain are independently and optionally replaced with -Cy^{X}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is a C₁₋₄ bivalent straight or branched saturated or unsaturated hydrocarbon chain wherein 1-3 methylene units of the chain are independently and optionally replaced with -Cy^{X}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is a C₁₋₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{x} is a C₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—.

In some embodiments, L^{x} is an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{x} is an optionally substituted C₁₋₅ bivalent straight or branched saturated or unsaturated hydrocarbon chain wherein 1-2 methylene units of the chain are independently and optionally replaced with —O—, —C(O)—, or —NR—.

In some embodiments, L^{x} is an optionally substituted C₁₋₅ bivalent straight or branched saturated or unsaturated hydrocarbon chain wherein 1-2 methylene units of the chain are independently and optionally replaced with —C(O)—, —C(S)—, —CR₂—, —CF2-, —CRF—, —CR(OR)—, —S—, —S(O)—, or —S(O)₂. In some embodiments, L^{x} is a C₁₋₅ bivalent straight or branched saturated or unsaturated hydrocarbon chain wherein 1-2 methylene units of the chain are independently and optionally replaced with —C(O)—, —C(S)—, —CR₂—, —CF2-, —CRF—, —CR(OR)—, —S—, —S(O)—, or —S(O)₂, and wherein L^{X} is optionally substituted with halogen or —R^{o}.

In some embodiments, L^{X} is an optionally substituted C₁₋₅ bivalent straight or branched saturated or unsaturated hydrocarbon chain wherein one methylene unit is replaced with —C(O)—. In some embodiments, L^{X} is an optionally substituted C₁₋₄ bivalent straight or branched saturated or unsaturated hydrocarbon chain wherein one methylene unit is replaced with —C(O)—. In some embodiments, L^{x} is an optionally substituted C₁₋₃ bivalent straight or branched saturated or unsaturated hydrocarbon chain wherein one methylene unit is replaced with —C(O)—. In some embodiments, L^{X} is an optionally substituted C₃ bivalent straight or branched saturated or unsaturated hydrocarbon chain wherein one methylene unit is replaced with —C(O)—. In some embodiments, L^{X} is an optionally substituted C₃ bivalent straight saturated or unsaturated hydrocarbon chain wherein one methylene unit is replaced with —C(O)—.

In some embodiments, L^{X} is a C₁₋₅ bivalent straight or branched saturated or unsaturated hydrocarbon chain wherein one methylene unit is replaced with —C(O)—, and wherein L^{X} is optionally substituted with halogen or —R^{o}. In some embodiments, L^{X} is a C₁-4 bivalent straight or branched saturated or unsaturated hydrocarbon chain wherein one methylene unit is replaced with —C(O)—, and wherein L^{X} is optionally substituted with halogen or —R^{o}. In some embodiments, L^{X} is a C₁₋₃ bivalent straight or branched saturated or unsaturated hydrocarbon chain wherein one methylene unit is replaced with —C(O)—, and wherein L^{x} is optionally substituted with halogen or —R^{o}. In some embodiments, L^{X} is a C₃ bivalent straight or branched saturated or unsaturated hydrocarbon chain wherein one methylene unit is replaced with —C(O)—, and wherein L^{X} is optionally substituted with halogen or —R^{o}. In some embodiments, L^{X} is a C₃ bivalent straight saturated or unsaturated hydrocarbon chain wherein one methylene unit is replaced with —C(O)—, and wherein L^{X} is optionally substituted with halogen or —R^{o}.

In some embodiments, L^{x} is —C(F)—CH-(optionally substituted C₁₋₃ bivalent straight or branched saturated or unsaturated hydrocarbon chain)-. In some embodiments, L^{x} is —C(CF₃)—N(R^{†})-(optionally substituted C₁₋₃ bivalent straight or branched saturated or unsaturated hydrocarbon chain)-

In some embodiments, L^{X} is a covalent bond or #-L^{XA}-L^{XB}-, wherein:
# represents the point of attachment to Ring X:
L^{XA} is -Cy^{x}-, —C(O)—, —C(S)—, —CR₂—, —CR(OR)—, —C(NR)—, —S(O)—, —S(O)₂— —S(O)(NR)—; and
L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

As described above and defined herein. L^{XA} is -Cy^{x}-, —C(O)—, —C(S)—, —CR₂—, —CR(OR)—, —C(NR)—, —S(O)—, —S(O)₂— or —S(O)(NR)—. In some embodiments, L^{XA} is -Cy^{x}-. In some embodiments, L^{XA} is —C(O)—, —C(S)—, —CR₂—, —CR(OR)—, —C(NR)—, —S(O)—, —S(O)₂— or —S(O)(NR)—.

In some embodiments, L^{XA} is —C(O)— or —C(S)—. In some embodiments, L^{XA} is —C(O)—, —S(O)—, —S(O)₂— or —S(O)(NR)—. In some embodiments, L^{XA} is —S(O)—, —S(O)₂— or —S(O)(NR)—. In some embodiments, L^{XA} is —C(O)—. In some embodiments, L^{XA} is —C(S)—. In some embodiments, L^{XA} is —CR₂—. In some embodiments, L^{XA} is —CR₂—, wherein each R of L^{XA} is independently hydrogen or optionally substituted C₁₋₆ aliphatic. In some embodiments, L^{XA} is —CR₂—, wherein each R of L^{XA} is independently hydrogen or C₁₋₆ aliphatic substituted with halogen (e.g., fluoro). In some embodiments, L^{XA} is —CR(OR)—. In some embodiments, L^{XA} is —C(NR)—. In some embodiments, L^{XA} is —S(O)—. In some embodiments, L^{XA} is —S(O)₂—. In some embodiments, L^{XA} is —S(O)(NR)—.

In some embodiments, L^{XA} is an optionally substituted phenyl. In some embodiments, L^{XA} is an optionally substituted 3-7 membered saturated or partially unsaturated carbocyclylenyl. In some embodiments, L^{XA} is an optionally substituted 3-7 membered saturated or partially unsaturated heterocyclylenyl having 1-4 heteroatoms independently selected from nitrogen, oxygen, and sulfur. In some embodiments, L^{XA} is an optionally substituted 5-9 membered monocyclic or bicyclic heteroarylenyl having 1-4 heteroatoms independently selected from nitrogen, oxygen, and sulfur, some embodiments, L^{XA} is an optionally substituted 5-6 membered monocyclic heteroarylenyl having 1-4 heteroatoms independently selected from nitrogen, oxygen, and sulfur. In some embodiments, L^{XA} is an optionally substituted 5 membered monocyclic heteroarylenyl having 1-4 heteroatoms independently selected from nitrogen, oxygen, and sulfur. In some embodiments, L^{XA} is an optionally substituted 6 membered monocyclic heteroarylenyl having 1-4 heteroatoms independently selected from nitrogen, oxygen, and sulfur.

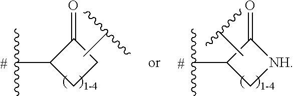

In some embodiments, L^{XA} is an optionally substituted 1-4 or 1-4 In some embodiments, L^{XA} is an optionally substituted

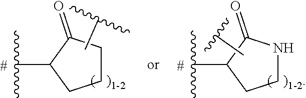

In some embodiments, L^{XA} is an optionally substituted:

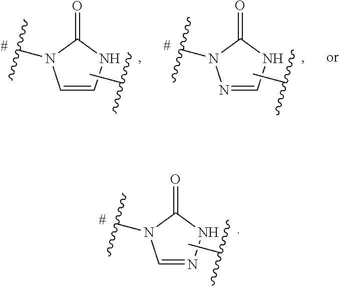

In some embodiments, L^{XA} is:

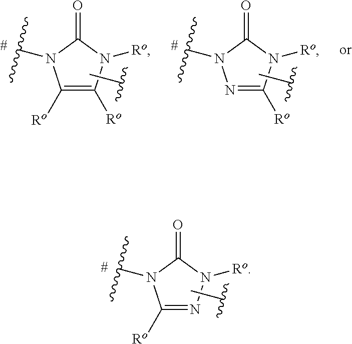

In some such embodiments, R^{o} is hydrogen or C₁₋₆ aliphatic.

In some embodiments, L^{XA} is an optionally substituted:

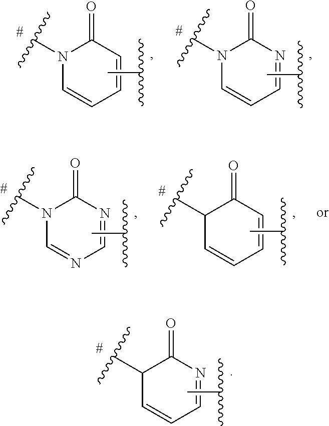

In some embodiments, L^{XA} is:

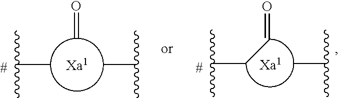

wherein Ring Xa¹ is an optionally substituted 3-7 membered saturated or partially unsaturated carbocyclic or heterocyclic ring having 1-2 heteroatoms selected from nitrogen, oxygen, and sulfur, or a 5-6 membered monocyclic heteroaryl ring having 1-4 heteroatoms independently selected from nitrogen, oxygen, and sulfur.

As defined above and described herein, Ring Xa¹ is an optionally substituted 3-7 membered saturated or partially unsaturated carbocyclyl or heterocyclyl having 1-2 heteroatoms selected from nitrogen, oxygen, and sulfur, or a 5-6 membered monocyclic heteroaryl having 1-4 heteroatoms independently selected from nitrogen, oxygen, and sulfur. In some embodiments, Ring Xa¹ is an optionally substituted 3-7 membered saturated or partially unsaturated carbocyclyl. In some embodiments, Ring Xa¹ is an optionally substituted 3-7 membered saturated or partially unsaturated heterocyclyl having 1-2 heteroatoms selected from nitrogen, oxygen, and sulfur, 5-6 membered monocyclic heteroaryl having 1-4 heteroatoms independently selected from nitrogen, oxygen, and sulfur.

In some embodiments, Ring Xa¹ an optionally substituted 5 membered monocyclic heteroaryl ring having 1-4 heteroatoms independently selected from nitrogen, oxygen, and sulfur. In some embodiments, Ring Xa¹ is an optionally substituted 6 membered monocyclic heteroaryl ring having 1-4 heteroatoms independently selected from nitrogen, oxygen, and sulfur.

In some embodiments, L^{XA} is

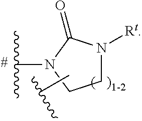

In some embodiments, L^{XA} is optionally substituted

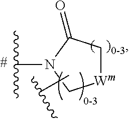

where W^{m} is as described above and defined herein. In some embodiments, L^{XA} is

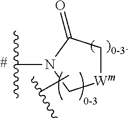

In some embodiments, L^{XA} is optionally substituted

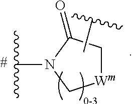

In some embodiments, L^{XA} is

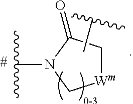

In some embodiments, L^{XA} is

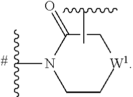

In some embodiments, L^{XA} is

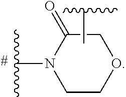

In some embodiments, L^{XA} is an optionally substituted ring selected from:

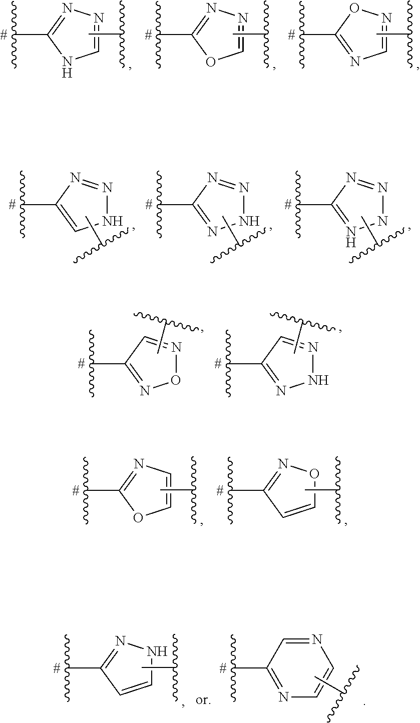

In some embodiments, L^{XA} is:

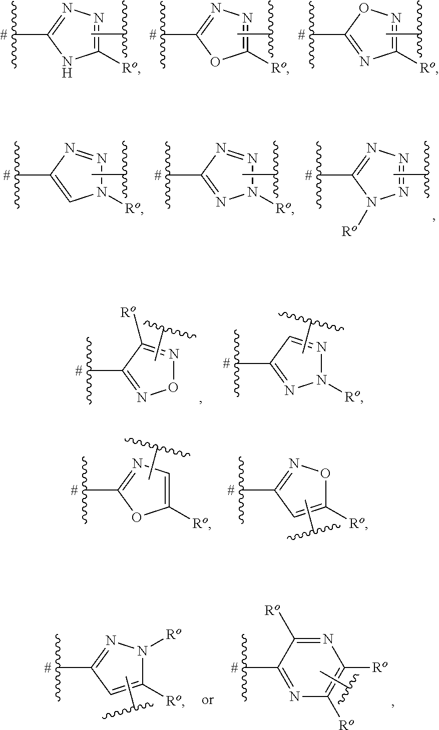

wherein each R^{o} is a defined above and described herein (e.g., hydrogen or C₁₋₆ aliphatic.

In some embodiments, L^{XA} is:

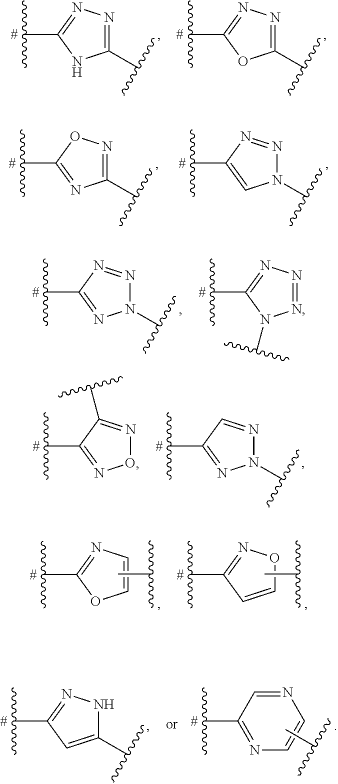

As described above and defined herein, L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L^{XB} is a covalent bond. In some embodiments, L^{XB} is an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{XB} is an optionally substituted C₄ bivalent straight or branched saturated or unsaturated hydrocarbon chain. In some embodiments, L^{XB} is an optionally substituted C₁₋₄ bivalent straight or branched saturated or unsaturated hydrocarbon chain, wherein 1 methylene unit of the chain is optionally replaced with —O—, —NR—, or —S—.

In some embodiments, L^{XB} is an optionally substituted C₁₋₃ bivalent straight or branched saturated or unsaturated hydrocarbon chain. In some embodiments, L^{XB} is an optionally substituted C₁₋₃ bivalent straight or branched saturated or unsaturated hydrocarbon chain, wherein 1 methylene unit of the chain is optionally replaced with —O—, —NR—, or —S—. In some embodiments, L^{XB} is —NR-(optionally substituted C₁₋₄ bivalent straight or branched saturated or unsaturated hydrocarbon chain)-. In some embodiments, L^{XB} is —O-(optionally substituted C₁₋₄ bivalent straight or branched saturated or unsaturated hydrocarbon chain)-. In some embodiments, L^{XB} is —S-(optionally substituted C₁₋₄ bivalent straight or branched saturated or unsaturated hydrocarbon chain)-.

In some embodiments, L^{XB} is optionally substituted —CH₂—, —CH₂CH₂—, —CH₂CH₂CH₂—,

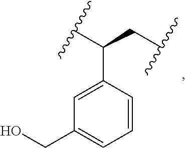 [SMILES: *C[C@H](*)c1cccc(CO)c1]

—CH₂CH₂CH₂O—, —CH₂C(O)O—, —CH₂CH₂C(O)O—, —NR—, or —NRCH₂CH₂—. In some embodiments, L^{XB} is —CH₂—, —CH₂CH₂—, —CH₂CH₂CH₂—, —CH₂CH₂CH₂O—, —CH₂C(O)O—, —CH₂CH₂C(O)O—,

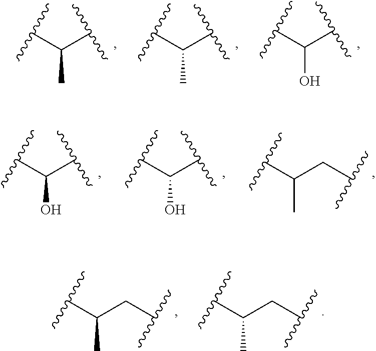

As described above and defined herein, each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur.

In some embodiments, Cy^{x}- is an optionally substituted ring selected from phenyl, a 3-6 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur.

In some embodiments, -Cy^{x}- is an optionally substituted phenyl. In some embodiments, -Cy^{x}- is an optionally substituted 3-6 membered saturated or partially unsaturated carbocyclylenyl. In some embodiments, -Cy^{x}- is an optionally substituted 3-6 membered saturated or partially unsaturated heterocyclylenyl having 1-4 heteroatoms independently selected from nitrogen, oxygen, and sulfur. In some embodiments, -Cy^{x}- is an optionally substituted 5-9 membered monocyclic or bicyclic heteroarylenyl having 1-4 heteroatoms independently selected from nitrogen, oxygen, and sulfur.

In some embodiments -Cy^{x}- is

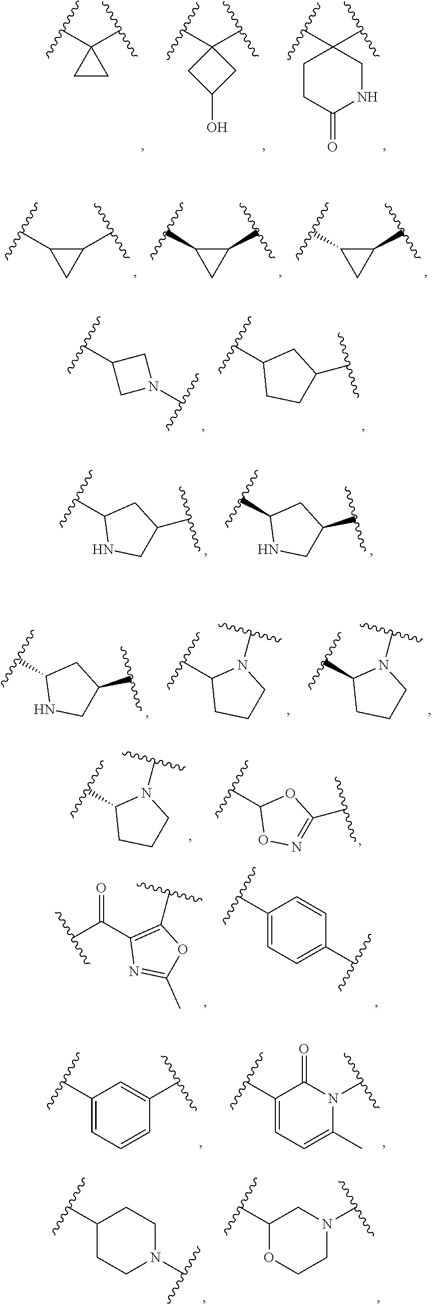

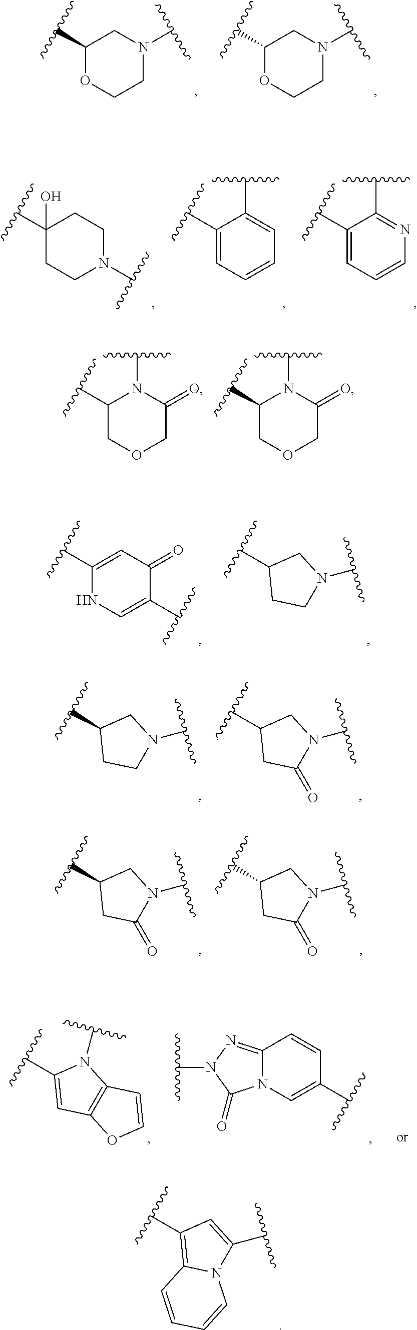

In some embodiment, -Cy^{x}- is as depicted in the compounds of Table 1A, below.

In some embodiments, L^{X} is —C(O)(CR₂)₁₋₃—. In some embodiments, L^{X} is —C(O)(CH₂)₁₋₃—.

In some embodiments, L^{x} is —C(O)—, —C(O)CH₂—, —S(O)₂CH₂—, —C(O)CH₂CH₂—, —C(O)OCH₂—, —C(O)CH₂O—, —C(O)CH₂CH₂CH₂—, —C(O)CH₂CH₂S(O)₂—, —C(O)CH₂CH₂CO₂—, —C(O)CH₂NHC(O)—, C(O)CH₂N(Me)S(O)₂—,

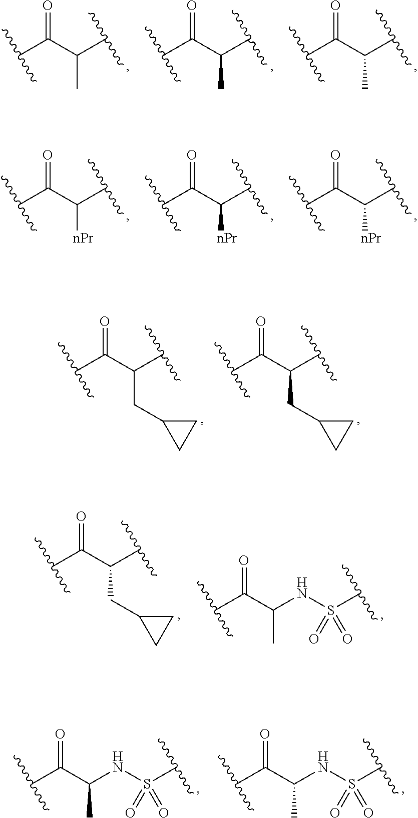

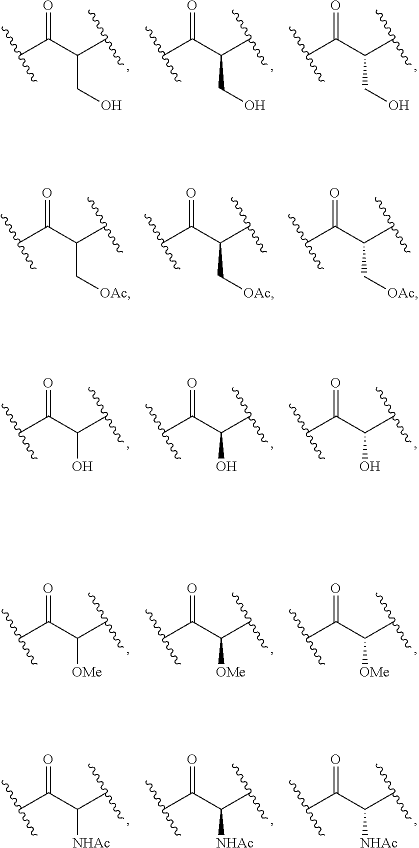

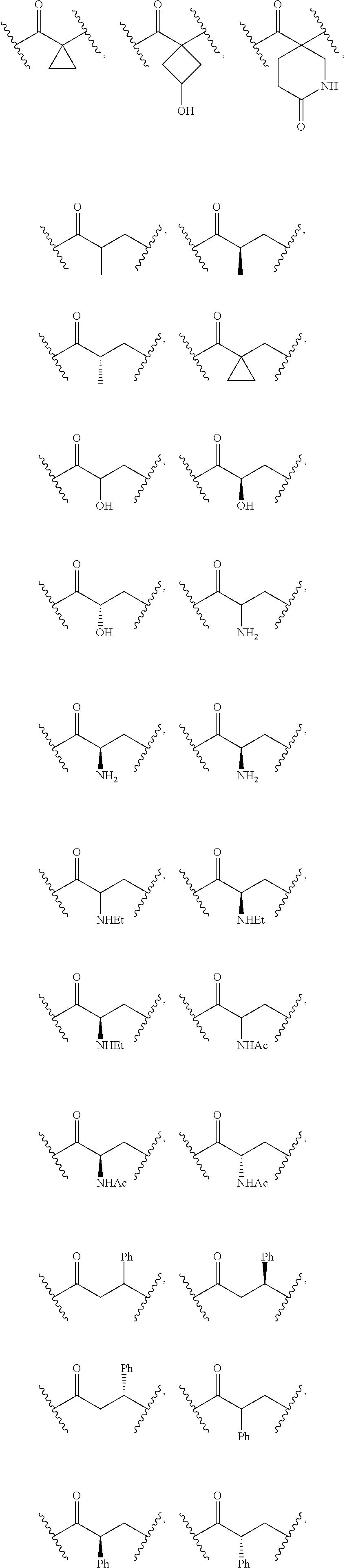

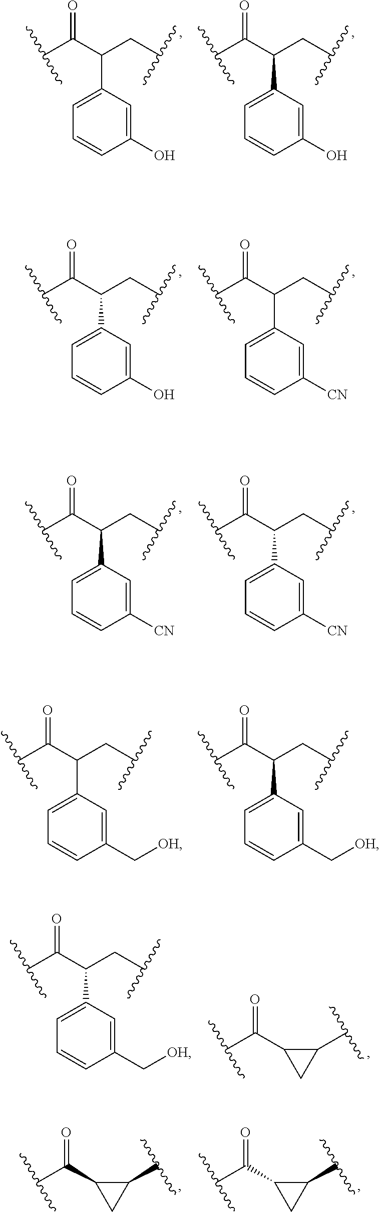

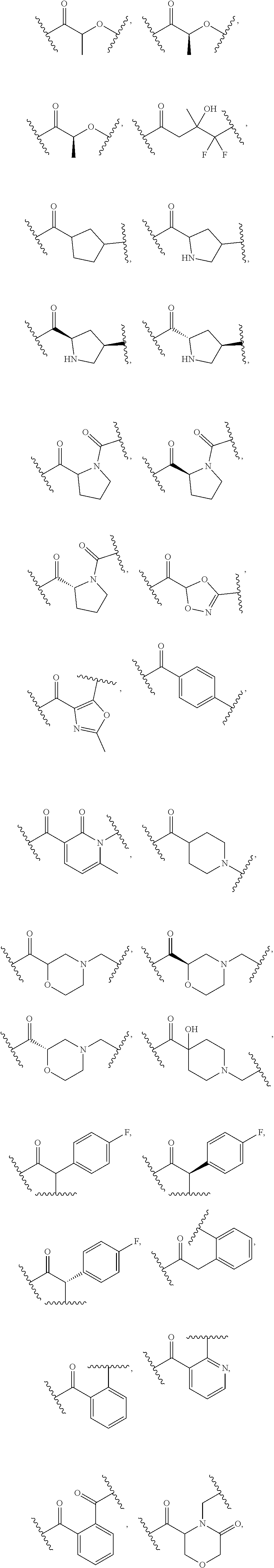

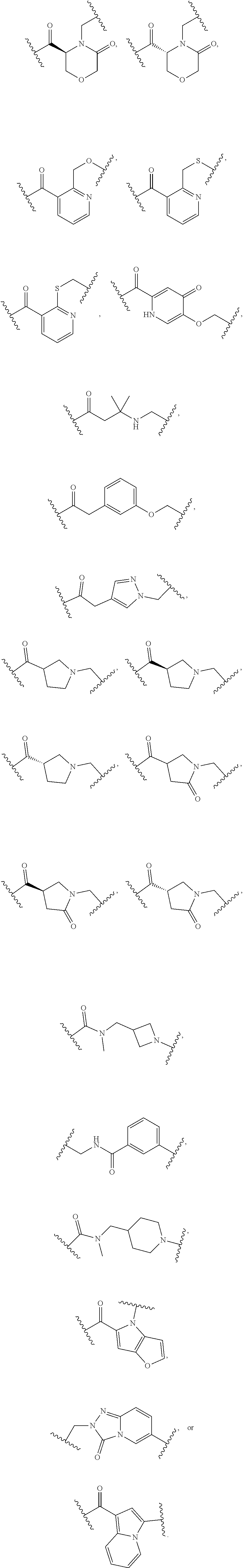

In some embodiments, L^{x} is

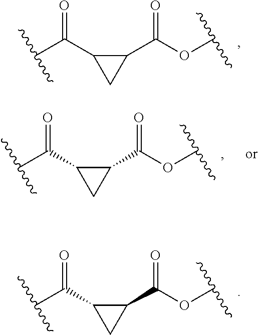

In some embodiments, L^{x} is —C(O)CH₂CH₂—.

In some embodiments, L^{x} is —S(O)₂CH₂CH₂—.

In some embodiment, L^{x} is as depicted in the compounds of Table 1A, below.

In some embodiments, -Ring X-L^{x}- is:

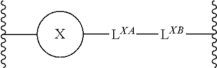

or a pharmaceutically acceptable salt thereof, wherein each of Ring X, L^{XA}, and L^{XB} is as defined above and described herein.

In some embodiments, -Ring X-L^{x}- is:

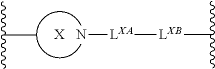

or a pharmaceutically acceptable salt thereof, wherein each of Ring X, L^{XA}, and L^{XB} is as defined above and described herein.

In some embodiments, -Ring X-L^{x}- is:

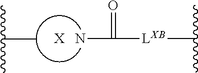

or a pharmaceutically acceptable salt thereof, wherein each of Ring X and L^{XB} is as defined above and described herein.

As described above and defined herein, each of w, x, and y are independently 0, 1, 2, 3, or 4.

In some embodiments, w is 0. In some embodiments, w is 1. In some embodiments, w is 2. In some embodiments, w is 3. In some embodiments, w is 4. In some embodiments, w is 0 or 1. In some embodiments, w is 1 or 2.

In some embodiments, x is 0. In some embodiments, x is 1. In some embodiments, x is 2. In some embodiments, x is 3. In some embodiments, x is 4. In some embodiments, x is 0 or 1. In some embodiments, x is 1 or 2.

In some embodiments, y is 0. In some embodiments, y is 1. In some embodiments, y is 2. In some embodiments, y is 3. In some embodiments, y is 4. In some embodiments, y is 0 or 1. In some embodiments, y is 1 or 2.

In some embodiment, w, x, and y are as depicted in the compounds of Table 1A, below.

In some embodiments, SBM is as depicted in the compounds of Table 1A, below.

In certain embodiments, the present invention provides methods of degrading STAT6, or a mutant thereof, or methods of treating a STAT6-mediated disease, disorder, or condition, comprising administering compound of formula I as a compound of any one of the following formulae:

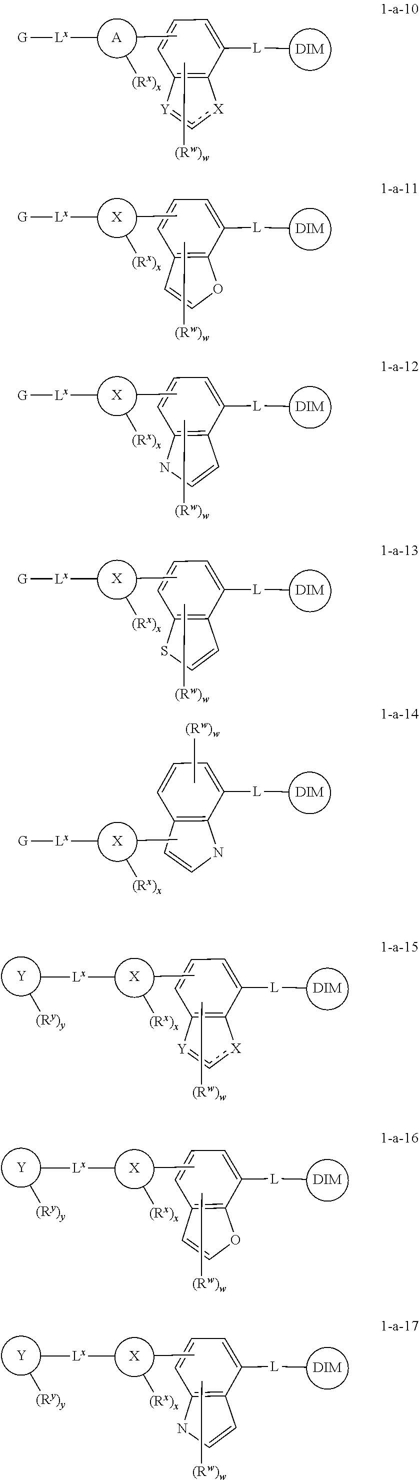

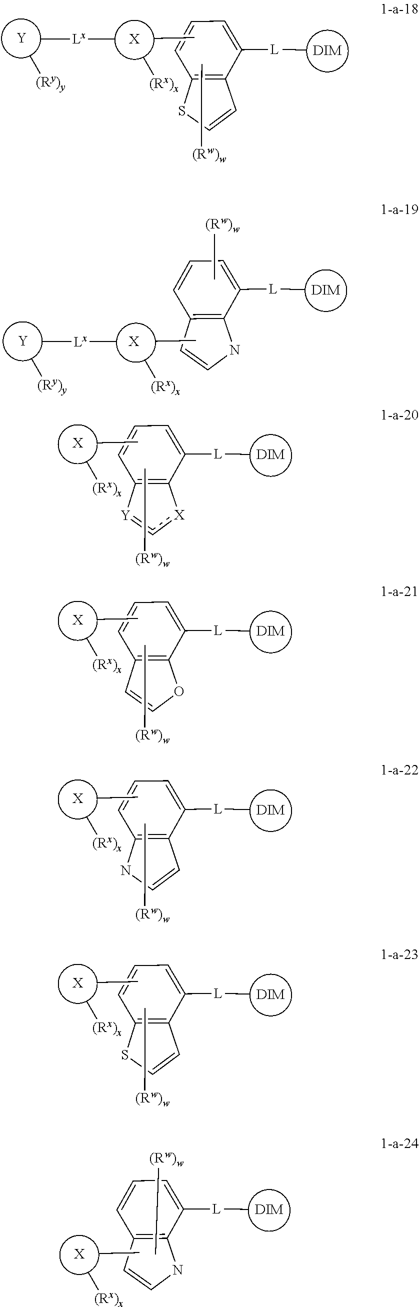

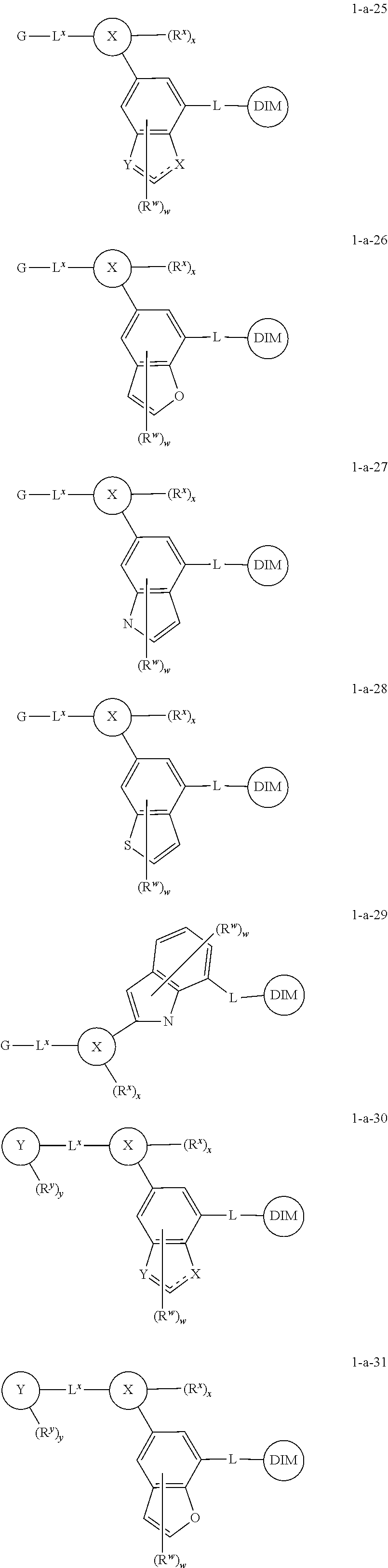

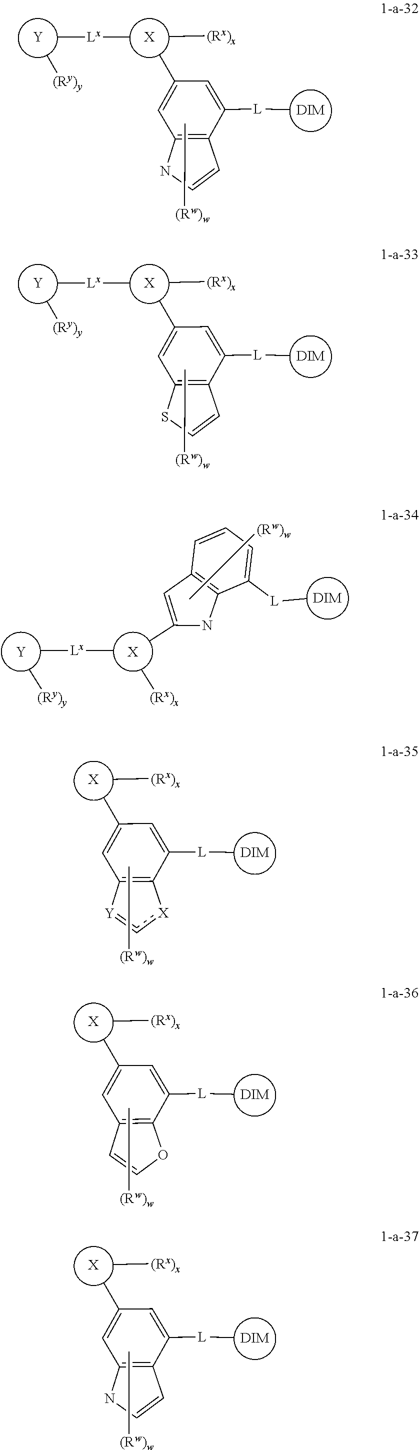

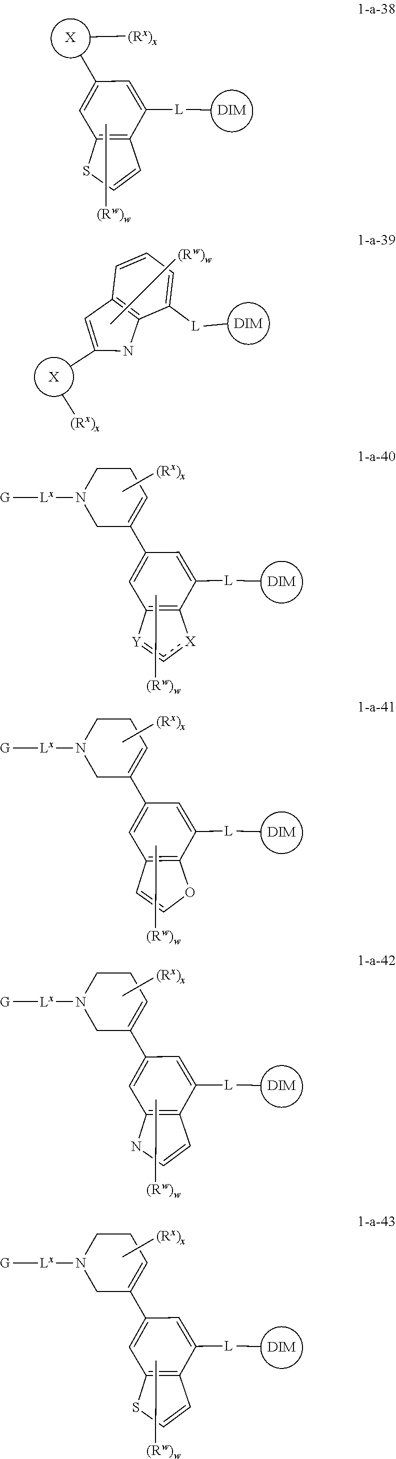

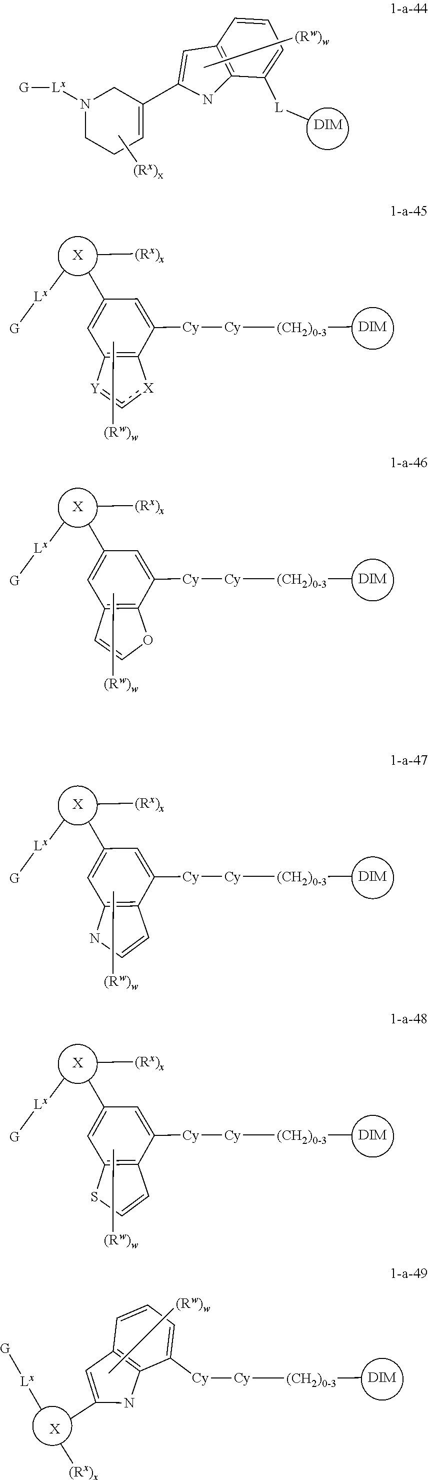

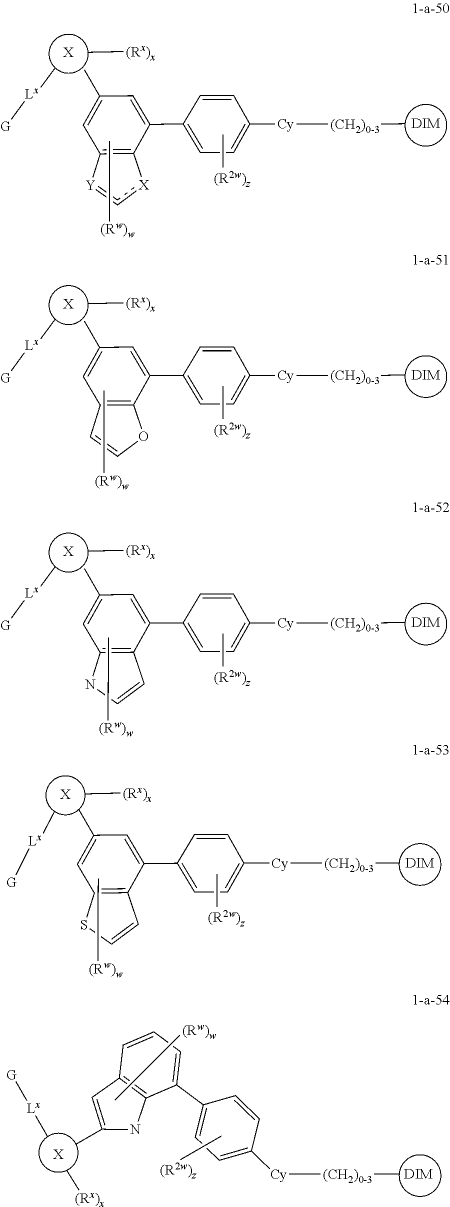

or a pharmaceutically acceptable salt thereof, wherein:
X is CH and Y is N—R^{w}, X is CH and Y is S, or X is O and Y is CH;
each R^{2w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R; and
z is 0, 1, 2, 3, or 4;
wherein DIM, L, -Cy-, Ring X. Ring Y, R, R^{w}, R^{x}, R^{y}, L^{x}, w, x, and y are as defined above and described herein both individually and in combination.

In some embodiments, X is CH and Y is N—R^{w}, X is CH and Y is S, or X is O and Y is CH.

In some embodiments, X is CH and Y is N—R^{w}. In some embodiments, X is CH and Y is NH. In some embodiments, X is CH and Y is S. In some embodiments, X is O and Y is CH.

In some embodiments, X and Y are as depicted in the compounds of Table 1A, below.

As described above and defined herein, R^{2w} is selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{2w} is hydrogen. In some embodiments, R^{2w} is R^{A}. In some embodiments, R^{2w} is halogen. In some embodiments, R^{2w} is —CN. In some embodiments, R^{2w} is —NO₂. In some embodiments, R^{2w} is —OR. In some embodiments, R^{2w} is —SR. In some embodiments, R^{2w} is —NR₂. In some embodiments, R^{2w} is —SiR₃. In some embodiments, R^{2w} is —S(O)₂R. In some embodiments, R^{2w} is —S(O)₂NR₂. In some embodiments, R^{2w} is —S(O)(NR)R. In some embodiments, R^{2w} is —S(O)R. In some embodiments, R^{2w} is —C(O)R. In some embodiments, R^{2w} is —C(O)OR. In some embodiments, R^{2w} is —C(O)NR₂. In some embodiments, R^{2w} is —C(O)NROR. In some embodiments, R^{2w} is —OC(O)R. In some embodiments, R^{2w} is —OC(O)NR₂. In some embodiments, R^{2w} is —P(O)R₂. In some embodiments, R^{2w} is —P(O)(OR)₂. In some embodiments, R^{2w} is —OP(O)R₂. In some embodiments, R^{2w} is —OP(O)(OR)₂. In some embodiments, R^{2w} is —OP(O)(OR)NR₂. In some embodiments, R^{2w} is —OP(O)(NR₂)₂. In some embodiments, R^{2w} is —NRC(O)OR. In some embodiments, R^{2w} is —NRC(O)R. In some embodiments, R^{2w} is —NRC(O)N(R)₂. In some embodiments, R^{2w} is —NP(O)R₂. In some embodiments, R^{2w} is —NRP(O)(OR)₂. In some embodiments, R^{2w} is —NRP(O)(OR)NR₂. In some embodiments, R^{2w} is —NRP(O)(NR₂)₂. In some embodiments, R^{2w} is —NRS(O)₂R.

In some embodiments, each R^{2w} is independently hydrogen, C₁ 6 alkyl, C₁₋₆ haloalkyl, halogen, —OC₁₋₆ alkyl, or —OC₁₋₆ haloalkyl.

In some embodiments, each R^{2w} is independently fluoro, chloro, methyl, ethyl, —CHF₂, —CMeF₂, —CF₃, —OMe, —OEt, —OCHF₂, —OCMeF₂, or —OCF₃.

In some embodiments, R^{2w} is fluoro. In some embodiments, R^{2w} is chloro. In some embodiments, R^{2w} is fluoro and chloro. In some embodiments, R^{2w} is —OMe.

In some embodiments, R^{2w} is as depicted in the compounds of Table 1A, below.

As described above and defined herein, z is 0, 1, 2, 3, or 4.

In some embodiments, z is 0. In some embodiments, z is 1. In some embodiments, z is 2. In some embodiments, z is 3. In some embodiments, z is 4. In some embodiments, z is 0 or 1. In some embodiments, z is 1 or 2.

In some embodiment, z is as depicted in the compounds of Table 1A, below.

In certain embodiments, the present invention provides methods of degrading STAT6, or a mutant thereof, or methods of treating a STAT6-mediated disease, disorder, or condition, comprising administering compound of formula I-a″″-1 as any one of the following formulae:

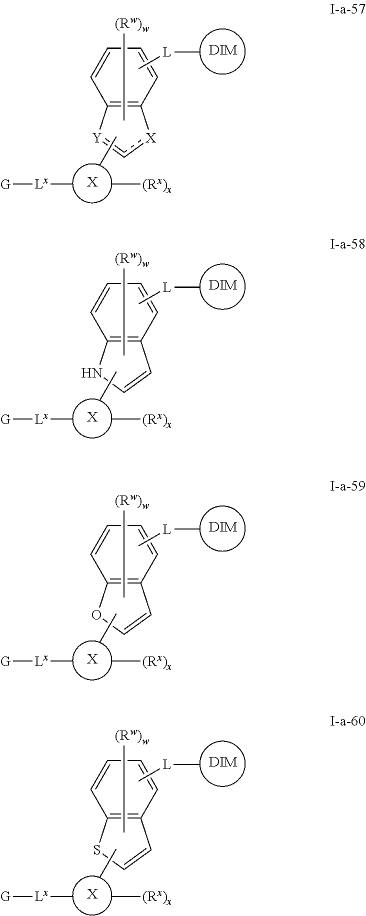

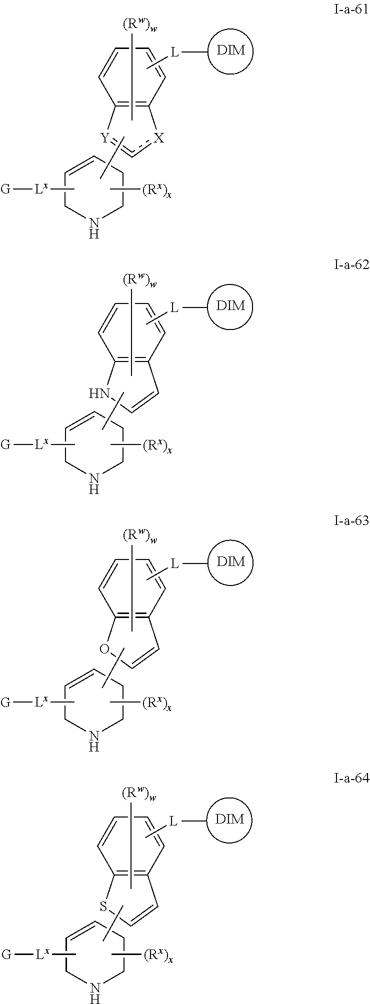

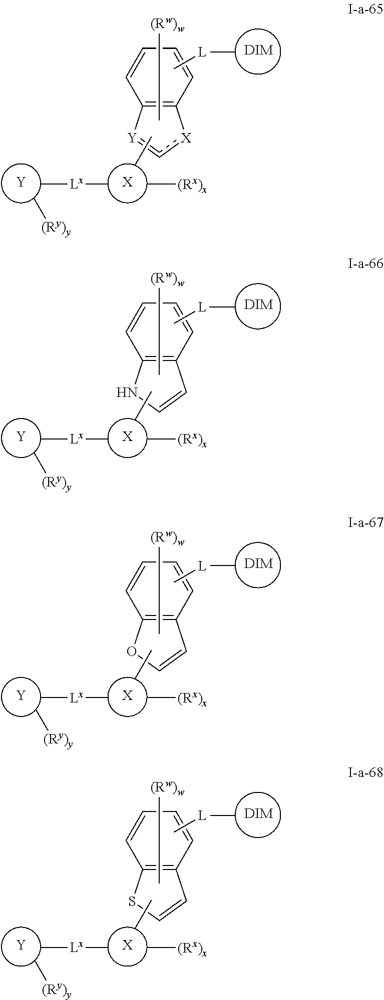

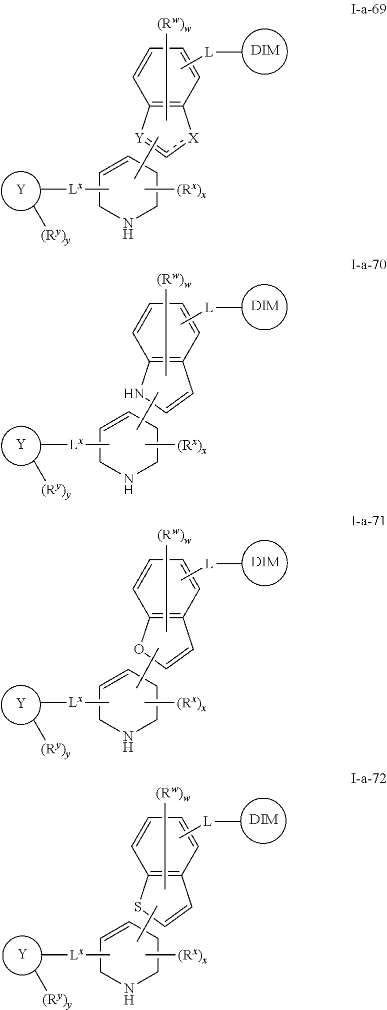

or a pharmaceutically acceptable salt thereof, wherein each of the variables are as defined above and described herein both individually and in combination, and wherein:
each of X and Y of Ring W is independently N, NH, N—R^{W}, —O—, —S—, C—H, C—R^{W}, C—H₂, CH(R^{W}), or C—(R^{W})₂.

### Ligase Binding Moiety (LBM)

In some embodiments, DIM is LBM. In some embodiments, LBM is an E3 ligase ligand. In some embodiments, LBM comprises means for binding an E3 ubiquitin ligase. In some embodiments, LBM comprises means for binding a cereblon E3 ubiquitin ligase.

As defined herein and described below, wherein a formula is depicted using square brackets,

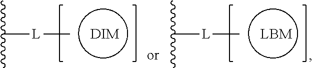

L is attached to a modifiable carbon, oxygen, or nitrogen atom within DIM or LBM including substitution or replacement of a defined group in DIM or LBM.

In certain embodiments, the present invention provides methods of degrading STAT6, or a mutant thereof, or methods of treating a STAT6-mediated disease, disorder, or condition, comprising administering compound of formula I, wherein LBM is a compound of formula I-aa:

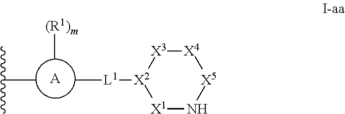

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

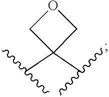 [SMILES: *C1(*)COC1]

X² is N, C—R^{B}, Si—R^{B}, or P═O;
X³ and X⁴ are independently a covalent bond, —CR₂—, —CF₂—, —O—, —S—, or X³—X⁴ is —CR═CR—;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂, —NP(O)R₂, —N(R)P(O)(OR)₂, —N(R)P(O)(OR)(NR₂), —N(R)P(O)(NR₂)₂, —N(R)S(O)₂R;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur;
m is 0, 1, 2, 3, 4, or 5.

In certain embodiments, the present invention provides methods of degrading STAT6, or a mutant thereof, or methods of treating a STAT6-mediated disease, disorder, or condition, comprising administering compound of formula I, wherein LBM is of formula I-aa′:

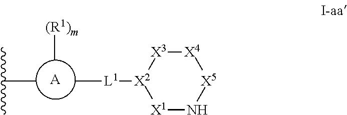

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

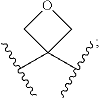 [SMILES: *C1(*)COC1]

X² is N, C—R^{B}, Si—R^{B}, or P═O;
X³ and X⁴ are independently a covalent bond, —CR₂—, —CF₂—, —O—, —S—, or X³—X⁴ is —CR═CR—;
each R¹ is independently —H. R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In certain embodiments, the present invention provides methods of degrading STAT6, or a mutant thereof, or methods of treating a STAT6-mediated disease, disorder, or condition, comprising administering compound of formula I, wherein LBM is a compound of formula I-aa-1:

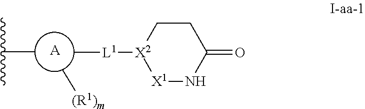

or a pharmaceutically acceptable salt thereof, wherein:
X¹ is a bivalent moiety selected from —CH₂— or —C(O)—;
X² is N or CH;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is a ring selected from phenylenyl, naphthalenyl, pyridinylenyl,

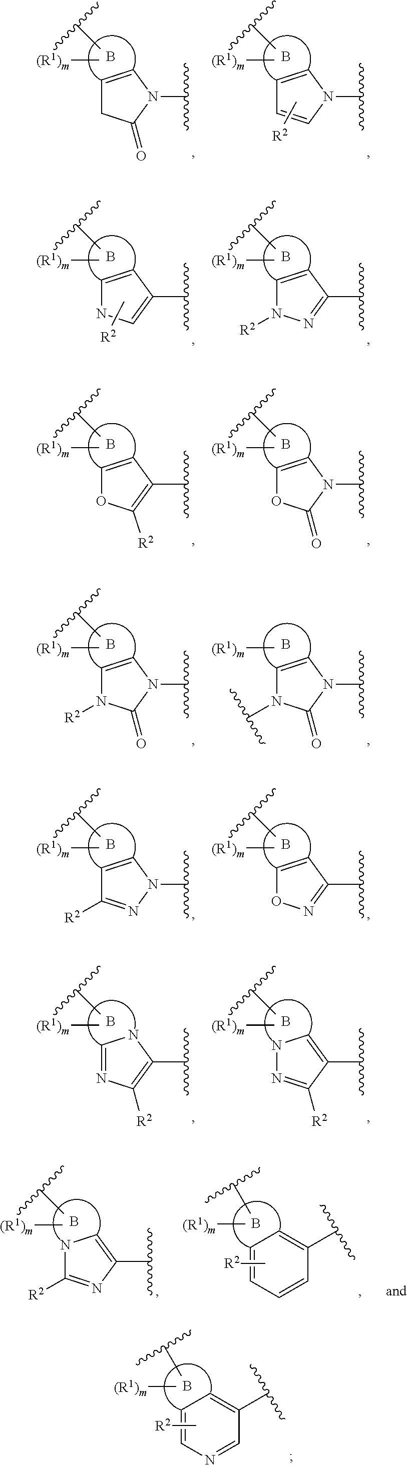

Ring B is a fused ring selected from benzo or a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R;
R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

In certain embodiments, the present invention provides methods of degrading STAT6, or a mutant thereof, or methods of treating a STAT6-mediated disease, disorder, or condition, comprising administering a compound of formula I, wherein LBM is of formula I-aa-1′:

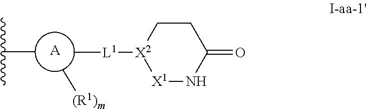

or a pharmaceutically acceptable salt thereof, wherein:
X is a bivalent moiety selected from —CH₂— or —C(O)—;
X¹ is N or CH;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is a ring selected from phenylenyl, naphthalenyl, pyridinylenyl,

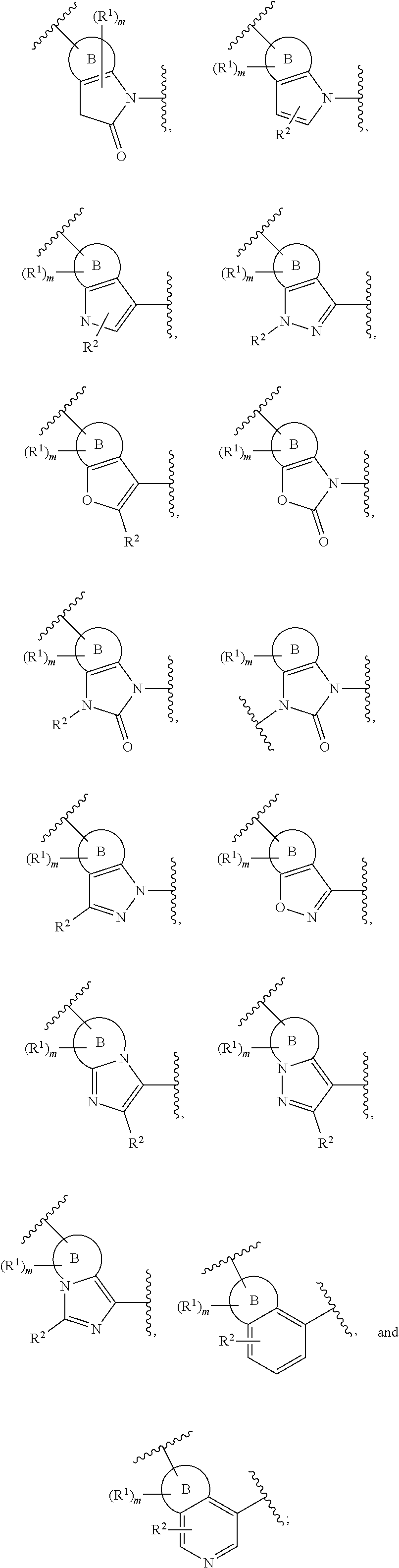

Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR—N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 3-10 membered heteroaryl having 1-4 heteroatoms independently selected from nitrogen, oxygen, or sulfur;
R² is hydrogen, halogen, C₁₋₆ alkyl, C₃₋₆cycloalkyl, C₁₋₆ haloalkyl, —OC₃₋₆ alkyl, —OC₃₋₆ cycloalkyl, or —OC₁₋₆ haloalkyl; or:
   an R² and an R¹ are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo: or a 5-10 membered heteroaryl having 1-4 heteroatoms independently selected from nitrogen, oxygen, or sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

For formula I-aa-1 and I-aa-1′, it will be appreciated that an occurrence of R² reduces the available occurrences of m by 1.

In some embodiments, the present invention provides methods of degrading STAT6, or a mutant thereof, or methods of treating a STAT6-mediated disease, disorder, or condition, comprising administering a compound of formula I-aa-1 as a compound of any one of the following formulae:

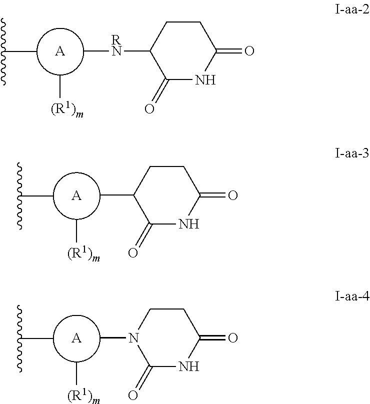

or a pharmaceutically acceptable salt thereof.

In some embodiments, the present invention provides methods of degrading STAT6, or a mutant thereof, or methods of treating a STAT6-mediated disease, disorder, or condition, comprising administering a compound of formula I-aa′ or I-aa-1′ as a compound of any one of the following formulae:

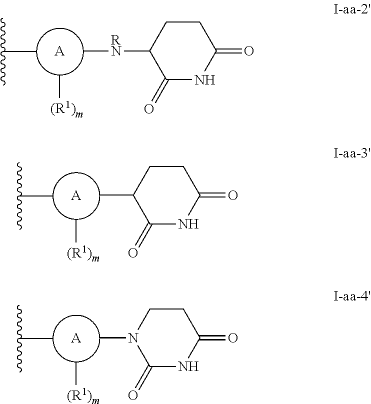

or a pharmaceutically acceptable salt thereof, wherein each R¹, m, Ring A, and R is as defined above in I-aa′ or I-aa-1′ and described herein both individually and in combination.

In some embodiments, the present invention provides methods of degrading STAT6, or a mutant thereof, or methods of treating a STAT6-mediated disease, disorder, or condition, comprising administering a compound of formula I-aa′ or I-aa-1′ as a compound of any one of the following formulae:

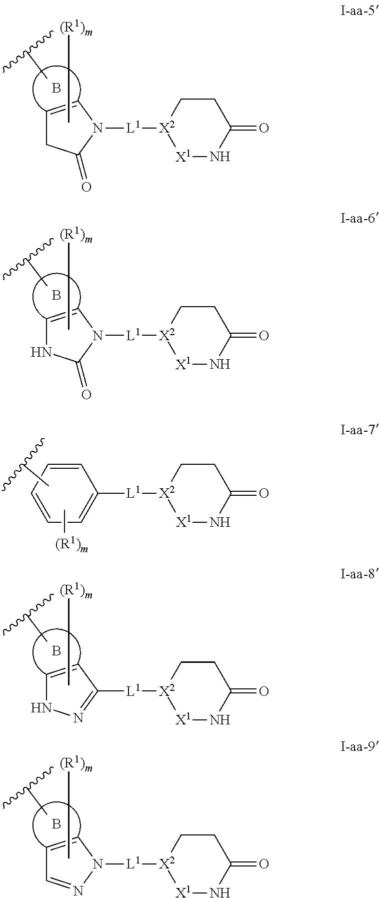

or a pharmaceutically acceptable salt thereof, wherein each L¹, R¹, m, X¹, X², and Ring B is as defined above in I-aa′ or I-aa-1′ and described herein both individually and in combination.

In some embodiments, the present invention provides methods of degrading STAT6, or a mutant thereof, or methods of treating a STAT6-mediated disease, disorder, or condition, comprising administering a compound of formula I-aa′ or I-aa-1′ as a compound of any one of the following formulae:

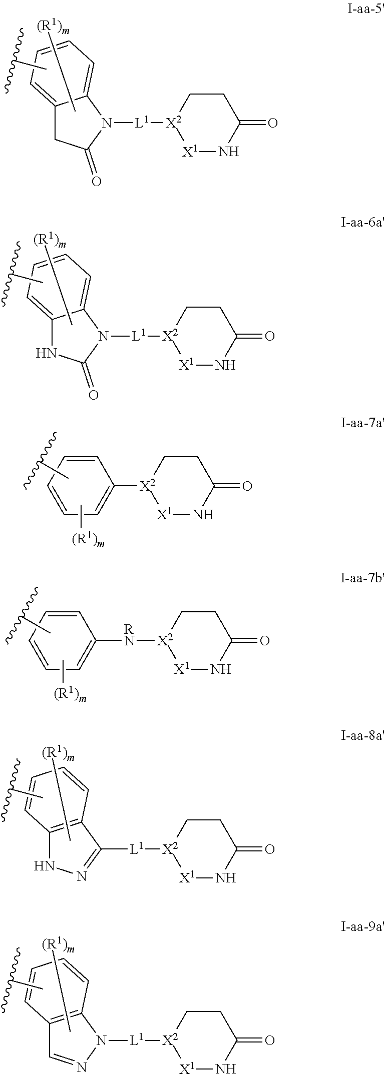

or a pharmaceutically acceptable salt thereof, wherein each L, R¹, m, X¹, and X² is as defined above in I-aa′ or I-aa-1′ and described herein both individually and in combination.

In some embodiments, the present invention provides methods of degrading STAT6, or a mutant thereof, or methods of treating a STAT6-mediated disease, disorder, or condition, comprising administering a compound of formula I-aa′ or I-aa-1′ as a compound of any one of the following formulae:

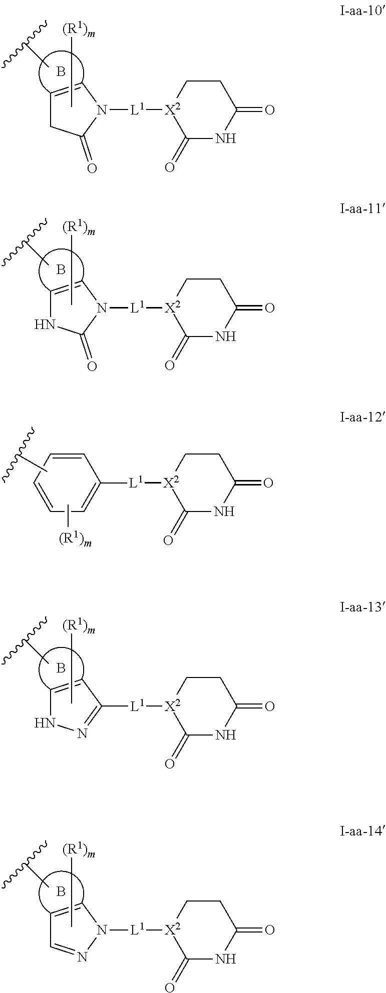

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition, comprising administering a compound of formula I-aa′ or I-aa-1′ as a compound of any one of the following formulae:

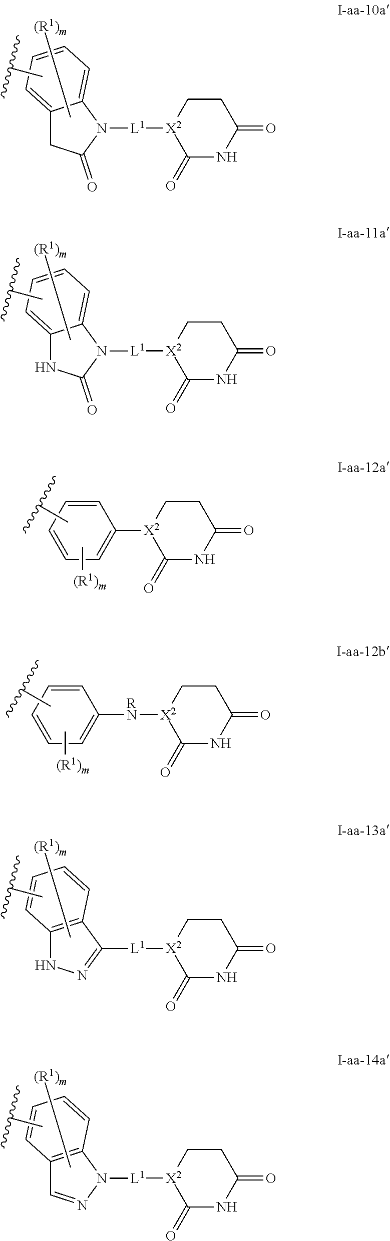

or a pharmaceutically acceptable salt thereof, wherein each variable is as defined above and described herein both individually and in combination.

As defined above and described herein, X¹ and X⁵ are independently a covalent bond, —CR₂—, —SO₂—, —S(O)—, —P(O)R—, —P(O)OR—, —P(O)N(R)₂—, —C(O)—, —C(S)—, or

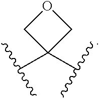 [SMILES: *C1(*)COC1]

In some embodiments, X¹ is a covalent bond. In some embodiments, X¹ is —CR₂—. In some embodiments, X¹ is —SO₂—. In some embodiments, X¹ is —S(O)—. In some embodiments, X¹ is —P(O)R—. In some embodiments, X¹ is —P(O)OR—. In some embodiments, X¹ is —P(O)N(R)₂—. In some embodiments, X¹ is —C(O)—. In some embodiments, X¹ is —C(S)—, or

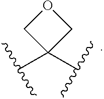 [SMILES: *C1(*)COC1]

In some embodiments, X¹ is —CH₂—. In some embodiments, X¹ is —C(O)—.

In some embodiments, X¹ is selected from those depicted in the compounds of Table 1A below.

In some embodiments, X⁵ is a covalent bond. In some embodiments, X⁵ is —CR₂—. In some embodiments, X is —SO₂—. In some embodiments, X⁵ is —S(O)—. In some embodiments, X⁵ is —P(O)R—. In some embodiments, X⁵ is —P(O)OR—. In some embodiments, X⁵ is —P(O)N(R)₂—. In some embodiments, X⁵ is —C(O)—. In some embodiments, X is —C(S)—, or

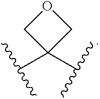 [SMILES: *C1(*)COC1]

In some embodiments, X is —CH₂—. In some embodiments, X⁵ is —C(O)—.

In some embodiments, X⁵ is selected from those depicted in the compounds of Table 1A below.

As defined above and described herein, X² is N, C—R^{B}, Si—R^{B}, or P═O. In some embodiments, X² is N. In some embodiments, X² is C—R^{B}. In some embodiments, X² is Si—R^{B}. In some embodiments, X² is P═O. In some embodiments, X² is CH.

In some embodiments, X² is selected from those depicted in the compounds of Table 1A below.

As defined above and described herein, X and X⁴ are independently a covalent bond, —CR₂—, —CF₂—, —O—, —S—, or X³-X⁴ is —CR═CR—. In some embodiments, X³ is —CR₂—. In some embodiments, X³ is —CF₂—. In some embodiments, X³ is

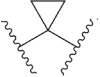 [SMILES: *C1(*)CC1]

In some embodiments, X³ is —O—. In some embodiments, X³ is —S—.

In some embodiments, K is selected from those depicted in the compounds of Table 1A below.

In some embodiments, X⁴ is —CR₂—. In some embodiments, X⁴ is —CF₂—. In some embodiments, X⁴ is

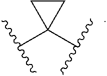 [SMILES: *C1(*)CC1]

In some embodiments, X⁴ is —O—. In some embodiments, X⁴ is —S—.

In some embodiments, X³—X⁴ is —CR═CR—.

In some embodiments, X⁴ is selected from those depicted in the compounds of Table 1A below.

As defined above and described herein, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R: or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R.

In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo: or a 5-10 membered heteroaryl having 1-4 heteroatoms independently selected from nitrogen, oxygen, or sulfur.

In some embodiments, R¹ is R^{A}. In some embodiments, R¹ is —CN. In some embodiments, R¹ is —NO₂. In some embodiments, R¹ is —OR. In some embodiments, one or more of R¹ is —Si(OH)₂R. In some embodiments, R¹ is —Si(OH)R₂. In some embodiments, R¹ is —SR. In some embodiments, R¹ is —NR₂. In some embodiments, R¹ is —SiR. In some embodiments, R¹ is —S(O)₂R. In some embodiments, R¹ is —S(O)₂NR₂. In some embodiments, R¹ is —S(O)R. In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —CR₂N(R)C(O)R. In some embodiments, R¹ is —CR₂N(R)C(O)NR₂. In some embodiments, R¹ is —CFR₂. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —OC(O)R. In some embodiments, R¹ is —OC(O)NR₂. In some embodiments, R¹ is —OP(O)R₂. In some embodiments, R¹ is —OP(O)(OR)₂. In some embodiments, R¹ is —OP(O)(OR)NR₂. In some embodiments, R¹ is independently —OP(O)(NR₂)₂—. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)P(O)R₂. In some embodiments, R¹ is —N(R)P(O)(OR)₂. In some embodiments, R¹ is —N(R)P(O)(OR)NR₂. In some embodiments, R¹ is —N(R)P(O)(NR₂)₂. In some embodiments, R¹ is —N(R)S(O)₂R.

In some embodiments, R¹ is halogen, C₁ 6alkyl, —OC₁ 6alkyl, C₁₋₆ haloalkyl, —OC₁₋₆ alkyl, or —OC₁₋₆ haloalkyl.

In some embodiments, R¹ is hydrogen. In some embodiments, R¹ is fluoro. In some embodiments, R¹ is chloro. In some embodiments, R¹ is methyl. In some embodiments, R¹ is —C(OH)Me₂. In some embodiments, R¹ is —CHF₂. In some embodiments, R¹ is —CF₃. In some embodiments, R¹ is —OMe.

In some embodiments, two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R¹ groups of Ring A are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, two R¹ groups of Ring A are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R¹ groups of Ring A are taken together with their intervening atoms to form an optionally substituted benzo.

In some embodiments, two R¹ groups of Ring A are taken together with their intervening atoms to form an optionally substituted ring selected from a 5-10 membered heteroaryl having 1-4 heteroatoms independently selected from nitrogen, oxygen, or sulfur. In some embodiments, two R¹ groups of Ring A are taken together with their intervening atoms to form an optionally substituted ring selected from a 5-6 membered heteroaryl having 1-4 heteroatoms independently selected from nitrogen, oxygen, or sulfur.

In some embodiments, two R¹ groups on the same atom of Ring A are taken together to form an optionally substituted ring selected from a 3-10 membered saturated or partially unsaturated carbocyclyl or heterocyclyl having 1-4 heteroatoms independently selected from nitrogen, oxygen, or sulfur; benzo. In some embodiments, two R¹ groups on the same atom of Ring A are taken together to form an optionally substituted ring selected from a 3-10 membered saturated or partially unsaturated carbocyclyl. In some embodiments, two R¹ groups on the same atom of Ring A are taken together to form an optionally substituted ring selected from a 3-6 membered saturated or partially unsaturated carbocyclyl. In some embodiments, two R¹ groups on the same atom of Ring A are taken together to form an optionally substituted ring selected from a 3 membered saturated or partially unsaturated carbocyclyl. In some embodiments, two R¹ groups on the same atom of Ring A are taken together to form an optionally substituted ring selected from a 3-10 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3-6 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted cyclopropyl ring.

In some embodiments, R¹ is selected from those depicted in the compounds of Table 1A below.

As defined above and described herein, R is hydrogen, halogen, C₁₋₆ alkyl, C₃₋₆ cycloalkyl, C₁₋₆ haloalkyl, —OC₁₋₆ alkyl, —OC₃₋₆ cycloalkyl, or —OC₁₋₆ haloalkyl; or: two R² groups are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, R² is hydrogen, halogen, C₁₋₆ alkyl. C₃₋₆ cycloalkyl, C₁₋₆ haloalkyl, —OC₁₋₆ alkyl, —OC₃₋₆ cycloalkyl, or —OC₁₋₆ haloalkyl.

In some embodiments, R² is hydrogen. In some embodiments, R² is halogen. In some embodiments, R² is C₁₋₆ alkyl. In some embodiments, R² is C₃₋₆ cycloalkyl. In some embodiments, R² is C₁₋₆ haloalkyl. In some embodiments, R² is —OC₁₋₆ alkyl. In some embodiments, R² is —OC₃₋₆ cycloalkyl. In some embodiments, R² is —OC₁₋₆ haloalkyl.

In some embodiments, R² is methyl. In some embodiments, R² is ethyl. In some embodiments, R² is cyclopropyl.

In some embodiments, an R² and an R¹ group are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, an R² and an R¹ group are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, an R² and an R¹ group are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, an R² and an R¹ group are taken together with their intervening atoms to form an optionally substituted benzo.

In some embodiments, an R² and an R¹ group are taken together with their intervening atoms to form an optionally substituted ring selected from a 5-10 membered heteroaryl having 1-4 heteroatoms independently selected from nitrogen, oxygen, or sulfur. In some embodiments, an R² and an R¹ group are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in the compounds of Table 1A below.

As defined above and described herein, each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R^{A} is an optionally substituted C₁₋₆ aliphatic. In some embodiments, each R^{A} is an optionally substituted phenyl. In some embodiments, each R^{A} is an optionally substituted 3-10 membered saturated or partially unsaturated carbocyclic ring. In some embodiments, each R^{A} is an optionally substituted 3-10 membered heterocyclic ring having 1-2 heteroatoms independently selected from nitrogen, oxygen, and sulfur. In some embodiments, each R^{A} is an optionally substituted 5-6 membered heteroaryl ring having 1-4 heteroatoms independently selected from nitrogen, oxygen, and sulfur.

As defined above and described herein, each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic.

In some embodiments, R^{B} is hydrogen. In some embodiments, R^{B} is halogen. In some embodiments, R^{B} is —CN. In some embodiments, R^{B} is —OR. In some embodiments, R^{B} is —SR. In some embodiments, R^{B} is —S(O)R. In some embodiments, R^{B} is —S(O)₂R. In some embodiments, R^{B} is —NR₂In some embodiments, R^{B} is —P(O)(OR)₂. In some embodiments, R^{B} is —P(O)(NR₂)OR. In some embodiments, R^{B} is —P(O)(NR₂)₂. In some embodiments, R^{B} is —Si(OH)₂R. In some embodiments, R is —Si(OH)R₂. In some embodiments, R^{B} is —SiR₃. In some embodiments, R^{B} is an optionally substituted C₁₋₄ aliphatic.

In some embodiments, R^{B} is C₁₋₄ aliphatic optionally substituted with 1-3 halogens. In some embodiments, R^{B} is C₁₋₄ aliphatic. In some embodiments, R^{B} is methyl. In some embodiments, R^{B} is fluoro.

In some embodiments, R^{B} is selected from those depicted in the compounds of Table 1A below.

As defined above and described herein. L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L¹ is a covalent bond. In some embodiments, L¹ is a Cia bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L¹ is a covalent bond, —O—, —NR—, —S—, —CR₂—, —NRC(O)—, or —C(O)NR—. In some embodiments, L¹ is —O—, —NR—, —S—, —CR—, —NRC(O)—, or —C(O)NR—. In some embodiments, L¹ is -0-. In some embodiments, L¹ is —NR—. In some embodiments, L¹ is —S—. In some embodiments, L¹ is —CR₂—. In some embodiments, L¹ is —CH₂—. In some embodiments, L¹ is —NRC(O)—. In some embodiments, L¹ is —C(O)NR—. In some embodiments, L¹ is —NHC(O)—. In some embodiments, L¹ is —C(O)NH—.

In some embodiments, L¹ is selected from those depicted in the compounds of Table 1A below.

As defined above and described herein, Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur.

In some embodiments, Ring A is a phenylenyl. In some embodiments, Ring A is a naphthalenyl. In some embodiments, Ring A is pyridinylenyl. In some embodiments, Ring A is a 4-7 membered saturated or partially unsaturated carbocyclylenyl. In some embodiments, Ring A is a 4-7 membered saturated or partially unsaturated heterocyclylenyl having 1-3 heteroatoms independently selected from nitrogen, oxygen, and sulfur. In some embodiments, Ring A is an 8-10 membered bicyclic saturated or partially unsaturated heterocyclylenyl having 1-2 heteroatoms independently selected from nitrogen, oxygen, and sulfur. In some embodiments, Ring A is a 5-6 membered heteroarylenyl having 1-4 heteroatoms independently selected from nitrogen, oxygen, and sulfur. In some embodiments, Ring A is an 8-10 membered bicyclic heteroarylenyl having 1-5 heteroatoms independently selected from nitrogen, oxygen, and sulfur. In some embodiments, Ring A is an 8-15 membered saturated or partially unsaturated tricyclic heterocyclylenyl having 1-5 heteroatoms independently selected from nitrogen, oxygen, and sulfur. In some embodiments, Ring A is or an 8-15 membered tricyclic heteroarylenyl having 1-5 heteroatoms independently selected from nitrogen, oxygen, and sulfur.

In some embodiments, Ring A is a 9- or 10-membered saturated or partially unsaturated monocyclic or bicyclic heterocyclylenyl or heteroarylenyl containing 1-4 heteroatoms independently selected from nitrogen, oxygen, and sulfur.

In some embodiments, Ring A is an 8-10 membered saturated or partially unsaturated bicyclic heterocyclylenyl or heteroarylenyl containing 1-4 heteroatoms independently selected from nitrogen, oxygen, and sulfur. In some embodiments, Ring A is a 9- or 10-membered saturated or partially unsaturated bicyclic heterocyclylenyl or heteroarylenyl containing 1-4 heteroatoms independently selected from nitrogen, oxygen, and sulfur.

In some embodiments, Ring A is a 9-membered saturated or partially unsaturated bicyclic heterocyclylenyl or heteroarylenyl containing 1-4 heteroatoms independently selected from nitrogen, oxygen, and sulfur. In some embodiments, Ring A is a 9-membered saturated or partially unsaturated bicyclic heterocyclylenyl or heteroarylenyl containing 1 nitrogen and 1 oxygen heteroatom.

In some embodiments, Ring A is a 5,6-fused saturated or partially unsaturated bicyclic heterocyclylenyl or heteroarylenyl containing 1-4 heteroatoms independently selected from nitrogen, oxygen, and sulfur. In some embodiments, Ring A is a 5,6-fused saturated or partially unsaturated bicyclic heterocyclylenyl or heteroarylenyl containing 1 nitrogen and 1 oxygen heteroatom.

In some embodiments, Ring A is not phthalimide.

In some embodiments, Ring A is:

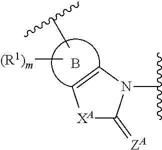

or a pharmaceutically acceptable salt thereof, wherein each R¹, Ring B, X^{A}, and m is as defined above and described herein both individually and in combination; and:
X^{A} is CH₂, CHR¹, C(R¹)₂, NH, NR¹, O or S.
Z^{A} is O, S, or NR.

As defined above and described herein, X^{A} is CH₂, CHR¹, C(R¹)₂, NH, NR¹, O or S. In some embodiments, X^{A} is CH₂. In some embodiments, X^{A} is CHR¹. In some embodiments, X^{A} is C(R¹)₂, NH, NR¹, O or S.

In some embodiments, X^{A} is C(R¹)₂, wherein each R¹ is optionally substituted C₁₋₆ aliphatic.

As defined above and described herein, Z^{A} is O S, or NR. In some embodiments, Z^{A} is O. In some embodiments, Z^{A} is S. In some embodiments, Z^{A} is NR.

In some embodiments, Ring A is:

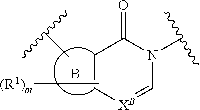

or a pharmaceutically acceptable salt thereof, wherein each R¹, Ring B, and m is as defined above and described herein both individually and in combination, and:
X^{B} is CR² or N.

As defined above and described herein, X^{B} is CR² or N. In some embodiments, X^{B} is CR². In some embodiments, X^{B} is N.

In some embodiments, Ring A is

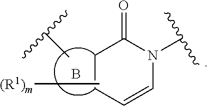

In some embodiments, Ring A is

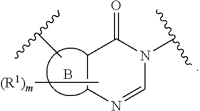

In some embodiments, Ring A is phenylenyl, naphthylenyl, pyridinylenyl,

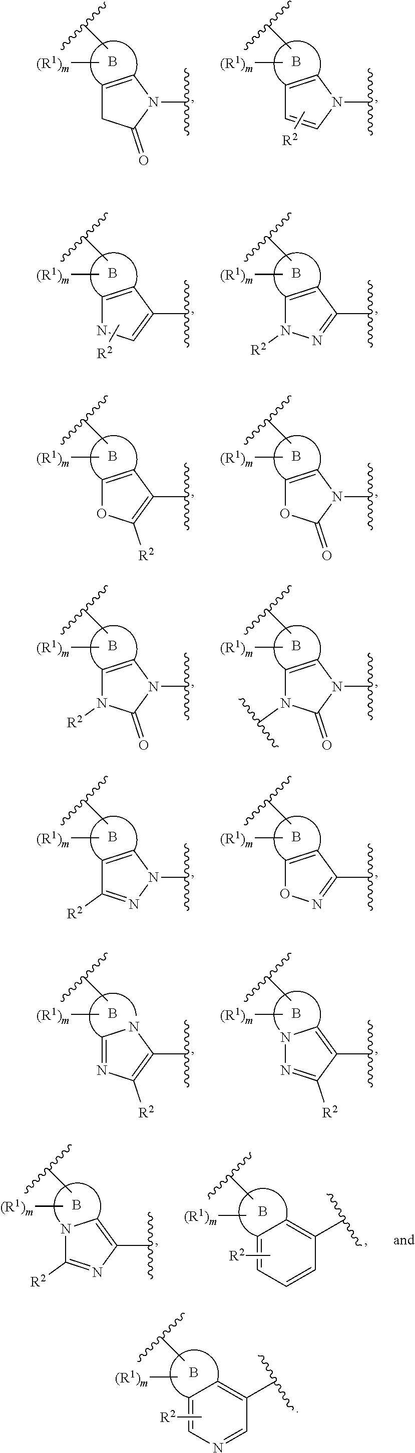

In some embodiments, Ring A is phenylenyl. In some embodiments, Ring A is naphthylenyl.

In some embodiments, Ring A is pyridinylenyl. In some embodiments, Ring A is

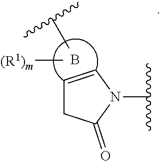

In some embodiments, Ring A is

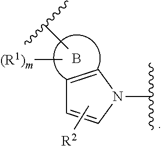

In some embodiments, Ring A is

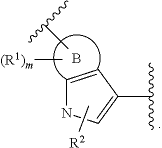

In some embodiments, Ring A is

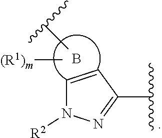

In some embodiments, Ring A is

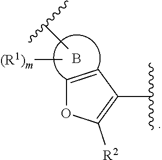

In some embodiments, Ring A is

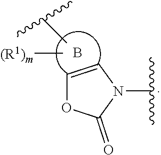

In some embodiments, Ring A is

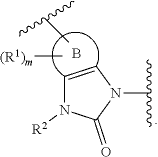

In some embodiments, Ring A is

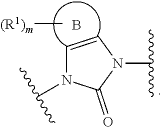

In some embodiments, Ring A is

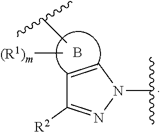

In some embodiments, Ring A is

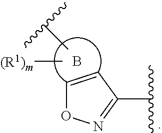

In some embodiments, Ring A is

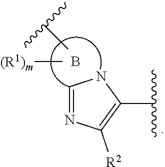

In some embodiments, Ring A is

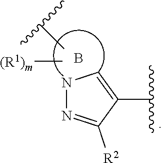

In some embodiments, Ring A is

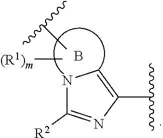

In some embodiments, Ring A is

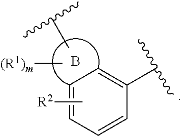

In some embodiments, Ring A is

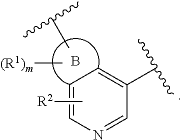

In some embodiments, Ring A is

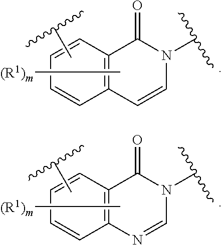

In some embodiments, Ring A is

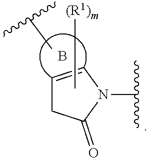

In some embodiments, Ring A is

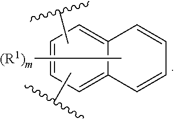

In some embodiments, Ring A is

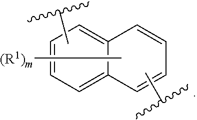

In some embodiments, Ring A is pyridinyl. In some embodiments, Ring A is

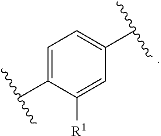

In some embodiments, Ring A is

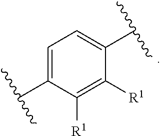

In some embodiments, Ring A is

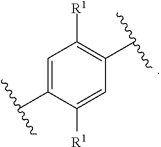

In some embodiments, Ring A is

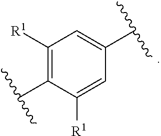

In some embodiments, Ring A is

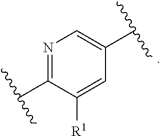

In some embodiments, Ring A is

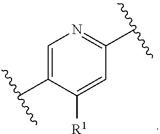

In some embodiments, Ring A is

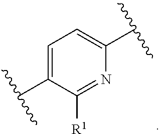

In some embodiments, Ring A is

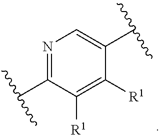

In some embodiments, Ring A is

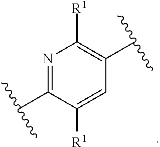

In some embodiments, Ring A is

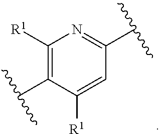

In some embodiments, Ring A is

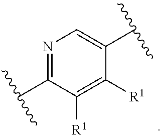

In some embodiments, Ring A is

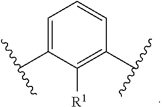

In some embodiments, Ring A is

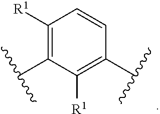

In some embodiments, Ring A is

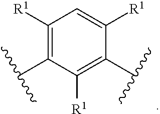

In some embodiments, Ring A is

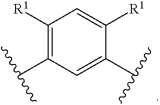

In some embodiments, Ring A is

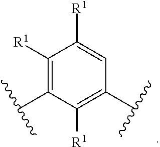

In some embodiments, Ring A is

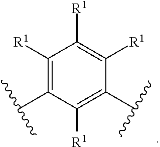

In some embodiments, Ring A is

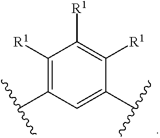

In some embodiments, Ring A is

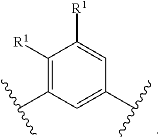

In some embodiments, Ring A is

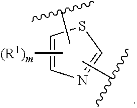

In some embodiments, Ring A is

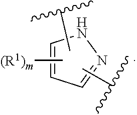

In some embodiments, Ring A is

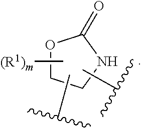

In some embodiments, Ring A is

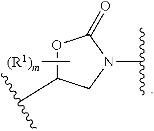

In some embodiments, Ring A is

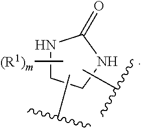

In some embodiments, Ring A is

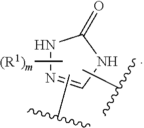

In some embodiments, Ring A is

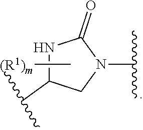

In some embodiments, Ring A is

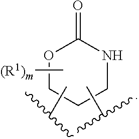

In some embodiments, Ring A is

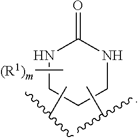

In some embodiments, Ring A is

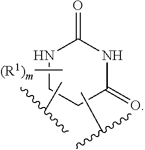

In some embodiments, Ring A is

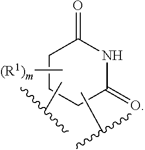

In some embodiments, Ring A is

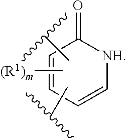

In some embodiments, Ring A is

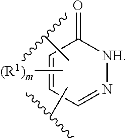

In some embodiments, Ring A is

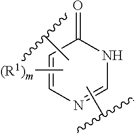

In some embodiments, Ring A is

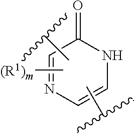

In some embodiments, Ring A is

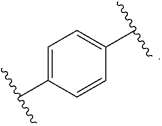 [SMILES: *c1ccc(*)cc1]

In other embodiments Ring A is a 6-10 membered monocyclic or bicyclic heteroaryl containing 1-4 heteroatoms independently selected from nitrogen, oxygen, and sulfur. In other embodiments Ring A is a 9-membered bicyclic heteroaryl containing 1-4 heteroatoms independently selected from nitrogen, oxygen, and sulfur. In some embodiments, Ring A is a 5,6-fused bicyclic heteroarylenyl containing 1-4 heteroatoms independently selected from nitrogen, oxygen, and sulfur.

In other embodiments Ring A is a 9-membered bicyclic heteroaryl containing 1 nitrogen and 1 oxygen heteroatom. In some embodiments, Ring A is a 5,6-fused bicyclic heteroarylenyl containing 1 nitrogen and 1 oxygen heteroatom. In some embodiments, Ring A is benzo[d]oxazolyl, benzo[d]thiazolyl, benzofuran, or benzo[b]thiophenyl. In some embodiments, Ring A is benzo[d]oxazolyl. In some embodiments, Ring A is benzo[d]thiazolyl. In some embodiments, Ring A is benzofuran. In some embodiments, Ring A is benzo[b]thiophenyl.

In other embodiments Ring A is a 9-membered bicyclic heteroaryl containing 1-3 nitrogen heteroatoms. In some embodiments, Ring A is a 5,6-fused bicyclic heteroarylenyl containing 1-3 nitrogen heteroatoms. In some embodiments, Ring A is indolyl, azaindolyl (e.g., 4-, 5-, 6-, or 7-azaindolyl), indazolyl, or azaindazolyl (e.g., 4-, 5-, 6-, or 7-azaindazolyl). In some embodiments, Ring A is indolyl. In some embodiments, Ring A is azaindolyl (e.g., 4-, 5-, 6-, or 7-azaindolyl). In some embodiments, Ring A is indazolyl. In some embodiments, Ring A is azaindazolyl (e.g., 4-, 5-, 6-, or 7-azaindazolyl).

In other embodiments Ring A is a 10-membered bicyclic heteroaryl containing 1-4 heteroatoms independently selected from nitrogen, oxygen, and sulfur. In some embodiments, Ring A is a 6,6-fused bicyclic heteroarylenyl containing 1-4 heteroatoms independently selected from nitrogen, oxygen, and sulfur. In some embodiments, Ring A is a 6,6-fused bicyclic heteroarylenyl containing 1 nitrogen heteroatom.

In some embodiments, Ring A is quinolinyl or isoquinolinyl. In some embodiments, Ring A is quinolinyl. In some embodiments, Ring A is isoquinolinyl.

In other embodiments Ring A is a 6-10 membered monocyclic or bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1 nitrogen heteroatom. In some embodiments, Ring A is a 5,6-fused bicyclic saturated or partially unsaturated heterocyclylenyl containing 1-4 heteroatoms independently selected from nitrogen, oxygen, and sulfur. In some embodiments, Ring A is a 5,6-fused bicyclic saturated or partially unsaturated heterocyclylenyl containing 1 nitrogen heteroatom.

In other embodiments Ring A is a 6-10 membered monocyclic or bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1 nitrogen heteroatom, and having an oxo group. In some embodiments, Ring A is a 5,6-fused bicyclic saturated or partially unsaturated heterocyclylenyl containing 1-4 heteroatoms independently selected from nitrogen, oxygen, and sulfur, and having an oxo group. In some embodiments, Ring A is a 5,6-fused bicyclic saturated or partially unsaturated heterocyclylenyl containing 1 nitrogen heteroatom, and having an oxo group.

In some embodiments, Ring A is indolinyl, indolinonyl, isoindolinyl, isoindolinonyl, isoindolinedionyl, pyrrolopyridinyl, or pyrrolopyrimidinyl. In some embodiments, Ring A is indolinyl. In some embodiments, Ring A is indolinonyl. In some embodiments, Ring A is isoindolinyl. In some embodiments, Ring A is isoindolinonyl. In some embodiments, Ring A is isoindolinedionyl. In some embodiments, Ring A is pyrrolopyridinyl. In some embodiments, Ring A is pyrrolopyrimidinyl.

In some embodiments, Ring A is benzoxazolyl, benzothiazolyl, indazolyl, or azaindazolyl. In some embodiments, Ring A is benzoxazolyl. In some embodiments, Ring A is benzothiazolyl. In some embodiments, Ring A is indazolyl. In some embodiments, Ring A is azaindazolyl (e.g., 4-, 5-, 6-, or 7-azaindazolyl).

In some embodiments, Ring A is 2,3-dihydrobenzofuranyl, indolinyl, or 2,3-dihydrobenzothiophenyl. In some embodiments, Ring A is 2,3-dihydrobenzofuranyl. In some embodiments, Ring A is indolinyl. In some embodiments, Ring A is 2,3-dihydrobenzothiophenyl.

In some embodiments, Ring A is

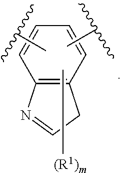

In some embodiments, Ring A is

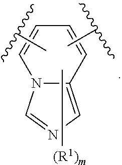

In some embodiments, Ring A is

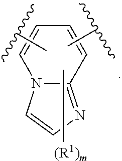

In some embodiments, Ring A is

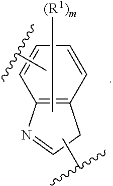

In some embodiments, Ring A is

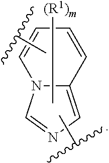

In some embodiments, Ring A is

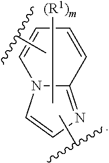

In some embodiments, Ring A is

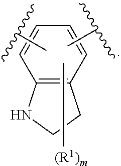

In some embodiments, Ring A is

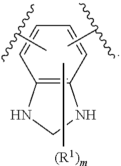

In some embodiments, Ring A is

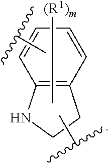

In some embodiments, Ring A is

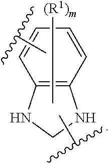

In some embodiments, Ring A is

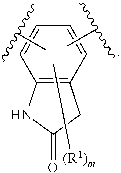

In some embodiments, Ring A is

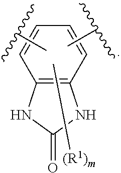

In some embodiments, Ring A is

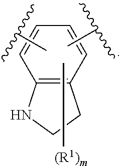

In some embodiments, Ring A is

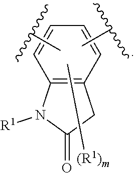

In some embodiments, Ring A is

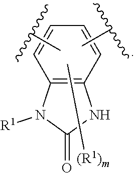

In some embodiments, Ring A is

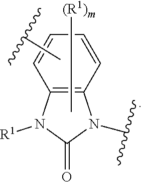

In some embodiments, Ring A is

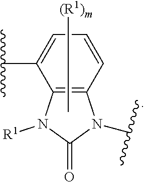

In some embodiments, Ring A is

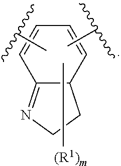

In some embodiments, Ring A is

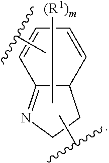

In some embodiments, Ring A is

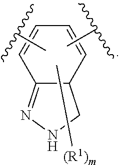

In some embodiments, Ring A is

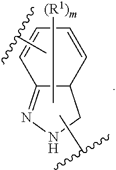

In some embodiments, Ring A is

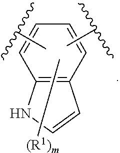

In some embodiments, Ring A is

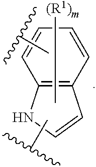

In some embodiments, Ring A is

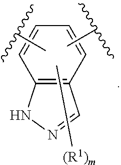

In some embodiments, Ring A is

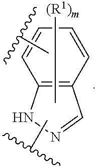

In some embodiments, Ring A is

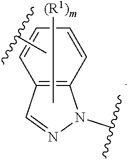

In some embodiments, Ring A is

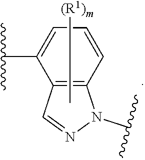

In some embodiments, Ring A is

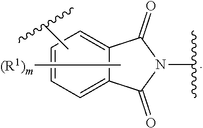

In some embodiments, Ring A is

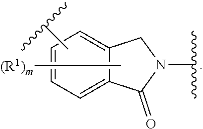

In some embodiments, Ring A is

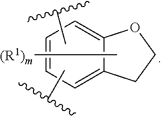

In some embodiments, Ring A is

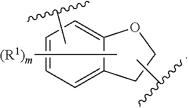

In some embodiments, Ring A is

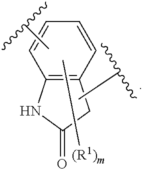

In some embodiments, Ring A is an 10-15 membered saturated or partially unsaturated tricyclic heterocyclylenyl having 1-5 heteroatoms independently selected from nitrogen, oxygen, and sulfur.

In some embodiments, Ring A is a 12-membered saturated or partially unsaturated tricyclic heterocyclylenyl having 1-5 heteroatoms independently selected from nitrogen, oxygen, and sulfur. In some embodiments, Ring A is a 12-membered saturated or partially unsaturated tricyclic heterocyclylenyl having 1-5 nitrogen heteroatoms. In some embodiments, Ring A is a 12-membered saturated or partially unsaturated tricyclic heterocyclylenyl having 3 nitrogen heteroatoms.

In some embodiments, Ring A is a 5,6,5-fused saturated or partially unsaturated tricyclic heterocyclylenyl having 1-5 heteroatoms independently selected from nitrogen, oxygen, and sulfur. In some embodiments, Ring A is a 5,6,5-fused membered saturated or partially unsaturated tricyclic heterocyclylenyl having 1-5 nitrogen heteroatoms. In some embodiments, Ring A is a 5,6,5-fused membered saturated or partially unsaturated tricyclic heterocyclylenyl having 3 nitrogen heteroatoms.

In some embodiments, Ring A is a 5,5,6-fused saturated or partially unsaturated tricyclic heterocyclylenyl having 1-5 heteroatoms independently selected from nitrogen, oxygen, and sulfur. In some embodiments, Ring A is a 5,5,6-fused membered saturated or partially unsaturated tricyclic heterocyclylenyl having 1-5 nitrogen heteroatoms. In some embodiments, Ring A is a 5,5,6-fused membered saturated or partially unsaturated tricyclic heterocyclylenyl having 3 nitrogen heteroatoms.

In some embodiments, Ring A is a 13-membered saturated or partially unsaturated tricyclic heterocyclylenyl having 1-5 heteroatoms independently selected from nitrogen, oxygen, and sulfur. In some embodiments, Ring A is a 13-membered saturated or partially unsaturated tricyclic heterocyclylenyl having 1-5 nitrogen heteroatoms. In some embodiments, Ring A is a 13-membered saturated or partially unsaturated tricyclic heterocyclylenyl having 3 nitrogen heteroatoms.

In some embodiments, Ring A is a 5,6,6-fused saturated or partially unsaturated tricyclic heterocyclylenyl having 1-5 heteroatoms independently selected from nitrogen, oxygen, and sulfur. In some embodiments, Ring A is a 5,6,6-fused membered saturated or partially unsaturated tricyclic heterocyclylenyl having 1-5 nitrogen heteroatoms. In some embodiments, Ring A is a 5,6,6-fused membered saturated or partially unsaturated tricyclic heterocyclylenyl having 3 nitrogen heteroatoms.

In some embodiments, Ring A is a 6,5,6-fused saturated or partially unsaturated tricyclic heterocyclylenyl having 1-5 heteroatoms independently selected from nitrogen, oxygen, and sulfur. In some embodiments, Ring A is a 6,5,6-fused membered saturated or partially unsaturated tricyclic heterocyclylenyl having 1-5 nitrogen heteroatoms. In some embodiments, Ring A is a 6,5,6-fused membered saturated or partially unsaturated tricyclic heterocyclylenyl having 3 nitrogen heteroatoms.

In some embodiments, Ring A is a 14-membered saturated or partially unsaturated tricyclic heterocyclylenyl having 1-5 heteroatoms independently selected from nitrogen, oxygen, and sulfur.

In some embodiments, Ring A is or an 10-15 membered tricyclic heteroarylenyl having 1-5 heteroatoms independently selected from nitrogen, oxygen, and sulfur.

In some embodiments, Ring A is a 12-membered saturated or partially unsaturated tricyclic heteroarylenyl having 1-5 heteroatoms independently selected from nitrogen, oxygen, and sulfur. In some embodiments, Ring A is a 12-membered saturated or partially unsaturated tricyclic heteroarylenyl having 1-5 nitrogen heteroatoms. In some embodiments, Ring A is a 12-membered saturated or partially unsaturated tricyclic heteroarylenyl having 3 nitrogen heteroatoms.

In some embodiments, Ring A is a 5,6,5-fused saturated or partially unsaturated tricyclic heteroarylenyl having 1-5 heteroatoms independently selected from nitrogen, oxygen, and sulfur. In some embodiments, Ring A is a 5,6,5-fused membered saturated or partially unsaturated tricyclic heteroarylenyl having 1-5 nitrogen heteroatoms. In some embodiments, Ring A is a 5,6,5-fused membered saturated or partially unsaturated tricyclic heteroarylenyl having 3 nitrogen heteroatoms.

In some embodiments, Ring A is a 5,5,6-fused saturated or partially unsaturated tricyclic heteroarylenyl having 1-5 heteroatoms independently selected from nitrogen, oxygen, and sulfur. In some embodiments, Ring A is a 5,5,6-fused membered saturated or partially unsaturated tricyclic heteroarylenyl having 1-5 nitrogen heteroatoms. In some embodiments, Ring A is a 5,5,6-fused membered saturated or partially unsaturated tricyclic heteroarylenyl having 3 nitrogen heteroatoms.

In some embodiments, Ring A is a 13-membered saturated or partially unsaturated tricyclic heteroarylenyl having 1-5 heteroatoms independently selected from nitrogen, oxygen, and sulfur. In some embodiments, Ring A is a 13-membered saturated or partially unsaturated tricyclic heteroarylenyl having 1-5 nitrogen heteroatoms. In some embodiments, Ring A is a 13-membered saturated or partially unsaturated tricyclic heteroarylenyl having 3 nitrogen heteroatoms.

In some embodiments, Ring A is a 5,6,6-fused saturated or partially unsaturated tricyclic heteroarylenyl having 1-5 heteroatoms independently selected from nitrogen, oxygen, and sulfur. In some embodiments, Ring A is a 5,6,6-fused membered saturated or partially unsaturated tricyclic heteroarylenyl having 1-5 nitrogen heteroatoms. In some embodiments, Ring A is a 5,6,6-fused membered saturated or partially unsaturated tricyclic heteroarylenyl having 3 nitrogen heteroatoms.

In some embodiments, Ring A is a 6,5,6-fused saturated or partially unsaturated tricyclic heteroarylenyl having 1-5 heteroatoms independently selected from nitrogen, oxygen, and sulfur. In some embodiments, Ring A is a 6,5,6-fused membered saturated or partially unsaturated tricyclic heteroarylenyl having 1-5 nitrogen heteroatoms. In some embodiments, Ring A is a 6,5,6-fused membered saturated or partially unsaturated tricyclic heteroarylenyl having 3 nitrogen heteroatoms.

In some embodiments, Ring A is a 14-membered saturated or partially unsaturated tricyclic heteroarylenyl having 1-5 heteroatoms independently selected from nitrogen, oxygen, and sulfur.

In some embodiments, Ring A is of formula ABC-1 or ABC-2:

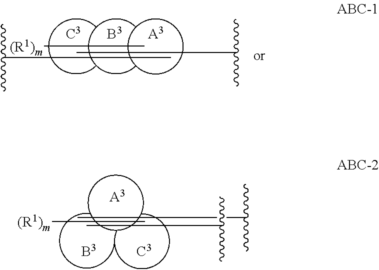

or a pharmaceutically acceptable salt thereof, wherein each R¹ and in is as defined above and described herein both individually and in combination; and:
each of Ring A³, Ring B³, and Ring C³ is independently a fused ring selected from a 3-7 membered saturated or partially unsaturated carbocyclylenyl; phenyl; a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

As defined above and described herein, each of Ring A³. Ring B³, and Ring C³ is independently a fused ring selected from a 3-7 membered saturated or partially unsaturated carbocyclylenyl: phenyl: a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

In some embodiments, Ring A³ is a fused 3-7 membered saturated or partially unsaturated carbocyclylenyl. In some embodiments, Ring A³ is a fused 4-7 membered saturated or partially unsaturated carbocyclylenyl. In some embodiments, Ring A³ is a fused 5-membered saturated or partially unsaturated carbocyclylenyl. In some embodiments, Ring A³ is a fused 6-membered saturated or partially unsaturated carbocyclylenyl. In some embodiments, Ring A³ is fused phenyl.

In some embodiments, Ring A³ is a fused 3-7 membered saturated or partially unsaturated heterocyclylenyl having 1-5 heteroatoms independently selected from nitrogen, oxygen, and sulfur. In some embodiments, Ring A³ is a fused 4-7 membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-2 nitrogen heteroatoms. In some embodiments, Ring A³ is a fused 5-membered saturated or partially unsaturated heterocyclylenyl having 1-5 heteroatoms independently selected from nitrogen, oxygen, and sulfur, and an oxo group. In some embodiments, Ring A³ is a fused 5-membered saturated or partially unsaturated heterocyclylenyl having 1-2 nitrogen heteroatoms, and an oxo group

In some embodiments, Ring A³ is a fused 6-membered saturated or partially unsaturated heterocyclylenyl having 1-5 heteroatoms independently selected from nitrogen, oxygen, and sulfur. In some embodiments, Ring A³ is a fused 6-membered saturated or partially unsaturated heterocyclylenyl having 1-2 nitrogen heteroatoms. In some embodiments, Ring A³ is a fused 6-membered saturated or partially unsaturated heterocyclylenyl having 1-5 heteroatoms independently selected from nitrogen, oxygen, and sulfur, and an oxo group. In some embodiments, Ring A³ is a fused 6-membered saturated or partially unsaturated heterocyclylenyl having 1-2 nitrogen heteroatoms, and an oxo group

In some embodiments, Ring A³ is a fused 5-6 membered heteroarylenyl having 1-5 heteroatoms independently selected from nitrogen, oxygen, and sulfur. In some embodiments, Ring A³ is a fused 5-membered heteroarylenyl having 1-5 heteroatoms independently selected from nitrogen, oxygen, and sulfur. In some embodiments, Ring A³ is a fused 6-membered heteroarylenyl having 1-5 heteroatoms independently selected from nitrogen, oxygen, and sulfur.

In some embodiments, Ring B³ is a fused 3-7 membered saturated or partially unsaturated carbocyclylenyl. In some embodiments, Ring B³ is a fused 4-7 membered saturated or partially unsaturated carbocyclylenyl. In some embodiments, Ring B³ is a fused 5-membered saturated or partially unsaturated carbocyclylenyl. In some embodiments, Ring B³ is a fused 6-membered saturated or partially unsaturated carbocyclylenyl. In some embodiments, Ring B³ is fused phenyl.

In some embodiments, Ring B³ is a fused 3-7 membered saturated or partially unsaturated heterocyclylenyl having 1-5 heteroatoms independently selected from nitrogen, oxygen, and sulfur. In some embodiments, Ring B³ is a fused 4-7 membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-2 nitrogen heteroatoms. In some embodiments, Ring B³ is a fused 5-membered saturated or partially unsaturated heterocyclylenyl having 1-5 heteroatoms independently selected from nitrogen, oxygen, and sulfur, and an oxo group. In some embodiments, Ring B³ is a fused 5-membered saturated or partially unsaturated heterocyclylenyl having 1-2 nitrogen heteroatoms, and an oxo group

In some embodiments, Ring B³ is a fused 6-membered saturated or partially unsaturated heterocyclylenyl having 1-5 heteroatoms independently selected from nitrogen, oxygen, and sulfur. In some embodiments, Ring B³ is a fused 6-membered saturated or partially unsaturated heterocyclylenyl having 1-2 nitrogen heteroatoms. In some embodiments, Ring B³ is a fused 6-membered saturated or partially unsaturated heterocyclylenyl having 1-5 heteroatoms independently selected from nitrogen, oxygen, and sulfur, and an oxo group. In some embodiments, Ring B³ is a fused 6-membered saturated or partially unsaturated heterocyclylenyl having 1-2 nitrogen heteroatoms, and an oxo group

In some embodiments, Ring B³ is a fused 5-6 membered heteroarylenyl having 1-5 heteroatoms independently selected from nitrogen, oxygen, and sulfur. In some embodiments, Ring B³ is a fused 5-membered heteroarylenyl having 1-5 heteroatoms independently selected from nitrogen, oxygen, and sulfur. In some embodiments, Ring B³ is a fused 6-membered heteroarylenyl having 1-5 heteroatoms independently selected from nitrogen, oxygen, and sulfur.

In some embodiments, Ring C³ is a fused 3-7 membered saturated or partially unsaturated carbocyclylenyl. In some embodiments, Ring C³ is a fused 4-7 membered saturated or partially unsaturated carbocyclylenyl. In some embodiments, Ring C³ is a fused 5-membered saturated or partially unsaturated carbocyclylenyl. In some embodiments, Ring C³ is a fused 6-membered saturated or partially unsaturated carbocyclylenyl. In some embodiments, Ring C³ is fused phenyl.

In some embodiments, Ring C³ is a fused 3-7 membered saturated or partially unsaturated heterocyclylenyl having 1-5 heteroatoms independently selected from nitrogen, oxygen, and sulfur. In some embodiments, Ring C³ is a fused 4-7 membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-2 nitrogen heteroatoms. In some embodiments, Ring C³ is a fused 5-membered saturated or partially unsaturated heterocyclylenyl having 1-5 heteroatoms independently selected from nitrogen, oxygen, and sulfur, and an oxo group. In some embodiments, Ring C³ is a fused 5-membered saturated or partially unsaturated heterocyclylenyl having 1-2 nitrogen heteroatoms, and an oxo group

In some embodiments, Ring C³ is a fused 6-membered saturated or partially unsaturated heterocyclylenyl having 1-5 heteroatoms independently selected from nitrogen, oxygen, and sulfur. In some embodiments, Ring C³ is a fused 6-membered saturated or partially unsaturated heterocyclylenyl having 1-2 nitrogen heteroatoms. In some embodiments, Ring C³ is a fused 6-membered saturated or partially unsaturated heterocyclylenyl having 1-5 heteroatoms independently selected from nitrogen, oxygen, and sulfur, and an oxo group. In some embodiments, Ring C³ is a fused 6-membered saturated or partially unsaturated heterocyclylenyl having 1-2 nitrogen heteroatoms, and an oxo group.

In some embodiments, Ring C³ is a fused 5-6 membered heteroarylenyl having 1-5 heteroatoms independently selected from nitrogen, oxygen, and sulfur. In some embodiments, Ring C³ is a fused 5-membered heteroarylenyl having 1-5 heteroatoms independently selected from nitrogen, oxygen, and sulfur. In some embodiments, Ring C³ is a fused 6-membered heteroarylenyl having 1-5 heteroatoms independently selected from nitrogen, oxygen, and sulfur.

In some embodiments, Ring A is of formula ABC-1 having a structure of formulae ABC-1a, ABC-1b, or ABC-1c:

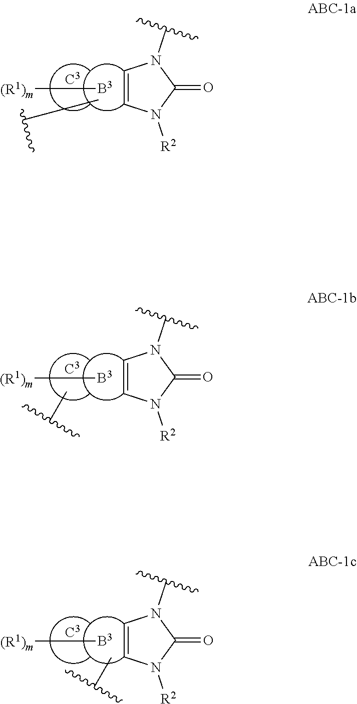

or a pharmaceutically acceptable salt thereof.

In some embodiments, Ring A is of formula ABC-1 having a structure of formulae ABC-1d, ABC-1e, or ABC-1f:

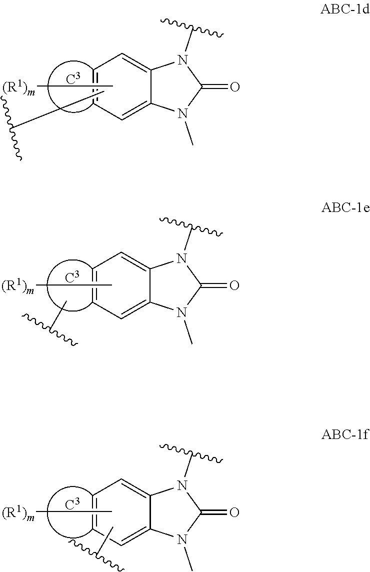

or a pharmaceutically acceptable salt thereof.

In some embodiments, Ring A is of formula ABC-1 having a structure of formulae ABC-1g, ABC-1h, ABC-1i, ABC-1j, ABC-1k, ABC-1l, ABC-1m, or ABC-1n:

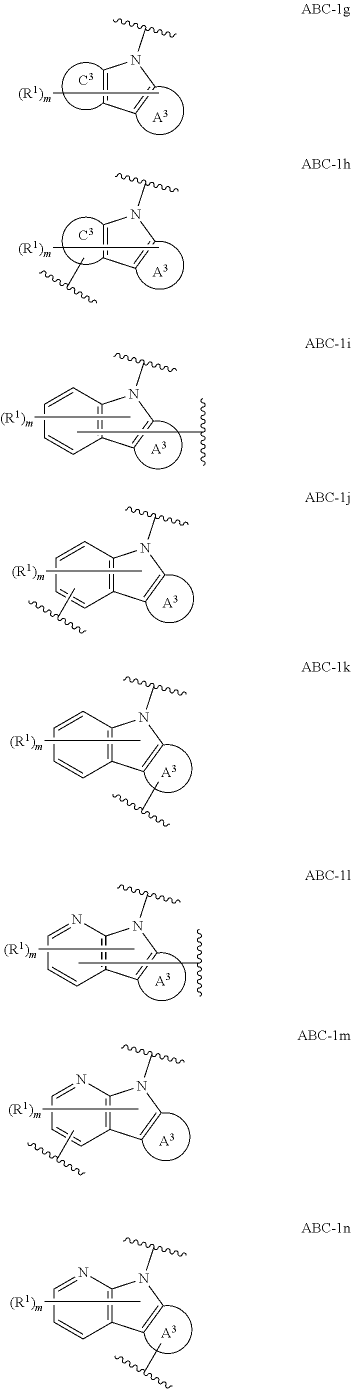

or a pharmaceutically acceptable salt thereof.

In some embodiments, Ring A is of formula ABC-2 having a structure of formulae ABC-2a, ABC-2b, ABC-2c, ABC-2d, ABC-2e, ABC-2f, or ABC-2g:

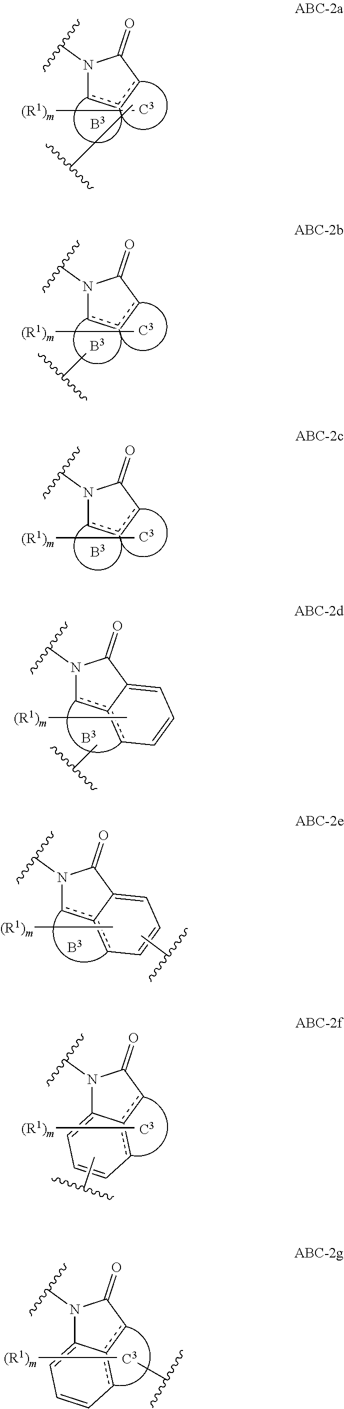

or a pharmaceutically acceptable salt thereof.

In some embodiments, Ring A is

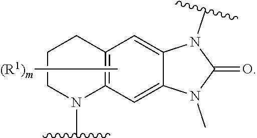

In some embodiments, Ring A is

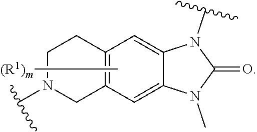

In some embodiments, Ring A is

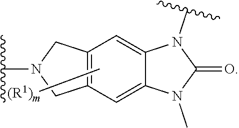

In some embodiments, Ring A is

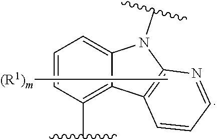

In some embodiments, Ring A is

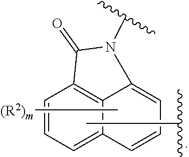

In some embodiments, Ring A is

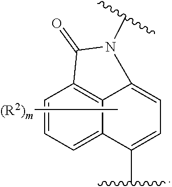

In some embodiments, Ring A, with its R¹ substituents is

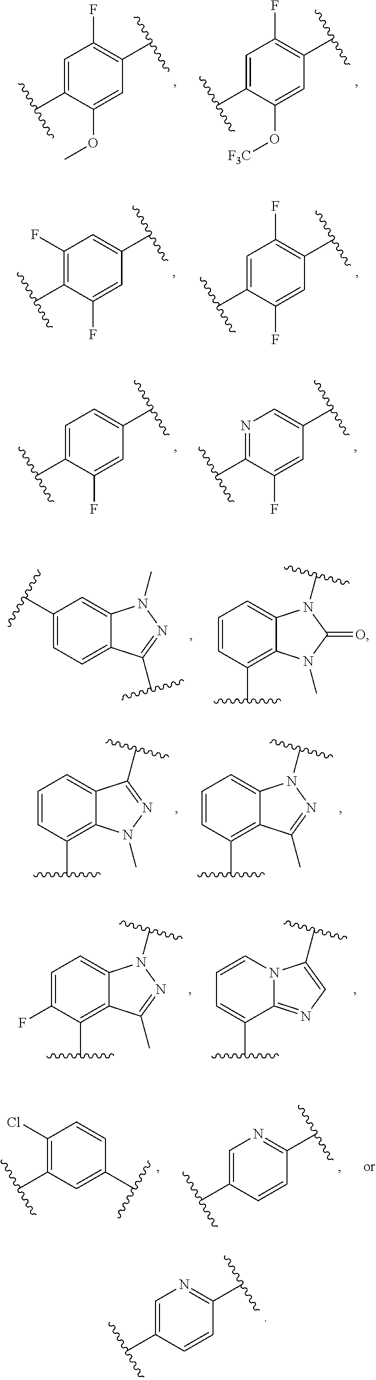

In some embodiments, Ring A, with its R¹ substituents and L¹ linker, is

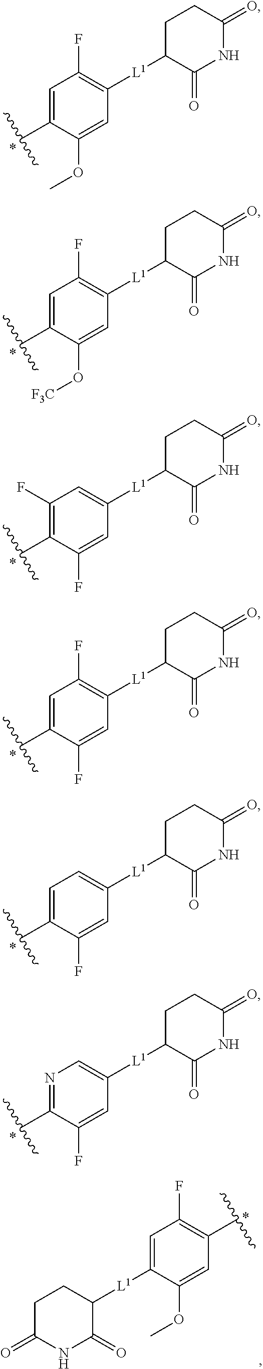

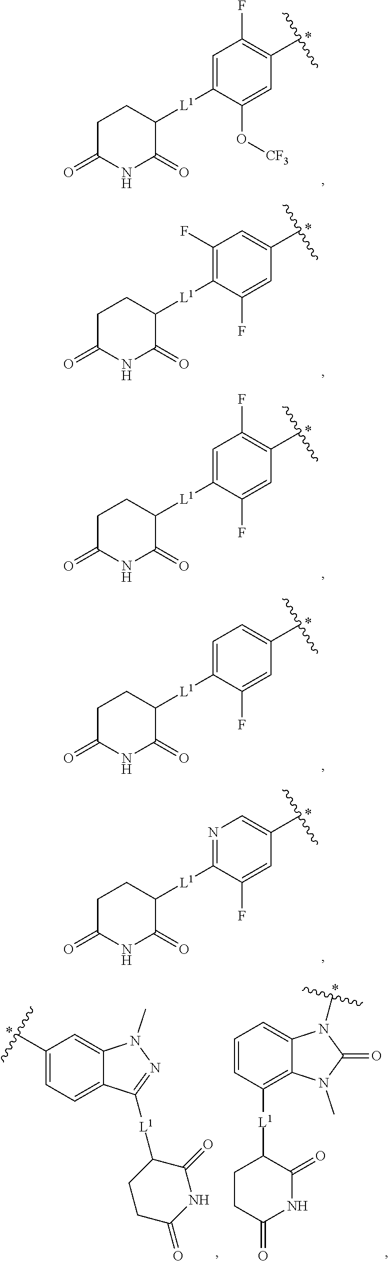

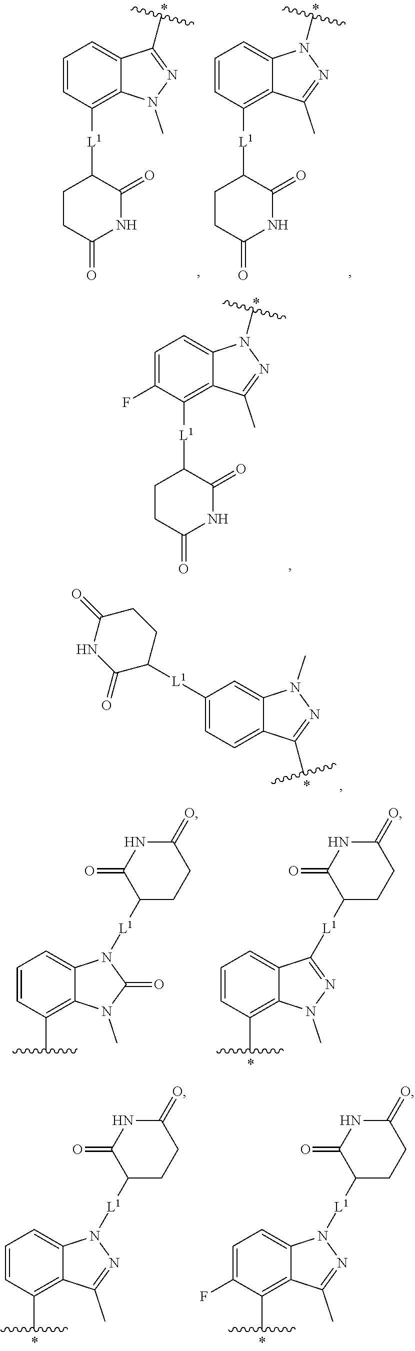

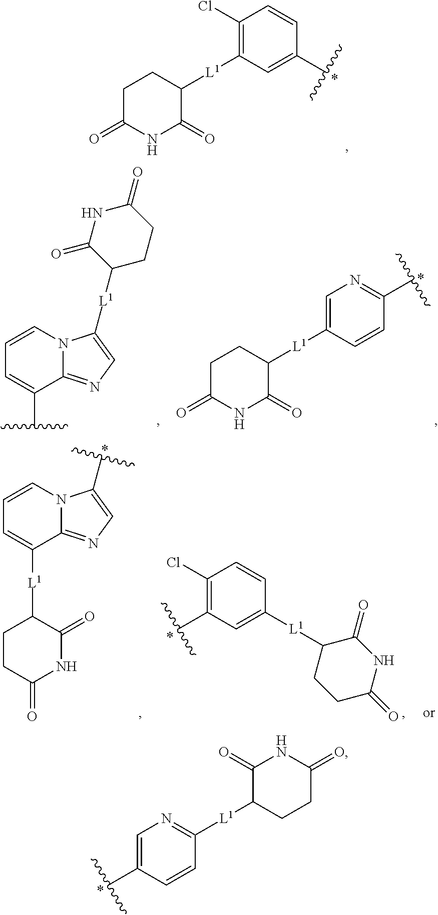

wherein

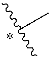

is the connection point to linker L.

In some embodiments, Ring A is selected from those depicted in the compounds of Table 1A below.

As defined above and described herein, Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur.

In some embodiments, Ring B is a fused ring selected from benzo or a 5-6 membered heteroaryl containing 1-4 heteroatoms independently selected from nitrogen, oxygen, and sulfur.

In some embodiments, Ring B is benzo. In some embodiments, Ring B is a 5-6 membered heteroaryl containing 1-4 heteroatoms independently selected from nitrogen, oxygen, and sulfur. In some embodiments, Ring B is pyridinyl.

In some embodiments, Ring B is a fused saturated or partially unsaturated 4-7 membered carbocyclyl. In some embodiments, Ring B is a fused saturated or partially unsaturated 4-6 membered carbocyclyl. In some embodiments, Ring B is a fused saturated or partially unsaturated 5-6 membered carbocyclyl. In some embodiments, Ring B is a saturated or partially unsaturated 4-7 membered heterocyclyl having 1-2 heteroatoms independently selected from nitrogen, oxygen, and sulfur. In some embodiments, Ring B is a saturated or partially unsaturated 4-6 membered heterocyclyl having 1-2 heteroatoms independently selected from nitrogen, oxygen, and sulfur. In some embodiments, Ring B is a saturated or partially unsaturated 5-6 membered heterocyclyl having 1-2 heteroatoms independently selected from nitrogen, oxygen, and sulfur.

In some embodiments, Ring B is selected from those depicted in the compounds of Table 1A below.

In some embodiments, Ring A and Ring B are

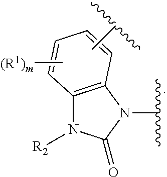

In some embodiments, Ring A and Ring B are

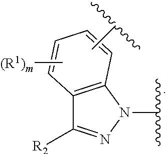

In some embodiments, Ring A and Ring B are

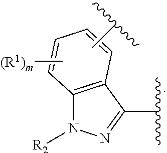

In some embodiments, Ring A and Ring B are

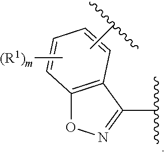

In some embodiments, Ring A and Ring B are

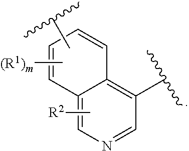

In some embodiments, LBM is

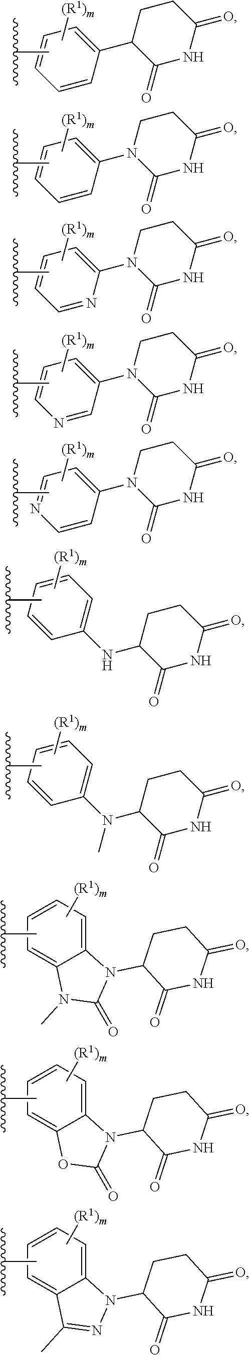

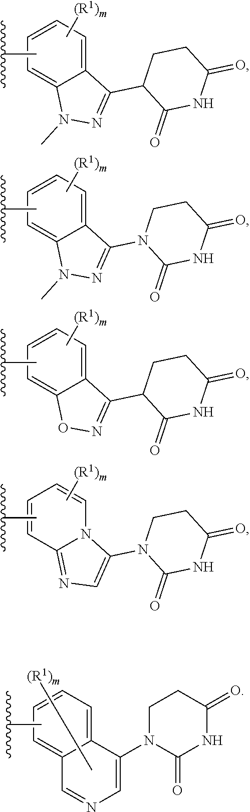

In some embodiments, LBM is

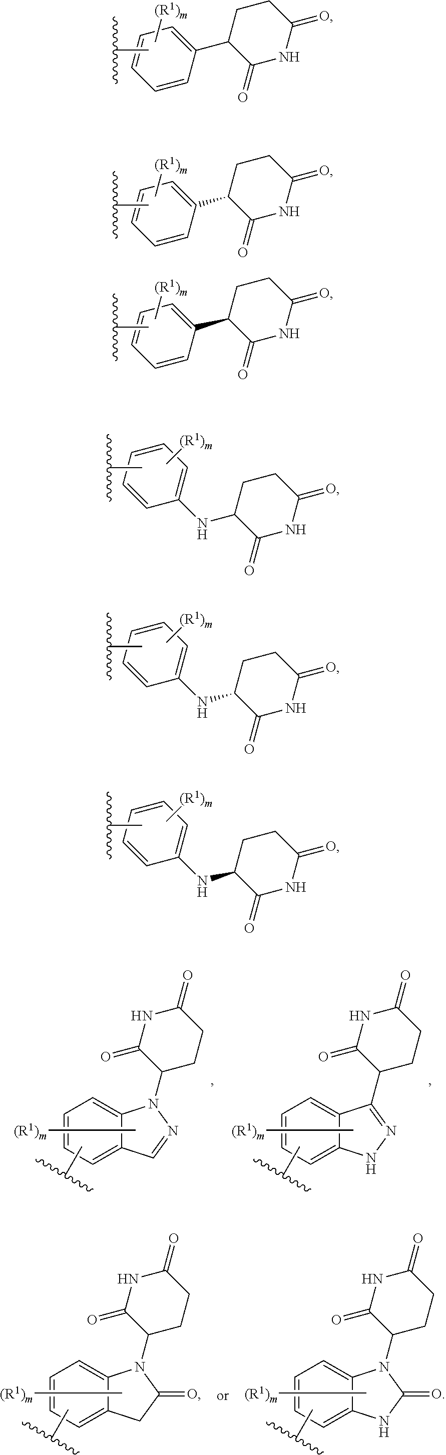

As defined above and described herein, R is independently hydrogen or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; or two R groups on the same or adjacent atoms or R and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur; or two R groups on the same carbon or nitrogen are taken together with their intervening atoms to form an optionally substituted 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 3-7 membered saturated or partially unsaturated carbocyclic ring. In some embodiments, R is an optionally substituted 3-7 membered heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur. In some embodiments, two R groups on the same carbon or nitrogen are taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur. In some embodiments, two R groups on the same carbon or nitrogen are taken together with their intervening atoms to form an optionally substituted 4-11 membered saturated or partially unsaturated monocyclic, bicyclic, bridged bicyclic, or spirocyclic carbocyclic. In some embodiments, two R groups on the same carbon or nitrogen are optionally taken together with their intervening atoms to form an optionally substituted heterocyclic ring having 0-3 heteroatoms, in addition to the carbon or nitrogen from which the two R groups are attached, independently selected from nitrogen, oxygen, and sulfur.

In some embodiments, R is C₁₋₆ alkyl (e.g., methyl, ethyl, isopropyl, etc.). In some embodiments, R is C₁₋₆ haloalkyl (e.g., —CF₃, CHF₂, etc.).

In some embodiments, R is selected from those depicted in the compounds of Table 1A below.

As defined above and described herein, m is 0, 1, 2, 3, 4, or 5. In some embodiments, m is 0. In some embodiments, m is 1. In some embodiments, m is 2. In some embodiments, m is 3. In some embodiments, m is 4. In some embodiments, in is 5. In some embodiments, in is 0 or 1. In some embodiments, in is 1 or 2. In some embodiments, m is 0, 1, or 2.

In some embodiments, m is selected from those depicted in the compounds of Table 1A below.

In some embodiments, LBM is a non-IMiD (immune modulatory drug), e.g., not thalidomide or a derivative thereof wherein Ring A is phthalimide.

In some embodiments, LBM is

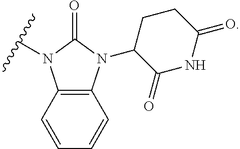 [SMILES: *n1c(=O)n(C2CCC(=O)NC2=O)c2ccccc21]

In some embodiments, LBM is

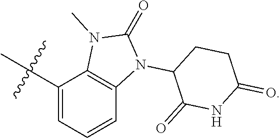 [SMILES: *c1cccc2c1n(C)c(=O)n2C1CCC(=O)NC1=O]

In some embodiments, LBM is

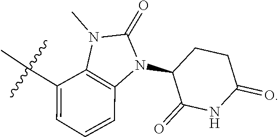 [SMILES: *c1cccc2c1n(C)c(=O)n2[C@H]1CCC(=O)NC1=O]

In some embodiments, LBM is

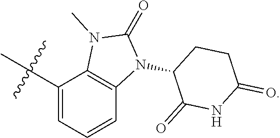 [SMILES: *c1cccc2c1n(C)c(=O)n2[C@@H]1CCC(=O)NC1=O]

In some embodiments, LBM is

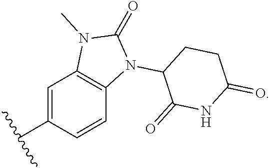 [SMILES: *c1ccc2c(c1)n(C)c(=O)n2C1CCC(=O)NC1=O]

In some embodiments, LBM is

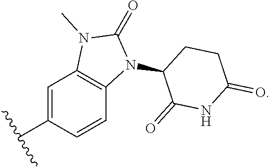 [SMILES: *c1ccc2c(c1)n(C)c(=O)n2[C@H]1CCC(=O)NC1=O]

In some embodiments, LBM is

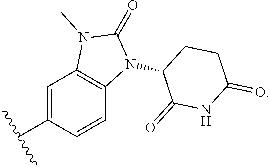 [SMILES: *c1ccc2c(c1)n(C)c(=O)n2[C@@H]1CCC(=O)NC1=O]

In some embodiments, LBM is

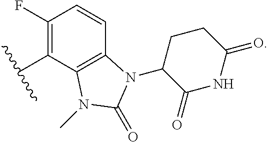 [SMILES: *c1c(F)ccc2c1n(C)c(=O)n2C1CCC(=O)NC1=O]

In some embodiments, LBM is

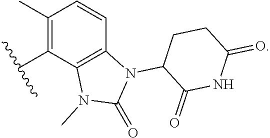 [SMILES: *c1c(C)ccc2c1n(C)c(=O)n2C1CCC(=O)NC1=O]

In some embodiments, LBM is

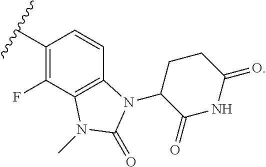 [SMILES: *c1ccc2c(c1F)n(C)c(=O)n2C1CCC(=O)NC1=O]

In some embodiments, LBM is

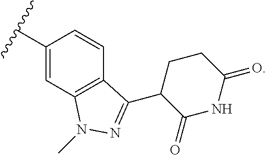 [SMILES: *c1ccc2c(C3CCC(=O)NC3=O)nn(C)c2c1]

In some embodiments, LBM is

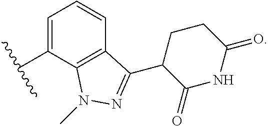 [SMILES: *c1cccc2c(C3CCC(=O)NC3=O)nn(C)c12]

In some embodiments, LBM is

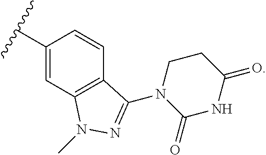 [SMILES: *c1ccc2c(N3CCC(=O)NC3=O)nn(C)c2c1]

In some embodiments, LBM is

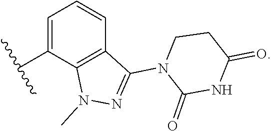 [SMILES: *c1cccc2c(N3CCC(=O)NC3=O)nn(C)c12]

In some embodiments, LBM is

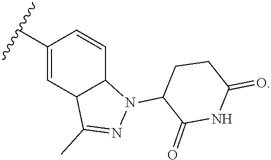 [SMILES: *C1=CC2C(C)=NN(C3CCC(=O)NC3=O)C2C=C1]

In some embodiments, LBM is

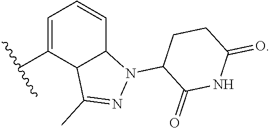 [SMILES: *C1=CC=CC2C1C(C)=NN2C1CCC(=O)NC1=O]

In some embodiments, LBM is

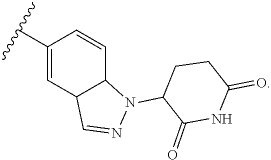 [SMILES: *C1=CC2C=NN(C3CCC(=O)NC3=O)C2C=C1]

In some embodiments, LBM is

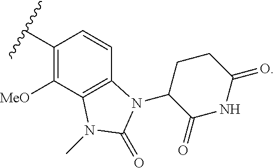 [SMILES: *c1ccc2c(c1OC)n(C)c(=O)n2C1CCC(=O)NC1=O]

In some embodiments, LBM is

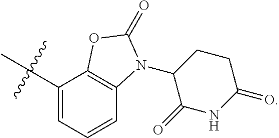 [SMILES: *c1cccc2c1oc(=O)n2C1CCC(=O)NC1=O]

In some embodiments, LBM is

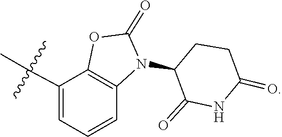 [SMILES: *c1cccc2c1oc(=O)n2[C@H]1CCC(=O)NC1=O]

In some embodiments, LBM is

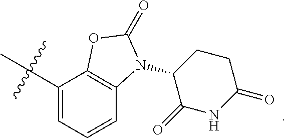 [SMILES: *c1cccc2c1oc(=O)n2[C@@H]1CCC(=O)NC1=O]

In some embodiments, LBM is

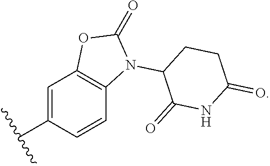 [SMILES: *c1ccc2c(c1)oc(=O)n2C1CCC(=O)NC1=O]

In some embodiments, LBM is

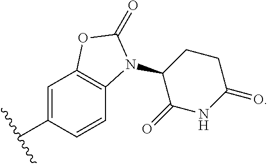 [SMILES: *c1ccc2c(c1)oc(=O)n2[C@H]1CCC(=O)NC1=O]

In some embodiments, LBM is

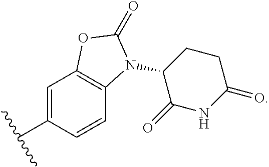 [SMILES: *c1ccc2c(c1)oc(=O)n2[C@@H]1CCC(=O)NC1=O]

In some embodiments, LBM is

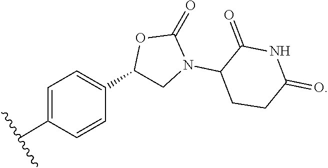 [SMILES: *c1ccc([C@H]2CN(C3CCC(=O)NC3=O)C(=O)O2)cc1]

In some embodiments, LBM is

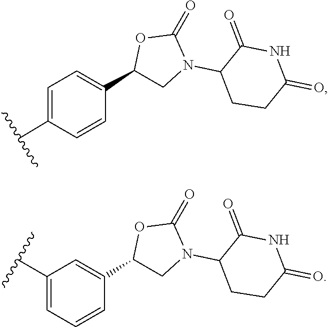 [SMILES: *c1ccc([C@@H]2CN(C3CCC(=O)NC3=O)C(=O)O2)cc1 ; *c1cccc([C@H]2CN(C3CCC(=O)NC3=O)C(=O)O2)c1]

In some embodiments, LBM is

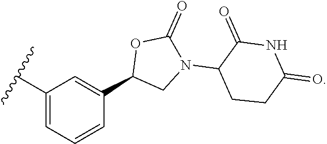 [SMILES: *c1cccc([C@@H]2CN(C3CCC(=O)NC3=O)C(=O)O2)c1]

In some embodiments, LBM is

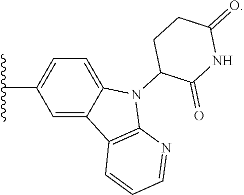 [SMILES: *c1ccc2c(c1)c1cccnc1n2C1CCC(=O)NC1=O]

In some embodiments, LBM is

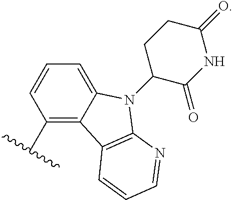 [SMILES: *c1cccc2c1c1cccnc1n2C1CCC(=O)NC1=O]

In some embodiments, LBM is

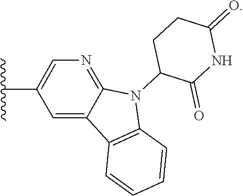 [SMILES: *c1cnc2c(c1)c1ccccc1n2C1CCC(=O)NC1=O]

In some embodiments, LBM is

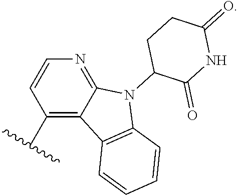 [SMILES: *c1ccnc2c1c1ccccc1n2C1CCC(=O)NC1=O]

In some embodiments, LBM is

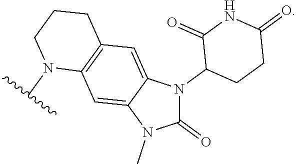 [SMILES: *N1CCCc2cc3c(cc21)n(C)c(=O)n3C1CCC(=O)NC1=O]

In some embodiments, LBM is

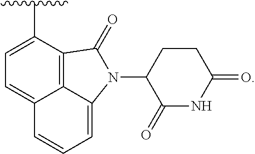 [SMILES: *c1ccc2cccc3c2c1C(=O)N3C1CCC(=O)NC1=O]

In some embodiments, LBM is

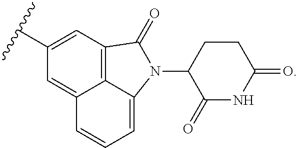 [SMILES: *c1cc2c3c(cccc3c1)N(C1CCC(=O)NC1=O)C2=O]

In some embodiments, LBM is

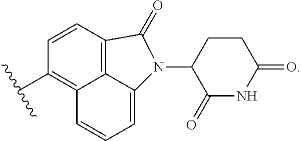 [SMILES: *c1ccc2c3c(cccc13)N(C1CCC(=O)NC1=O)C2=O]

In some embodiments, LBM is

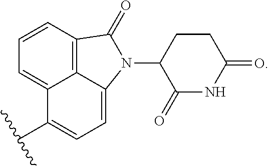 [SMILES: *c1ccc2c3c(cccc13)C(=O)N2C1CCC(=O)NC1=O]

In some embodiments, LBM is

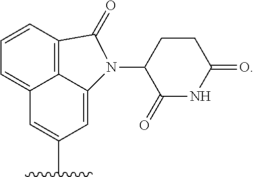 [SMILES: *c1cc2c3c(cccc3c1)C(=O)N2C1CCC(=O)NC1=O]

In some embodiments, LBM is

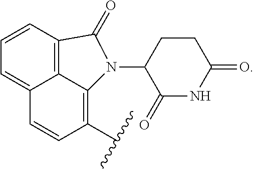 [SMILES: *c1ccc2cccc3c2c1N(C1CCC(=O)NC1=O)C3=O]

In some embodiments, LBM is

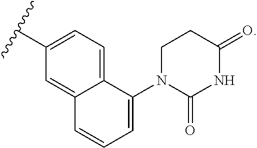 [SMILES: *c1ccc2c(N3CCC(=O)NC3=O)cccc2c1]

In some embodiments, LBM is

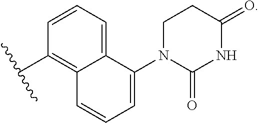 [SMILES: *c1cccc2c(N3CCC(=O)NC3=O)cccc12]

In some embodiments, LBM is

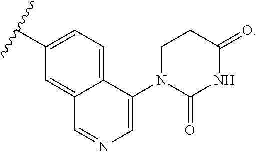 [SMILES: *c1ccc2c(N3CCC(=O)NC3=O)cncc2c1]

In some embodiments, LBM is

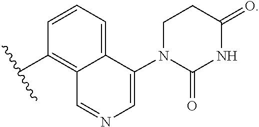 [SMILES: *c1cccc2c(N3CCC(=O)NC3=O)cncc12]

In some embodiments, LBM is

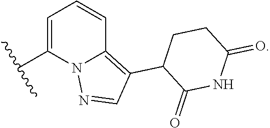 [SMILES: *c1cccc2c(C3CCC(=O)NC3=O)cnn12]

In some embodiments, LBM is

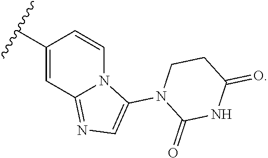 [SMILES: *c1ccn2c(N3CCC(=O)NC3=O)cnc2c1]

In some embodiments, LBM is

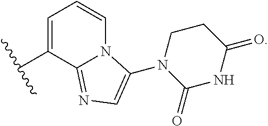 [SMILES: *c1cccn2c(N3CCC(=O)NC3=O)cnc12]

In some embodiments, LBM is

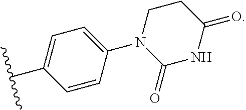 [SMILES: *c1ccc(N2CCC(=O)NC2=O)cc1]

In some embodiments, LBM is

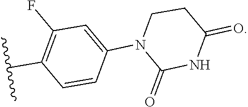 [SMILES: *c1ccc(N2CCC(=O)NC2=O)cc1F]

In some embodiments, LBM is

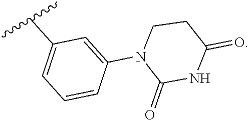 [SMILES: *c1cccc(N2CCC(=O)NC2=O)c1]

In some embodiments, LBM is

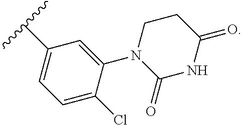 [SMILES: *c1ccc(Cl)c(N2CCC(=O)NC2=O)c1]

In some embodiments, LBM is

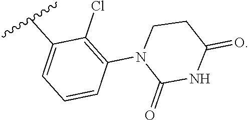 [SMILES: *c1cccc(N2CCC(=O)NC2=O)c1Cl]

In some embodiments, LBM is

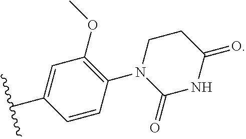 [SMILES: *c1ccc(N2CCC(=O)NC2=O)c(OC)c1]

In some embodiments, LBM is

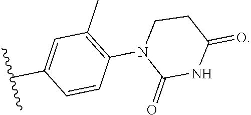 [SMILES: *c1ccc(N2CCC(=O)NC2=O)c(C)c1]

In some embodiments, LBM is

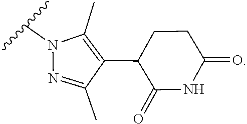 [SMILES: *n1nc(C)c(C2CCC(=O)NC2=O)c1C]

In some embodiments, LBM is

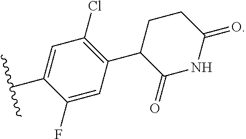 [SMILES: *c1cc(Cl)c(C2CCC(=O)NC2=O)cc1F]

In some embodiments, LBM is

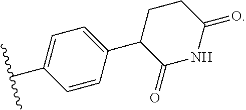 [SMILES: *c1ccc(C2CCC(=O)NC2=O)cc1]

In some embodiments, LBM is

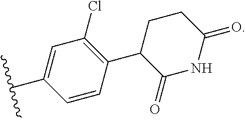 [SMILES: *c1ccc(C2CCC(=O)NC2=O)c(Cl)c1]

In some embodiments, LBM is

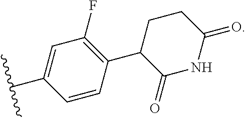 [SMILES: *c1ccc(C2CCC(=O)NC2=O)c(F)c1]

In some embodiments, LBM is

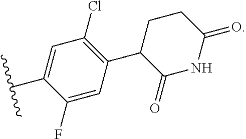 [SMILES: *c1cc(Cl)c(C2CCC(=O)NC2=O)cc1F]

In some embodiments, LBM is

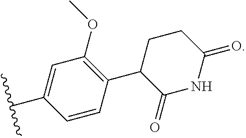 [SMILES: *c1ccc(C2CCC(=O)NC2=O)c(OC)c1]

In some embodiments, LBM is

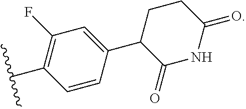 [SMILES: *c1ccc(C2CCC(=O)NC2=O)cc1F]

In some embodiments, LBM is

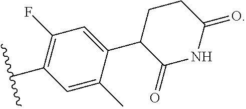 [SMILES: *c1cc(C)c(C2CCC(=O)NC2=O)cc1F]

In some embodiments, LBM is

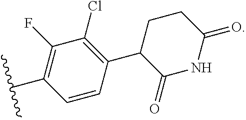 [SMILES: *c1ccc(C2CCC(=O)NC2=O)c(Cl)c1F]

In some embodiments, LBM is

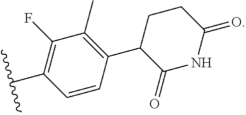 [SMILES: *c1ccc(C2CCC(=O)NC2=O)c(C)c1F]

In some embodiments, LBM is

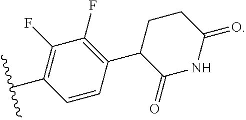 [SMILES: *c1ccc(C2CCC(=O)NC2=O)c(F)c1F]

In some embodiments, LBM is

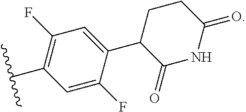 [SMILES: *c1cc(F)c(C2CCC(=O)NC2=O)cc1F]

In some embodiments, LBM is

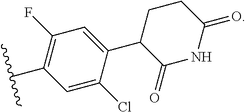 [SMILES: *c1cc(Cl)c(C2CCC(=O)NC2=O)cc1F]

In some embodiments, LBM is

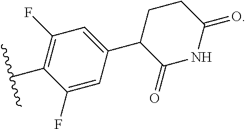 [SMILES: *c1c(F)cc(C2CCC(=O)NC2=O)cc1F]

In some embodiments, LBM is

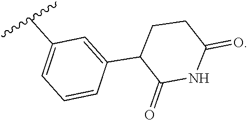 [SMILES: *c1cccc(C2CCC(=O)NC2=O)c1]

In some embodiments, LBM is

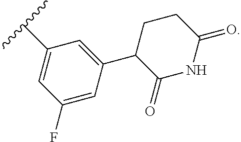 [SMILES: *c1cc(F)cc(C2CCC(=O)NC2=O)c1]

In some embodiments, LBM is

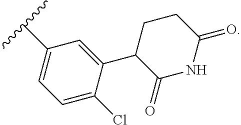 [SMILES: *c1ccc(Cl)c(C2CCC(=O)NC2=O)c1]

In some embodiments, LBM is

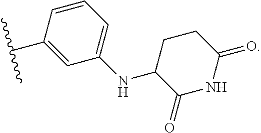 [SMILES: *c1cccc(NC2CCC(=O)NC2=O)c1]

In some embodiments, LBM is

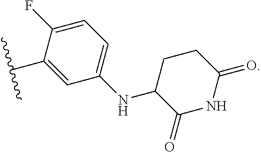 [SMILES: *c1cc(NC2CCC(=O)NC2=O)ccc1F]

In some embodiments, LBM is

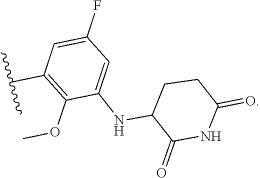 [SMILES: *c1cc(F)cc(NC2CCC(=O)NC2=O)c1OC]

In some embodiments, LBM is

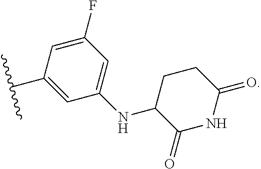 [SMILES: *c1cc(F)cc(NC2CCC(=O)NC2=O)c1]

In some embodiments, LBM is

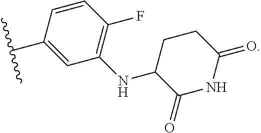 [SMILES: *c1ccc(F)c(NC2CCC(=O)NC2=O)c1]

In some embodiments, LBM is

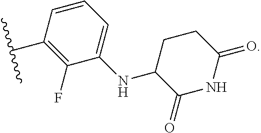 [SMILES: *c1cccc(NC2CCC(=O)NC2=O)c1F]

In some embodiments, LBM is

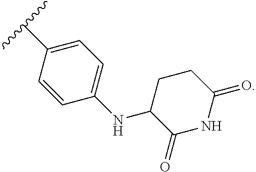 [SMILES: *c1ccc(NC2CCC(=O)NC2=O)cc1]

In some embodiments, LBM is

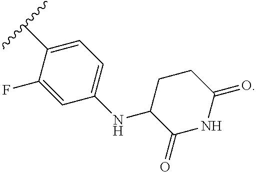 [SMILES: *c1ccc(NC2CCC(=O)NC2=O)cc1F]

In some embodiments, LBM is

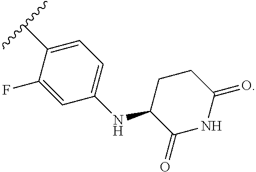 [SMILES: *c1ccc(N[C@H]2CCC(=O)NC2=O)cc1F]

In some embodiments, LBM is

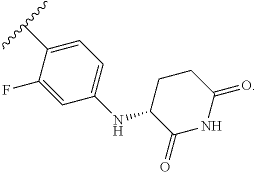 [SMILES: *c1ccc(N[C@@H]2CCC(=O)NC2=O)cc1F]

In some embodiments, LBM is

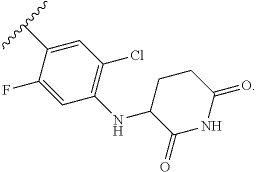 [SMILES: *c1cc(Cl)c(NC2CCC(=O)NC2=O)cc1F]

In some embodiments, LBM is

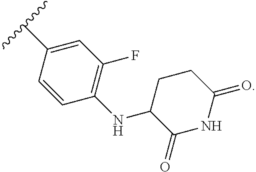 [SMILES: *c1ccc(NC2CCC(=O)NC2=O)c(F)c1]

In some embodiments, LBM is

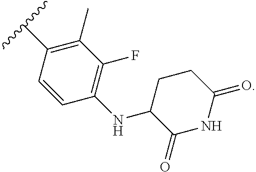 [SMILES: *c1ccc(NC2CCC(=O)NC2=O)c(F)c1C]

In some embodiments, LBM is

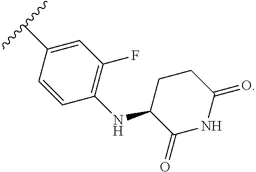 [SMILES: *c1ccc(N[C@H]2CCC(=O)NC2=O)c(F)c1]

In some embodiments, LBM is

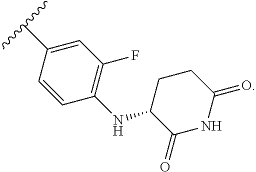 [SMILES: *c1ccc(N[C@@H]2CCC(=O)NC2=O)c(F)c1]

In some embodiments, LBM is

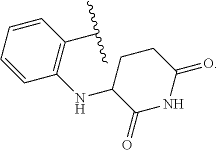 [SMILES: *c1ccccc1NC1CCC(=O)NC1=O]

In some embodiments, LBM is

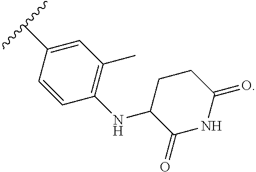 [SMILES: *c1ccc(NC2CCC(=O)NC2=O)c(C)c1]

In some embodiments, LBM is

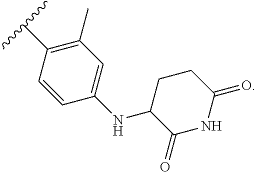 [SMILES: *c1ccc(NC2CCC(=O)NC2=O)cc1C]

In some embodiments, LBM is

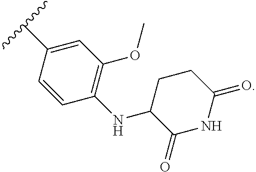 [SMILES: *c1ccc(NC2CCC(=O)NC2=O)c(OC)c1]

In some embodiments, LBM is

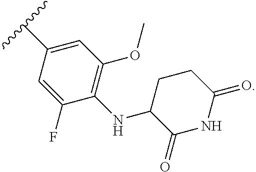 [SMILES: *c1cc(F)c(NC2CCC(=O)NC2=O)c(OC)c1]

In some embodiments, LBM is

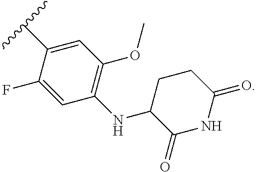 [SMILES: *c1cc(OC)c(NC2CCC(=O)NC2=O)cc1F]

In some embodiments, LBM is

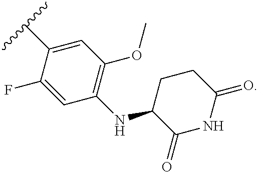 [SMILES: *c1cc(OC)c(N[C@H]2CCC(=O)NC2=O)cc1F]

In some embodiments, LBM is

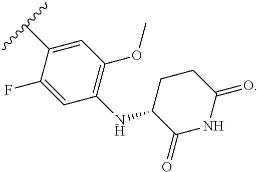 [SMILES: *c1cc(OC)c(N[C@@H]2CCC(=O)NC2=O)cc1F]

In some embodiments, LBM is

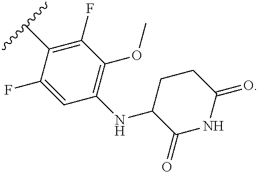 [SMILES: *c1c(F)cc(NC2CCC(=O)NC2=O)c(OC)c1F]

In some embodiments, LBM is

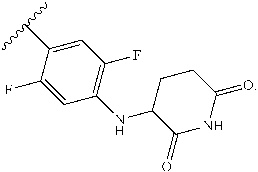 [SMILES: *c1cc(F)c(NC2CCC(=O)NC2=O)cc1F]

In some embodiments, LBM is

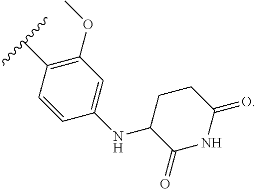 [SMILES: *c1ccc(NC2CCC(=O)NC2=O)cc1OC]

In some embodiments, LBM is

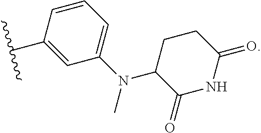 [SMILES: *c1cccc(N(C)C2CCC(=O)NC2=O)c1]

In some embodiments, LBM is

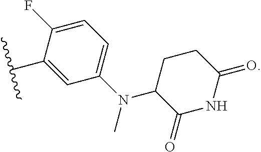 [SMILES: *c1cc(N(C)C2CCC(=O)NC2=O)ccc1F]

In some embodiments, LBM is

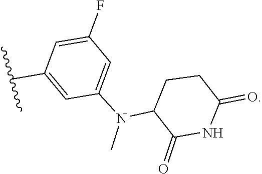 [SMILES: *c1cc(F)cc(N(C)C2CCC(=O)NC2=O)c1]

In some embodiments, LBM is

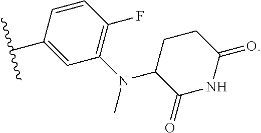 [SMILES: *c1ccc(F)c(N(C)C2CCC(=O)NC2=O)c1]

In some embodiments, LBM is

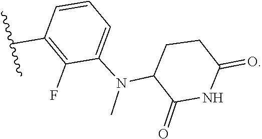 [SMILES: *c1cccc(N(C)C2CCC(=O)NC2=O)c1F]

In some embodiments, LBM is

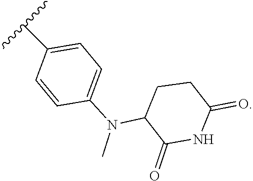 [SMILES: *c1ccc(N(C)C2CCC(=O)NC2=O)cc1]

In some embodiments, LBM is

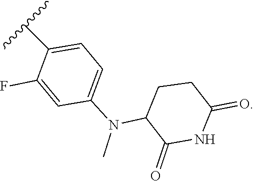 [SMILES: *c1ccc(N(C)C2CCC(=O)NC2=O)cc1F]

In some embodiments, LBM is

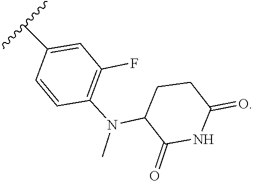 [SMILES: *c1ccc(N(C)C2CCC(=O)NC2=O)c(F)c1]

In some embodiments, LBM is

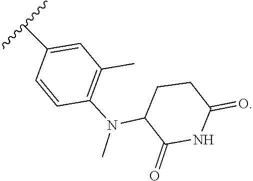 [SMILES: *c1ccc(N(C)C2CCC(=O)NC2=O)c(C)c1]

In some embodiments, LBM is

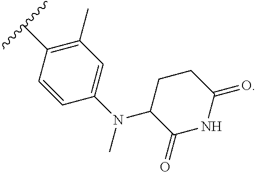 [SMILES: *c1ccc(N(C)C2CCC(=O)NC2=O)cc1C]

In some embodiments, LBM is

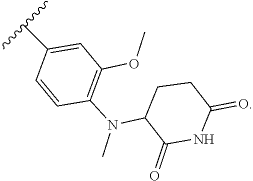 [SMILES: *c1ccc(N(C)C2CCC(=O)NC2=O)c(OC)c1]

In some embodiments, LBM is

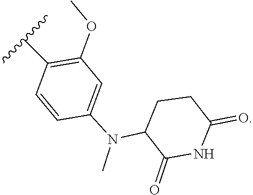 [SMILES: *c1ccc(N(C)C2CCC(=O)NC2=O)cc1OC]

In some embodiments, LBM is

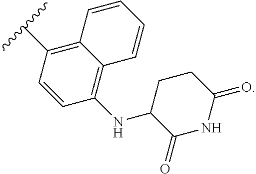 [SMILES: *c1ccc(NC2CCC(=O)NC2=O)c2ccccc12]

In some embodiments, LBM is

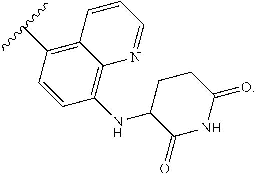 [SMILES: *c1ccc(NC2CCC(=O)NC2=O)c2ncccc12]

In some embodiments, LBM is

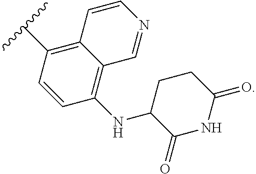 [SMILES: *c1ccc(NC2CCC(=O)NC2=O)c2cnccc12]

In some embodiments, LBM is

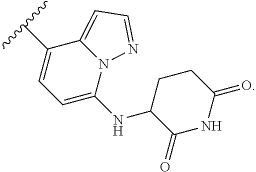 [SMILES: *c1ccc(NC2CCC(=O)NC2=O)n2nccc12]

In some embodiments, LBM is

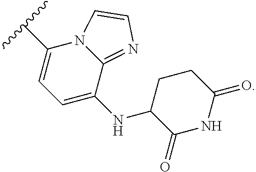 [SMILES: *c1ccc(NC2CCC(=O)NC2=O)c2nccn12]

In some embodiments, LBM is

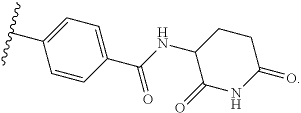 [SMILES: *c1ccc(C(=O)NC2CCC(=O)NC2=O)cc1]

In some embodiments, LBM is

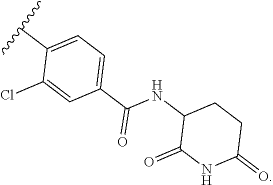 [SMILES: *c1ccc(C(=O)NC2CCC(=O)NC2=O)cc1Cl]

In some embodiments, LBM is

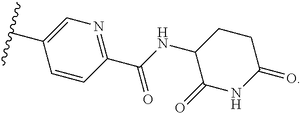 [SMILES: *c1ccc(C(=O)NC2CCC(=O)NC2=O)nc1]

In some embodiments, LBM is

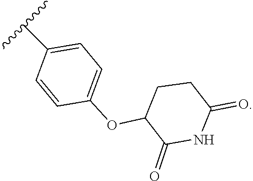 [SMILES: *c1ccc(OC2CCC(=O)NC2=O)cc1]

In some embodiments, LBM is

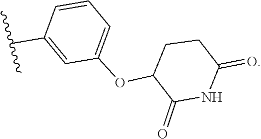 [SMILES: *c1cccc(OC2CCC(=O)NC2=O)c1]

In some embodiments, LBM is

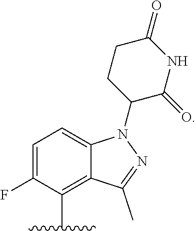 [SMILES: *c1c(F)ccc2c1c(C)nn2C1CCC(=O)NC1=O]

In some embodiments, LBM is

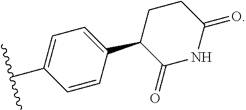 [SMILES: *c1ccc([C@H]2CCC(=O)NC2=O)cc1]

In some embodiments, LBM is

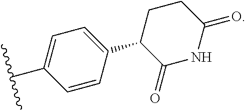 [SMILES: *c1ccc([C@@H]2CCC(=O)NC2=O)cc1]

In some embodiments, LBM is

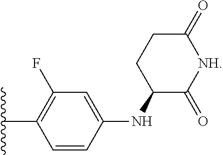 [SMILES: *c1ccc(N[C@H]2CCC(=O)NC2=O)cc1F]

In some embodiments, LBM is

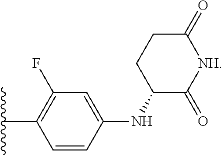 [SMILES: *c1ccc(N[C@@H]2CCC(=O)NC2=O)cc1F]

In certain embodiments, the present invention provides methods of degrading STAT6, or a mutant thereof, or methods of treating a STAT6-mediated disease, disorder, or condition, comprising administering a compound of formula I-aa-1 as a compound of any one of the following formulae:

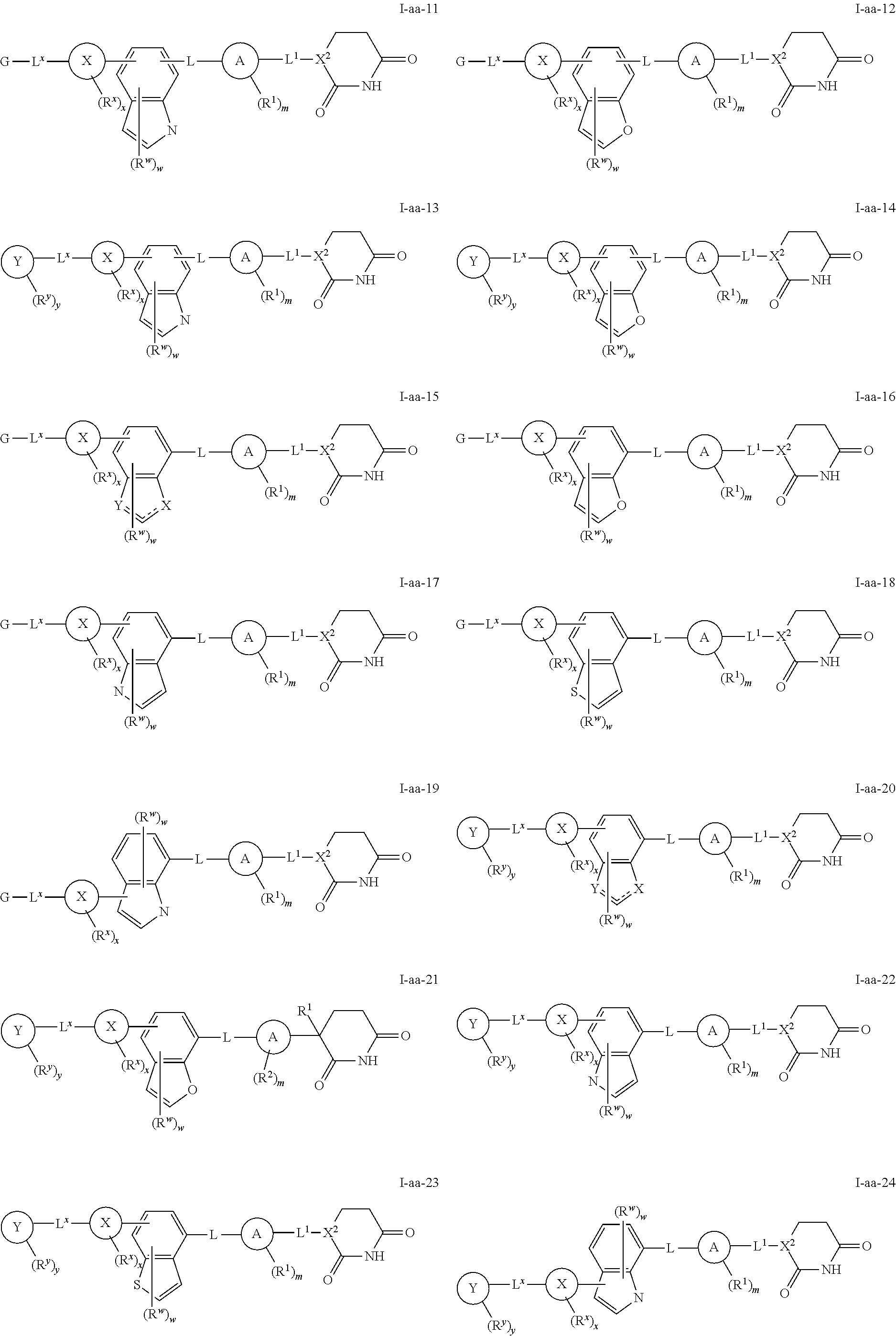

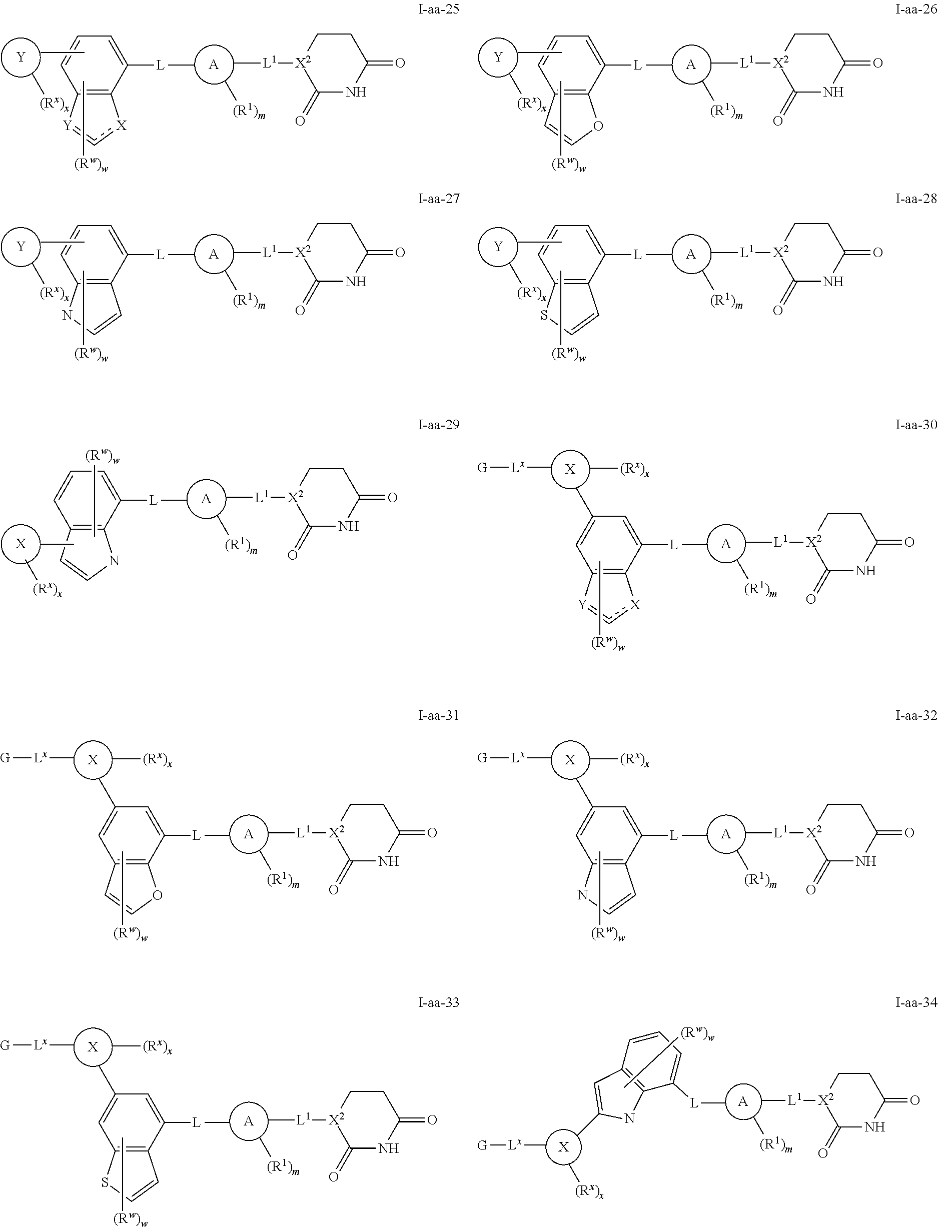

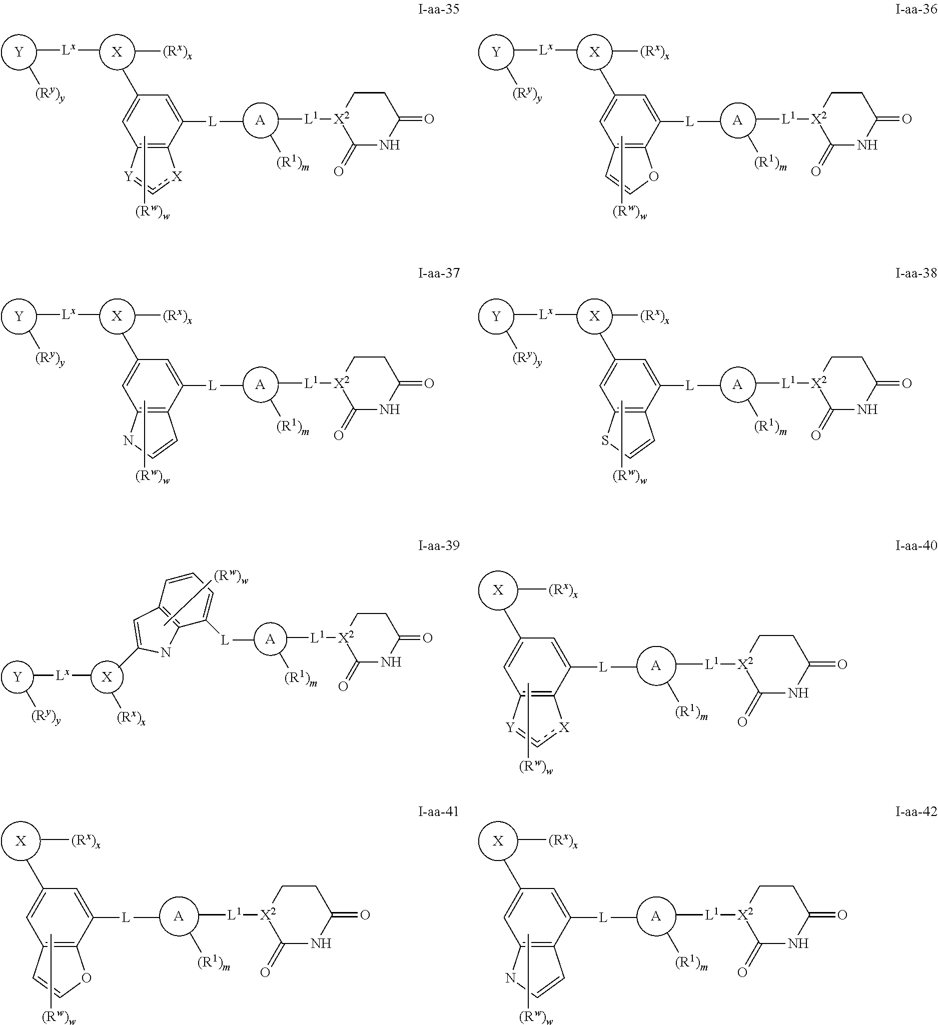

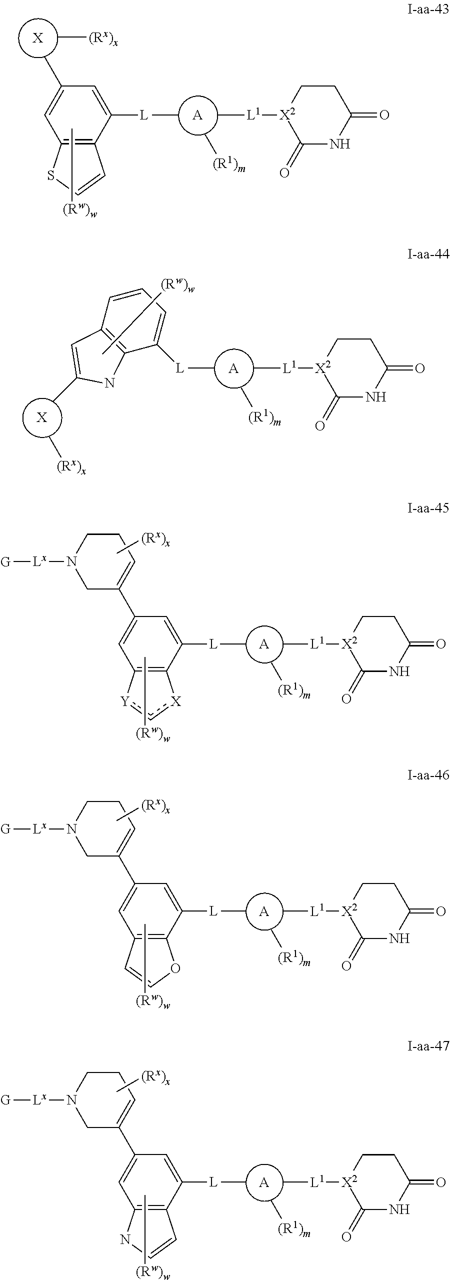

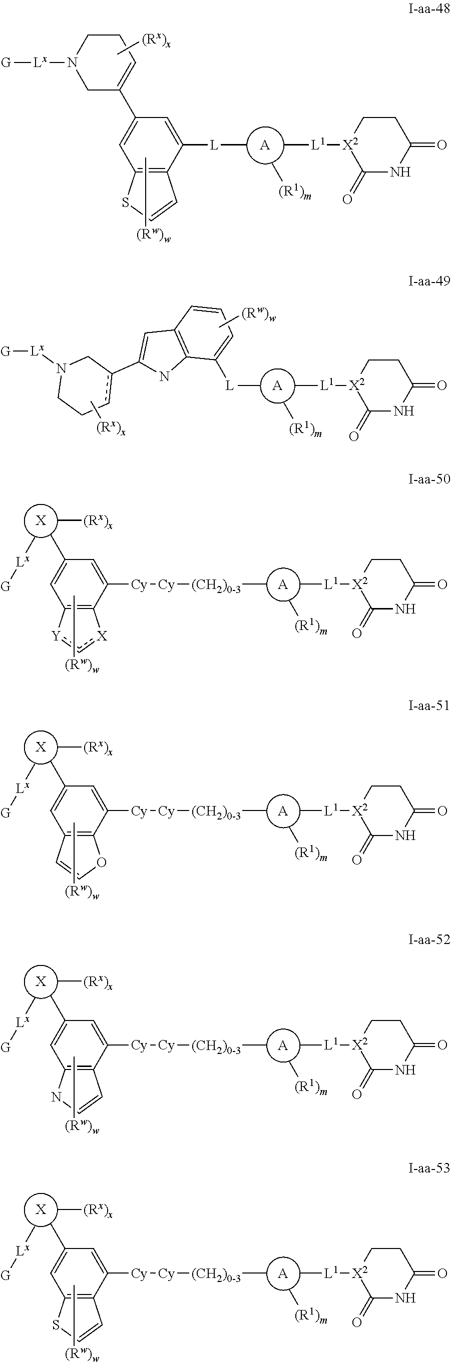

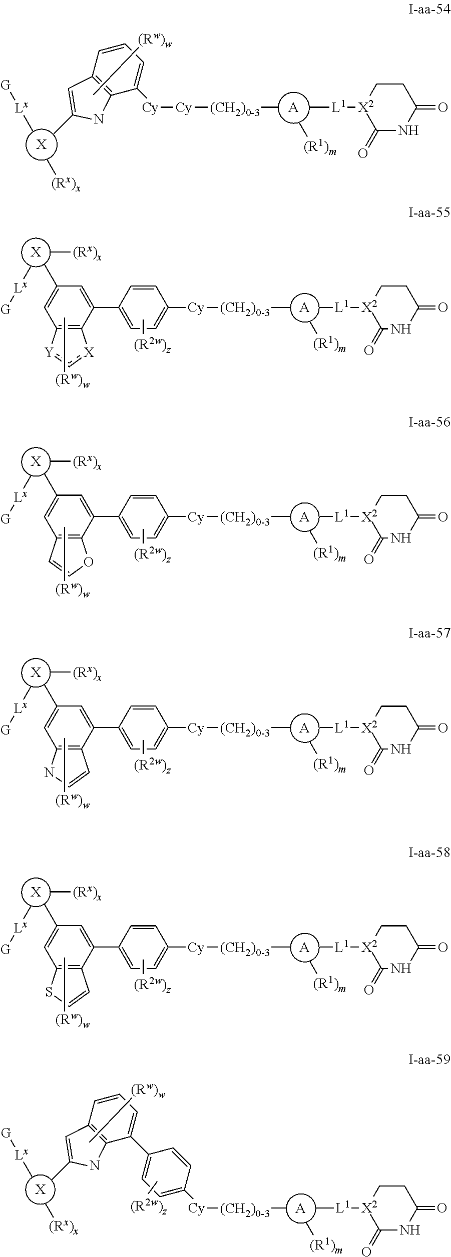

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition, comprising administering a compound of formula I, wherein LBM is an E3 ubiquitin ligase (cereblon) binding moiety thereby forming a compound of formula I-oo-1, I-oo-2, I-oo-3, I-oo-4, I-oo-5, I-oo-6, I-oo-7, I-oo-8, I-oo-9, or I-oo-10 respectively:

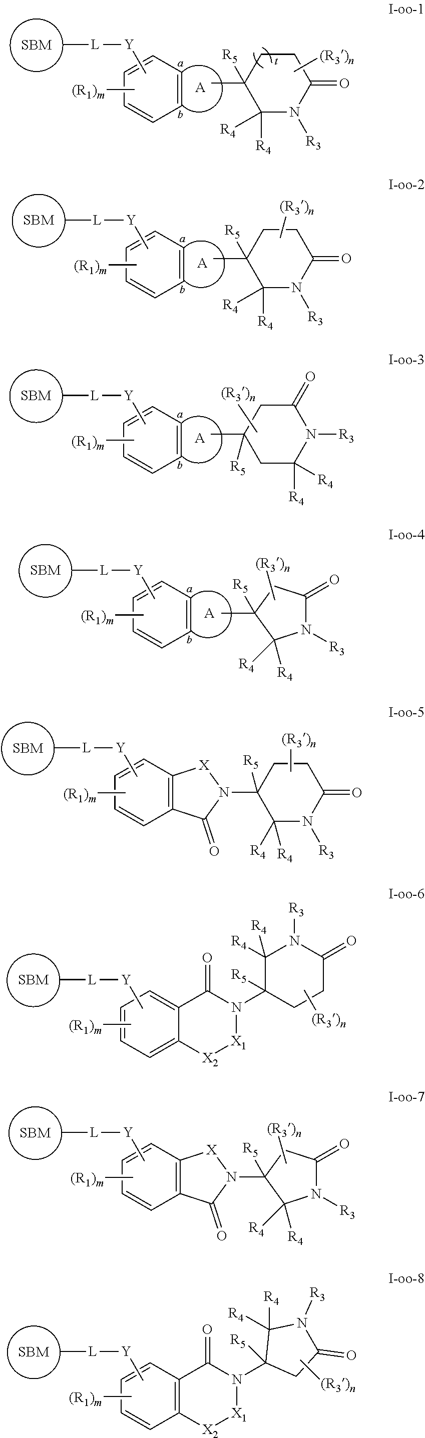

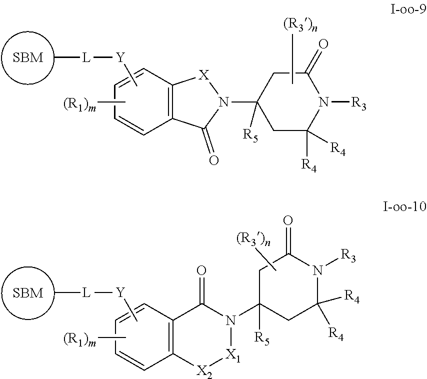

or a compound of formula I-oo′-1, I-oo′-2, I-oo′-3, I-oo′-4, I-oo′-5, I-oo′-6, I-oo′-7, I-oo′-8, I-oo′-9, or I-oo-10 respectively:

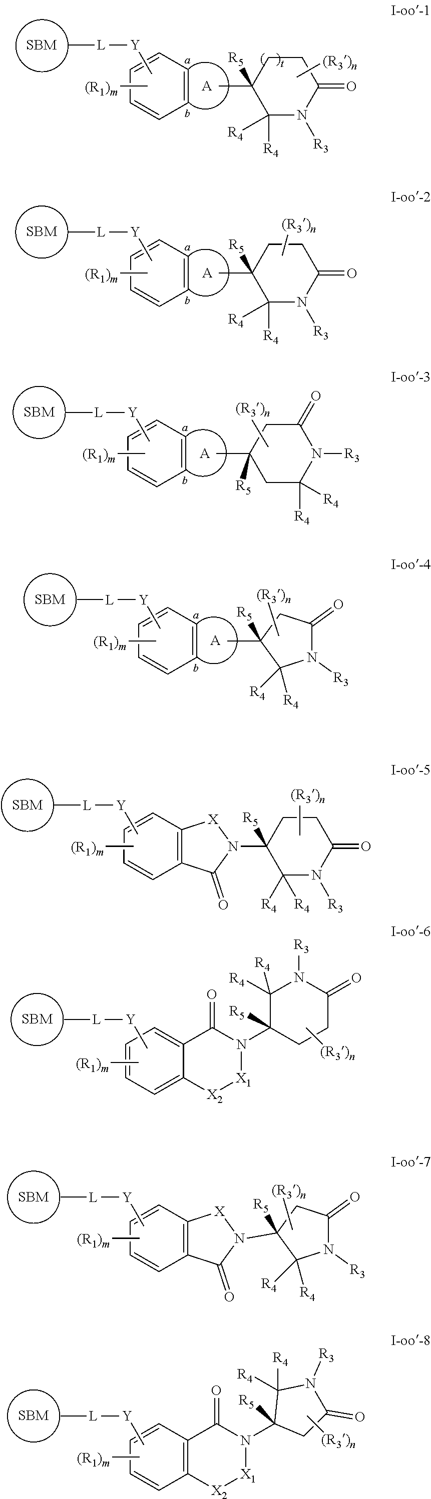

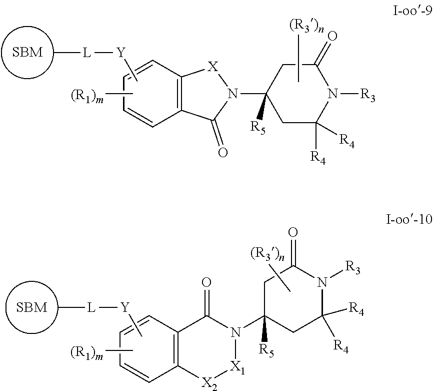

or a compound of formula I-oo″-1, I-oo″-2, I-oo″-3, I-oo″-4, I-oo″-5. I-oo″-6, I-oo″-7. I-oo″-8, I-oo″-9, or I-oo″-10 respectively:

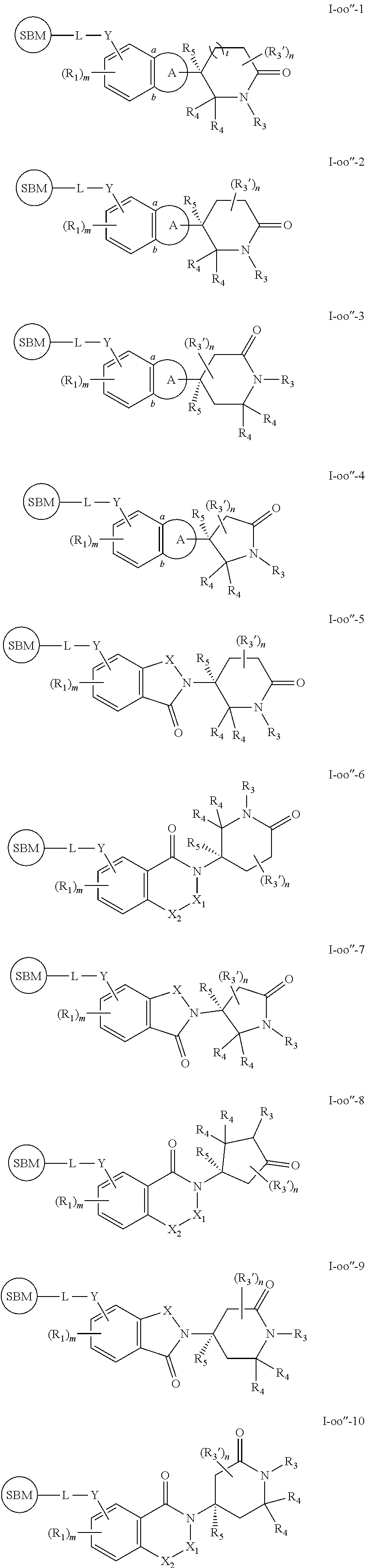

or a pharmaceutically acceptable salt thereof, wherein:

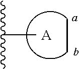

is

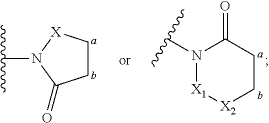

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene:
X is C(O) or C(R₃)₂;
X₁-X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆alkenyl, C(O)—C₃-C₈ cycloalkyl, or C(O)-3- to 8-membered heterocycloalkyl, and R₂ is optionally substituted with one or more of halogen, N(Rₐ)₂, NHC(O)R, NHC(O)ORₐ, OR_{b}, C₃-C₈ cycloalkyl, 3- to 8-membered heterocycloalkyl, C₆-C₁₀ aryl, or a 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆alkoxy, or C₁-C₆haloalkoxy;
R₂′ is H, C₁-C₆ alkyl, C₂-C₆ alkenyl, C₃-C₅ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In some embodiments, LBM is

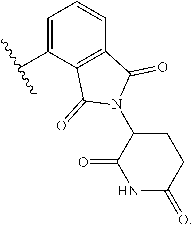 [SMILES: *c1cccc2c1C(=O)N(C1CCC(=O)NC1=O)C2=O]

In some embodiments, LBM is

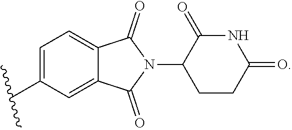 [SMILES: *c1ccc2c(c1)C(=O)N(C1CCC(=O)NC1=O)C2=O]

In some embodiments, LBM is

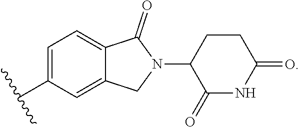 [SMILES: *c1ccc2c(c1)CN(C1CCC(=O)NC1=O)C2=O]

In some embodiments, LBM is

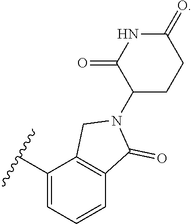 [SMILES: *c1cccc2c1CN(C1CCC(=O)NC1=O)C2=O]

In some embodiments, LBM is

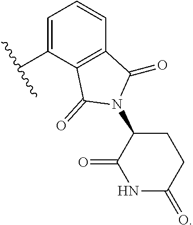 [SMILES: *c1cccc2c1C(=O)N([C@H]1CCC(=O)NC1=O)C2=O]

In some embodiments, LBM is

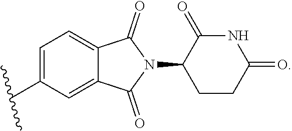 [SMILES: *c1ccc2c(c1)C(=O)N([C@@H]1CCC(=O)NC1=O)C2=O]

In some embodiments, LBM is

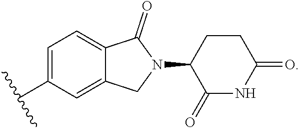 [SMILES: *c1ccc2c(c1)CN([C@H]1CCC(=O)NC1=O)C2=O]

In some embodiments, LBM is

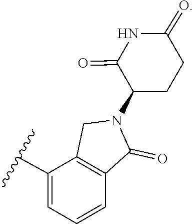 [SMILES: *c1cccc2c1CN([C@@H]1CCC(=O)NC1=O)C2=O]

In some embodiments, LBM is

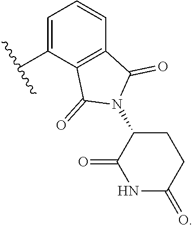 [SMILES: *c1cccc2c1C(=O)N([C@@H]1CCC(=O)NC1=O)C2=O]

In some embodiments, LBM is

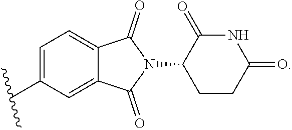 [SMILES: *c1ccc2c(c1)C(=O)N([C@H]1CCC(=O)NC1=O)C2=O]

In some embodiments, LBM is

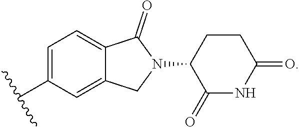 [SMILES: *c1ccc2c(c1)CN([C@@H]1CCC(=O)NC1=O)C2=O]

In some embodiments, LBM is

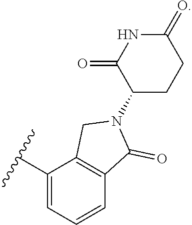 [SMILES: *c1cccc2c1CN([C@H]1CCC(=O)NC1=O)C2=O]

In some embodiments, LBM is

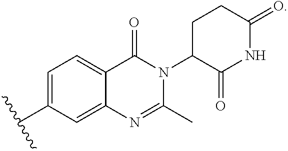 [SMILES: *c1ccc2c(=O)n(C3CCC(=O)NC3=O)c(C)nc2c1]

In some embodiments, LBM is

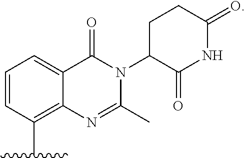 [SMILES: *c1cccc2c(=O)n(C3CCC(=O)NC3=O)c(C)nc12]

In some embodiments, LBM is

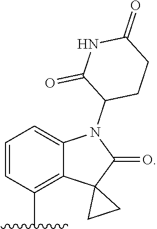 [SMILES: *c1cccc2c1C1(CC1)C(=O)N2C1CCC(=O)NC1=O]

In some embodiments, LBM is

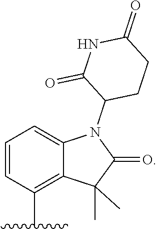 [SMILES: *c1cccc2c1C(C)(C)C(=O)N2C1CCC(=O)NC1=O]

In some embodiments, LBM is selected from those in Table 1A below.

In certain embodiments, the present invention provides methods of degrading STAT6, or a mutant thereof, or methods of treating a STAT6-mediated disease, disorder, or condition, comprising administering a compound of formula I, wherein LBM is an E3 ubiquitin ligase (cereblon) binding moiety thereby forming a compound of formula I-uu:

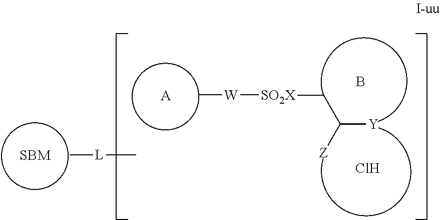

or a pharmaceutically acceptable salt thereof, wherein L and SBM are as defined above and described in embodiments herein, and wherein:
A represents a monocyclic or bicyclic aromatic ring which may be substituted;
B represents a six-membered unsaturated hydrocarbon ring or a six-membered unsaturated heterocycle containing one nitrogen atom as the heteroatom, each of which may be substituted;
C represents a five-membered heterocycle containing one or two nitrogen atoms which may be substituted;
W represents a single bond or a group represented by formula —CH═CH—;
X represents a group represented by formula —N(R¹)— or oxygen;
Y represents carbon or nitrogen;
Z represents a group represented by formula —N(R²)— or nitrogen; and
R¹ and R² may be the same or different from each other and each represent hydrogen or lower alkyl; as described and defined in U.S. Pat. No. 5,721,246, the entirety of each of which is herein incorporated by reference.

In some embodiments, LBM is a IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

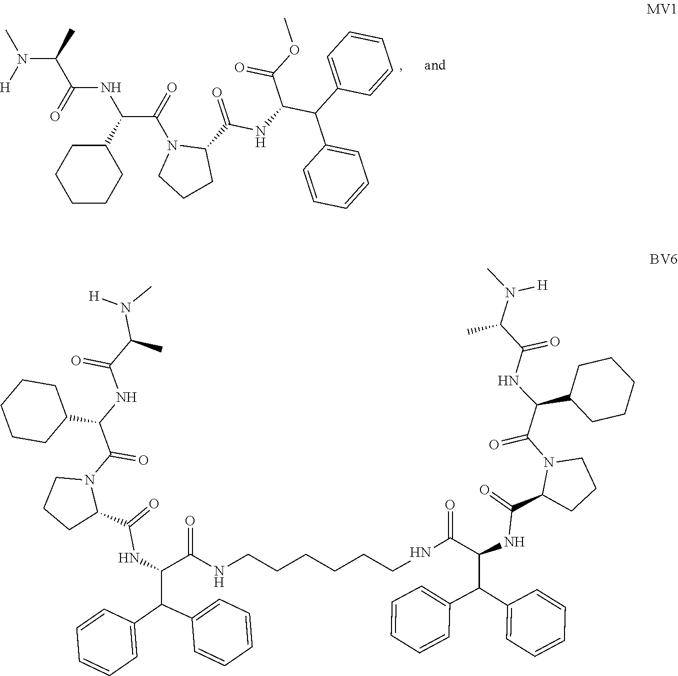 [SMILES: CN[C@@H](C)C(=O)N[C@H](C(=O)N1CCC[C@H]1C(=O)N[C@H](C(=O)OC)C(c1ccccc1)c1ccccc1)C1CCCCC1 ; CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

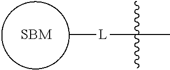

is attached to a modifiable carbon, oxygen, nitrogen or sulfur atom.

In certain embodiments, the present invention provides methods of degrading STAT6, or a mutant thereof, or methods of treating a STAT6-mediated disease, disorder, or condition, comprising administering a compound of Formula I, wherein LBM is a MDM2 (i.e. human double minute 2 or HDM2) E3 ligase binding moiety thereby forming a compound of formula I-aaa-1, I-aaa-2. I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

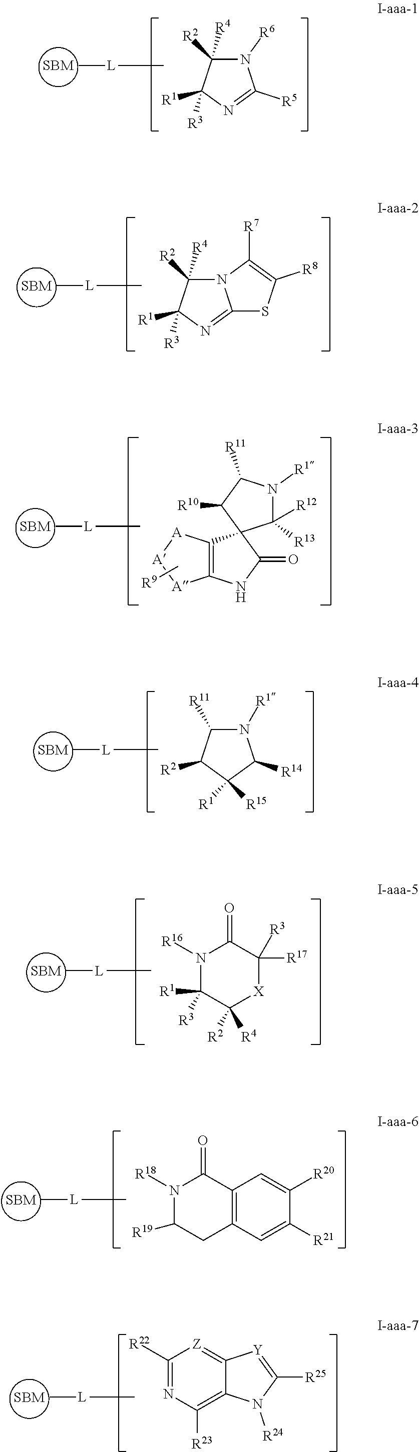

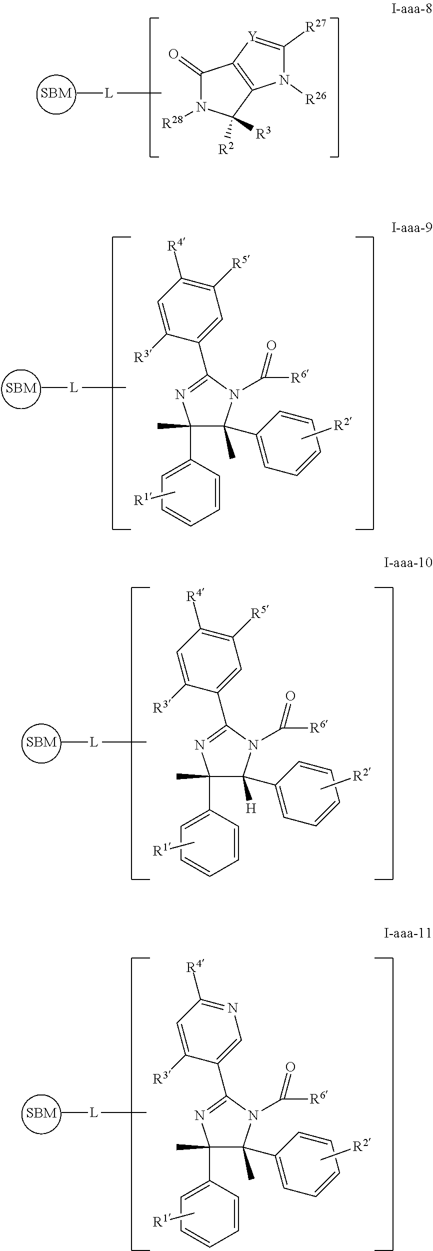

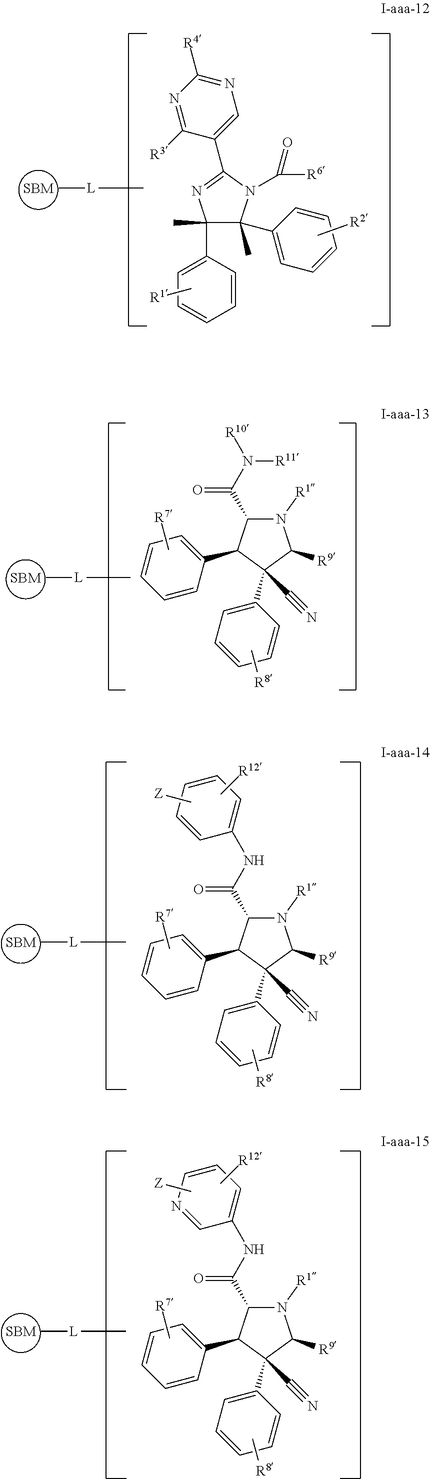

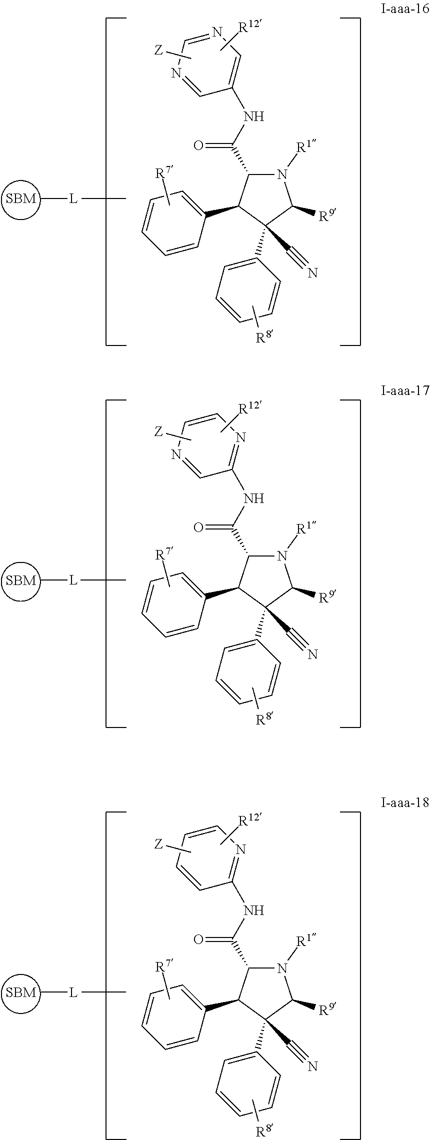

or a pharmaceutically acceptable salt thereof, wherein L and SBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen, and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, his-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A}:
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen. R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides methods of degrading STAT6, or a mutant thereof, or methods of treating a STAT6-mediated disease, disorder, or condition, comprising administering a compound of Formula I, wherein LBM is a MDM2 (i.e. human double minute 2 or HDM2) E3 ligase binding moiety thereby forming a compound of formula I-aaa-19, I-aaa-20, or I-aaa-21 respectively

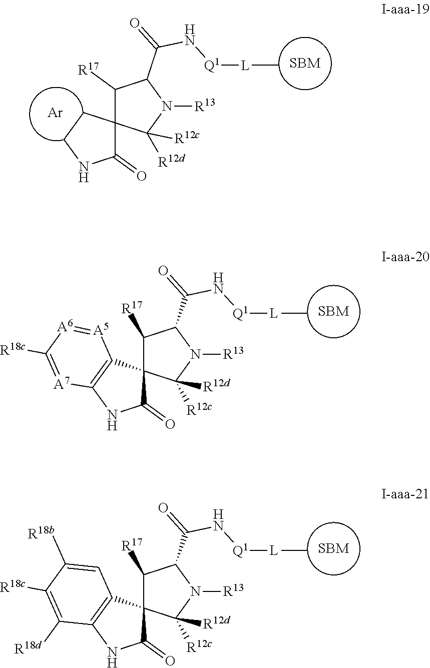

or a pharmaceutically acceptable salt thereof, wherein L and SBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R₁₂ and R₁₃ are each independently selected from hydrogen and R^{A}, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═:
A⁶ is selected from —C(R^{18b})═ and —N═;
A⁷ is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen, and sulfur; and
Q¹ is and optionally substituted bivalent group selected from alkylenyl, phenylenyl, heteroarylenyl, cycloalkylenyl, and heterocyclenyl.

In certain embodiments, the present invention provides methods of degrading STAT6, or a mutant thereof, or methods of treating a STAT6-mediated disease, disorder, or condition, comprising administering a compound of Formula I, wherein LBM is an IAP E3 ubiquitin ligase binding moiety thereby forming a compound of formula I-bbb-1, I-bbb-2, I-bbb-3, or I-bbb-4 respectively:

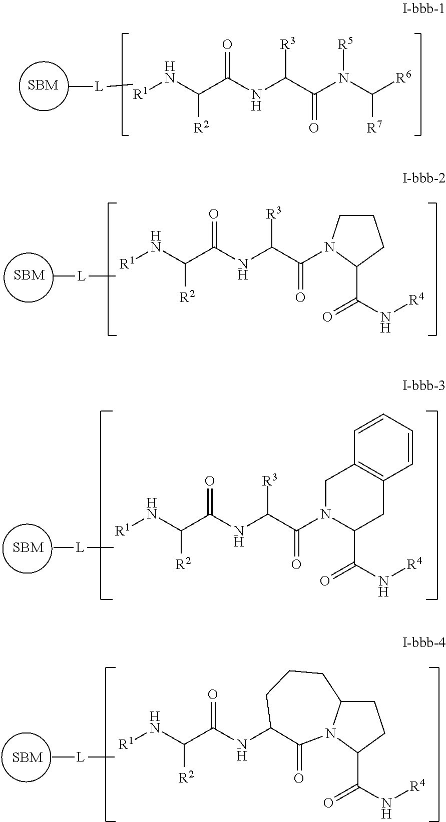

or a pharmaceutically acceptable salt thereof, wherein L and SBM are as defined above and described in embodiments herein, and wherein:
R¹ is selected from the group of H and alkyl;
R² is selected from the group of H and alkyl;
R³ is selected from the group of H, alkyl, cycloalkyl and heterocycloalkyl;
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above;
R⁵ and R⁶ are independently selected from the group of H, alkyl, cycloalkyl, heterocycloalkyl, aryl, heteroaryl or fused rings; and
R⁷ is selected from the group of cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above,
as defined and described in WO 2017/011590 and US 2017/0037004, the entirety of each of which is herein incorporated by reference.

In certain embodiments, the present invention provides methods of degrading STAT6, or a mutant thereof, or methods of treating a STAT6-mediated disease, disorder, or condition, comprising administering a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1, I-ccc-2, or I-ccc-3:

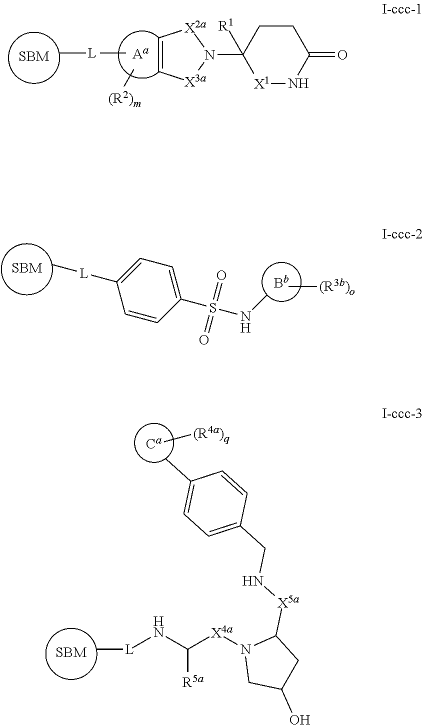

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂—, —C(O)—, —C(S)—,

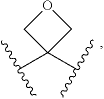 [SMILES: *C1(*)COC1]

-CR₂CR₂, —N═CR—, or —CR═CR—;
each of X^{4a} and X^{5a} is independently a bivalent moiety selected from —CH₂—, —C(O)—, —C(S)—, or

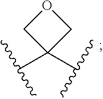 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3a}, and R^{4a} is independently hydrogen, —R⁶, halogen, —CN, —NO₂, —OR, —SR, —NR₂, —S(O)₂R—S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides methods of degrading STAT6, or a mutant thereof, or methods of treating a STAT6-mediated disease, disorder, or condition, comprising administering a compound of Formula I-ccc-1, wherein LBM is an E3 ubiquitin ligase (cereblon) binding moiety thereby forming a compound of formula I-ccc′-1 or I-ccc″-1:

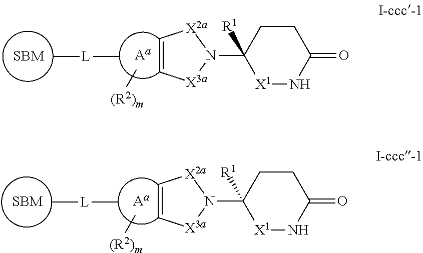

or a pharmaceutically acceptable salt thereof, wherein SBM, L, Ring A^{a}, X¹, X^{2a}, X^{3a}, R¹, R² and m are as described above.

In certain embodiments, the present invention provides methods of degrading STAT6, or a mutant thereof, or methods of treating a STAT6-mediated disease, disorder, or condition, comprising administering a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1′, I-ccc-2′, or I-ccc-3′:

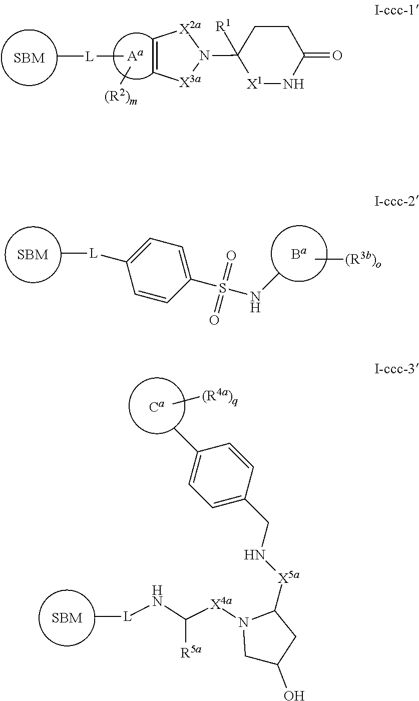

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂—, —C(O)—, —C(S)—,

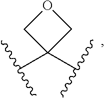 [SMILES: *C1(*)COC1]

-CR₂CR₂, —N═CR—, or —CR═CR—;
each of X^{4a} and X^{5a} is independently a bivalent moiety selected from —CH₂—, —C(O)—, —C(S)—, or

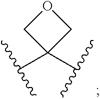 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic:
each of R², R^{b}, and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; or:
   two R² groups of Ring A^{a} are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5- to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 1, 2, 3 or 4: and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides methods of degrading STAT6, or a mutant thereof, or methods of treating a STAT6-mediated disease, disorder, or condition, comprising administering a compound of Formula I-ccc-1′, wherein LBM is an E3 ubiquitin ligase (cereblon) binding moiety thereby forming a compound of formula I-ccc′-1′ or I-ccc″-1′:

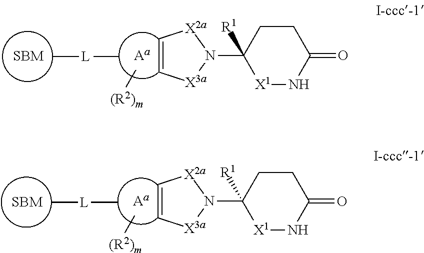

or a pharmaceutically acceptable salt thereof, wherein SBM, L, Ring Aa, X¹, X^{2a}, X^{3a}, R¹, R² and m are as described above.

In some embodiments, LBM is of formulae I-ccc-A, I-ccc-B, or I-ccc-C:

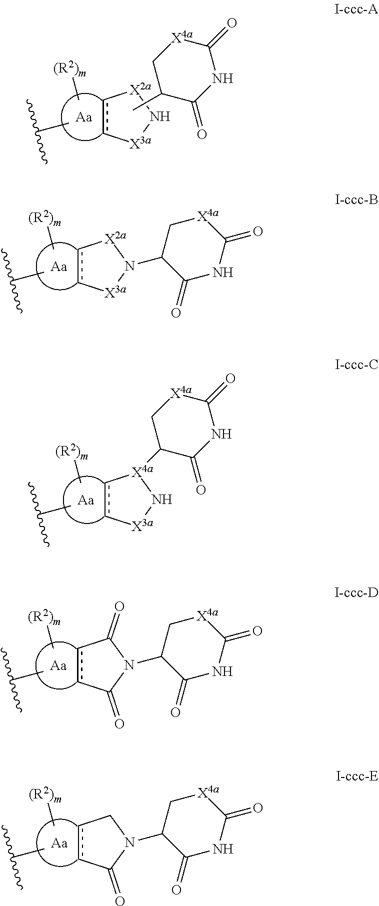

or a pharmaceutically acceptable salt thereof, wherein Ring A^{a}, X^{2a}, X^{3a}, R¹, R² and m are as defined above and described above; and:
X^{4a} is CH, CR, or N.

As defined above and described herein, each of X¹, X^{2a}, and X^{3a} is independently a bivalent moiety selected from a covalent bond, —CH₂—, —C(O)—, —C(S)—,

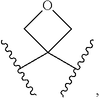 [SMILES: *C1(*)COC1]

—CR₂CR₂, —N═CR—, or —CR═CR—.

In some embodiments, X¹ is a covalent bond, —CH₂—, —C(O)—, —C(S)—,

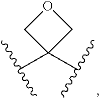 [SMILES: *C1(*)COC1]

—CR₂CR₂, —N═CR—, or —CR═CR—.

In some embodiments, X¹ is selected from those depicted in Table 1A, below.

In some embodiments, X^{2a} is a covalent bond, —CH₂—, —C(O)—, —C(S)—,

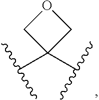 [SMILES: *C1(*)COC1]

CR₂CR₂, —N═CR—, or —CR═CR—. In some embodiments, X^{2a} is —C(O)—.

In some embodiments, X^{2a} is selected from those depicted in Table 1A, below.

In some embodiments, X^{3a} is a covalent bond, —CH₂—, —C(O)—, —C(S)—,

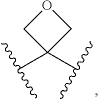 [SMILES: *C1(*)COC1]

—CR₂CR₂, —N═CR—, or —CR═CR—. In some embodiments, X^{3a} is —C(O)—.

In some embodiments, X^{2a} and X^{3a} are —C(O)—. In some embodiments, X^{2a} is —C(O)—: and X^{3a} is —CH₂—.

In some embodiments, X^{3a} is selected from those depicted in Table 1A, below.

As defined above and described herein, X^{4a} is CH, CR, or N. In some embodiments, X^{4a} is CH. In some embodiments, X^{4a} is CR. In some embodiments, X⁴a is N.

As defined above and described herein, each of X⁴ and X¹ is independently a bivalent moiety selected from —CH₂—, —C(O)—, —C(S)—, or

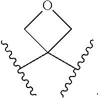 [SMILES: *C1(*)COC1]

In some embodiments, X^{4a} is —CH₂—, —C(O)—, —C(S)—, or

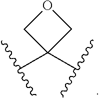 [SMILES: *C1(*)COC1]

In some embodiments, X^{4a} is selected from those depicted in Table 1A, below.

In some embodiments, X^{5a} is —CH₂—, —C(O)—, —C(S)—, or

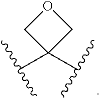 [SMILES: *C1(*)COC1]

In some embodiments, X^{5a} is selected from those depicted in Table 1A, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1A, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN. —NO₂—OR, —SR, —NR₂, —S(O)₂R, —S(O)₂N_{R}—S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, two R² groups of Ring A^{a} are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R² groups of Ring A^{a} are taken together with their intervening atoms to form an optionally substituted ring selected from a 3-10 membered saturated or partially unsaturated carbocyclyl. In some embodiments, two R² groups of Ring A^{a} are taken together with their intervening atoms to form an optionally substituted ring selected from a 5-6 membered saturated or partially unsaturated carbocyclyl.

In some embodiments, two R² groups of Ring A^{a} are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R² groups of Ring A^{a} are taken together with their intervening atoms to form an optionally substituted benzo.

In some embodiments, two R² groups of Ring A^{a} are taken together with their intervening atoms to form an optionally substituted ring selected from a 5-10 membered heteroaryl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in Table 1A, below.

In some embodiments, R^{3b} is hydrogen, —R⁶, halogen, —CN, —NO₂, —OR, —SR, —NR₂, —S(O)₂R, —S(O)₂N_{R}—S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1A, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1A, below.

As defined above and described herein, R^{5a} is hydrogen or C₁₋₆ aliphatic.

In some embodiments, R^{5a} is 1-butyl.

In some embodiments, R^{5a} is selected from those depicted in Table 1A, below.

As defined above and described herein,

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur. In some embodiments, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is an optionally substituted 8-15 membered saturated or partially unsaturated tricyclic heterocyclylenyl having 1-5 heteroatoms independently selected from nitrogen, oxygen, and sulfur.

In some embodiments, R⁶ is a 12-membered saturated or partially unsaturated tricyclic heterocyclylenyl having 1-5 heteroatoms independently selected from nitrogen, oxygen, and sulfur. In some embodiments, R⁶ is a 12-membered saturated or partially unsaturated tricyclic heterocyclylenyl having 1-5 nitrogen heteroatoms. In some embodiments, R⁶ is a 12-membered saturated or partially unsaturated tricyclic heterocyclylenyl having 3 nitrogen heteroatoms.

In some embodiments, R⁶ is a 5,6,5-fused saturated or partially unsaturated tricyclic heterocyclylenyl having 1-5 heteroatoms independently selected from nitrogen, oxygen, and sulfur. In some embodiments, R⁶ is a 5,6,5-fused membered saturated or partially unsaturated tricyclic heterocyclylenyl having 1-5 nitrogen heteroatoms. In some embodiments, R⁶ is a 5,6,5-fused membered saturated or partially unsaturated tricyclic heterocyclylenyl having 3 nitrogen heteroatoms.

In some embodiments, R⁶ is a 5,5,6-fused saturated or partially unsaturated tricyclic heterocyclylenyl having 1-5 heteroatoms independently selected from nitrogen, oxygen, and sulfur. In some embodiments, R⁶ is a 5,5,6-fused membered saturated or partially unsaturated tricyclic heterocyclylenyl having 1-5 nitrogen heteroatoms. In some embodiments, R⁶ is a 5,5,6-fused membered saturated or partially unsaturated tricyclic heterocyclylenyl having 3 nitrogen heteroatoms.

In some embodiments, R⁶ is a 13-membered saturated or partially unsaturated tricyclic heterocyclylenyl having 1-5 heteroatoms independently selected from nitrogen, oxygen, and sulfur. In some embodiments, R⁶ is a 13-membered saturated or partially unsaturated tricyclic heterocyclylenyl having 1-5 nitrogen heteroatoms. In some embodiments, R⁶ is a 13-membered saturated or partially unsaturated tricyclic heterocyclylenyl having 3 nitrogen heteroatoms.

In some embodiments, R⁶ is a 5,6,6-fused saturated or partially unsaturated tricyclic heterocyclylenyl having 1-5 heteroatoms independently selected from nitrogen, oxygen, and sulfur. In some embodiments, R⁶ is a 5,6,6-fused membered saturated or partially unsaturated tricyclic heterocyclylenyl having 1-5 nitrogen heteroatoms. In some embodiments, R⁶ is a 5,6,6-fused membered saturated or partially unsaturated tricyclic heterocyclylenyl having 3 nitrogen heteroatoms.

In some embodiments, R⁶ is a 6,5,6-fused saturated or partially unsaturated tricyclic heterocyclylenyl having 1-5 heteroatoms independently selected from nitrogen, oxygen, and sulfur. In some embodiments, R⁶ is a 6,5,6-fused membered saturated or partially unsaturated tricyclic heterocyclylenyl having 1-5 nitrogen heteroatoms. In some embodiments, R⁶ is a 6,5,6-fused membered saturated or partially unsaturated tricyclic heterocyclylenyl having 3 nitrogen heteroatoms.

In some embodiments, R⁶ is a 14-membered saturated or partially unsaturated tricyclic heterocyclylenyl having 1-5 heteroatoms independently selected from nitrogen, oxygen, and sulfur.

In some embodiments, R⁶ is or an 10-15 membered tricyclic heteroarylenyl having 1-5 heteroatoms independently selected from nitrogen, oxygen, and sulfur.

In some embodiments, R⁶ is a 12-membered saturated or partially unsaturated tricyclic heteroarylenyl having 1-5 heteroatoms independently selected from nitrogen, oxygen, and sulfur. In some embodiments, R⁶ is a 12-membered saturated or partially unsaturated tricyclic heteroarylenyl having 1-5 nitrogen heteroatoms. In some embodiments, R⁶ is a 12-membered saturated or partially unsaturated tricyclic heteroarylenyl having 3 nitrogen heteroatoms.

In some embodiments, R⁶ is a 5,6,5-fused saturated or partially unsaturated tricyclic heteroarylenyl having 1-5 heteroatoms independently selected from nitrogen, oxygen, and sulfur. In some embodiments, R⁶ is a 5,6,5-fused membered saturated or partially unsaturated tricyclic heteroarylenyl having 1-5 nitrogen heteroatoms. In some embodiments, R⁶ is a 5,6,5-fused membered saturated or partially unsaturated tricyclic heteroarylenyl having 3 nitrogen heteroatoms.

In some embodiments, R⁶ is a 5,5,6-fused saturated or partially unsaturated tricyclic heteroarylenyl having 1-5 heteroatoms independently selected from nitrogen, oxygen, and sulfur. In some embodiments, R⁶ is a 5,5,6-fused membered saturated or partially unsaturated tricyclic heteroarylenyl having 1-5 nitrogen heteroatoms. In some embodiments, R⁶ is a 5,5,6-fused membered saturated or partially unsaturated tricyclic heteroarylenyl having 3 nitrogen heteroatoms.

In some embodiments, R⁶ is a 13-membered saturated or partially unsaturated tricyclic heteroarylenyl having 1-5 heteroatoms independently selected from nitrogen, oxygen, and sulfur. In some embodiments, R⁶ is a 13-membered saturated or partially unsaturated tricyclic heteroarylenyl having 1-5 nitrogen heteroatoms. In some embodiments, R⁶ is a 13-membered saturated or partially unsaturated tricyclic heteroarylenyl having 3 nitrogen heteroatoms.

In some embodiments, R⁶ is a 5,6,6-fused saturated or partially unsaturated tricyclic heteroarylenyl having 1-5 heteroatoms independently selected from nitrogen, oxygen, and sulfur. In some embodiments, R⁶ is a 5,6,6-fused membered saturated or partially unsaturated tricyclic heteroarylenyl having 1-5 nitrogen heteroatoms. In some embodiments, R⁶ is a 5,6,6-fused membered saturated or partially unsaturated tricyclic heteroarylenyl having 3 nitrogen heteroatoms.

In some embodiments, R⁶ is a 6,5,6-fused saturated or partially unsaturated tricyclic heteroarylenyl having 1-5 heteroatoms independently selected from nitrogen, oxygen, and sulfur. In some embodiments, R⁶ is a 6,5,6-fused membered saturated or partially unsaturated tricyclic heteroarylenyl having 1-5 nitrogen heteroatoms. In some embodiments, R⁶ is a 6,5,6-fused membered saturated or partially unsaturated tricyclic heteroarylenyl having 3 nitrogen heteroatoms.

In some embodiments, R⁶ is selected from those depicted in Table 1A, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen or sulfur. In some embodiments Ring A^{a} is a fused 5-membered heteroaryl with 1-3 heteroatoms independently selected from nitrogen, oxygen or sulfur.

In some embodiments, Ring A^{a} is a fused phenyl.

In some embodiments, Ring A^{a} is selected from those depicted in Table 1A, below.

As defined above and described herein, Ring Ba is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B^{a} is a 6-membered aryl containing 0-2 nitrogen atoms. In some embodiments, Ring Ba is a 8-10 membered bicyclic heteroaryl having 1-5 heteroatoms independently selected from nitrogen, oxygen, or sulfur.

In some embodiments, Ring B^{a} is

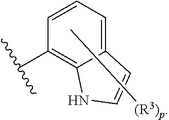

In some embodiments, Ring B^{a} is selected from those depicted in Table 1A, below.

As defined above and described herein. Ring C^{a} is selected from 6-membered aryl containing 0-2 nitrogen atoms or a 5-membered heteroaryl with 1-3 heteroatoms independently selected from nitrogen, oxygen or sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen or sulfur.

In some embodiments, Ring C^{a} is

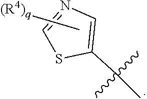

In some embodiments, Ring Ca is selected from those depicted Table 1A, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1A, below.

In some embodiments, o is selected from those depicted in Table 1A, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1A, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1A, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1A, below.

In certain embodiments, the present invention provides methods of degrading STAT6, or a mutant thereof, or methods of treating a STAT6-mediated disease, disorder, or condition, comprising administering a compound of formula I, wherein LBM is a VHL E3 ubiquitin ligase binding moiety, thereby forming a compound of formula I-ddd:

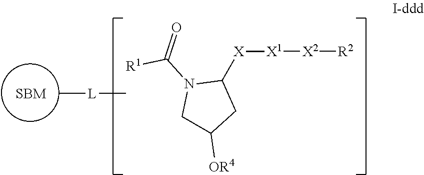

or a pharmaceutically acceptable salt thereof, wherein L and SBM is as defined above and described in embodiments herein, and wherein:
X is —C(O)—, —C(O)NR—, —SO₂—, —SO₂NR—, or an optionally substituted 5-membered heterocyclic ring:
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;

R² is hydrogen, halogen, —CN,

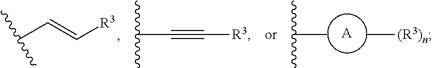

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR. —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

In certain embodiments, the present invention provides methods of degrading STAT6, or a mutant thereof, or methods of treating a STAT6-mediated disease, disorder, or condition, comprising administering a compound of formula I, wherein LBM is an IAP binding moiety thereby forming a compound of formula I-fff:

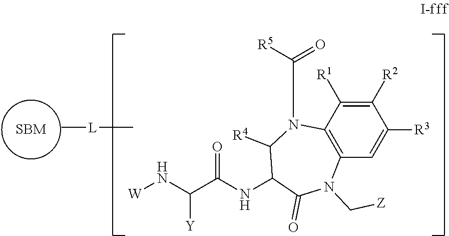

or a pharmaceutically acceptable salt thereof, wherein L and SBM are as defined above and described in embodiments herein, and wherein:
W is selected from H and lower alkyl that optionally may be substituted with 1-3 deuterium atoms;
Y is lower alkyl that optionally may be substituted with OR⁶;
R¹, R² and R³ are the same or different and each is independently selected from H and cyano;
R⁴ is lower alkyl;
R⁵ is selected from the group a) lower alkyl that optionally may be substituted with SO₂R⁶ and OR⁶, b) heterocyclyl, and c) aryl that optionally may be substituted with C(O)R⁷, halo and cyano;
Z is selected from the group a) aryl that optionally may be substituted with lower alkyl. OR, halogen and aryl that optionally may be substituted with halogen, b) heteroaryl that optionally may be substituted with lower alkyl, cycloalkyl, OR⁶, halogen, oxo and aryl that optionally may substituted with cyano, and c) aryl fused with heterocyclyl, wherein the aryl optionally may be substituted with OR⁶ and halogen, and the heterocyclyl optionally may be substituted with oxo, and d) heterocyclyl;
R⁶ is selected from H and lower alkyl that optionally may be substituted with halogen and deuterium; and
R⁷ is lower alkyl,
as described and defined in WO 2014/044622, US 2015/0225449, WO 2015/071393, and US 2016/0272596, the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a MDM2 binding moiety thereby forming a compound of formula I-ggg:

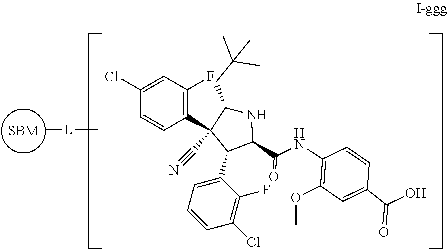

or a pharmaceutically acceptable salt thereof, wherein L and SBM are as defined above and described in embodiments herein, as described and defined in Hines, J. et al., Cancer Res. (DOI: 10.1158/0008-5472.CAN-18-2918), the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a DCAF16 binding moiety thereby forming a compound of formula I-hhh:

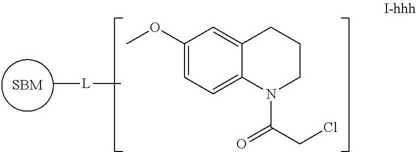

or a pharmaceutically acceptable salt thereof, wherein L and SBM are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a RNF114 binding moiety thereby forming a compound of formula I-iii:

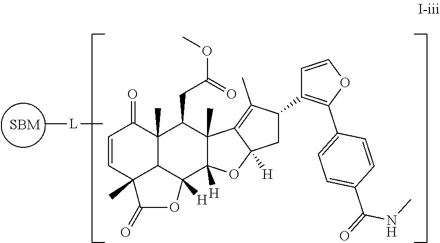

or a pharmaceutically acceptable salt thereof, wherein L and SBM are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a RNF4 binding moiety thereby forming a compound of formula I-jjj:

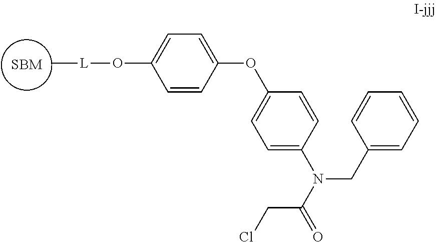

or a pharmaceutically acceptable salt thereof, wherein L and SBM are as defined above and described in embodiments herein, as described and defined in Ward, C. C., et al., bioRxiv (doi: https://doi.org/10.1101/143925), the entirety of each of which is herein incorporated by reference.

In some embodiments, LBM is

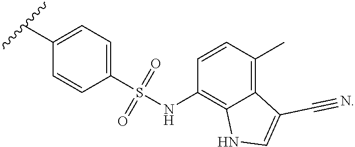 [SMILES: *c1ccc(S(=O)(=O)Nc2ccc(C)c3c(C#N)c[nH]c23)cc1]

In some embodiments, LBM is

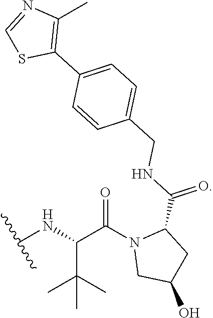 [SMILES: *N[C@H](C(=O)N1C[C@H](O)C[C@H]1C(=O)NCc1ccc(-c2scnc2C)cc1)C(C)(C)C]

In some embodiments, LBM is

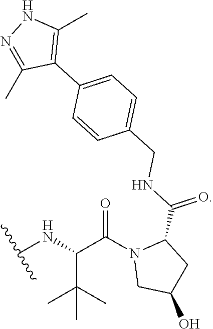 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

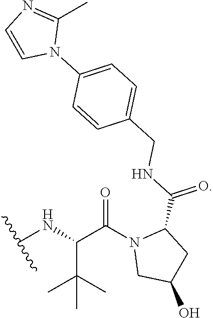 [SMILES: *N[C@H](C(=O)N1C[C@H](O)C[C@H]1C(=O)NCc1ccc(-n2ccnc2C)cc1)C(C)(C)C]

In some embodiments, LBM is

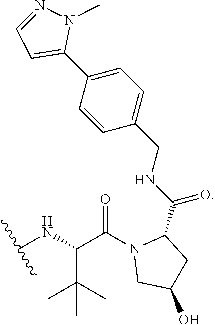 [SMILES: *N[C@H](C(=O)N1C[C@H](O)C[C@H]1C(=O)NCc1ccc(-c2ccnn2C)cc1)C(C)(C)C]

In some embodiments, LBM is

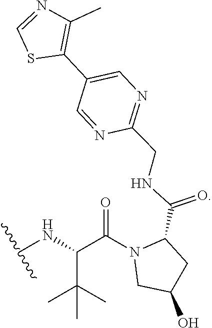 [SMILES: *N[C@H](C(=O)N1C[C@H](O)C[C@H]1C(=O)NCc1ncc(-c2scnc2C)cn1)C(C)(C)C]

In some embodiments, LBM is

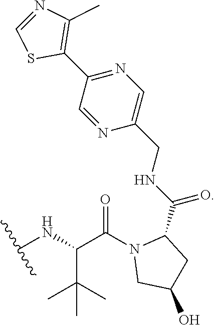 [SMILES: *N[C@H](C(=O)N1C[C@H](O)C[C@H]1C(=O)NCc1cnc(-c2scnc2C)cn1)C(C)(C)C]

In some embodiments, LBM is

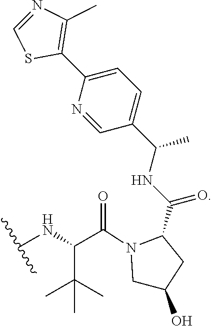 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

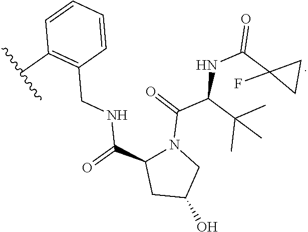 [SMILES: *c1ccccc1CNC(=O)[C@@H]1C[C@@H](O)CN1C(=O)[C@@H](NC(=O)C1(F)CC1)C(C)(C)C]

In some embodiments, LBM is

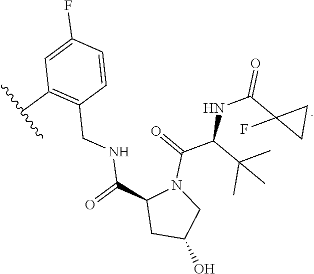 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

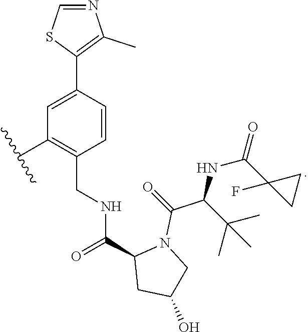 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

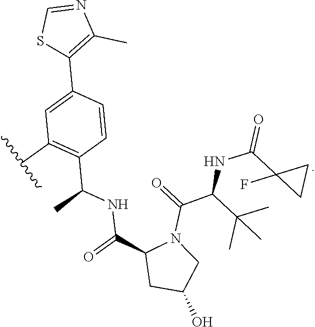 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C]

In some embodiments LBM is

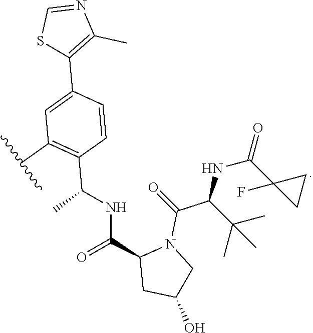 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

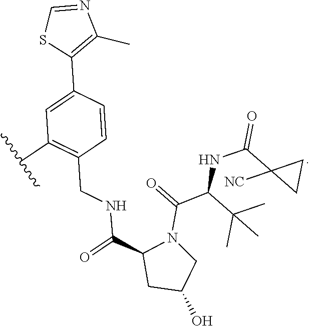 [SMILES: *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, LBM is

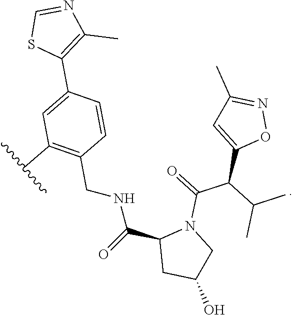 [SMILES: *c1cc(-c2scnc2C)ccc1CNC(=O)[C@@H]1C[C@@H](O)CN1C(=O)[C@@H](c1cc(C)no1)C(C)C]

In some embodiments, LBM is

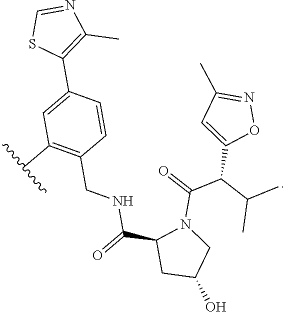 [SMILES: *c1cc(-c2scnc2C)ccc1CNC(=O)[C@@H]1C[C@@H](O)CN1C(=O)[C@H](c1cc(C)no1)C(C)C]

In some embodiments, LBM is

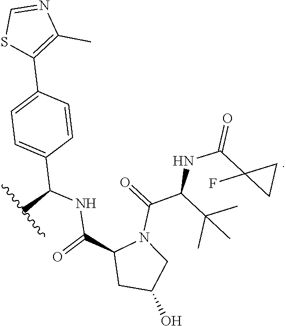 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

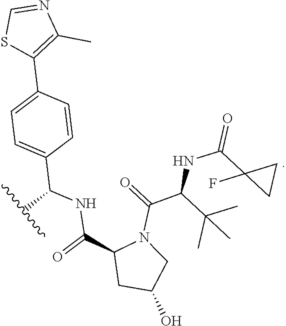 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

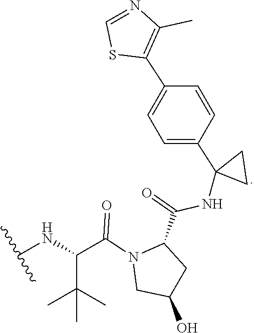 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

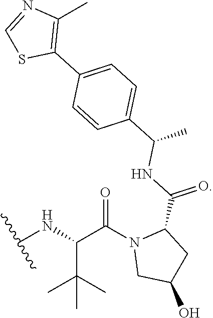 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

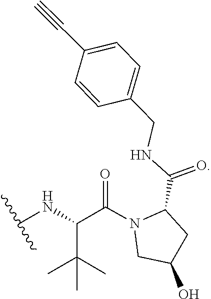 [SMILES: *N[C@H](C(=O)N1C[C@H](O)C[C@H]1C(=O)NCc1ccc(C#C)cc1)C(C)(C)C]

In some embodiments, LBM is

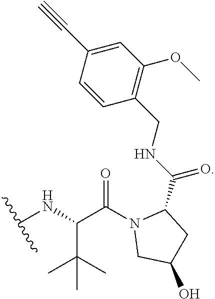 [SMILES: *N[C@H](C(=O)N1C[C@H](O)C[C@H]1C(=O)NCc1ccc(C#C)cc1OC)C(C)(C)C]

In some embodiments, LBM is

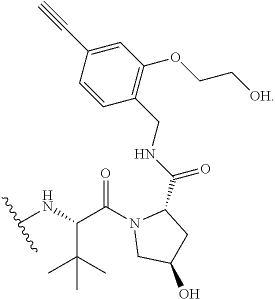 [SMILES: *N[C@H](C(=O)N1C[C@H](O)C[C@H]1C(=O)NCc1ccc(C#C)cc1OCCO)C(C)(C)C]

In some embodiments, LBM is

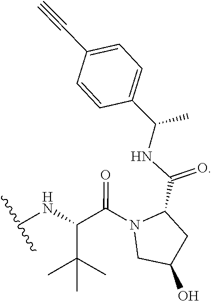 [SMILES: *N[C@H](C(=O)N1C[C@H](O)C[C@H]1C(=O)N[C@@H](C)c1ccc(C#C)cc1)C(C)(C)C]

In some embodiments, LBM is

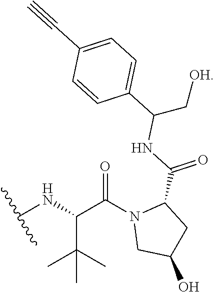 [SMILES: *N[C@H](C(=O)N1C[C@H](O)C[C@H]1C(=O)NC(CO)c1ccc(C#C)cc1)C(C)(C)C]

In some embodiments, LBM is

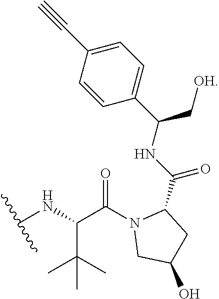 [SMILES: *N[C@H](C(=O)N1C[C@H](O)C[C@H]1C(=O)N[C@H](CO)c1ccc(C#C)cc1)C(C)(C)C]

In some embodiments, LBM is

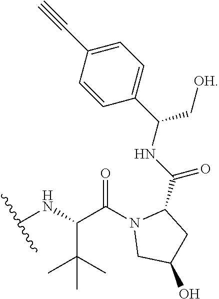 [SMILES: *N[C@H](C(=O)N1C[C@H](O)C[C@H]1C(=O)N[C@@H](CO)c1ccc(C#C)cc1)C(C)(C)C]

In some embodiments, LBM is

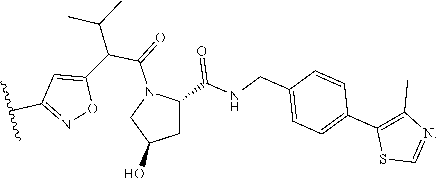 [SMILES: *c1cc(C(C(=O)N2C[C@H](O)C[C@H]2C(=O)NCc2ccc(-c3scnc3C)cc2)C(C)C)on1]

In some embodiments, LBM is

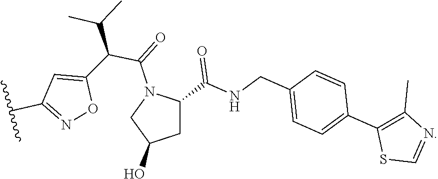 [SMILES: *c1cc([C@H](C(=O)N2C[C@H](O)C[C@H]2C(=O)NCc2ccc(-c3scnc3C)cc2)C(C)C)on1]

In some embodiments, LBM is

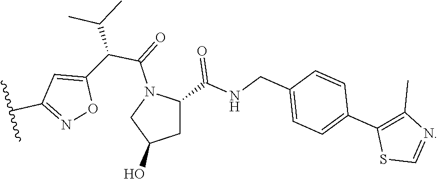 [SMILES: *c1cc([C@@H](C(=O)N2C[C@H](O)C[C@H]2C(=O)NCc2ccc(-c3scnc3C)cc2)C(C)C)on1]

In some embodiments, LBM is

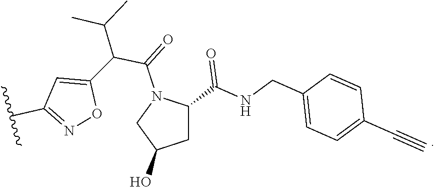 [SMILES: *c1cc(C(C(=O)N2C[C@H](O)C[C@H]2C(=O)NCc2ccc(C#C)cc2)C(C)C)on1]

In some embodiments, LBM is

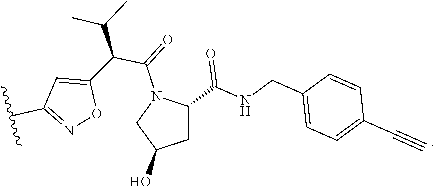 [SMILES: *c1cc([C@H](C(=O)N2C[C@H](O)C[C@H]2C(=O)NCc2ccc(C#C)cc2)C(C)C)on1]

In some embodiments, LBM is

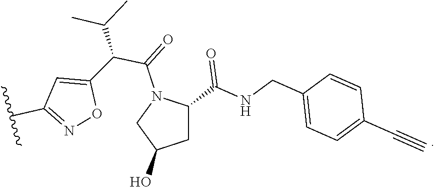 [SMILES: *c1cc([C@@H](C(=O)N2C[C@H](O)C[C@H]2C(=O)NCc2ccc(C#C)cc2)C(C)C)on1]

In some embodiments, LBM is

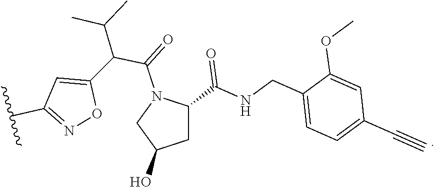 [SMILES: *c1cc(C(C(=O)N2C[C@H](O)C[C@H]2C(=O)NCc2ccc(C#C)cc2OC)C(C)C)on1]

In some embodiments, LBM is

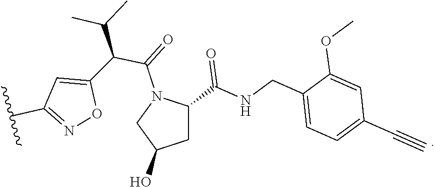 [SMILES: *c1cc([C@H](C(=O)N2C[C@H](O)C[C@H]2C(=O)NCc2ccc(C#C)cc2OC)C(C)C)on1]

In some embodiments, LBM is

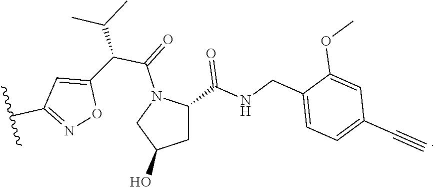 [SMILES: *c1cc([C@@H](C(=O)N2C[C@H](O)C[C@H]2C(=O)NCc2ccc(C#C)cc2OC)C(C)C)on1]

In some embodiments, LBM is

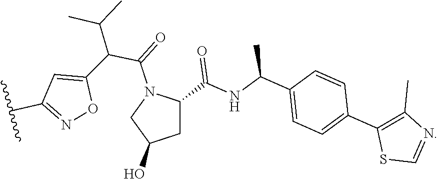 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

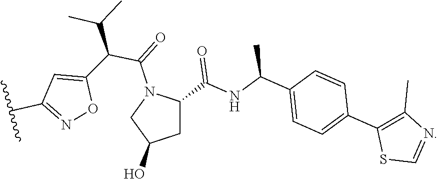 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

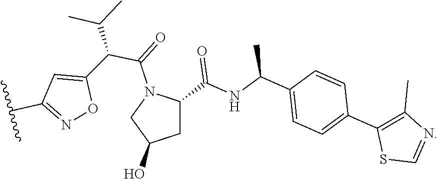 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

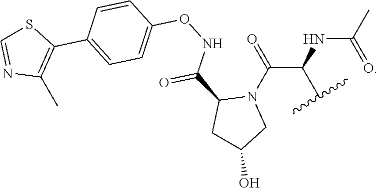 [SMILES: *[C@H](NC(C)=O)C(=O)N1C[C@H](O)C[C@H]1C(=O)NOc1ccc(-c2scnc2C)cc1]

In some embodiments, LBM is

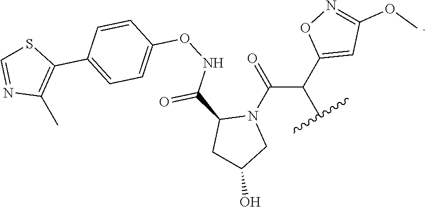 [SMILES: *C(C(=O)N1C[C@H](O)C[C@H]1C(=O)NOc1ccc(-c2scnc2C)cc1)c1cc(OC)no1]

In some embodiments, LBM is

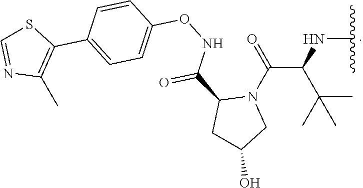 [SMILES: *N[C@H](C(=O)N1C[C@H](O)C[C@H]1C(=O)NOc1ccc(-c2scnc2C)cc1)C(C)(C)C]

In some embodiments, LBM is

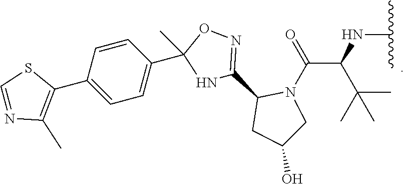 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

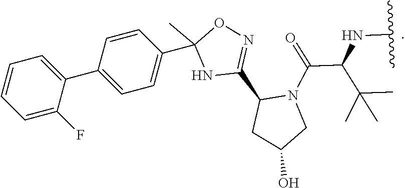 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

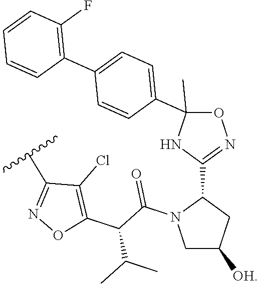 [SMILES: *c1noc([C@H](C(=O)N2C[C@H](O)C[C@H]2C2=NOC(C)(c3ccc(-c4ccccc4F)cc3)N2)C(C)C)c1Cl]

In some embodiments, LBM is

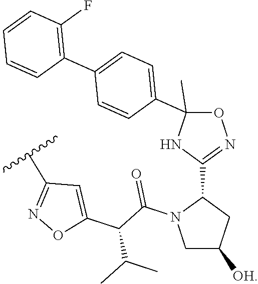 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

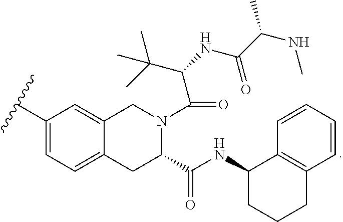 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

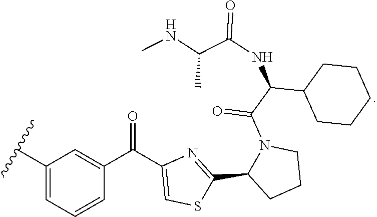 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

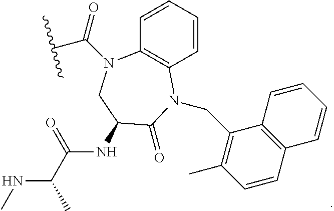 [SMILES: *C(=O)N1C[C@H](NC(=O)[C@H](C)NC)C(=O)N(Cc2c(C)ccc3ccccc23)c2ccccc21]

In some embodiments, LBM is

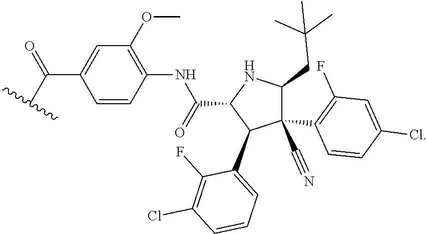 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

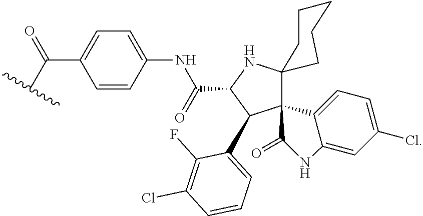 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

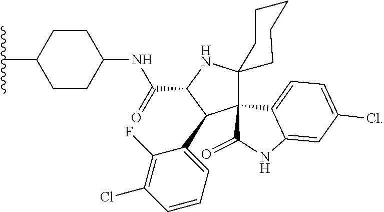 [SMILES: *C1CCC(NC(=O)[C@@H]2NC3(CCCCC3)[C@@]3(C(=O)Nc4cc(Cl)ccc43)[C@H]2c2cccc(Cl)c2F)CC1]

In some embodiments, LBM is

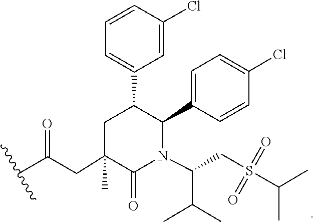 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

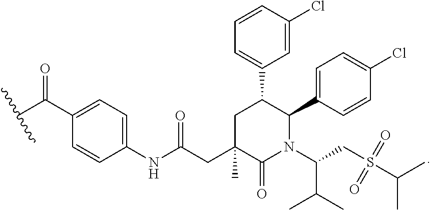 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

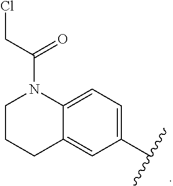 [SMILES: *c1ccc2c(c1)CCCN2C(=O)CCl]

In some embodiments, LBM is

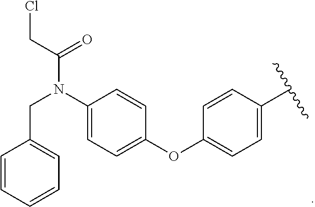 [SMILES: *c1ccc(Oc2ccc(N(Cc3ccccc3)C(=O)CCl)cc2)cc1]

In some embodiments, LBM is

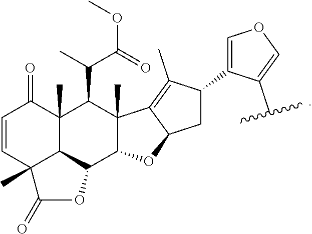 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

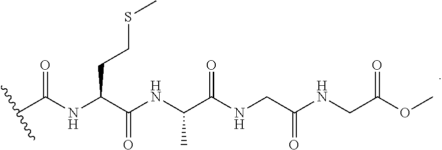 [SMILES: *C(=O)N[C@@H](CCSC)C(=O)N[C@@H](C)C(=O)NCC(=O)NCC(=O)OC]

In some embodiments, LBM is

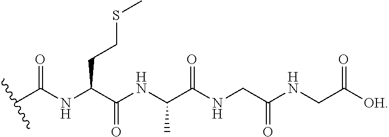 [SMILES: *C(=O)N[C@@H](CCSC)C(=O)N[C@@H](C)C(=O)NCC(=O)NCC(=O)O]

In some embodiments, LBM is

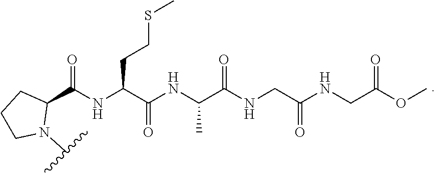 [SMILES: *N1CCC[C@H]1C(=O)N[C@@H](CCSC)C(=O)N[C@@H](C)C(=O)NCC(=O)NCC(=O)OC]

In some embodiments, LBM is

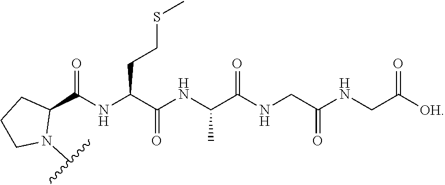 [SMILES: *N1CCC[C@H]1C(=O)N[C@@H](CCSC)C(=O)N[C@@H](C)C(=O)NCC(=O)NCC(=O)O]

In some embodiments, LBM is

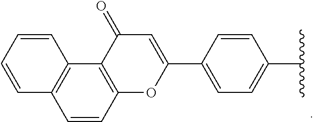 [SMILES: *c1ccc(-c2cc(=O)c3c(ccc4ccccc43)o2)cc1]

In some embodiments, LBM is

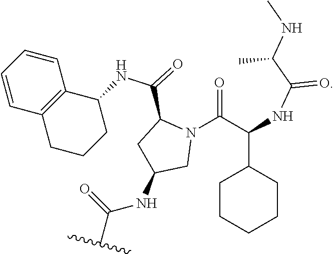 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

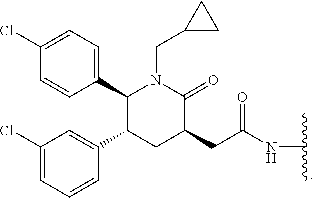 [SMILES: *NC(=O)C[C@H]1C[C@H](c2cccc(Cl)c2)[C@@H](c2ccc(Cl)cc2)N(CC2CC2)C1=O]

In some embodiments, LBM is

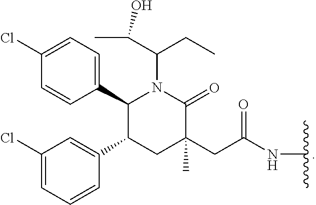 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

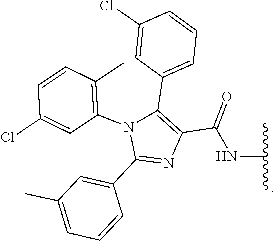 [SMILES: *NC(=O)c1nc(-c2cccc(C)c2)n(-c2cc(Cl)ccc2C)c1-c1cccc(Cl)c1]

In some embodiments, LBM is

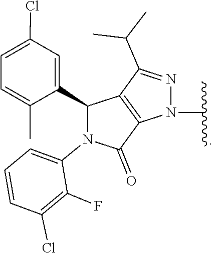 [SMILES: *n1nc(C(C)C)c2c1C(=O)N(c1cccc(Cl)c1F)[C@H]2c1cc(Cl)ccc1C]

In some embodiments, LBM is

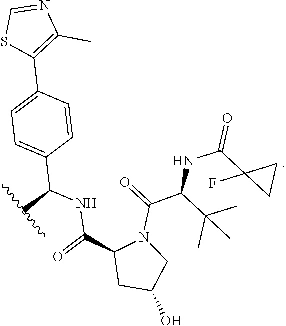 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In certain embodiments, the present invention provides methods of degrading STAT6, or a mutant thereof, or methods of treating a STAT6-mediated disease, disorder, or condition, comprising administering a compound of formula I, wherein LBM is a CRBN E3 ubiquitin ligase binding moiety thereby forming a compound of formula I-qqq:

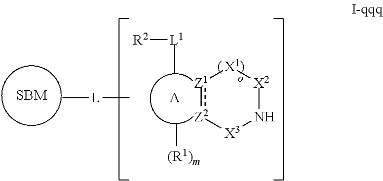

or a pharmaceutically acceptable salt thereof, wherein L and SBM are as defined above and described in embodiments herein, wherein:
each X¹ is independently —CH₂—, —O—, —NR—, —CF₂—,

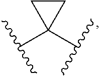 [SMILES: *C1(*)CC1]

-C(O)—, —C(S)—, or

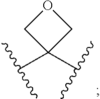 [SMILES: *C1(*)COC1]

X² and X³ are independently —CH₂—, —C(O)—, —C(S)— or

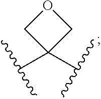 [SMILES: *C1(*)COC1]

Z¹ and Z² are independently a carbon atom or a nitrogen atom;
   Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—. —C(O)—, —C(S)—, —CR₂—, —CRF—, —CF₂—, —NR—, or —S(O)₂—:
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR—N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃: or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

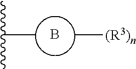

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups:
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃:
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
o is 0, 1, or 2.

As defined above and described herein each X¹ is independently a covalent bond, —CH₂—, —O—, —NR—, —CF₂—,

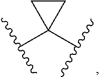 [SMILES: *C1(*)CC1]

### —C(O)—, —C(S)—, or

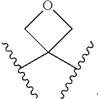 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

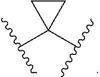 [SMILES: *C1(*)CC1]

In some embodiments, X¹ is —C(O)—. In some embodiments, X¹ is —C(S)—. In some embodiments, X¹ is

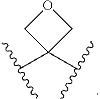 [SMILES: *C1(*)COC1]

In certain embodiments, X¹ is selected from those shown in the compounds of Table LA.

As defined above and described herein, X² and X³ are independently —CH₂—, —C(O)—, —C(S)—, or

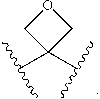 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

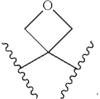 [SMILES: *C1(*)COC1]

In certain embodiments, X² and X³ are independently selected from those shown in the compounds of Table 1A.

As defined above and described herein, X⁴ is a covalent bond, —CH₂—, —CR₂—, —O—, —NR—, —CF₂—,

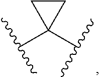 [SMILES: *C1(*)CC1]

### —C(O)—, —C(S)—, or

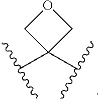 [SMILES: *C1(*)COC1]

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1A.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In certain embodiments, Ring A is selected from those shown in the compounds of Table 1A.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1A.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)z, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In certain embodiments, each R¹ is independently selected from those shown in the compounds of Table 1A.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R¹ is selected from

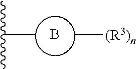

or hydrogen.

In some embodiment R² is

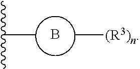

In some embodiments, R² is hydrogen.

In certain embodiments, R² is selected from those shown in the compounds of Table 1A.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table LA.

As defined above and described herein, each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R², —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1A.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1A.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table LA.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1A.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1A.

As defined above and described herein, o is 0, 1, or 2.

In some embodiments, n is 0. In some embodiments, n is 1. In some embodiments, m is 2.

In certain embodiments, o is selected from those shown in the compounds of Table 1A.

In some embodiments, the present invention provides methods of degrading STAT6, or a mutant thereof, or methods of treating a STAT6-mediated disease, disorder, or condition, comprising administering a compound of formula I-qqq-A:

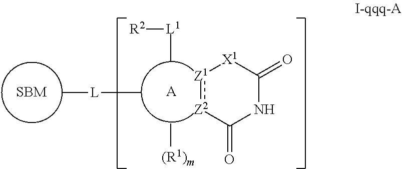

or a pharmaceutically acceptable salt thereof, wherein each of SBM, L, L¹, R¹, R², m, Z¹, Z², and X¹ is as defined above and described in embodiments herein, both singly and in combination.

In some embodiments, the present invention provides methods of degrading STAT6, or a mutant thereof, or methods of treating a STAT6-mediated disease, disorder, or condition, comprising administering a compound of formula I-qqq-B:

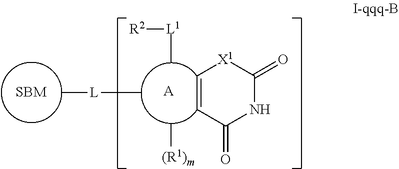

or a pharmaceutically acceptable salt thereof, wherein each of SBM, L, L¹, R¹, R², m, and X¹ is as defined above and described in embodiments herein, both singly and in combination.

In some embodiments, the present invention provides methods of degrading STAT6, or a mutant thereof, or methods of treating a STAT6-mediated disease, disorder, or condition, comprising administering a compound of formula I-qqq, wherein Ring A is benzo, o is 1, X¹ is —CH₂—, X² and X³ are —C(O)—, and Z¹ and Z² are carbon atoms as shown, to provide a compound of formula I-qqq-1:

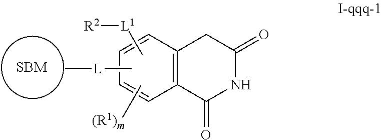

or a pharmaceutically acceptable salt thereof, wherein each of SBM, L, L¹, R¹, R², and m is as defined above and described in embodiments herein, both singly and in combination.

In some embodiments, the present invention provides methods of degrading STAT6, or a mutant thereof, or methods of treating a STAT6-mediated disease, disorder, or condition, comprising administering a compound of formula I-qqq, wherein Ring A is benzo, o is 1, X¹, X² and X³ are —C(O)—, and Z¹ and Z² are carbon atoms as shown, to provide a compound of formula I-qqq-12:

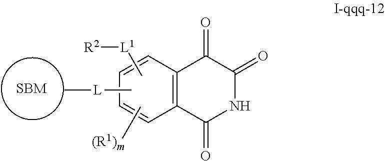

or a pharmaceutically acceptable salt thereof, wherein each of SBM, L, L¹, R¹, R², and m is as defined above and described in embodiments herein, both singly and in combination.

In some embodiments, LBM is

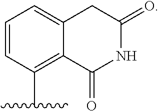 [SMILES: *c1cccc2c1C(=O)NC(=O)C2]

In some embodiments, LBM is

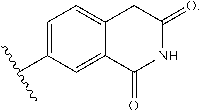 [SMILES: *c1ccc2c(c1)C(=O)NC(=O)C2]

In some embodiments, LBM is

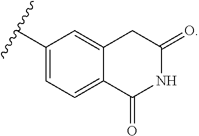 [SMILES: *c1ccc2c(c1)CC(=O)NC2=O]

In some embodiments, LBM is

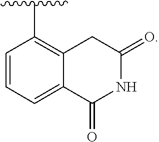 [SMILES: *c1cccc2c1CC(=O)NC2=O]

In some embodiments, LBM is

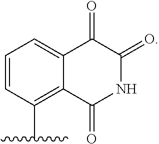 [SMILES: *c1cccc2c1C(=O)NC(=O)C2=O]

In some embodiments, LBM is

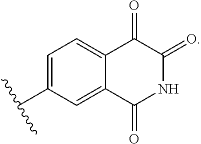 [SMILES: *c1ccc2c(c1)C(=O)NC(=O)C2=O]

In some embodiments, LBM is

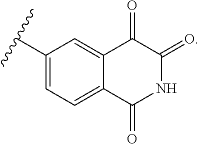 [SMILES: *c1ccc2c(c1)C(=O)C(=O)NC2=O]

In some embodiments, LBM is

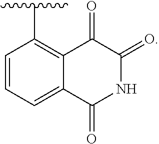 [SMILES: *c1cccc2c1C(=O)C(=O)NC2=O]

In some embodiments, LBM is selected from those in Table 1A, below.

In certain embodiments, the present invention provides methods of degrading STAT6, or a mutant thereof, or methods of treating a STAT6-mediated disease, disorder, or condition, comprising administering a compound of formula I, wherein LBM is a RPN13 binding moiety thereby forming a compound of formula I-rrr:

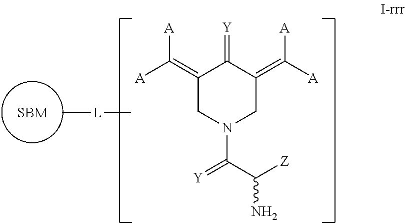

or a pharmaceutically acceptable salt thereof, wherein L and SBM are as defined above and described in embodiments herein, and wherein:
in each pair of A's, one A is hydrogen, and the other A is one of
(i) phenyl, optionally substituted with 1-5 substituents selected from the group consisting of R¹, OR¹, NR¹R², S(O)_{q}R¹, SO₂R¹R², NR¹SO₂R², C(O)R¹, C(O)OR¹, C(O)NR¹R², NR¹C(O)R². NR¹C(O)OR², CF₃, and OCF₃:
(ii) naphthyl, optionally substituted with 1-5 substituents selected from the group consisting of R¹, OR¹, NR¹R², S(O)_{q}R¹, SO₂R¹R², NR¹SO₂R², C(O)R¹, C(O)OR¹, C(O)NR¹R₂, NR¹C(O)R², NR¹C(O)OR₂, CF₃, and OCF₃;
(iii) a 5 or 6 membered monocyclic heteroaryl group, having 1-3 heteroatoms selected from the group consisting of O, N, and S, optionally substituted with 1-3 substituents selected from the group consisting of R¹, OR¹, NR₁R², S(O)^{q}R¹, SO₂R¹R², NR₁SO₂R², C(O)R¹, C(O)OR¹, C(O)NR¹R², NR¹C(O)R², NR¹C(O)OR², CF₃, and OCF₃; and
(iv) an 8 to 10 membered bicyclic heteroalkyl group containing 1-3 heteroatoms selected from the group consisting of O, N, and S; and the second ring is fused to the first ring using 3 to 4 carbon atoms, and the bicyclic hetero aryl group is optionally substituted with 1-3 substituents selected from the group consisting of R¹, OR¹, NR¹R², SO₂R¹R², NR¹SO₂R², C(O)R¹, C(O)OR¹, C(O)NR¹R². NR¹C(O)R², NR¹C(O)OR², CF₃, and OCF₃;
wherein Y is selected from the group consisting of O, S, NR¹ and CR¹R²;
wherein R¹ and R² are selected from the group consisting of hydrogen, nitro, hydroxyl, carboxy, amino, halogen, cyano and C₁-C₁₄ linear or branched alkyl groups, that are optionally substituted with 1-3 substituents selected from the group consisting of C₁-C₁₄ linear or branched alkyl, up to perhalo substituted C₁-C₁₄ linear or branched alkyl, C₁-C₁₄ alkoxy, hydrogen, nitro, hydroxyl, carboxy, amino, C₁-C₁₄ alkylamino, C₁-C₁₄ dialkylamino, halogen, and cyano; and
wherein Z is selected from the group consisting of hydrogen; C₁-C₁₄ linear, branched, or cyclic alkyls; phenyl; benzyl, 1-5 substituted benzyl. C₁ to C₃ alkyl-phenyl, wherein the alkyl moiety is optionally substituted with halogen up to perhalo; up to perhalo substituted C₁ to C₁₄ linear or branched alkyls; —(CH₂)q-K, where K is a 5 or 6 membered monocyclic heterocyclic ring, containing 1 to 4 atoms selected from oxygen, nitrogen and sulfur, which is saturated, partially saturated, or aromatic, or an 8 to 10 membered bicyclic heteroaryl having 1-4 heteroatoms selected from the group consisting of O, N, and S, wherein said alkyl moiety is optionally substituted with halogen up to perhalo, and wherein the variable q is an integer ranging from 0 to 4,
as described and defined in WO 2019/165229, the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a Ubr1 binding moiety as described in Shanmugasundaram, K. et al, J. Bio. Chem, 2019. doi: 10.1074/jbc.AC119.010790, the entirety of each of which is herein incorporated by reference, thereby forming a compound of formula I-sss-1 or I-sss-2:

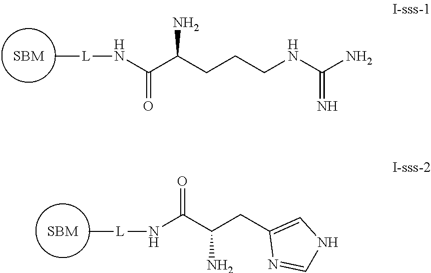

or a pharmaceutically acceptable salt thereof, wherein L and SBM are as defined above and described in embodiments herein.

In certain embodiments, the present invention provides methods of degrading STAT6, or a mutant thereof, or methods of treating a STAT6-mediated disease, disorder, or condition, comprising administering a compound of formula I, wherein LBM is a CRBN E3 ubiquitin ligase binding moiety thereby forming a compound of formula I-uuu-1, I-uuu-2, I-uuu-3 or I-uuu-4:

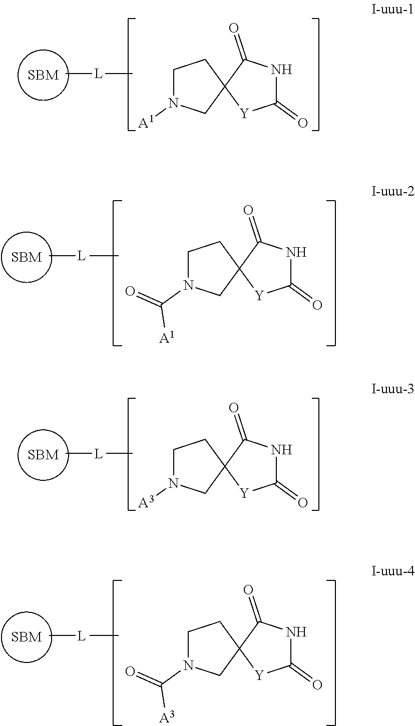

or a pharmaceutically acceptable salt thereof wherein L and SBM are as defined above and described in embodiments herein, and wherein:
Y is NH or CH₂:
A¹ is selected from the group consisting of aryl and aryl substituted with R¹;
A³ is selected from the group consisting of heteroaryl and heteroaryl substituted with R²;
R¹ is selected from the group consisting of; —C(═O)—O—C₁₋₆-alkyl, —COOH, —NH—(C═O)—C₁₋₆-alkyl, —NH₂, and —NO₂;
R² is selected from the group consisting of; —COOH, —C(═O)—O—C₁₋₆-alkyl, —NH₂, and —NO₂;
as described and defined in WO 2019/236483, the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is human kelch-like ECH-associated protein 1 (KEAP1) thereby forming a compound of formula I-vvv:

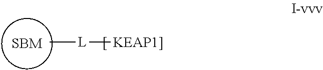

or a pharmaceutically acceptable salt thereof, wherein L and SBM are as defined above and described in embodiments herein, both singly and in combination.

In certain embodiments, the present invention provides methods of degrading STAT6, or a mutant thereof, or methods of treating a STAT6-mediated disease, disorder, or condition, comprising administering a compound of formula I, wherein LBM is KEAP1 binding moiety as recited in Lu et al., Euro. J. Med. Chem., 2018, 146:251-9, thereby forming a compound of formula I-www:

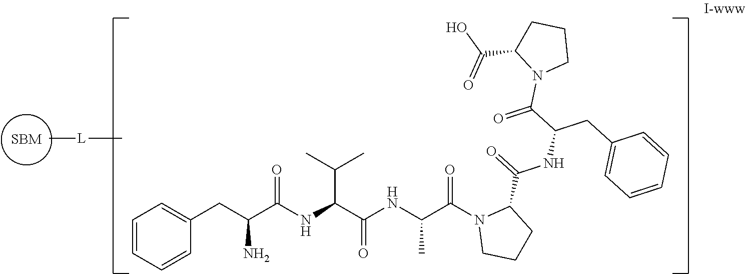

or a pharmaceutically acceptable salt thereof, wherein L and SBM are as defined above and described in embodiments herein, both singly and in combination.

In certain embodiments, the present invention provides methods of degrading STAT6, or a mutant thereof, or methods of treating a STAT6-mediated disease, disorder, or condition, comprising administering a compound of formula I, wherein LBM is KEAP1-NRF2 binding moiety thereby forming a compound of formula I-xxx-1 or I-xxx-2:

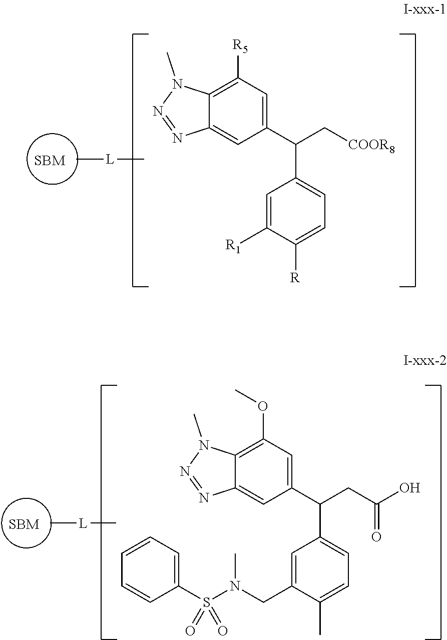

or a pharmaceutically acceptable salt thereof, wherein L and SBM are as defined above and described in embodiments herein, and wherein:
R is methyl or halo;
R¹ is

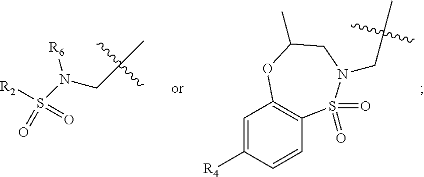

R² is methyl, or

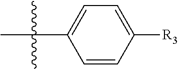

R³ is H;
R⁴ is H or halo;
R⁵ is methoxy or H:
R⁶ is H or methyl;
R⁸ is H, methyl or ethyl;
as described and defined in WO 2020/018788, the entirety of each of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-yyy-1 or I-yyy-2:

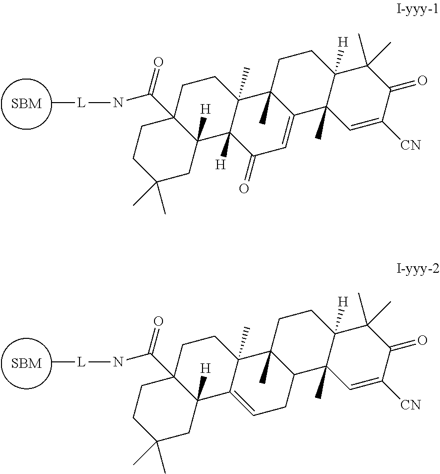

or a pharmaceutically acceptable salt thereof, wherein L and SBM are as defined above and described in embodiments herein, both singly and in combination.

In some embodiments, LBM is

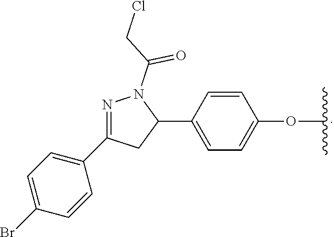 [SMILES: *Oc1ccc(C2CC(c3ccc(Br)cc3)=NN2C(=O)CCl)cc1]

In some embodiments, LBM is NH

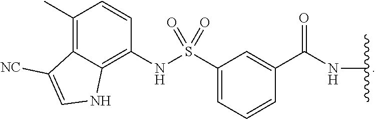 [SMILES: *NC(=O)c1cccc(S(=O)(=O)Nc2ccc(C)c3c(C#N)c[nH]c23)c1]

In some embodiments, the present disclosure provides methods of degrading STAT6, or a mutant thereof, or methods of treating a STAT6-mediated disease, disorder, or condition, comprising administering a compound of formula I-zzz:

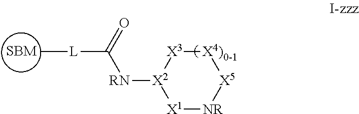

or a pharmaceutically acceptable salt thereof, wherein SBM, L, R, X¹, X², X³, X⁴, and X⁵ are as defined above and described herein.

In some embodiments, the present disclosure provides methods of degrading STAT6, or a mutant thereof, or methods of treating a STAT6-mediated disease, disorder, or condition, comprising administering a compound of formula I-aaaa:

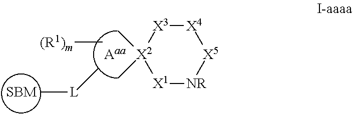

or a pharmaceutically acceptable salt thereof, wherein SBM, L, R, R¹, m, X¹, X², X³, X⁴, and X⁵ are as defined above and described herein; and: Ring A^{aa} is a 3- to 10-membered saturated or partially unsaturated carbocyclyl or heterocyclyl ring having 1-4 heteroatoms independently selected from nitrogen, oxygen, or sulfur.

As defined above and described herein, Ring A^{aa} is a 3- to 10-membered saturated or partially unsaturated carbocyclyl or heterocyclyl ring having 1-4 heteroatoms independently selected from nitrogen, oxygen, or sulfur. In some embodiments, Ring A^{aa} is a 3- to 10-membered saturated or partially unsaturated carbocyclyl. In some embodiments, Ring A^{aa} is a 3- to 10-membered saturated or partially unsaturated heterocyclyl ring having 1-4 heteroatoms independently selected from nitrogen, oxygen, or sulfur. In some embodiments, Ring A^{aa} is a 5- to 6-membered saturated or partially unsaturated carbocyclyl. In some embodiments, Ring A^{aa} is a 5- to 6-membered saturated or partially unsaturated heterocyclyl ring having 1-4 heteroatoms independently selected from nitrogen, oxygen, or sulfur.

In some embodiments, Ring A^{aa} is a 5-membered saturated or partially unsaturated heterocyclyl ring having 1-4 heteroatoms independently selected from nitrogen, oxygen, or sulfur. In some embodiments, Ring A^{aa} is pyrrolidinyl. In some embodiments, Ring A^{aa} is 4,5-dihydrothiazolyl. In some embodiments, Ring A^{aa} is 4,5-dihydroisoxazolyl.

In some embodiments, a compound of formulae I-aaaa is of formula I-aaaa-1, I-aaaa-2, or I-aaaa-3:

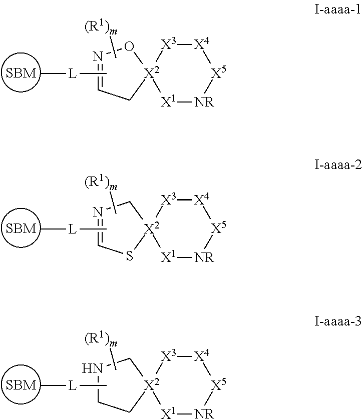

or a pharmaceutically acceptable salt thereof, wherein SBM, L, R, R¹, m, X¹, X², X³, X⁴, and X⁵ are as defined above and described herein.

In certain embodiments, the present invention provides methods of degrading STAT6, or a mutant thereof, or methods of treating a STAT6-mediated disease, disorder, or condition, comprising administering a compound of formula I-b:

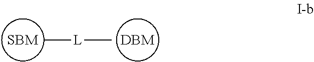

or a pharmaceutically acceptable salt thereof, wherein, SBM and L are as defined above and described herein; and
DBM is DCAF E3 ubiquitin ligase binding moiety capable of binding to DCAF1 protein, as described below and herein.

As described above, in certain embodiments, the present invention provides methods of degrading STAT6, or a mutant thereof, or methods of treating a STAT6-mediated disease, disorder, or condition, comprising administering a compound of formula I-b, wherein DBM is a compound of formula I-b-a or I-b-b:

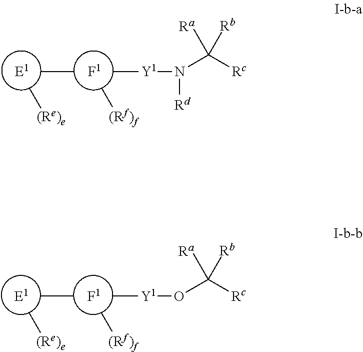

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.
Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula I-b-a where R^{d} is absent;
R^{a} is hydrogen, an optionally substituted C₁₋₆ aliphatic, or

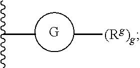

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CR₂S(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur; a 5-9 membered monocyclic or bicyclic heteroaryl with 1-4 heteroatoms independently selected from nitrogen, oxygen and sulfur; or:
   (CR₂)₁₋₂—X^{a}, wherein X^{a} is halogen or an optionally substituted ring selected from phenyl; a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or:
   R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R^{b} and R^{c} are taken together with their intervening atoms to form an optionally substituted phenyl; or:
   when Y¹ is —C(OR)═, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R^{c} is —CR₂CONR₂, Rd is taken together with a single R of —CR₂CONR₂ with their intervening atoms to form a 5-7 membered saturated or partially unsaturated heterocyclyl with 0-3 heteroatoms, in addition to the nitrogen atom to which Rd is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R², —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂:
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

As described above, in certain embodiments, the present invention provides methods of degrading STAT6, or a mutant thereof, or methods of treating a STAT6-mediated disease, disorder, or condition, comprising administering a compound of formula I-b, wherein DBM is a compound of formula I-b-c:

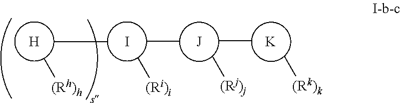

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl or a 5-10 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R—OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an R¹ group on Ring I and an R^{J} group or Ring J are optionally taken together with their intervening atoms to form a 5-8 membered saturated, partially unsaturated, or aromatic ring having 0-3 heteroatoms independently selected from nitrogen, oxygen, and sulfur, or:
each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

In certain embodiments, the present invention provides methods of degrading STAT6, or a mutant thereof, or methods of treating a STAT6-mediated disease, disorder, or condition, comprising administering a compound of formula I-b-c as any one of the following formula:

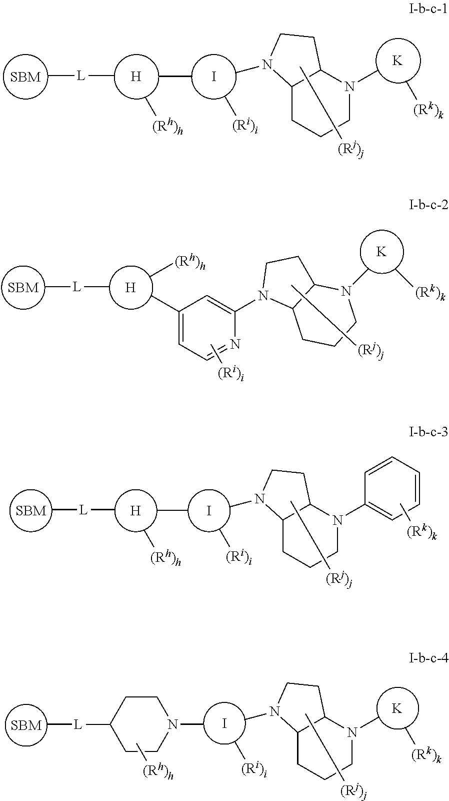

or a pharmaceutically acceptable salt thereof, wherein each of the variables is as defined above in formula I-b-c and described in embodiments herein, both singly and in combination.

In certain embodiments, the present invention provides methods of degrading STAT6, or a mutant thereof, or methods of treating a STAT6-mediated disease, disorder, or condition, comprising administering a compound of formula I-c-a-1 or I-c-a-2 as any one of the following formula:

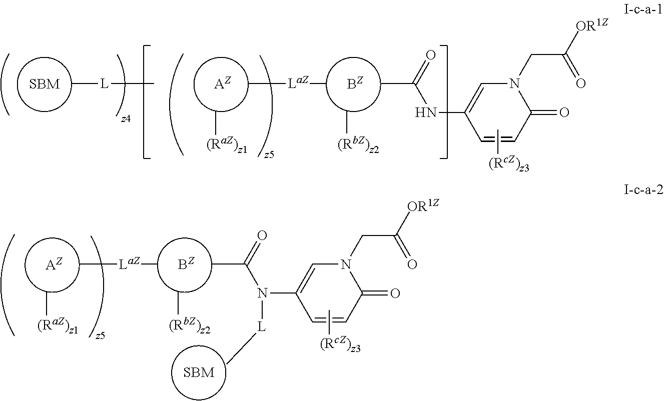

or a pharmaceutically acceptable salt thereof, wherein:
SBM and L are as defined and described above and herein;
R^{1Z} is hydrogen or optionally substituted C₁₋₆ aliphatic;
each R^{aZ}, R^{bZ}, and R^{cZ} are independently hydrogen, R^{Z}, halogen, —CN, —NO₂, —OR^{Z}—SR^{Z}—NR^{Z}₂, —S(O)₂R^{Z}, —S(O)₂NR^{Z}₂, —S(O)R^{Z}—S(O)(NR^{Z})R^{Z}, —P(O)(OR^{Z})₂, —P(O)(NR^{Z}₂)₂, —CFR^{Z}₂, —CR^{Z}F₂, —CF₃, —CR^{Z}₂(OR^{Z}), —CR₂(NR₂), —C(O)R^{Z}, —C(O)OR^{Z}, or —C(O)NR^{Z}₂;
each R^{AZ} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{Z} is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{Z} groups on the same atom are optionally taken together with their intervening atom to form an optionally substituted 4-11 membered saturated or partially unsaturated carbocyclic or
heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
each Ring A^{Z} is independently a bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each Ring B^{Z} is independently a bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
L^{aZ} is absent, a covalent bond, or a C₁₋₃ bivalent straight or branched saturated or unsaturated hydrocarbon chain, wherein 1-3 methylene units of the chain are independently and optionally replaced with —O—, —C(O)—, —C(S)—, —C(R^{Z})₂—, —CH(R^{Z})—, —CF(R^{Z})—, —C(F)₂, —N(R^{Z})—, —S—, —S(O)₂— or —CR^{Z}═CR^{Z}—:
z1, z2, and z3 are each independently 0, 1, 2, 3 or 4;
each of z4 and z5 is independently 0 or 1.

In some embodiments, methods of degrading STAT6, or a mutant thereof, or methods of treating a STAT6-mediated disease, disorder, or condition, comprising administering a compound of formula I-c-a-1, or a pharmaceutically acceptable salt thereof. In some embodiments, methods of degrading STAT6, or a mutant thereof, or methods of treating a STAT6-mediated disease, disorder, or condition, comprising administering a compound is of formula I-c-a-2, or a pharmaceutically acceptable salt thereof.

In some embodiments, R^{1Z} is hydrogen, methyl, or ethyl. In some embodiments, R^{1Z} is hydrogen.

In some embodiments, each Ring Az is independently a bivalent ring selected from phenylenyl, naphthalenyl, or a 5-10 membered monocyclic or bicyclic heteroarylenyl having 1-4 heteroatoms independently selected from nitrogen, oxygen, and sulfur. In some embodiments, each Ring B is independently a bivalent ring selected from phenylenyl, a 5-6 membered saturated or partially unsaturated monocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In certain embodiments, the present invention provides methods of degrading STAT6, or a mutant thereof, or methods of treating a STAT6-mediated disease, disorder, or condition, comprising administering a compound of formula I, wherein LBM is a CRBN E3 ubiquitin ligase binding moiety thereby forming a compound of formula I-bbbb-1:

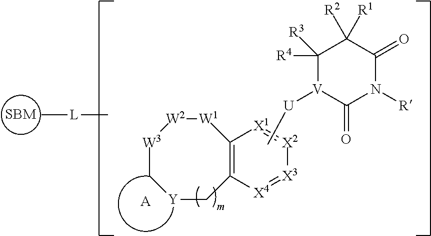

or a pharmaceutically acceptable salt thereof, wherein L and SBM are as defined above and described in embodiments herein, and
each additional variable as described and defined in CN 118005655 A, the entirety of which is herein incorporated by reference.

In certain embodiments, the present invention provides methods of degrading STAT6, or a mutant thereof, or methods of treating a STAT6-mediated disease, disorder, or condition, comprising administering a compound of formula I, wherein LBM is a CRBN E3 ubiquitin ligase binding moiety thereby forming a compound of formula I-cccc-1:

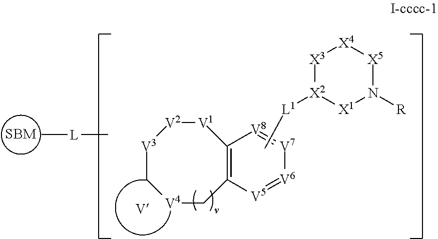

or a pharmaceutically acceptable salt thereof, wherein L, SBM, X¹, X², X³, X⁴, X⁵, R, and L¹ are as defined above and described in embodiments herein, wherein:
One of V⁵, V⁶, V⁷, and V⁸ is a carbon atom which is attached to L¹, and the others are independently selected from N or CR^{V};
each R^{V} is independently selected from hydrogen, halogen, —OR, —NR₂, —CN, or an optionally substituted group selected from C₁₋₆ aliphatic, a 3-8 membered saturated or partially unsaturated carbocyclyl or heterocyclyl having 1-3 heteroatom independently selected from nitrogen, oxygen, or sulfur, phenyl, or a 5-6 membered heteroaryl having 1-3 heteroatom independently selected from nitrogen, oxygen, or sulfur;
each of V¹, V², and V³ is independently selected form a bond, —S—, —S(O)—, —S(O)₂—, —C(O)—, —C(R^{V}₂)—, —(C(R^{V}₂))₂—, or —N(R^{V}),
V⁴ is selected from N, C, or CR^{V}; and
Ring V′ is an optionally substituted fused saturated or partially unsaturated carbocyclyl or heterocyclyl having 1-3 heteroatom independently selected from nitrogen, oxygen, or sulfur.

### Degradation Inducing Moiety (DIM)

In certain embodiments, the present invention provides methods of degrading STAT6, or a mutant thereof, or methods of treating a STAT6-mediated disease, disorder, or condition, comprising administering a compound of formula I:

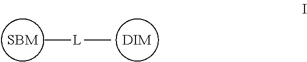

or a pharmaceutically acceptable salt thereof, wherein L and SBM are as described above and herein within this section (3a), and DIM is a degradation inducing moiety selected from LBM, a lysine mimetic, or a hydrogen atom.

In some embodiments, DIM is LBM as described above and herein. In some embodiments, DIM is a lysine mimetic. In some embodiments, the covalent attachment of ubiquitin to STAT6 protein is achieved through the action of a lysine mimetic. In some embodiments, upon the binding of a compound of formula I to STAT6 protein, the moiety that mimics a lysine undergoes ubiquitination thereby marking STAT6 protein for degradation via the Ubiquitin-Proteasome Pathway (UPP).

In some embodiments, DIM is

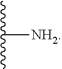 [SMILES: *N]

In some embodiments, DIM is

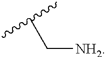 [SMILES: *CN]

In some embodiments, DIM is

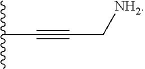 [SMILES: *C#CCN]

In some embodiments, DIM is selected from those depicted in Table 2, below.

In some embodiments, the present invention provides methods of degrading STAT6, or a mutant thereof, or methods of treating a STAT6-mediated disease, disorder, or condition, comprising administering a compound of formula I as a compound of formula I-aaaa:

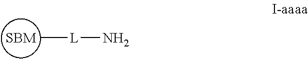

or a pharmaceutically acceptable salt thereof, wherein each of SBM and L is as defined above and described in embodiments herein, both singly and in combination.

In some embodiments, the present invention provides methods of degrading STAT6, or a mutant thereof, or methods of treating a STAT6-mediated disease, disorder, or condition, comprising administering a compound of formula I as a compound of formula I-aaaa-1:

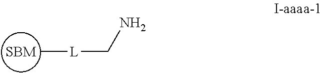

or a pharmaceutically acceptable salt thereof, wherein each of SBM and L is as defined above and described in embodiments herein, both singly and in combination.

In some embodiments, the present invention provides methods of degrading STAT6, or a mutant thereof, or methods of treating a STAT6-mediated disease, disorder, or condition, comprising administering a compound of formula I as a compound of formula I-aaaa-2:

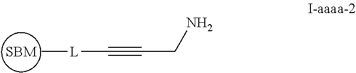

or a pharmaceutically acceptable salt thereof, wherein each of SBM and L is as defined above and described in embodiments herein, both singly and in combination.

In certain embodiments, the present invention provides methods of degrading STAT6, or a mutant thereof, or methods of treating a STAT6-mediated disease, disorder, or condition, comprising administering a compound of Formula I, wherein DIM is a lysine mimetic

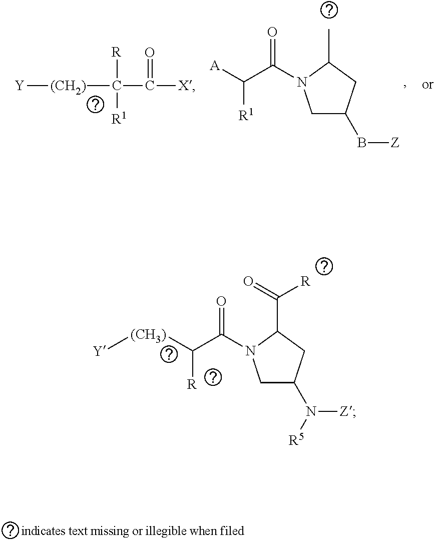

thereby forming a compound of Formulae I-bbbb-1, I-bbbb-2, or I-bbbb-3, respectively:

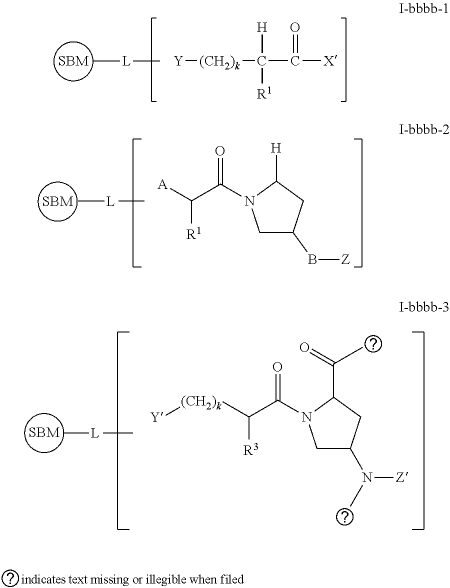

or a pharmaceutically acceptable salt thereof, wherein L and SBM are as defined above and described in embodiments herein, and wherein:
A is (CH₂)ₖ—Y;
k is 0, 1, or 2;
Y′ is OR² or NR²R³;
R¹ is selected from H, an optionally substituted C₁₋₁₀alkyl, an optionally substituted C₆₋₂₀aryl, an optionally substituted C₇₋₂₀aralkyl, and an amino acid side chain;
alternatively, A and R¹ together with the carbon atom to which they are bound form a 5-20 membered heteroaryl containing 1-4 ring heteroatoms independently selected from N, O, and S and optionally substituted with 1-5 Q groups:
B is selected from NR⁵, NR(CH₂)ₙC(O), NR⁵(CH₂)ₙ, S(O)₂, and an amide bioisostere;
n is 0, 1, or 2;
Z is selected from H, (CH₂)ₘ—C₆₋₂₀ aryl optionally substituted with 1-5 Q groups, and (CH₂)ₘ-5-20 membered heteroaryl optionally substituted with 1-5 Q groups;
Z′ is selected from H, (CH₂)ₘ—C₆₋₂₀ aryl, (CH₂)ₘ-5-20 membered heteroaryl, C(O)(CH₂)ₘ—C₆₋₂₀ aryl, C(O)(CH₂)ₘ-5-20 membered heteroaryl, (CH₂)ₘC(O)—C₆₋₂₀ aryl, (CH₂)ₘC(O)-5-20 membered heteroaryl, S(O)₂(CH₂)ₘ—C₆₋₂₀ aryl, and S(O)₂(CH₂)ₘ-5-20 membered heteroaryl, wherein each of the C₆₋₂₀ aryl and 5-20 membered heteroaryl is optionally substituted with 1-5 Q groups;
m is 0, 1, or 2;
E is selected from C(O)OR⁶, C(O)NR⁶R⁷, a carboxylic acid bioisostere and an amide bioisostere;
Q, at each occurrence, independently is selected from an optionally substituted C₁₋₁₀ alkyl, an optionally substituted C₂₋₁₀ alkenyl, an optionally substituted C₆₋₁₀ alkynyl, an optionally substituted C3-20 cycloalkyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, F, Cl, Br, I, CN, CF₃, OCF₃, NO₂, OR⁸, SR, S⁺R⁸₂, S(O)R⁸, S(O)₂R⁸, S(O)₂OH, S(O)₂NR⁸R⁹, NR^{B}S(O)₂R⁹, C(O)R, C(O)OR⁸, C(O)NR⁸R⁹, OC(O)R, NR⁸R⁹, NRC(O)R⁹, NR⁸C(O)OR₉, NR⁸C(O)NR⁸R⁹, and N⁺R⁸₃;
R² and R³ each independently is selected from H, an optionally substituted C₁₋₁₀ alkyl, an optionally substituted C₃₋₂₀ cycloalkyl, an optionally substituted C₇₋₂₀ aralkyl, an optionally substituted C₆₋₂₀ aryl, an optionally substituted 3-20 membered cycloheteroalkyl, an optionally substituted 5-20 membered heteroaryl, C(O)R⁶, C(O)OR⁶, C(O)NR⁶R⁷, S(O)₂R⁶, and S(O)₂NR⁶R⁷;
alternatively, R² and R³ together with the nitrogen atom to which they are bound form a 3-20 membered heterocycle optionally containing 1-4 ring heteroatoms independently selected from O, N and S atoms and optionally substituted with 1-5 Q groups:
R⁵ is H or an optionally substituted C₁₋₁₀ alkyl:
R⁶ and R⁷ each independently is selected from H, an optionally substituted C₁₋₁₀ alkyl, an optionally substituted C₃₋₂₀ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, C(O)R⁸. C(O)OR⁸, and C(O)NR⁸R⁹:
alternatively, R⁶ and R₇ together with the nitrogen atom to which they are bound form a 3-20 membered heterocycle optionally containing 1-4 ring heteroatoms independently selected from O, N and S and optionally substituted with 1-5 Q groups; and
R⁸ and R⁹ each independently is selected from H, an optionally substituted C, alkyl, an optionally substituted C³⁻²⁰ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, and an optionally substituted 5-20 membered heteroaryl, provided that the compound is not 1-(2-aminopropanoyl)-4-benzamidopyrrolidine-2-carboxylic acid.
as defined and described in U.S. Pat. No. 7,622,496, the entirety of each of which is herein incorporated by reference.

### Linker (L)

As defined above and described herein, L is a bivalent moiety that connects SBM to LBM or SBM to DIM.

In some embodiments, L is a bivalent moiety that connects SBM to LBM. In some embodiments, L is a bivalent moiety that connects SBM to DIM. In some embodiments, L is a bivalent moiety that connects SBM to a lysine mimetic.

In some embodiments, L is a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

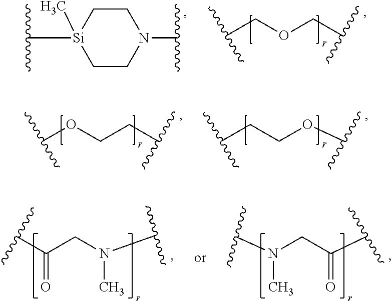

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

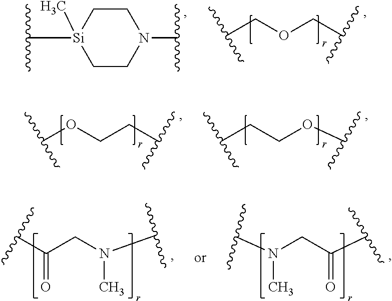

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or a bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —C(O)NR—, —OC(O)NR—, —NRC(O)O—,

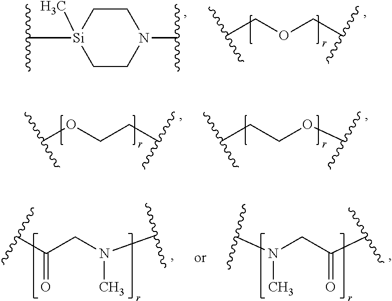

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋₂₀ hydrocarbon chain, wherein 0-4 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

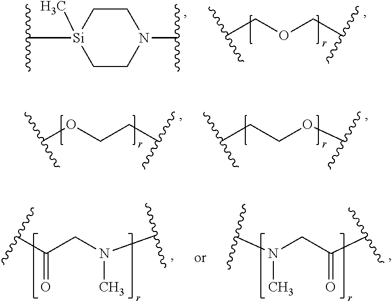

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond. In some embodiments, L is a bivalent, saturated or partially unsaturated, straight or branched C₁₋₅₀, C₁₋₄₀, C₁₋₃₀, C₁₋₂₀, or C₁₋₁₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

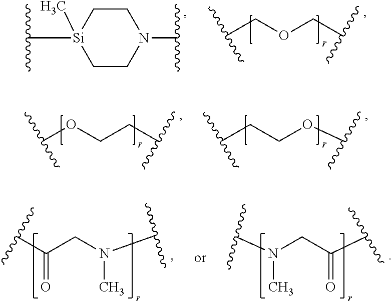

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, —NRC(O)O—,

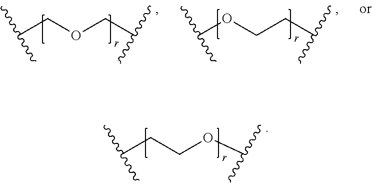

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

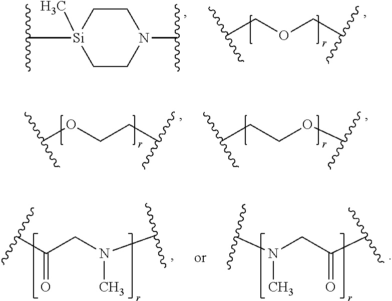

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially saturated, straight or branched C₁₋₁₀ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

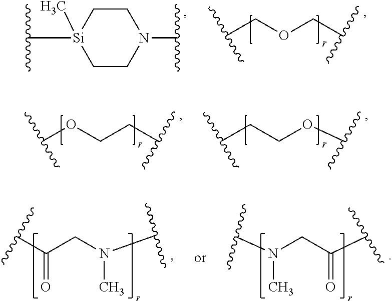

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

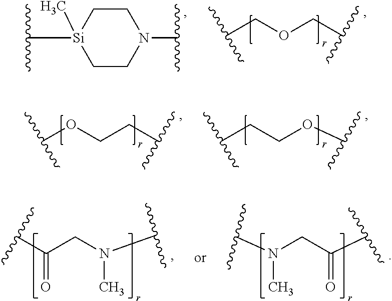

In some embodiments, L is an optionally substituted bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

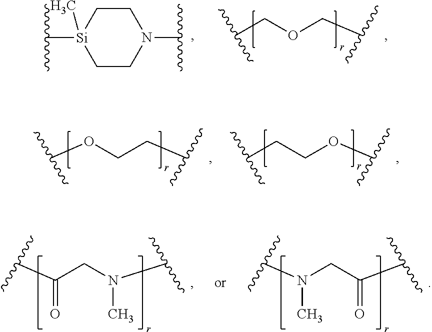

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₂ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, or NRC(O)O—, provided that L does not comprise —NR—C(O)—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —S—, —C(O)O—, —C(O)—, —S(O)—, —S(O)₂—, —NRS(O)₂—, —NRC(O)—, or NRC(O)O—.

In some embodiments, L is —NR—(C₁₋₁₀aliphatic)-. In some embodiments, L is —(C₁₋₁₀aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C-oo aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is —(C₁₋₁₀ aliphatic)-. In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is -Cy-(optionally substituted C₁₋₁₀ aliphatic)-. In some embodiments, L is -Cy-(optionally substituted C₁₋₁₀ aliphatic)-Cy-. In some embodiments, L is -Cy-Cy-(optionally substituted C₁₋₁₀ aliphatic)-. In some embodiments, L is -Cy-Cy-Cy-(optionally substituted C₁₋₁₀ aliphatic)-. In some embodiments, L is -Cy-Cy-(optionally substituted C₁₋₁₀ aliphatic)-Cy-. In some embodiments, L is -Cy-(C₁₋₁₀ aliphatic)-. In some embodiments, L is -Cy-Cy-(C₁₋₁₀ aliphatic)-. In some embodiments, L is -Cy-Cy-Cy-(C₁₋₁₀ aliphatic)-. In some embodiments, L is -Cy-Cy-(C₁₋₁₀ aliphatic)-Cy-.

In some embodiments, L is -Cy-(optionally substituted C₁₋₆ aliphatic)-. In some embodiments, L is -Cy-(optionally substituted C₁₋₆ aliphatic)-Cy-. In some embodiments, L is -Cy-Cy-(optionally substituted C₁₋₆ aliphatic)-. In some embodiments, L is -Cy-Cy-Cy-(optionally substituted C₁₋₆ aliphatic)-. In some embodiments, L is -Cy-Cy-(optionally substituted C₁₋₆ aliphatic)-Cy-. In some embodiments, L is -Cy-(C₁₋₆ aliphatic)-. In some embodiments, L is -Cy-(C₁₋₆ aliphatic)-Cy-. In some embodiments, L is -Cy-Cy-(C₁₋₆ aliphatic)-. In some embodiments, L is -Cy-Cy-Cy-(C₁₋₆ aliphatic)-. In some embodiments, L is -Cy-Cy-(C₁₋₆ aliphatic)-Cy-.

In some embodiments, L is -Cy-(optionally substituted C₁₋₃ aliphatic)-. In some embodiments, L is -Cy-(optionally substituted C₁₋₃ aliphatic)-Cy-. In some embodiments, L is -Cy-Cy-(optionally substituted C₁₋₃ aliphatic)-. In some embodiments, L is -Cy-Cy-Cy-(optionally substituted C₁₋₃ aliphatic)-. In some embodiments, L is -Cy-Cy-(optionally substituted C₁₋₃ aliphatic)-Cy-. In some embodiments, L is -Cy-(C₁₋₃ aliphatic)-. In some embodiments, L is -Cy-(C₁₋₃ aliphatic)-Cy-. In some embodiments, L is -Cy-Cy-(C₁₋₃ aliphatic)-. In some embodiments, L is -Cy-Cy-Cy-(C₁₋₃ aliphatic)-. In some embodiments, L is -Cy-Cy-(C₁₋₃ aliphatic)-Cy-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀. In some embodiments, L is —(CH₂)₁₋₁₀-Cy-CONR—(CH₂)₁₋₁₀. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀CONR—(CH₂)₁₋₁₀. In some embodiments, L is -Cy-(CH₂)₁₋₁₀-Cy-CONR—. In some embodiments, L is -Cy-(CH₂)₁₋₁₀CONR-Cy-. In some embodiments, L is -Cy-(CH₂)₁₋₁₀-Cy-CONR—(CH₂)₁₋₁₀-. In some embodiments, L is -Cy-(CH₂)l-O—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₀—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)l-o-O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)-o-O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—. In some embodiments, L is -Cy-Cy-. In some embodiments, L is -Cy-Cy-(CH₂)-o-. In some embodiments, L is -Cy-(CH₂)₁₋₁₀-Cy-(CH₂)₁₋₁₀—. In some embodiments, L is -Cy-Cy-Cy-. In some embodiments, L is -Cy-Cy-(CH₂)₁₋₁₀-Cy-. In some embodiments, L is -Cy-Cy-(CH₂)₁₋₁₀-Cy-(CH₂)₁₋₁₀—.

In some embodiments, L is optionally substituted -Cy-(CH₂)₁₋₁₀—. In some embodiments, L is optionally substituted —(CH₂)₁₋₁₀-Cy-(CH₂)₁₋₁₀—. In some embodiments, L is optionally substituted —(CH₂)₁₋₁₀-Cy-(CH₂CH₂O)₁₋₁₀CH₂CH₂—. In some embodiments, L is optionally substituted -Cy-(CH₂)₁₋₁₀-Cy-. In some embodiments, L is optionally substituted -Cy-(CH₂)₁₋₁₀-Cy-(CH₂)₁₋₁₀—. In some embodiments, L is optionally substituted -Cy-(CH₂)₁₋₁₀-Cy-(CH₂)₁₋₁₀-Cy-. In some embodiments, L is optionally substituted —(CH₂)₁₋₁₀-Cy-(CH₂)₁₋₁₀-Cy-(CH₂)₁₋₁₀—. In some embodiments, L is optionally substituted -Cy-Cy-. In some embodiments, L is optionally substituted -Cy-Cy-(CH₂)₁₋₁₀—. In some embodiments, L is optionally substituted -Cy-(CH₂)₁₋₁₀-Cy-(CH₂)₁₋₁₀—. In some embodiments, L is optionally substituted -Cy-Cy-Cy-. In some embodiments, L is optionally substituted -Cy-Cy-(CH₂)-w-Cy-. In some embodiments, L is optionally substituted -Cy-Cy-(CH₂)₁₋₁₀-Cy-(CH₂)₁₋₁₀—.

In some embodiments, L comprises one -Cy- group. In some embodiments, L comprises two -Cy- groups. In some embodiments, L comprises three -Cy- groups. In some embodiments, L does not comprise a -Cy- group.

In some embodiments, L comprises —C(O)—.

In some embodiments, L comprises one -Cy- group and one methylene group. In some embodiments, L comprises two -Cy- groups and one methylene group. In some embodiments, L comprises one -Cy- group and two methylene groups.

In some embodiments, L is an optionally substituted bivalent, saturated or partially unsaturated, straight or branched C₁₋₁₀ hydrocarbon chain, wherein 1-4 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by -Cy-, and 1-2 additional methylene units of L are optionally and independently replaced with —CHF—, —CF₂—, O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 2 methylene units of L are independently replaced by -Cy-, and 1 additional methylene unit of L is optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 3 methylene units of L are independently replaced by -Cy-. In some embodiments, L is an optionally substituted bivalent, saturated or partially unsaturated, straight or branched C₁₋₁₀ hydrocarbon chain, wherein 1 methylene unit of L is replaced by —C(O)—, and 1-2 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is:

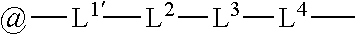

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-, Cy^{L2}-, -Cy^{L3}-, and -Cy^{L4}- is independently -Cy-, wherein -Cy- is as defined above and described herein.

In some embodiments, L is -@-L¹-L²-L³-. In some embodiments, L is @-L¹-L²-. In some embodiments, L is @-L¹-.

In some embodiments, L¹ is a covalent bond or an optionally substituted bivalent, saturated or partially unsaturated, straight or branched C₁₋₆ hydrocarbon chain, wherein 1 methylene unit of L¹ is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L¹ is a covalent bond. In some embodiments, L¹ is an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L¹ is -Cy^{L1}-. In some embodiments, L¹ is an optionally substituted bivalent, saturated or partially unsaturated, straight or branched C₁₋₆ hydrocarbon chain. In some embodiments, L¹ is an optionally substituted bivalent, saturated or partially unsaturated, straight or branched C₁₋₃ hydrocarbon chain. In some embodiments, L^{w} is an optionally substituted bivalent, saturated or partially unsaturated, straight or branched C₁₋₆ hydrocarbon chain, wherein 1 methylene unit of L¹ is optionally and independently replaced by —O— or —NR—. In some embodiments, L¹ is —O—. In some embodiments, L¹ is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L¹ is —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—.

In some embodiments, L² is a covalent bond or an optionally substituted bivalent, saturated or partially unsaturated, straight or branched C₁₋₆ hydrocarbon chain, wherein 1 methylene unit of L¹ is optionally replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L² is a covalent bond. In some embodiments, L² is an optionally substituted bivalent, saturated or partially unsaturated, straight or branched C₁₋₆ hydrocarbon chain, wherein 1-2 methylene units of L² are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L² is -Cy^{L2}-. In some embodiments, L² is an optionally substituted bivalent, saturated or partially unsaturated, straight or branched C₁₋₆ hydrocarbon chain. In some embodiments, L² is an optionally substituted bivalent, saturated or partially unsaturated, straight or branched C₁₋₃ hydrocarbon chain. In some embodiments, L² is —O—. In some embodiments, L² is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L² is —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—.

In some embodiments, L³ is a covalent bond or an optionally substituted bivalent, saturated or partially unsaturated, straight or branched C₁₋₆ hydrocarbon chain, wherein 1 methylene unit of L¹ is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L³ is a covalent bond. In some embodiments, L³ is an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L³ is -Cy^{L3}-. In some embodiments, L³ is an optionally substituted bivalent, saturated or partially unsaturated, straight or branched C₁₋₆ hydrocarbon chain. In some embodiments, L³ is an optionally substituted bivalent, saturated or partially unsaturated, straight or branched C₁₋₃ hydrocarbon chain. In some embodiments, L³ is —O—. In some embodiments, L³ is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L³ is —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—.

In some embodiments, L⁴ is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L⁴ is a covalent bond. In some embodiments, L⁴ is an optionally substituted bivalent, saturated or partially unsaturated, straight or branched C₁₋₆ hydrocarbon chain, wherein 1-2 methylene units of L⁴ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L⁴ is -Cy^{L1}-. In some embodiments, L⁴ is an optionally substituted bivalent, saturated or partially unsaturated, straight or branched C₁₋₆ hydrocarbon chain. In some embodiments, L⁴ is an optionally substituted bivalent, saturated or partially unsaturated, straight or branched C₁₋₃ hydrocarbon chain. In some embodiments, L⁴ is —O—. In some embodiments, L⁴ is optionally substituted —CH₂—, —CH₂CH₂—, —CH═CH—, —C═C—, —CH₂CH₂CH₂—, or —CH═CHCH₂—, —CH₂CH═CH—, —C—CCH₂—, or —CH₂C═C—. In some embodiments, L⁴ is —CH₂—, —CH₂CH₂—, —CH═CH—, —CC—, —CH₂CH₂CH₂—, or —CH═CHCH₂—, —CH₂CH═CH—, —C═CCH₂—, or —CH₂C═C—.

As defined above and described herein, each of -Cy^{L2}-. Cy^{L2}, -Cy^{L3}-, and -Cy^{L1}- is independently -Cy-, wherein -Cy- is as defined above and described herein.

In some embodiments, -Cy^{L1}- is an optionally substituted 8-10 membered bicyclic arylenyl. In some embodiments, -Cy^{L1}- is an optionally substituted 3-7 membered saturated or partially unsaturated carbocyclylenyl. In some embodiments, -Cy^{L1}- is an optionally substituted 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L1}- is an optionally substituted a 4-7 membered saturated or partially unsaturated heterocyclylenyl having 1-2 heteroatoms independently selected from nitrogen, oxygen, and sulfur. In some embodiments, -Cy^{L1}- is an optionally substituted 6-11 membered saturated or partially unsaturated spiro heterocyclylenyl having 1-3 heteroatoms independently selected from nitrogen, oxygen, and sulfur. In some embodiments, -Cy^{L1}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L1}- is an optionally substituted 5-6 membered heteroarylenyl having 1-4 heteroatoms independently selected from nitrogen, oxygen. In some embodiments, -Cy^{L1}- is an optionally substituted 8-10 membered bicyclic heteroarylenyl having 1-5 heteroatoms independently selected from nitrogen, oxygen, and sulfur.

In some embodiments, each -Cy^{L1}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an 8-10 membered bicyclic arylenyl. In some embodiments, -Cy^{L1}- is a 3-7 membered saturated or partially unsaturated carbocyclylenyl. In some embodiments, -Cy^{L2}- is a 6-11 membered saturated or partially unsaturated spiro carbocyclylenyl. In some embodiments, -Cy^{L1}- is an 8-10 membered bicyclic saturated or partially unsaturated carbocyclylenyl. In some embodiments, -Cy^{L1}- is a 4-7 membered saturated or partially unsaturated heterocyclylenyl having 1-2 heteroatoms independently selected from nitrogen, oxygen, and sulfur. In some embodiments, -Cy^{L1}- is a 6-11 membered saturated or partially unsaturated spiro heterocyclylenyl having 1-3 heteroatoms independently selected from nitrogen, oxygen, and sulfur. In some embodiments, -Cy^{L1}- is an 8-10 membered bicyclic saturated or partially unsaturated heterocyclylenyl having 1-2 heteroatoms independently selected from nitrogen, oxygen, and sulfur. In some embodiments, -Cy^{L1}- is a 5-6 membered heteroarylenyl having 1-4 heteroatoms independently selected from nitrogen, oxygen. In some embodiments, -Cy- is an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L1}- is an optionally substituted phenylenyl. In some embodiments, -Cy^{L1}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L1}- is an optionally substituted 5-6 membered saturated or partially unsaturated heterocyclylenyl having 1-2 heteroatoms independently selected from nitrogen, oxygen, and sulfur. In some embodiments, -Cy- is an optionally substituted 5 membered saturated or partially unsaturated heterocyclylenyl having 1-2 heteroatoms independently selected from nitrogen, oxygen, and sulfur. In some embodiments, -Cy- is an optionally substituted pyrrolidinylenyl. In some embodiments, -Cy^{L1}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrrolidinylenyl, optionally substituted with halogen (e.g., fluoro or chloro) or C₁₋₆ aliphatic. In some embodiments, -Cy^{L1}- is an optionally substituted 6 membered saturated or partially unsaturated heterocyclylenyl having 1-2 heteroatoms independently selected from nitrogen, oxygen, and sulfur. In some embodiments, -Cy^{L2}- is an optionally substituted piperadinylenyl. In some embodiments, -Cy^{L1}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is piperadinylenyl, optionally substituted with halogen (e.g., fluoro or chloro) or C₁₋₆ aliphatic. In some embodiments, -Cy^{L1}- is an optionally substituted piperazinylenyl. In some embodiments, -Cy^{L1}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is piperazinylenyl, optionally substituted with halogen (e.g., fluoro or chloro) or C₁e aliphatic.

In some embodiments, -Cy^{L1}- is an optionally substituted 5 membered heteroarylenyl having 1-4 heteroatoms independently selected from nitrogen, oxygen. In some embodiments, -Cy^{L2}- is an optionally substituted pyrrolylenyl, pyrazolylenyl, imidazolylenyl, or triazolylenyl. In some embodiments, -Cy^{L1}- is pyrrolylenyl, pyrazolylenyl, imidazolylenyl, or triazolylenyl, optionally substituted with halogen (e.g., fluoro or chloro), —CN, C₁e aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L1}- is an optionally substituted pyrazolylenyl. In some embodiments, -Cy^{L2}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrazolylenyl, optionally substituted with halogen (e.g., fluoro or chloro) or C₁₋₆ aliphatic. In some embodiments, -Cy^{L1}- is an optionally substituted imidazolylenyl. In some embodiments, -Cy^{L1}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L2}- is imidazolylenyl, optionally substituted with halogen (e.g., fluoro or chloro) or C₁₋₆ aliphatic.

In some embodiments, -Cy^{L1}- is an optionally substituted 6 membered heteroarylenyl having 1-4 heteroatoms independently selected from nitrogen, oxygen. In some embodiments, -Cy^{L1}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L1}- is an optionally substituted pyridinylenyl. In some embodiments, -Cy^{L1}- is pyridinylenyl, optionally substituted with halogen (e.g., fluoro or chloro), —CN, C₁₋₆ aliphatic optionally further substituted with halogen (e.g., fluoro or chloro), —S(O)₂R^{o} or —OR^{o}, wherein R^{o} is hydrogen or C₁₋₆ aliphatic. In some embodiments, -C^{L1}- is pyridinylenyl, optionally substituted with halogen (e.g., fluoro or chloro) or C₁₋₆ aliphatic. In some embodiments, -Cy^{L1}- is an optionally substituted pyridazinylenyl. In some embodiments, -Cy^{L1}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L2}- is an optionally substituted pyridinonylenyl. In some embodiments, -Cy^{L1}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, optionally substituted with halogen (e.g., fluoro or chloro) or C₁₋₆ aliphatic.

In some embodiments, -Cy^{L1}- is an optionally substituted cyclopropyl. In some embodiments, -Cy^{L1}- is an optionally substituted 5 membered saturated or partially unsaturated carbocyclylenyl. In some embodiments, -Cy^{L1}- is an optionally substituted cyclopentanylenyl or cyclopentenylenyl. In some embodiments, -Cy^{L1}- is an optionally substituted 6 membered saturated or partially unsaturated carbocyclylenyl. In some embodiments, -Cy^{L1}- is an optionally substituted cyclohexanylenyl or cyclohexenylenyl. In some embodiments, -Cy^{L1}- is an optionally substituted naphthalenylenyl.

In some embodiments, -Cy^{L1}- is an optionally substituted 9 membered bicyclic heteroarylenyl having 1-5 heteroatoms independently selected from nitrogen, oxygen, and sulfur. In some embodiments, -Cy^{L2}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L1}- is an optionally substituted indolylenyl. In some embodiments, -Cy^{L1}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L1}- is an optionally substituted 8-11 membered saturated or partially unsaturated spiro heterocyclylenyl having 1-3 heteroatoms independently selected from nitrogen, oxygen, and sulfur. In some embodiments, -Cy^{L1}- is an optionally substituted 9-11 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 nitrogen heteroatoms. In some embodiments, -Cy^{L1}- is an optionally substituted 4,6-spiro heterocyclylenyl having 1-3 heteroatoms independently selected from nitrogen, oxygen, and sulfur. In some embodiments, -C^{L2}- is an optionally substituted

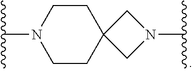 [SMILES: *N1CCC2(CC1)CN(*)C2]

In some embodiments, -Cy^{L2}- is an optionally substituted 10 membered saturated or partially unsaturated spiro heterocyclylenyl having 1-3 heteroatoms independently selected from nitrogen, oxygen, and sulfur. In some embodiments, -Cy^{L2}i- is an optionally substituted 10 membered saturated or partially unsaturated spiro heterocyclylenyl having 1-3 nitrogen heteroatoms. In some embodiments, -Cy^{L1}- is an optionally substituted 5,6-spiro heterocyclylenyl having 1-3 heteroatoms independently selected from nitrogen, oxygen, and sulfur.

In some embodiments, -Cy^{L2}- is an optionally substituted 11 membered saturated or partially unsaturated spiro heterocyclylenyl having 1-3 heteroatoms independently selected from nitrogen, oxygen, and sulfur. In some embodiments, -Cy^{L1}- is an optionally substituted 11 membered saturated or partially unsaturated spiro heterocyclylenyl having 1-3 nitrogen heteroatoms. In some embodiments, -Cy^{L1}- is an optionally substituted 6,6-spiro heterocyclylenyl having 1-3 heteroatoms independently selected from nitrogen, oxygen, and sulfur.

In some embodiments, -Cy^{L1}- is optionally substituted

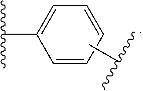

In some embodiments, -Cy^{L1}- is

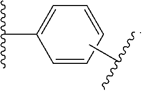

In some embodiments, -Cy^{L1}- is

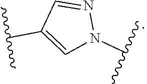 [SMILES: *c1cnn(*)c1]

In some embodiments, -Cy^{L1}- is

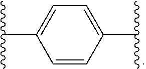 [SMILES: *c1ccc(*)cc1]

In some embodiments, -Cy^{L1}- is

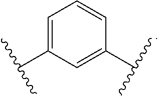 [SMILES: *c1cccc(*)c1]

In some embodiments, -Cy^{L1}- is

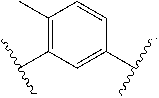 [SMILES: *c1ccc(C)c(*)c1]

In some embodiments, -Cy^{L1}- is

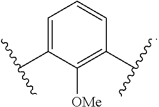 [SMILES: *c1cccc(*)c1OC]

In some embodiments, -Cy^{L1}- is

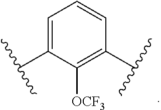 [SMILES: *c1cccc(*)c1OC(F)(F)F]

In some embodiments, -Cy^{L1}- is

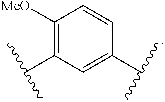 [SMILES: *c1ccc(OC)c(*)c1]

In some embodiments, -Cy^{L1}- is

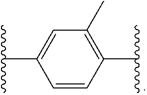 [SMILES: *c1ccc(*)c(C)c1]

In some embodiments, -Cy^{L1}- is

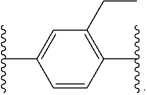 [SMILES: *c1ccc(*)c(CC)c1]

In some embodiments, -Cy^{L1}- is

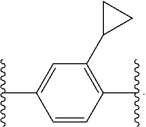 [SMILES: *c1ccc(*)c(C2CC2)c1]

In some embodiments, -Cy^{L1}- is

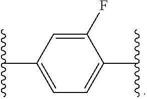 [SMILES: *c1ccc(*)c(F)c1]

In some embodiments, -Cy^{L1}- is

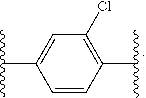 [SMILES: *c1ccc(*)c(Cl)c1]

In some embodiments, -Cy^{L1}- is

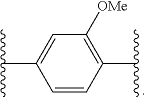 [SMILES: *c1ccc(*)c(OC)c1]

In some embodiments, -Cy^{L1}- is

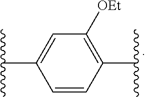 [SMILES: *c1ccc(*)c(OCC)c1]

In some embodiments, -Cy^{L1}- is

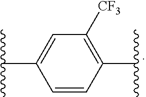 [SMILES: *c1ccc(*)c(C(F)(F)F)c1]

In some embodiments, -Cy^{L1}- is

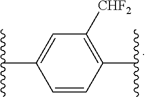 [SMILES: *c1ccc(*)c(C(F)F)c1]

In some embodiments, -Cy^{L1}- is

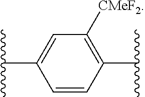

In some embodiments, -Cy^{L1}- is

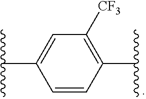 [SMILES: *c1ccc(*)c(C(F)(F)F)c1]

In some embodiments, -Cy^{L1}- is

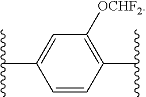 [SMILES: *c1ccc(*)c(OC(F)F)c1]

In some embodiments, -Cy^{L1}- is

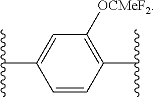

In some embodiments, -Cy^{L1}- is

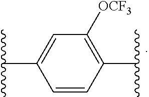 [SMILES: *c1ccc(*)c(OC(F)(F)F)c1]

In some embodiments, -Cy^{L1}- is

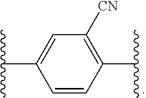 [SMILES: *c1ccc(*)c(C#N)c1]

In some embodiments, -Cy^{L1}- is

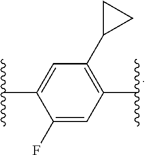 [SMILES: *c1cc(C2CC2)c(*)cc1F]

In some embodiments, -Cy^{L1}- is

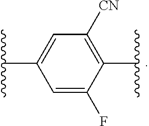 [SMILES: *c1cc(F)c(*)c(C#N)c1]

In some embodiments, -Cy^{L1}- is

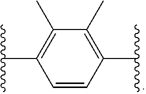 [SMILES: *c1ccc(*)c(C)c1C]

In some embodiments, -Cy^{L1}- is

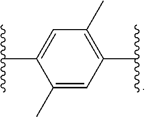 [SMILES: *c1cc(C)c(*)cc1C]

In some embodiments, -Cy^{L1}- is

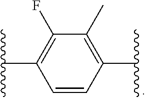 [SMILES: *c1ccc(*)c(F)c1C]

In some embodiments, -Cy^{L1}- is

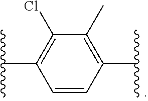 [SMILES: *c1ccc(*)c(Cl)c1C]

In some embodiments, -Cy^{L1}- is

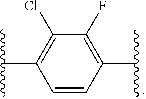 [SMILES: *c1ccc(*)c(Cl)c1F]

In some embodiments, -Cy^{L1}- is

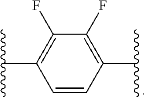 [SMILES: *c1ccc(*)c(F)c1F]

In some embodiments, -Cy^{L1}- is

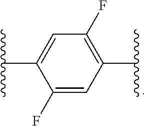 [SMILES: *c1cc(F)c(*)cc1F]

In some embodiments, -Cy^{L1}- is

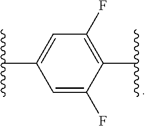 [SMILES: *c1cc(F)c(*)c(F)c1]

In some embodiments, -Cy^{L1}- is

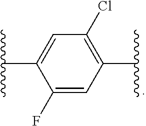 [SMILES: *c1cc(Cl)c(*)cc1F]

In some embodiments, -Cy^{L1}- is

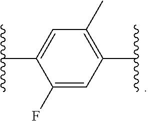 [SMILES: *c1cc(F)c(*)cc1C]

In some embodiments, -Cy^{L1}- is

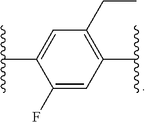 [SMILES: *c1cc(CC)c(*)cc1F]

In some embodiments, -Cy^{L1}- is

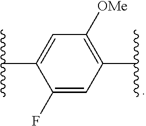 [SMILES: *c1cc(OC)c(*)cc1F]

In some embodiments, -Cy^{L1}- is

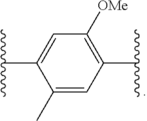 [SMILES: *c1cc(OC)c(*)cc1C]

In some embodiments, -Cy^{L1}- is

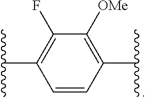 [SMILES: *c1ccc(*)c(OC)c1F]

In some embodiments, -Cy^{L1}- is

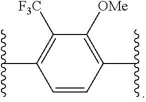 [SMILES: *c1ccc(*)c(C(F)(F)F)c1OC]

In some embodiments, -Cy^{L1}- is

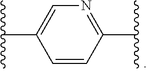 [SMILES: *c1ccc(*)nc1]

In some embodiments, -Cy^{L1}- is

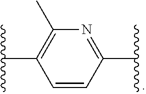 [SMILES: *c1ccc(*)c(C)n1]

In some embodiments, -Cy^{L1}- is

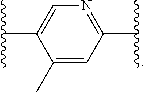 [SMILES: *c1cc(C)c(*)cn1]

In some embodiments, -Cy^{L1}- is

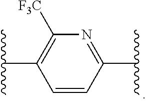 [SMILES: *c1ccc(*)c(C(F)(F)F)n1]

In some embodiments, -Cy^{L1}- is

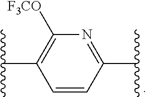 [SMILES: *c1ccc(*)c(OC(F)(F)F)n1]

In some embodiments, -Cy^{L1}- is

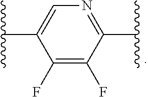 [SMILES: *c1cnc(*)c(F)c1F]

In some embodiments, -Cy^{L1}- is

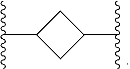 [SMILES: *C1CC(*)C1]

In some embodiments, -Cy^{L1}- is

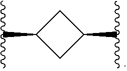 [SMILES: *[C@H]1C[C@@H](*)C1]

In some embodiments, -Cy^{L1}- is

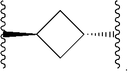 [SMILES: *[C@H]1C[C@H](*)C1]

In some embodiments, -Cy^{L1}- is

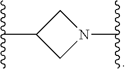 [SMILES: *C1CN(*)C1]

In some embodiments, -Cy^{L1}- is

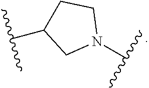 [SMILES: *C1CCN(*)C1]

In some embodiments, -Cy^{L1}- is

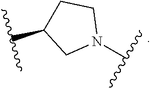 [SMILES: *[C@H]1CCN(*)C1]

In some embodiments, -Cy^{L1}- is

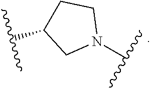 [SMILES: *[C@@H]1CCN(*)C1]

In some embodiments, -Cy^{L1}- is

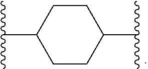 [SMILES: *C1CCC(*)CC1]

In some embodiments, -Cy^{L1}- is

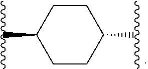 [SMILES: *[C@H]1CC[C@H](*)CC1]

In some embodiments, -Cy^{L1}- is

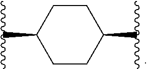 [SMILES: *[C@H]1CC[C@@H](*)CC1]

In some embodiments, -Cy^{L1}- is

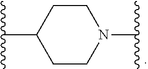 [SMILES: *C1CCN(*)CC1]

In some embodiments, -Cy^{L1}- is

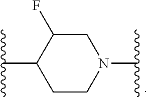 [SMILES: *C1CCN(*)CC1F]

In some embodiments, -Cy^{L1}- is

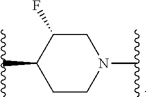 [SMILES: *[C@@H]1CCN(*)C[C@H]1F]

In some embodiments, -Cy^{L1}- is

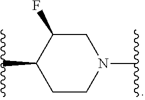 [SMILES: *[C@@H]1CCN(*)C[C@@H]1F]

In some embodiments, -Cy^{L1}- is

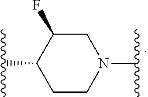 [SMILES: *[C@H]1CCN(*)C[C@@H]1F]

In some embodiments, -Cy^{L1}- is

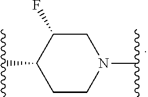 [SMILES: *[C@H]1CCN(*)C[C@H]1F]

In some embodiments, -Cy^{L1}- is

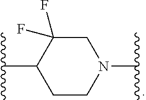 [SMILES: *C1CCN(*)CC1(F)F]

In some embodiments, -Cy^{L1}- is

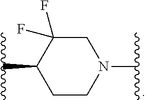 [SMILES: *[C@@H]1CCN(*)CC1(F)F]

In some embodiments, -Cy^{L1}- is

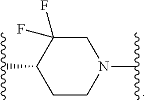 [SMILES: *[C@H]1CCN(*)CC1(F)F]

In some embodiments, -Cy^{L1}- is

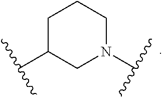 [SMILES: *C1CCCN(*)C1]

In some embodiments, -Cy^{L1}- is

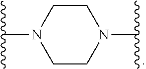 [SMILES: *N1CCN(*)CC1]

In some embodiments, -Cy^{L1}- is

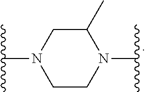 [SMILES: *N1CCN(*)C(C)C1]

In some embodiments, -Cy^{L1}- is

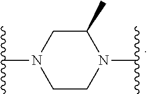 [SMILES: *N1CCN(*)[C@H](C)C1]

In some embodiments, -Cy^{L1}- is

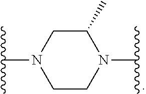 [SMILES: *N1CCN(*)[C@@H](C)C1]

In some embodiments, -Cy^{L1}- is

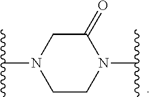 [SMILES: *N1CCN(*)C(=O)C1]

In some embodiments, -Cy^{L1}- is

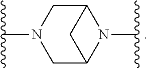 [SMILES: *N1CC2CC(C1)N2*]

In some embodiments, -Cy^{L1}- is

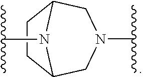 [SMILES: *N1CC2CCC(C1)N2*]

In some embodiments, -Cy^{L1}- is

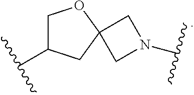 [SMILES: *C1COC2(C1)CN(*)C2]

In some embodiments, -Cy^{L1}- is

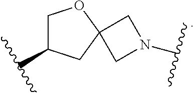 [SMILES: *[C@H]1COC2(C1)CN(*)C2]

In some embodiments, -Cy^{L1}- is

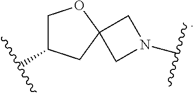 [SMILES: *[C@@H]1COC2(C1)CN(*)C2]

In some embodiments, -Cy^{L1}- is

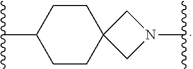 [SMILES: *C1CCC2(CC1)CN(*)C2]

In some embodiments, -Cy^{L1}- is

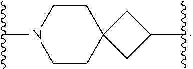 [SMILES: *C1CC2(CCN(*)CC2)C1]

In some embodiments, -Cy^{L1}- is

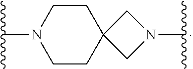 [SMILES: *N1CCC2(CC1)CN(*)C2]

In some embodiments, -Cy^{L1}- is

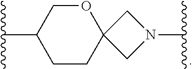 [SMILES: *C1CCC2(CN(*)C2)OC1]

In some embodiments, -Cy^{L1}- is

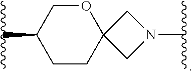 [SMILES: *[C@@H]1CCC2(CN(*)C2)OC1]

In some embodiments, -Cy^{L1}- is

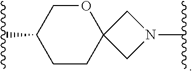 [SMILES: *[C@H]1CCC2(CN(*)C2)OC1]

In some embodiments, -Cy^{L1}- is

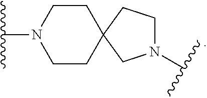 [SMILES: *N1CCC2(CC1)CCN(*)C2]

In some embodiments, -Cy^{L1}- is

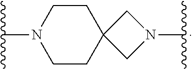 [SMILES: *N1CCC2(CC1)CN(*)C2]

In some embodiments, -Cy^{L1}- is

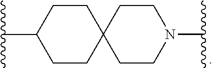 [SMILES: *C1CCC2(CC1)CCN(*)CC2]

In some embodiments, -Cy^{L1}- is

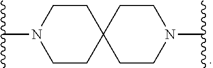 [SMILES: *N1CCC2(CC1)CCN(*)CC2]

In some embodiments, -Cy^{L1}- is

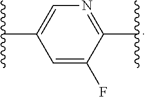 [SMILES: *c1cnc(*)c(F)c1]

In some embodiments, -Cy^{L1}- is an optionally substituted 8-10 membered bicyclic arylenyl. In some embodiments, -Cy^{L2}- is an optionally substituted 3-7 membered saturated or partially unsaturated carbocyclylenyl. In some embodiments, -Cy^{L2}- is an optionally substituted 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is an optionally substituted a 4-7 membered saturated or partially unsaturated heterocyclylenyl having 1-2 heteroatoms independently selected from nitrogen, oxygen, and sulfur. In some embodiments, -Cy^{L2}- is an optionally substituted 6-11 membered saturated or partially unsaturated spiro heterocyclylenyl having 1-3 heteroatoms independently selected from nitrogen, oxygen, and sulfur. In some embodiments, -Cy^{L2}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L2}- is an optionally substituted 5-6 membered heteroarylenyl having 1-4 heteroatoms independently selected from nitrogen, oxygen. In some embodiments, -Cy^{L2}- is an optionally substituted 8-10 membered bicyclic heteroarylenyl having 1-5 heteroatoms independently selected from nitrogen, oxygen, and sulfur.

In some embodiments, each -Cy^{L2}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an 8-10 membered bicyclic arylenyl. In some embodiments, -Cy^{L2}- is a 3-7 membered saturated or partially unsaturated carbocyclylenyl. In some embodiments, -Cy^{L2}- is a 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is a 4-7 membered saturated or partially unsaturated heterocyclylenyl having 1-2 heteroatoms independently selected from nitrogen, oxygen, and sulfur. In some embodiments, -Cy^{L2}- is a 6-11 membered saturated or partially unsaturated spiro heterocyclylenyl having 1-3 heteroatoms independently selected from nitrogen, oxygen, and sulfur. In some embodiments, -Cy^{L2}- is an 8-10 membered bicyclic saturated or partially unsaturated heterocyclylenyl having 1-2 heteroatoms independently selected from nitrogen, oxygen, and sulfur. In some embodiments, -Cy^{L2}- is a 5-6 membered heteroarylenyl having 1-4 heteroatoms independently selected from nitrogen, oxygen. In some embodiments, -Cy^{L2}- is an 8-10 membered bicyclic heteroarylenyl having 1-5 heteroatoms independently selected from nitrogen, oxygen, and sulfur.

In some embodiments, -Cy^{L2}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L2}- is an optionally substituted phenylenyl. In some embodiments, -Cy^{L2}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is phenylenyl, optionally substituted with halogen (e.g., fluoro or chloro) or C₁₋₆ aliphatic.

In some embodiments, -Cy^{L2}- is an optionally substituted 5-6 membered saturated or partially unsaturated heterocyclylenyl having 1-2 heteroatoms independently selected from nitrogen, oxygen, and sulfur. In some embodiments, -Cy^{L2}- is an optionally substituted 5 membered saturated or partially unsaturated heterocyclylenyl having 1-2 heteroatoms independently selected from nitrogen, oxygen, and sulfur. In some embodiments, -Cy^{L2}- is an optionally substituted pyrrolidinylenyl. In some embodiments, -Cy^{L2}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyrrolidinylenyl, optionally substituted with halogen (e.g., fluoro or chloro) or C₆ aliphatic. In some embodiments, -Cy^{L2}- is an optionally substituted 6 membered saturated or partially unsaturated heterocyclylenyl having 1-2 heteroatoms independently selected from nitrogen, oxygen, and sulfur. In some embodiments, -Cy^{L2}- is an optionally substituted piperadinylenyl. In some embodiments, -Cy^{L2}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is piperadinylenyl, optionally substituted with halogen (e.g., fluoro or chloro) or C₁₋₆ aliphatic. In some embodiments, -Cy^{L2}- is an optionally substituted piperazinylenyl. In some embodiments, -Cy^{L2}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is piperazinylenyl, optionally substituted with halogen (e.g., fluoro or chloro) or C₁₋₆ aliphatic.

In some embodiments, -Cy^{L1}- is an optionally substituted 5 membered heteroarylenyl having 1-4 heteroatoms independently selected from nitrogen, oxygen. In some embodiments, -Cy^{L2}- is an optionally substituted pyrrolylenyl, pyrazolylenyl, imidazolylenyl, or triazolylenyl. In some embodiments, -Cy^{L2}- is pyrrolylenyl, pyrazolylenyl, imidazolylenyl, or triazolylene, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L2}- is an optionally substituted pyrazolylenyl. In some embodiments, -Cy^{L2}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyrazolylenyl, optionally substituted with halogen (e.g., fluoro or chloro) or C₁₋₆ aliphatic. In some embodiments, -Cy^{L2}- is an optionally substituted imidazolylenyl. In some embodiments, -Cy^{L2}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L2}- is imidazolylenyl, optionally substituted with halogen (e.g., fluoro or chloro) or C₁₋₆ aliphatic.

In some embodiments, -Cy^{L2}- is an optionally substituted 6 membered heteroarylenyl having 1-4 heteroatoms independently selected from nitrogen, oxygen. In some embodiments, -Cy^{L2}- is an optionally substituted pyridinylenyl, pyridazinylenyl, pyrimidinylenyl, pyrazinylenyl, or triazinylenyl. In some embodiments, -Cy^{L2}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₁₀ aliphatic. In some embodiments, -Cy^{L2}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L2}- is an optionally substituted pyridinylenyl. In some embodiments, -Cy^{L2}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridinylenyl, optionally substituted with halogen (e.g., fluoro or chloro) or C₁₋₆ aliphatic. In some embodiments, -Cy^{L2}- is an optionally substituted pyridazinylenyl. In some embodiments, -Cy^{L2}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridazinylenyl, optionally substituted with halogen (e.g., fluoro or chloro) or C₁₋₆ aliphatic. In some embodiments, -Cy^{L2}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L2}- is an optionally substituted pyridinonylenyl. In some embodiments, -Cy^{L2}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridinonylenyl, optionally substituted with halogen (e.g., fluoro or chloro) or C₁₋₆ aliphatic.

In some embodiments, -Cy^{L2}- is an optionally substituted cyclopropyl. In some embodiments, -Cy^{L2}- is an optionally substituted 5 membered saturated or partially unsaturated carbocyclylenyl. In some embodiments, -Cy- is an optionally substituted cyclopentanylenyl or cyclopentenylenyl. In some embodiments, -Cy^{L2}- is an optionally substituted 6 membered saturated or partially unsaturated carbocyclylenyl. In some embodiments, -Cy^{L2}- is an optionally substituted cyclohexanylenyl or cyclohexenylenyl. In some embodiments, -Cy^{L2}- is an optionally substituted naphthalenylenyl.

In some embodiments, -Cy^{L2}- is an optionally substituted 9 membered bicyclic heteroarylenyl having 1-5 heteroatoms independently selected from nitrogen, oxygen, and sulfur. In some embodiments, -Cy^{L2}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L2}- is an optionally substituted indolylenyl. In some embodiments, -Cy^{L2}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L2}- is an optionally substituted 8-11 membered saturated or partially unsaturated spiro heterocyclylenyl having 1-3 heteroatoms independently selected from nitrogen, oxygen, and sulfur. In some embodiments, -Cy^{L2}- is an optionally substituted 9-11 membered saturated or partially unsaturated spiro heterocyclylenyl having 1-3 heteroatoms independently selected from nitrogen, oxygen, and sulfur. In some embodiments, -Cy^{L2}- is an optionally substituted 9 membered saturated or partially unsaturated spiro heterocyclylenyl having 1-3 heteroatoms independently selected from nitrogen, oxygen, and sulfur. In some embodiments, -Cy^{L2}- is an optionally substituted 9 membered saturated or partially unsaturated spiro heterocyclylenyl having 1-3 nitrogen heteroatoms. In some embodiments, -Cy^{L2}- is an optionally substituted 4,6-spiro heterocyclylenyl having 1-3 heteroatoms independently selected from nitrogen, oxygen, and sulfur. In some embodiments, -Cy^{L2}- is an optionally substituted

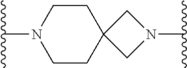 [SMILES: *N1CCC2(CC1)CN(*)C2]

In some embodiments, -Cy^{L2}- is an optionally substituted 10 membered saturated or partially unsaturated spiro heterocyclylenyl having 1-3 heteroatoms independently selected from nitrogen, oxygen, and sulfur. In some embodiments, -Cy^{L2}- is an optionally substituted 10 membered saturated or partially unsaturated spiro heterocyclylenyl having 1-3 nitrogen heteroatoms. In some embodiments, -Cy^{L2}- is an optionally substituted 5,6-spiro heterocyclylenyl having 1-3 heteroatoms independently selected from nitrogen, oxygen, and sulfur.

In some embodiments, -Cy^{L2}- is an optionally substituted 11 membered saturated or partially unsaturated spiro heterocyclylenyl having 1-3 heteroatoms independently selected from nitrogen, oxygen, and sulfur. In some embodiments, -Cy^{L1}- is an optionally substituted 11 membered saturated or partially unsaturated spiro heterocyclylenyl having 1-3 nitrogen heteroatoms. In some embodiments, -Cy^{L2}- is an optionally substituted 6,6-spiro heterocyclylenyl having 1-3 heteroatoms independently selected from nitrogen, oxygen, and sulfur.

In some embodiments, -Cy^{L2}- is optionally substituted

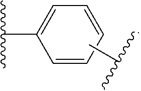

In some embodiments, -Cy^{L2}- is

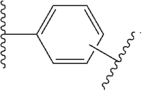

In some embodiments, -Cy^{L2}- is

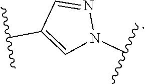 [SMILES: *c1cnn(*)c1]

In some embodiments, -Cy^{L2}- is

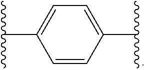 [SMILES: *c1ccc(*)cc1]

In some embodiments, -Cy^{L2}- is

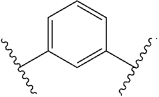 [SMILES: *c1cccc(*)c1]

In some embodiments, -Cy^{L2}- is

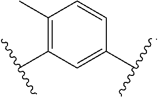 [SMILES: *c1ccc(C)c(*)c1]

In some embodiments, -Cy^{L2}- is

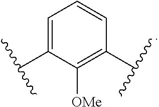 [SMILES: *c1cccc(*)c1OC]

In some embodiments, -Cy^{L2}- is

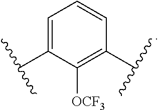 [SMILES: *c1cccc(*)c1OC(F)(F)F]

In some embodiments, -Cy^{L2}- is

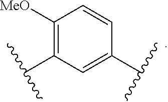 [SMILES: *c1ccc(OC)c(*)c1]

In some embodiments, -Cy^{L2}- is

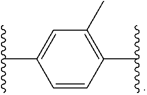 [SMILES: *c1ccc(*)c(C)c1]

In some embodiments, -Cy^{L2}- is

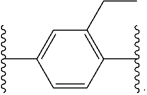 [SMILES: *c1ccc(*)c(CC)c1]

In some embodiments, -Cy^{L2}- is

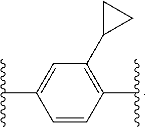 [SMILES: *c1ccc(*)c(C2CC2)c1]

In some embodiments, -Cy^{L2}- is

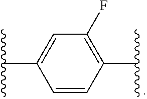 [SMILES: *c1ccc(*)c(F)c1]

In some embodiments, -Cy^{L2}- is

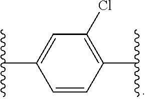 [SMILES: *c1ccc(*)c(Cl)c1]

In some embodiments, -Cy^{L2}- is

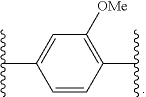 [SMILES: *c1ccc(*)c(OC)c1]

In some embodiments, -Cy^{L2}- is

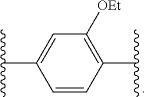 [SMILES: *c1ccc(*)c(OCC)c1]

In some embodiments, -Cy^{L2}- is

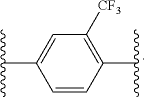 [SMILES: *c1ccc(*)c(C(F)(F)F)c1]

In some embodiments, -Cy^{L2}- is

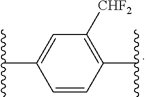 [SMILES: *c1ccc(*)c(C(F)F)c1]

In some embodiments, -Cy^{L2}- is

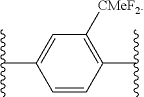

In some embodiments, -Cy^{L2}- is

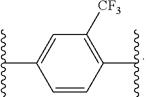 [SMILES: *c1ccc(*)c(C(F)(F)F)c1]

In some embodiments, -Cy^{L2}- is

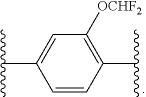 [SMILES: *c1ccc(*)c(OC(F)F)c1]

In some embodiments, -Cy^{L2}- is

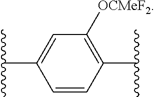

In some embodiments, -Cy^{L2}- is

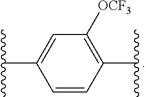 [SMILES: *c1ccc(*)c(OC(F)(F)F)c1]

In some embodiments, -Cy^{L2}- is

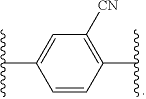 [SMILES: *c1ccc(*)c(C#N)c1]

In some embodiments, -Cy^{L2}- is

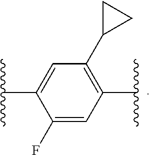 [SMILES: *c1cc(C2CC2)c(*)cc1F]

In some embodiments, -Cy^{L2}- is

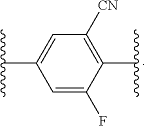 [SMILES: *c1cc(F)c(*)c(C#N)c1]

In some embodiments, -Cy^{L2}- is

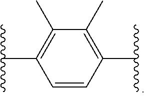 [SMILES: *c1ccc(*)c(C)c1C]

In some embodiments, -Cy^{L2}- is

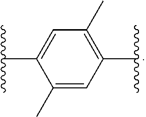 [SMILES: *c1cc(C)c(*)cc1C]

In some embodiments, -Cy^{L2}- is

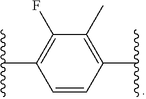 [SMILES: *c1ccc(*)c(F)c1C]

In some embodiments, -Cy^{L2}- is

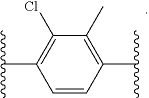 [SMILES: *c1ccc(*)c(Cl)c1C]

In some embodiments, -Cy^{L2}- is

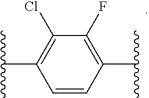 [SMILES: *c1ccc(*)c(Cl)c1F]

In some embodiments, -Cy^{L2}- is

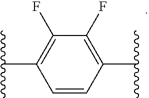 [SMILES: *c1ccc(*)c(F)c1F]

In some embodiments, -Cy^{L2}- is

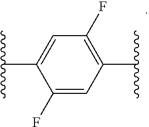 [SMILES: *c1cc(F)c(*)cc1F]

In some embodiments, -Cy^{L2}- is

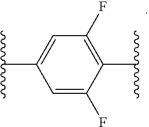 [SMILES: *c1cc(F)c(*)c(F)c1]

In some embodiments, -Cy^{L2}- is

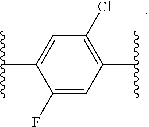 [SMILES: *c1cc(Cl)c(*)cc1F]

In some embodiments, -Cy^{L2}- is

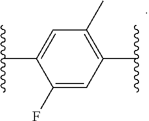 [SMILES: *c1cc(F)c(*)cc1C]

In some embodiments, -Cy^{L2}- is

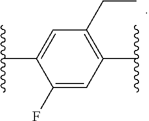 [SMILES: *c1cc(CC)c(*)cc1F]

In some embodiments, -Cy^{L2}- is

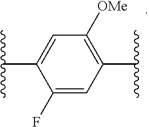 [SMILES: *c1cc(OC)c(*)cc1F]

In some embodiments, -Cy^{L2}- is

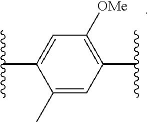 [SMILES: *c1cc(OC)c(*)cc1C]

In some embodiments, -Cy^{L2}- is

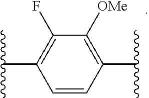 [SMILES: *c1ccc(*)c(OC)c1F]

In some embodiments, -Cy^{L2}- is

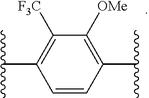 [SMILES: *c1ccc(*)c(C(F)(F)F)c1OC]

In some embodiments, -Cy^{L2}- is

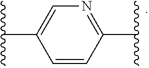 [SMILES: *c1ccc(*)nc1]

In some embodiments, -Cy^{L2}- is

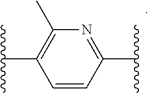 [SMILES: *c1ccc(*)c(C)n1]

In some embodiments, -Cy^{L2}- is

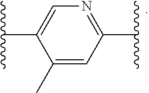 [SMILES: *c1cc(C)c(*)cn1]

In some embodiments, -Cy^{L2}- is

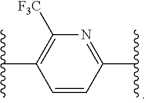 [SMILES: *c1ccc(*)c(C(F)(F)F)n1]

In some embodiments, -Cy^{L2}- is

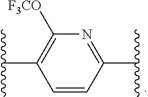 [SMILES: *c1ccc(*)c(OC(F)(F)F)n1]

In some embodiments, -Cy^{L2}- is

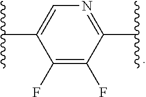 [SMILES: *c1cnc(*)c(F)c1F]

In some embodiments, -Cy^{L2}- is

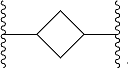 [SMILES: *C1CC(*)C1]

In some embodiments, -Cy^{L2}- is

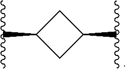 [SMILES: *[C@H]1C[C@@H](*)C1]

In some embodiments, -Cy^{L2}- is

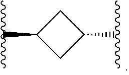 [SMILES: *[C@H]1C[C@H](*)C1]

In some embodiments, -Cy^{L2}- is

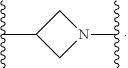 [SMILES: *C1CN(*)C1]

In some embodiments, -Cy^{L2}- is

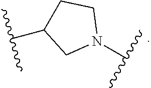 [SMILES: *C1CCN(*)C1]

In some embodiments, -Cy^{L2}- is

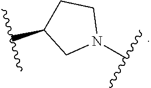 [SMILES: *[C@H]1CCN(*)C1]

In some embodiments, -Cy^{L2}- is

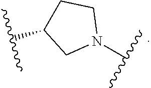 [SMILES: *[C@@H]1CCN(*)C1]

In some embodiments, -Cy^{L2}- is

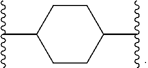 [SMILES: *C1CCC(*)CC1]

In some embodiments, -Cy^{L2}- is

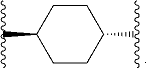 [SMILES: *[C@H]1CC[C@H](*)CC1]

In some embodiments, -Cy^{L2}- is

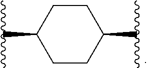 [SMILES: *[C@H]1CC[C@@H](*)CC1]

In some embodiments, -Cy^{L2}- is

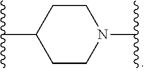 [SMILES: *C1CCN(*)CC1]

In some embodiments, -Cy^{L2}- is

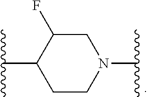 [SMILES: *C1CCN(*)CC1F]

In some embodiments, -Cy^{L2}- is

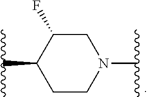 [SMILES: *[C@@H]1CCN(*)C[C@H]1F]

In some

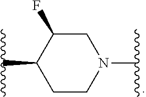 [SMILES: *[C@@H]1CCN(*)C[C@@H]1F]

In some embodiments, -Cy^{L2}- is

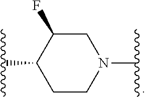 [SMILES: *[C@H]1CCN(*)C[C@@H]1F]

In some embodiments, -Cy^{L2}- is

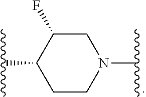 [SMILES: *[C@H]1CCN(*)C[C@H]1F]

In some embodiments, -Cy^{L2}- is

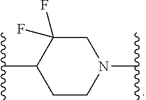 [SMILES: *C1CCN(*)CC1(F)F]

In some embodiments, -Cy^{L2}- is

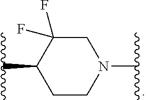 [SMILES: *[C@@H]1CCN(*)CC1(F)F]

In some embodiments, -Cy^{L2}- is

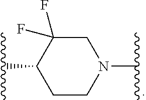 [SMILES: *[C@H]1CCN(*)CC1(F)F]

In some embodiments, -Cy^{L2}- is

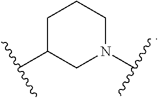 [SMILES: *C1CCCN(*)C1 ; "."]

In some embodiments, -Cy^{L2}- is

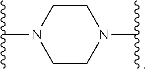 [SMILES: *N1CCN(*)CC1 ; "."]

In some embodiments, -Cy^{L2}- is

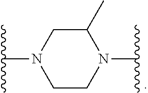 [SMILES: *N1CCN(*)C(C)C1]

In some embodiments, -Cy^{L2}- is

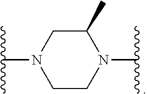 [SMILES: *N1CCN(*)[C@H](C)C1 ; "."]

In some embodiments, -Cy^{L2}- is

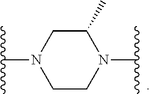 [SMILES: *N1CCN(*)[C@@H](C)C1]

In some embodiments, -Cy^{L2}- is

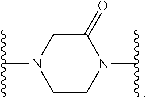 [SMILES: *N1CCN(*)C(=O)C1 ; "."]

In some embodiments, -Cy^{L2}- is

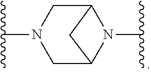 [SMILES: *N1CC2CC(C1)N2*]

In some embodiments, -Cy^{L2}- is

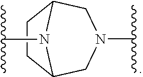 [SMILES: *N1CC2CCC(C1)N2*]

In some embodiments, -Cy^{L2}- is

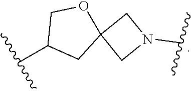 [SMILES: *C1COC2(C1)CN(*)C2]

In some embodiments, -Cy^{L2}- is

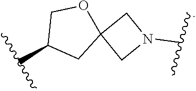 [SMILES: *[C@H]1COC2(C1)CN(*)C2]

In some embodiments, -Cy^{L2}- is

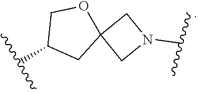 [SMILES: *[C@@H]1COC2(C1)CN(*)C2]

In some embodiments, -Cy^{L2}- is

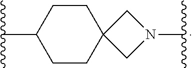 [SMILES: *C1CCC2(CC1)CN(*)C2]

In some embodiments, -Cy^{L2}- is

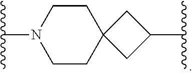 [SMILES: *C1CC2(CCN(*)CC2)C1]

In some embodiments, -Cy^{L2}- is

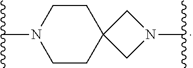 [SMILES: *N1CCC2(CC1)CN(*)C2]

In some embodiments, -Cy^{L2}- is

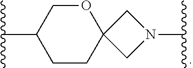 [SMILES: *C1CCC2(CN(*)C2)OC1]

In some embodiments, -Cy^{L2}- is

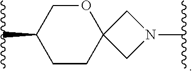 [SMILES: *[C@@H]1CCC2(CN(*)C2)OC1]

In some embodiments, -Cy^{L2}- is

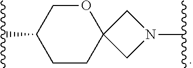 [SMILES: *[C@H]1CCC2(CN(*)C2)OC1]

In some embodiments, -Cy^{L2}- is

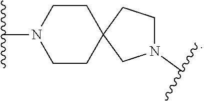 [SMILES: *N1CCC2(CC1)CCN(*)C2]

In some embodiments, -Cy^{L2}- is

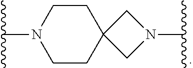 [SMILES: *N1CCC2(CC1)CN(*)C2]

In some embodiments, -Cy^{L2}- is

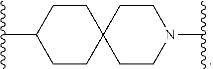 [SMILES: *C1CCC2(CC1)CCN(*)CC2]

In some embodiments, -Cy^{L2}- is

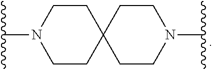 [SMILES: *N1CCC2(CC1)CCN(*)CC2]

In some embodiments, -Cy^{L2}- is

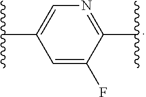 [SMILES: *c1cnc(*)c(F)c1]

In some embodiments, -Cy^{L1}- is an optionally substituted 8-10 membered bicyclic arylenyl. In some embodiments, -Cy^{L3}- is an optionally substituted 3-7 membered saturated or partially unsaturated carbocyclylenyl. In some embodiments, -Cy^{L3}- is an optionally substituted 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3}- is an optionally substituted a 4-7 membered saturated or partially unsaturated heterocyclylenyl having 1-2 heteroatoms independently selected from nitrogen, oxygen, and sulfur. In some embodiments, -Cy^{L3}- is an optionally substituted 6-11 membered saturated or partially unsaturated spiro heterocyclylenyl having 1-3 heteroatoms independently selected from nitrogen, oxygen, and sulfur. In some embodiments, -Cy^{L3}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L3}- is an optionally substituted 5-6 membered heteroarylenyl having 1-4 heteroatoms independently selected from nitrogen, oxygen. In some embodiments, -Cy^{L3}- is an optionally substituted 8-10 membered bicyclic heteroarylenyl having 1-5 heteroatoms independently selected from nitrogen, oxygen, and sulfur.

In some embodiments, each -Cy^{L3}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an 8-10 membered bicyclic arylenyl. In some embodiments, -Cy^{L3}- is a 3-7 membered saturated or partially unsaturated carbocyclylenyl. In some embodiments, -Cy^{L3}- is a 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3} is a 4-7 membered saturated or partially unsaturated heterocyclylenyl having 1-2 heteroatoms independently selected from nitrogen, oxygen, and sulfur. In some embodiments, -Cy^{L3}- is a 6-11 membered saturated or partially unsaturated spiro heterocyclylenyl having 1-3 heteroatoms independently selected from nitrogen, oxygen, and sulfur. In some embodiments, -Cy^{L3}- is an 8-10 membered bicyclic saturated or partially unsaturated heterocyclylenyl having 1-2 heteroatoms independently selected from nitrogen, oxygen, and sulfur. In some embodiments, -Cy^{L3}- is a 5-6 membered heteroarylenyl having 1-4 heteroatoms independently selected from nitrogen, oxygen. In some embodiments, -Cy^{L3}- is an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L3}- is an optionally substituted phenylenyl. In some embodiments, -Cy^{L3}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is phenylenyl, optionally substituted with halogen (e.g., fluoro or chloro) or C₁₋₆ aliphatic.

In some embodiments, -Cy^{L3}- is an optionally substituted 5-6 membered saturated or partially unsaturated heterocyclylenyl having 1-2 heteroatoms independently selected from nitrogen, oxygen, and sulfur. In some embodiments, -Cy^{L3}- is an optionally substituted 5 membered saturated or partially unsaturated heterocyclylenyl having 1-2 heteroatoms independently selected from nitrogen, oxygen, and sulfur. In some embodiments, -Cy^{L3}- is an optionally substituted pyrrolidinylenyl. In some embodiments, -Cy^{L3}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyrrolidinylenyl, optionally substituted with halogen (e.g., fluoro or chloro) or C₁₋₆ aliphatic. In some embodiments, -Cy^{L3}- is an optionally substituted 6 membered saturated or partially unsaturated heterocyclylenyl having 1-2 heteroatoms independently selected from nitrogen, oxygen, and sulfur. In some embodiments, -Cy^{L3}- is an optionally substituted piperadinylenyl. In some embodiments, -Cy^{L3}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is piperadinylenyl, optionally substituted with halogen (e.g., fluoro or chloro) or C₁₋₆ aliphatic. In some embodiments, -Cy^{L2}- is an optionally substituted piperazinylenyl. In some embodiments, -Cy^{L3}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is piperazinylenyl, optionally substituted with halogen (e.g., fluoro or chloro) or C₁₋₆ aliphatic.

In some embodiments, -Cy^{L3}- is an optionally substituted 5 membered heteroarylenyl having 1-4 heteroatoms independently selected from nitrogen, oxygen. In some embodiments, -Cy^{L3}- is an optionally substituted pyrrolylenyl, pyrazolylenyl, imidazolylenyl, or triazolylenyl. In some embodiments, -Cy^{L3}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L3}- is an optionally substituted pyrazolylenyl. In some embodiments, -Cy^{L3}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyrazolylenyl, optionally substituted with halogen (e.g., fluoro or chloro) or C₁₋₆ aliphatic. In some embodiments, -Cy^{L1}- is an optionally substituted imidazolylenyl. In some embodiments, -Cy^{L3}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L3}- is imidazolylenyl, optionally substituted with halogen (e.g., fluoro or chloro) or C₁₋₆ aliphatic.

In some embodiments, -Cy^{L3}- is an optionally substituted 6 membered heteroarylenyl having 1-4 heteroatoms independently selected from nitrogen, oxygen. In some embodiments, -Cy^{L3}- is an optionally substituted pyridinylenyl, pyridazinylenyl, pyrimidinylenyl, pyrazinylenyl, or triazinylenyl. In some embodiments, -Cy^{L3}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L3}- is an optionally substituted pyridinylenyl. In some embodiments, -Cy^{L3}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinylenyl, optionally substituted with halogen (e.g., fluoro or chloro) or C₆ aliphatic. In some embodiments, -Cy^{L3}- is an optionally substituted pyridazinylenyl. In some embodiments, -Cy^{L3}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L3}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, pyridazinonylenyl, pynmidinonylenyl, pyrazinonylenyl, or triazinonylenyl, optionally substituted with halogen (e.g., fluoro or chloro) or C₁₋₆ aliphatic. In some embodiments, -Cy^{L3}- is an optionally substituted pyridinonylenyl. In some embodiments, -Cy^{L3}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinonylenyl, optionally substituted with halogen (e.g., fluoro or chloro) or C₁₋₆ aliphatic.

In some embodiments, -Cy^{L1}- is an optionally substituted cyclopropyl. In some embodiments, -Cy^{L3}- is an optionally substituted 5 membered saturated or partially unsaturated carbocyclylenyl. In some embodiments, -Cy^{L3}- is an optionally substituted cyclopentanylenyl or cyclopentenylenyl. In some embodiments, -Cy^{L3}- is an optionally substituted 6 membered saturated or partially unsaturated carbocyclylenyl. In some embodiments, -Cy^{L3}- is an optionally substituted cyclohexanylenyl or cyclohexenylenyl. In some embodiments, -Cy^{L3}- is an optionally substituted naphthalenylenyl.

In some embodiments, -Cy^{L3}- is an optionally substituted 9 membered bicyclic heteroarylenyl having 1-5 heteroatoms independently selected from nitrogen, oxygen, and sulfur. In some embodiments, -Cy^{L3}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L3}- is an optionally substituted indolylenyl. In some embodiments, -Cy^{L3}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L1}- is an optionally substituted 8-11 membered saturated or partially unsaturated spiro heterocyclylenyl having 1-3 heteroatoms independently selected from nitrogen, oxygen, and sulfur. In some embodiments, -Cy^{L3}- is an optionally substituted 9-11 membered saturated or partially unsaturated spiro heterocyclylenyl having 1-3 heteroatoms independently selected from nitrogen, oxygen, and sulfur. In some embodiments, -Cy^{L3}- is an optionally substituted 9 membered saturated or partially unsaturated spiro heterocyclylenyl having 1-3 heteroatoms independently selected from nitrogen, oxygen, and sulfur. In some embodiments, -Cy^{L3}- is an optionally substituted 9 membered saturated or partially unsaturated spiro heterocyclylenyl having 1-3 nitrogen heteroatoms. In some embodiments, -Cy^{L3}- is an optionally substituted 4,6-spiro heterocyclylenyl having 1-3 heteroatoms independently selected from nitrogen, oxygen, and sulfur. In some embodiments, -Cy^{L3}- is an optionally substituted

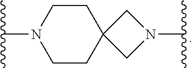 [SMILES: *N1CCC2(CC1)CN(*)C2]

In some embodiments, -Cy^{L3}- is an optionally substituted 10 membered saturated or partially unsaturated spiro heterocyclylenyl having 1-3 heteroatoms independently selected from nitrogen, oxygen, and sulfur. In some embodiments, -Cy^{L1}- is an optionally substituted 10 membered saturated or partially unsaturated spiro heterocyclylenyl having 1-3 nitrogen heteroatoms. In some embodiments, -Cy^{L3}- is an optionally substituted 5,6-spiro heterocyclylenyl having 1-3 heteroatoms independently selected from nitrogen, oxygen, and sulfur.

In some embodiments, -Cy^{L3}- is an optionally substituted 11 membered saturated or partially unsaturated spiro heterocyclylenyl having 1-3 heteroatoms independently selected from nitrogen, oxygen, and sulfur. In some embodiments, -Cy^{L3}- is an optionally substituted 11 membered saturated or partially unsaturated spiro heterocyclylenyl having 1-3 nitrogen heteroatoms. In some embodiments, -Cy^{L3}- is an optionally substituted 6,6-spiro heterocyclylenyl having 1-3 heteroatoms independently selected from nitrogen, oxygen, and sulfur.

In some embodiments, -Cy^{L3}- is optionally substituted

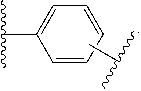

In some embodiments, -Cy^{L3}- is

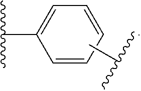

In some embodiments, -Cy^{L3}- is

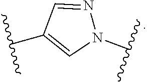 [SMILES: *c1cnn(*)c1]

In some embodiments, -Cy^{L3}- is

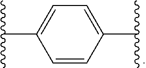 [SMILES: *c1ccc(*)cc1]

In some embodiments, -Cy^{L3}- is

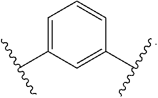 [SMILES: *c1cccc(*)c1]

In some embodiments, -Cy^{L3}- is

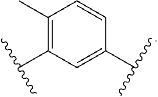 [SMILES: *c1ccc(C)c(*)c1]

In some embodiments, -Cy^{L3}- is

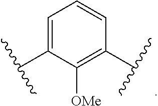 [SMILES: *c1cccc(*)c1OC]

In some embodiments, -Cy^{L3}- is

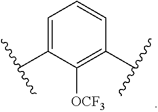 [SMILES: *c1cccc(*)c1OC(F)(F)F]

In some embodiments, -Cy^{L3}- is

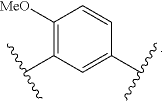 [SMILES: *c1ccc(OC)c(*)c1]

In some embodiments, -Cy^{L3}- is

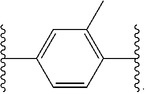 [SMILES: *c1ccc(*)c(C)c1]

In some embodiments, -Cy^{L3}- is

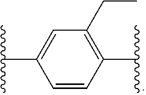 [SMILES: *c1ccc(*)c(CC)c1]

In some embodiments, -Cy^{L3}- is

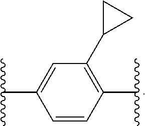 [SMILES: *c1ccc(*)c(C2CC2)c1]

In some embodiments, -Cy^{L3}- is

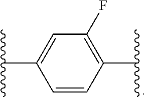 [SMILES: *c1ccc(*)c(F)c1]

In some embodiments, -Cy^{L3}- is

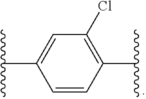 [SMILES: *c1ccc(*)c(Cl)c1]

In some embodiments, -Cy^{L3}- is

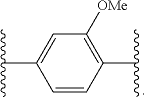 [SMILES: *c1ccc(*)c(OC)c1]

In some embodiments, -Cy^{L3}- is

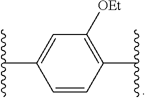 [SMILES: *c1ccc(*)c(OCC)c1]

In some embodiments, -Cy^{L3}- is

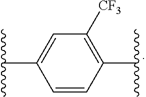 [SMILES: *c1ccc(*)c(C(F)(F)F)c1]

In some embodiments, -Cy^{L3}- is

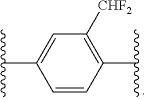 [SMILES: *c1ccc(*)c(C(F)F)c1]

In some embodiments, -Cy^{L3}- is

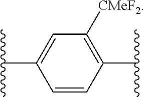

In some embodiments, -Cy^{L3}- is

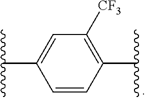 [SMILES: *c1ccc(*)c(C(F)(F)F)c1]

In some embodiments, -Cy^{L3}- is

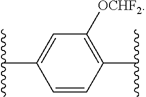 [SMILES: *c1ccc(*)c(OC(F)F)c1]

In some embodiments, -Cy^{L3}- is

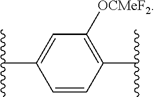

In some embodiments, -Cy^{L3}- is

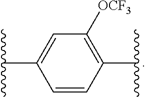 [SMILES: *c1ccc(*)c(OC(F)(F)F)c1]

In some embodiments, -Cy^{L3}- is

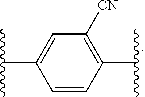 [SMILES: *c1ccc(*)c(C#N)c1]

In some embodiments, -Cy^{L3}- is

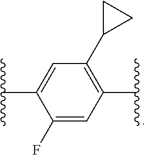 [SMILES: *c1cc(C2CC2)c(*)cc1F]

In some embodiments, -Cy^{L3}- is

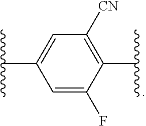 [SMILES: *c1cc(F)c(*)c(C#N)c1]

In some embodiments, -Cy^{L3}- is

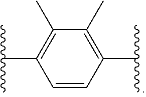 [SMILES: *c1ccc(*)c(C)c1C]

In some embodiments, -Cy^{L3}- is

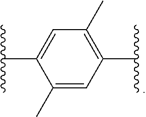 [SMILES: *c1cc(C)c(*)cc1C ; "."]

In some embodiments, -Cy^{L3}- is
In some embodiments, -Cy^{L3}- is

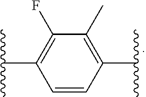 [SMILES: *c1ccc(*)c(F)c1C]

In some embodiments, -Cy^{L3}- is

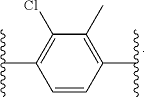 [SMILES: *c1ccc(*)c(Cl)c1C]

In some embodiments, -Cy^{L3}- is

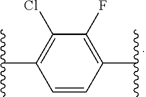 [SMILES: *c1ccc(*)c(Cl)c1F]

In some embodiments, -Cy^{L3}- is

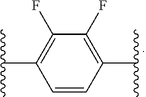 [SMILES: *c1ccc(*)c(F)c1F]

In some embodiments, -Cy^{L3}- is

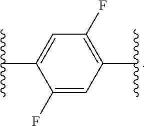 [SMILES: *c1cc(F)c(*)cc1F]

In some embodiments, -Cy^{L3}- is

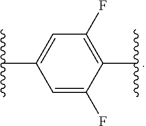 [SMILES: *c1cc(F)c(*)c(F)c1]

In some embodiments, -Cy^{L3}- is

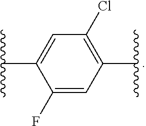 [SMILES: *c1cc(Cl)c(*)cc1F]

In some embodiments, -Cy^{L3}- is

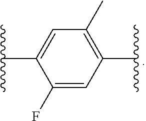 [SMILES: *c1cc(F)c(*)cc1C]

In some embodiments, -Cy^{L3}- is

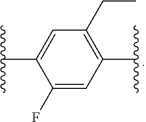 [SMILES: *c1cc(CC)c(*)cc1F]

In some embodiments, -Cy^{L3}- is

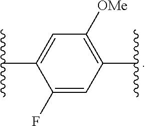 [SMILES: *c1cc(OC)c(*)cc1F]

In some embodiments, -Cy^{L3}- is

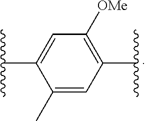 [SMILES: *c1cc(OC)c(*)cc1C]

In some embodiments, -Cy^{L3}- is

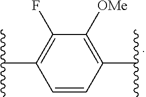 [SMILES: *c1ccc(*)c(OC)c1F]

In some embodiments, -Cy^{L3}- is

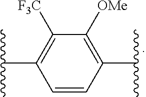 [SMILES: *c1ccc(*)c(C(F)(F)F)c1OC]

In some embodiments, -Cy^{L3}- is

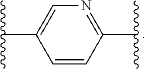 [SMILES: *c1ccc(*)nc1]

In some embodiments, -Cy^{L3}- is

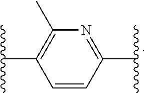 [SMILES: *c1ccc(*)c(C)n1]

In some embodiments, -Cy^{L3}- is

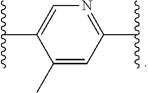 [SMILES: *c1cc(C)c(*)cn1]

In some embodiments, -Cy^{L3}- is

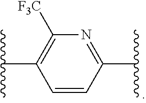 [SMILES: *c1ccc(*)c(C(F)(F)F)n1]

In some embodiments, -Cy^{L3}- is

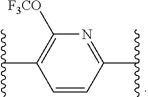 [SMILES: *c1ccc(*)c(OC(F)(F)F)n1]

In some embodiments, -Cy^{L3}- is

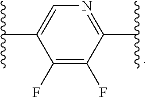 [SMILES: *c1cnc(*)c(F)c1F]

In some embodiments, -Cy^{L3}- is

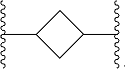 [SMILES: *C1CC(*)C1]

In some embodiments, -Cy^{L3}- is

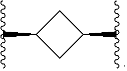 [SMILES: *[C@H]1C[C@@H](*)C1]

In some embodiments, -Cy^{L3}- is

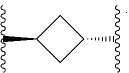 [SMILES: *[C@H]1C[C@H](*)C1]

In some embodiments, -Cy^{L3}- is

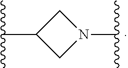 [SMILES: *C1CN(*)C1]

In some embodiments, -Cy^{L3}- is

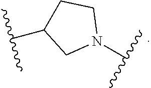 [SMILES: *C1CCN(*)C1]

In some embodiments, -Cy^{L3}- is

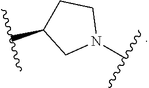 [SMILES: *[C@H]1CCN(*)C1]

In some embodiments, -Cy^{L3}- is

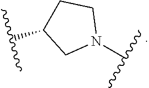 [SMILES: *[C@@H]1CCN(*)C1]

In some embodiments, -Cy^{L3}- is

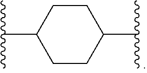 [SMILES: *C1CCC(*)CC1]

In some embodiments, -Cy^{L3}- is

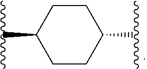 [SMILES: *[C@H]1CC[C@H](*)CC1]

In some embodiments, -Cy^{L3}- is

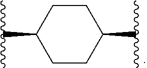 [SMILES: *[C@H]1CC[C@@H](*)CC1]

In some embodiments, -Cy^{L3}- is

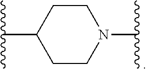 [SMILES: *C1CCN(*)CC1 ; "."]

In some embodiments, -Cy^{L3}- is

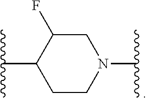 [SMILES: *C1CCN(*)CC1F ; "."]

In some embodiments, -Cy^{L3}- is

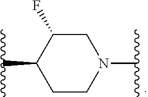 [SMILES: *[C@@H]1CCN(*)C[C@H]1F]

In some embodiments, -Cy^{L3}- is

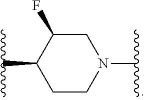 [SMILES: *[C@@H]1CCN(*)C[C@@H]1F]

In some embodiments, -Cy^{L3}- is

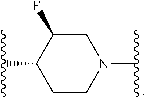 [SMILES: *[C@H]1CCN(*)C[C@@H]1F]

In some embodiments, -Cy^{L3}- is

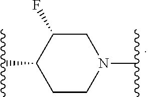 [SMILES: *[C@H]1CCN(*)C[C@H]1F]

In some embodiments, -Cy^{L3}- is

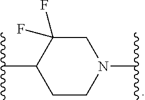 [SMILES: *C1CCN(*)CC1(F)F]

In some embodiments, -Cy^{L3}- is

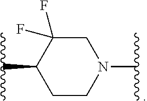 [SMILES: *[C@@H]1CCN(*)CC1(F)F ; "."]

In some embodiments, -Cy^{L3}- is

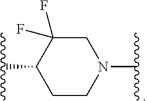 [SMILES: *[C@H]1CCN(*)CC1(F)F ; "."]

In some embodiments, -Cy^{L3}- is

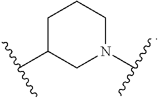 [SMILES: *C1CCCN(*)C1 ; "."]

In some embodiments, -Cy^{L3}- is

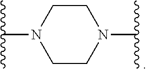 [SMILES: *N1CCN(*)CC1 ; "."]

In some embodiments, -Cy^{L3}- is

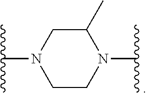 [SMILES: *N1CCN(*)C(C)C1 ; "."]

In some embodiments, -Cy^{L3}- is

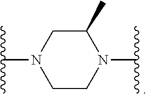 [SMILES: *N1CCN(*)[C@H](C)C1 ; "."]

In some embodiments, -Cy^{L3}- is

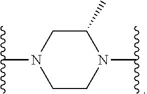 [SMILES: *N1CCN(*)[C@@H](C)C1 ; "."]

In some embodiments, -Cy^{L3}- is

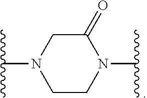 [SMILES: *N1CCN(*)C(=O)C1 ; "."]

In some embodiments, -Cy^{L3}- is

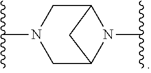 [SMILES: *N1CC2CC(C1)N2*]

In some embodiments, -Cy^{L3}- is

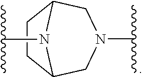 [SMILES: *N1CC2CCC(C1)N2*]

In some embodiments, -Cy^{L3}- is

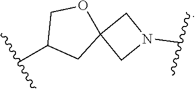 [SMILES: *C1COC2(C1)CN(*)C2]

In some embodiments, -Cy^{L3}- is

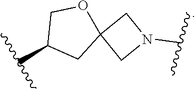 [SMILES: *[C@H]1COC2(C1)CN(*)C2]

In some embodiments, -Cy^{L3}- is

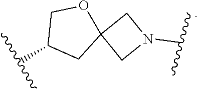 [SMILES: *[C@@H]1COC2(C1)CN(*)C2]

In some embodiments, -Cy^{L3}- is

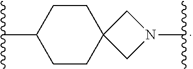 [SMILES: *C1CCC2(CC1)CN(*)C2]

In some embodiments, -Cy^{L3}- is

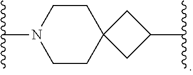 [SMILES: *C1CC2(CCN(*)CC2)C1]

In some embodiments, -Cy^{L3}- is

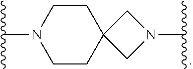 [SMILES: *N1CCC2(CC1)CN(*)C2]

In some embodiments, -Cy^{L3}- is

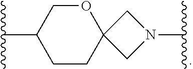 [SMILES: *C1CCC2(CN(*)C2)OC1]

In some embodiments, -Cy^{L3}- is

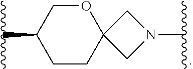 [SMILES: *[C@@H]1CCC2(CN(*)C2)OC1]

In some embodiments, -Cy^{L3}- is

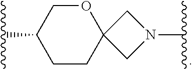 [SMILES: *[C@H]1CCC2(CN(*)C2)OC1]

In some embodiments, -Cy^{L3}- is

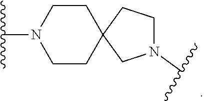 [SMILES: *N1CCC2(CC1)CCN(*)C2]

In some embodiments, -Cy^{L3}- is

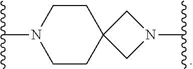 [SMILES: *N1CCC2(CC1)CN(*)C2]

In some embodiments, -Cy^{L3}- is

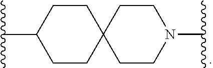 [SMILES: *C1CCC2(CC1)CCN(*)CC2]

In some embodiments, -Cy^{L3}- is

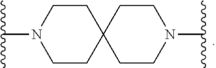 [SMILES: *N1CCC2(CC1)CCN(*)CC2]

In some embodiments, -Cy^{L3}- is

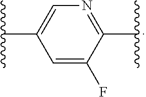 [SMILES: *c1cnc(*)c(F)c1]

In some embodiments, -Cy^{L4}- is an optionally substituted 8-10 membered bicyclic arylenyl. In some embodiments, -Cy^{L4}- is an optionally substituted 3-7 membered saturated or partially unsaturated carbocyclylenyl. In some embodiments, -Cy^{L4}- is an optionally substituted 6-11 membered saturated or partially unsaturated spiro carbocyclylenyl. In some embodiments, -Cy^{L4}- is an 8-10 membered bicyclic saturated or partially unsaturated carbocyclylenyl. In some embodiments, -Cy^{L4}- is an optionally substituted a 4-7 membered saturated or partially unsaturated heterocyclylenyl having 1-2 heteroatoms independently selected from nitrogen, oxygen, and sulfur. In some embodiments, -Cy^{L1}- is an optionally substituted 6-11 membered saturated or partially unsaturated spiro heterocyclylenyl having 1-3 heteroatoms independently selected from nitrogen, oxygen, and sulfur. In some embodiments, -Cy^{L4}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L4}- is an optionally substituted 5-6 membered heteroarylenyl having 1-4 heteroatoms independently selected from nitrogen, oxygen. In some embodiments, -Cy^{L4}- is an optionally substituted 8-10 membered bicyclic heteroarylenyl having 1-5 heteroatoms independently selected from nitrogen, oxygen, and sulfur.

In some embodiments, each -Cy^{L4}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an 8-10 membered bicyclic arylenyl. In some embodiments, -Cy^{L4}- is a 3-7 membered saturated or partially unsaturated carbocyclylenyl. In some embodiments, -Cy^{L4}- is a 6-11 membered saturated or partially unsaturated spiro carbocyclylenyl. In some embodiments, -Cy^{L4}- is an 8-10 membered bicyclic saturated or partially unsaturated carbocyclylenyl. In some embodiments, -Cy^{L4}- is a 4-7 membered saturated or partially unsaturated heterocyclylenyl having 1-2 heteroatoms independently selected from nitrogen, oxygen, and sulfur. In some embodiments, -Cy^{L4}- is a 6-11 membered saturated or partially unsaturated spiro heterocyclylenyl having 1-3 heteroatoms independently selected from nitrogen, oxygen, and sulfur. In some embodiments, -Cy^{L4}- is an 8-10 membered bicyclic saturated or partially unsaturated heterocyclylenyl having 1-2 heteroatoms independently selected from nitrogen, oxygen, and sulfur. In some embodiments, -Cy^{L4}- is a 5-6 membered heteroarylenyl having 1-4 heteroatoms independently selected from nitrogen, oxygen. In some embodiments, -Cy^{L1}- is an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L4}- is an optionally substituted phenylenyl. In some embodiments, -Cy^{L4}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is phenylenyl, optionally substituted with halogen (e.g., fluoro or chloro) or C₁₋₆ aliphatic.

In some embodiments, -Cy^{L4}- is an optionally substituted 5-6 membered saturated or partially unsaturated heterocyclylenyl having 1-2 heteroatoms independently selected from nitrogen, oxygen, and sulfur. In some embodiments, -Cy^{L4}- is an optionally substituted 5 membered saturated or partially unsaturated heterocyclylenyl having 1-2 heteroatoms independently selected from nitrogen, oxygen, and sulfur. In some embodiments, -Cy^{L4}- is an optionally substituted pyrrolidinylenyl. In some embodiments, -Cy^{L4}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyrrolidinylenyl, optionally substituted with halogen (e.g., fluoro or chloro) or C₁₋₆ aliphatic. In some embodiments, -Cy^{L1}- is an optionally substituted 6 membered saturated or partially unsaturated heterocyclylenyl having 1-2 heteroatoms independently selected from nitrogen, oxygen, and sulfur. In some embodiments, -Cy^{L4}- is an optionally substituted piperadinylenyl. In some embodiments, -Cy^{L4}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is piperadinylenyl, optionally substituted with halogen (e.g., fluoro or chloro) or C₁₋₆ aliphatic. In some embodiments, -Cy^{L4}- is an optionally substituted piperazinylenyl. In some embodiments, -Cy^{L4}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is piperazinylenyl, optionally substituted with halogen (e.g., fluoro or chloro) or C₁₋₆ aliphatic.

In some embodiments, -Cy^{L1}- is an optionally substituted 5 membered heteroarylenyl having 1-4 heteroatoms independently selected from nitrogen, oxygen. In some embodiments, -Cy^{L4}- is an optionally substituted pyrrolylenyl, pyrazolylenyl, imidazolylenyl, or triazolylenyl. In some embodiments, -Cy^{L4}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L4}- is an optionally substituted pyrazolylenyl. In some embodiments, -Cy^{L4}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₆ aliphatic. In some embodiments, -Cy^{L4}- is pyrazolylenyl, optionally substituted with halogen (e.g., fluoro or chloro) or C₁₋₆ aliphatic. In some embodiments, -Cy^{L4}- is an optionally substituted imidazolylenyl. In some embodiments, -Cy^{L4}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L1}- is imidazolylenyl, optionally substituted with halogen (e.g., fluoro or chloro) or C₁₋₆ aliphatic.

In some embodiments, -Cy^{L4}- is an optionally substituted 6 membered heteroarylenyl having 1-4 heteroatoms independently selected from nitrogen, oxygen. In some embodiments, -Cy^{L1}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L1}- is an optionally substituted pyridinylenyl. In some embodiments, -Cy^{L4}- is pyridinylenyl, optionally substituted with halogen (e.g., fluoro or chloro), —CN, C₁₋₆ aliphatic optionally further substituted with halogen (e.g., fluoro or chloro), —S(O)₂R^{o} or —OR^{o}, wherein R^{o} is hydrogen or C₁₋₆ aliphatic. In some embodiments, -Cy^{L4}- is pyridinylenyl, optionally substituted with halogen (e.g., fluoro or chloro) or C₁₋₆ aliphatic. In some embodiments, -Cy^{L4}- is an optionally substituted pyridazinylenyl. In some embodiments, -Cy^{L4}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridazinylenyl, optionally substituted with halogen (e.g., fluoro or chloro) or C₁₋₆ aliphatic. In some embodiments, -Cy^{L4}- is an optionally substituted pyridinonylenyl, pyridazinonylenyl, pyrimidinonylenyl, pyrazinonylenyl, or triazinonylenyl. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L4}- is an optionally substituted pyridinonylenyl. In some embodiments, -Cy^{L4}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridinonylenyl, optionally substituted with halogen (e.g., fluoro or chloro) or C₁₋₆ aliphatic.

In some embodiments, -Cy^{L2}- is an optionally substituted cyclopropyl. In some embodiments, -Cy^{L4}- is an optionally substituted 5 membered saturated or partially unsaturated carbocyclylenyl. In some embodiments, -Cy^{L4}- is an optionally substituted cyclopentanylenyl or cyclopentenylenyl. In some embodiments, -Cy^{L4}- is an optionally substituted 6 membered saturated or partially unsaturated carbocyclylenyl. In some embodiments, -Cy^{L4}- is an optionally substituted cyclohexanylenyl or cyclohexenylenyl. In some embodiments, -Cy^{L4}- is an optionally substituted naphthalenylenyl.

In some embodiments, -Cy^{L4}- is an optionally substituted 9 membered bicyclic heteroarylenyl having 1-5 heteroatoms independently selected from nitrogen, oxygen, and sulfur. In some embodiments, -Cy^{L4}- is an optionally substituted indolenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L4} is an optionally substituted indolylenyl. In some embodiments, -Cy^{L4}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L4}- is an optionally substituted 8-11 membered saturated or partially unsaturated spiro heterocyclylenyl having 1-3 heteroatoms independently selected from nitrogen, oxygen, and sulfur. In some embodiments, -Cy^{L4}- is an optionally substituted 9-11 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 nitrogen heteroatoms. In some embodiments, -Cy^{L4}- is an optionally substituted 4,6-spiro heterocyclylenyl having 1-3 heteroatoms independently selected from nitrogen, oxygen, and sulfur. In some embodiments, -Cy- is an optionally substituted

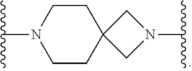 [SMILES: *N1CCC2(CC1)CN(*)C2]

In some embodiments, -Cy^{L4}- is an optionally substituted 10 membered saturated or partially unsaturated spiro heterocyclylenyl having 1-3 heteroatoms independently selected from nitrogen, oxygen, and sulfur. In some embodiments, -Cy^{L4}- is an optionally substituted 10 membered saturated or partially unsaturated spiro heterocyclylenyl having 1-3 nitrogen heteroatoms. In some embodiments, -Cy^{L4}- is an optionally substituted 5,6-spiro heterocyclylenyl having 1-3 heteroatoms independently selected from nitrogen, oxygen, and sulfur.

In some embodiments, -Cy^{L4}- is an optionally substituted 11 membered saturated or partially unsaturated spiro heterocyclylenyl having 1-3 heteroatoms independently selected from nitrogen, oxygen, and sulfur. In some embodiments, -Cy^{L4}- is an optionally substituted 11 membered saturated or partially unsaturated spiro heterocyclylenyl having 1-3 nitrogen heteroatoms. In some embodiments, -Cy^{L1}- is an optionally substituted 6,6-spiro heterocyclylenyl having 1-3 heteroatoms independently selected from nitrogen, oxygen, and sulfur. In some embodiments, -Cy^{L4}- is an optionally substituted

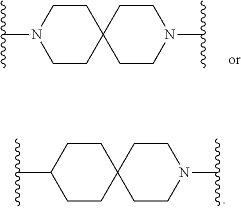 [SMILES: *N1CCC2(CC1)CCN(*)CC2 ; *C1CCC2(CC1)CCN(*)CC2]

In some embodiments, -Cy^{L4}- is optionally substituted

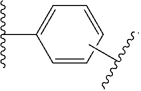

In some embodiments, -Cy^{L4}- is

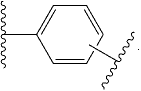

In some embodiments, -Cy^{L4}- is

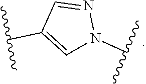 [SMILES: *c1cnn(*)c1]

In some embodiments, -Cy^{L4}- is

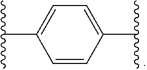 [SMILES: *c1ccc(*)cc1]

In some embodiments, -Cy^{L4}- is

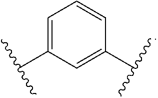 [SMILES: *c1cccc(*)c1]

In some embodiments, -Cy^{L4}- is

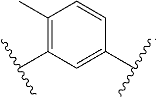 [SMILES: *c1ccc(C)c(*)c1]

In some embodiments, -Cy^{L4}- is

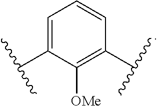 [SMILES: *c1cccc(*)c1OC]

In some embodiments, -Cy^{L4}- is

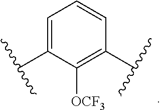 [SMILES: *c1cccc(*)c1OC(F)(F)F]

In some embodiments, -Cy^{L4}- is

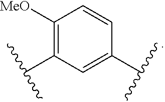 [SMILES: *c1ccc(OC)c(*)c1]

In some embodiments, -Cy^{L4}- is

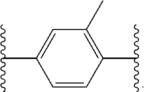 [SMILES: *c1ccc(*)c(C)c1]

In some embodiments, -Cy^{L4}- is

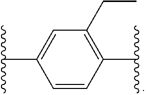 [SMILES: *c1ccc(*)c(CC)c1]

In some embodiments, -Cy^{L4}- is

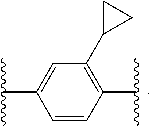 [SMILES: *c1ccc(*)c(C2CC2)c1]

In some embodiments, -Cy^{L4}- is

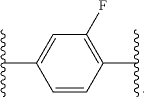 [SMILES: *c1ccc(*)c(F)c1]

In some embodiments, -Cy^{L4}- is

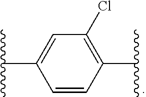 [SMILES: *c1ccc(*)c(Cl)c1]

In some embodiments, -Cy^{L4}- is

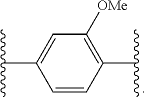 [SMILES: *c1ccc(*)c(OC)c1]

In some embodiments, -Cy^{L4}- is

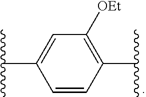 [SMILES: *c1ccc(*)c(OCC)c1]

In some embodiments, -Cy^{L4}- is

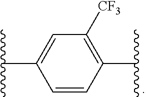 [SMILES: *c1ccc(*)c(C(F)(F)F)c1]

In some embodiments, -Cy^{L4}- is

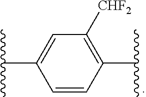 [SMILES: *c1ccc(*)c(C(F)F)c1]

In some embodiments, -Cy^{L4}- is

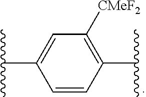

In some embodiments, -Cy^{L4}- is

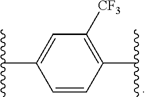 [SMILES: *c1ccc(*)c(C(F)(F)F)c1]

In some embodiments, -Cy^{L4}- is

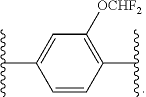 [SMILES: *c1ccc(*)c(OC(F)F)c1]

In some embodiments, -Cy^{L4}- is

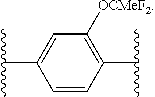

In some embodiments, -Cy^{L4}- is

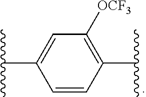 [SMILES: *c1ccc(*)c(OC(F)(F)F)c1]

In some embodiments, -Cy^{L4}- is

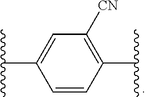 [SMILES: *c1ccc(*)c(C#N)c1]

In some embodiments, -Cy^{L4}- is

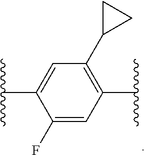 [SMILES: *c1cc(C2CC2)c(*)cc1F]

In some embodiments, -Cy^{L4}- is

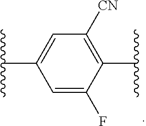 [SMILES: *c1cc(F)c(*)c(C#N)c1]

In some embodiments, -Cy^{L4}- is

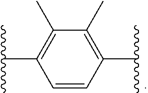 [SMILES: *c1ccc(*)c(C)c1C]

In some embodiments, -Cy^{L4}- is

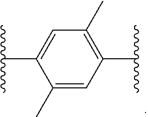 [SMILES: *c1cc(C)c(*)cc1C]

In some embodiments, -Cy^{L4}- is

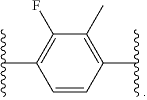 [SMILES: *c1ccc(*)c(F)c1C]

In some embodiments, -Cy^{L4}- is

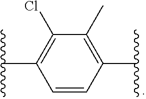 [SMILES: *c1ccc(*)c(Cl)c1C]

In some embodiments, -Cy^{L4}- is

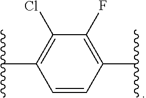 [SMILES: *c1ccc(*)c(Cl)c1F ; "."]

In some embodiments, -Cy^{L4}- is

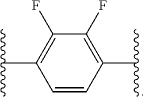 [SMILES: *c1ccc(*)c(F)c1F]

In some embodiments, -Cy^{L4}- is

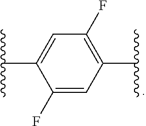 [SMILES: *c1cc(F)c(*)cc1F]

In some embodiments, -Cy^{L4}- is

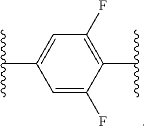 [SMILES: *c1cc(F)c(*)c(F)c1]

In some embodiments, -Cy^{L4}- is

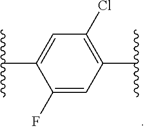 [SMILES: *c1cc(Cl)c(*)cc1F]

In some embodiments, -Cy^{L4}- is

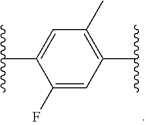 [SMILES: *c1cc(F)c(*)cc1C]

In some embodiments, -Cy^{L4}- is

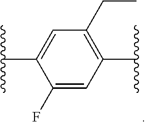 [SMILES: *c1cc(CC)c(*)cc1F]

In some embodiments, -Cy^{L4}- is

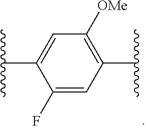 [SMILES: *c1cc(OC)c(*)cc1F]

In some embodiments, -Cy^{L4}- is

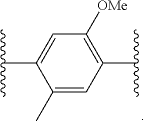 [SMILES: *c1cc(OC)c(*)cc1C]

In some embodiments, -Cy^{L4}- is

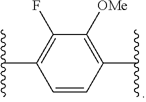 [SMILES: *c1ccc(*)c(OC)c1F]

In some embodiments, -Cy^{L4}- is

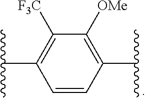 [SMILES: *c1ccc(*)c(C(F)(F)F)c1OC]

In some embodiments, -Cy^{L4}- is

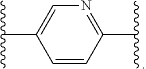 [SMILES: *c1ccc(*)nc1 ; "."]

In some embodiments, -Cy^{L4}- is

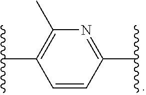 [SMILES: *c1ccc(*)c(C)n1]

In some embodiments, -Cy^{L4}- is

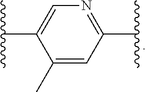 [SMILES: *c1cc(C)c(*)cn1]

In some embodiments, -Cy^{L4}- is

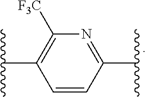 [SMILES: *c1ccc(*)c(C(F)(F)F)n1]

In some embodiments, -Cy^{L4}- is

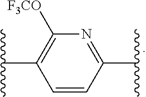 [SMILES: *c1ccc(*)c(OC(F)(F)F)n1]

In some embodiments, -Cy^{L4}- is

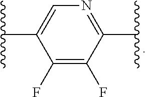 [SMILES: *c1cnc(*)c(F)c1F]

In some embodiments, -Cy^{L4}- is

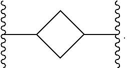 [SMILES: *C1CC(*)C1]

In some embodiments, -Cy^{L4}- is

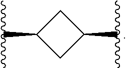 [SMILES: *[C@H]1C[C@@H](*)C1]

In some embodiments, -Cy^{L4}- is

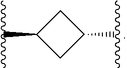 [SMILES: *[C@H]1C[C@H](*)C1]

In some embodiments, -Cy^{L4}- is

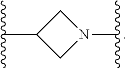 [SMILES: *C1CN(*)C1]

In some embodiments, -Cy^{L4}- is

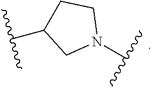 [SMILES: *C1CCN(*)C1]

In some embodiments, -Cy^{L4}- is

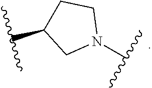 [SMILES: *[C@H]1CCN(*)C1]

In some embodiments, -Cy^{L4}- is

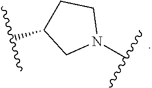 [SMILES: *[C@@H]1CCN(*)C1]

In some embodiments, -Cy^{L4}- is

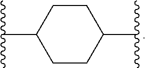 [SMILES: *C1CCC(*)CC1]

In some embodiments, -Cy^{L4}- is

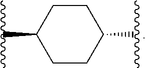 [SMILES: *[C@H]1CC[C@H](*)CC1]

In some embodiments, -Cy^{L4}- is

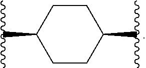 [SMILES: *[C@H]1CC[C@@H](*)CC1]

In some embodiments, -Cy^{L4}- is

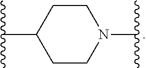 [SMILES: *C1CCN(*)CC1]

In some embodiments, -Cy^{L4}- is

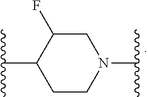 [SMILES: *C1CCN(*)CC1F]

In some embodiments, -Cy^{L4}- is

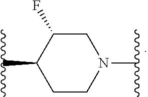 [SMILES: *[C@@H]1CCN(*)C[C@H]1F]

In some embodiments, -Cy^{L4}- is

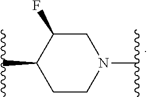 [SMILES: *[C@@H]1CCN(*)C[C@@H]1F]

In some embodiments, -Cy^{L4}- is

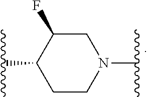 [SMILES: *[C@H]1CCN(*)C[C@@H]1F]

In some embodiments, -Cy^{L4}- is

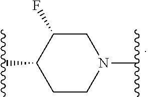 [SMILES: *[C@H]1CCN(*)C[C@H]1F]

In some embodiments, -Cy^{L4}- is

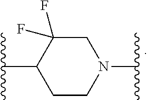 [SMILES: *C1CCN(*)CC1(F)F]

In some embodiments, -Cy^{L4}- is

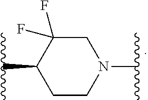 [SMILES: *[C@@H]1CCN(*)CC1(F)F]

In some embodiments, -Cy^{L4}- is

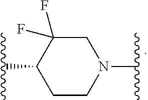 [SMILES: *[C@H]1CCN(*)CC1(F)F]

In some embodiments, -Cy^{L4}- is

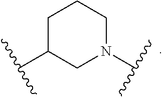 [SMILES: *C1CCCN(*)C1]

In some embodiments, -Cy^{L4}- is

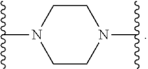 [SMILES: *N1CCN(*)CC1]

In some embodiments, -Cy^{L4}- is

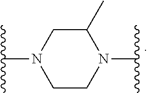 [SMILES: *N1CCN(*)C(C)C1]

In some embodiments, -Cy^{L4}- is

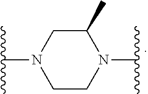 [SMILES: *N1CCN(*)[C@H](C)C1]

In some embodiments, -Cy^{L4}- is

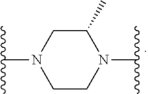 [SMILES: *N1CCN(*)[C@@H](C)C1]

In some embodiments, -Cy^{L4}- is

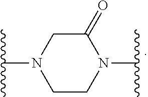 [SMILES: *N1CCN(*)C(=O)C1]

In some embodiments, -Cy^{L4}- is

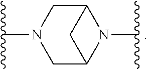 [SMILES: *N1CC2CC(C1)N2*]

In some embodiments, -Cy^{L4}- is

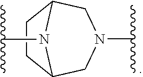 [SMILES: *N1CC2CCC(C1)N2*]

In some embodiments, -Cy^{L4}- is

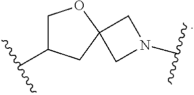 [SMILES: *C1COC2(C1)CN(*)C2]

In some embodiments, -Cy^{L4}- is

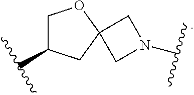 [SMILES: *[C@H]1COC2(C1)CN(*)C2]

In some embodiments, -Cy^{L4}- is

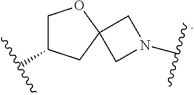 [SMILES: *[C@@H]1COC2(C1)CN(*)C2]

In some embodiments, -Cy^{L4}- is

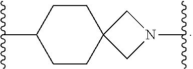 [SMILES: *C1CCC2(CC1)CN(*)C2]

In some embodiments, -Cy^{L4}- is

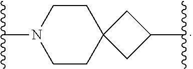 [SMILES: *C1CC2(CCN(*)CC2)C1]

In some embodiments, -Cy^{L4}- is

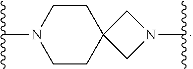 [SMILES: *N1CCC2(CC1)CN(*)C2]

In some embodiments, -Cy^{L4}- is

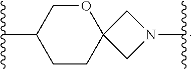 [SMILES: *C1CCC2(CN(*)C2)OC1]

In some embodiments, -Cy^{L4}- is

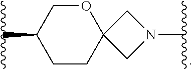 [SMILES: *[C@@H]1CCC2(CN(*)C2)OC1]

In some embodiments, -Cy^{L4}- is

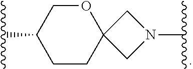 [SMILES: *[C@H]1CCC2(CN(*)C2)OC1]

In some embodiments, -Cy^{L4}- is

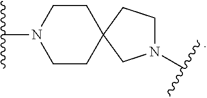 [SMILES: *N1CCC2(CC1)CCN(*)C2]

In some embodiments, -Cy^{L4}- is

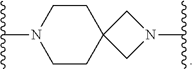 [SMILES: *N1CCC2(CC1)CN(*)C2]

In some embodiments, -Cy^{L4}- is

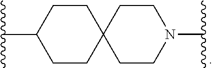 [SMILES: *C1CCC2(CC1)CCN(*)CC2]

In some embodiments, -Cy^{L4}- is

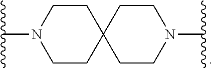 [SMILES: *N1CCC2(CC1)CCN(*)CC2]

In some embodiments, -Cy^{L4}- is

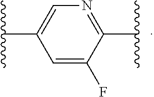 [SMILES: *c1cnc(*)c(F)c1]

In some embodiments, L is:

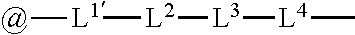

wherein
@ represents the point of attachment to Ring W;
L¹ is -Cy^{L1}-;
L² is -Cy^{L2}-;
L³ is -Cy^{L3}; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

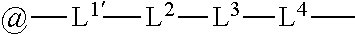

wherein
@ represents the point of attachment to Ring W;
L¹ is -Cy^{L1}-:
L² is -Cy^{L2}-;
L³ is -Cy^{L1}-; and
L⁴ is —C(O)—.

In some embodiments, L is:

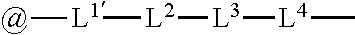

wherein
@ represents the point of attachment to Ring W;
L¹ is -Cy^{L1}-;
L² is -Cy^{L1}-:
L³ is -Cy^{L3}-;
L⁴ is a covalent bond or an optionally substituted bivalent, saturated or partially unsaturated, straight or branched C₁₋₆ hydrocarbon chain.

In some embodiments, L is:

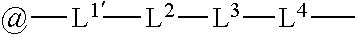

wherein
@ represents the point of attachment to Ring W;
L¹ is -Cy^{L1}-:
L² is -Cy^{L2}-;
L³ is -Cy^{L3}-;
L⁴ is an optionally substituted bivalent, saturated or partially unsaturated, straight or branched C₁₋₆ hydrocarbon chain.

In some embodiments, L is:

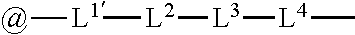

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-:
L³ is -Cy^{L3}; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

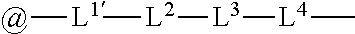

wherein
@ represents the point of attachment to Ring W;
L¹ is an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-;
L³ is -Cy^{L3}-; and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

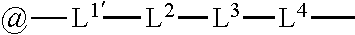

wherein
@ represents the point of attachment to Ring W;
L¹ is an optionally substituted bivalent, saturated or partially unsaturated, straight or branched C₁₋₆ hydrocarbon chain;
L² is -Cy^{L2}-;
L³ is -Cy^{L3}-; and
L⁴ is an optionally substituted bivalent, saturated or partially unsaturated, straight or branched C₁₋₆ hydrocarbon chain.

In some embodiments, L is:

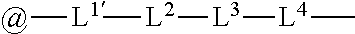

wherein
@ represents the point of attachment to Ring W;
L¹ is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy-L³; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

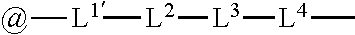

wherein
@ represents the point of attachment to Ring W;
L¹ is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy^{L3}-; and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

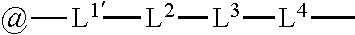

wherein
@ represents the point of attachment to Ring W;
L¹ is -Cy^{L1}-;
L² is an optionally substituted bivalent, saturated or partially unsaturated, straight or branched C₁₋₆ hydrocarbon chain;
L³ is -Cy^{L3}; and
L⁴ is an optionally substituted bivalent, saturated or partially unsaturated, straight or branched C₁₋₆ hydrocarbon chain.

In some embodiments, L is:

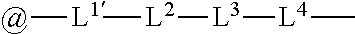

wherein
@ represents the point of attachment to Ring W;
L¹ is -Cy^{L1}-;
L² is -Cy^{L2}-;
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

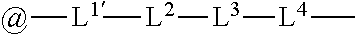

wherein
@ represents the point of attachment to Ring W;
L¹ is -Cy^{L1}-;
L² is -Cy^{L2}-;
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

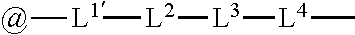

wherein
@ represents the point of attachment to Ring W;
L¹ is -Cy^{L1}-;
L² is -Cy^{L2}-;
L³ is an optionally substituted bivalent, saturated or partially unsaturated, straight or branched C₁₋₆ hydrocarbon chain; and
L⁴ is -Cy^{L4}-.

In some embodiments, L is:

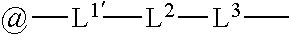

wherein
@ represents the point of attachment to Ring W;
L¹ is -Cy^{L1}-;
L² is -Cy^{L2}; and
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

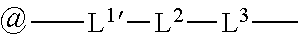

wherein
@ represents the point of attachment to Ring W;
L¹ is -Cy^{L1}-;
L² is -Cy^{L2}-; and
L³ is a covalent bond or an optionally substituted bivalent, saturated or partially unsaturated, straight or branched C₁₋₆ hydrocarbon chain.

In some embodiments, L is:

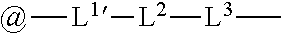

wherein
@ represents the point of attachment to Ring W;
L¹ is -Cy^{L1}-;
L² is -Cy^{L2}-; and
L³ is an optionally substituted bivalent, saturated or partially unsaturated, straight or branched C₁₋₆ hydrocarbon chain.

In some embodiments, L is:

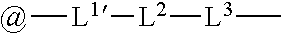

wherein
@ represents the point of attachment to Ring W;
L^{1′} is -Cy^{L1}-;
L² is -Cy^{L2}; and
L³ is -Cy^{L3}-.

In some embodiments, L is:

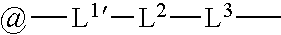

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}; and
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

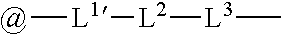

wherein
@ represents the point of attachment to Ring W;
L^{1′} is an optionally substituted bivalent, saturated or partially unsaturated, straight or branched C₁₋₆ hydrocarbon chain, wherein 1-2 methylene units of L^{1′} are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}; and
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

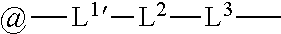

wherein
@ represents the point of attachment to Ring W;
L¹ is an optionally substituted bivalent, saturated or partially unsaturated, straight or branched C₁₋₆ hydrocarbon chain;
L² is -Cy^{L2}-; and
L³ is an optionally substituted bivalent, saturated or partially unsaturated, straight or branched C₁₋₆ hydrocarbon chain.

In some embodiments, L is:

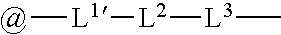

wherein
@ represents the point of attachment to Ring W;
L¹ is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy^{L3}-;

In some embodiments, L is:

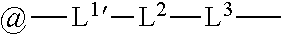

wherein
@ represents the point of attachment to Ring W;
L¹ is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy^{L1}-;

In some embodiments, L is:

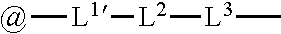

wherein
@ represents the point of attachment to Ring W;
L¹ is -Cy^{L1}-;
L² is an optionally substituted bivalent, saturated or partially unsaturated, straight or branched C₁₋₆ hydrocarbon chain; and
L³ is -Cy^{L3}-.

In some embodiments, L is:

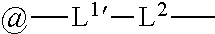

wherein
@ represents the point of attachment to Ring W;
L¹ is -Cy^{L1}-; and
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

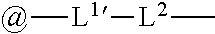

wherein
@ represents the point of attachment to Ring W;
L¹ is -Cy^{L1}-; and
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

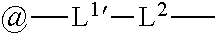

wherein
@ represents the point of attachment to Ring W;
L¹ is -Cy^{L1}-; and
L² is an optionally substituted bivalent, saturated or partially unsaturated, straight or branched C₁₋₆ hydrocarbon chain.

In some embodiments, L is:

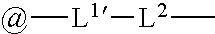

wherein
@ represents the point of attachment to Ring W;
L¹ is -Cy^{L1}-; and
L² is -Cy^{L2}-.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-3 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, each -Cy- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently bivalent phenylenyl. In some embodiments, each -Cy- is independently an 8-10 membered bicyclic arylenyl. In some embodiments, each -Cy- is independently a 4-7 membered saturated or partially unsaturated carbocyclylenyl. In some embodiments, each -Cy- is independently a 6-11 membered saturated or partially unsaturated spiro carbocyclylenyl. In some embodiments, each -Cy- is independently an 8-10 membered bicyclic saturated or partially unsaturated carbocyclylenyl. In some embodiments, each -Cy- is independently a 4-7 membered saturated or partially unsaturated heterocyclylenyl having 1-2 heteroatoms independently selected from nitrogen, oxygen, and sulfur. In some embodiments, each -Cy- is independently a 6-11 membered saturated or partially unsaturated spiro heterocyclylenyl having 1-3 heteroatoms independently selected from nitrogen, oxygen, and sulfur. In some embodiments, each -Cy- is independently an 8-10 membered bicyclic saturated or partially unsaturated heterocyclylenyl having 1-2 heteroatoms independently selected from nitrogen, oxygen, and sulfur. In some embodiments, each -Cy- is independently a 5-6 membered heteroarylenyl having 1-4 heteroatoms independently selected from nitrogen, oxygen, and sulfur. In some embodiments, each -Cy- is independently an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy- is substituted with halogen. In some embodiments, -Cy- is substituted with —CN. In some embodiments, -Cy- is substituted with C₁₋₆ alkyl (e.g., methyl, ethyl, isopropyl). In some embodiments, -Cy- is substituted with C₃₋₆ cycloalkyl. In some embodiments, -Cy- is substituted with C₁₋₆ haloalkyl (e.g., —CHF₂, —CF₃). In some embodiments, -Cy- is substituted with —OC₁₋₁₀ alkyl (e.g., —OMe, —OEt). In some embodiments, -Cy- is substituted with —OC₁₋₆ haloalkyl (e.g., —OCHF₂, —OCF₃).

In some embodiments, -Cy- is substituted with methyl. In some embodiments, -Cy- is substituted with ethyl. In some embodiments, -Cy- is substituted with cyclopropyl. In some embodiments, -Cy- is substituted with —CHF₂. In some embodiments, -Cy- is substituted with —CMeF₂. In some embodiments, -Cy- is substituted with —CF₃. In some embodiments, -Cy- is substituted with —OCHF₂. In some embodiments, -Cy- is substituted with —OCMeF₂. In some embodiments, -Cy- is substituted with —OCF₃. In some embodiments, -Cy- is substituted with —C(Me)OH. In some embodiments, -Cy- is substituted with oxo. In some embodiments, -Cy- is substituted with fluoro. In some embodiments, -Cy- is substituted with geminal difluoro. In some embodiments, -Cy- is substituted with —OH. In some embodiments, -Cy- is substituted with —OMe. In some embodiments, -Cy- is substituted with —OEt. In some embodiments, -Cy- is substituted with —NR₂.

In some embodiments, -Cy- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is phenylenyl, optionally substituted with halogen (e.g., fluoro or chloro) or C₁₋₆ aliphatic.

In some embodiments, -Cy- is an optionally substituted 5 membered saturated or partially unsaturated heterocyclylenyl having 1-2 heteroatoms independently selected from nitrogen, oxygen, and sulfur. In some embodiments, -Cy- is an optionally substituted pyrrolidinylenyl. In some embodiments, -Cy- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrrolidinylenyl, optionally substituted with halogen (e.g., fluoro or chloro) or C₁₋₆ aliphatic. In some embodiments, -Cy^{L4}- is an optionally substituted 6 membered saturated or partially unsaturated heterocyclylenyl having 1-2 heteroatoms independently selected from nitrogen, oxygen, and sulfur. In some embodiments, -Cy- is an optionally substituted piperadinylenyl. In some embodiments, -Cy- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is piperadinylenyl, optionally substituted with halogen (e.g., fluoro or chloro) or C₁₋₆ aliphatic. In some embodiments, -Cy- is an optionally substituted piperazinylenyl. In some embodiments, -Cy- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is piperazinylenyl, optionally substituted with halogen (e.g., fluoro or chloro) or C₁₋₆ aliphatic.

In some embodiments, -Cy- is an optionally substituted 5 membered heteroarylenyl having 1-4 heteroatoms independently selected from nitrogen, oxygen. In some embodiments, -Cy- is an optionally substituted pyrrolylenyl, pyrazolylenyl, imidazolylenyl, or triazolylenyl. In some embodiments, -Cy- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy- is an optionally substituted pyrazolylenyl. In some embodiments, -Cy- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrazolylenyl, optionally substituted with halogen (e.g., fluoro or chloro) or C₁₋₆ aliphatic. In some embodiments, -Cy- is an optionally substituted imidazolylenyl. In some embodiments, -Cy- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy- is imidazolylenyl, optionally substituted with halogen (e.g., fluoro or chloro) or C₁₋₆ aliphatic.

In some embodiments, -Cy- is an optionally substituted 6 membered heteroarylenyl having 1-4 heteroatoms independently selected from nitrogen, oxygen. In some embodiments, -Cy- is an optionally substituted pyridinylenyl, pyridazinylenyl, pyrimidinylenyl, pyrazinylenyl, or triazinylenyl. In some embodiments, -Cy- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy- is an optionally substituted pyridinylenyl. In some embodiments, -Cy- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinylenyl, optionally substituted with halogen (e.g., fluoro or chloro) or C₁₋₆ aliphatic. In some embodiments, -Cy- is an optionally substituted pyridazinylenyl. In some embodiments, -Cy- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridazinylenyl, optionally substituted with halogen (e.g., fluoro or chloro) or C₁₋₆ aliphatic. In some embodiments, -Cy- is an optionally substituted pyridinonylenyl, pyridazinonylenyl, pyrimidinonylenyl, pyrazinonylenyl, or triazinonylenyl. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy- is an optionally substituted pyridinonylenyl. In some embodiments, -Cy- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinonylenyl, optionally substituted with halogen (e.g., fluoro or chloro) or C₁₋₆ aliphatic.

In some embodiments, -Cy- is an optionally substituted cyclopropyl. In some embodiments, -Cy^{L4}- is an optionally substituted 5 membered saturated or partially unsaturated carbocyclylenyl. In some embodiments, -Cy- is an optionally substituted cyclopentanylenyl or cyclopentenylenyl. In some embodiments, -Cy- is an optionally substituted 6 membered saturated or partially unsaturated carbocyclylenyl. In some embodiments, -Cy- is an optionally substituted cyclohexanylenyl or cyclohexenylenyl. In some embodiments, -Cy- is an optionally substituted naphthalenylenyl.

In some embodiments, -Cy- is an optionally substituted 9 membered bicyclic heteroarylenyl having 1-5 heteroatoms independently selected from nitrogen, oxygen, and sulfur. In some embodiments, -Cy- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy- is an optionally substituted indolylenyl. In some embodiments, -Cy- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy- is an optionally substituted 9-11 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 nitrogen heteroatoms. In some embodiments, -Cy- is an optionally substituted 4,6-spiro heterocyclylenyl having 1-3 heteroatoms independently selected from nitrogen, oxygen, and sulfur. In some embodiments, -Cy- is an optionally substituted

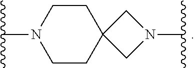 [SMILES: *N1CCC2(CC1)CN(*)C2]

In some embodiments, -Cy- is an optionally substituted 10 membered saturated or partially unsaturated spiro heterocyclylenyl having 1-3 heteroatoms independently selected from nitrogen, oxygen, and sulfur. In some embodiments, -Cy- is an optionally substituted 10 membered saturated or partially unsaturated spiro heterocyclylenyl having 1-3 nitrogen heteroatoms. In some embodiments, -Cy- is an optionally substituted 5,6-spiro heterocyclylenyl having 1-3 heteroatoms independently selected from nitrogen, oxygen, and sulfur.

In some embodiments, -Cy- is an optionally substituted 11 membered saturated or partially unsaturated spiro heterocyclylenyl having 1-3 heteroatoms independently selected from nitrogen, oxygen, and sulfur. In some embodiments, -Cy- is an optionally substituted 11 membered saturated or partially unsaturated spiro heterocyclylenyl having 1-3 nitrogen heteroatoms. In some embodiments, -Cy- is an optionally substituted 6,6-spiro heterocyclylenyl having 1-3 heteroatoms independently selected from nitrogen, oxygen, and sulfur.

In some embodiments, -Cy- is

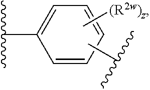

Wherein R^{2w} and z are defined above and as described herein.

In some embodiments, -Cy- is

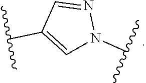 [SMILES: *c1cnn(*)c1]

In some embodiments, -Cy- is

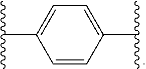 [SMILES: *c1ccc(*)cc1]

In some embodiments, -Cy- is

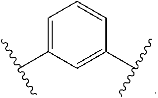 [SMILES: *c1cccc(*)c1]

In some embodiments, -Cy- is

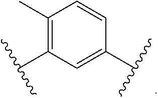 [SMILES: *c1ccc(C)c(*)c1]

In some embodiments, -Cy- is

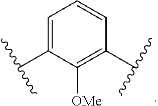 [SMILES: *c1cccc(*)c1OC]

In some embodiments, -Cy- is

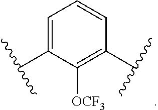 [SMILES: *c1cccc(*)c1OC(F)(F)F]

In some embodiments, -Cy- is

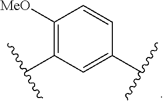 [SMILES: *c1ccc(OC)c(*)c1]

In some embodiments, -Cy- is

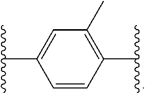 [SMILES: *c1ccc(*)c(C)c1]

In some embodiments, -Cy- is

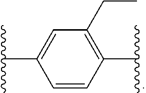 [SMILES: *c1ccc(*)c(CC)c1]

In some embodiments, -Cy- is

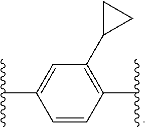 [SMILES: *c1ccc(*)c(C2CC2)c1]

In some embodiments, -Cy- is

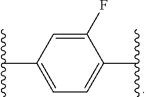 [SMILES: *c1ccc(*)c(F)c1]

In some embodiments, -Cy- is

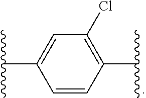 [SMILES: *c1ccc(*)c(Cl)c1]

In some embodiments, -Cy- is

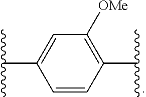 [SMILES: *c1ccc(*)c(OC)c1]

In some embodiments, -Cy- is

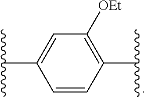 [SMILES: *c1ccc(*)c(OCC)c1]

In some embodiments, -Cy- is

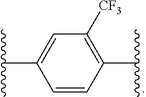 [SMILES: *c1ccc(*)c(C(F)(F)F)c1]

In some embodiments, -Cy- is

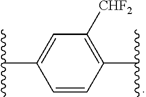 [SMILES: *c1ccc(*)c(C(F)F)c1]

In some embodiments, -Cy- is

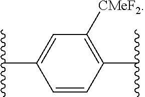

In some embodiments, -Cy- is

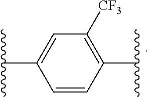 [SMILES: *c1ccc(*)c(C(F)(F)F)c1]

In some embodiments, -Cy- is

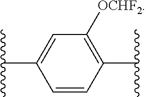 [SMILES: *c1ccc(*)c(OC(F)F)c1]

In some embodiments, -Cy- is

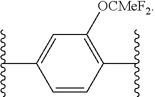

In some embodiments, -Cy- is

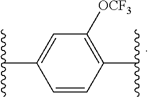 [SMILES: *c1ccc(*)c(OC(F)(F)F)c1]

In some embodiments, -Cy- is

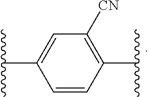 [SMILES: *c1ccc(*)c(C#N)c1]

In some embodiments, -Cy- is

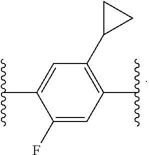 [SMILES: *c1cc(C2CC2)c(*)cc1F]

In some embodiments, -Cy- is

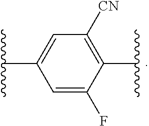 [SMILES: *c1cc(F)c(*)c(C#N)c1]

In some embodiments, -Cy- is

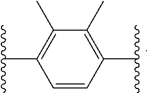 [SMILES: *c1ccc(*)c(C)c1C]

In some embodiments, -Cy- is

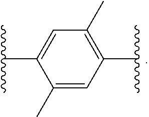 [SMILES: *c1cc(C)c(*)cc1C]

In some embodiments, -Cy- is

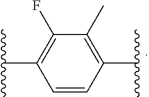 [SMILES: *c1ccc(*)c(F)c1C]

In some embodiments, -Cy- is

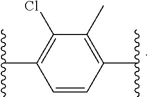 [SMILES: *c1ccc(*)c(Cl)c1C]

In some embodiments, -Cy- is

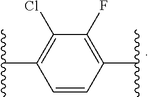 [SMILES: *c1ccc(*)c(Cl)c1F]

In some embodiments, -Cy- is

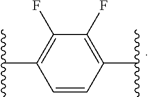 [SMILES: *c1ccc(*)c(F)c1F]

In some embodiments, -Cy- is

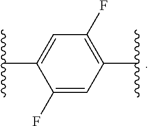 [SMILES: *c1cc(F)c(*)cc1F]

In some embodiments, -Cy- is

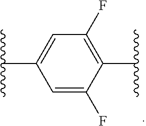 [SMILES: *c1cc(F)c(*)c(F)c1]

In some embodiments, -Cy- is

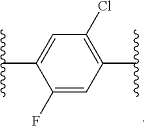 [SMILES: *c1cc(Cl)c(*)cc1F]

In some embodiments, -Cy- is

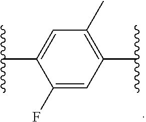 [SMILES: *c1cc(F)c(*)cc1C]

In some embodiments, -Cy- is

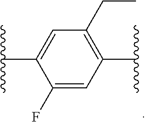 [SMILES: *c1cc(CC)c(*)cc1F]

In some embodiments, -Cy- is

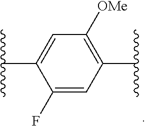 [SMILES: *c1cc(OC)c(*)cc1F]

In some embodiments, -Cy- is

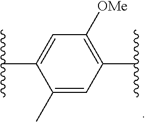 [SMILES: *c1cc(OC)c(*)cc1C]

In some embodiments, -Cy- is

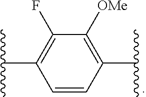 [SMILES: *c1ccc(*)c(OC)c1F]

In some embodiments, -Cy- is

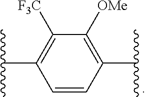 [SMILES: *c1ccc(*)c(C(F)(F)F)c1OC]

In some embodiments, -Cy- is

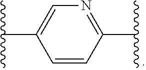 [SMILES: *c1ccc(*)nc1]

In some embodiments, -Cy- is

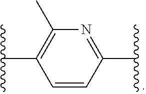 [SMILES: *c1ccc(*)c(C)n1]

In some embodiments, -Cy- is

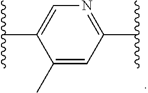 [SMILES: *c1cc(C)c(*)cn1]

In some embodiments, -Cy- is

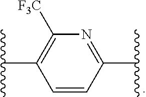 [SMILES: *c1ccc(*)c(C(F)(F)F)n1]

In some embodiments, -Cy- is

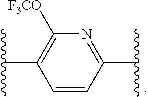 [SMILES: *c1ccc(*)c(OC(F)(F)F)n1]

In some embodiments, -Cy- is

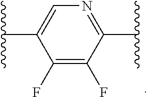 [SMILES: *c1cnc(*)c(F)c1F]

In some embodiments, -Cy- is

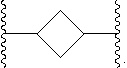 [SMILES: *C1CC(*)C1]

In some embodiments, -Cy- is

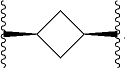 [SMILES: *[C@H]1C[C@@H](*)C1]

In some embodiments, -Cy- is

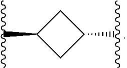 [SMILES: *[C@H]1C[C@H](*)C1]

In some embodiments, -Cy- is

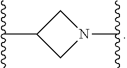 [SMILES: *C1CN(*)C1]

In some embodiments, -Cy- is

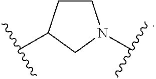 [SMILES: *C1CCN(*)C1]

In some embodiments, -Cy- is

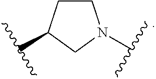 [SMILES: *[C@H]1CCN(*)C1]

In some embodiments, -Cy- is

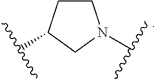 [SMILES: *[C@@H]1CCN(*)C1]

In some embodiments, -Cy- is

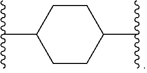 [SMILES: *C1CCC(*)CC1]

In some embodiments, -Cy- is

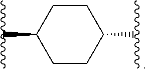 [SMILES: *[C@H]1CC[C@H](*)CC1]

In some embodiments, -Cy- is

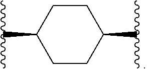 [SMILES: *[C@H]1CC[C@@H](*)CC1]

In some embodiments, -Cy- is

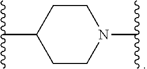 [SMILES: *C1CCN(*)CC1 ; "."]

In some embodiments, -Cy- is

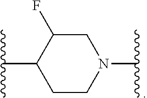 [SMILES: *C1CCN(*)CC1F ; "."]

In some embodiments, -Cy- is

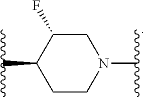 [SMILES: *[C@@H]1CCN(*)C[C@H]1F]

In some embodiments, -Cy- is

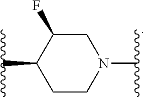 [SMILES: *[C@@H]1CCN(*)C[C@@H]1F]

In some embodiments, -Cy- is

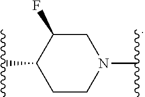 [SMILES: *[C@H]1CCN(*)C[C@@H]1F]

In some embodiments, -Cy- is

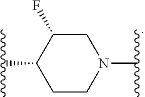 [SMILES: *[C@H]1CCN(*)C[C@H]1F]

In some embodiments, -Cy- is

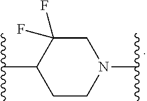 [SMILES: *C1CCN(*)CC1(F)F]

In some embodiments, -Cy- is

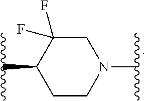 [SMILES: *[C@@H]1CCN(*)CC1(F)F]

In some embodiments, -Cy- is

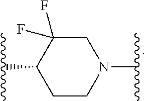 [SMILES: *[C@H]1CCN(*)CC1(F)F]

In some embodiments, -Cy- is

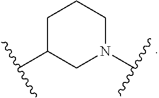 [SMILES: *C1CCCN(*)C1]

In some embodiments, -Cy- is

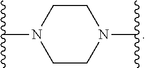 [SMILES: *N1CCN(*)CC1]

In some embodiments, -Cy- is

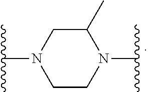 [SMILES: *N1CCN(*)C(C)C1]

In some embodiments, -Cy- is

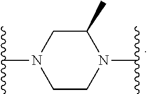 [SMILES: *N1CCN(*)[C@H](C)C1]

In some embodiments, -Cy- is

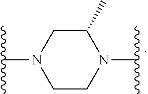 [SMILES: *N1CCN(*)[C@@H](C)C1]

In some embodiments, -Cy- is

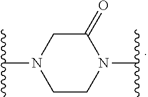 [SMILES: *N1CCN(*)C(=O)C1]

In some embodiments, -Cy- is

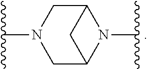 [SMILES: *N1CC2CC(C1)N2*]

In some embodiments, -Cy- is

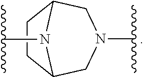 [SMILES: *N1CC2CCC(C1)N2*]

In some embodiments, -Cy- is

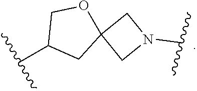 [SMILES: *C1COC2(C1)CN(*)C2]

In some embodiments, -Cy- is

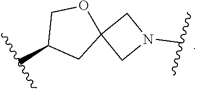 [SMILES: *[C@H]1COC2(C1)CN(*)C2]

In some embodiments, -Cy- is

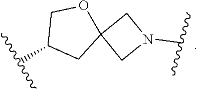 [SMILES: *[C@@H]1COC2(C1)CN(*)C2]

In some embodiments, -Cy- is

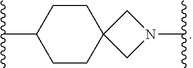 [SMILES: *C1CCC2(CC1)CN(*)C2]

In some embodiments, -Cy- is

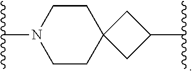 [SMILES: *C1CC2(CCN(*)CC2)C1]

In some embodiments, -Cy- is

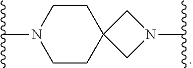 [SMILES: *N1CCC2(CC1)CN(*)C2]

In some embodiments, -Cy- is

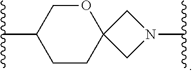 [SMILES: *C1CCC2(CN(*)C2)OC1]

In some embodiments, -Cy- is

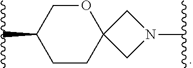 [SMILES: *[C@@H]1CCC2(CN(*)C2)OC1]

In some embodiments, -Cy- is

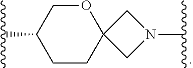 [SMILES: *[C@H]1CCC2(CN(*)C2)OC1]

In some embodiments, -Cy- is

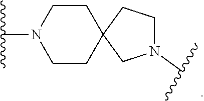 [SMILES: *N1CCC2(CC1)CCN(*)C2]

In some embodiments, -Cy- is

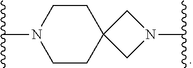 [SMILES: *N1CCC2(CC1)CN(*)C2]

In some embodiments, -Cy- is

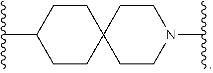 [SMILES: *C1CCC2(CC1)CCN(*)CC2]

In some embodiments, -Cy- is

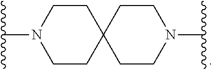 [SMILES: *N1CCC2(CC1)CCN(*)CC2]

In some embodiments, -Cy- is

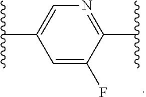 [SMILES: *c1cnc(*)c(F)c1]

In some embodiments, -Cy- is selected from those depicted in Table 1A, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1A, below.

In some embodiments, L is

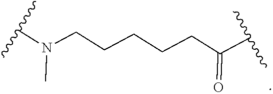 [SMILES: *C(=O)CCCCCN(*)C]

In some embodiments, L is

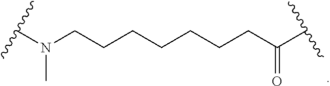 [SMILES: *C(=O)CCCCCCCN(*)C]

In some embodiments, L is

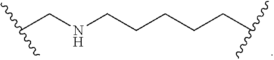 [SMILES: *CCCCCNC*]

In some embodiments, L is

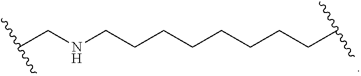 [SMILES: *CCCCCCCCNC*]

In some embodiments, L is

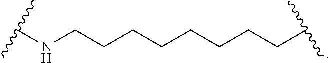 [SMILES: *CCCCCCCCN*]

In some embodiments, L is

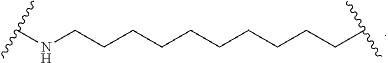 [SMILES: *CCCCCCCCCCN*]

In some embodiments, L is

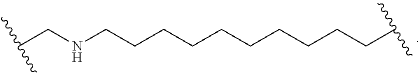 [SMILES: *CCCCCCCCCCNC*]

In some embodiments, L is

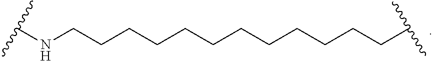 [SMILES: *CCCCCCCCCCCCN*]

In some embodiments, L is

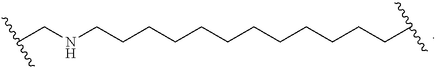 [SMILES: *CCCCCCCCCCCCNC*]

In some embodiments, L is

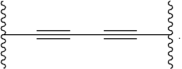 [SMILES: *C#CC#C*]

In some embodiments, L is

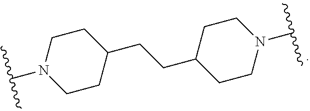 [SMILES: *N1CCC(CCC2CCN(*)CC2)CC1]

In some embodiments, L is

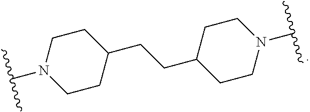 [SMILES: *N1CCC(CCC2CCN(*)CC2)CC1]

In some embodiments, L is

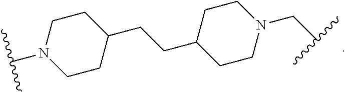 [SMILES: *CN1CCC(CCC2CCN(*)CC2)CC1]

In some embodiments, L is

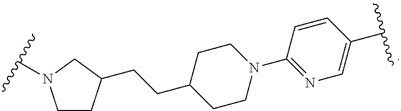 [SMILES: *c1ccc(N2CCC(CCC3CCN(*)C3)CC2)nc1]

In some embodiments, L is

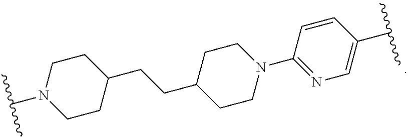 [SMILES: *c1ccc(N2CCC(CCC3CCN(*)CC3)CC2)nc1]

In some embodiments, L is

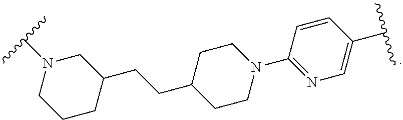 [SMILES: *c1ccc(N2CCC(CCC3CCCN(*)C3)CC2)nc1]

In some embodiments, L is

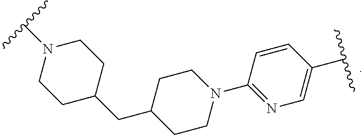 [SMILES: *c1ccc(N2CCC(CC3CCN(*)CC3)CC2)nc1]

In some embodiments, L is

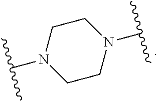 [SMILES: *N1CCN(*)CC1]

In some embodiments, L is

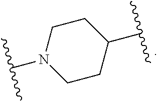 [SMILES: *C1CCN(*)CC1]

In some embodiments, L is

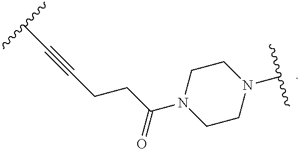 [SMILES: *C#CCCC(=O)N1CCN(*)CC1]

In some embodiments, L is

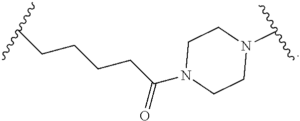 [SMILES: *CCCCC(=O)N1CCN(*)CC1]

In some embodiments, L is

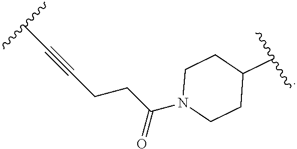 [SMILES: *C#CCCC(=O)N1CCC(*)CC1]

In some embodiments, L is

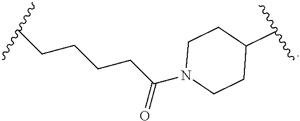 [SMILES: *CCCCC(=O)N1CCC(*)CC1]

In some embodiments, L is

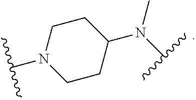 [SMILES: *N1CCC(N(*)C)CC1]

In some embodiments, L is

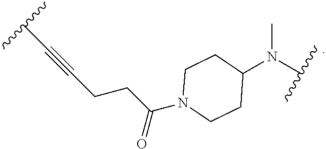 [SMILES: *C#CCCC(=O)N1CCC(N(*)C)CC1]

In some embodiments, L is

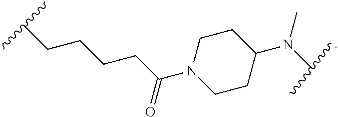 [SMILES: *CCCCC(=O)N1CCC(N(*)C)CC1]

In some embodiments, L is

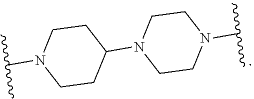 [SMILES: *N1CCC(N2CCN(*)CC2)CC1]

In some embodiments, L is

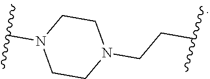 [SMILES: *CCN1CCN(*)CC1]

In some embodiments, L is

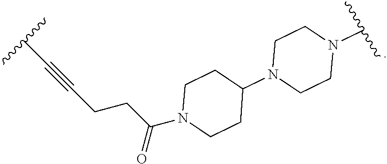 [SMILES: *C#CCCC(=O)N1CCC(N2CCN(*)CC2)CC1]

In some embodiments, L is

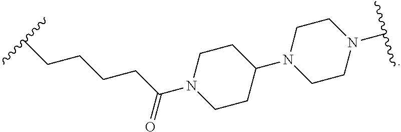 [SMILES: *CCCCC(=O)N1CCC(N2CCN(*)CC2)CC1]

In some embodiments, L is

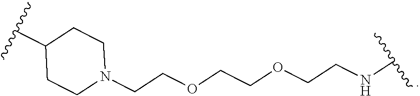 [SMILES: *NCCOCCOCCN1CCC(*)CC1]

In some embodiments, L is

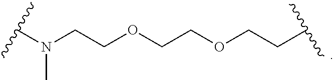 [SMILES: *CCOCCOCCN(*)C]

In some embodiments, L is

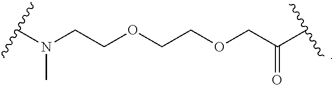 [SMILES: *C(=O)COCCOCCN(*)C]

In some embodiments, L is

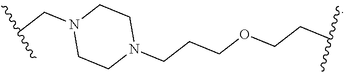 [SMILES: *CCOCCCN1CCN(C*)CC1]

In some embodiments, L is

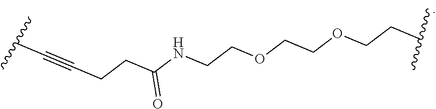 [SMILES: *C#CCCC(=O)NCCOCCOCC*]

In some embodiments, L is

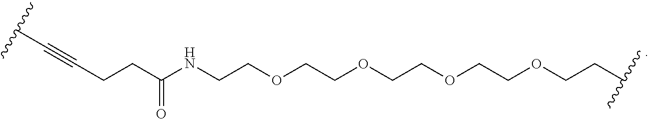 [SMILES: *C#CCCC(=O)NCCOCCOCCOCCOCC*]

In some embodiments, L is

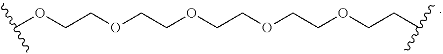 [SMILES: *CCOCCOCCOCCOCCO*]

In some embodiments, L is

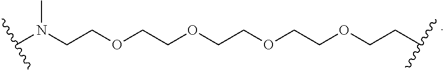 [SMILES: *CCOCCOCCOCCOCCN(*)C]

In some embodiments, L is

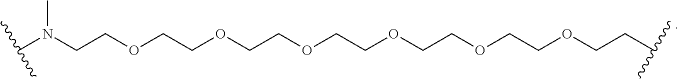 [SMILES: *CCOCCOCCOCCOCCOCCOCCN(*)C]

In some embodiments, L is

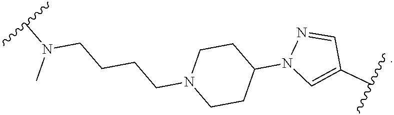 [SMILES: *c1cnn(C2CCN(CCCCN(*)C)CC2)c1]

In some embodiments, L is

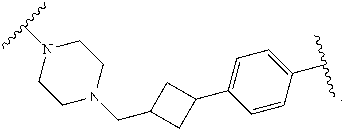 [SMILES: *c1ccc(C2CC(CN3CCN(*)CC3)C2)cc1]

In some embodiments, L is

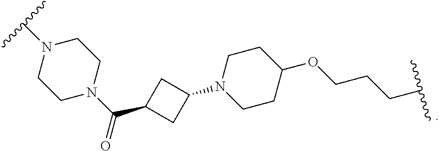 [SMILES: *CCCOC1CCN([C@H]2C[C@H](C(=O)N3CCN(*)CC3)C2)CC1]

In some embodiments, L is

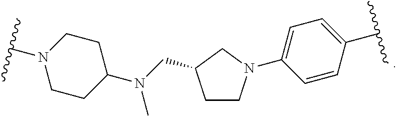 [SMILES: *c1ccc(N2CC[C@H](CN(C)C3CCN(*)CC3)C2)cc1]

In some embodiments, L is

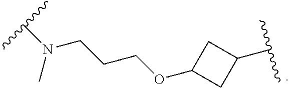 [SMILES: *C1CC(OCCCN(*)C)C1]

In some embodiments, L is

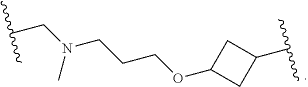 [SMILES: *CN(C)CCCOC1CC(*)C1]

In some embodiments, L is

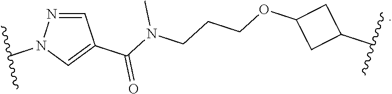 [SMILES: *C1CC(OCCCN(C)C(=O)c2cnn(*)c2)C1]

In some embodiments, L is

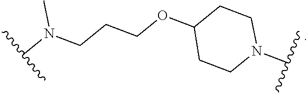 [SMILES: *N(C)CCCOC1CCN(*)CC1]

In some embodiments, L is

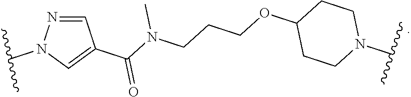 [SMILES: *N1CCC(OCCCN(C)C(=O)c2cnn(*)c2)CC1]

In some embodiments, L is

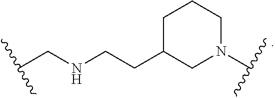 [SMILES: *CNCCC1CCCN(*)C1]

In some embodiments, L is

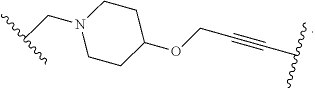 [SMILES: *C#CCOC1CCN(C*)CC1]

In some embodiments, L is

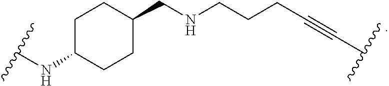 [SMILES: *C#CCCCNC[C@H]1CC[C@H](N*)CC1]

In some embodiments, L is

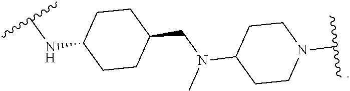 [SMILES: *N[C@H]1CC[C@H](CN(C)C2CCN(*)CC2)CC1]

In some embodiments, L is

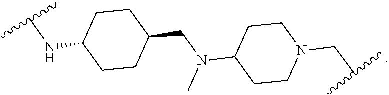 [SMILES: *CN1CCC(N(C)C[C@H]2CC[C@H](N*)CC2)CC1]

In some embodiments, L is

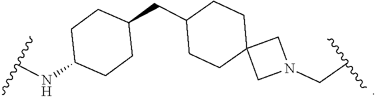 [SMILES: *CN1CC2(CCC(C[C@H]3CC[C@H](N*)CC3)CC2)C1]

In some embodiments, L is

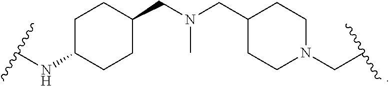 [SMILES: *CN1CCC(CN(C)C[C@H]2CC[C@H](N*)CC2)CC1]

In some embodiments, L is

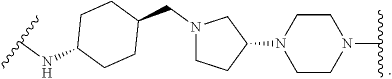 [SMILES: *N[C@H]1CC[C@H](CN2CC[C@@H](N3CCN(*)CC3)C2)CC1]

In some embodiments, L is

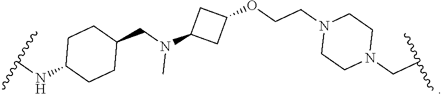 [SMILES: *CN1CCN(CCO[C@H]2C[C@H](N(C)C[C@H]3CC[C@H](N*)CC3)C2)CC1]

In some embodiments, L is

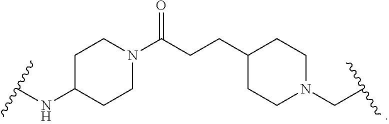 [SMILES: *CN1CCC(CCC(=O)N2CCC(N*)CC2)CC1]

In some embodiments, L is

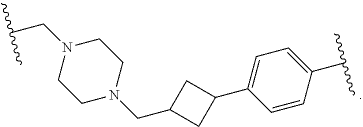 [SMILES: *CN1CCN(CC2CC(c3ccc(*)cc3)C2)CC1]

In some embodiments, L is

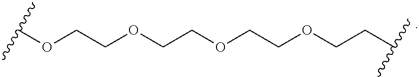 [SMILES: *CCOCCOCCOCCO*]

In some embodiments, L is

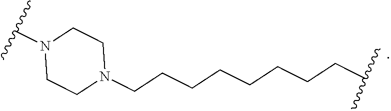 [SMILES: *CCCCCCCCN1CCN(*)CC1]

In some embodiments, L is

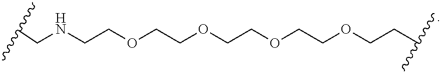 [SMILES: *CCOCCOCCOCCOCCNC*]

In some embodiments, L is

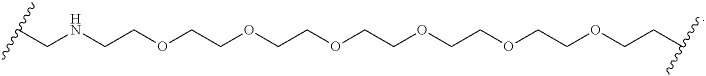 [SMILES: *CCOCCOCCOCCOCCOCCOCCNC*]

In some embodiments, L is

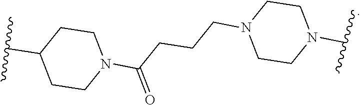 [SMILES: *C1CCN(C(=O)CCCN2CCN(*)CC2)CC1]

In some embodiments, L is

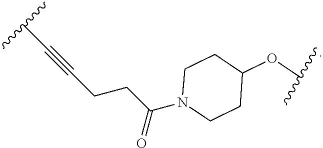 [SMILES: *C#CCCC(=O)N1CCC(O*)CC1]

In some embodiments, L is

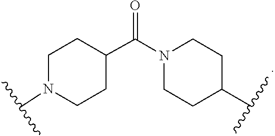 [SMILES: *C1CCN(C(=O)C2CCN(*)CC2)CC1]

In some embodiments, L is

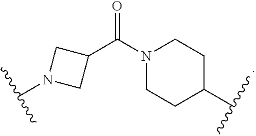 [SMILES: *C1CCN(C(=O)C2CN(*)C2)CC1]

In some embodiments, L is

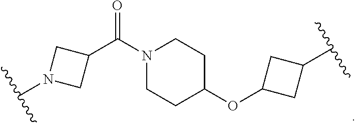 [SMILES: *C1CC(OC2CCN(C(=O)C3CN(*)C3)CC2)C1]

In some embodiments, L is

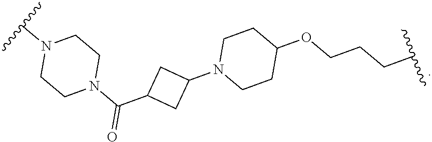 [SMILES: *CCCOC1CCN(C2CC(C(=O)N3CCN(*)CC3)C2)CC1]

In some embodiments, L is

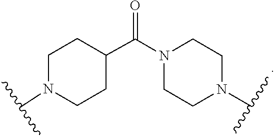 [SMILES: *N1CCC(C(=O)N2CCN(*)CC2)CC1]

In some embodiments, L is

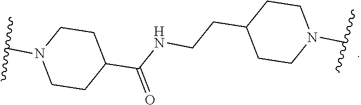 [SMILES: *N1CCC(CCNC(=O)C2CCN(*)CC2)CC1]

In some embodiments, L is

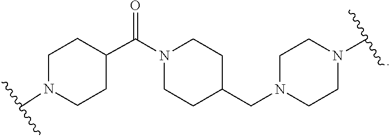 [SMILES: *N1CCC(C(=O)N2CCC(CN3CCN(*)CC3)CC2)CC1]

In some embodiments, L is

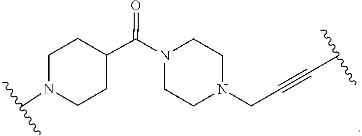 [SMILES: *C#CCN1CCN(C(=O)C2CCN(*)CC2)CC1]

In some embodiments, L is

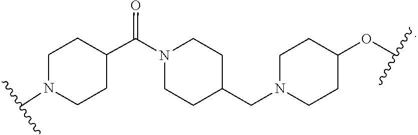 [SMILES: *OC1CCN(CC2CCN(C(=O)C3CCN(*)CC3)CC2)CC1]

In some embodiments, L is

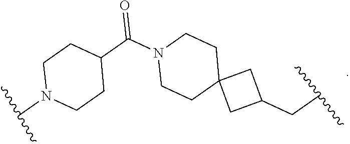 [SMILES: *CC1CC2(CCN(C(=O)C3CCN(*)CC3)CC2)C1]

In some embodiments, L is

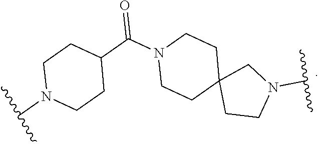 [SMILES: *N1CCC(C(=O)N2CCC3(CCN(*)C3)CC2)CC1]

In some embodiments, L is

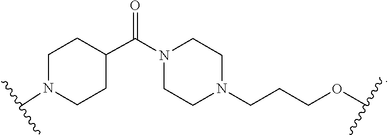 [SMILES: *OCCCN1CCN(C(=O)C2CCN(*)CC2)CC1]

In some embodiments, L is

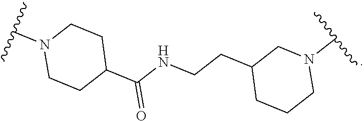 [SMILES: *N1CCC(C(=O)NCCC2CCCN(*)C2)CC1]

In some embodiments, L is

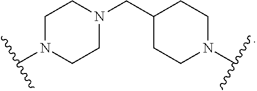 [SMILES: *N1CCC(CN2CCN(*)CC2)CC1]

In some embodiments, L is

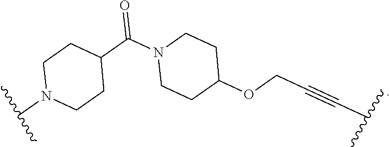 [SMILES: *C#CCOC1CCN(C(=O)C2CCN(*)CC2)CC1]

In some embodiments, L is

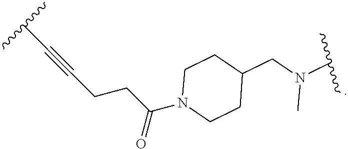 [SMILES: *C#CCCC(=O)N1CCC(CN(*)C)CC1]

In some embodiments, L is

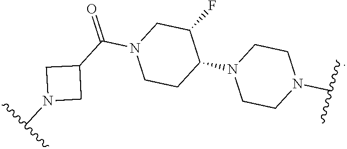 [SMILES: *N1CCN([C@@H]2CCN(C(=O)C3CN(*)C3)C[C@@H]2F)CC1]

In some embodiments, L is

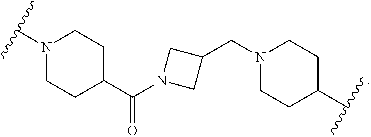 [SMILES: *C1CCN(CC2CN(C(=O)C3CCN(*)CC3)C2)CC1]

In some embodiments, L is

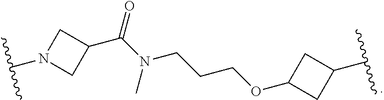 [SMILES: *C1CC(OCCCN(C)C(=O)C2CN(*)C2)C1]

In some embodiments, L is

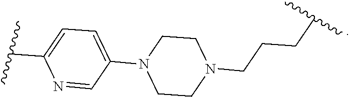 [SMILES: *CCCN1CCN(c2ccc(*)nc2)CC1]

In some embodiments, L is

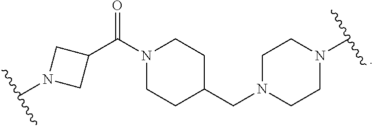 [SMILES: *N1CCN(CC2CCN(C(=O)C3CN(*)C3)CC2)CC1]

In some embodiments, L is

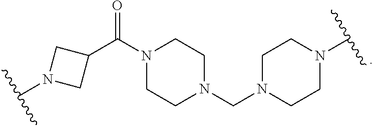 [SMILES: *N1CCN(CN2CCN(C(=O)C3CN(*)C3)CC2)CC1]

In some embodiments, L is

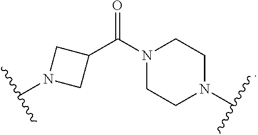 [SMILES: *N1CCN(C(=O)C2CN(*)C2)CC1]

In some embodiments, L is

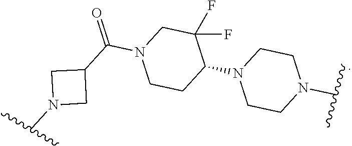 [SMILES: *N1CCN([C@@H]2CCN(C(=O)C3CN(*)C3)CC2(F)F)CC1]

In some embodiments, L is

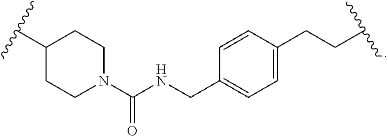 [SMILES: *CCc1ccc(CNC(=O)N2CCC(*)CC2)cc1]

In some embodiments, L is

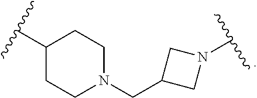 [SMILES: *C1CCN(CC2CN(*)C2)CC1]

In some embodiments, L is

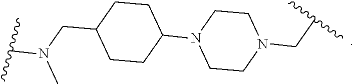 [SMILES: *CN1CCN(C2CCC(CN(*)C)CC2)CC1]

In some embodiments, L is

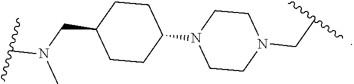 [SMILES: *CN1CCN([C@H]2CC[C@H](CN(*)C)CC2)CC1]

In some embodiments, L is

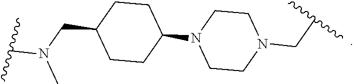 [SMILES: *CN1CCN([C@H]2CC[C@@H](CN(*)C)CC2)CC1]

In some embodiments, L is

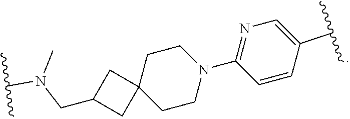 [SMILES: *c1ccc(N2CCC3(CC2)CC(CN(*)C)C3)nc1]

In some embodiments, L is

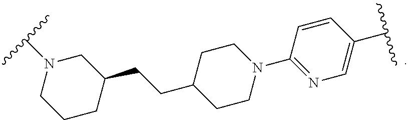 [SMILES: *c1ccc(N2CCC(CC[C@H]3CCCN(*)C3)CC2)nc1]

In some embodiments, L is

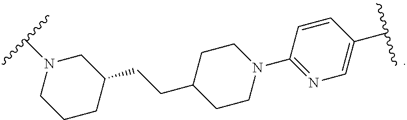 [SMILES: *c1ccc(N2CCC(CC[C@@H]3CCCN(*)C3)CC2)nc1]

In some embodiments, L is

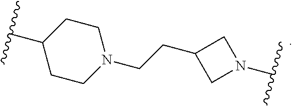 [SMILES: *C1CCN(CCC2CN(*)C2)CC1]

In some embodiments, L is

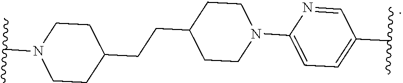 [SMILES: *c1ccc(N2CCC(CCC3CCN(*)CC3)CC2)nc1]

In some embodiments, L is

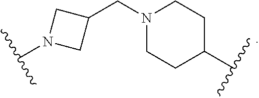 [SMILES: *C1CCN(CC2CN(*)C2)CC1]

In some embodiments, L is

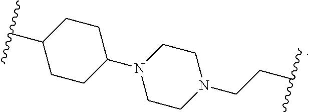 [SMILES: *CCN1CCN(C2CCC(*)CC2)CC1]

In some embodiments, L is

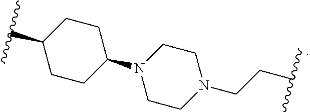 [SMILES: *CCN1CCN([C@H]2CC[C@@H](*)CC2)CC1]

In some embodiments, L is

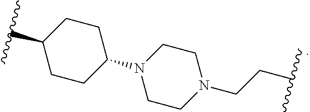 [SMILES: *CCN1CCN([C@H]2CC[C@H](*)CC2)CC1]

In some embodiments, L is

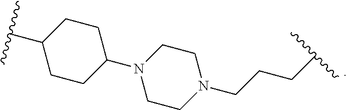 [SMILES: *CCCN1CCN(C2CCC(*)CC2)CC1]

In some embodiments, L is

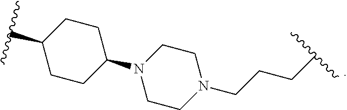 [SMILES: *CCCN1CCN([C@H]2CC[C@@H](*)CC2)CC1]

In some embodiments, L is

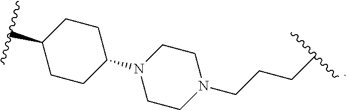 [SMILES: *CCCN1CCN([C@H]2CC[C@H](*)CC2)CC1]

In some embodiments, L is

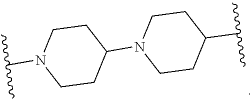 [SMILES: *C1CCN(C2CCN(*)CC2)CC1]

In some embodiments, L is

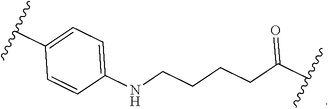 [SMILES: *C(=O)CCCCNc1ccc(*)cc1]

In some embodiments, L is

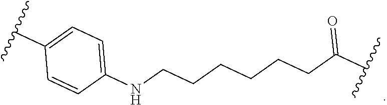 [SMILES: *C(=O)CCCCCCNc1ccc(*)cc1]

In some embodiments, L is

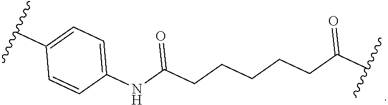 [SMILES: *C(=O)CCCCCC(=O)Nc1ccc(*)cc1]

In some embodiments, L is

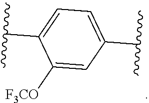 [SMILES: *c1ccc(*)c(OC(F)(F)F)c1]

In some embodiments, L is

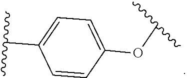 [SMILES: *Oc1ccc(*)cc1]

In some embodiments, L is

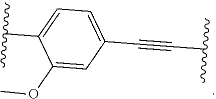 [SMILES: *C#Cc1ccc(*)c(OC)c1]

In some embodiments, L is

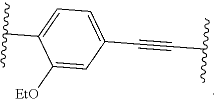 [SMILES: *C#Cc1ccc(*)c(OCC)c1]

In some embodiments, L is

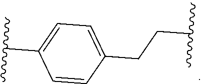 [SMILES: *CCc1ccc(*)cc1]

In some embodiments, L is

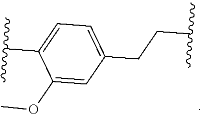 [SMILES: *CCc1ccc(*)c(OC)c1]

In some embodiments, L is

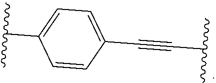 [SMILES: *C#Cc1ccc(*)cc1]

In some embodiments, L is

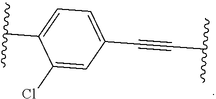 [SMILES: *C#Cc1ccc(*)c(Cl)c1]

In some embodiments, L is

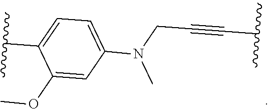 [SMILES: *C#CCN(C)c1ccc(*)c(OC)c1]

In some embodiments, L is

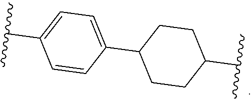 [SMILES: *c1ccc(C2CCC(*)CC2)cc1]

In some embodiments, L is

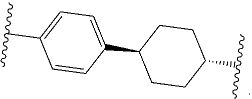 [SMILES: *c1ccc([C@H]2CC[C@H](*)CC2)cc1]

In some embodiments, L is

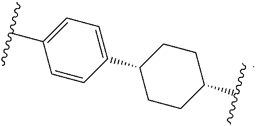 [SMILES: *c1ccc([C@H]2CC[C@@H](*)CC2)cc1]

In some embodiments, L is

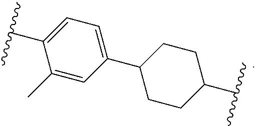 [SMILES: *c1ccc(C2CCC(*)CC2)cc1C]

In some embodiments, L is

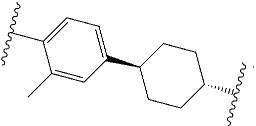 [SMILES: *c1ccc([C@H]2CC[C@H](*)CC2)cc1C]

In some embodiments, L is

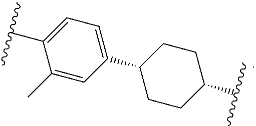 [SMILES: *c1ccc([C@H]2CC[C@@H](*)CC2)cc1C]

In some embodiments, L is

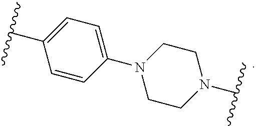 [SMILES: *c1ccc(N2CCN(*)CC2)cc1]

In some embodiments, L is

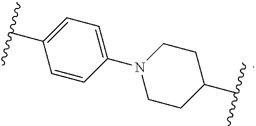 [SMILES: *c1ccc(N2CCC(*)CC2)cc1]

In some embodiments, L is

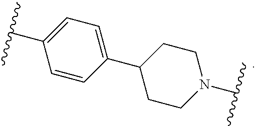 [SMILES: *c1ccc(C2CCN(*)CC2)cc1]

In some embodiments, L is

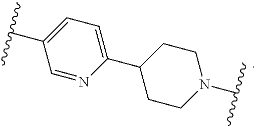 [SMILES: *c1ccc(C2CCN(*)CC2)nc1]

In some embodiments, L is

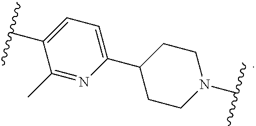 [SMILES: *c1ccc(C2CCN(*)CC2)nc1C]

In some embodiments, L is

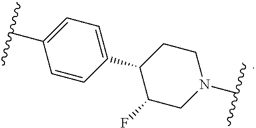 [SMILES: *c1ccc([C@@H]2CCN(*)C[C@@H]2F)cc1]

In some embodiments, L is

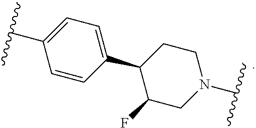 [SMILES: *c1ccc([C@H]2CCN(*)C[C@H]2F)cc1]

In some embodiments,

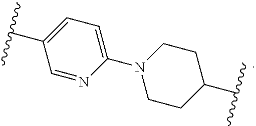 [SMILES: *c1ccc(N2CCC(*)CC2)nc1]

In some embodiments, L is

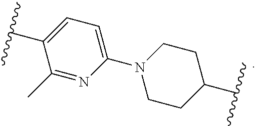 [SMILES: *c1ccc(N2CCC(*)CC2)nc1C]

### L is

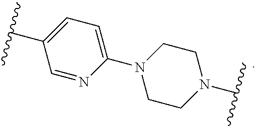 [SMILES: *c1ccc(N2CCN(*)CC2)nc1]

In some embodiments, L is

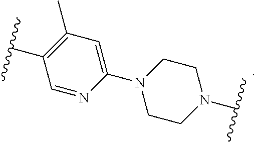 [SMILES: *c1cnc(N2CCN(*)CC2)cc1C]

In some embodiments, L is

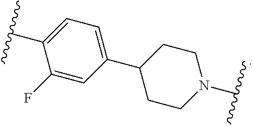 [SMILES: *c1ccc(C2CCN(*)CC2)cc1F]

In some embodiments, L is

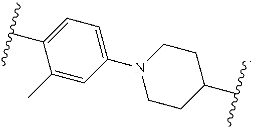 [SMILES: *c1ccc(N2CCC(*)CC2)cc1C]

In some embodiments, L is

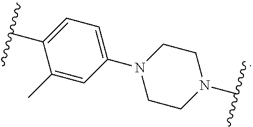 [SMILES: *c1ccc(N2CCN(*)CC2)cc1C]

In some embodiments, L is

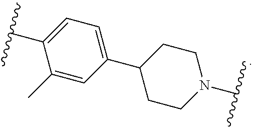 [SMILES: *c1ccc(C2CCN(*)CC2)cc1C]

In some embodiments, L is

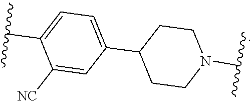 [SMILES: *c1ccc(C2CCN(*)CC2)cc1C#N]

In some embodiments, L is

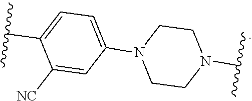 [SMILES: *c1ccc(N2CCN(*)CC2)cc1C#N]

In some embodiments, L is

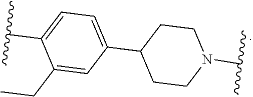 [SMILES: *c1ccc(C2CCN(*)CC2)cc1CC]

In some embodiments, L is

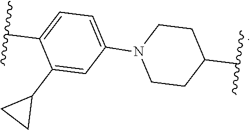 [SMILES: *c1ccc(N2CCC(*)CC2)cc1C1CC1]

In some embodiments, L is

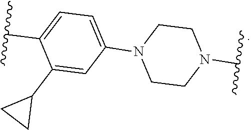 [SMILES: *c1ccc(N2CCN(*)CC2)cc1C1CC1]

In some embodiments, L is

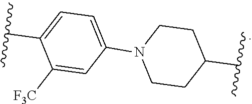 [SMILES: *c1ccc(N2CCC(*)CC2)cc1C(F)(F)F]

In some embodiments, L is

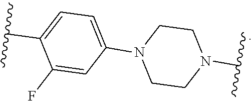 [SMILES: *c1ccc(N2CCN(*)CC2)cc1F]

In some embodiments, L is

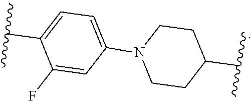 [SMILES: *c1ccc(N2CCC(*)CC2)cc1F]

In some embodiments, L is

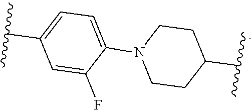 [SMILES: *c1ccc(N2CCC(*)CC2)c(F)c1]

In some embodiments, L is

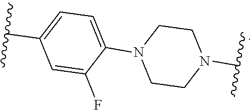 [SMILES: *c1ccc(N2CCN(*)CC2)c(F)c1]

In some embodiments, L is

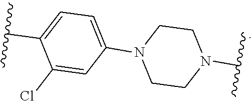 [SMILES: *c1ccc(N2CCN(*)CC2)cc1Cl]

In some embodiments, L is

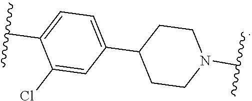 [SMILES: *c1ccc(C2CCN(*)CC2)cc1Cl]

In some embodiments, L is

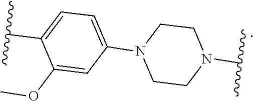 [SMILES: *c1ccc(N2CCN(*)CC2)cc1OC]

In some embodiments, L is

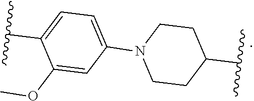 [SMILES: *c1ccc(N2CCC(*)CC2)cc1OC]

In some embodiments, L is

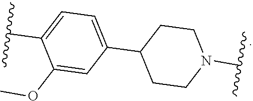 [SMILES: *c1ccc(C2CCN(*)CC2)cc1OC]

In some embodiments, L is

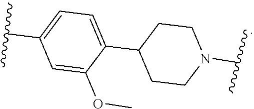 [SMILES: *c1ccc(C2CCN(*)CC2)c(OC)c1]

In some embodiments, L is

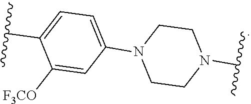 [SMILES: *c1ccc(N2CCN(*)CC2)cc1OC(F)(F)F]

In some embodiments, L is

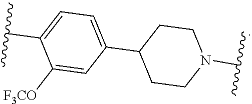 [SMILES: *c1ccc(C2CCN(*)CC2)cc1OC(F)(F)F]

In some embodiments, L is

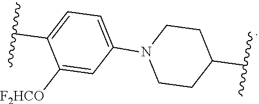 [SMILES: *c1ccc(N2CCC(*)CC2)cc1OC(F)F]

In some embodiments, L is

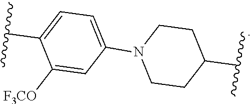 [SMILES: *c1ccc(N2CCC(*)CC2)cc1OC(F)(F)F]

In some embodiments, L is

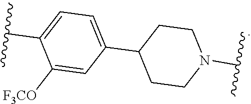 [SMILES: *c1ccc(C2CCN(*)CC2)cc1OC(F)(F)F]

In some embodiments, L is

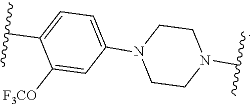 [SMILES: *c1ccc(N2CCN(*)CC2)cc1OC(F)(F)F]

In some embodiments, L is

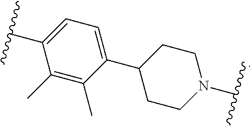 [SMILES: *c1ccc(C2CCN(*)CC2)c(C)c1C]

In some embodiments, L is

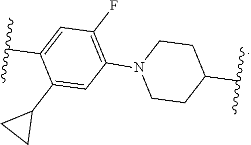 [SMILES: *c1cc(F)c(N2CCC(*)CC2)cc1C1CC1]

In some embodiments, L is

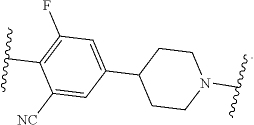 [SMILES: *c1c(F)cc(C2CCN(*)CC2)cc1C#N]

In some embodiments, L is

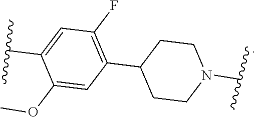 [SMILES: *c1cc(F)c(C2CCN(*)CC2)cc1OC]

In some embodiments, L is

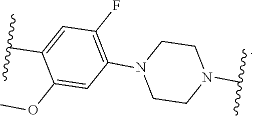 [SMILES: *c1cc(F)c(N2CCN(*)CC2)cc1OC]

In some embodiments, L is

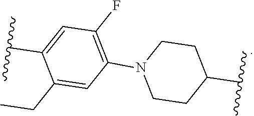 [SMILES: *c1cc(F)c(N2CCC(*)CC2)cc1CC]

In some embodiments, L is

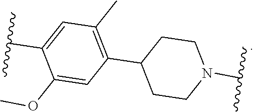 [SMILES: *c1cc(C)c(C2CCN(*)CC2)cc1OC]

In some embodiments, L is

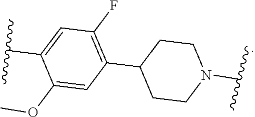 [SMILES: *c1cc(F)c(C2CCN(*)CC2)cc1OC]

In some embodiments, L is

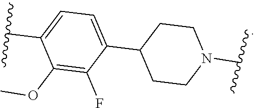 [SMILES: *c1ccc(C2CCN(*)CC2)c(F)c1OC]

In some embodiments, L is

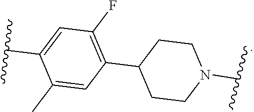 [SMILES: *c1cc(F)c(C2CCN(*)CC2)cc1C]

In some embodiments, L is

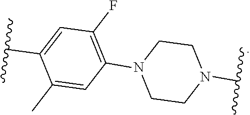 [SMILES: *c1cc(F)c(N2CCN(*)CC2)cc1C]

In some embodiments, L is

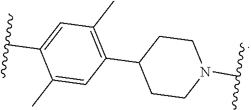 [SMILES: *c1cc(C)c(C2CCN(*)CC2)cc1C]

In some embodiments, L is

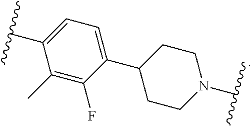 [SMILES: *c1ccc(C2CCN(*)CC2)c(F)c1C]

In some embodiments, L is

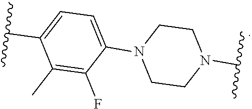 [SMILES: *c1ccc(N2CCN(*)CC2)c(F)c1C]

In some embodiments, L is

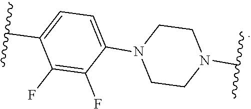 [SMILES: *c1ccc(N2CCN(*)CC2)c(F)c1F]

In some embodiments, L is

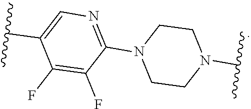 [SMILES: *c1cnc(N2CCN(*)CC2)c(F)c1F]

In some embodiments, L is

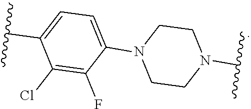 [SMILES: *c1ccc(N2CCN(*)CC2)c(F)c1Cl]

In some embodiments, L is

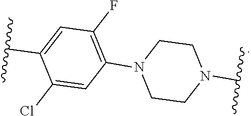 [SMILES: *c1cc(F)c(N2CCN(*)CC2)cc1Cl]

In some embodiments, L is

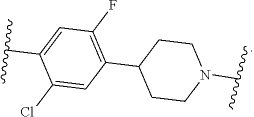 [SMILES: *c1cc(F)c(C2CCN(*)CC2)cc1Cl]

In some embodiments, L is

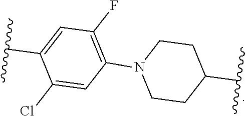 [SMILES: *c1cc(F)c(N2CCC(*)CC2)cc1Cl]

In some embodiments, L is

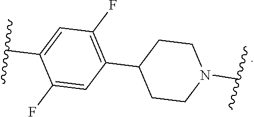 [SMILES: *c1cc(F)c(C2CCN(*)CC2)cc1F]

In some embodiments, L is

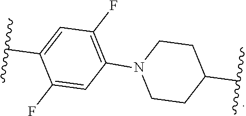 [SMILES: *c1cc(F)c(N2CCC(*)CC2)cc1F]

In some embodiments, L is

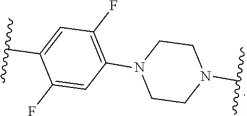 [SMILES: *c1cc(F)c(N2CCN(*)CC2)cc1F]

In some embodiments, L is

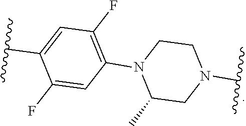 [SMILES: *c1cc(F)c(N2CCN(*)C[C@@H]2C)cc1F]

In some embodiments, L is

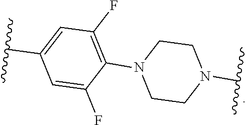 [SMILES: *c1cc(F)c(N2CCN(*)CC2)c(F)c1]

In some embodiments, L is

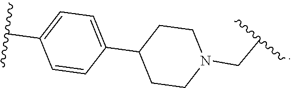 [SMILES: *CN1CCC(c2ccc(*)cc2)CC1]

In some embodiments, L is

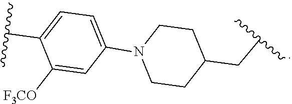 [SMILES: *CC1CCN(c2ccc(*)c(OC(F)(F)F)c2)CC1]

In some embodiments, L is

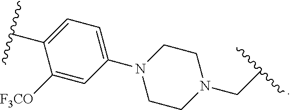 [SMILES: *CN1CCN(c2ccc(*)c(OC(F)(F)F)c2)CC1]

In some embodiments, L is

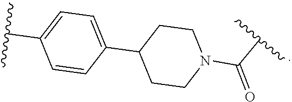 [SMILES: *C(=O)N1CCC(c2ccc(*)cc2)CC1]

In some embodiments, L is

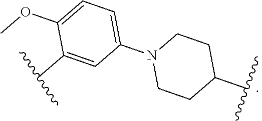 [SMILES: *c1cc(N2CCC(*)CC2)ccc1OC]

In some embodiments, L is

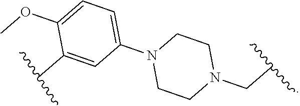 [SMILES: *CN1CCN(c2ccc(OC)c(*)c2)CC1]

In some embodiments, L is

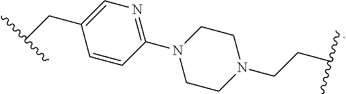 [SMILES: *CCN1CCN(c2ccc(C*)cn2)CC1]

In some embodiments, L is

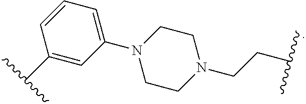 [SMILES: *CCN1CCN(c2cccc(*)c2)CC1]

In some embodiments, L is

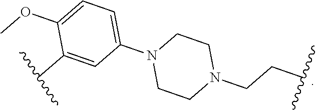 [SMILES: *CCN1CCN(c2ccc(OC)c(*)c2)CC1]

In some embodiments, L is

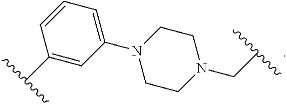 [SMILES: *CN1CCN(c2cccc(*)c2)CC1]

In some embodiments, L is

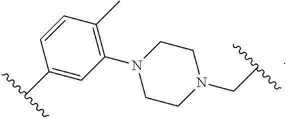 [SMILES: *CN1CCN(c2cc(*)ccc2C)CC1]

In some embodiments, L is

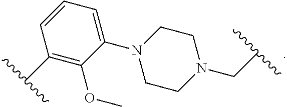 [SMILES: *CN1CCN(c2cccc(*)c2OC)CC1]

In some embodiments, L is

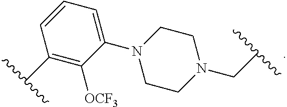 [SMILES: *CN1CCN(c2cccc(*)c2OC(F)(F)F)CC1]

In some embodiments, L is

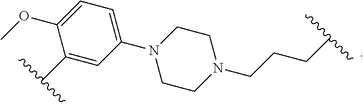 [SMILES: *CCCN1CCN(c2ccc(OC)c(*)c2)CC1]

In some embodiments, L is

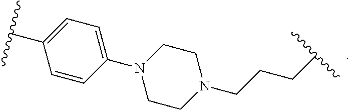 [SMILES: *CCCN1CCN(c2ccc(*)cc2)CC1]

In some embodiments, L is

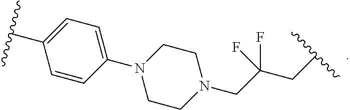 [SMILES: *CC(F)(F)CN1CCN(c2ccc(*)cc2)CC1]

In some embodiments, L is

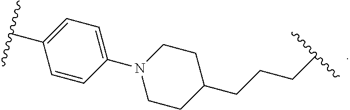 [SMILES: *CCCC1CCN(c2ccc(*)cc2)CC1]

In some embodiments, L is

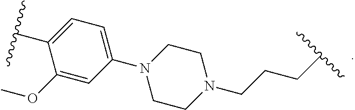 [SMILES: *CCCN1CCN(c2ccc(*)c(OC)c2)CC1]

In some embodiments, L is

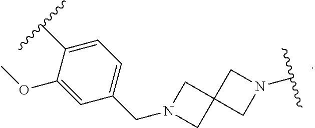 [SMILES: *c1ccc(CN2CC3(CN(*)C3)C2)cc1OC]

In some embodiments, L is

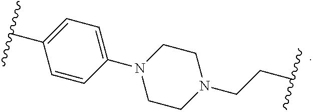 [SMILES: *CCN1CCN(c2ccc(*)cc2)CC1]

In some embodiments, L is

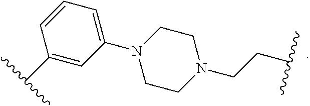 [SMILES: *CCN1CCN(c2cccc(*)c2)CC1]

In some embodiments, L is

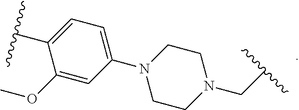 [SMILES: *CN1CCN(c2ccc(*)c(OC)c2)CC1]

In some embodiments, L is

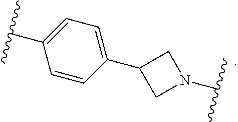 [SMILES: *c1ccc(C2CN(*)C2)cc1]

In some embodiments, L is

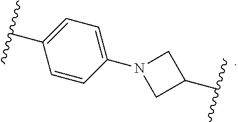 [SMILES: *c1ccc(N2CC(*)C2)cc1]

In some embodiments, L is

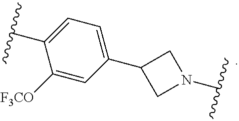 [SMILES: *c1ccc(C2CN(*)C2)cc1OC(F)(F)F]

In some embodiments, L is

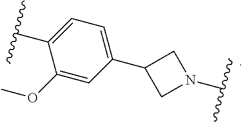 [SMILES: *c1ccc(C2CN(*)C2)cc1OC]

In some embodiments, L is

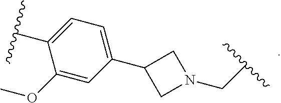 [SMILES: *CN1CC(c2ccc(*)c(OC)c2)C1]

In some embodiments, L is

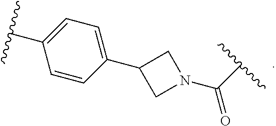 [SMILES: *C(=O)N1CC(c2ccc(*)cc2)C1]

In some embodiments, L is

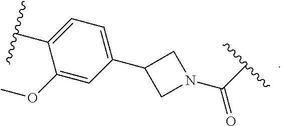 [SMILES: *C(=O)N1CC(c2ccc(*)c(OC)c2)C1]

In some embodiments, L is

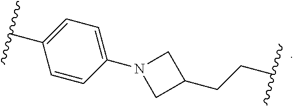 [SMILES: *CCC1CN(c2ccc(*)cc2)C1]

In some embodiments, L is

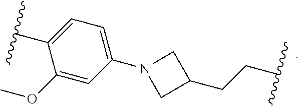 [SMILES: *CCC1CN(c2ccc(*)c(OC)c2)C1]

In some embodiments, L is

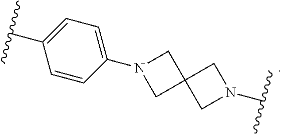 [SMILES: *c1ccc(N2CC3(CN(*)C3)C2)cc1]

In some embodiments, L is

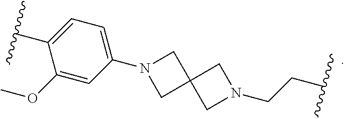 [SMILES: *CCN1CC2(C1)CN(c1ccc(*)c(OC)c1)C2]

In some embodiments, L is

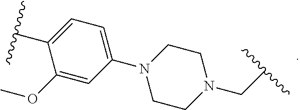 [SMILES: *CN1CCN(c2ccc(*)c(OC)c2)CC1]

In some embodiments, L is

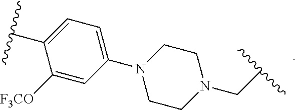 [SMILES: *CN1CCN(c2ccc(*)c(OC(F)(F)F)c2)CC1]

In some embodiments, L is

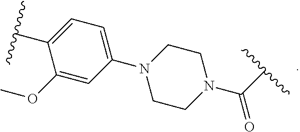 [SMILES: *C(=O)N1CCN(c2ccc(*)c(OC)c2)CC1]

In some embodiments, L is

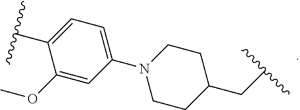 [SMILES: *CC1CCN(c2ccc(*)c(OC)c2)CC1]

In some embodiments, L is

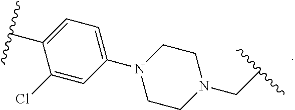 [SMILES: *CN1CCN(c2ccc(*)c(Cl)c2)CC1]

In some embodiments, L is

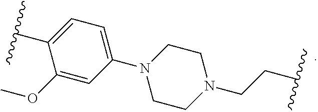 [SMILES: *CCN1CCN(c2ccc(*)c(OC)c2)CC1]

In some embodiments, L is

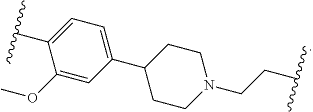 [SMILES: *CCN1CCC(c2ccc(*)c(OC)c2)CC1]

In some embodiments, L is

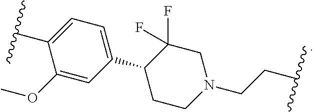 [SMILES: *CCN1CC[C@H](c2ccc(*)c(OC)c2)C(F)(F)C1]

In some embodiments, L is

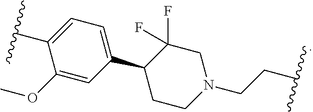 [SMILES: *CCN1CC[C@@H](c2ccc(*)c(OC)c2)C(F)(F)C1]

In some embodiments, L is

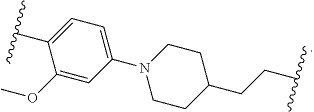 [SMILES: *CCC1CCN(c2ccc(*)c(OC)c2)CC1]

In some embodiments, L is

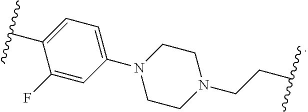 [SMILES: *CCN1CCN(c2ccc(*)c(F)c2)CC1]

In some embodiments, L is

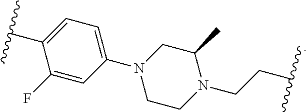 [SMILES: *CCN1CCN(c2ccc(*)c(F)c2)C[C@H]1C]

In some embodiments, L is

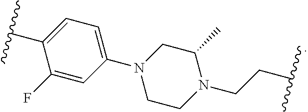 [SMILES: *CCN1CCN(c2ccc(*)c(F)c2)C[C@@H]1C]

In some embodiments, L is

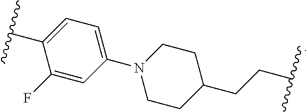 [SMILES: *CCC1CCN(c2ccc(*)c(F)c2)CC1]

In some embodiments, L is

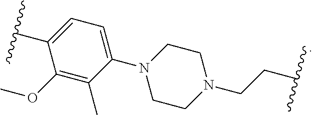 [SMILES: *CCN1CCN(c2ccc(*)c(OC)c2C)CC1]

In some embodiments, L is

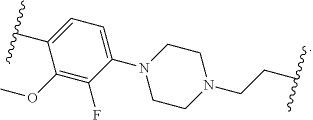 [SMILES: *CCN1CCN(c2ccc(*)c(OC)c2F)CC1]

In some embodiments, L is

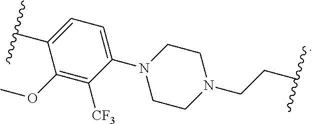 [SMILES: *CCN1CCN(c2ccc(*)c(OC)c2C(F)(F)F)CC1]

In some embodiments, L is

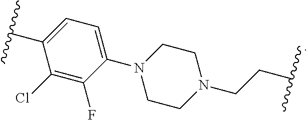 [SMILES: *CCN1CCN(c2ccc(*)c(Cl)c2F)CC1]

In some embodiments, L is

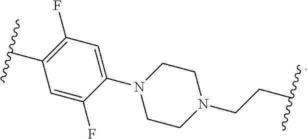 [SMILES: *CCN1CCN(c2cc(F)c(*)cc2F)CC1]

In some embodiments, L is

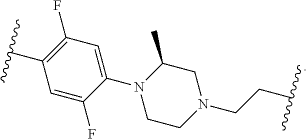 [SMILES: *CCN1CCN(c2cc(F)c(*)cc2F)[C@@H](C)C1]

In some embodiments, L is

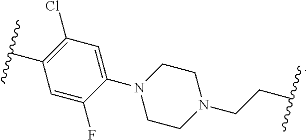 [SMILES: *CCN1CCN(c2cc(Cl)c(*)cc2F)CC1]

In some embodiments, L is

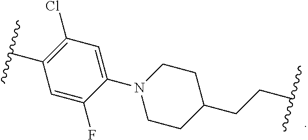 [SMILES: *CCC1CCN(c2cc(Cl)c(*)cc2F)CC1]

In some embodiments, L is

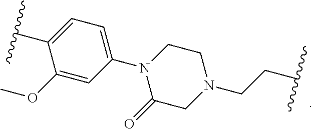 [SMILES: *CCN1CCN(c2ccc(*)c(OC)c2)C(=O)C1]

In some embodiments, L is

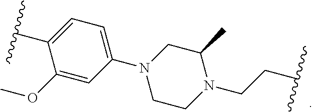 [SMILES: *CCN1CCN(c2ccc(*)c(OC)c2)C[C@H]1C]

In some embodiments, L is

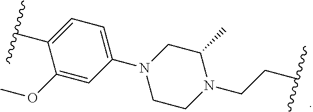 [SMILES: *CCN1CCN(c2ccc(*)c(OC)c2)C[C@@H]1C]

In some embodiments, L is

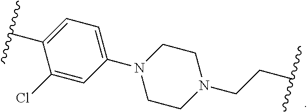 [SMILES: *CCN1CCN(c2ccc(*)c(Cl)c2)CC1]

In some embodiments, L is

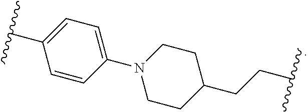 [SMILES: *CCC1CCN(c2ccc(*)cc2)CC1]

In some embodiments, L is

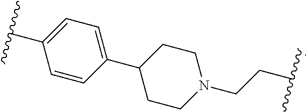 [SMILES: *CCN1CCC(c2ccc(*)cc2)CC1]

In some embodiments, L is

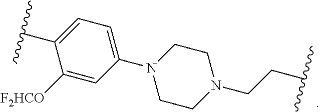 [SMILES: *CCN1CCN(c2ccc(*)c(OC(F)F)c2)CC1]

In some embodiments, L is

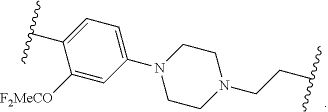

In some embodiments, L is

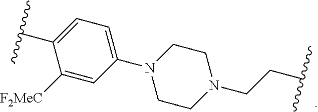

In some embodiments, L is

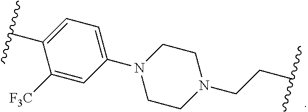 [SMILES: *CCN1CCN(c2ccc(*)c(C(F)(F)F)c2)CC1]

In some embodiments, L is

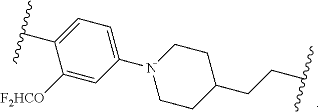 [SMILES: *CCC1CCN(c2ccc(*)c(OC(F)F)c2)CC1]

In some embodiments, L is

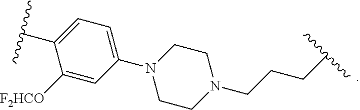 [SMILES: *CCCN1CCN(c2ccc(*)c(OC(F)F)c2)CC1]

In some embodiments, L is

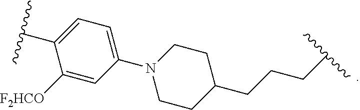 [SMILES: *CCCC1CCN(c2ccc(*)c(OC(F)F)c2)CC1]

In some embodiments, L is

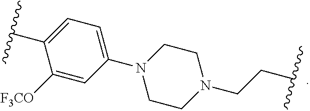 [SMILES: *CCN1CCN(c2ccc(*)c(OC(F)(F)F)c2)CC1]

In some embodiments, L is

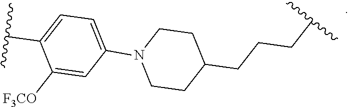 [SMILES: *CCCC1CCN(c2ccc(*)c(OC(F)(F)F)c2)CC1]

In some embodiments, L is

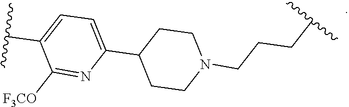 [SMILES: *CCCN1CCC(c2ccc(*)c(OC(F)(F)F)n2)CC1]

In some embodiments, L is

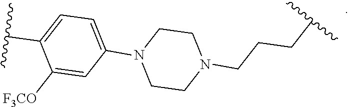 [SMILES: *CCCN1CCN(c2ccc(*)c(OC(F)(F)F)c2)CC1]

In some embodiments, L is

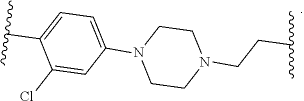 [SMILES: *CCN1CCN(c2ccc(*)c(Cl)c2)CC1]

In some embodiments, L is

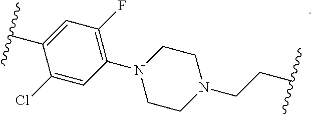 [SMILES: *CCN1CCN(c2cc(Cl)c(*)cc2F)CC1]

In some embodiments, L is

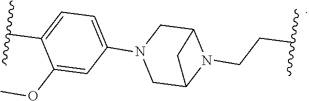 [SMILES: *CCN1C2CC1CN(c1ccc(*)c(OC)c1)C2]

In some embodiments, L is

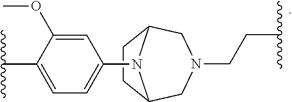 [SMILES: *CCN1CC2CCC(C1)N2c1ccc(*)c(OC)c1]

In some embodiments, L is

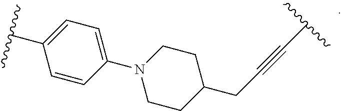 [SMILES: *C#CCC1CCN(c2ccc(*)cc2)CC1]

In some embodiments, L is

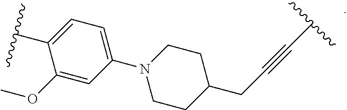 [SMILES: *C#CCC1CCN(c2ccc(*)c(OC)c2)CC1]

In some embodiments, L is

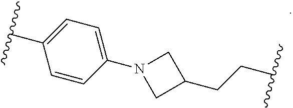 [SMILES: *CCC1CN(c2ccc(*)cc2)C1]

In some embodiments, L is

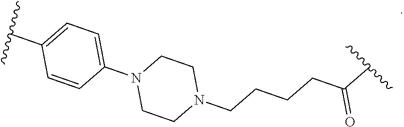 [SMILES: *C(=O)CCCCN1CCN(c2ccc(*)cc2)CC1]

In some embodiments, L is

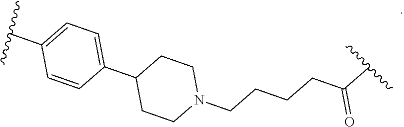 [SMILES: *C(=O)CCCCN1CCC(c2ccc(*)cc2)CC1]

In some embodiments, L is

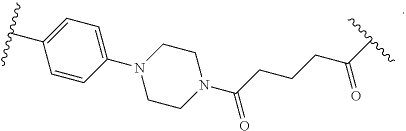 [SMILES: *C(=O)CCCC(=O)N1CCN(c2ccc(*)cc2)CC1]

In some embodiments, L is

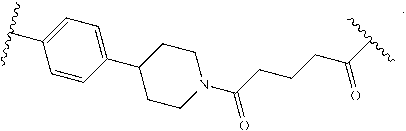 [SMILES: *C(=O)CCCC(=O)N1CCC(c2ccc(*)cc2)CC1]

In some embodiments, L is

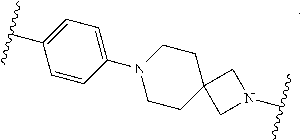 [SMILES: *c1ccc(N2CCC3(CC2)CN(*)C3)cc1]

In some embodiments, L is

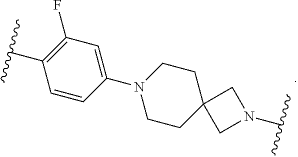 [SMILES: *c1ccc(N2CCC3(CC2)CN(*)C3)cc1F]

In some embodiments, L is
In some embodiments, L is

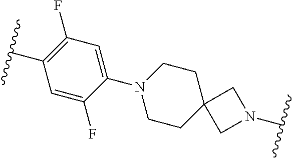 [SMILES: *c1cc(F)c(N2CCC3(CC2)CN(*)C3)cc1F]

In some embodiments, L is

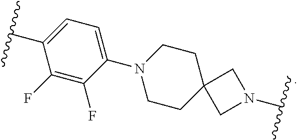 [SMILES: *c1ccc(N2CCC3(CC2)CN(*)C3)c(F)c1F]

In some embodiments, L is

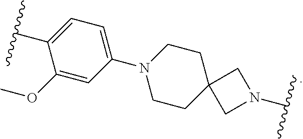 [SMILES: *c1ccc(N2CCC3(CC2)CN(*)C3)cc1OC]

In some embodiments, L is

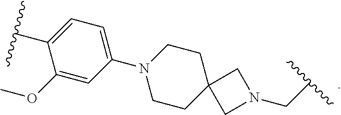 [SMILES: *CN1CC2(CCN(c3ccc(*)c(OC)c3)CC2)C1]

In some embodiments, L is

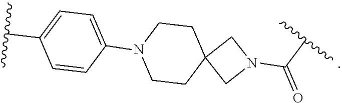 [SMILES: *C(=O)N1CC2(CCN(c3ccc(*)cc3)CC2)C1]

In some embodiments, L is

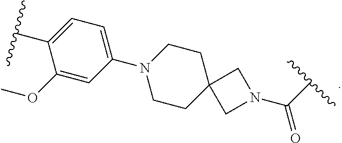 [SMILES: *C(=O)N1CC2(CCN(c3ccc(*)c(OC)c3)CC2)C1]

In some embodiments, L is

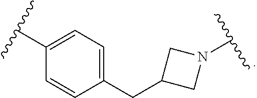 [SMILES: *c1ccc(CC2CN(*)C2)cc1]

In some embodiments, L is

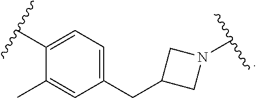 [SMILES: *c1ccc(CC2CN(*)C2)cc1C]

In some embodiments, L is

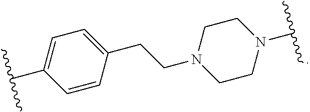 [SMILES: *c1ccc(CCN2CCN(*)CC2)cc1]

In some embodiments, L is

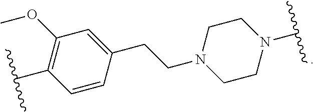 [SMILES: *c1ccc(CCN2CCN(*)CC2)cc1OC]

In some embodiments, L is

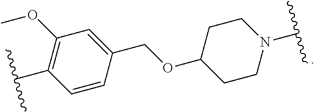 [SMILES: *c1ccc(COC2CCN(*)CC2)cc1OC]

In some embodiments, L is

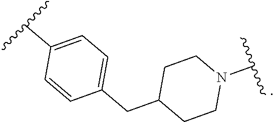 [SMILES: *c1ccc(CC2CCN(*)CC2)cc1]

In some embodiments, L is

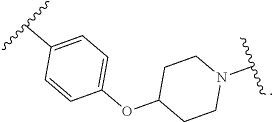 [SMILES: *c1ccc(OC2CCN(*)CC2)cc1]

In some embodiments, L is

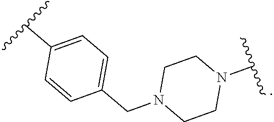 [SMILES: *c1ccc(CN2CCN(*)CC2)cc1]

In some embodiments, L is

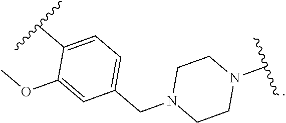 [SMILES: *c1ccc(CN2CCN(*)CC2)cc1OC]

In some embodiments, L is

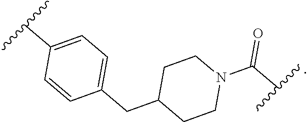 [SMILES: *C(=O)N1CCC(Cc2ccc(*)cc2)CC1]

In some embodiments, L is

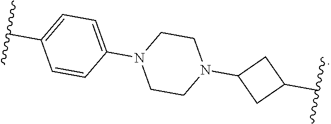 [SMILES: *c1ccc(N2CCN(C3CC(*)C3)CC2)cc1]

In some embodiments, L is

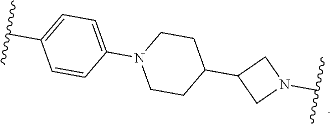 [SMILES: *c1ccc(N2CCC(C3CN(*)C3)CC2)cc1]

In some embodiments, L is

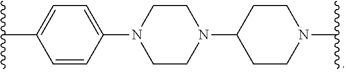 [SMILES: *c1ccc(N2CCN(C3CCN(*)CC3)CC2)cc1]

In some embodiments, L is

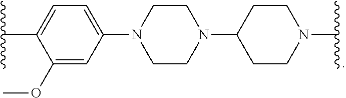 [SMILES: *c1ccc(N2CCN(C3CCN(*)CC3)CC2)cc1OC]

In some embodiments, L is

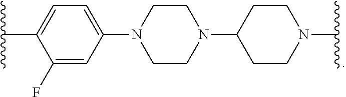 [SMILES: *c1ccc(N2CCN(C3CCN(*)CC3)CC2)cc1F]

In some embodiments, L is

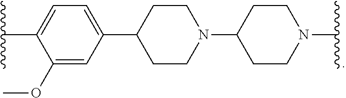 [SMILES: *c1ccc(C2CCN(C3CCN(*)CC3)CC2)cc1OC]

In some embodiments, L is

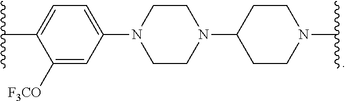 [SMILES: *c1ccc(N2CCN(C3CCN(*)CC3)CC2)cc1OC(F)(F)F]

In some embodiments, L is

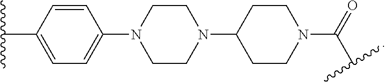 [SMILES: *C(=O)N1CCC(N2CCN(c3ccc(*)cc3)CC2)CC1]

In some embodiments, L is

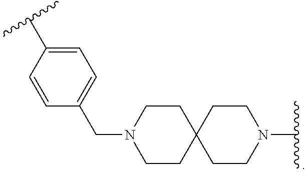 [SMILES: *c1ccc(CN2CCC3(CCN(*)CC3)CC2)cc1]

In some embodiments, L is

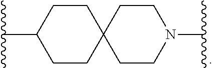 [SMILES: *C1CCC2(CC1)CCN(*)CC2]

In some embodiments, L is

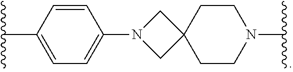 [SMILES: *c1ccc(N2CC3(CCN(*)CC3)C2)cc1]

In some embodiments, L is

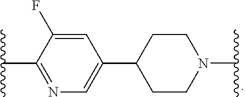 [SMILES: *c1ncc(C2CCN(*)CC2)cc1F]

In some embodiments, L is

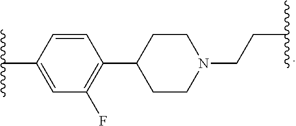 [SMILES: *CCN1CCC(c2ccc(*)cc2F)CC1]

In some embodiments, L is

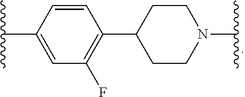 [SMILES: *c1ccc(C2CCN(*)CC2)c(F)c1]

In some embodiments, L is

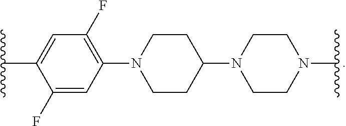 [SMILES: *c1cc(F)c(N2CCC(N3CCN(*)CC3)CC2)cc1F]

In some embodiments, L is

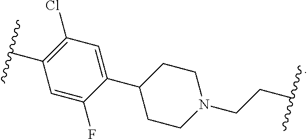 [SMILES: *CCN1CCC(c2cc(Cl)c(*)cc2F)CC1]

In some embodiments, L is

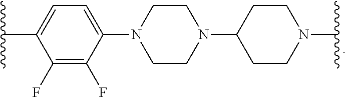 [SMILES: *c1ccc(N2CCN(C3CCN(*)CC3)CC2)c(F)c1F]

In some embodiments, L is

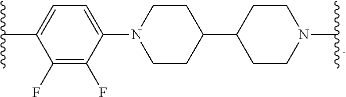 [SMILES: *c1ccc(N2CCC(C3CCN(*)CC3)CC2)c(F)c1F]

In some embodiments, L is

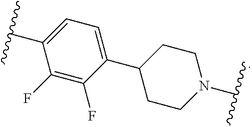 [SMILES: *c1ccc(C2CCN(*)CC2)c(F)c1F]

In some embodiments, L is

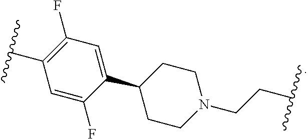

In some embodiments, L is

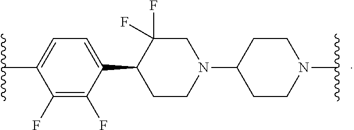 [SMILES: *c1ccc([C@@H]2CCN(C3CCN(*)CC3)CC2(F)F)c(F)c1F]

In some embodiments, L is

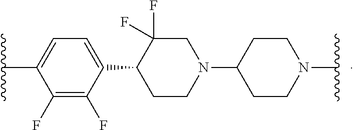 [SMILES: *c1ccc([C@H]2CCN(C3CCN(*)CC3)CC2(F)F)c(F)c1F]

In some embodiments, L is

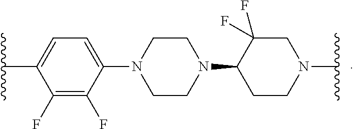 [SMILES: *c1ccc(N2CCN([C@@H]3CCN(*)CC3(F)F)CC2)c(F)c1F]

In some embodiments, L is

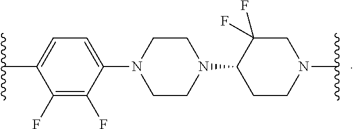 [SMILES: *c1ccc(N2CCN([C@H]3CCN(*)CC3(F)F)CC2)c(F)c1F]

In some embodiments, L is

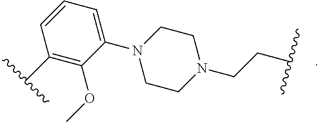 [SMILES: *CCN1CCN(c2cccc(*)c2OC)CC1]

In some embodiments, L is

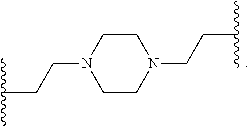 [SMILES: *CCN1CCN(CC*)CC1]

In some embodiments, L is

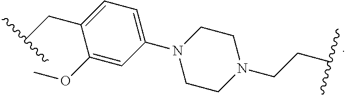 [SMILES: *CCN1CCN(c2ccc(C*)c(OC)c2)CC1]

In some embodiments, L is

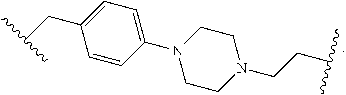 [SMILES: *CCN1CCN(c2ccc(C*)cc2)CC1]

In some embodiments, L is

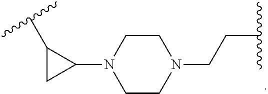 [SMILES: *CCN1CCN(C2CC2*)CC1]

In some embodiments, L is

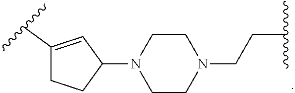 [SMILES: *CCN1CCN(C2C=C(*)CC2)CC1]

In some embodiments, L is

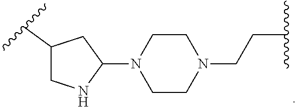 [SMILES: *CCN1CCN(C2CC(*)CN2)CC1]

In some embodiments, L is

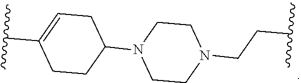 [SMILES: *CCN1CCN(C2CC=C(*)CC2)CC1]

In some embodiments, L is

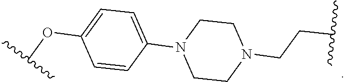 [SMILES: *CCN1CCN(c2ccc(O*)cc2)CC1]

In some embodiments, L is

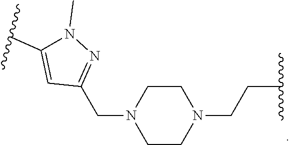 [SMILES: *CCN1CCN(Cc2cc(*)n(C)n2)CC1]

In some embodiments, L is

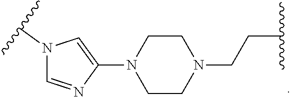 [SMILES: *CCN1CCN(c2cn(*)cn2)CC1]

In some embodiments, L is

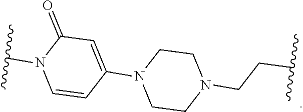 [SMILES: *CCN1CCN(c2ccn(*)c(=O)c2)CC1]

In some embodiments, L is

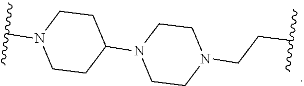 [SMILES: *CCN1CCN(C2CCN(*)CC2)CC1]

In some embodiments, L is

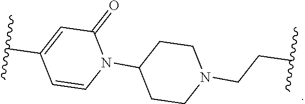 [SMILES: *CCN1CCC(n2ccc(*)cc2=O)CC1]

In some embodiments, L is

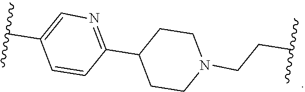 [SMILES: *CCN1CCC(c2ccc(*)cn2)CC1]

In some embodiments, L is

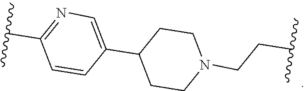 [SMILES: *CCN1CCC(c2ccc(*)nc2)CC1]

In some embodiments, L is

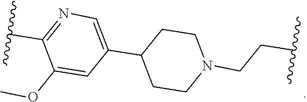 [SMILES: *CCN1CCC(c2cnc(*)c(OC)c2)CC1]

In some embodiments, L is

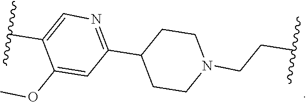 [SMILES: *CCN1CCC(c2cc(OC)c(*)cn2)CC1]

In some embodiments, L is

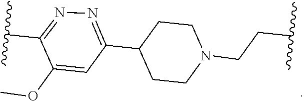 [SMILES: *CCN1CCC(c2cc(OC)c(*)nn2)CC1]

In some embodiments, L is

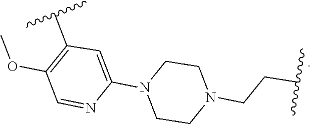 [SMILES: *CCN1CCN(c2cc(*)c(OC)cn2)CC1]

In some embodiments, L is

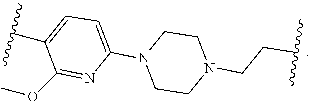 [SMILES: *CCN1CCN(c2ccc(*)c(OC)n2)CC1]

In some embodiments, L is

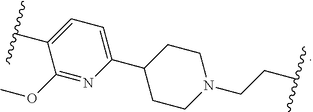 [SMILES: *CCN1CCC(c2ccc(*)c(OC)n2)CC1]

In some embodiments, L is

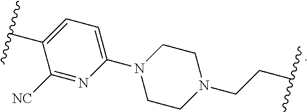 [SMILES: *CCN1CCN(c2ccc(*)c(C#N)n2)CC1]

In some embodiments, L is

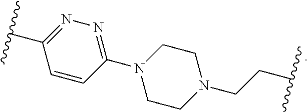 [SMILES: *CCN1CCN(c2ccc(*)nn2)CC1]

In some embodiments, L is

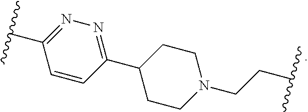 [SMILES: *CCN1CCC(c2ccc(*)nn2)CC1]

In some embodiments, L is

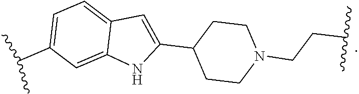 [SMILES: *CCN1CCC(c2cc3ccc(*)cc3[nH]2)CC1]

In some embodiments, L is

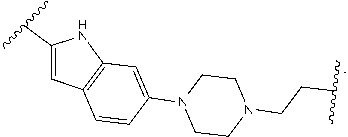 [SMILES: *CCN1CCN(c2ccc3cc(*)[nH]c3c2)CC1]

In some embodiments, L is

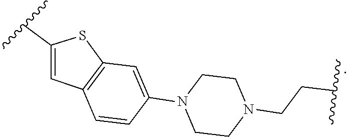 [SMILES: *CCN1CCN(c2ccc3cc(*)sc3c2)CC1]

In some embodiments, L is

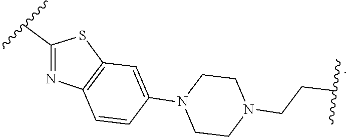 [SMILES: *CCN1CCN(c2ccc3nc(*)sc3c2)CC1]

In some embodiments, L is

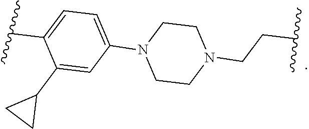 [SMILES: *CCN1CCN(c2ccc(*)c(C3CC3)c2)CC1]

In some embodiments, L is

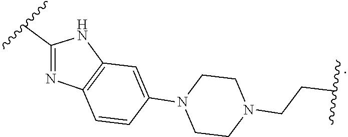 [SMILES: *CCN1CCN(c2ccc3nc(*)[nH]c3c2)CC1]

In some embodiments, L is

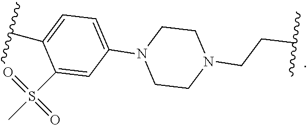 [SMILES: *CCN1CCN(c2ccc(*)c(S(C)(=O)=O)c2)CC1]

In some embodiments, L is

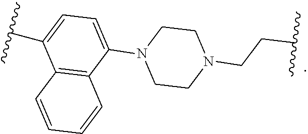 [SMILES: *CCN1CCN(c2ccc(*)c3ccccc23)CC1]

In some embodiments, L is

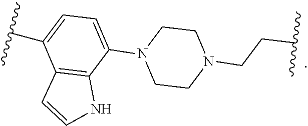 [SMILES: *CCN1CCN(c2ccc(*)c3cc[nH]c23)CC1]

In some embodiments, L is

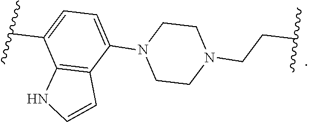 [SMILES: *CCN1CCN(c2ccc(*)c3[nH]ccc23)CC1]

In some embodiments, L is

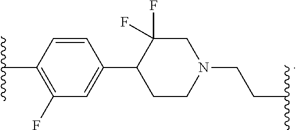 [SMILES: *CCN1CCC(c2ccc(*)c(F)c2)C(F)(F)C1]

In some embodiments, L is

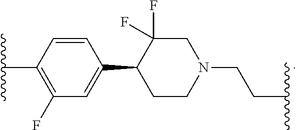 [SMILES: *CCN1CC[C@@H](c2ccc(*)c(F)c2)C(F)(F)C1]

In some embodiments, L is

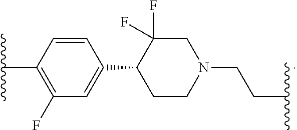 [SMILES: *CCN1CC[C@H](c2ccc(*)c(F)c2)C(F)(F)C1]

In some embodiments, L is

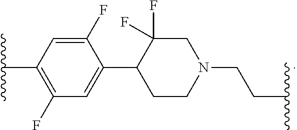 [SMILES: *CCN1CCC(c2cc(F)c(*)cc2F)C(F)(F)C1]

In some embodiments, L is

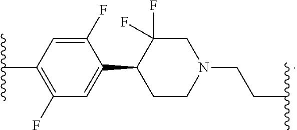 [SMILES: *CCN1CC[C@@H](c2cc(F)c(*)cc2F)C(F)(F)C1]

In some embodiments, L is

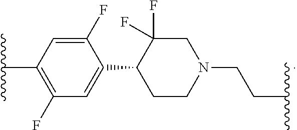 [SMILES: *CCN1CC[C@H](c2cc(F)c(*)cc2F)C(F)(F)C1]

In some embodiments, L is

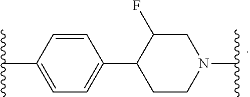 [SMILES: *c1ccc(C2CCN(*)CC2F)cc1]

In some embodiments, L is

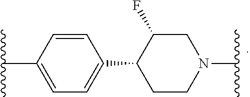 [SMILES: *c1ccc([C@H]2CCN(*)C[C@H]2F)cc1]

In some embodiments, L is

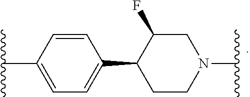 [SMILES: *c1ccc([C@@H]2CCN(*)C[C@@H]2F)cc1]

In some embodiments, L is

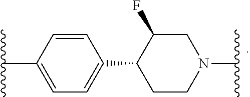 [SMILES: *c1ccc([C@H]2CCN(*)C[C@@H]2F)cc1]

In some embodiments, L is

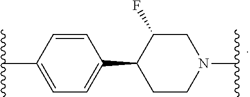 [SMILES: *c1ccc([C@@H]2CCN(*)C[C@H]2F)cc1]

In some embodiments, L is

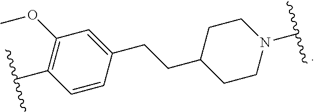 [SMILES: *c1ccc(CCC2CCN(*)CC2)cc1OC]

In some embodiments, L is

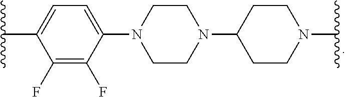 [SMILES: *c1ccc(N2CCN(C3CCN(*)CC3)CC2)c(F)c1F]

In some embodiments, L is

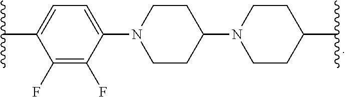 [SMILES: *c1ccc(N2CCC(N3CCC(*)CC3)CC2)c(F)c1F]

In some embodiments, L is selected from those depicted in Table 1A, below. In some embodiments, LBM is selected from those depicted in Table A, below. In some embodiments, L is selected from those depicted in Table B, below.

TABLE A
Exemplified E3 Ligase Binding Moiety (LBM)

| | |
|---|---|
| 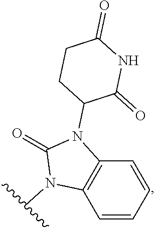 [SMILES: *n1c(=O)n(C2CCC(=O)NC2=O)c2ccccc21] | (e) |
| 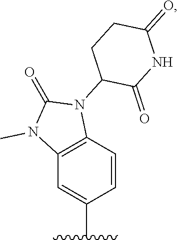 [SMILES: *c1ccc2c(c1)n(C)c(=O)n2C1CCC(=O)NC1=O] | (f) |
| 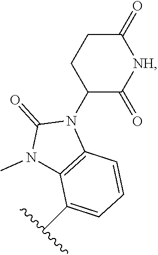 [SMILES: *c1cccc2c1n(C)c(=O)n2C1CCC(=O)NC1=O] | (g) |
| 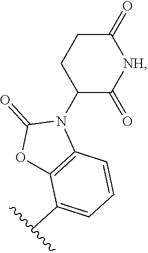 [SMILES: *c1cccc2c1oc(=O)n2C1CCC(=O)NC1=O] | (h) |
| 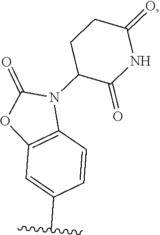 [SMILES: *c1ccc2c(c1)oc(=O)n2C1CCC(=O)NC1=O] | (i) |
| 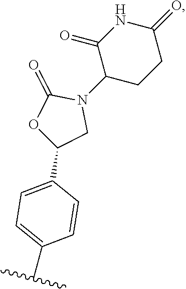 [SMILES: *c1ccc([C@H]2CN(C3CCC(=O)NC3=O)C(=O)O2)cc1] | (j) |
| 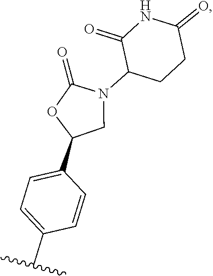 [SMILES: *c1ccc([C@@H]2CN(C3CCC(=O)NC3=O)C(=O)O2)cc1] | (k) |
| 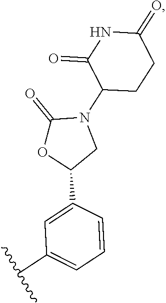 [SMILES: *c1cccc([C@H]2CN(C3CCC(=O)NC3=O)C(=O)O2)c1] | (l) |
| 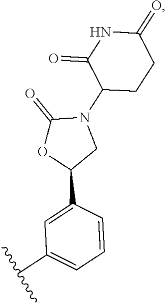 [SMILES: *c1cccc([C@@H]2CN(C3CCC(=O)NC3=O)C(=O)O2)c1] | (m) |
| 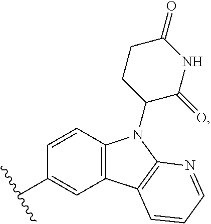 [SMILES: *c1ccc2c(c1)c1cccnc1n2C1CCC(=O)NC1=O] | (n) |
| 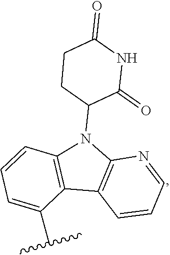 [SMILES: *c1cccc2c1c1cccnc1n2C1CCC(=O)NC1=O] | (o) |
| 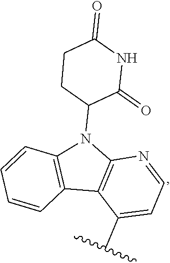 [SMILES: *c1ccnc2c1c1ccccc1n2C1CCC(=O)NC1=O] | (p) |
| 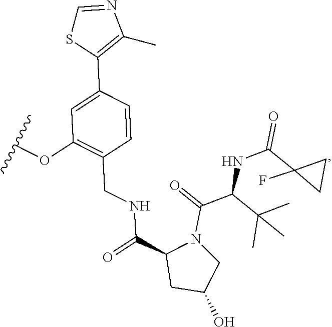 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (n) |
| 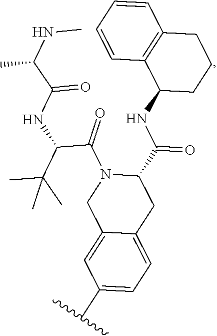 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (o) |
| 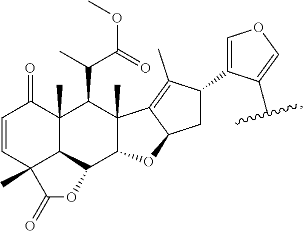 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (p) |
| 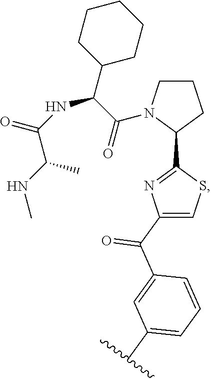 [SMILES: *c1cccc(C(=O)c2csc([C@@H]3CCCN3C(=O)[C@@H](NC(=O)[C@H](C)NC)C3CCCCC3)n2)c1] | (q) |
| 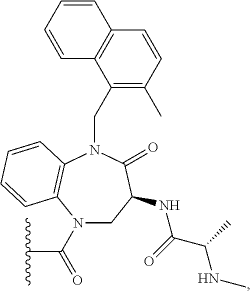 [SMILES: *C(=O)N1C[C@H](NC(=O)[C@H](C)NC)C(=O)N(Cc2c(C)ccc3ccccc23)c2ccccc21] | (r) |
| 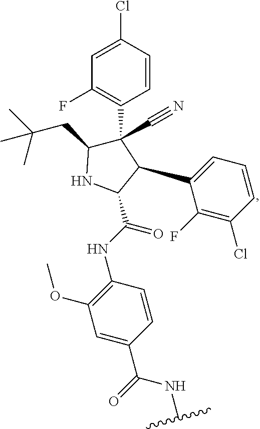 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (s) |
| 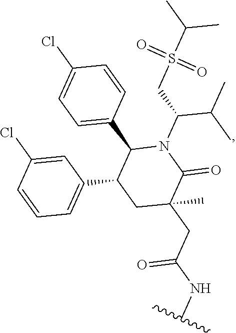 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (t) |
| 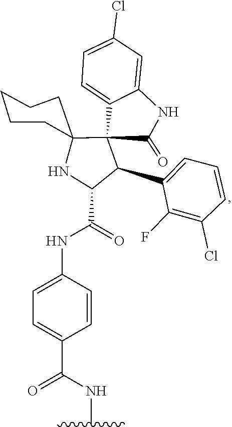 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (u) |
| 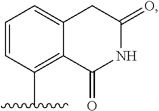 [SMILES: *c1cccc2c1C(=O)NC(=O)C2] | (v) |
| 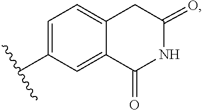 [SMILES: *c1ccc2c(c1)C(=O)NC(=O)C2] | (w) |
| 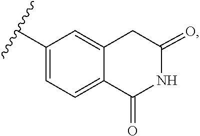 [SMILES: *c1ccc2c(c1)CC(=O)NC2=O] | (x) |
| 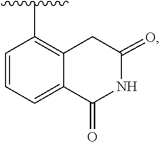 [SMILES: *c1cccc2c1CC(=O)NC2=O] | (y) |
| 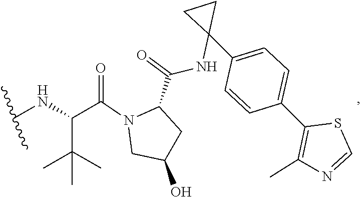 [SMILES: *N[C@H](C(=O)N1C[C@H](O)C[C@H]1C(=O)NC1(c2ccc(-c3scnc3C)cc2)CC1)C(C)(C)C] | (z) |
| 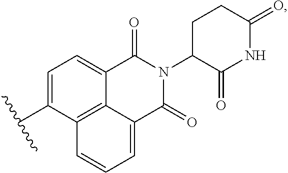 [SMILES: *c1ccc2c3c(cccc13)C(=O)N(C1CCC(=O)NC1=O)C2=O] | (bb) |
| 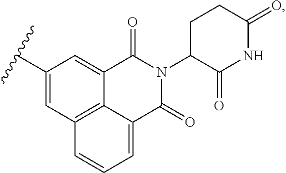 [SMILES: *c1cc2c3c(cccc3c1)C(=O)N(C1CCC(=O)NC1=O)C2=O] | (cc) |
| 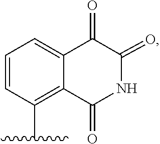 [SMILES: *c1cccc2c1C(=O)NC(=O)C2=O] | (dd) |
| 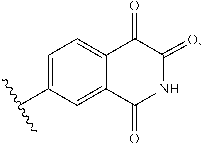 [SMILES: *c1ccc2c(c1)C(=O)NC(=O)C2=O] | (ee) |
| 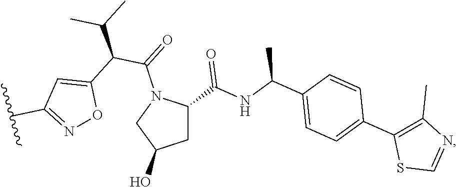 [SMILES: *c1cc([C@H](C(=O)N2C[C@H](O)C[C@H]2C(=O)N[C@@H](C)c2ccc(-c3scnc3C)cc2)C(C)C)on1] | (ff) |
| 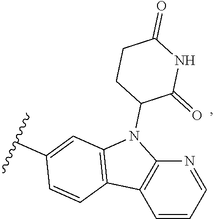 [SMILES: *c1ccc2c3cccnc3n(C3CCC(=O)NC3=O)c2c1] | (gg) |
| 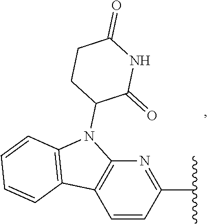 [SMILES: *c1ccc2c3ccccc3n(C3CCC(=O)NC3=O)c2n1] | (hh) |
| 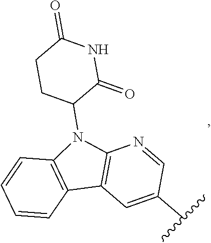 [SMILES: *c1cnc2c(c1)c1ccccc1n2C1CCC(=O)NC1=O] | (ii) |
| 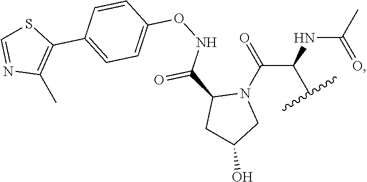 [SMILES: *[C@H](NC(C)=O)C(=O)N1C[C@H](O)C[C@H]1C(=O)NOc1ccc(-c2scnc2C)cc1] | (jj) |
| 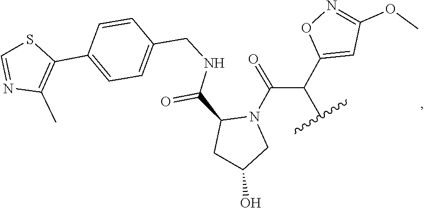 [SMILES: *C(C(=O)N1C[C@H](O)C[C@H]1C(=O)NCc1ccc(-c2scnc2C)cc1)c1cc(OC)no1] | (kk) |
| 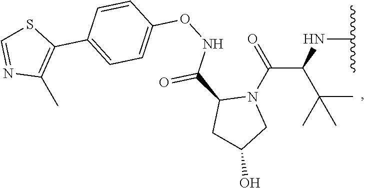 [SMILES: *N[C@H](C(=O)N1C[C@H](O)C[C@H]1C(=O)NOc1ccc(-c2scnc2C)cc1)C(C)(C)C] | (ll) |
| 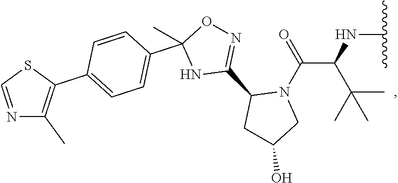 [SMILES: *N[C@H](C(=O)N1C[C@H](O)C[C@H]1C1=NOC(C)(c2ccc(-c3scnc3C)cc2)N1)C(C)(C)C] | (mm) |
| 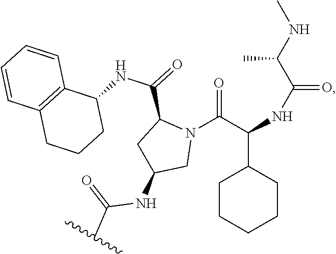 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (nn) |
| 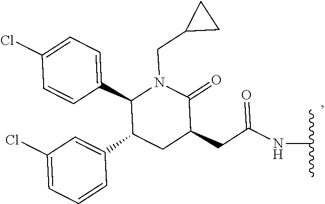 [SMILES: *NC(=O)C[C@H]1C[C@H](c2cccc(Cl)c2)[C@@H](c2ccc(Cl)cc2)N(CC2CC2)C1=O] | (oo) |
| 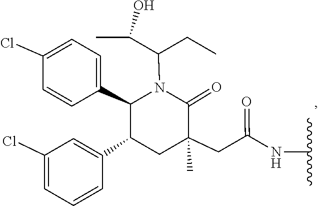 [SMILES: *NC(=O)C[C@@]1(C)C[C@H](c2cccc(Cl)c2)[C@@H](c2ccc(Cl)cc2)N(C(CC)[C@H](C)O)C1=O] | (pp) |
| 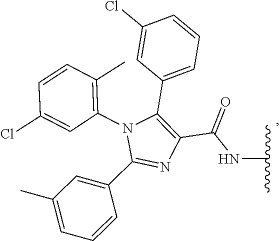 [SMILES: *NC(=O)c1nc(-c2cccc(C)c2)n(-c2cc(Cl)ccc2C)c1-c1cccc(Cl)c1] | (qq) |
| 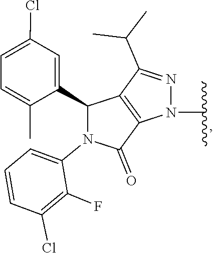 [SMILES: *n1nc(C(C)C)c2c1C(=O)N(c1cccc(Cl)c1F)[C@H]2c1cc(Cl)ccc1C] | (rr) |
| 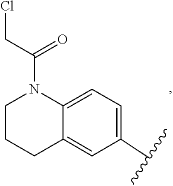 [SMILES: *c1ccc2c(c1)CCCN2C(=O)CCl ; ","] | (ss) |
| 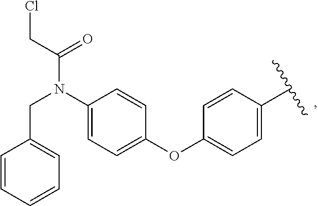 [SMILES: *c1ccc(Oc2ccc(N(Cc3ccccc3)C(=O)CCl)cc2)cc1] | (tt) |
| 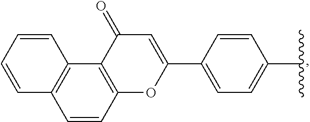 [SMILES: *c1ccc(-c2cc(=O)c3c(ccc4ccccc43)o2)cc1] | (uu) |
| 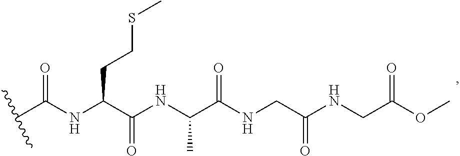 [SMILES: *C(=O)N[C@@H](CCSC)C(=O)N[C@@H](C)C(=O)NCC(=O)NCC(=O)OC] | (vv) |
| 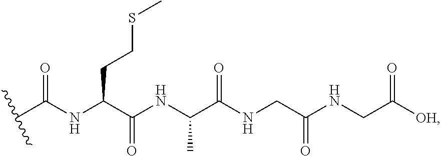 [SMILES: *C(=O)N[C@@H](CCSC)C(=O)N[C@@H](C)C(=O)NCC(=O)NCC(=O)O] | (ww) |
| 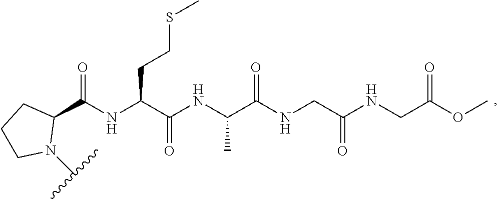 [SMILES: *N1CCC[C@H]1C(=O)N[C@@H](CCSC)C(=O)N[C@@H](C)C(=O)NCC(=O)NCC(=O)OC] | (xx) |
| 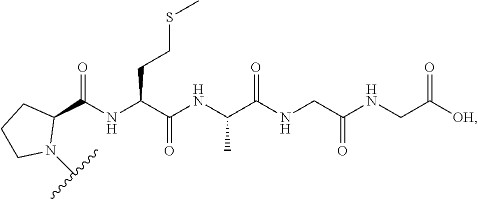 [SMILES: *N1CCC[C@H]1C(=O)N[C@@H](CCSC)C(=O)N[C@@H](C)C(=O)NCC(=O)NCC(=O)O] | (yy) |
| 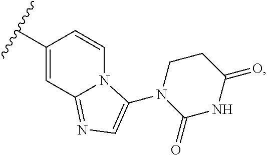 [SMILES: *c1ccn2c(N3CCC(=O)NC3=O)cnc2c1] | (zz) |
| 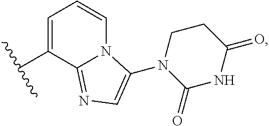 [SMILES: *c1cccn2c(N3CCC(=O)NC3=O)cnc12] | (aaa) |
| 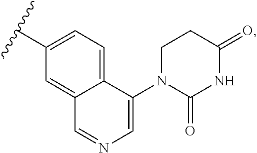 [SMILES: *c1ccc2c(N3CCC(=O)NC3=O)cncc2c1] | (bbb) |
| 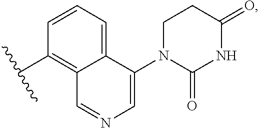 [SMILES: *c1cccc2c(N3CCC(=O)NC3=O)cncc12] | (ccc) |
| 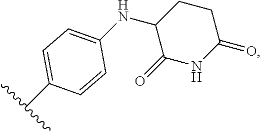 [SMILES: *c1ccc(NC2CCC(=O)NC2=O)cc1] | (ddd) |
| 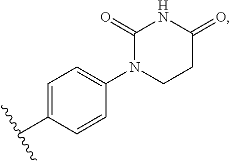 [SMILES: *c1ccc(N2CCC(=O)NC2=O)cc1] | (eee) |
| 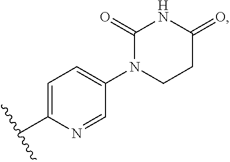 [SMILES: *c1ccc(N2CCC(=O)NC2=O)cn1] | (fff) |
| 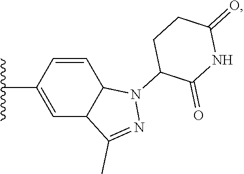 [SMILES: *C1=CC2C(C)=NN(C3CCC(=O)NC3=O)C2C=C1] | (ggg) |
| 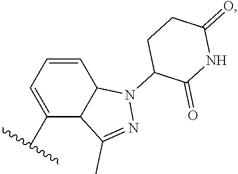 [SMILES: *C1=CC=CC2C1C(C)=NN2C1CCC(=O)NC1=O] | (hhh) |
| 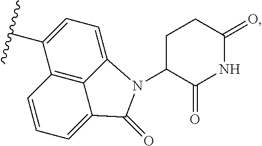 [SMILES: *c1ccc2c3c(cccc13)C(=O)N2C1CCC(=O)NC1=O] | (iii) |
| 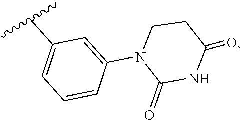 [SMILES: *c1cccc(N2CCC(=O)NC2=O)c1] | (jjj) |
| 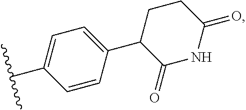 [SMILES: *c1ccc(C2CCC(=O)NC2=O)cc1] | (kkk) |
| 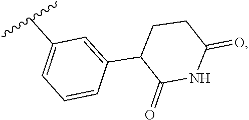 [SMILES: *c1cccc(C2CCC(=O)NC2=O)c1] | (lll) |
| 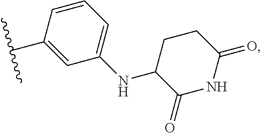 [SMILES: *c1cccc(NC2CCC(=O)NC2=O)c1] | (mmm) |
| 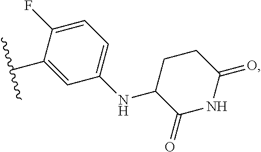 [SMILES: *c1cc(NC2CCC(=O)NC2=O)ccc1F] | (nnn) |
| 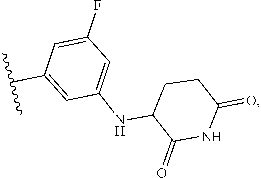 [SMILES: *c1cc(F)cc(NC2CCC(=O)NC2=O)c1] | (ooo) |
| 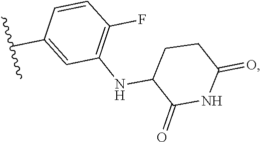 [SMILES: *c1ccc(F)c(NC2CCC(=O)NC2=O)c1] | (ppp) |
| 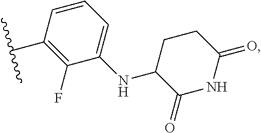 [SMILES: *c1cccc(NC2CCC(=O)NC2=O)c1F] | (qqq) |
| 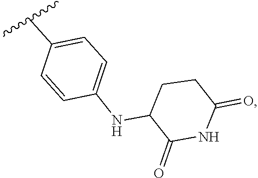 [SMILES: *c1ccc(NC2CCC(=O)NC2=O)cc1] | (rrr) |
| 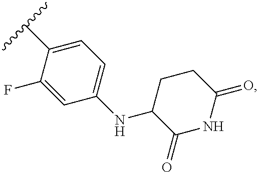 [SMILES: *c1ccc(NC2CCC(=O)NC2=O)cc1F] | (sss) |
| 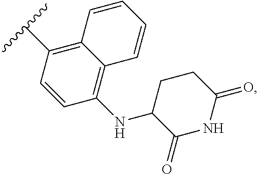 [SMILES: *c1ccc(NC2CCC(=O)NC2=O)c2ccccc12] | (ttt) |
| 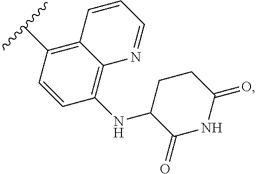 [SMILES: *c1ccc(NC2CCC(=O)NC2=O)c2ncccc12] | (uuu) |
| 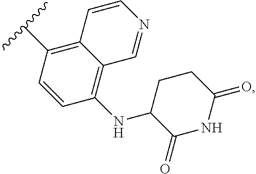 [SMILES: *c1ccc(NC2CCC(=O)NC2=O)c2cnccc12] | (vvv) |
| 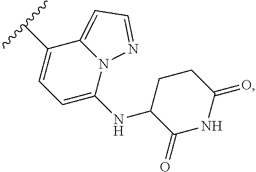 [SMILES: *c1ccc(NC2CCC(=O)NC2=O)n2nccc12] | (www) |
| 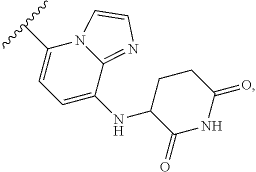 [SMILES: *c1ccc(NC2CCC(=O)NC2=O)c2nccn12] | (xxx) |
| 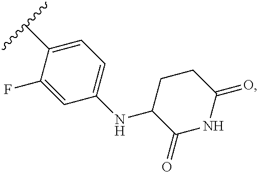 [SMILES: *c1ccc(NC2CCC(=O)NC2=O)cc1F] | (yyy) |
| 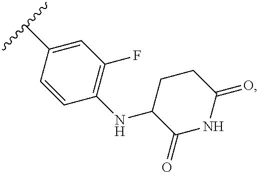 [SMILES: *c1ccc(NC2CCC(=O)NC2=O)c(F)c1] | (zzz) |
| 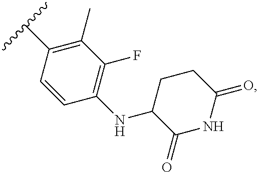 [SMILES: *c1ccc(NC2CCC(=O)NC2=O)c(F)c1C] | (aaaa) |
| 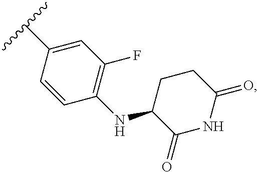 [SMILES: *c1ccc(N[C@H]2CCC(=O)NC2=O)c(F)c1] | (bbbb) |
| 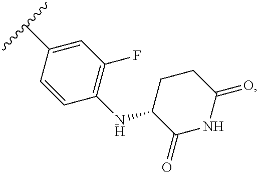 [SMILES: *c1ccc(N[C@@H]2CCC(=O)NC2=O)c(F)c1] | (cccc) |
| 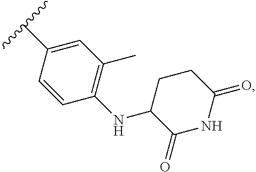 [SMILES: *c1ccc(NC2CCC(=O)NC2=O)c(C)c1] | (dddd) |
| 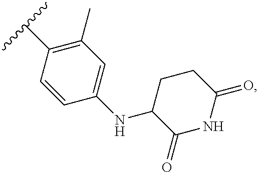 [SMILES: *c1ccc(NC2CCC(=O)NC2=O)cc1C] | (eeee) |
| 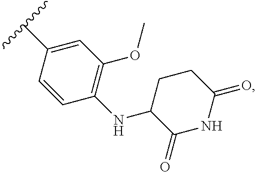 [SMILES: *c1ccc(NC2CCC(=O)NC2=O)c(OC)c1] | (ffff) |
| 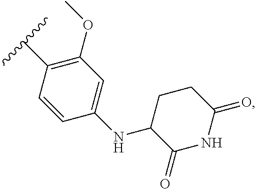 [SMILES: *c1ccc(NC2CCC(=O)NC2=O)cc1OC] | (gggg) |
| 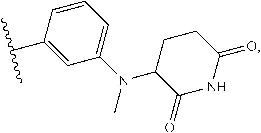 [SMILES: *c1cccc(N(C)C2CCC(=O)NC2=O)c1] | (hhhh) |
| 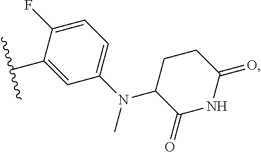 [SMILES: *c1cc(N(C)C2CCC(=O)NC2=O)ccc1F] | (iiii) |
| 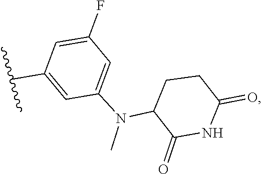 [SMILES: *c1cc(F)cc(N(C)C2CCC(=O)NC2=O)c1] | (jjjj) |
| 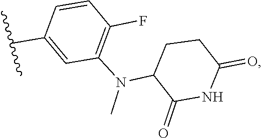 [SMILES: *c1ccc(F)c(N(C)C2CCC(=O)NC2=O)c1] | (kkkk) |
| 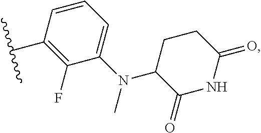 [SMILES: *c1cccc(N(C)C2CCC(=O)NC2=O)c1F] | (llll) |
| 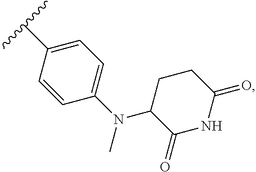 [SMILES: *c1ccc(N(C)C2CCC(=O)NC2=O)cc1] | (mmmm) |
| 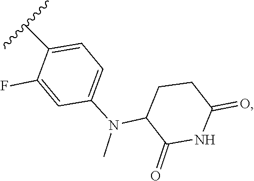 [SMILES: *c1ccc(N(C)C2CCC(=O)NC2=O)cc1F] | (nnnn) |
| 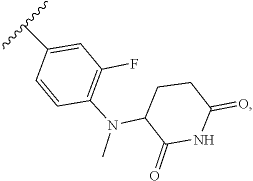 [SMILES: *c1ccc(N(C)C2CCC(=O)NC2=O)c(F)c1] | (oooo) |
| 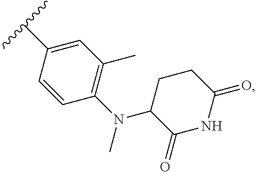 [SMILES: *c1ccc(N(C)C2CCC(=O)NC2=O)c(C)c1] | (pppp) |
| 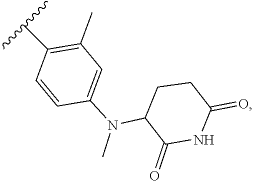 [SMILES: *c1ccc(N(C)C2CCC(=O)NC2=O)cc1C] | (qqqq) |
| 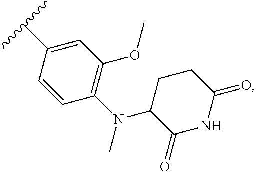 [SMILES: *c1ccc(N(C)C2CCC(=O)NC2=O)c(OC)c1] | (rrrr) |
| 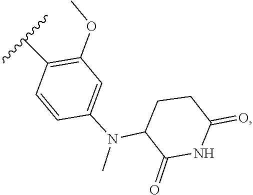 [SMILES: *c1ccc(N(C)C2CCC(=O)NC2=O)cc1OC] | (ssss) |
| 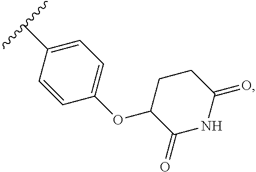 [SMILES: *c1ccc(OC2CCC(=O)NC2=O)cc1] | (tttt) |
| 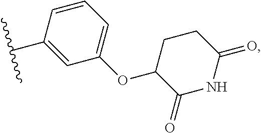 [SMILES: *c1cccc(OC2CCC(=O)NC2=O)c1] | (uuuu) |
| 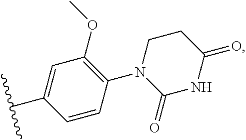 [SMILES: *c1ccc(N2CCC(=O)NC2=O)c(OC)c1] | (vvvv) |
| 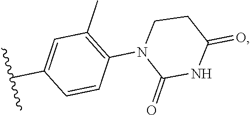 [SMILES: *c1ccc(N2CCC(=O)NC2=O)c(C)c1] | (wwww) |
| 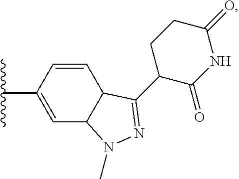 [SMILES: *C1=CC2C(C=C1)C(C1CCC(=O)NC1=O)=NN2C] | (xxxx) |
| 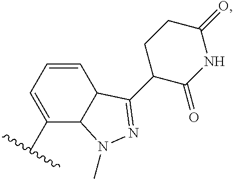 [SMILES: *C1=CC=CC2C(C3CCC(=O)NC3=O)=NN(C)C12] | (yyyy) |
| 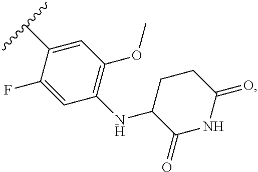 [SMILES: *c1cc(OC)c(NC2CCC(=O)NC2=O)cc1F] | (zzzz) |
| 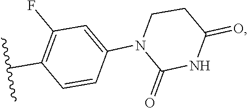 [SMILES: *c1ccc(N2CCC(=O)NC2=O)cc1F] | (aaaaa) |
| 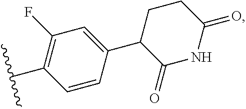 [SMILES: *c1ccc(C2CCC(=O)NC2=O)cc1F] | (bbbbb) |
| 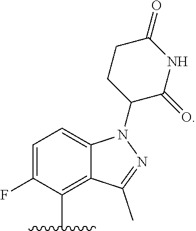 [SMILES: *c1c(F)ccc2c1c(C)nn2C1CCC(=O)NC1=O] | (cccccc) |
| or any of the LBM disclosed herein. | |

TABLE B
Exemplified Linkers (L)

| | |
|---|---|
| 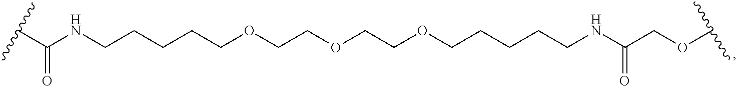 [SMILES: *OCC(=O)NCCCCCOCCOCCOCCCCCNC(*)=O] | (1) |
| 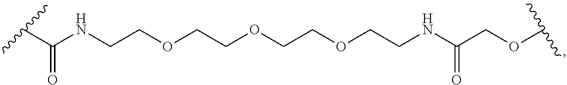 [SMILES: *OCC(=O)NCCOCCOCCOCCNC(*)=O] | (2) |
| 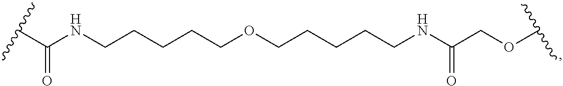 [SMILES: *OCC(=O)NCCCCCOCCCCCNC(*)=O] | (3) |
| 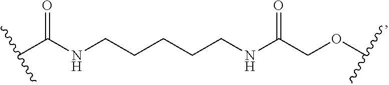 [SMILES: *OCC(=O)NCCCCCNC(*)=O] | (4) |
| 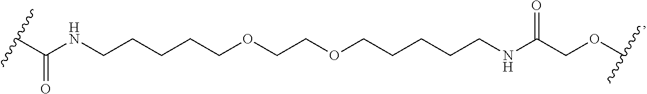 [SMILES: *OCC(=O)NCCCCCOCCOCCCCCNC(*)=O] | (5) |
| 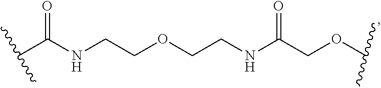 [SMILES: *OCC(=O)NCCOCCNC(*)=O] | (6) |
| 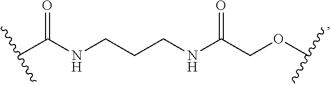 [SMILES: *OCC(=O)NCCCNC(*)=O] | (7) |
| 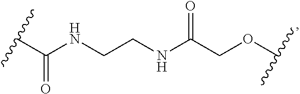 [SMILES: *OCC(=O)NCCNC(*)=O] | (8) |
| 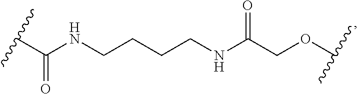 [SMILES: *OCC(=O)NCCCCNC(*)=O] | (9) |
| 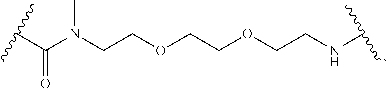 [SMILES: *NCCOCCOCCN(C)C(*)=O] | (10) |
| 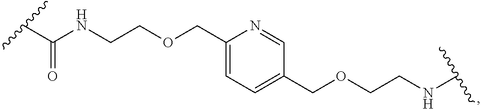 [SMILES: *NCCOCc1ccc(COCCNC(*)=O)nc1] | (11) |
| 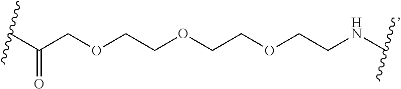 [SMILES: *NCCOCCOCCOCC(*)=O] | (12) |
| 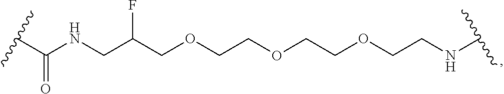 [SMILES: *NCCOCCOCCOCC(F)CNC(*)=O] | (13) |
| 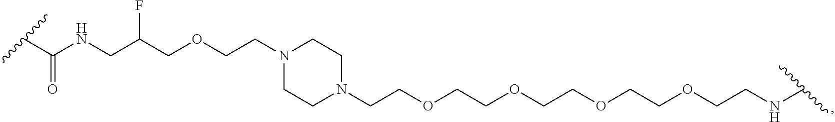 [SMILES: *NCCOCCOCCOCCOCCN1CCN(CCOCC(F)CNC(*)=O)CC1] | (14) |
| 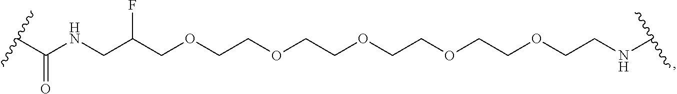 [SMILES: *NCCOCCOCCOCCOCCOCC(F)CNC(*)=O] | (15) |
| 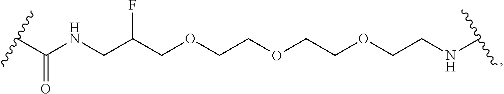 [SMILES: *NCCOCCOCCOCC(F)CNC(*)=O] | (16) |
| 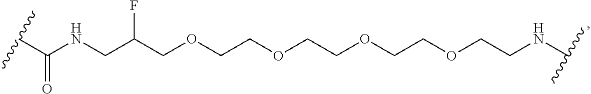 [SMILES: *NCCOCCOCCOCCOCC(F)CNC(*)=O] | (17) |
| 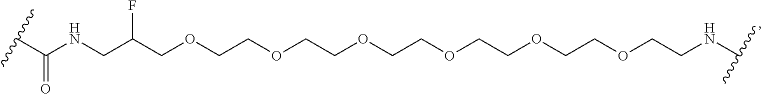 [SMILES: *NCCOCCOCCOCCOCCOCCOCC(F)CNC(*)=O] | (18) |
| 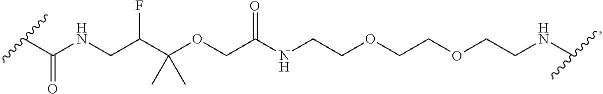 [SMILES: *NCCOCCOCCNC(=O)COC(C)(C)C(F)CNC(*)=O] | (19) |
| 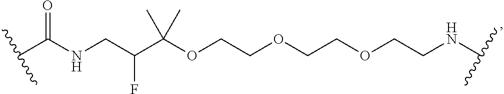 [SMILES: *NCCOCCOCCOC(C)(C)C(F)CNC(*)=O] | (20) |
| 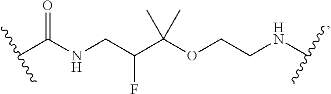 [SMILES: *NCCOC(C)(C)C(F)CNC(*)=O] | (21) |
| 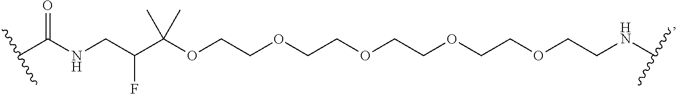 [SMILES: *NCCOCCOCCOCCOCCOC(C)(C)C(F)CNC(*)=O] | (22) |
| 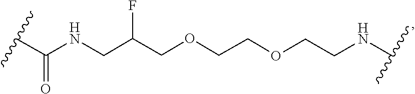 [SMILES: *NCCOCCOCC(F)CNC(*)=O] | (23) |
| 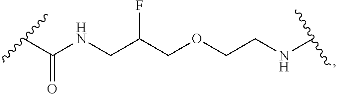 [SMILES: *NCCOCC(F)CNC(*)=O] | (24) |
| 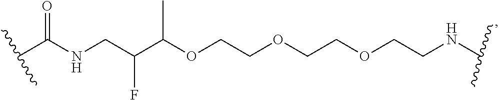 [SMILES: *NCCOCCOCCOC(C)C(F)CNC(*)=O] | (25) |
| 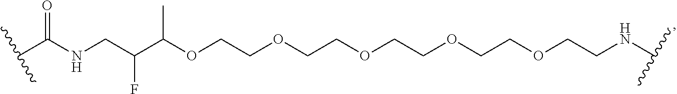 [SMILES: *NCCOCCOCCOCCOCCOC(C)C(F)CNC(*)=O] | (26) |
| 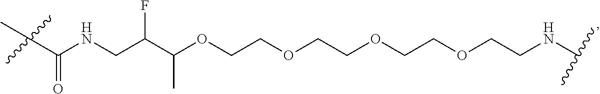 [SMILES: *NCCOCCOCCOCCOC(C)C(F)CNC(*)=O] | (27) |
| 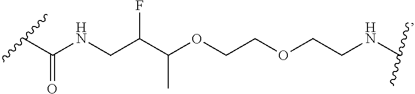 [SMILES: *NCCOCCOC(C)C(F)CNC(*)=O] | (28) |
| 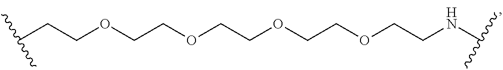 [SMILES: *CCOCCOCCOCCOCCN*] | (29) |
| 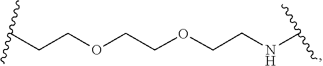 [SMILES: *CCOCCOCCN*] | (30) |
| 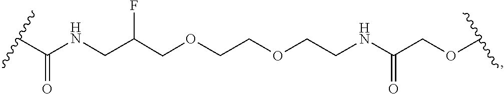 [SMILES: *OCC(=O)NCCOCCOCC(F)CNC(*)=O] | (31) |
| 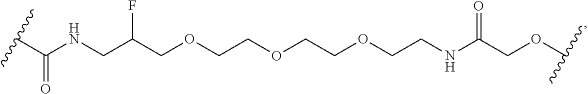 [SMILES: *OCC(=O)NCCOCCOCCOCC(F)CNC(*)=O] | (32) |
| 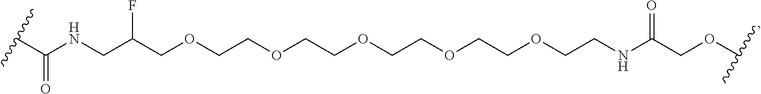 [SMILES: *OCC(=O)NCCOCCOCCOCCOCCOCC(F)CNC(*)=O] | (33) |
| 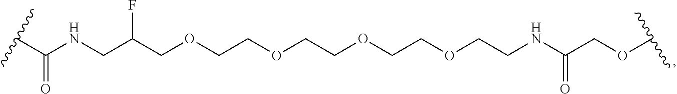 [SMILES: *OCC(=O)NCCOCCOCCOCCOCC(F)CNC(*)=O] | (34) |
| 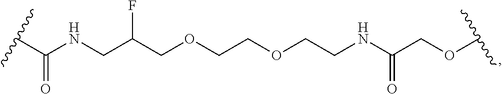 [SMILES: *OCC(=O)NCCOCCOCC(F)CNC(*)=O] | (35) |
| 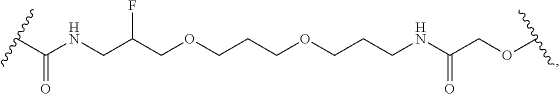 [SMILES: *OCC(=O)NCCCOCCCOCC(F)CNC(*)=O] | (36) |
| 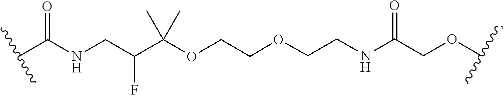 [SMILES: *OCC(=O)NCCOCCOC(C)(C)C(F)CNC(*)=O] | (37) |
| 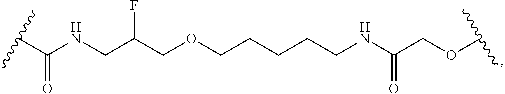 [SMILES: *OCC(=O)NCCCCCOCC(F)CNC(*)=O] | (38) |
| 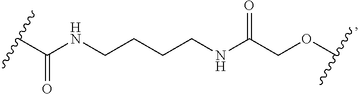 [SMILES: *OCC(=O)NCCCCNC(*)=O] | (39) |
| 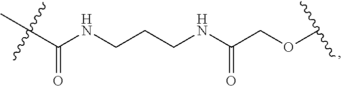 [SMILES: *OCC(=O)NCCCNC(*)=O] | (40) |
| 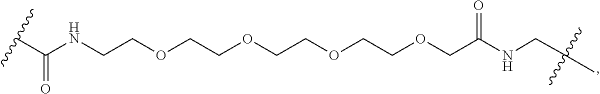 [SMILES: *CNC(=O)COCCOCCOCCOCCNC(*)=O] | (41) |
| 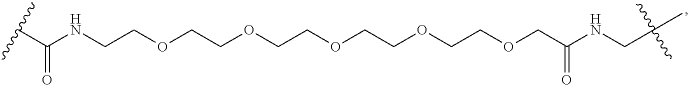 [SMILES: *CNC(=O)COCCOCCOCCOCCOCCNC(*)=O] | (42) |
| 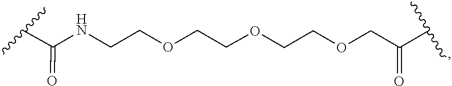 [SMILES: *C(=O)COCCOCCOCCNC(*)=O] | (43) |
| 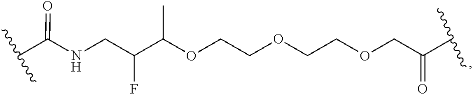 [SMILES: *C(=O)COCCOCCOC(C)C(F)CNC(*)=O] | (44) |
| 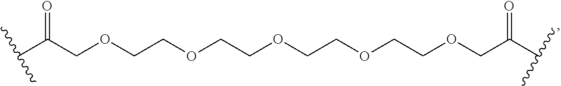 [SMILES: *C(=O)COCCOCCOCCOCCOCC(*)=O] | (45) |
| 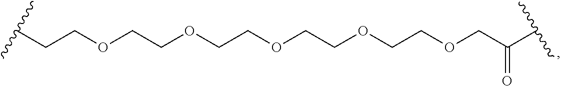 [SMILES: *CCOCCOCCOCCOCCOCC(*)=O] | (46) |
| 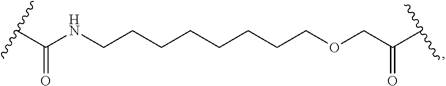 [SMILES: *C(=O)COCCCCCCCCNC(*)=O] | (47) |
| 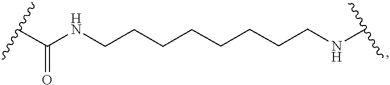 [SMILES: *NCCCCCCCCNC(*)=O] | (49) |
| 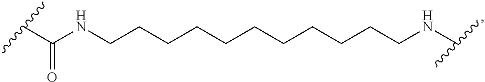 [SMILES: *NCCCCCCCCCCCNC(*)=O] | (50) |
| 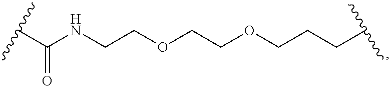 [SMILES: *CCCOCCOCCNC(*)=O] | (51) |
| 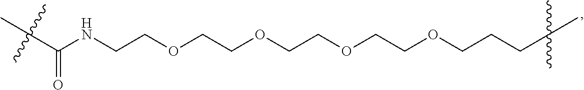 [SMILES: *CCCOCCOCCOCCOCCNC(*)=O] | (52) |
| 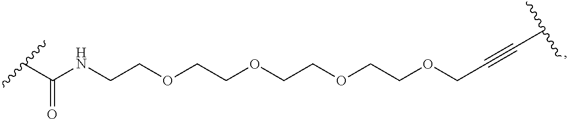 [SMILES: *C#CCOCCOCCOCCOCCNC(*)=O] | (53) |
| 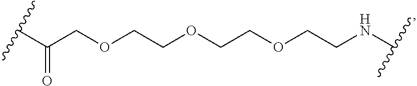 [SMILES: *NCCOCCOCCOCC(*)=O] | (54) |
| 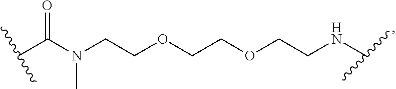 [SMILES: *NCCOCCOCCN(C)C(*)=O] | (55) |
| 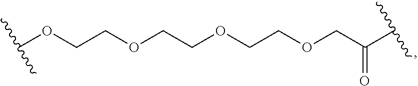 [SMILES: *OCCOCCOCCOCC(*)=O] | (56) |
| 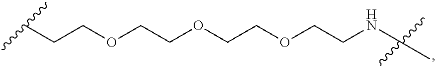 [SMILES: *CCOCCOCCOCCN*] | (57) |
| 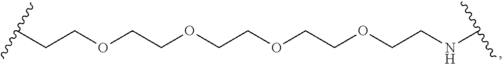 [SMILES: *CCOCCOCCOCCOCCN*] | (58) |
| 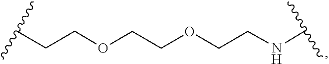 [SMILES: *CCOCCOCCN*] | (59) |
| 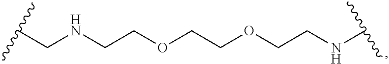 [SMILES: *CNCCOCCOCCN*] | (60) |
| 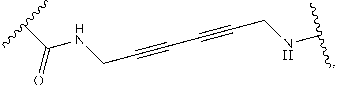 [SMILES: *NCC#CC#CCNC(*)=O] | (61) |
| 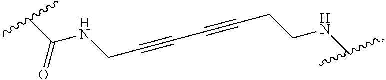 [SMILES: *NCCC#CC#CCNC(*)=O] | (62) |
| 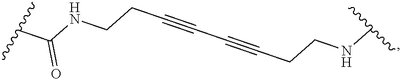 [SMILES: *NCCC#CC#CCCNC(*)=O] | (63) |
| 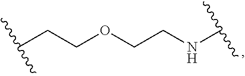 [SMILES: *CCOCCN*] | (64) |
| 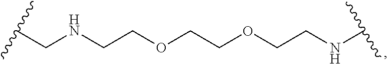 [SMILES: *CNCCOCCOCCN*] | (65) |
| 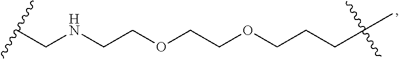 [SMILES: *CCCOCCOCCNC*] | (66) |
| 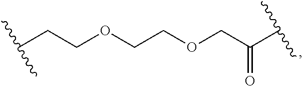 [SMILES: *CCOCCOCC(*)=O] | (67) |
| 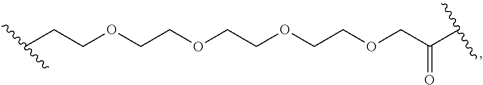 [SMILES: *CCOCCOCCOCCOCC(*)=O] | (68) |
| 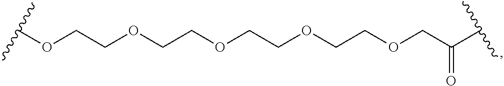 [SMILES: *OCCOCCOCCOCCOCC(*)=O] | (69) |
| 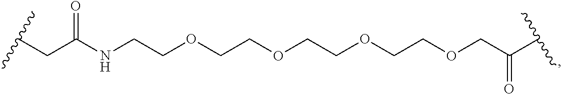 [SMILES: *CC(=O)NCCOCCOCCOCCOCC(*)=O] | (70) |
| 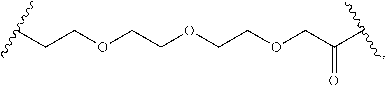 [SMILES: *CCOCCOCCOCC(*)=O] | (71) |
| 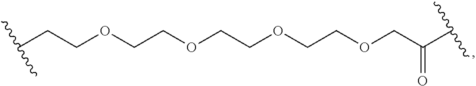 [SMILES: *CCOCCOCCOCCOCC(*)=O] | (72) |
| 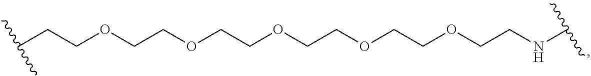 [SMILES: *CCOCCOCCOCCOCCOCCN*] | (73) |
| 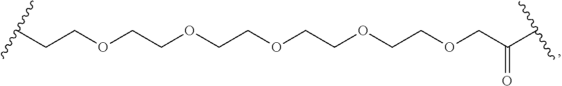 [SMILES: *CCOCCOCCOCCOCCOCC(*)=O] | (74) |
| 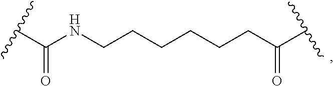 [SMILES: *C(=O)CCCCCCNC(*)=O] | (75) |
| 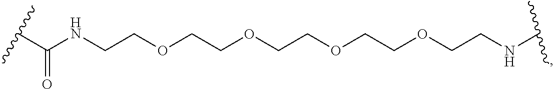 [SMILES: *NCCOCCOCCOCCOCCNC(*)=O] | (76) |
| 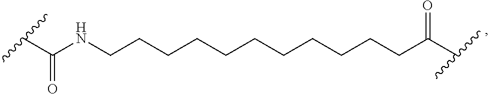 [SMILES: *C(=O)CCCCCCCCCCCNC(*)=O] | (77) |
| 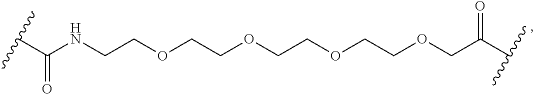 [SMILES: *C(=O)COCCOCCOCCOCCNC(*)=O] | (78) |
| 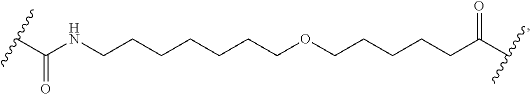 [SMILES: *C(=O)CCCCCOCCCCCCCNC(*)=O] | (79) |
| 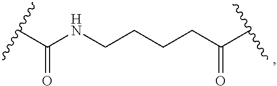 [SMILES: *C(=O)CCCCNC(*)=O] | (80) |
| 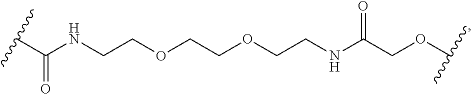 [SMILES: *OCC(=O)NCCOCCOCCNC(*)=O] | (81) |
| 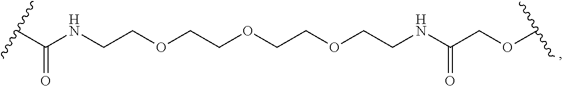 [SMILES: *OCC(=O)NCCOCCOCCOCCNC(*)=O] | (82) |
| 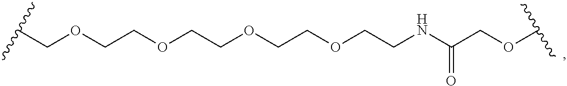 [SMILES: *COCCOCCOCCOCCNC(=O)CO*] | (83) |
| 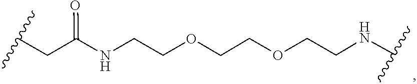 [SMILES: *CC(=O)NCCOCCOCCN*] | (84) |
| 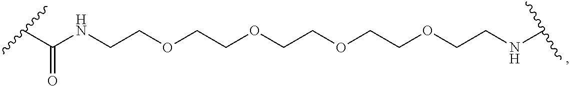 [SMILES: *NCCOCCOCCOCCOCCNC(*)=O] | (85) |
| 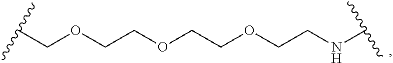 [SMILES: *COCCOCCOCCN*] | (86) |
| 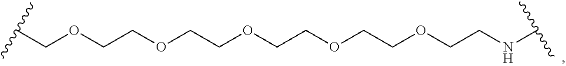 [SMILES: *COCCOCCOCCOCCOCCN*] | (87) |
| 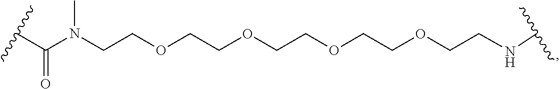 [SMILES: *NCCOCCOCCOCCOCCN(C)C(*)=O] | (88) |
| 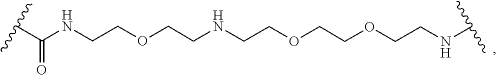 [SMILES: *NCCOCCOCCNCCOCCNC(*)=O] | (89) |
| 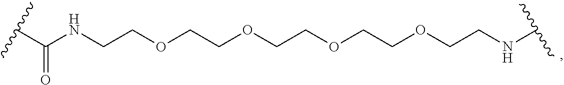 [SMILES: *NCCOCCOCCOCCOCCNC(*)=O] | (90) |
| 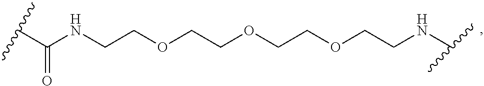 [SMILES: *NCCOCCOCCOCCNC(*)=O] | (91) |
| 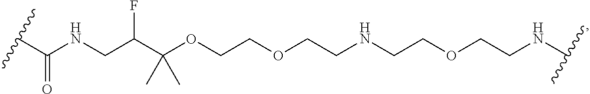 [SMILES: *NCCOCCNCCOCCOC(C)(C)C(F)CNC(*)=O ; ","] | (92) |
| 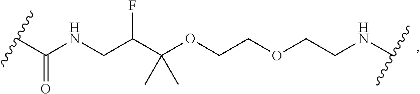 [SMILES: *NCCOCCOC(C)(C)C(F)CNC(*)=O] | (93) |
| 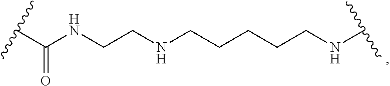 [SMILES: *NCCCCCNCCNC(*)=O] | (94) |
| 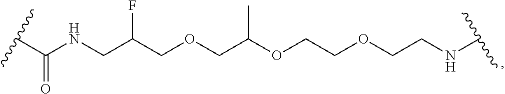 [SMILES: *NCCOCCOC(C)COCC(F)CNC(*)=O] | (95) |
| 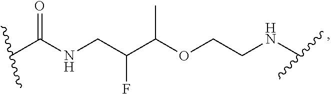 [SMILES: *NCCOC(C)C(F)CNC(*)=O] | (96) |
| 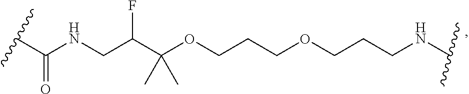 [SMILES: *NCCCOCCCOC(C)(C)C(F)CNC(*)=O] | (97) |
| 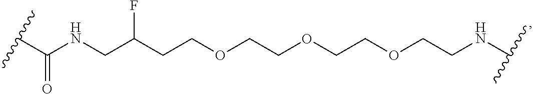 [SMILES: *NCCOCCOCCOCCC(F)CNC(*)=O ; ","] | (98) |
| 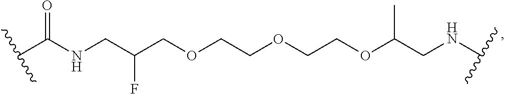 [SMILES: *NCC(C)OCCOCCOCC(F)CNC(*)=O] | (99) |
| 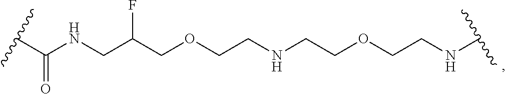 [SMILES: *NCCOCCNCCOCC(F)CNC(*)=O] | (100) |
| 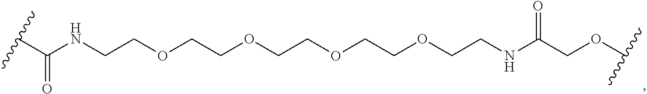 [SMILES: *OCC(=O)NCCOCCOCCOCCOCCNC(*)=O] | (101) |
| 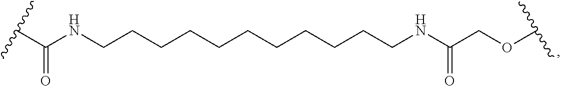 [SMILES: *OCC(=O)NCCCCCCCCCCCNC(*)=O] | (102) |
| 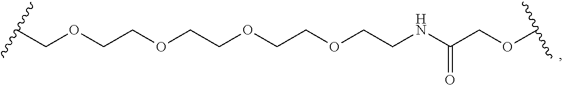 [SMILES: *COCCOCCOCCOCCNC(=O)CO*] | (103) |
| 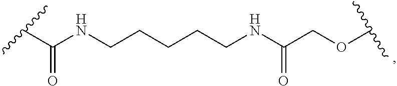 [SMILES: *OCC(=O)NCCCCCNC(*)=O] | (104) |
| 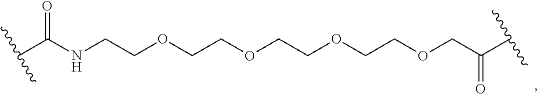 [SMILES: *C(=O)COCCOCCOCCOCCNC(*)=O] | (105) |
| 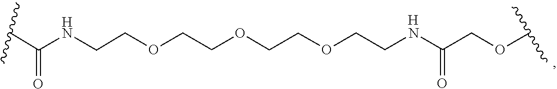 | (106) |
| 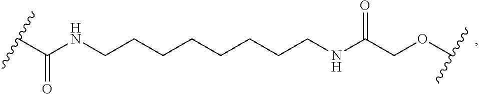 [SMILES: *OCC(=O)NCCCCCCCCNC(*)=O] | (107) |
| 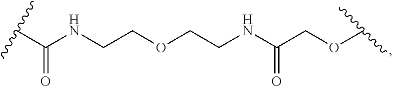 [SMILES: *OCC(=O)NCCOCCNC(*)=O] | (108) |
| 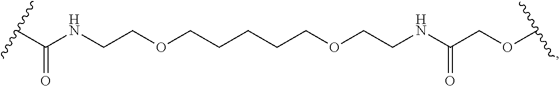 [SMILES: *OCC(=O)NCCOCCCCCOCCNC(*)=O] | (109) |
| 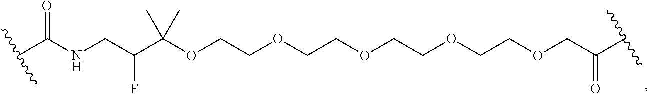 [SMILES: *C(=O)COCCOCCOCCOCCOC(C)(C)C(F)CNC(*)=O] | (110) |
| 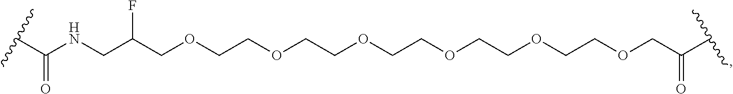 [SMILES: *C(=O)COCCOCCOCCOCCOCCOCC(F)CNC(*)=O] | (111) |
| 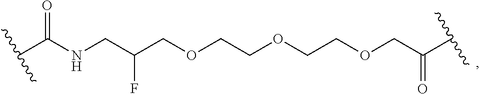 [SMILES: *C(=O)COCCOCCOCC(F)CNC(*)=O] | (112) |
| 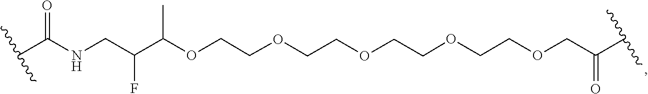 [SMILES: *C(=O)COCCOCCOCCOCCOC(C)C(F)CNC(*)=O] | (113) |
| 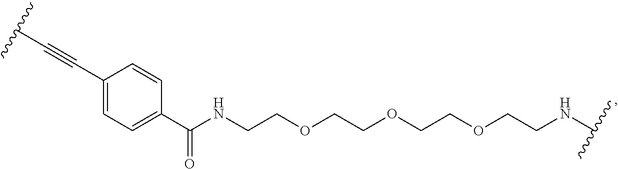 [SMILES: *C#Cc1ccc(C(=O)NCCOCCOCCOCCN*)cc1] | (114) |
| 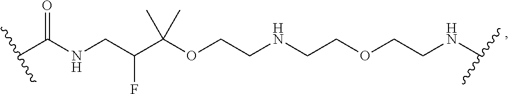 [SMILES: *NCCOCCNCCOC(C)(C)C(F)CNC(*)=O] | (115) |
| 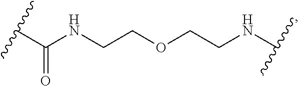 [SMILES: *NCCOCCNC(*)=O] | (116) |
| 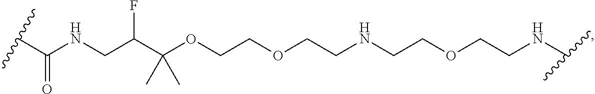 [SMILES: *NCCOCCNCCOCCOC(C)(C)C(F)CNC(*)=O] | (117) |
| 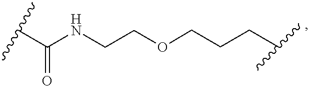 [SMILES: *CCCOCCNC(*)=O] | (118) |
| 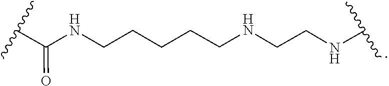 [SMILES: *NCCNCCCCCNC(*)=O] | (119) |
| 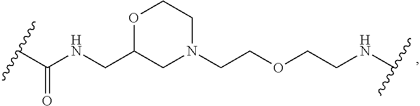 [SMILES: *NCCOCCN1CCOC(CNC(*)=O)C1] | |
| 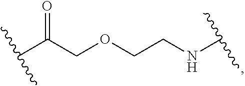 [SMILES: *NCCOCC(*)=O] | (120) |
| 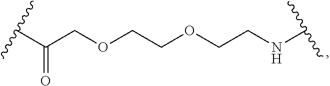 [SMILES: *NCCOCCOCC(*)=O] | |
| 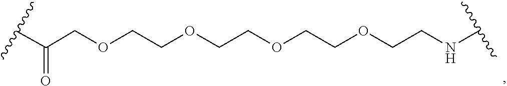 [SMILES: *NCCOCCOCCOCCOCC(*)=O] | (121) |
| 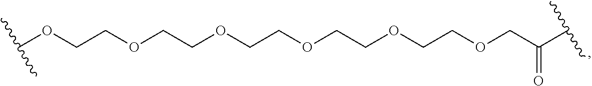 [SMILES: *OCCOCCOCCOCCOCCOCC(*)=O] | (122) |
| 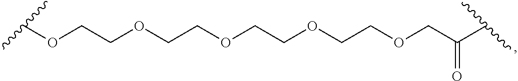 [SMILES: *OCCOCCOCCOCCOCC(*)=O] | (123) |
| 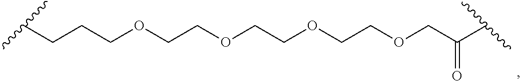 [SMILES: *CCCOCCOCCOCCOCC(*)=O] | (124) |
| 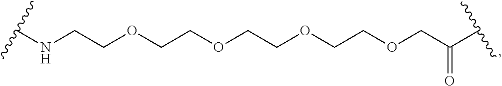 [SMILES: *NCCOCCOCCOCCOCC(*)=O] | (125) |
| 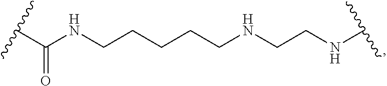 [SMILES: *NCCNCCCCCNC(*)=O] | (126) |
| 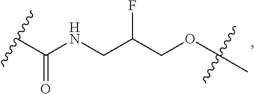 [SMILES: *OCC(F)CNC(*)=O] | (127) |
| 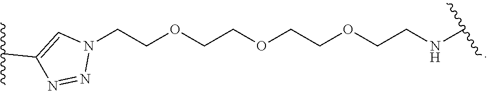 [SMILES: *NCCOCCOCCOCCn1cc(*)nn1] | |
| 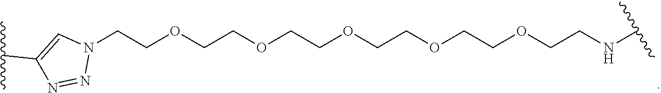 [SMILES: *NCCOCCOCCOCCOCCOCCn1cc(*)nn1] | (128) |
| 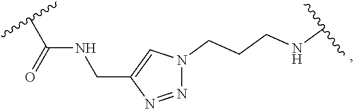 [SMILES: *NCCCn1cc(CNC(*)=O)nn1] | (129) |
| 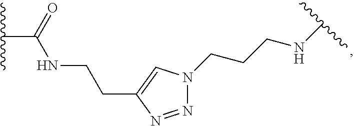 [SMILES: *NCCCn1cc(CCNC(*)=O)nn1] | (130) |
| 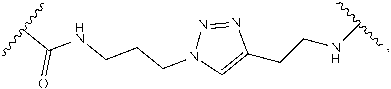 [SMILES: *NCCc1cn(CCCNC(*)=O)nn1] | (131) |
| 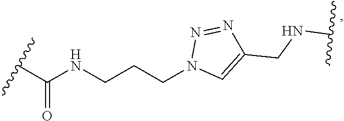 [SMILES: *NCc1cn(CCCNC(*)=O)nn1] | (132) |
| 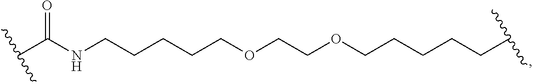 [SMILES: *CCCCCOCCOCCCCCNC(*)=O] | (133) |
| 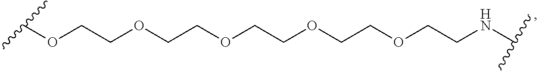 [SMILES: *NCCOCCOCCOCCOCCO*] | (134) |
| 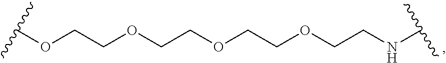 [SMILES: *NCCOCCOCCOCCO*] | (135) |
| 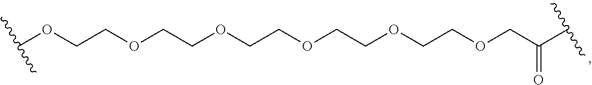 [SMILES: *OCCOCCOCCOCCOCCOCC(*)=O] | (136) |
| 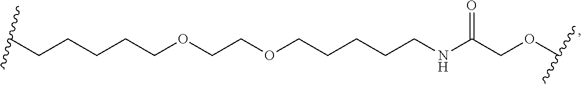 [SMILES: *CCCCCOCCOCCCCCNC(=O)CO*] | (137) |
| 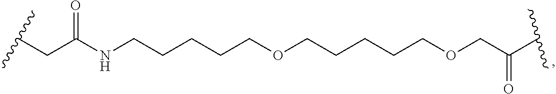 [SMILES: *CC(=O)NCCCCCOCCCCCOCC(*)=O] | (138) |
| 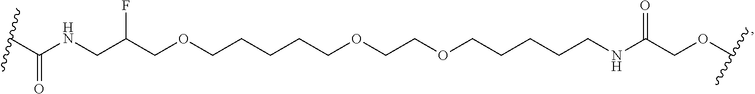 [SMILES: *OCC(=O)NCCCCCOCCOCCCCCOCC(F)CNC(*)=O] | (139) |
| 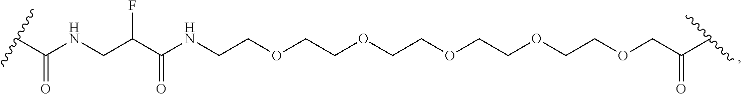 [SMILES: *C(=O)COCCOCCOCCOCCOCCNC(=O)C(F)CNC(*)=O] | (140) |
| 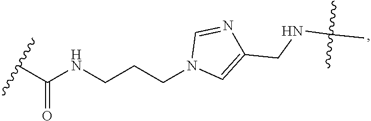 [SMILES: *NCc1cn(CCCNC(*)=O)cn1] | (141) |
| 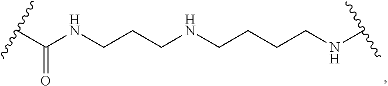 [SMILES: *NCCCCNCCCNC(*)=O] | (142) |
| 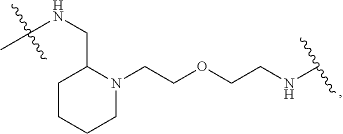 [SMILES: *NCCOCCN1CCCCC1CN* ; ","] | (143) |
| 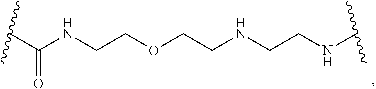 [SMILES: *NCCNCCOCCNC(*)=O] | (144) |
| 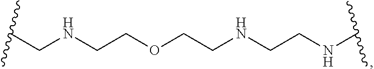 [SMILES: *CNCCOCCNCCN* ; ","] | (145) |
| 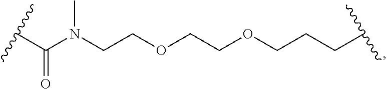 [SMILES: *CCCOCCOCCN(C)C(*)=O] | (146) |
| 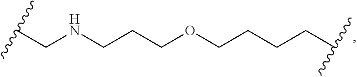 [SMILES: *CCCCOCCCNC*] | (147) |
| 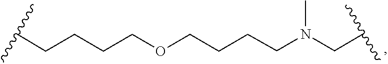 [SMILES: *CCCCOCCCCN(C)C*] | (148) |
| 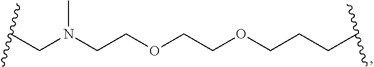 [SMILES: *CCCOCCOCCN(C)C*] | (149) |
| 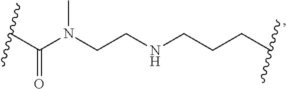 [SMILES: *CCCNCCN(C)C(*)=O] | (150) |
| 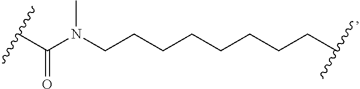 [SMILES: *CCCCCCCCN(C)C(*)=O ; ","] | (151) |
| 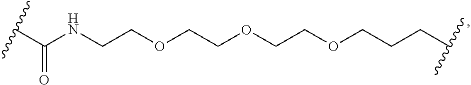 [SMILES: *CCCOCCOCCOCCNC(*)=O] | (152) |
| 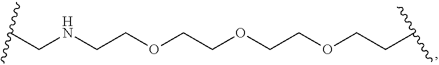 [SMILES: *CCOCCOCCOCCNC*] | (153) |
| 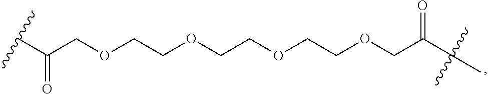 [SMILES: *C(=O)COCCOCCOCCOCC(*)=O ; ","] | (154) |
| 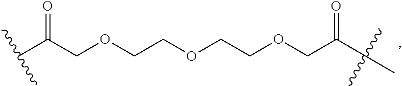 [SMILES: *C(=O)COCCOCCOCC(*)=O] | (155) |
| 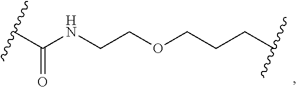 [SMILES: *CCCOCCNC(*)=O] | (156) |
| 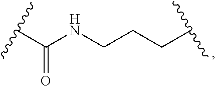 [SMILES: *CCCNC(*)=O] | (157) |
| 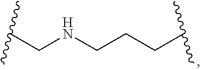 [SMILES: *CCCNC*] | (158) |
| 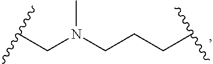 [SMILES: *CCCN(C)C*] | (159) |
| 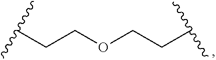 [SMILES: *CCOCC*] | (160) |
| 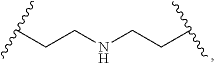 [SMILES: *CCNCC* ; ","] | (161) |
| 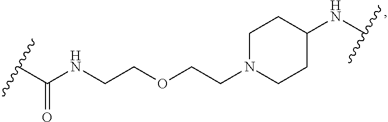 [SMILES: *NC1CCN(CCOCCNC(*)=O)CC1] | (162) |
| 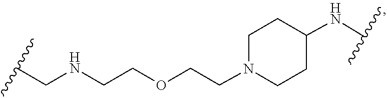 [SMILES: *CNCCOCCN1CCC(N*)CC1] | (163) |
| 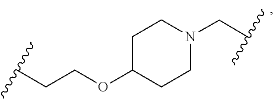 [SMILES: *CCOC1CCN(C*)CC1] | (164) |
| 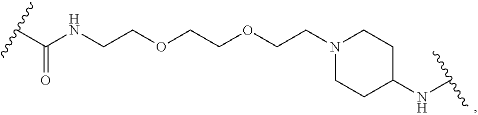 [SMILES: *NC1CCN(CCOCCOCCNC(*)=O)CC1] | (165) |
| 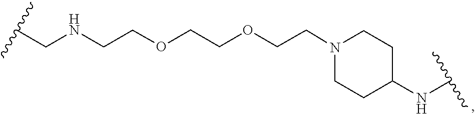 [SMILES: *CNCCOCCOCCN1CCC(N*)CC1 ; ","] | (166) |
| 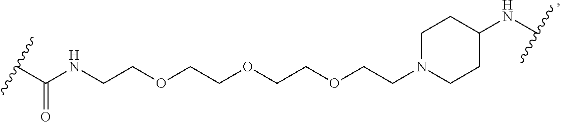 [SMILES: "," ; *NC1CCN(CCOCCOCCOCCNC(*)=O)CC1] | (167) |
| 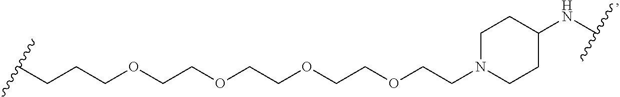 [SMILES: "," ; *CCCOCCOCCOCCOCCN1CCC(N*)CC1] | (168) |
| 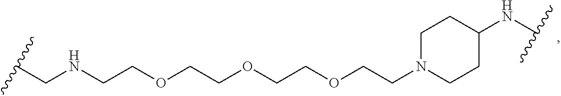 [SMILES: *CNCCOCCOCCOCCN1CCC(N*)CC1] | (169) |
| 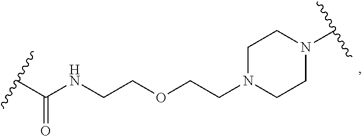 [SMILES: *C(=O)NCCOCCN1CCN(*)CC1] | (170) |
| 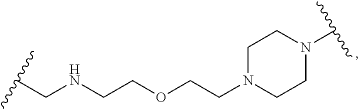 [SMILES: *CNCCOCCN1CCN(*)CC1] | (171) |
| 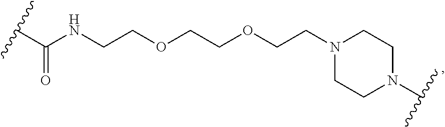 [SMILES: *C(=O)NCCOCCOCCN1CCN(*)CC1] | (172) |
| 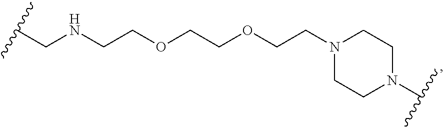 [SMILES: *CNCCOCCOCCN1CCN(*)CC1] | (173) |
| 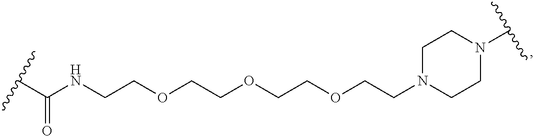 [SMILES: *C(=O)NCCOCCOCCOCCN1CCN(*)CC1] | (174) |
| 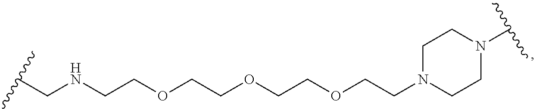 [SMILES: *CNCCOCCOCCOCCN1CCN(*)CC1 ; ","] | (175) |
| 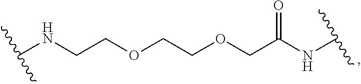 [SMILES: *NCCOCCOCC(=O)N*] | (176) |
| 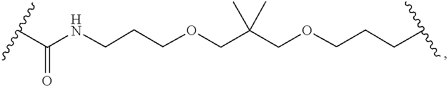 [SMILES: *CCCOCC(C)(C)COCCCNC(*)=O ; ","] | (177) |
| 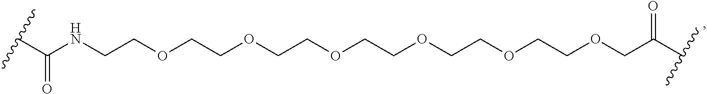 [SMILES: *C(=O)COCCOCCOCCOCCOCCOCCNC(*)=O ; ","] | (178) |
| 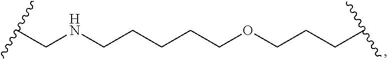 [SMILES: *CCCOCCCCCNC*] | (179) |
| 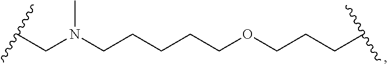 [SMILES: *CCCOCCCCCN(C)C*] | (180) |
| 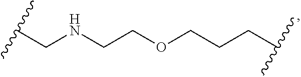 [SMILES: *CCCOCCNC*] | (181) |
| 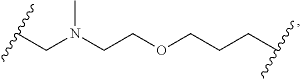 [SMILES: *CCCOCCN(C)C*] | (182) |
| 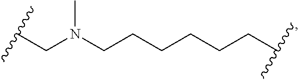 [SMILES: *CCCCCCN(C)C*] | (183) |
| 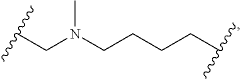 [SMILES: *CCCCN(C)C*] | (184) |
| 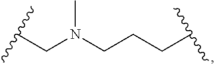 [SMILES: *CCCN(C)C*] | (185) |
| 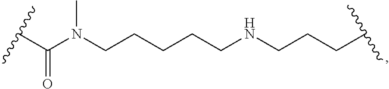 [SMILES: *CCCNCCCCCN(C)C(*)=O] | (186) |
| 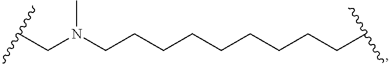 [SMILES: *CCCCCCCCCN(C)C*] | (187) |
| 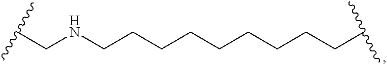 [SMILES: *CCCCCCCCCNC*] | (188) |
| 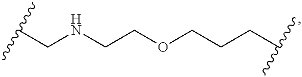 [SMILES: *CCCOCCNC*] | (189) |
| 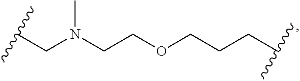 [SMILES: *CCCOCCN(C)C*] | (190) |
| 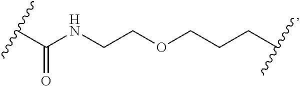 [SMILES: *CCCOCCNC(*)=O] | (191) |
| 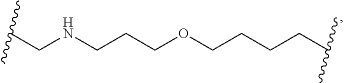 [SMILES: *CCCCOCCCNC*] | (192) |
| 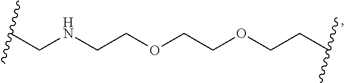 [SMILES: *CCOCCOCCNC*] | (193) |
| 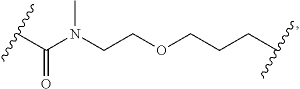 [SMILES: *CCCOCCN(C)C(*)=O] | (194) |
| 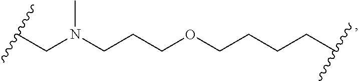 [SMILES: *CCCCOCCCN(C)C*] | (195) |
| 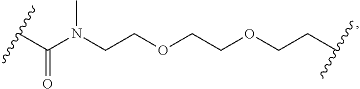 [SMILES: *CCOCCOCCN(C)C(*)=O] | (196) |
| 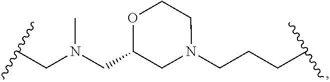 [SMILES: *CCCN1CCO[C@@H](CN(C)C*)C1] | (197) |
| 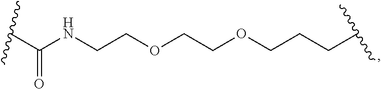 [SMILES: *CCCOCCOCCNC(*)=O] | (198) |
| 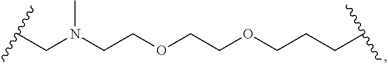 [SMILES: *CCCOCCOCCN(C)C*] | (199) |
| 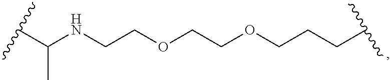 [SMILES: *CCCOCCOCCNC(*)C] | (200) |
| 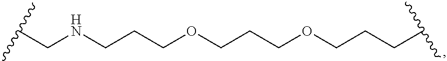 [SMILES: *CCCOCCCOCCCNC*] | (201) |
| 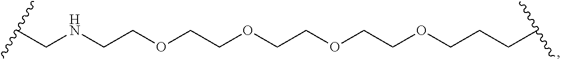 [SMILES: *CCCOCCOCCOCCOCCNC*] | (202) |
| 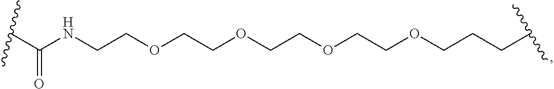 [SMILES: *CCCOCCOCCOCCOCCNC(*)=O] | (203) |
| 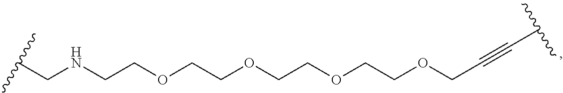 [SMILES: *C#CCOCCOCCOCCOCCNC*] | (204) |
| 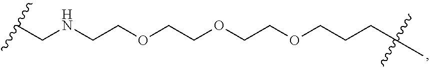 [SMILES: *CCCOCCOCCOCCNC*] | (205) |
| 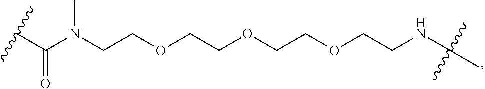 [SMILES: *NCCOCCOCCOCCN(C)C(*)=O] | (206) |
| 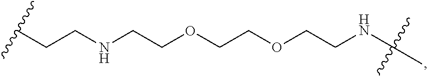 [SMILES: *CCNCCOCCOCCN*] | (207) |
| 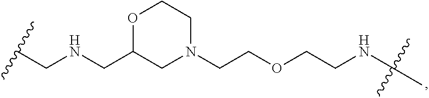 [SMILES: *CNCC1CN(CCOCCN*)CCO1] | (208) |
| 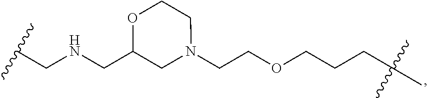 [SMILES: *CCCOCCN1CCOC(CNC*)C1] | (209) |
| 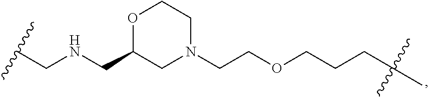 [SMILES: *CCCOCCN1CCO[C@H](CNC*)C1] | (210) |
| 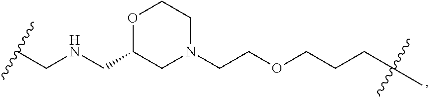 [SMILES: *CCCOCCN1CCO[C@@H](CNC*)C1] | (211) |
| 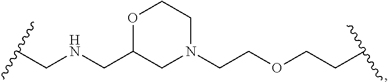 [SMILES: *CCOCCN1CCOC(CNC*)C1] | (212) |
| 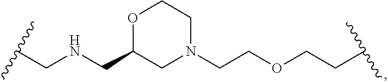 [SMILES: *CCOCCN1CCO[C@H](CNC*)C1] | (213) |
| 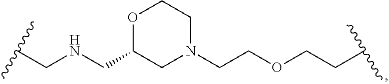 [SMILES: *CCOCCN1CCO[C@@H](CNC*)C1] | (214) |
| 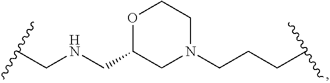 [SMILES: *CCCN1CCO[C@@H](CNC*)C1] | (215) |
| 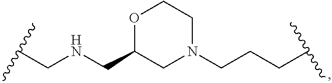 [SMILES: *CCCN1CCO[C@H](CNC*)C1] | (216) |
| 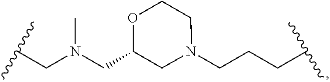 [SMILES: *CCCN1CCO[C@@H](CN(C)C*)C1] | (217) |
| 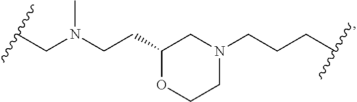 [SMILES: *CCCN1CCO[C@H](CCN(C)C*)C1] | (218) |
| 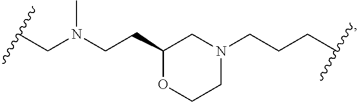 [SMILES: *CCCN1CCO[C@@H](CCN(C)C*)C1] | (219) |
| 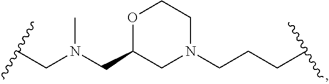 [SMILES: *CCCN1CCO[C@H](CN(C)C*)C1] | (220) |
| 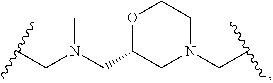 [SMILES: *CN(C)C[C@H]1CN(C*)CCO1] | (221) |
| 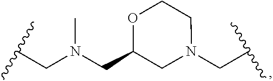 [SMILES: *CN(C)C[C@@H]1CN(C*)CCO1] | (222) |
| 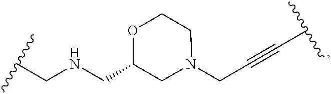 [SMILES: *C#CCN1CCO[C@@H](CNC*)C1] | (223) |
| 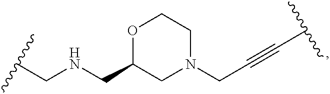 [SMILES: *C#CCN1CCO[C@H](CNC*)C1] | (224) |
| 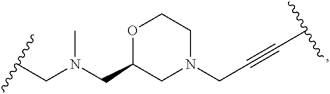 [SMILES: *C#CCN1CCO[C@H](CN(C)C*)C1] | (225) |
| 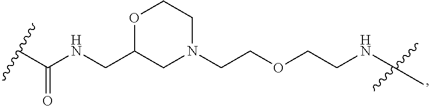 [SMILES: *NCCOCCN1CCOC(CNC(*)=O)C1] | (226) |
| 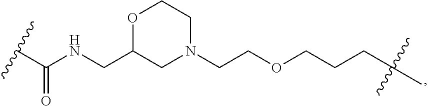 [SMILES: *CCCOCCN1CCOC(CNC(*)=O)C1] | (227) |
| 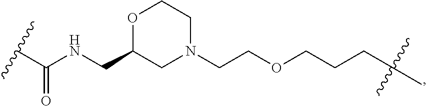 [SMILES: *CCCOCCN1CCO[C@H](CNC(*)=O)C1] | (228) |
| 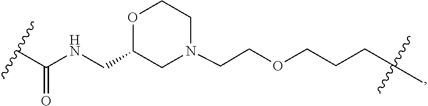 [SMILES: *CCCOCCN1CCO[C@@H](CNC(*)=O)C1] | (229) |
| 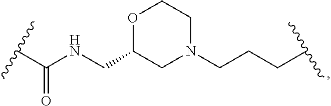 [SMILES: *CCCN1CCO[C@@H](CNC(*)=O)C1] | (230) |
| 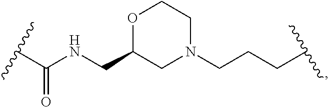 [SMILES: *CCCN1CCO[C@H](CNC(*)=O)C1] | (231) |
| 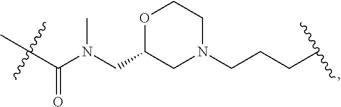 [SMILES: *CCCN1CCO[C@@H](CN(C)C(*)=O)C1] | (232) |
| 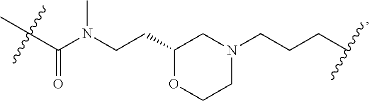 [SMILES: *CCCN1CCO[C@H](CCN(C)C(*)=O)C1] | (233) |
| 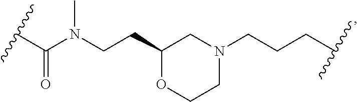 [SMILES: *CCCN1CCO[C@@H](CCN(C)C(*)=O)C1] | (234) |
| 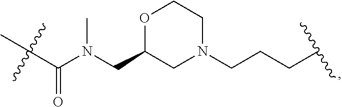 [SMILES: *CCCN1CCO[C@H](CN(C)C(*)=O)C1] | (235) |
| 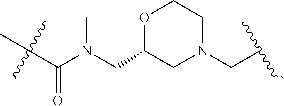 [SMILES: *CN1CCO[C@@H](CN(C)C(*)=O)C1] | (236) |
| 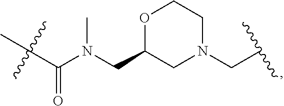 [SMILES: *CN1CCO[C@H](CN(C)C(*)=O)C1] | (237) |
| 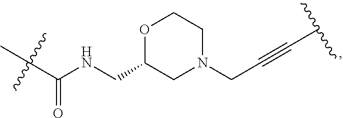 | (238) |
| 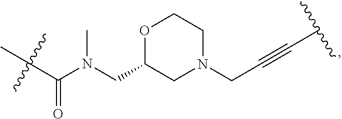 [SMILES: *C#CCN1CCO[C@@H](CN(C)C(*)=O)C1] | (239) |
| 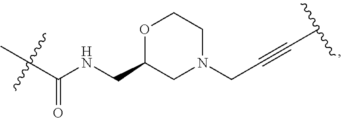 [SMILES: *C#CCN1CCO[C@H](CNC(*)=O)C1] | (240) |
| 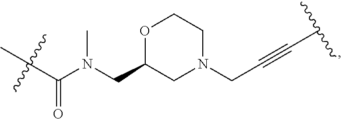 [SMILES: *C#CCN1CCO[C@H](CN(C)C(*)=O)C1] | (241) |
| 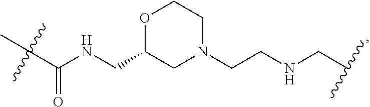 [SMILES: *CNCCN1CCO[C@@H](CNC(*)=O)C1] | (242) |
| 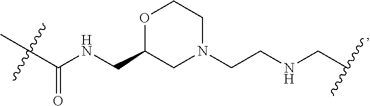 [SMILES: *CNCCN1CCO[C@H](CNC(*)=O)C1] | (243) |
| 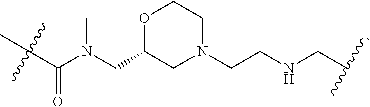 [SMILES: *CNCCN1CCO[C@@H](CN(C)C(*)=O)C1] | (244) |
| 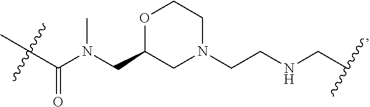 [SMILES: *CNCCN1CCO[C@H](CN(C)C(*)=O)C1] | (245) |
| 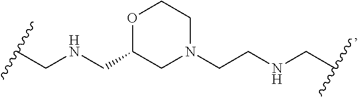 [SMILES: *CNCCN1CCO[C@@H](CNC*)C1] | (246) |
| 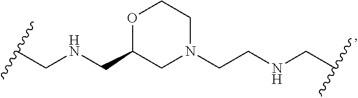 [SMILES: *CNCCN1CCO[C@H](CNC*)C1] | (247) |
| 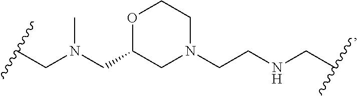 [SMILES: *CNCCN1CCO[C@@H](CN(C)C*)C1] | (248) |
| 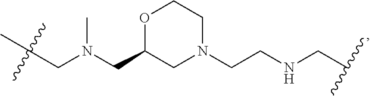 [SMILES: *CNCCN1CCO[C@H](CN(C)C*)C1] | (249) |
| 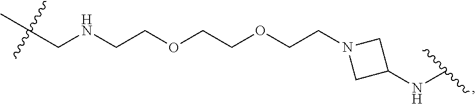 [SMILES: *CNCCOCCOCCN1CC(N*)C1] | (250) |
| 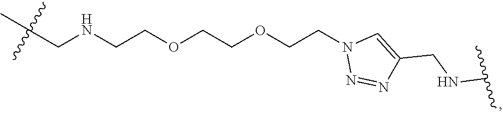 [SMILES: *CNCCOCCOCCn1cc(CN*)nn1] | (251) |
| 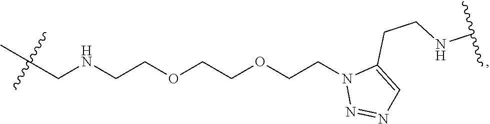 [SMILES: *CNCCOCCOCCn1nncc1CCN*] | (253) |
| 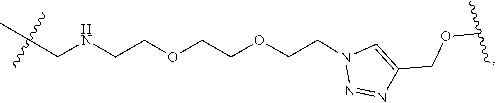 [SMILES: *CNCCOCCOCCn1cc(CO*)nn1] | (254) |
| 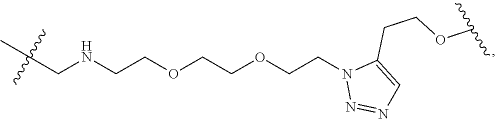 [SMILES: *CNCCOCCOCCn1nncc1CCO*] | (255) |
| 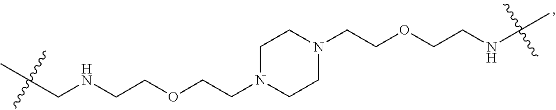 [SMILES: *CNCCOCCN1CCN(CCOCCN*)CC1] | (256) |
| 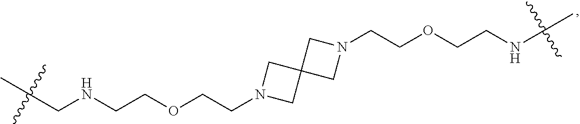 [SMILES: *CNCCOCCN1CC2(CN(CCOCCN*)C2)C1] | (257) |
| 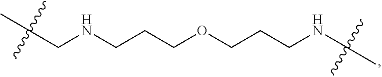 [SMILES: *CNCCCOCCCN*] | (258) |
| 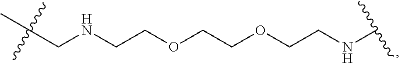 [SMILES: *CNCCOCCOCCN*] | (259) |
| 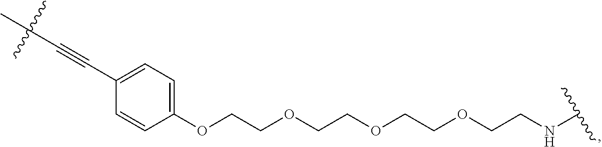 [SMILES: *C#Cc1ccc(OCCOCCOCCOCCN*)cc1] | (260) |
| 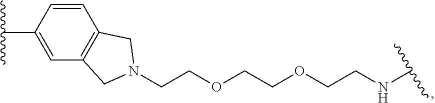 [SMILES: *NCCOCCOCCN1Cc2ccc(*)cc2C1] | (261) |
| 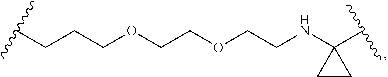 [SMILES: *CCCOCCOCCNC1(*)CC1] | (262) |
| 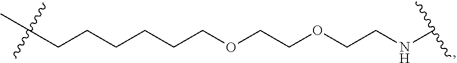 [SMILES: *CCCCCCOCCOCCN*] | (263) |
| 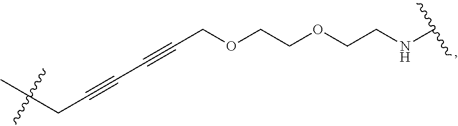 [SMILES: *CC#CC#CCOCCOCCN*] | (264) |
| 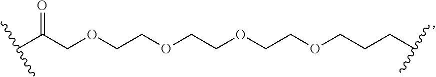 [SMILES: *CCCOCCOCCOCCOCC(*)=O] | (265) |
| 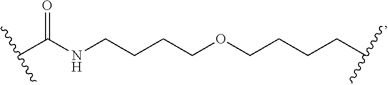 [SMILES: *CCCCOCCCCNC(*)=O] | (266) |
| 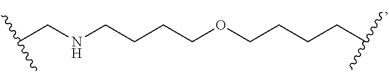 [SMILES: *CCCCOCCCCNC*] | (267) |
| 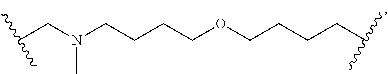 [SMILES: *CCCCOCCCCN(C)C*] | (268) |
| 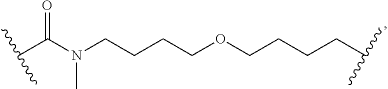 [SMILES: *CCCCOCCCCN(C)C(*)=O] | (269) |
| 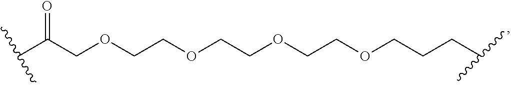 [SMILES: *CCCOCCOCCOCCOCC(*)=O] | (270) |
| 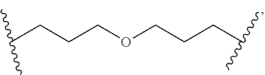 [SMILES: *CCCOCCC*] | (271) |
| 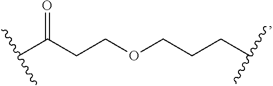 [SMILES: *CCCOCCC(*)=O] | (272) |
| 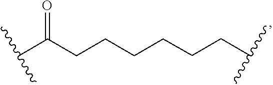 [SMILES: *CCCCCCC(*)=O] | (273) |
| 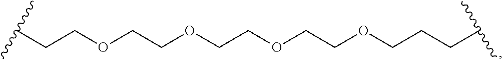 [SMILES: *CCCOCCOCCOCCOCC*] | (274) |
| 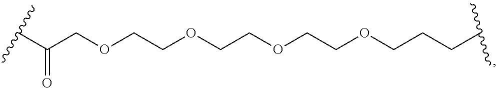 [SMILES: *CCCOCCOCCOCCOCC(*)=O] | (275) |
| 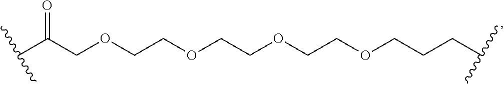 [SMILES: *CCCOCCOCCOCCOCC(*)=O] | (276) |
| 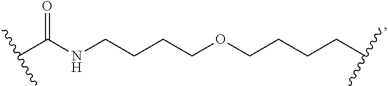 [SMILES: *CCCCOCCCCNC(*)=O] | (277) |
| 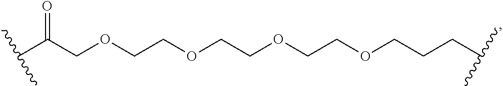 [SMILES: *CCCOCCOCCOCCOCC(*)=O] | (278) |
| 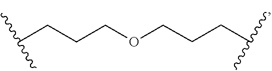 [SMILES: *CCCOCCC*] | (279) |
| 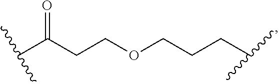 [SMILES: *CCCOCCC(*)=O] | (280) |
| 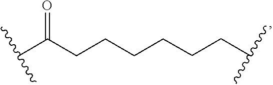 [SMILES: *CCCCCCC(*)=O] | (281) |
| 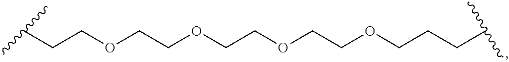 [SMILES: *CCCOCCOCCOCCOCC*] | (282) |
| 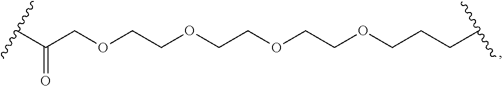 [SMILES: *CCCOCCOCCOCCOCC(*)=O] | (283) |
| 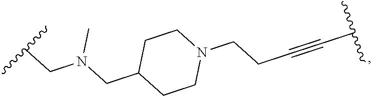 [SMILES: *C#CCCN1CCC(CN(C)C*)CC1] | (284) |
| 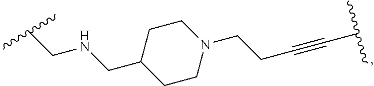 [SMILES: *C#CCCN1CCC(CNC*)CC1] | (285) |
| 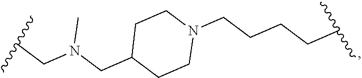 [SMILES: *CCCCN1CCC(CN(C)C*)CC1] | (286) |
| 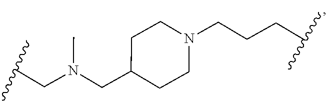 [SMILES: *CCCN1CCC(CN(C)C*)CC1] | (287) |
| 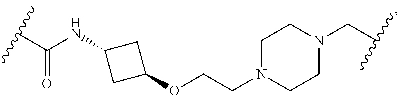 [SMILES: *CN1CCN(CCO[C@H]2C[C@H](NC(*)=O)C2)CC1] | (288) |
| 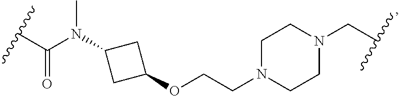 [SMILES: *CN1CCN(CCO[C@H]2C[C@H](N(C)C(*)=O)C2)CC1] | (289) |
| 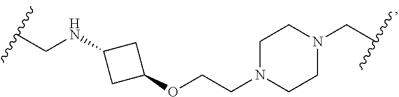 [SMILES: *CN[C@H]1C[C@H](OCCN2CCN(C*)CC2)C1] | (290) |
| 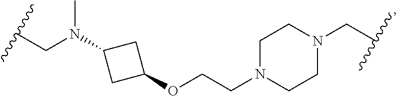 [SMILES: *CN1CCN(CCO[C@H]2C[C@H](N(C)C*)C2)CC1] | (291) |
| 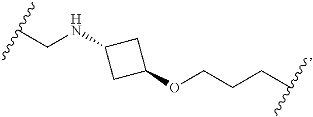 [SMILES: *CCCO[C@H]1C[C@H](NC*)C1] | (292) |
| 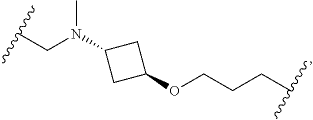 [SMILES: *CCCO[C@H]1C[C@H](N(C)C*)C1] | (293) |
| 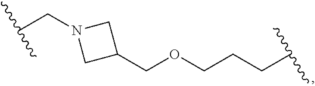 [SMILES: *CCCOCC1CN(C*)C1] | (294) |
| 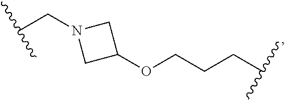 [SMILES: *CCCOC1CN(C*)C1] | (295) |
| 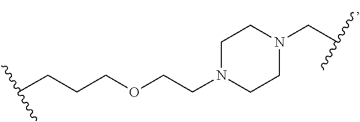 [SMILES: *CCCOCCN1CCN(C*)CC1] | (296) |
| 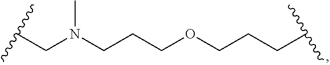 [SMILES: *CCCOCCCN(C)C*] | (297) |
| 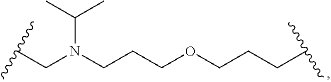 [SMILES: *CCCOCCCN(C*)C(C)C] | (298) |
| 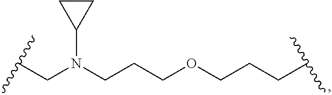 [SMILES: *CCCOCCCN(C*)C1CC1] | (299) |
| 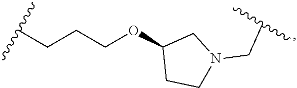 [SMILES: *CCCO[C@@H]1CCN(C*)C1] | (300) |
| 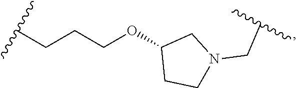 [SMILES: *CCCO[C@H]1CCN(C*)C1] | (301) |
| 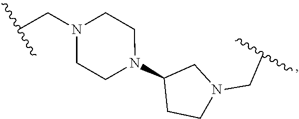 [SMILES: *CN1CCN([C@@H]2CCN(C*)C2)CC1] | (302) |
| 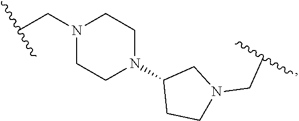 [SMILES: *CN1CCN([C@H]2CCN(C*)C2)CC1] | (303) |
| 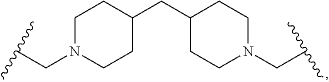 [SMILES: *CN1CCC(CC2CCN(C*)CC2)CC1] | (304) |
| 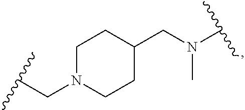 [SMILES: *CN1CCC(CN(*)C)CC1] | (305) |
| 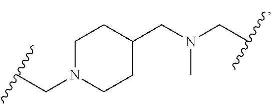 [SMILES: *CN(C)CC1CCN(C*)CC1] | (306) |
| 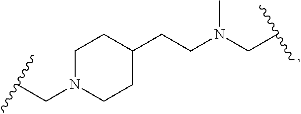 [SMILES: *CN(C)CCC1CCN(C*)CC1] | (307) |
| 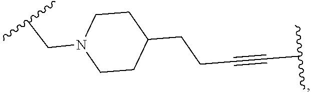 [SMILES: *C#CCCC1CCN(C*)CC1] | (308) |
| 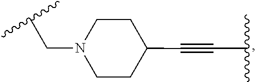 [SMILES: *C#CC1CCN(C*)CC1] | (309) |
| 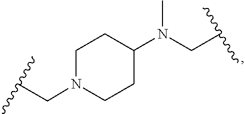 [SMILES: *CN1CCC(N(C)C*)CC1] | (310) |
| 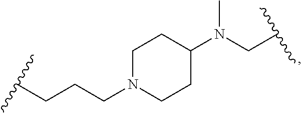 [SMILES: *CCCN1CCC(N(C)C*)CC1] | (311) |
| 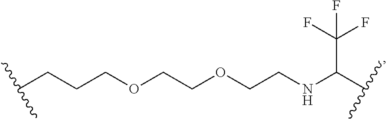 [SMILES: *CCCOCCOCCNC(*)C(F)(F)F] | (312) |
| 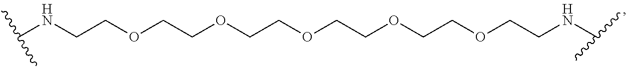 [SMILES: *NCCOCCOCCOCCOCCOCCN*] | (313) |
| 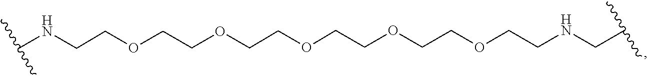 [SMILES: *CNCCOCCOCCOCCOCCOCCN*] | (314) |
| 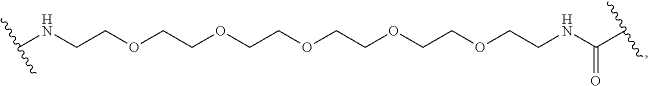 [SMILES: *NCCOCCOCCOCCOCCOCCNC(*)=O] | (315) |
| 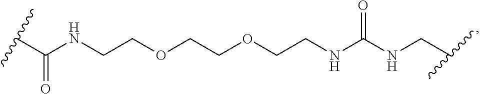 [SMILES: *CNC(=O)NCCOCCOCCNC(*)=O] | (316) |
| 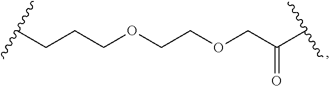 [SMILES: *CCCOCCOCC(*)=O] | (317) |
| 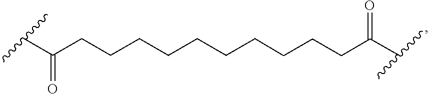 [SMILES: *C(=O)CCCCCCCCCCC(*)=O] | (318) |
| 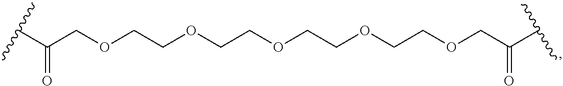 [SMILES: *C(=O)COCCOCCOCCOCCOCC(*)=O] | (319) |
| 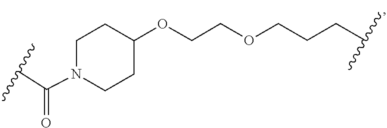 [SMILES: *CCCOCCOC1CCN(C(*)=O)CC1] | (320) |
| 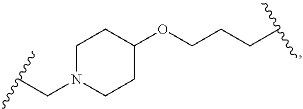 [SMILES: *CCCOC1CCN(C*)CC1] | (321) |
| 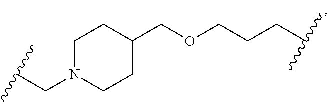 [SMILES: *CCCOCC1CCN(C*)CC1] | (322) |
| 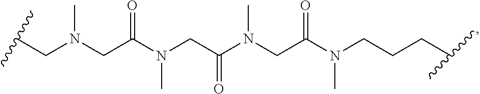 [SMILES: *CCCN(C)C(=O)CN(C)C(=O)CN(C)C(=O)CN(C)C*] | (323) |
| 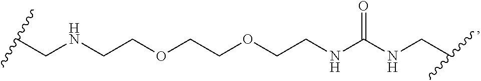 [SMILES: *CNCCOCCOCCNC(=O)NC*] | (324) |
| 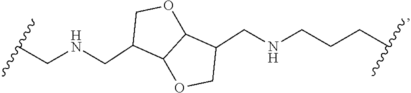 [SMILES: *CCCNCC1COC2C(CNC*)COC12] | (325) |
| 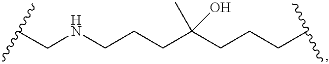 [SMILES: *CCCC(C)(O)CCCNC*] | (326) |
| 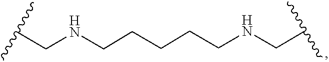 [SMILES: *CNCCCCCNC*] | (327) |
| 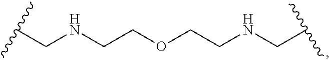 [SMILES: *CNCCOCCNC*] | (328) |
| 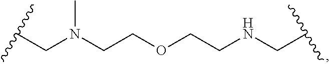 [SMILES: *CNCCOCCN(C)C*] | (329) |
| 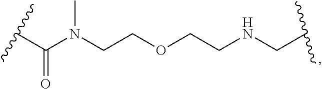 [SMILES: *CNCCOCCN(C)C(*)=O] | (330) |
| 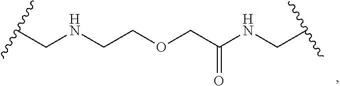 [SMILES: *CNCCOCC(=O)NC*] | (331) |
| 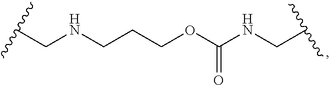 [SMILES: *CNCCCOC(=O)NC*] | (332) |
| 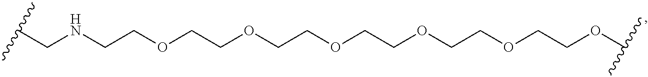 [SMILES: *CNCCOCCOCCOCCOCCOCCO*] | (333) |
| 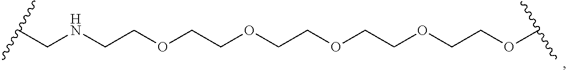 [SMILES: *CNCCOCCOCCOCCOCCO*] | (334) |
| 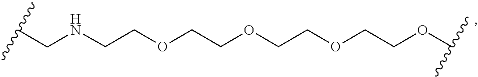 [SMILES: *CNCCOCCOCCOCCO*] | (335) |
| 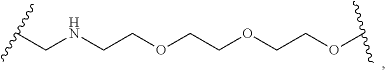 [SMILES: *CNCCOCCOCCO*] | (336) |
| 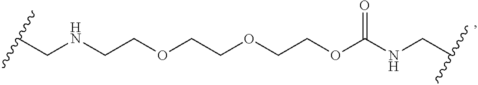 [SMILES: *CNCCOCCOCCOC(=O)NC*] | (337) |
| 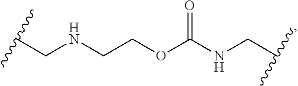 [SMILES: *CNCCOC(=O)NC*] | (338) |
| 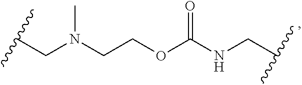 [SMILES: *CNC(=O)OCCN(C)C*] | (339) |
| 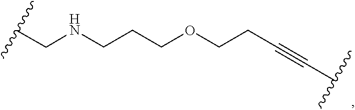 [SMILES: *C#CCCOCCCNC*] | (340) |
| 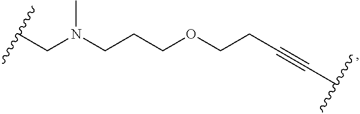 [SMILES: *C#CCCOCCCN(C)C*] | (341) |
| 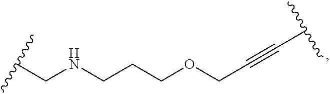 [SMILES: *C#CCOCCCNC*] | (342) |
| 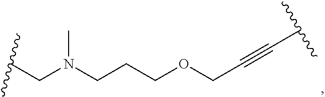 [SMILES: *C#CCOCCCN(C)C*] | (343) |
| 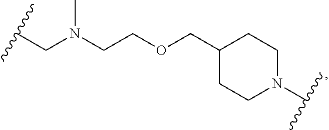 [SMILES: *CN(C)CCOCC1CCN(*)CC1] | (344) |
| 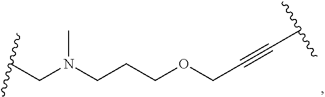 [SMILES: *C#CCOCCCN(C)C*] | (343) |
| 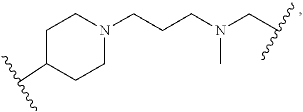 [SMILES: *CN(C)CCCN1CCC(*)CC1] | (346) |
| 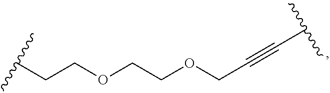 [SMILES: *C#CCOCCOCC*] | (347) |
| 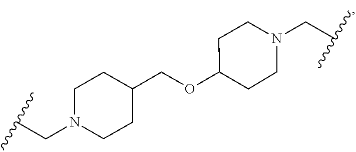 [SMILES: *CN1CCC(COC2CCN(C*)CC2)CC1] | (348) |
| 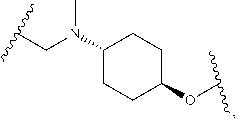 [SMILES: *CN(C)[C@H]1CC[C@H](O*)CC1] | (349) |
| 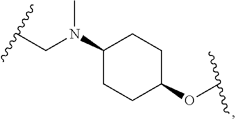 [SMILES: *CN(C)[C@H]1CC[C@@H](O*)CC1] | (350) |
| 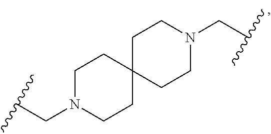 [SMILES: *CN1CCC2(CC1)CCN(C*)CC2] | (351) |
| 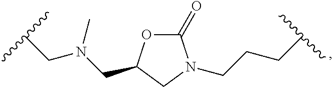 [SMILES: *CCCN1C[C@@H](CN(C)C*)OC1=O] | (352) |
| 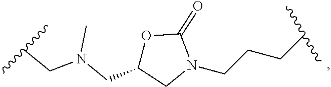 [SMILES: *CCCN1C[C@H](CN(C)C*)OC1=O] | (353) |
| 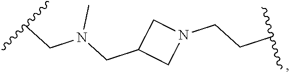 [SMILES: *CCN1CC(CN(C)C*)C1] | (354) |
| 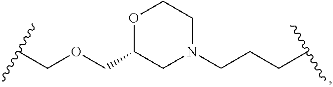 [SMILES: *CCCN1CCO[C@@H](COC*)C1] | (355) |
| 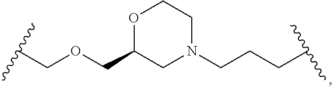 [SMILES: *CCCN1CCO[C@H](COC*)C1] | (356) |
| 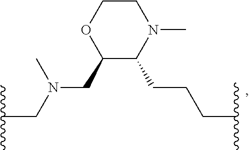 [SMILES: *CCC[C@@H]1[C@@H](CN(C)C*)OCCN1C] | (357) |
| 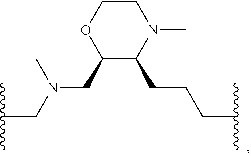 [SMILES: *CCC[C@H]1[C@@H](CN(C)C*)OCCN1C] | (358) |
| 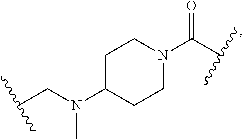 [SMILES: *CN(C)C1CCN(C(*)=O)CC1] | (359) |
| 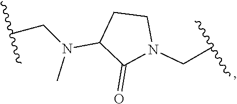 [SMILES: *CN1CCC(N(C)C*)C1=O] | (360) |
| 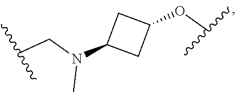 [SMILES: *CN(C)[C@H]1C[C@H](O*)C1] | (361) |
| 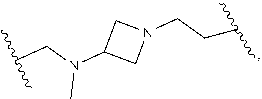 [SMILES: *CCN1CC(N(C)C*)C1] | (362) |
| 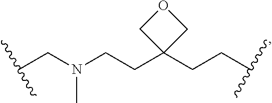 [SMILES: *CCC1(CCN(C)C*)COC1] | (363) |
| 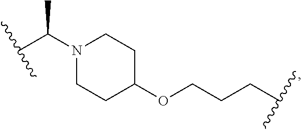 [SMILES: *CCCOC1CCN([C@@H](*)C)CC1] | (364) |
| 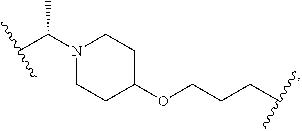 [SMILES: *CCCOC1CCN([C@H](*)C)CC1] | (365) |
| 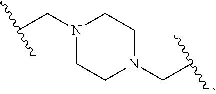 [SMILES: *CN1CCN(C*)CC1] | (366) |
| 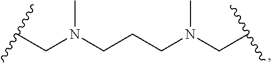 [SMILES: *CN(C)CCCN(C)C*] | (367) |
| 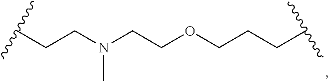 [SMILES: *CCCOCCN(C)CC*] | (368) |
| 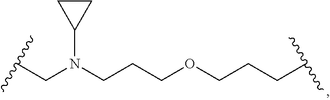 [SMILES: *CCCOCCCN(C*)C1CC1] | (369) |
| 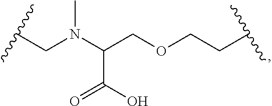 [SMILES: *CCOCC(C(=O)O)N(C)C*] | (370) |
| 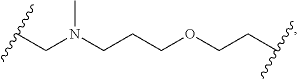 [SMILES: *CCOCCCN(C)C*] | (371) |
| 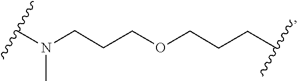 | (372) |
| 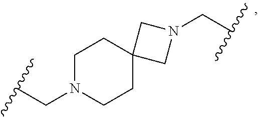 [SMILES: *CN1CCC2(CC1)CN(C*)C2] | (373) |
| 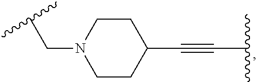 [SMILES: *C#CC1CCN(C*)CC1] | (374) |
| 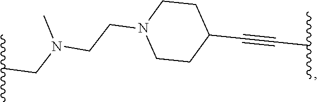 [SMILES: *C#CC1CCN(CCN(C)C*)CC1] | (375) |
| 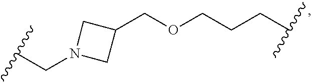 [SMILES: *CCCOCC1CN(C*)C1] | (376) |
| 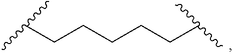 [SMILES: *CCCCC*] | (377) |
| 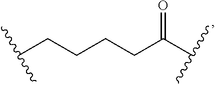 [SMILES: *CCCCC(*)=O] | (378) |
| 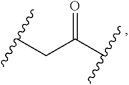 [SMILES: *CC(*)=O] | (379) |
| 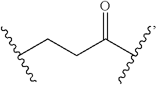 [SMILES: *CCC(*)=O] | (380) |
| 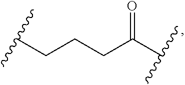 [SMILES: *CCCC(*)=O] | (381) |
| 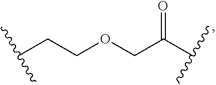 [SMILES: *CCOCC(*)=O] | (382) |
| 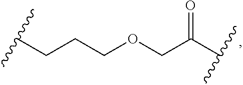 [SMILES: *CCCOCC(*)=O] | (383) |
| 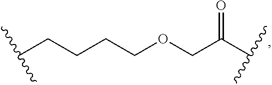 [SMILES: *CCCCOCC(*)=O] | (384) |
| 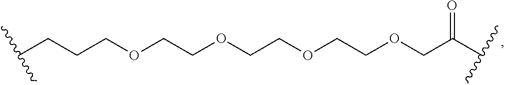 [SMILES: *CCCOCCOCCOCCOCC(*)=O] | (385) |
| 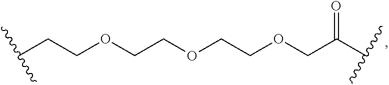 [SMILES: *CCOCCOCCOCC(*)=O] | (386) |
| 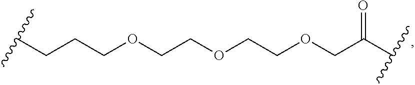 [SMILES: *CCCOCCOCCOCC(*)=O] | (387) |
| 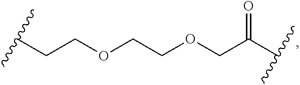 [SMILES: *CCOCCOCC(*)=O] | (388) |
| 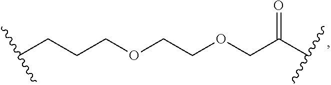 [SMILES: *CCCOCCOCC(*)=O] | (389) |
| 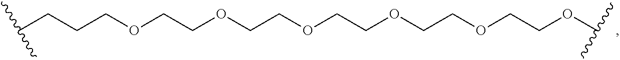 [SMILES: *CCCOCCOCCOCCOCCOCCO*] | (390) |
| 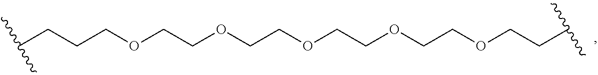 [SMILES: *CCCOCCOCCOCCOCCOCC*] | (391) |
| 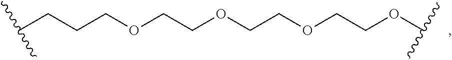 [SMILES: *CCCOCCOCCOCCO*] | (392) |
| 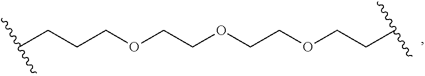 [SMILES: *CCCOCCOCCOCC*] | (393) |
| 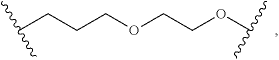 [SMILES: *CCCOCCO*] | (394) |
| 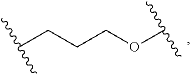 [SMILES: *CCCO*] | (395) |
| 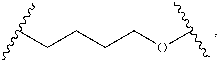 [SMILES: *CCCCO*] | (396) |
| 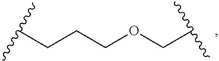 [SMILES: *CCCOC*] | (397) |
| 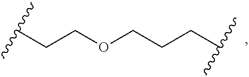 [SMILES: *CCCOCC*] | (398) |
| 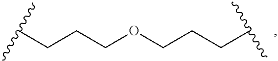 [SMILES: *CCCOCCC*] | (399) |
| 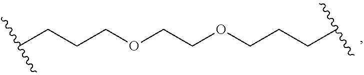 [SMILES: *CCCOCCOCCC*] | (400) |
| 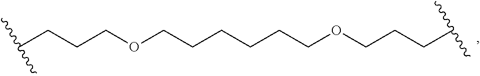 [SMILES: *CCCOCCCCCCOCCC*] | (401) |
| 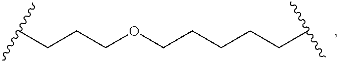 [SMILES: *CCCCCOCCC*] | (402) |
| 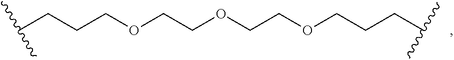 [SMILES: *CCCOCCOCCOCCC*] | (403) |
| 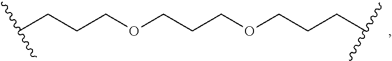 [SMILES: *CCCOCCCOCCC*] | (404) |
| 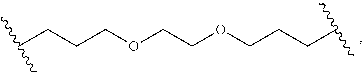 [SMILES: *CCCOCCOCCC*] | (405) |
| 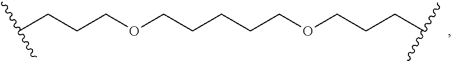 [SMILES: *CCCOCCCCCOCCC*] | (406) |
| 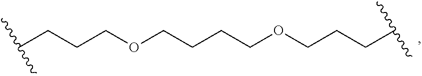 [SMILES: *CCCOCCCCOCCC*] | (407) |
| 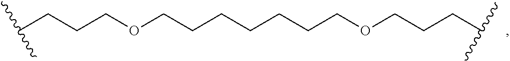 [SMILES: *CCCOCCCCCCCOCCC*] | (408) |
| 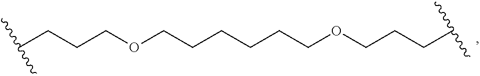 [SMILES: *CCCOCCCCCCOCCC*] | (409) |
| 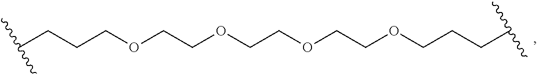 [SMILES: *CCCOCCOCCOCCOCCC*] | (410) |
| 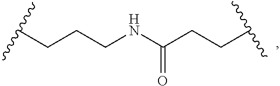 [SMILES: *CCCNC(=O)CC*] | (411) |
| 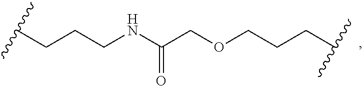 [SMILES: *CCCNC(=O)COCCC*] | (412) |
| 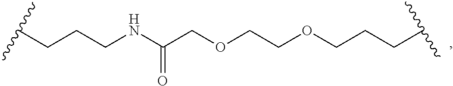 [SMILES: *CCCNC(=O)COCCOCCC*] | (413) |
| 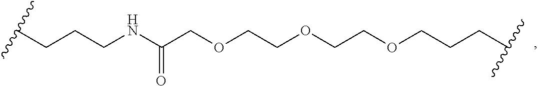 [SMILES: *CCCNC(=O)COCCOCCOCCC*] | (414) |
| 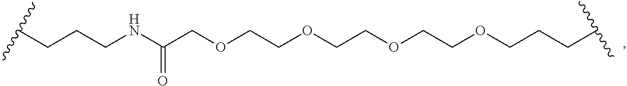 [SMILES: *CCCNC(=O)COCCOCCOCCOCCC*] | (415) |
| 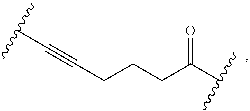 [SMILES: *C#CCCCC(*)=O] | (416) |
| 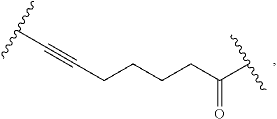 [SMILES: *C#CCCCCC(*)=O] | (417) |
| 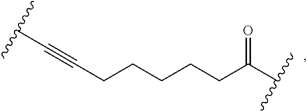 [SMILES: *C#CCCCCCC(*)=O] | (418) |
| 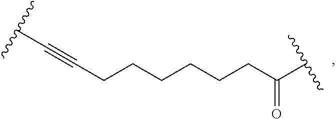 [SMILES: *C#CCCCCCCC(*)=O] | (419) |
| 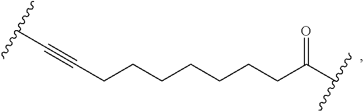 [SMILES: *C#CCCCCCCCC(*)=O] | (420) |
| 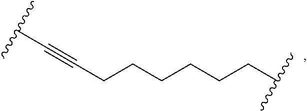 [SMILES: *C#CCCCCCC*] | (421) |
| 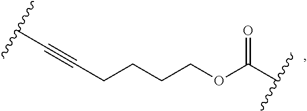 [SMILES: *C#CCCCCOC(*)=O] | (422) |
| 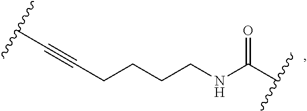 [SMILES: *C#CCCCCNC(*)=O] | (423) |
| 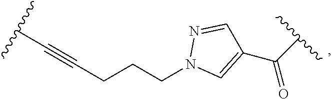 [SMILES: *C#CCCCn1cc(C(*)=O)cn1] | (424) |
| 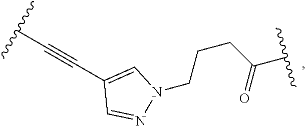 [SMILES: *C#Cc1cnn(CCCC(*)=O)c1] | (425) |
| 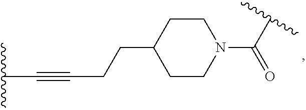 [SMILES: *C#CCCC1CCN(C(*)=O)CC1] | (426) |
| 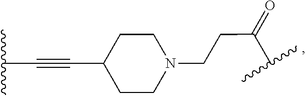 [SMILES: *C#CC1CCN(CCC(*)=O)CC1] | (427) |
| 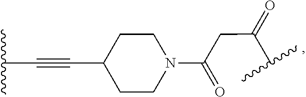 [SMILES: *C#CC1CCN(C(=O)CC(*)=O)CC1] | (428) |
| 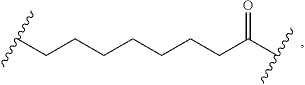 [SMILES: *CCCCCCCC(*)=O] | (429) |
| 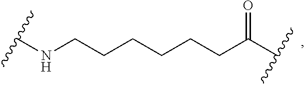 [SMILES: *NCCCCCCC(*)=O] | (430) |
| 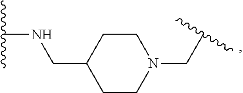 [SMILES: *CN1CCC(CN*)CC1] | (431) |
| 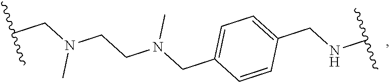 [SMILES: *CN(C)CCN(C)Cc1ccc(CN*)cc1] | (432) |
| 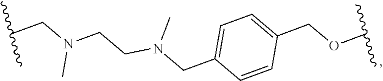 [SMILES: *CN(C)CCN(C)Cc1ccc(CO*)cc1] | (433) |
| 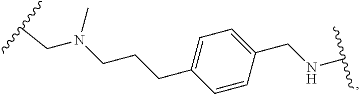 [SMILES: *CN(C)CCCc1ccc(CN*)cc1] | (434) |
| 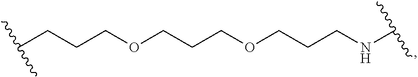 [SMILES: *CCCOCCCOCCCN*] | (435) |
| 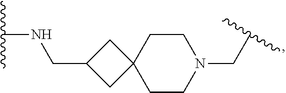 [SMILES: *CN1CCC2(CC1)CC(CN*)C2] | (436) |
| 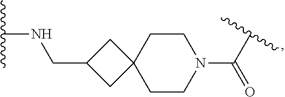 [SMILES: *NCC1CC2(CCN(C(*)=O)CC2)C1] | (437) |
| 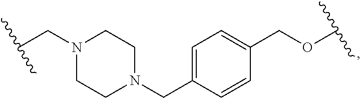 [SMILES: *CN1CCN(Cc2ccc(CO*)cc2)CC1] | (438) |
| 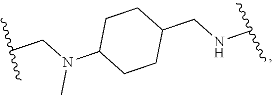 [SMILES: *CN(C)C1CCC(CN*)CC1] | (438) |
| 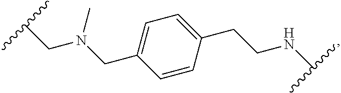 [SMILES: *CN(C)Cc1ccc(CCN*)cc1] | (439) |
| 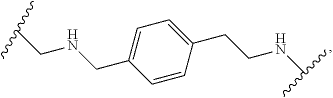 [SMILES: *CNCc1ccc(CCN*)cc1] | (440) |
| 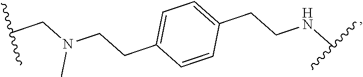 [SMILES: *CN(C)CCc1ccc(CCN*)cc1] | (441) |
| 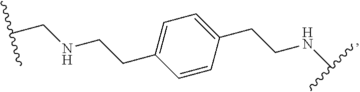 [SMILES: *CNCCc1ccc(CCN*)cc1] | (442) |
| 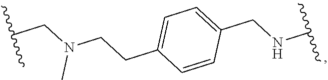 [SMILES: *CN(C)CCc1ccc(CN*)cc1] | (443) |
| 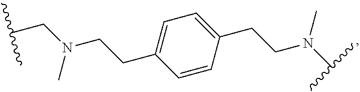 [SMILES: *CN(C)CCc1ccc(CCN(*)C)cc1] | (444) |
| 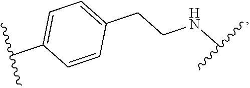 [SMILES: *NCCc1ccc(*)cc1] | (445) |
| 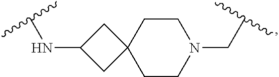 [SMILES: *CN1CCC2(CC1)CC(N*)C2] | (446) |
| 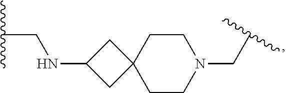 [SMILES: *CNC1CC2(CCN(C*)CC2)C1] | (447) |
| 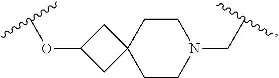 [SMILES: *CN1CCC2(CC1)CC(O*)C2] | (448) |
| 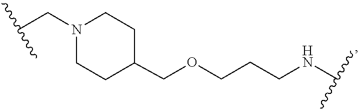 [SMILES: *CN1CCC(COCCCN*)CC1] | (449) |
| 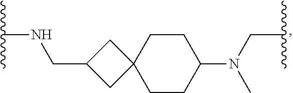 [SMILES: *CN(C)C1CCC2(CC1)CC(CN*)C2] | (450) |
| 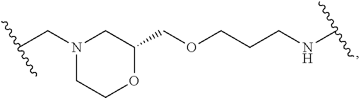 [SMILES: *CN1CCO[C@@H](COCCCN*)C1] | (451) |
| 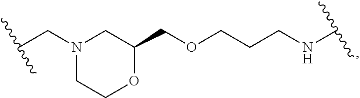 [SMILES: *CN1CCO[C@H](COCCCN*)C1] | (452) |
| 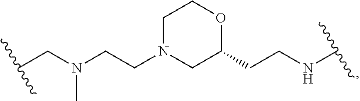 [SMILES: *CN(C)CCN1CCO[C@H](CCN*)C1] | (453) |
| 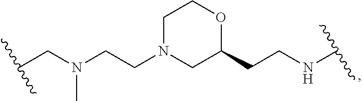 [SMILES: *CN(C)CCN1CCO[C@@H](CCN*)C1] | (454) |
| 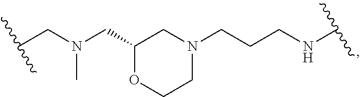 [SMILES: *CN(C)C[C@@H]1CN(CCCN*)CCO1] | (455) |
| 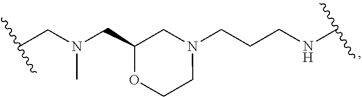 [SMILES: *CN(C)C[C@H]1CN(CCCN*)CCO1] | (456) |
| 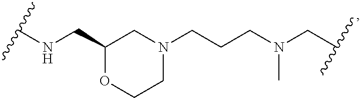 [SMILES: *CN(C)CCCN1CCO[C@@H](CN*)C1] | (457) |
| 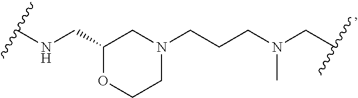 [SMILES: *CN(C)CCCN1CCO[C@H](CN*)C1] | (458) |
| 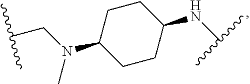 [SMILES: *CN(C)[C@H]1CC[C@@H](N*)CC1] | (459) |
| 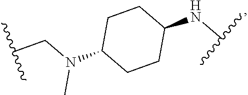 [SMILES: *CN(C)[C@H]1CC[C@H](N*)CC1] | (460) |
| 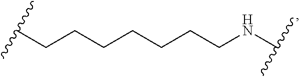 [SMILES: *CCCCCCCN*] | (461) |
| 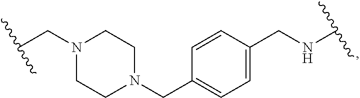 [SMILES: *CN1CCN(Cc2ccc(CN*)cc2)CC1] | (462) |
| 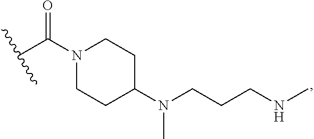 [SMILES: *C(=O)N1CCC(N(C)CCCNC)CC1] | (463) |
| 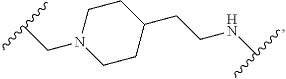 [SMILES: *CN1CCC(CCN*)CC1] | (464) |
| 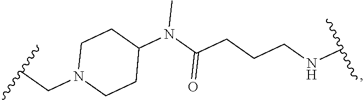 [SMILES: *CN1CCC(N(C)C(=O)CCCN*)CC1] | (465) |
| 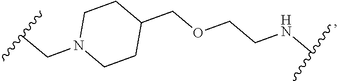 [SMILES: *CN1CCC(COCCN*)CC1] | (466) |
| 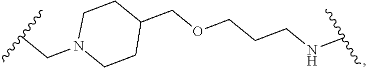 [SMILES: *CN1CCC(COCCCN*)CC1] | (467) |
| 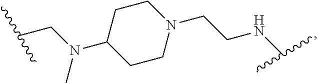 [SMILES: *CN(C)C1CCN(CCN*)CC1] | (468) |
| 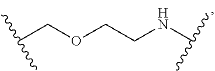 [SMILES: *COCCN*] | (469) |
| 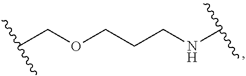 [SMILES: *COCCCN*] | (470) |
| 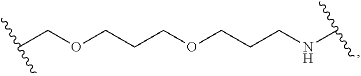 [SMILES: *COCCCOCCCN*] | (471) |
| 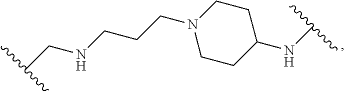 [SMILES: *CNCCCN1CCC(N*)CC1] | (472) |
| 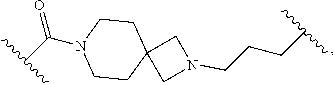 [SMILES: *CCCN1CC2(CCN(C(*)=O)CC2)C1] | (473) |
| 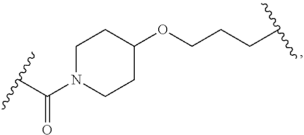 [SMILES: *CCCOC1CCN(C(*)=O)CC1] | (474) |
| 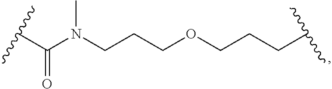 [SMILES: *CCCOCCCN(C)C(*)=O] | (475) |
| 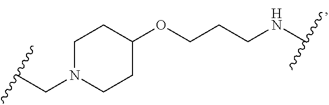 [SMILES: *CN1CCC(OCCCN*)CC1] | (475) |
| 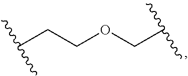 [SMILES: *CCOC*] | (476) |
| 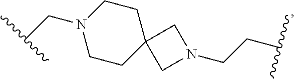 [SMILES: *CCN1CC2(CCN(C*)CC2)C1] | (477) |
| 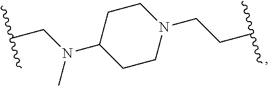 [SMILES: *CCN1CCC(N(C)C*)CC1] | (478) |
| 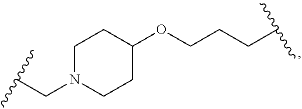 [SMILES: *CCCOC1CCN(C*)CC1] | (479) |
| 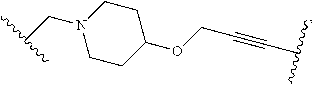 [SMILES: *C#CCOC1CCN(C*)CC1] | (480) |
| 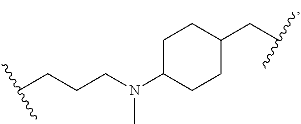 [SMILES: *CCCN(C)C1CCC(C*)CC1] | (481) |
| 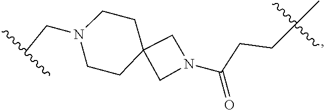 [SMILES: *CCC(=O)N1CC2(CCN(C*)CC2)C1] | (482) |
| 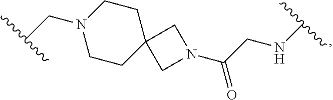 [SMILES: *CN1CCC2(CC1)CN(C(=O)CN*)C2] | (483) |
| 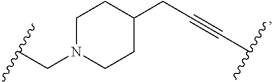 [SMILES: *C#CCC1CCN(C*)CC1] | (484) |
| 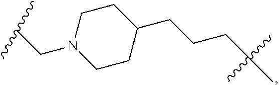 [SMILES: *CCCC1CCN(C*)CC1] | (485) |
| 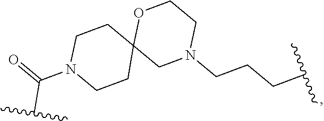 [SMILES: *CCCN1CCOC2(CCN(C(*)=O)CC2)C1] | (486) |
| 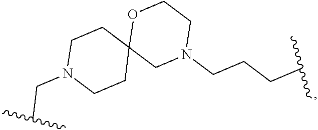 [SMILES: *CCCN1CCOC2(CCN(C*)CC2)C1] | (487) |
| 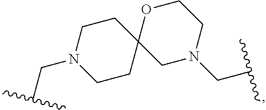 [SMILES: *CN1CCC2(CC1)CN(C*)CCO2] | (488) |
| 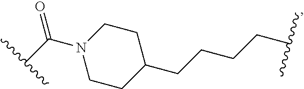 [SMILES: *CCCCC1CCN(C(*)=O)CC1] | (489) |
| 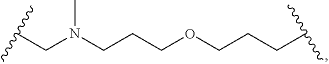 [SMILES: *CCCOCCCN(C)C*] | (490) |
| 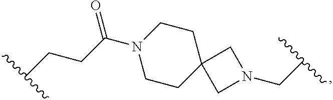 [SMILES: *CCC(=O)N1CCC2(CC1)CN(C*)C2] | (491) |
| 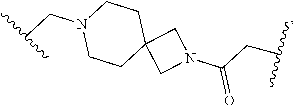 [SMILES: *CC(=O)N1CC2(CCN(C*)CC2)C1] | (492) |
| 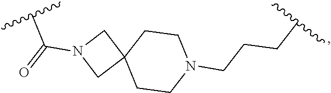 [SMILES: *CCCN1CCC2(CC1)CN(C(*)=O)C2] | (493) |
| 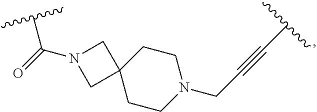 [SMILES: *C#CCN1CCC2(CC1)CN(C(*)=O)C2] | (494) |
| 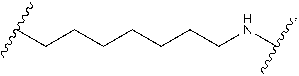 [SMILES: *CCCCCCCN*] | (495) |
| 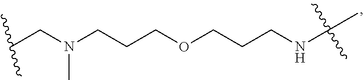 [SMILES: *CN(C)CCCOCCCN*] | (496) |
| 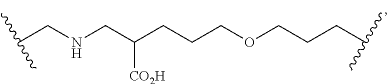 [SMILES: *CCCOCCCC(CNC*)C(=O)O] | (497) |
| 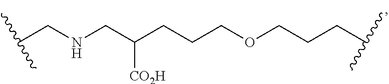 [SMILES: *CCCOCCCC(CNC*)C(=O)O] | (498) |
| 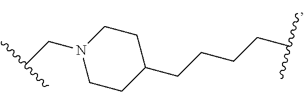 [SMILES: *CCCCC1CCN(C*)CC1] | (499) |
| 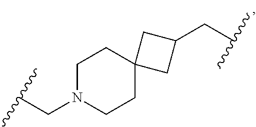 [SMILES: *CC1CC2(CCN(C*)CC2)C1] | (500) |
| 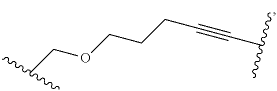 [SMILES: *C#CCCCOC*] | (501) |
| 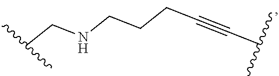 [SMILES: *C#CCCCNC*] | (502) |
| 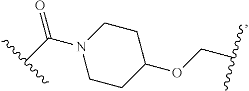 [SMILES: *COC1CCN(C(*)=O)CC1] | (503) |
| 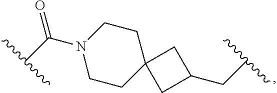 [SMILES: *CC1CC2(CCN(C(*)=O)CC2)C1] | (504) |
| 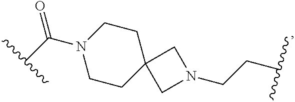 [SMILES: *CCN1CC2(CCN(C(*)=O)CC2)C1] | (505) |
| 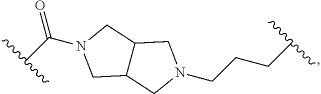 [SMILES: *CCCN1CC2CN(C(*)=O)CC2C1] | (506) |
| 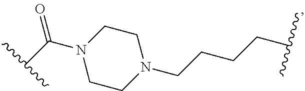 [SMILES: *CCCCN1CCN(C(*)=O)CC1] | (507) |
| 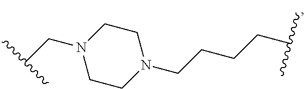 [SMILES: *CCCCN1CCN(C*)CC1] | (508) |
| 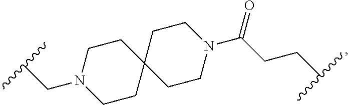 [SMILES: *CCC(=O)N1CCC2(CCN(C*)CC2)CC1] | (509) |
| 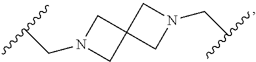 [SMILES: *CN1CC2(C1)CN(C*)C2] | (510) |
| 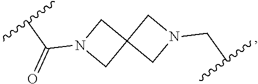 [SMILES: *CN1CC2(C1)CN(C(*)=O)C2] | (511) |
| 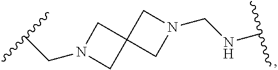 [SMILES: *CN1CC2(C1)CN(CN*)C2] | (512) |
| 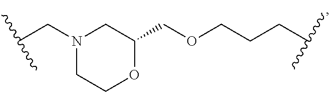 [SMILES: *CCCOC[C@H]1CN(C*)CCO1] | (513) |
| 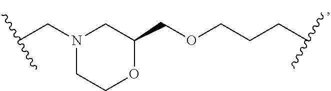 [SMILES: *CCCOC[C@@H]1CN(C*)CCO1] | (514) |
| 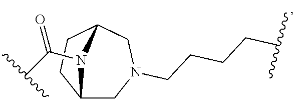 [SMILES: *CCCCN1C[C@H]2CC[C@@H](C1)N2C(*)=O] | (515) |
| 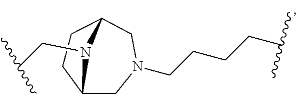 [SMILES: *CCCCN1C[C@H]2CC[C@@H](C1)N2C*] | (516) |
| 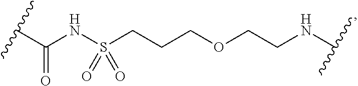 [SMILES: *NCCOCCCS(=O)(=O)NC(*)=O] | (517) |
| 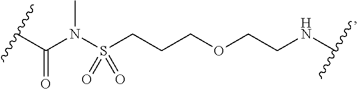 [SMILES: *NCCOCCCS(=O)(=O)N(C)C(*)=O] | (518) |
| 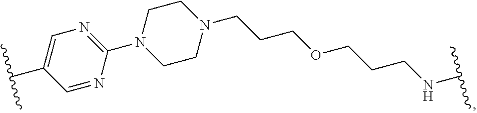 [SMILES: *NCCCOCCCN1CCN(c2ncc(*)cn2)CC1] | (519) |
| 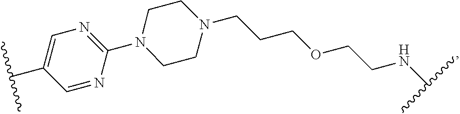 [SMILES: *NCCOCCCN1CCN(c2ncc(*)cn2)CC1] | (520) |
| 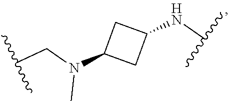 [SMILES: *CN(C)[C@H]1C[C@H](N*)C1] | (521) |
| 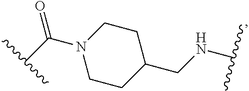 [SMILES: *NCC1CCN(C(*)=O)CC1] | (522) |
| 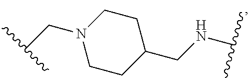 [SMILES: *CN1CCC(CN*)CC1] | (523) |
| 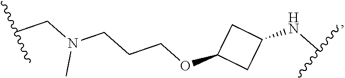 [SMILES: *CN(C)CCCO[C@H]1C[C@H](N*)C1] | (524) |
| 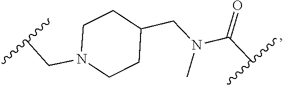 [SMILES: *CN1CCC(CN(C)C(*)=O)CC1] | (525) |
| 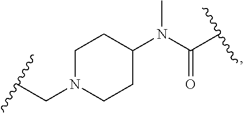 [SMILES: *CN1CCC(N(C)C(*)=O)CC1] | (526) |
| 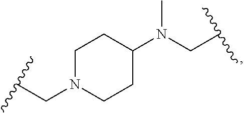 [SMILES: *CN1CCC(N(C)C*)CC1] | (527) |
| 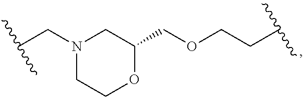 [SMILES: *CCOC[C@H]1CN(C*)CCO1] | (528) |
| 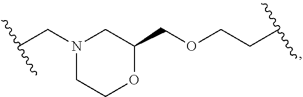 [SMILES: *CCOC[C@@H]1CN(C*)CCO1] | (529) |
| 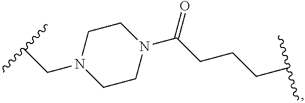 [SMILES: *CCCC(=O)N1CCN(C*)CC1] | (530) |
| 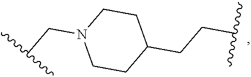 [SMILES: *CCC1CCN(C*)CC1] | (531) |
| 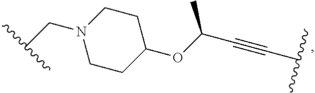 [SMILES: *C#C[C@H](C)OC1CCN(C*)CC1] | (532) |
| 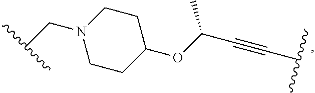 [SMILES: *C#C[C@@H](C)OC1CCN(C*)CC1] | (533) |
| 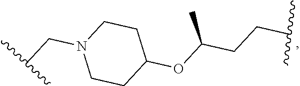 [SMILES: *CC[C@H](C)OC1CCN(C*)CC1] | (534) |
| 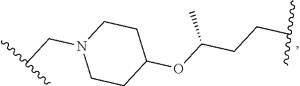 [SMILES: *CC[C@@H](C)OC1CCN(C*)CC1] | (535) |
| 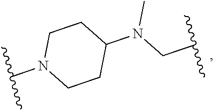 [SMILES: *CN(C)C1CCN(*)CC1] | (536) |
| 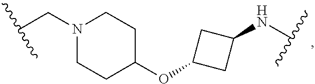 [SMILES: *CN1CCC(O[C@H]2C[C@H](N*)C2)CC1] | (537) |
| 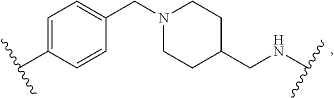 [SMILES: *NCC1CCN(Cc2ccc(*)cc2)CC1] | (538) |
| 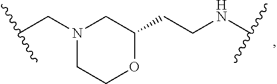 [SMILES: *CN1CCO[C@@H](CCN*)C1] | (539) |
| 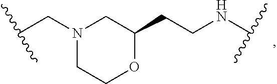 [SMILES: *CN1CCO[C@H](CCN*)C1] | (540) |
| 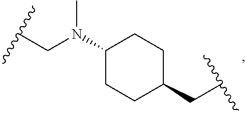 [SMILES: *CN(C)[C@H]1CC[C@H](C*)CC1] | (541) |
| 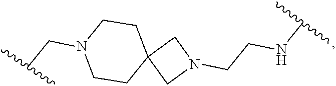 [SMILES: *CN1CCC2(CC1)CN(CCN*)C2] | (542) |
| 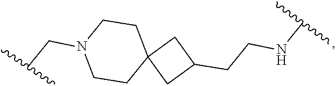 [SMILES: *CN1CCC2(CC1)CC(CCN*)C2] | (543) |
| 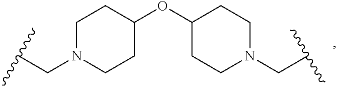 [SMILES: *CN1CCC(OC2CCN(C*)CC2)CC1] | (544) |
| 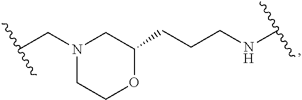 [SMILES: *CN1CCO[C@@H](CCCN*)C1] | (545) |
| 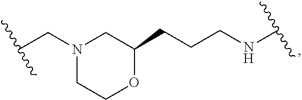 [SMILES: *CN1CCO[C@H](CCCN*)C1] | (546) |
| 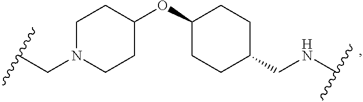 [SMILES: *CN1CCC(O[C@H]2CC[C@H](CN*)CC2)CC1] | (547) |
| 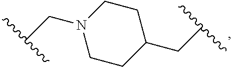 [SMILES: *CC1CCN(C*)CC1] | (548) |
| 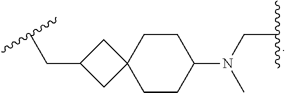 [SMILES: *CC1CC2(CCC(N(C)C*)CC2)C1] | (549) |
| 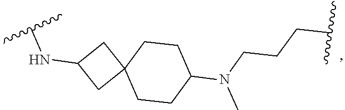 [SMILES: *CCCN(C)C1CCC2(CC1)CC(N*)C2] | (550) |
| 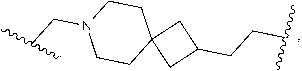 [SMILES: *CCC1CC2(CCN(C*)CC2)C1] | (551) |
| 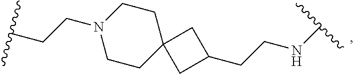 [SMILES: *CCN1CCC2(CC1)CC(CCN*)C2] | (552) |
| 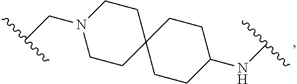 [SMILES: *CN1CCC2(CCC(N*)CC2)CC1] | (553) |
| 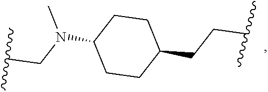 [SMILES: *CC[C@H]1CC[C@H](N(C)C*)CC1] | (554) |
| 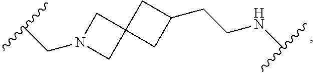 [SMILES: *CN1CC2(CC(CCN*)C2)C1] | (555) |
| 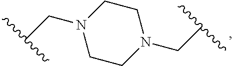 [SMILES: *CN1CCN(C*)CC1] | (556) |
| 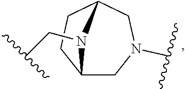 [SMILES: *CN1[C@@H]2CC[C@H]1CN(*)C2] | (557) |
| 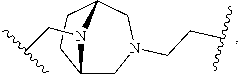 [SMILES: *CCN1C[C@H]2CC[C@@H](C1)N2C*] | (558) |
| 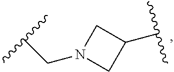 [SMILES: *CN1CC(*)C1] | (559) |
| 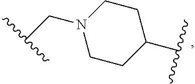 [SMILES: *CN1CCC(*)CC1] | (560) |
| 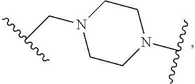 [SMILES: *CN1CCN(*)CC1] | (561) |
| 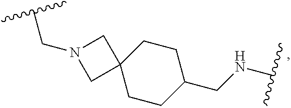 [SMILES: *CN1CC2(CCC(CN*)CC2)C1] | (562) |
| 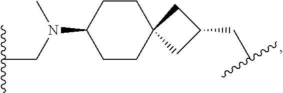 | (563) |
| 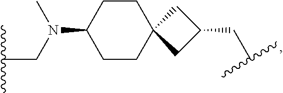 | (564) |
| 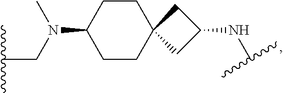 | (565) |
| 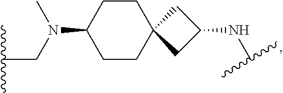 | (566) |
| 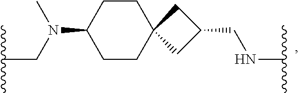 | (567) |
| 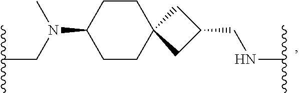 | (568) |
| 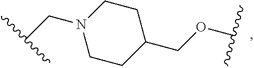 [SMILES: *CN1CCC(CO*)CC1] | (569) |
| 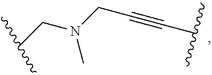 [SMILES: *C#CCN(C)C*] | (570) |
| 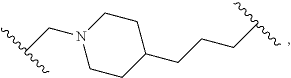 [SMILES: *CCCC1CCN(C*)CC1] | (571) |
| 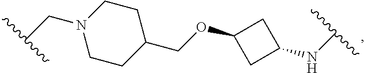 [SMILES: *CN1CCC(CO[C@H]2C[C@H](N*)C2)CC1] | (572) |
| 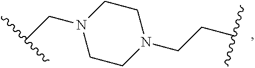 [SMILES: *CCN1CCN(C*)CC1] | (573) |
| 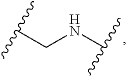 [SMILES: *CN*] | (574) |
| 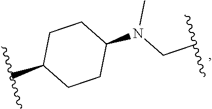 [SMILES: *CN(C)[C@H]1CC[C@@H](*)CC1] | (575) |
| 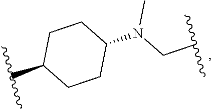 [SMILES: *CN(C)[C@H]1CC[C@H](*)CC1] | (576) |
| 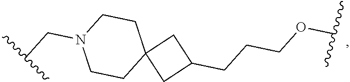 [SMILES: *CN1CCC2(CC1)CC(CCCO*)C2] | (577) |
| 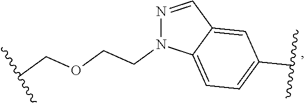 [SMILES: *COCCn1ncc2cc(*)ccc21] | (578) |
| 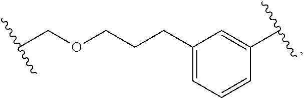 [SMILES: *COCCCc1cccc(*)c1] | (579) |
| 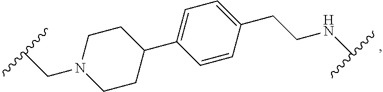 [SMILES: *CN1CCC(c2ccc(CCN*)cc2)CC1] | (580) |
| 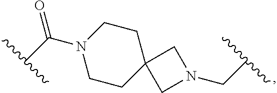 [SMILES: *CN1CC2(CCN(C(*)=O)CC2)C1] | (581) |
| 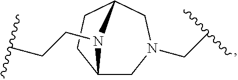 [SMILES: *CCN1[C@@H]2CC[C@H]1CN(C*)C2] | (582) |
| 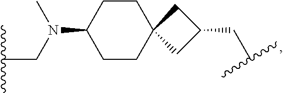 | (583) |
| 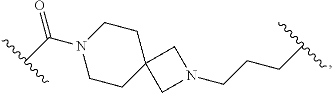 [SMILES: *CCCN1CC2(CCN(C(*)=O)CC2)C1] | (584) |
| 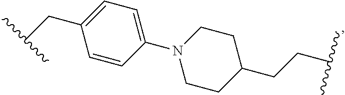 [SMILES: *CCC1CCN(c2ccc(C*)cc2)CC1] | (585) |
| 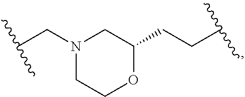 [SMILES: *CC[C@H]1CN(C*)CCO1] | (586) |
| 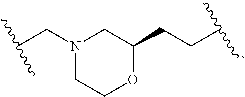 [SMILES: *CC[C@@H]1CN(C*)CCO1] | (587) |
| 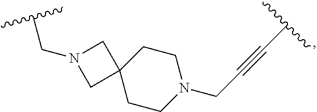 [SMILES: *C#CCN1CCC2(CC1)CN(C*)C2] | (588) |
| 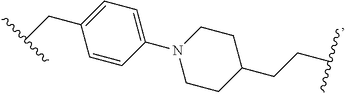 [SMILES: *CCC1CCN(c2ccc(C*)cc2)CC1] | (589) |
| 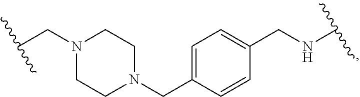 [SMILES: *CN1CCN(Cc2ccc(CN*)cc2)CC1] | (590) |
| 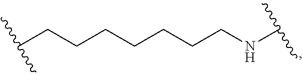 [SMILES: *CCCCCCCN*] | (591) |
| 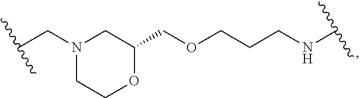 [SMILES: *CN1CCO[C@@H](COCCCN*)C1] | (592) |
| 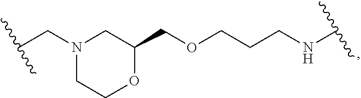 [SMILES: *CN1CCO[C@H](COCCCN*)C1] | (593) |
| 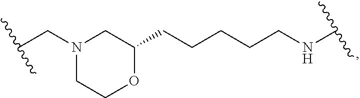 [SMILES: *CN1CCO[C@@H](CCCCCN*)C1] | (594) |
| 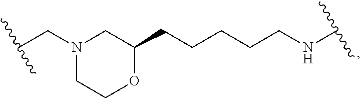 [SMILES: *CN1CCO[C@H](CCCCCN*)C1] | (595) |
| 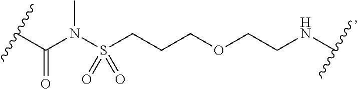 [SMILES: *NCCOCCCS(=O)(=O)N(C)C(*)=O] | (596) |
| 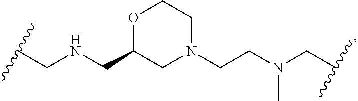 [SMILES: *CNC[C@@H]1CN(CCN(C)C*)CCO1] | (597) |
| 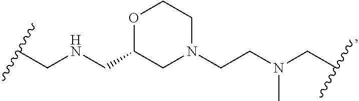 [SMILES: *CNC[C@H]1CN(CCN(C)C*)CCO1] | (598) |
| 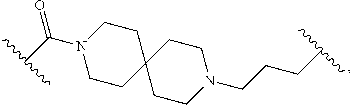 [SMILES: *CCCN1CCC2(CC1)CCN(C(*)=O)CC2] | (599) |
| 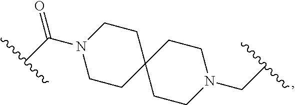 [SMILES: *CN1CCC2(CC1)CCN(C(*)=O)CC2] | (600) |
| 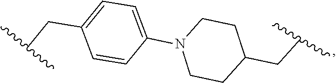 [SMILES: *Cc1ccc(N2CCC(C*)CC2)cc1] | (601) |
| 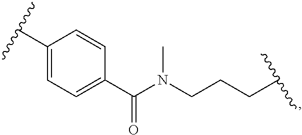 [SMILES: *CCCN(C)C(=O)c1ccc(*)cc1] | (602) |
| 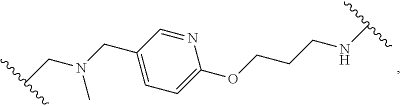 [SMILES: *CN(C)Cc1ccc(OCCCN*)nc1] | (603) |
| 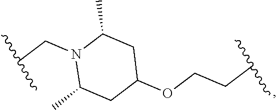 [SMILES: *CCOC1C[C@@H](C)N(C*)[C@@H](C)C1] | (604) |
| 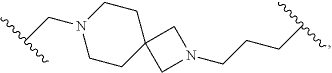 [SMILES: *CCCN1CC2(CCN(C*)CC2)C1] | (605) |
| 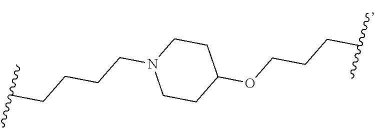 [SMILES: *CCCCN1CCC(OCCC*)CC1] | (606) |
| 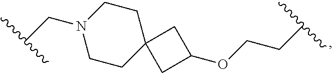 [SMILES: *CCOC1CC2(CCN(C*)CC2)C1] | (607) |
| 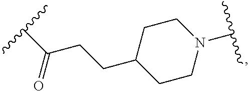 [SMILES: *C(=O)CCC1CCN(*)CC1] | (608) |
| 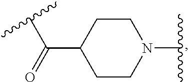 [SMILES: *C(=O)C1CCN(*)CC1] | (609) |
| 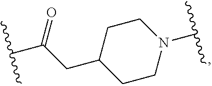 [SMILES: *C(=O)CC1CCN(*)CC1] | (610) |
| 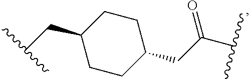 [SMILES: *C[C@H]1CC[C@H](CC(*)=O)CC1] | (611) |
| 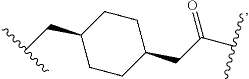 [SMILES: *C[C@H]1CC[C@@H](CC(*)=O)CC1] | (612) |
| 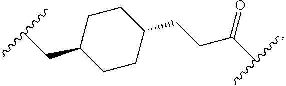 [SMILES: *C[C@H]1CC[C@H](CCC(*)=O)CC1] | (613) |
| 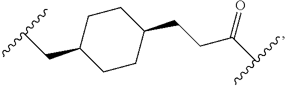 [SMILES: *C[C@H]1CC[C@@H](CCC(*)=O)CC1] | (614) |
| 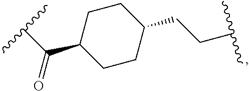 [SMILES: *CC[C@H]1CC[C@H](C(*)=O)CC1] | (615) |
| 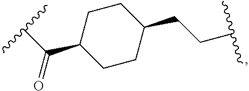 [SMILES: *CC[C@H]1CC[C@@H](C(*)=O)CC1] | (616) |
| 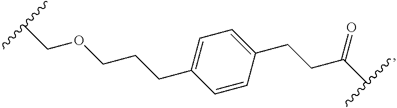 [SMILES: *COCCCc1ccc(CCC(*)=O)cc1] | (617) |
| 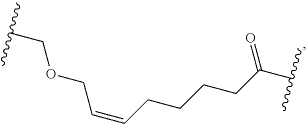 [SMILES: *COC/C=C\CCCCC(*)=O] | (618) |
| 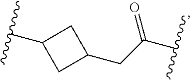 [SMILES: *C(=O)CC1CC(*)C1] | (619) |
| 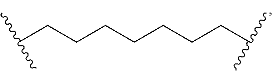 [SMILES: *CCCCCCC*] | (620) |
| 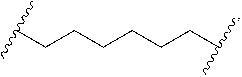 [SMILES: *CCCCCC*] | (621) |
| 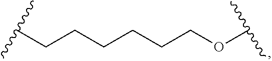 [SMILES: *CCCCCCO*] | (622) |
| 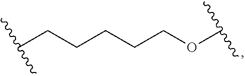 [SMILES: *CCCCCO*] | (623) |
| 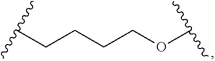 [SMILES: *CCCCO*] | (624) |
| 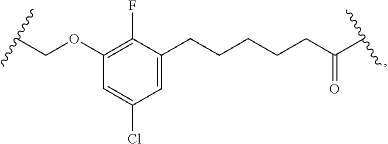 [SMILES: *COc1cc(Cl)cc(CCCCCC(*)=O)c1F] | (625) |
| 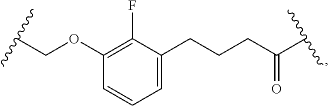 [SMILES: *COc1cccc(CCCC(*)=O)c1F] | (626) |
| 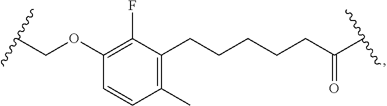 [SMILES: *COc1ccc(C)c(CCCCCC(*)=O)c1F] | (627) |
| 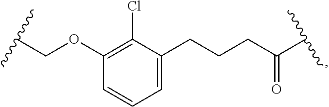 [SMILES: *COc1cccc(CCCC(*)=O)c1Cl] | (628) |
| 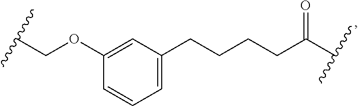 [SMILES: *COc1cccc(CCCCC(*)=O)c1] | (629) |
| 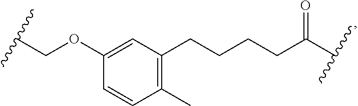 [SMILES: *COc1ccc(C)c(CCCCC(*)=O)c1] | (630) |
| 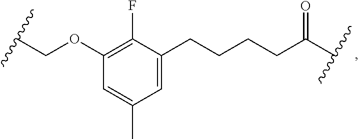 [SMILES: *COc1cc(C)cc(CCCCC(*)=O)c1F] | (631) |
| 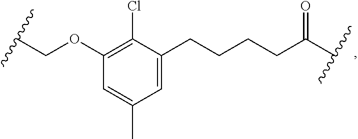 [SMILES: *COc1cc(C)cc(CCCCC(*)=O)c1Cl] | (632) |
| 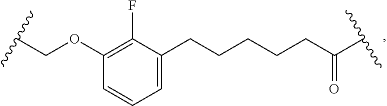 [SMILES: *COc1cccc(CCCCCC(*)=O)c1F] | (633) |
| 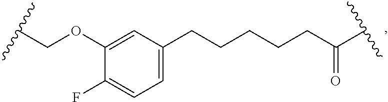 [SMILES: *COc1cc(CCCCCC(*)=O)ccc1F] | (634) |
| 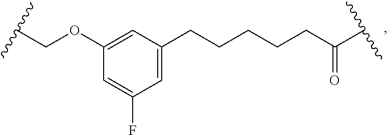 [SMILES: *COc1cc(F)cc(CCCCCC(*)=O)c1] | (635) |
| 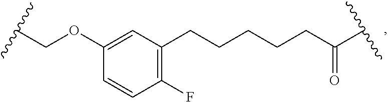 [SMILES: *COc1ccc(F)c(CCCCCC(*)=O)c1] | (636) |
| 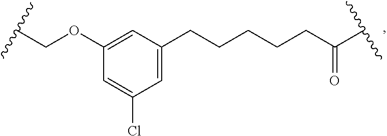 [SMILES: *COc1cc(Cl)cc(CCCCCC(*)=O)c1] | (637) |
| 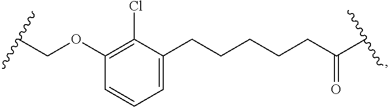 [SMILES: *COc1cccc(CCCCCC(*)=O)c1Cl] | (638) |
| 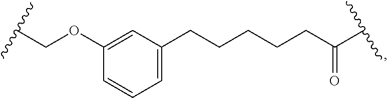 [SMILES: *COc1cccc(CCCCCC(*)=O)c1] | (639) |
| 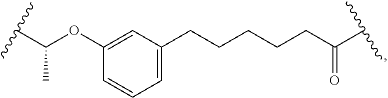 [SMILES: *C(=O)CCCCCc1cccc(O[C@@H](*)C)c1] | (640) |
| 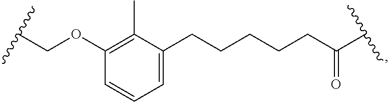 [SMILES: *COc1cccc(CCCCCC(*)=O)c1C] | (641) |
| 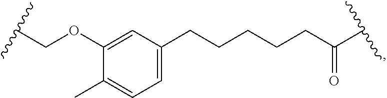 [SMILES: *COc1cc(CCCCCC(*)=O)ccc1C] | (642) |
| 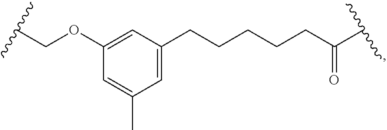 [SMILES: *COc1cc(C)cc(CCCCCC(*)=O)c1] | (643) |
| 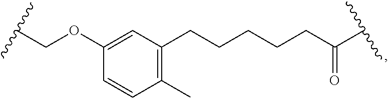 [SMILES: *COc1ccc(C)c(CCCCCC(*)=O)c1] | (644) |
| 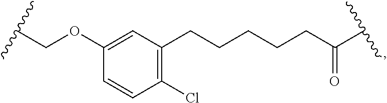 [SMILES: *COc1ccc(Cl)c(CCCCCC(*)=O)c1] | (645) |
| 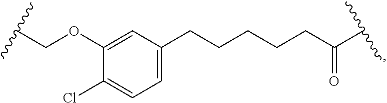 [SMILES: *COc1cc(CCCCCC(*)=O)ccc1Cl] | (646) |
| 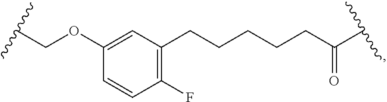 [SMILES: *COc1ccc(F)c(CCCCCC(*)=O)c1] | (647) |
| 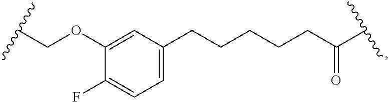 [SMILES: *COc1cc(CCCCCC(*)=O)ccc1F] | (648) |
| 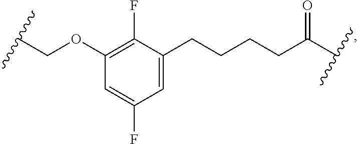 [SMILES: *COc1cc(F)cc(CCCCC(*)=O)c1F] | (649) |
| 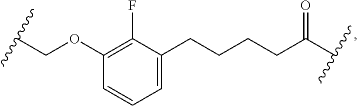 [SMILES: *COc1cccc(CCCCC(*)=O)c1F] | (650) |
| 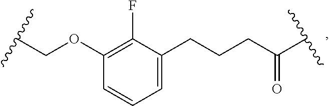 [SMILES: *COc1cccc(CCCC(*)=O)c1F] | (651) |
| 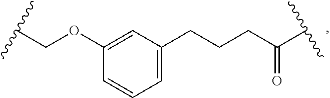 [SMILES: *COc1cccc(CCCC(*)=O)c1] | (652) |
| 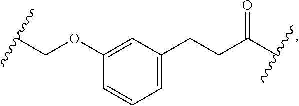 [SMILES: *COc1cccc(CCC(*)=O)c1] | (653) |
| 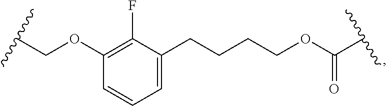 [SMILES: *COc1cccc(CCCCOC(*)=O)c1F] | (654) |
| 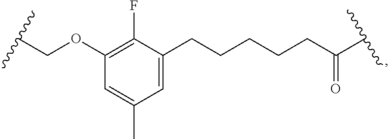 [SMILES: *COc1cc(C)cc(CCCCCC(*)=O)c1F] | (655) |
| 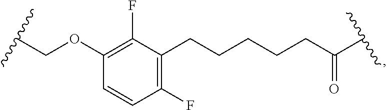 [SMILES: *COc1ccc(F)c(CCCCCC(*)=O)c1F] | (656) |
| 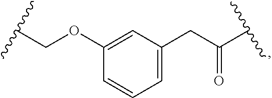 [SMILES: *COc1cccc(CC(*)=O)c1] | (657) |
| 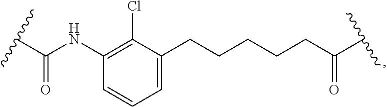 [SMILES: *C(=O)CCCCCc1cccc(NC(*)=O)c1Cl] | (658) |
| 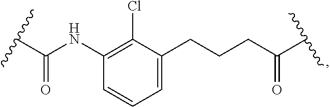 [SMILES: *C(=O)CCCc1cccc(NC(*)=O)c1Cl] | (659) |
| 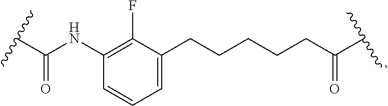 [SMILES: *C(=O)CCCCCc1cccc(NC(*)=O)c1F] | (660) |
| 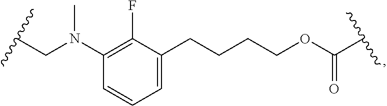 [SMILES: *CN(C)c1cccc(CCCCOC(*)=O)c1F] | (661) |
| 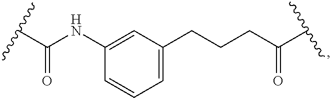 [SMILES: *C(=O)CCCc1cccc(NC(*)=O)c1] | (662) |
| 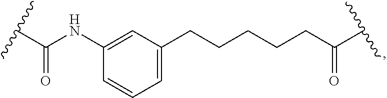 [SMILES: *C(=O)CCCCCc1cccc(NC(*)=O)c1] | (663) |
| 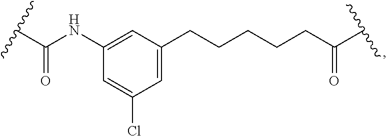 [SMILES: *C(=O)CCCCCc1cc(Cl)cc(NC(*)=O)c1] | (664) |
| 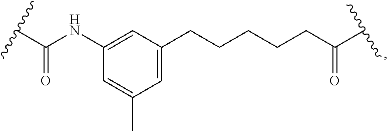 [SMILES: *C(=O)CCCCCc1cc(C)cc(NC(*)=O)c1] | (665) |
| 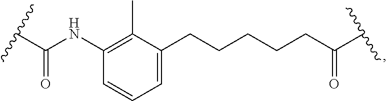 [SMILES: *C(=O)CCCCCc1cccc(NC(*)=O)c1C] | (666) |
| 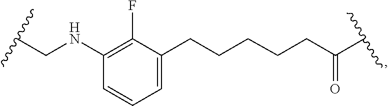 [SMILES: *CNc1cccc(CCCCCC(*)=O)c1F] | (667) |
| 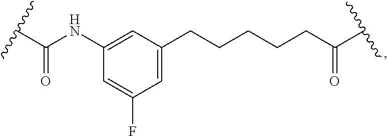 [SMILES: *C(=O)CCCCCc1cc(F)cc(NC(*)=O)c1] | (668) |
| 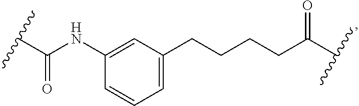 [SMILES: *C(=O)CCCCc1cccc(NC(*)=O)c1] | (669) |
| 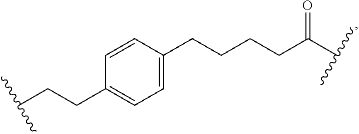 [SMILES: *CCc1ccc(CCCCC(*)=O)cc1] | (670) |
| 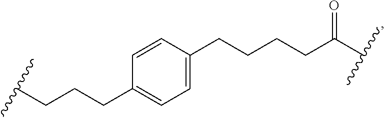 [SMILES: *CCCc1ccc(CCCCC(*)=O)cc1] | (671) |
| 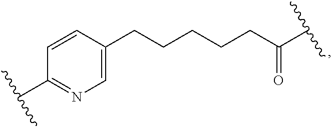 [SMILES: *C(=O)CCCCCc1ccc(*)nc1] | (672) |
| 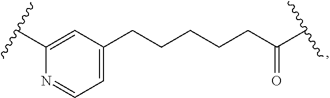 [SMILES: *C(=O)CCCCCc1ccnc(*)c1] | (673) |
| 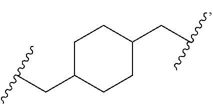 [SMILES: *CC1CCC(C*)CC1] | (674) |
| 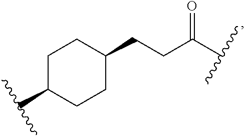 [SMILES: *C(=O)CC[C@H]1CC[C@@H](*)CC1] | (675) |
| 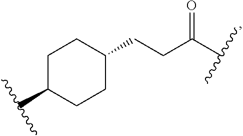 [SMILES: *C(=O)CC[C@H]1CC[C@H](*)CC1] | (676) |
| 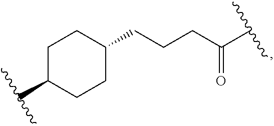 [SMILES: *C(=O)CCC[C@H]1CC[C@H](*)CC1] | (677) |
| 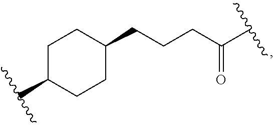 [SMILES: *C(=O)CCC[C@H]1CC[C@@H](*)CC1] | (678) |
| 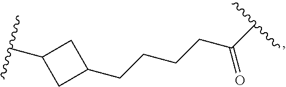 [SMILES: *C(=O)CCCCC1CC(*)C1] | (679) |
| 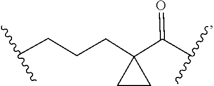 [SMILES: *CCCC1(C(*)=O)CC1] | (680) |

or any one of the linkers described in the
Linker section above.

In certain embodiments, the present invention provides methods of degrading STAT6, or a mutant thereof, or methods of treating a STAT6-mediated disease, disorder, or condition comprising administering a compound of Formula I-a″″-1, as a compound of Formula I-bb:

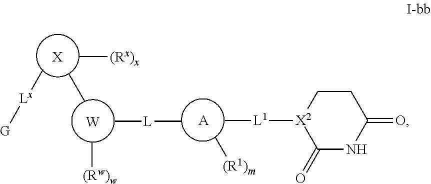

or a pharmaceutically acceptable salt thereof, wherein L, L^{x}, L¹, Ring A, Ring W, Ring X, G, R^{w}, R^{x}, R¹, X², m, w, and x are as defined above and described herein both individually and in combination.

In some embodiments, methods of degrading STAT6, or a mutant thereof, or methods of treating a STAT6-mediated disease, disorder, or condition comprising administering a compound is a compound of Formula I-bb:

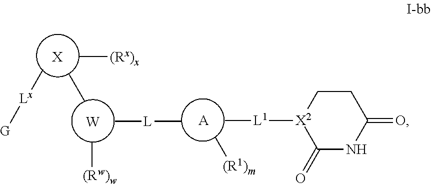

or a pharmaceutically acceptable salt thereof, wherein each of R, w, R^{X}, x, R^{y}, y, and L is as defined and described above and herein, and wherein:
X² is N or CH;
L¹ is a covalent bond, —C(O)—, —NR—, —O—, —S—, —S(O)₂, —NRC(O)—, or —C(O)NR—:
Ring A is phenylenyl or a 5 to 10-membered saturated or partially unsaturated monocyclic or bicyclic heterocyclylenyl or heteroarylenyl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂—S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
G is hydrogen, halogen, or

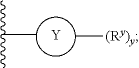

and
Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

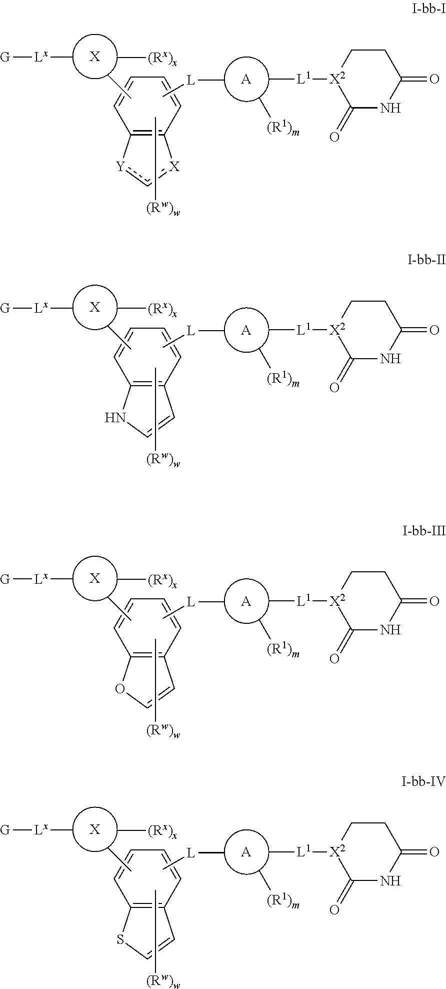

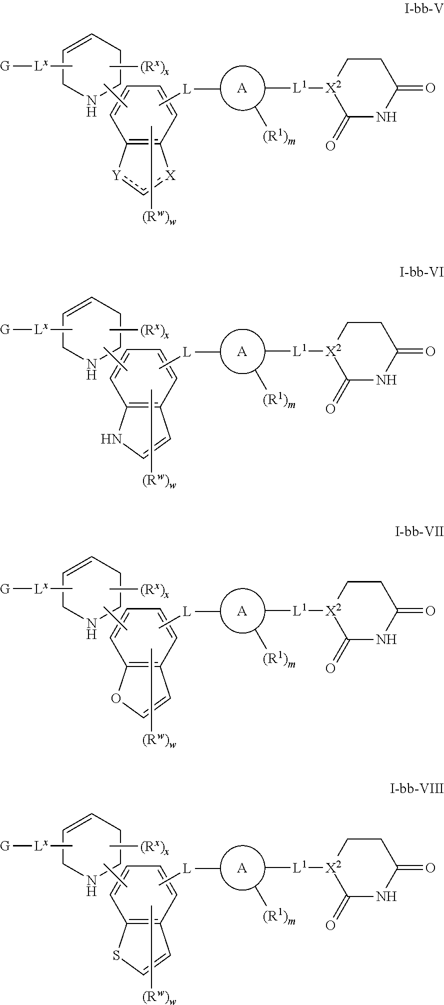

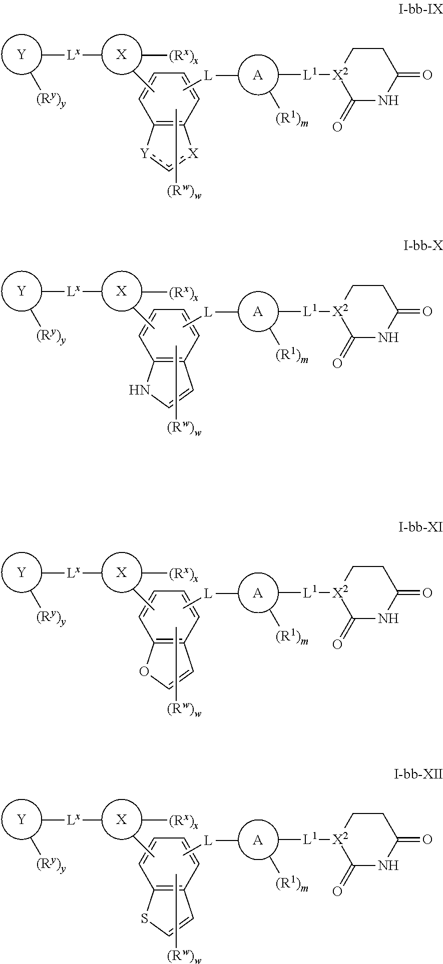

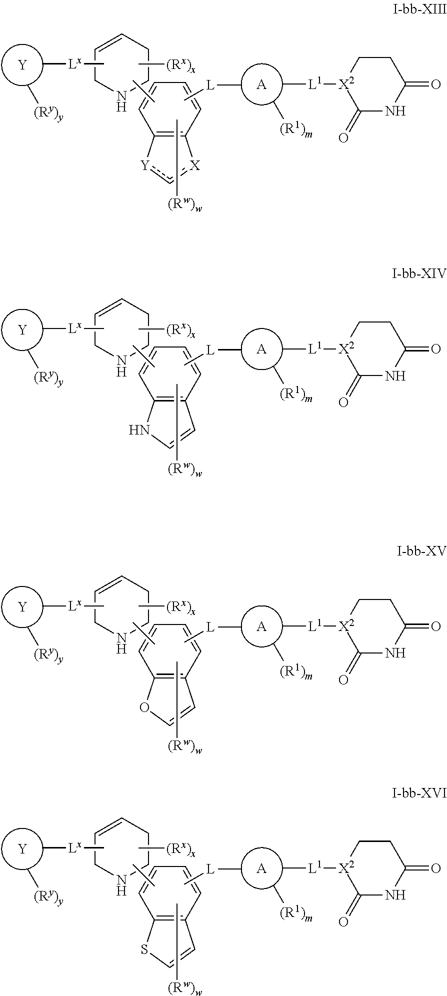

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

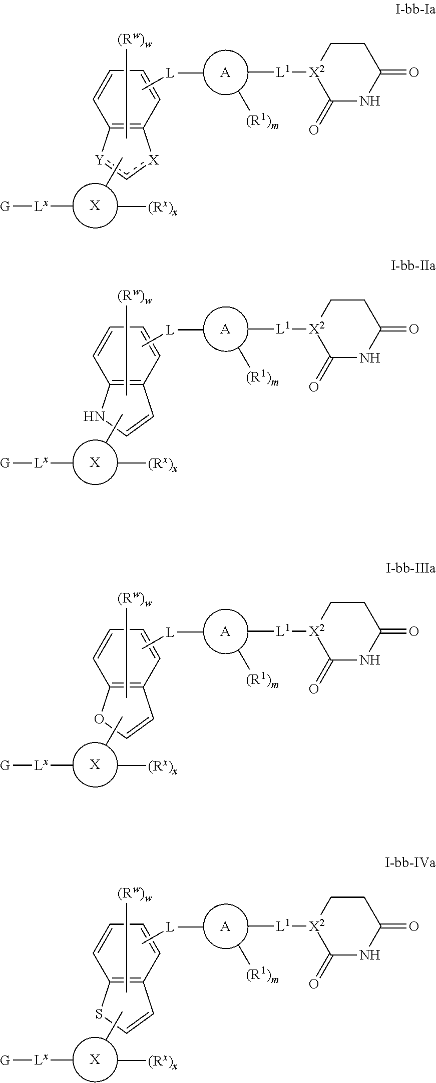

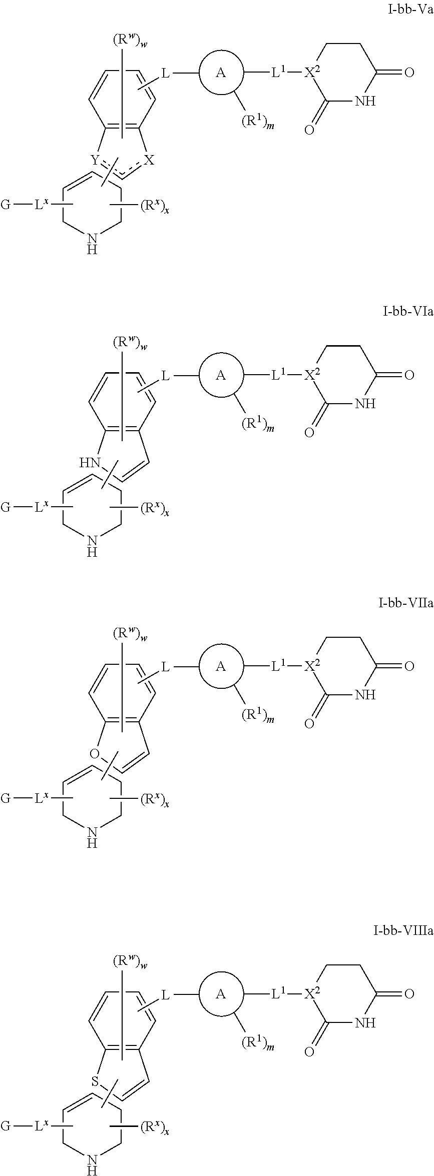

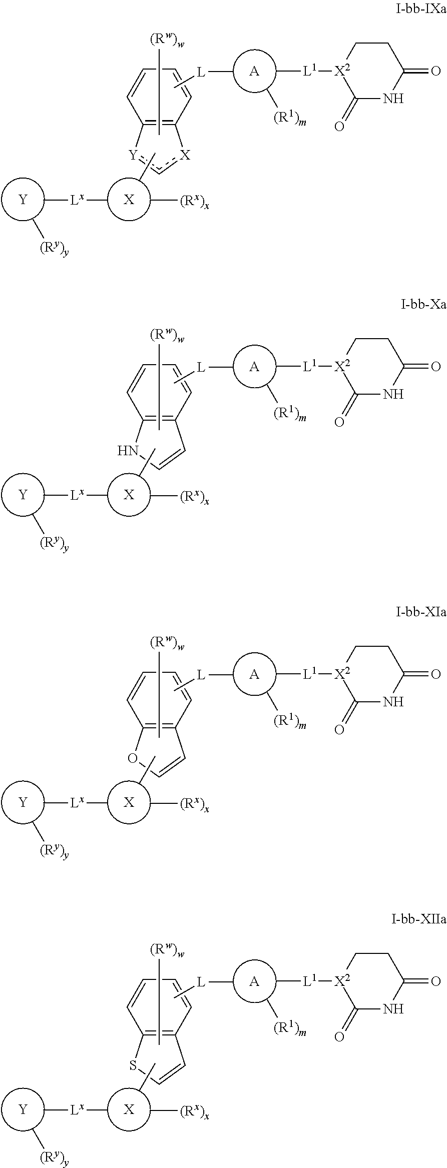

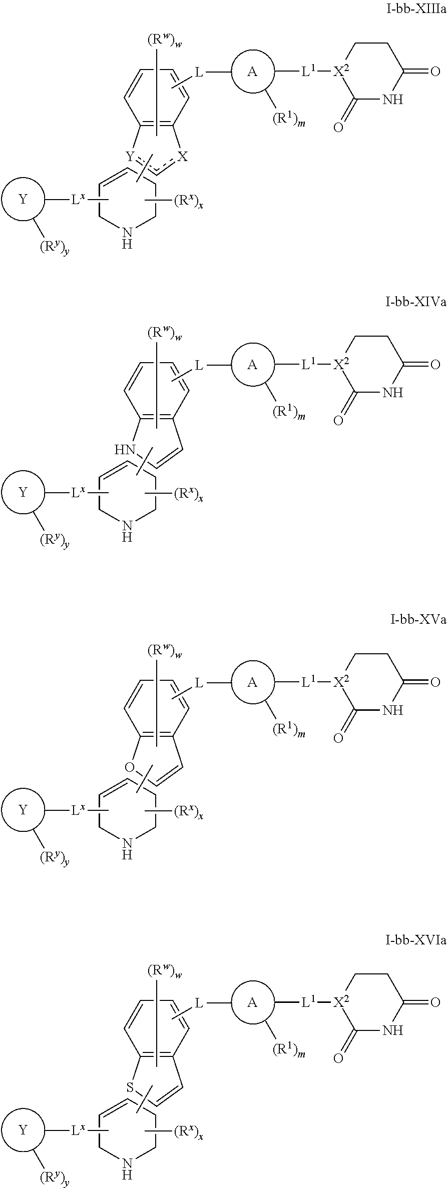

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

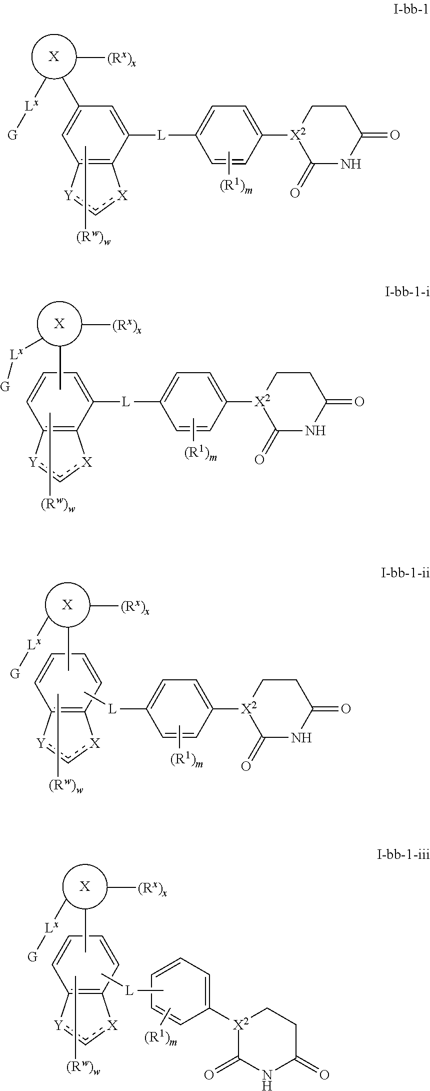

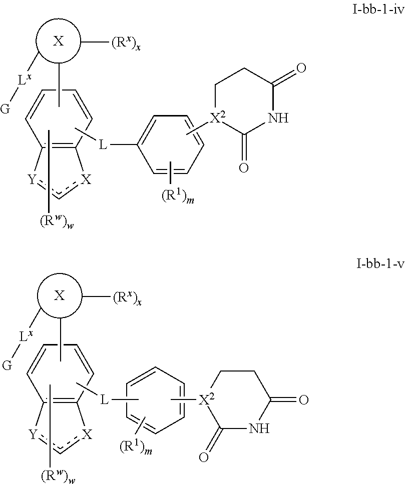

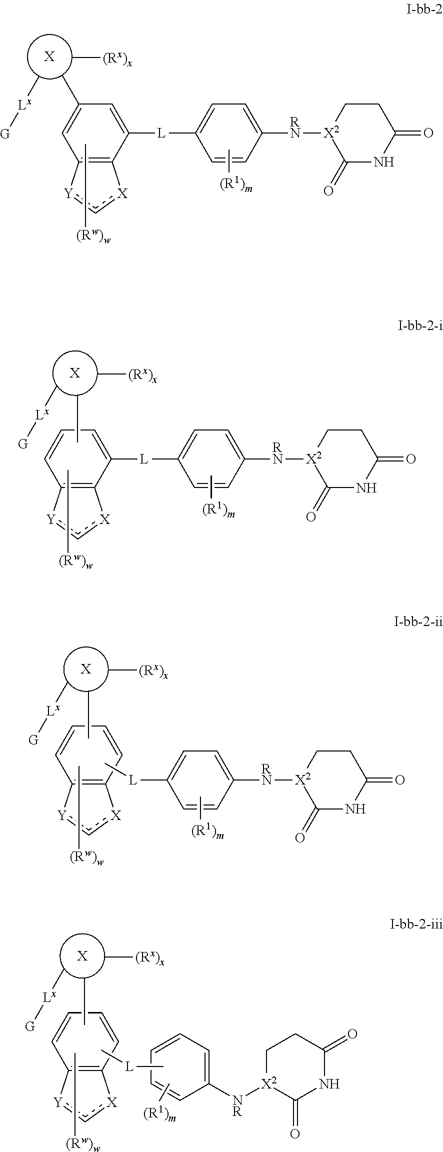

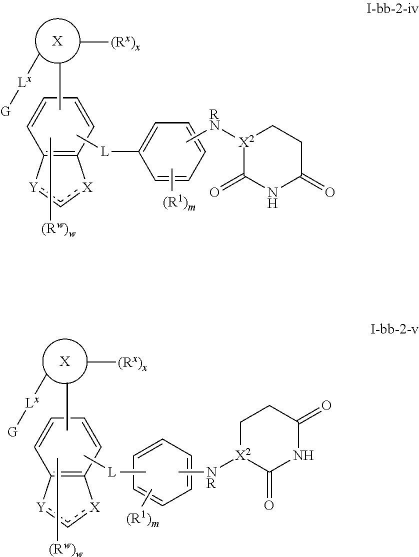

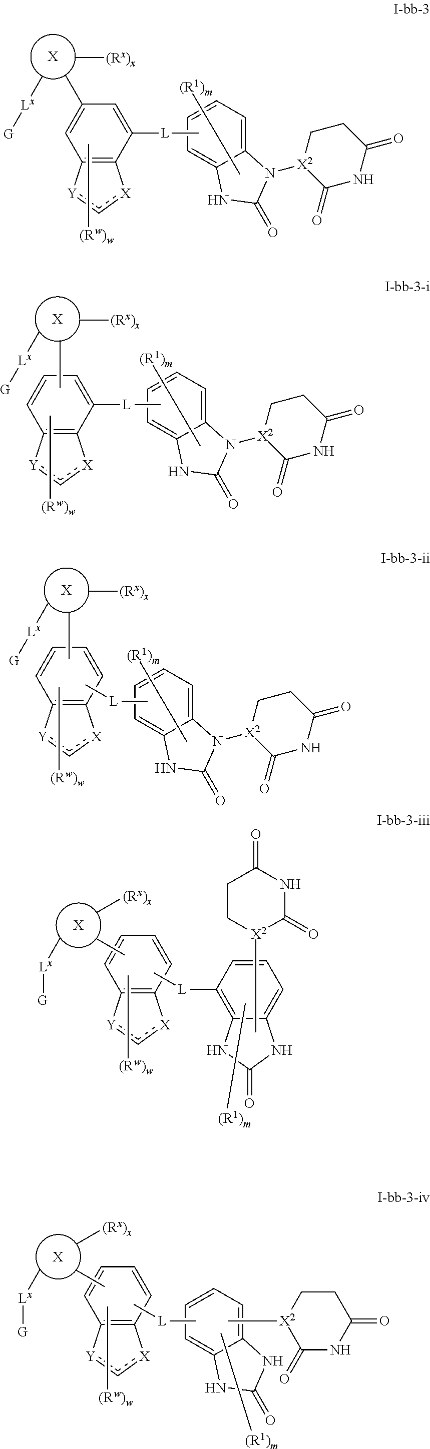

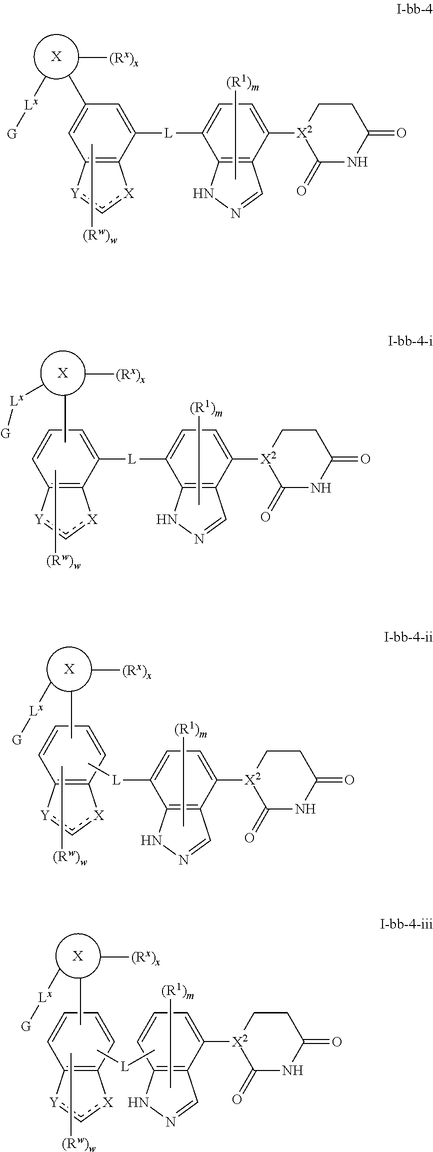

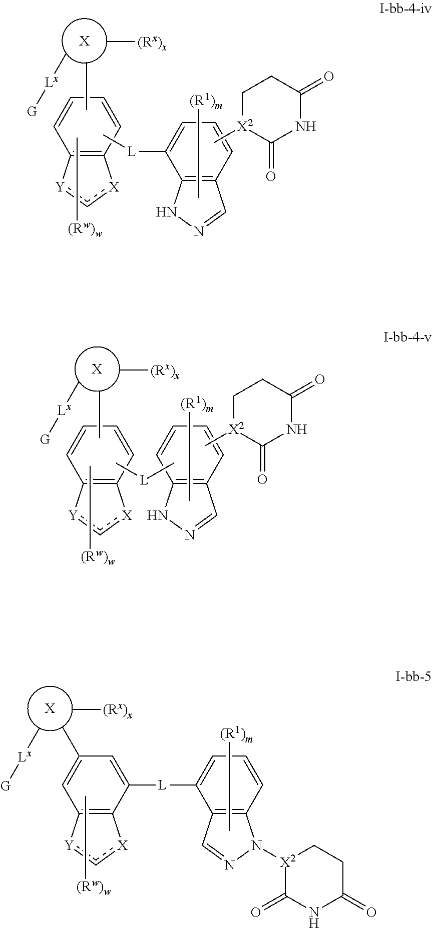

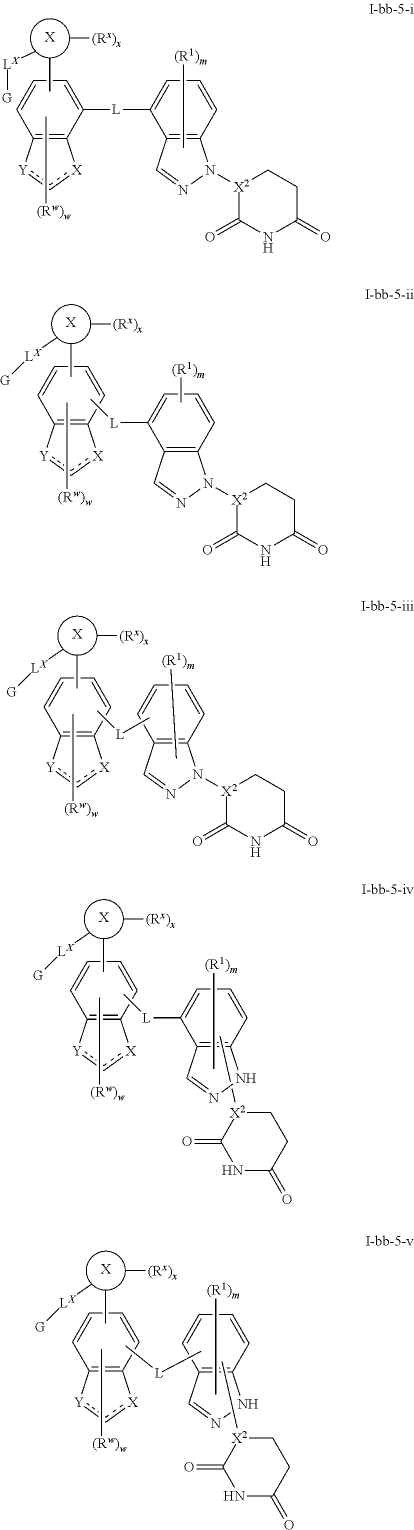

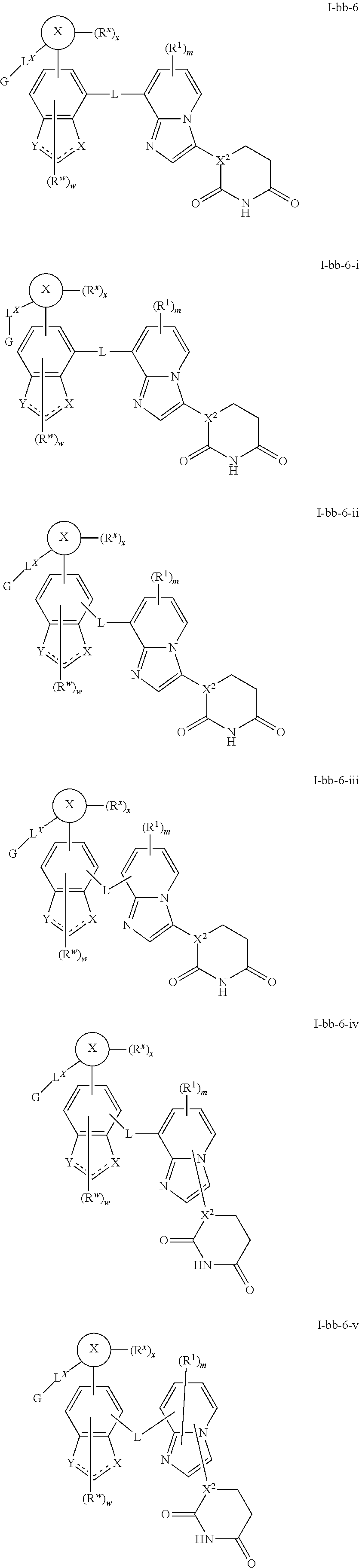

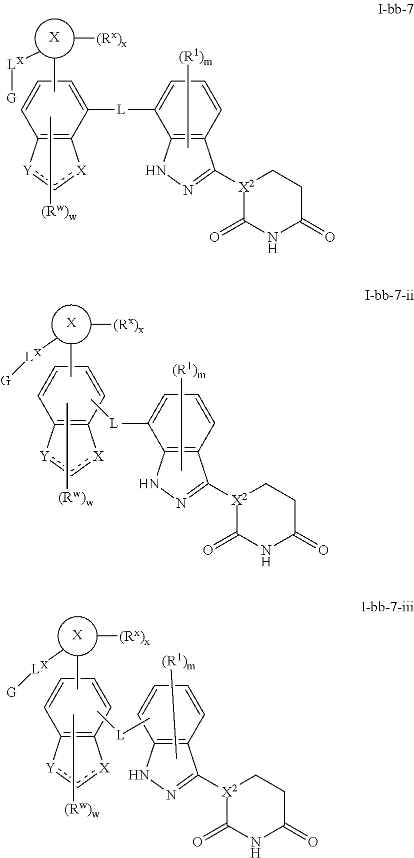

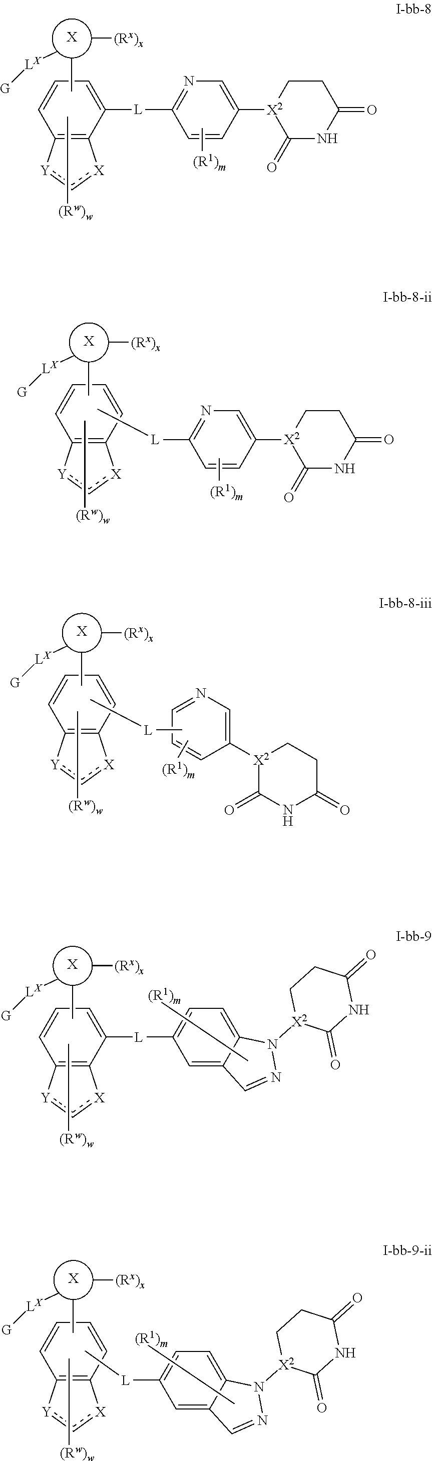

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

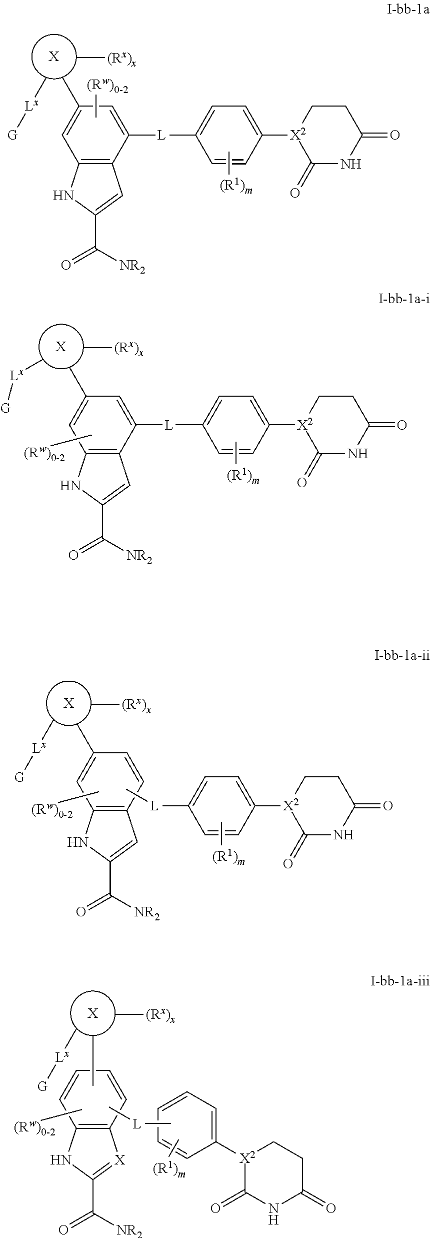

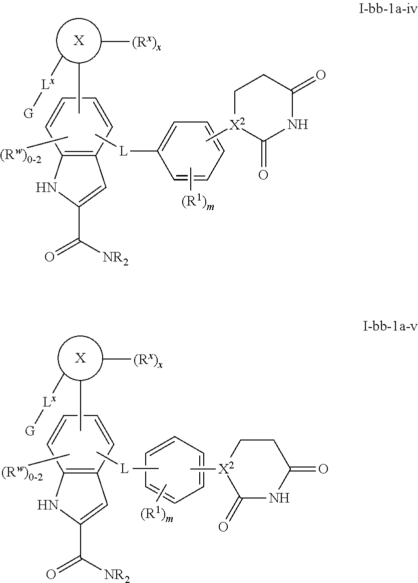

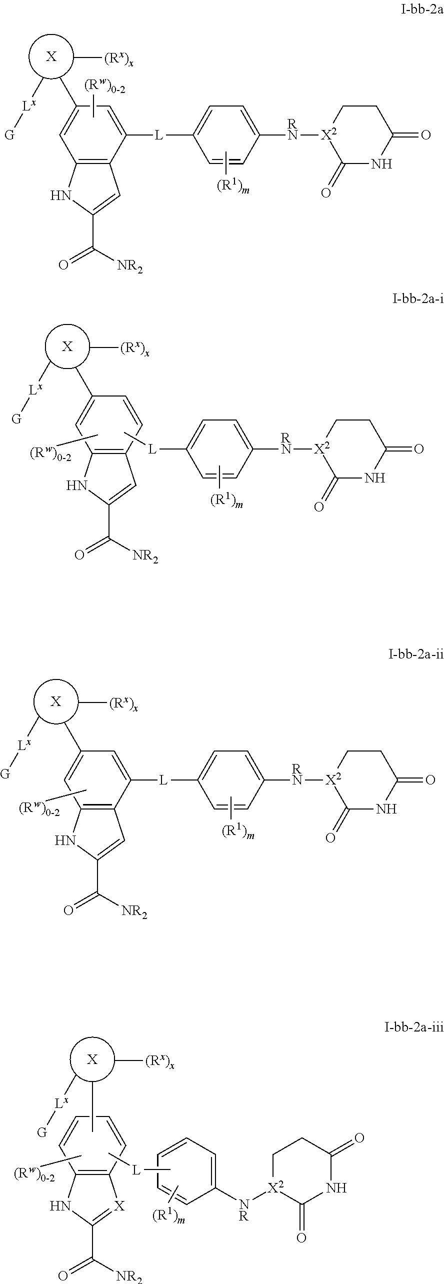

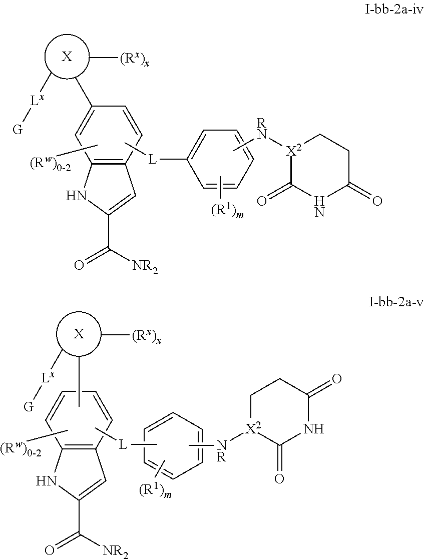

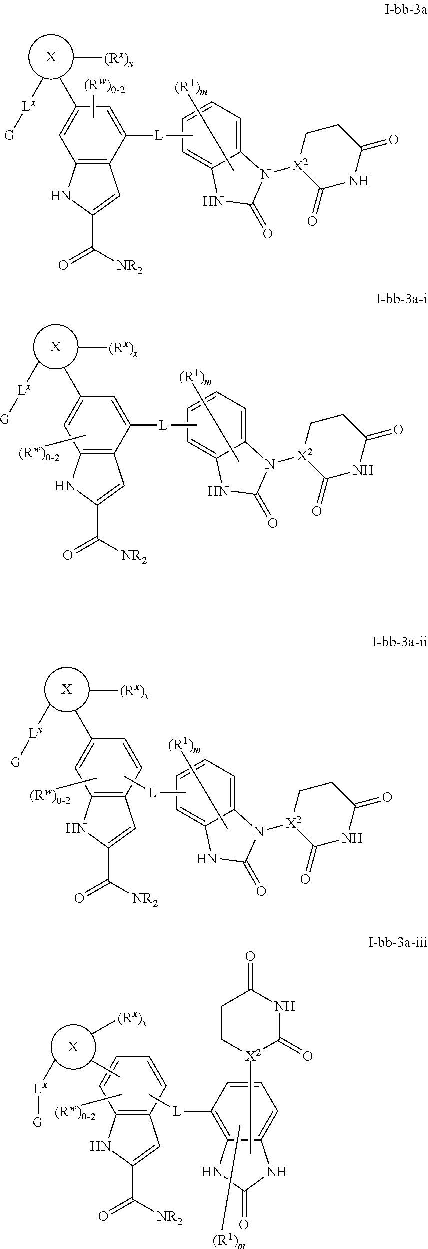

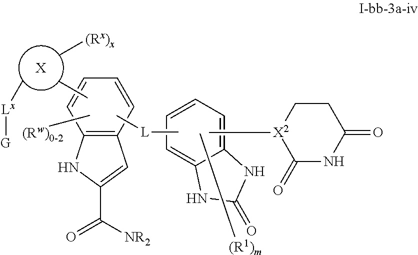

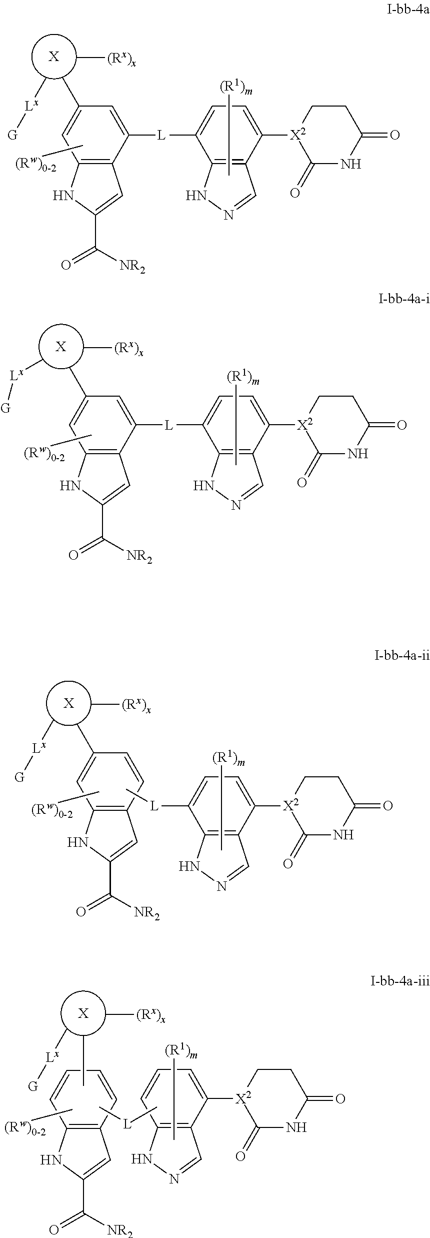

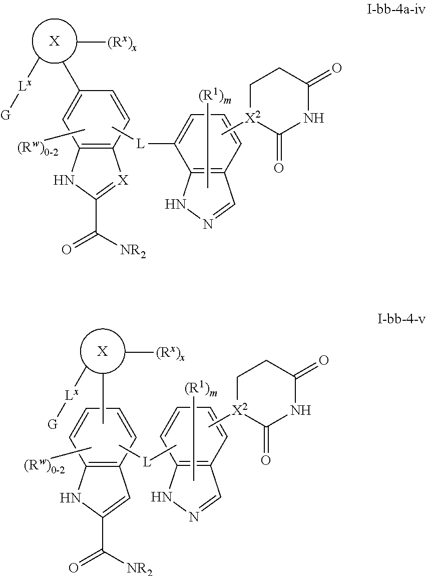

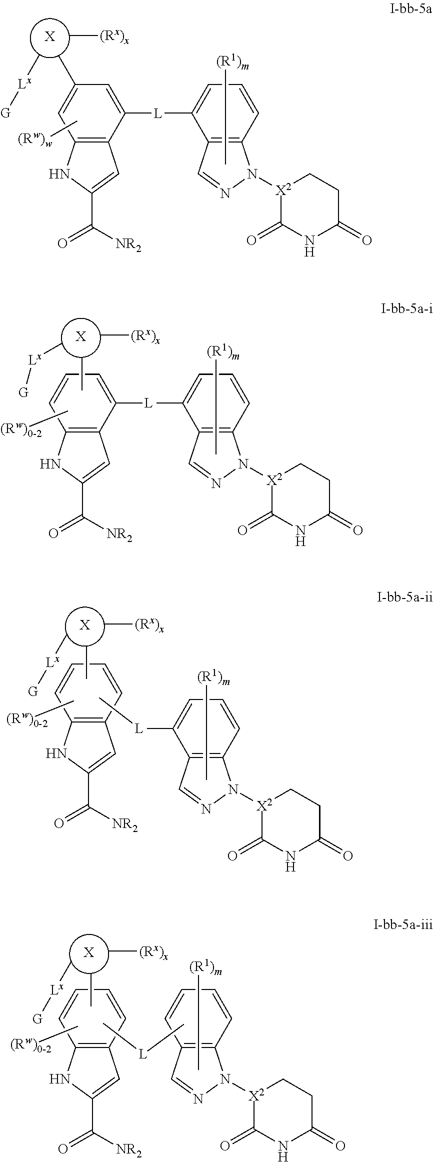

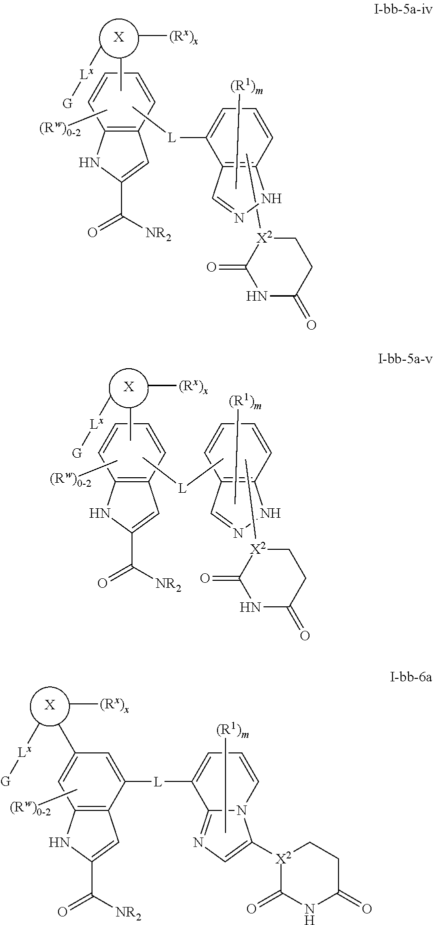

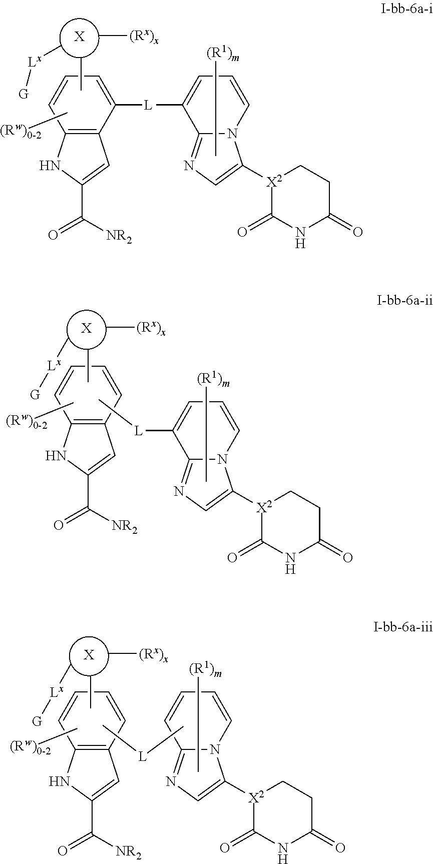

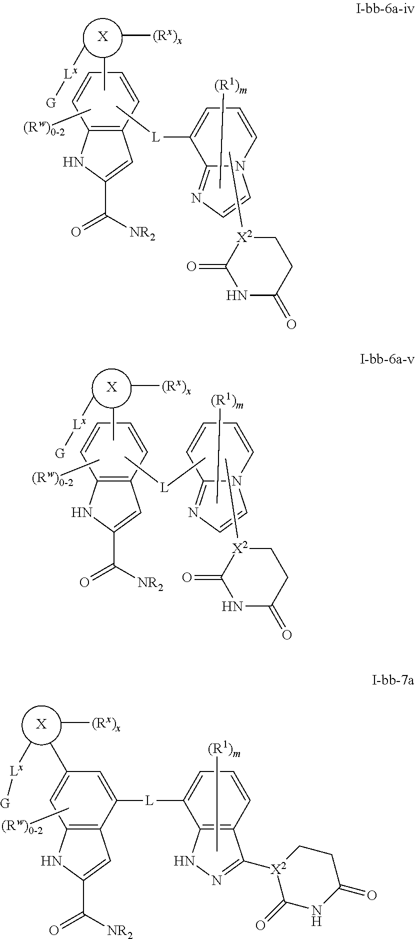

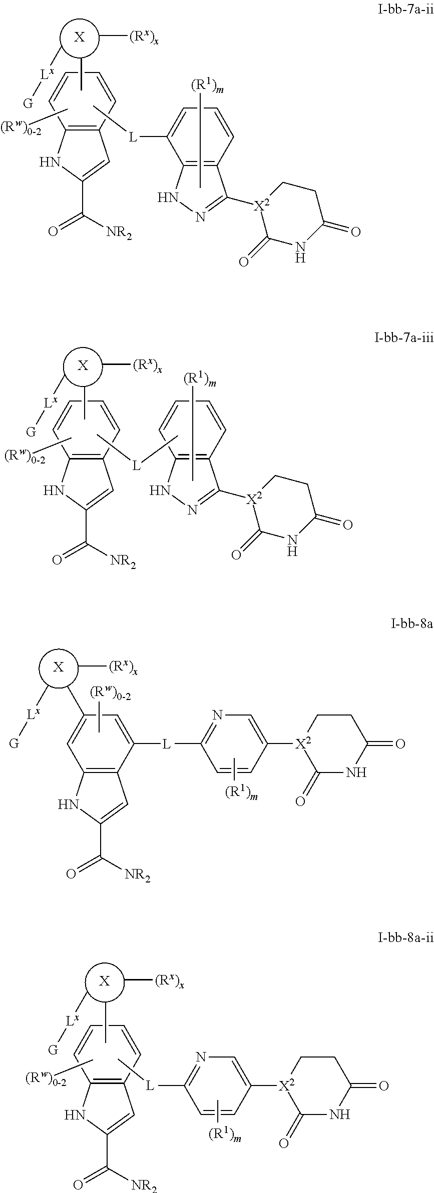

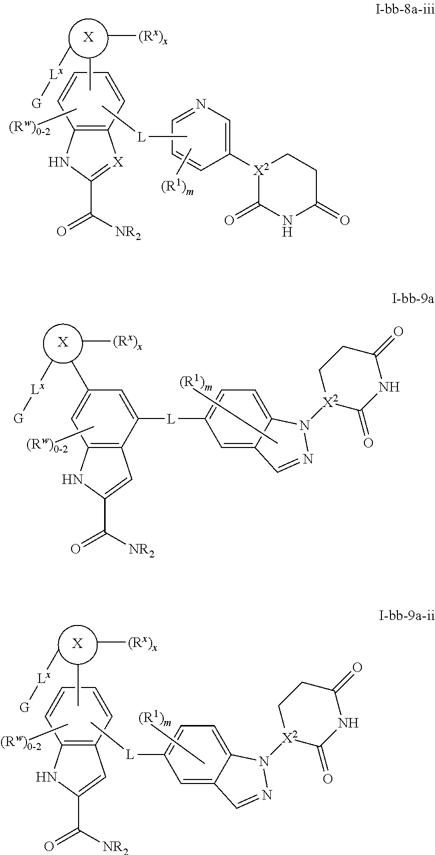

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

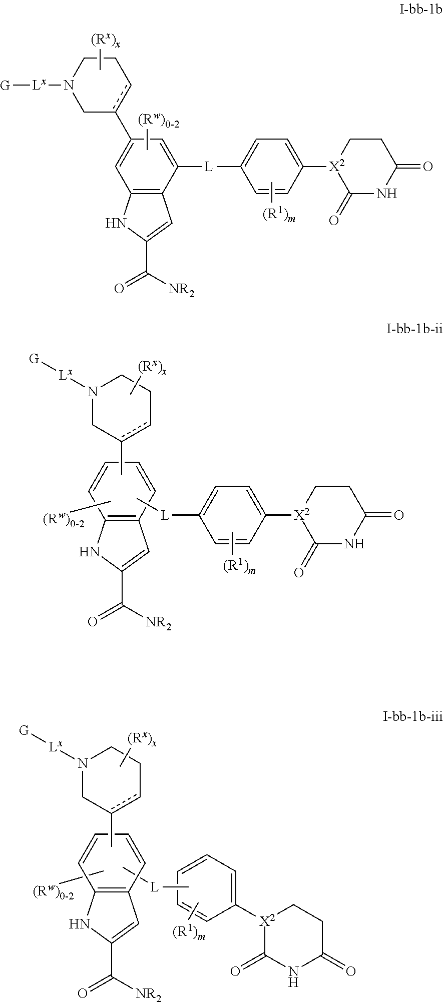

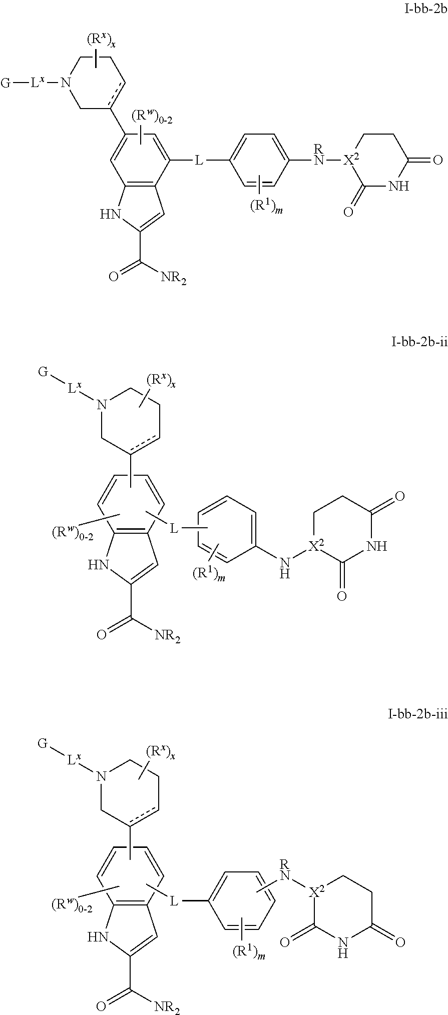

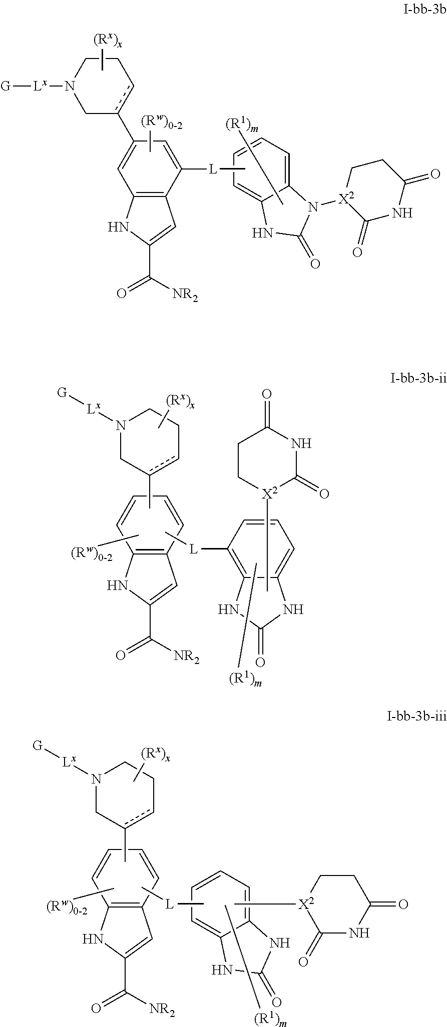

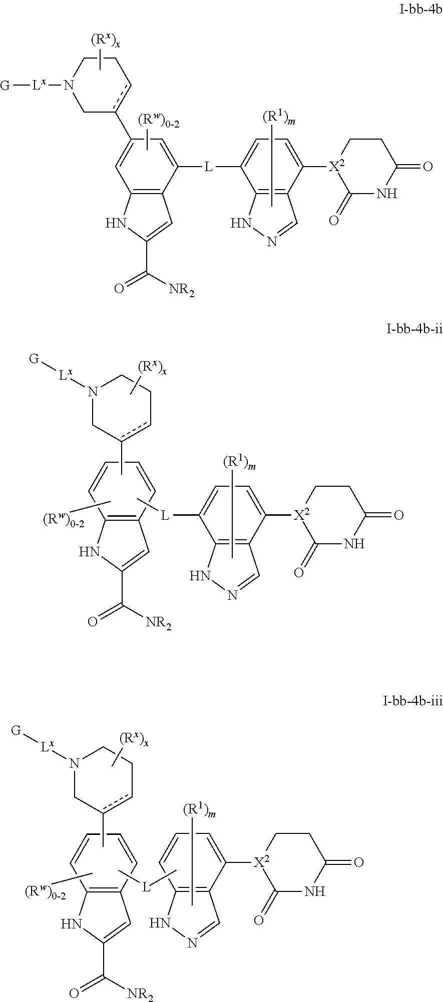

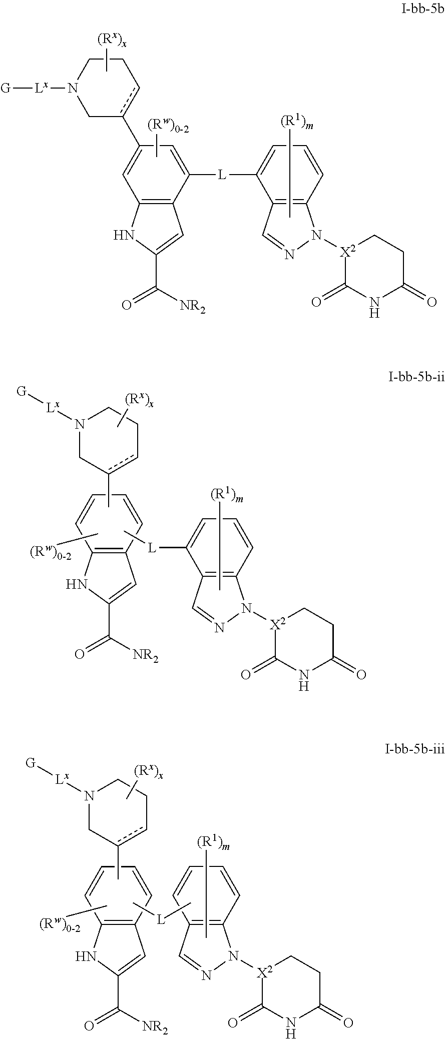

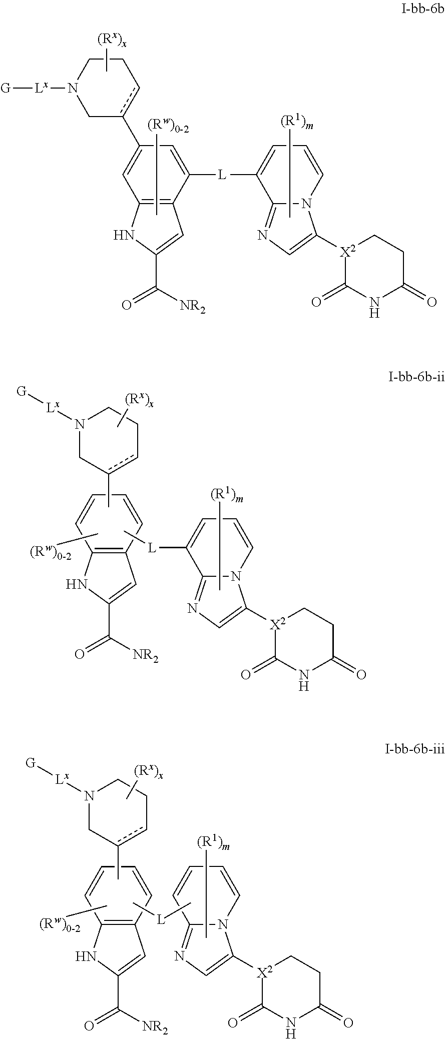

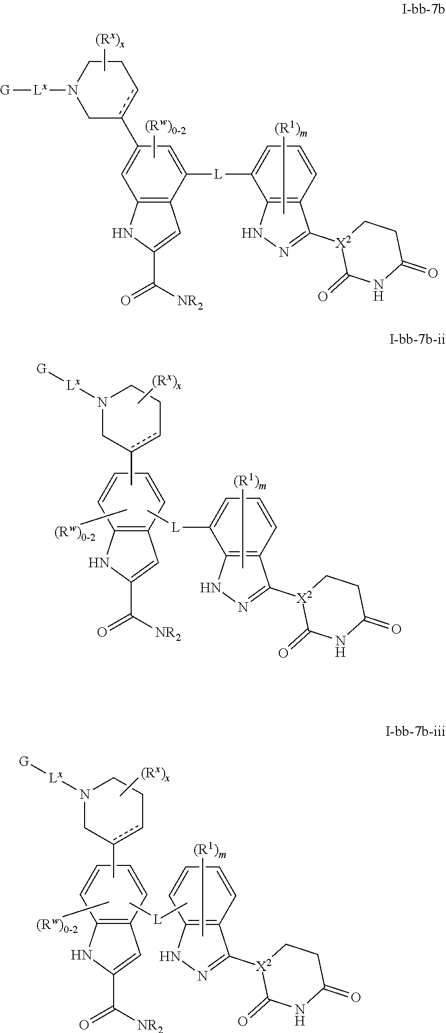

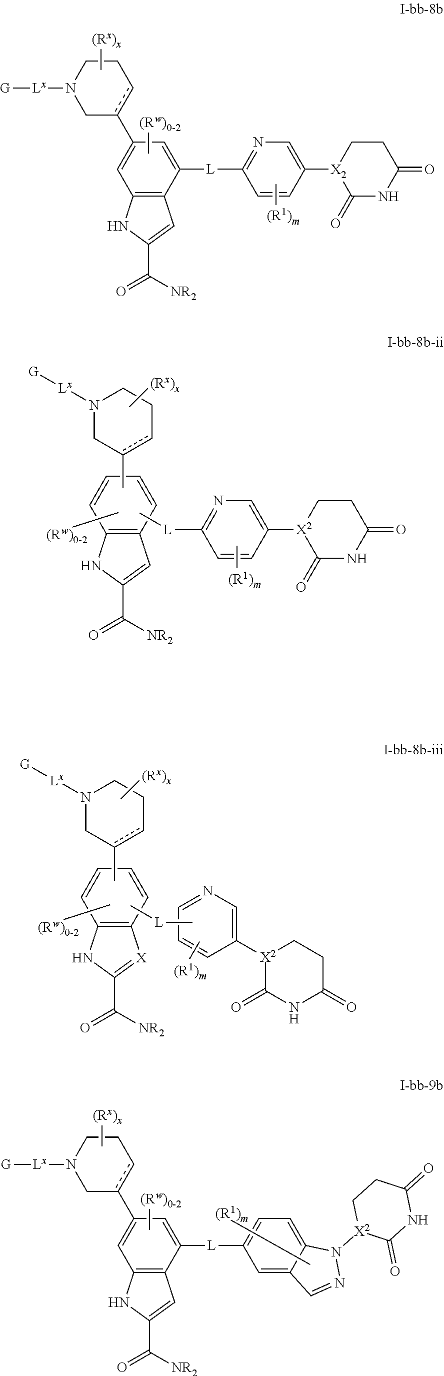

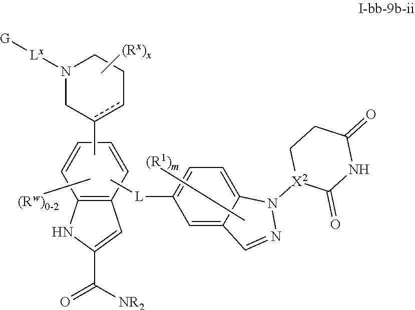

or a pharmaceutically acceptable salt thereof, wherein each variable is as defined above and described herein both individually and in combination, and wherein [symbol] represents a single or double bond.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

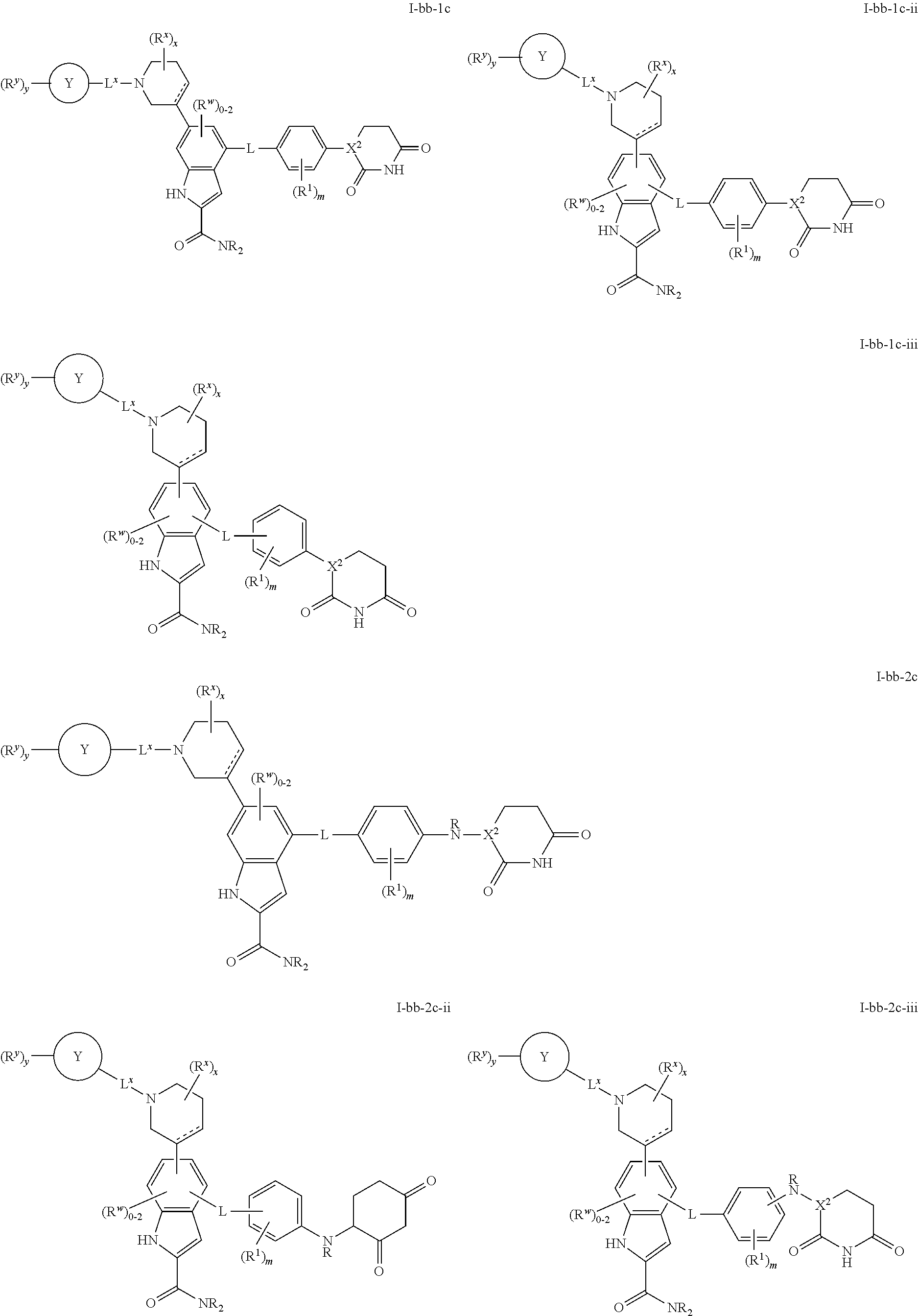

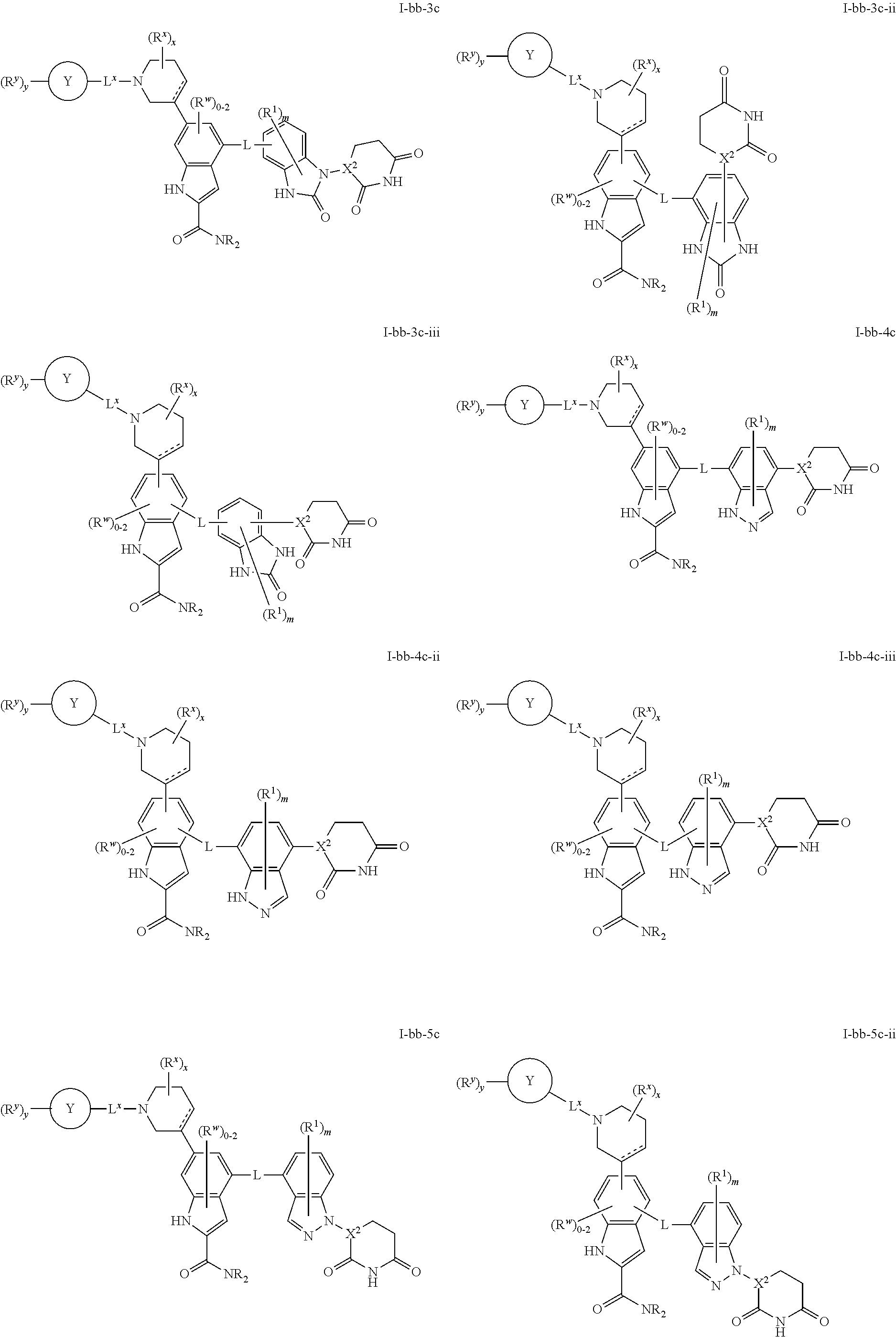

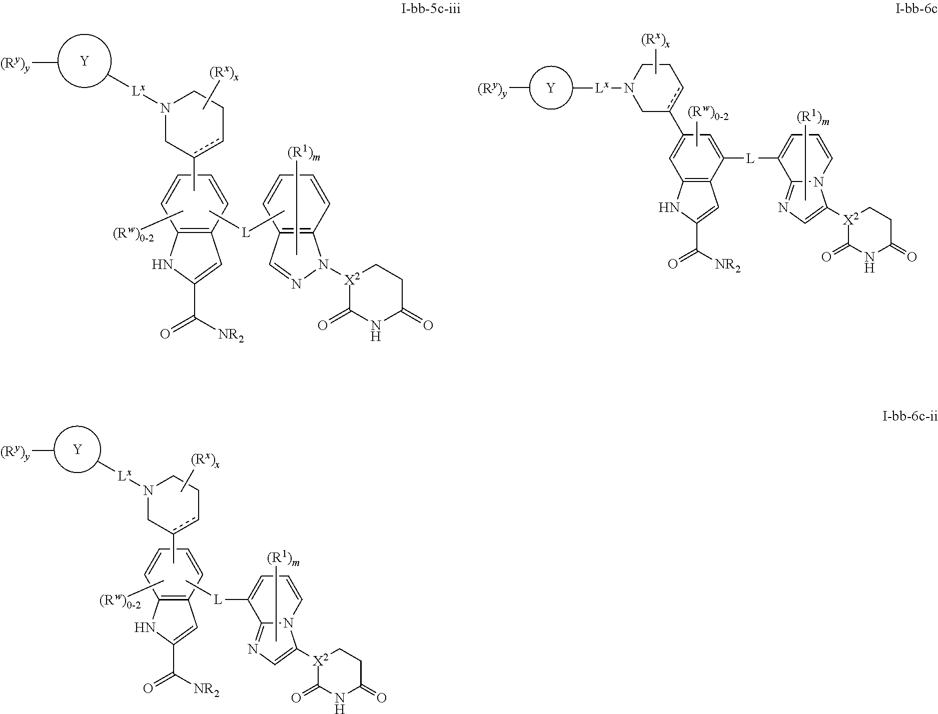

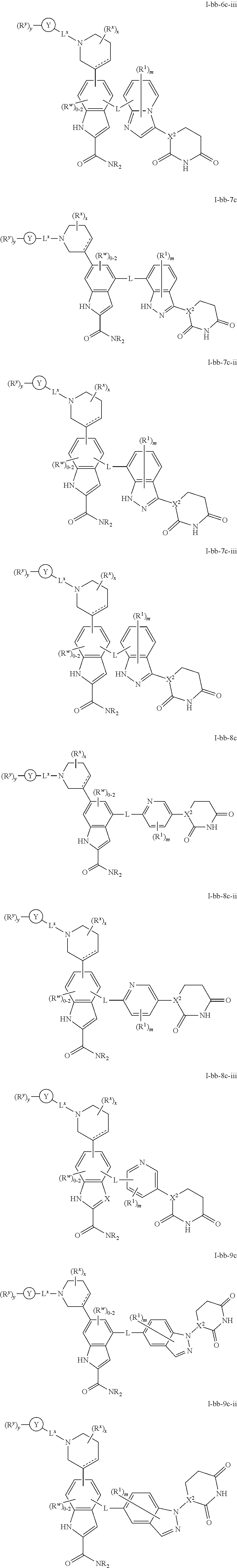

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of formula I-bb as a compound of any one of the following formulae:

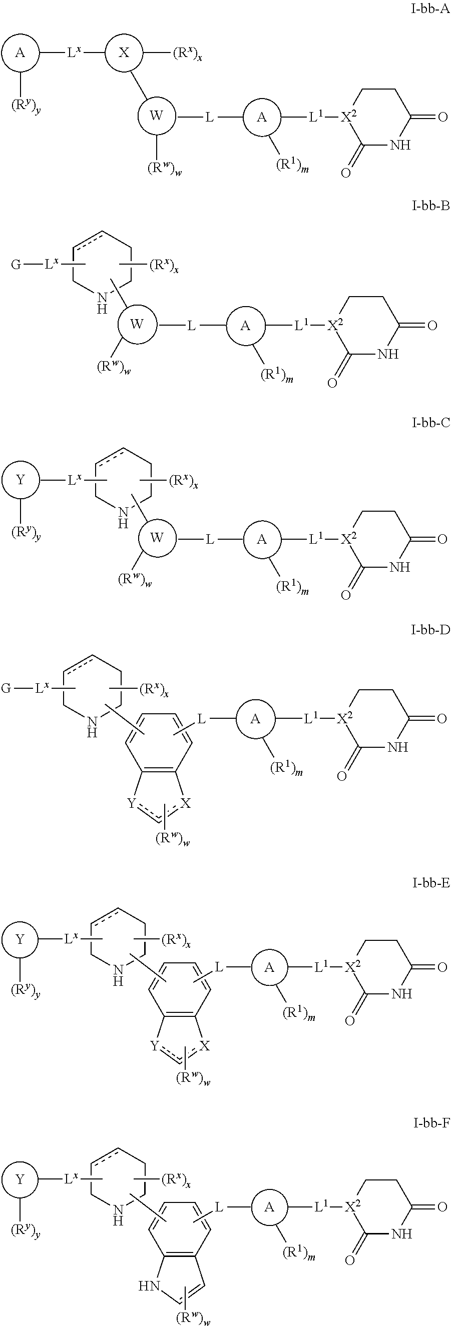

or a pharmaceutically acceptable salt thereof, wherein each variable is as defined above and described herein both individually and in combination:

In some embodiments, the present invention provides methods of degrading STAT6, or a mutant thereof, or methods of treating a STAT6-mediated disease, disorder, or condition comprising administering a compound of any one of the following formulae:

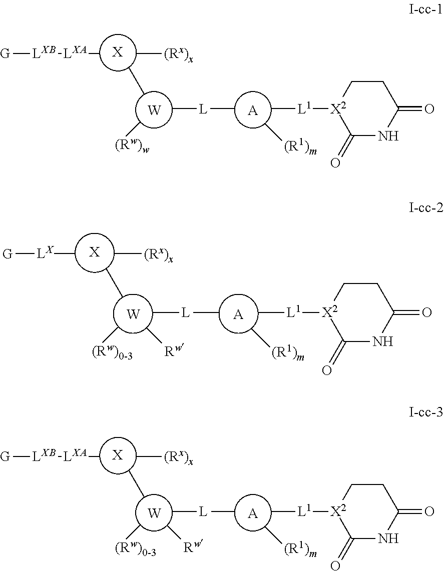

or a pharmaceutically acceptable salt thereof, wherein each Ring W, Ring X, R^{w}, R^{x}, w, x, G, L, L^{x}, Ring A, R¹, m, L¹, L^{XA}, L^{XB}, and X² is as defined above and described herein both individually and in combination, and wherein:
R^{w′} is R^{w}, wherein R^{w} as defined above and described herein.

In some embodiments, R^{w′} is —S(O)R, —S(O)₂R, —S(O)(NR)R, —S(O)₂NR₂, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, —NRS(O)₂R, or an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur.

In some embodiments, R^{w} is —S(O)₂R. In some embodiments, R^{w} is —S(O)₂NR₂. In some embodiments, R^{w} is —S(O)(NR)R. In some embodiments, R^{w} is —S(O)R. In some embodiments, R^{w} is —C(O)R. In some embodiments, R^{w} is —C(O)OR. In some embodiments, R^{w} is —C(O)NR₂. In some embodiments, R^{w} is —C(O)NROR. In some embodiments, R^{w} is —OC(O)R. In some embodiments, R^{w} is —OC(O)NR₂. In some embodiments, R^{w} is —P(O)R₂. In some embodiments, R^{w} is —P(O)(OR)₂. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —NRC(O)OR. In some embodiments, R^{W} is —NRC(O)R. In some embodiments, R^{w} is —NRC(O)N(R)₂. In some embodiments, R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is —C(O)NHR. In some embodiments, R^{w} is —C(O)NHR, wherein R of R^{w} is optionally substituted C₁₋₆ aliphatic. In some embodiments, R^{W} is —C(O)NHR, wherein R of R^{w} is C₁₋₆ aliphatic, optionally substituted with —CN.

In some embodiments, R¹ is optionally substituted C₁₋₆ aliphatic. In some embodiments, R^{w} is C₁₋₆ aliphatic, optionally substituted with —C(O)N(R^{o})₂. In some embodiments, R¹ is C₁₋₆ aliphatic, optionally substituted with —NR^{o}(O)N(R^{o})₂. In some embodiments, R^{w} is

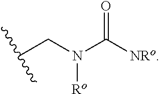

In some such embodiments, R^{o} is hydrogen or C₁₋₆ aliphatic. In some embodiments, R^{w} is C₁₋₆ aliphatic optionally substituted with —OR^{o}, wherein R^{o} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 5-6 membered saturated or partially unsaturated carbocyclic or heterocyclic ring having 1-2 heteroatoms independently selected from nitrogen, oxygen, and sulfur.

In some embodiments, R^{w} is an optionally substituted

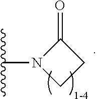

In some embodiments, R^{w} is an optionally substituted

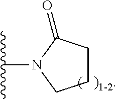

In some embodiments, R^{w} is an optionally substituted

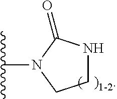

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{o})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{o})₂.

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{w} is optionally substituted pyridinyl, pyrimidinyl, pyridazinyl, or triazinyl. In some embodiments, R^{w} is an optionally substituted pyridinonyl, pyrazinonyl, or pyrimidinonyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In embodiments, R¹ is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{W} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w} is —C(S)R, —C(NR)R, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R—S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R¹ is —S(O)R, —S(O)₂R, —S(O)(NR)R—S(O)₂NR₂, —S(O)R, or —NRS(O)₂R. In some embodiments, R^{W} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R, —S(O)₂NR₂, —S(O)R, —C(S)R, —C(NR)R, —C(O)R, —C(O)OR, —C(O)NR₂, or —C(O)NROR. In some embodiments, R^{w} is —C(S)R, —C(NR)R, —C(O)R, —C(O)OR, or —C(O)NR₂.

In some embodiments, R^{w} is —C(O)R, wherein R is of R^{w} is optionally substituted C₁₋₆ aliphatic. In some embodiments, R^{w} is —C(O)R, wherein R is of R¹ is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R¹ is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OR, wherein R is of R^{w} is optionally substituted C₁₋₆ aliphatic. In some embodiments, R^{w} is —C(O)OR, wherein R is of R^{w} is optionally substituted phenyl. In some embodiments, R¹ is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NR₂, wherein each R is of R^{w} is independently hydrogen or an optionally substituted C₁₋₆ aliphatic. In some embodiments, R¹ is —C(O)NR₂, wherein each R is of R¹ is independently hydrogen or an optionally substituted phenyl. In some embodiments, R¹ is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(NR)R. In some embodiments, R^{w} is C(NR)R, wherein each R is of R^{w} is independently hydrogen or an optionally substituted C₁₋₆ aliphatic. In some embodiments, R^{w} is C(NR)R, wherein each R is of R^{w} is independently hydrogen or an optionally substituted phenyl. In some embodiments, R^{w} is C(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is C(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R¹ is —C(O)NR₂, wherein the two R groups of R^{w} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R¹ are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R¹ is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)R, wherein R is of R^{w} is optionally substituted C₁₋₆ aliphatic. In some embodiments, R^{w} is —OC(O)R, wherein R is of R is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R¹ is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R¹ is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R¹ is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{W} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)₂, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)R, wherein R is of R^{w} is optionally substituted C₁₋₆ aliphatic. In some embodiments, R^{w} is —S(O)R, wherein R is of R^{w} is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R^{w} is independently hydrogen or optionally substituted phenyl. In some embodiments, R^{w} is —S(O)(NR)R, wherein each R is of R is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R^{w} is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

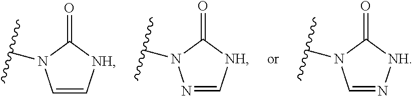 [SMILES: *n1cc[nH]c1=O ; *n1nc[nH]c1=O ; "or" ; *n1cn[nH]c1=O]

In some embodiments, R^{w} is:

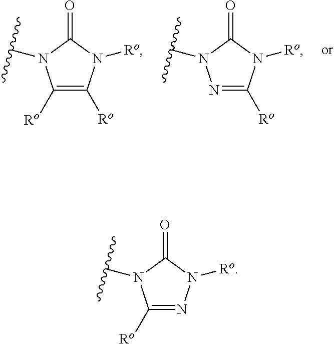

In some such embodiments, R^{o} is hydrogen or C₁₋₆ aliphatic. In some embodiments, R^{w} is:

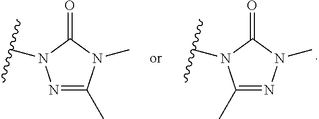

In some embodiments, R^{w} is an optionally substituted:

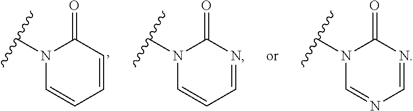 [SMILES: *n1ccccc1=O ; "," ; *n1cccnc1=O ; "or" ; *n1cncnc1=O]

In some embodiments, R^{w} is:

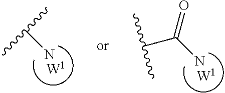

wherein Ring W¹ is as defined above and described herein.

In some embodiments, R^{w} is:

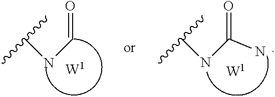

In some embodiments, R^{w} is:

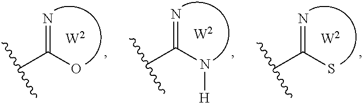

wherein Ring W² is as defined above and described herein.

In some embodiments, R^{w} is —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr,

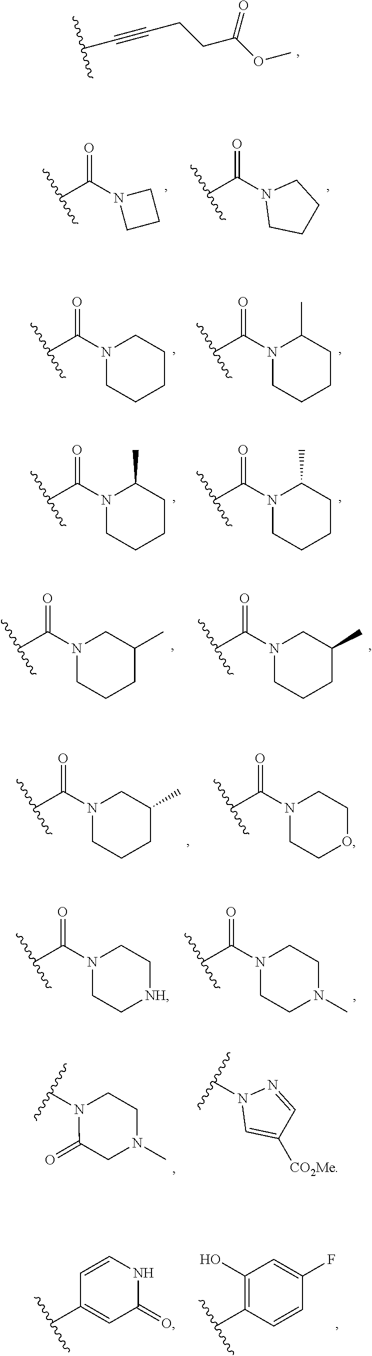

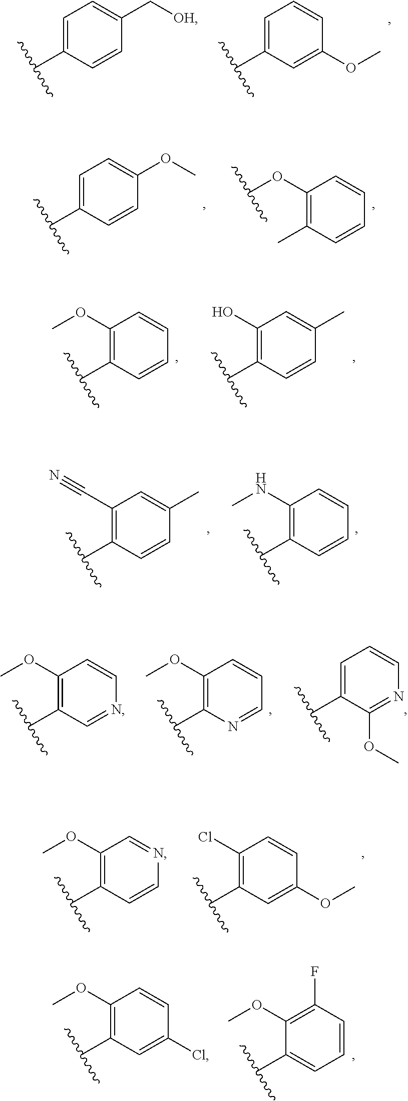

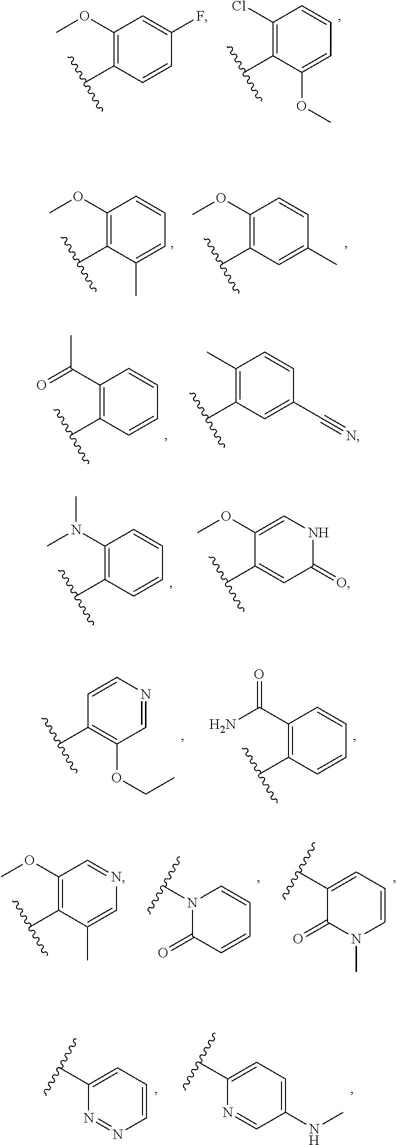

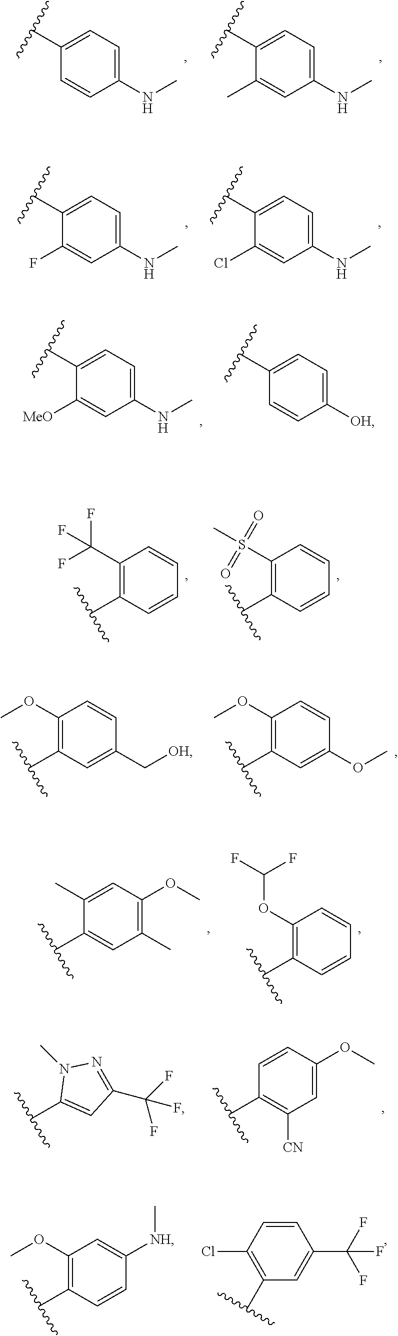

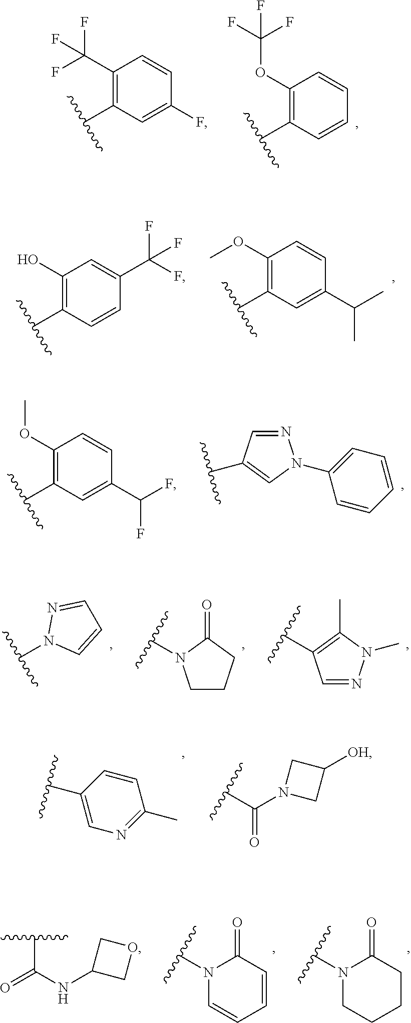

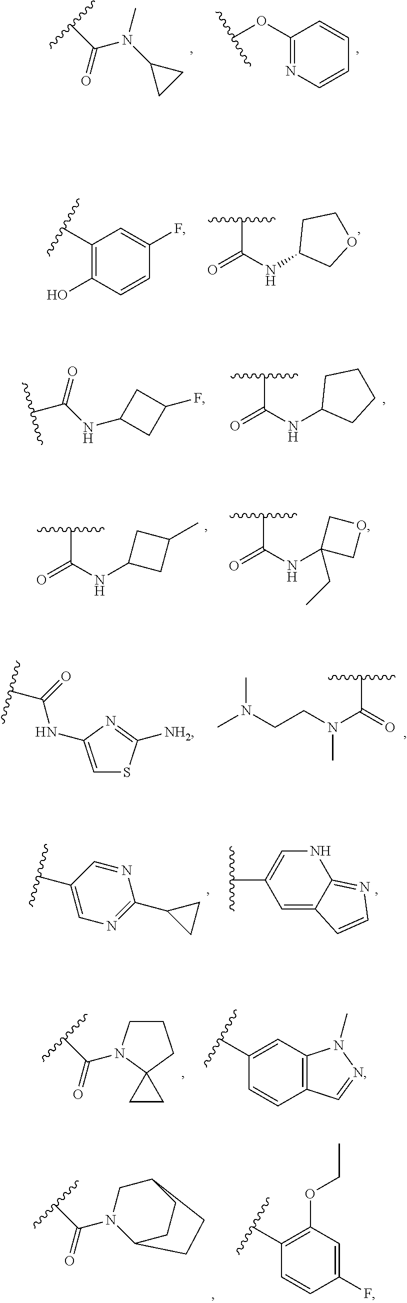

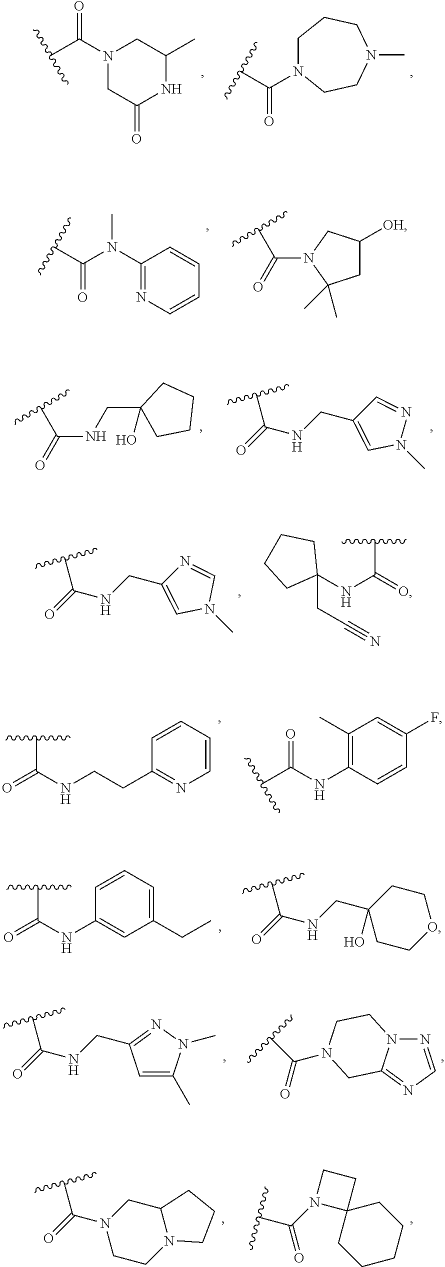

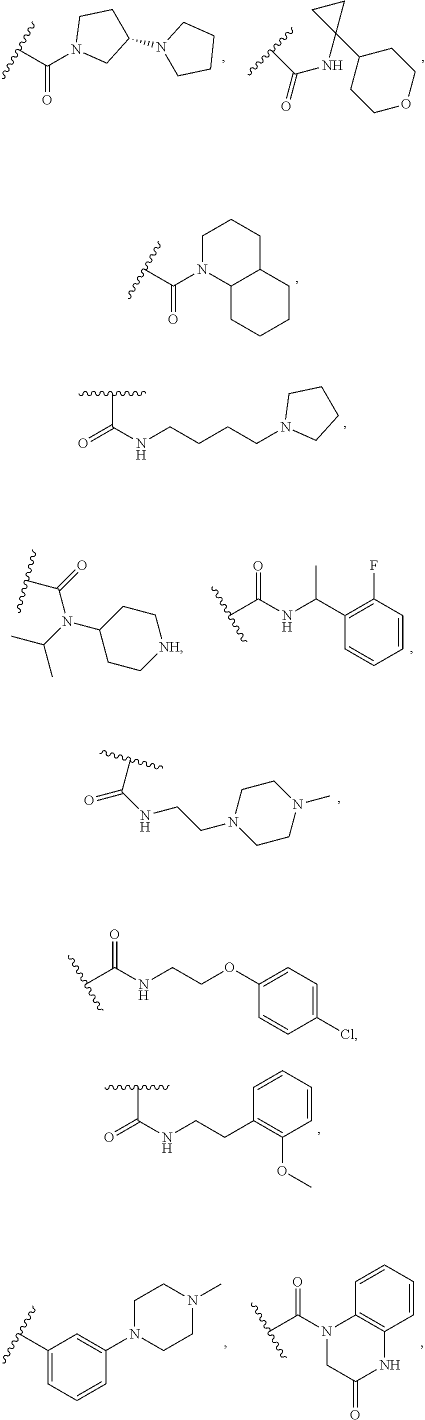

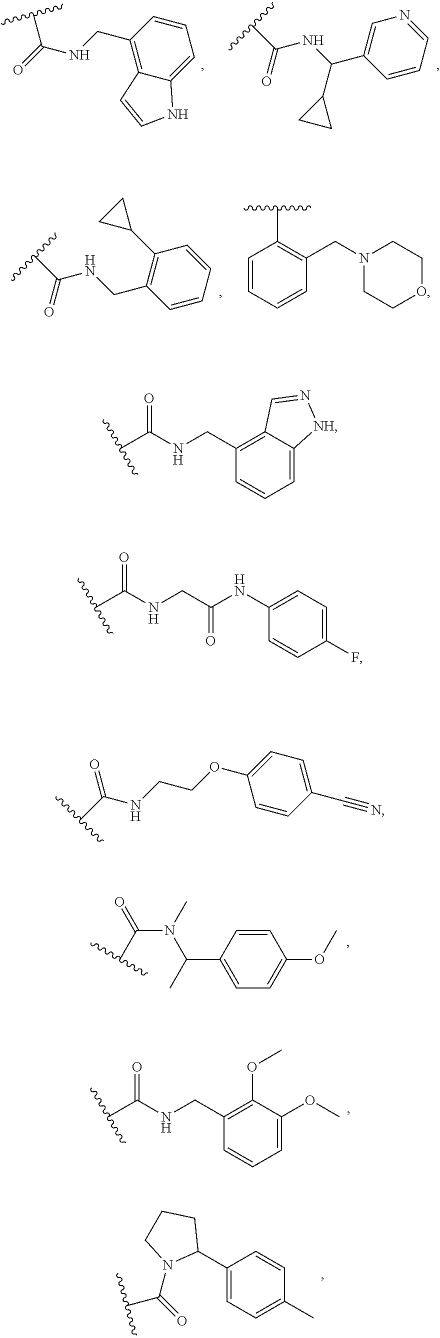

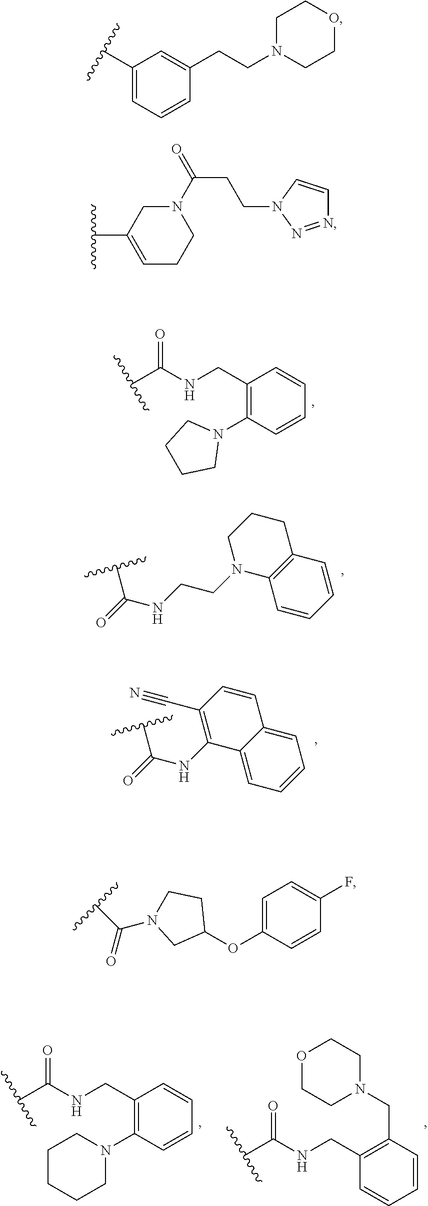

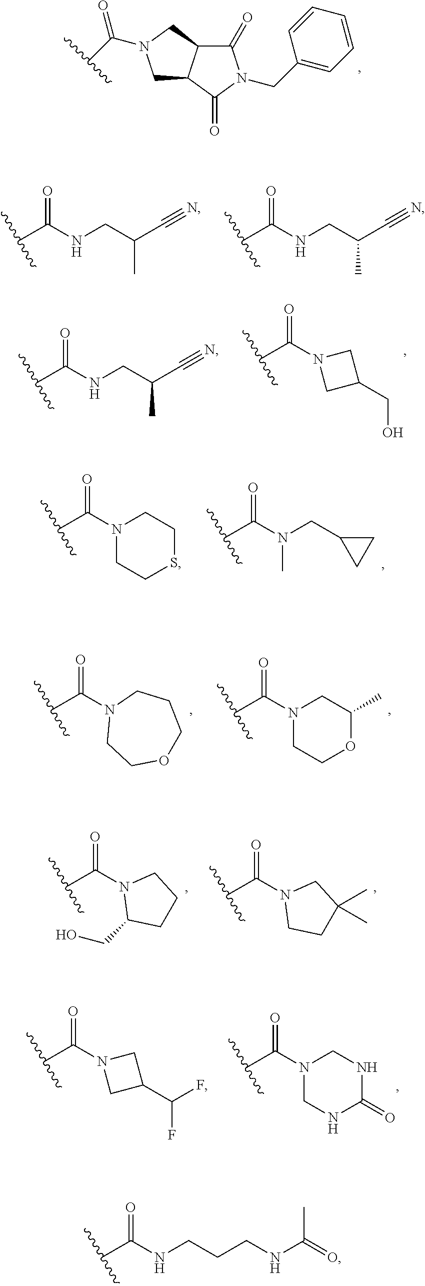

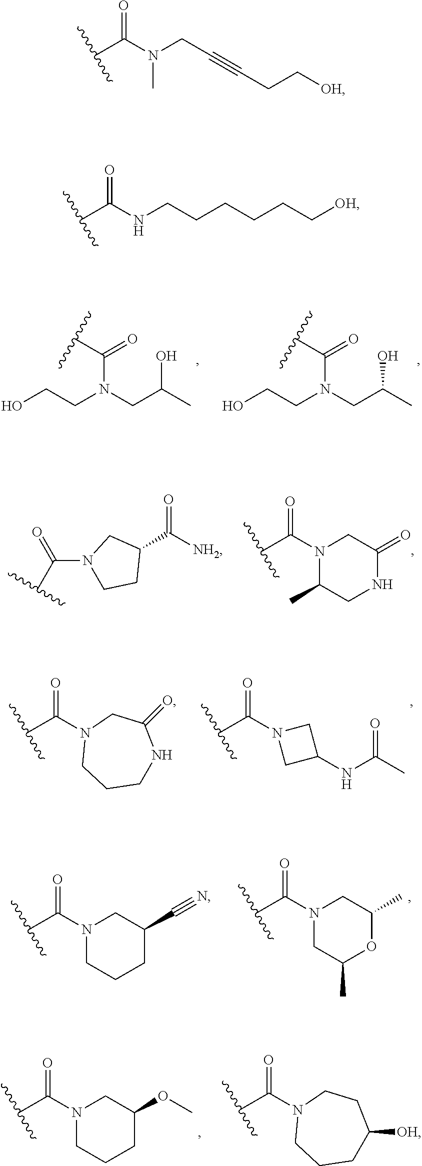

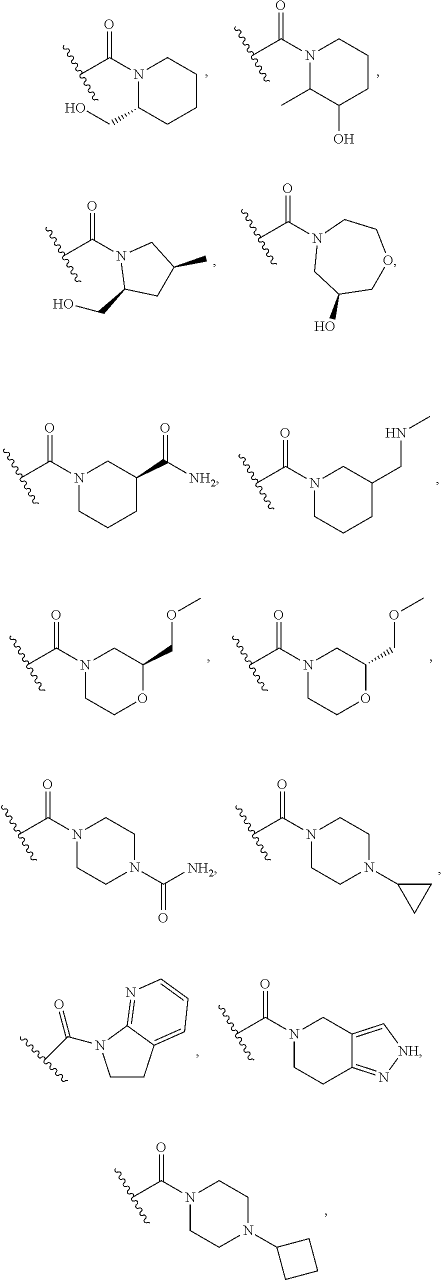

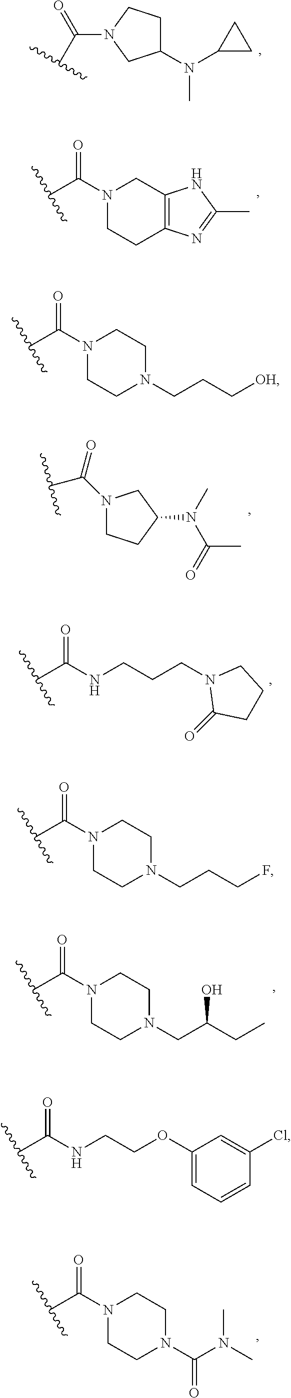

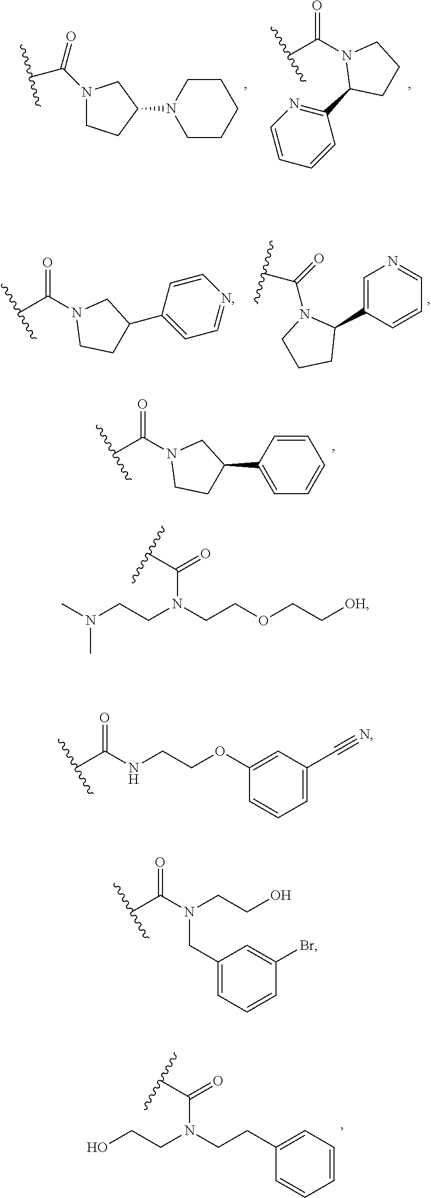

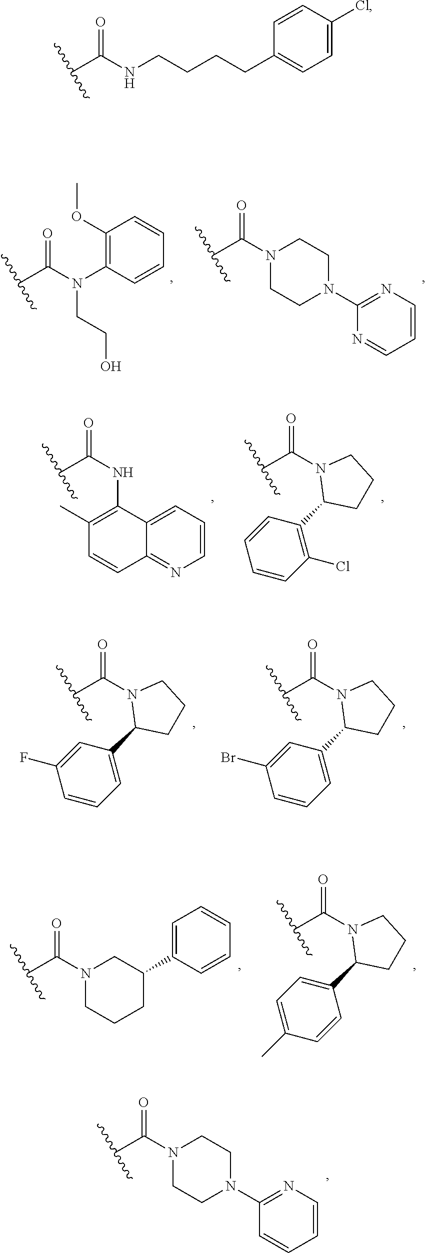

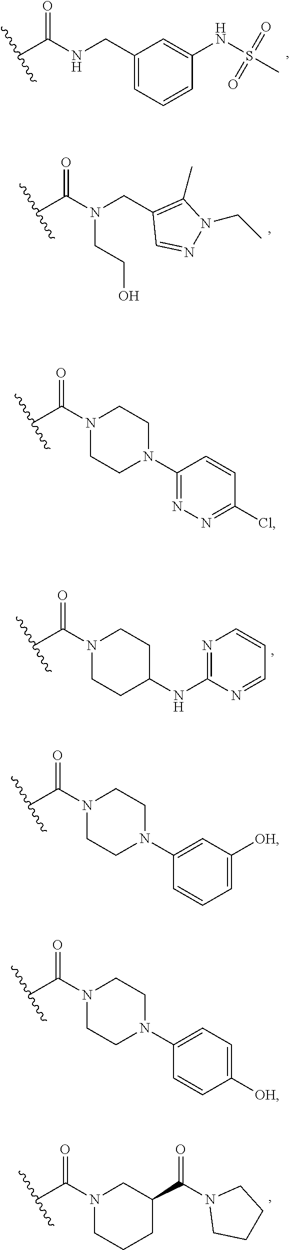

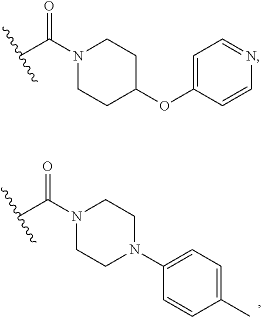 [SMILES: *C(=O)N1CCC(Oc2ccncc2)CC1 ; *C(=O)N1CCN(c2ccc(C)cc2)CC1]

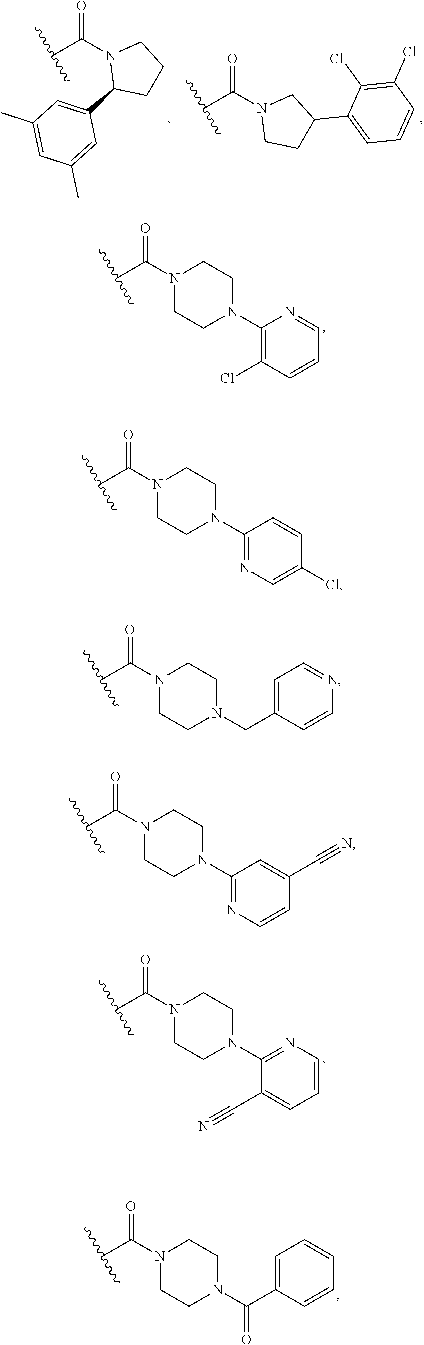

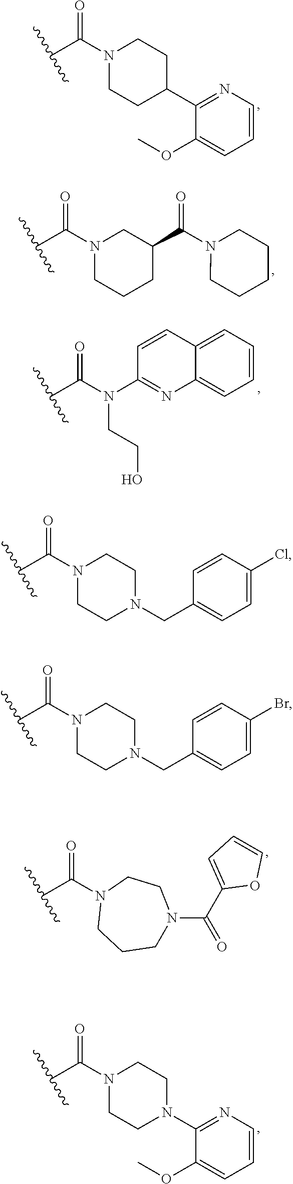

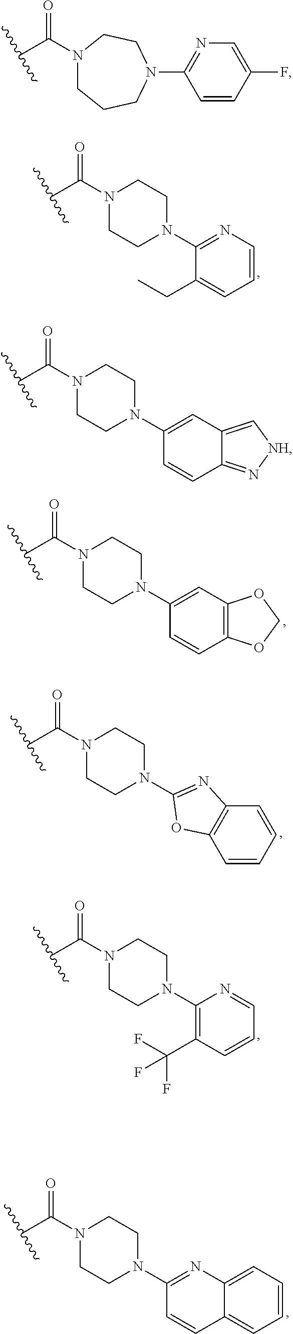

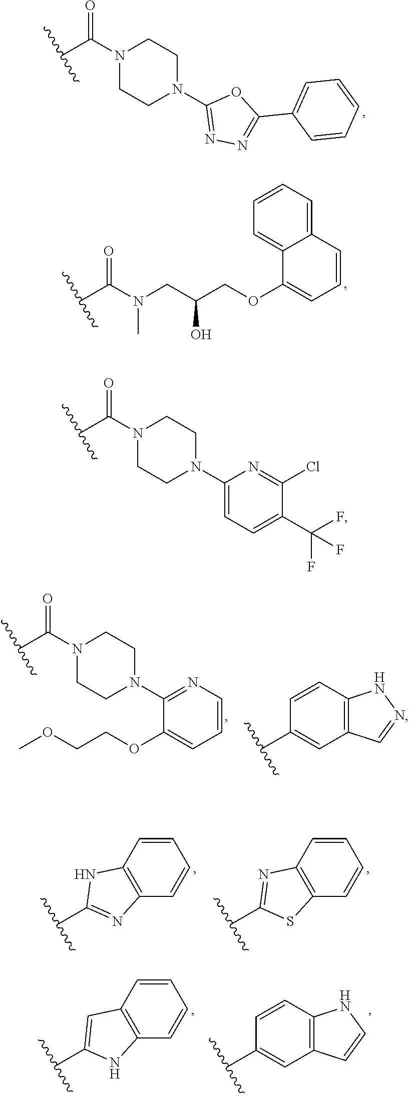

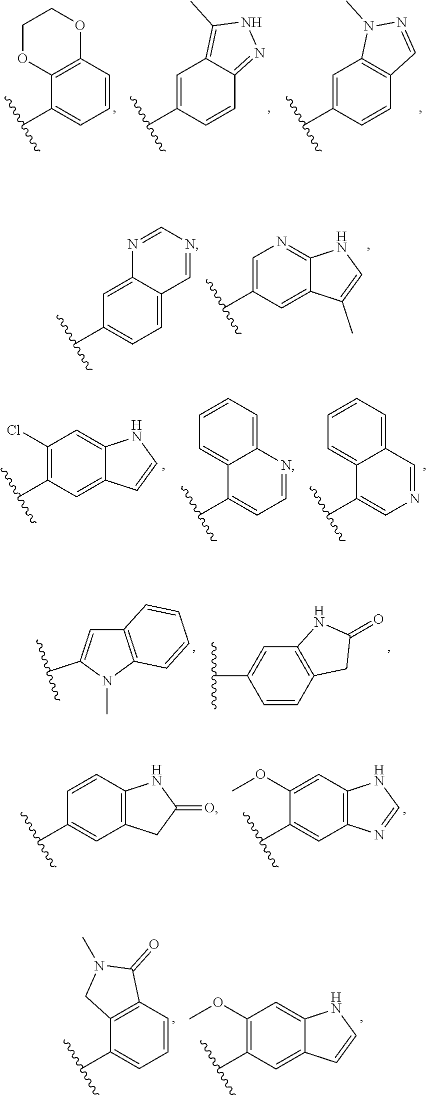

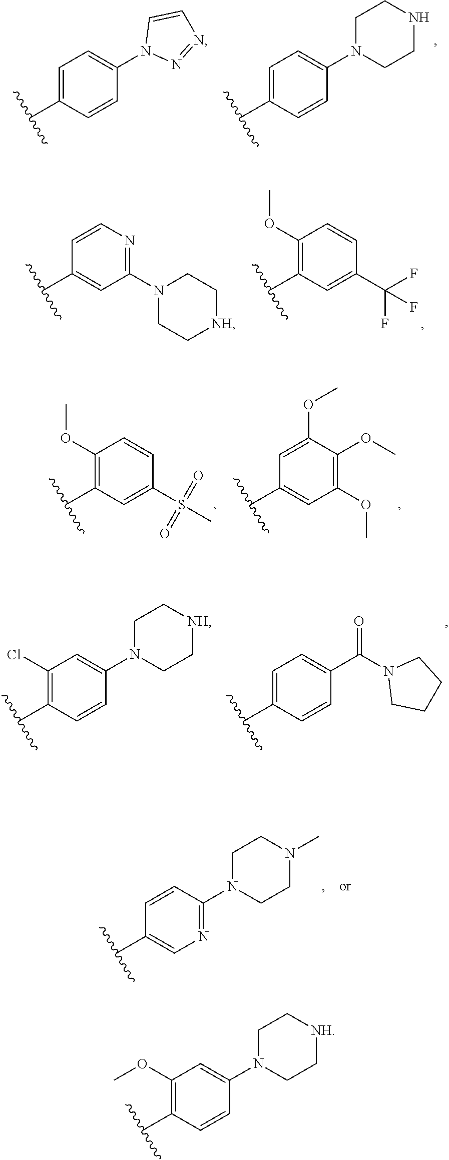

In some embodiments, R is

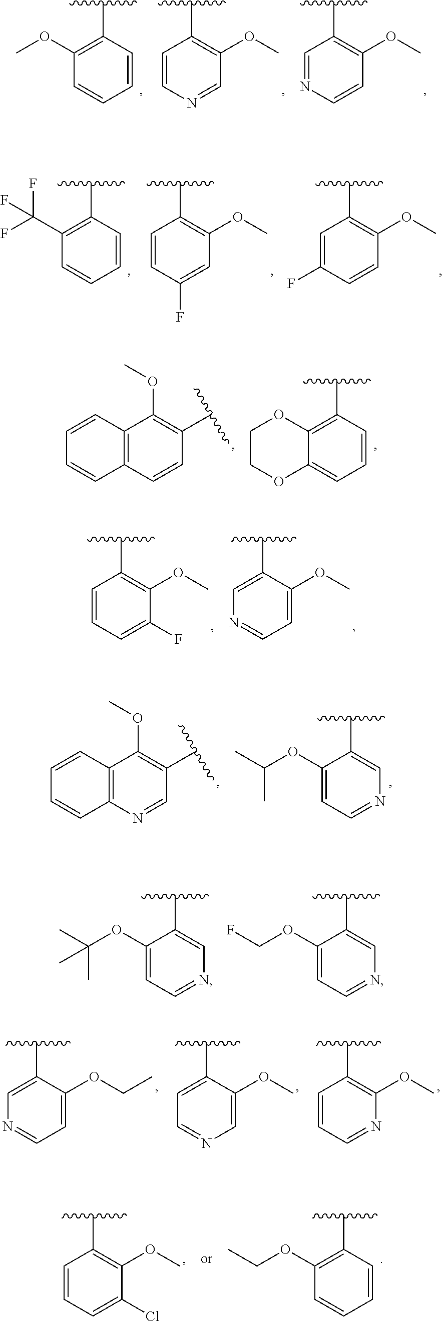

In some embodiments, R^{w} is —S(O)₂NH₂, —S(O)₂N(CH₃)₂, —S(O)(NH)CH₃,

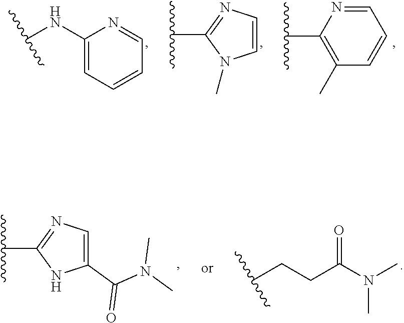

In some embodiments, the present invention provides methods of degrading STAT6, or a mutant thereof, or methods of treating a STAT6-mediated disease, disorder, or condition comprising administering a compound of any one of the following formulae:

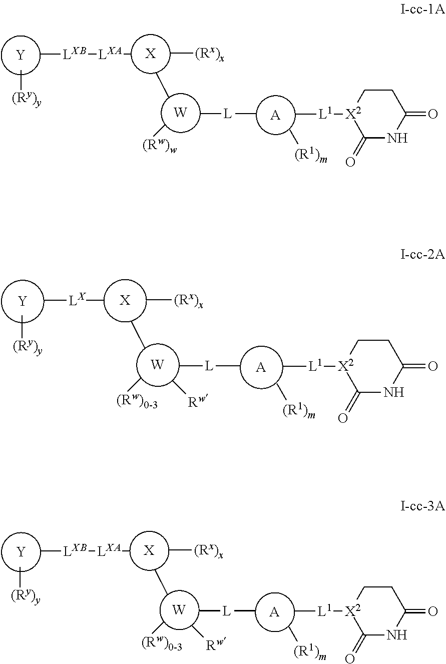

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

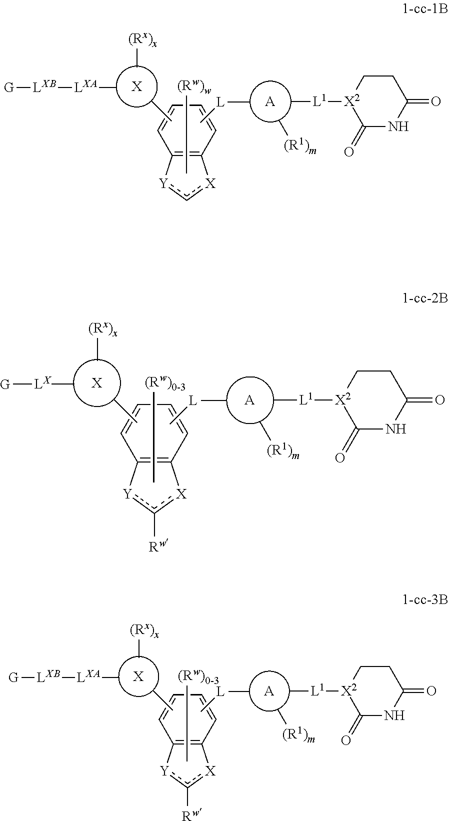

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

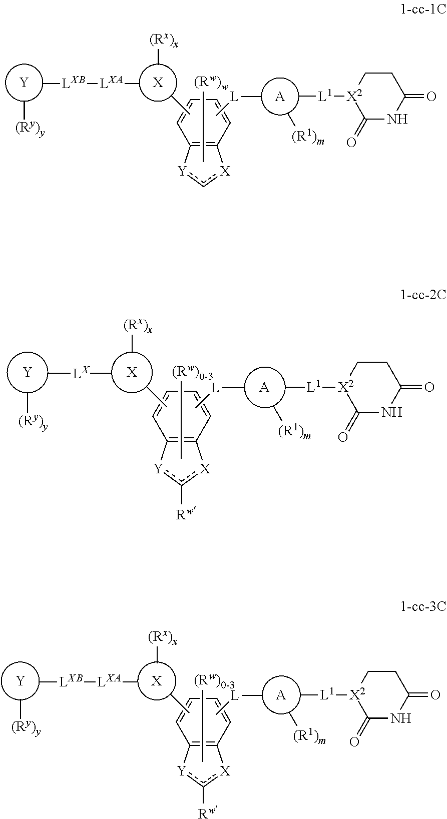

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

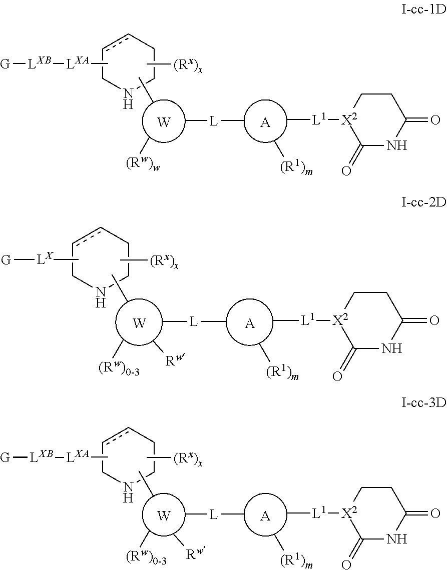

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

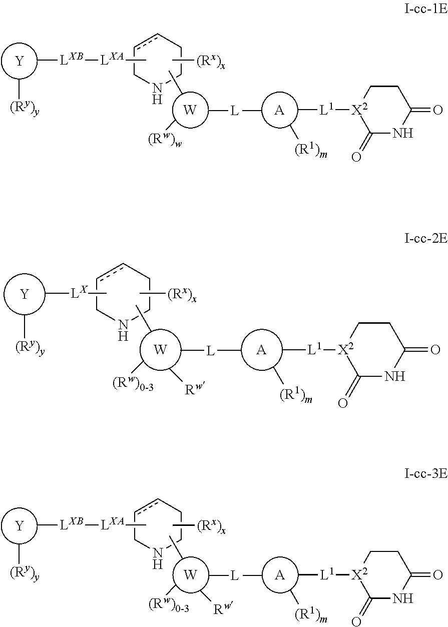

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

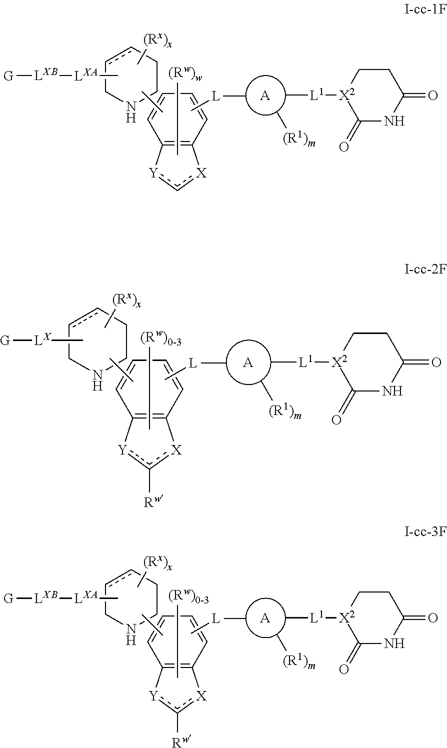

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

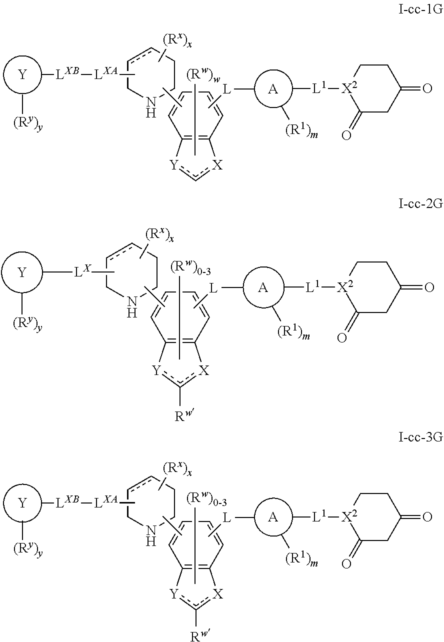

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

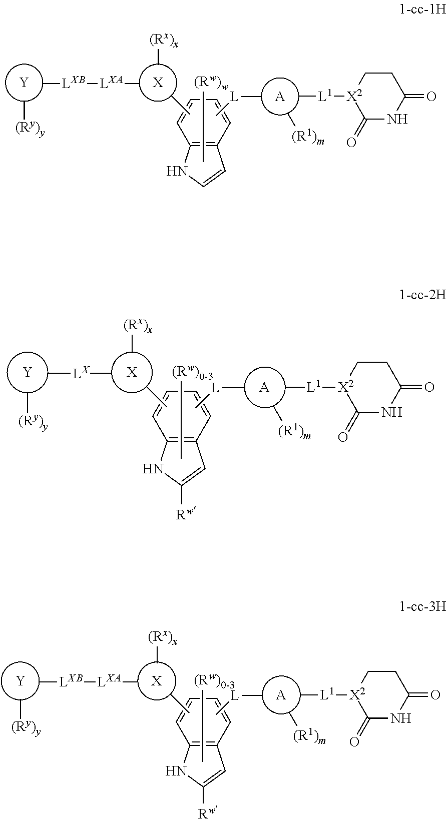

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

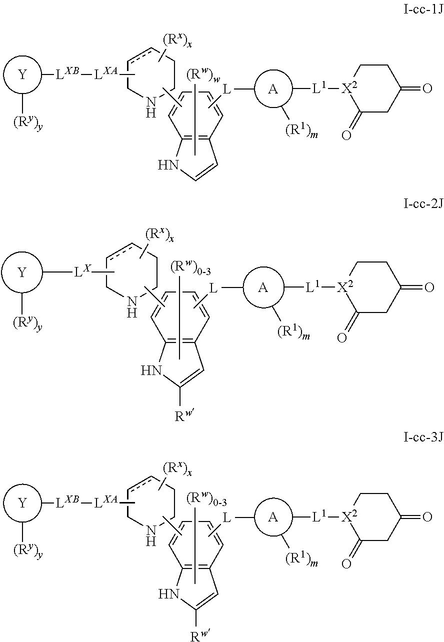

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A, I-cc-1B. I-cc-2B, I-cc-3B, I-cc-1C, I-cc-2C, I-cc-3C, I-cc-1D, I-cc-2D, I-cc-3D, I-cc-1E, I-cc-2E, I-cc-3E, I-cc-1F, I-cc-2F, I-cc-3F, I-cc-1G, I-cc-2G, I-cc-3G, I-cc-1H, I-cc-2H, I-cc-3H, I-cc-1J, I-cc-2J, or I-cc-3J, the structure

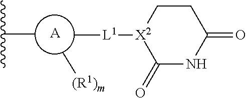

is of any one of formula I-aa-10′, I-aa-11′, I-aa-12′, I-aa-13′, or I-aa-14′:

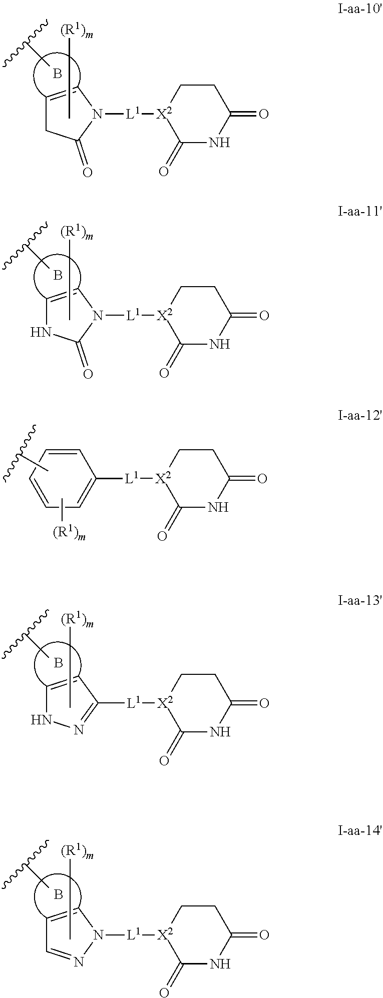

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A. I-cc-1B, I-cc-2B, I-cc-3B, I-cc-1C, I-cc-2C, I-cc-3C, I-cc-1D, I-cc-2D, I-cc-3D, I-cc-1E, I-cc-2E, I-cc-3E, I-cc-1F, I-cc-2F, I-cc-3F, I-cc-1G, I-cc-2G, I-cc-3G, I-cc-1H, I-cc-2H, I-cc-3H, I-cc-1J, I-cc-2J, or I-cc-3J, the structure

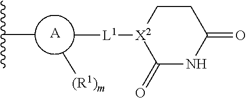

is of any one of formula I-aa-10a′, I-aa-11a′, I-aa-12a′, I-aa-12b′, I-aa-13a′, or I-aa-14a′:

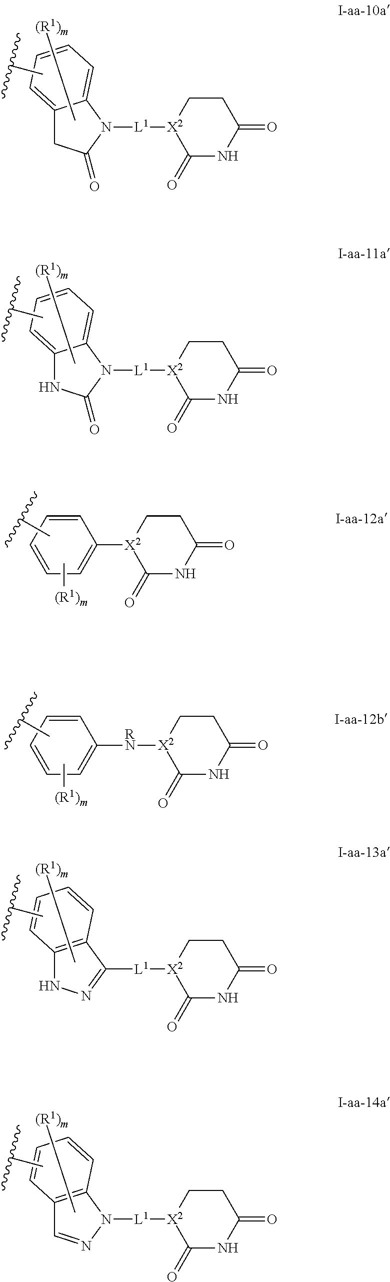

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

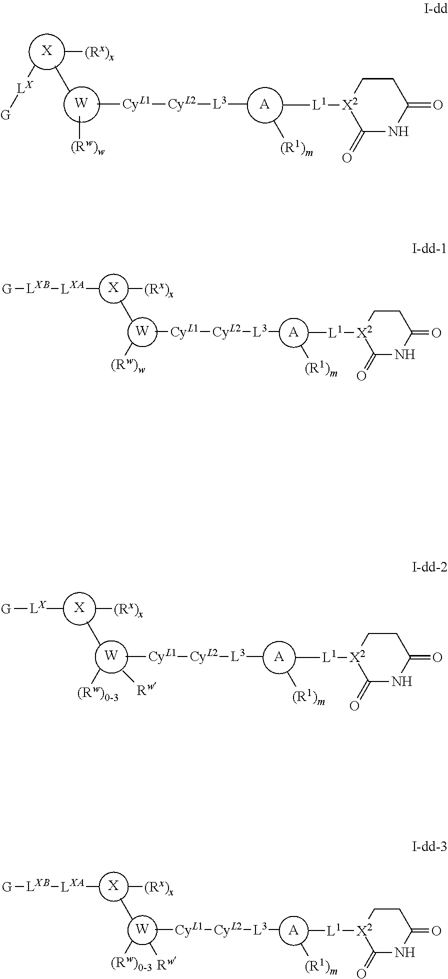

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

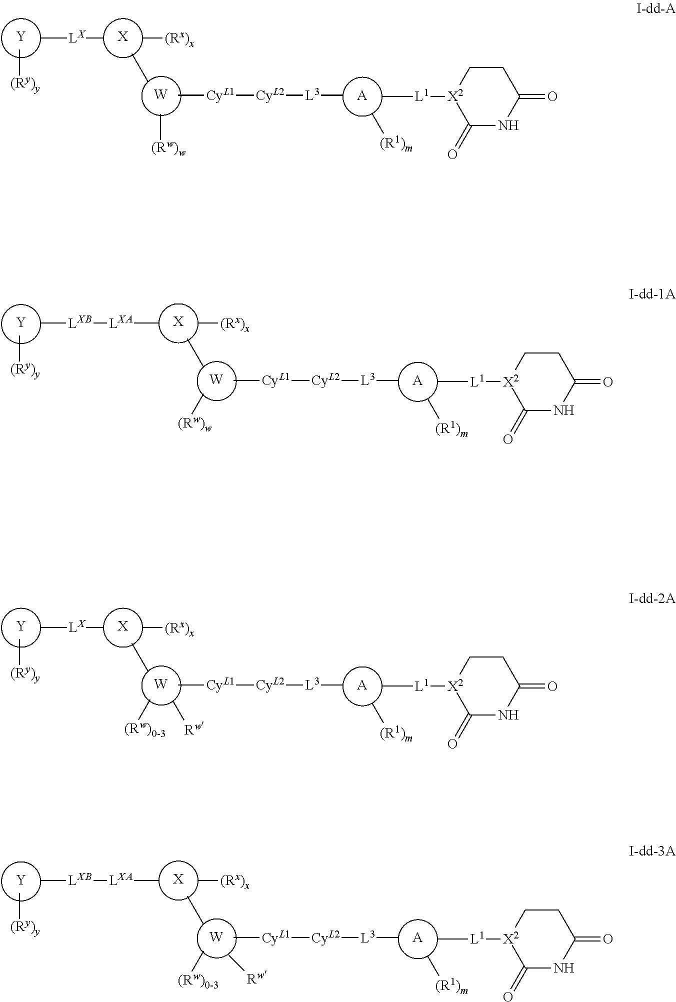

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

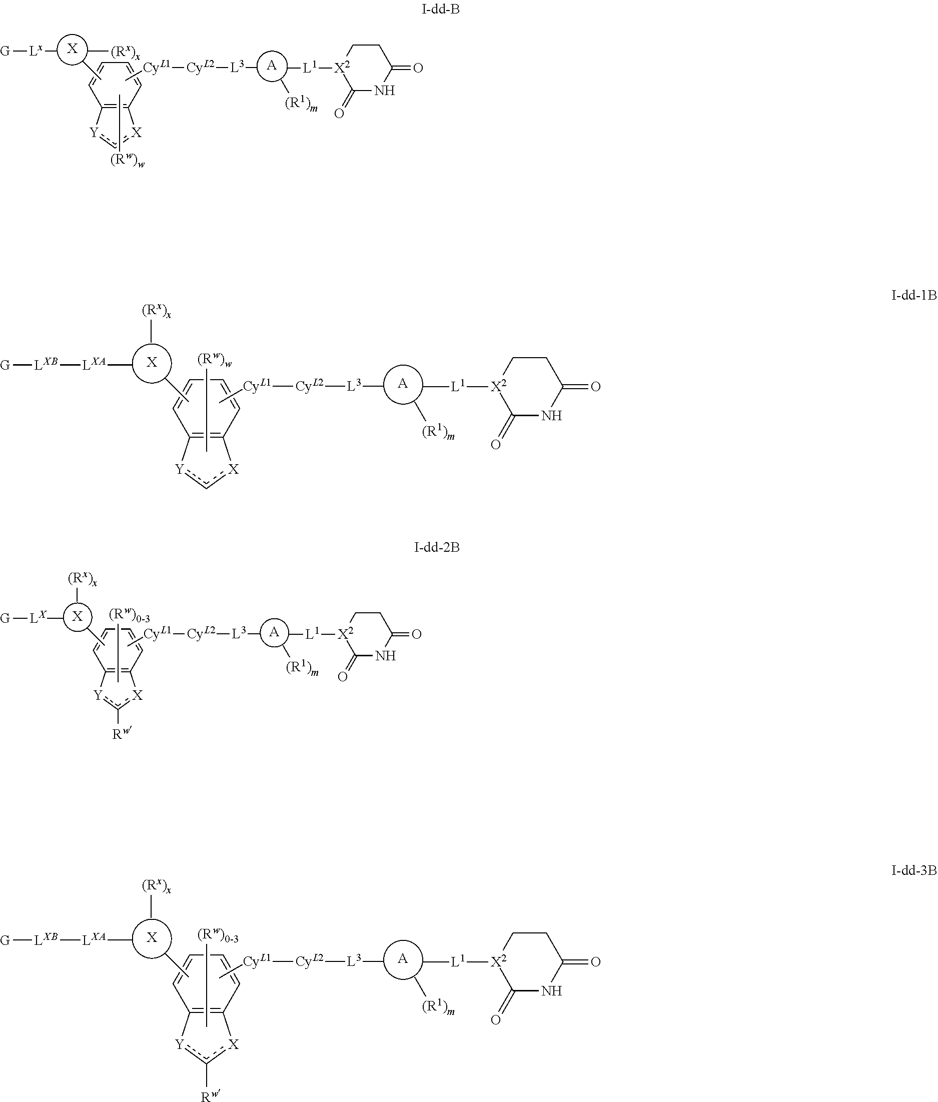

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

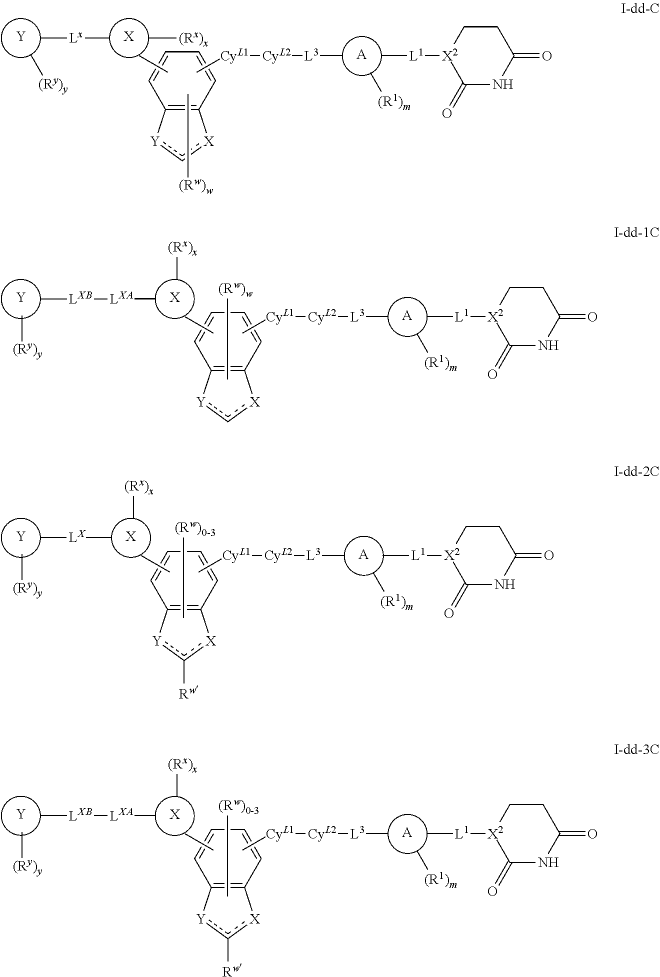

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

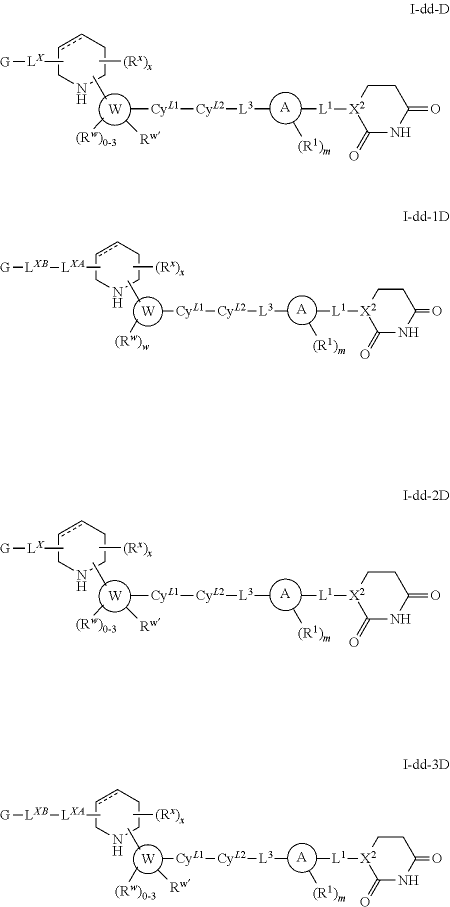

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

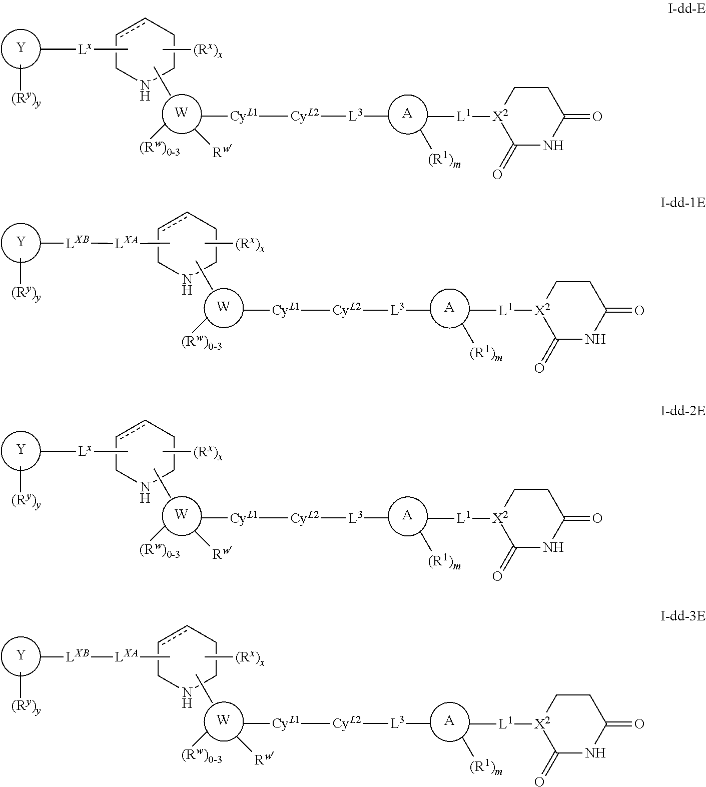

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

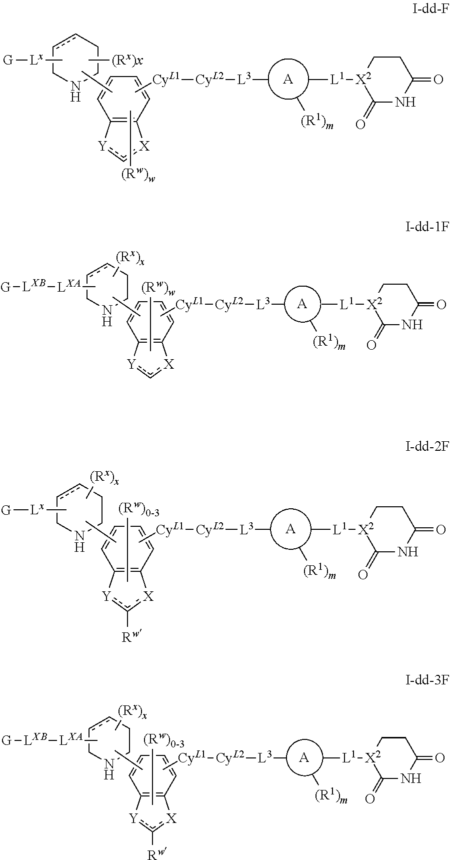

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

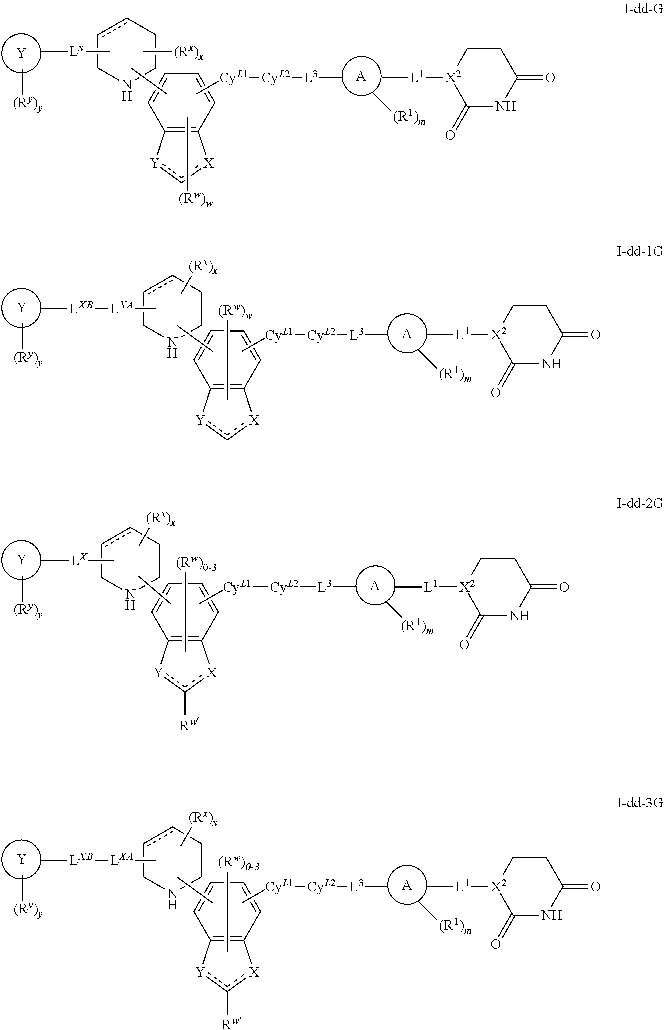

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

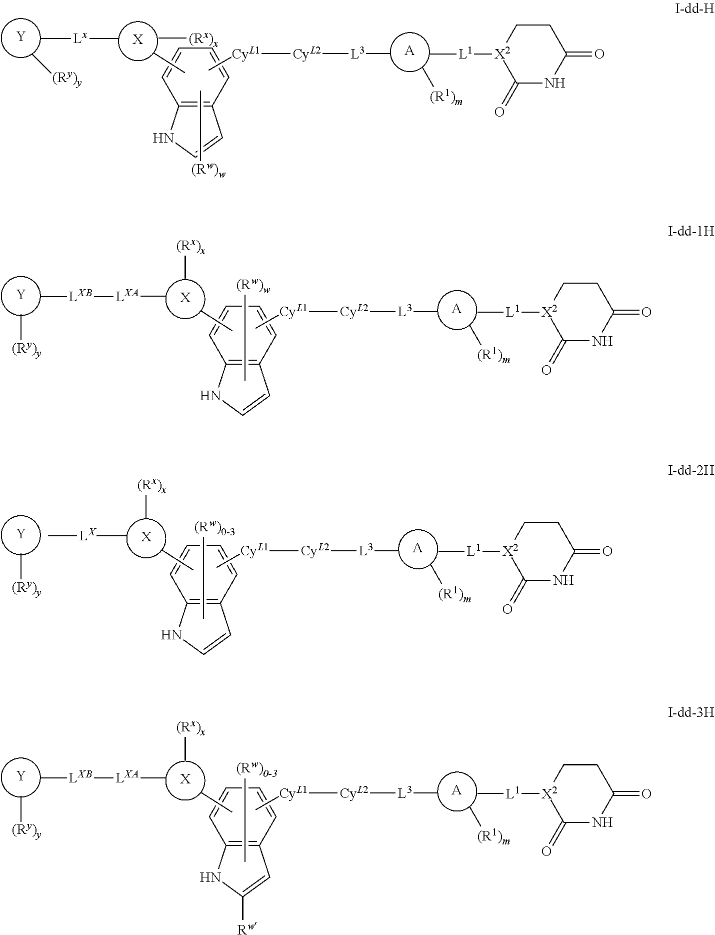

or a pharmaceutically acceptable salt thereof, wherein each variable is as defined above and described herein both individually and in combination.

In some embodiments, the present invention provides methods of degrading STAT6, or a mutant thereof, or methods of treating a STAT6-mediated disease, disorder, or condition comprising administering a compound of any one of the following formulae:

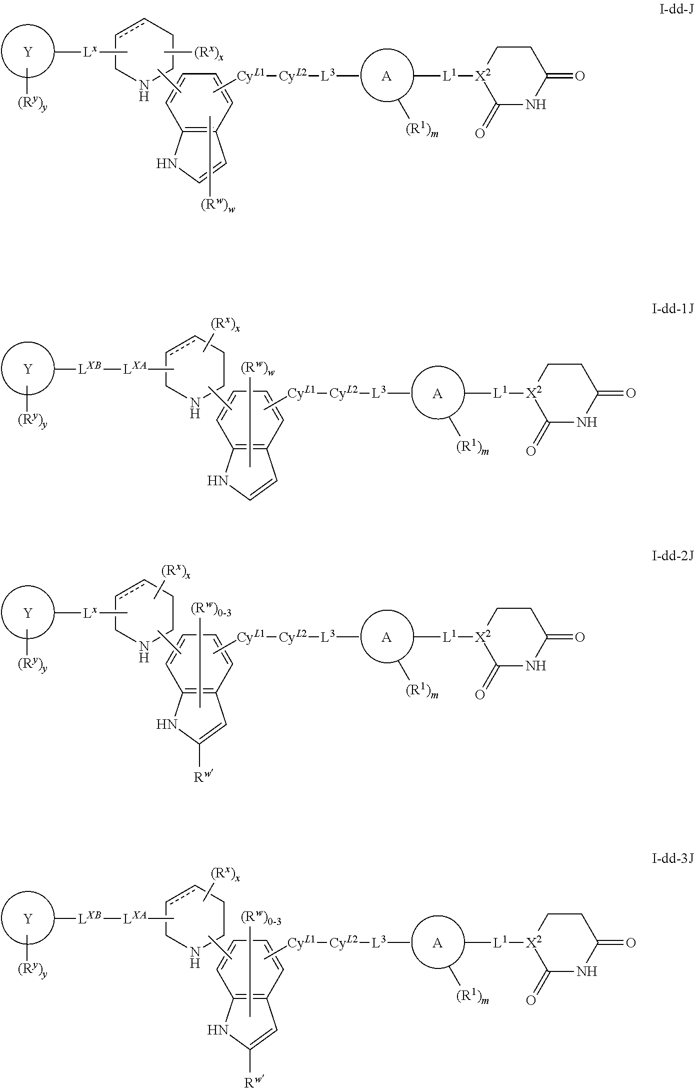

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B, I-dd-C, I-dd-1C, I-dd-2C, I-dd-3C, I-dd-D. I-dd-1D, I-dd-2D, I-dd-3D, I-dd-E, I-dd-1E, I-dd-2E. I-dd-3E. I-dd-F. I-dd-1F, I-dd-2F, I-dd-3F, I-dd-G, I-dd-1G, I-dd-2G, I-dd-3G, I-dd-H, I-dd-1H, I-dd-2H, I-dd-3H, I-dd-J, I-dd-1J, I-dd-2J, or I-dd-3J, the structure

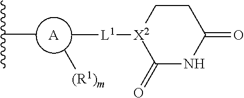

is of any one of formula I-aa-10′, I-aa-11′, I-aa-12′, I-aa-13′, or I-aa-14′:

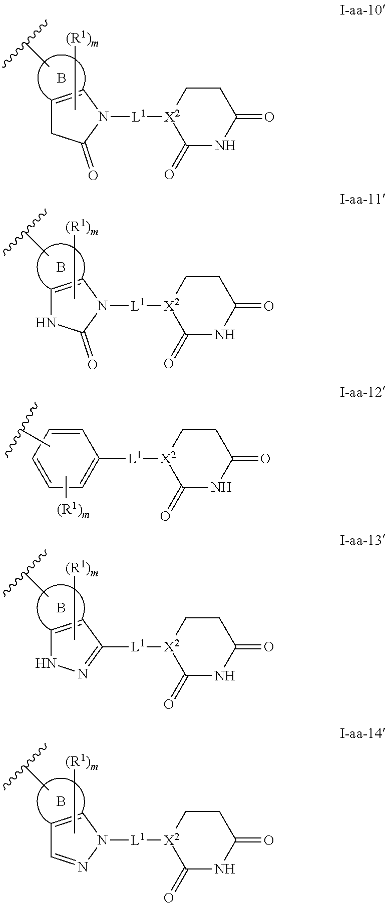

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B, I-dd-C, I-dd-1C, I-dd-2C, I-dd-3C, I-dd-D, I-dd-1D, I-dd-2D, I-dd-3D, I-dd-E, I-dd-1E, I-dd-2E, I-dd-3E, I-dd-F, I-dd-1F, I-dd-2F, I-dd-3F, I-dd-G, I-dd-1G, I-dd-2G, I-dd-3G, I-dd-H, I-dd-1H, I-dd-2H, I-dd-3H, I-dd-J, I-dd-1J, I-dd-2J, or I-dd-3J, the structure

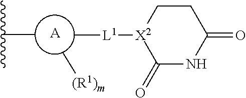

is of any one of formula I-aa-10a′, I-aa-11a′, I-aa-12a′, I-aa-12b′, I-aa-13a′, or I-aa-14a′:

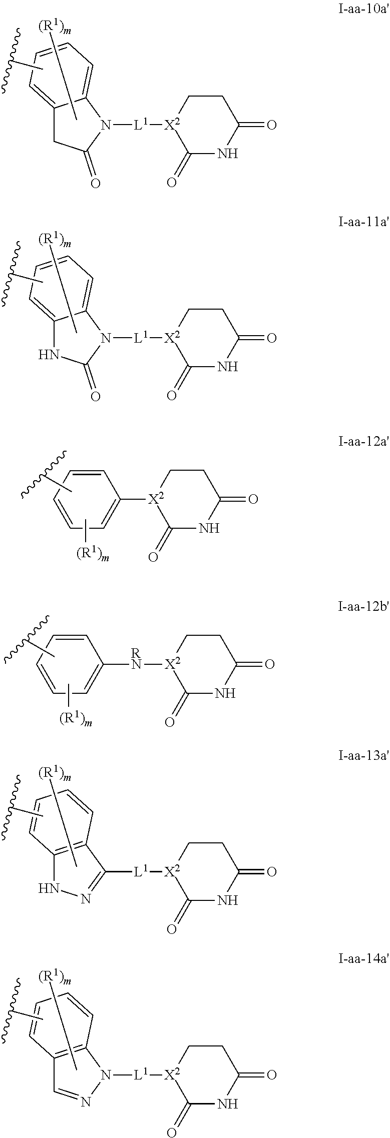

or a pharmaceutically acceptable salt thereof, wherein each variable is as defined above and described herein both individually and in combination.

Exemplary compounds for use in provided methods of the invention are set forth in Table 1A, below. It is understood that certain compounds herein may contain arbitrarily assigned stereochemistry or be derived from an intermediate with arbitrarily assigned stereochemistry. Unless otherwise stated, stereochemistry for compounds herein has been assigned arbitrarily, and it will be understood that any compound with arbitrarily assigned stereochemistry or produced from an intermediate with arbitrarily assigned stereochemistry may be depicted herein as a certain stereoisomer, but such compound may be the other stereoisomer (i.e., enantiomer or diastereomer). As shown in Table 1A, column “EO”, for instances where a pair of stereoisomers were produced and the final compounds separated, the order of elution is provided: the first eluting isomer is indicated by “E1” and the second elution isomer is indicated by “E2”. As shown in Table 1A, column “EO”, for instances where a pair of stereoisomers were produced, but a pair of intermediate stereoisomers were separated and further modified to arrive at the final compounds, the order of elution of the intermediate compound is provided: the first eluting isomer of the intermediate compound is indicated by “I1”, and the second eluting isomer of the intermediate compound is indicted by “I2.”

TABLE 1A
Exemplary Compounds

| I- | Structure |
|---|---|
| 61 | 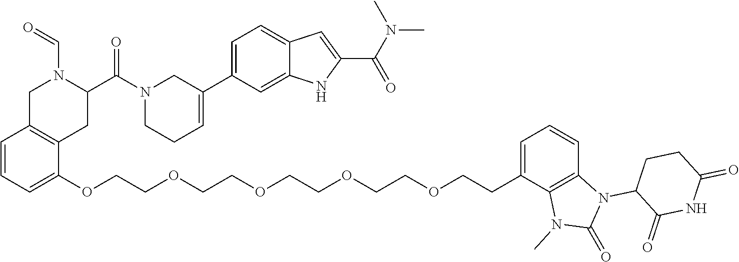 [SMILES: CN(C)C(=O)c1cc2ccc(C3=CCCN(C(=O)C4Cc5c(cccc5OCCOCCOCCOCCOCCc5cccc6c5n(C)c(=O)n6C5CCC(=O)NC5=O)CN4C=O)C3)cc2[nH]1] |
| 98 | 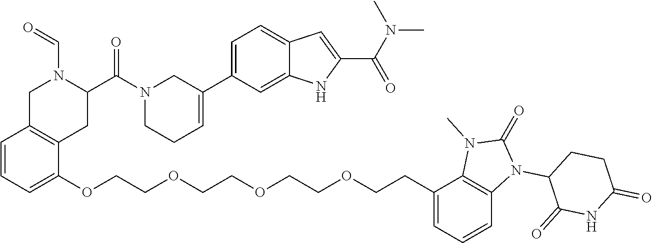 [SMILES: CN(C)C(=O)c1cc2ccc(C3=CCCN(C(=O)C4Cc5c(cccc5OCCOCCOCCOCCc5cccc6c5n(C)c(=O)n6C5CCC(=O)NC5=O)CN4C=O)C3)cc2[nH]1] |
| 105 | 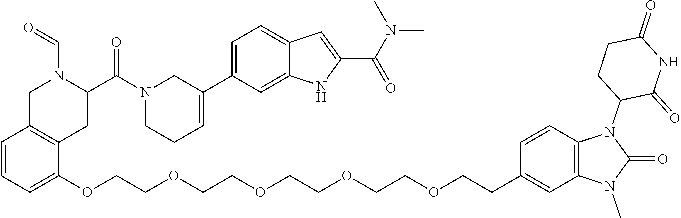 [SMILES: CN(C)C(=O)c1cc2ccc(C3=CCCN(C(=O)C4Cc5c(cccc5OCCOCCOCCOCCOCCc5ccc6c(c5)n(C)c(=O)n6C5CCC(=O)NC5=O)CN4C=O)C3)cc2[nH]1] |
| 1270 | 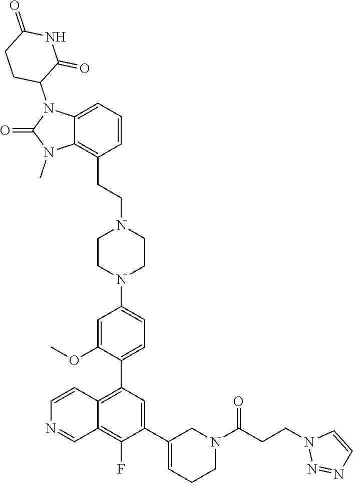 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2cnccc12] |
| 1271 | 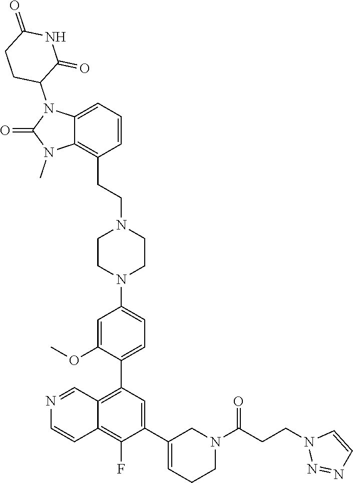 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2ccncc12] |
| 1272 | 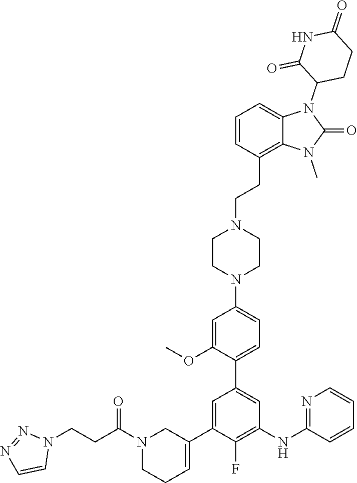 [SMILES: COc1cc(N2CCN(CCc3cccc4c3n(C)c(=O)n4C3CCC(=O)NC3=O)CC2)ccc1-c1cc(Nc2ccccn2)c(F)c(C2=CCCN(C(=O)CCn3ccnn3)C2)c1] |
| 1282 | 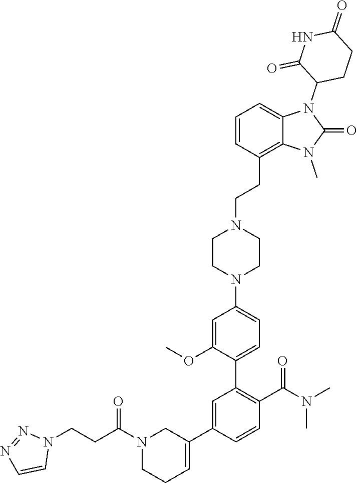 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1C(=O)N(C)C] |
| 1292 | 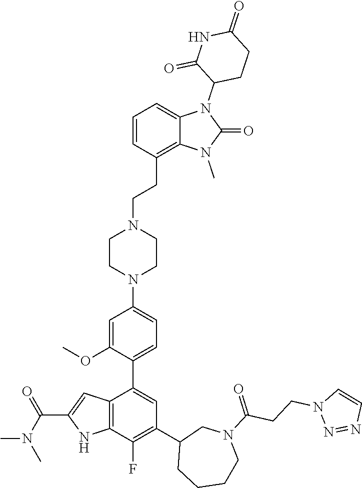 [SMILES: COc1cc(N2CCN(CCc3cccc4c3n(C)c(=O)n4C3CCC(=O)NC3=O)CC2)ccc1-c1cc(C2CCCCN(C(=O)CCn3ccnn3)C2)c(F)c2[nH]c(C(=O)N(C)C)cc12] |
| 1293 | 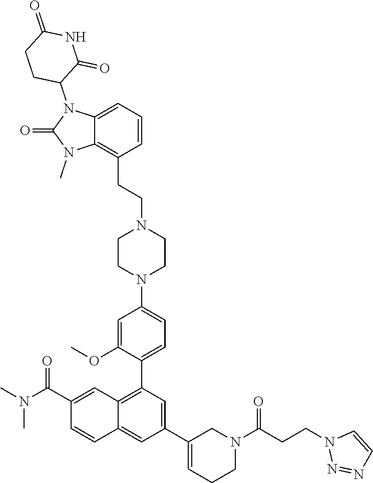 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccc(C(=O)N(C)C)cc12] |
| 1302 | 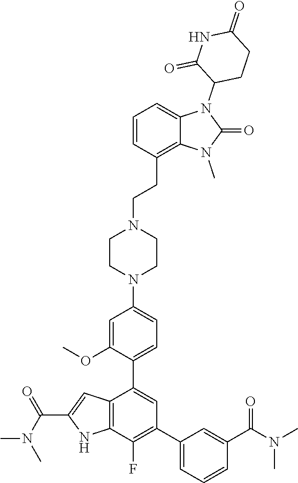 |
| 1309 | 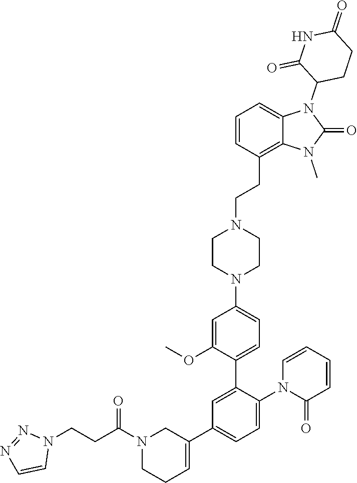 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-n1ccccc1=O] |
| 1323 | 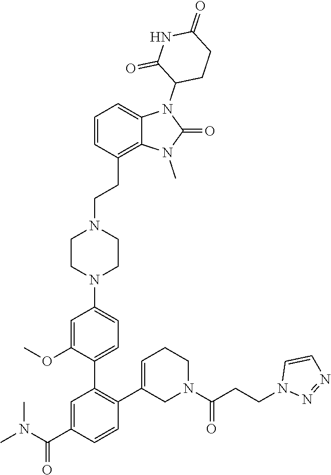 [SMILES: COc1cc(N2CCN(CCc3cccc4c3n(C)c(=O)n4C3CCC(=O)NC3=O)CC2)ccc1-c1cc(C(=O)N(C)C)ccc1C1=CCCN(C(=O)CCn2ccnn2)C1] |
| 1324 | 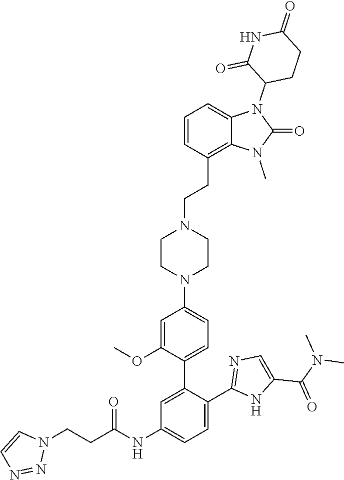 [SMILES: COc1cc(N2CCN(CCc3cccc4c3n(C)c(=O)n4C3CCC(=O)NC3=O)CC2)ccc1-c1cc(NC(=O)CCn2ccnn2)ccc1-c1ncc(C(=O)N(C)C)[nH]1] |
| 1325 | 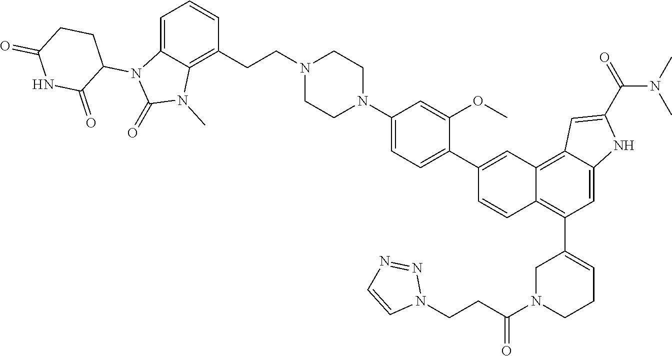 [SMILES: COc1cc(N2CCN(CCc3cccc4c3n(C)c(=O)n4C3CCC(=O)NC3=O)CC2)ccc1-c1ccc2c(C3=CCCN(C(=O)CCn4ccnn4)C3)cc3[nH]c(C(=O)N(C)C)cc3c2c1] |
| 1326 | 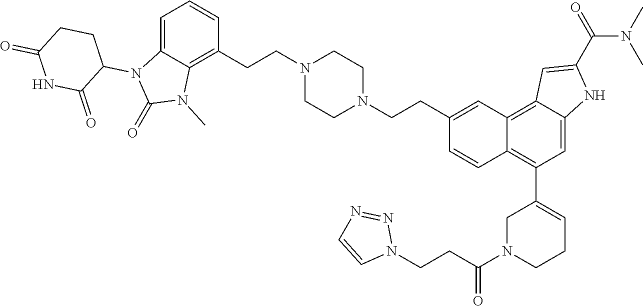 [SMILES: CN(C)C(=O)c1cc2c(cc(C3=CCCN(C(=O)CCn4ccnn4)C3)c3ccc(CCN4CCN(CCc5cccc6c5n(C)c(=O)n6C5CCC(=O)NC5=O)CC4)cc32)[nH]1] |
| 1332 | 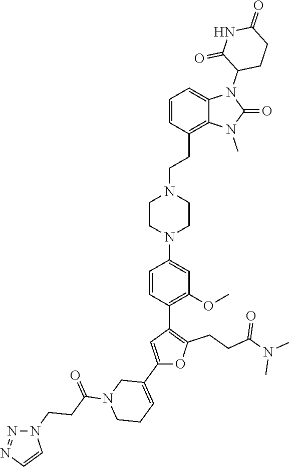 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)oc1CCC(=O)N(C)C] |
| 1333 | 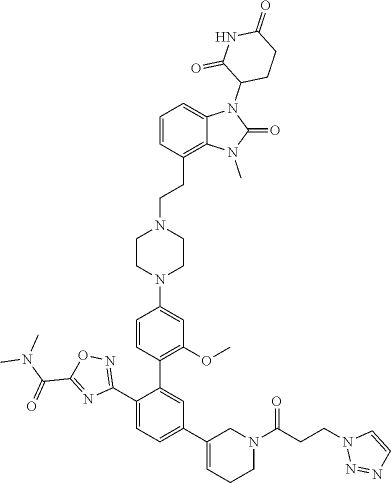 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-c1noc(C(=O)N(C)C)n1] |
| 1334 | 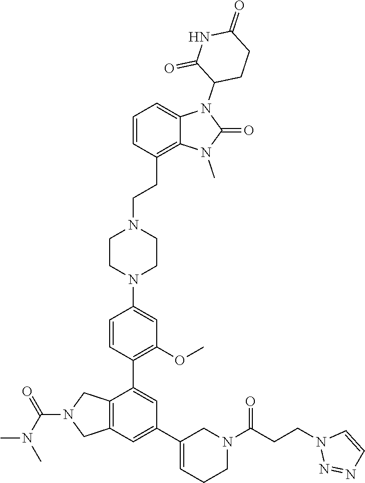 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1CN(C(=O)N(C)C)C2] |
| 1336 | 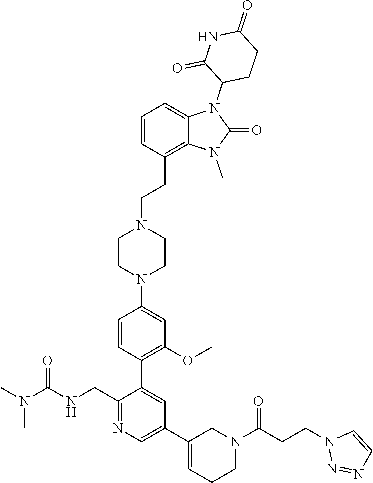 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cnc1CNC(=O)N(C)C] |
| 1338 | 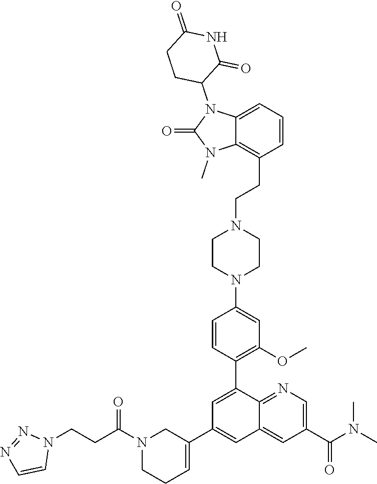 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2cc(C(=O)N(C)C)cnc12] |
| 1340 | 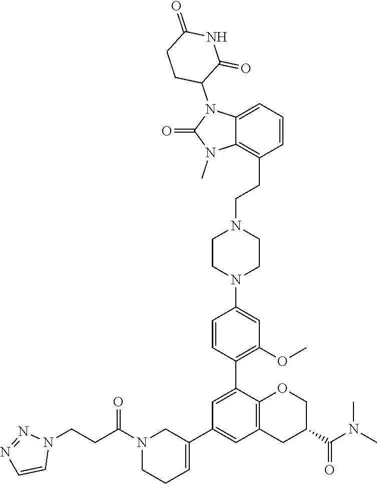 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1OC[C@H](C(=O)N(C)C)C2] |
| 1342 | 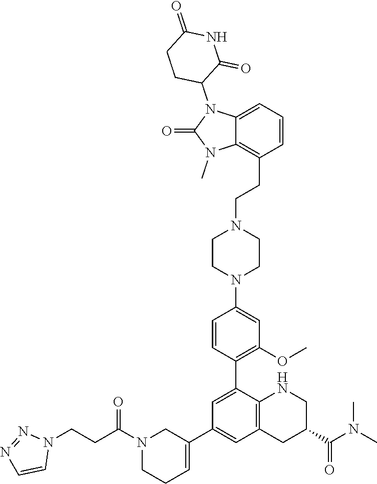 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1NC[C@H](C(=O)N(C)C)C2] |
| 1401 | 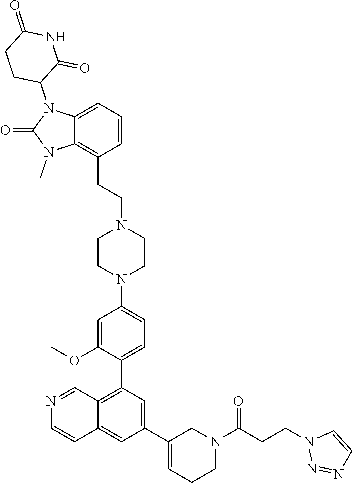 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccncc12] |

In some embodiments, the present invention provides methods of degrading STAT6, or a mutant thereof, or methods of treating a STAT6-mediated disease, disorder, or condition comprising administering a compound set forth in Table 1A, above, or a pharmaceutically acceptable salt thereof. In some embodiments, the present invention provides methods of degrading STAT6, or a mutant thereof, or methods of treating a STAT6-mediated disease, disorder, or condition comprising administering a compound set forth in Table 1A, above.

In some embodiments, the present invention provides a compound described herein (such as a compound of formula I as defined above), or a pharmaceutically acceptable salt thereof, or a pharmaceutical composition comprising a compound described herein (such as a compound of formula I as defined above), or a pharmaceutically acceptable salt thereof, and a pharmaceutically acceptable carrier, adjuvant, or vehicle for use as a medicament.

In some embodiments, the invention also provides a compound described herein (such as a compound of formula I), or pharmaceutical compositions described herein, for use in a method for degrading STAT6 as described herein and/or in a method for treating a STAT6-mediated disorder as described herein. In some embodiments, the invention also provides a compound described herein (such as a compound of formula I), or pharmaceutical compositions described herein, for use in a method for degrading STAT6 as described herein. In some embodiments, the invention also provides a compound described herein (such as a compound of formula I), or pharmaceutical compositions described herein, for use in a method for treating a STAT6-mediated disorder as described herein.

Another aspect of the present disclosure relates to a method of treating a STAT6-mediated disorder, disease, or condition in a subject in need thereof, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of treating a STAT6-mediated disorder, disease, or condition in a subject in need thereof, wherein the method comprises administering to said subject a compound disclosed herein, or a pharmaceutical acceptable salt thereof; and wherein the method results in the subject having a reduced STAT6 level relative to an untreated subject with the same STAT6-mediated disorder, disease, or condition.

In some embodiments, the STAT6 is from a sample. In some embodiments, a sample comprises a biological sample. In some embodiments, the biological sample is taken from the subject. In some embodiments, the sample comprises a lysate. In some embodiments, the sample comprises a peripheral blood mononuclear cell (PBMC).

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to an untreated subject with the same STAT6-mediated disorder, disease, or condition.

In some embodiments, the STAT6 is from a sample. In some embodiments, the sample comprises a biological sample. In some embodiments, the biological sample is taken from the subject. In some embodiments, the sample comprises a lysate. In some embodiments, the sample comprises a peripheral blood mononuclear cell (PBMC). In some embodiments, the STAT6 is a polypeptide disclosed herein. In some embodiments, the STAT6 is a polypeptide of a plurality of polypeptides disclosed herein.

### 3b. Description of Exemplary Compound and Composition Embodiments

In certain embodiments, the present invention provides a compound of formula I:

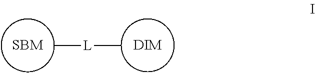

or a pharmaceutically acceptable salt thereof, wherein:
SBM is a STAT6 binding moiety capable of binding to STAT6 protein as described within this section (3b) below and herein;
L is a bivalent moiety that connects SBM to DIM as described within this section (3b) below and herein; and
DIM is a degradation inducing moiety selected from an E3 ubiquitin ligase binding moiety (LBM), lysine mimetic, and hydrogen as described within this section (3b) below and herein.

In some embodiments, the compound of formula I specifically affects, as its primary mechanism of action, the degradation of STAT6.

It will be understood that formula I cannot extend beyond the formulae described within this section (3b) below and herein. It will be understood that all references to “defined and described herein” solely refer to this section (3b) of the application. Additionally, all chemical formulae and embodiments within section 3b can only apply to and can be combined with formulae and embodiments from this section (3b).

### STAT6 Binding Moiety (SBM)

In some embodiments, the present invention provides a compound of formula I-a″″:

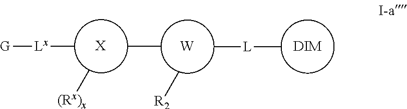

or a pharmaceutically acceptable salt thereof, wherein L and DIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from 3-8 membered saturated or partially unsaturated carbocyclylenyl or heterocyclylenyl with 1-3 heteroatoms independently selected from nitrogen, oxygen, and sulfur, and 5 membered heteroarylenyl with 1-4 heteroatoms independently selected from nitrogen, oxygen and sulfur;
G is hydrogen, halogen, or

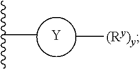

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
wherein R^{y} and R^{x} are not indolyl or azaindolyl;
each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B} is independently -L^{B}-Cy^{L1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and v are independently 0, 1, 2, 3, or 4;
with the proviso that the compound or pharmaceutically acceptable salt thereof is other than a compound of formula I-a″″-1:

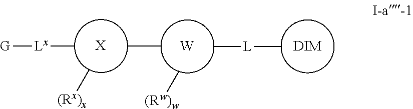

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-00′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, I-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2, I-ccc-3′, I-ccc′-1′, I-ccc″-1′, I-ccc-A, I-ccc-B, I-ccc-C, I-ddd, I-fff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
   G is hydrogen, halogen, or

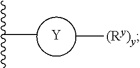

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

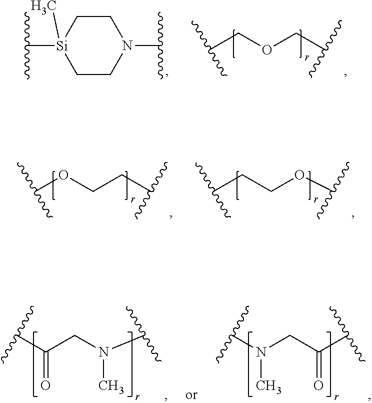

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10;
   or
(ii) @-L¹-L²-L³-L⁴-
   wherein
   @ represents the point of attachment to Ring W;
   L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—:
   L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—: and
   each of -Cy^{L1}-Cy^{L2}-, -Cy^{L3}-, and -Cy^{L4}- is independently -Cy-.

In some embodiments, the present invention provides a compound of formula I-a″″:

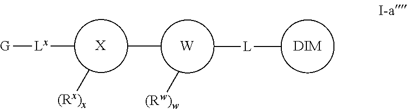

or a pharmaceutically acceptable salt thereof, wherein L and DIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

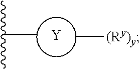

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
wherein R^{y} and R^{x} are not indolyl or azaindolyl;
each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur,
each R^{B} is independently -L^{B}-Cy^{B1}-H or -L^{B}-Cy^{L1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur;
wherein -Cy^{x}- is not indolyl or azaindolyl; and
each of w, x, and y are independently 0, 1, 2, 3, or 4;
with the proviso that the compound or pharmaceutically acceptable salt thereof is other than a compound of formula I-a″″-1:

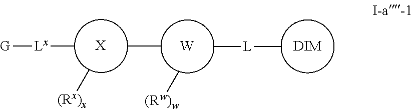

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5′, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, I-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2′, I-ccc-3′, I-ccc′-1′, I-ccc″-1′, I-ccc-A, I-ccc-B, I-ccc-C, I-ddd, I-fff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
   G is hydrogen, halogen, or

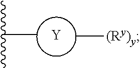

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

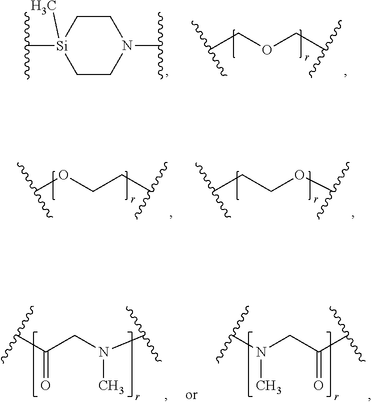

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10:
   or
(ii) @-L¹-L²-L³-L⁴-
   wherein
   @ represents the point of attachment to Ring W;
   L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
   each of -Cy^{L1}-, Cy^{L1}-, -Cy^{L3}, and -Cy^{L4}- is independently -Cy-.

In some embodiments, the present invention provides a compound of formula I-a″″:

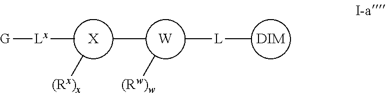

or a pharmaceutically acceptable salt thereof, wherein L and DIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is

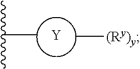

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR—NRC(O)R, —NRC(O)N(R)₂, and —NRS(O)₂R:
wherein R^{y} and R^{x} are not indolyl or azaindolyl;
each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur,
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—:
each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and y are independently 0, 1, 2, 3, or 4;
with the proviso that the compound or pharmaceutically acceptable salt thereof is other than a compound of formula I-a″″-1:

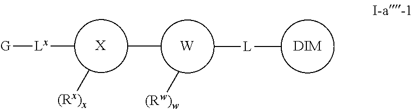

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5′, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, I-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, 1-aaa-12, I-aaa-13, I-aaa-14, 1-aaa-15, 1-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2′, I-ccc-3′, I-ccc′-1′, I-ccc″-1′, I-ccc-A, I-ccc-B, I-ccc-C, I-ddd, I-fff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
   G is hydrogen, halogen, or

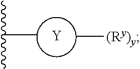

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

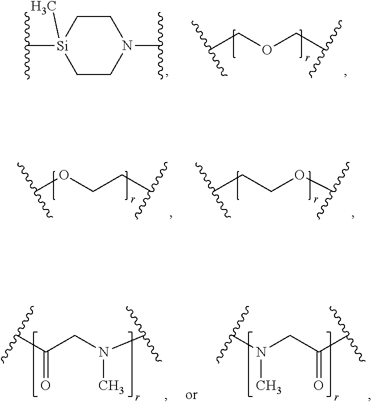

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10;
   or
(ii) @-L¹-L²-L³-L⁴-
   wherein
   @ represents the point of attachment to Ring W;
   L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
   L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—:
   L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
   each of -Cy^{L1}-, Cy^{L2}-, -Cy^{L3}- and -Cy^{L4}- is independently -Cy-.

In some embodiments, the present invention provides a compound of formula I-a″″:

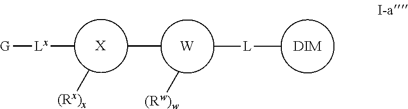

or a pharmaceutically acceptable salt thereof, wherein Ln HIM are as defined and described herein, wherein:
Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-8 or 10-14 membered monocyclic, bicyclic, or tricyclic heteroaryl with 1-4 heteroatoms independently selected from nitrogen, oxygen and sulfur;
Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

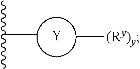

Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-8 or 10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-8 or 10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur,
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-8 or 10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-8 or 10 membered monocyclic or bicyclic aryl or heteroaryl with 1-4 heteroatoms independently selected from nitrogen, oxygen and sulfur;
each R is independently hydrogen, or a group selected from C₁e aliphatic, phenyl, a 4-7 membered saturated or partially unsaturated carbocyclic or heterocyclic ring having 1-2 heteroatoms independently selected from nitrogen, oxygen, and sulfur, and a 5-8 or 10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is a ring selected from phenyl, a 3-7 membered saturated or partially unsaturated carbocyclylenyl or heterocyclylenyl having 1-4 heteroatoms independently selected from nitrogen, oxygen, and sulfur, and a 5-8 membered monocyclic or bicyclic heteroarylenyl having 1-4 heteroatoms independently selected from nitrogen, oxygen, and sulfur; and
each of w, x, and y are independently 0, 1, 2, 3, or 4.

In some embodiments, the present invention provides a compound of formula I-a″″:

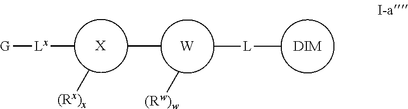

or a pharmaceutically acceptable salt thereof, wherein L and DIM are as defined and described herein, wherein:
Ring W is a ring selected from phenyl, naphthyl, 5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from 3-5 or 7-8 membered saturated or partially unsaturated carbocyclylenyl or heterocyclylenyl with 1-3 heteroatoms independently selected from nitrogen, oxygen, and sulfur, and 5 membered heteroarylenyl with 1-4 heteroatoms independently selected from nitrogen, oxygen and sulfur;
G is hydrogen, halogen, or

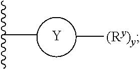

Ring Y is a ring selected from phenyl, naphthyl, 3-5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
wherein R^{y} and R^{x} are not indolyl or azaindolyl:
each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₅ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-5 or 7-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-5 or 7-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or a group selected from C₁₋₅ aliphatic, phenyl, a 4-5 or 7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-5 or 7-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—;
each -Cy^{x}- is a ring selected from phenyl, a 3-5 or 7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and v are independently 0, 1, 2, 3, or 4.

In some embodiments, the present invention provides a compound of formula I-a″″:

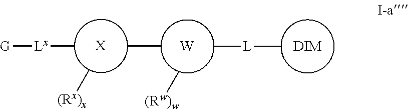

or a pharmaceutically acceptable salt thereof, wherein L and DIM are as defined and described herein, wherein:
Ring W is a ring selected from phenyl, naphthyl, 5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-5 or 7-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is hydrogen, halogen, or

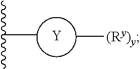

Ring Y is a ring selected from phenyl, naphthyl, 3-5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
wherein R^{y} and R^{x} are not indolyl or azaindolyl;
each R^{w} is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₅ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B} is independently -L^{B}-Cy^{L1}-H or -L^{B}-Cy^{L1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-5 or 7-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-5 or 7-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or a group selected from C₁₋₅ aliphatic, phenyl, a 4-5 or 7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-5 or 7-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—:
each -Cy^{x}- is a ring selected from phenyl, a 3-5 or 7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
wherein -Cy^{x}- is not indolyl or azaindolyl:
each of w, x, and y are independently 0, 1, 2, 3, or 4.

In some embodiments, the present invention provides a compound of formula I-a″″:

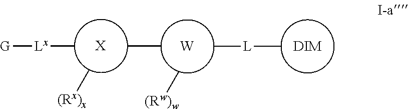

or a pharmaceutically acceptable salt thereof, wherein L and DIM are as defined and described herein, wherein:
Ring W is a ring selected from phenyl, naphthyl, 5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;
Ring X is a ring selected from phenylenyl, 3-5 or 7-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen and sulfur;
G is

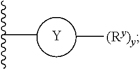

Ring Y is a ring selected from phenyl, naphthyl, 3-5 or 7-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen and sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R:
wherein R^{y} and R^{x} are not indolyl or azaindolyl;
each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently a group selected from C₁₋₅ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.
each R^{B} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently a ring selected from phenylenyl, a 3-5 or 7-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently a ring selected from phenyl, a 3-5 or 7-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or a group selected from C₁₋₅ aliphatic, phenyl, a 4-5 or 7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-5 or 7-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a C₁₋₅ bivalent straight or branched saturated or unsaturated hydrocarbon chain wherein 1-3 methylene units of the chain are independently and optionally replaced with -Cy^{Y}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—:
each -Cy^{x}- is a ring selected from phenyl, a 3-5 or 7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur; and
each of w, x, and v are independently 0, 1, 2, 3, or 4.

It will be understood that, throughout the entirety of this disclosure including all embodiments and formulae within section 3b alone or in combination, the described compounds or pharmaceutically acceptable salts thereof are other than a compound of formula I-a″″-1:

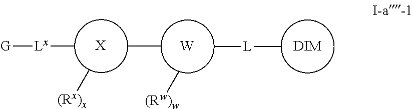

or a pharmaceutically acceptable salt thereof, wherein:
   DIM is any one of formulae I-aa, I-aa′, I-aa-1, I-aa-1′, I-aa-2, I-aa-3, I-aa-4, I-aa-2′, I-aa-3′, I-aa-4′, I-aa-5′, I-aa-6′, I-aa-7′, I-aa-8′, I-aa-9′, I-aa-5a′, I-aa-6a′, I-aa-7a′, I-aa-7b′, I-aa-8a′, I-aa-9a′, I-aa-10′, I-aa-11′, I-aa-12′, I-aa-13′, I-aa-14′, I-aa-10a′, I-aa-11a′, I-aa-12a′, I-aa-12b′, I-aa-13a′, I-aa-14a′, I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, I-oo″-10, I-uu, I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, I-aaa-18, I-aaa-19, I-aaa-20, I-aaa-21, I-bbb-1, I-bbb-2, I-bbb-3, I-bbb-4, I-ccc-1, I-ccc-2, I-ccc-3, I-ccc′-1, I-ccc″-1, I-ccc-1′, I-ccc-2′, I-ccc-3′, I-ccc′-1′, I-ccc-1′, I-ccc-A, I-ccc-B, I-ccc-C, I-ddd, I-ff, I-ggg, I-hhh, I-iii, I-jjj, I-qqq, I-qqq-A, I-qqq-B, I-qqq-1, I-qqq-12, I-rrr, I-sss-1, I-sss-2, I-uuu-1, I-uuu-2, I-uuu-3, I-uuu-4, I-vvv, I-www, I-xxx-1, I-xxx-2, I-yyy-1, I-yyy-2, I-zzz, I-aaaa, I-aaaa-1, I-aaaa-2, I-aaaa-3, I-b, I-b-a, I-b-b, I-b-c, I-b-c-1, I-b-c-2, I-b-c-3, I-b-c-4, I-c-a-1, I-c-a-2, I-bbbb-1, I-cccc-1, I-aaaa, I-aaaa-1, I-aaaa-2, I-bbbb-1, I-bbbb-2, I-bbbb-3, or I-cccc, as described herein;
   Ring W is a 9-membered bicyclic heteroaryl with 1-4 heteroatoms independently selected from nitrogen, oxygen, and sulfur;
   Ring X is a 6-membered saturated or partially unsaturated carbocyclylenyl or heterocyclylenyl with 1-3 heteroatoms independently selected from nitrogen, oxygen, and sulfur;
   L^{X} is a covalent bond or a C₁₋ₛ bivalent straight or branched saturated or unsaturated hydrocarbon chain wherein 1-2 methylene units of the chain are independently and optionally replaced with —O—. —C(O)—, —C(S)—, —CR₂—, —NR—, —S—, —S(O)—, or —S(O)₂—;
   G is hydrogen, halogen, or

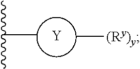

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur; and
L is:
(i) a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF2-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

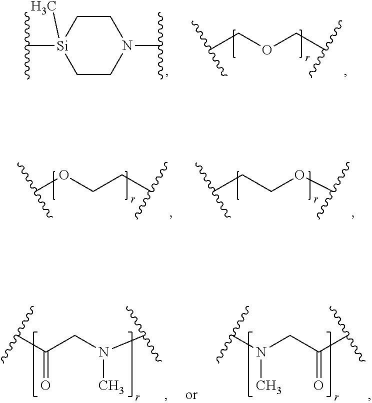

wherein:
   each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
   each r is independently 0, 1, 2, 3, 4, 5, 6, 7, 8, 9, or 10;
   or
(ii) @L¹-L²-L³-L⁴-
   wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—:
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L2}-, Cy^{L2}-, -Cy^{L3}- and -Cy^{L4}- is independently -Cy-.

It will be understood that the embodiments and subgenera below can only be applied to formula I-a″″-1, as it is defined above in the preceding paragraphs. The embodiments and subgenera within this section cannot be combined with formula I-a″″-1, as it is defined above in the preceding paragraphs, in a way that broadens any of the recited definitions of formula I-a″″-1.

As described above and defined herein, Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-14 membered monocyclic, bicyclic, or tricyclic heteroaryl with 1-4 heteroatoms independently selected from nitrogen, oxygen, and sulfur.

In some embodiments, Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-13 membered monocyclic, bicyclic, or tricyclic heteroaryl with 1-4 heteroatoms independently selected from nitrogen, oxygen, and sulfur.

In some embodiments, Ring W is phenyl. In some embodiments, Ring W is naphthyl. In some embodiments, Ring W is a 5-11 membered saturated or partially unsaturated monocyclic, bicyclic, bridged bicyclic, or spirocyclic carbocyclyl. In some embodiments, Ring W is a 5-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring W is a 5-13 membered monocyclic, bicyclic, or tricyclic heteroaryl with 1-4 heteroatoms independently selected from nitrogen, oxygen, and sulfur. In some embodiments, Ring W is a 5-10 membered monocyclic or bicyclic heteroaryl with 1-4 heteroatoms independently selected from nitrogen, oxygen, and sulfur.

In some embodiments, Ring W is a 8-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 8-13 membered bicyclic heteroaryl with 1-4 heteroatoms independently selected from nitrogen, oxygen, and sulfur.

In some embodiments, Ring W is an 8-11 membered saturated or partially unsaturated bicyclic heterocyclyl with 1-3 heteroatoms independently selected from nitrogen, oxygen, and sulfur. In some embodiments, Ring W is a 9-10 membered saturated or partially unsaturated bicyclic heterocyclyl with 1-3 heteroatoms independently selected from nitrogen, oxygen, and sulfur. In some embodiments, Ring W is a 9 membered saturated or partially unsaturated bicyclic heterocyclyl with 1-3 heteroatoms independently selected from nitrogen, oxygen, and sulfur. In some embodiments, Ring W is isisoindolinyl, isoindolinonyl, isoindolinedionyl, pyrrolopyridinyl, pyrrolopyrimidinyl, 6,7-dihydro-5H-pyrrolo[3,4]-d]pyrimidinyl, tetrahydropyrrolo[3,2-c]pyridinyl, tetrahydroindolyl, quinolizinyl, or tetrahydropyrazolo[1,5-a]pyrimidinyl.

In some embodiments, Ring W is a 10 membered saturated or partially unsaturated bicyclic heterocyclyl with 1-3 heteroatoms independently selected from nitrogen, oxygen, and sulfur. In some embodiments, Ring W is chromanyl, chromenyl, isochromenyl, tetrahydroisoquinolinyl, tetrahydroquinolinyl, or 1,2-dihydroquinolinyl, 1,2-dihydroisoquinolinyl.

Ring W is a ring selected from phenyl, naphthyl, 5-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and 5-14 membered monocyclic, bicyclic, or tricyclic heteroaryl with 1-4 heteroatoms independently selected from nitrogen, oxygen and sulfur;

In some embodiments, Ring W is a 5-8 membered saturated or partially unsaturated monocyclic heterocyclyl with 1-4 heteroatoms independently selected from nitrogen, oxygen, and sulfur. In some embodiments, Ring W is a 5-8 membered monocyclic heteroaryl with 1-4 heteroatoms independently selected from nitrogen, oxygen, and sulfur. In some embodiments, Ring W is a 9-10 membered saturated or partially unsaturated bicyclic heterocyclyl with 1-4 heteroatoms independently selected from nitrogen, oxygen, and sulfur. In some embodiments, Ring W is a 9-10 membered heteroaryl with 1-4 heteroatoms independently selected from nitrogen, oxygen, and sulfur. In some embodiments, Ring W is an 11-14-membered tricyclic heteroaryl with 1-4 heteroatoms independently selected from nitrogen, oxygen, and sulfur.

In some embodiments, Ring W is a 5-6 membered heteroaryl with 1-4 heteroatoms independently selected from nitrogen, oxygen, and sulfur. In some embodiments, Ring W is a 5 membered heteroaryl with 1-4 heteroatoms independently selected from nitrogen, oxygen, and sulfur. In some embodiments, Ring W is furanyl, oxazolyl, isoxazolyl, or oxadiazolyl. In some embodiments, Ring W is a 6 membered heteroaryl with 1-4 heteroatoms independently selected from nitrogen, oxygen, and sulfur. In some embodiments, Ring W is pyridinyl, pyrimidinonyl, pyridazinyl, or triazinyl.

In some embodiments, Ring W is a 9-membered bicyclic heteroaryl with 1-4 heteroatoms independently selected from nitrogen, oxygen, and sulfur. In some embodiments, Ring W is a 9-membered bicyclic heteroaryl with 1-2 heteroatoms independently selected from nitrogen, oxygen, and sulfur.

In some embodiments, Ring W is a 9-membered bicyclic heteroaryl with 1 heteroatom independently selected from nitrogen, oxygen, and sulfur. In some embodiments, Ring W is a 9-membered bicyclic heteroaryl with 2 heteroatoms independently selected from nitrogen, oxygen, and sulfur. In some embodiments, Ring W is indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl.

In some embodiments, Ring W is azaindazolyl (e.g., 4-, 5-, 6-, or 7-azaindazolyl). In some embodiments, Ring W is pyrrolol[2,3-c]pyridinyl. In some embodiments, Ring W is indolizinyl.

In some embodiments, Ring W is a 10-membered bicyclic heteroaryl with 1-4 heteroatoms independently selected from nitrogen, oxygen, and sulfur. In some embodiments, Ring W is a 10-membered bicyclic heteroaryl with 1-2 heteroatoms independently selected from nitrogen, oxygen, and sulfur. In some embodiments, Ring W is a 10-membered bicyclic heteroaryl with 1-2 nitrogen heteroatoms. In some embodiments, Ring W is quinolinyl, isoquinolinyl, quinolizinyl, quinoxalinyl, phthalazinyl, quinazolinyl, cinnolinyl, or 1,8-naphthyridinyl.

In some embodiments, Ring W is a 10-14 membered tricyclic heteroaryl with 1-4 heteroatoms independently selected form nitrogen, oxygen, and sulfur. In some embodiments, Ring W is a 13 membered tricyclic heteroaryl with 1-4 heteroatoms independently selected form nitrogen, oxygen, and sulfur. In some embodiments, Ring W is:

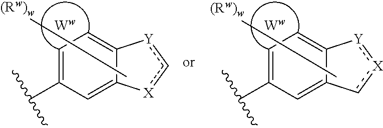

wherein X, Y, R^{w}, and w are as defined above and described herein; and
Ring W^{W} is a fused phenyl or 5-6 membered saturated or partially unsaturated carbocyclyl or heterocyclyl having 1-2 heteroatoms independently selected from nitrogen, oxygen, and sulfur, or a 5-6 membered heteroaryl having 1-2 heteroatoms independently selected from nitrogen, oxygen, and sulfur.

As defined above and described herein, Ring WW is a fused phenyl or 5-6 membered saturated or partially unsaturated carbocyclyl or heterocyclyl having 1-2 heteroatoms independently selected from nitrogen, oxygen, and sulfur, or a 5-6 membered heteroaryl having 1-2 heteroatoms independently selected from nitrogen, oxygen, and sulfur. In some embodiments, Ring WW is a fused phenyl. In some embodiments, Ring Ww is a 5-6 membered saturated or partially unsaturated carbocyclyl. In some embodiments, Ring Ww is a 5-6 membered saturated or partially unsaturated heterocyclyl having 1-2 heteroatoms independently selected from nitrogen, oxygen, and sulfur. In some embodiments, Ring W^{W} is a 5-6 membered heteroaryl having 1-2 heteroatoms independently selected from nitrogen, oxygen, and sulfur. In some embodiments, Ring W is

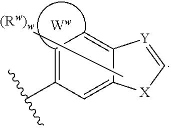

It will be understood that, in the preceding or following paragraphs where only a single point of attachment

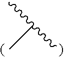

at Ring W is depicted, the depicted

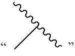

represents the point of attachment to Ring X, and an additional point of attachment to -L-DIM may replace an —H at any point on Ring W as allowed by valency.

In some embodiments, Ring W is

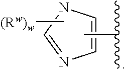

In some embodiments, Ring W is

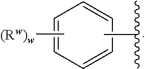

In some embodiments, Ring W is

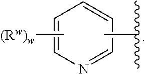

In some embodiments, Ring W is

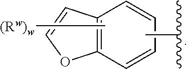

In some embodiments, Ring W is

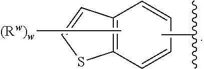

In some embodiments, Ring W is

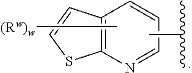

In some embodiments, Ring W is

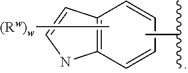

In some embodiments, Ring W is

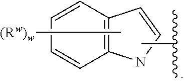

In some embodiments, Ring W is

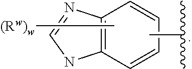

In some embodiments, Ring W is

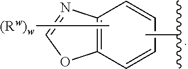

In some embodiments, Ring W is

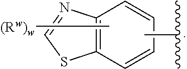

In some embodiments, Ring W is

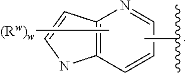

In some embodiments, Ring W is

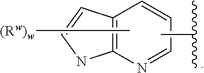

In some embodiments, Ring W is

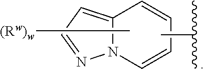

In some embodiments, Ring W is

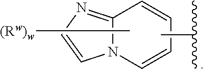

In some embodiments, Ring W is

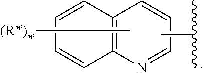

In some embodiments, Ring W is

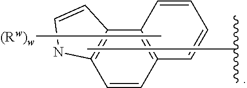

In some embodiments, Ring W is

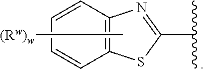

In some embodiments, Ring W is

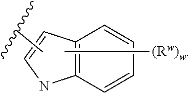

In some embodiments, Ring W is

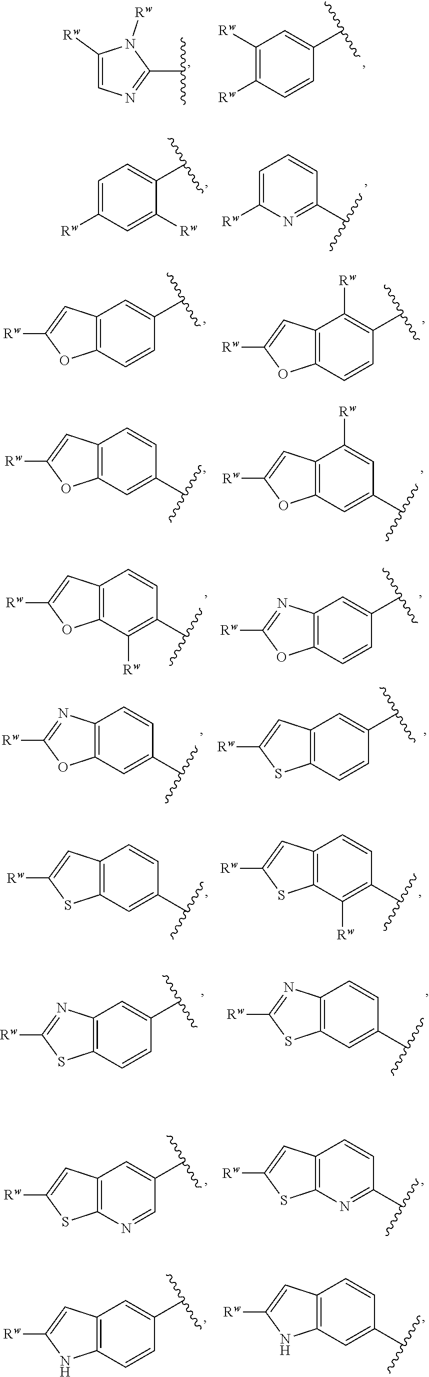

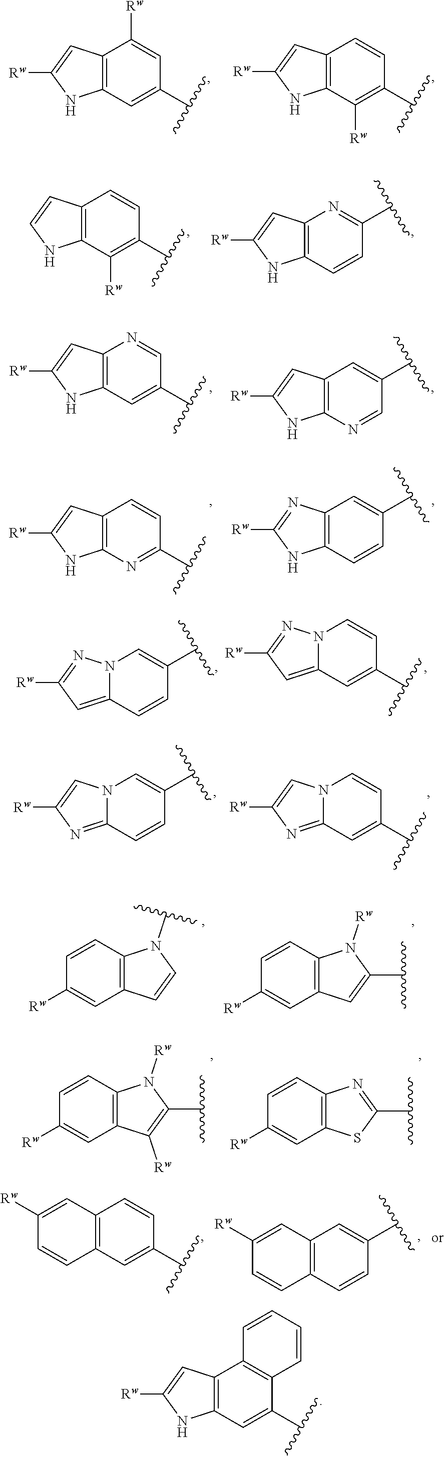

In some embodiment, Ring W is as depicted in the compounds of Table 1B below.

In some embodiments, Ring W is

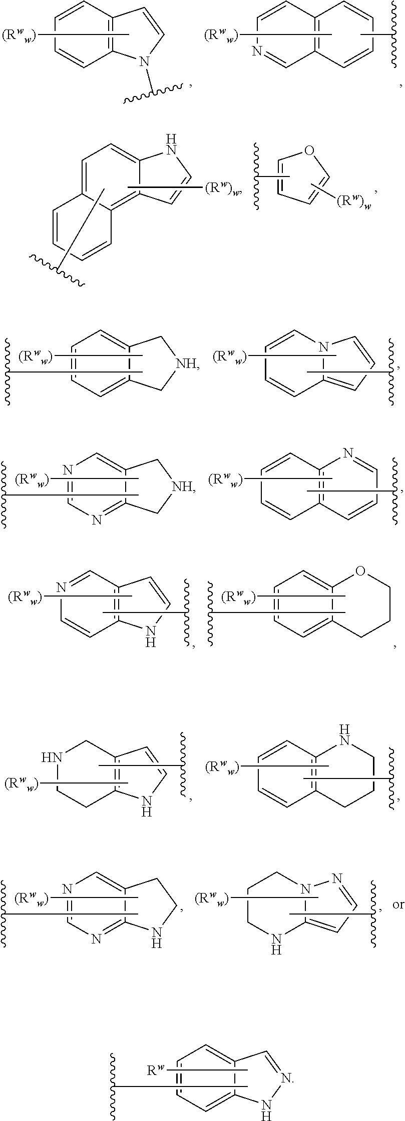

In some embodiments, Ring W is

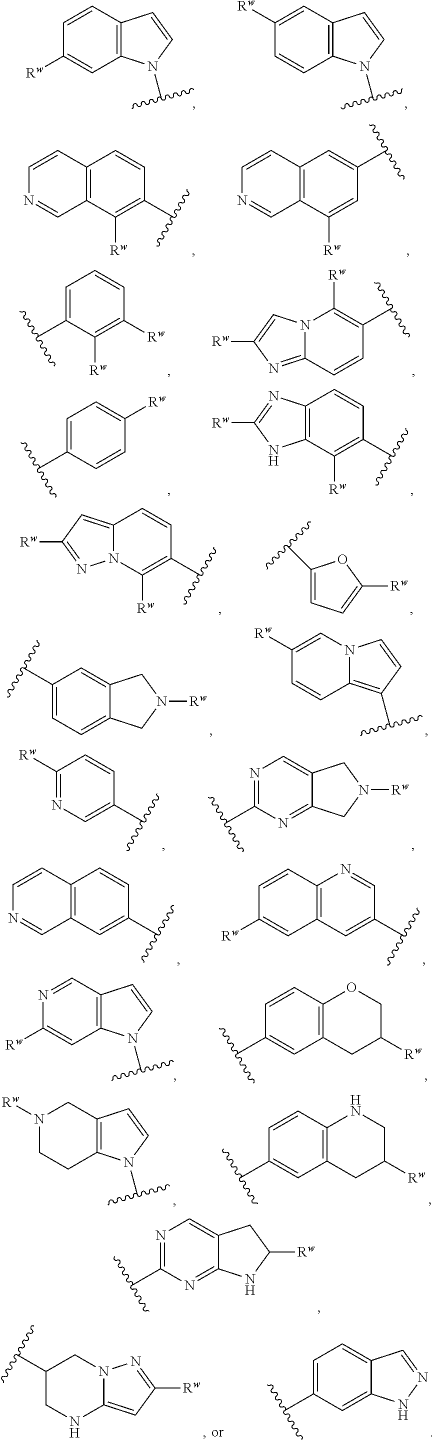

In some embodiments, Ring W is

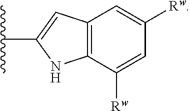

In some embodiments, Ring W is

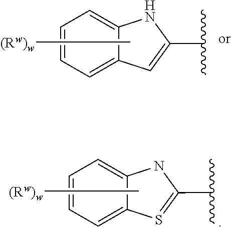

In some embodiments, Ring W is

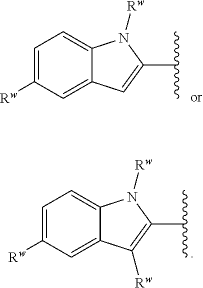

In some embodiments, Ring W is

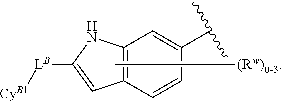

In some embodiments, Ring W is

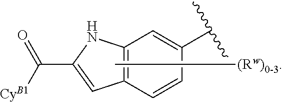

In some embodiments, Ring W and its R^{w} substituents are

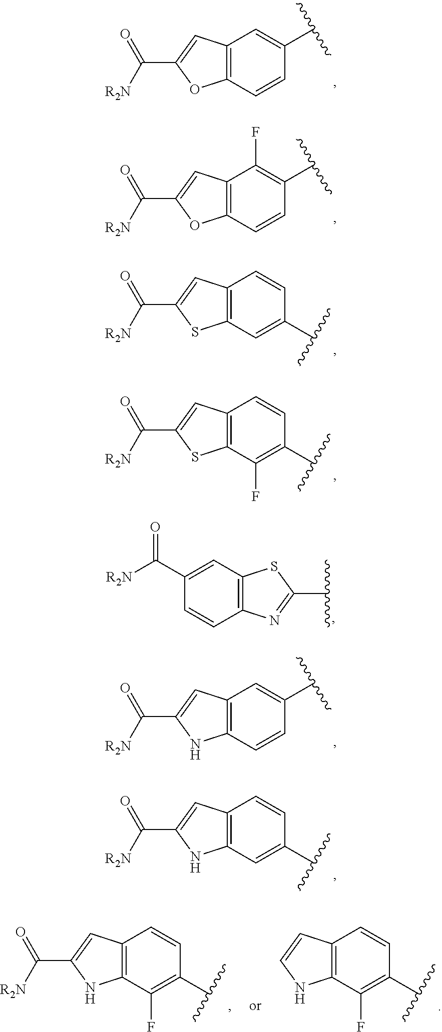

In some embodiments, Ring W and its R^{w} substituents are

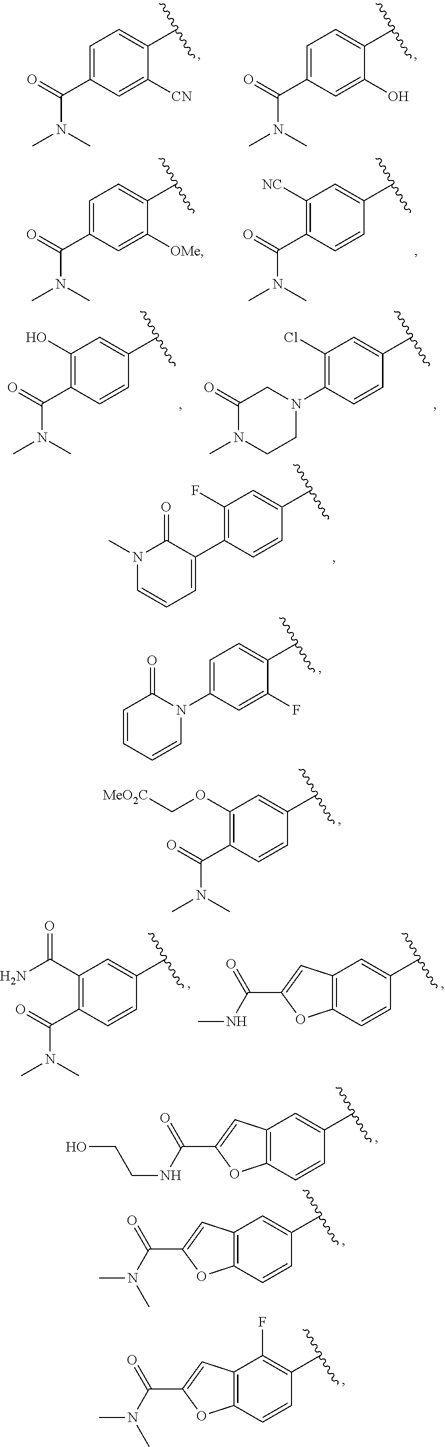

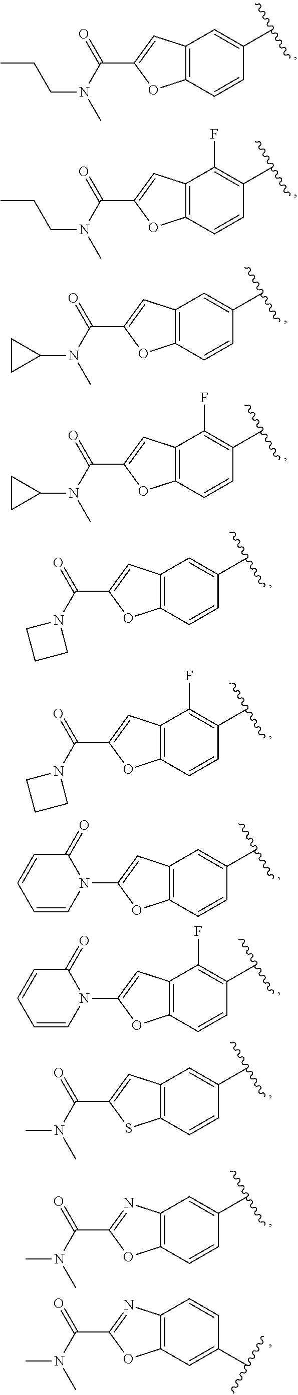

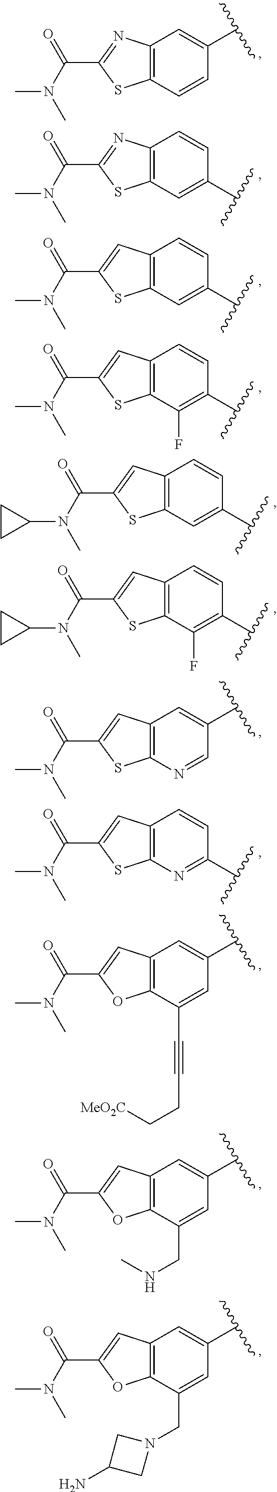

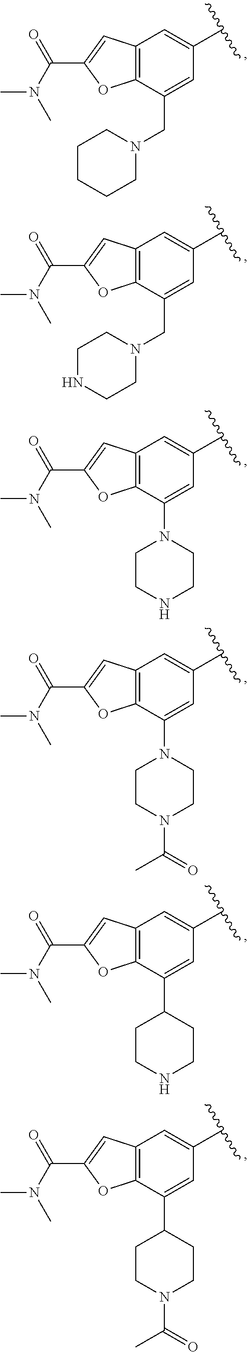

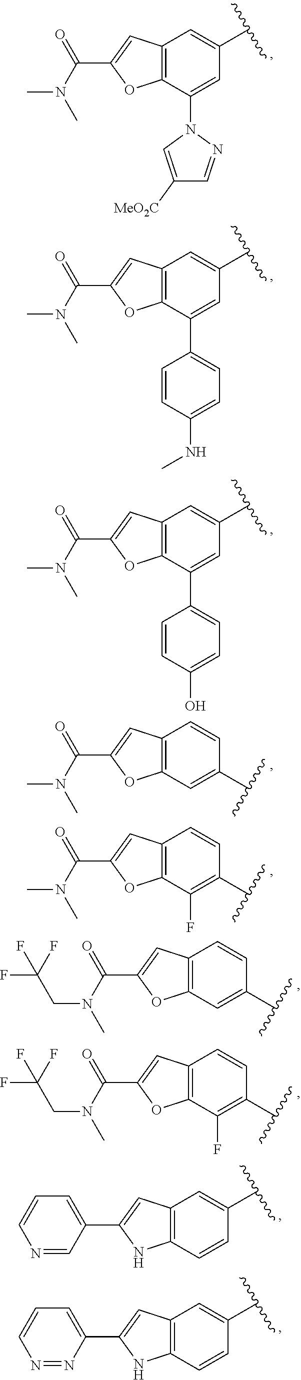

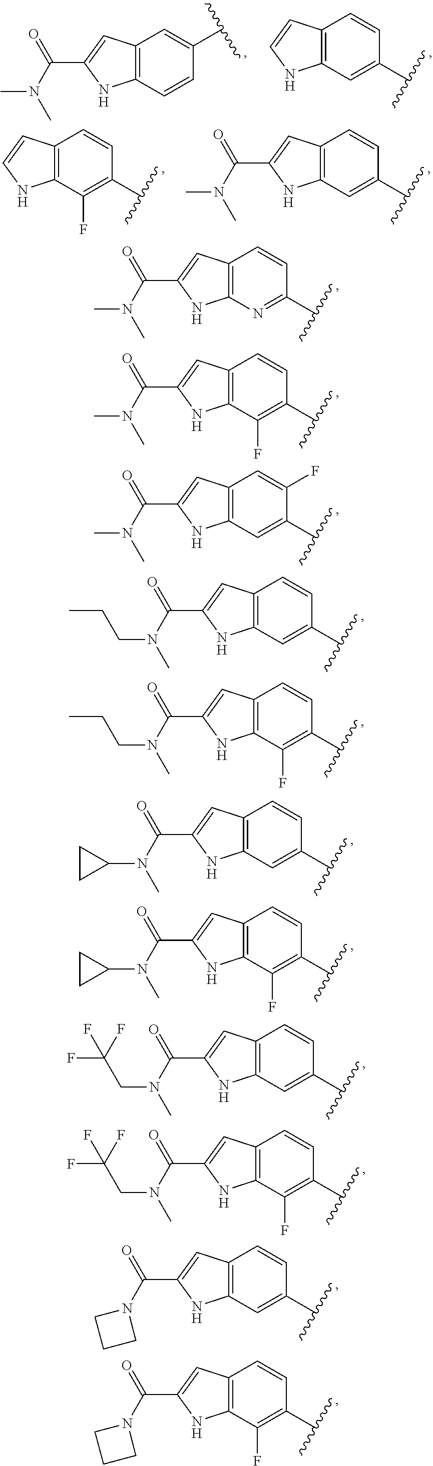

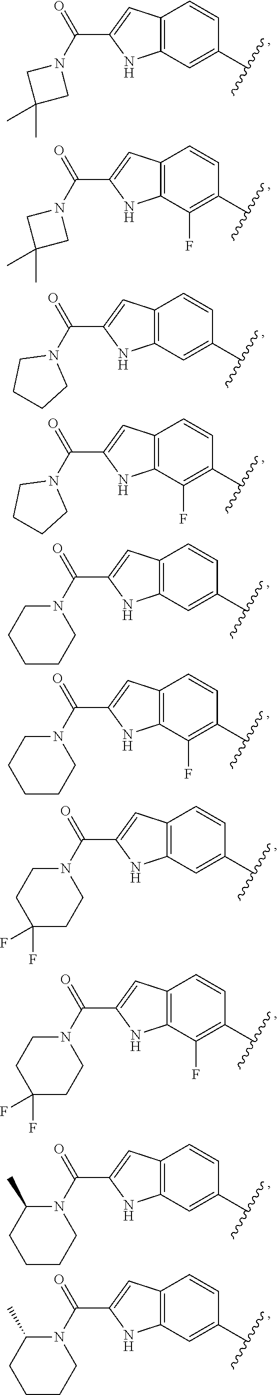

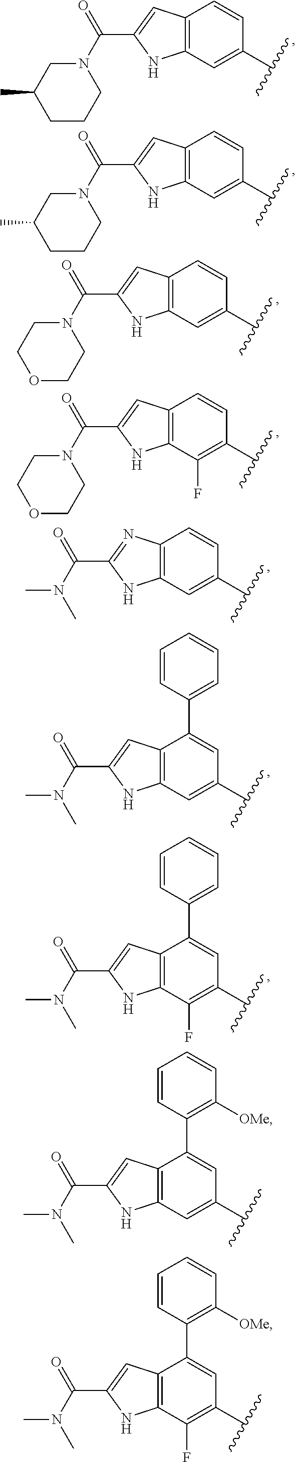

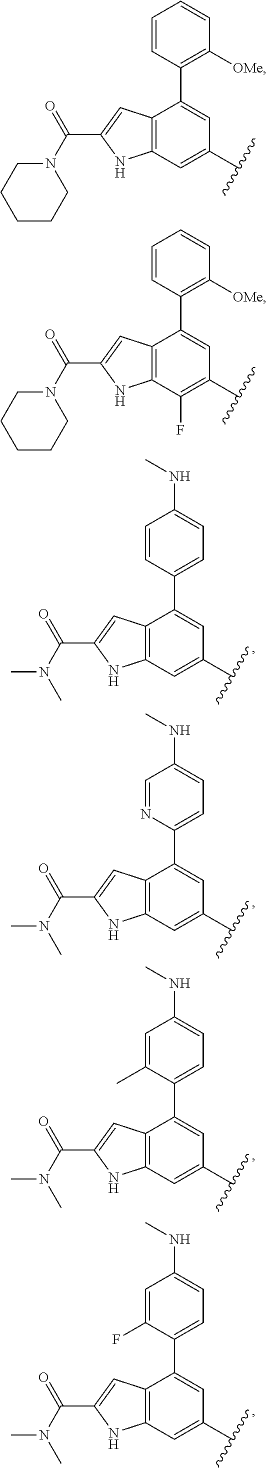

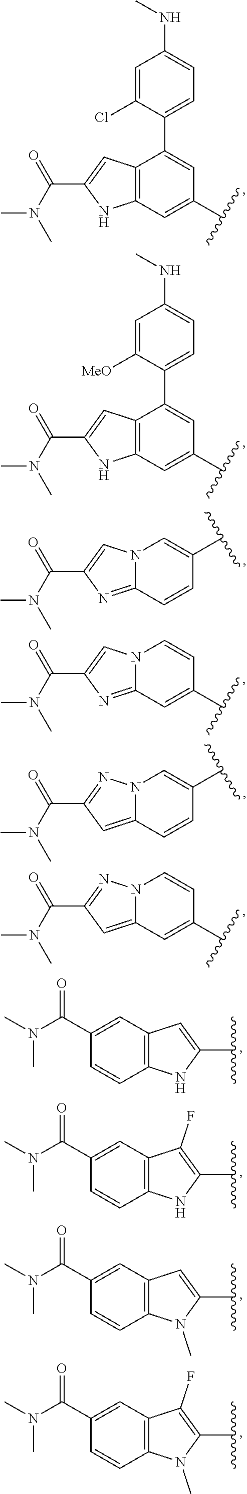

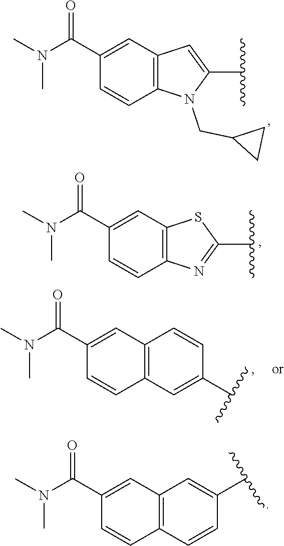

In some embodiments, Ring Wand its Wv substituents are

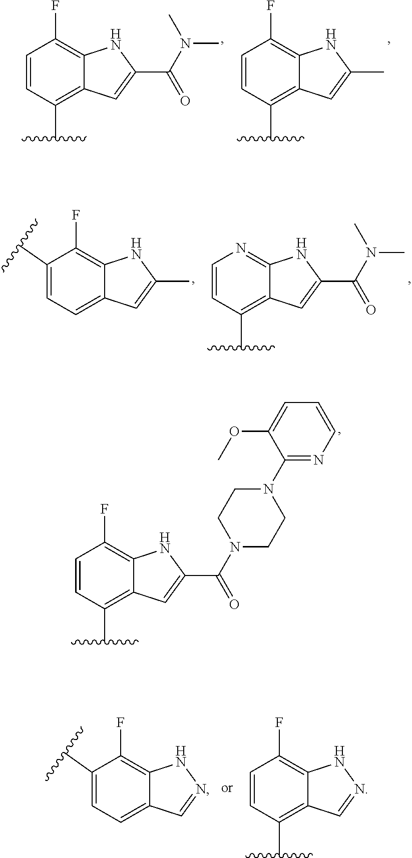

In some embodiments, Ring W and its Wv substituents are

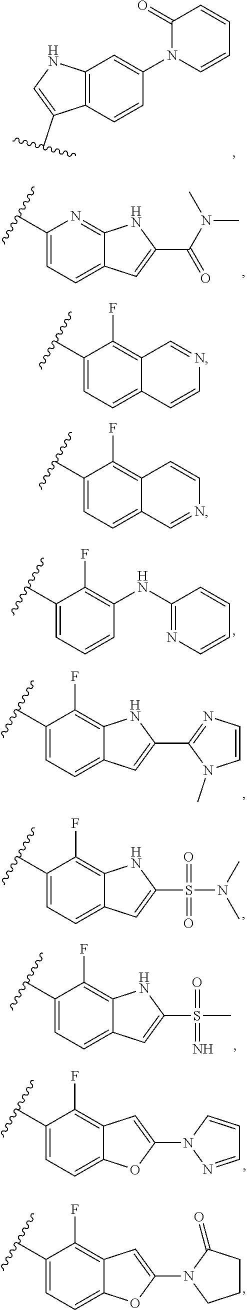

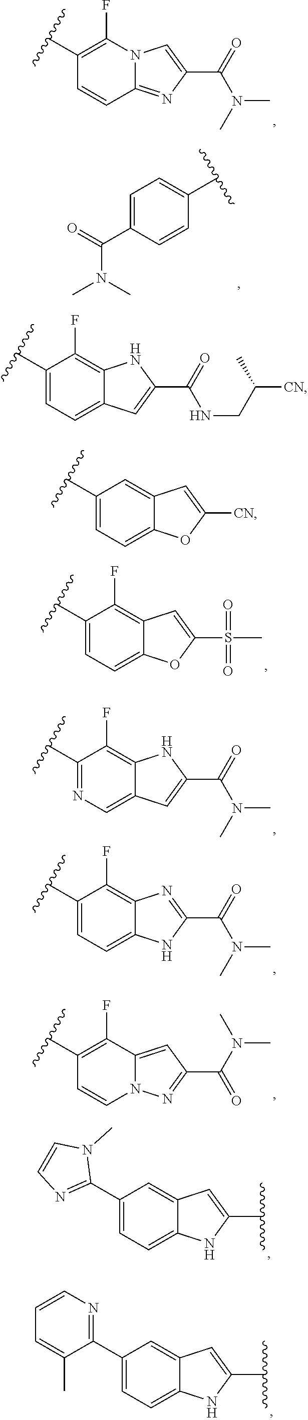

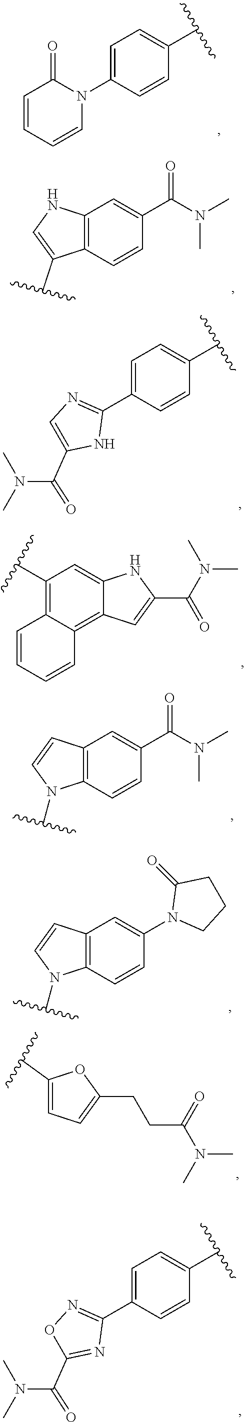

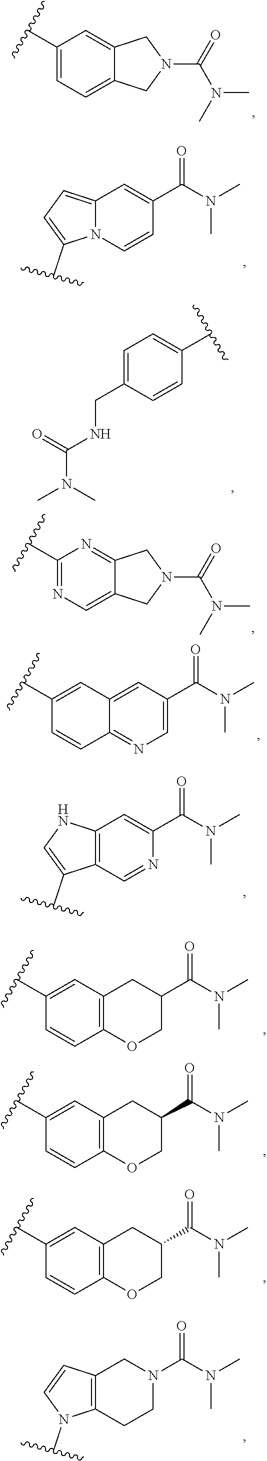

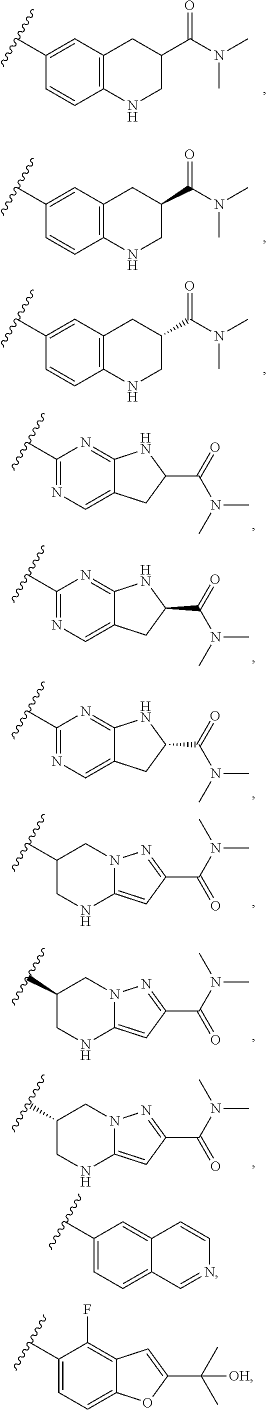

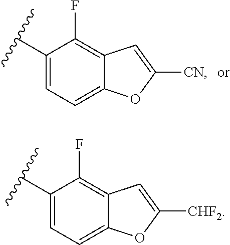

In some embodiments, Ring W and its R^{W} substituents are

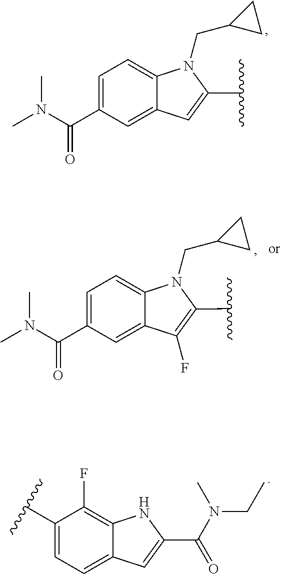

In some embodiments, Ring W and its R^{W} substituents are

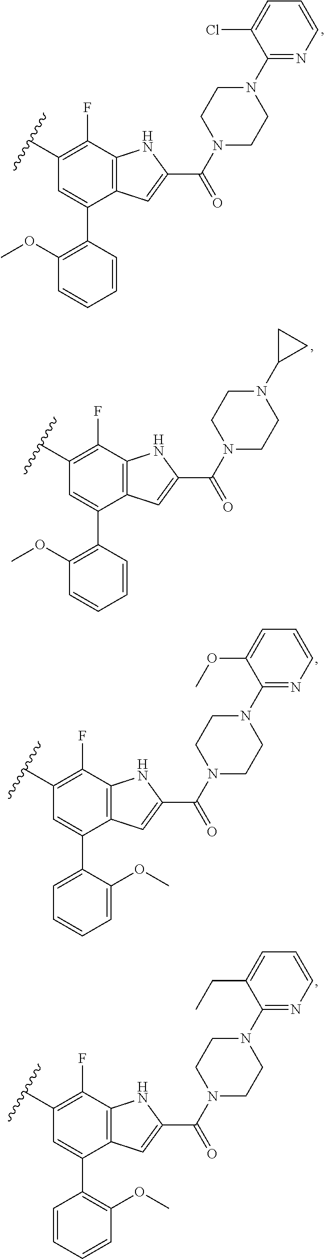 [SMILES: *c1cc(-c2ccccc2OC)c2cc(C(=O)N3CCN(c4ncccc4Cl)CC3)[nH]c2c1F ; *c1cc(-c2ccccc2OC)c2cc(C(=O)N3CCN(C4CC4)CC3)[nH]c2c1F ; *c1cc(-c2ccccc2OC)c2cc(C(=O)N3CCN(c4ncccc4OC)CC3)[nH]c2c1F ; *c1cc(-c2ccccc2OC)c2cc(C(=O)N3CCN(c4ncccc4CC)CC3)[nH]c2c1F]

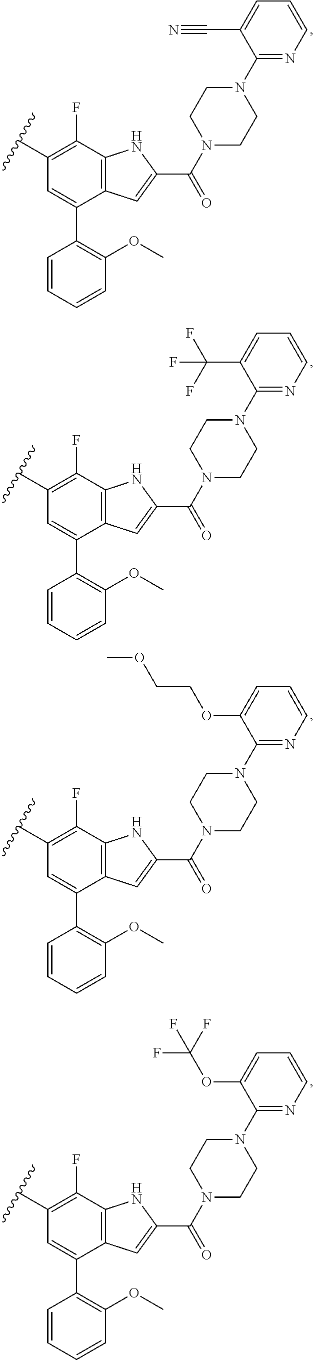

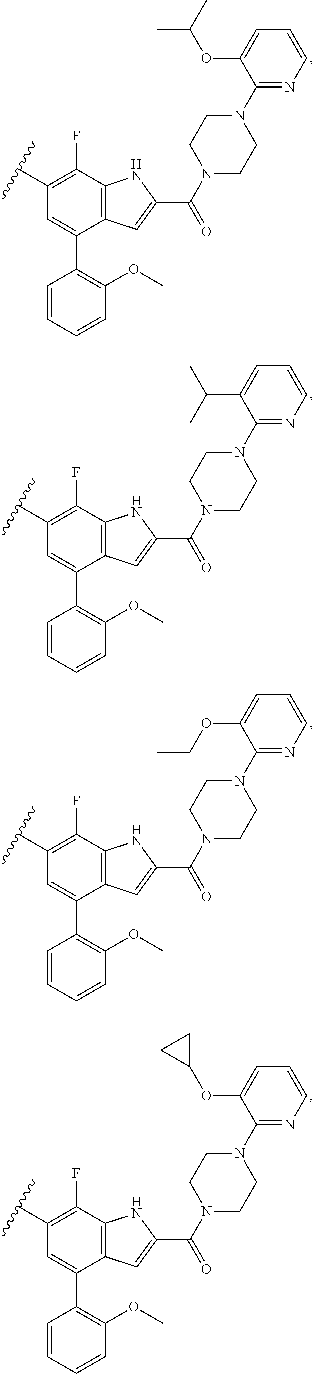 [SMILES: *c1cc(-c2ccccc2OC)c2cc(C(=O)N3CCN(c4ncccc4OC(C)C)CC3)[nH]c2c1F ; *c1cc(-c2ccccc2OC)c2cc(C(=O)N3CCN(c4ncccc4C(C)C)CC3)[nH]c2c1F ; *c1cc(-c2ccccc2OC)c2cc(C(=O)N3CCN(c4ncccc4OCC)CC3)[nH]c2c1F ; *c1cc(-c2ccccc2OC)c2cc(C(=O)N3CCN(c4ncccc4OC4CC4)CC3)[nH]c2c1F]

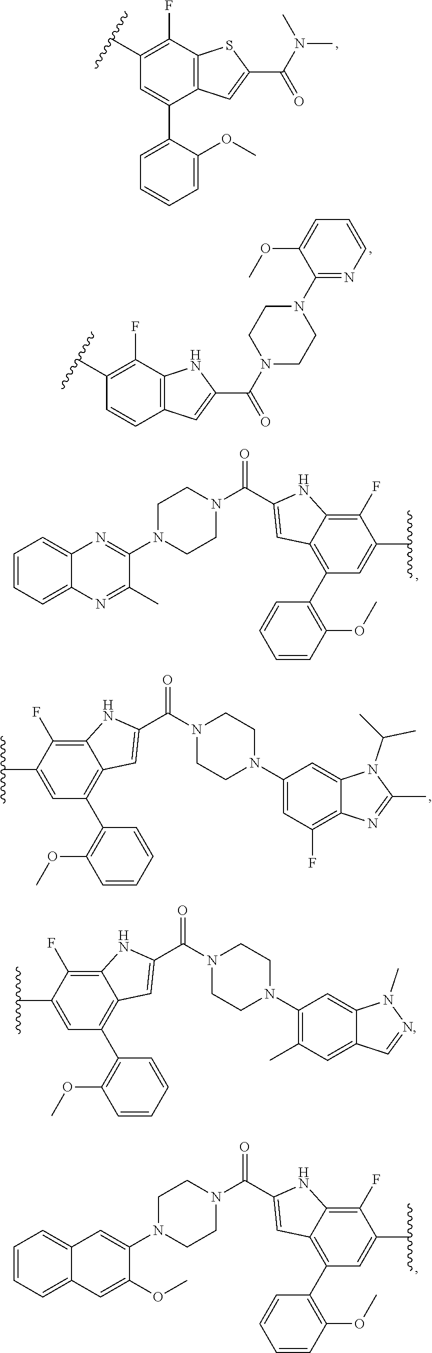

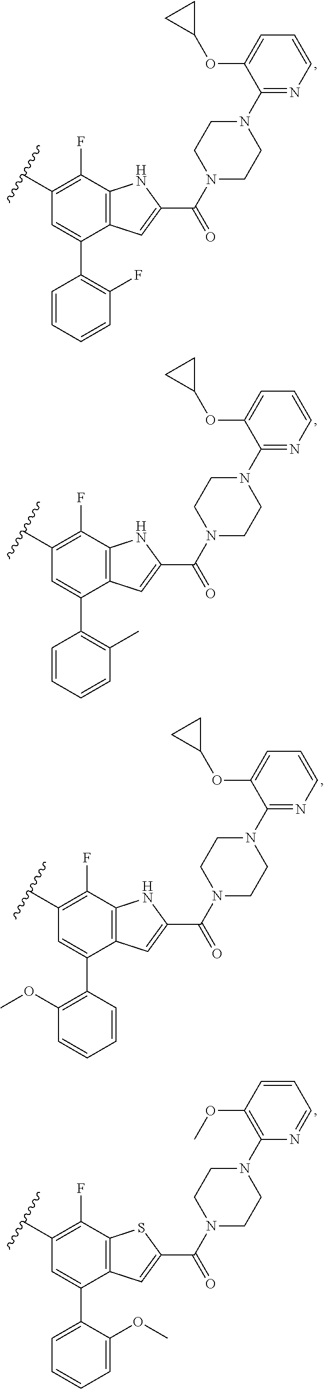 [SMILES: *c1cc(-c2ccccc2F)c2cc(C(=O)N3CCN(c4ncccc4OC4CC4)CC3)[nH]c2c1F ; *c1cc(-c2ccccc2C)c2cc(C(=O)N3CCN(c4ncccc4OC4CC4)CC3)[nH]c2c1F ; *c1cc(-c2ccccc2OC)c2cc(C(=O)N3CCN(c4ncccc4OC4CC4)CC3)[nH]c2c1F ; *c1cc(-c2ccccc2OC)c2cc(C(=O)N3CCN(c4ncccc4OC)CC3)sc2c1F]

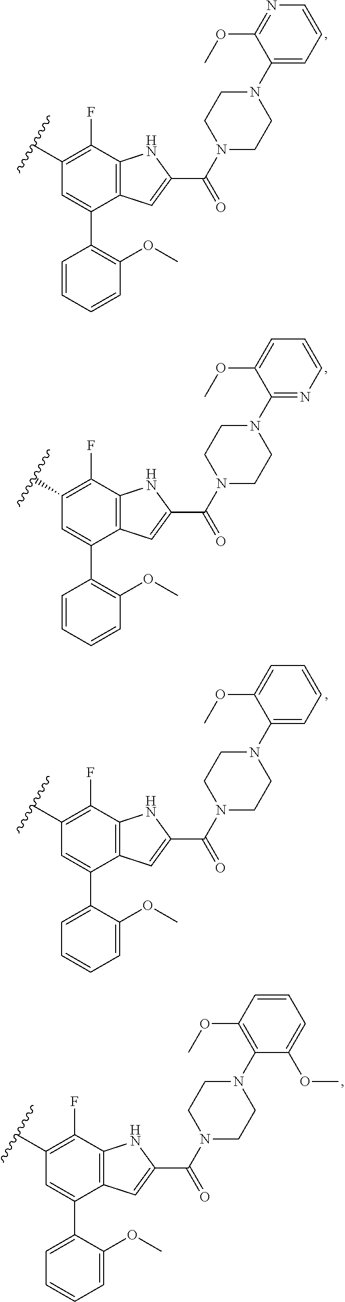

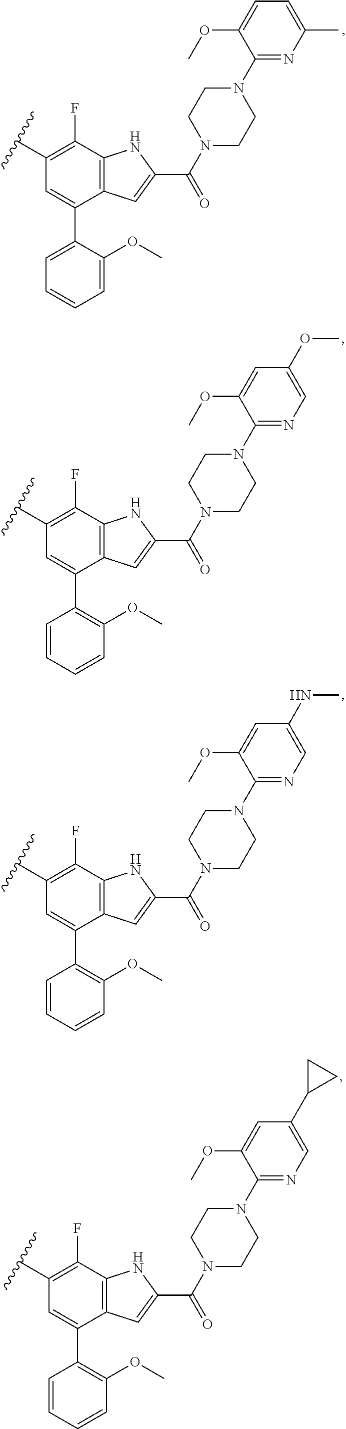 [SMILES: *c1cc(-c2ccccc2OC)c2cc(C(=O)N3CCN(c4nc(C)ccc4OC)CC3)[nH]c2c1F ; *c1cc(-c2ccccc2OC)c2cc(C(=O)N3CCN(c4ncc(OC)cc4OC)CC3)[nH]c2c1F ; *c1cc(-c2ccccc2OC)c2cc(C(=O)N3CCN(c4ncc(NC)cc4OC)CC3)[nH]c2c1F ; *c1cc(-c2ccccc2OC)c2cc(C(=O)N3CCN(c4ncc(C5CC5)cc4OC)CC3)[nH]c2c1F]

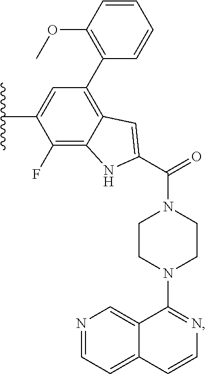 [SMILES: *c1cc(-c2ccccc2OC)c2cc(C(=O)N3CCN(c4nccc5ccncc45)CC3)[nH]c2c1F]

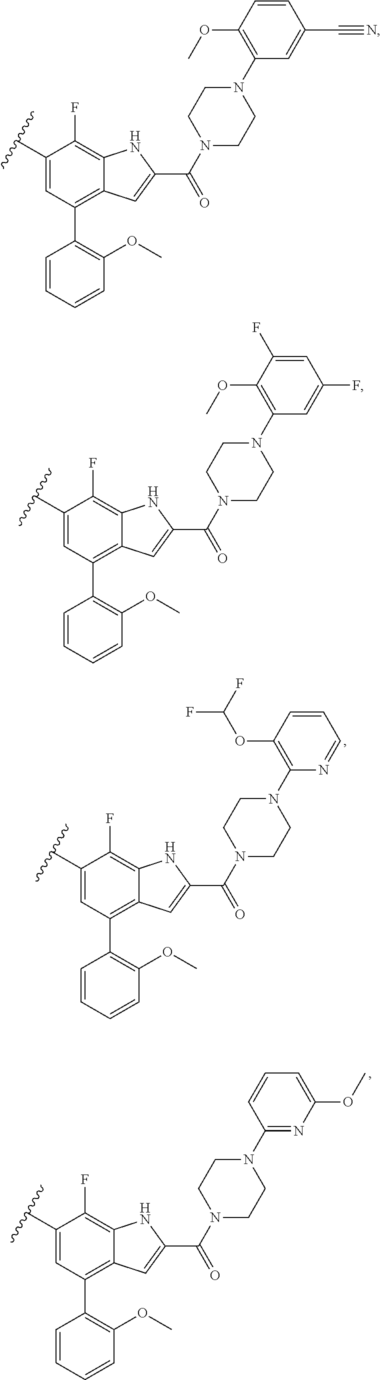 [SMILES: *c1cc(-c2ccccc2OC)c2cc(C(=O)N3CCN(c4cc(C#N)ccc4OC)CC3)[nH]c2c1F ; *c1cc(-c2ccccc2OC)c2cc(C(=O)N3CCN(c4cc(F)cc(F)c4OC)CC3)[nH]c2c1F ; *c1cc(-c2ccccc2OC)c2cc(C(=O)N3CCN(c4ncccc4OC(F)F)CC3)[nH]c2c1F ; *c1cc(-c2ccccc2OC)c2cc(C(=O)N3CCN(c4cccc(OC)n4)CC3)[nH]c2c1F]

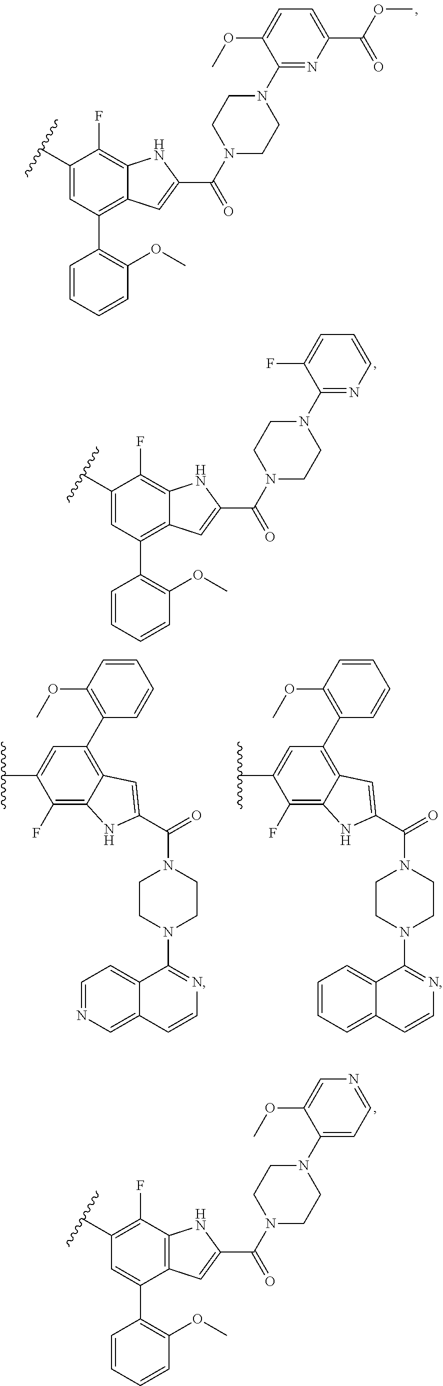

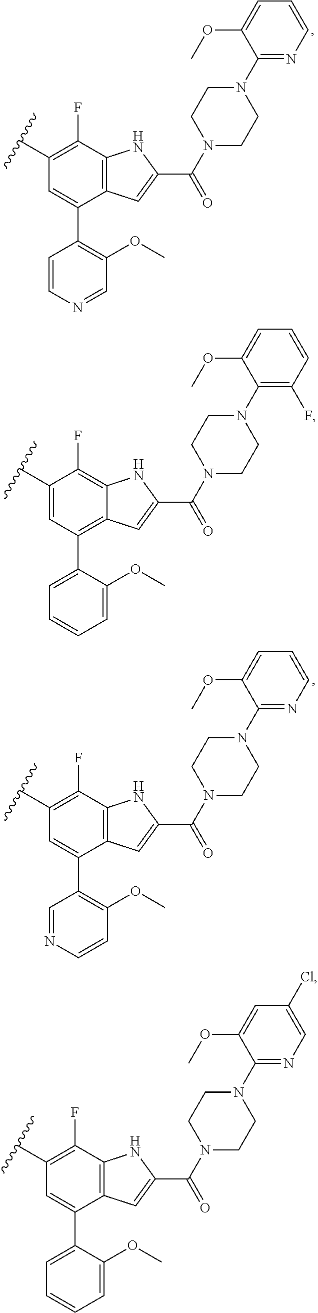 [SMILES: *c1cc(-c2ccncc2OC)c2cc(C(=O)N3CCN(c4ncccc4OC)CC3)[nH]c2c1F ; *c1cc(-c2ccccc2OC)c2cc(C(=O)N3CCN(c4c(F)cccc4OC)CC3)[nH]c2c1F ; *c1cc(-c2cnccc2OC)c2cc(C(=O)N3CCN(c4ncccc4OC)CC3)[nH]c2c1F ; *c1cc(-c2ccccc2OC)c2cc(C(=O)N3CCN(c4ncc(Cl)cc4OC)CC3)[nH]c2c1F]

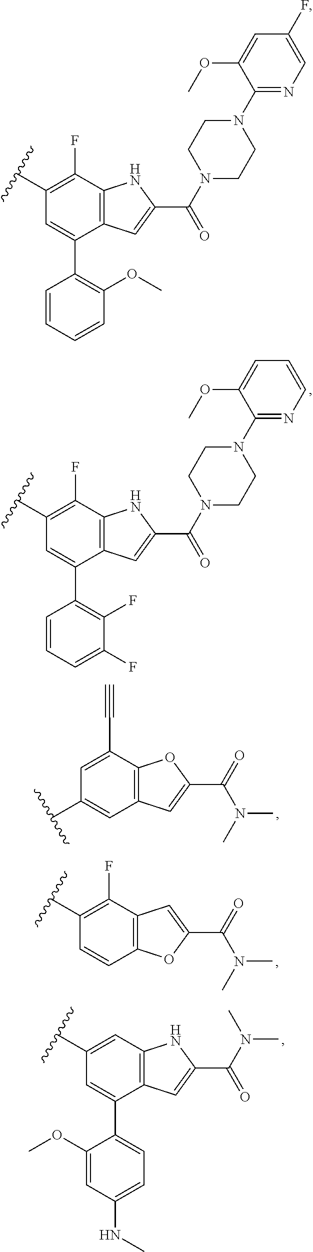

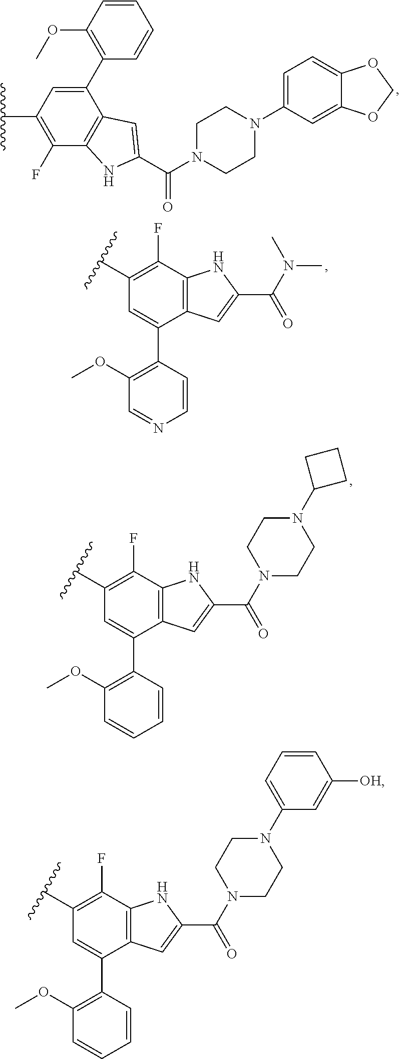

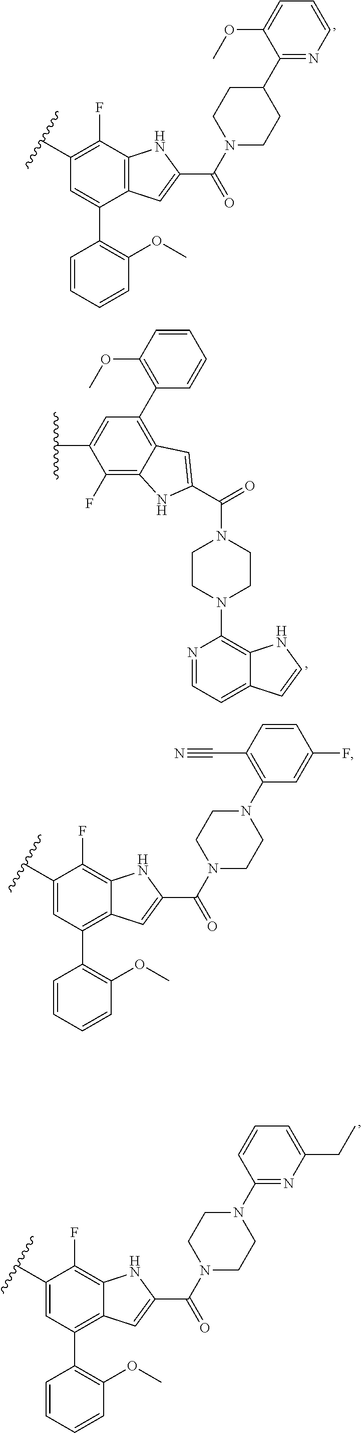

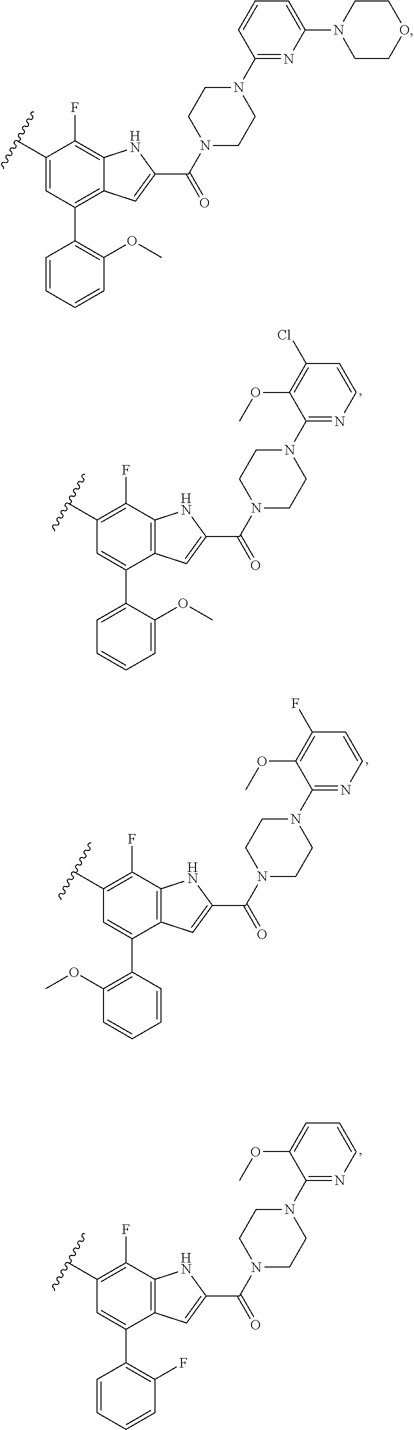 [SMILES: *c1cc(-c2ccccc2OC)c2cc(C(=O)N3CCN(c4cccc(N5CCOCC5)n4)CC3)[nH]c2c1F ; *c1cc(-c2ccccc2OC)c2cc(C(=O)N3CCN(c4nccc(Cl)c4OC)CC3)[nH]c2c1F ; *c1cc(-c2ccccc2OC)c2cc(C(=O)N3CCN(c4nccc(F)c4OC)CC3)[nH]c2c1F ; *c1cc(-c2ccccc2F)c2cc(C(=O)N3CCN(c4ncccc4OC)CC3)[nH]c2c1F]

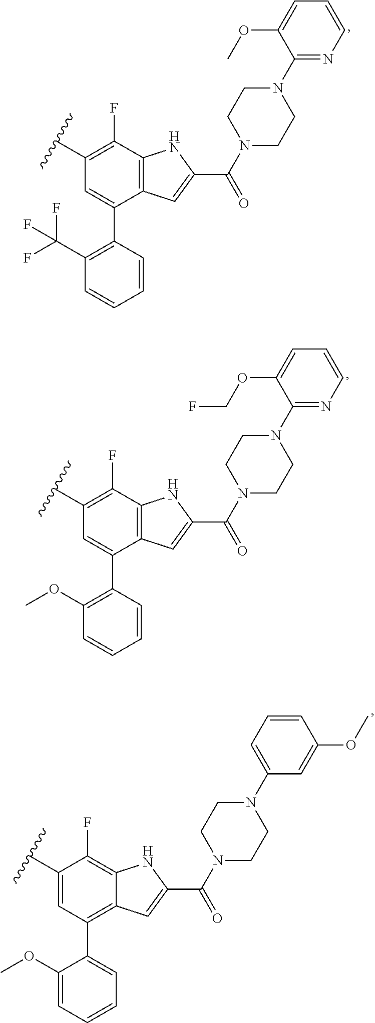

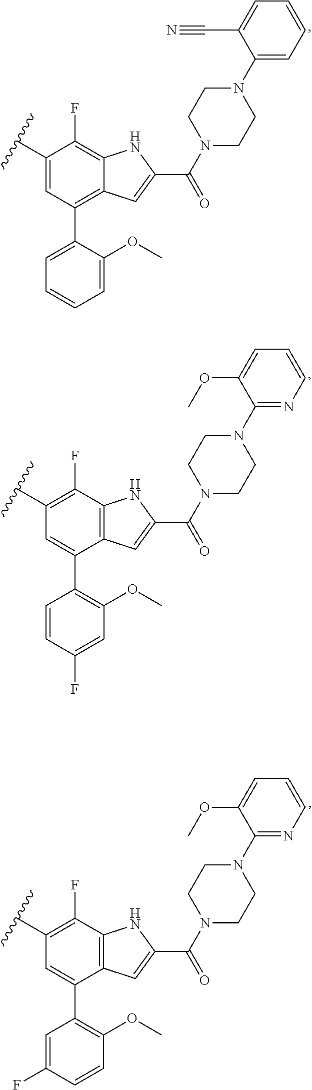 [SMILES: *c1cc(-c2ccccc2OC)c2cc(C(=O)N3CCN(c4ccccc4C#N)CC3)[nH]c2c1F ; *c1cc(-c2ccc(F)cc2OC)c2cc(C(=O)N3CCN(c4ncccc4OC)CC3)[nH]c2c1F ; *c1cc(-c2cc(F)ccc2OC)c2cc(C(=O)N3CCN(c4ncccc4OC)CC3)[nH]c2c1F]

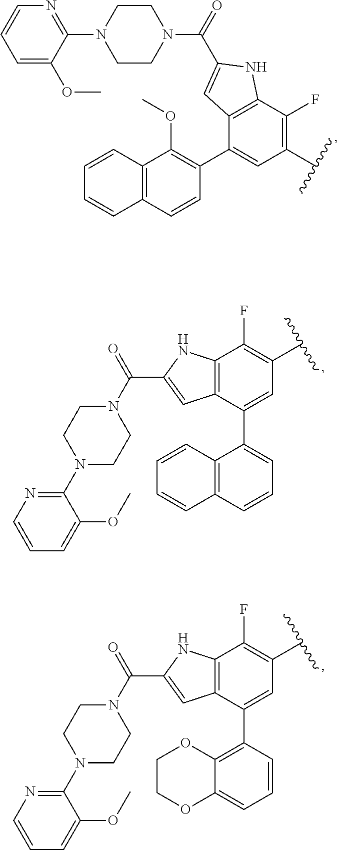 [SMILES: *c1cc(-c2ccc3ccccc3c2OC)c2cc(C(=O)N3CCN(c4ncccc4OC)CC3)[nH]c2c1F ; *c1cc(-c2cccc3ccccc23)c2cc(C(=O)N3CCN(c4ncccc4OC)CC3)[nH]c2c1F ; *c1cc(-c2cccc3c2OCCO3)c2cc(C(=O)N3CCN(c4ncccc4OC)CC3)[nH]c2c1F]

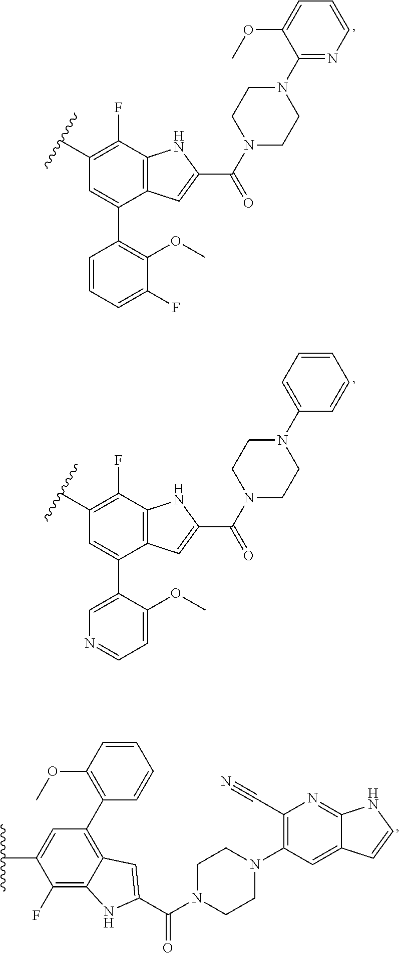 [SMILES: *c1cc(-c2cccc(F)c2OC)c2cc(C(=O)N3CCN(c4ncccc4OC)CC3)[nH]c2c1F ; *c1cc(-c2cnccc2OC)c2cc(C(=O)N3CCN(c4ccccc4)CC3)[nH]c2c1F ; *c1cc(-c2ccccc2OC)c2cc(C(=O)N3CCN(c4cc5cc[nH]c5nc4C#N)CC3)[nH]c2c1F]

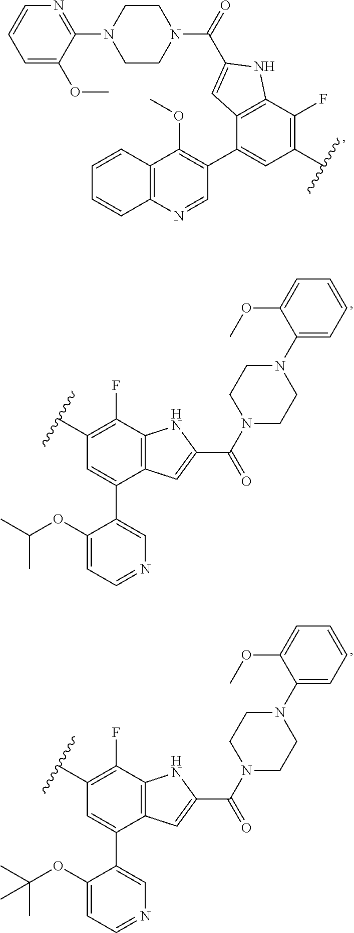 [SMILES: *c1cc(-c2cnc3ccccc3c2OC)c2cc(C(=O)N3CCN(c4ncccc4OC)CC3)[nH]c2c1F ; *c1cc(-c2cnccc2OC(C)C)c2cc(C(=O)N3CCN(c4ccccc4OC)CC3)[nH]c2c1F ; *c1cc(-c2cnccc2OC(C)(C)C)c2cc(C(=O)N3CCN(c4ccccc4OC)CC3)[nH]c2c1F]

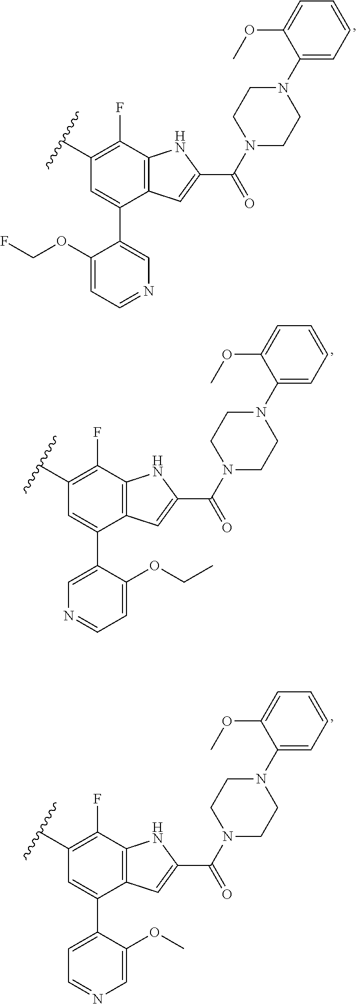 [SMILES: *c1cc(-c2cnccc2OCF)c2cc(C(=O)N3CCN(c4ccccc4OC)CC3)[nH]c2c1F ; *c1cc(-c2cnccc2OCC)c2cc(C(=O)N3CCN(c4ccccc4OC)CC3)[nH]c2c1F ; *c1cc(-c2ccncc2OC)c2cc(C(=O)N3CCN(c4ccccc4OC)CC3)[nH]c2c1F]

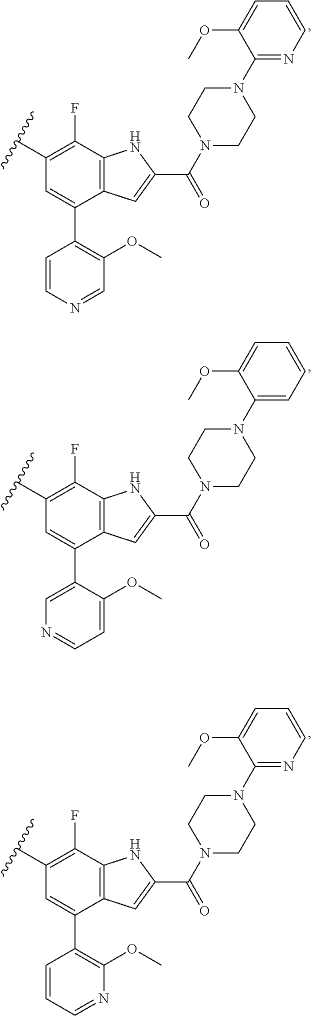 [SMILES: *c1cc(-c2ccncc2OC)c2cc(C(=O)N3CCN(c4ncccc4OC)CC3)[nH]c2c1F ; *c1cc(-c2cnccc2OC)c2cc(C(=O)N3CCN(c4ccccc4OC)CC3)[nH]c2c1F ; *c1cc(-c2cccnc2OC)c2cc(C(=O)N3CCN(c4ncccc4OC)CC3)[nH]c2c1F]

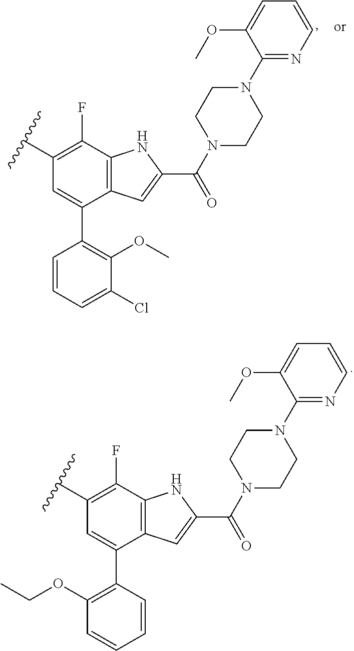 [SMILES: *c1cc(-c2cccc(Cl)c2OC)c2cc(C(=O)N3CCN(c4ncccc4OC)CC3)[nH]c2c1F ; *c1cc(-c2ccccc2OCC)c2cc(C(=O)N3CCN(c4ncccc4OC)CC3)[nH]c2c1F]

In some embodiments, Ring W is:

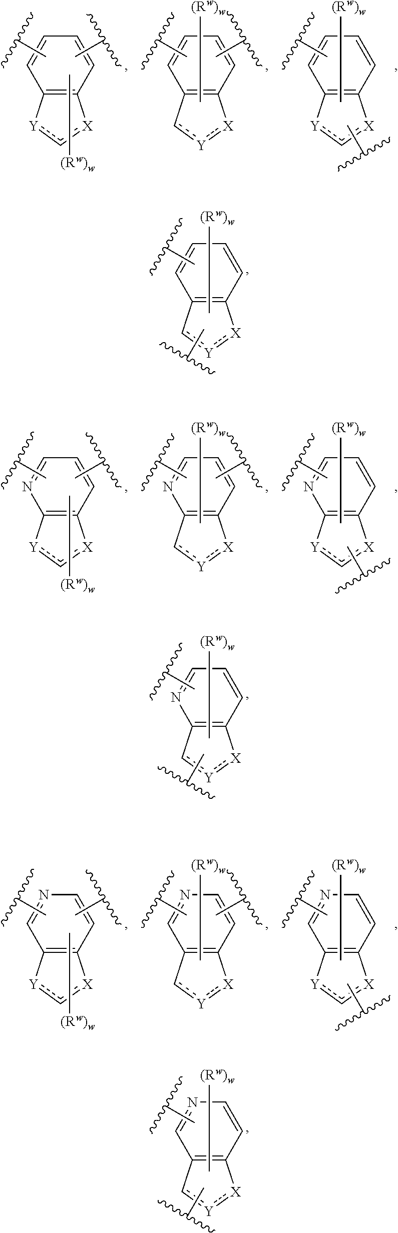

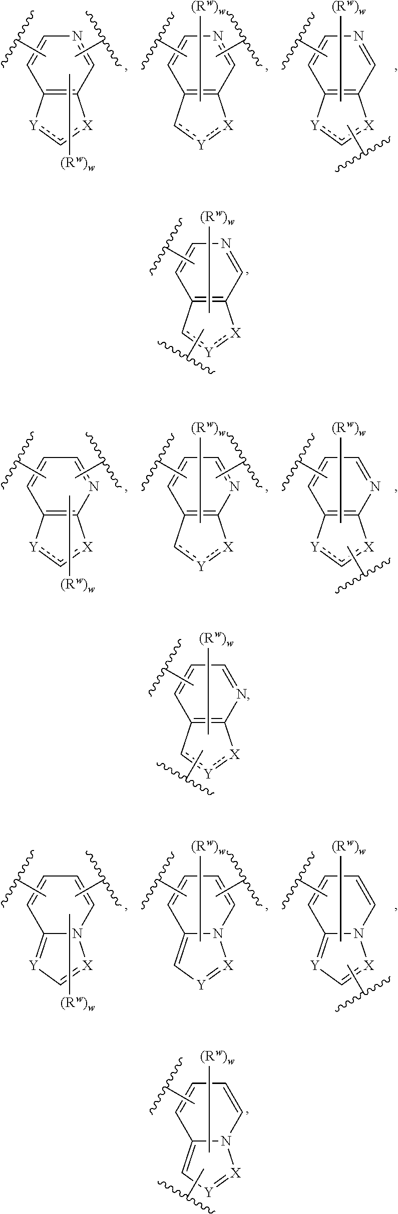

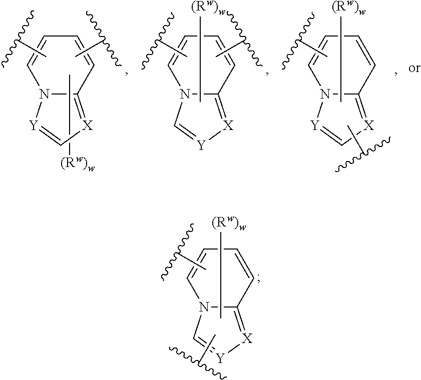

wherein R^{w} and w are as defined above and described herein; and
each of X and Y of Ring W is independently N, NH, N—R^{W}, —O—, —S—, C—H, C—R^{W}, C—H₂, CH(Wv), or C—(R^{W})₂.

In some embodiments, Ring W is

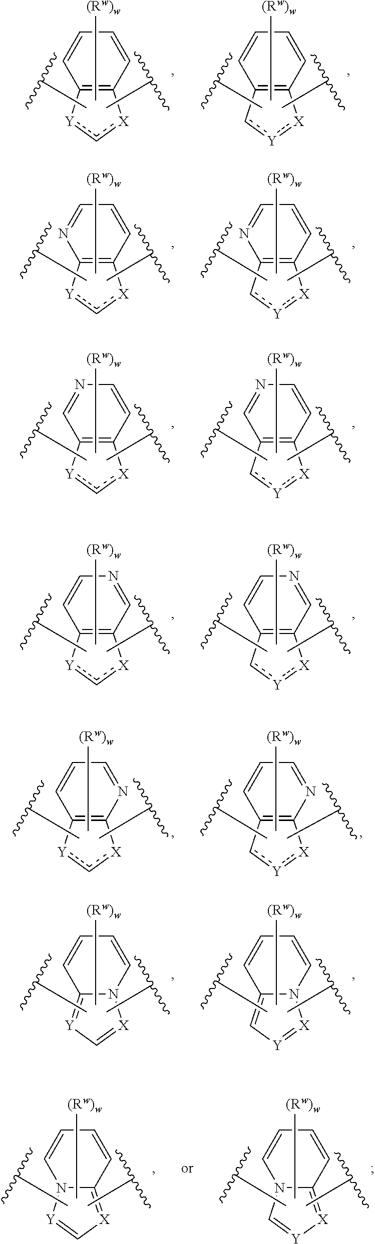

wherein each of R^{w}, w, X, and Y is as defined above and described here.

In some embodiments, Ring W is

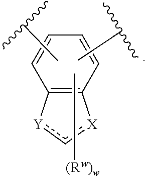

In some embodiments, Ring W is

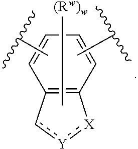

In some embodiments, Ring W is

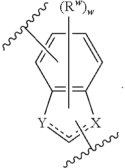

In some embodiments, Ring W is

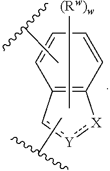

In some embodiments, Ring W is

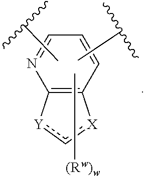

In some embodiments, Ring W is

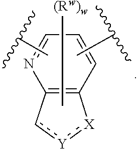

In some embodiments, Ring W is

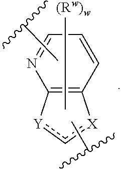

In some embodiments, Ring W is

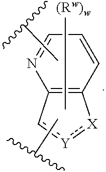

In some embodiments, Ring W is

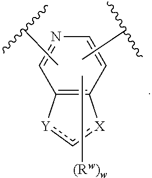

In some embodiments, Ring W is

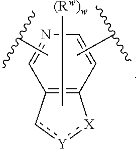

In some embodiments, Ring W is

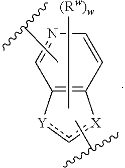

In some embodiments, Ring W is

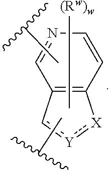

In some embodiments, Ring W is

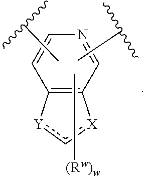

In some embodiments, Ring W is

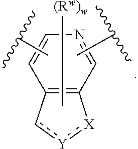

In some embodiments, Ring W is

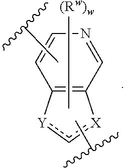

In some embodiments, Ring W is

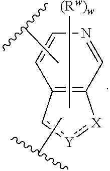

In some embodiments, Ring W is

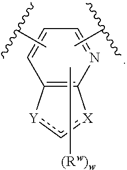

In some embodiments, Ring W is

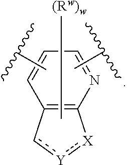

In some embodiments, Ring W is

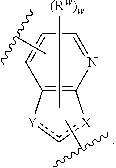

In some embodiments, Ring W is

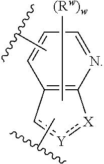

In some embodiments, Ring W is

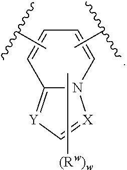

In some embodiments, Ring W is

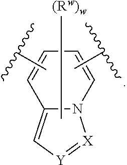

In some embodiments, Ring W is

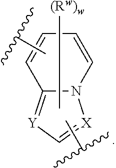

In some embodiments, Ring W is

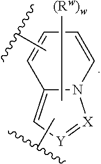

In some embodiments, Ring W is

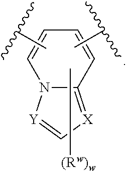

In some embodiments, Ring W is

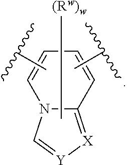

In some embodiments, Ring W is

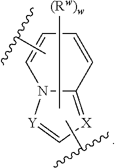

In some embodiments, Ring W is

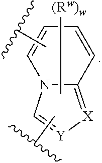

In some embodiments, each of X and Y of Ring W is independently N, NH, N—R^{W}, —O—, —S—, C—H, C—R^{W}, CH₂, CH(R^{W}), or C—(R^{w})₂.

In some embodiments, Ring W is

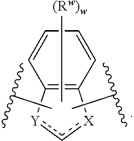

In some embodiments, Ring W is

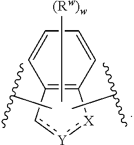

In some embodiments, Ring W is

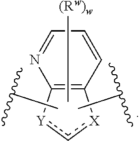

In some embodiments, Ring W is

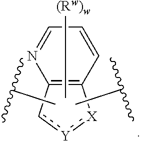

In some embodiments, Ring W is

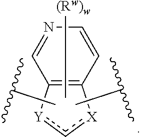

In some embodiments, Ring W is

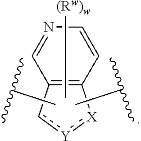

In some embodiments, Ring W is

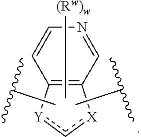

In some embodiments, Ring W is

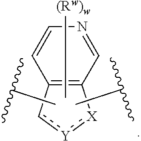

In some embodiments, Ring W is

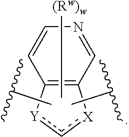

In some embodiments, Ring W is

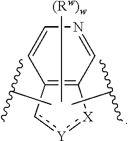

In some embodiments, Ring W is

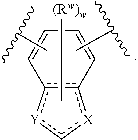

In some embodiments, Ring W is

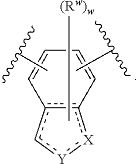

As defined above and described herein, each of X and Y of Ring W is independently N, NH, N—R^{W}, —O—, —S—, C—H, C—R^{W}, C—H₂, CH(R^{W}), or C—(R^{W})₂. In some embodiments, each of X and Y of Ring W is independently N, NH, N—R^{w}, —O—, —S—, C—H, C—R^{W}, C—H₂, CH(R^{W}), or C—(R^{W})₂, as allowed by valency.

In some embodiments, X is C—R^{w} or CH, and Y is N—R^{w}. In some embodiments, X is C—R^{w} or CH, and Y is S. In some embodiments, X is C—R^{w} or CH, and Y is O. In some embodiments, X is N—R^{w} or NH, and Y is C—R^{w} or CH. In some embodiments, X is S, and Y is C—R^{w} or CH. In some embodiments, X is O, and Y is C—R^{w} or CH.

In some embodiments, Ring W is

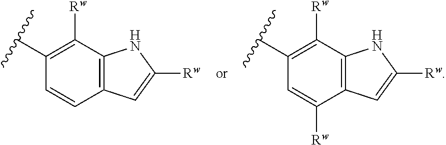

In some embodiments Ring W is

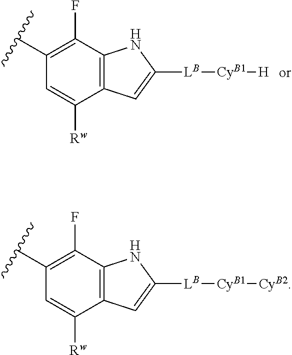

In some embodiments, Ring W is

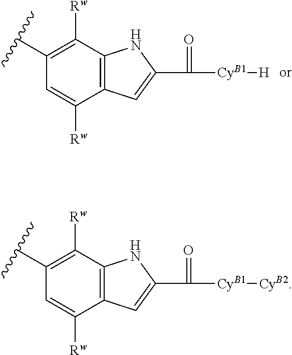

In some embodiments Ring W is

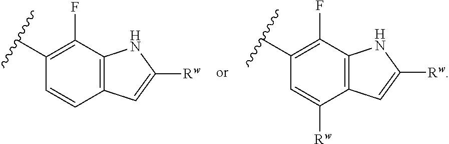

In some embodiments Ring W is

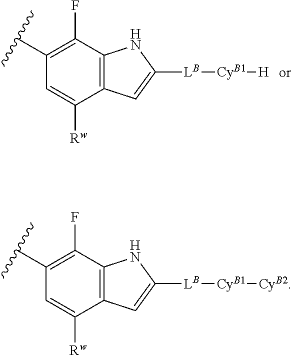

In some embodiments Ring W is

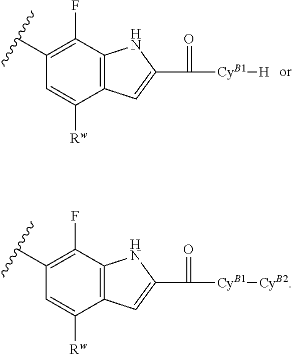

In some embodiments, Ring W is

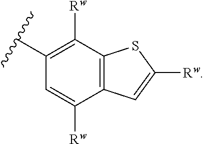

In some embodiments, Ring W is

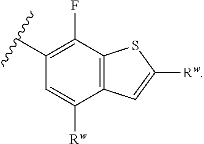

In some embodiments, Ring W is

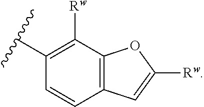

As described above and defined herein, Ring X is a ring selected from phenylenyl, 3-8 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen, and sulfur.

In some embodiments, Ring X is a ring selected from phenylenyl, 4-7 membered saturated or partially unsaturated carbocyclylenyl or heterocyclylenyl with 1-3 heteroatoms independently selected from nitrogen, oxygen, and sulfur, and 5-6 membered heteroarylenyl with 1-4 heteroatoms independently selected from nitrogen, oxygen, and sulfur.

In some embodiments, Ring X is a ring selected from a 3-8 membered saturated or partially unsaturated carbocyclylenyl or heterocyclylenyl with 1-3 heteroatoms independently selected from nitrogen, oxygen, and sulfur. In some embodiments, Ring X is a ring selected from a 3-8 membered saturated or partially unsaturated carbocyclylenyl. In some embodiments, Ring X is a ring selected from a 3-8 membered saturated or partially unsaturated heterocyclylenyl with 1-3 heteroatoms independently selected from nitrogen, oxygen, and sulfur.

In some embodiments, Ring X is phenylenyl. In some embodiments, Ring X is a 4-7 membered saturated or partially unsaturated carbocyclylenyl. In some embodiments, Ring X is a 4-7 membered saturated or partially unsaturated heterocyclylenyl with 1-3 heteroatoms independently selected from nitrogen, oxygen, and sulfur. In some embodiments, Ring X is a 5-6 membered heteroarylenyl with 1-4 heteroatoms independently selected from nitrogen, oxygen, and sulfur.

In some embodiments, Ring X is a 6-membered saturated or partially unsaturated heterocyclylenyl with 1-3 heteroatoms independently selected from nitrogen, oxygen, and sulfur. In some embodiments, Ring X is a 6-membered saturated or partially unsaturated heterocyclylenyl having 1 nitrogen atom.

In some embodiments, Ring X is a ring selected from a 5-6 membered saturated or partially unsaturated heterocyclylenyl with 1-3 heteroatoms independently selected from nitrogen, oxygen, and sulfur. In some embodiments, Ring X is a ring selected from a 6-7 membered saturated or partially unsaturated heterocyclylenyl with 1-3 heteroatoms independently selected from nitrogen, oxygen, and sulfur. In embodiments, Ring X is a 7-membered saturated or partially unsaturated heterocyclylenyl with 1-3 heteroatoms independently selected from nitrogen, oxygen, and sulfur. In some embodiments, Ring X is a 7-membered saturated or partially unsaturated heterocyclylenyl having 1 nitrogen atom.

In some embodiments, Ring X is

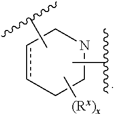

In some embodiments, Ring X is

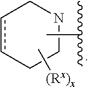

In some embodiments, Ring W is

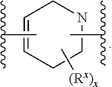

In some embodiments, Ring W is

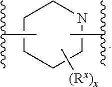

In some embodiments, Ring W is

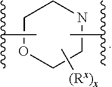

In some embodiments, Ring W is

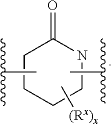

In some embodiments, Ring W is

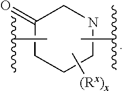

In some embodiments, Ring W is

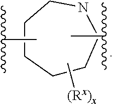

In some embodiments, Ring W is

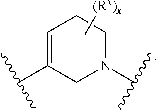

In some embodiments, Ring W is

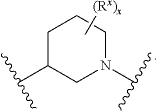

In some embodiments, Ring W is

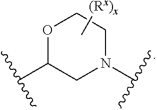

In some embodiments, Ring W is

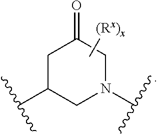

In some embodiments, Ring W is

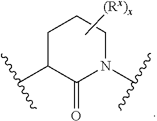

In some embodiments, Ring W is

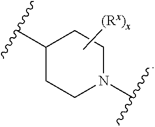

In some embodiments, Ring W is

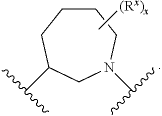

In some embodiments, Ring W is

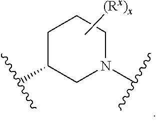

In some embodiments, Ring W is

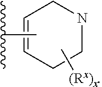

In some embodiments, Ring W is

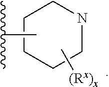

In some embodiments, Ring W is

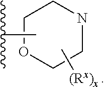

In some embodiments, Ring W is

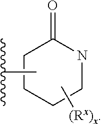

In some embodiments, Ring W is

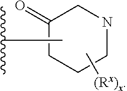

In some embodiments, Ring W is

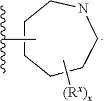

In some embodiments, Ring W is

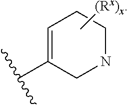

In some embodiments, Ring W is

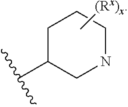

In some embodiments, Ring W is

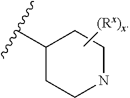

In some embodiments, Ring W is

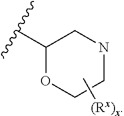

In some embodiments, Ring W is

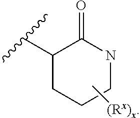

In some embodiments, Ring W is

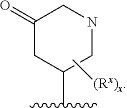

In some embodiments, Ring W is

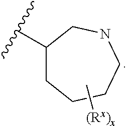

In some embodiment, Ring X is as depicted in the compounds of Table 1B below.

In some embodiments, Ring X and its R^{x} substituents are

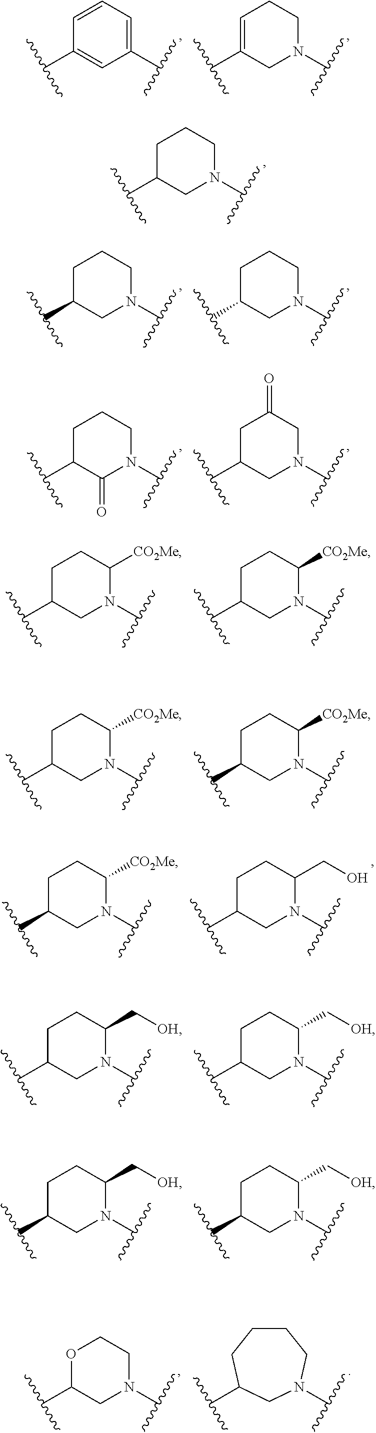

In some embodiments, Ring X and its R^{x} substituents are

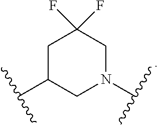 [SMILES: *C1CN(*)CC(F)(F)C1]

In some embodiments, Ring X and its R^{x} substituents are

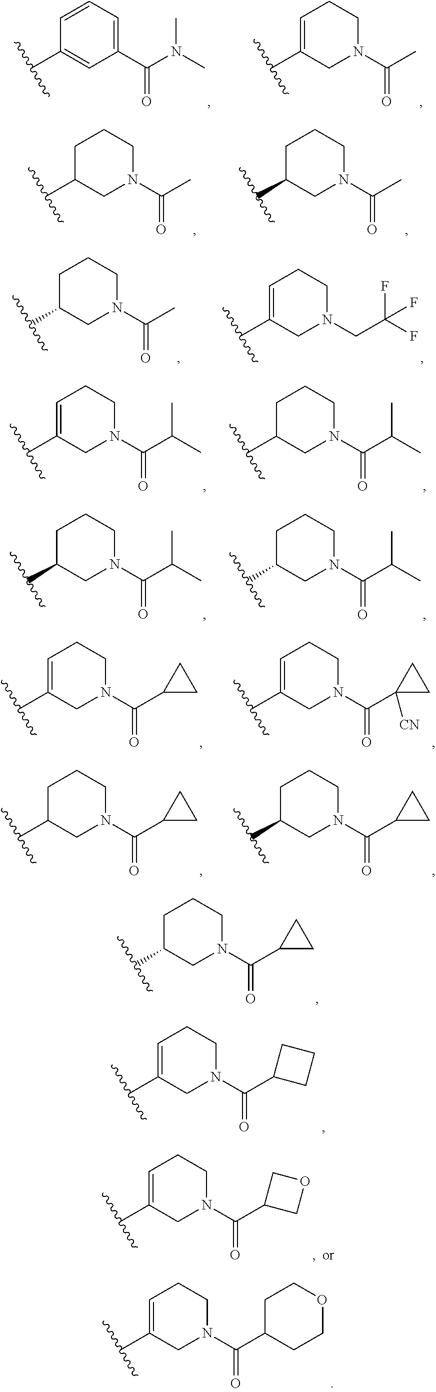

As described above and defined herein, G is hydrogen or

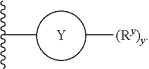

In some embodiments, G is hydrogen. In some embodiments, G is

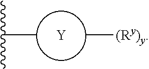

In some embodiment, G is as depicted in the compounds of Table 1B, below.

As described above and defined herein. Ring Y is a ring selected from phenyl, naphthyl, 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, and a 5-10 membered monocyclic or bicyclic heteroaryl with 1-4 heteroatoms independently selected from nitrogen, oxygen, and sulfur.

In some embodiments, Ring Y is phenyl. In some embodiments, Ring Y is naphthyl. In some embodiments, Ring Y is a 3-11 membered saturated or partially unsaturated monocyclic, bicyclic, bridged bicyclic, or spirocyclic carbocyclyl. In some embodiments, Ring Y is a 5-11 membered saturated or partially unsaturated monocyclic, bicyclic, bridged bicyclic, or spirocyclic heterocyclyl with 1-3 heteroatoms independently selected from nitrogen, oxygen, and sulfur. In some embodiments, Ring Y is a 5-10 membered monocyclic or bicyclic heteroaryl with 1-4 heteroatoms independently selected from nitrogen, oxygen, and sulfur.

In some embodiments, Ring Y is a 3-8 membered saturated or partially unsaturated monocyclic carbocyclyl. In some embodiments, Ring Y is a 5-6 membered saturated or partially unsaturated monocyclic carbocyclyl. In some embodiments, Ring Y is a 6-7 membered saturated or partially unsaturated monocyclic carbocyclyl. In some embodiments, Ring Y is a 3 membered saturated or partially unsaturated monocyclic carbocyclyl. In some embodiments, Ring Y is a 4 membered saturated or partially unsaturated monocyclic carbocyclyl. In some embodiments, Ring Y is a 5 membered saturated or partially unsaturated monocyclic carbocyclyl. In some embodiments, Ring Y is a 6 membered saturated or partially unsaturated monocyclic carbocyclyl. In some embodiments, Ring Y is a 7 membered saturated or partially unsaturated monocyclic carbocyclyl. In some embodiments, Ring Y is cyclopropyl, cyclobutyl, cyclopentyl, cyclohexyl, or cycloheptyl.

In some embodiments, Ring Y is a 3-6 membered saturated or partially unsaturated monocyclic carbocyclyl. In some embodiments, Ring Y is cyclopropyl. In some embodiments, Ring Y is cyclobutyl. In some embodiments, Ring Y is cyclopentyl. In some embodiments, Ring Y is hexyl.

In some embodiments, Ring Y is a 5-6 membered monocyclic heteroaryl with 1-4 heteroatoms independently selected from nitrogen, oxygen, and sulfur. In some embodiments, Ring Y is a 9-membered bicyclic heteroaryl with 1-4 heteroatoms independently selected from nitrogen, oxygen, and sulfur. In some embodiments, Ring Y is a 5-membered monocyclic heteroaryl with 1-4 heteroatoms independently selected from nitrogen, oxygen, and sulfur.

In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 4 heteroatoms independently selected from nitrogen, oxygen, and sulfur. In some embodiments, Ring Y is a 5-membered monocyclic heteroaryl ring with 2-3 nitrogen heteroatoms.

In some embodiments, Ring Y is a 6-membered monocyclic heteroaryl with 1-4 heteroatoms independently selected from nitrogen, oxygen, and sulfur. In some embodiments, Ring Y is a 6-membered monocyclic heteroaryl with 1-4 nitrogen heteroatoms. In some embodiments, Ring Y is pyridinyl, pyrimidinyl, pyridazinyl, or triazinyl.

In some embodiments, Ring Y is an 8-11 membered saturated or partially unsaturated bicyclic, bridged bicyclic, or spirocyclic heterocyclyl with 1-3 heteroatoms independently selected from nitrogen, oxygen, and sulfur. In some embodiments, Ring Y is a 8-11 membered saturated or partially unsaturated bicyclic heterocyclyl with 1-3 heteroatoms independently selected from nitrogen, oxygen, and sulfur. In some embodiments, Ring Y is a 9-10 membered saturated or partially unsaturated bicyclic heterocyclyl with 1-3 heteroatoms independently selected form nitrogen, oxygen, and sulfur. In some embodiments, Ring Y is a 9 membered saturated or partially unsaturated bicyclic heterocyclyl with 1-3 heteroatoms independently selected from nitrogen, oxygen, and sulfur. In some embodiments, Ring Y is a 10 membered saturated or partially unsaturated bicyclic heterocyclyl with 1-3 heteroatoms independently selected from nitrogen, oxygen, and sulfur. In some embodiments, Ring Y is tetrahydroisoquinolinyl or tetrahydroquinolinyl.

In some embodiments, Ring Y is a 9-10 membered bicyclic heteroaryl with 1-4 heteroatoms independently selected from nitrogen, oxygen, and sulfur. In some embodiments, Ring Y is a 9 membered bicyclic heteroaryl with 1-4 heteroatoms independently selected from nitrogen, oxygen, and sulfur. In some embodiments, Ring Y is a 9 membered bicyclic heteroaryl with 1-4 nitrogen heteroatoms.

In some embodiments, Ring Y is

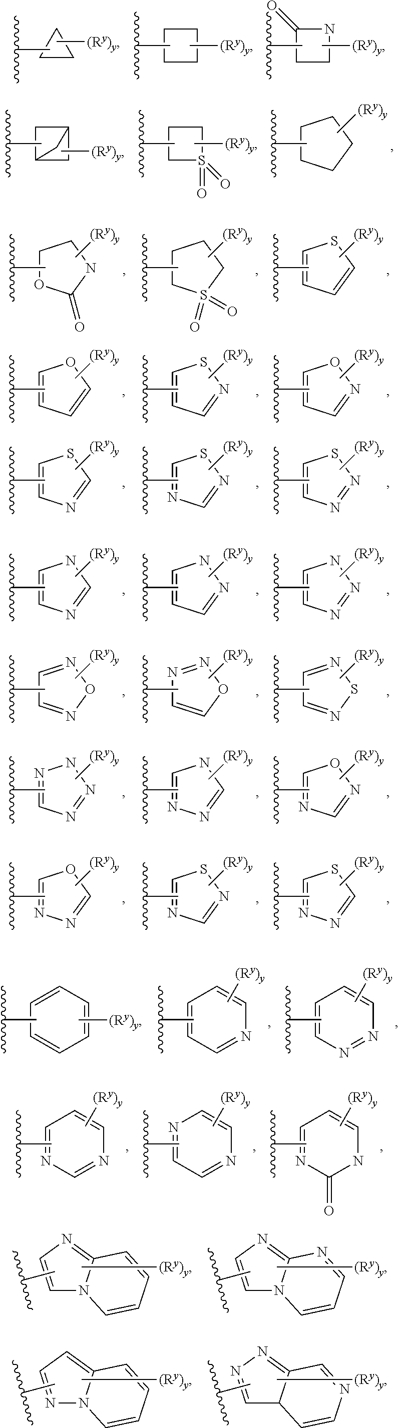

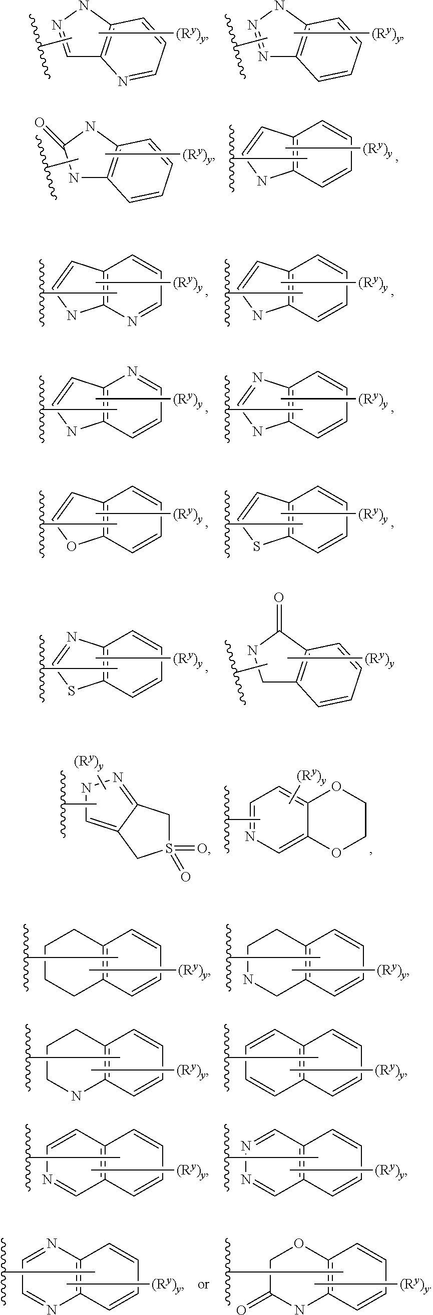

In some embodiments, Ring Y is

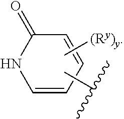

In some embodiments, Ring Y is

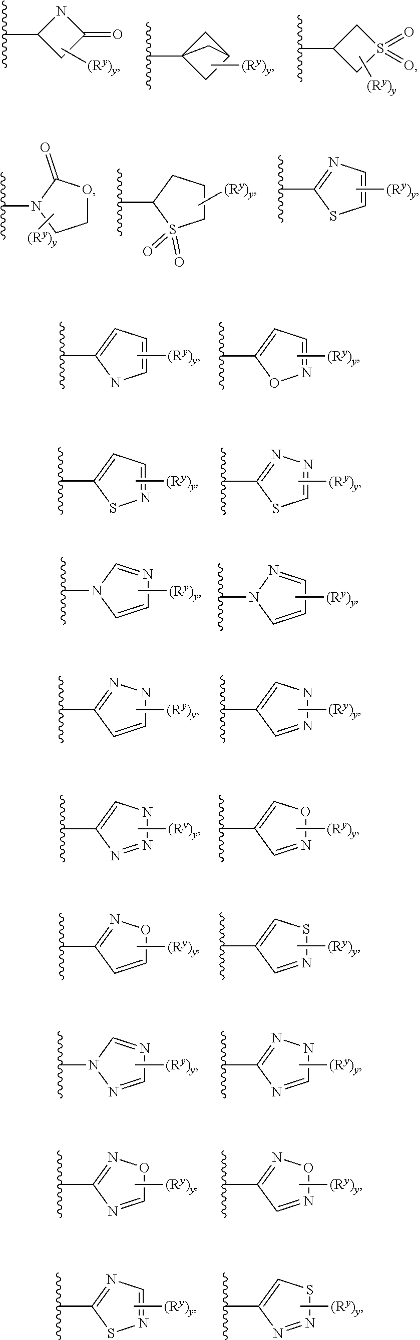

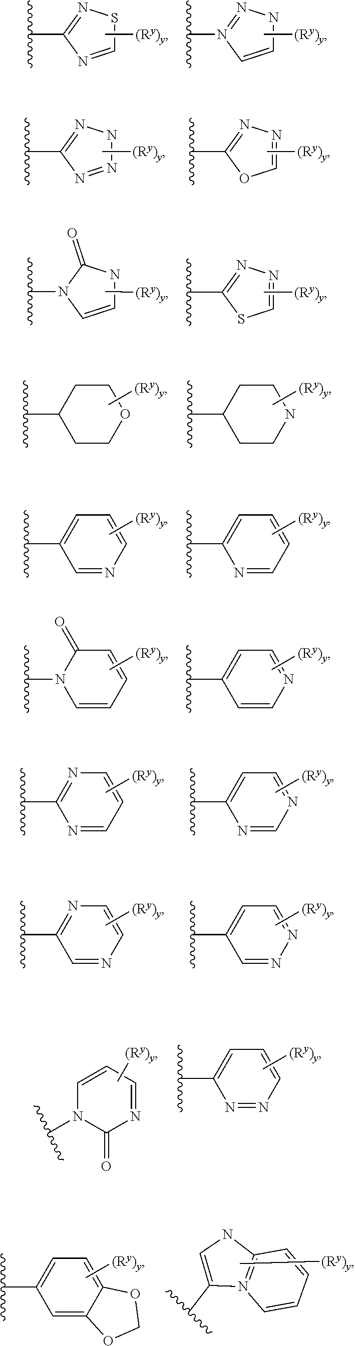

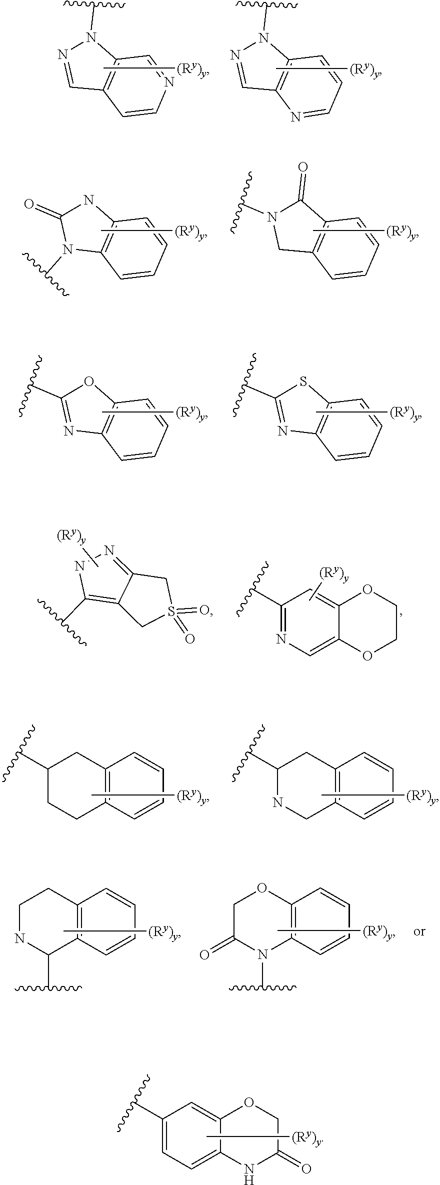

In some embodiment, Ring Y is

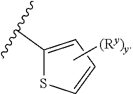

In some embodiment, Ring Y is as depicted in the compounds of Table 1B, below.

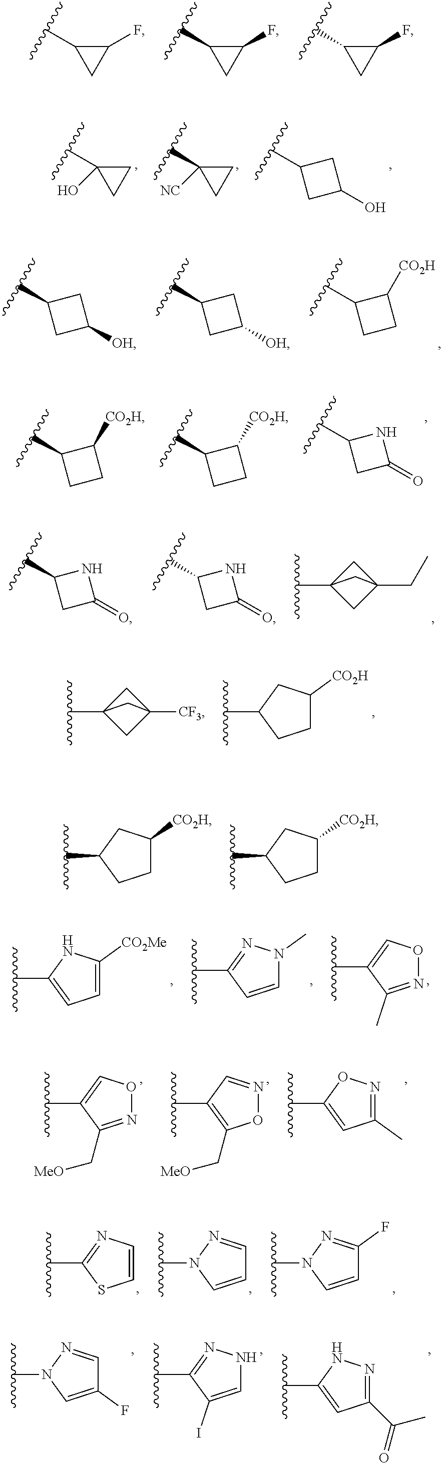

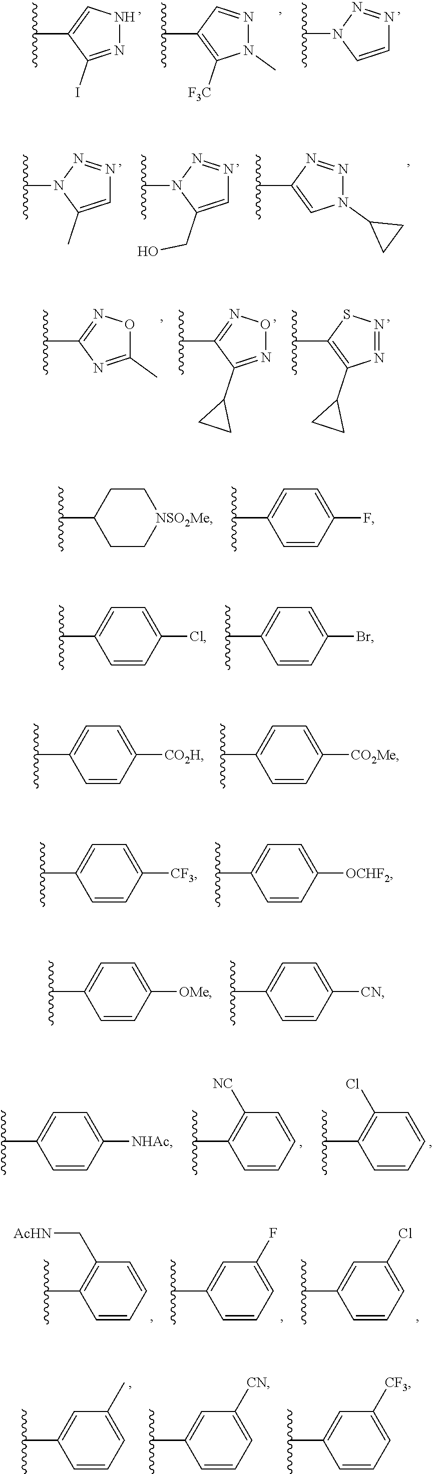

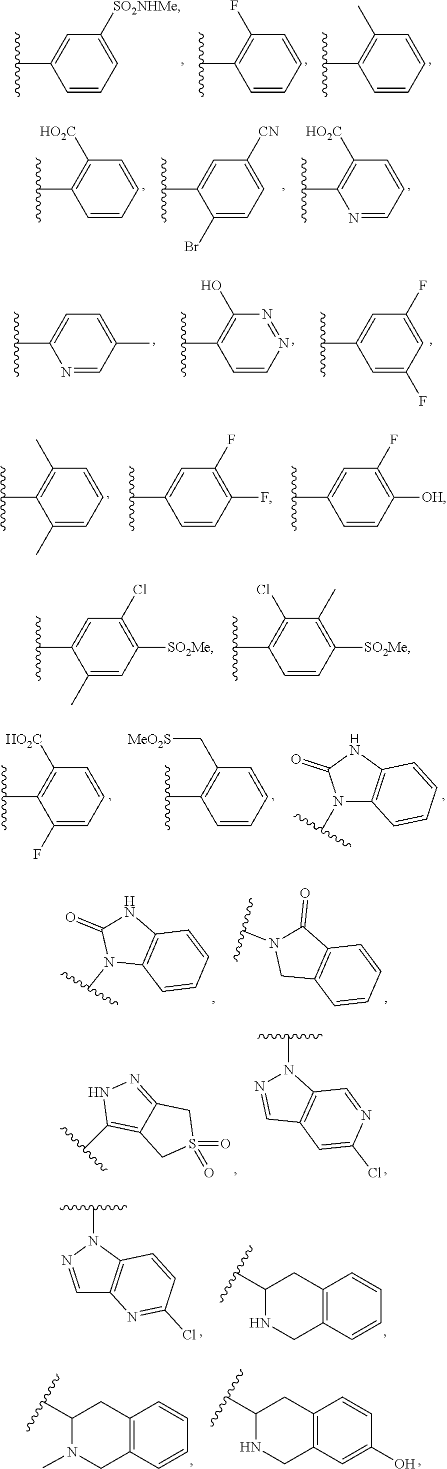

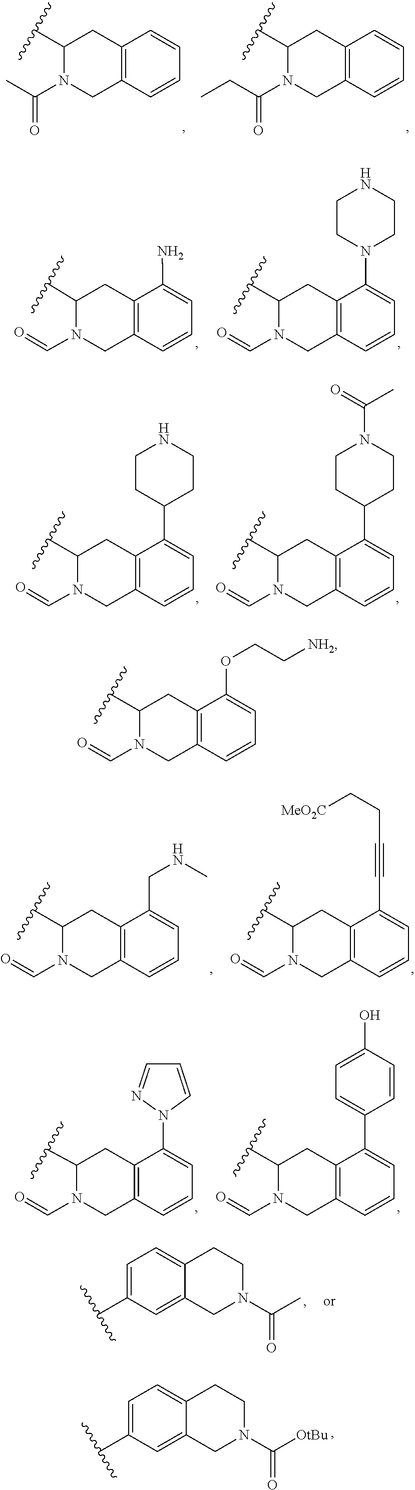

In some embodiments, Ring Y and its R^{y} substituents are

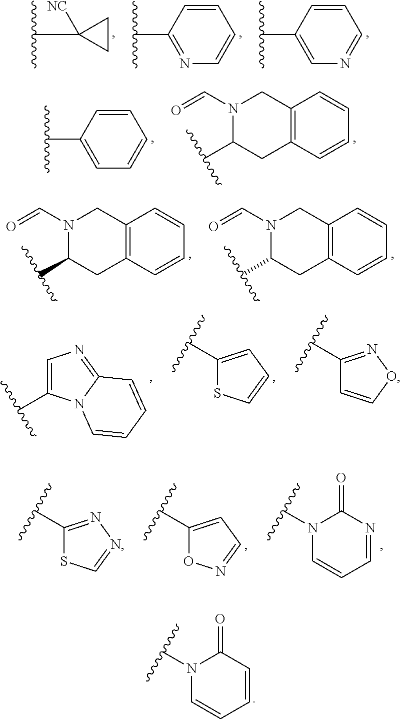

In some embodiments, Ring Y is

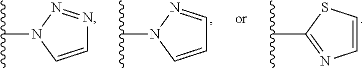

As described above and defined herein, R^{x} and R^{y} are independently selected from hydrogen, R^{A}, halogen, —CN, —NO₂, —OR—SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R¹ is independently selected from hydrogen, R^{A}, R^{B}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from hydrogen, R^{A}, halogen, —CN, —NO₂. —OR—SR, —NR₂, —SiR₃, —S(O)R, —S(O)₂R, —S(O)(NR)R—S(O)₂NR₂—S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from R^{A}, R^{B}, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{w} is hydrogen. In some embodiments, R^{w} is R^{A}. In some embodiments, R^{w} is halogen. In some embodiments, R^{w} is —CN. In some embodiments, R^{w} is —NO₂. In some embodiments, R^{w} is —OR. In some embodiments, R^{w} is —SR. In some embodiments, R^{w} is —NR₂. In some embodiments, R^{w} is —SiR₃. In some embodiments, R^{w} is —S(O)₂R. In some embodiments, R^{w} is —S(O)₂NR₂. In some embodiments, R^{w} is —S(O)(NR)R. In some embodiments, R^{w} is —S(O)R. In some embodiments, R^{w} is —C(O)R. In some embodiments, R^{w} is —C(O)OR. In some embodiments, R^{w} is —C(O)NR₂. In some embodiments, R^{w} is —C(O)NROR. In some embodiments, R^{w} is —OC(O)R. In some embodiments, R^{w} is —OC(O)NR₂. In some embodiments, R^{w} is —P(O)R₂. In some embodiments, R^{w} is —P(O)(OR)₂. In some embodiments, R^{W} is —OP(O)R₂. In some embodiments, R^{w} is —OP(O)(OR)₂. In some embodiments, R^{w} is —OP(O)(OR)NR₂. In some embodiments, R is —OP(O)(NR₂)₂. In some embodiments, R^{w} is —NRC(O)OR. In some embodiments, R^{W} is —NRC(O)R. In some embodiments, R^{W} is —NRC(O)N(R)₂. In some embodiments, R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w}—C(O)OR. In some embodiments, R^{w} is —C(O)NR₂. In some embodiments, R^{W} is an optionally substituted phenyl. In some embodiments, R^{W} is an optionally substituted 5-6 membered heteroaryl ring having 1-4 heteroatoms independently selected from nitrogen, oxygen, and sulfur.

In some embodiments, R^{w} is fluoro, chloro, or bromo.

In some embodiments, R^{w} is —C(O)NHR. In some embodiments, R^{w} is —C(O)NHR wherein R of R^{w} is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{W} is optionally substituted C₁₋₆ aliphatic. In some embodiments, R^{w} is C₁₋₆ aliphatic, optionally substituted with —C(O)N(R^{o})₂. In some embodiments, R^{w} is C₁₋₆ aliphatic, optionally substituted with —NR^{o}(O)N(R^{o})₂. In some embodiments, R^{w} is

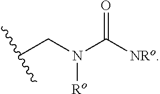

In some such embodiments, R^{o} is hydrogen or C₁₋₆ aliphatic. In some embodiments, R^{w} is C₁₋₆ aliphatic optionally substituted with —OR^{o}, wherein R^{o} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, Rh is C₁₋₆ aliphatic optionally substituted with halogen (e.g., fluoro). In some embodiments, R^{w} is

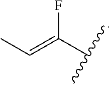 [SMILES: */C(F)=C/C]

In some embodiments, R^{w} is C₁₋₆ aliphatic optionally substituted with halogen (e.g., fluoro) or —NR^{o}₂. In some embodiments, R^{W} is C₁₋₆ aliphatic optionally substituted with halogen (e.g., fluoro) and —NR^{o}₂. In some embodiments, R^{W} is C₁₋₆ aliphatic optionally substituted with halogen (e.g., fluoro) or —NR^{o}₂, wherein each R^{o} is independently hydrogen or C₁₋₆ aliphatic. In some embodiments, R^{W} is C₁₋₆ aliphatic optionally substituted with halogen (e.g., fluoro) and —NR^{o}₂, wherein each R^{o} is independently hydrogen or C₁₋₆ aliphatic. In some embodiments, R^{w} is

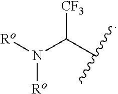

In some embodiments, R^{w} is C₁₋₆ aliphatic optionally substituted with (CH₂)₀₋₄R^{o} wherein R^{o} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —(CH₂)₀₋₄R^{o}, wherein R^{o} is a C₁₋₆ aliphatic. In some embodiments, R^{W} is C₆ aliphatic optionally substituted with —R^{o}, wherein R^{o} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —R^{o}, wherein R^{o} is a C₁₋₆ aliphatic.

In some embodiments, R^{w} is C₁₋₆ aliphatic optionally substituted with —(CH₂)₀₋₄R^{o}, wherein R^{o} is cyclopropyl, cyclobutyl, cyclopentyl, or cyclohexyl. In some embodiments, R^{w} is C₁₋₆ aliphatic optionally substituted with —R^{o}, wherein R^{o} is a cyclopropyl, cyclobutyl, cyclopentyl, or cyclohexyl.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 5-6 membered saturated or partially unsaturated carbocyclic or heterocyclic ring having 1-2 heteroatoms independently selected from nitrogen, oxygen, and sulfur.

In some embodiments R^{w} is an optionally substituted phenyl.

In some embodiments R^{w} is an optionally substituted 3-7 membered saturated or partially unsaturated carbocyclic. In some embodiments R^{w} is an optionally substituted 3-7 membered saturated or partially unsaturated carbocyclic. In some embodiments R^{w} is an optionally substituted cyclopropyl. In some embodiments R^{w} is an optionally substituted cyclobutyl. In some embodiments R^{w} is an optionally substituted cyclopentyl. In some embodiments R^{w} is an optionally substituted cyclohexyl. In some embodiments R^{w} is an optionally substituted cyclopropenyl. In some embodiments R^{w} is an optionally substituted cyclobutenyl. In some embodiments R^{w} is an optionally substituted cyclopentenyl. In some embodiments R^{w} is an optionally substituted cyclohexenyl.

In some embodiments R^{w} is an optionally substituted 3-7 membered saturated or partially unsaturated heterocyclic ring having 1-2 heteroatoms independently selected from nitrogen, oxygen, and sulfur. In some embodiments R^{w} is an optionally substituted 5-6 membered saturated or partially unsaturated heterocyclic ring having 1-2 heteroatoms independently selected from nitrogen, oxygen, and sulfur.

In some embodiments R^{w} is an optionally substituted 4 membered saturated or partially unsaturated heterocyclic ring having 1-2 heteroatoms independently selected from nitrogen, oxygen, and sulfur. In some embodiments R^{w} is an optionally substituted azetidinyl, oxetanyl, or thietanyl.

In some embodiments R^{w} is an optionally substituted 5 membered saturated or partially unsaturated heterocyclic ring having 1-2 heteroatoms independently selected from nitrogen, oxygen, and sulfur. In some embodiments, R^{w} is an optionally substituted pyrrolidinyl, pyrrolinyl, pyrazolidinyl, pyrazolinyl, imidazolidinyl, or imidazolinyl. In some embodiments, R^{w} is an optionally substituted dihydropyridinyl, pyrrolidinyl, dihydrofuranyl, tetrahydrofuranyl, dihydrothiophenyl, or tetrahydrothiophenyl.

In some embodiments R^{w} is an optionally substituted 6 membered saturated or partially unsaturated heterocyclic ring having 1-2 heteroatoms independently selected from nitrogen, oxygen, and sulfur. In some embodiments R^{W} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithianyl, 1,4-dithianyl, morpholinyl, or thiomorpholinyl. In some embodiments, R^{w} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w} is an optionally substituted

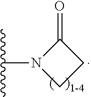

In some embodiments, R^{w} is an optionally substituted

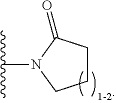

In some embodiments, R^{w} is an optionally substituted

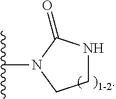

In some embodiments, R^{w} is

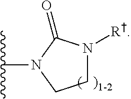

In some embodiments, R^{w} is optionally substituted

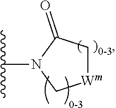

where W^{m} is O, S, C(O), or NR^{o}, wherein R^{o} is as described above and defined herein. In some embodiments, R^{w} is

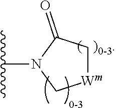

In some embodiments, R^{w} is optionally substituted

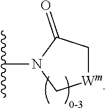

In some embodiments, R¹ is

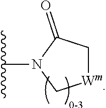

In some embodiments, R^{w} is

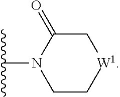

In some embodiments, R^{w} is

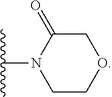 [SMILES: *N1CCOCC1=O]

In some embodiments, W^{m} is O. In some embodiments, W^{m} is S. In some embodiments, W^{m} is C(O). In some embodiments, W^{m} is NR^{o}. In some embodiments, W^{m} is NR^{o}, wherein R^{o} is hydrogen or C₁₋₆ aliphatic.

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{o})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{o})₂. †

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{w} is optionally substituted pyridinyl, pyrimidinyl, pyridazinyl, or triazinyl. In some embodiments, R^{w} is an optionally substituted pyridinonyl, pyrazinonyl, or pyrimidinonyl.

In some embodiments, R^{w} is —NHR wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In some embodiments, R^{w} is —S(O)₂NH₂. In some embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)H. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w} is —C(S)R, —C(NR)R, —S(O)R, —S(O)₂R, —S(O)(NR)R—S(O)₂NR₂. —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —S(O)R, or —NRS(O)₂R. In some embodiments, R^{W} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R—S(O)₂NR₂, —S(O)R, —C(S)R, —C(NR)R, —C(O)R, —C(O)OR, —C(O)NR₂, or —C(O)NROR. In some embodiments, R^{W} is —C(S)R, —C(NR)R, —C(O)R, —C(O)OR, or —C(O)NR₂.

In some embodiments, R^{w} is —C(O)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w} is —C(S)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OH. In some embodiments, R^{w} is —C(O)OR, wherein R is of R^{w} is optionally substituted C₁₋₆ aliphatic. In some embodiments, R^{w} is —C(O)OR, wherein R is of R^{W} is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NH₂. In some embodiments, R^{w} is —C(O)NR₂, wherein each R is of R is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{W} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(NR)R. In some embodiments, R^{w} is —C(NH)R. In some embodiments, R^{w} is C(NR)R, wherein each R is of R^{w} is independently hydrogen or an optionally substituted C₁₋₆ aliphatic. In some embodiments, R^{w} is C(NR)R, wherein each R is of R^{w} is independently hydrogen or an optionally substituted phenyl. In some embodiments, R^{w} is C(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is C(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{W} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R^{w} is —C(O)NR₂, wherein the two R groups of R^{w} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{W} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)H. In some embodiments, R^{w} is —OC(O)R, wherein R is of R^{w} is optionally substituted C₁₋₆ aliphatic. In some embodiments, R^{w} is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R¹ is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{W} is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{W} is independently hydrogen or an optionally substituted 5-6 membered heteroaryl ring having 1-4 heteroatoms independently selected from nitrogen, oxygen, and sulfur.

In some embodiments, R¹ is —NRC(O)N(R)₂, wherein each R is of R^{w} is independently hydrogen or an optionally substituted C₁₋₆ aliphatic. In some embodiments, R^{W} is —NRC(O)N(R)₂, wherein each R is of R^{W} is independently hydrogen or an optionally substituted phenyl. In some embodiments, R^{W} is —NRC(O)N(R)₂, wherein each R is of R^{W} is independently hydrogen or an optionally substituted 4-7 membered saturated or partially unsaturated heterocyclic having 1-2 heteroatoms independently selected from nitrogen, oxygen, and sulfur. In some embodiments, R^{W} is —NRC(O)N(R)₂, wherein each R is of R^{W} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{W} is independently hydrogen or an optionally substituted C₁₋₆ aliphatic. In some embodiments, R^{W} is —NRS(O)₂R, wherein each R is of R^{w} is independently hydrogen or an optionally substituted phenyl. In some embodiments, R^{w} is —NRS(O)₂R, wherein each R is of R^{W} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)H. In some embodiments, R^{w} is —S(O)R, wherein R is of R^{W} is optionally substituted C₁₋₆ aliphatic. In some embodiments, R^{W} is —S(O)R, wherein R is of R^{w} is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{W} is an optionally substituted 5-6 membered heteroaryl ring having 1-4 heteroatoms independently selected from nitrogen, oxygen, and sulfur.

In some embodiments, R^{w} is —S(O)₂H. In some embodiments, R^{w} is —S(O)₂R, wherein R is of R^{W} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R^{w} is independently hydrogen or optionally substituted phenyl. In some embodiments, R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, RV is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

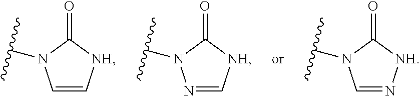 [SMILES: *n1cc[nH]c1=O ; *n1nc[nH]c1=O ; "or" ; *n1cn[nH]c1=O]

In some embodiments, R^{w} is:

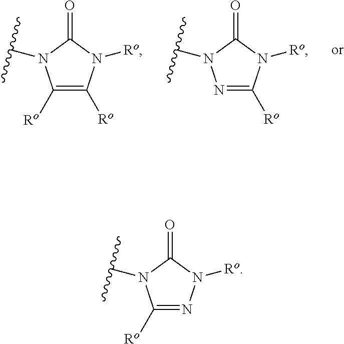

In some such embodiments, R^{o} is hydrogen or C₁₋₆ aliphatic. In some embodiments, R^{w} is:

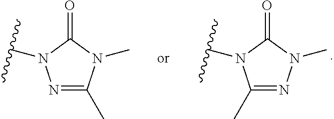

In some embodiments, R^{w} is an optionally substituted:

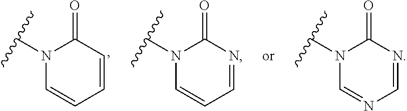

In some embodiments, R^{w} is:

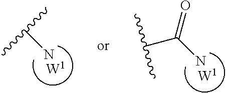

wherein Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.

As defined above and described herein, Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur. In some embodiments, Ring W¹ is an optionally substituted 3-7 membered saturated or partially unsaturated heterocyclic ring having 1 additional heteroatom selected from nitrogen, oxygen, and sulfur. In some embodiments, Ring W¹ is an optionally substituted 5-6 membered saturated or partially unsaturated heterocyclic ring having 1 additional heteroatom selected from nitrogen, oxygen, and sulfur. In some embodiments, Ring W¹ is an optionally substituted 5-6 membered monocyclic heteroaryl ring having 1-3 additional heteroatoms independently selected from nitrogen, oxygen, and sulfur. In some embodiments, Ring W¹ is an optionally substituted 5 membered monocyclic heteroaryl ring having 1-3 additional heteroatoms independently selected from nitrogen, oxygen, and sulfur. In some embodiments, Ring W¹ is an optionally substituted 6 membered monocyclic heteroaryl ring having 1-3 additional heteroatoms independently selected from nitrogen, oxygen, and sulfur.

In some embodiments, R^{w} is:

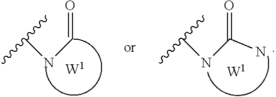

In some embodiments, R^{w} is:

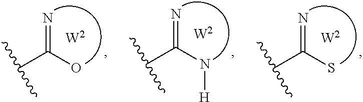

wherein Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

As defined above and described herein, Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur. In some embodiments, Ring W² is an optionally substituted 3-7 membered partially unsaturated heterocyclic ring. In some embodiments, Ring W² is an optionally substituted 5-6 membered partially unsaturated heterocyclic ring. In some embodiments, Ring W² is an optionally substituted 5-6 membered monocyclic heteroaryl ring having 1-2 additional heteroatoms independently selected from nitrogen, oxygen, and sulfur. In some embodiments, Ring W² is a 5 membered monocyclic heteroaryl ring having 1-2 additional heteroatoms independently selected from nitrogen, oxygen, and sulfur. In some embodiments, Ring W² is an optionally substituted oxazolyl, imidazolyl, thiazolyl, 1,3,4-thiadiazolyl, 2-imidazolinyl, 1,2,4-triazolyl, 1,2,4-oxadiazolyl, or 1,3,4-oxadiazolyl.

In some embodiments, R^{W} is an optionally substituted ring selected from:

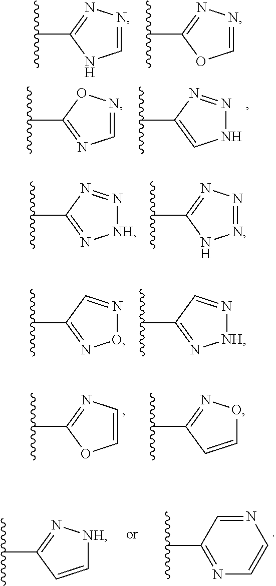

In some embodiments, R^{w} is:

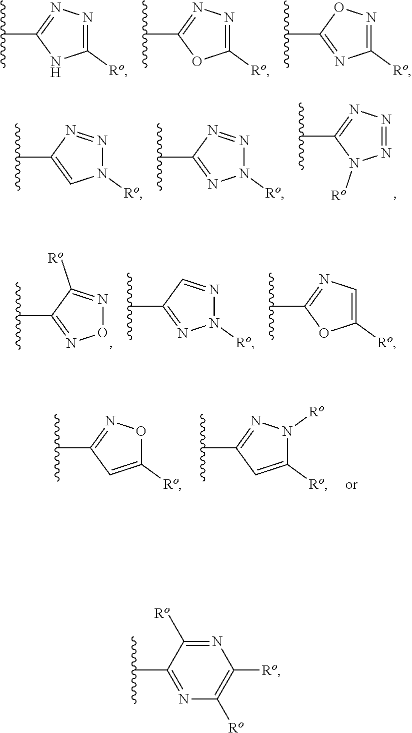

wherein each R^{o} is a defined above and described herein (e.g., hydrogen or C₁₋₆ aliphatic.

In some embodiments, R^{w} is:

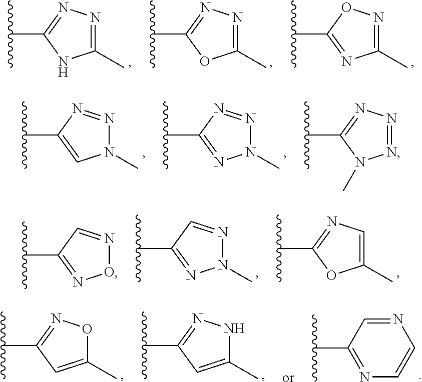

In some embodiments R^{W} is an optionally substituted 7-11 membered bicyclic heterocyclic ring having 1-4 heteroatoms independently selected from nitrogen, oxygen, and sulfur. In some embodiments R^{W} is an optionally substituted 9-10 membered bicyclic heterocyclic ring having 1-4 heteroatoms independently selected from nitrogen, oxygen, and sulfur. In some embodiments R^{w} is an optionally substituted benzo[d][1,3]dioxolyl. In some embodiments R^{w} is an optionally substituted an 11 membered bicyclic heterocyclic ring having 1-4 heteroatoms independently selected from nitrogen, oxygen, and sulfur.

In some embodiments R^{w} is an optionally substituted 8-11 membered bicyclic aryl or heteroaryl having 1-4 heteroatoms independently selected from nitrogen, oxygen and sulfur. In some embodiments, R^{w} is an optionally substituted naphthalenyl. In some embodiments, R^{W} is an optionally substituted dihydrobenzodioxepinyl. In some embodiments, R^{w} is an optionally substituted indenyl or dihydroindenyl.

In some embodiments, R^{w} is an optionally substituted 9-10 bicyclic heteroaryl having 1-4 heteroatoms independently selected from nitrogen, oxygen and sulfur.

In some embodiments R^{w} is an optionally substituted 9 bicyclic heteroaryl having 1-4 heteroatoms independently selected from nitrogen, oxygen and sulfur. In some embodiments R^{w} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments R^{w} is an optionally substituted 10 bicyclic heteroaryl having 1-4 heteroatoms independently selected from nitrogen, oxygen and sulfur. In some embodiments R^{w} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w} is R^{B}.

In some embodiments, R^{w} is fluoro, chloro, —CN, methyl, —CF₃, —CHF₂, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

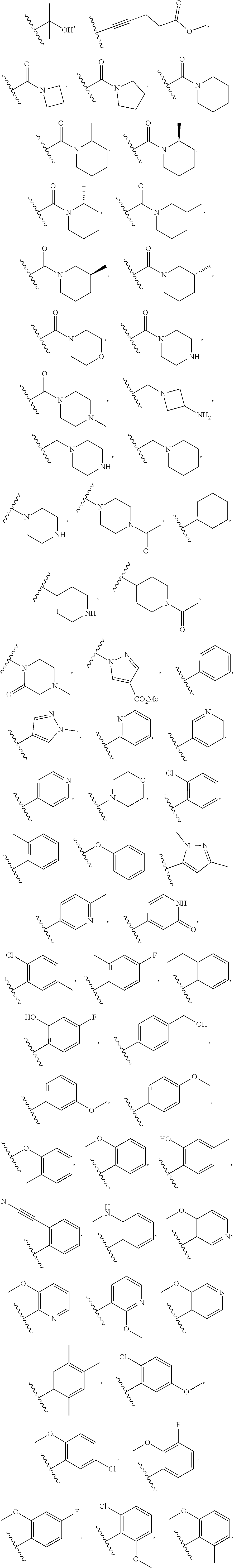

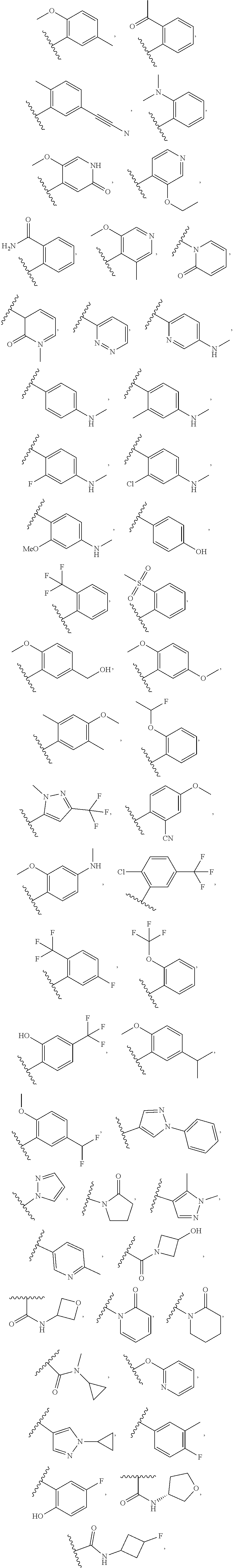

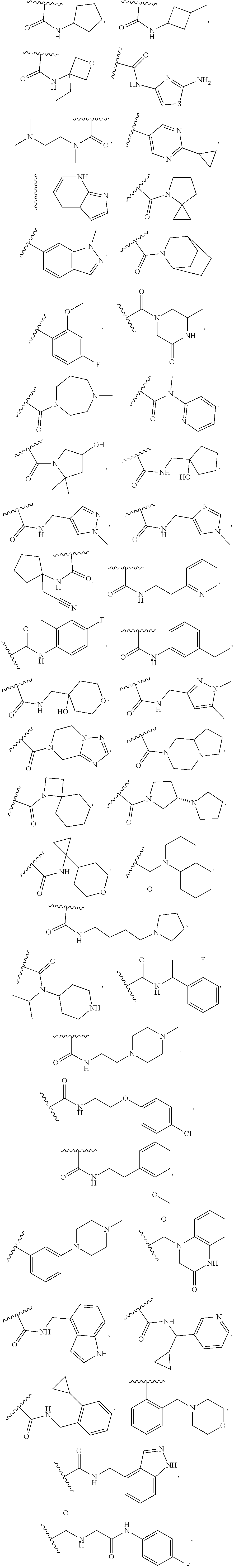

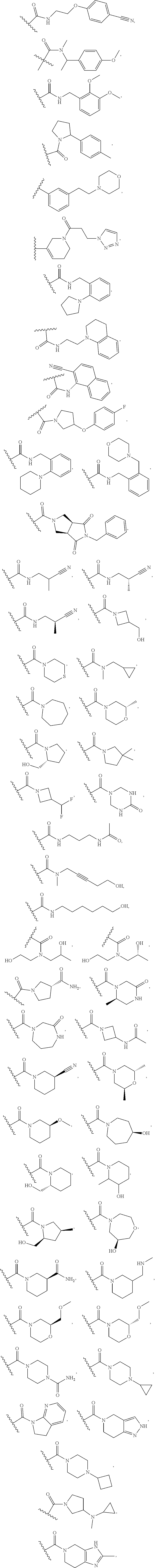

[chemical structure]

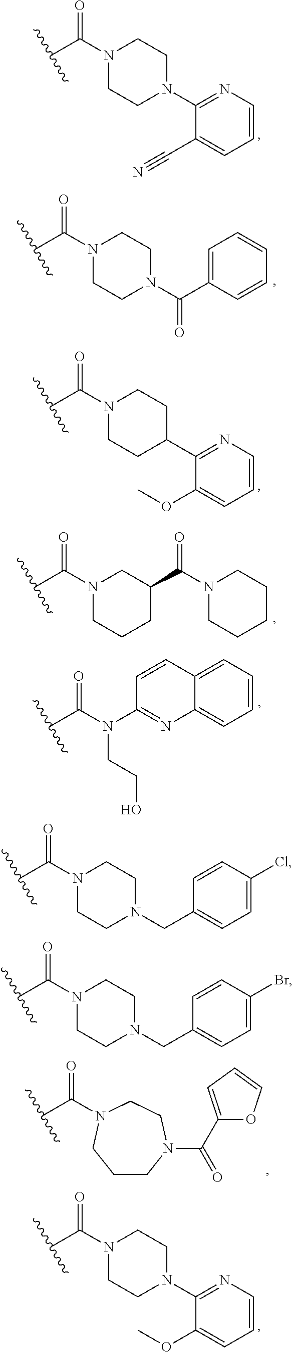

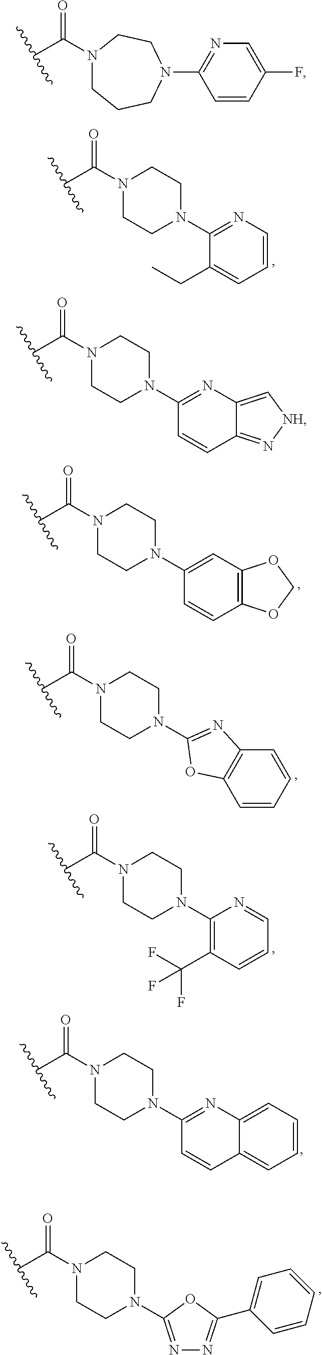

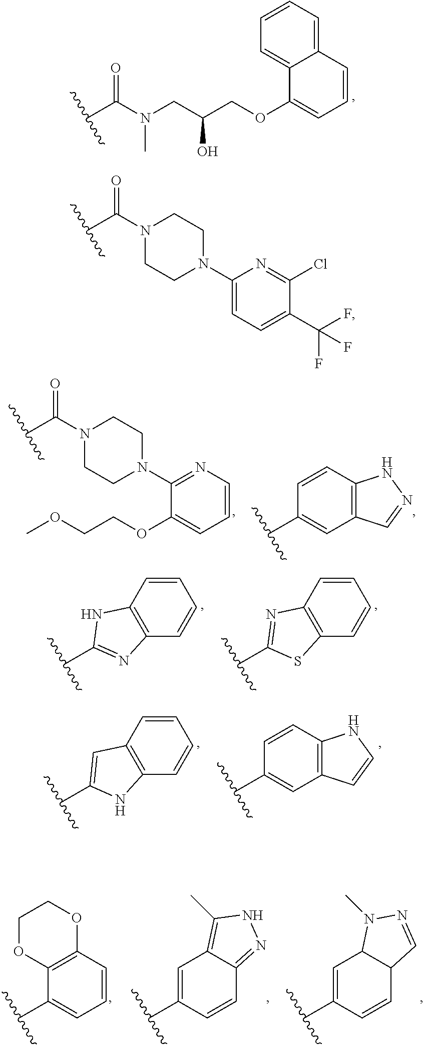

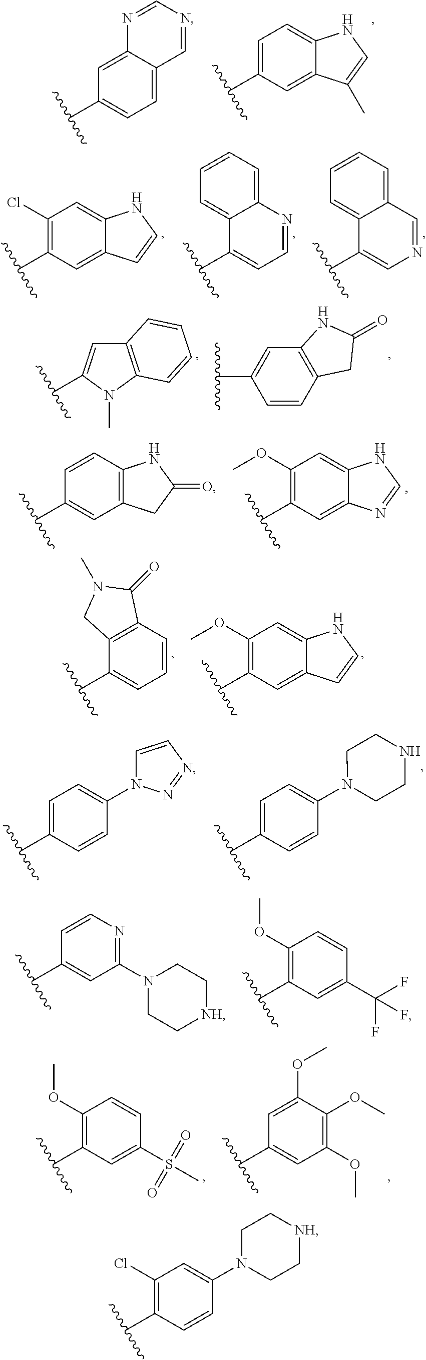

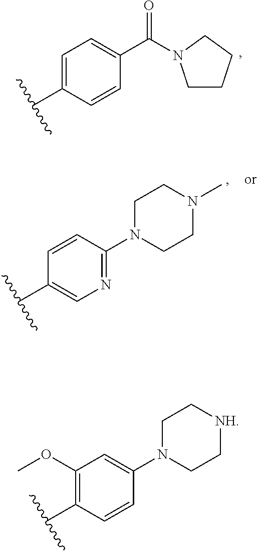

In some embodiments, R^{W} is

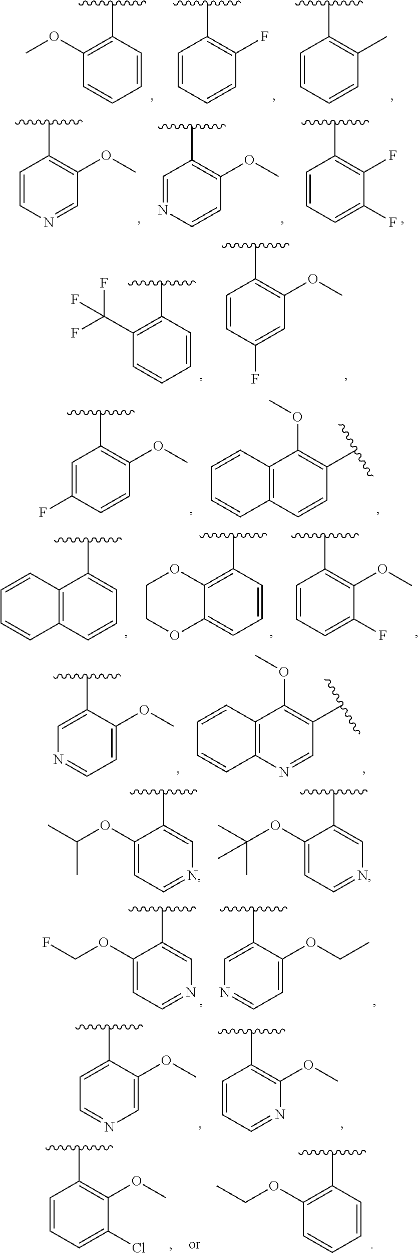

In some embodiments, R^{w} is —S(O)₂NH₂, S(O)₂N(CH₃)₂, —S(O)(NH)CH₃,

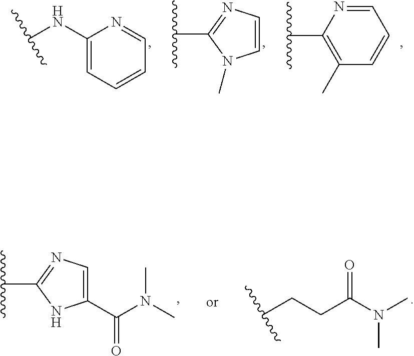

In some embodiments, R^{w} is

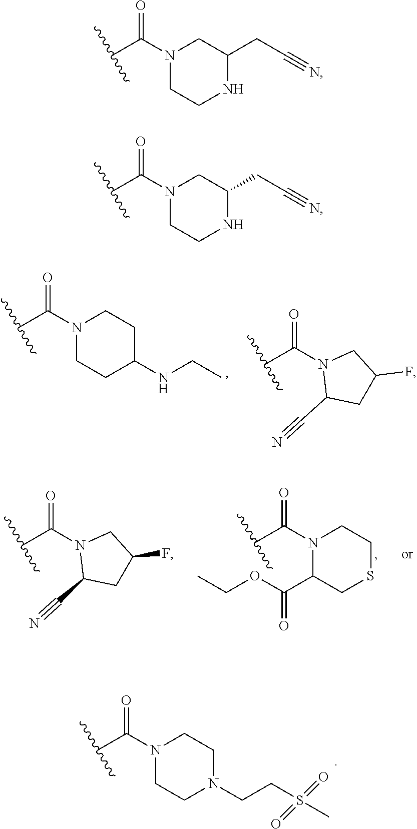

In some embodiments, R^{w} is

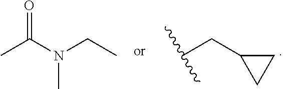

In some embodiments, R is selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)N(R)₂, and —NRS(O)₂R.

In some embodiments, R^{x} is hydrogen. In some embodiments, R^{x} is R^{A}. In some embodiments, R is halogen. In some embodiments, R is —CN. In some embodiments, R^{w} is —NO₂. In some embodiments, R^{w} is-OR. In some embodiments, R^{w} is-SR. In some embodiments, R is —NR₂. In some embodiments, R is —SiR₃. In some embodiments, R is —S(O)₂R. In some embodiments, R^{x} is —S(O)₂NR₂. In some embodiments, R^{X} is —S(O)(NR)R. In some embodiments, R^{x} is —S(O)R. In some embodiments, R^{w} is —C(O)R. In some embodiments, R^{w} is —C(O)OR. In some embodiments, R^{w} is —C(O)NR₂. In some embodiments, R^{X} is —C(O)NROR. In some embodiments, R^{X} is —OC(O)R. In some embodiments, R^{X} is —OC(O)NR₂. In some embodiments, R^{X} is —P(O)R₂. In some embodiments, R^{h} is —P(O)(OR)₂. In some embodiments, R is —OP(O)R₂. In some embodiments, R^{X} is —OP(O)(OR)₂. In some embodiments, R is —OP(O)(OR)NR₂. In some embodiments, R, is —OP(O)(NR₂)₂. In some embodiments, R^{x} is —NRC(O)OR. In some embodiments, R is —NRC(O)R. In some embodiments, R^{w} is —NRC(O)N(R)₂. In some embodiments, R is —NP(O)R₂. In some embodiments, R¹ is —NRP(O)(OR)₂. In some embodiments, R^{w} is —NRP(O)(OR)NR₂. In some embodiments, R^{w} is —NRP(O)(NR₂)₂. In some embodiments, R^{X} is —NRS(O)₂R.

In some embodiments, each R is independently selected from R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R

In some embodiments, RN is optionally substituted C₁₋₆ aliphatic. In some embodiments, RN is C₁₋₆ aliphatic optionally substituted with —OR^{o}, wherein R^{o} is hydrogen or C₁₋₆ aliphatic.

In some embodiments, RN is —CO₂Me, —CH₂OH, —C(O)Me, —C(O)Et, —C(O)iPr. —C(O)cyclopropyl, —C(O)oxetanyl, —C(O)tetrahydropyranyl, or pyridyl.

In some embodiments, RN is fluoro. In some embodiments, R^{x} is —S(O)₂CH₃. In some embodiments, RN is —C(O)N(CH₃)₂.

In some embodiments, R is —C(O)R or —C(O)OR. In some embodiments, R^{x} is —C(O)R.

In some embodiments, R is —C(O)R, wherein R of R^{w} is optionally substituted C₁₋₆ aliphatic. In some embodiments, R^{x} is —C(O)R, wherein R of R is C₁₋₆ aliphatic. In some embodiments, R is —C(O)R, wherein R of R is methyl, ethyl, n-propyl, or isopropyl. In some embodiments, R^{w} is —C(O)R, wherein R of R^{x} is C₁₋₆ aliphatic substituted with —OR^{o} or —C(O)OR^{o}, wherein R^{o} is hydrogen or C₁₋₆ aliphatic. In some embodiments, R is —C(O)R, wherein R of R is C₁₋₆ aliphatic substituted with —OR^{o}, wherein R^{o} is hydrogen or C₁₋₆ aliphatic. In some embodiments, R^{w} is —C(O)R, wherein R of R^{w} is C₁₋₆ aliphatic substituted with —C(O)OR^{o}, wherein R^{o} is hydrogen or C₁₋₆ aliphatic.

In some embodiments, R^{x} is

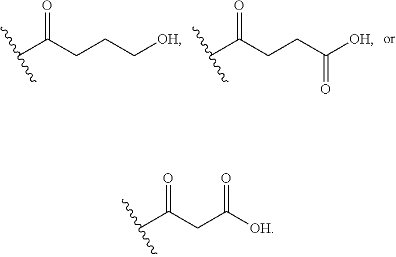 [SMILES: *C(=O)CCCO ; *C(=O)CCC(=O)O ; "or" ; *C(=O)CC(=O)O]

In some embodiments, R^{X} is —C(O)R, wherein R of R^{x} is C₁₋₆ aliphatic optionally substituted with —C(O)OR^{o}, —OR^{o}, halogen (e.g., fluoro), or ═O. In some embodiments, R^{x} is —C(O)OR. In some embodiments, R^{x} is —C(O)OR, wherein R of R^{X} is an optionally substituted C₁₋₆ aliphatic.

In some embodiments, R^{x} is

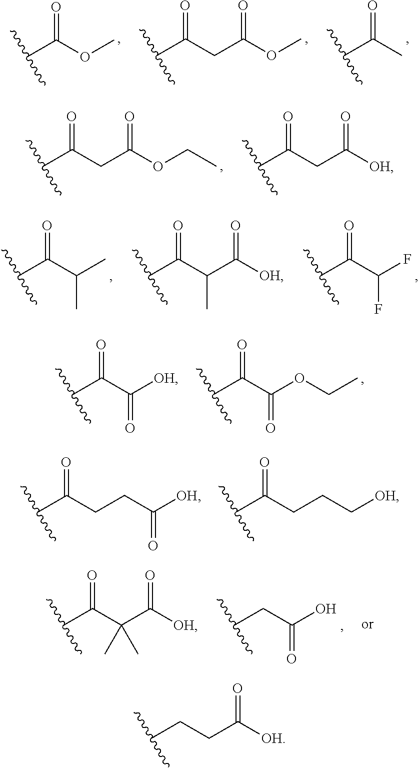

In some embodiments, each R^{y} is independently selected from R^{A}, halogen, —CN, —NO₂, OR—SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R is hydrogen. In some embodiments, R^{y} is R^{A}. In some embodiments, R^{Y} is halogen. In some embodiments, R^{Y} is —CN. In some embodiments, R^{Y} is —NO₂. In some embodiments, R^{Y} is —OR. In some embodiments, R^{y} is —SR. In some embodiments, R^{Y} is —NR₂. In some embodiments, R^{Y} is —SiR₃. In some embodiments, R^{Y} is —S(O)₂R. In some embodiments, R^{Y} is —S(O)₂NR₂. In some embodiments, R is —S(O)(NR)R. In some embodiments, R^{y} is —S(O)R. In some embodiments, R^{y} is —C(O)R. In some embodiments, R^{Y} is —C(O)OR. In some embodiments, RV is —C(O)NR₂. In some embodiments, R is —C(O)NROR. In some embodiments, R is —OC(O)R. In some embodiments, R is —OC(O)NR₂. In some embodiments, R^{Y} is —P(O)R₂. In some embodiments, R^{Y} is —P(O)(OR)₂. In some embodiments, R is —OP(O)R₂. In some embodiments, R^{Y} is —OP(O)(OR)₂. In some embodiments, R^{y} is —OP(O)(OR)NR₂. In some embodiments, R^{y} is —OP(O)(NR₂)₂. In some embodiments, R^{y} is —NRC(O)OR. In some embodiments, R^{y} is —NRC(O)R. In some embodiments, R^{y} is —NRC(O)N(R)₂. In some embodiments, RV is —NP(O)R₂. In some embodiments, RV is —NRP(O)(OR)₂. In some embodiments, R^{Y} is —NRP(O)(OR)NR₂. In some embodiments, R^{Y} is —NRP(O)(NR₂)₂. In some embodiments, R is —NRS(O)₂R.

In some embodiments, R is fluoro, chloro, bromo, iodo, methyl, ethyl, cyclopropyl, —CF₃, —CN, CH₂O, —CO₂H, —CO₂Me, —CO₂tBu, —C(O)Me, —NH₂, —NHMe, —NHAc, —NHC(O)Et, —OH, —OMe, —OCH₂CH₂NH₂, —CH₂OH, —CH₂OMe, —CH₂NHMe, —CH₂NHAc, —CH₂SO₂Me, —SO₂Me, —SO₂NH₂, -SO₂NHMe,

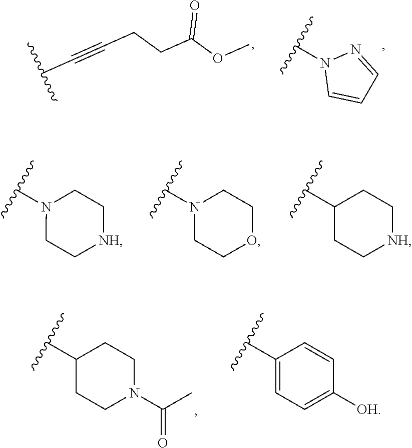

In some embodiments, R^{y} is —C(O)H.

In some embodiment, R^{w}, R^{x}, and R^{y} are as depicted in the compounds of Table 1B, below.

As described above and defined herein, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic heteroaryl ring having 1-4 heteroatoms independently selected from nitrogen, oxygen, and sulfur.

In some embodiments, each R^{A} is independently selected from C₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, a 3-7 membered saturated heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic saturated heterocyclic ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, a 5-10 membered monocyclic or bicyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or a monocyclic heteroaryl ring having 1-4 heteroatoms independently selected from nitrogen, oxygen, and sulfur.

In some embodiments, each R^{A} is independently an optionally substituted group selected from C₁₋₆ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments, R^{A} is an optionally substituted R^{A} is a 5-10 membered monocyclic or bicyclic heteroaryl ring having 1-4 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is optionally substituted naphthalenyl. In some embodiments, R^{A} is optionally substituted dihydrobenzo[b][1A]dioxinyl.

In some embodiments, R^{A} is an optionally substituted 8-11 membered monocyclic or bicyclic aryl or heteroaryl with 1-4 heteroatoms independently selected from nitrogen, oxygen and sulfur. In some embodiments, R^{A} is optionally substituted naphthalenyl or dihydrobenzo[b][1,4]dioxinyl. In some embodiments, R^{A} is optionally substituted quinolinyl.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1B, below.

As described above and defined herein, each R^{B} is independently -L^{B}-Cy^{B1}-H or -L^{B}-Cy^{B1}-Cy^{B2}.

In some embodiments, R^{B} is -L^{B}-Cy^{B1}-H. In some embodiments, R^{B} is -L^{B}-Cy^{B1}-Cy^{B2}.

In some embodiments, R^{B} is

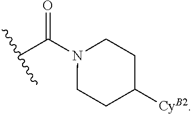

In some embodiments, R^{B} is

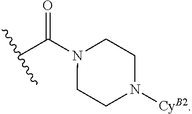

In some embodiments, R^{B} is

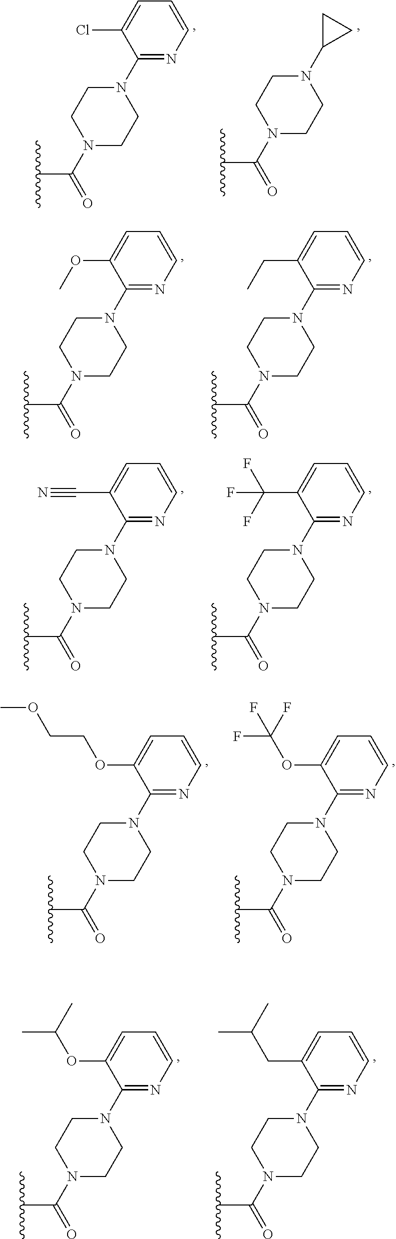

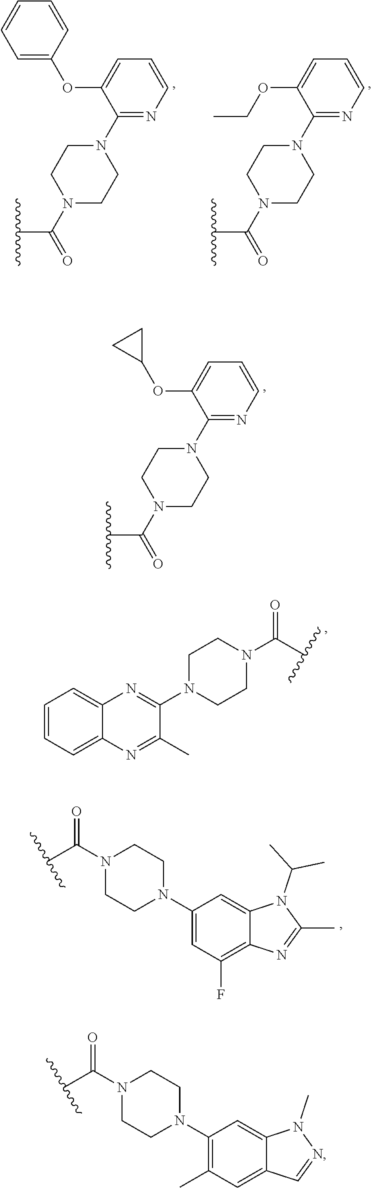 [SMILES: *C(=O)N1CCN(c2ncccc2Oc2ccccc2)CC1 ; *C(=O)N1CCN(c2ncccc2OCC)CC1 ; *C(=O)N1CCN(c2ncccc2OC2CC2)CC1 ; *C(=O)N1CCN(c2nc3ccccc3nc2C)CC1 ; *C(=O)N1CCN(c2cc(F)c3nc(C)n(C(C)C)c3c2)CC1 ; *C(=O)N1CCN(c2cc3c(cnn3C)cc2C)CC1]

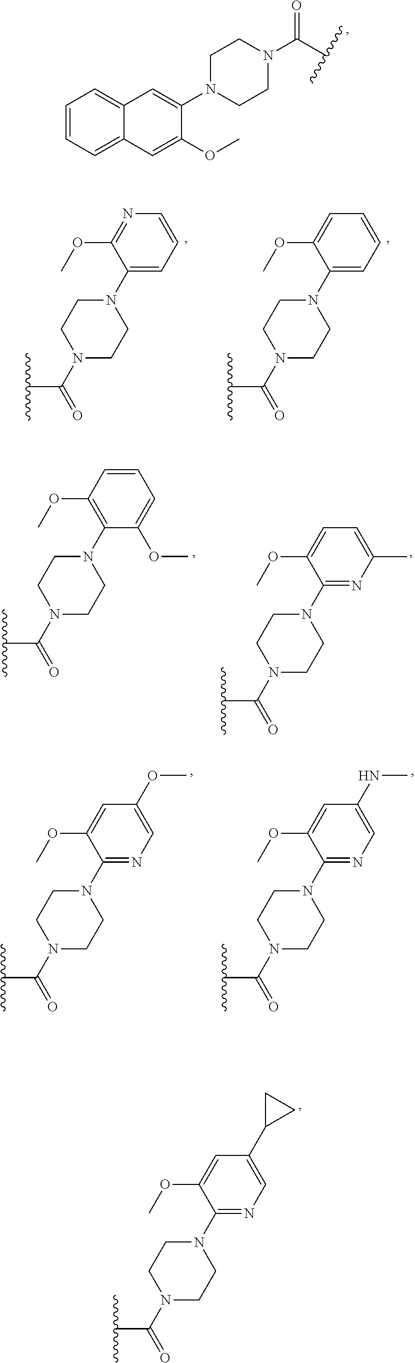

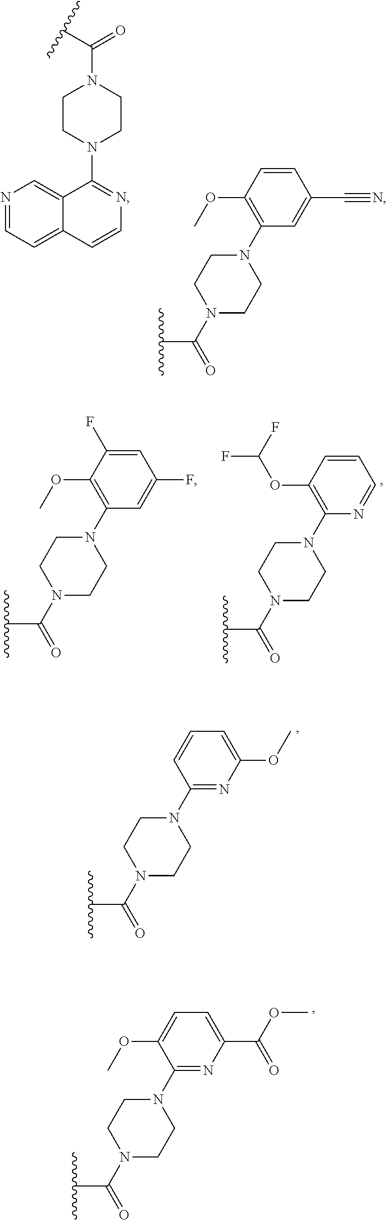

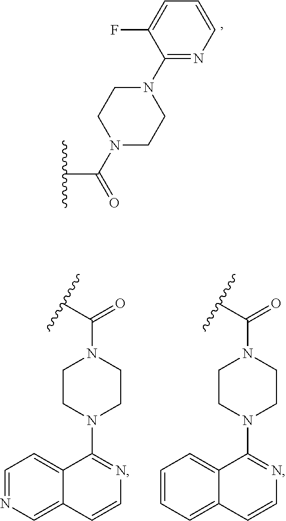

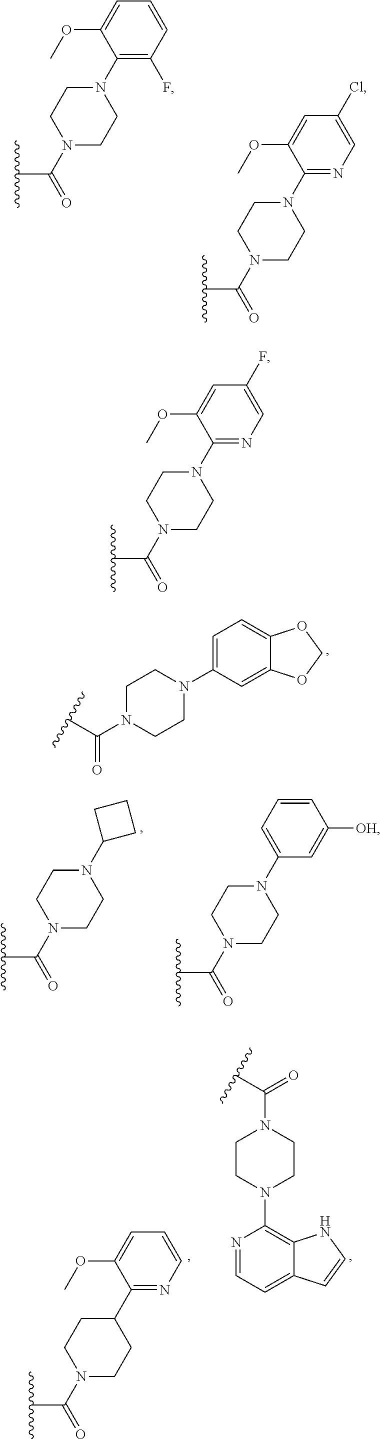

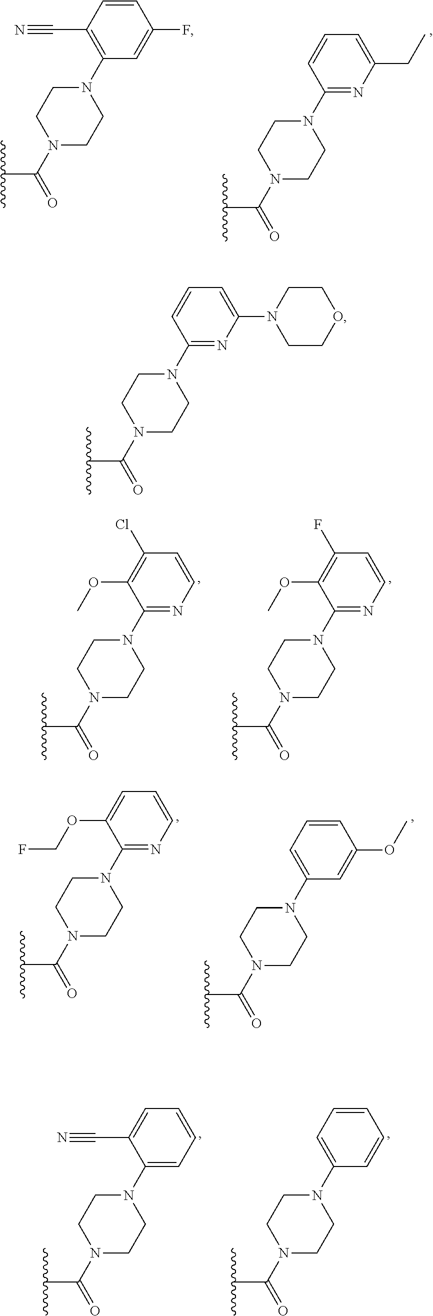

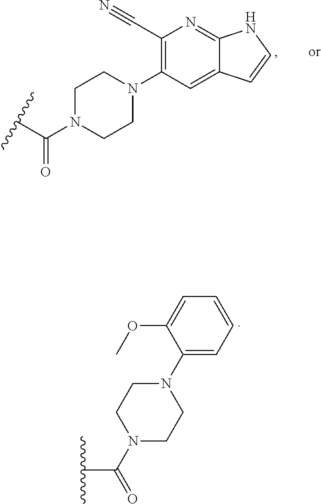 [SMILES: *C(=O)N1CCN(c2cc3cc[nH]c3nc2C#N)CC1 ; *C(=O)N1CCN(c2ccccc2OC)CC1]

As described above and defined herein, each L^{B} is independently a covalent bond or a C₁-3 bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—

In some embodiments, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—.

In some embodiments, L^{B} is a covalent bond. In some embodiments, L^{B} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—. In some embodiments, L^{B} is a C₁₋₃ bivalent straight or branched saturated or unsaturated hydrocarbon chain wherein 1-2 methylene units of the chain are independently and optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1-2 methylene units of the chain are independently and optionally replaced with —O—, —C(O)—, or —NR—. In some embodiments, L^{B} is a C₁₋₃ bivalent straight or branched saturated or unsaturated hydrocarbon chain wherein 1 methylene unit of the chain is optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1 methylene unit of the chain is optionally replaced with —O—, —C(O)—, or —NR—. In some embodiments, L^{B} is a C₁₋₃ bivalent straight or branched saturated or unsaturated hydrocarbon chain wherein 1 methylene unit of the chain is replaced with —C(O)—. In some embodiments, L^{B} is —C(O)—.

In some embodiments, L^{B} is a C₁₋₃ bivalent straight or branched saturated or unsaturated hydrocarbon chain. In some embodiments, L^{B} is —CH₂—.

In some embodiments, L^{B} is —C(O)—, —C(S)—, —C(NR)R—, —S(O)—, —S(O)₂— or —S(O)(NR)—. In some embodiments, L^{B} is —C(O)—, —C(S)—, or —C(NR)R—. In some embodiments, L^{B} is —C(O)—, —S(O)—, —S(O)₂— or —S(O)(NR)—. In some embodiments, L^{B} is —C(S)—. In some embodiments, L^{B} is —C(NR)R—. In some embodiments, L^{B} is —S(O)—. In some embodiments, L^{B} is —S(O)₂—. In some embodiments, L^{B} is —S(O)(NR)—.

As described above and defined herein, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

It will be appreciated that all embodiments to Cy^{B1} may refer to a terminal ring (or otherwise optionally substituted ring) in structures with -L^{B}-Cy^{B1}, or a ring further connected to Cy^{B2} as in structures -L^{B}-Cy^{B1}-Cy^{B2}, regardless of how presented. By way of example, an embodiment to Cy^{B1} is phenylenyl, refers to phenylenyl in -L^{B}-Cy^{B1}-Cy^{B2}, and phenyl in -L^{B}-Cy^{B1}. Similarly, an embodiment to Cy^{B1} is

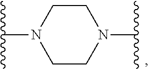 [SMILES: *N1CCN(*)CC1]

refers to

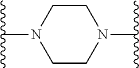 [SMILES: *N1CCN(*)CC1]

in -L^{B}-Cy^{B1}-Cy^{B2}, and

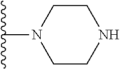 [SMILES: *N1CCNCC1]

in -L^{B}-Cy^{B1}. Similarly, an embodiment to Cy^{B1} is

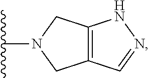 [SMILES: *N1Cc2cn[nH]c2C1]

refers to

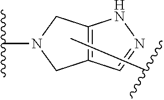

in -L^{B}-Cy^{B1}-Cy^{B2}, and

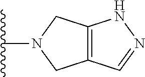 [SMILES: *N1Cc2cn[nH]c2C1]

In some embodiments, each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

In some embodiments, Cy^{B1} is optionally substituted phenylenyl. In some embodiments, Cy^{B1} is phenylenyl.

In some embodiments, Cy^{B1} is optionally substituted 3-7 membered saturated or partially unsaturated carbocyclylenyl or heterocyclylenyl having 1-4 heteroatoms independently selected from nitrogen, oxygen, and sulfur. In some embodiments, Cy^{B1} is optionally substituted 5-10 membered monocyclic or bicyclic arylenyl or heteroarylenyl having 1-4 heteroatoms independently selected from nitrogen, oxygen, and sulfur.

In some embodiments, Cy^{B1} is optionally substituted 3-7 membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-2 heteroatoms independently selected from nitrogen, oxygen, and sulfur. In some embodiments, Cy^{B1} is optionally substituted 6-membered saturated or partially unsaturated heterocyclylenyl having 1-2 nitrogen heteroatoms. In some embodiments, Cy^{B1} is optionally substituted piperadinylenyl or piperazinylenyl.

In some embodiments, Cy^{B1} is a 3-7 membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-2 heteroatoms independently selected from nitrogen, oxygen, and sulfur. In some embodiments, Cy^{B1} is a 6-membered saturated or partially unsaturated heterocyclylenyl having 1-2 nitrogen heteroatoms. In some embodiments, Cy^{B1} is a piperadinylenyl or piperazinylenyl. In some embodiments, Cy^{B1} is

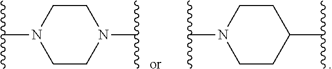 [SMILES: *N1CCN(*)CC1 ; *C1CCN(*)CC1 ; "or" ; "."]

In some embodiments, Cy^{B1} is an optionally substituted piperidinonyl or piperazinonyl. In some embodiments, Cy^{B1} is an optionally substituted dihydropyridinyl. In some embodiments, Cy^{B1} is an optionally substituted thiomorpholinyl.

In some embodiments, Cy^{B1} is a 3-10 membered saturated or partially unsaturated monocyclic or bicyclic heterocyclylenyl having 1-4 heteroatoms independently selected from nitrogen, oxygen, and sulfur.

In some embodiments, Cy^{B1} is an optionally substituted 8-10 membered saturated or partially unsaturated bicyclic heterocyclylenyl having 1-4 heteroatoms independently selected from nitrogen, oxygen, and sulfur. In some embodiments, Cy^{B} is an optionally substituted 8-membered saturated or partially unsaturated heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-2 heteroatoms independently selected from nitrogen, oxygen, and sulfur. In some embodiments, Cy^{B1} is an optionally substituted 8-membered saturated or partially unsaturated heterocyclylenyl having 1-2 nitrogen heteroatoms. In some embodiments, Cy^{B1} is optionally substituted

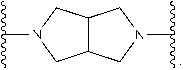 [SMILES: *N1CC2CN(*)CC2C1]

In some embodiments, Cy^{B1} is an optionally substituted

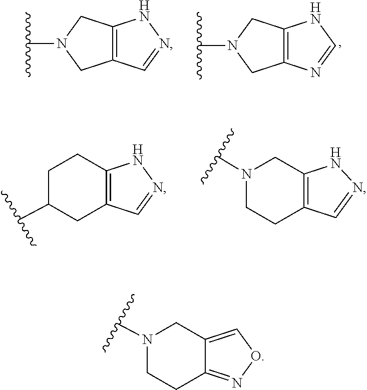

In some embodiments, Cy^{B1} is an optionally substituted 5-6 membered monocyclic heteroarylenyl having 1-4 heteroatoms independently selected from nitrogen, oxygen, and sulfur. In some embodiments, Cy^{B1} is an optionally substituted 6 membered monocyclic heteroarylenyl having 1-4 heteroatoms independently selected from nitrogen, oxygen, and sulfur. In some embodiments, Cy^{B1} is an optionally substituted pyridinonyl, pyrazinonyl, or pyrimidinonyl.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, Cy^{B2} is optionally substituted phenyl. In some embodiments, Cy^{B2} is phenyl. In some embodiments, Cy^{B2} is phenyl, optionally substituted with —CN, halogen, —R^{o}, or —OR^{o}, wherein R^{o} is hydrogen or C₁₋₆ aliphatic.

In some embodiments, Cy^{B2} is optionally substituted 3-7 membered saturated or partially unsaturated carbocyclic or heterocyclic ring having 1-2 heteroatoms independently selected from nitrogen, oxygen, and sulfur.

In some embodiments, Cy^{B2} is optionally substituted 3-7 membered saturated or partially unsaturated carbocyclic. In some embodiments, Cy^{B2} is optionally substituted cyclopropyl. In some embodiments, Cy^{B2} is cyclopropyl. In some embodiments, Cy^{B2} is optionally substituted cyclobutyl. In some embodiments, Cy^{B2} is cyclobutyl.

In some embodiments, Cy^{B2} is optionally substituted cyclopentyl. In some embodiments, Cy^{B2} is cyclopentyl. In some embodiments, Cy^{B2} is optionally substituted cyclohexyl. In some embodiments, Cy^{B2} is cyclohexyl.

In some embodiments, Cy^{B2} is optionally substituted 5-10 membered monocyclic or bicyclic aryl or heteroaryl with 1-4 heteroatoms independently selected from nitrogen, oxygen and sulfur.

In some embodiments, Cy^{B2} is optionally substituted 5-6 membered monocyclic heteroaryl with 1-4 heteroatoms independently selected from nitrogen, oxygen and sulfur. In some embodiments, Cy^{B2} is optionally substituted 5-6 membered monocyclic heteroaryl with 1-2 heteroatoms independently selected from nitrogen, oxygen and sulfur. In some embodiments, Cy^{B2} is optionally substituted pyridinyl. In some embodiments, Cy^{B2} is pyridinyl, optionally substituted with —CN, halogen, —R^{o}, —OR^{o}, —N(R^{o})₂—C(O)OR^{o}, wherein each R^{o} is independently hydrogen; C₁₋₆ aliphatic, which may be optionally substituted with halogen, —(CH₂)₀₋₂OH, or —(CH₂)₀₋₂OR^{•}, where R^{•} is C₁₋₄ aliphatic; or a 5-6-membered saturated, partially unsaturated, or aryl ring having 0-4 heteroatoms independently selected from nitrogen, oxygen, or sulfur (e.g., phenyl or morpholinyl).

In some embodiments, Cy^{B2} is optionally substituted 6 membered monocyclic heteroaryl with 1-2 heteroatoms independently selected from nitrogen, oxygen and sulfur. In some embodiments, Cy^{B2} is optionally substituted pyridinyl, pyrimidinyl, pyridazinyl, pyrazinyl, or triazinyl.

In some embodiments, Cy^{B2} is optionally substituted optionally substituted pyridinonyl, pyrazinonyl, or pyrimidinonyl

In some embodiments, Cy^{B2} is optionally substituted 5 membered monocyclic heteroaryl with 1-2 heteroatoms independently selected from nitrogen, oxygen and sulfur. In some embodiments, Cy^{B2} is optionally substituted pyrazolyl, imidazolyl, or triazolyl.

In some embodiments, Cy^{B2} is optionally substituted 8-10 membered bicyclic aryl or heteroaryl with 1-4 heteroatoms independently selected from nitrogen, oxygen and sulfur.

In some embodiments, Cy^{B2} is optionally substituted 9-membered bicyclic heteroaryl with 1-4 heteroatoms independently selected from nitrogen, oxygen and sulfur. In some embodiments, Cy^{B2} is optionally substituted 9-membered bicyclic heteroaryl with 1-4 nitrogen heteroatoms. In some embodiments, Cy^{B2} is optionally substituted benzimidazolyl, indazolyl, or azaindolyl (e.g., pyrrolo[2,3-c]pyridinyl or pyrrolo]2,3-b]pyridinyl). In some embodiments, Cy^{B2} is benzimidazolyl indazolyl, or azaindolyl (e.g., pyrrolo[2,3-c]pyridinyl or pyrrolo]2,3-b]pyridinyl) optionally substituted with —CN, halogen or —R^{o}, wherein R^{o} is hydrogen or C₁₋₆ aliphatic. In some embodiments, Cy^{B2} is optionally substituted indolyl or azaindolyl.

In some embodiments, Cy^{B2} is optionally substituted 10-membered bicyclic aryl or heteroaryl with 1-4 heteroatoms independently selected from nitrogen, oxygen and sulfur. In some embodiments, Cy^{B2} is optionally substituted 10-membered bicyclic heteroaryl with 1-4 nitrogen heteroatoms. In some embodiments, Cy^{B2} is optionally substituted quinoxalinyl, isoquinolinyl, 2,6-naphthyridinyl, or 2,7-naphthyridinyl. In some embodiments, Cy^{B2} is quinoxalinyl, isoquinolinyl, 2,6-naphthyridinyl, or 2,7-naphthyridinyl, optionally substituted with —R^{o}, wherein R^{o} is hydrogen or C₁₋₆ aliphatic.

In some embodiments, Cy^{B2} is optionally substituted naphthalenyl. In some embodiments, Cy^{B2} is naphthalenyl, optionally substituted with —R^{o} or —OR^{o}, wherein R^{o} is hydrogen or C₁₋₆ aliphatic.

In some embodiments, Cy^{B2} is optionally substituted benzo[d][1,3]dioxolyl.

In some embodiments, Cy^{B2} is

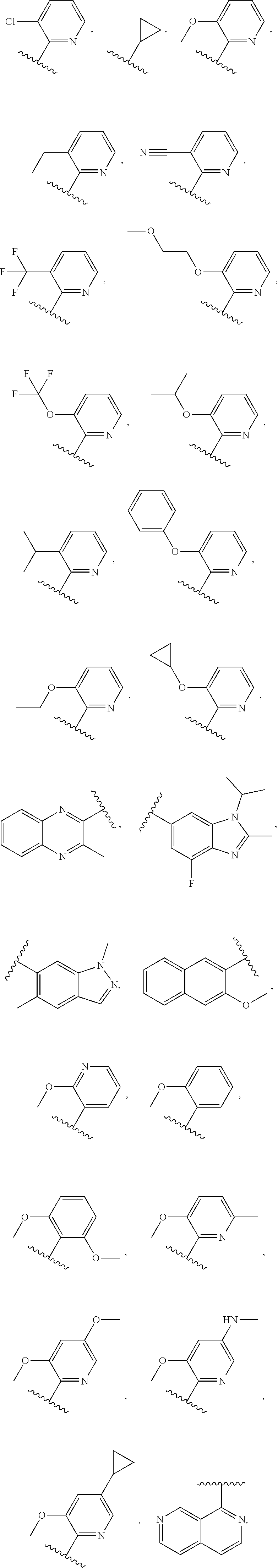

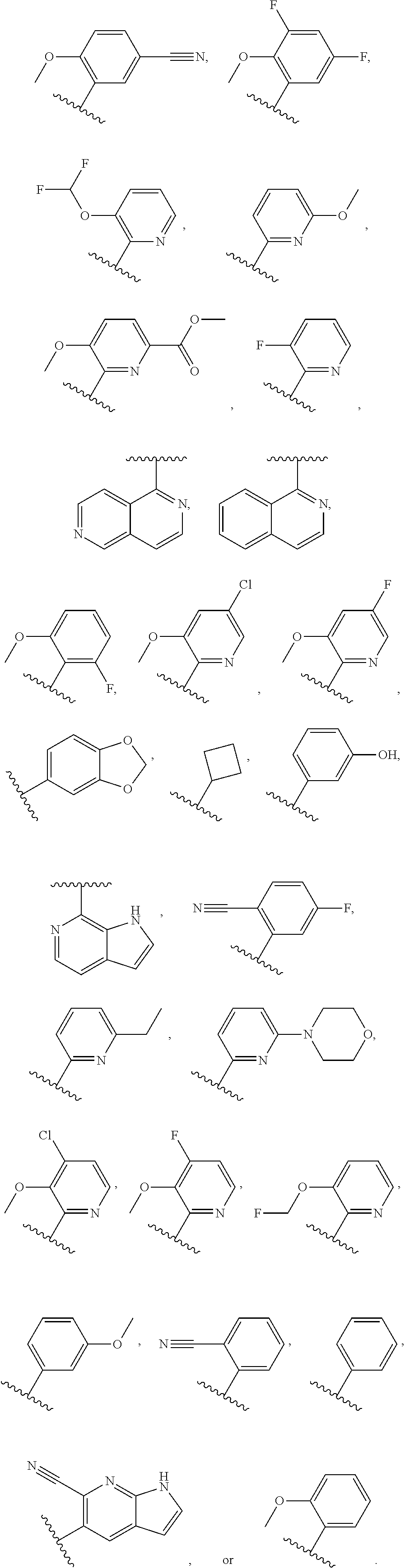

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur. In some embodiments, two R groups on adjacent atoms are optionally taken together with their intervening atoms to form a 3-7 membered saturated or partially unsaturated ring having 0-3 heteroatoms, in addition to the adjacent atoms to which they are attached, independently selected from nitrogen, oxygen, and sulfur.

In some embodiment, R is as depicted in the compounds of Table 1B, below.

As described above and defined herein. L^{X} is a covalent bond or an optionally substituted C₁₋₅ bivalent straight or branched saturated or unsaturated hydrocarbon chain wherein 1-2 methylene units of the chain are independently and optionally replaced with -Cy^{x}-, —O—, —C(O)—, —C(S)—, —CR₂—, —CF₂—, —CRF—, —CR(OR)—, —NR—, —C(NR)—, —S—, —S(O)—, —S(O)₂—, —S(O)(NR)—, or —CR═CR—.

In some embodiments, L^{X} is a covalent bond or an optionally substituted C₁₋₅ bivalent straight or branched saturated or unsaturated hydrocarbon chain wherein 1-2 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{X} is a covalent bond or an optionally substituted C₁₋₅ bivalent straight or branched saturated or unsaturated hydrocarbon chain wherein 1-2 methylene units of the chain are independently and optionally replaced with -Cy^{X}-, —O—, —C(O)—, —C(S)—, —CR₂—, —CF₂—, —CRF—, —CR(OR)—, —NR—, —S—, —S(O)—, —S(O)₂— —S(O)(NR)—, or —CR═CR—.

In some embodiments, L^{x} is an optionally substituted C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{X} is an optionally substituted C₁₋₄ bivalent straight or branched saturated or unsaturated hydrocarbon chain wherein 1-3 methylene units of the chain are independently and optionally replaced with -Cy^{X}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{X} is an optionally substituted C₁₋₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, L^{X} is an optionally substituted C₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—.

In some embodiments, L^{X} is a covalent bond. In some embodiments, LU is a C₁₋₅ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, LU is a C₁₋₄ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, LU is a C₁₋₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—. In some embodiments, LU is a C₃ bivalent straight or branched saturated or unsaturated hydrocarbon chain wherein 1-3 methylene units of the chain are independently and optionally replaced with -Cy^{x}-, —O—, —C(O)—, —C(S)—, —CR₂—, —CF₂—, —CRF—, —CR(OR)—, —NR—, —S—, —S(O)—, —S(O)₂— or —CR═CR—.

In some embodiments, L^{x} is an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{X} is an optionally substituted C₁₋₅ bivalent straight or branched saturated or unsaturated hydrocarbon chain wherein 1-2 methylene units of the chain are independently and optionally replaced with —O—, —C(O)—, or —NR—.

In some embodiments, L^{X} is an optionally substituted C₁₋₅ bivalent straight or branched saturated or unsaturated hydrocarbon chain wherein 1-2 methylene units of the chain are independently and optionally replaced with —C(O)—, —C(S)—, —CR₂—, —CF2-, —CRF—, —CR(OR)—, —S—, —S(O)—, or —S(O)₂. In some embodiments, L^{X} is a C₁₋₅ bivalent straight or branched saturated or unsaturated hydrocarbon chain wherein 1-2 methylene units of the chain are independently and optionally replaced with —C(O)—, —C(S)—, —CR₂—, —CF2-, —CRF—, —CR(OR)—, —S—, —S(O)—, or —S(O)₂, and wherein L^{X} is optionally substituted with halogen or —R^{o}.

In some embodiments, L^{X} is an optionally substituted C₁₋₅ bivalent straight or branched saturated or unsaturated hydrocarbon chain wherein one methylene unit is replaced with —C(O)—. In some embodiments, L^{X} is an optionally substituted C₁₋₄ bivalent straight or branched saturated or unsaturated hydrocarbon chain wherein one methylene unit is replaced with —C(O)—. In some embodiments, L^{X} is an optionally substituted C₁₋₃ bivalent straight or branched saturated or unsaturated hydrocarbon chain wherein one methylene unit is replaced with —C(O)—. In some embodiments, L^{x} is an optionally substituted C₃ bivalent straight or branched saturated or unsaturated hydrocarbon chain wherein one methylene unit is replaced with —C(O)—. In some embodiments, L^{x} is an optionally substituted C₃ bivalent straight saturated or unsaturated hydrocarbon chain wherein one methylene unit is replaced with —C(O)—.

In some embodiments, L^{x} is a C₁₋₅ bivalent straight or branched saturated or unsaturated hydrocarbon chain wherein one methylene unit is replaced with —C(O)—, and wherein L^{x} is optionally substituted with halogen or —R^{o}. In some embodiments, L^{x} is a C₁₋₄ bivalent straight or branched saturated or unsaturated hydrocarbon chain wherein one methylene unit is replaced with —C(O)—, and wherein L^{x} is optionally substituted with halogen or —R^{o}. In some embodiments, L^{x} is a Cis bivalent straight or branched saturated or unsaturated hydrocarbon chain wherein one methylene unit is replaced with —C(O)—, and wherein L^{x} is optionally substituted with halogen or —R^{o}. In some embodiments, L^{x} is a C₃ bivalent straight or branched saturated or unsaturated hydrocarbon chain wherein one methylene unit is replaced with —C(O)- and wherein L^{x} is optionally substituted with halogen or —R^{o}. In some embodiments, L^{x} is a C₃ bivalent straight saturated or unsaturated hydrocarbon chain wherein one methylene unit is replaced with —C(O)—, and wherein L^{x} is optionally substituted with halogen or —R^{o}.

In some embodiments, L¹ is —C(F)—CH-(optionally substituted C₁₋₃ bivalent straight or branched saturated or unsaturated hydrocarbon chain)-. In some embodiments, L¹ is —C(CF₃)—N(Rt)-(optionally substituted C₁₋₃ bivalent straight or branched saturated or unsaturated hydrocarbon chain)-

In some embodiments, L^{x} is a covalent bond or #-L^{XA}-L^{XB}-, wherein:
# represents the point of attachment to Ring X;
L^{XA} is -Cy^{x}-, —C(O)—, —C(S)—, —CR₂—, —CR(OR)—, —C(NR)—, —S(O)—, —S(O)₂— —S(O)(NR)—; and
L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

As described above and defined herein, L^{XA} is -Cy^{x}-, —C(O)—, —C(S)—, —CR₂—, —CR(OR)—, —C(NR)—, —S(O)—, —S(O)₂— or —S(O)(NR)—. In some embodiments, L^{XA} is -Cy^{x}-. In some embodiments, L^{XA} is —C(O)—, —C(S)—, —CR₂—, —CR(OR)—, —C(NR)—, —S(O)—, —S(O)₂— or —S(O)(NR)—.

In some embodiments, L^{XA} is —C(O)— or —C(S)—. In some embodiments, L^{XA} is —C(O)—, —S(O)—, —S(O)₂— or —S(O)(NR)—. In some embodiments, L^{XA} is —S(O)—, —S(O)₂— or —S(O)(NR)—. In some embodiments, L^{XA} is —C(O)—. In some embodiments, L^{XA} is —C(S)—. In some embodiments, L^{XA} is —CR₂—. In some embodiments, L^{XA} is —CR₂—, wherein each R of L^{XA} is independently hydrogen or optionally substituted C₁₋₆ aliphatic. In some embodiments, L^{XA} is —CR₂—, wherein each R of L^{XA} is independently hydrogen or C₁₋₆ aliphatic substituted with halogen (e.g., fluoro). In some embodiments, L^{XA} is —CR(OR)—. In some embodiments, L^{XA} is —C(NR)—. In some embodiments, L^{XA} is —S(O)—. In some embodiments, L^{XA} is —S(O)₂—. In some embodiments, L^{XA} is —S(O)(NR)—.

In some embodiments, L^{XA} is an optionally substituted phenyl. In some embodiments, L^{A} is an optionally substituted 3-7 membered saturated or partially unsaturated carbocyclylenyl. In some embodiments, L^{XA} is an optionally substituted 3-7 membered saturated or partially unsaturated heterocyclylenyl having 1-4 heteroatoms independently selected from nitrogen, oxygen, and sulfur. In some embodiments, L^{XA} is an optionally substituted 5-9 membered monocyclic or bicyclic heteroarylenyl having 1-4 heteroatoms independently selected from nitrogen, oxygen, and sulfur, some embodiments, L^{XA} is an optionally substituted 5-6 membered monocyclic heteroarylenyl having 1-4 heteroatoms independently selected from nitrogen, oxygen, and sulfur. In some embodiments, L^{XA} is an optionally substituted 5 membered monocyclic heteroarylenyl having 1-4 heteroatoms independently selected from nitrogen, oxygen, and sulfur. In some embodiments, L^{XA} is an optionally substituted 6 membered monocyclic heteroarylenyl having 1-4 heteroatoms independently selected from nitrogen, oxygen, and sulfur.

In some embodiments, L^{XA} is an optionally substituted

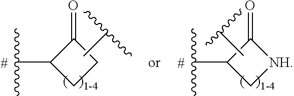

In some embodiments, L^{XA} is an optionally substituted

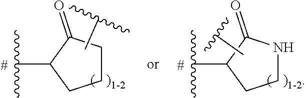

In some embodiments, L^{XA} is an optionally substituted:

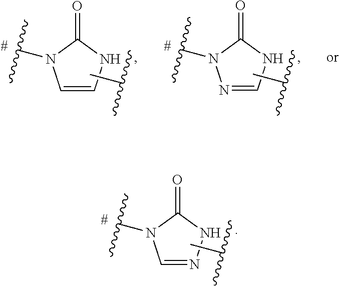

In some embodiments, L^{XA} is:

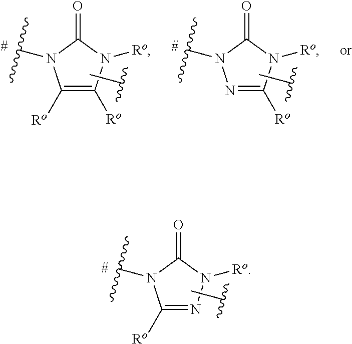

In some such embodiments, R^{o} is hydrogen or C₁₋₆ aliphatic.

In some embodiments, L^{XA} is an optionally substituted:

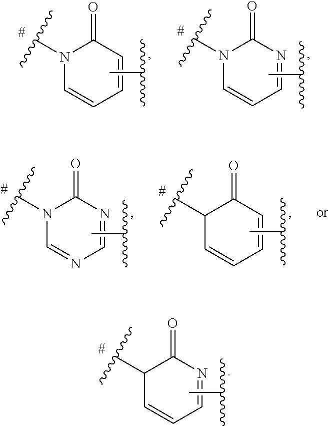

In some embodiments, L¹ is:

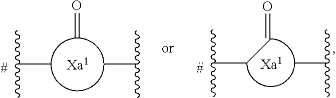

wherein Ring Xa¹ is an optionally substituted 3-7 membered saturated or partially unsaturated carbocyclic or heterocyclic ring having 1-2 heteroatoms selected from nitrogen, oxygen, and sulfur, or a 5-6 membered monocyclic heteroaryl ring having 1-4 heteroatoms independently selected from nitrogen, oxygen, and sulfur.

As defined above and described herein, Ring Xa¹ is an optionally substituted 3-7 membered saturated or partially unsaturated carbocyclyl or heterocyclyl having 1-2 heteroatoms selected from nitrogen, oxygen, and sulfur, or a 5-6 membered monocyclic heteroaryl having 1-4 heteroatoms independently selected from nitrogen, oxygen, and sulfur. In some embodiments, Ring Xa¹ is an optionally substituted 3-7 membered saturated or partially unsaturated carbocyclyl. In some embodiments, Ring Xa¹ is an optionally substituted 3-7 membered saturated or partially unsaturated heterocyclyl having 1-2 heteroatoms selected from nitrogen, oxygen, and sulfur, 5-6 membered monocyclic heteroaryl having 1-4 heteroatoms independently selected from nitrogen, oxygen, and sulfur.

In some embodiments, Ring Xa¹ an optionally substituted 5 membered monocyclic heteroaryl ring having 1-4 heteroatoms independently selected from nitrogen, oxygen, and sulfur. In some embodiments, Ring Xa¹ is an optionally substituted 6 membered monocyclic heteroaryl ring having 1-4 heteroatoms independently selected from nitrogen, oxygen, and sulfur.

In some embodiments, L^{XA} is

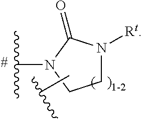

In some embodiments, L^{XA} is optionally substituted

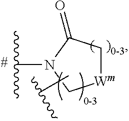

where W^{m} is as described above and defined herein. In some embodiments, L^{XA} is

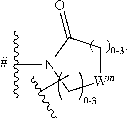

In some embodiments, L^{XA} is optionally substituted

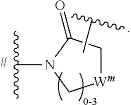

In some embodiments, L^{XA} is

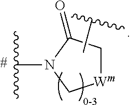

In some embodiments, L^{XA} is

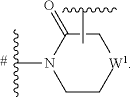

In some embodiments, L^{XA} is

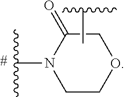

In some embodiments, L^{XA} is an optionally substituted ring selected from:

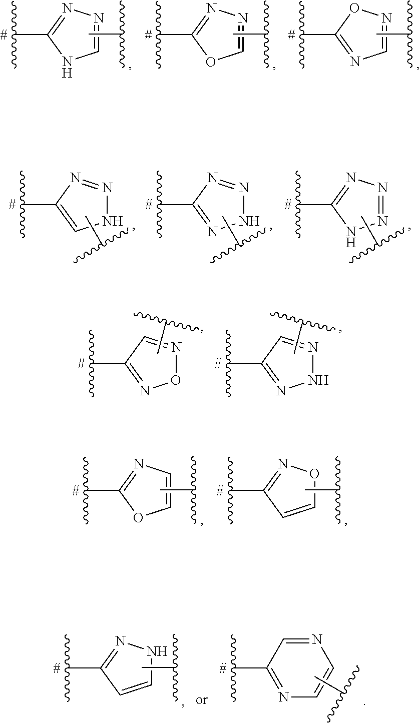

In some embodiments, L¹ is:

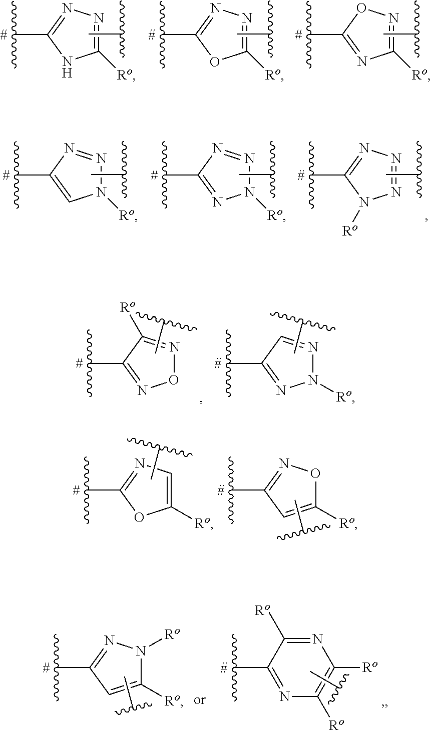

wherein each R^{o} is a defined above and described herein (e.g., hydrogen or C₁₋₆ aliphatic.

In some embodiments, L^{X} is:

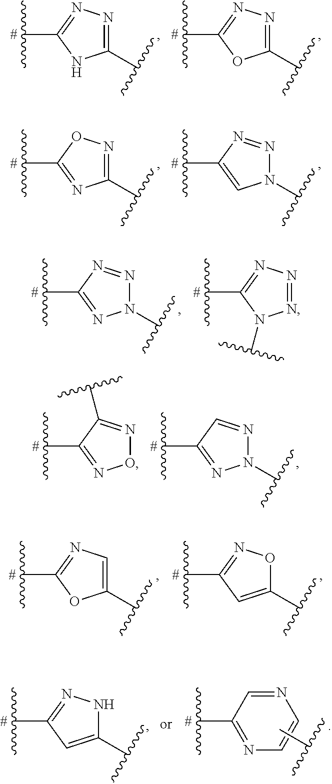

As described above and defined herein, L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L¹ is a covalent bond. In some embodiments, L^{XB} is an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L¹ is an optionally substituted C₁₋₄ bivalent straight or branched saturated or unsaturated hydrocarbon chain. In some embodiments, L¹ is an optionally substituted C₁₋₄ bivalent straight or branched saturated or unsaturated hydrocarbon chain, wherein 1 methylene unit of the chain is optionally replaced with —O—, —NR—, or —S—.

In some embodiments, L¹ is an optionally substituted C₁₋₃ bivalent straight or branched saturated or unsaturated hydrocarbon chain. In some embodiments, L¹ is an optionally substituted C₁a bivalent straight or branched saturated or unsaturated hydrocarbon chain, wherein 1 methylene unit of the chain is optionally replaced with —O—, —NR—, or —S—. In some embodiments, L¹ is —NR-(optionally substituted C₁₋₄ bivalent straight or branched saturated or unsaturated hydrocarbon chain)-. In some embodiments, L¹ is —O-(optionally substituted C₁₋₄ bivalent straight or branched saturated or unsaturated hydrocarbon chain)-. In some embodiments, L¹ is —S-(optionally substituted C₁₋₄ bivalent straight or branched saturated or unsaturated hydrocarbon chain)-.

In some embodiments, L¹ is optionally substituted —CH₂—, —CH₂CH₂—, —CH₂CH₂CH₂—, —CH₂CH₂CH₂O—, —CH₂C(O)O—, —CH₂CH₂C(O)O—, —NR—, or —NRCH₂CH₂—. In some embodiments, L¹ is —CH₂—, —CH₂CH₂—, —CH₂CH₂CH₂—, —CH₂CH₂CH₂O—, —CH₂C(O)O—, —CH₂CH₂C(O)O—,

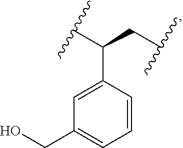 [SMILES: *C[C@H](*)c1cccc(CO)c1]

—N(CH₃)—, —CH(CH₃)—,

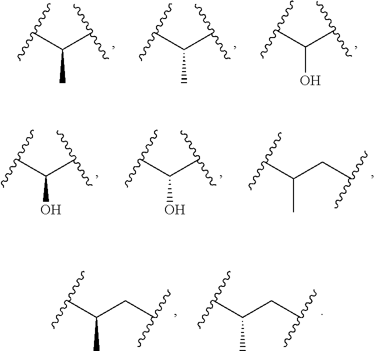

As described above and defined herein, each -Cy^{x}- is an optionally substituted ring selected from phenyl, a 3-7 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur.

In some embodiments, Cy^{x}- is an optionally substituted ring selected from phenyl, a 3-6 membered saturated or partially unsaturated carbocyclylenyl or heterocyclylenyl having 1-4 heteroatoms independently selected from nitrogen, oxygen, and sulfur, and a 5-9 membered monocyclic or bicyclic heteroarylenyl having 1-4 heteroatoms independently selected from nitrogen, oxygen, and sulfur.

In some embodiments, -Cy^{x}- is an optionally substituted phenyl. In some embodiments, -Cy^{x}- is an optionally substituted 3-6 membered saturated or partially unsaturated carbocyclylenyl. In some embodiments, -Cy^{x}- is an optionally substituted 3-6 membered saturated or partially unsaturated heterocyclylenyl having 1-4 heteroatoms independently selected from nitrogen, oxygen, and sulfur. In some embodiments, -Cy^{x}- is an optionally substituted 5-9 membered monocyclic or bicyclic heteroarylenyl having 1-4 heteroatoms independently selected from nitrogen, oxygen, and sulfur.

In some embodiments -Cy^{x}- is

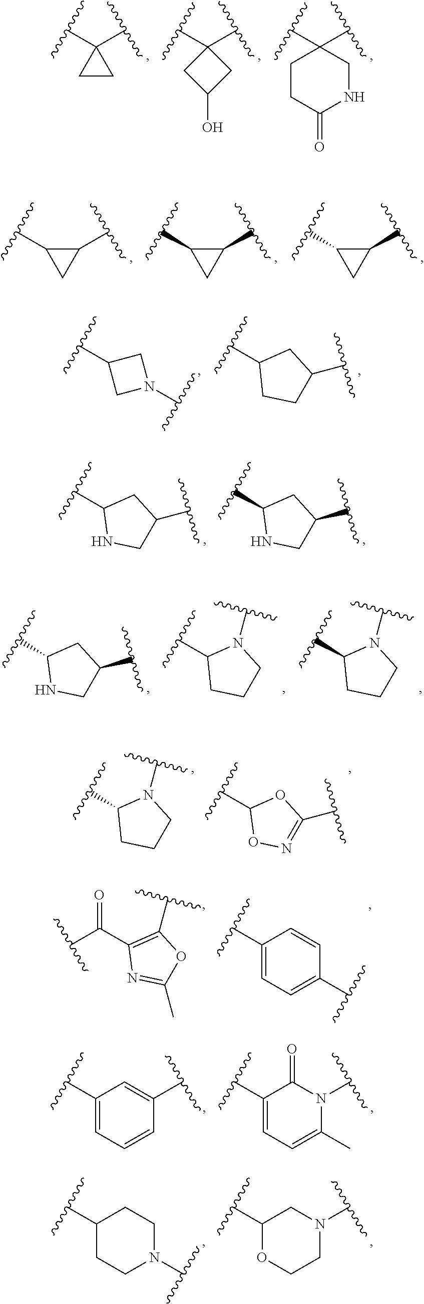

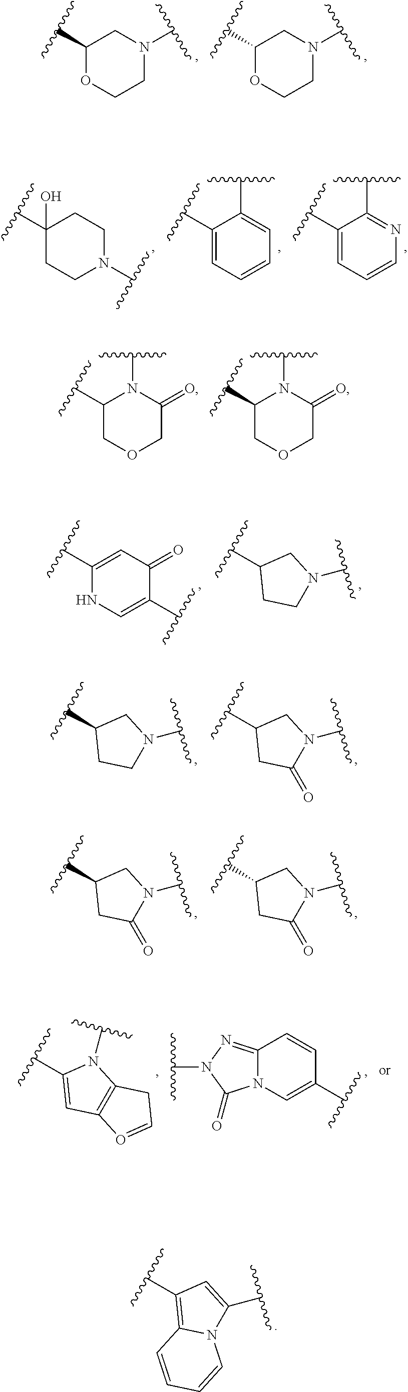

In some embodiment, -Cy^{x}- is as depicted in the compounds of Table 1B, below.

In some embodiments, L^{x} is —C(O)(CR₂)₁₋₃—. In some embodiments, L^{x} is —C(O)(CH₂)₁₋₃—.

In some embodiments, L^{x} is —C(O)—, —C(O)CH₂—, —S(O)₂CH₂—, —C(O)CH₂CH₂—, —C(O)OCH₂—, —C(O)CH₂O—, —C(O)CH₂CH₂CH₂—, —C(O)CH₂CH₂S(O)₂—, —C(O)CH₂CH₂CO₂—, —C(O)CH₂NHC(O)—, —C(O)CH₂N(Me)S(O)₂—,

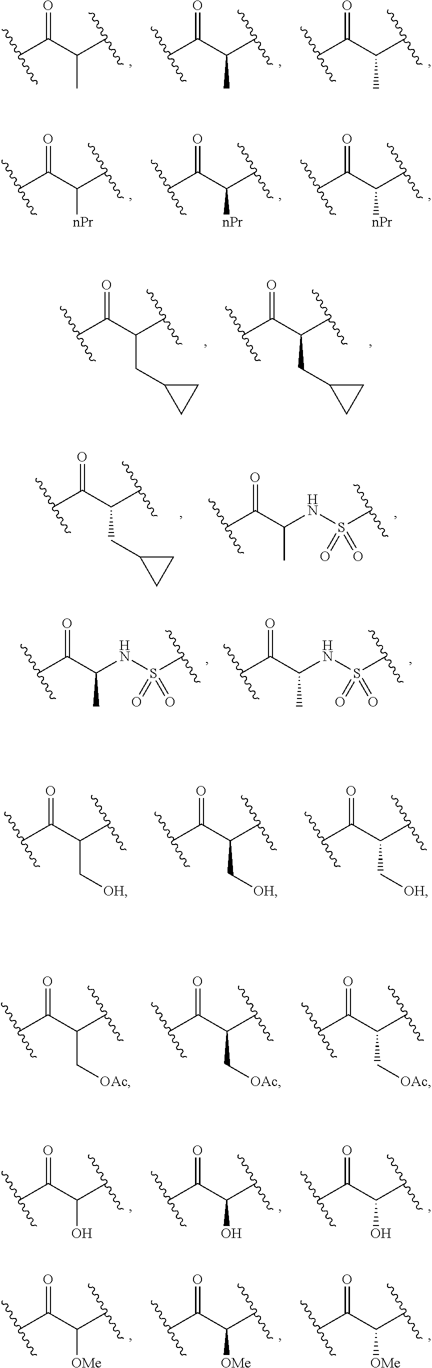

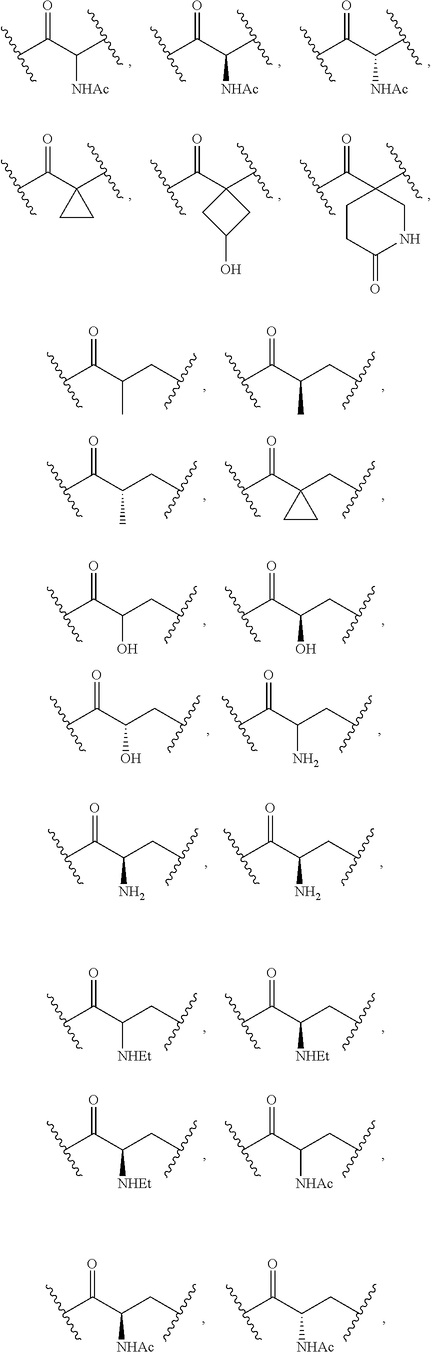

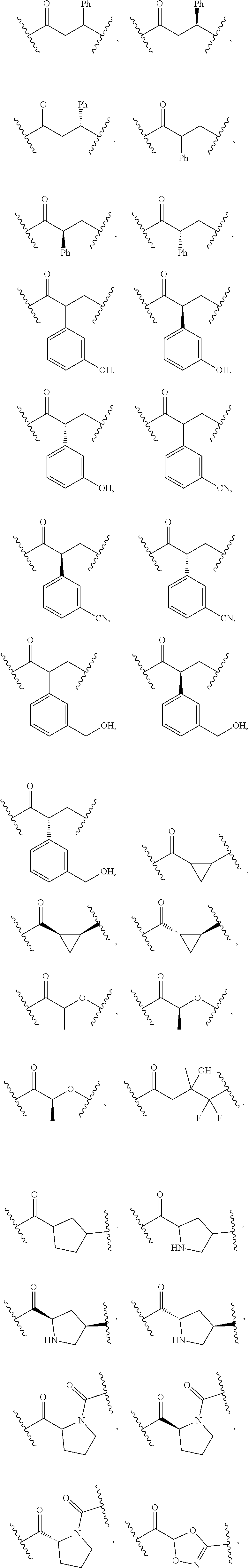

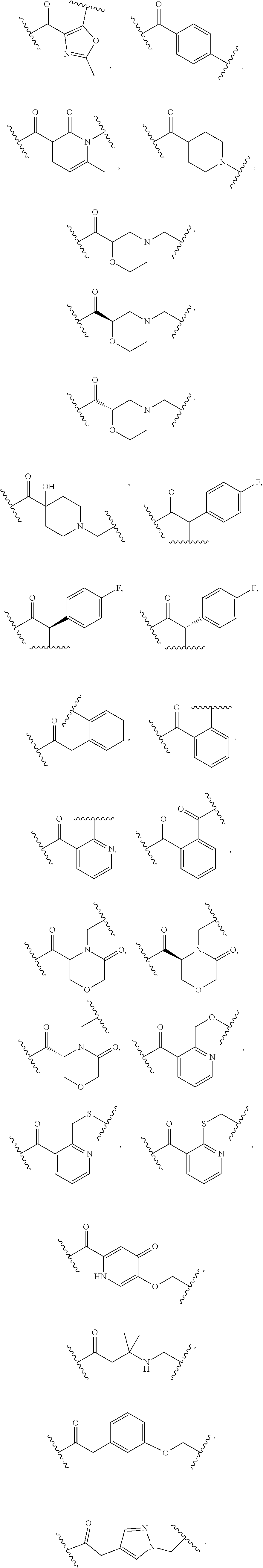

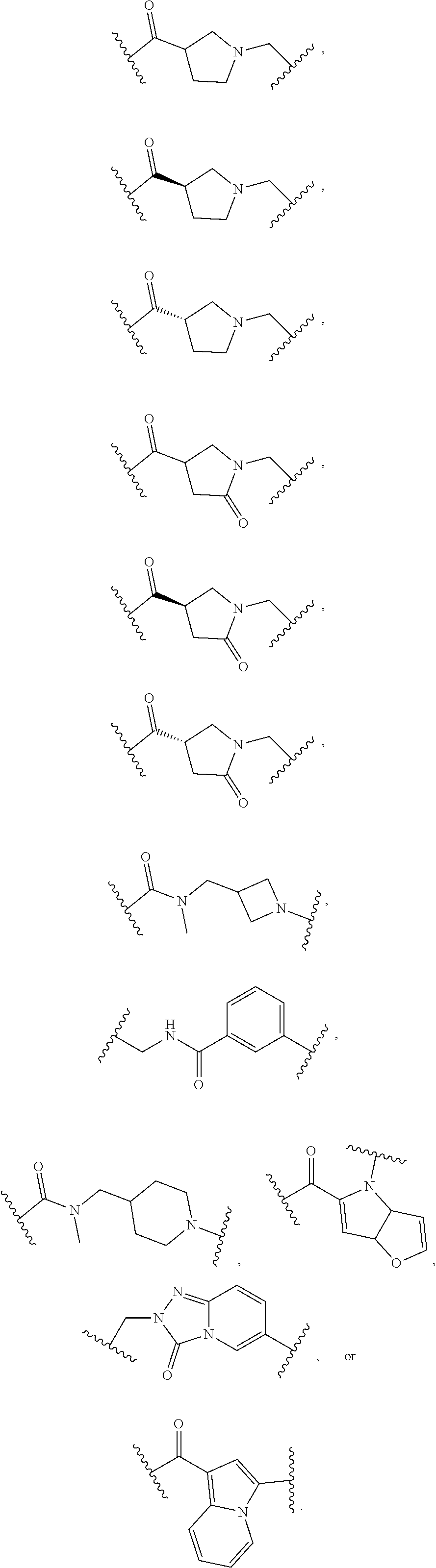

In some embodiments, L^{x} is

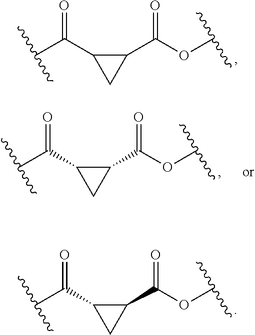

In some embodiments, L¹ is —S(O)₂CH₂CH₂—.

In some embodiment, L¹ is as depicted in the compounds of Table 1B, below.

In some embodiments, -Ring X-L^{x}- is:

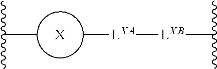

or a pharmaceutically acceptable salt thereof, wherein each of Ring X, L^{XA}, and L^{XB} is as defined above and described herein.

In some embodiments, -Ring X-L^{X}- is:

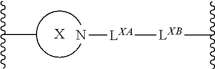

or a pharmaceutically acceptable salt thereof, wherein each of Ring X, L^{XA}, and L^{XB} is as defined above and described herein.

In some embodiments, -Ring X-L^{X}- is:

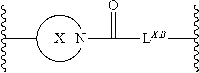

or a pharmaceutically acceptable salt thereof, wherein each of Ring X and L^{X} is as defined above and described herein.

As described above and defined herein, each of w, x, and y are independently 0, 1, 2, 3, or 4.

In some embodiments, w is 0. In some embodiments, w is 1. In some embodiments, w is 2. In some embodiments, w is 3. In some embodiments, w is 4. In some embodiments, w is 0 or 1. In some embodiments, w is 1 or 2.

In some embodiments, x is 0. In some embodiments, x is 1. In some embodiments, x is 2. In some embodiments, x is 3. In some embodiments, x is 4. In some embodiments, x is 0 or 1. In some embodiments, x is 1 or 2.

In some embodiments, y is 0. In some embodiments, y is 1. In some embodiments, y is 2. In some embodiments, y is 3. In some embodiments, y is 4. In some embodiments, y is 0 or 1. In some embodiments, y is 1 or 2.

In some embodiment, w, x, and y are as depicted in the compounds of Table 1B, below.

In some embodiments, SBM is as depicted in the compounds of Table 1B, below.

In certain embodiments, the present invention provides a compound of formula I as a compound of any one of the following formulae:

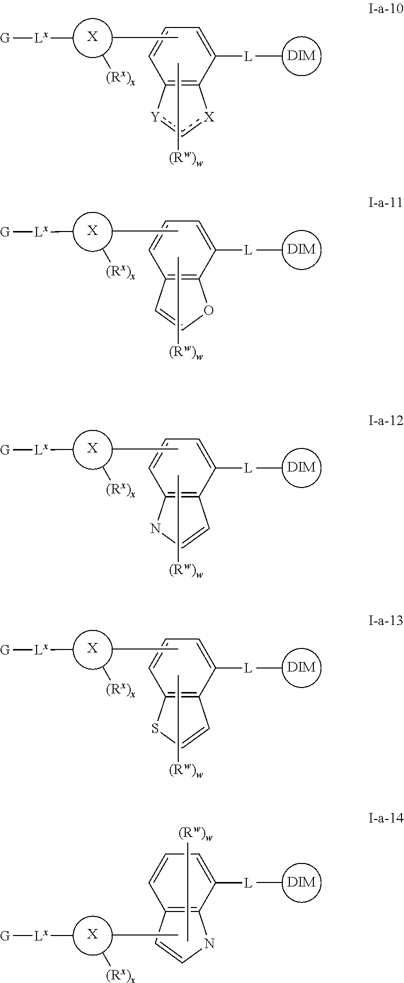

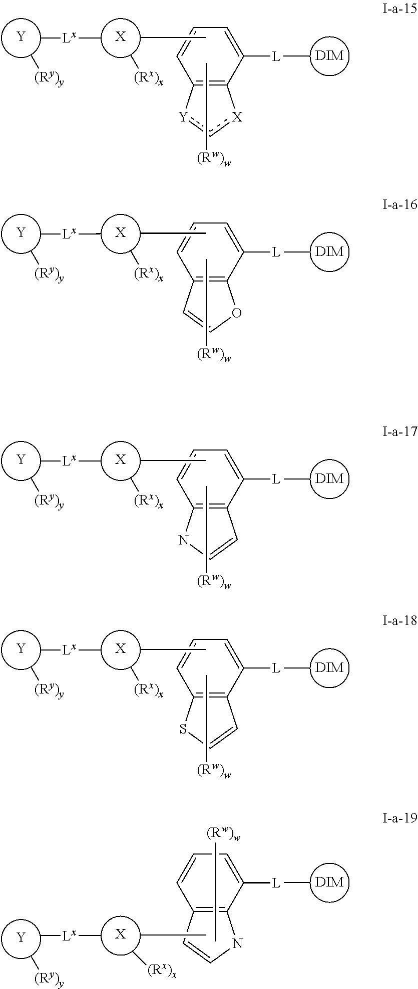

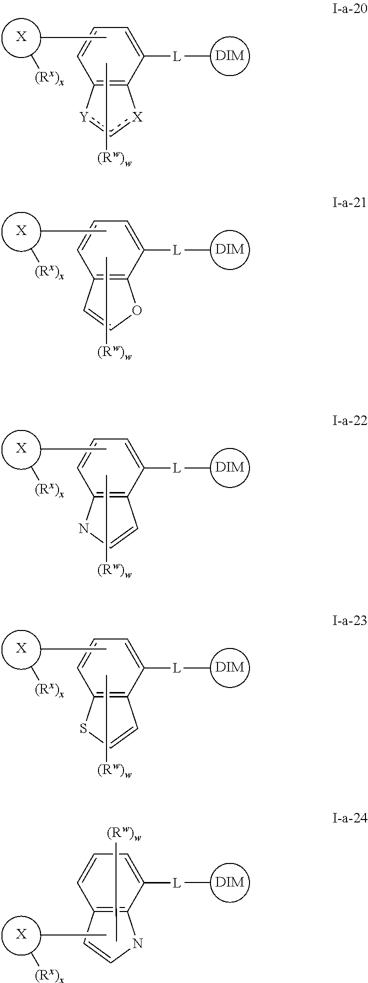

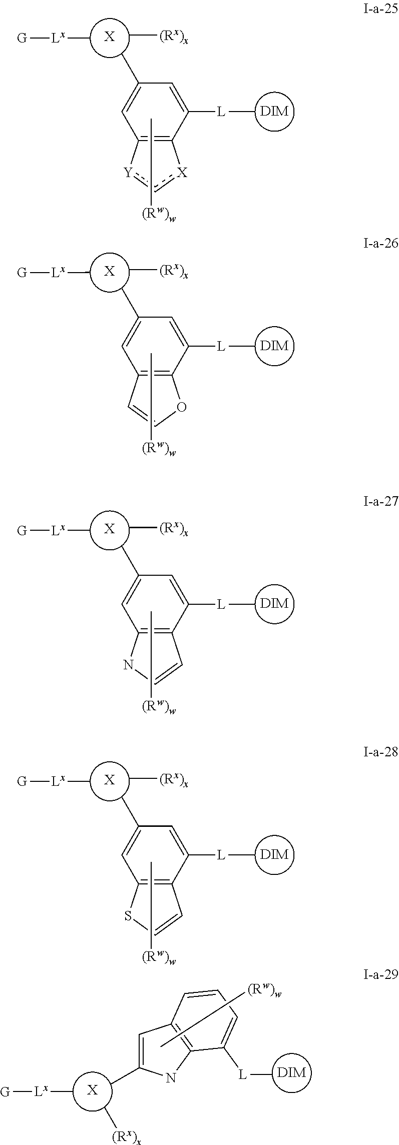

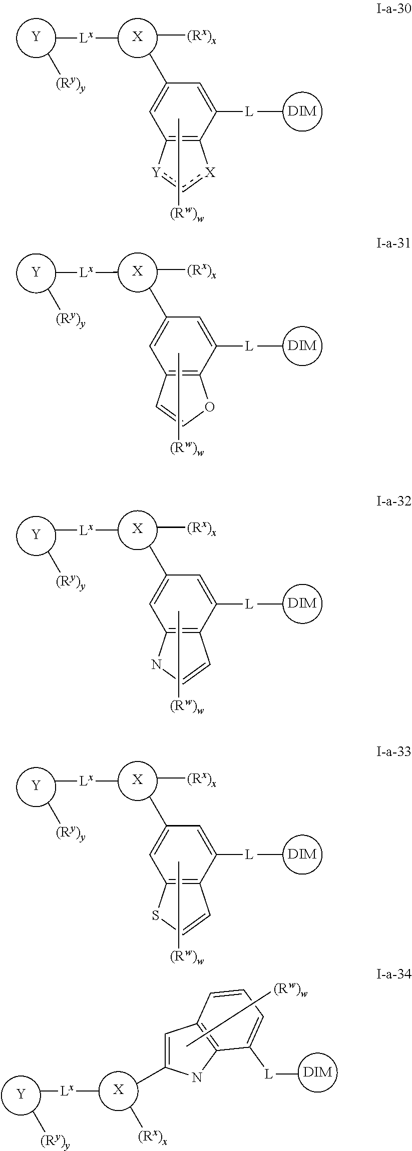

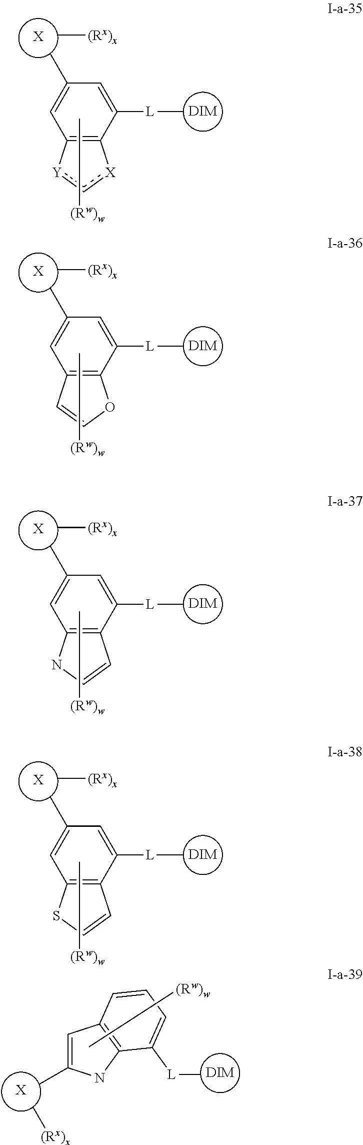

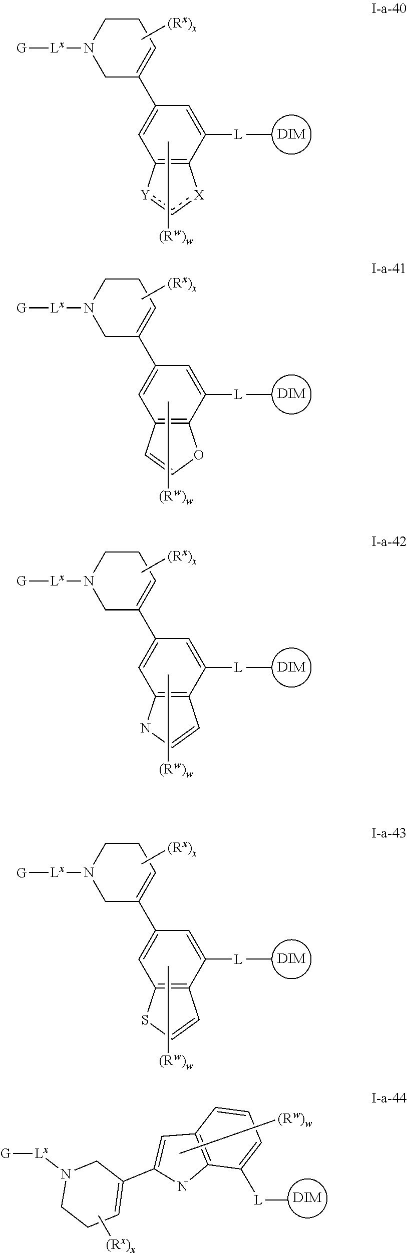

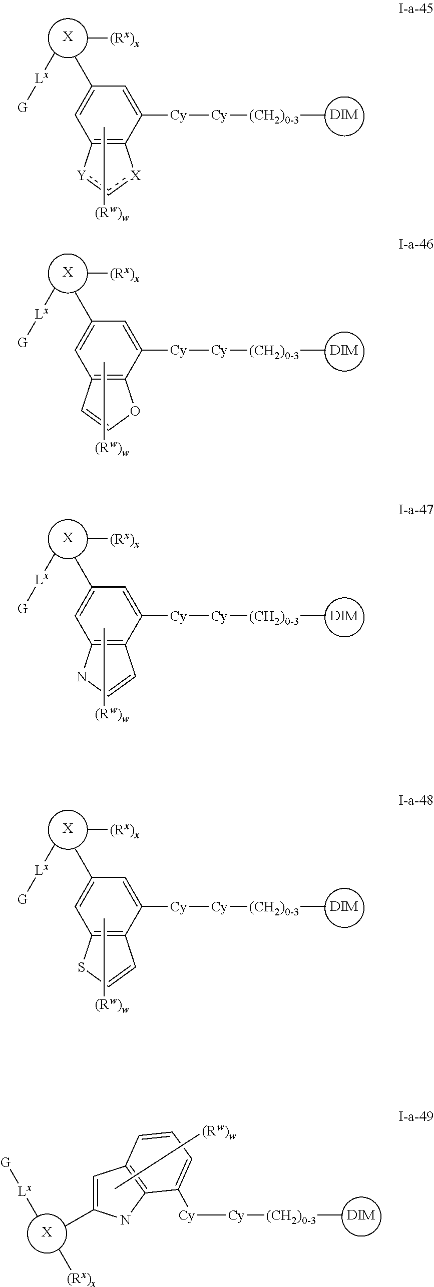

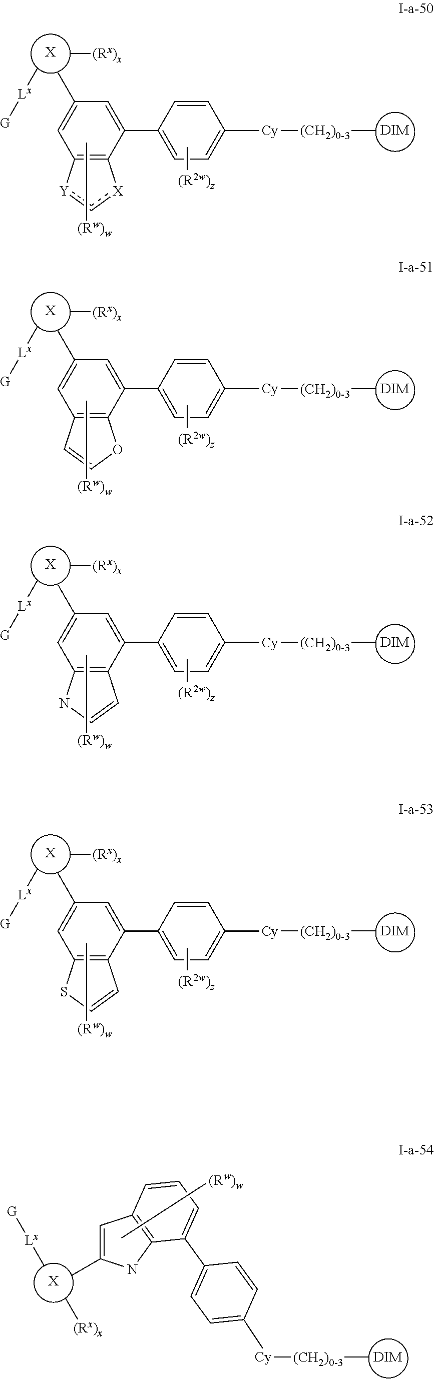

or a pharmaceutically acceptable salt thereof, wherein:
X is CH and Y is N—R^{w}, X is CH and Y is S, or X is O and Y is CH;
each R^{2w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R; and
z is 0, 1, 2, 3, or 4;
wherein DIM, L, -Cy-, Ring X, Ring Y, R, R^{w}, R^{x}, R^{y}, L^{x}, w, x, and y are as defined above and described herein both individually and in combination.

In some embodiments, X is CH and Y is N—R^{w}, X is CH and Y is S, or X is 0 and Y is CH.

In some embodiments, X is CH and Y is N—R^{w}. In some embodiments, X is CH and Y is NH. In some embodiments, X is CH and Y is S. In some embodiments, X is 0 and Y is CH.

In some embodiments, X and Y are as depicted in the compounds of Table 1B, below.

As described above and defined herein, R^{2w} is selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{2w} is hydrogen. In some embodiments, R^{2w} is R^{A}. In some embodiments, R^{2w} is halogen. In some embodiments, R^{2w} is —CN. In some embodiments, R^{2w} is —NO₂. In some embodiments, R^{2w} is —OR. In some embodiments, R^{2w} is —SR. In some embodiments, R^{2w} is —NR₂. In some embodiments, R^{2w} is —SiR₃. In some embodiments, R^{2w} is —S(O)₂R. In some embodiments, R^{2w} is —S(O)₂NR₂. In some embodiments, R^{2w} is —S(O)(NR)R. In some embodiments, R^{2w} is —S(O)R. In some embodiments, R^{2w} is —C(O)R. In some embodiments, R^{2w} is —C(O)OR. In some embodiments, R^{2w} is —C(O)NR₂. In some embodiments, R^{2w} is —C(O)NROR. In some embodiments, R^{2w} is —OC(O)R. In some embodiments, R^{2w} is —OC(O)NR₂. In some embodiments, R^{2w} is —P(O)R₂. In some embodiments, R^{2w} is —P(O)(OR)₂. In some embodiments, R^{2w} is —OP(O)R₂. In some embodiments, R^{2w} is —OP(O)(OR)₂. In some embodiments, R^{2w} is —OP(O)(OR)NR₂. In some embodiments, R^{2w} is —OP(O)(NR₂)₂. In some embodiments, R^{2w} is —NRC(O)OR. In some embodiments, R^{2w} is —NRC(O)R. In some embodiments, R^{2w} is —NRC(O)N(R)₂. In some embodiments, R^{2w} is —NP(O)R₂. In some embodiments, R^{2w} is —NRP(O)(OR)₂. In some embodiments, R^{2w} is —NRP(O)(OR)NR₂. In some embodiments, R^{2w} is —NRP(O)(NR₂)₂. In some embodiments, R^{2w} is —NRS(O)₂R.

In some embodiments, each R^{2w} is independently hydrogen, C₁₋₆ alkyl, C₁₋₆ haloalkyl, halogen. —OC₁₋₆alkyl, or —OC₁₋₆haloalkyl.

In some embodiments, each R² is independently fluoro, chloro, methyl, ethyl, —CHF₂, —CMeF₂, —CF₃, —OMe, —OEt, —OCHF₂, —OCMeF₂, or —OCF₃.

In some embodiments, R^{2w} is fluoro. In some embodiments, R^{2w} is chloro. In some embodiments, R^{2w} is fluoro and chloro. In some embodiments, R^{2w} is —OMe.

In some embodiments, R^{2w} is as depicted in the compounds of Table 1B, below.

As described above and defined herein, z is 0, 1, 2, 3, or 4.

In some embodiments, z is 0. In some embodiments, z is 1. In some embodiments, z is 2. In some embodiments, z is 3. In some embodiments, z is 4. In some embodiments, z is 0 or 1. In some embodiments, z is 1 or 2.

In some embodiment, z is as depicted in the compounds of Table 1B, below.

In certain embodiments, the present invention provides a compound of formula I-a″″-1 as any one of the following formulae:

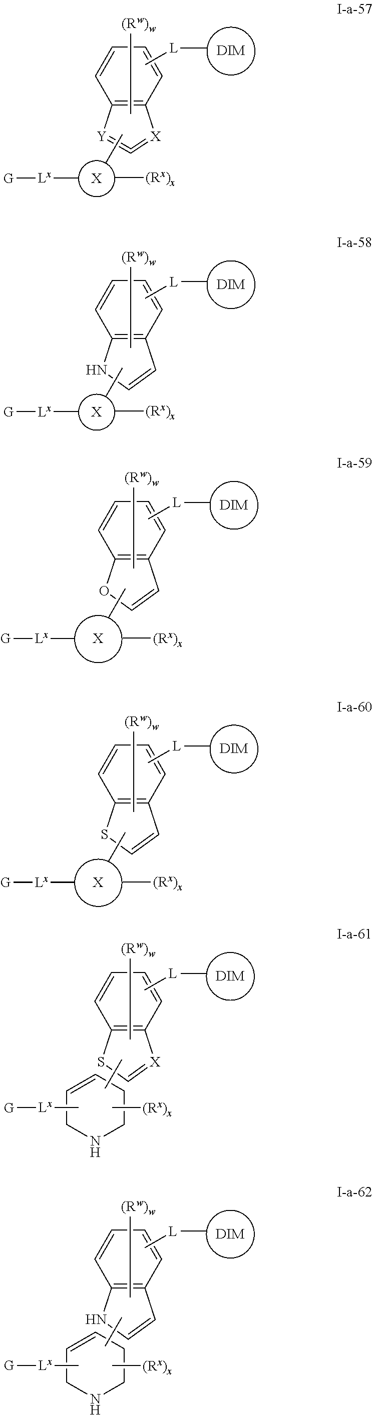

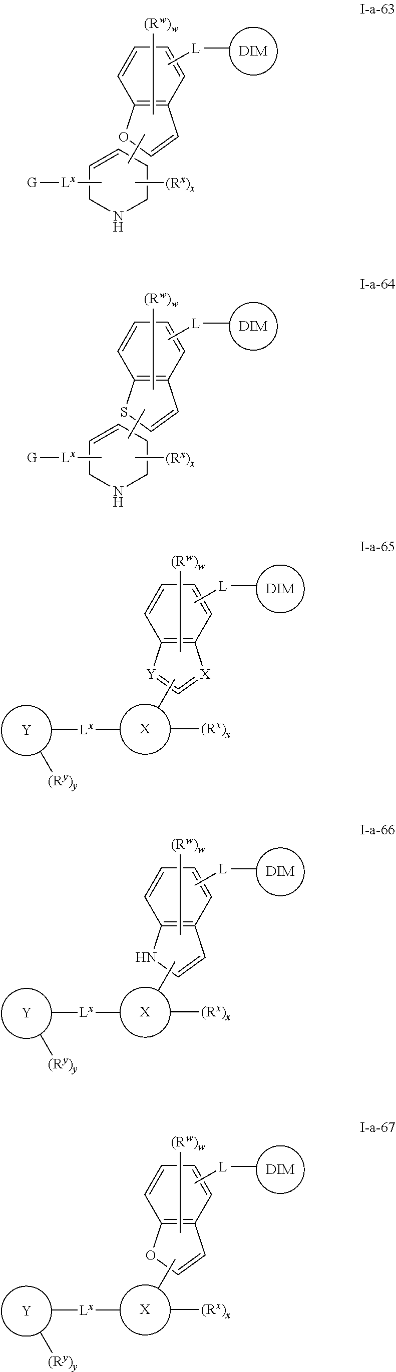

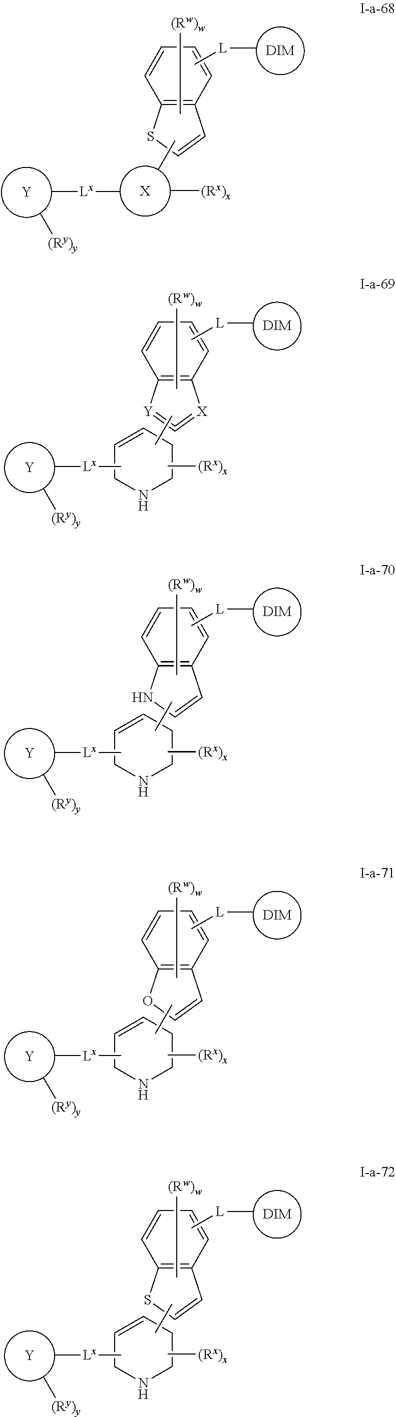

or a pharmaceutically acceptable salt thereof, wherein each of the variables are as defined above and described herein both individually and in combination, and wherein:
each of X and Y of Ring W is independently N, NH, N—R^{W}, —O—, —S—, C—H, C—R^{W}, C—H₂, CH(R^{W}), or C—(R^{W})₂.

### Ligase Binding Moiety (LBM)

In some embodiments, DIM is LBM. In some embodiments, LBM is an E3 ligase ligand. In some embodiments, LBM comprises means for binding an E3 ubiquitin ligase. In some embodiments, LBM comprises means for binding a cereblon E3 ubiquitin ligase. As defined herein and described below, wherein a formula is depicted using square brackets, e.g.,

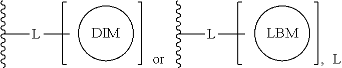

is attached to a modifiable carbon, oxygen, or nitrogen atom within DIM or LBM including substitution or replacement of a defined group in DIM or LBM. In certain embodiments, the present invention provides a compound of formula I, wherein LBM is a compound of formula I-aa:

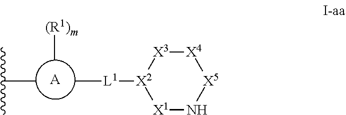

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

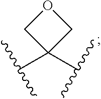 [SMILES: *C1(*)COC1]

X² is N, C—R^{B}, Si—R^{B}, or P═O;
X³ and X⁴ are independently a covalent bond, —CR₂—, —CF₂—, —O—, —S—, or X³—X⁴ is —CR═CR—;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₆ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur;
m is 0, 1, 2, 3, 4, or 5.

In certain embodiments, the present invention provides a compound of formula I, wherein LBM is of formula I-aa′:

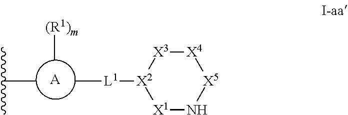

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

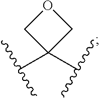 [SMILES: *C1(*)COC1]

X² is N, C—R^{B}, Si—R^{B}, or P═O:
X³ and X⁴ are independently a covalent bond, —CR₂—, —CF₂—, —O—, —S—, or X³—X⁴ is —CR═CR—;
each R¹ is independently —H, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂. —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R: or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo: or a 5-10 membered heteroaryl having 1-4 heteroatoms independently selected from nitrogen, oxygen, or sulfur;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂:
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In certain embodiments, the present invention provides a compound of formula I, wherein LBM is a compound of formula I-aa-1:

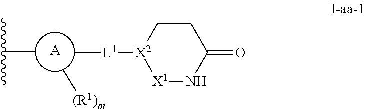

or a pharmaceutically acceptable salt thereof, wherein:
X¹ is a bivalent moiety selected from —CH₂— or —C(O)—:
X² is N or CH:
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂:
Ring A is a ring selected from phenylenyl, naphthalenyl, pyridinylenyl,

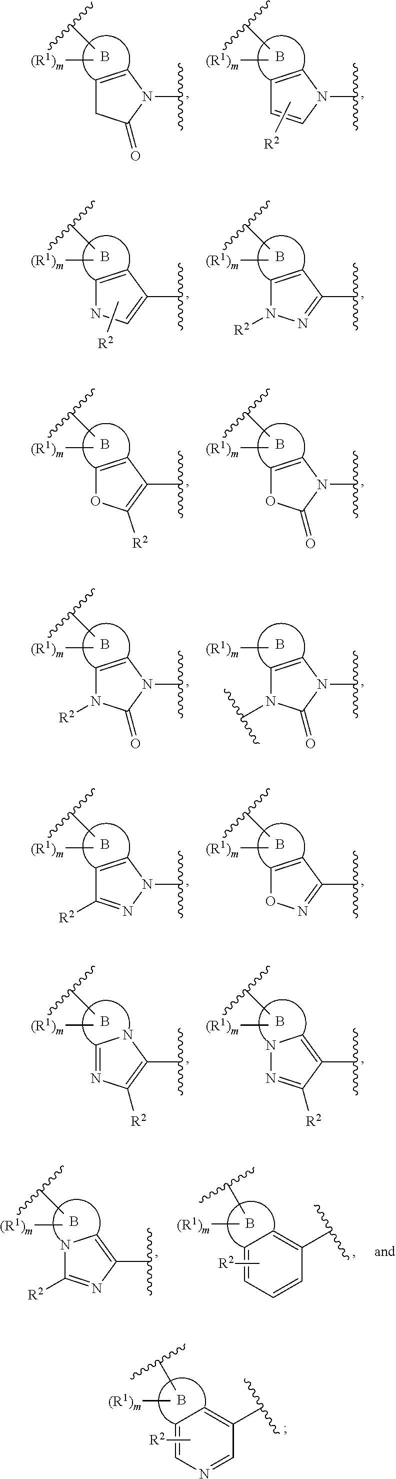

Ring B is a fused ring selected from benzo or a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR—N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R;
R² is hydrogen, halogen. C₁₋₆ alkyl, C₃₋₆ cycloalkyl, C₁₋₆ haloalkyl, —OC₁₋₆ alkyl, —OC₃₋₆ cycloalkyl, or —OC₁₋₆ haloalkyl;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

In certain embodiments, the present invention provides a compound of formula I, wherein LBM is of formula I-aa-1′:

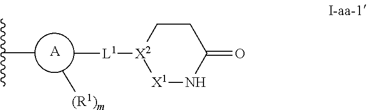

or a pharmaceutically acceptable salt thereof, wherein:
X¹ is a bivalent moiety selected from —CH₂— or —C(O)—:
X² is N or CH:
L¹ is a covalent bond or a C₁-3 bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is a ring selected from phenylenyl, naphthalenyl, pyridinylenyl,

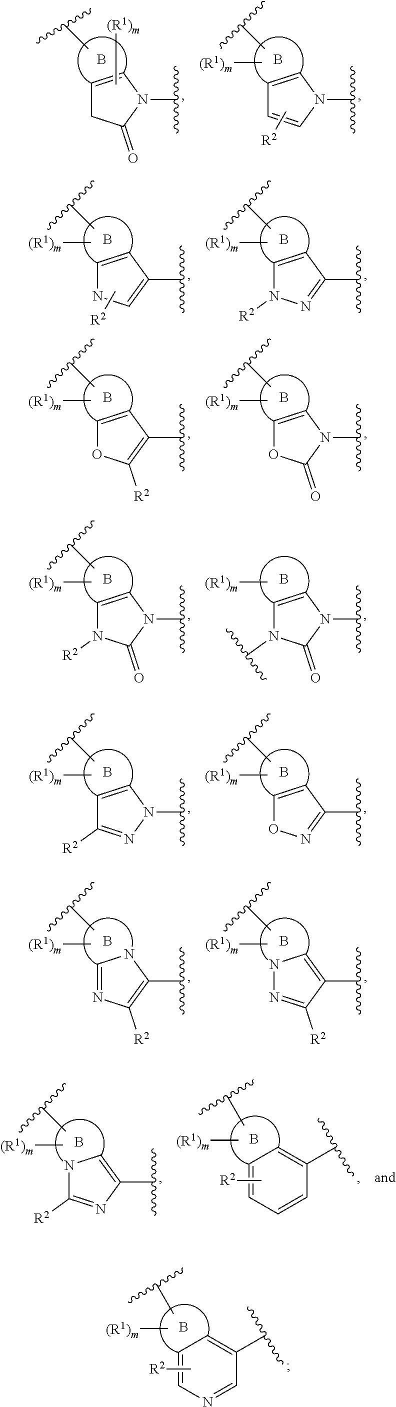

Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR—N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R: or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 3-10 membered heteroaryl having 1-4 heteroatoms independently selected from nitrogen, oxygen, or sulfur;
R² is hydrogen, halogen. C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl; or:
   an R² and an R are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

For formula I-aa-1 and I-aa-1′, it will be appreciated that an occurrence of R² reduces the available occurrences of m by 1.

In some embodiments, the present invention provides a compound of formula I-aa-1 as a compound of any one of the following formulae:

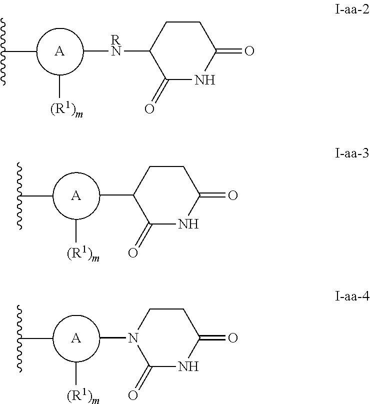

or a pharmaceutically acceptable salt thereof.

In some embodiments, the present invention provides a compound of formula I-aa′ or I-aa-1′ as a compound of any one of the following formulae:

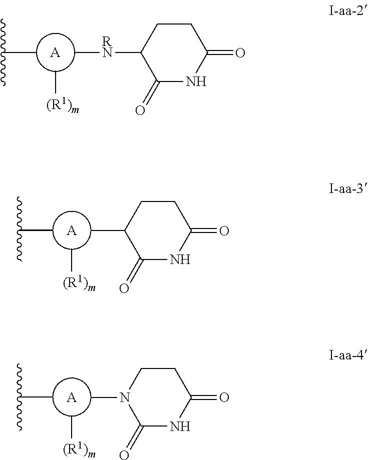

or a pharmaceutically acceptable salt thereof, wherein each R¹, m, Ring A, and R is as defined above in I-aa′ or I-aa-1′ and described herein both individually and in combination.

In some embodiments, the present invention provides a compound of formula I-aa′ or I-aa-1′ as a compound of any one of the following formulae:

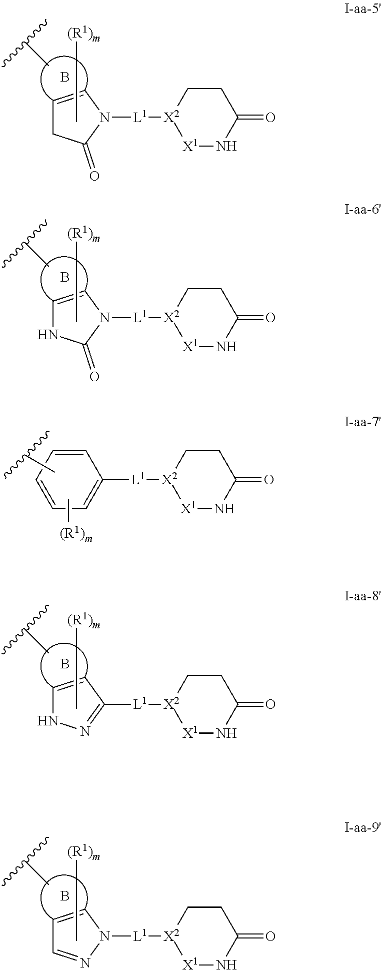

or a pharmaceutically acceptable salt thereof, wherein each L¹, R¹, m, X¹, X², and Ring B is as defined above in I-aa′ or I-aa-1′ and described herein both individually and in combination.

In some embodiments, the present invention provides a compound of formula I-aa′ or I-aa-1′ as a compound of any one of the following formulae:

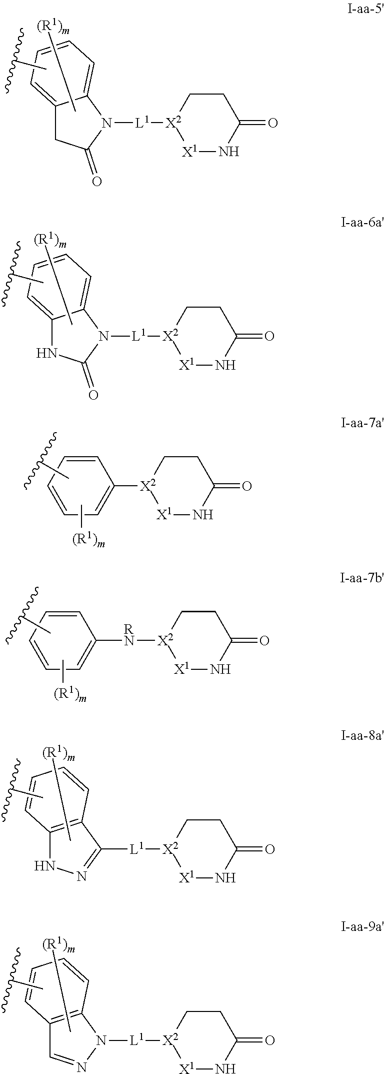

or a pharmaceutically acceptable salt thereof, wherein each L¹, R¹, n, X¹, and X² is as defined above in I-aa′ or I-aa-1′ and described herein both individually and in combination.

In some embodiments, the present invention provides a compound of formula I-aa′ or I-aa-1′ as a compound of any one of the following formulae:

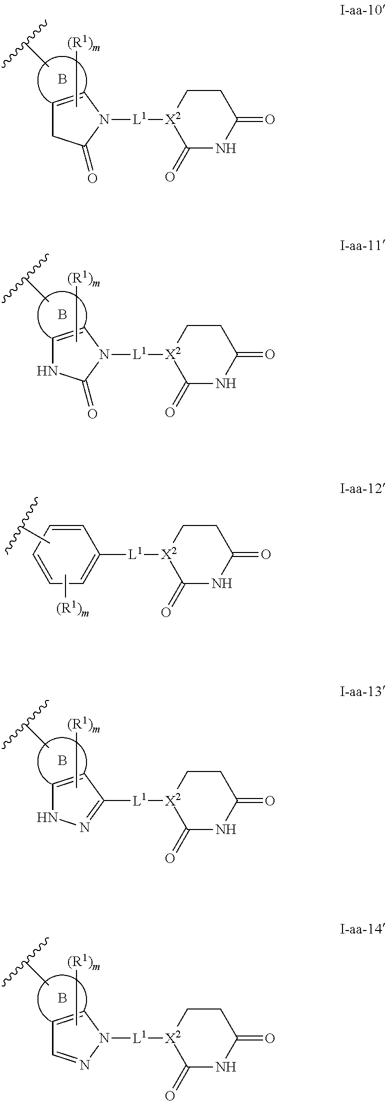

or a pharmaceutically acceptable salt thereof, wherein each variable is as defined above and described herein both individually and in combination.

In some embodiments, the present invention provides a compound of formula I-aa′ or I-aa-1′ as a compound of any one of the following formulae:

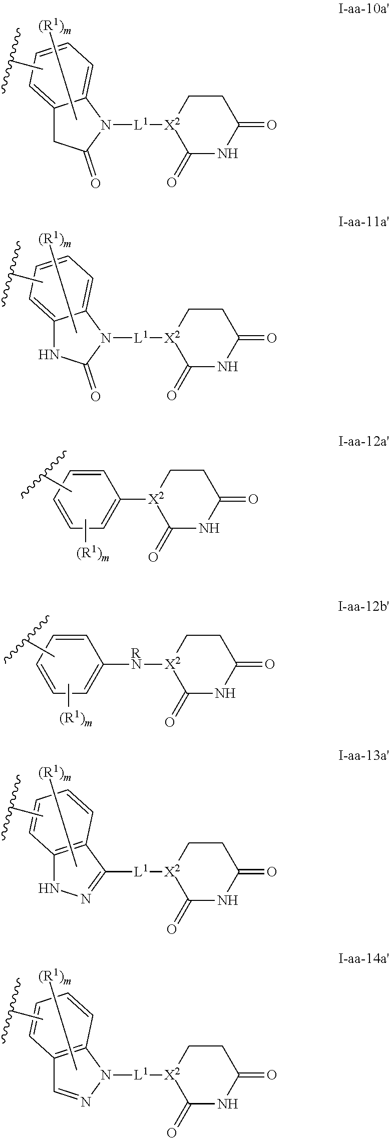

or a pharmaceutically acceptable salt thereof, wherein each variable is as defined above and described herein both individually and in combination.

As defined above and described herein, X¹ and X⁵ are independently a covalent bond, —CR₂—, —SO₂—, —S(O)—, —P(O)R—, —P(O)OR—, —P(O)N(R)₂—, —C(O)—, —C(S)—, or

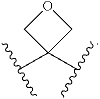 [SMILES: *C1(*)COC1]

In some embodiments, X¹ is a covalent bond. In some embodiments, X¹ is —CR₂—. In some embodiments, X¹ is —SO₂—. In some embodiments, X¹ is —S(O)—. In some embodiments, X¹ is —P(O)R—. In some embodiments, X¹ is —P(O)OR—. In some embodiments, X¹ is —P(O)N(R)₂—. In some embodiments, X¹ is —C(O)—. In some embodiments, X¹ is —C(S)—, or

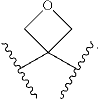 [SMILES: *C1(*)COC1]

In some embodiments, X¹ is —CH₂—. In some embodiments, X¹ is —C(O)—.

In some embodiments, X¹ is selected from those depicted in the compounds of Table 1B below.

In some embodiments, X⁵ is a covalent bond. In some embodiments, X⁵ is —CR₂—. In some embodiments, X⁵ is —SO₂—. In some embodiments, X⁵ is —S(O)—. In some embodiments, X⁵ is —P(O)R—. In some embodiments, X⁵ is —P(O)OR—. In some embodiments, X⁵ is —P(O)N(R)₂—. In some embodiments, X⁵ is —C(O)—. In some embodiments, X⁵ is —C(S)—, or

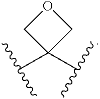 [SMILES: *C1(*)COC1]

In some embodiments, X⁵ is —CH₂—. In some embodiments, X⁵ is —C(O)—.

In some embodiments, X⁵ is selected from those depicted in the compounds of Table 1B below.

As defined above and described herein, X² is N, C—R^{B}, Si—R^{B}, or P═O. In some embodiments, X² is N. In some embodiments, X² is C—Re. In some embodiments, X² is Si—R^{B}. In some embodiments, X² is P═O. In some embodiments, X² is CH.

In some embodiments, X² is selected from those depicted in the compounds of Table 1B below.

As defined above and described herein, X³ and X⁴ are independently a covalent bond, —CR₂—, —CF₂—, —O—, —S—, or X³-X⁴ is —CR═CR—. In some embodiments, X³ is —CR₂—. In some embodiments, X¹ is —CF₂—. In some embodiments, X³ is

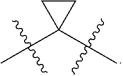 [SMILES: *C1(*)CC1]

In some embodiments, X³ is —O—. In some embodiments, X¹ is —S—.

In some embodiments, X³ is selected from those depicted in the compounds of Table 1B below.

In some embodiments, X⁴ is —CR₂—. In some embodiments, X⁴ is —CF₂—. In some embodiments, X⁴ is

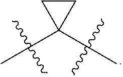 [SMILES: *C1(*)CC1]

In some embodiments, X⁴ is —O—. In some embodiments, X⁴ is —S—.

In some embodiments, X³-X⁴ is —CR═CR—.

In some embodiments, X⁴ is selected from those depicted in the compounds of Table 1B below.

As defined above and described herein, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R: or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R.

In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, R¹ is R^{A}. In some embodiments, R¹ is —CN. In some embodiments, R¹ is —NO₂. In some embodiments, R¹ is —OR. In some embodiments, one or more of R¹ is —Si(OH)₂R. In some embodiments, R¹ is —Si(OH)R₂. In some embodiments, R¹ is —SR. In some embodiments, R¹ is —NR₂. In some embodiments, R¹ is —SiR₃. In some embodiments, R¹ is —S(O)₂R. In some embodiments, R¹ is —S(O)₂NR₂. In some embodiments, R¹ is —S(O)R. In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —CR^{Z}N(R)C(O)R. In some embodiments, R¹ is -CR₂N(R)C(O)NR₂. In some embodiments, R¹ is —CFR₂. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —OC(O)R. In some embodiments, R¹ is —OC(O)NR₂. In some embodiments, R¹ is —OP(O)R₂. In some embodiments, R¹ is —OP(O)(OR)₂. In some embodiments, R¹ is —OP(O)(OR)NR₂. In some embodiments, R¹ is independently —OP(O)(NR₂)₂—. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)P(O)R₂. In some embodiments, R¹ is —N(R)P(O)(OR)₂. In some embodiments, R¹ is —N(R)P(O)(OR)NR₂. In some embodiments, R¹ is —N(R)P(O)(NR₂)₂. In some embodiments, R¹ is —N(R)S(O)₂R.

In some embodiments, R¹ is halogen, C₁₋₆alkyl. —OC₁₋₆alkyl, C₁₋₆ haloalkyl, —OC₁₋₆ alkyl, or —OC₁₋₆ haloalkyl.

In some embodiments, R¹ is hydrogen. In some embodiments, R¹ is fluoro. In some embodiments, R¹ is chloro. In some embodiments, R¹ is methyl. In some embodiments, R¹ is —C(OH)Me₂. In some embodiments, R¹ is —CHF₂. In some embodiments, R¹ is —CF₃. In some embodiments, R¹ is —OMe.

In some embodiments, two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R¹ groups of Ring A are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, two R¹ groups of Ring A are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R¹ groups of Ring A are taken together with their intervening atoms to form an optionally substituted benzo.

In some embodiments, two R¹ groups of Ring A are taken together with their intervening atoms to form an optionally substituted ring selected from a 5-10 membered heteroaryl having 1-4 heteroatoms independently selected from nitrogen, oxygen, or sulfur. In some embodiments, two R¹ groups of Ring A are taken together with their intervening atoms to form an optionally substituted ring selected from a 5-6 membered heteroaryl having 1-4 heteroatoms independently selected from nitrogen, oxygen, or sulfur.

In some embodiments, two R¹ groups on the same atom of Ring A are taken together to form an optionally substituted ring selected from a 3-10 membered saturated or partially unsaturated carbocyclyl or heterocyclyl having 1-4 heteroatoms independently selected from nitrogen, oxygen, or sulfur; benzo. In some embodiments, two R¹ groups on the same atom of Ring A are taken together to form an optionally substituted ring selected from a 3-10 membered saturated or partially unsaturated carbocyclyl. In some embodiments, two R¹ groups on the same atom of Ring A are taken together to form an optionally substituted ring selected from a 3-6 membered saturated or partially unsaturated carbocyclyl. In some embodiments, two R¹ groups on the same atom of Ring A are taken together to form an optionally substituted ring selected from a 3 membered saturated or partially unsaturated carbocyclyl. In some embodiments, two R¹ groups on the same atom of Ring A are taken together to form an optionally substituted ring selected from a 3-10 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3-6 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted cyclopropyl ring.

In some embodiments, R¹ is selected from those depicted in the compounds of Table 1B below.

As defined above and described herein, R² is hydrogen, halogen. C₁₋₆ alkyl, C₃₋₆ cycloalkyl, C₁₋₆ haloalkyl, —OC₁₋₆ alkyl, —OC₃₋₆ cycloalkyl, or —OC₁₋₆ haloalkyl: or: two R² groups are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, R² is hydrogen, halogen, C₁₋₆ alkyl, C₃₋₆ cycloalkyl, C₁₋₆ haloalkyl, —OC₃₋₆ alkyl, —OC₃₋₆ cycloalkyl, or —OC₁₋₆ haloalkyl.

In some embodiments, R² is hydrogen. In some embodiments, R² is halogen. In some embodiments, R² is C₁₋₆ alkyl. In some embodiments, R² is C₃₋₆ cycloalkyl. In some embodiments, R² is C₁₋₆ haloalkyl. In some embodiments, R² is —OC₁₋₆ alkyl. In some embodiments, R² is —OC₃₋₆ cycloalkyl. In some embodiments, R² is —OC₁₋₆ haloalkyl.

In some embodiments, R² is methyl. In some embodiments, R² is ethyl. In some embodiments, R² is cyclopropyl.

In some embodiments, an R² and an R¹ group are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, an R² and an R¹ group are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, an R² and an R¹ group are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, an R² and an R¹ group are taken together with their intervening atoms to form an optionally substituted benzo.

In some embodiments, an R² and an R¹ group are taken together with their intervening atoms to form an optionally substituted ring selected from a 5-10 membered heteroaryl having 1-4 heteroatoms independently selected from nitrogen, oxygen, or sulfur. In some embodiments, an R² and an R¹ group are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in the compounds of Table 1B below.

As defined above and described herein, each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R^{A} is an optionally substituted C₁₋₆ aliphatic. In some embodiments, each R^{A} is an optionally substituted phenyl. In some embodiments, each R^{A} is an optionally substituted 3-10 membered saturated or partially unsaturated carbocyclic ring. In some embodiments, each R^{A} is an optionally substituted 3-10 membered heterocyclic ring having 1-2 heteroatoms independently selected from nitrogen, oxygen, and sulfur. In some embodiments, each R^{A} is an optionally substituted 5-6 membered heteroaryl ring having 1-4 heteroatoms independently selected from nitrogen, oxygen, and sulfur.

As defined above and described herein, each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic.

In some embodiments, R is hydrogen. In some embodiments, R is halogen. In some embodiments, R^{B} is —CN. In some embodiments, R^{B} is —OR. In some embodiments, R^{B} is —SR. In some embodiments, R^{B} is —S(O)R. In some embodiments, R^{B} is —S(O)₂R. In some embodiments, R^{B} is —NR₂In some embodiments, R^{B} is —P(O)(OR)₂. In some embodiments, R^{B} is —P(O)(NR₂)OR. In some embodiments, R^{B} is —P(O)(NR₂)₂. In some embodiments, R^{B} is —Si(OH)₂R. In some embodiments, R^{B} is —Si(OH)R₂. In some embodiments, R^{B} is —SiR₃. In some embodiments, R^{B} is an optionally substituted C₁₋₄ aliphatic.

In some embodiments, R^{B} is C₁₋₄ aliphatic optionally substituted with 1-3 halogens. In some embodiments, R^{B} is C₁₋₄ aliphatic. In some embodiments, R^{B} is methyl. In some embodiments, R^{B} is fluoro.

In some embodiments, R^{B} is selected from those depicted in the compounds of Table 1B below.

As defined above and described herein, L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L¹ is a covalent bond. In some embodiments, L¹ is a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L¹ is a covalent bond, —O—, —NR—, —S—, —CR₂—, —NRC(O)—, or —C(O)NR—. In some embodiments, L¹ is —O—, —NR—, —S—, —CR₂—, —NRC(O)—, or —C(O)NR—. In some embodiments, L¹ is —O—. In some embodiments, L¹ is —NR—. In some embodiments, L¹ is —S—. In some embodiments, L¹ is —CR₂—. In some embodiments, L¹ is —CH₂—. In some embodiments, L¹ is —NRC(O)—. In some embodiments, L¹ is —C(O)NR—. In some embodiments, L¹ is —NHC(O)—. In some embodiments, L¹ is —C(O)NH—.

In some embodiments, L¹ is selected from those depicted in the compounds of Table 1B below.

As defined above and described herein, Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur.

In some embodiments, Ring A is a phenylenyl. In some embodiments, Ring A is a naphthalenyl. In some embodiments, Ring A is pyridinylenyl. In some embodiments, Ring A is a 4-7 membered saturated or partially unsaturated carbocyclylenyl. In some embodiments, Ring A is a 4-7 membered saturated or partially unsaturated heterocyclylenyl having 1-3 heteroatoms independently selected from nitrogen, oxygen, and sulfur. In some embodiments, Ring A is an 8-10 membered bicyclic saturated or partially unsaturated heterocyclylenyl having 1-2 heteroatoms independently selected from nitrogen, oxygen, and sulfur. In some embodiments, Ring A is a 5-6 membered heteroarylenyl having 1-4 heteroatoms independently selected from nitrogen, oxygen, and sulfur. In some embodiments, Ring A is an 8-10 membered bicyclic heteroarylenyl having 1-5 heteroatoms independently selected from nitrogen, oxygen, and sulfur. In some embodiments, Ring A is an 8-15 membered saturated or partially unsaturated tricyclic heterocyclylenyl having 1-5 heteroatoms independently selected from nitrogen, oxygen, and sulfur. In some embodiments, Ring A is or an 8-15 membered tricyclic heteroarylenyl having 1-5 heteroatoms independently selected from nitrogen, oxygen, and sulfur.

In some embodiments, Ring A is a 9- or 10-membered saturated or partially unsaturated monocyclic or bicyclic heterocyclylenyl or heteroarylenyl containing 1-4 heteroatoms independently selected from nitrogen, oxygen, and sulfur.

In some embodiments, Ring A is an 8-10 membered saturated or partially unsaturated bicyclic heterocyclylenyl or heteroarylenyl containing 1-4 heteroatoms independently selected from nitrogen, oxygen, and sulfur. In some embodiments, Ring A is a 9- or 10-membered saturated or partially unsaturated bicyclic heterocyclylenyl or heteroarylenyl containing 1-4 heteroatoms independently selected from nitrogen, oxygen, and sulfur.

In some embodiments, Ring A is a 9-membered saturated or partially unsaturated bicyclic heterocyclylenyl or heteroarylenyl containing 1-4 heteroatoms independently selected from nitrogen, oxygen, and sulfur. In some embodiments, Ring A is a 9-membered saturated or partially unsaturated bicyclic heterocyclylenyl or heteroarylenyl containing 1 nitrogen and 1 oxygen heteroatom.

In some embodiments, Ring A is a 5,6-fused saturated or partially unsaturated bicyclic heterocyclylenyl or heteroarylenyl containing 1-4 heteroatoms independently selected from nitrogen, oxygen, and sulfur. In some embodiments, Ring A is a 5,6-fused saturated or partially unsaturated bicyclic heterocyclylenyl or heteroarylenyl containing 1 nitrogen and 1 oxygen heteroatom.

In some embodiments, Ring A is not phthalimide.

In some embodiments, Ring A is:

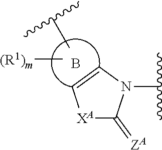

or a pharmaceutically acceptable salt thereof, wherein each R¹, Ring B, X^{A}, and in is as defined above and described herein both individually and in combination; and:
X^{A} is CH₂, CHR¹, C(R¹)₂, NH, NR¹, O or S. Z^{A} is O, S, or NR.

As defined above and described herein, X^{A} is CH₂, CHR¹, C(R¹)₂, NH, NR¹, O or S. In some embodiments, X^{A} is CH₂. In some embodiments, X^{A} is CHR¹. In some embodiments, X^{A} is C(R¹)₂, NH, NR¹, O or S.

In some embodiments, X^{A} is C(R¹)₂, wherein each R¹ is optionally substituted C₁₋₆ aliphatic.

As defined above and described herein, Z^{A} is O, S, or NR. In some embodiments, Z^{A} is 0. In some embodiments, Z^{A} is S. In some embodiments, Z^{A} is NR.

In some embodiments, Ring A is:

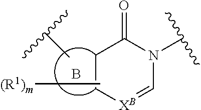

or a pharmaceutically acceptable salt thereof, wherein each R¹, Ring B, and m is as defined above and described herein both individually and in combination: and: XB is CR² or N.

As defined above and described herein, X^{B} is CR² or N. In some embodiments, X^{B} is CR². In some embodiments, X^{B} is N.

In some embodiments, Ring A is

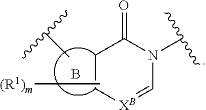

In some embodiments, Ring A is

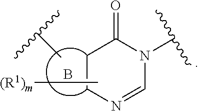

In some embodiments, Ring A is phenylenyl, naphthylenyl, pyridinylenyl,

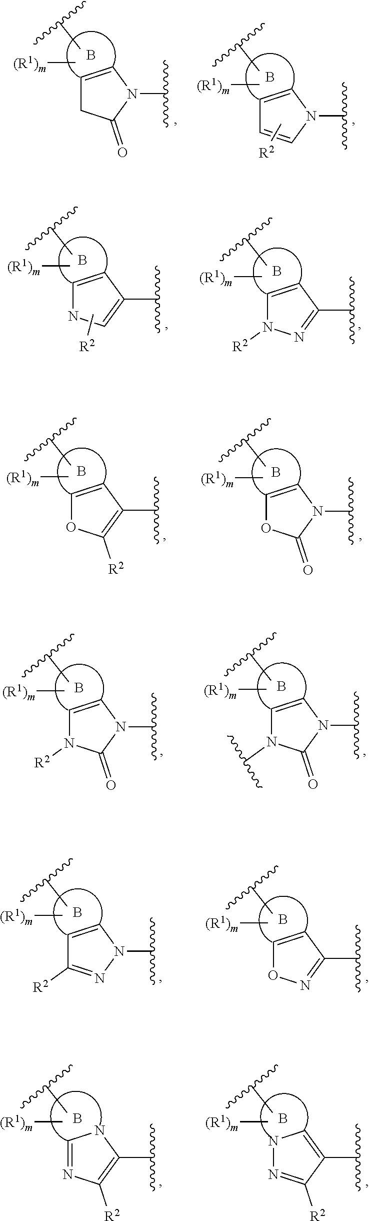

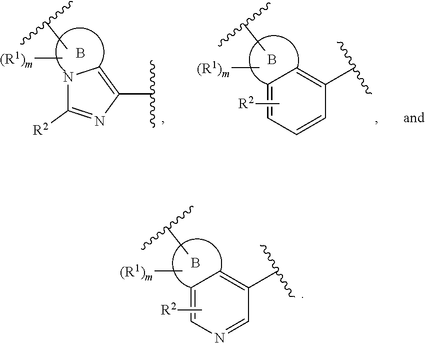

In some embodiments, Ring A is phenylenyl. In some embodiments, Ring A is naphthylenyl. In some embodiments, Ring A is pyridinylenyl. In some embodiments, Ring A is

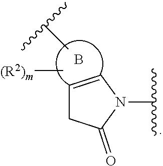

In some embodiments, Ring A is

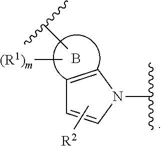

In some embodiments, Ring A is

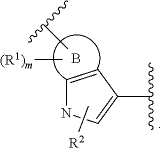

In some embodiments, Ring A is

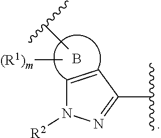

In some embodiments, Ring A is

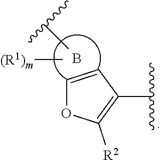

In some embodiments, Ring A is

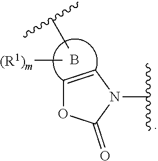

In some embodiments, Ring A is

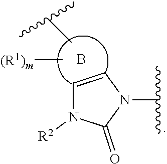

In some embodiments, Ring A is

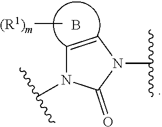

In some embodiments, Ring A is

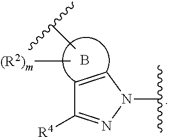

In some embodiments, Ring A is

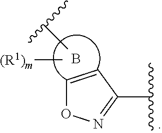

In some embodiments, Ring A is

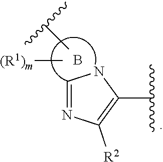

In some embodiments, Ring A is

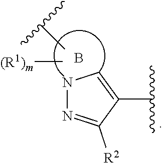

In some embodiments, Ring A is

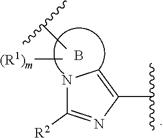

In some embodiments, Ring A is

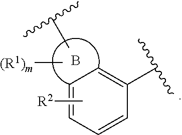

In some embodiments, Ring A is

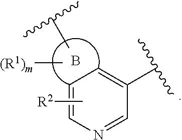

In some embodiments, Ring A is

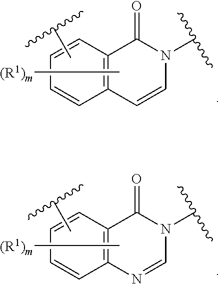

In some embodiments, Ring A is

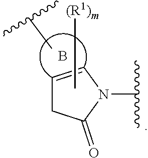

In some embodiments, Ring A is

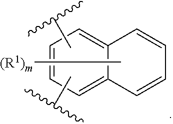

In some embodiments, Ring A is

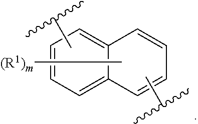

In some embodiments, Ring A is

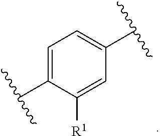

In some embodiments, Ring A is

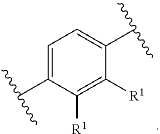

In some embodiments, Ring A is

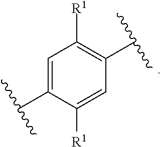

In some embodiments, Ring A is

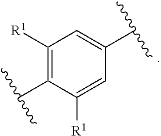

In some embodiments, Ring A is

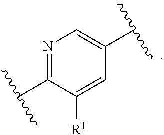

In some embodiments, Ring A is

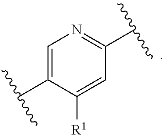

In some embodiments, Ring A is

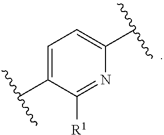

In some embodiments, Ring A is

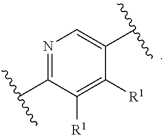

In some embodiments, Ring A is

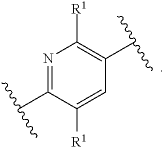

In some embodiments, Ring A is

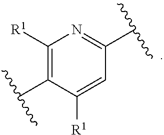

In some embodiments, Ring A is

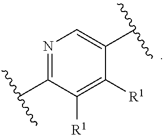

In some embodiments, Ring A is

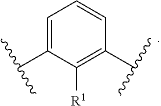

In some embodiments, Ring A is

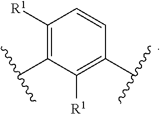

In some embodiments, Ring A is

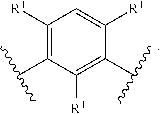

In some embodiments, Ring A is

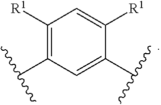

In some embodiments, Ring A is

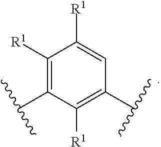

In some embodiments, Ring A is

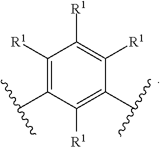

In some embodiments, Ring A is

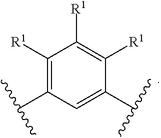

In some embodiments, Ring A is

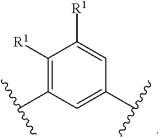

In some embodiments, Ring A is

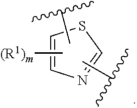

In some embodiments, Ring A is

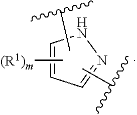

In some embodiments, Ring A is

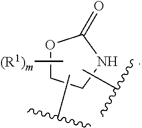

In some embodiments, Ring A is

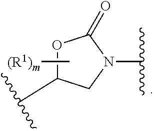

In some embodiments, Ring A is

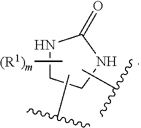

In some embodiments, Ring A is

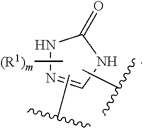

In some embodiments, Ring A is

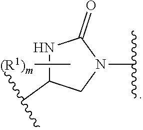

In some embodiments, Ring A is

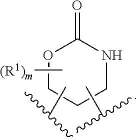

In some embodiments, Ring A is

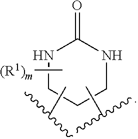

In some embodiments, Ring A is

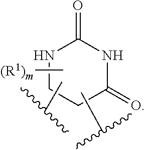

In some embodiments, Ring A is

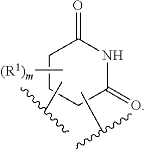

In some embodiments, Ring A is

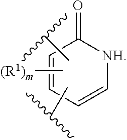

In some embodiments, Ring A is

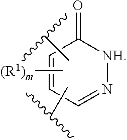

In some embodiments, Ring A is

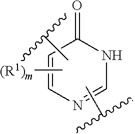

In some embodiments, Ring A is

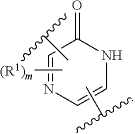

In some embodiments, Ring A is

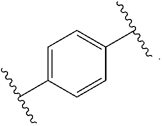 [SMILES: *c1ccc(*)cc1]

In other embodiments Ring A is a 6-10 membered monocyclic or bicyclic heteroaryl containing 1-4 heteroatoms independently selected from nitrogen, oxygen, and sulfur. In other embodiments Ring A is a 9-membered bicyclic heteroaryl containing 1-4 heteroatoms independently selected from nitrogen, oxygen, and sulfur. In some embodiments, Ring A is a 5,6-fused bicyclic heteroarylenyl containing 1-4 heteroatoms independently selected from nitrogen, oxygen, and sulfur.

In other embodiments Ring A is a 9-membered bicyclic heteroaryl containing 1 nitrogen and 1 oxygen heteroatom. In some embodiments, Ring A is a 5,6-fused bicyclic heteroarylenyl containing 1 nitrogen and 1 oxygen heteroatom. In some embodiments, Ring A is benzo[d]oxazolyl, benzo[d]thiazolyl, benzofuran, or benzo[b]thiophenyl. In some embodiments, Ring A is benzo[d]oxazolyl. In some embodiments, Ring A is benzo[d]thiazolyl. In some embodiments, Ring A is benzofuran. In some embodiments, Ring A is benzo[b]thiophenyl.

In other embodiments Ring A is a 9-membered bicyclic heteroaryl containing 1-3 nitrogen heteroatoms. In some embodiments, Ring A is a 5,6-fused bicyclic heteroarylenyl containing 1-3 nitrogen heteroatoms. In some embodiments, Ring A is indolyl, azaindolyl (e.g., 4-, 5-, 6-, or 7-azaindolyl), indazolyl, or azaindazolyl (e.g., 4-, 5-, 6-, or 7-azaindazolyl). In some embodiments, Ring A is indolyl. In some embodiments, Ring A is azaindolyl (e.g., 4-, 5-, 6-, or 7-azaindolyl). In some embodiments, Ring A is indazolyl. In some embodiments, Ring A is azaindazolyl (e.g., 4-, 5-, 6-, or 7-azaindazolyl).

In other embodiments Ring A is a 10-membered bicyclic heteroaryl containing 1-4 heteroatoms independently selected from nitrogen, oxygen, and sulfur. In some embodiments, Ring A is a 6,6-fused bicyclic heteroarylenyl containing 1-4 heteroatoms independently selected from nitrogen, oxygen, and sulfur. In some embodiments, Ring A is a 6,6-fused bicyclic heteroarylenyl containing 1 nitrogen heteroatom.

In some embodiments, Ring A is quinolinyl or isoquinolinyl. In some embodiments, Ring A is quinolinyl. In some embodiments, Ring A is isoquinolinyl.

In other embodiments Ring A is a 6-10 membered monocyclic or bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1 nitrogen heteroatom. In some embodiments, Ring A is a 5,6-fused bicyclic saturated or partially unsaturated heterocyclylenyl containing 1-4 heteroatoms independently selected from nitrogen, oxygen, and sulfur. In some embodiments, Ring A is a 5,6-fused bicyclic saturated or partially unsaturated heterocyclylenyl containing 1 nitrogen heteroatom.

In other embodiments Ring A is a 6-10 membered monocyclic or bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1 nitrogen heteroatom, and having an oxo group. In some embodiments, Ring A is a 5,6-fused bicyclic saturated or partially unsaturated heterocyclylenyl containing 1-4 heteroatoms independently selected from nitrogen, oxygen, and sulfur, and having an oxo group. In some embodiments, Ring A is a 5,6-fused bicyclic saturated or partially unsaturated heterocyclylenyl containing 1 nitrogen heteroatom, and having an oxo group.

In some embodiments, Ring A is indolinyl, indolinonyl, isoindolinyl, isoindolinonyl, isoindolinedionyl, pyrrolopyridinyl, or pyrrolopyrimidinyl. In some embodiments, Ring A is indolinyl. In some embodiments, Ring A is indolinonyl. In some embodiments, Ring A is isoindolinyl. In some embodiments, Ring A is isoindolinonyl. In some embodiments, Ring A is isoindolinedionyl. In some embodiments, Ring A is pyrrolopyridinyl. In some embodiments, Ring A is pyrrolopyrimidinyl.

In some embodiments, Ring A is benzoxazolyl, benzothiazolyl, indazolyl, or azaindazolyl. In some embodiments, Ring A is benzoxazolyl. In some embodiments, Ring A is benzothiazolyl. In some embodiments, Ring A is indazolyl. In some embodiments, Ring A is azaindazolyl (e.g., 4-, 5-, 6-, or 7-azaindazolyl).

In some embodiments, Ring A is 2,3-dihydrobenzofuranyl, indolinyl, or 2,3-dihydrobenzothiophenyl. In some embodiments, Ring A is 2,3-dihydrobenzofuranyl. In some embodiments, Ring A is indolinyl. In some embodiments, Ring A is 2,3-dihydrobenzothiophenyl.

In some embodiments, Ring A is

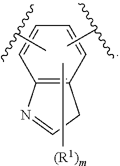

In some embodiments, Ring A is

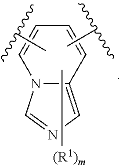

In some embodiments, Ring A is

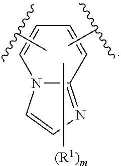

In some embodiments, Ring A is

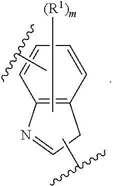

In some embodiments, Ring A is

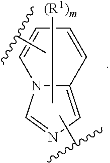

In some embodiments, Ring A is

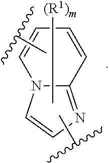

In some embodiments, Ring A is

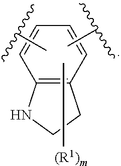

In some embodiments, Ring A is

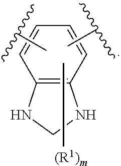

In some embodiments, Ring A is

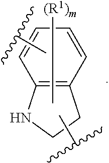

In some embodiments, Ring A is

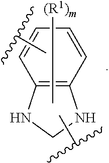

In some embodiments, Ring A is

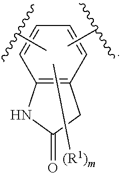

In some embodiments, Ring A is

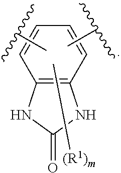

In some embodiments, Ring A is

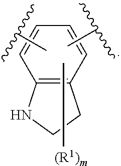

In some embodiments, Ring A is

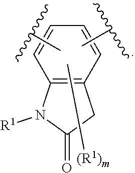

In some embodiments, Ring A is

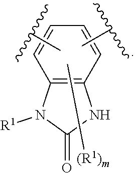

In some embodiments, Ring A is

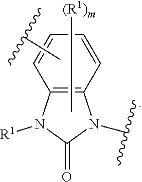

In some embodiments, Ring A is

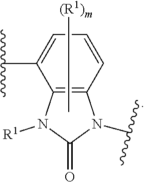

In some embodiments, Ring A is

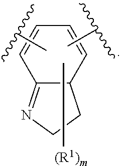

In some embodiments, Ring A is

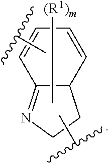

In some embodiments, Ring A is

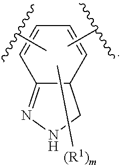

In some embodiments, Ring A is

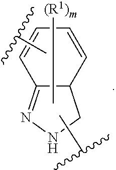

In some embodiments, Ring A is

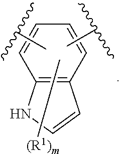

In some embodiments, Ring A is

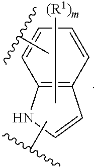

In some embodiments, Ring A is

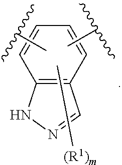

In some embodiments, Ring A is

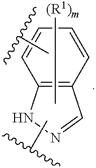

In some embodiments, Ring A is

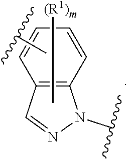

In some embodiments, Ring A is

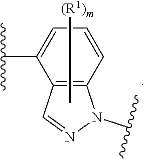

In some embodiments, Ring A is

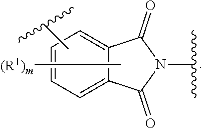

In some embodiments, Ring A is

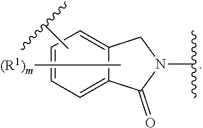

In some embodiments, Ring A is

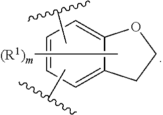

In some embodiments, Ring A is

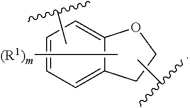

In some embodiments, Ring A is

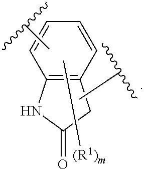

In some embodiments, Ring A is an 10-15 membered saturated or partially unsaturated tricyclic heterocyclylenyl having 1-5 heteroatoms independently selected from nitrogen, oxygen, and sulfur.

In some embodiments, Ring A is a 12-membered saturated or partially unsaturated tricyclic heterocyclylenyl having 1-5 heteroatoms independently selected from nitrogen, oxygen, and sulfur. In some embodiments, Ring A is a 12-membered saturated or partially unsaturated tricyclic heterocyclylenyl having 1-5 nitrogen heteroatoms. In some embodiments, Ring A is a 12-membered saturated or partially unsaturated tricyclic heterocyclylenyl having 3 nitrogen heteroatoms.

In some embodiments, Ring A is a 5,6,5-fused saturated or partially unsaturated tricyclic heterocyclylenyl having 1-5 heteroatoms independently selected from nitrogen, oxygen, and sulfur. In some embodiments, Ring A is a 5,6,5-fused membered saturated or partially unsaturated tricyclic heterocyclylenyl having 1-5 nitrogen heteroatoms. In some embodiments, Ring A is a 5,6,5-fused membered saturated or partially unsaturated tricyclic heterocyclylenyl having 3 nitrogen heteroatoms.

In some embodiments, Ring A is a 5,5,6-fused saturated or partially unsaturated tricyclic heterocyclylenyl having 1-5 heteroatoms independently selected from nitrogen, oxygen, and sulfur. In some embodiments, Ring A is a 5,5,6-fused membered saturated or partially unsaturated tricyclic heterocyclylenyl having 1-5 nitrogen heteroatoms. In some embodiments, Ring A is a 5,5,6-fused membered saturated or partially unsaturated tricyclic heterocyclylenyl having 3 nitrogen heteroatoms.

In some embodiments, Ring A is a 13-membered saturated or partially unsaturated tricyclic heterocyclylenyl having 1-5 heteroatoms independently selected from nitrogen, oxygen, and sulfur. In some embodiments, Ring A is a 13-membered saturated or partially unsaturated tricyclic heterocyclylenyl having 1-5 nitrogen heteroatoms. In some embodiments, Ring A is a 13-membered saturated or partially unsaturated tricyclic heterocyclylenyl having 3 nitrogen heteroatoms.

In some embodiments, Ring A is a 5,6,6-fused saturated or partially unsaturated tricyclic heterocyclylenyl having 1-5 heteroatoms independently selected from nitrogen, oxygen, and sulfur. In some embodiments, Ring A is a 5,6,6-fused membered saturated or partially unsaturated tricyclic heterocyclylenyl having 1-5 nitrogen heteroatoms. In some embodiments, Ring A is a 5,6,6-fused membered saturated or partially unsaturated tricyclic heterocyclylenyl having 3 nitrogen heteroatoms.

In some embodiments, Ring A is a 6,5,6-fused saturated or partially unsaturated tricyclic heterocyclylenyl having 1-5 heteroatoms independently selected from nitrogen, oxygen, and sulfur. In some embodiments, Ring A is a 6,5,6-fused membered saturated or partially unsaturated tricyclic heterocyclylenyl having 1-5 nitrogen heteroatoms. In some embodiments, Ring A is a 6,5,6-fused membered saturated or partially unsaturated tricyclic heterocyclylenyl having 3 nitrogen heteroatoms.

In some embodiments, Ring A is a 14-membered saturated or partially unsaturated tricyclic heterocyclylenyl having 1-5 heteroatoms independently selected from nitrogen, oxygen, and sulfur.

In some embodiments, Ring A is or an 10-15 membered tricyclic heteroarylenyl having 1-5 heteroatoms independently selected from nitrogen, oxygen, and sulfur.

In some embodiments, Ring A is a 12-membered saturated or partially unsaturated tricyclic heteroarylenyl having 1-5 heteroatoms independently selected from nitrogen, oxygen, and sulfur. In some embodiments, Ring A is a 12-membered saturated or partially unsaturated tricyclic heteroarylenyl having 1-5 nitrogen heteroatoms. In some embodiments, Ring A is a 12-membered saturated or partially unsaturated tricyclic heteroarylenyl having 3 nitrogen heteroatoms.

In some embodiments, Ring A is a 5,6,5-fused saturated or partially unsaturated tricyclic heteroarylenyl having 1-5 heteroatoms independently selected from nitrogen, oxygen, and sulfur. In some embodiments, Ring A is a 5,6,5-fused membered saturated or partially unsaturated tricyclic heteroarylenyl having 1-5 nitrogen heteroatoms. In some embodiments, Ring A is a 5,6,5-fused membered saturated or partially unsaturated tricyclic heteroarylenyl having 3 nitrogen heteroatoms.

In some embodiments, Ring A is a 5,5,6-fused saturated or partially unsaturated tricyclic heteroarylenyl having 1-5 heteroatoms independently selected from nitrogen, oxygen, and sulfur. In some embodiments, Ring A is a 5,5,6-fused membered saturated or partially unsaturated tricyclic heteroarylenyl having 1-5 nitrogen heteroatoms. In some embodiments, Ring A is a 5,5,6-fused membered saturated or partially unsaturated tricyclic heteroarylenyl having 3 nitrogen heteroatoms.

In some embodiments, Ring A is a 13-membered saturated or partially unsaturated tricyclic heteroarylenyl having 1-5 heteroatoms independently selected from nitrogen, oxygen, and sulfur. In some embodiments, Ring A is a 13-membered saturated or partially unsaturated tricyclic heteroarylenyl having 1-5 nitrogen heteroatoms. In some embodiments, Ring A is a 13-membered saturated or partially unsaturated tricyclic heteroarylenyl having 3 nitrogen heteroatoms.

In some embodiments, Ring A is a 5,6,6-fused saturated or partially unsaturated tricyclic heteroarylenyl having 1-5 heteroatoms independently selected from nitrogen, oxygen, and sulfur. In some embodiments, Ring A is a 5,6,6-fused membered saturated or partially unsaturated tricyclic heteroarylenyl having 1-5 nitrogen heteroatoms. In some embodiments, Ring A is a 5,6,6-fused membered saturated or partially unsaturated tricyclic heteroarylenyl having 3 nitrogen heteroatoms.

In some embodiments, Ring A is a 6,5,6-fused saturated or partially unsaturated tricyclic heteroarylenyl having 1-5 heteroatoms independently selected from nitrogen, oxygen, and sulfur. In some embodiments, Ring A is a 6,5,6-fused membered saturated or partially unsaturated tricyclic heteroarylenyl having 1-5 nitrogen heteroatoms. In some embodiments, Ring A is a 6,5,6-fused membered saturated or partially unsaturated tricyclic heteroarylenyl having 3 nitrogen heteroatoms.

In some embodiments, Ring A is a 14-membered saturated or partially unsaturated tricyclic heteroarylenyl having 1-5 heteroatoms independently selected from nitrogen, oxygen, and sulfur.

In some embodiments, Ring A is of formula ABC-1 or ABC-2:

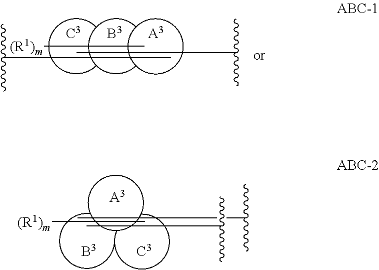

or a pharmaceutically acceptable salt thereof, wherein each R¹ and m is as defined above and described herein both individually and in combination: and:
each of Ring A³, Ring B³, and Ring C³ is independently a fused ring selected from a 3-7 membered saturated or partially unsaturated carbocyclylenyl: phenyl; a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

As defined above and described herein, each of Ring A³, Ring B³, and Ring C³ is independently a fused ring selected from a 3-7 membered saturated or partially unsaturated carbocyclylenyl; phenyl; a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

In some embodiments, Ring A³ is a fused 3-7 membered saturated or partially unsaturated carbocyclylenyl. In some embodiments, Ring A³ is a fused 4-7 membered saturated or partially unsaturated carbocyclylenyl. In some embodiments, Ring A³ is a fused 5-membered saturated or partially unsaturated carbocyclylenyl. In some embodiments, Ring A³ is a fused 6-membered saturated or partially unsaturated carbocyclylenyl. In some embodiments, Ring A³ is fused phenyl.

In some embodiments, Ring A³ is a fused 3-7 membered saturated or partially unsaturated heterocyclylenyl having 1-5 heteroatoms independently selected from nitrogen, oxygen, and sulfur. In some embodiments, Ring A³ is a fused 4-7 membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-2 nitrogen heteroatoms. In some embodiments, Ring A³ is a fused 5-membered saturated or partially unsaturated heterocyclylenyl having 1-5 heteroatoms independently selected from nitrogen, oxygen, and sulfur, and an oxo group. In some embodiments, Ring A³ is a fused 5-membered saturated or partially unsaturated heterocyclylenyl having 1-2 nitrogen heteroatoms, and an oxo group

In some embodiments, Ring A³ is a fused 6-membered saturated or partially unsaturated heterocyclylenyl having 1-5 heteroatoms independently selected from nitrogen, oxygen, and sulfur. In some embodiments, Ring A³ is a fused 6-membered saturated or partially unsaturated heterocyclylenyl having 1-2 nitrogen heteroatoms. In some embodiments, Ring A³ is a fused 6-membered saturated or partially unsaturated heterocyclylenyl having 1-5 heteroatoms independently selected from nitrogen, oxygen, and sulfur, and an oxo group. In some embodiments, Ring A³ is a fused 6-membered saturated or partially unsaturated heterocyclylenyl having 1-2 nitrogen heteroatoms, and an oxo group

In some embodiments, Ring A³ is a fused 5-6 membered heteroarylenyl having 1-5 heteroatoms independently selected from nitrogen, oxygen, and sulfur. In some embodiments, Ring A³ is a fused 5-membered heteroarylenyl having 1-5 heteroatoms independently selected from nitrogen, oxygen, and sulfur. In some embodiments, Ring A³ is a fused 6-membered heteroarylenyl having 1-5 heteroatoms independently selected from nitrogen, oxygen, and sulfur.

In some embodiments, Ring B³ is a fused 3-7 membered saturated or partially unsaturated carbocyclylenyl. In some embodiments, Ring B³ is a fused 4-7 membered saturated or partially unsaturated carbocyclylenyl. In some embodiments, Ring B³ is a fused 5-membered saturated or partially unsaturated carbocyclylenyl. In some embodiments, Ring B³ is a fused 6-membered saturated or partially unsaturated carbocyclylenyl. In some embodiments, Ring B³ is fused phenyl.

In some embodiments, Ring B³ is a fused 3-7 membered saturated or partially unsaturated heterocyclylenyl having 1-5 heteroatoms independently selected from nitrogen, oxygen, and sulfur. In some embodiments, Ring B³ is a fused 4-7 membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-2 nitrogen heteroatoms. In some embodiments, Ring B³ is a fused 5-membered saturated or partially unsaturated heterocyclylenyl having 1-5 heteroatoms independently selected from nitrogen, oxygen, and sulfur, and an oxo group. In some embodiments, Ring B³ is a fused 5-membered saturated or partially unsaturated heterocyclylenyl having 1-2 nitrogen heteroatoms, and an oxo group

In some embodiments, Ring B³ is a fused 6-membered saturated or partially unsaturated heterocyclylenyl having 1-5 heteroatoms independently selected from nitrogen, oxygen, and sulfur. In some embodiments, Ring B³ is a fused 6-membered saturated or partially unsaturated heterocyclylenyl having 1-2 nitrogen heteroatoms. In some embodiments, Ring B³ is a fused 6-membered saturated or partially unsaturated heterocyclylenyl having 1-5 heteroatoms independently selected from nitrogen, oxygen, and sulfur, and an oxo group. In some embodiments, Ring B³ is a fused 6-membered saturated or partially unsaturated heterocyclylenyl having 1-2 nitrogen heteroatoms, and an oxo group

In some embodiments, Ring B³ is a fused 5-6 membered heteroarylenyl having 1-5 heteroatoms independently selected from nitrogen, oxygen, and sulfur. In some embodiments, Ring B³ is a fused 5-membered heteroarylenyl having 1-5 heteroatoms independently selected from nitrogen, oxygen, and sulfur. In some embodiments, Ring B³ is a fused 6-membered heteroarylenyl having 1-5 heteroatoms independently selected from nitrogen, oxygen, and sulfur.

In some embodiments, Ring C³ is a fused 3-7 membered saturated or partially unsaturated carbocyclylenyl. In some embodiments, Ring C³ is a fused 4-7 membered saturated or partially unsaturated carbocyclylenyl. In some embodiments, Ring C³ is a fused 5-membered saturated or partially unsaturated carbocyclylenyl. In some embodiments, Ring C³ is a fused 6-membered saturated or partially unsaturated carbocyclylenyl. In some embodiments, Ring C³ is fused phenyl.

In some embodiments, Ring C³ is a fused 3-7 membered saturated or partially unsaturated heterocyclylenyl having 1-5 heteroatoms independently selected from nitrogen, oxygen, and sulfur. In some embodiments, Ring C³ is a fused 4-7 membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-2 nitrogen heteroatoms. In some embodiments, Ring C³ is a fused 5-membered saturated or partially unsaturated heterocyclylenyl having 1-5 heteroatoms independently selected from nitrogen, oxygen, and sulfur, and an oxo group. In some embodiments, Ring C³ is a fused 5-membered saturated or partially unsaturated heterocyclylenyl having 1-2 nitrogen heteroatoms, and an oxo group

In some embodiments, Ring C³ is a fused 6-membered saturated or partially unsaturated heterocyclylenyl having 1-5 heteroatoms independently selected from nitrogen, oxygen, and sulfur. In some embodiments, Ring C³ is a fused 6-membered saturated or partially unsaturated heterocyclylenyl having 1-2 nitrogen heteroatoms. In some embodiments, Ring C³ is a fused 6-membered saturated or partially unsaturated heterocyclylenyl having 1-5 heteroatoms independently selected from nitrogen, oxygen, and sulfur, and an oxo group. In some embodiments, Ring C³ is a fused 6-membered saturated or partially unsaturated heterocyclylenyl having 1-2 nitrogen heteroatoms, and an oxo group.

In some embodiments, Ring C³ is a fused 5-6 membered heteroarylenyl having 1-5 heteroatoms independently selected from nitrogen, oxygen, and sulfur. In some embodiments, Ring C³ is a fused 5-membered heteroarylenyl having 1-5 heteroatoms independently selected from nitrogen, oxygen, and sulfur. In some embodiments, Ring C³ is a fused 6-membered heteroarylenyl having 1-5 heteroatoms independently selected from nitrogen, oxygen, and sulfur.

In some embodiments, Ring A is of formula ABC-1 having a structure of formulae ABC-1a, ABC-1b, or ABC-1c:

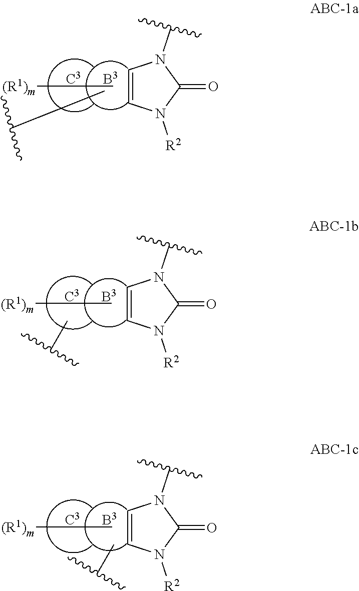

or a pharmaceutically acceptable salt thereof.

In some embodiments, Ring A is of formula ABC-1 having a structure of formulae ABC-1d, ABC-1e, or ABC-1f:

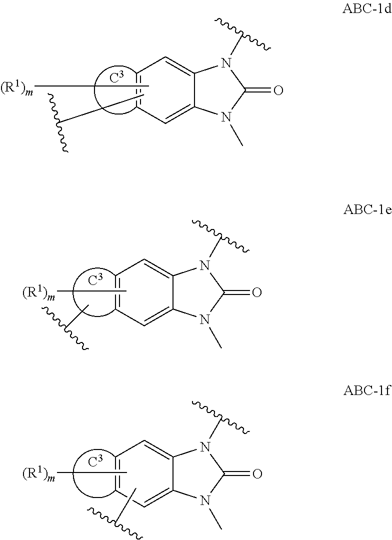

or a pharmaceutically acceptable salt thereof.

In some embodiments, Ring A is of formula ABC-1 having a structure of formulae ABC-1g, ABC-1h, ABC-1i, ABC-1j, ABC-1k, ABC-1l, ABC-1m, or ABC-1n:

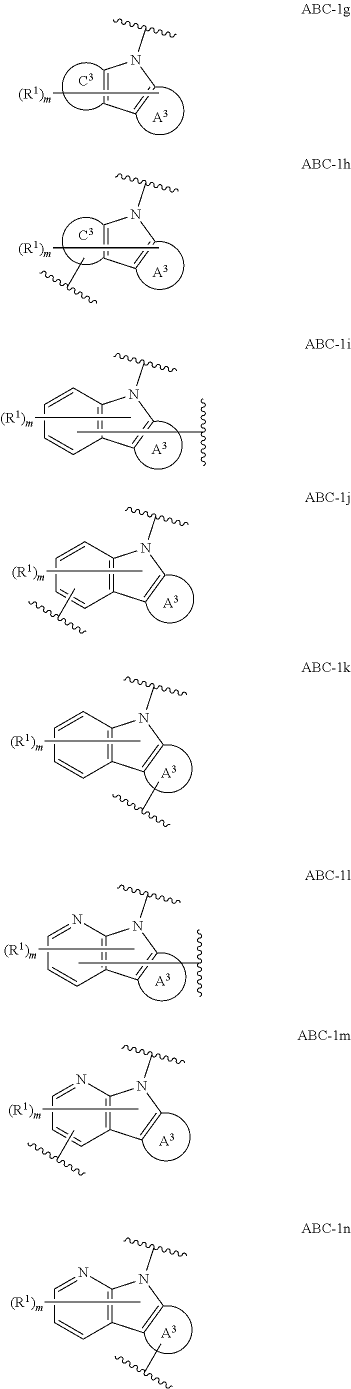

or a pharmaceutically acceptable salt thereof.

In some embodiments, Ring A is of formula ABC-2 having a structure of formulae ABC-2a, ABC-2b, ABC-2c, ABC-2d, ABC-2e, ABC-2f, or ABC-2g:

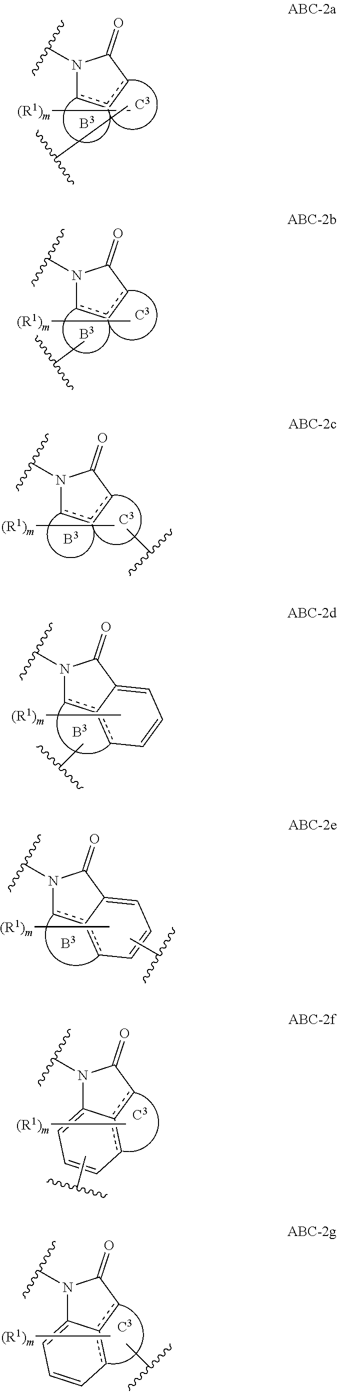

or a pharmaceutically acceptable salt thereof.

In some embodiments, Ring A is

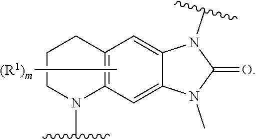

In some embodiments, Ring A is

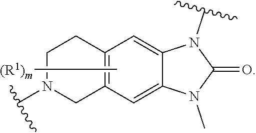

In some embodiments, Ring A is

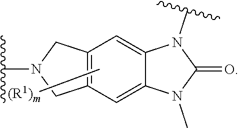

In some embodiments, Ring A is

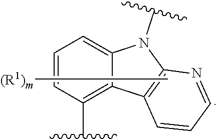

In some embodiments, Ring A is

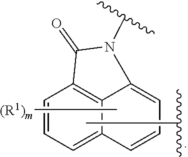

In some embodiments, Ring A is

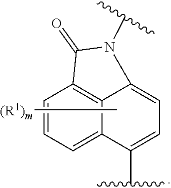

In some embodiments, Ring A and its R¹ substituents is

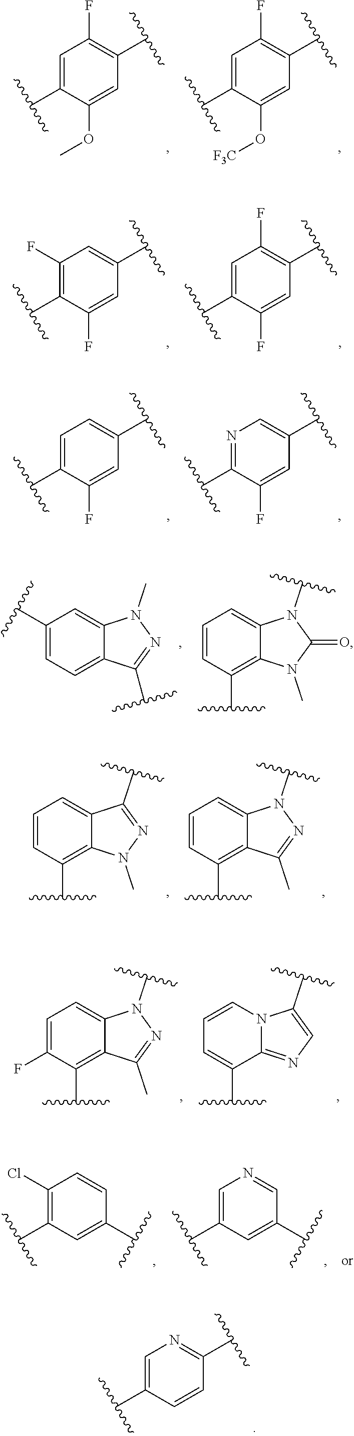

In some embodiments, Ring A, with its R¹ substituents and L¹ linker, is

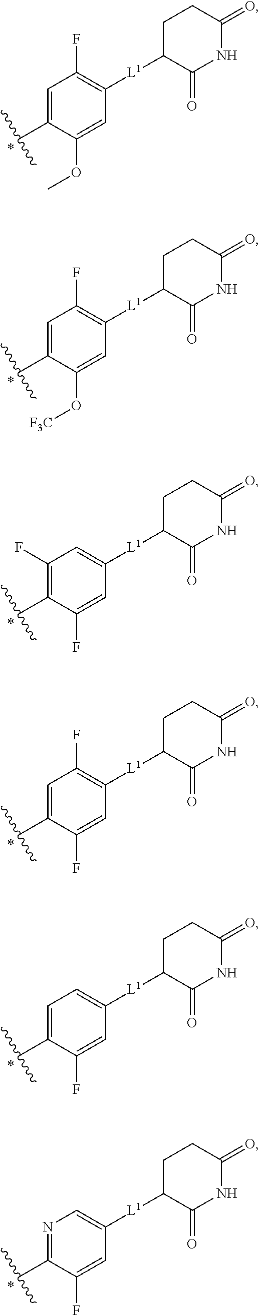

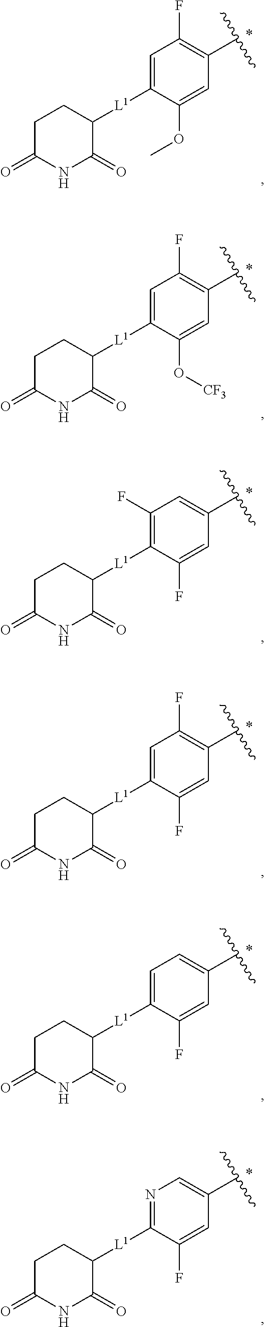

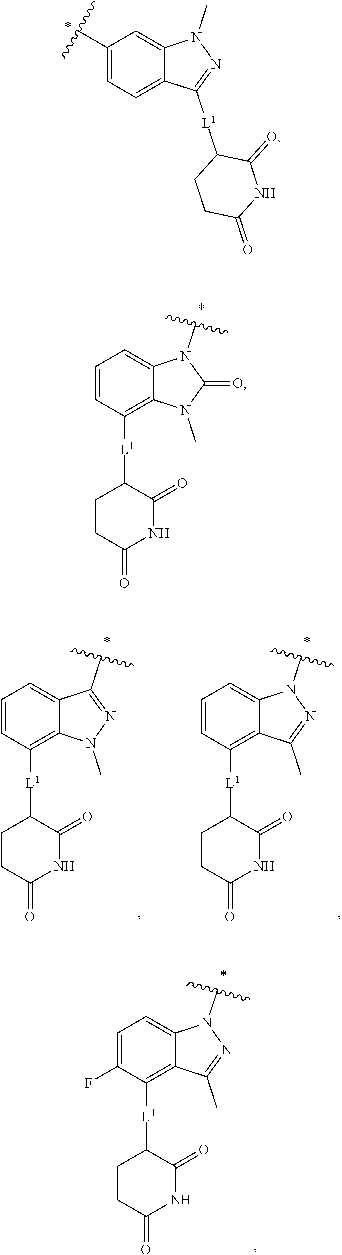

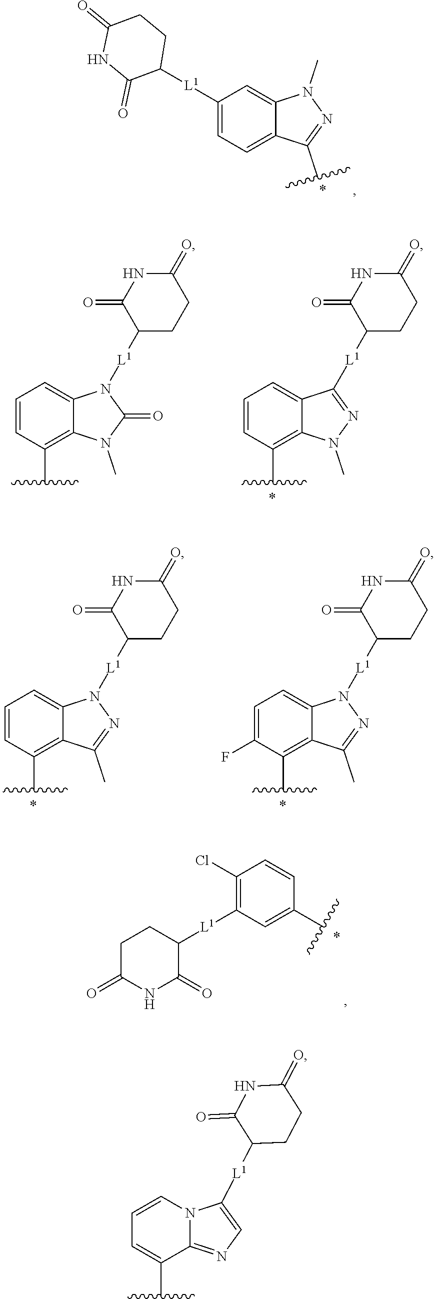

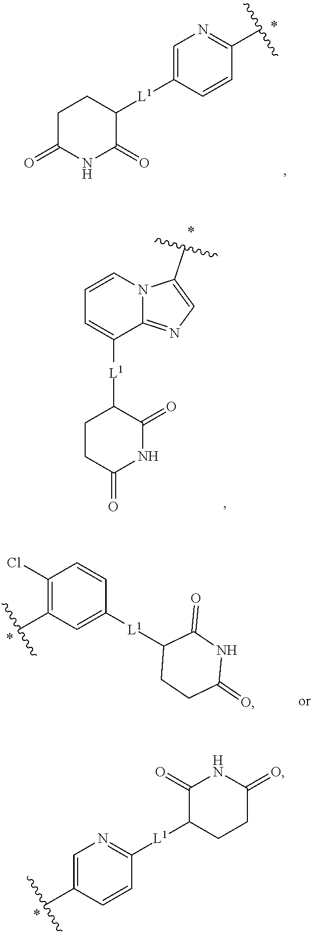

wherein

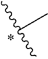

is the connection point to linker L.

In some embodiments, Ring A is selected from those depicted in the compounds of Table 1B below.

As defined above and described herein, Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur.

In some embodiments, Ring B is a fused ring selected from benzo or a 5-6 membered heteroaryl containing 1-4 heteroatoms independently selected from nitrogen, oxygen, and sulfur.

In some embodiments, Ring B is benzo. In some embodiments, Ring B is a 5-6 membered heteroaryl containing 1-4 heteroatoms independently selected from nitrogen, oxygen, and sulfur. In some embodiments, Ring B is pyridinyl.

In some embodiments, Ring B is a fused saturated or partially unsaturated 4-7 membered carbocyclyl. In some embodiments, Ring B is a fused saturated or partially unsaturated 4-6 membered carbocyclyl. In some embodiments, Ring B is a fused saturated or partially unsaturated 5-6 membered carbocyclyl. In some embodiments, Ring B is a saturated or partially unsaturated 4-7 membered heterocyclyl having 1-2 heteroatoms independently selected from nitrogen, oxygen, and sulfur. In some embodiments, Ring B is a saturated or partially unsaturated 4-6 membered heterocyclyl having 1-2 heteroatoms independently selected from nitrogen, oxygen, and sulfur. In some embodiments, Ring B is a saturated or partially unsaturated 5-6 membered heterocyclyl having 1-2 heteroatoms independently selected from nitrogen, oxygen, and sulfur.

In some embodiments, Ring B is selected from those depicted in the compounds of Table 1B below.

In some embodiments, Ring A and Ring B are

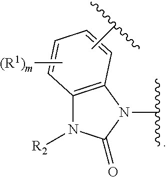

In some embodiments, Ring A and Ring B are

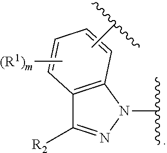

In some embodiments, Ring A and Ring B are

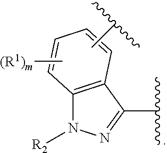

In some embodiments, Ring A and Ring B are

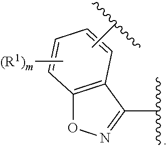

In some embodiments, Ring A and Ring B are

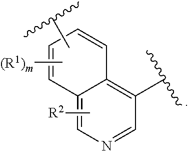

In some embodiments, LBM is

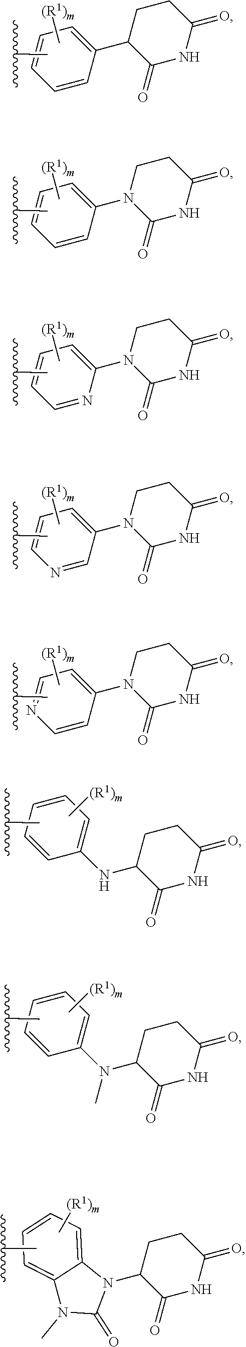

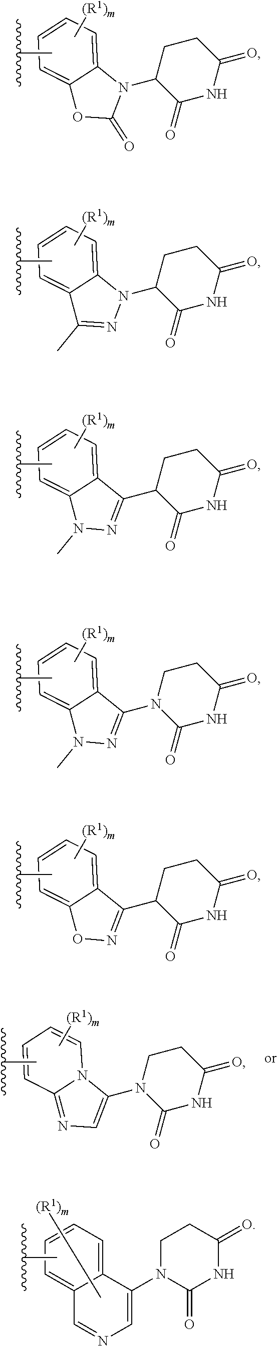

In some embodiments, LBM is

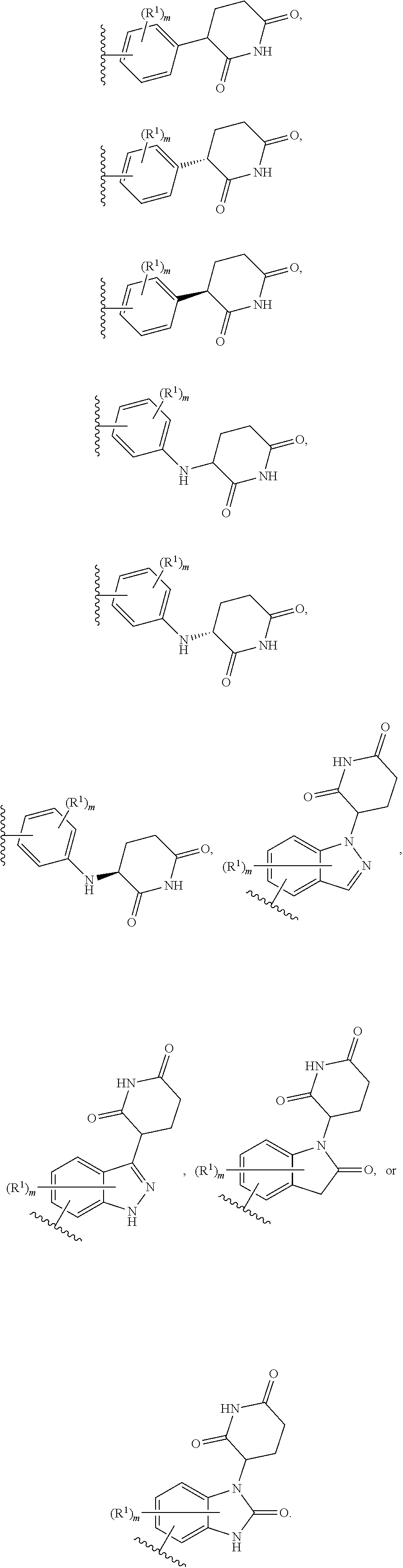

As defined above and described herein, R is independently hydrogen or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; or two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur; or two R groups on the same carbon or nitrogen are taken together with their intervening atoms to form an optionally substituted 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 3-7 membered saturated or partially unsaturated carbocyclic ring. In some embodiments, R is an optionally substituted 3-7 membered heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur. In some embodiments, two R groups on the same carbon or nitrogen are taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur. In some embodiments, two R groups on the same carbon or nitrogen are taken together with their intervening atoms to form an optionally substituted 4-11 membered saturated or partially unsaturated monocyclic, bicyclic, bridged bicyclic, or spirocyclic carbocyclic. In some embodiments, two R groups on the same carbon or nitrogen are optionally taken together with their intervening atoms to form an optionally substituted heterocyclic ring having 0-3 heteroatoms, in addition to the carbon or nitrogen from which the two R groups are attached, independently selected from nitrogen, oxygen, and sulfur.

In some embodiments, R is C₁₋₆ alkyl (e.g., methyl, ethyl, isopropyl, etc.). In some embodiments, R is C₁₋₆ haloalkyl (e.g., —CF₃, CHF₂, etc.).

In some embodiments, R is selected from those depicted in the compounds of Table 1B below.

As defined above and described herein, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0 or 1. In some embodiments, m is 1 or 2. In some embodiments, m is 0, 1, or 2.

In some embodiments, in is selected from those depicted in the compounds of Table 1B below.

In some embodiments, LBM is a non-IMiD (immune modulatory drug), e.g., not thalidomide or a derivative thereof wherein Ring A is phthalimide.

In some embodiments, LBM is

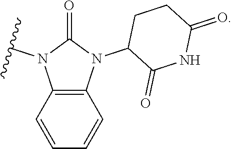 [SMILES: *n1c(=O)n(C2CCC(=O)NC2=O)c2ccccc21]

In some embodiments, LBM is

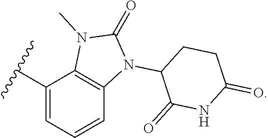 [SMILES: *c1cccc2c1n(C)c(=O)n2C1CCC(=O)NC1=O]

In some embodiments, LBM is

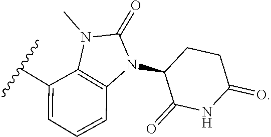 [SMILES: *c1cccc2c1n(C)c(=O)n2[C@H]1CCC(=O)NC1=O]

In some embodiments, LBM is

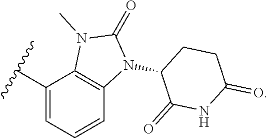 [SMILES: *c1cccc2c1n(C)c(=O)n2[C@@H]1CCC(=O)NC1=O]

In some embodiments, LBM is

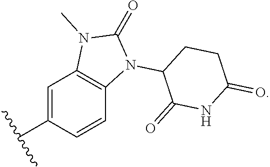 [SMILES: *c1ccc2c(c1)n(C)c(=O)n2C1CCC(=O)NC1=O]

In some embodiments, LBM is

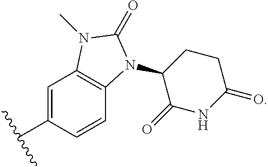 [SMILES: *c1ccc2c(c1)n(C)c(=O)n2[C@H]1CCC(=O)NC1=O]

In some embodiments, LBM is

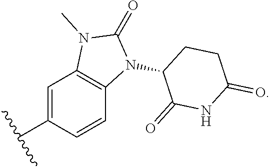 [SMILES: *c1ccc2c(c1)n(C)c(=O)n2[C@@H]1CCC(=O)NC1=O]

In some embodiments, LBM is

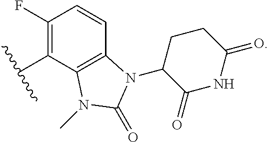 [SMILES: *c1c(F)ccc2c1n(C)c(=O)n2C1CCC(=O)NC1=O]

In some embodiments, LBM is

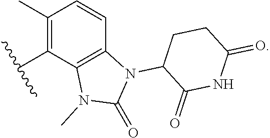 [SMILES: *c1c(C)ccc2c1n(C)c(=O)n2C1CCC(=O)NC1=O]

In some embodiments, LBM is

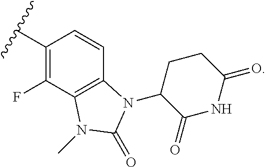 [SMILES: *c1ccc2c(c1F)n(C)c(=O)n2C1CCC(=O)NC1=O]

In some embodiments, LBM is

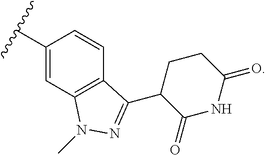 [SMILES: *c1ccc2c(C3CCC(=O)NC3=O)nn(C)c2c1]

In some embodiments, LBM is

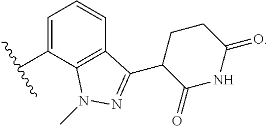 [SMILES: *c1cccc2c(C3CCC(=O)NC3=O)nn(C)c12]

In some embodiments, LBM is

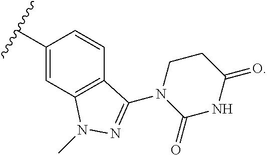 [SMILES: *c1ccc2c(N3CCC(=O)NC3=O)nn(C)c2c1]

In some embodiments, LBM is

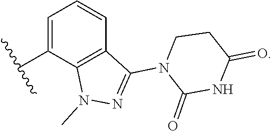 [SMILES: *c1cccc2c(N3CCC(=O)NC3=O)nn(C)c12]

In some embodiments, LBM is

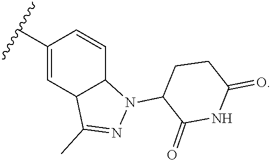 [SMILES: *C1=CC2C(C)=NN(C3CCC(=O)NC3=O)C2C=C1]

In some embodiments, LBM is

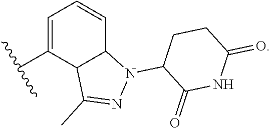 [SMILES: *C1=CC=CC2C1C(C)=NN2C1CCC(=O)NC1=O]

In some embodiments, LBM is

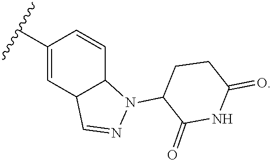 [SMILES: *C1=CC2C=NN(C3CCC(=O)NC3=O)C2C=C1]

In some embodiments, LBM is

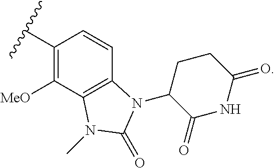 [SMILES: *c1ccc2c(c1OC)n(C)c(=O)n2C1CCC(=O)NC1=O]

In some embodiments, LBM is

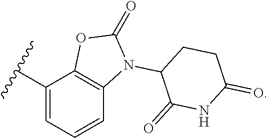 [SMILES: *c1cccc2c1oc(=O)n2C1CCC(=O)NC1=O]

In some embodiments, LBM is

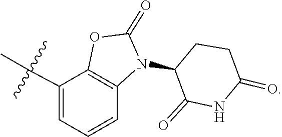 [SMILES: *c1cccc2c1oc(=O)n2[C@H]1CCC(=O)NC1=O]

In some embodiments, LBM is

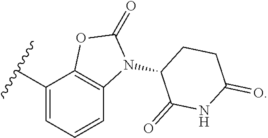 [SMILES: *c1cccc2c1oc(=O)n2[C@@H]1CCC(=O)NC1=O]

In some embodiments, LBM is

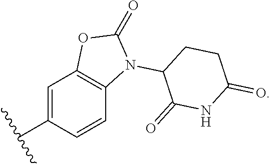 [SMILES: *c1ccc2c(c1)oc(=O)n2C1CCC(=O)NC1=O]

In some embodiments, LBM is

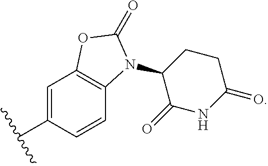 [SMILES: *c1ccc2c(c1)oc(=O)n2[C@H]1CCC(=O)NC1=O]

In some embodiments, LBM is

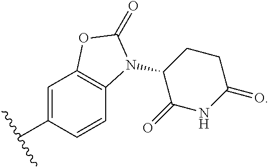 [SMILES: *c1ccc2c(c1)oc(=O)n2[C@@H]1CCC(=O)NC1=O]

In some embodiments, LBM is

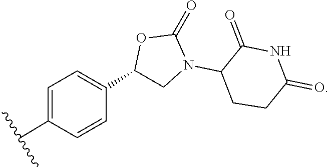 [SMILES: *c1ccc([C@H]2CN(C3CCC(=O)NC3=O)C(=O)O2)cc1]

In some embodiments, LBM is

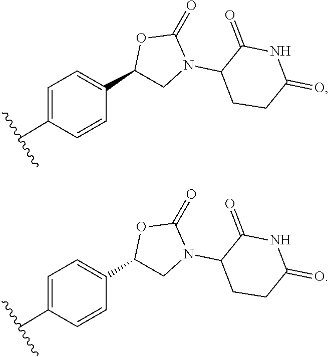 [SMILES: *c1ccc([C@@H]2CN(C3CCC(=O)NC3=O)C(=O)O2)cc1 ; *c1ccc([C@H]2CN(C3CCC(=O)NC3=O)C(=O)O2)cc1]

In some embodiments, LBM is

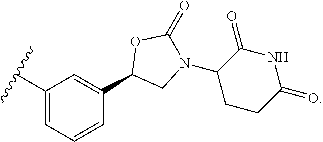 [SMILES: *c1cccc([C@@H]2CN(C3CCC(=O)NC3=O)C(=O)O2)c1]

In some embodiments, LBM is

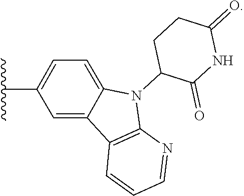 [SMILES: *c1ccc2c(c1)c1cccnc1n2C1CCC(=O)NC1=O]

In some embodiments, LBM is

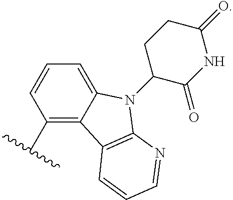 [SMILES: *c1cccc2c1c1cccnc1n2C1CCC(=O)NC1=O]

In some embodiments, LBM is

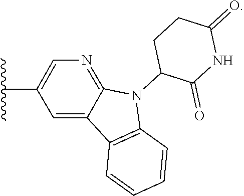 [SMILES: *c1cnc2c(c1)c1ccccc1n2C1CCC(=O)NC1=O]

In some embodiments, LBM is

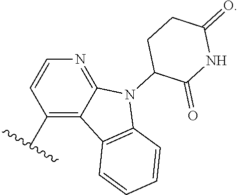 [SMILES: *c1ccnc2c1c1ccccc1n2C1CCC(=O)NC1=O]

In some embodiments, LBM is

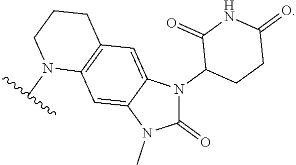 [SMILES: *N1CCCc2cc3c(cc21)n(C)c(=O)n3C1CCC(=O)NC1=O]

In some embodiments, LBM is

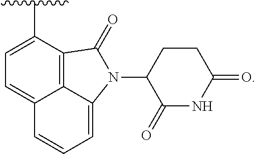 [SMILES: *c1ccc2cccc3c2c1C(=O)N3C1CCC(=O)NC1=O]

In some embodiments, LBM is

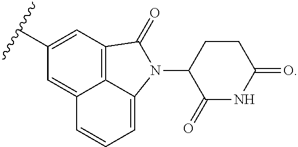 [SMILES: *c1cc2c3c(cccc3c1)N(C1CCC(=O)NC1=O)C2=O]

In some embodiments, LBM is

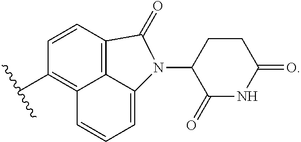 [SMILES: *c1ccc2c3c(cccc13)N(C1CCC(=O)NC1=O)C2=O]

In some embodiments, LBM is

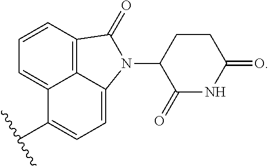 [SMILES: *c1ccc2c3c(cccc13)C(=O)N2C1CCC(=O)NC1=O]

In some embodiments, LBM is

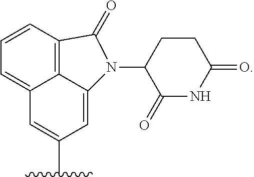 [SMILES: *c1cc2c3c(cccc3c1)C(=O)N2C1CCC(=O)NC1=O]

In some embodiments, LBM is

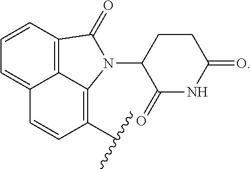 [SMILES: *c1ccc2cccc3c2c1N(C1CCC(=O)NC1=O)C3=O]

In some embodiments, LBM is

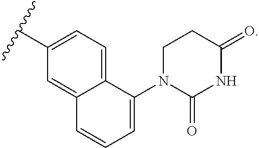 [SMILES: *c1ccc2c(N3CCC(=O)NC3=O)cccc2c1]

In some embodiments, LBM is

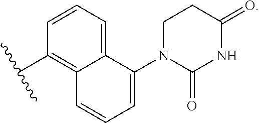 [SMILES: *c1cccc2c(N3CCC(=O)NC3=O)cccc12]

In some embodiments, LBM is

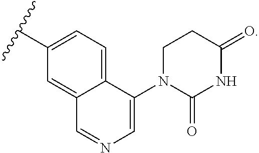 [SMILES: *c1ccc2c(N3CCC(=O)NC3=O)cncc2c1]

In some embodiments, LBM is

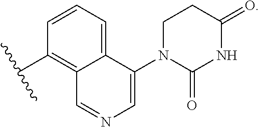 [SMILES: *c1cccc2c(N3CCC(=O)NC3=O)cncc12]

In some embodiments, LBM is

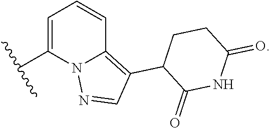 [SMILES: *c1cccc2c(C3CCC(=O)NC3=O)cnn12]

In some embodiments, LBM is

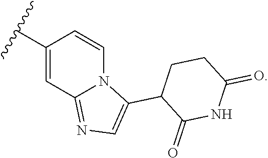 [SMILES: *c1ccn2c(C3CCC(=O)NC3=O)cnc2c1]

In some embodiments, LBM is

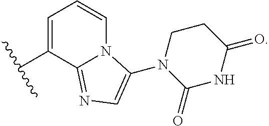 [SMILES: *c1cccn2c(N3CCC(=O)NC3=O)cnc12]

In some embodiments, LBM is

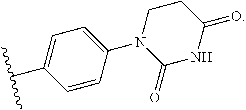 [SMILES: *c1ccc(N2CCC(=O)NC2=O)cc1]

In some embodiments, LBM is

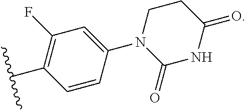 [SMILES: *c1ccc(N2CCC(=O)NC2=O)cc1F]

In some embodiments, LBM is

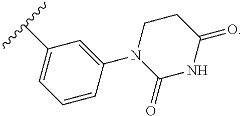 [SMILES: *c1cccc(N2CCC(=O)NC2=O)c1]

In some embodiments, LBM is

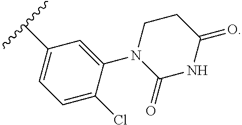 [SMILES: *c1ccc(Cl)c(N2CCC(=O)NC2=O)c1]

In some embodiments, LBM is

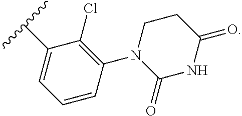 [SMILES: *c1cccc(N2CCC(=O)NC2=O)c1Cl]

In some embodiments, LBM is

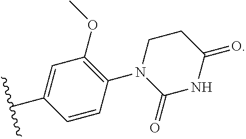 [SMILES: *c1ccc(N2CCC(=O)NC2=O)c(OC)c1]

In some embodiments, LBM is

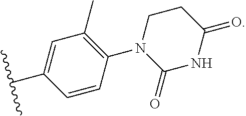 [SMILES: *c1ccc(N2CCC(=O)NC2=O)c(C)c1]

In some embodiments, LBM is

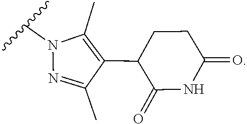 [SMILES: *n1nc(C)c(C2CCC(=O)NC2=O)c1C]

In some embodiments, LBM is

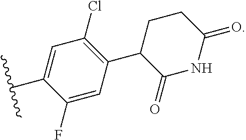 [SMILES: *c1cc(Cl)c(C2CCC(=O)NC2=O)cc1F]

In some embodiments, LBM is

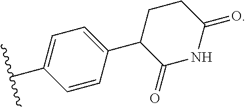 [SMILES: *c1ccc(C2CCC(=O)NC2=O)cc1]

In some embodiments, LBM is

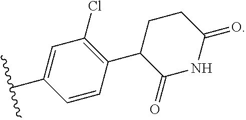 [SMILES: *c1ccc(C2CCC(=O)NC2=O)c(Cl)c1]

In some embodiments, LBM is

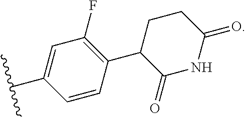 [SMILES: *c1ccc(C2CCC(=O)NC2=O)c(F)c1]

In some embodiments, LBM is

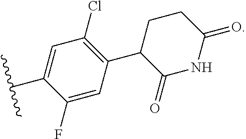 [SMILES: *c1cc(Cl)c(C2CCC(=O)NC2=O)cc1F]

In some embodiments, LBM is

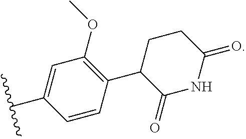 [SMILES: *c1ccc(C2CCC(=O)NC2=O)c(OC)c1]

In some embodiments, LBM is

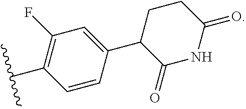 [SMILES: *c1ccc(C2CCC(=O)NC2=O)cc1F]

In some embodiments, LBM is

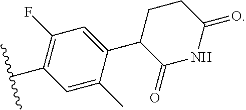 [SMILES: *c1cc(C)c(C2CCC(=O)NC2=O)cc1F]

In some embodiments, LBM is

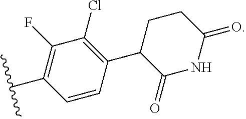 [SMILES: *c1ccc(C2CCC(=O)NC2=O)c(Cl)c1F]

In some embodiments, LBM is

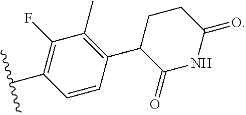 [SMILES: *c1ccc(C2CCC(=O)NC2=O)c(C)c1F]

In some embodiments, LBM is

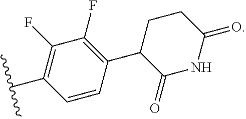 [SMILES: *c1ccc(C2CCC(=O)NC2=O)c(F)c1F]

In some embodiments, LBM is

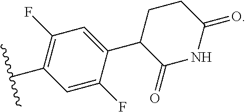 [SMILES: *c1cc(F)c(C2CCC(=O)NC2=O)cc1F]

In some embodiments, LBM is

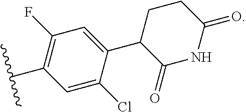 [SMILES: *c1cc(Cl)c(C2CCC(=O)NC2=O)cc1F]

In some embodiments, LBM is

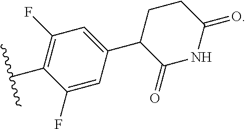 [SMILES: *c1c(F)cc(C2CCC(=O)NC2=O)cc1F]

In some embodiments, LBM is

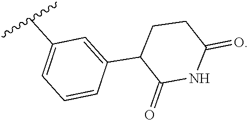 [SMILES: *c1cccc(C2CCC(=O)NC2=O)c1]

In some embodiments, LBM is

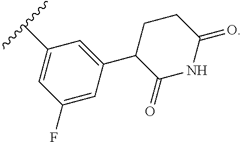 [SMILES: *c1cc(F)cc(C2CCC(=O)NC2=O)c1]

In some embodiments, LBM is

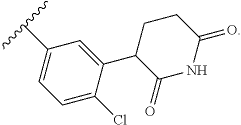 [SMILES: *c1ccc(Cl)c(C2CCC(=O)NC2=O)c1]

In some embodiments, LBM is

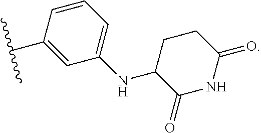 [SMILES: *c1cccc(NC2CCC(=O)NC2=O)c1]

In some embodiments, LBM is

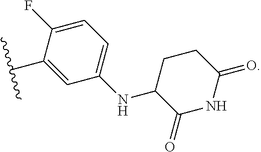 [SMILES: *c1cc(NC2CCC(=O)NC2=O)ccc1F]

In some embodiments, LBM is

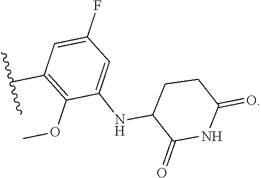 [SMILES: *c1cc(F)cc(NC2CCC(=O)NC2=O)c1OC]

In some embodiments, LBM is

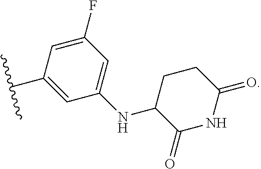 [SMILES: *c1cc(F)cc(NC2CCC(=O)NC2=O)c1]

In some embodiments, LBM is

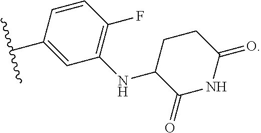 [SMILES: *c1ccc(F)c(NC2CCC(=O)NC2=O)c1]

In some embodiments, LBM is

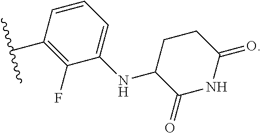 [SMILES: *c1cccc(NC2CCC(=O)NC2=O)c1F]

In some embodiments, LBM is

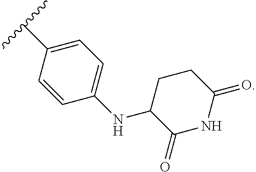 [SMILES: *c1ccc(NC2CCC(=O)NC2=O)cc1]

In some embodiments, LBM is

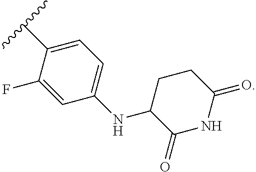 [SMILES: *c1ccc(NC2CCC(=O)NC2=O)cc1F]

In some embodiments, LBM is

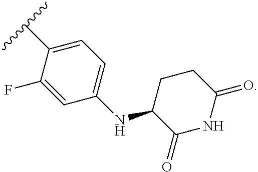 [SMILES: *c1ccc(N[C@H]2CCC(=O)NC2=O)cc1F]

In some embodiments, LBM is

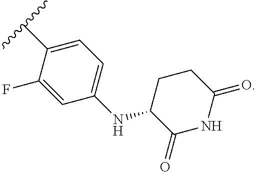 [SMILES: *c1ccc(N[C@@H]2CCC(=O)NC2=O)cc1F]

In some embodiments, LBM is

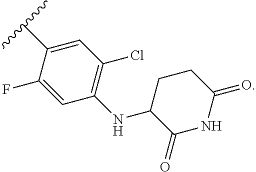 [SMILES: *c1cc(Cl)c(NC2CCC(=O)NC2=O)cc1F]

In some embodiments, LBM is

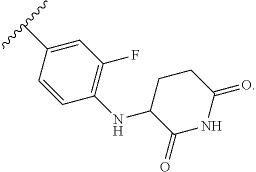 [SMILES: *c1ccc(NC2CCC(=O)NC2=O)c(F)c1]

In some embodiments, LBM is

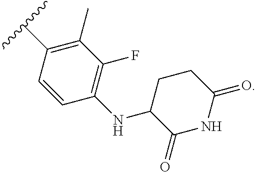 [SMILES: *c1ccc(NC2CCC(=O)NC2=O)c(F)c1C]

In some embodiments, LBM is

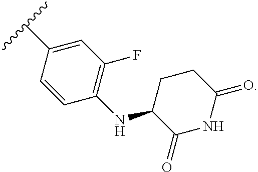 [SMILES: *c1ccc(N[C@H]2CCC(=O)NC2=O)c(F)c1]

In some embodiments, LBM is

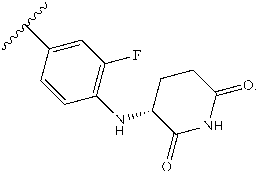 [SMILES: *c1ccc(N[C@@H]2CCC(=O)NC2=O)c(F)c1]

In some embodiments, LBM is

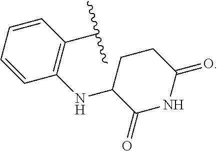 [SMILES: *c1ccccc1NC1CCC(=O)NC1=O]

In some embodiments, LBM is

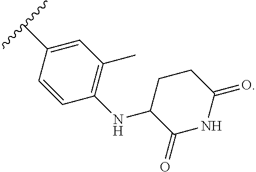 [SMILES: *c1ccc(NC2CCC(=O)NC2=O)c(C)c1]

In some embodiments, LBM is

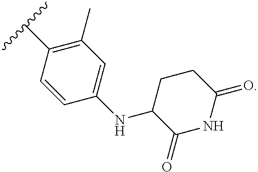 [SMILES: *c1ccc(NC2CCC(=O)NC2=O)cc1C]

In some embodiments, LBM is

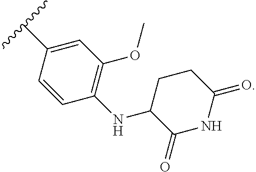 [SMILES: *c1ccc(NC2CCC(=O)NC2=O)c(OC)c1]

In some embodiments, LBM is

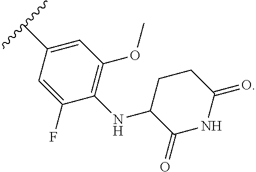 [SMILES: *c1cc(F)c(NC2CCC(=O)NC2=O)c(OC)c1]

In some embodiments, LBM is

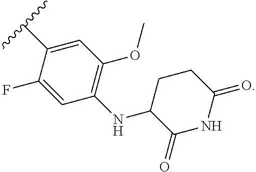 [SMILES: *c1cc(OC)c(NC2CCC(=O)NC2=O)cc1F]

In some embodiments, LBM is

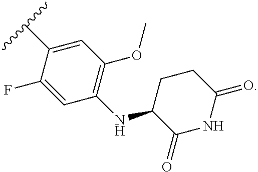 [SMILES: *c1cc(OC)c(N[C@H]2CCC(=O)NC2=O)cc1F]

In some embodiments, LBM is

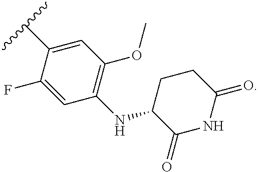 [SMILES: *c1cc(OC)c(N[C@@H]2CCC(=O)NC2=O)cc1F]

In some embodiments, LBM is

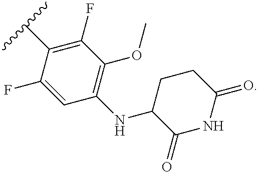 [SMILES: *c1c(F)cc(NC2CCC(=O)NC2=O)c(OC)c1F]

In some embodiments, LBM is

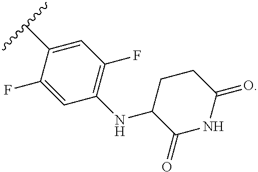 [SMILES: *c1cc(F)c(NC2CCC(=O)NC2=O)cc1F]

In some embodiments, LBM is

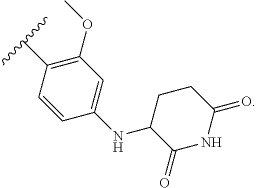 [SMILES: *c1ccc(NC2CCC(=O)NC2=O)cc1OC]

In some embodiments, LBM is

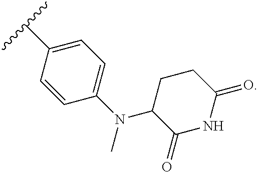 [SMILES: *c1ccc(N(C)C2CCC(=O)NC2=O)cc1]

In some embodiments, LBM is

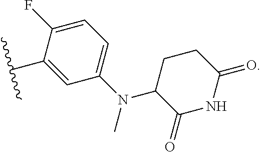 [SMILES: *c1cc(N(C)C2CCC(=O)NC2=O)ccc1F]

In some embodiments, LBM is

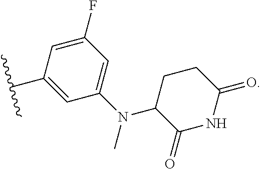 [SMILES: *c1cc(F)cc(N(C)C2CCC(=O)NC2=O)c1]

In some embodiments, LBM is

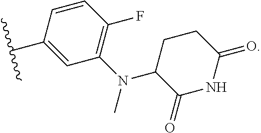 [SMILES: *c1ccc(F)c(N(C)C2CCC(=O)NC2=O)c1]

In some embodiments, LBM is

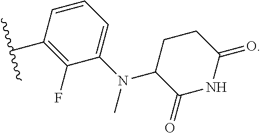 [SMILES: *c1cccc(N(C)C2CCC(=O)NC2=O)c1F]

In some embodiments, LBM is

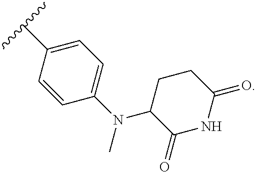 [SMILES: *c1ccc(N(C)C2CCC(=O)NC2=O)cc1]

In some embodiments, LBM is

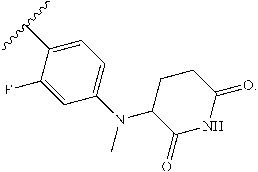 [SMILES: *c1ccc(N(C)C2CCC(=O)NC2=O)cc1F]

In some embodiments, LBM is

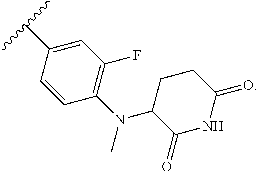 [SMILES: *c1ccc(N(C)C2CCC(=O)NC2=O)c(F)c1]

In some embodiments, LBM is

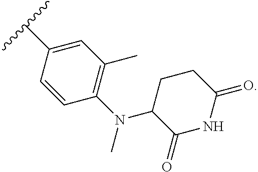 [SMILES: *c1ccc(N(C)C2CCC(=O)NC2=O)c(C)c1]

In some embodiments, LBM is

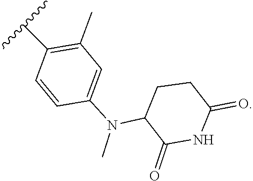 [SMILES: *c1ccc(N(C)C2CCC(=O)NC2=O)cc1C]

In some embodiments, LBM is

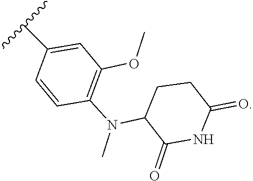 [SMILES: *c1ccc(N(C)C2CCC(=O)NC2=O)c(OC)c1]

In some embodiments, LBM is

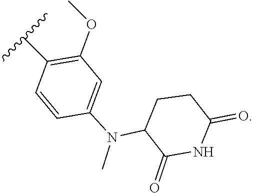 [SMILES: *c1ccc(N(C)C2CCC(=O)NC2=O)cc1OC]

In some embodiments, LBM is

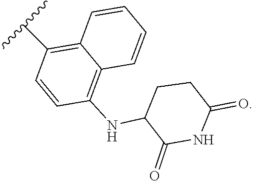 [SMILES: *c1ccc(NC2CCC(=O)NC2=O)c2ccccc12]

In some embodiments, LBM is

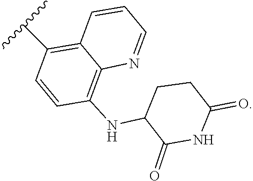 [SMILES: *c1ccc(NC2CCC(=O)NC2=O)c2ncccc12]

In some embodiments, LBM is

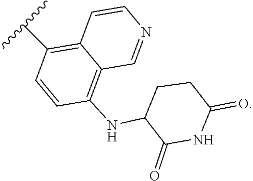 [SMILES: *c1ccc(NC2CCC(=O)NC2=O)c2cnccc12]

In some embodiments, LBM is

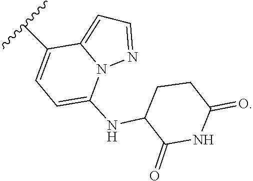 [SMILES: *c1ccc(NC2CCC(=O)NC2=O)n2nccc12]

In some embodiments, LBM is

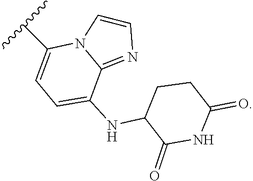 [SMILES: *c1ccc(NC2CCC(=O)NC2=O)c2nccn12]

In some embodiments, LBM is

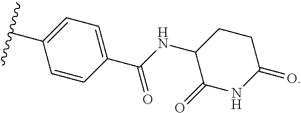 [SMILES: *c1ccc(C(=O)NC2CCC(=O)NC2=O)cc1]

In some embodiments, LBM is

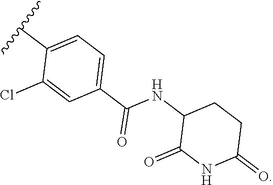 [SMILES: *c1ccc(C(=O)NC2CCC(=O)NC2=O)cc1Cl]

In some embodiments, LBM is

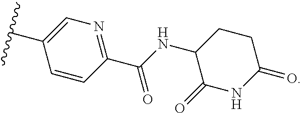 [SMILES: *c1ccc(C(=O)NC2CCC(=O)NC2=O)nc1]

In some embodiments, LBM is

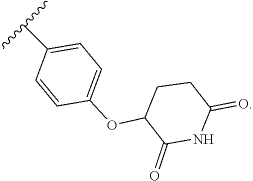 [SMILES: *c1ccc(OC2CCC(=O)NC2=O)cc1]

In some embodiments, LBM is

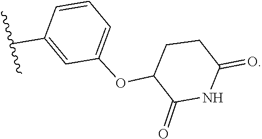 [SMILES: *c1cccc(OC2CCC(=O)NC2=O)c1]

In some embodiments, LBM is

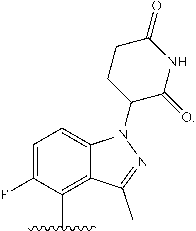 [SMILES: *c1c(F)ccc2c1c(C)nn2C1CCC(=O)NC1=O]

In some embodiments, LBM is

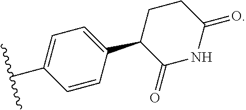 [SMILES: *c1ccc([C@H]2CCC(=O)NC2=O)cc1]

In some embodiments, LBM is

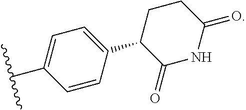 [SMILES: *c1ccc([C@@H]2CCC(=O)NC2=O)cc1]

In some embodiments, LBM is

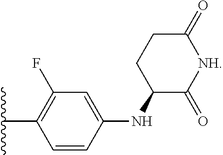 [SMILES: *c1ccc(N[C@H]2CCC(=O)NC2=O)cc1F]

In some embodiments, LBM is

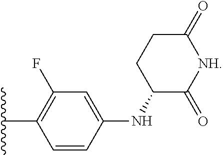 [SMILES: *c1ccc(N[C@@H]2CCC(=O)NC2=O)cc1F]

In certain embodiments, the present invention provides a compound of formula I-aa-1 as a compound of any one of the following formulae:

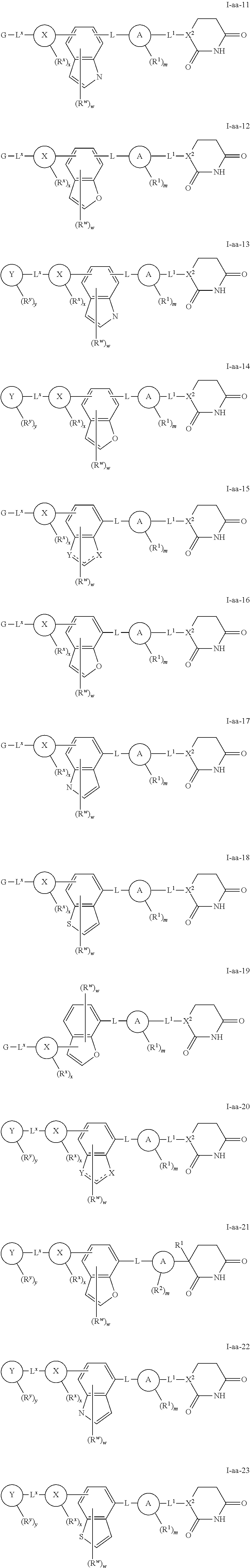

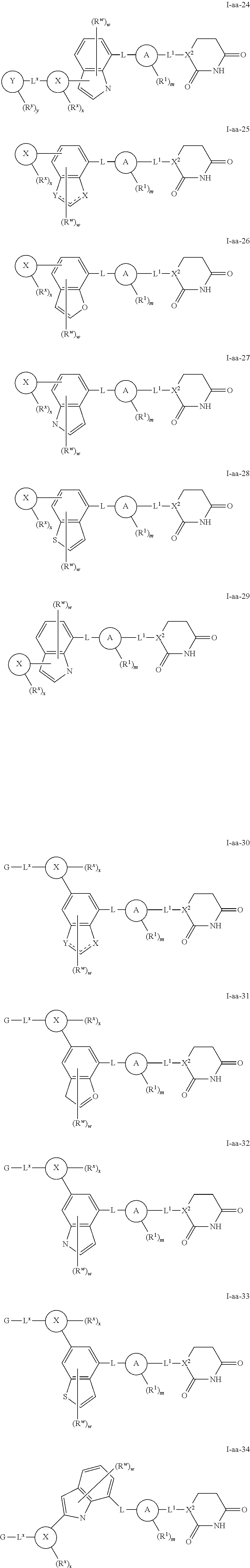

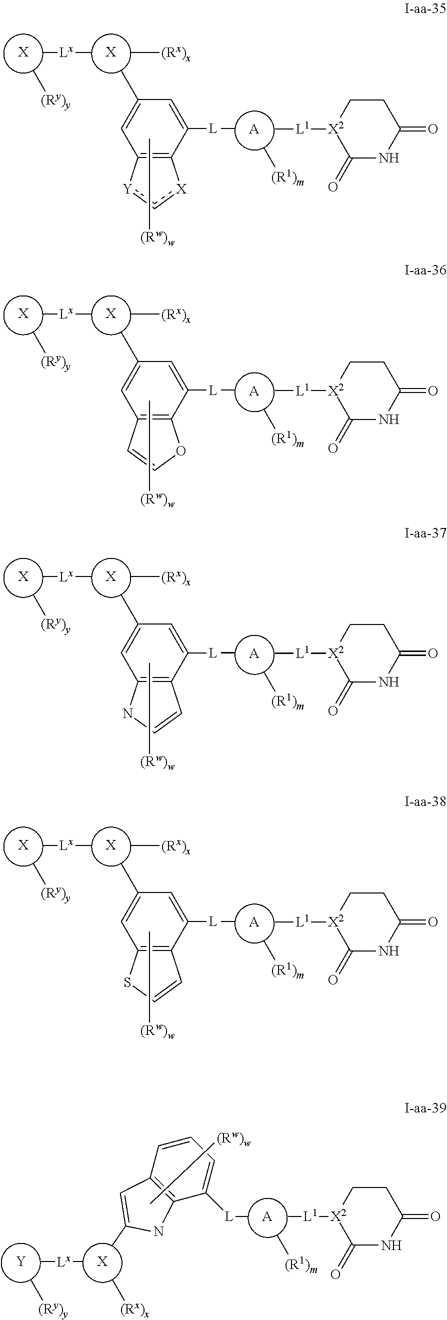

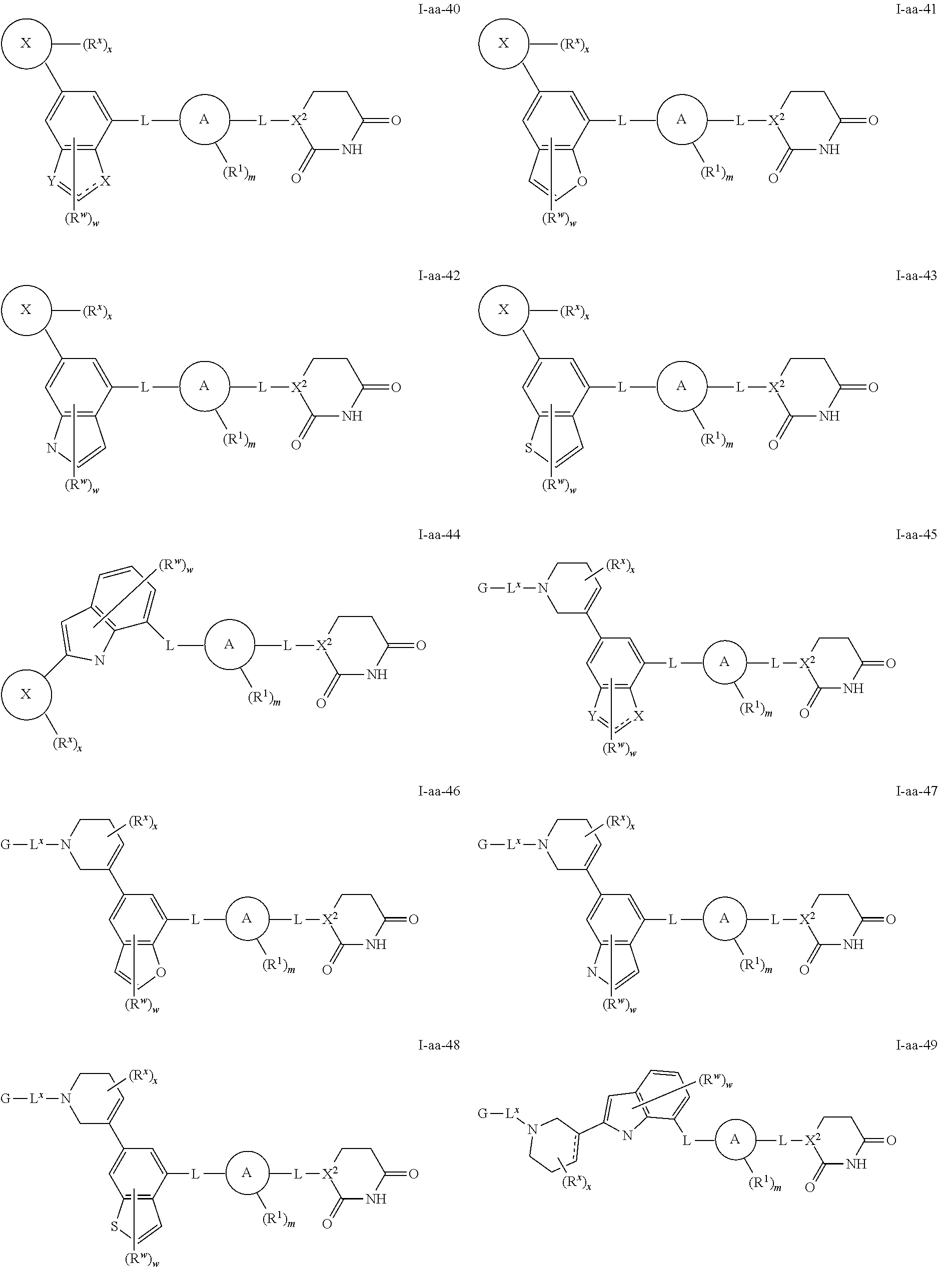

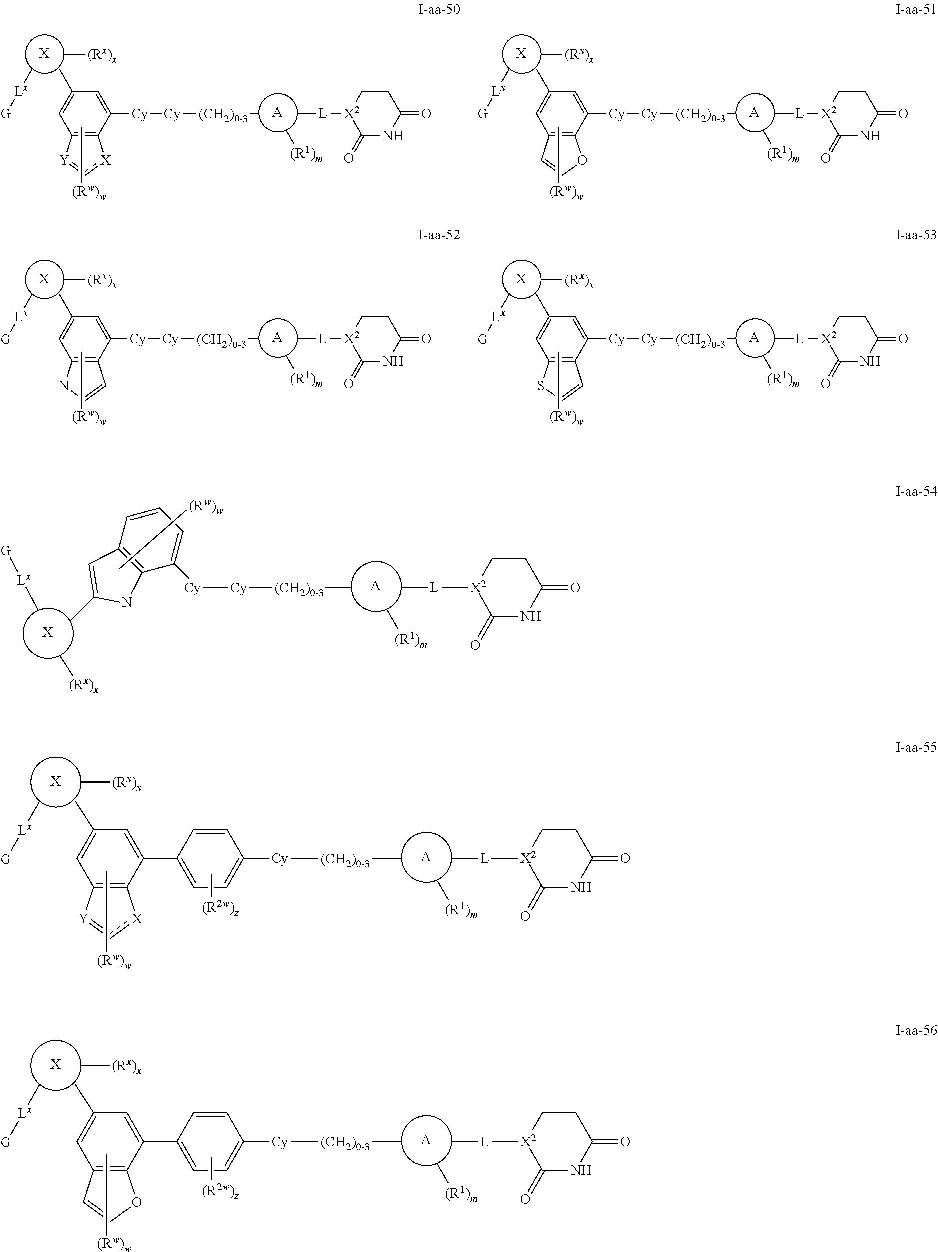

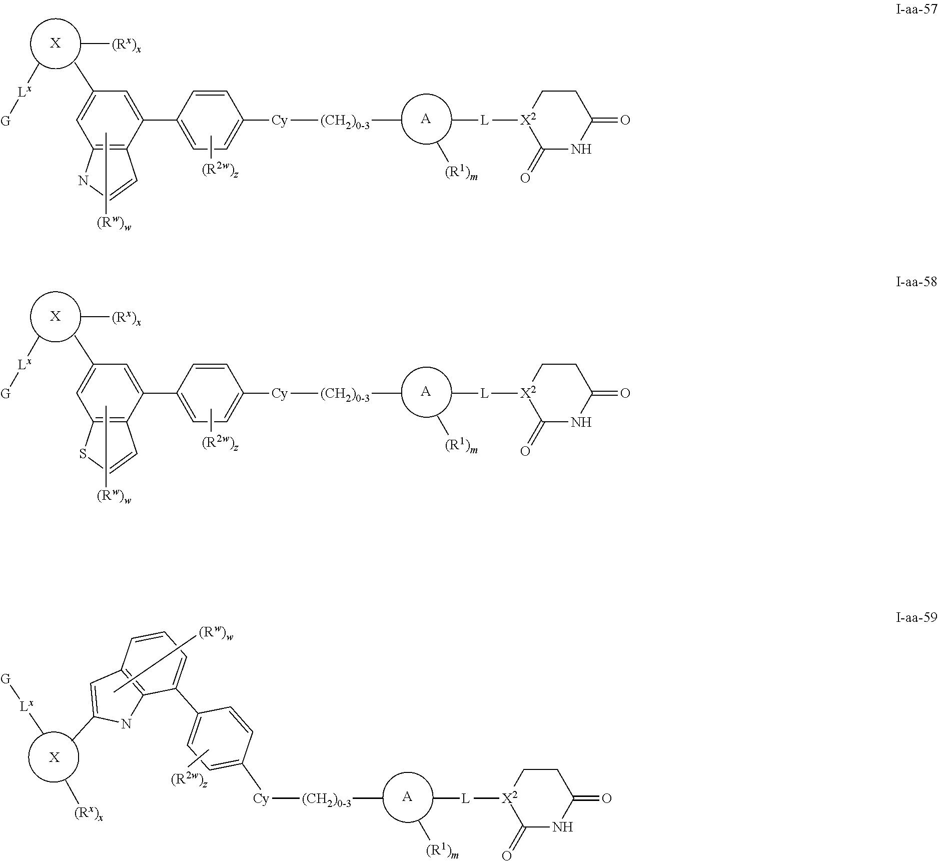

or a pharmaceutically acceptable salt thereof, wherein each variable is as defined above and described herein both individually and in combination.

In some embodiments, the present invention provides a compound of formula I, wherein LBM is an E3 ubiquitin ligase (cereblon) binding moiety thereby forming a compound of formula I-oo-1, I-oo-2, I-oo-3, I-oo-4, I-oo-5, I-oo-6, I-oo-7, I-oo-8, I-oo-9, or I-oo-10 respectively:

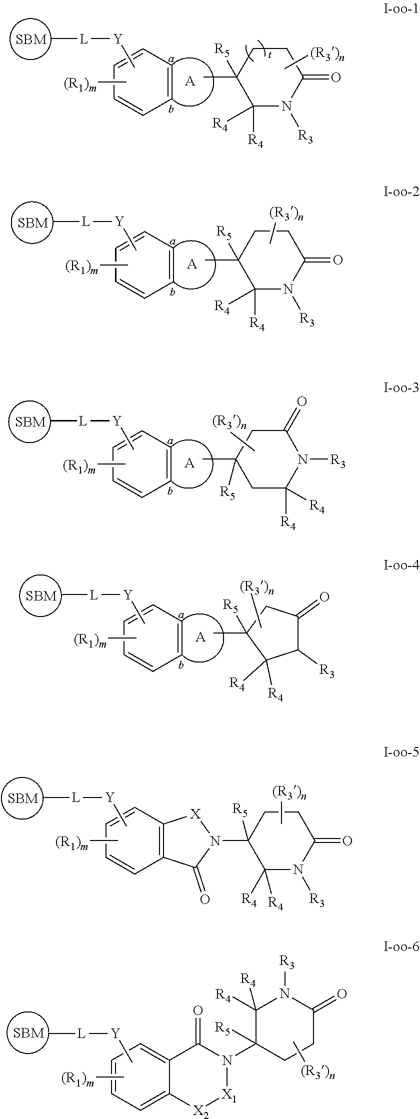

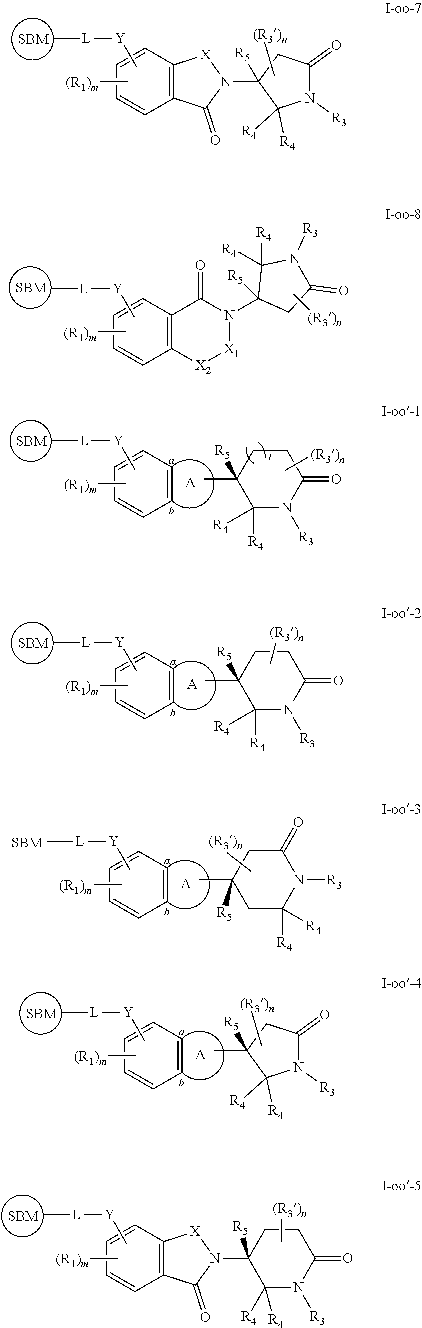

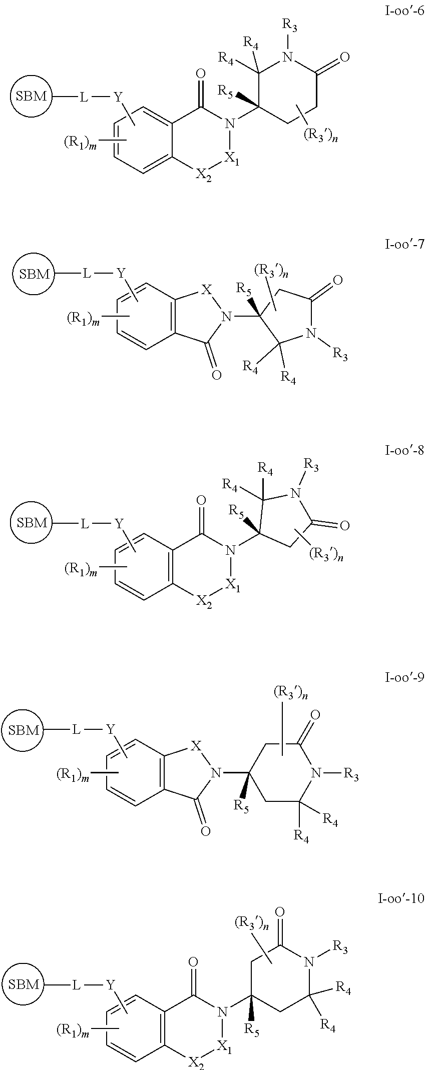

or a compound of formula I-oo′-1, I-oo′-2, I-oo′-3, I-oo′-4, I-oo′-5, I-oo′-6, I-oo′-7, I-oo′-8, I-oo′-9, or I-oo′-10 respectively:

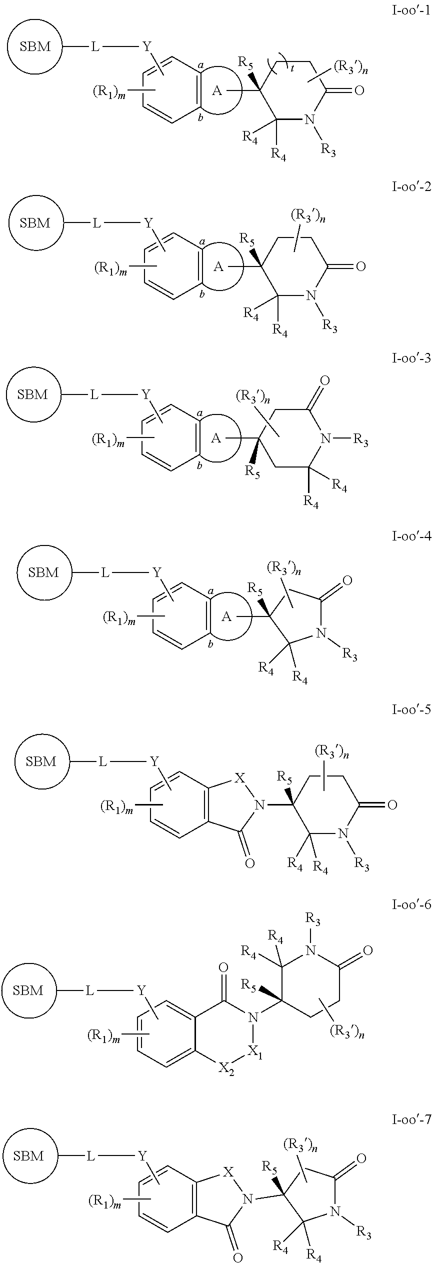

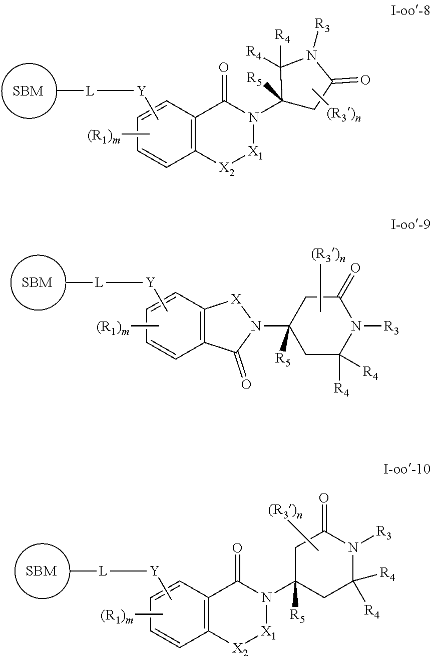

or a compound of formula I-oo″-1, I-oo″-2, I-oo″-3, I-oo″-4, I-oo″-5. I-oo″ ~-6, I-oo″-7, I-oo″-8, I-oo″-PG-87C 9, or I-oo″-10 respectively:

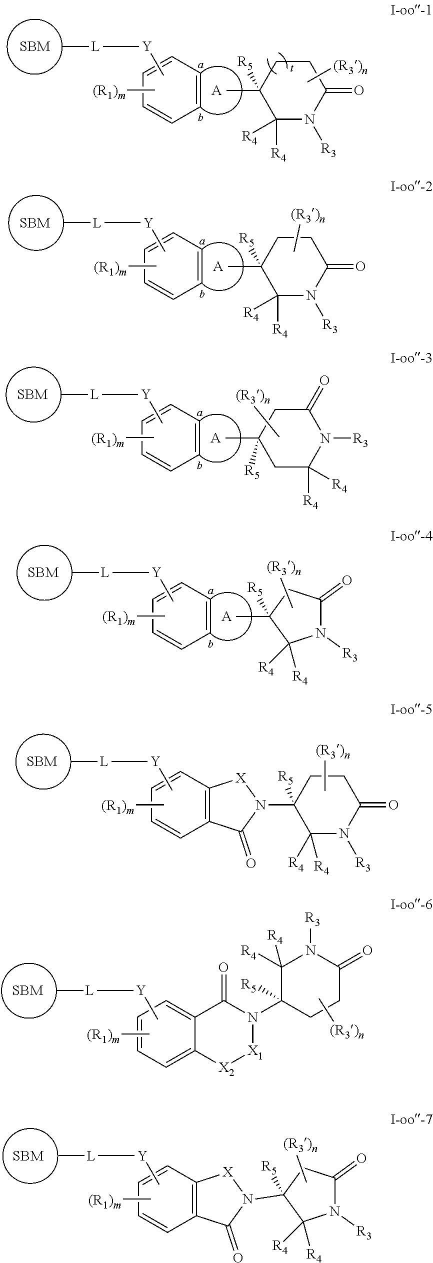

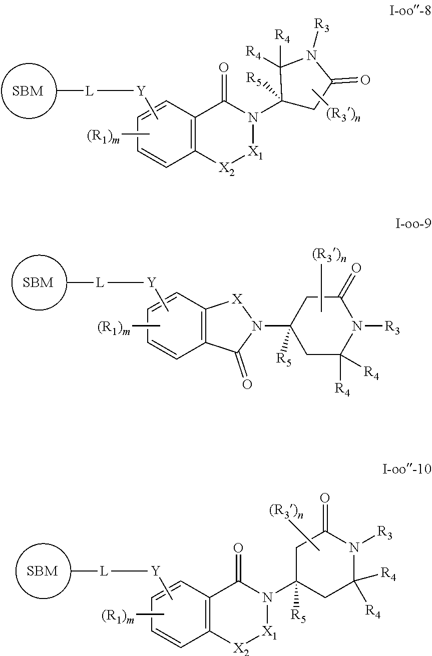

or a pharmaceutically acceptable salt thereof, wherein:

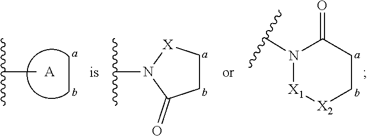

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁-X₂ is C(R₃)═N or C(R³)₂—C(R³)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₅ cycloalkyl, 3- to 8-membered heterocycloalkyl, C(O)—C₁-C₆ alkyl, C(O)—C₂-C₆ alkenyl, C(O)—C₃-C₅ cycloalkyl, or C(O)-3- to 8-membered heterocycloalkyl, and
R₂ is optionally substituted with one or more of halogen, N(Rₐ)₂, NHC(O)Rₐ, NHC(O)ORₐ, OR_{b}, C₃-C₅ cycloalkyl, 3- to 8-membered heterocycloalkyl, C₆-C₁₀ aryl, or 5- to 10-membered heteroaryl, wherein each of the C₃-C₅ cycloalkyl, 3- to 8-membered heterocycloalkyl, C₆-C₁₀aryl or 5- to 10-membered heteroaryl is optionally further substituted with one or more of halogen, NH₂, CN, nitro, OH, C(O)OH, C₁-C₆alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₅ cycloalkyl, or 3- to 8-membered heterocycloalkyl, and R₂′, when not being H, is optionally substituted with one or more of halogen, N(Rₐ)₂, NHC(O)Rₐ, NHC(O)OR, OR_{b}, C₃-C₅ cycloalkyl, 3- to 8-membered heterocycloalkyl, C₆-C₁₀ aryl, or 5- to 10-membered heteroaryl, wherein each of the C₃-C₅ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆alkyl, C₁-C₆haloalkyl, C₁-C₆alkoxy, or C₁-C₆ haloalkoxy:
each R³ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3: and
n is 0, 1 or 2.

In some embodiments, LBM is

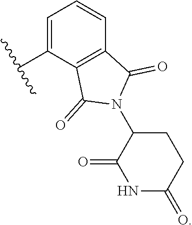 [SMILES: *c1cccc2c1C(=O)N(C1CCC(=O)NC1=O)C2=O]

In some embodiments, LBM is

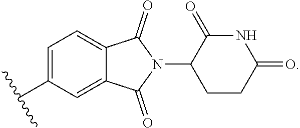 [SMILES: *c1ccc2c(c1)C(=O)N(C1CCC(=O)NC1=O)C2=O]

In some embodiments, LBM is

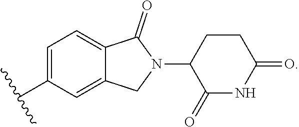 [SMILES: *c1ccc2c(c1)CN(C1CCC(=O)NC1=O)C2=O]

In some embodiments, LBM is

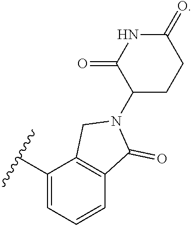 [SMILES: *c1cccc2c1CN(C1CCC(=O)NC1=O)C2=O]

In some embodiments, LBM is

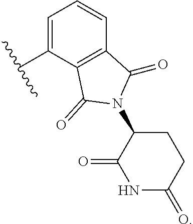 [SMILES: *c1cccc2c1C(=O)N([C@H]1CCC(=O)NC1=O)C2=O]

In some embodiments, LBM is

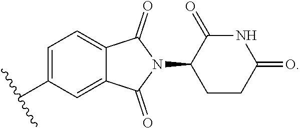 [SMILES: *c1ccc2c(c1)C(=O)N([C@@H]1CCC(=O)NC1=O)C2=O]

In some embodiments, LBM is

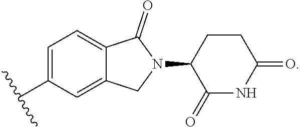 [SMILES: *c1ccc2c(c1)CN([C@H]1CCC(=O)NC1=O)C2=O]

In some embodiments, LBM is

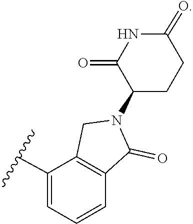 [SMILES: *c1cccc2c1CN([C@@H]1CCC(=O)NC1=O)C2=O]

In some embodiments, LBM is

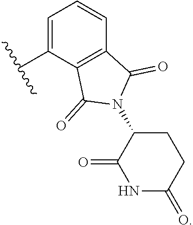 [SMILES: *c1cccc2c1C(=O)N([C@@H]1CCC(=O)NC1=O)C2=O]

In some embodiments, LBM is

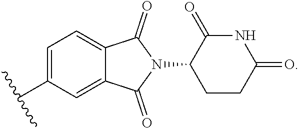 [SMILES: *c1ccc2c(c1)C(=O)N([C@H]1CCC(=O)NC1=O)C2=O]

In some embodiments, LBM is

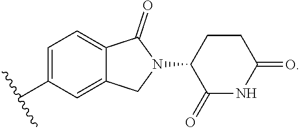 [SMILES: *c1ccc2c(c1)CN([C@@H]1CCC(=O)NC1=O)C2=O]

In some embodiments, LBM is

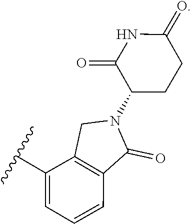 [SMILES: *c1cccc2c1CN([C@H]1CCC(=O)NC1=O)C2=O]

In some embodiments, LBM is

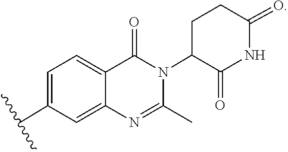 [SMILES: *c1ccc2c(=O)n(C3CCC(=O)NC3=O)c(C)nc2c1]

In some embodiments, LBM is

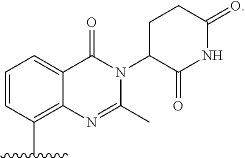 [SMILES: *c1cccc2c(=O)n(C3CCC(=O)NC3=O)c(C)nc12]

In some embodiments, LBM is

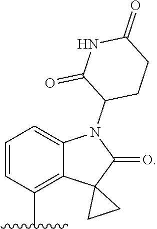 [SMILES: *c1cccc2c1C1(CC1)C(=O)N2C1CCC(=O)NC1=O]

In some embodiments, LBM is

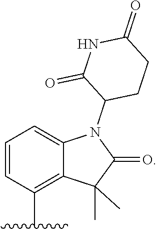 [SMILES: *c1cccc2c1C(C)(C)C(=O)N2C1CCC(=O)NC1=O]

selected from those in Table 1B below.

In certain embodiments, the present invention provides a compound of formula I, wherein LBM is an E3 ubiquitin ligase (cereblon) binding moiety thereby forming a compound of formula I-uu:

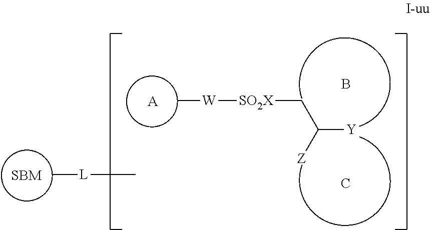

or a pharmaceutically acceptable salt thereof, wherein L and SBM are as defined above and described in embodiments herein, and wherein:
A represents a monocyclic or bicyclic aromatic ring which may be substituted;
B represents a six-membered unsaturated hydrocarbon ring or a six-membered unsaturated heterocycle containing one nitrogen atom as the heteroatom, each of which may be substituted;
C represents a five-membered heterocycle containing one or two nitrogen atoms which may be substituted;
W represents a single bond or a group represented by formula —CH═CH—;
X represents a group represented by formula —N(R¹)— or oxygen:
Y represents carbon or nitrogen:
Z represents a group represented by formula —N(R²)— or nitrogen: and
R¹ and R² may be the same or different from each other and each represent hydrogen or lower alkyl; as described and defined in U.S. Pat. No. 5,721,246, the entirety of each of which is herein incorporated by reference.

In some embodiments, LBM is a IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

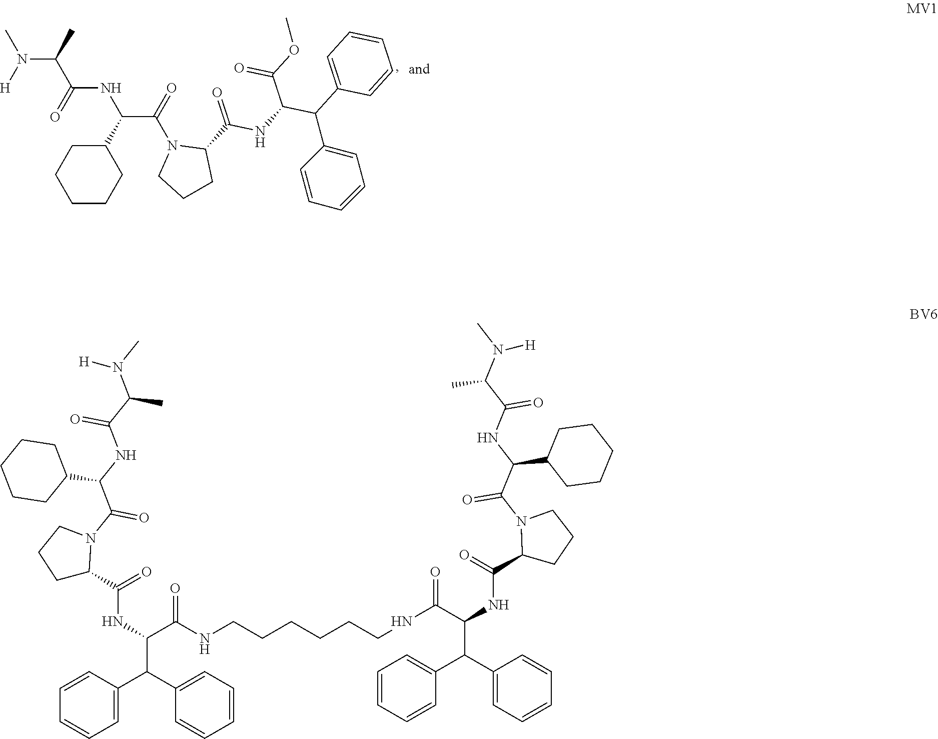 [SMILES: CN[C@@H](C)C(=O)N[C@H](C(=O)N1CCC[C@H]1C(=O)N[C@H](C(=O)OC)C(c1ccccc1)c1ccccc1)C1CCCCC1 ; CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

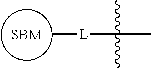

is attached to a modifiable carbon, oxygen, nitrogen or sulfur atom.

In certain embodiments, the present invention provides a compound of Formula I, wherein LBM is a MDM2 (i.e. human double minute 2 or HDM2) E3 ligase binding moiety thereby forming a compound of formula I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

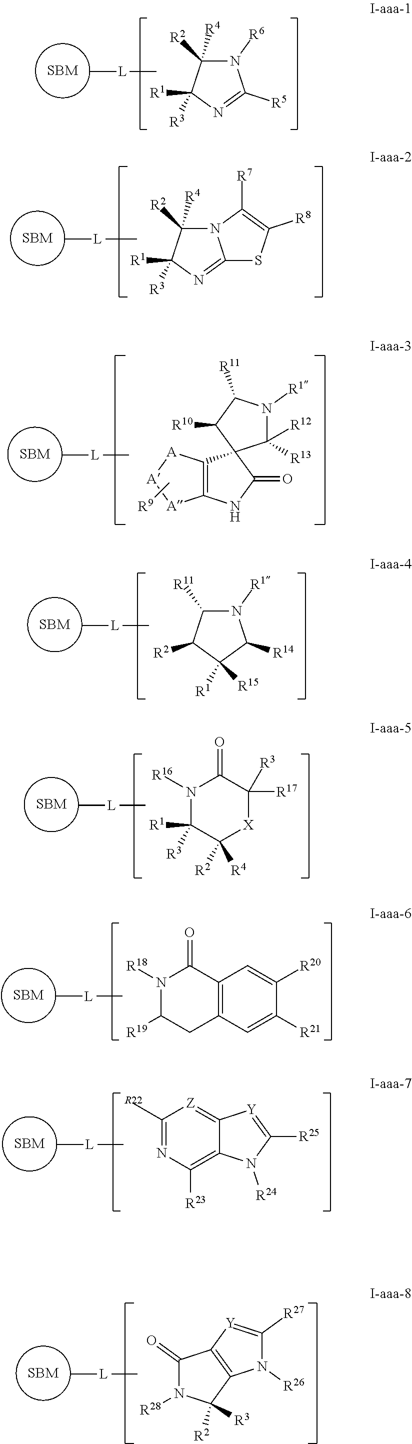

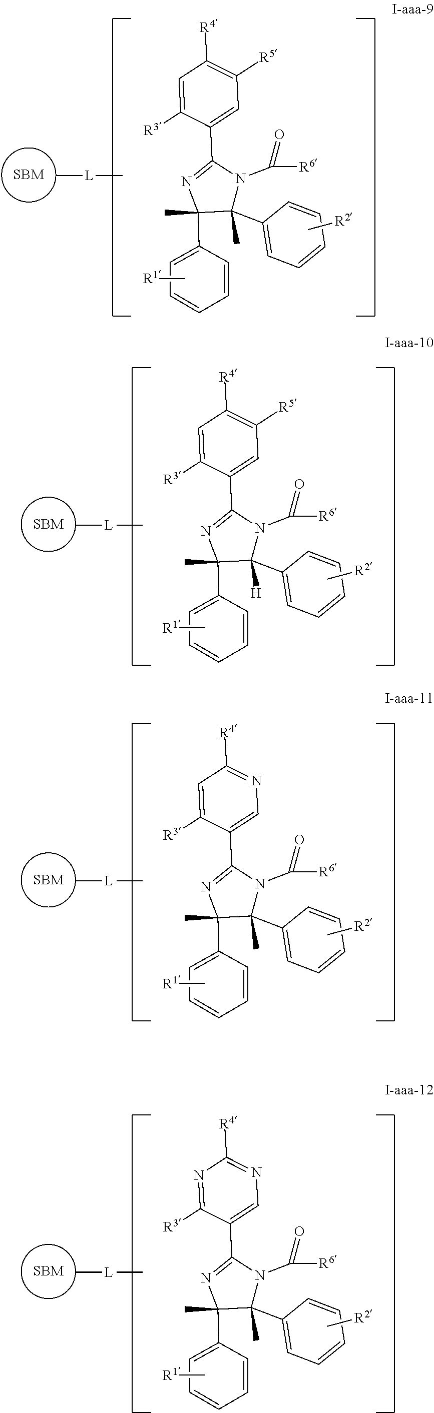

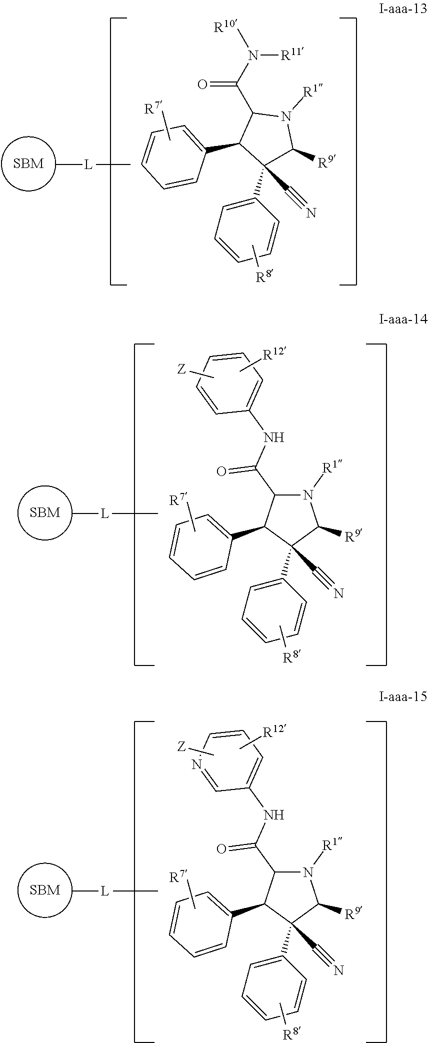

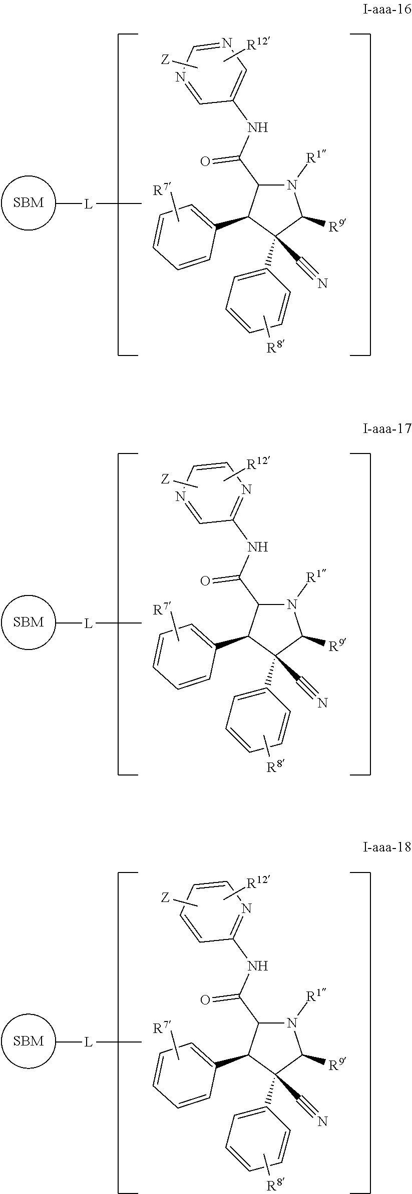

or a pharmaceutically acceptable salt thereof, wherein L and SBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen, and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic:
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R^{w} are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN:
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen. R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵ and R²⁷ are independently selected from hydrogen. R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C° CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C═CR, —NO₂, and —OR;
R^{9′} is R^{A};
Z¹ is selected from hydrogen, halogen, and —OR.
R^{10′} and R^{11′} are independently selected from hydrogen and R^{A}:
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of formula I-aaa-19, I-aaa-20, or I-aaa-21 respectively

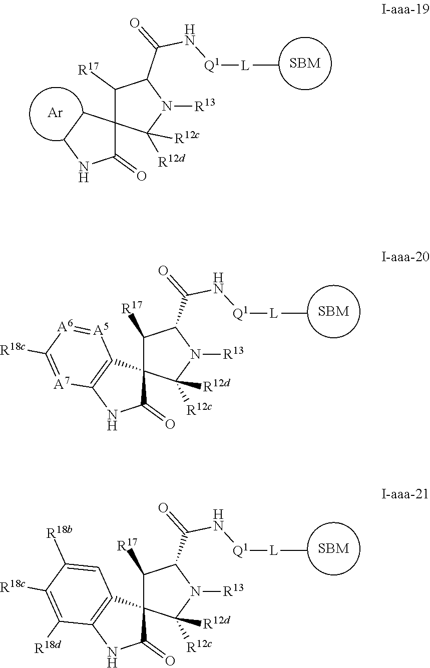

or a pharmaceutically acceptable salt thereof, wherein L and SBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R₁₂ and R₁₃ are each independently selected from hydrogen and R^{A}, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═;
A⁶ is selected from —C(R^{18b})═ and —N═;
A⁷ is selected from —C(R^{18d})═ and —N═:
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen, and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of Formula I, wherein LBM is an IAP E3 ubiquitin ligase binding moiety thereby forming a compound of formula I-bbb-1, I-bbb-2, I-bbb-3, or I-bbb-4 respectively:

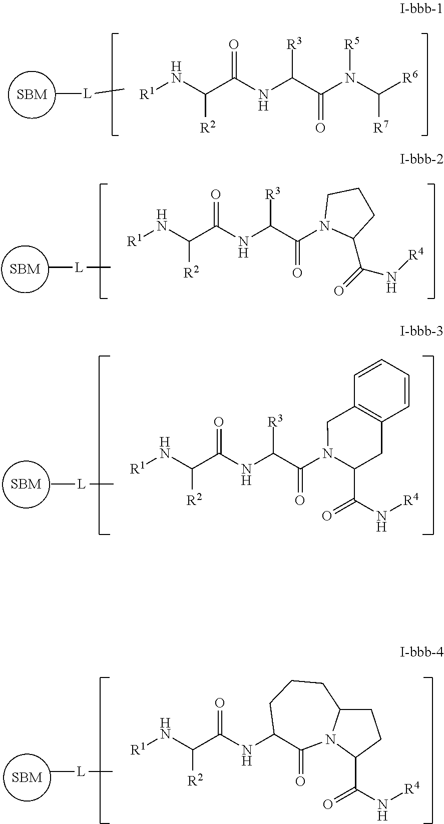

or a pharmaceutically acceptable salt thereof, wherein L and SBM are as defined above and described in embodiments herein, and wherein:
R¹ is selected from the group of H and alkyl;
R² is selected from the group of H and alkyl;
R³ is selected from the group of H, alkyl, cycloalkyl and heterocycloalkyl;
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above:
R⁵ and R⁶ are independently selected from the group of H, alkyl, cycloalkyl, heterocycloalkyl, aryl, heteroaryl or fused rings; and
R⁷ is selected from the group of cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above,
as defined and described in WO 2017/011590 and US 2017/0037004, the entirety of each of which is herein incorporated by reference.

In certain embodiments, the present invention provides a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1, I-ccc-2, or I-ccc-3:

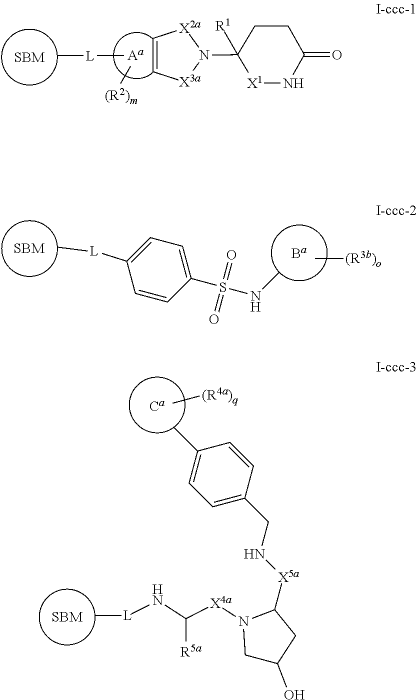

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂—, —C(O)—, —C(S)—,

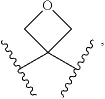 [SMILES: *C1(*)COC1]

CR₂CR₂, —N═CR—, or —CR═CR—;
each of X^{4a} and X^{5a} is independently a bivalent moiety selected from —CH₂—, —C(O)—, —C(S)—, or

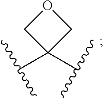 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3a}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic:
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1, wherein LBM is an E3 ubiquitin ligase (cereblon) binding moiety thereby forming a compound of formula I-ccc′-1 or I-ccc″-1:

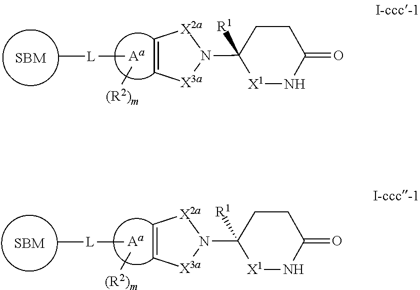

or a pharmaceutically acceptable salt thereof, wherein SBM, L, Ring A^{a}, X¹, X^{2a}, X^{3a}, R¹, R² and m are as described above.

In certain embodiments, the present invention provides a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1′, I-ccc-2′ or I-ccc-3′:

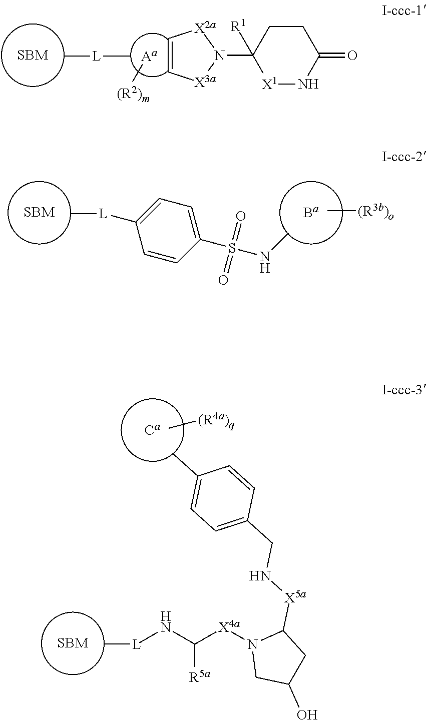

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂, —C(O)—, —C(S)—,

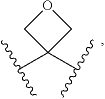 [SMILES: *C1(*)COC1]

-CR₂CR₂, —N═CR—, or —CR═CR—;
each of X^{4a} and X^{5a} is independently a bivalent moiety selected from —CH₂—, —C(O)—, —C(S)—, or

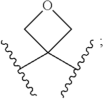 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic:
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; or:
   two R² groups of Ring A^{a} are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo: or a 5-10 membered heteroaryl having 1-4 heteroatoms independently selected from nitrogen, oxygen, or sulfur;
R^{5a} is hydrogen or C₁₋₆ aliphatic:
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5- to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1′, wherein LBM is an E3 ubiquitin ligase (cereblon) binding moiety thereby forming a compound of formula I-ccc′-1′ or I-ccc″-1′:

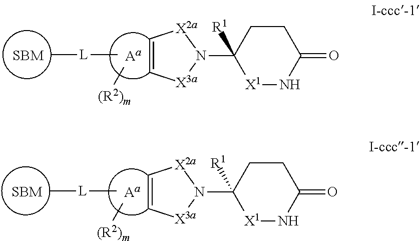

or a pharmaceutically acceptable salt thereof, wherein SBM, L, Ring A^{a}, X¹, X^{2a}, X^{3a}, R¹, R² and m are as described above.

In some embodiments, LBM is of formulae I-ccc-A, I-ccc-B, or I-ccc-C:

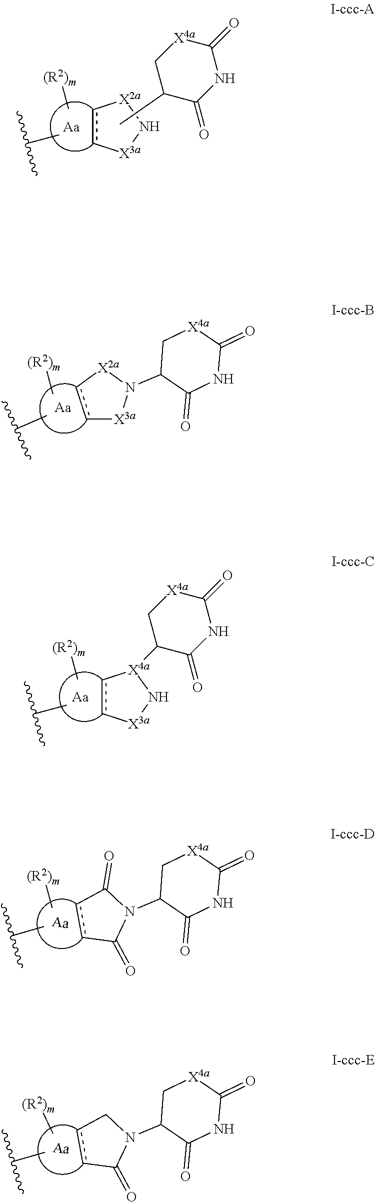

or a pharmaceutically acceptable salt thereof, wherein Ring A^{a}, X^{2a}, X^{3a}, R¹, R² and in are as defined above and described above; and:
X^{4a} is CH, CR, or N.

As defined above and described herein, each of X¹, X^{2a}, and X^{3a} is independently a bivalent moiety selected from a covalent bond, —CH₂—, —C(O)—, —C(S)—,

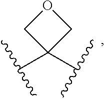 [SMILES: *C1(*)COC1]

—CR₂CR₂, —N═CR—, or —CR═CR—.

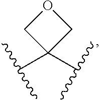 [SMILES: *C1(*)COC1]

In some embodiments, X¹ is a covalent bond, —CH₂—, —C(O)—, —C(S)—, CR₂CR₂, —N═CR—, or —CR—CR—.

In some embodiments, X¹ is selected from those depicted in Table 1B, below.

In some embodiments, X^{2a} is a covalent bond, —CH₂—, —C(O)—, —C(S)—, —CR₂CR₂, —N═CR—, or —CR═CR—. In some embodiments, X^{2a} is —C(O)—.

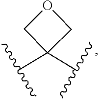 [SMILES: *C1(*)COC1]

In some embodiments, X^{2a} is selected from those depicted in Table 1B, below.

In some embodiments, X^{3a} is a covalent bond, —CH₂—, —C(O)—, —C(S)—, —CR₂CR₂, —N═CR—, or —CR═CR—. In some embodiments, X^{3a} is —C(O)—.

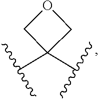 [SMILES: *C1(*)COC1]

In some embodiments, X²a and X^{3a} are —C(O)—. In some embodiments, X^{2a} is —C(O)—; and X³a is —CH₂—.

In some embodiments, X^{3a} is selected from those depicted in Table 1B, below.

As defined above and described herein. X^{4a} is CH, CR or N. In some embodiments, X^{4a} is CH. In some embodiments, X^{4a} is CR. In some embodiments, X^{4a} is N.

As defined above and described herein, each of X⁴ and X⁵ is independently a bivalent moiety selected from —CH₂—, —C(O)—, —C(S)—, or

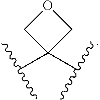 [SMILES: *C1(*)COC1]

In some embodiments, X^{4a} is —CH₂—, —C(O)—, —C(S)—, or

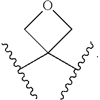 [SMILES: *C1(*)COC1]

In some embodiments, X^{4a} is selected from those depicted in Table 1B, below.

In some embodiments, X^{5a} is —CH₂—, —C(O)—, —C(S)—, or

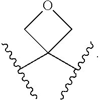 [SMILES: *C1(*)COC1]

In some embodiments, X^{5a} is selected from those depicted in Table 1B, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1B, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, two R² groups of Ring A^{a} are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R² groups of Ring A^{a} are taken together with their intervening atoms to form an optionally substituted ring selected from a 3-10 membered saturated or partially unsaturated carbocyclyl. In some embodiments, two R² groups of Ring A^{a} are taken together with their intervening atoms to form an optionally substituted ring selected from a 5-6 membered saturated or partially unsaturated carbocyclyl.

In some embodiments, two R² groups of Ring A^{a} are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R² groups of Ring A^{a} are taken together with their intervening atoms to form an optionally substituted benzo.

In some embodiments, two R² groups of Ring A^{a} are taken together with their intervening atoms to form an optionally substituted ring selected from a 5-10 membered heteroaryl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in Table 1B, below.

In some embodiments, R^{b} is hydrogen, —R⁶, halogen, —CN, —NO₂, —OR, —SR, —NR₂, —S(O)₂R, —S(O)₂NR—, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1B, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1B, below.

As defined above and described herein, R^{5a} is hydrogen or C₁₋₆ aliphatic.

In some embodiments, R^{w} is t-butyl.

In some embodiments, R^{5a} is selected from those depicted in Table 1B, below.

As defined above and described herein,

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur. In some embodiments, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is an optionally substituted 8-15 membered saturated or partially unsaturated tricyclic heterocyclylenyl having 1-5 heteroatoms independently selected from nitrogen, oxygen, and sulfur.

In some embodiments, R⁶ is a 12-membered saturated or partially unsaturated tricyclic heterocyclylenyl having 1-5 heteroatoms independently selected from nitrogen, oxygen, and sulfur. In some embodiments, R⁶ is a 12-membered saturated or partially unsaturated tricyclic heterocyclylenyl having 1-5 nitrogen heteroatoms. In some embodiments, R⁶ is a 12-membered saturated or partially unsaturated tricyclic heterocyclylenyl having 3 nitrogen heteroatoms.

In some embodiments, R⁶ is a 5,6,5-fused saturated or partially unsaturated tricyclic heterocyclylenyl having 1-5 heteroatoms independently selected from nitrogen, oxygen, and sulfur. In some embodiments, R⁶ is a 5,6,5-fused membered saturated or partially unsaturated tricyclic heterocyclylenyl having 1-5 nitrogen heteroatoms. In some embodiments, R⁶ is a 5,6,5-fused membered saturated or partially unsaturated tricyclic heterocyclylenyl having 3 nitrogen heteroatoms.

In some embodiments, R⁶ is a 5,5,6-fused saturated or partially unsaturated tricyclic heterocyclylenyl having 1-5 heteroatoms independently selected from nitrogen, oxygen, and sulfur. In some embodiments, R⁶ is a 5,5,6-fused membered saturated or partially unsaturated tricyclic heterocyclylenyl having 1-5 nitrogen heteroatoms. In some embodiments, R⁶ is a 5,5,6-fused membered saturated or partially unsaturated tricyclic heterocyclylenyl having 3 nitrogen heteroatoms.

In some embodiments, R⁶ is a 13-membered saturated or partially unsaturated tricyclic heterocyclylenyl having 1-5 heteroatoms independently selected from nitrogen, oxygen, and sulfur. In some embodiments, R⁶ is a 13-membered saturated or partially unsaturated tricyclic heterocyclylenyl having 1-5 nitrogen heteroatoms. In some embodiments, R⁶ is a 13-membered saturated or partially unsaturated tricyclic heterocyclylenyl having 3 nitrogen heteroatoms.

In some embodiments, R⁶ is a 5,6,6-fused saturated or partially unsaturated tricyclic heterocyclylenyl having 1-5 heteroatoms independently selected from nitrogen, oxygen, and sulfur. In some embodiments, R⁶ is a 5,6,6-fused membered saturated or partially unsaturated tricyclic heterocyclylenyl having 1-5 nitrogen heteroatoms. In some embodiments, R⁶ is a 5,6,6-fused membered saturated or partially unsaturated tricyclic heterocyclylenyl having 3 nitrogen heteroatoms.

In some embodiments, R⁶ is a 6,5,6-fused saturated or partially unsaturated tricyclic heterocyclylenyl having 1-5 heteroatoms independently selected from nitrogen, oxygen, and sulfur. In some embodiments, R⁶ is a 6,5,6-fused membered saturated or partially unsaturated tricyclic heterocyclylenyl having 1-5 nitrogen heteroatoms. In some embodiments, R⁶ is a 6,5,6-fused membered saturated or partially unsaturated tricyclic heterocyclylenyl having 3 nitrogen heteroatoms.

In some embodiments, R⁶ is a 14-membered saturated or partially unsaturated tricyclic heterocyclylenyl having 1-5 heteroatoms independently selected from nitrogen, oxygen, and sulfur.

In some embodiments, R⁶ is or an 10-15 membered tricyclic heteroarylenyl having 1-5 heteroatoms independently selected from nitrogen, oxygen, and sulfur.

In some embodiments, R⁶ is a 12-membered saturated or partially unsaturated tricyclic heteroarylenyl having 1-5 heteroatoms independently selected from nitrogen, oxygen, and sulfur. In some embodiments, R⁶ is a 12-membered saturated or partially unsaturated tricyclic heteroarylenyl having 1-5 nitrogen heteroatoms. In some embodiments, R⁶ is a 12-membered saturated or partially unsaturated tricyclic heteroarylenyl having 3 nitrogen heteroatoms.

In some embodiments, R⁶ is a 5,6,5-fused saturated or partially unsaturated tricyclic heteroarylenyl having 1-5 heteroatoms independently selected from nitrogen, oxygen, and sulfur. In some embodiments, R⁶ is a 5,6,5-fused membered saturated or partially unsaturated tricyclic heteroarylenyl having 1-5 nitrogen heteroatoms. In some embodiments, R⁶ is a 5,6,5-fused membered saturated or partially unsaturated tricyclic heteroarylenyl having 3 nitrogen heteroatoms.

In some embodiments, R⁶ is a 5,5,6-fused saturated or partially unsaturated tricyclic heteroarylenyl having 1-5 heteroatoms independently selected from nitrogen, oxygen, and sulfur. In some embodiments, R⁶ is a 5,5,6-fused membered saturated or partially unsaturated tricyclic heteroarylenyl having 1-5 nitrogen heteroatoms. In some embodiments, R⁶ is a 5,5,6-fused membered saturated or partially unsaturated tricyclic heteroarylenyl having 3 nitrogen heteroatoms.

In some embodiments, R⁶ is a 13-membered saturated or partially unsaturated tricyclic heteroarylenyl having 1-5 heteroatoms independently selected from nitrogen, oxygen, and sulfur. In some embodiments, R⁶ is a 13-membered saturated or partially unsaturated tricyclic heteroarylenyl having 1-5 nitrogen heteroatoms. In some embodiments, R⁶ is a 13-membered saturated or partially unsaturated tricyclic heteroarylenyl having 3 nitrogen heteroatoms.

In some embodiments, R⁶ is a 5,6,6-fused saturated or partially unsaturated tricyclic heteroarylenyl having 1-5 heteroatoms independently selected from nitrogen, oxygen, and sulfur. In some embodiments, R⁶ is a 5,6,6-fused membered saturated or partially unsaturated tricyclic heteroarylenyl having 1-5 nitrogen heteroatoms. In some embodiments, R⁶ is a 5,6,6-fused membered saturated or partially unsaturated tricyclic heteroarylenyl having 3 nitrogen heteroatoms.

In some embodiments, R⁶ is a 6,5,6-fused saturated or partially unsaturated tricyclic heteroarylenyl having 1-5 heteroatoms independently selected from nitrogen, oxygen, and sulfur. In some embodiments, R⁶ is a 6,5,6-fused membered saturated or partially unsaturated tricyclic heteroarylenyl having 1-5 nitrogen heteroatoms. In some embodiments, R⁶ is a 6,5,6-fused membered saturated or partially unsaturated tricyclic heteroarylenyl having 3 nitrogen heteroatoms.

In some embodiments, R⁶ is selected from those depicted in Table 1B, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen or sulfur. In some embodiments Ring A^{a} is a fused 5-membered heteroaryl with 1-3 heteroatoms independently selected from nitrogen, oxygen or sulfur.

In some embodiments, Ring A^{a} is a fused phenyl.

In some embodiments, Ring A^{a} is selected from those depicted in Table 1B, below.

As defined above and described herein, Ring Ba is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, or sulfur.

In some embodiments, Ring B^{a} is

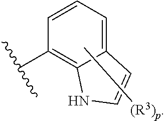

In some embodiments, Ring B^{a} is selected from those depicted in Table 1B, below.

As defined above and described herein, Ring C^{a} is selected from 6-membered aryl containing 0-2 nitrogen atoms or a 5-membered heteroaryl with 1-3 heteroatoms independently selected from nitrogen, oxygen or sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen or sulfur.

In some embodiments, Ring C^{a} is

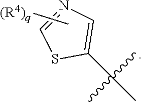

In some embodiments, Ring C^{a} is selected from those depicted in Table 1B, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1B, below.

In some embodiments, o is selected from those depicted in Table 1B, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1B, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1B, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1B, below.

In certain embodiments, the present invention provides a compound of formula I, wherein LBM is a VHL E3 ubiquitin ligase binding moiety, thereby forming a compound of formula I-ddd:

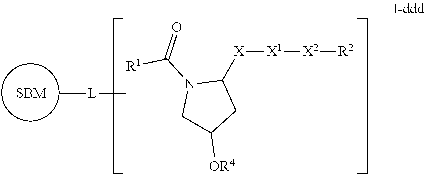

or a pharmaceutically acceptable salt thereof, wherein L and SBM is as defined above and described in embodiments herein, and wherein:
X is —C(O)—, —C(O)NR—, —SO₂—, —SO₂NR—, or an optionally substituted 5-membered heterocyclic ring:
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen, halogen, —CN, or

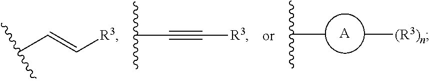

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or two R³ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

In certain embodiments, the present invention provides a compound of formula I, wherein LBM is an IAP binding moiety thereby forming a compound of formula I-fff:

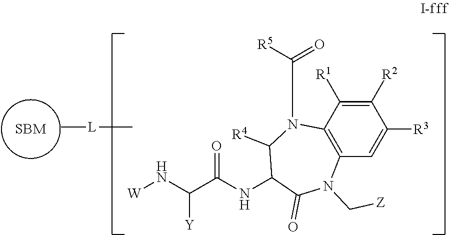

or a pharmaceutically acceptable salt thereof, wherein L and SBM are as defined above and described in embodiments herein, and wherein:
W is selected from H and lower alkyl that optionally may be substituted with 1-3 deuterium atoms;
Y is lower alkyl that optionally may be substituted with OR⁶;
R¹, R² and R³ are the same or different and each is independently selected from H and cyano;
R⁴ is lower alkyl;
R⁵ is selected from the group a) lower alkyl that optionally may be substituted with SO₂R⁶ and OR⁶, b) heterocyclyl, and c) aryl that optionally may be substituted with C(O)R⁷, halo and cyano;
Z is selected from the group a) aryl that optionally may be substituted with lower alkyl, OR⁶, halogen and aryl that optionally may be substituted with halogen, b) heteroaryl that optionally may be substituted with lower alkyl, cycloalkyl, OR, halogen, oxo and aryl that optionally may substituted with cyano, and c) aryl fused with heterocyclyl, wherein the aryl optionally may be substituted with OR⁶ and halogen, and the heterocyclyl optionally may be substituted with oxo, and d) heterocyclyl;
R⁶ is selected from H and lower alkyl that optionally may be substituted with halogen and deuterium; and
R⁷ is lower alkyl,
as described and defined in WO 2014/044622, US 2015/0225449, WO 2015/071393, and US 2016/0272596, the entirety of each of which is herein incorporated by reference.

In certain embodiments, the present invention provides a compound of formula I, wherein LBM is a MDM2 binding moiety thereby forming a compound of formula I-ggg:

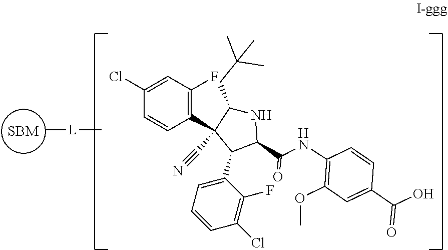

or a pharmaceutically acceptable salt thereof, wherein L and SBM are as defined above and described in embodiments herein, as described and defined in Hines, J. et al., Cancer Res. (DOI: 10.1158/0008-5472.CAN-18-2918), the entirety of each of which is herein incorporated by reference.

In certain embodiments, the present invention provides a compound of formula I, wherein LBM is a DCAF16 binding moiety thereby forming a compound of formula I-hhh:

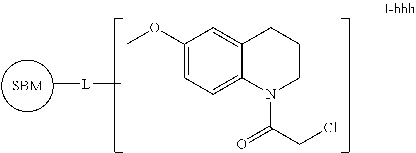

or a pharmaceutically acceptable salt thereof, wherein L and SBM are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides a compound of formula I, wherein LBM is a RNF114 binding moiety thereby forming a compound of formula I-iii:

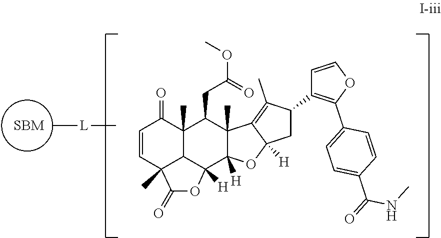

or a pharmaceutically acceptable salt thereof, wherein L and SBM are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides a compound of formula I, wherein LBM is a RNF4 binding moiety thereby forming a compound of formula I-jjj:

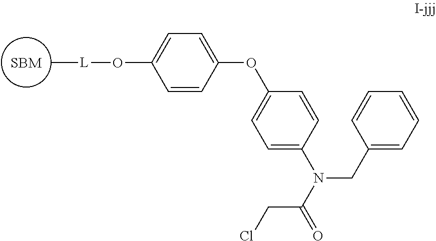

or a pharmaceutically acceptable salt thereof, wherein L and SBM are as defined above and described in embodiments herein, as described and defined in Ward, C. C., et al., bioRxiv (doi: https://doi.org.10.1101/439125), the entirety of each of which is herein incorporated by reference.

In some embodiments, LBM is

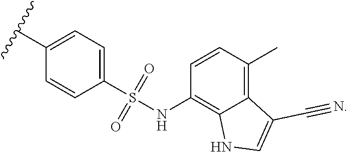 [SMILES: *c1ccc(S(=O)(=O)Nc2ccc(C)c3c(C#N)c[nH]c23)cc1]

In some embodiments, LBM is

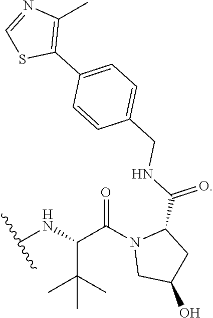 [SMILES: *N[C@H](C(=O)N1C[C@H](O)C[C@H]1C(=O)NCc1ccc(-c2scnc2C)cc1)C(C)(C)C]

In some embodiments, LBM is

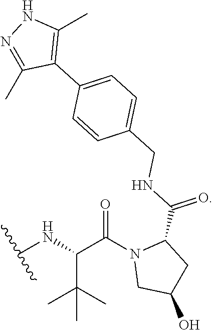 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

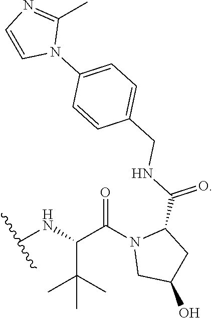 [SMILES: *N[C@H](C(=O)N1C[C@H](O)C[C@H]1C(=O)NCc1ccc(-n2ccnc2C)cc1)C(C)(C)C]

In some embodiments, LBM is

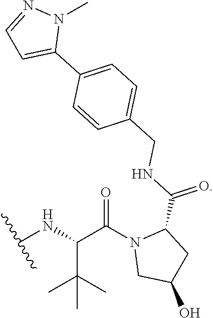 [SMILES: *N[C@H](C(=O)N1C[C@H](O)C[C@H]1C(=O)NCc1ccc(-c2ccnn2C)cc1)C(C)(C)C]

In some embodiments, LBM is

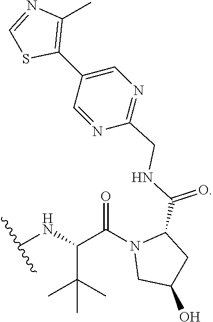 [SMILES: *N[C@H](C(=O)N1C[C@H](O)C[C@H]1C(=O)NCc1ncc(-c2scnc2C)cn1)C(C)(C)C]

In some embodiments, LBM is

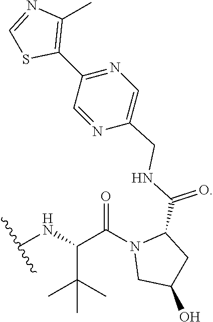 [SMILES: *N[C@H](C(=O)N1C[C@H](O)C[C@H]1C(=O)NCc1cnc(-c2scnc2C)cn1)C(C)(C)C]

In some embodiments, LBM is

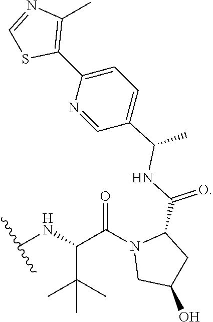 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

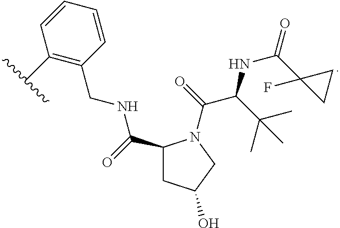 [SMILES: *c1ccccc1CNC(=O)[C@@H]1C[C@@H](O)CN1C(=O)[C@@H](NC(=O)C1(F)CC1)C(C)(C)C]

In some embodiments, LBM is

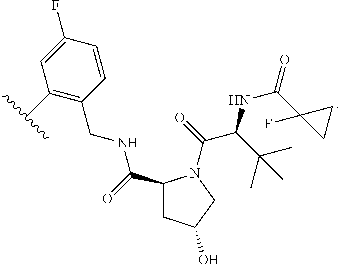 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

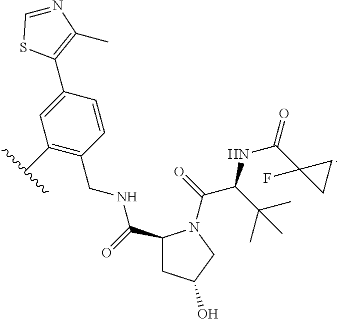 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

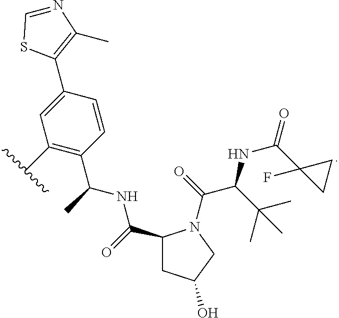 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C]

In some embodiments, LBM is

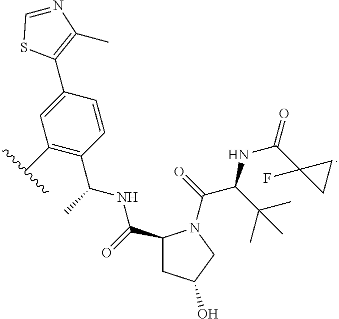 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

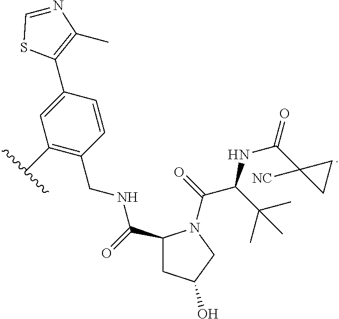 [SMILES: *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, LBM is

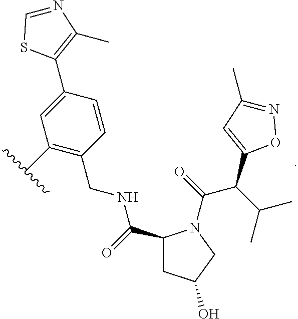 [SMILES: *c1cc(-c2scnc2C)ccc1CNC(=O)[C@@H]1C[C@@H](O)CN1C(=O)[C@@H](c1cc(C)no1)C(C)C]

In some embodiments, LBM is

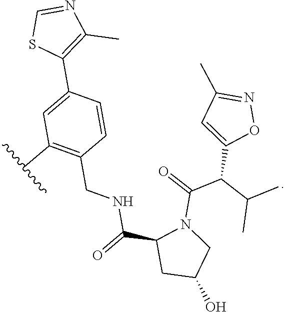 [SMILES: *c1cc(-c2scnc2C)ccc1CNC(=O)[C@@H]1C[C@@H](O)CN1C(=O)[C@H](c1cc(C)no1)C(C)C]

In some embodiments, LBM is

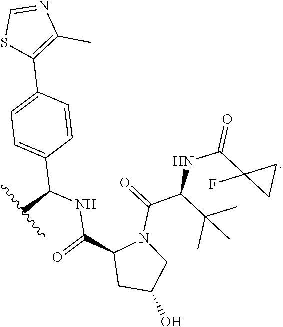 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

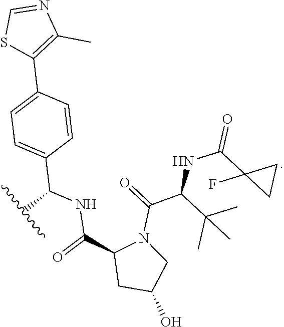 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

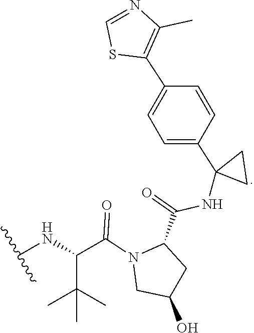 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

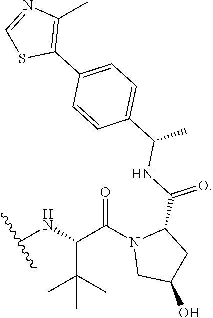 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

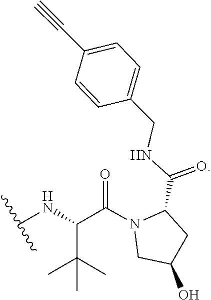 [SMILES: *N[C@H](C(=O)N1C[C@H](O)C[C@H]1C(=O)NCc1ccc(C#C)cc1)C(C)(C)C]

In some embodiments, LBM is

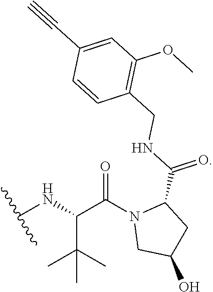 [SMILES: *N[C@H](C(=O)N1C[C@H](O)C[C@H]1C(=O)NCc1ccc(C#C)cc1OC)C(C)(C)C]

In some embodiments, LBM is

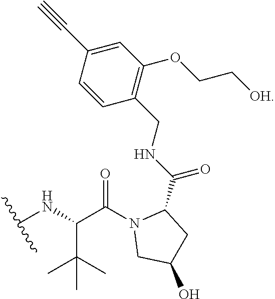 [SMILES: *N[C@H](C(=O)N1C[C@H](O)C[C@H]1C(=O)NCc1ccc(C#C)cc1OCCO)C(C)(C)C]

In some embodiments, LBM is

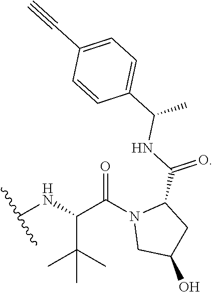 [SMILES: *N[C@H](C(=O)N1C[C@H](O)C[C@H]1C(=O)N[C@@H](C)c1ccc(C#C)cc1)C(C)(C)C]

In some embodiments, LBM is

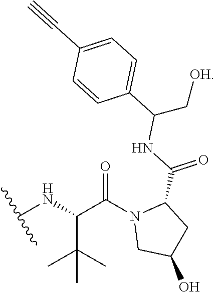 [SMILES: *N[C@H](C(=O)N1C[C@H](O)C[C@H]1C(=O)NC(CO)c1ccc(C#C)cc1)C(C)(C)C]

In some embodiments, LBM is

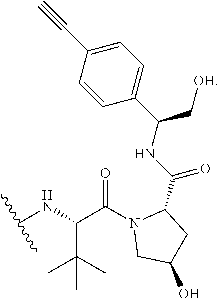 [SMILES: *N[C@H](C(=O)N1C[C@H](O)C[C@H]1C(=O)N[C@H](CO)c1ccc(C#C)cc1)C(C)(C)C]

In some embodiments, LBM is

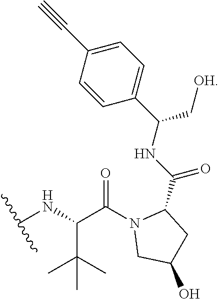 [SMILES: *N[C@H](C(=O)N1C[C@H](O)C[C@H]1C(=O)N[C@@H](CO)c1ccc(C#C)cc1)C(C)(C)C]

In some embodiments, LBM is

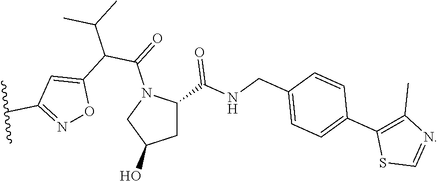 [SMILES: *c1cc(C(C(=O)N2C[C@H](O)C[C@H]2C(=O)NCc2ccc(-c3scnc3C)cc2)C(C)C)on1]

In some embodiments, LBM is

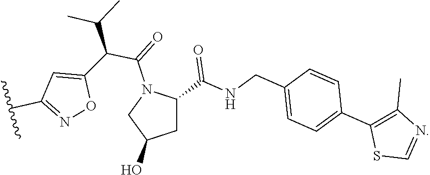 [SMILES: *c1cc([C@H](C(=O)N2C[C@H](O)C[C@H]2C(=O)NCc2ccc(-c3scnc3C)cc2)C(C)C)on1]

In some embodiments, LBM is

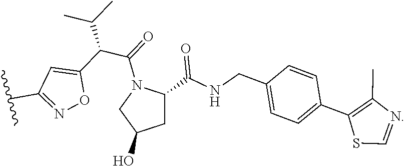 [SMILES: *c1cc([C@@H](C(=O)N2C[C@H](O)C[C@H]2C(=O)NCc2ccc(-c3scnc3C)cc2)C(C)C)on1]

In some embodiments, LBM is

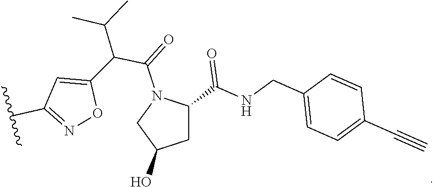 [SMILES: *c1cc(C(C(=O)N2C[C@H](O)C[C@H]2C(=O)NCc2ccc(C#C)cc2)C(C)C)on1]

In some embodiments, LBM is

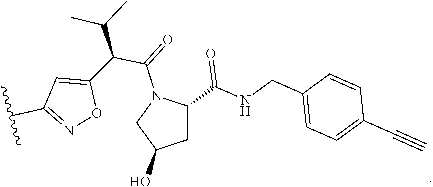 [SMILES: *c1cc([C@H](C(=O)N2C[C@H](O)C[C@H]2C(=O)NCc2ccc(C#C)cc2)C(C)C)on1]

In some embodiments, LBM is

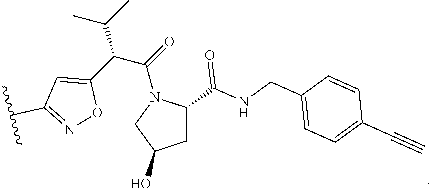 [SMILES: *c1cc([C@@H](C(=O)N2C[C@H](O)C[C@H]2C(=O)NCc2ccc(C#C)cc2)C(C)C)on1]

In some embodiments, LBM is

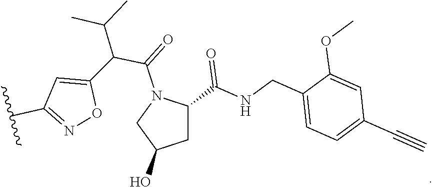 [SMILES: *c1cc(C(C(=O)N2C[C@H](O)C[C@H]2C(=O)NCc2ccc(C#C)cc2OC)C(C)C)on1]

In some embodiments, LBM is

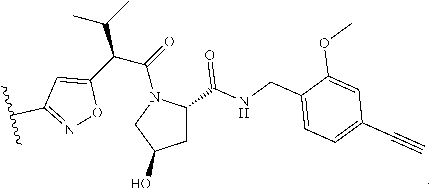 [SMILES: *c1cc([C@H](C(=O)N2C[C@H](O)C[C@H]2C(=O)NCc2ccc(C#C)cc2OC)C(C)C)on1]

In some embodiments, LBM is

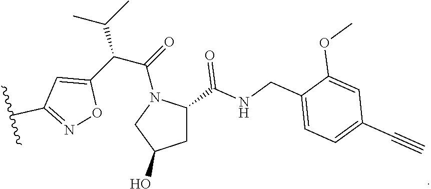 [SMILES: *c1cc([C@@H](C(=O)N2C[C@H](O)C[C@H]2C(=O)NCc2ccc(C#C)cc2OC)C(C)C)on1]

In some embodiments, LBM is

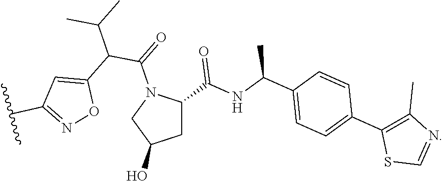 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

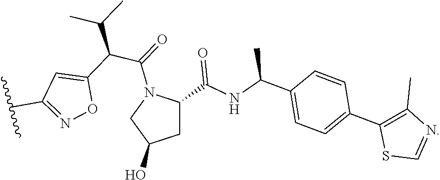 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

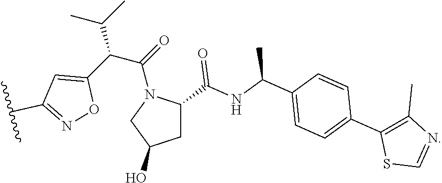 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

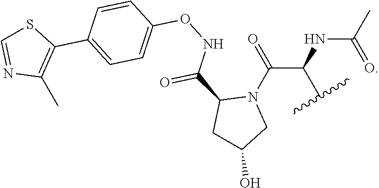 [SMILES: *[C@H](NC(C)=O)C(=O)N1C[C@H](O)C[C@H]1C(=O)NOc1ccc(-c2scnc2C)cc1]

In some embodiments, LBM is

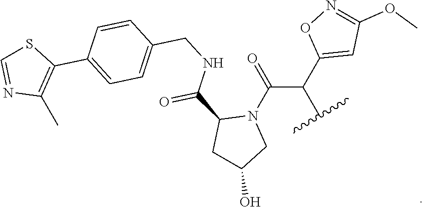 [SMILES: *C(C(=O)N1C[C@H](O)C[C@H]1C(=O)NCc1ccc(-c2scnc2C)cc1)c1cc(OC)no1]

In some embodiments, LBM is

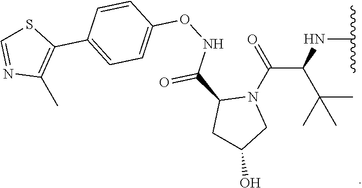 [SMILES: *N[C@H](C(=O)N1C[C@H](O)C[C@H]1C(=O)NOc1ccc(-c2scnc2C)cc1)C(C)(C)C]

In some embodiments, LBM is

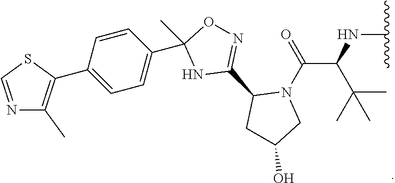 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

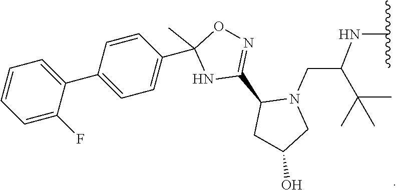 [SMILES: *NC(CN1C[C@H](O)C[C@H]1C1=NOC(C)(c2ccc(-c3ccccc3F)cc2)N1)C(C)(C)C]

In some embodiments, LBM is

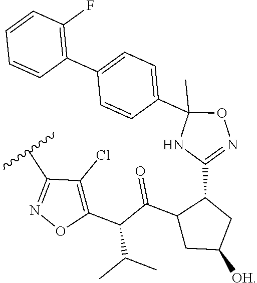 [SMILES: *c1noc([C@H](C(=O)C2C[C@H](O)C[C@H]2C2=NOC(C)(c3ccc(-c4ccccc4F)cc3)N2)C(C)C)c1Cl]

In some embodiments, LBM is

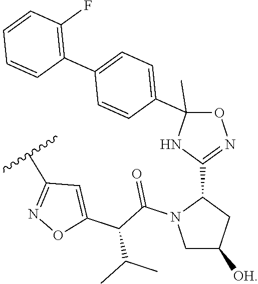 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

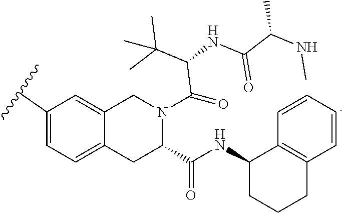 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

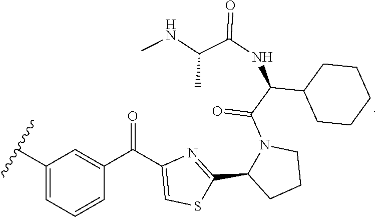 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

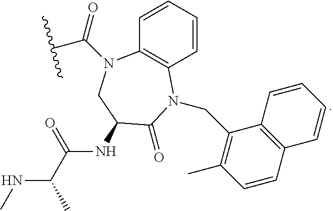 [SMILES: *C(=O)N1C[C@H](NC(=O)[C@H](C)NC)C(=O)N(Cc2c(C)ccc3ccccc23)c2ccccc21]

In some embodiments, LBM is

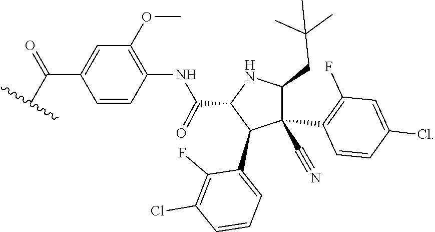 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

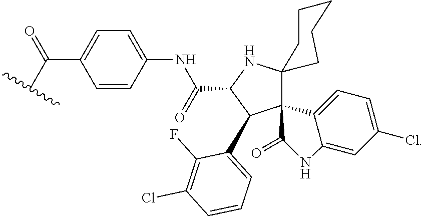 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

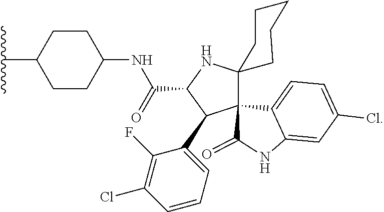 [SMILES: *C1CCC(NC(=O)[C@@H]2NC3(CCCCC3)[C@@]3(C(=O)Nc4cc(Cl)ccc43)[C@H]2c2cccc(Cl)c2F)CC1]

In some embodiments, LBM is

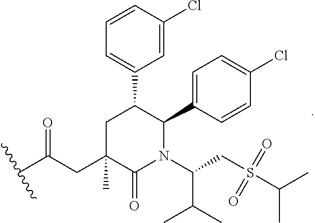 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

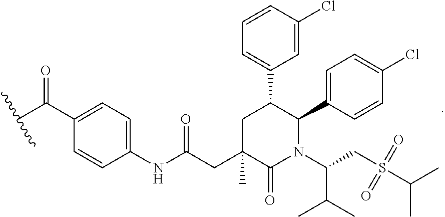 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

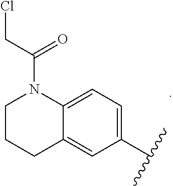 [SMILES: *c1ccc2c(c1)CCCN2C(=O)CCl]

In some embodiments, LBM is

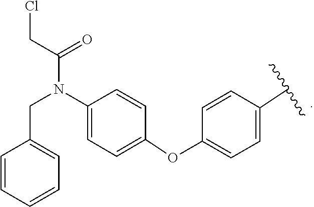 [SMILES: *c1ccc(Oc2ccc(N(Cc3ccccc3)C(=O)CCl)cc2)cc1]

In some embodiments, LBM is

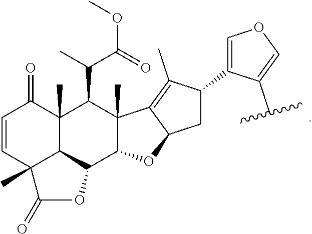 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

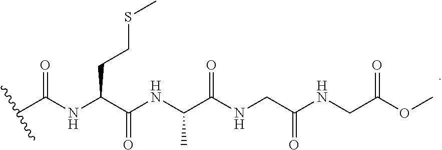 [SMILES: *C(=O)N[C@@H](CCSC)C(=O)N[C@@H](C)C(=O)NCC(=O)NCC(=O)OC]

In some embodiments, LBM is

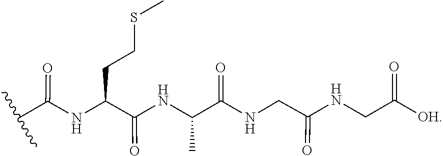 [SMILES: *C(=O)N[C@@H](CCSC)C(=O)N[C@@H](C)C(=O)NCC(=O)NCC(=O)O]

In some embodiments, LBM is

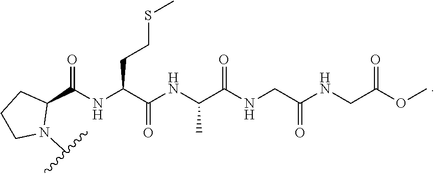 [SMILES: *N1CCC[C@H]1C(=O)N[C@@H](CCSC)C(=O)N[C@@H](C)C(=O)NCC(=O)NCC(=O)OC]

In some embodiments, LBM is

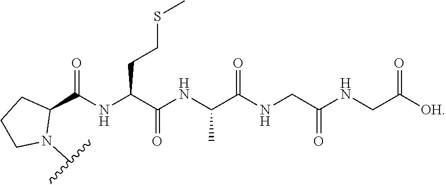 [SMILES: *N1CCC[C@H]1C(=O)N[C@@H](CCSC)C(=O)N[C@@H](C)C(=O)NCC(=O)NCC(=O)O]

In some embodiments, LBM is

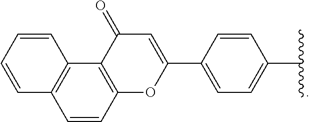 [SMILES: *c1ccc(-c2cc(=O)c3c(ccc4ccccc43)o2)cc1]

In some embodiments, LBM is

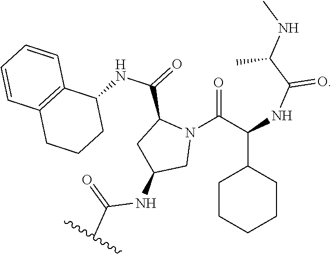 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

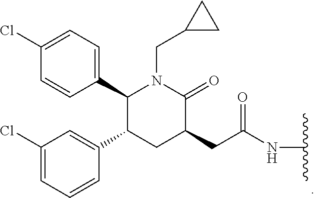 [SMILES: *NC(=O)C[C@H]1C[C@H](c2cccc(Cl)c2)[C@@H](c2ccc(Cl)cc2)N(CC2CC2)C1=O]

In some embodiments, LBM is

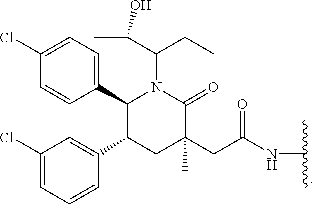 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

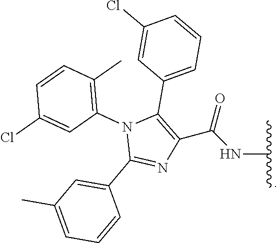 [SMILES: *NC(=O)c1nc(-c2cccc(C)c2)n(-c2cc(Cl)ccc2C)c1-c1cccc(Cl)c1]

In some embodiments, LBM is

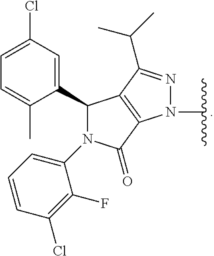 [SMILES: *n1nc(C(C)C)c2c1C(=O)N(c1cccc(Cl)c1F)[C@H]2c1cc(Cl)ccc1C]

In some embodiments, LBM is

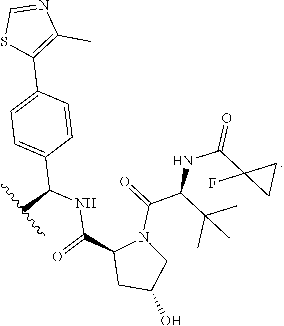 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In certain embodiments, the present invention provides a compound of formula I, wherein LBM is a CRBN E3 ubiquitin ligase binding moiety thereby forming a compound of formula I-qqq:

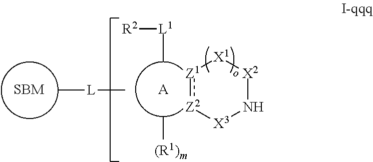

or a pharmaceutically acceptable salt thereof, wherein L and SBM are as defined above and described in embodiments herein, wherein:
each X¹ is independently —CH₂—, —O—, —NR—, —CF₂—,

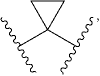 [SMILES: *C1(*)CC1]

—C(O)—, —C(S)—, or

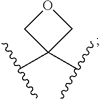 [SMILES: *C1(*)COC1]

X² and X³ are independently —CH₂—, —C(O)—, —C(S)—, or

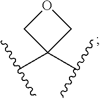 [SMILES: *C1(*)COC1]

Z¹ and Z² are independently a carbon atom or a nitrogen atom;
   Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₃—:
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

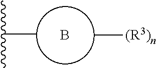

or hydrogen;
Ring B is phenyl, a 4-10 membered saturated or partially unsaturated mono- or bicyclic carbocyclic or heterocylic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
o is 0, 1, or 2.

As defined above and described herein each X¹ is independently a covalent bond, —CH₂—, —O—, —NR—, —CF₂—,

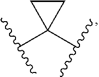 [SMILES: *C1(*)CC1]

### —C(O)—, —C(S)—, or

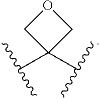 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

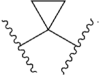 [SMILES: *C1(*)CC1]

In some embodiments, X¹ is —C(O)—. In some embodiments, X¹ is —C(S)—. In some embodiments X¹ is

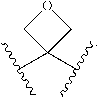 [SMILES: *C1(*)COC1]

In certain embodiments, X¹ is selected from those shown in the compounds of Table 1B.

As defined above and described herein, X² and X³ are independently —CH₂—, —C(O)—, —C(S)—, or

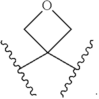 [SMILES: *C1(*)COC1]

In some embodiments, X² and X³ are independently —CH₂—. In some embodiments, X² and X³ are independently —C(O)—. In some embodiments, X² and X are independently —C(S)—. In some embodiments, X² and X³ are independently

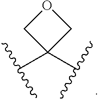 [SMILES: *C1(*)COC1]

In certain embodiments, X² and X³ are independently selected from those shown in the compounds of Table 1B.

As defined above and described herein. X⁴ is a covalent bond, —CH₂—, —CR₂—, —O—, —NR—, —CF₂—,

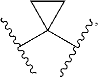 [SMILES: *C1(*)CC1]

### —C(O)—, —C(S)—, or

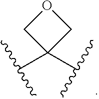 [SMILES: *C1(*)COC1]

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1B.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In certain embodiments, Ring A is selected from those shown in the compounds of Table 1B.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1B.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In certain embodiments, each R¹ is independently selected from those shown in the compounds of Table 1B.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

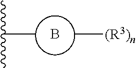

or hydrogen.

In some embodiment R² is

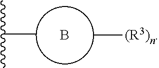

In some embodiments, R² is hydrogen.

In certain embodiments, R² is selected from those shown in the compounds of Table 1B.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table 1B.

As defined above and described herein, each R³ is independently selected from hydrogen, deuterium. R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1B.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1B.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1B.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1B.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1B.

As defined above and described herein, o is 0, 1, or 2.

In some embodiments, n is 0. In some embodiments, n is 1. In some embodiments, m is 2.

In certain embodiments, o is selected from those shown in the compounds of Table 1B.

In some embodiments, the present invention provides a compound of formula I-qqq-A:

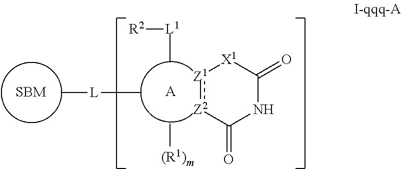

or a pharmaceutically acceptable salt thereof, wherein each of SBM, L, L¹, R¹, R², m, Z¹, Z², and X¹ is as defined above and described in embodiments herein, both singly and in combination.

In some embodiments, the present invention provides a compound of formula I-qqq-B:

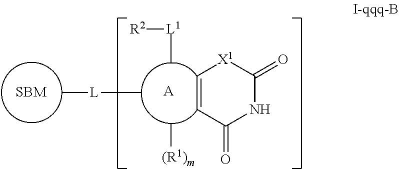

or a pharmaceutically acceptable salt thereof, wherein each of SBM, L, L¹, R¹, R², m, and X¹ is as defined above and described in embodiments herein, both singly and in combination.

In some embodiments, the present invention provides a compound of formula I-qqq, wherein Ring A is benzo, o is 1, X¹ is —CH₂—, X² and X³ are —C(O)—, and Z¹ and Z² are carbon atoms as shown, to provide a compound of formula I-qqq-1:

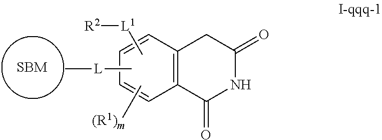

or a pharmaceutically acceptable salt thereof, wherein each of SBM, L, L¹, R¹, R², and m is as defined above and described in embodiments herein, both singly and in combination.

In some embodiments, the present invention provides a compound of formula I-qqq, wherein Ring A is benzo, o is 1, X¹, X² and X³ are —C(O)—, and Z¹ and Z² are carbon atoms as shown, to provide a compound of formula I-qqq-12:

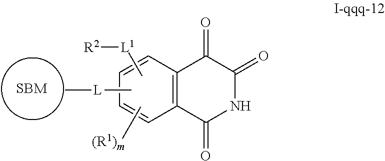

or a pharmaceutically acceptable salt thereof, wherein each of SBM, L, L¹, R¹, R², and m is as defined above and described in embodiments herein, both singly and in combination.

In some embodiments, LBM is

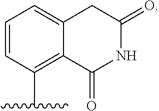 [SMILES: *c1cccc2c1C(=O)NC(=O)C2]

In some embodiments, LBM is

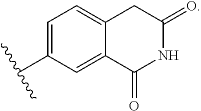 [SMILES: *c1ccc2c(c1)C(=O)NC(=O)C2]

In some embodiments, LBM is

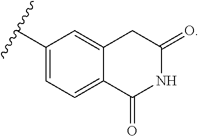 [SMILES: *c1ccc2c(c1)CC(=O)NC2=O]

In some embodiments, LBM is

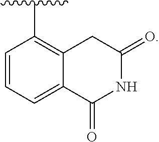 [SMILES: *c1cccc2c1CC(=O)NC2=O]

In some embodiments, LBM is

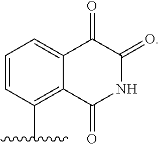 [SMILES: *c1cccc2c1C(=O)NC(=O)C2=O]

In some embodiments, LBM is

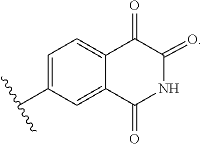 [SMILES: *c1ccc2c(c1)C(=O)NC(=O)C2=O]

In some embodiments, LBM is

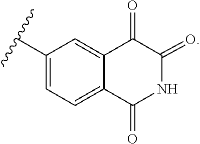 [SMILES: *c1ccc2c(c1)C(=O)C(=O)NC2=O]

In some embodiments, LBM is

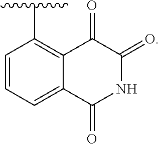 [SMILES: *c1cccc2c1C(=O)C(=O)NC2=O]

In some embodiments, LBM is selected from those in Table 1B, below.

In certain embodiments, the present invention provides a compound of formula I, wherein LBM is a RPN13 binding moiety thereby forming a compound of formula I-rrr:

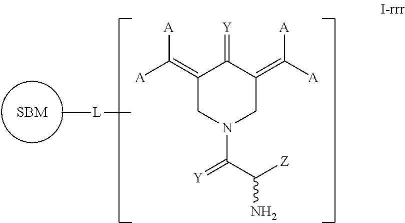

or a pharmaceutically acceptable salt thereof, wherein L and SBM are as defined above and described in embodiments herein, and wherein:
in each pair of A's, one A is hydrogen, and the other A is one of:
(i) phenyl, optionally substituted with 1-5 substituents selected from the group consisting of R¹, OR¹, NR¹R², S(O), R¹, SO₂R¹R², NR¹SO₂R², C(O)R¹, C(O)OR¹, C(O)NR¹R², NR¹C(O)R², NR¹C(O)OR², CF₃, and OCF₃;
(ii) naphthyl, optionally substituted with 1-5 substituents selected from the group consisting of R¹, OR¹, NR¹R², S(O), R¹, SO₂R¹R², NR¹SO₂R², C(O)R¹, C(O)OR¹, C(O)NR¹R, NR¹C(O)R², NR¹C(O)OR₂, CF₃, and OCF₃;
(iii) a 5 or 6 membered monocyclic heteroaryl group, having 1-3 heteroatoms selected from the group consisting of 0, N, and S, optionally substituted with 1-3 substituents selected from the group consisting of R¹, OR¹, NR₁R═, S(O), R¹, SO₂R¹R², NR¹SO₂R², C(O)R¹, C(O)OR¹, C(O)NR¹R², NR¹C(O)R², NR¹C(O)OR², CF₃, and OCF₃; and
(iv) an 8 to 10 membered bicyclic heteroalkyl group containing 1-3 heteroatoms selected from the group consisting of 0, N, and S; and the second ring is fused to the first ring using 3 to 4 carbon atoms, and the bicyclic hetero aryl group is optionally substituted with 1-3 substituents selected from the group consisting of R¹, OR¹, NR¹R². SO₂R¹R², NR¹SO₂R², C(O)R¹, C(O)OR¹, C(O)NR¹R², NR¹C(O)R², NR¹C(O)OR², CF₃, and OCF₃:
wherein Y is selected from the group consisting of O, S, NR¹ and CR¹R²;
wherein R¹ and R² are selected from the group consisting of hydrogen, nitro, hydroxyl, carboxy, amino, halogen, cyano and C₁-C₁₄ linear or branched alkyl groups, that are optionally substituted with 1-3 substituents selected from the group consisting of C₁-C₁₄ linear or branched alkyl, up to perhalo substituted C₁-C₁₄ linear or branched alkyl, C₁-C₁₄ alkoxy, hydrogen, nitro, hydroxyl, carboxy, amino, C₁-C₁₄ alkylamino, C₁-C₁₄ dialkylamino, halogen, and cyano; and
wherein Z is selected from the group consisting of hydrogen; C₁-C₁₄ linear, branched, or cyclic alkyls; phenyl; benzyl, 1-5 substituted benzyl, C₁ to C₃ alkyl-phenyl, wherein the alkyl moiety is optionally substituted with halogen up to perhalo; up to perhalo substituted C₁ to C₁₄ linear or branched alkyls; —(CH₂)_{q}—K, where K is a 5 or 6 membered monocyclic heterocyclic ring, containing 1 to 4 atoms selected from oxygen, nitrogen and sulfur, which is saturated, partially saturated, or aromatic, or an 8 to 10 membered bicyclic heteroaryl having 1-4 heteroatoms selected from the group consisting of 0, N, and S, wherein said alkyl moiety is optionally substituted with halogen up to perhalo, and wherein the variable q is an integer ranging from 0 to 4,
as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem, 2019, doi: 10.1074/jbc.AC119.010790, the entirety of each of which is herein incorporated by reference, thereby forming a compound of formula I-sss-1 or I-sss-2:

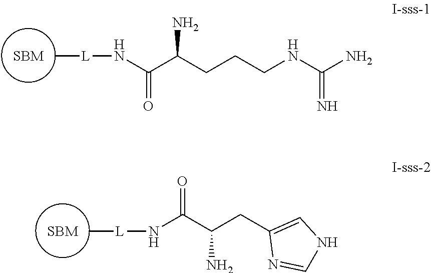

or a pharmaceutically acceptable salt thereof, wherein L and SBM are as defined above and described in embodiments herein.

In certain embodiments, the present invention provides a compound of formula I, wherein LBM is a CRBN E3 ubiquitin ligase binding moiety thereby forming a compound of formula I-uuu-1, I-uuu-2, I-uuu-3 or I-uuu-4:

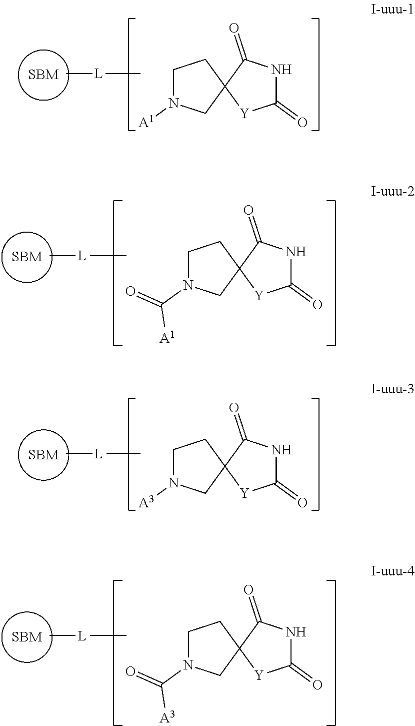

or a pharmaceutically acceptable salt thereof, wherein L and SBM are as defined above and described in embodiments herein, and wherein:
Y is NH or CH₂:
A¹ is selected from the group consisting of aryl and aryl substituted with R¹;
A³ is selected from the group consisting of heteroaryl and heteroaryl substituted with R²;
R¹ is selected from the group consisting of; —C(═O)—O—C₁₋₆-alkyl, —COOH, —NH—(C═O)—C₁₋₆-alkyl, —NH₂, and —NO₂;
R² is selected from the group consisting of —COOH, —C(═O)—O—C₁₋₆-alkyl, —NH₂, and —NO₂,
as described and defined in WO 2019/236483, the entirety of each of which is herein incorporated by reference.

In certain embodiments, the present invention provides a compound of formula I, wherein LBM is human kelch-like ECH-associated protein 1 (KEAP1) thereby forming a compound of formula I-vvv:

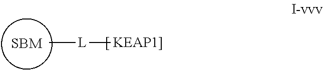

or a pharmaceutically acceptable salt thereof, wherein L and SBM are as defined above and described in embodiments herein, both singly and in combination.

In certain embodiments, the present invention provides a compound of formula I, wherein LBM is KEAP1 binding moiety as recited in Lu et al., Euro. J. Med. Chem., 2018, 146:251-9, thereby forming a compound of formula I-www:

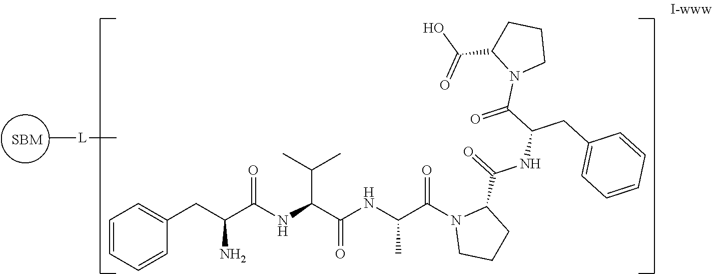

or a pharmaceutically acceptable salt thereof, wherein L and SBM are as defined above and described in embodiments herein, both singly and in combination.

In certain embodiments, the present invention provides a compound of formula I, wherein LBM is KEAP1-NRF2 binding moiety thereby forming a compound of formula I-xxx or I-xxx-2:

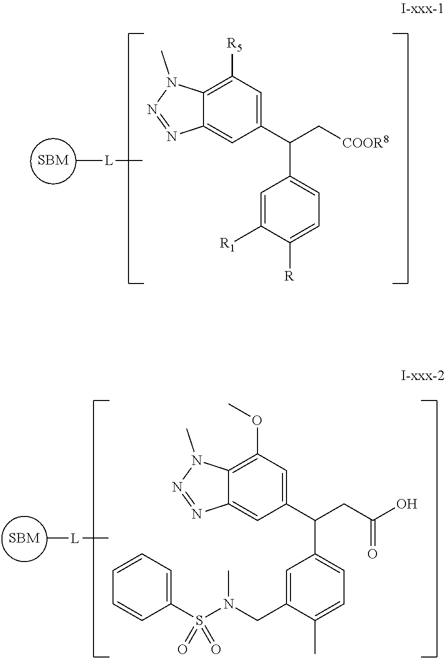

or a pharmaceutically acceptable salt thereof, wherein L and SBM are as defined above and described in embodiments herein, and wherein:
R is methyl or halo;
R¹ is

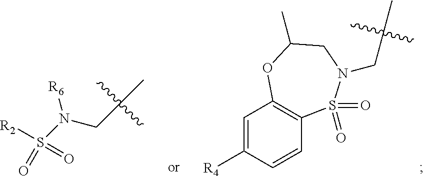

R² is methyl, or

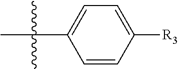

R³ is H;
R⁴ is H or halo;
R⁵ is methoxy or H;
R⁶ is H or methyl;
R⁸ is H, methyl or ethyl:
as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-yyy-1 or I-yyy-2:

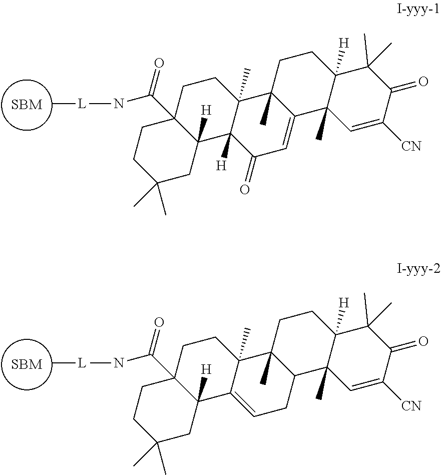

or a pharmaceutically acceptable salt thereof, wherein L and SBM are as defined above and described in embodiments herein, both singly and in combination.

In some embodiments, LBM is

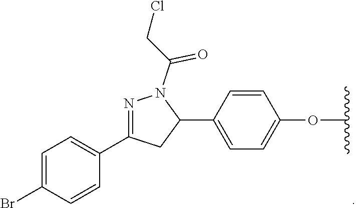 [SMILES: *Oc1ccc(C2CC(c3ccc(Br)cc3)=NN2C(=O)CCl)cc1]

In some embodiments, LBM is

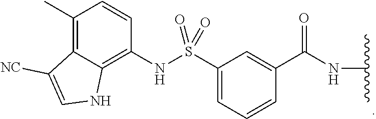 [SMILES: *NC(=O)c1cccc(S(=O)(=O)Nc2ccc(C)c3c(C#N)c[nH]c23)c1]

In some embodiments, the present disclosure provides a compound of formula I-zzz:

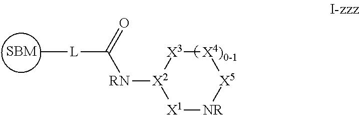

or a pharmaceutically acceptable salt thereof, wherein SBM, L, R, X¹, X², X³, X⁴, and X⁵ are as defined above and described herein.

In some embodiments, the present disclosure provides a compound of formula I-aaaa:

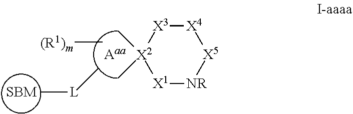

or a pharmaceutically acceptable salt thereof, wherein SBM, L, R, R¹, m, X¹, X², X³, X⁴, and X⁵ are as defined above and described herein; and:
Ring A^{aa} is a 3- to 10-membered saturated or partially unsaturated carbocyclyl or heterocyclyl ring having 1-4 heteroatoms independently selected from nitrogen, oxygen, or sulfur.

As defined above and described herein, Ring A^{aa} is a 3- to 10-membered saturated or partially unsaturated carbocyclyl or heterocyclyl ring having 1-4 heteroatoms independently selected from nitrogen, oxygen, or sulfur. In some embodiments, Ring A^{a} is a 3- to 10-membered saturated or partially unsaturated carbocyclyl. In some embodiments, Ring A^{aa} is a 3- to 10-membered saturated or partially unsaturated heterocyclyl ring having 1-4 heteroatoms independently selected from nitrogen, oxygen, or sulfur. In some embodiments, Ring A^{a} is a 5- to 6-membered saturated or partially unsaturated carbocyclyl. In some embodiments, Ring A^{aa} is a 5- to 6-membered saturated or partially unsaturated heterocyclyl ring having 1-4 heteroatoms independently selected from nitrogen, oxygen, or sulfur.

In some embodiments, Ring A^{aa} is a 5-membered saturated or partially unsaturated heterocyclyl ring having 1-4 heteroatoms independently selected from nitrogen, oxygen, or sulfur. In some embodiments, Ring A^{aa} is pyrrolidinyl. In some embodiments, Ring A^{aa} is 4,5-dihydrothiazolyl. In some embodiments, Ring A^{aa} is 4,5-dihydroisoxazolyl.

In some embodiments, a compound of formulae I-aaaa is of formula I-aaaa-1, I-aaaa-2, or I-aaaa-3:

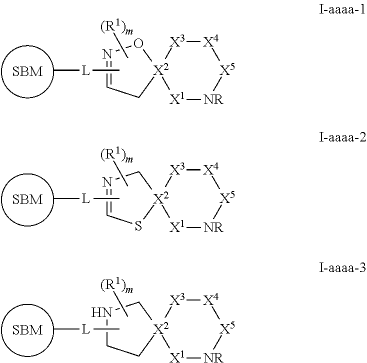

or a pharmaceutically acceptable salt thereof, wherein SBM, L, R, R¹, n, X¹, X², X³, X⁴, and X⁵ are as defined above and described herein.

In certain embodiments, the present invention provides a compound of formula I-b:

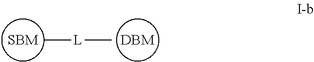

or a pharmaceutically acceptable salt thereof, wherein, SBM and L are as defined above and described herein; and
DBM is DCAF E3 ubiquitin ligase binding moiety capable of binding to DCAF1 protein.

As described above, in certain embodiments, the present invention provides a compound of formula I-b, wherein DBM is a compound of formula I-b-a or I-b-b:

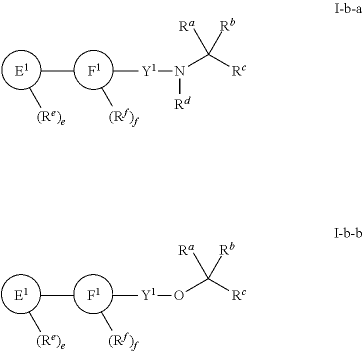

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.
Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula I-b-a where R^{d} is absent;
R^{a} is hydrogen, an optionally substituted C₁₋₆ aliphatic, or

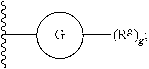

Ring G is phenyl, a 5-7 membered saturated or partially unsaturated carbocyclyl or heterocycyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R^{b} is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CRS(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur; a 5-9 membered monocyclic or bicyclic heteroaryl with 1-4 heteroatoms independently selected from nitrogen, oxygen and sulfur; or:
   —(CR₂)₂—X^{a}, wherein X^{a} is halogen or an optionally substituted ring selected from phenyl: a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or:
   R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R^{b} and R^{o} are taken together with their intervening atoms to form an optionally substituted phenyl; or:
   when Y¹ is —C(OR)═, R^{c} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R¹ is —CR₂CONR₂, R^{d} is taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂. —NRP(O)(OR)NR₂, —NRP(O)(NR₂)₂, —P(O)R₂, —P(O)(OR)₂, —P(O)(OR)NR₂, and —P(O)(NR₂)₂:
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

As described above, in certain embodiments, the present invention provides a compound of formula I-b, wherein DBM is a compound of formula I-b-c:

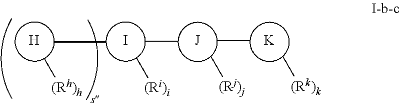

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl or a 5-10 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂. —NRP(O)(NR₂)₂, —P(O)R₂, —P(O)(OR)₂, —P(O)(OR)NR₂, and —P(O)(NR₂)₂, or:
   an R¹ group on Ring I and an R^{J} group or Ring J are optionally taken together with their intervening atoms to form a 5-8 membered saturated, partially unsaturated, or aromatic ring having 0-3 heteroatoms independently selected from nitrogen, oxygen, and sulfur, or:
each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

In certain embodiments, the present invention provides a compound of formula I-b-c as any one of the following formula:

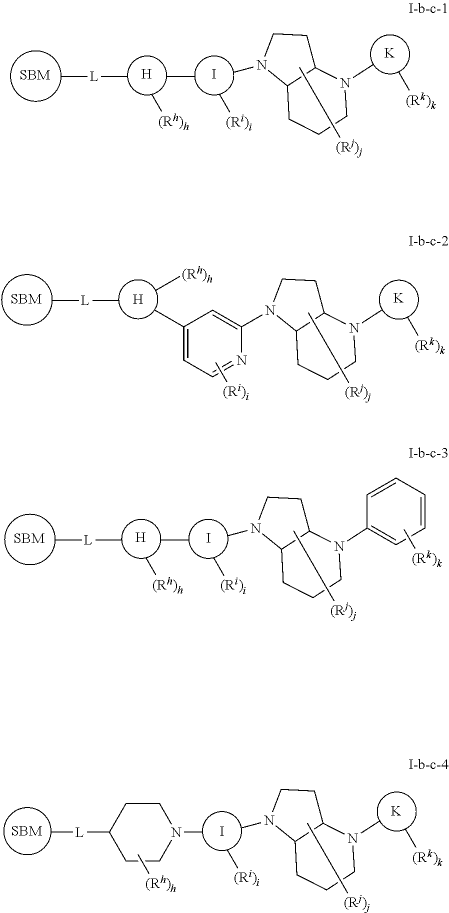

or a pharmaceutically acceptable salt thereof, wherein each of the variables is as defined above in formula I-b-c and described in embodiments herein, both singly and in combination.

In certain embodiments, the present invention provides a compound of formula I-c-a-1 or I-c-a-2 as any one of the following formula:

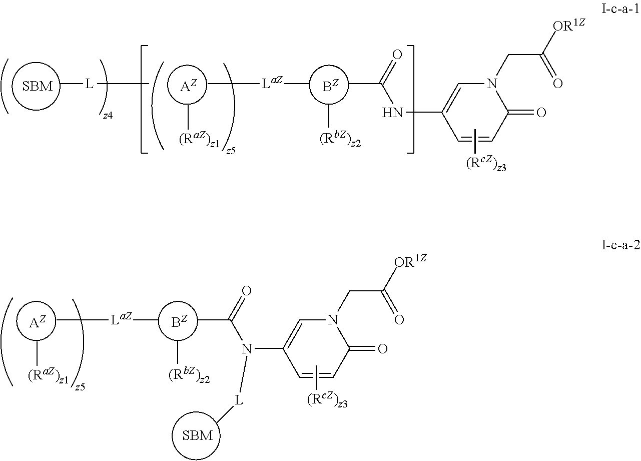

or a pharmaceutically acceptable salt thereof, wherein:
SBM and L are as defined and described above and herein;
R^{1Z} is hydrogen or optionally substituted C₁₋₆ aliphatic;
each R^{aZ}, R^{bZ}, and R^{cZ} are independently hydrogen, R^{AZ}, halogen, —CN, —NO₂, —OR^{Z}—SR^{Z}—NR^{Z}₂, —S(O)₂R^{Z}, —S(O)₂NR^{Z}₂, —S(O)R^{Z}—S(O)(NR^{Z})R^{Z}, —P(O)(OR^{Z})₂, —P(O)(NR^{Z}₂)₂, —CFR^{Z}₂, —CR^{Z}F₂, —CF₃. —CR^{Z}₂(OR^{Z}), —CR₂(NR₂), —C(O)Rz, —C(O)OR^{Z}, or —C(O)NR^{Z}₂;
each R^{AZ} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{Z} is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{Z} groups on the same atom are optionally taken together with their intervening atom to form an optionally substituted 4-11 membered saturated or partially unsaturated carbocyclic or
heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
each Ring A^{Z} is independently a bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each Ring B^{Z} is independently a bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
L^{aZ} is absent, a covalent bond, or a C₁₋₃ bivalent straight or branched saturated or unsaturated hydrocarbon chain, wherein 1-3 methylene units of the chain are independently and optionally replaced with -0-, —C(O)—, —C(S)—, —C(R^{Z})₂—, —CH(R^{Z})—, —CF(R^{Z})—, —C(F)₂, —N(Rz)-, —S—, —S(O)₂— or —CR^{Z}═CR^{Z}—:
z1, z2, and z3 are each independently 0, 1, 2, 3 or 4;
each of z4 and z5 is independently 0 or 1.

In some embodiments, a provided compound is of formula I-c-a-1, or a pharmaceutically acceptable salt thereof. In some embodiments, a provided compound is of formula I-c-a-2, or a pharmaceutically acceptable salt thereof.

In some embodiments, R^{1Z} is hydrogen, methyl, or ethyl. In some embodiments, R^{1Z} is hydrogen.

In some embodiments, each Ring A^{Z} is independently a bivalent ring selected from phenylenyl, naphthalenyl, or a 5-10 membered monocyclic or bicyclic heteroarylenyl having 1-4 heteroatoms independently selected from nitrogen, oxygen, and sulfur. In some embodiments, each Ring B is independently a bivalent ring selected from phenylenyl, a 5-6 membered saturated or partially unsaturated monocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In certain embodiments, the present invention provides a compound of formula I, wherein LBM is a CRBN E3 ubiquitin ligase binding moiety thereby forming a compound of formula I-bbbb-1:

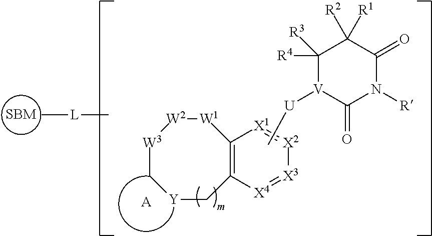

or a pharmaceutically acceptable salt thereof, wherein L and SBM are as defined above and described in embodiments herein, and
each additional variable as described and defined in CN 118005655 A, the entirety of which is herein incorporated by reference.

In certain embodiments, the present invention provides a compound of formula I, wherein LBM is a CRBN E3 ubiquitin ligase binding moiety thereby forming a compound of formula I-cccc-1:

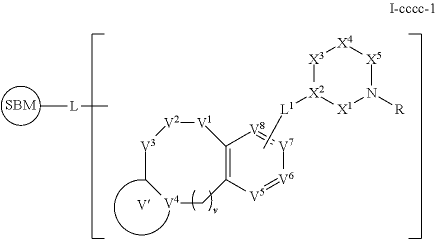

or a pharmaceutically acceptable salt thereof, wherein L, SBM, X¹, X², X³, X⁴, X⁵, R, and L¹ are as defined above and described in embodiments herein, wherein:
One of V⁵, V⁶, V⁷, and V⁵ is a carbon atom which is attached to L¹, and the others are independently selected from N or CR^{V};
each R^{V} is independently selected from hydrogen, halogen, —OR, —NR₂, —CN, or an optionally substituted group selected from C₁₋₆ aliphatic, a 3-8 membered saturated or partially unsaturated carbocyclyl or heterocyclyl having 1-3 heteroatom independently selected from nitrogen, oxygen, or sulfur, phenyl, or a 5-6 membered heteroaryl having 1-3 heteroatom independently selected from nitrogen, oxygen, or sulfur;
each of V¹, V², and V³ is independently selected form a bond, —S—, —S(O)—, —S(O)₂—, —C(O)—, —C(R^{V})—, —(C(R^{V}₂))₂—, or —N(R^{V})—,
V⁴ is selected from N, C, or CR^{V}; and
Ring V′ is an optionally substituted fused saturated or partially unsaturated carbocyclyl or heterocyclyl having 1-3 heteroatom independently selected from nitrogen, oxygen, or sulfur.

### Degradation Inducing Moiety (DIM)

In certain embodiments, the present invention provides a compound of formula I:

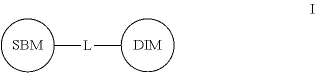

or a pharmaceutically acceptable salt thereof, wherein L and SBM are as described above and herein, and DIM is a degradation inducing moiety selected from LBM, a lysine mimetic, or a hydrogen atom.

In some embodiments, DIM is LBM as described above and herein. In some embodiments, DIM is a lysine mimetic. In some embodiments, the covalent attachment of ubiquitin to STAT6 protein is achieved through the action of a lysine mimetic. In some embodiments, upon the binding of a compound of formula I to STAT6 protein, the moiety that mimics a lysine undergoes ubiquitination thereby marking STAT6 protein for degradation via the Ubiquitin-Proteasome Pathway (UPP).

In some embodiments, DIM is

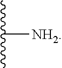 [SMILES: *N]

In some embodiments, DIM is

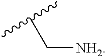 [SMILES: *CN]

In some embodiments, DIM is

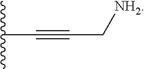 [SMILES: *C#CCN]

In some embodiments, DIM is selected from those depicted in Table 2, below.

In some embodiments, the present invention provides the compound of formula I as a compound of formula I-aaaa:

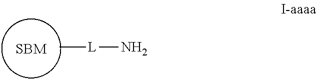

or a pharmaceutically acceptable salt thereof, wherein each of SBM and L is as defined above and described in embodiments herein, both singly and in combination.

In some embodiments, the present invention provides the compound of formula I as a compound of formula I-aaaa-1:

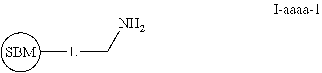

or a pharmaceutically acceptable salt thereof, wherein each of SBM and L is as defined above and described in embodiments herein, both singly and in combination.

In some embodiments, the present invention provides the compound of formula I as a compound of formula I-aaaa-2:

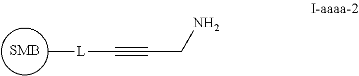

or a pharmaceutically acceptable salt thereof, wherein each of SBM and L is as defined above and described in embodiments herein, both singly and in combination.

In certain embodiments, the present invention provides a compound of Formula I, wherein DIM is a lysine mimetic

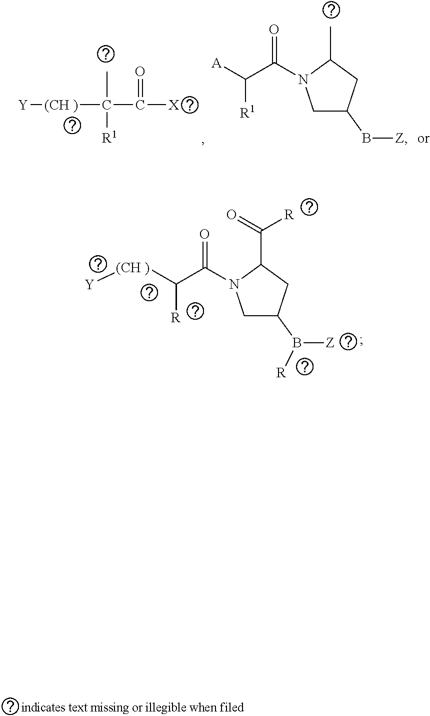

thereby forming a compound of Formulae I-bbbb-1, I-bbbb-2, or I-bbbb-3, respectively:

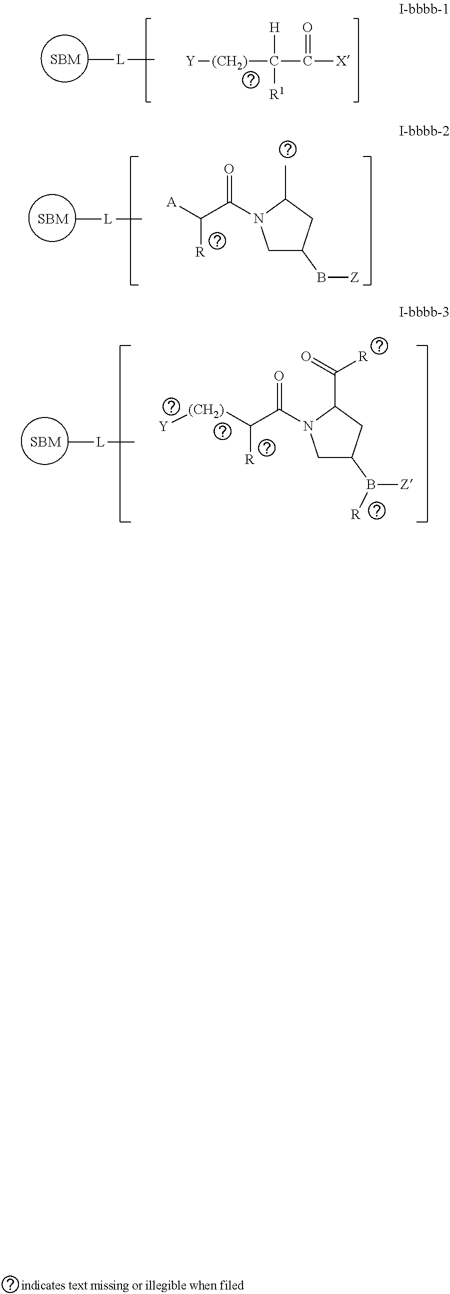

or a pharmaceutically acceptable salt thereof, wherein L and SBM are as defined above and described in embodiments herein, and wherein:
A is (CH₂)₁—Y;
k is 0, 1, or 2;
Y′ is OR² or NR²R³;
R¹ is selected from H, an optionally substituted C₁₋₁₀alkyl, an optionally substituted C₆₋₂₀aryl, an optionally substituted C₇₋₂₀aralkyl, and an amino acid side chain;
alternatively, A and R¹ together with the carbon atom to which they are bound form a 5-20 membered heteroaryl containing 1-4 ring heteroatoms independently selected from N, O, and S and optionally substituted with 1-5 Q groups;
B is selected from NR¹, NR(CH₂)ₙC(O), NR⁵(CH₂)ₙ, S(O)₂, and an amide bioisostere:
n is 0, 1, or 2;
Z is selected from H, (CH₂)ₘ—C₆₋₂₀ aryl optionally substituted with 1-5 Q groups, and (CH₂)ₘ-5-20 membered heteroaryl optionally substituted with 1-5 Q groups;
Z′ is selected from H, (CH₂)ₘ—C₆₋₂₀ aryl, (CH₂)ₘ-5-20 membered heteroaryl, C(O)(CH₂)ₘ—C₆₋₂₀ aryl, C(O)(CH₂)ₘ-5-20 membered heteroaryl, (CH₂)ₘC(O)—C₆₋₂₀ aryl, (CH₂)ₘC(O)-5-20 membered heteroaryl, S(O)₂(CH₂)ₘ—C₆₋₂₀ aryl, and S(O)₂(CH₂)ₘ-5-20 membered heteroaryl, wherein each of the C₆₋₂₀ aryl and 5-20 membered heteroaryl is optionally substituted with 1-5 Q groups;
m is 0, 1, or 2;
E is selected from C(O)OR⁶, C(O)NR⁶R⁷, a carboxylic acid bioisostere and an amide bioisostere;
Q, at each occurrence, independently is selected from an optionally substituted C₁₋₁₀alkyl, an optionally substituted C₂₋₁₀ alkenyl, an optionally substituted C₂₋₁₀alkynyl, an optionally substituted C3-20 cycloalkyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, F, Cl, Br, I, CN, CF₃, OCF₃, NO₂, OR⁸, SR, S⁺R⁸₂, S(O)R⁸, S(O)₂R⁸, S(O)₂OH, S(O)₂NR⁸R⁹, NR^{B}S(O)₂R⁹, C(O)R, C(O)OR %, C(O)NR⁸R⁹, OC(O)R, NR⁸R⁹, NRC(O)R⁹, NRC(O)OR₉, NR⁸C(O)NR⁸R⁹, and N⁺R⁸₃;
R² and R³ each independently is selected from H, an optionally substituted C₁₋₁₀alkyl, an optionally substituted C₃₋₂₀ cycloalkyl, an optionally substituted C₇-20 aralkyl, an optionally substituted C₆₋₂₀ aryl, an optionally substituted 3-20 membered cycloheteroalkyl, an optionally substituted 5-20 membered heteroaryl, C(O)R⁶, C(O)OR⁶, C(O)NR⁶R⁷, S(O)₂R⁶, and S(O)₂NR⁶R⁷;
alternatively, R² and R³ together with the nitrogen atom to which they are bound form a 3-20 membered heterocycle optionally containing 1-4 ring heteroatoms independently selected from O, N and S atoms and optionally substituted with 1-5 Q groups:
R⁵ is H or an optionally substituted C₁₋₁₀ alkyl:
R⁶ and R⁷ each independently is selected from H, an optionally substituted C₁₋₁₀ alkyl, an optionally substituted C₃₋₂₀ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, C(O)R⁸, C(O)OR^{B}, and C(O)NR⁸R⁹;
alternatively, R⁶ and R₇ together with the nitrogen atom to which they are bound form a 3-20 membered heterocycle optionally containing 1-4 ring heteroatoms independently selected from O, N and S and optionally substituted with 1-5 Q groups; and
R⁸ and R⁹ each independently is selected from H, an optionally substituted C, alkyl, an optionally substituted C³⁻²⁰ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, and an optionally substituted 5-20 membered heteroaryl, provided that the compound is not 1-(2-aminopropanoyl)-4-benzamidopyrrolidine-2-carboxylic acid.
as defined and described in U.S. Pat. No. 7,622,496, the entirety of each of which is herein incorporated by reference.

### Linker (L)

As defined above and described herein, L is a bivalent moiety that connects SBM to LBM or SBM to DIM.

In some embodiments, L is a bivalent moiety that connects SBM to LBM. In some embodiments, L is a bivalent moiety that connects SBM to DIM. In some embodiments, L is a bivalent moiety that connects SBM to a lysine mimetic.

In some embodiments, L is a covalent bond or an optionally substituted bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

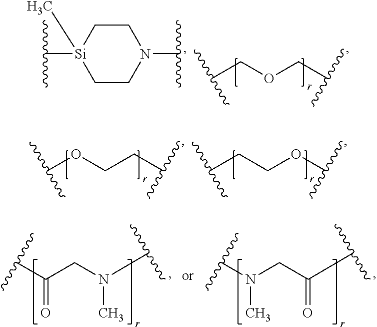

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, C(O)NR—, —OC(O)NR—, —NRC(O)O—,

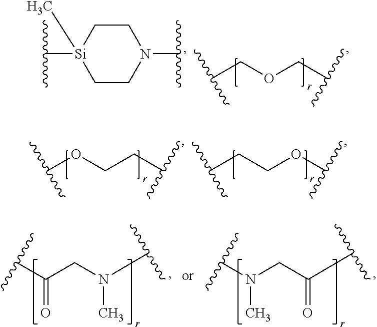

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or a bivalent, saturated or partially unsaturated, straight or branched C₁₋₅₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —C(O)NR—, —OC(O)NR—, —NRC(O)O—,

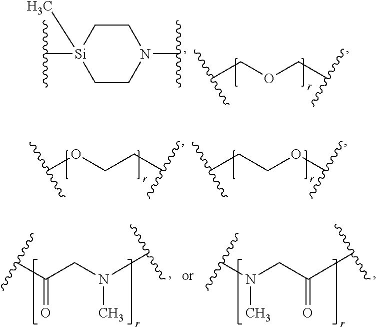

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋ₒ hydrocarbon chain, wherein 0-4 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

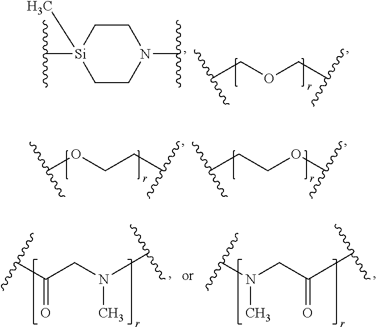

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond. In some embodiments, L is a bivalent, saturated or partially unsaturated, straight or branched C₁₋₅₀, C₁₋₄₀, C₁₋₃₀, C₁₋₂₀, or C₁₋₁₀ hydrocarbon chain, wherein 0-6 methylene units of L are independently replaced by -Cy-. —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

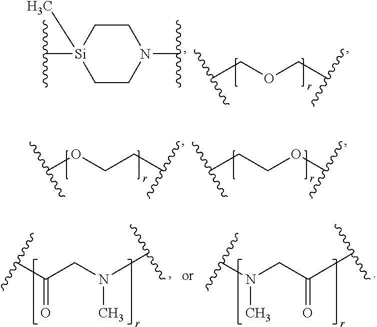

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, —NRC(O)O—,

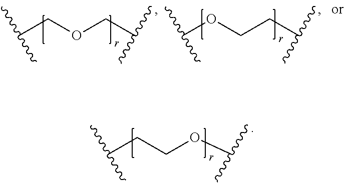

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

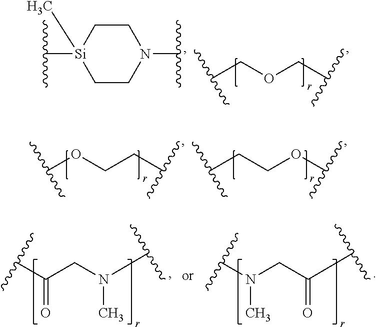

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₁₀ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃; hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

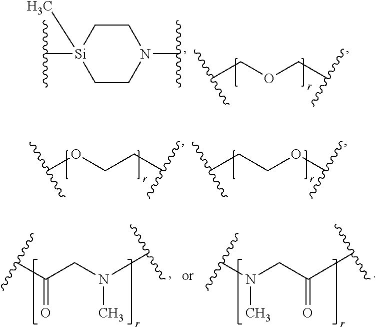

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR— or —NRC(O)O.

In some embodiments, L is an optionally substituted bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

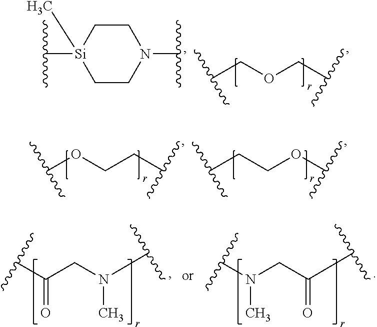

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₃ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

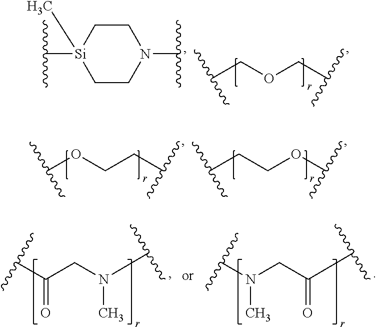

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{o}. In some embodiments, L is an optionally substituted bivalent, saturated or partially unsaturated, straight or branched C₁₋₂ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, or NRC(O)O—, provided that L does not comprise —NR—C(O)—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —S—, —C(O)O—, —C(O)—, —S(O)—, —S(O)₂—, —NRS(O)₂—, —NRC(O)—, or NRC(O)O—.

In some embodiments, L is —NR—(C₁₋₁₀ aliphatic)-. In some embodiments, L is —(C₁₋₁₀ aliphatic)-NR—(C₁₋₁₀ aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ (aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is —(C₁₋₁₀ aliphatic)-. In some embodiments, L is -Cy-(C₁₋₁₀ aliphatic)-. In some embodiments, L is —(C₁₋₁₀ aliphatic)-Cy-(C₁₋₁₀ o aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is -Cy-(optionally substituted C₁₋₁₀ aliphatic)-. In some embodiments, L is -Cy-(optionally substituted C₁₋₁₀ aliphatic)-Cy-. In some embodiments, L is -Cy-Cy-(optionally substituted C₁₋₁₀ aliphatic)-. In some embodiments, L is -Cy-Cy-Cy-(optionally substituted C₁₋₁₀ aliphatic)-. In some embodiments, L is -Cy-Cy-(optionally substituted C₁₋₁₀ aliphatic)-Cy-. In some embodiments, L is -Cy-(C₁₋₁₀ aliphatic)-. In some embodiments, L is -Cy-Cy-(C₁₋₁₀ aliphatic)-. In some embodiments, L is -Cy-Cy-Cy-(C₁₋₁₀ aliphatic)-. In some embodiments, L is -Cy-Cy-(C₁₋₁₀ aliphatic)-Cy-.

In some embodiments, L is -Cy-(optionally substituted C₁₋₆ aliphatic)-. In some embodiments, L is -Cy-(optionally substituted C₁₋₆ aliphatic)-Cy-. In some embodiments, L is -Cy-Cy-(optionally substituted C₁₋₆ aliphatic)-. In some embodiments, L is -Cy-Cy-Cy-(optionally substituted C₁₋₆ aliphatic)-. In some embodiments, L is -Cy-Cy-(optionally substituted C₁₋₆ aliphatic)-Cy-. In some embodiments, L is -Cy-(C₁₋₆ aliphatic)-. In some embodiments, L is -Cy-(C₁₋₆ aliphatic)-Cy-. In some embodiments, L is -Cy-Cy-(C₁₋₆ aliphatic)-. In some embodiments, L is -Cy-Cy-Cy-(C₁₋₆ aliphatic)-. In some embodiments, L is -Cy-Cy-(C₁₋₆ aliphatic)-Cy-.

In some embodiments, L is -Cy-(optionally substituted C₁₋₃ aliphatic)-. In some embodiments, L is -Cy-(optionally substituted C₁₋₃ aliphatic)-Cy-. In some embodiments, L is -Cy-Cy-(optionally substituted C₁₋₃ aliphatic)-. In some embodiments, L is -Cy-Cy-Cy-(optionally substituted C₁₋₃ aliphatic)-. In some embodiments, L is -Cy-Cy-(optionally substituted C₁₋₃ aliphatic)-Cy-. In some embodiments, L is -Cy-(C₁₋₃ aliphatic)-. In some embodiments, L is -Cy-(C₁₋₃ aliphatic)-Cy-. In some embodiments, L is -Cy-Cy-(C₁₋₃ aliphatic)-. In some embodiments, L is -Cy-Cy-Cy-(C₁₋₃ aliphatic)-. In some embodiments, L is -Cy-Cy-(C₁₋₃ aliphatic)-Cy-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)-o-NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)-co-.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀—, -Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)-o-. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—. In some embodiments, L is -Cy-Cy-. In some embodiments, L is -Cy-Cy-(CH₂)₁₋₁₀—. In some embodiments, L is -Cy-(CH₂)₁₋₁₀-Cy-(CH₂)₁₋₁₀—. In some embodiments, L is -Cy-Cy-Cy-. In some embodiments, L is -Cy-Cy-(CH₂)₁₋₁₀-Cy-. In some embodiments, L is -Cy-Cy-(CH₂)₁₋₁₀-Cy-(CH₂)₁₋₁₀—.

In some embodiments, L is optionally substituted -Cy-(CH₂)₁₋₁₀—. In some embodiments, L is optionally substituted —(CH₂)₁₋₁₀-Cy-(CH₂)₁₋₁₀—. In some embodiments, L is optionally substituted —(CH₂)₁₋₁₀-Cy-(CH₂CH₂O)₁₋₁₀CH₂CH₂—. In some embodiments, L is optionally substituted -Cy-(CH₂)₁₋₁₀-Cy-. In some embodiments, L is optionally substituted -Cy-(CH₂)₁₋₁₀-Cy-(CH₂)₁₋₁₀—. In some embodiments, L is optionally substituted -Cy-(CH₂)₁₋₁₀-Cy-(CH₂)₁₋₁₀-Cy-. In some embodiments, L is optionally substituted —(CH₂)₁₋₁₀-Cy-(CH₂)₁₋₁₀-Cy-(CH₂)₁₋₁₀—. In some embodiments, L is optionally substituted -Cy-Cy-. In some embodiments, L is optionally substituted -Cy-Cy-(CH₂)₁₋₁₀—. In some embodiments, L is optionally substituted -Cy-(CH₂)₁₋₁₀-Cy-(CH₂)₁₋₁₀—. In some embodiments, L is optionally substituted -Cy-Cy-Cy-. In some embodiments, L is optionally substituted -Cy-Cy-(CH₂)₁₋₁₀-Cy-. In some embodiments, L is optionally substituted -Cy-Cy-(CH₂)₁₋₁₀-Cy-(CH₂)₁₋₁₀—.

In some embodiments, L comprises one -Cy- group. In some embodiments, L comprises two -Cy- groups. In some embodiments, L comprises three -Cy- groups. In some embodiments, L does not comprise a -Cy- group.

In some embodiments, L comprises —C(O)—.

In some embodiments, L comprises one -Cy- group and one methylene group. In some embodiments, L comprises two -Cy- groups and one methylene group. In some embodiments, L comprises one -Cy- group and two methylene groups.

In some embodiments, L is an optionally substituted bivalent, saturated or partially unsaturated, straight or branched C₁₋ₒ hydrocarbon chain, wherein 1-4 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋ₒ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 3 methylene units of L are independently replaced by -Cy-, and 1-2 additional methylene units of L are optionally and independently replaced with —CHF—, —CF2-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched Cis hydrocarbon chain, wherein 3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₃ hydrocarbon chain, wherein 2 methylene units of L are independently replaced by -Cy-, and 1 additional methylene unit of L is optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 3 methylene units of L are independently replaced by -Cy-. In some embodiments, L is an optionally substituted bivalent, saturated or partially unsaturated, straight or branched C₁₋₃ hydrocarbon chain, wherein 1 methylene unit of L is replaced by —C(O)—, and 1-2 additional methylene units of L are optionally and independently replaced with -Cy-, —CHF—, —CF2-, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is:

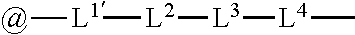

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-, Cy^{L2}-, -Cy^{L3}-, and -Cy^{L4}- is independently -Cy-, wherein -Cy- is as defined above and described herein.

In some embodiments, L is @-L¹-L²-L³-. In some embodiments, L is @-L¹-L²-. In some embodiments, L is @-L¹-.

In some embodiments, L¹ is a covalent bond or an optionally substituted bivalent, saturated or partially unsaturated, straight or branched C₁₋₆ hydrocarbon chain, wherein 1 methylene unit of L¹ is optionally replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L¹ is a covalent bond. In some embodiments, L¹ is an optionally substituted bivalent, saturated or partially unsaturated, straight or branched C₁₋₆ hydrocarbon chain, wherein 1-2 methylene units of L¹ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L¹ is -Cy^{L1}-. In some embodiments, L¹ is an optionally substituted bivalent, saturated or partially unsaturated, straight or branched C₁₋₆ hydrocarbon chain. In some embodiments, L¹ is an optionally substituted bivalent, saturated or partially unsaturated, straight or branched C₁₋₃ hydrocarbon chain. In some embodiments, L¹ is an optionally substituted bivalent, saturated or partially unsaturated, straight or branched C₁₋₆ hydrocarbon chain, wherein 1 methylene unit of L¹ is optionally and independently replaced by —O— or —NR—. In some embodiments, L¹ is —O—. In some embodiments, L¹ is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L¹ is —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—.

In some embodiments, L² is a covalent bond or an optionally substituted bivalent, saturated or partially unsaturated, straight or branched C₁₋₆ hydrocarbon chain, wherein 1 methylene unit of L¹ is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L² is a covalent bond. In some embodiments, L² is an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L² is -Cy^{L2}-. In some embodiments, L² is an optionally substituted bivalent, saturated or partially unsaturated, straight or branched C₁₋₆ hydrocarbon chain. In some embodiments, L² is an optionally substituted bivalent, saturated or partially unsaturated, straight or branched C₁₋₃ hydrocarbon chain. In some embodiments, L¹ is —O—. In some embodiments, L² is optionally substituted —CH₂-, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L² is —CH₂—, —CH₂CH₂—, —CH═CH—, —C═C—, —CH₂CH₂CH₂—, or —CH═CHCH₂—, —CH₂CH═CH—, —C—CCH₂—, or —CH₂C═C—.

In some embodiments, L³ is a covalent bond or an optionally substituted bivalent, saturated or partially unsaturated, straight or branched C₁₋₆ hydrocarbon chain, wherein 1 methylene unit of L¹ is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L³ is a covalent bond. In some embodiments, L³ is an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L³ is -Cy^{L3}. In some embodiments, L³ is an optionally substituted bivalent, saturated or partially unsaturated, straight or branched C₁₋₆ hydrocarbon chain. In some embodiments, L³ is an optionally substituted bivalent, saturated or partially unsaturated, straight or branched C₁₋₃ hydrocarbon chain. In some embodiments, L³ is —O—. In some embodiments, L³ is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L³ is —CH₂—, —CH₂CH₂—, —CH═CH—, —C═C—, —CH₂CH₂CH₂—, or —CH═CHCH₂—, —CH₂CH═CH—, —C₂CCH₂—, or —CH₂C═C—.

In some embodiments, L⁴ is a covalent bond or an optionally substituted bivalent, saturated or partially unsaturated, straight or branched C₁₋₃ hydrocarbon chain, wherein 1 methylene unit of L^{w} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L⁴ is a covalent bond. In some embodiments, L⁴ is an optionally substituted bivalent, saturated or partially unsaturated, straight or branched C₁₋₆ hydrocarbon chain, wherein 1-2 methylene units of L⁴ are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L⁴ is -Cy^{L1}-. In some embodiments, L⁴ is an optionally substituted bivalent, saturated or partially unsaturated, straight or branched C₁₋₆ hydrocarbon chain. In some embodiments, L⁴ is an optionally substituted bivalent, saturated or partially unsaturated, straight or branched C₁₋₃ hydrocarbon chain. In some embodiments, L⁴ is —O—. In some embodiments, L⁴ is optionally substituted —CH₂—, —CH₂CH₂—, —CH═CH—, —C═C—, —CH₂CH₂CH₂—, or —CH═CHCH₂—, —CH₂CH═CH—, —C═CCH₂—, or —CH₂C═C—. In some embodiments, L⁴ is —CH₂—, —CH₂CH₂—, —CH═CH—, —C═C—, —CH₂CH₂CH₂—, or —CH═CHCH₂—, —CH^{Z}CH═CH—, —C═CCH₂—, or —CH₂C═C—.

As defined above and described herein, each of -Cy^{L2}-, Cy^{L2}-, -Cy-, and -Cy^{L1}- is independently -Cy-, wherein -Cy- is as defined above and described herein.

In some embodiments, -Cy^{L1}- is an optionally substituted 8-10 membered bicyclic arylenyl. In some embodiments, -Cy^{L1}- is an optionally substituted 3-7 membered saturated or partially unsaturated carbocyclylenyl. In some embodiments, -Cy^{L1}- is an optionally substituted 6-11 membered saturated or partially unsaturated spiro carbocyclylenyl. In some embodiments, -Cy^{L1}- is an 8-10 membered bicyclic saturated or partially unsaturated carbocyclylenyl. In some embodiments, -Cy^{L1}- is an optionally substituted a 4-7 membered saturated or partially unsaturated heterocyclylenyl having 1-2 heteroatoms independently selected from nitrogen, oxygen, and sulfur. In some embodiments, -Cy^{L1}- is an optionally substituted 6-11 membered saturated or partially unsaturated spiro heterocyclylenyl having 1-3 heteroatoms independently selected from nitrogen, oxygen, and sulfur. In some embodiments, -Cy^{L1}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L1}- is an optionally substituted 5-6 membered heteroarylenyl having 1-4 heteroatoms independently selected from nitrogen, oxygen. In some embodiments, -Cy^{L1}- is an optionally substituted 8-10 membered bicyclic heteroarylenyl having 1-5 heteroatoms independently selected from nitrogen, oxygen, and sulfur.

In some embodiments, each -C^{L1}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an 8-10 membered bicyclic arylenyl. In some embodiments, -Cy^{L1}- is a 3-7 membered saturated or partially unsaturated carbocyclylenyl. In some embodiments, -Cy^{L1}- is a 6-11 membered saturated or partially unsaturated spiro carbocyclylenyl. In some embodiments, -Cy^{L1}- is an 8-10 membered bicyclic saturated or partially unsaturated carbocyclylenyl. In some embodiments, -Cy^{L1}- is a 4-7 membered saturated or partially unsaturated heterocyclylenyl having 1-2 heteroatoms independently selected from nitrogen, oxygen, and sulfur. In some embodiments, -Cy^{L1}- is a 6-11 membered saturated or partially unsaturated spiro heterocyclylenyl having 1-3 heteroatoms independently selected from nitrogen, oxygen, and sulfur. In some embodiments, -Cy^{L1}- is an 8-10 membered bicyclic saturated or partially unsaturated heterocyclylenyl having 1-2 heteroatoms independently selected from nitrogen, oxygen, and sulfur. In some embodiments, -Cy^{L1}- is a 5-6 membered heteroarylenyl having 1-4 heteroatoms independently selected from nitrogen, oxygen. In some embodiments, -Cy^{L1}- is an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L1}- is an optionally substituted phenylenyl. In some embodiments, -Cy^{L1}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o}is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L2}- is an optionally substituted 5-6 membered saturated or partially unsaturated heterocyclylenyl having 1-2 heteroatoms independently selected from nitrogen, oxygen, and sulfur. In some embodiments, -Cy^{L1}- is an optionally substituted 5 membered saturated or partially unsaturated heterocyclylenyl having 1-2 heteroatoms independently selected from nitrogen, oxygen, and sulfur. In some embodiments, -Cy^{L1}- is an optionally substituted pyrrolidinylenyl. In some embodiments, -Cy^{L1}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrrolidinylenyl, optionally substituted with halogen (e.g., fluoro or chloro) or C₁₋₆ aliphatic. In some embodiments, -Cy- is an optionally substituted 6 membered saturated or partially unsaturated heterocyclylenyl having 1-2 heteroatoms independently selected from nitrogen, oxygen, and sulfur. In some embodiments, -Cy^{L1}- is an optionally substituted piperadinylenyl. In some embodiments, -Cy^{L1}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is piperadinylenyl, optionally substituted with halogen (e.g., fluoro or chloro) or C₁₋₆ aliphatic. In some embodiments, -Cy^{L2}- is an optionally substituted piperazinylenyl. In some embodiments, -Cy^{L1}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is piperazinylenyl, optionally substituted with halogen (e.g., fluoro or chloro) or C₁e aliphatic.

In some embodiments, -C^{L1}- is an optionally substituted 5 membered heteroarylenyl having 1-4 heteroatoms independently selected from nitrogen, oxygen. In some embodiments, -Cy^{L1}- is an optionally substituted pyrrolylenyl, pyrazolylenyl, imidazolylenyl, or triazolylenyl. In some embodiments, -Cy^{L1}- is pyrrolylenyl, pyrazolylenyl, imidazolylenyl, or triazolylenyl, optionally substituted with halogen (e.g., fluoro or chloro), —CN, C₁e aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L1}- is an optionally substituted pyrazolylenyl. In some embodiments, -Cy^{L1}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyrazolylenyl, optionally substituted with halogen (e.g., fluoro or chloro) or C₁₋₆ aliphatic. In some embodiments, -Cy^{L1}- is an optionally substituted imidazolylenyl. In some embodiments, -Cy^{L1}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L1}- is imidazolylenyl, optionally substituted with halogen (e.g., fluoro or chloro) or C₁₋₆ aliphatic.

In some embodiments, -Cy^{L1}- is an optionally substituted 6 membered heteroarylenyl having 1-4 heteroatoms independently selected from nitrogen, oxygen. In some embodiments, -Cy^{L1}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁s aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L1}- is an optionally substituted pyridinylenyl. In some embodiments, -Cy^{L1}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinylenyl, optionally substituted with halogen (e.g., fluoro or chloro) or C₁₋₆ aliphatic. In some embodiments, -Cy^{L1}- is an optionally substituted pyridazinylenyl. In some embodiments, -Cy^{L1}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L1}- is an optionally substituted pyridinonylenyl. In some embodiments, -Cy^{L1}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, optionally substituted with halogen (e.g., fluoro or chloro) or C₆ aliphatic.

In some embodiments, -Cy^{L1}- is an optionally substituted cyclopropyl. In some embodiments, -Cy^{L1}- is an optionally substituted 5 membered saturated or partially unsaturated carbocyclylenyl. In some embodiments, -Cy^{L1}- is an optionally substituted cyclopentanylenyl or cyclopentenylenyl. In some embodiments, -Cy^{L1}- is an optionally substituted 6 membered saturated or partially unsaturated carbocyclylenyl. In some embodiments, -Cy^{L1}- is an optionally substituted cyclohexanylenyl or cyclohexenylenyl. In some embodiments, -Cy^{L1}- is an optionally substituted naphthalenylenyl.

In some embodiments, -Cy^{L1}- is an optionally substituted 9 membered bicyclic heteroarylenyl having 1-5 heteroatoms independently selected from nitrogen, oxygen, and sulfur. In some embodiments, -Cy^{L1}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L1}- is an optionally substituted indolylenyl. In some embodiments, -Cy^{L1}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L1}- is an optionally substituted 8-11 membered saturated or partially unsaturated spiro heterocyclylenyl having 1-3 heteroatoms independently selected from nitrogen, oxygen, and sulfur. In some embodiments, -Cy^{L1}- is an optionally substituted 9-11 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 nitrogen heteroatoms. In some embodiments, -Cy^{L1}- is an optionally substituted 4,6-spiro heterocyclylenyl having 1-3 heteroatoms independently selected from nitrogen, oxygen, and sulfur. In some embodiments, -Cy^{L1}- is an optionally substituted

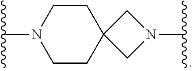 [SMILES: *N1CCC2(CC1)CN(*)C2]

In some embodiments, -Cy^{L1}- is an optionally substituted 10 membered saturated or partially unsaturated spiro heterocyclylenyl having 1-3 heteroatoms independently selected from nitrogen, oxygen, and sulfur. In some embodiments, -Cy^{L1}- is an optionally substituted 10 membered saturated or partially unsaturated spiro heterocyclylenyl having 1-3 nitrogen heteroatoms. In some embodiments, -Cy^{L2}- is an optionally substituted 5,6-spiro heterocyclylenyl having 1-3 heteroatoms independently selected from nitrogen, oxygen, and sulfur.

In some embodiments, -Cy^{L2}i- is an optionally substituted 11 membered saturated or partially unsaturated spiro heterocyclylenyl having 1-3 heteroatoms independently selected from nitrogen, oxygen, and sulfur. In some embodiments, -Cy^{L2}- is an optionally substituted 11 membered saturated or partially unsaturated spiro heterocyclylenyl having 1-3 nitrogen heteroatoms. In some embodiments, -Cy^{L2}- is an optionally substituted 6,6-spiro heterocyclylenyl having 1-3 heteroatoms independently selected from nitrogen, oxygen, and sulfur.

In some embodiments, -Cy^{L2}- is optionally substituted

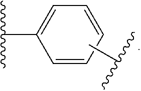

In some embodiments, -Cy^{L1}- is

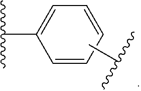

In some embodiments, -Cy^{L1}- is

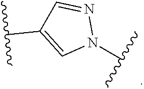 [SMILES: *c1cnn(*)c1]

In some embodiments, -Cy^{L1}- is

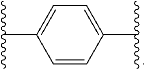 [SMILES: *c1ccc(*)cc1]

In some embodiments, -Cy^{L1}- is

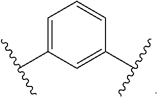 [SMILES: *c1cccc(*)c1]

In some embodiments, -Cy^{L1}- is

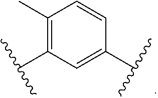 [SMILES: *c1ccc(C)c(*)c1]

In some embodiments, -Cy^{L1}- is

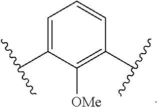 [SMILES: *c1cccc(*)c1OC]

In some embodiments, -Cy^{L1}- is

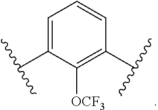 [SMILES: *c1cccc(*)c1OC(F)(F)F]

In some embodiments, -Cy^{L1}- is

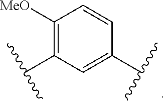 [SMILES: *c1ccc(OC)c(*)c1]

In some embodiments, -Cy^{L1}- is

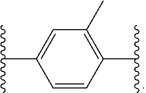 [SMILES: *c1ccc(*)c(C)c1]

In some embodiments, -Cy^{L1}- is

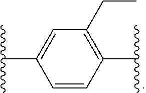 [SMILES: *c1ccc(*)c(CC)c1]

In some embodiments, -Cy^{L1}- is

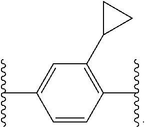 [SMILES: *c1ccc(*)c(C2CC2)c1]

In some embodiments, -Cy^{L1}- is

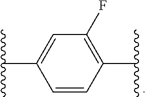 [SMILES: *c1ccc(*)c(F)c1]

In some embodiments, -Cy^{L1}- is

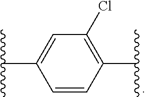 [SMILES: *c1ccc(*)c(Cl)c1]

In some embodiments, -Cy^{L1}- is

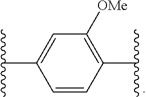 [SMILES: *c1ccc(*)c(OC)c1]

In some embodiments, -Cy^{L1}- is

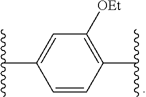 [SMILES: *c1ccc(*)c(OCC)c1]

In some embodiments, -Cy^{L1}- is

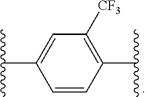 [SMILES: *c1ccc(*)c(C(F)(F)F)c1]

In some embodiments, -Cy^{L1}- is

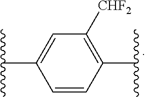 [SMILES: *c1ccc(*)c(C(F)F)c1]

In some embodiments, -Cy^{L1}- is

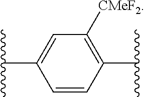

In some embodiments, -Cy^{L1}- is

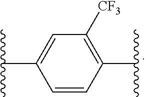 [SMILES: *c1ccc(*)c(C(F)(F)F)c1]

In some embodiments, -Cy^{L1}- is

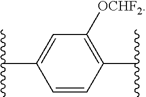 [SMILES: *c1ccc(*)c(OC(F)F)c1]

In some embodiments, -Cy^{L1}- is

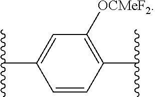

In some embodiments, -Cy^{L1}- is

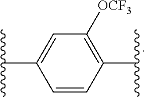 [SMILES: *c1ccc(*)c(OC(F)(F)F)c1]

In some embodiments, -Cy^{L1}- is

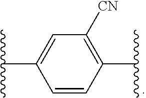 [SMILES: *c1ccc(*)c(C#N)c1]

In some embodiments, -Cy^{L1}- is

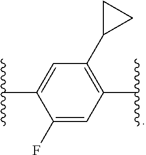 [SMILES: *c1cc(C2CC2)c(*)cc1F]

In some embodiments, -Cy^{L1}- is

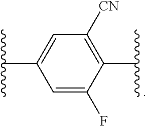 [SMILES: *c1cc(F)c(*)c(C#N)c1]

In some embodiments, -Cy^{L1}- is

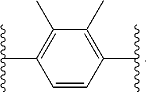 [SMILES: *c1ccc(*)c(C)c1C]

In some embodiments, -Cy^{L1}- is

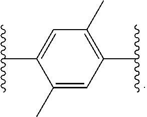 [SMILES: *c1cc(C)c(*)cc1C]

In some embodiments, -Cy^{L1}- is

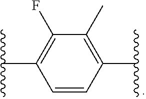 [SMILES: *c1ccc(*)c(F)c1C]

In some embodiments, -Cy^{L1}- is

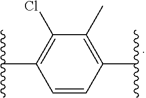 [SMILES: *c1ccc(*)c(Cl)c1C]

In some embodiments, -Cy^{L1}- is

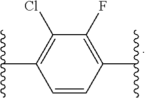 [SMILES: *c1ccc(*)c(Cl)c1F]

In some embodiments, -Cy^{L1}- is

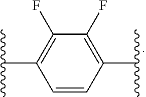 [SMILES: *c1ccc(*)c(F)c1F]

In some embodiments, -Cy^{L1}- is

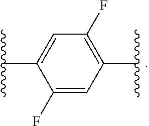 [SMILES: *c1cc(F)c(*)cc1F]

In some embodiments, -Cy^{L1}- is

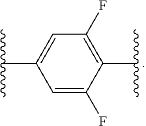 [SMILES: *c1cc(F)c(*)c(F)c1]

In some embodiments, -Cy^{L1}- is

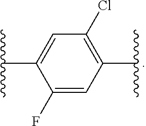 [SMILES: *c1cc(Cl)c(*)cc1F]

In some embodiments, -Cy^{L1}- is

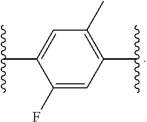 [SMILES: *c1cc(F)c(*)cc1C ; "."]

In some embodiments, -Cy^{L1}- is

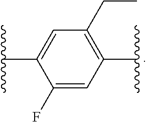 [SMILES: *c1cc(CC)c(*)cc1F ; "."]

In some embodiments, -Cy^{L1}- is

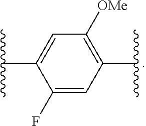 [SMILES: *c1cc(OC)c(*)cc1F]

In some embodiments, -Cy^{L1}- is

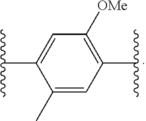 [SMILES: *c1cc(OC)c(*)cc1C]

In some embodiments, -Cy^{L1}- is

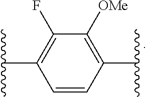 [SMILES: *c1ccc(*)c(OC)c1F ; "."]

In some embodiments, -Cy^{L1}- is

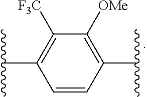 [SMILES: *c1ccc(*)c(C(F)(F)F)c1OC ; "."]

In some embodiments, -Cy^{L1}- is

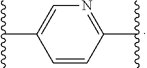 [SMILES: *c1ccc(*)nc1 ; "."]

In some embodiments, -Cy^{L1}- is

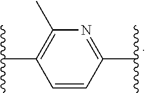 [SMILES: *c1ccc(*)c(C)n1]

In some embodiments, -Cy^{L1}- is

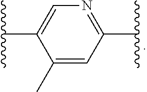 [SMILES: *c1cc(C)c(*)cn1]

In some embodiments, -Cy^{L1}- is

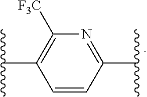 [SMILES: *c1ccc(*)c(C(F)(F)F)n1]

In some embodiments, -Cy^{L1}- is

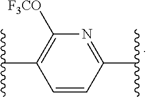 [SMILES: *c1ccc(*)c(OC(F)(F)F)n1]

In some embodiments, -Cy^{L1}- is

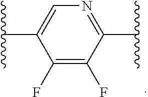 [SMILES: *c1cnc(*)c(F)c1F]

In some embodiments, -Cy^{L1}- is

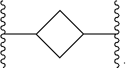 [SMILES: *C1CC(*)C1]

In some embodiments, -Cy^{L1}- is

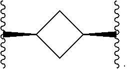 [SMILES: *[C@H]1C[C@@H](*)C1]

In some embodiments, -Cy^{L1}- is

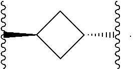 [SMILES: *[C@H]1C[C@H](*)C1]

In some embodiments, -Cy^{L1}- is

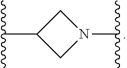 [SMILES: *C1CN(*)C1]

In some embodiments, -Cy^{L1}- is

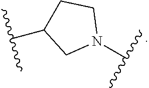 [SMILES: *C1CCN(*)C1]

In some embodiments, -Cy^{L1}- is

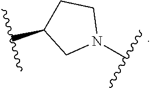 [SMILES: *[C@H]1CCN(*)C1]

In some embodiments, -Cy^{L1}- is

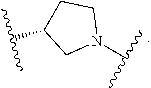 [SMILES: *[C@@H]1CCN(*)C1]

In some embodiments, -Cy^{L1}- is

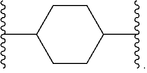 [SMILES: *C1CCC(*)CC1]

In some embodiments, -Cy^{L1}- is

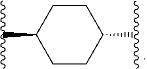 [SMILES: *[C@H]1CC[C@H](*)CC1]

In some embodiments, -Cy^{L1}- is

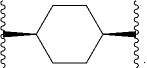 [SMILES: *[C@H]1CC[C@@H](*)CC1]

In some embodiments, -Cy^{L1}- is

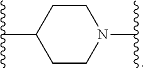 [SMILES: *C1CCN(*)CC1]

In some embodiments, -Cy^{L1}- is

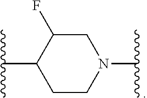 [SMILES: *C1CCN(*)CC1F ; "."]

In some embodiments, -Cy^{L1}- is

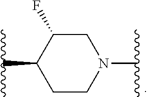 [SMILES: *[C@@H]1CCN(*)C[C@H]1F]

In some embodiments, -Cy^{L1}- is

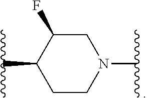 [SMILES: *[C@@H]1CCN(*)C[C@@H]1F ; "."]

In some embodiments, -Cy^{L1}- is

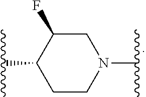 [SMILES: *[C@H]1CCN(*)C[C@@H]1F]

In some embodiments, -Cy^{L1}- is

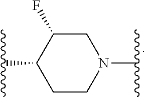 [SMILES: *[C@H]1CCN(*)C[C@H]1F]

In some embodiments, -Cy^{L1}- is

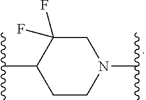 [SMILES: *C1CCN(*)CC1(F)F]

In some embodiments, -Cy^{L1}- is

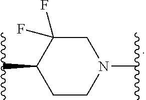 [SMILES: *[C@@H]1CCN(*)CC1(F)F]

In some embodiments, -Cy^{L1}- is

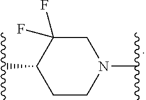 [SMILES: *[C@H]1CCN(*)CC1(F)F]

In some embodiments, -Cy^{L1}- is

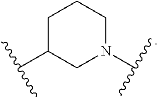 [SMILES: *C1CCCN(*)C1 ; "."]

In some embodiments, -Cy^{L1}- is

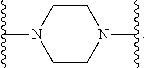 [SMILES: *N1CCN(*)CC1]

In some embodiments, -Cy^{L1}- is

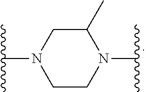 [SMILES: *N1CCN(*)C(C)C1]

In some embodiments, -Cy^{L1}- is

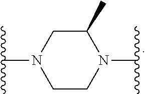 [SMILES: *N1CCN(*)[C@H](C)C1]

In some embodiments, -Cy^{L1}- is

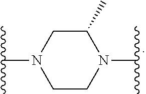 [SMILES: *N1CCN(*)[C@@H](C)C1]

In some embodiments, -Cy^{L1}- is

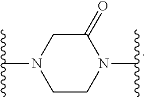 [SMILES: *N1CCN(*)C(=O)C1]

In some embodiments, -Cy^{L1}- is

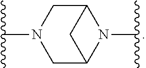 [SMILES: *N1CC2CC(C1)N2*]

In some embodiments, -Cy^{L1}- is

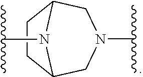 [SMILES: *N1CC2CCC(C1)N2*]

In some embodiments, -Cy^{L1}- is

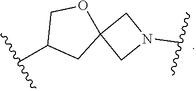 [SMILES: *C1COC2(C1)CN(*)C2]

In some embodiments, -Cy^{L1}- is

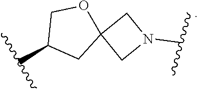 [SMILES: *[C@H]1COC2(C1)CN(*)C2]

In some embodiments, -Cy^{L1}- is

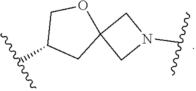 [SMILES: *[C@@H]1COC2(C1)CN(*)C2]

In some embodiments, -Cy^{L1}- is

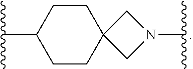 [SMILES: *C1CCC2(CC1)CN(*)C2]

In some embodiments, -Cy^{L1}- is

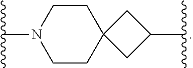 [SMILES: *C1CC2(CCN(*)CC2)C1]

In some embodiments, -Cy^{L1}- is

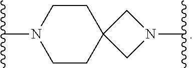 [SMILES: *N1CCC2(CC1)CN(*)C2]

In some embodiments, -Cy^{L1}- is

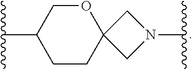 [SMILES: *C1CCC2(CN(*)C2)OC1]

In some embodiments, -Cy^{L1}- is

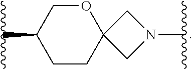 [SMILES: *[C@@H]1CCC2(CN(*)C2)OC1]

In some embodiments, -Cy^{L1}- is

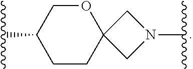 [SMILES: *[C@H]1CCC2(CN(*)C2)OC1]

In some embodiments, -Cy^{L1}- is

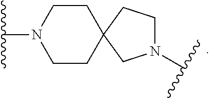 [SMILES: *N1CCC2(CC1)CCN(*)C2]

In some embodiments, -Cy^{L1}- is

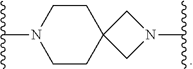 [SMILES: *N1CCC2(CC1)CN(*)C2]

In some embodiments, -Cy^{L1}- is

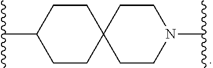 [SMILES: *C1CCC2(CC1)CCN(*)CC2]

In some embodiments, -Cy^{L1}- is

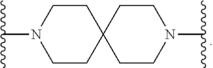 [SMILES: *N1CCC2(CC1)CCN(*)CC2]

In some embodiments, -Cy^{L1}- is

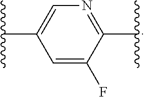 [SMILES: *c1cnc(*)c(F)c1]

In some embodiments, -Cy^{L1}- is an optionally substituted 8-10 membered bicyclic arylenyl. In some embodiments, -Cy^{L1}- is an optionally substituted 3-7 membered saturated or partially unsaturated carbocyclylenyl. In some embodiments, -Cy^{L2}- is an optionally substituted 6-1l membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is an optionally substituted a 4-7 membered saturated or partially unsaturated heterocyclylenyl having 1-2 heteroatoms independently selected from nitrogen, oxygen, and sulfur. In some embodiments, -Cy^{L2}- is an optionally substituted 6-11 membered saturated or partially unsaturated spiro heterocyclylenyl having 1-3 heteroatoms independently selected from nitrogen, oxygen, and sulfur. In some embodiments, -Cy^{L2}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L2}- is an optionally substituted 5-6 membered heteroarylenyl having 1-4 heteroatoms independently selected from nitrogen, oxygen. In some embodiments, -Cy^{L2}- is an optionally substituted 8-10 membered bicyclic heteroarylenyl having 1-5 heteroatoms independently selected from nitrogen, oxygen, and sulfur.

In some embodiments, each -Cy^{L2}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an 8-10 membered bicyclic arylenyl. In some embodiments, -Cy^{L1}- is a 3-7 membered saturated or partially unsaturated carbocyclylenyl. In some embodiments, -Cy^{L2}- is a 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is a 4-7 membered saturated or partially unsaturated heterocyclylenyl having 1-2 heteroatoms independently selected from nitrogen, oxygen, and sulfur. In some embodiments, -Cy^{L2}- is a 6-11 membered saturated or partially unsaturated spiro heterocyclylenyl having 1-3 heteroatoms independently selected from nitrogen, oxygen, and sulfur. In some embodiments, -Cy^{L2}- is an 8-10 membered bicyclic saturated or partially unsaturated heterocyclylenyl having 1-2 heteroatoms independently selected from nitrogen, oxygen, and sulfur. In some embodiments, -Cy^{L2}- is a 5-6 membered heteroarylenyl having 1-4 heteroatoms independently selected from nitrogen, oxygen. In some embodiments, -Cy^{L2}- is an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L2}- is an optionally substituted phenylenyl. In some embodiments, -Cy^{L2}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L2}- is an optionally substituted 5-6 membered saturated or partially unsaturated heterocyclylenyl having 1-2 heteroatoms independently selected from nitrogen, oxygen, and sulfur. In some embodiments, -Cy^{L2}- is an optionally substituted 5 membered saturated or partially unsaturated heterocyclylenyl having 1-2 heteroatoms independently selected from nitrogen, oxygen, and sulfur. In some embodiments, -Cy^{L2}- is an optionally substituted pyrrolidinylenyl. In some embodiments, -Cy^{L2}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyrrolidinyl, optionally substituted with halogen (e.g., fluoro or chloro) or C₁₋₆ aliphatic. In some embodiments, -Cy^{L2}- is an optionally substituted 6 membered saturated or partially unsaturated heterocyclylenyl having 1-2 heteroatoms independently selected from nitrogen, oxygen, and sulfur. In some embodiments, -Cy^{L2}- is an optionally substituted piperadinylenyl. In some embodiments, -Cy^{L2}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is piperadinylenyl, optionally substituted with halogen (e.g., fluoro or chloro) or C₁₋₆ aliphatic. In some embodiments, -Cy^{L2}- is an optionally substituted piperazinylenyl. In some embodiments, -Cy^{L2}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C a aliphatic. In some embodiments, -Cy^{L2}- is piperazinylenyl, optionally substituted with halogen (e.g., fluoro or chloro) or C₁₋₆ aliphatic.

In some embodiments, -Cy^{L2}- is an optionally substituted 5 membered heteroarylenyl having 1-4 heteroatoms independently selected from nitrogen, oxygen. In some embodiments, -Cy^{L2}- is an optionally substituted pyrrolylenyl, pyrazolylenyl, imidazolylenyl, or triazolylenyl. In some embodiments, -Cy^{L2}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L2}- is an optionally substituted pyrazolylenyl. In some embodiments, -Cy^{L2}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyrazolylenyl, optionally substituted with halogen (e.g., fluoro or chloro) or C₁₋₆ aliphatic. In some embodiments, -Cy^{L2}- is an optionally substituted imidazolylenyl. In some embodiments, -Cy^{L2}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L2}- is imidazolylenyl, optionally substituted with halogen (e.g., fluoro or chloro) or C₁₋₆ aliphatic.

In some embodiments, -Cy^{L2}- is an optionally substituted 6 membered heteroarylenyl having 1-4 heteroatoms independently selected from nitrogen, oxygen. In some embodiments, -Cy^{L2}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₃ aliphatic. In some embodiments, -Cy^{L2}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L2}- is an optionally substituted pyridinylenyl. In some embodiments, -Cy^{L2}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridinylenyl, optionally substituted with halogen (e.g., fluoro or chloro) or C₁₋₆ aliphatic. In some embodiments, -Cy^{L2}- is an optionally substituted pyridazinylenyl. In some embodiments, -Cy^{L2}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridazinylenyl, optionally substituted with halogen (e.g., fluoro or chloro) or C₁₋₆ aliphatic. In some embodiments, -Cy^{L2}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L2}- is an optionally substituted pyridinonylenyl. In some embodiments, -Cy^{L2}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, optionally substituted with halogen (e.g., fluoro or chloro) or C₁₋₆ aliphatic.

In some embodiments, -Cy^{L2}- is an optionally substituted cyclopropyl. In some embodiments, -Cy^{L1}- is an optionally substituted 5 membered saturated or partially unsaturated carbocyclylenyl. In some embodiments, -Cy^{L2}- is an optionally substituted cyclopentanylenyl or cyclopentenylenyl. In some embodiments, -Cy^{L2}- is an optionally substituted 6 membered saturated or partially unsaturated carbocyclylenyl. In some embodiments, -Cy^{L2}- is an optionally substituted cyclohexanylenyl or cyclohexenylenyl. In some embodiments, -Cy^{L2}- is an optionally substituted naphthalenylenyl.

In some embodiments, -Cy^{L2}- is an optionally substituted 9 membered bicyclic heteroarylenyl having 1-5 heteroatoms independently selected from nitrogen, oxygen, and sulfur. In some embodiments, -Cy^{L2}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L2}- is an optionally substituted indolylenyl. In some embodiments, -Cy^{L2}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L2}- is an optionally substituted 8-11 membered saturated or partially unsaturated spiro heterocyclylenyl having 1-3 heteroatoms independently selected from nitrogen, oxygen, and sulfur. In some embodiments, -Cy^{L2}- is an optionally substituted 9-11 membered saturated or partially unsaturated spiro heterocyclylenyl having 1-3 heteroatoms independently selected from nitrogen, oxygen, and sulfur. In some embodiments, -Cy^{L2}- is an optionally substituted 9 membered saturated or partially unsaturated spiro heterocyclylenyl having 1-3 heteroatoms independently selected from nitrogen, oxygen, and sulfur. In some embodiments, -Cy^{L2}- is an optionally substituted 9 membered saturated or partially unsaturated spiro heterocyclylenyl having 1-3 nitrogen heteroatoms. In some embodiments, -Cy^{L2}- is an optionally substituted 4,6-spiro heterocyclylenyl having 1-3 heteroatoms independently selected from nitrogen, oxygen, and sulfur. In some embodiments, -Cy^{L1}- is an optionally substituted

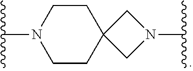 [SMILES: *N1CCC2(CC1)CN(*)C2]

In some embodiments, -Cy^{L2}- is an optionally substituted 10 membered saturated or partially unsaturated spiro heterocyclylenyl having 1-3 heteroatoms independently selected from nitrogen, oxygen, and sulfur. In some embodiments, -Cy^{L2}- is an optionally substituted 10 membered saturated or partially unsaturated spiro heterocyclylenyl having 1-3 nitrogen heteroatoms. In some embodiments, -Cy^{L2}- is an optionally substituted 5,6-spiro heterocyclylenyl having 1-3 heteroatoms independently selected from nitrogen, oxygen, and sulfur.

In some embodiments, -Cy^{L1}- is an optionally substituted 11 membered saturated or partially unsaturated spiro heterocyclylenyl having 1-3 heteroatoms independently selected from nitrogen, oxygen, and sulfur. In some embodiments, -Cy^{L1}- is an optionally substituted 11 membered saturated or partially unsaturated spiro heterocyclylenyl having 1-3 nitrogen heteroatoms. In some embodiments, -Cy^{L2}- is an optionally substituted 6,6-spiro heterocyclylenyl having 1-3 heteroatoms independently selected from nitrogen, oxygen, and sulfur.

In some embodiments, -Cy^{L2}- is optionally substituted

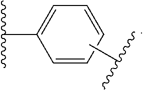

In some embodiments, -Cy^{L2}- is

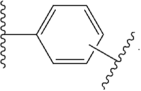

In some embodiments, -Cy^{L2}- is

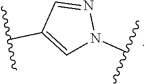 [SMILES: *c1cnn(*)c1]

In some embodiments, -Cy^{L2}- is

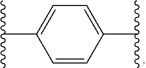 [SMILES: *c1ccc(*)cc1]

In some embodiments, -Cy^{L2}- is

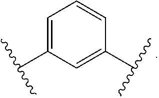 [SMILES: *c1cccc(*)c1]

In some embodiments, -Cy^{L2}- is

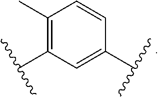 [SMILES: *c1ccc(C)c(*)c1]

In some embodiments, -Cy^{L2}- is

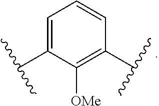 [SMILES: *c1cccc(*)c1OC]

In some embodiments, -Cy^{L2}- is

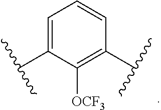 [SMILES: *c1cccc(*)c1OC(F)(F)F]

In some embodiments, -Cy^{L2}- is

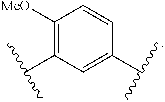 [SMILES: *c1ccc(OC)c(*)c1]

In some embodiments, -Cy^{L2}- is

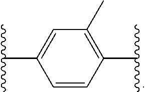 [SMILES: *c1ccc(*)c(C)c1]

In some embodiments, -Cy^{L2}- is

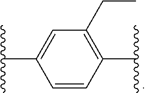 [SMILES: *c1ccc(*)c(CC)c1]

In some embodiments, -Cy^{L2}- is

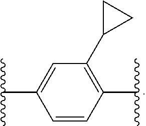 [SMILES: *c1ccc(*)c(C2CC2)c1]

In some embodiments, -Cy^{L2}- is

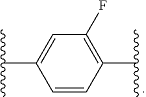 [SMILES: *c1ccc(*)c(F)c1]

In some embodiments, -Cy^{L2}- is

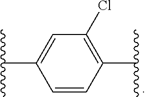 [SMILES: *c1ccc(*)c(Cl)c1]

In some embodiments, -Cy^{L2}- is

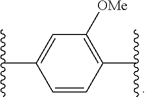 [SMILES: *c1ccc(*)c(OC)c1]

In some embodiments, -Cy^{L2}- is

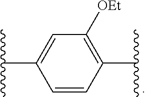 [SMILES: *c1ccc(*)c(OCC)c1]

In some embodiments, -Cy^{L2}- is

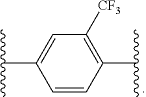 [SMILES: *c1ccc(*)c(C(F)(F)F)c1]

In some embodiments, -Cy^{L2}- is

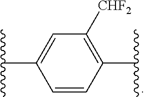 [SMILES: *c1ccc(*)c(C(F)F)c1]

In some embodiments, -Cy^{L2}- is

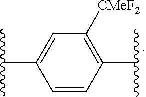

In some embodiments, -Cy^{L2}- is

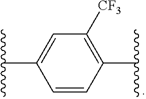 [SMILES: *c1ccc(*)c(C(F)(F)F)c1]

In some embodiments, -Cy^{L2}- is

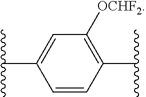 [SMILES: *c1ccc(*)c(OC(F)F)c1]

In some embodiments, -Cy^{L2}- is

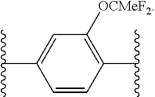

In some embodiments, -Cy^{L2}- is

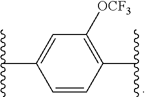 [SMILES: *c1ccc(*)c(OC(F)(F)F)c1]

In some embodiments, -Cy^{L2}- is

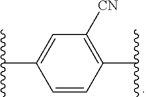 [SMILES: *c1ccc(*)c(C#N)c1]

In some embodiments, -Cy^{L2}- is

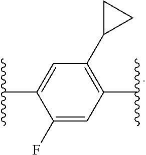 [SMILES: *c1cc(C2CC2)c(*)cc1F]

In some embodiments, -Cy^{L2}- is

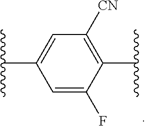 [SMILES: *c1cc(F)c(*)c(C#N)c1]

In some embodiments, -Cy^{L2}- is

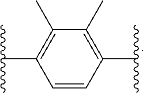 [SMILES: *c1ccc(*)c(C)c1C]

In some embodiments, -Cy^{L2}- is

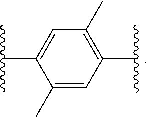 [SMILES: *c1cc(C)c(*)cc1C]

In some embodiments, -Cy^{L2}- is

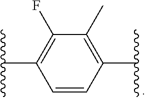 [SMILES: *c1ccc(*)c(F)c1C]

In some embodiments, -Cy^{L2}- is

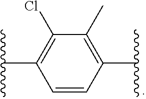 [SMILES: *c1ccc(*)c(Cl)c1C]

In some embodiments, -Cy^{L2}- is

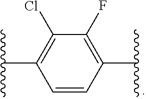 [SMILES: *c1ccc(*)c(Cl)c1F]

In some embodiments, -Cy^{L2}- is

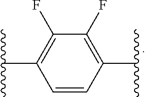 [SMILES: *c1ccc(*)c(F)c1F]

In some embodiments, -Cy^{L2}- is

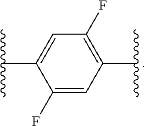 [SMILES: *c1cc(F)c(*)cc1F]

In some embodiments, -Cy^{L2}- is

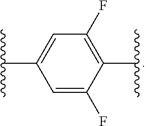 [SMILES: *c1cc(F)c(*)c(F)c1]

In some embodiments, -Cy^{L2}- is

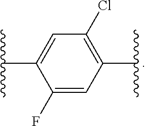 [SMILES: *c1cc(Cl)c(*)cc1F]

In some embodiments, -Cy^{L2}- is

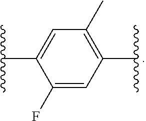 [SMILES: *c1cc(F)c(*)cc1C]

In some embodiments, -Cy^{L2}- is

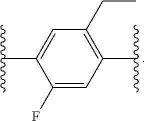 [SMILES: *c1cc(CC)c(*)cc1F]

In some embodiments, -Cy^{L2}- is

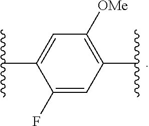 [SMILES: *c1cc(OC)c(*)cc1F]

In some embodiments, -Cy^{L2}- is

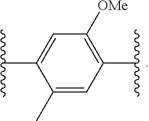 [SMILES: *c1cc(OC)c(*)cc1C]

In some embodiments, -Cy^{L2}- is

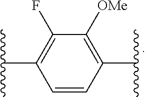 [SMILES: *c1ccc(*)c(OC)c1F]

In some embodiments, -Cy^{L2}- is

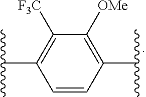 [SMILES: *c1ccc(*)c(C(F)(F)F)c1OC]

In some embodiments, -Cy^{L2}- is

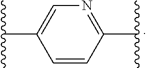 [SMILES: *c1ccc(*)nc1 ; "."]

In some embodiments, -Cy^{L2}- is

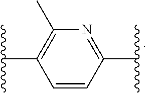 [SMILES: *c1ccc(*)c(C)n1 ; "."]

In some embodiments, -Cy^{L2}- is

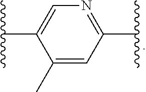 [SMILES: *c1cc(C)c(*)cn1]

In some embodiments, -Cy^{L2}- is

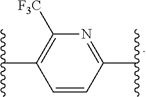 [SMILES: *c1ccc(*)c(C(F)(F)F)n1]

In some embodiments, -Cy^{L2}- is

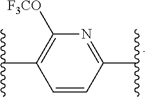 [SMILES: *c1ccc(*)c(OC(F)(F)F)n1]

In some embodiments, -Cy^{L2}- is

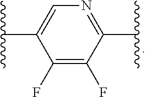 [SMILES: *c1cnc(*)c(F)c1F]

In some embodiments, -Cy^{L2}- is

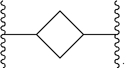 [SMILES: *C1CC(*)C1]

In some embodiments, -Cy^{L2}- is

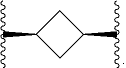 [SMILES: *[C@H]1C[C@@H](*)C1]

In some embodiments, -Cy^{L2}- is

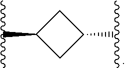 [SMILES: *[C@H]1C[C@H](*)C1]

In some embodiments, -Cy^{L2}- is

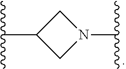 [SMILES: *C1CN(*)C1]

In some embodiments, -Cy^{L2}- is

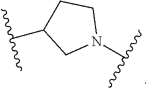 [SMILES: *C1CCN(*)C1]

In some embodiments, -Cy^{L2}- is

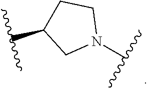 [SMILES: *[C@H]1CCN(*)C1]

In some embodiments, -Cy^{L2}- is

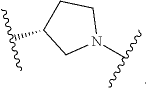 [SMILES: *[C@@H]1CCN(*)C1]

In some embodiments, -Cy^{L2}- is

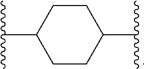 [SMILES: *C1CCC(*)CC1]

In some embodiments, -Cy^{L2}- is

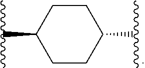 [SMILES: *[C@H]1CC[C@H](*)CC1]

In some embodiments, -Cy^{L2}- is

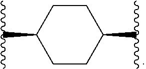 [SMILES: *[C@H]1CC[C@@H](*)CC1]

In some embodiments, -Cy^{L2}- is

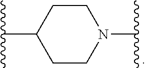 [SMILES: *C1CCN(*)CC1]

In some embodiments, -Cy^{L2}- is

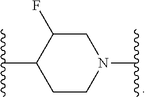 [SMILES: *C1CCN(*)CC1F]

In some embodiments, -Cy^{L2}- is

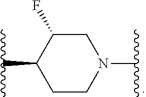 [SMILES: *[C@@H]1CCN(*)C[C@H]1F]

In some embodiments, -Cy^{L2}- is

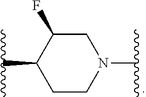 [SMILES: *[C@@H]1CCN(*)C[C@@H]1F]

In some embodiments, -Cy^{L2}- is

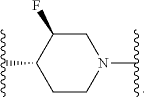 [SMILES: *[C@H]1CCN(*)C[C@@H]1F]

In some embodiments, -Cy^{L2}- is

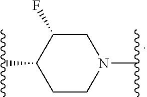 [SMILES: *[C@H]1CCN(*)C[C@H]1F ; "."]

In some embodiments, -Cy^{L2}- is

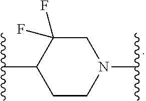 [SMILES: *C1CCN(*)CC1(F)F]

In some embodiments, -Cy^{L2}- is

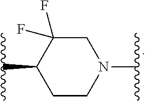 [SMILES: *[C@@H]1CCN(*)CC1(F)F]

In some embodiments, -Cy^{L2}- is

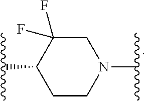 [SMILES: *[C@H]1CCN(*)CC1(F)F]

In some embodiments, -Cy^{L2}- is

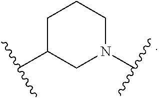 [SMILES: *C1CCCN(*)C1 ; "."]

In some embodiments, -Cy^{L2}- is

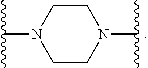 [SMILES: *N1CCN(*)CC1]

In some embodiments, -Cy^{L2}- is

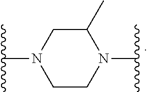 [SMILES: *N1CCN(*)C(C)C1]

In some embodiments, -Cy^{L2}- is

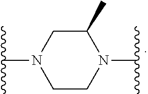 [SMILES: *N1CCN(*)[C@H](C)C1]

In some embodiments, -Cy^{L2}- is

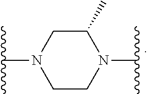 [SMILES: *N1CCN(*)[C@@H](C)C1]

In some embodiments, -Cy^{L2}- is

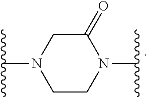 [SMILES: *N1CCN(*)C(=O)C1]

In some embodiments, -Cy^{L2}- is

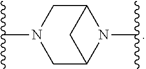 [SMILES: *N1CC2CC(C1)N2*]

In some embodiments, -Cy^{L2}- is

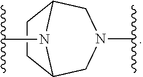 [SMILES: *N1CC2CCC(C1)N2*]

In some embodiments, -Cy^{L2}- is

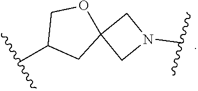 [SMILES: *C1COC2(C1)CN(*)C2]

In some embodiments, -Cy^{L2}- is

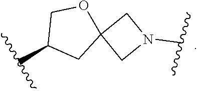 [SMILES: *[C@H]1COC2(C1)CN(*)C2]

In some embodiments, -Cy^{L2}- is

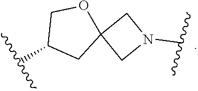 [SMILES: *[C@@H]1COC2(C1)CN(*)C2]

In some embodiments, -Cy^{L2}- is

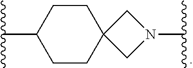 [SMILES: *C1CCC2(CC1)CN(*)C2]

In some embodiments, -Cy^{L2}- is

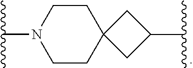 [SMILES: *C1CC2(CCN(*)CC2)C1]

In some embodiments, -Cy^{L2}- is

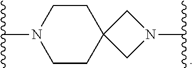 [SMILES: *N1CCC2(CC1)CN(*)C2]

In some embodiments, -Cy^{L2}- is

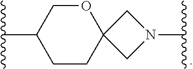 [SMILES: *C1CCC2(CN(*)C2)OC1]

In some embodiments, -Cy^{L2}- is

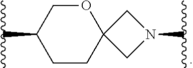

In some embodiments, -Cy^{L2}- is

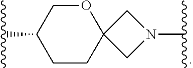 [SMILES: *[C@H]1CCC2(CN(*)C2)OC1]

In some embodiments, -Cy^{L2}- is

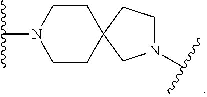 [SMILES: *N1CCC2(CC1)CCN(*)C2]

In some embodiments, -Cy^{L2}- is

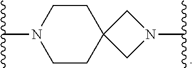 [SMILES: *N1CCC2(CC1)CN(*)C2]

In some embodiments, -Cy^{L2}- is

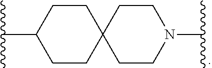 [SMILES: *C1CCC2(CC1)CCN(*)CC2]

In some embodiments, -Cy^{L2}- is

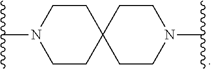 [SMILES: *N1CCC2(CC1)CCN(*)CC2]

In some embodiments, -Cy^{L2}- is

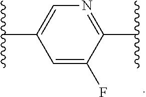 [SMILES: *c1cnc(*)c(F)c1]

In some embodiments -Cy^{L3}- is an optionally substituted 8-10 membered bicyclic arylenyl. In some embodiments, -Cy^{L3}- is an optionally substituted 3-7 membered saturated or partially unsaturated carbocyclylenyl. In some embodiments, -Cy^{L3}- is an optionally substituted 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3}- is an optionally substituted a 4-7 membered saturated or partially unsaturated heterocyclylenyl having 1-2 heteroatoms independently selected from nitrogen, oxygen, and sulfur. In some embodiments, -Cy^{L3}- is an optionally substituted 6-11 membered saturated or partially unsaturated spiro heterocyclylenyl having 1-3 heteroatoms independently selected from nitrogen, oxygen, and sulfur. In some embodiments, -Cy^{L3}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L1}- is an optionally substituted 5-6 membered heteroarylenyl having 1-4 heteroatoms independently selected from nitrogen, oxygen. In some embodiments, -Cy^{L1}- is an optionally substituted 8-10 membered bicyclic heteroarylenyl having 1-5 heteroatoms independently selected from nitrogen, oxygen, and sulfur.

In some embodiments, each -Cy^{L3}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L3}- is an 8-10 membered bicyclic arylenyl. In some embodiments, -Cy^{L3}- is a 3-7 membered saturated or partially unsaturated carbocyclylenyl. In some embodiments, -Cy^{L3}- is a 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3}- is a 4-7 membered saturated or partially unsaturated heterocyclylenyl having 1-2 heteroatoms independently selected from nitrogen, oxygen, and sulfur. In some embodiments, -Cy^{L3}- is a 6-11 membered saturated or partially unsaturated spiro heterocyclylenyl having 1-3 heteroatoms independently selected from nitrogen, oxygen, and sulfur. In some embodiments, -Cy^{L3}- is an 8-10 membered bicyclic saturated or partially unsaturated heterocyclylenyl having 1-2 heteroatoms independently selected from nitrogen, oxygen, and sulfur. In some embodiments, -Cy^{L3}- is a 5-6 membered heteroarylenyl having 1-4 heteroatoms independently selected from nitrogen, oxygen. In some embodiments, -Cy^{L3}- is an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L3}- is an optionally substituted phenylenyl. In some embodiments, -Cy^{L1}- is phenylenyl, optionally substituted with halogen (e.g., fluoro or chloro), —CN, C₁e aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L3}- is an optionally substituted 5-6 membered saturated or partially unsaturated heterocyclylenyl having 1-2 heteroatoms independently selected from nitrogen, oxygen, and sulfur. In some embodiments, -Cy^{L3}- is an optionally substituted 5 membered saturated or partially unsaturated heterocyclylenyl having 1-2 heteroatoms independently selected from nitrogen, oxygen, and sulfur. In some embodiments, -Cy^{L3}- is an optionally substituted pyrrolidinylenyl. In some embodiments, -Cy^{L3}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o} wherein R^{o} is hydrogen or C₁₋₆ aliphatic. In some embodiments, -Cy^{L3}- is pyrrolidinylenyl, optionally substituted with halogen (e.g., fluoro or chloro) or C₁₋₆ aliphatic. In some embodiments, -Cy^{L3}- is an optionally substituted 6 membered saturated or partially unsaturated heterocyclylenyl having 1-2 heteroatoms independently selected from nitrogen, oxygen, and sulfur. In some embodiments, -Cy^{L3}- is an optionally substituted piperadinylenyl. In some embodiments, -Cy^{L3}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is piperadinylenyl, optionally substituted with halogen (e.g., fluoro or chloro) or C₁₋₆ aliphatic. In some embodiments, -Cy^{L3}- is an optionally substituted piperazinylenyl. In some embodiments, -Cy^{L3}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is piperazinylenyl, optionally substituted with halogen (e.g., fluoro or chloro) or C₁₋₆ aliphatic.

In some embodiments, -Cy^{L3}- is an optionally substituted 5 membered heteroarylenyl having 1-4 heteroatoms independently selected from nitrogen, oxygen. In some embodiments, -Cy^{L3}- is an optionally substituted pyrrolylenyl, pyrazolylenyl, imidazolylenyl, or triazolylenyl. In some embodiments, -Cy^{L3}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L3}- is an optionally substituted pyrazolylenyl. In some embodiments, -Cy^{L3}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyrazolylenyl, optionally substituted with halogen (e.g., fluoro or chloro) or C₁₋₆ aliphatic. In some embodiments, -Cy^{L3}- is an optionally substituted imidazolylenyl. In some embodiments, -Cy^{L3}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L3}- is imidazolylenyl, optionally substituted with halogen (e.g., fluoro or chloro) or C₁₋₆ aliphatic.

In some embodiments, -Cy^{L3}- is an optionally substituted 6 membered heteroarylenyl having 1-4 heteroatoms independently selected from nitrogen, oxygen. In some embodiments, -Cy^{L3}- is an optionally substituted pyridinylenyl, pyridazinylenvl, pyrimidinylenyl, pyrazinylenyl, or triazinylenyl. In some embodiments, -Cy^{L3}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L3}- is an optionally substituted pyridinylenyl. In some embodiments, -Cy^{L3}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinylenyl, optionally substituted with halogen (e.g., fluoro or chloro) or C₁₋₆ aliphatic. In some embodiments, -Cy^{L3}- is an optionally substituted pyridazinylenyl. In some embodiments, -Cy^{L3}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L2}- is pyridazinylenyl, optionally substituted with halogen (e.g., fluoro or chloro) or C₁₋₆ aliphatic. In some embodiments, -Cy^{L3}- is an optionally substituted pyridinonylenyl, pyridazinonylenyl, pyrimidinonylenyl, pyrazinonylenyl, or triazinonylenyl. In some embodiments, -Cy^{L3}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L3}- is an optionally substituted pyridinonylenyl. In some embodiments, -Cy^{L3}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L3}- is pyridinonylenyl, optionally substituted with halogen (e.g., fluoro or chloro) or C₁₋₆ aliphatic.

In some embodiments, -Cy^{L3}- is an optionally substituted cyclopropyl. In some embodiments, -Cy^{L3}- is an optionally substituted 5 membered saturated or partially unsaturated carbocyclylenyl. In some embodiments, -Cy^{L3}- is an optionally substituted cyclopentanylenyl or cyclopentenylenyl. In some embodiments, -Cy^{L3}- is an optionally substituted 6 membered saturated or partially unsaturated carbocyclylenyl. In some embodiments, -Cy^{L3}- is an optionally substituted cyclohexanylenyl or cyclohexenylenyl. In some embodiments, -Cy^{L3}- is an optionally substituted naphthalenylenyl.

In some embodiments, -Cy^{L3}- is an optionally substituted 9 membered bicyclic heteroarylenyl having 1-5 heteroatoms independently selected from nitrogen, oxygen, and sulfur. In some embodiments, -Cy^{L3}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L3}- is an optionally substituted indolylenyl. In some embodiments, -Cy^{L3}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L1}- is an optionally substituted 8-11 membered saturated or partially unsaturated spiro heterocyclylenyl having 1-3 heteroatoms independently selected from nitrogen, oxygen, and sulfur. In some embodiments, -Cy^{L3}- is an optionally substituted 9-11 membered saturated or partially unsaturated spiro heterocyclylenyl having 1-3 heteroatoms independently selected from nitrogen, oxygen, and sulfur. In some embodiments, -Cy^{L3}- is an optionally substituted 9 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 nitrogen heteroatoms. In some embodiments, -Cy^{L3}- is an optionally substituted 4,6-spiro heterocyclylenyl having 1-3 heteroatoms independently selected from nitrogen, oxygen, and sulfur. In some embodiments, -Cy^{L3}- is an optionally substituted

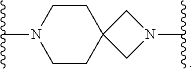 [SMILES: *N1CCC2(CC1)CN(*)C2]

In some embodiments, -Cy^{L3}- is an optionally substituted 10 membered saturated or partially unsaturated spiro heterocyclylenyl having 1-3 heteroatoms independently selected from nitrogen, oxygen, and sulfur. In some embodiments, -Cy^{L3}- is an optionally substituted 10 membered saturated or partially unsaturated spiro heterocyclylenyl having 1-3 nitrogen heteroatoms. In some embodiments, -Cy^{L3}- is an optionally substituted 5,6-spiro heterocyclylenyl having 1-3 heteroatoms independently selected from nitrogen, oxygen, and sulfur.

In some embodiments, -Cy^{L3}- is an optionally substituted 11 membered saturated or partially unsaturated spiro heterocyclylenyl having 1-3 heteroatoms independently selected from nitrogen, oxygen, and sulfur. In some embodiments, -Cy^{L3}- is an optionally substituted 11 membered saturated or partially unsaturated spiro heterocyclylenyl having 1-3 nitrogen heteroatoms. In some embodiments, -Cy^{L3}- is an optionally substituted 6,6-spiro heterocyclylenyl having 1-3 heteroatoms independently selected from nitrogen, oxygen, and sulfur.

In some embodiments, -Cy^{L3}- is optionally substituted

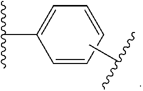

In some embodiments, -Cy^{L3}- is

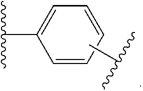

In some embodiments, -Cy^{L3}- is

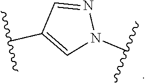 [SMILES: *c1cnn(*)c1]

In some embodiments, -Cy^{L3}- is

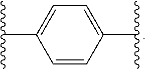 [SMILES: *c1ccc(*)cc1]

In some embodiments, -Cy^{L3}- is

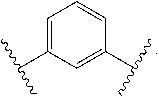 [SMILES: *c1cccc(*)c1]

In some embodiments, -Cy^{L3}- is

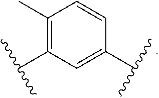 [SMILES: *c1ccc(C)c(*)c1]

In some embodiments, -Cy^{L3}- is

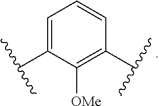 [SMILES: *c1cccc(*)c1OC]

In some embodiments, -Cy^{L3}- is

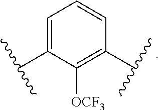 [SMILES: *c1cccc(*)c1OC(F)(F)F]

In some embodiments, -Cy^{L3}- is

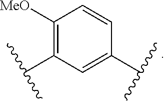 [SMILES: *c1ccc(OC)c(*)c1]

In some embodiments, -Cy^{L3}- is

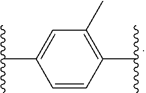 [SMILES: *c1ccc(*)c(C)c1]

In some embodiments, -Cy^{L3}- is

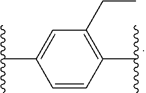 [SMILES: *c1ccc(*)c(CC)c1]

In some embodiments, -Cy^{L3}- is

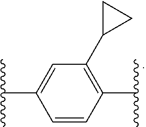 [SMILES: *c1ccc(*)c(C2CC2)c1]

In some embodiments, -Cy^{L3}- is

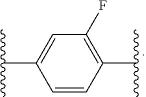 [SMILES: *c1ccc(*)c(F)c1]

In some embodiments, -Cy^{L3}- is

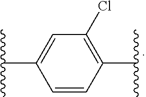 [SMILES: *c1ccc(*)c(Cl)c1]

In some embodiments, -Cy^{L3}- is

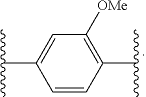 [SMILES: *c1ccc(*)c(OC)c1]

In some embodiments, -Cy^{L3}- is

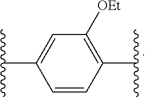 [SMILES: *c1ccc(*)c(OCC)c1]

In some embodiments, -Cy^{L3}- is

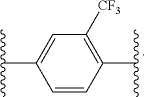 [SMILES: *c1ccc(*)c(C(F)(F)F)c1]

In some embodiments, -Cy^{L3}- is

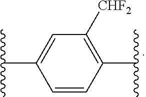 [SMILES: *c1ccc(*)c(C(F)F)c1]

In some embodiments, -Cy^{L3}- is

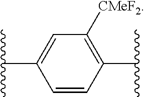

In some embodiments, -Cy^{L3}- is

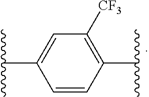 [SMILES: *c1ccc(*)c(C(F)(F)F)c1]

In some embodiments, -Cy^{L3}- is

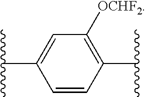 [SMILES: *c1ccc(*)c(OC(F)F)c1]

In some embodiments, -Cy^{L3}- is

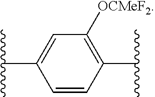

In some embodiments, -Cy^{L3}- is

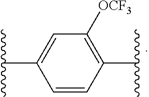 [SMILES: *c1ccc(*)c(OC(F)(F)F)c1]

In some embodiments, -Cy^{L3}- is

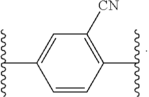 [SMILES: *c1ccc(*)c(C#N)c1]

In some embodiments, -Cy^{L3}- is

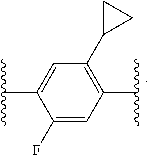 [SMILES: *c1cc(C2CC2)c(*)cc1F]

In some embodiments, -Cy^{L3}- is

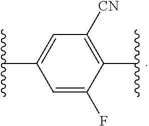 [SMILES: *c1cc(F)c(*)c(C#N)c1]

In some embodiments, -Cy^{L3}- is

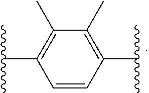 [SMILES: *c1ccc(*)c(C)c1C]

In some embodiments, -Cy^{L3}- is

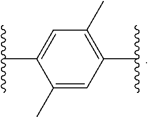 [SMILES: *c1cc(C)c(*)cc1C]

In some embodiments, -Cy^{L3}- is

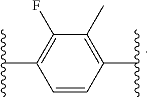 [SMILES: *c1ccc(*)c(F)c1C]

In some embodiments, -Cy^{L3}- is

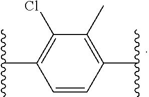 [SMILES: *c1ccc(*)c(Cl)c1C]

In some embodiments, -Cy^{L3}- is

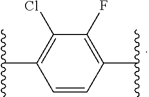 [SMILES: *c1ccc(*)c(Cl)c1F]

In some embodiments, -Cy^{L3}- is

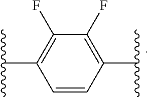 [SMILES: *c1ccc(*)c(F)c1F]

In some embodiments, -Cy^{L3}- is

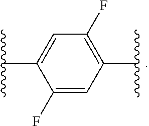 [SMILES: *c1cc(F)c(*)cc1F]

In some embodiments, -Cy^{L3}- is

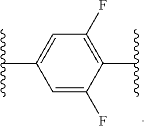 [SMILES: *c1cc(F)c(*)c(F)c1]

In some embodiments, -Cy^{L3}- is

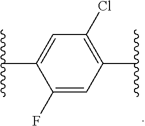 [SMILES: *c1cc(Cl)c(*)cc1F]

In some embodiments, -Cy^{L3}- is

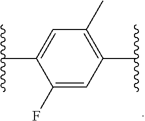 [SMILES: *c1cc(F)c(*)cc1C]

In some embodiments, -Cy^{L3}- is

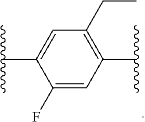 [SMILES: *c1cc(CC)c(*)cc1F]

In some embodiments, -Cy^{L3}- is

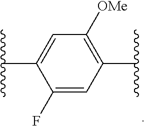 [SMILES: *c1cc(OC)c(*)cc1F]

In some embodiments, -Cy^{L3}- is

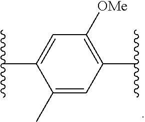 [SMILES: *c1cc(OC)c(*)cc1C]

In some embodiments, -Cy^{L3}- is

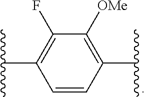 [SMILES: *c1ccc(*)c(OC)c1F]

In some embodiments, -Cy^{L3}- is

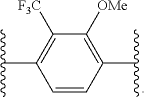 [SMILES: *c1ccc(*)c(C(F)(F)F)c1OC]

In some embodiments, -Cy^{L3}- is

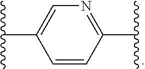 [SMILES: *c1ccc(*)nc1]

In some embodiments, -Cy^{L3}- is

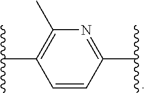 [SMILES: *c1ccc(*)c(C)n1]

In some embodiments, -Cy^{L3}- is

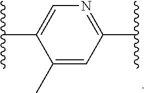 [SMILES: *c1cc(C)c(*)cn1]

In some embodiments, -Cy^{L3}- is

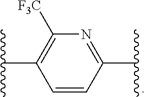 [SMILES: *c1ccc(*)c(C(F)(F)F)n1]

In some embodiments, -Cy^{L3}- is

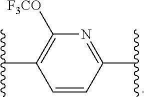 [SMILES: *c1ccc(*)c(OC(F)(F)F)n1]

In some embodiments, -Cy^{L3}- is

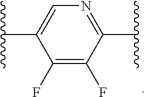 [SMILES: *c1cnc(*)c(F)c1F]

In some embodiments, -Cy^{L3}- is

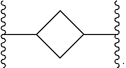 [SMILES: *C1CC(*)C1]

In some embodiments, -Cy^{L3}- is

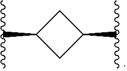 [SMILES: *[C@H]1C[C@@H](*)C1]

In some embodiments, -Cy^{L3}- is

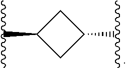 [SMILES: *[C@H]1C[C@H](*)C1]

In some embodiments, -Cy^{L3}- is

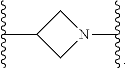 [SMILES: *C1CN(*)C1]

In some embodiments, -Cy^{L3}- is

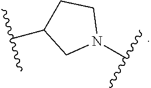 [SMILES: *C1CCN(*)C1]

In some embodiments, -Cy^{L3}- is

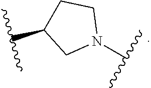 [SMILES: *[C@H]1CCN(*)C1]

In some embodiments, -Cy^{L3}- is

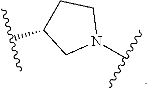 [SMILES: *[C@@H]1CCN(*)C1]

In some embodiments, -Cy^{L3}- is

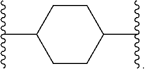 [SMILES: *C1CCC(*)CC1]

In some embodiments, -Cy^{L3}- is

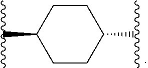 [SMILES: *[C@H]1CC[C@H](*)CC1]

In some embodiments, -Cy^{L3}- is

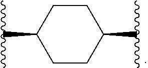 [SMILES: *[C@H]1CC[C@@H](*)CC1]

In some embodiments, -Cy^{L3}- is

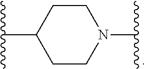 [SMILES: *C1CCN(*)CC1]

In some embodiments, -Cy^{L3}- is

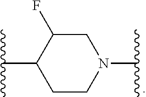 [SMILES: *C1CCN(*)CC1F]

In some embodiments, -Cy^{L3}- is

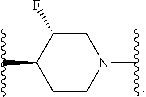 [SMILES: *[C@@H]1CCN(*)C[C@H]1F]

In some embodiments, -Cy^{L3}- is

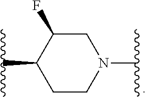 [SMILES: *[C@@H]1CCN(*)C[C@@H]1F]

In some embodiments, -Cy^{L3}- is

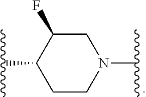 [SMILES: *[C@H]1CCN(*)C[C@@H]1F]

In some embodiments, -Cy^{L3}- is

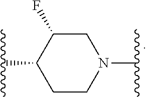 [SMILES: *[C@H]1CCN(*)C[C@H]1F ; "."]

In some embodiments, -Cy^{L3}- is

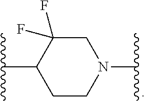 [SMILES: *C1CCN(*)CC1(F)F]

In some embodiments, -Cy^{L3}- is

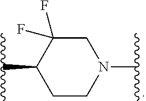 [SMILES: *[C@@H]1CCN(*)CC1(F)F]

In some embodiments, -Cy^{L3}- is

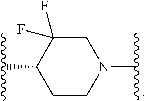 [SMILES: *[C@H]1CCN(*)CC1(F)F]

In some embodiments, -Cy^{L3}- is

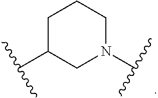 [SMILES: *C1CCCN(*)C1]

In some embodiments, -Cy^{L3}- is

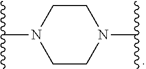 [SMILES: *N1CCN(*)CC1]

In some embodiments, -Cy^{L3}- is

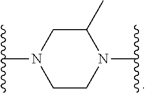 [SMILES: *N1CCN(*)C(C)C1]

In some embodiments, -Cy^{L3}- is

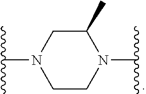 [SMILES: *N1CCN(*)[C@H](C)C1]

In some embodiments, -Cy^{L3}- is

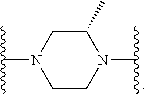 [SMILES: *N1CCN(*)[C@@H](C)C1]

In some embodiments, -Cy^{L3}- is

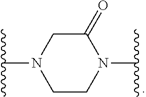 [SMILES: *N1CCN(*)C(=O)C1]

In some embodiments, -Cy^{L3}- is

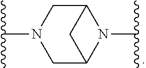 [SMILES: *N1CC2CC(C1)N2*]

In some embodiments, -Cy^{L3}- is

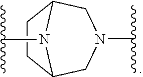 [SMILES: *N1CC2CCC(C1)N2*]

In some embodiments, -Cy^{L3}- is

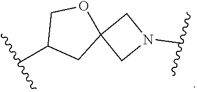 [SMILES: *C1COC2(C1)CN(*)C2]

In some embodiments, -Cy^{L3}- is

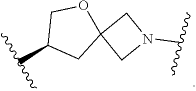 [SMILES: *[C@H]1COC2(C1)CN(*)C2]

In some embodiments, -Cy^{L3}- is

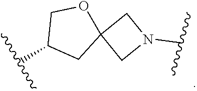 [SMILES: *[C@@H]1COC2(C1)CN(*)C2]

In some embodiments, -Cy^{L3}- is

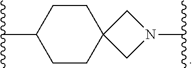 [SMILES: *C1CCC2(CC1)CN(*)C2]

In some embodiments, -Cy^{L3}- is

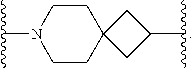 [SMILES: *C1CC2(CCN(*)CC2)C1]

In some embodiments, -Cy^{L3}- is

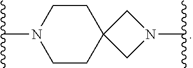 [SMILES: *N1CCC2(CC1)CN(*)C2]

In some embodiments, -Cy^{L3}- is

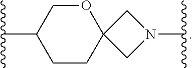 [SMILES: *C1CCC2(CN(*)C2)OC1]

In some embodiments, -Cy^{L3}- is

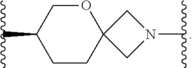 [SMILES: *[C@@H]1CCC2(CN(*)C2)OC1]

In some embodiments, -Cy^{L3}- is

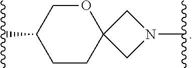 [SMILES: *[C@H]1CCC2(CN(*)C2)OC1]

In some embodiments, -Cy^{L3}- is

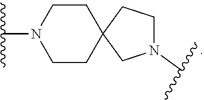 [SMILES: *N1CCC2(CC1)CCN(*)C2]

In some embodiments, -Cy^{L3}- is

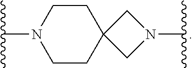 [SMILES: *N1CCC2(CC1)CN(*)C2]

In some embodiments, -Cy^{L3}- is

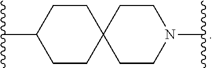 [SMILES: *C1CCC2(CC1)CCN(*)CC2]

In some embodiments, -Cy^{L3}- is

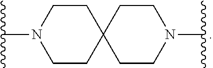 [SMILES: *N1CCC2(CC1)CCN(*)CC2]

In some embodiments, -Cy^{L3}- is

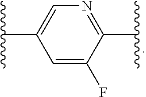 [SMILES: *c1cnc(*)c(F)c1]

In some embodiments, -Cy^{L1}- is an optionally substituted 8-10 membered bicyclic arylenyl. In some embodiments, -Cy^{L4}- is an optionally substituted 3-7 membered saturated or partially unsaturated carbocyclylenyl. In some embodiments, -Cy^{L4}- is an optionally substituted 6-11 membered saturated or partially unsaturated spiro carbocyclylenyl. In some embodiments, -Cy^{L4}- is an 8-10 membered bicyclic saturated or partially unsaturated carbocyclylenyl. In some embodiments, -Cy^{L4}- is an optionally substituted a 4-7 membered saturated or partially unsaturated heterocyclylenyl having 1-2 heteroatoms independently selected from nitrogen, oxygen, and sulfur. In some embodiments, -Cy^{L4}- is an optionally substituted 6-11 membered saturated or partially unsaturated spiro heterocyclylenyl having 1-3 heteroatoms independently selected from nitrogen, oxygen, and sulfur. In some embodiments, -Cy^{L4}- is an optionally substituted 8-10 membered bicyclic saturated or partially unsaturated heterocyclylenyl having 1-2 heteroatoms independently selected from nitrogen, oxygen, and sulfur. In some embodiments, —C^{L4}— is an optionally substituted 5-6 membered heteroarylenyl having 1-4 heteroatoms independently selected from nitrogen, oxygen. In some embodiments, -Cy^{L4}- is an optionally substituted 8-10 membered bicyclic heteroarylenyl having 1-5 heteroatoms independently selected from nitrogen, oxygen, and sulfur.

In some embodiments, each -Cy^{L4}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an 8-10 membered bicyclic arylenyl. In some embodiments, -Cy^{L4}- is a 3-7 membered saturated or partially unsaturated carbocyclylenyl. In some embodiments, -Cy^{L4}- is a 6-11 membered saturated or partially unsaturated spiro carbocyclylenyl. In some embodiments, -Cy^{L4}- is an 8-10 membered bicyclic saturated or partially unsaturated carbocyclylenyl. In some embodiments, -Cy^{L4}- is a 4-7 membered saturated or partially unsaturated heterocyclylenyl having 1-2 heteroatoms independently selected from nitrogen, oxygen, and sulfur. In some embodiments, -Cy^{L4}- is a 6-11 membered saturated or partially unsaturated spiro heterocyclylenyl having 1-3 heteroatoms independently selected from nitrogen, oxygen, and sulfur. In some embodiments, -Cy^{L4}- is an 8-10 membered bicyclic saturated or partially unsaturated heterocyclylenyl having 1-2 heteroatoms independently selected from nitrogen, oxygen, and sulfur. In some embodiments, -Cy^{L4}- is a 5-6 membered heteroarylenyl having 1-4 heteroatoms independently selected from nitrogen, oxygen. In some embodiments, -Cy^{L4}- is an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L4}- is an optionally substituted phenylenyl. In some embodiments, -Cy^{L4}- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L1}- is an optionally substituted 5-6 membered saturated or partially unsaturated heterocyclylenyl having 1-2 heteroatoms independently selected from nitrogen, oxygen, and sulfur. In some embodiments, -Cy^{L4}- is an optionally substituted 5 membered saturated or partially unsaturated heterocyclylenyl having 1-2 heteroatoms independently selected from nitrogen, oxygen, and sulfur. In some embodiments, -Cy^{L4}- is an optionally substituted pyrrolidinylenyl. In some embodiments, -Cy^{L1}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyrrolidinylenyl, optionally substituted with halogen (e.g., fluoro or chloro) or C₆ aliphatic. In some embodiments, -Cy^{L1}- is an optionally substituted 6 membered saturated or partially unsaturated heterocyclylenyl having 1-2 heteroatoms independently selected from nitrogen, oxygen, and sulfur. In some embodiments, -Cy^{L4}- is an optionally substituted piperadinylenyl. In some embodiments, -Cy^{L4}- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is piperadinylenyl, optionally substituted with halogen (e.g., fluoro or chloro) or C₁₋₆ aliphatic. In some embodiments, -Cy^{L4}- is an optionally substituted piperazinylenyl. In some embodiments, -Cy^{L4}- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is piperazinylenyl, optionally substituted with halogen (e.g., fluoro or chloro) or C₁₋₆ aliphatic.

In some embodiments, -Cy^{L4}- is an optionally substituted 5 membered heteroarylenyl having 1-4 heteroatoms independently selected from nitrogen, oxygen. In some embodiments, -Cy^{L4}- is an optionally substituted pyrrolylenyl, pyrazolylenyl, imidazolylenyl, or triazolylenyl. In some embodiments, -Cy^{L4}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₆ aliphatic. In some embodiments, -Cy^{L4}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L4}- is an optionally substituted pyrazolylenyl. In some embodiments, -Cy^{L4}- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyrazolylenyl, optionally substituted with halogen (e.g., fluoro or chloro) or C₁₋₆ aliphatic. In some embodiments, -Cy^{L4}- is an optionally substituted imidazolylenyl. In some embodiments, -Cy^{L4}- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy^{L4}- is imidazolylenyl, optionally substituted with halogen (e.g., fluoro or chloro) or C₁₋₆ aliphatic.

In some embodiments, -Cy^{L4}- is an optionally substituted 6 membered heteroarylenyl having 1-4 heteroatoms independently selected from nitrogen, oxygen. In some embodiments, -Cy^{L4}- is an optionally substituted pyridinylenyl, pyridazinylenyl, pyrimidinylenyl, pyrazinylenyl, or triazinylenyl. In some embodiments, -Cy^{L4}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L1}- is an optionally substituted pyridinylenyl. In some embodiments, -Cy^{L4}- is pyridinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁s aliphatic. In some embodiments, -Cy^{L4}- is pyridinylenyl, optionally substituted with halogen (e.g., fluoro or chloro) or C₁₋₆ aliphatic. In some embodiments, -Cy^{L4}- is an optionally substituted pyridazinylenyl. In some embodiments, -Cy^{L4}- is pyridazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L4}- is an optionally substituted pyridinonylenyl. In some embodiments, -Cy^{L4}- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy^{L1}- is pyridinonylenyl, optionally substituted with halogen (e.g., fluoro or chloro) or C₁₋₆ aliphatic.

In some embodiments, -Cy^{L1}- is an optionally substituted cyclopropyl. In some embodiments, -Cy^{L4}- is an optionally substituted 5 membered saturated or partially unsaturated carbocyclylenyl. In some embodiments, -Cy^{L4}- is an optionally substituted cyclopentanylenyl or cyclopentenylenyl. In some embodiments, -Cy^{L4}- is an optionally substituted 6 membered saturated or partially unsaturated carbocyclylenyl. In some embodiments, -Cy^{L4}- is an optionally substituted cyclohexanylenyl or cyclohexenylenyl. In some embodiments, -Cy^{L4}- is an optionally substituted naphthalenylenyl.

In some embodiments, -Cy^{L1}- is an optionally substituted 9 membered bicyclic heteroarylenyl having 1-5 heteroatoms independently selected from nitrogen, oxygen, and sulfur. In some embodiments, -Cy^{L4}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L4}- is an optionally substituted indolylenyl. In some embodiments, -Cy^{L4}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L4}- is an optionally substituted 8-11 membered saturated or partially unsaturated spiro heterocyclylenyl having 1-3 heteroatoms independently selected from nitrogen, oxygen, and sulfur. In some embodiments, -Cy^{L1}- is an optionally substituted 9-11 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 nitrogen heteroatoms. In some embodiments, -Cy^{L4}- is an optionally substituted 4,6-spiro heterocyclylenyl having 1-3 heteroatoms independently selected from nitrogen, oxygen, and sulfur. In some embodiments, -Cy^{L4}- is an optionally substituted

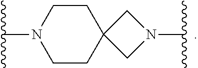 [SMILES: *N1CCC2(CC1)CN(*)C2]

In some embodiments, -Cy^{L4}- is an optionally substituted 10 membered saturated or partially unsaturated spiro heterocyclylenyl having 1-3 heteroatoms independently selected from nitrogen, oxygen, and sulfur. In some embodiments, -Cy^{L4}- is an optionally substituted 10 membered saturated or partially unsaturated spiro heterocyclylenyl having 1-3 nitrogen heteroatoms. In some embodiments, -Cy^{L4}- is an optionally substituted 5,6-spiro heterocyclylenyl having 1-3 heteroatoms independently selected from nitrogen, oxygen, and sulfur.

In some embodiments, -Cy^{L4}- is an optionally substituted 11 membered saturated or partially unsaturated spiro heterocyclylenyl having 1-3 heteroatoms independently selected from nitrogen, oxygen, and sulfur. In some embodiments, -Cy^{L4}- is an optionally substituted 11 membered saturated or partially unsaturated spiro heterocyclylenyl having 1-3 nitrogen heteroatoms. In some embodiments, -Cy^{L4}- is an optionally substituted 6,6-spiro heterocyclylenyl having 1-3 heteroatoms independently selected from nitrogen, oxygen, and sulfur. In some embodiments, -Cy^{L4}- is an optionally substituted

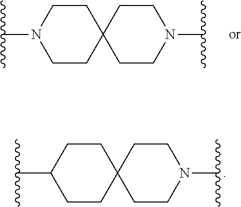 [SMILES: *N1CCC2(CC1)CCN(*)CC2 ; *C1CCC2(CC1)CCN(*)CC2]

In some embodiments, -Cy^{L4}- is optionally substituted

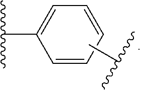

In some embodiments, -Cy^{L4}- is

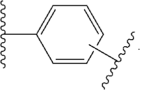

In some embodiments, -Cy^{L4}- is

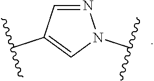 [SMILES: *c1cnn(*)c1]

In some embodiments, -Cy^{L4}- is

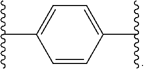 [SMILES: *c1ccc(*)cc1]

In some embodiments, -Cy^{L4}- is

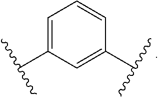 [SMILES: *c1cccc(*)c1]

In some embodiments, -Cy^{L4}- is

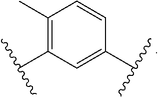 [SMILES: *c1ccc(C)c(*)c1]

In some embodiments, -Cy^{L4}- is

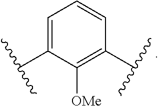 [SMILES: *c1cccc(*)c1OC]

In some embodiments, -Cy^{L4}- is

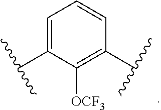 [SMILES: *c1cccc(*)c1OC(F)(F)F]

In some embodiments, -Cy^{L4}- is

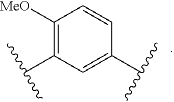 [SMILES: *c1ccc(OC)c(*)c1]

In some embodiments, -Cy^{L4}- is

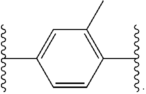 [SMILES: *c1ccc(*)c(C)c1]

In some embodiments, -Cy^{L4}- is

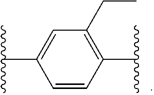 [SMILES: *c1ccc(*)c(CC)c1]

In some embodiments, -Cy^{L1}- is

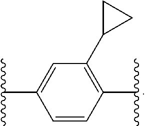 [SMILES: *c1ccc(*)c(C2CC2)c1]

In some embodiments, -Cy^{L4}- is

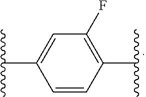 [SMILES: *c1ccc(*)c(F)c1]

In some embodiments, -Cy^{L4}- is

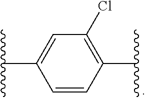 [SMILES: *c1ccc(*)c(Cl)c1]

In some embodiments, -Cy^{L4}- is

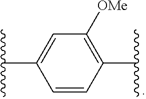 [SMILES: *c1ccc(*)c(OC)c1]

In some embodiments, -Cy^{L4}- is

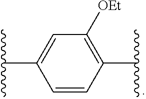 [SMILES: *c1ccc(*)c(OCC)c1]

In some embodiments, -Cy^{L4}- is

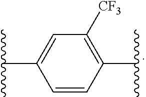 [SMILES: *c1ccc(*)c(C(F)(F)F)c1]

In some embodiments, -Cy^{L4}- is

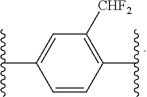 [SMILES: *c1ccc(*)c(C(F)F)c1]

In some embodiments, -Cy^{L4}- is

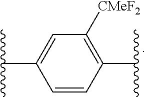

In some embodiments, -Cy^{L4}- is

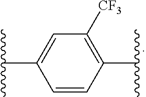 [SMILES: *c1ccc(*)c(C(F)(F)F)c1]

In some embodiments, -Cy^{L4}- is

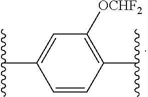 [SMILES: *c1ccc(*)c(OC(F)F)c1]

In some embodiments, -Cy^{L4}- is

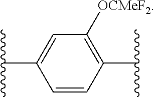

In some embodiments, -Cy^{L4}- is

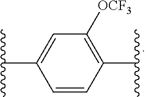 [SMILES: *c1ccc(*)c(OC(F)(F)F)c1]

In some embodiments, -Cy^{L4}- is

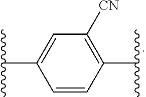 [SMILES: *c1ccc(*)c(C#N)c1]

In some embodiments, -Cy^{L4}- is

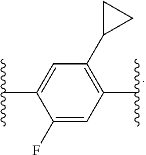 [SMILES: *c1cc(C2CC2)c(*)cc1F]

In some embodiments, -Cy^{L4}- is

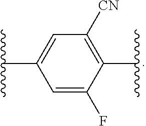 [SMILES: *c1cc(F)c(*)c(C#N)c1]

In some embodiments, -Cy^{L1}- is

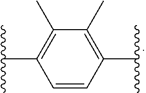 [SMILES: *c1ccc(*)c(C)c1C]

In some embodiments, -Cy^{L4}- is

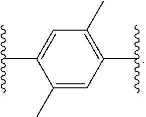 [SMILES: *c1cc(C)c(*)cc1C]

In some embodiments, -Cy^{L4}- is

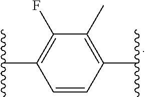 [SMILES: *c1ccc(*)c(F)c1C]

In some embodiments, -Cy^{L4}- is

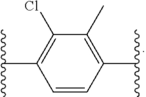 [SMILES: *c1ccc(*)c(Cl)c1C]

In some embodiments, -Cy^{L4}- is

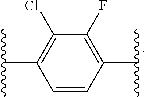 [SMILES: *c1ccc(*)c(Cl)c1F]

In some embodiments, -Cy^{L4}- is

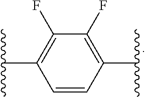 [SMILES: *c1ccc(*)c(F)c1F]

In some embodiments, -Cy^{L4}- is

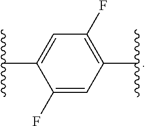 [SMILES: *c1cc(F)c(*)cc1F]

In some embodiments, -Cy^{L4}- is

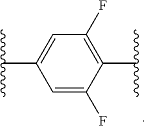 [SMILES: *c1cc(F)c(*)c(F)c1]

In some embodiments, -Cy^{L4}- is

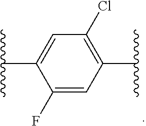 [SMILES: *c1cc(Cl)c(*)cc1F]

In some embodiments, -Cy^{L4}- is

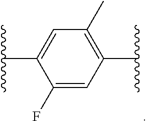 [SMILES: *c1cc(F)c(*)cc1C ; "."]

In some embodiments, -Cy^{L4}- is

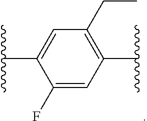 [SMILES: *c1cc(CC)c(*)cc1F ; "."]

In some embodiments, -Cy^{L4}- is

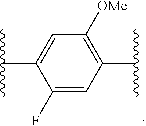 [SMILES: *c1cc(OC)c(*)cc1F]

In some embodiments, -Cy^{L4}- is

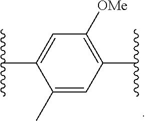 [SMILES: *c1cc(OC)c(*)cc1C]

In some embodiments, -Cy^{L4}- is

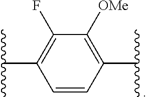 [SMILES: *c1ccc(*)c(OC)c1F ; "."]

In some embodiments, -Cy^{L4}- is

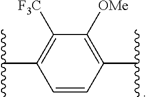 [SMILES: *c1ccc(*)c(C(F)(F)F)c1OC ; "."]

In some embodiments, -Cy^{L4}- is

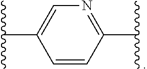 [SMILES: *c1ccc(*)nc1 ; "."]

In some embodiments, -Cy^{L4}- is

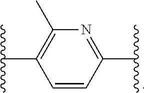 [SMILES: *c1ccc(*)c(C)n1]

In some embodiments, -Cy^{L4}- is

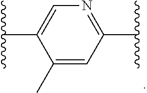 [SMILES: *c1cc(C)c(*)cn1]

In some embodiments, -Cy^{L4}- is

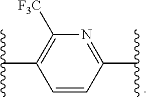 [SMILES: *c1ccc(*)c(C(F)(F)F)n1]

In some embodiments, -Cy^{L4}- is

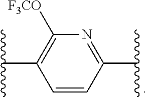 [SMILES: *c1ccc(*)c(OC(F)(F)F)n1]

In some embodiments, -Cy^{L4}- is

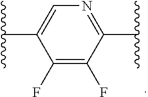 [SMILES: *c1cnc(*)c(F)c1F]

In some embodiments, -Cy^{L4}- is

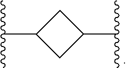 [SMILES: *C1CC(*)C1]

In some embodiments, -Cy^{L4}- is

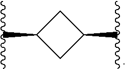 [SMILES: *[C@H]1C[C@@H](*)C1]

In some embodiments, -Cy^{L4}- is

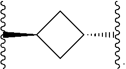 [SMILES: *[C@H]1C[C@H](*)C1]

In some embodiments, -Cy^{L4}- is

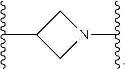 [SMILES: *C1CN(*)C1]

In some embodiments, -Cy^{L4}- is

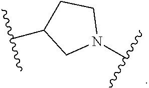 [SMILES: *C1CCN(*)C1]

In some embodiments, -Cy^{L4}- is

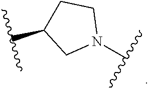 [SMILES: *[C@H]1CCN(*)C1]

In some embodiments, -Cy^{L4}- is

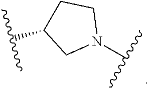 [SMILES: *[C@@H]1CCN(*)C1]

In some embodiments, -Cy^{L4}- is

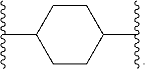 [SMILES: *C1CCC(*)CC1]

In some embodiments, -Cy^{L4}- is

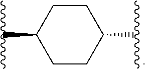 [SMILES: *[C@H]1CC[C@H](*)CC1]

In some embodiments, -Cy^{L4}- is

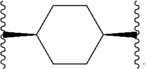 [SMILES: *[C@H]1CC[C@@H](*)CC1]

In some embodiments, -Cy^{L4}- is

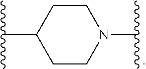 [SMILES: *C1CCN(*)CC1]

In some embodiments, -Cy^{L4}- is

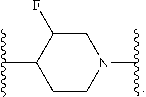 [SMILES: *C1CCN(*)CC1F]

In some embodiments, -Cy^{L4}- is

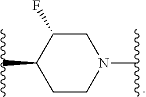 [SMILES: *[C@@H]1CCN(*)C[C@H]1F]

In some embodiments, -Cy^{L4}- is

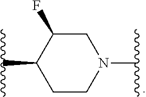 [SMILES: *[C@@H]1CCN(*)C[C@@H]1F]

In some embodiments, -Cy^{L4}- is

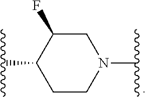 [SMILES: *[C@H]1CCN(*)C[C@@H]1F]

In some embodiments, -Cy^{L4}- is

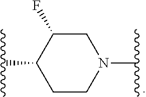 [SMILES: *[C@H]1CCN(*)C[C@H]1F]

In some embodiments, -Cy^{L4}- is

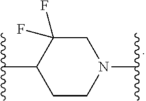 [SMILES: *C1CCN(*)CC1(F)F]

In some embodiments, -Cy^{L4}- is

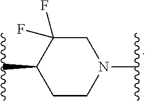 [SMILES: *[C@@H]1CCN(*)CC1(F)F]

In some embodiments, -Cy^{L4}- is

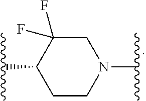 [SMILES: *[C@H]1CCN(*)CC1(F)F]

In some embodiments, -Cy^{L4}- is

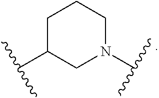 [SMILES: *C1CCCN(*)C1 ; "."]

In some embodiments, -Cy^{L4}- is

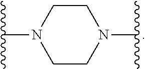 [SMILES: *N1CCN(*)CC1]

In some embodiments, -Cy^{L4}- is

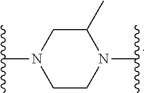 [SMILES: *N1CCN(*)C(C)C1]

In some embodiments, -Cy^{L4}- is

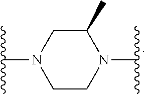 [SMILES: *N1CCN(*)[C@H](C)C1]

In some embodiments, -Cy^{L4}- is

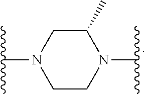 [SMILES: *N1CCN(*)[C@@H](C)C1]

In some embodiments, -Cy^{L4}- is

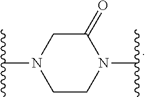 [SMILES: *N1CCN(*)C(=O)C1]

In some embodiments, -Cy^{L4}- is

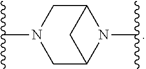 [SMILES: *N1CC2CC(C1)N2*]

In some embodiments, -Cy^{L4}- is

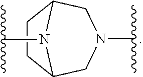 [SMILES: *N1CC2CCC(C1)N2*]

In some embodiments, -Cy^{L4}- is

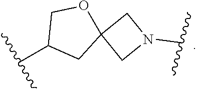 [SMILES: *C1COC2(C1)CN(*)C2]

In some embodiments, -Cy^{L4}- is

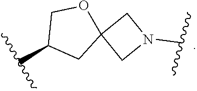 [SMILES: *[C@H]1COC2(C1)CN(*)C2]

In some embodiments, -Cy^{L4}- is

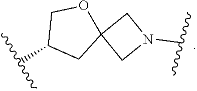 [SMILES: *[C@@H]1COC2(C1)CN(*)C2]

In some embodiments, -Cy^{L4}- is

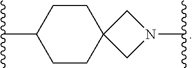 [SMILES: *C1CCC2(CC1)CN(*)C2]

In some embodiments, -Cy^{L4}- is

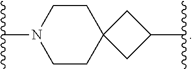 [SMILES: *C1CC2(CCN(*)CC2)C1]

In embodiments, -Cy^{L4}- is

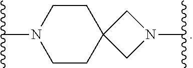 [SMILES: *N1CCC2(CC1)CN(*)C2]

In some embodiments, -Cy^{L4}- is

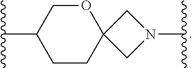 [SMILES: *C1CCC2(CN(*)C2)OC1]

In some embodiments, -Cy^{L4}- is

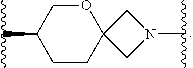 [SMILES: *[C@@H]1CCC2(CN(*)C2)OC1]

In some embodiments, -Cy^{L4}- is

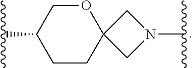 [SMILES: *[C@H]1CCC2(CN(*)C2)OC1]

In some embodiments, -Cy^{L4}- is

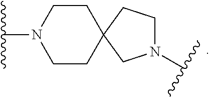 [SMILES: *N1CCC2(CC1)CCN(*)C2]

In some embodiments, -Cy^{L4}- is

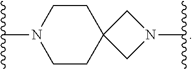 [SMILES: *N1CCC2(CC1)CN(*)C2]

In some embodiments, -Cy^{L4}- is

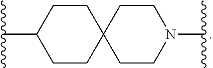 [SMILES: *C1CCC2(CC1)CCN(*)CC2]

In some embodiments, -Cy^{L4}- is

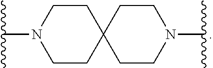 [SMILES: *N1CCC2(CC1)CCN(*)CC2]

In some embodiments, -Cy^{L4}- is

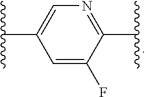 [SMILES: *c1cnc(*)c(F)c1]

In some embodiments, L is:

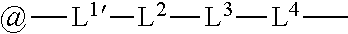

wherein
@ represents the point of attachment to Ring W;
L¹ is -Cy^{L1}-;
L² is -Cy^{L2}-;
L³ is -Cy^{L3}-; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

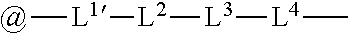

wherein
@ represents the point of attachment to Ring W;
L¹ is -Cy^{L3}-;
L² is -Cy^{L2}-;
L³ is -Cy^{L3}-; and
L⁴ is —C(O)—.

In some embodiments, L is:

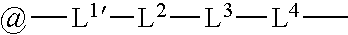

wherein
@ represents the point of attachment to Ring W;
L¹ is -Cy^{L1}-;
L² is -Cy^{L2}-;
L³ is -Cy^{L3}-;
L⁴ is a covalent bond or an optionally substituted bivalent, saturated or partially unsaturated, straight or branched C₁₋₆ hydrocarbon chain.

In some embodiments, L is:

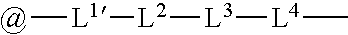

wherein
@ represents the point of attachment to Ring W;
L¹ is -Cy^{L1}-;
L² is -Cy^{L2}-;
L³ is -Cy^{L3}-;
L⁴ is an optionally substituted bivalent, saturated or partially unsaturated, straight or branched C₁₋₆ hydrocarbon chain.

In some embodiments, L is:

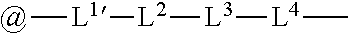

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-;
L³ is -Cy^{L3}-; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

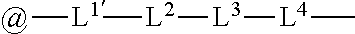

wherein
@ represents the point of attachment to Ring W;
L¹ is an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-;
L³ is -Cy^{L3}-; and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

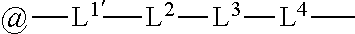

wherein
@ represents the point of attachment to Ring W;
L¹ is an optionally substituted bivalent, saturated or partially unsaturated, straight or branched C₁₋₆ hydrocarbon chain;
L² is -Cy^{L2}-;
L³ is -Cy^{L3}-; and
L⁴ is an optionally substituted bivalent, saturated or partially unsaturated, straight or branched C₁₋₆ hydrocarbon chain.

In some embodiments, L is

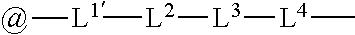

wherein
@ represents the point of attachment to Ring W;
L¹ is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy^{L3}-; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

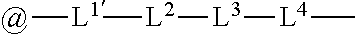

wherein
@ represents the point of attachment to Ring W;
L¹ is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy^{L3}-: and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

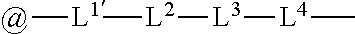

wherein
@ represents the point of attachment to Ring W;
L¹ is -Cy^{L1}-;
L² is an optionally substituted bivalent, saturated or partially unsaturated, straight or branched C₁₋₆ hydrocarbon chain;
L³ is -Cy^{L3}-; and
L⁴ is an optionally substituted bivalent, saturated or partially unsaturated, straight or branched C₁₋₆ hydrocarbon chain.

In some embodiments, L is:

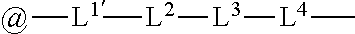

wherein
@ represents the point of attachment to Ring W;
L¹ is -Cy^{L1}-;
L² is -Cy^{L2}-;
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

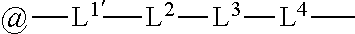

wherein
@ represents the point of attachment to Ring W;
L¹ is -Cy^{L1}-;
L² is -Cy^{L2}-;
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

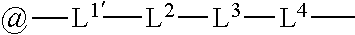

wherein
@ represents the point of attachment to Ring W;
L¹ is -Cy^{L1}-;
L² is -Cy^{L2}-;
L³ is an optionally substituted bivalent, saturated or partially unsaturated, straight or branched C₁₋₆ hydrocarbon chain; and
L⁴ is -Cy^{L4}-.

In some embodiments, L is:

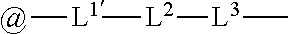

wherein
@ represents the point of attachment to Ring W;
L¹ is -Cy^{L1}-;
L² is -Cy^{L2}-; and
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

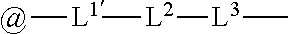

wherein
@ represents the point of attachment to Ring W;
L¹ is -Cy^{L1}-;
L² is -Cy^{L2}; and
L³ is a covalent bond or an optionally substituted bivalent, saturated or partially unsaturated, straight or branched C₁₋₆ hydrocarbon chain.

In some embodiments, L is:

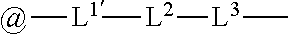

wherein
@ represents the point of attachment to Ring W;
L² is -Cy^{L1}-;
L² is -Cy^{L2}-; and
L³ is an optionally substituted bivalent, saturated or partially unsaturated, straight or branched Cu-hydrocarbon chain.

In some embodiments, L is:

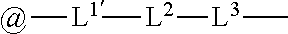

wherein
@ represents the point of attachment to Ring W;
L¹ is -Cy^{L1}-;
L² is -Cy^{L2}; and
L³ is -Cy^{L3}.

In some embodiments, L is:

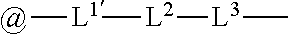

wherein
@ represents the point of attachment to Ring W;
L¹ is a covalent bond or an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}; and
L³ is a covalent bond or an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

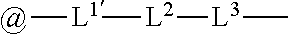

wherein
@ represents the point of attachment to Ring W;
L¹ is an optionally substituted bivalent, saturated or partially unsaturated, straight or branched C₁₋₆ hydrocarbon chain, wherein 1-2 methylene units of L¹ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}-; and
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

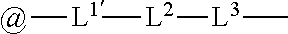

wherein
@ represents the point of attachment to Ring W;
L¹ is an optionally substituted bivalent, saturated or partially unsaturated, straight or branched C₁₋₆ hydrocarbon chain;
L² is -Cy^{L2}; and
L³ is an optionally substituted bivalent, saturated or partially unsaturated, straight or branched C₁₋₆ hydrocarbon chain.

In some embodiments, L is:

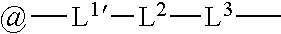

wherein
@ represents the point of attachment to Ring W;
L¹ is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy-L³;

In some embodiments, L is:

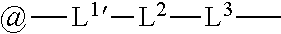

wherein
@ represents the point of attachment to Ring W;
L¹ is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy^{L3}-;

In some embodiments, L is:

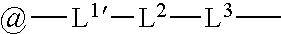

wherein
@ represents the point of attachment to Ring W;
L¹ is -Cy^{L1}-;
L² is an optionally substituted bivalent, saturated or partially unsaturated, straight or branched C₁₋₆ hydrocarbon chain; and
L³ is -Cy^{L3}-.

In some embodiments, L is:

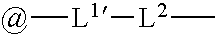

wherein
@ represents the point of attachment to Ring W;
L¹ is -Cy^{L1}-: and
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

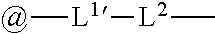

wherein
@ represents the point of attachment to Ring W;
L¹ is -Cy^{L1}-; and
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

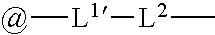

wherein
@ represents the point of attachment to Ring W;
L¹ is -Cy^{L1}-; and
L² is an optionally substituted bivalent, saturated or partially unsaturated, straight or branched C₁₋₆ hydrocarbon chain.

In some embodiments, L is:

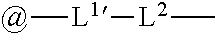

wherein
@ represents the point of attachment to Ring W;
L¹ is -Cy^{L1}-; and
L² is -Cy^{L2}-.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-3 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, each -Cy- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently bivalent phenylenyl. In some embodiments, each -Cy- is independently an 8-10 membered bicyclic arylenyl. In some embodiments, each -Cy- is independently a 4-7 membered saturated or partially unsaturated carbocyclylenyl. In some embodiments, each -Cy- is independently a 6-11 membered saturated or partially unsaturated spiro carbocyclylenyl. In some embodiments, each -Cy- is independently an 8-10 membered bicyclic saturated or partially unsaturated carbocyclylenyl. In some embodiments, each -Cy- is independently a 4-7 membered saturated or partially unsaturated heterocyclylenyl having 1-2 heteroatoms independently selected from nitrogen, oxygen, and sulfur. In some embodiments, each -Cy- is independently a 6-11 membered saturated or partially unsaturated spiro heterocyclylenyl having 1-3 heteroatoms independently selected from nitrogen, oxygen, and sulfur. In some embodiments, each -Cy- is independently an 8-10 membered bicyclic saturated or partially unsaturated heterocyclylenyl having 1-2 heteroatoms independently selected from nitrogen, oxygen, and sulfur. In some embodiments, each -Cy- is independently a 5-6 membered heteroarylenyl having 1-4 heteroatoms independently selected from nitrogen, oxygen, and sulfur. In some embodiments, each -Cy- is independently an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy- is substituted with halogen. In some embodiments, -Cy- is substituted with —CN. In some embodiments, -Cy- is substituted with C₁₋₆ alkyl (e.g., methyl, ethyl, isopropyl). In some embodiments, -Cy- is substituted with C₃₋₆ cycloalkyl. In some embodiments, -Cy- is substituted with C₁₋₆ haloalkyl (e.g., —CHF₂, —CF₃). In some embodiments, -Cy- is substituted with —OC₁₋₆ alkyl (e.g., —OMe, —OEt). In some embodiments, -Cy- is substituted with —OC₁₋₆ haloalkyl (e.g., —OCHF₂, —OCF₃).

In some embodiments, -Cy- is substituted with methyl. In some embodiments, -Cy- is substituted with ethyl. In some embodiments, -Cy- is substituted with cyclopropyl. In some embodiments, -Cy- is substituted with —CHF₂. In some embodiments, -Cy- is substituted with —CMeF₂. In some embodiments, -Cy- is substituted with —CF₃. In some embodiments, -Cy- is substituted with —OCHF₂. In some embodiments, -Cy- is substituted with —OCMeF₂. In some embodiments, -Cy- is substituted with —OCF₃. In some embodiments, -Cy- is substituted with —C(Me)OH. In some embodiments, -Cy- is substituted with oxo. In some embodiments, -Cy- is substituted with fluoro. In some embodiments, -Cy- is substituted with geminal difluoro. In some embodiments, -Cy- is substituted with —OH. In some embodiments, -Cy- is substituted with —OMe. In some embodiments, -Cy- is substituted with —OEt. In some embodiments, -Cy- is substituted with —NR₂.

In some embodiments, -Cy- is phenylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is phenylenyl, optionally substituted with halogen (e.g., fluoro or chloro) or C₁₋₆ aliphatic.

In some embodiments, -Cy- is an optionally substituted 5 membered saturated or partially unsaturated heterocyclylenyl having 1-2 heteroatoms independently selected from nitrogen, oxygen, and sulfur. In some embodiments, -Cy- is an optionally substituted pyrrolidinylenyl. In some embodiments, -Cy- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrrolidinylenyl, optionally substituted with halogen (e.g., fluoro or chloro) or C₁₋₆ aliphatic. In some embodiments, -Cy^{L4}- is an optionally substituted 6 membered saturated or partially unsaturated heterocyclylenyl having 1-2 heteroatoms independently selected from nitrogen, oxygen, and sulfur. In some embodiments, -Cy- is an optionally substituted piperadinylenyl. In some embodiments, -Cy- is piperadinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is piperadinylenyl, optionally substituted with halogen (e.g., fluoro or chloro) or C₁₋₆ aliphatic. In some embodiments, -Cy- is an optionally substituted piperazinylenyl. In some embodiments, -Cy- is piperazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is piperazinylenyl, optionally substituted with halogen (e.g., fluoro or chloro) or C₁₋₃ aliphatic.

In some embodiments, -Cy- is an optionally substituted 5 membered heteroarylenyl having 1-4 heteroatoms independently selected from nitrogen, oxygen. In some embodiments, -Cy- is an optionally substituted pyrrolylenyl, pyrazolylenyl, imidazolylenyl, or triazolylenyl. In some embodiments, -Cy- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy- is an optionally substituted pyrazolylenyl. In some embodiments, -Cy- is pyrazolylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyrazolylenyl, optionally substituted with halogen (e.g., fluoro or chloro) or C₁₋₆ aliphatic. In some embodiments, -Cy- is an optionally substituted imidazolylenyl. In some embodiments, -Cy- is imidazolylenyl, optionally substituted with halogen (e.g., fluoro or chloro), —CN, C₁₋₆ aliphatic optionally further substituted with halogen (e.g., fluoro or chloro), or —OR^{o}, wherein R^{o} is hydrogen or C₁₋₆ aliphatic. In some embodiments, -Cy- is imidazolylenyl, optionally substituted with halogen (e.g., fluoro or chloro) or C₁₋₆ aliphatic.

In some embodiments, -Cy- is an optionally substituted 6 membered heteroarylenyl having 1-4 heteroatoms independently selected from nitrogen, oxygen. In some embodiments, -Cy- is an optionally substituted pyridinylenyl, pyridazinylenyl, pyrimidinylenyl, pyrazinylenyl, or triazinylenyl. In some embodiments, -Cy- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy- is an optionally substituted pyridinylenyl. In some embodiments, -Cy- is pyridinylenyl, optionally substituted with halogen (e.g., fluoro or chloro), —CN, C₆ aliphatic optionally further substituted with halogen (e.g., fluoro or chloro), —S(O)₂R^{o}, or —OR^{o} wherein R^{o} is hydrogen C₁₋₆ aliphatic. In some embodiments, -Cy- is pyridinylenyl, optionally substituted with halogen (e.g., fluoro or chloro) or C₁₋₆ aliphatic. In some embodiments, -Cy- is an optionally substituted pyridazinylenyl. In some embodiments, -Cy- is pyridazinylenvl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₃ aliphatic. In some embodiments, -Cy- is pyridazinylenyl, optionally substituted with halogen (e.g., fluoro or chloro) or C₁₋₆ aliphatic. In some embodiments, -Cy- is an optionally substituted pyridinonylenyl, pyridazinonylenyl, pyrimidinonylenyl, pyrazinonylenyl, or triazinonylenyl. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁₋₆ aliphatic. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy- is an optionally substituted pyridinonylenyl. In some embodiments, -Cy- is pyridinonylenyl, optionally substituted with halogen (e.g., fluoro or chloro), —CN, C₁₋₆ aliphatic optionally further substituted with halogen (e.g., fluoro or chloro), —S(O)₂R^{o}, or —OR^{o}, wherein R^{o} is hydrogen or C₁s aliphatic. In some embodiments, -Cy- is pyridinonylenyl, optionally substituted with halogen (e.g., fluoro or chloro) or C₁₋₆ aliphatic.

In some embodiments, -Cy- is an optionally substituted cyclopropyl. In some embodiments, -Cy^{L1}- is an optionally substituted 5 membered saturated or partially unsaturated carbocyclylenyl. In some embodiments, -Cy- is an optionally substituted cyclopentanylenyl or cyclopentenylenyl. In some embodiments, -Cy- is an optionally substituted 6 membered saturated or partially unsaturated carbocyclylenyl. In some embodiments, -Cy- is an optionally substituted cyclohexanylenyl or cyclohexenylenyl. In some embodiments, -Cy- is an optionally substituted naphthalenylenyl.

In some embodiments, -Cy- is an optionally substituted 9 membered bicyclic heteroarylenyl having 1-5 heteroatoms independently selected from nitrogen, oxygen, and sulfur. In some embodiments, -Cy- is an optionally substituted indolylenyl, azaindolylenyl, isoindolylenyl, azaisoindolylenyl, indazolylenyl, azaindazolylenyl, benzimidazolinyl, or azabenzimidazolylenyl. In some embodiments, -Cy- is an optionally substituted indolylenyl. In some embodiments, -Cy- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy- is an optionally substituted 9-11 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 nitrogen heteroatoms. In some embodiments, -Cy- is an optionally substituted 4,6-spiro heterocyclylenyl having 1-3 heteroatoms independently selected from nitrogen, oxygen, and sulfur. In some embodiments, -Cy- is an optionally substituted

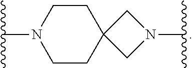 [SMILES: *N1CCC2(CC1)CN(*)C2]

In some embodiments, -Cy- is an optionally substituted 10 membered saturated or partially unsaturated spiro heterocyclylenyl having 1-3 heteroatoms independently selected from nitrogen, oxygen, and sulfur. In some embodiments, -Cy- is an optionally substituted 10 membered saturated or partially unsaturated spiro heterocyclylenyl having 1-3 nitrogen heteroatoms. In some embodiments, -Cy- is an optionally substituted 5,6-spiro heterocyclylenyl having 1-3 heteroatoms independently selected from nitrogen, oxygen, and sulfur.

In some embodiments, -Cy- is an optionally substituted 11 membered saturated or partially unsaturated spiro heterocyclylenyl having 1-3 heteroatoms independently selected from nitrogen, oxygen, and sulfur. In some embodiments, -Cy- is an optionally substituted 11 membered saturated or partially unsaturated spiro heterocyclylenyl having 1-3 nitrogen heteroatoms. In some embodiments, -Cy- is an optionally substituted 6,6-spiro heterocyclylenyl having 1-3 heteroatoms independently selected from nitrogen, oxygen, and sulfur.

In some embodiments, -Cy- is

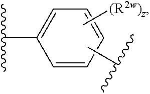

wherein R^{2w} and z are defined above and as described herein.

In some embodiments, -Cy- is

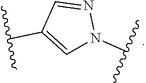 [SMILES: *c1cnn(*)c1]

In some embodiments, -Cy- is

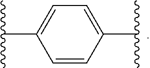 [SMILES: *c1ccc(*)cc1]

In some embodiments, -Cy- is

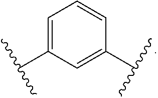 [SMILES: *c1cccc(*)c1]

In some embodiments, -Cy- is

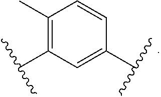 [SMILES: *c1ccc(C)c(*)c1]

In some embodiments, -Cy- is

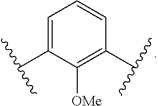 [SMILES: *c1cccc(*)c1OC]

In some embodiments, -Cy- is

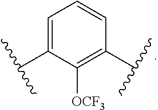 [SMILES: *c1cccc(*)c1OC(F)(F)F]

In some embodiments, -Cy- is

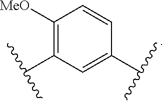 [SMILES: *c1ccc(OC)c(*)c1]

In some embodiments, -Cy- is

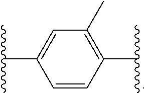 [SMILES: *c1ccc(*)c(C)c1]

In some embodiments, -Cy- is

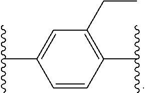 [SMILES: *c1ccc(*)c(CC)c1]

In some embodiments, -Cy- is

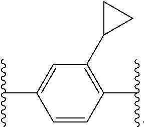 [SMILES: *c1ccc(*)c(C2CC2)c1]

In some embodiments, -Cy- is

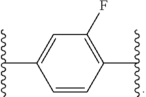 [SMILES: *c1ccc(*)c(F)c1]

In some embodiments, -Cy- is

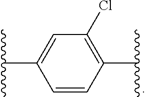 [SMILES: *c1ccc(*)c(Cl)c1]

In some embodiments, -Cy- is

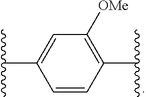 [SMILES: *c1ccc(*)c(OC)c1]

In some embodiments, -Cy- is

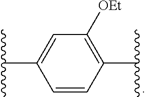 [SMILES: *c1ccc(*)c(OCC)c1]

In some embodiments, -Cy- is

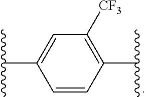 [SMILES: *c1ccc(*)c(C(F)(F)F)c1]

In some embodiments, -Cy- is

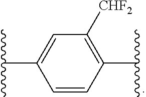 [SMILES: *c1ccc(*)c(C(F)F)c1]

In some embodiments, -Cy- is

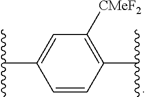

In some embodiments, -Cy- is

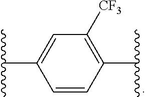 [SMILES: *c1ccc(*)c(C(F)(F)F)c1]

In some embodiments, -Cy- is

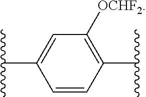 [SMILES: *c1ccc(*)c(OC(F)F)c1]

In some embodiments, -Cy- is

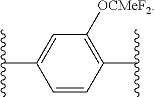

In some embodiments, -Cy- is

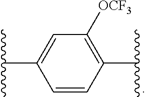 [SMILES: *c1ccc(*)c(OC(F)(F)F)c1]

In some embodiments, -Cy- is

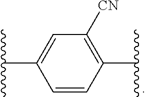 [SMILES: *c1ccc(*)c(C#N)c1]

In some embodiments, -Cy- is

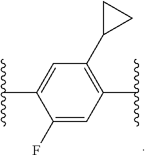 [SMILES: *c1cc(C2CC2)c(*)cc1F]

In some embodiments, -Cy- is

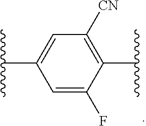 [SMILES: *c1cc(F)c(*)c(C#N)c1]

In some embodiments, -Cy- is

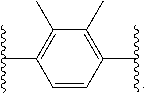 [SMILES: *c1ccc(*)c(C)c1C]

In some embodiments, -Cy- is

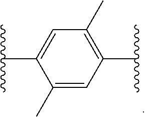 [SMILES: *c1cc(C)c(*)cc1C]

In some embodiments, -Cy- is

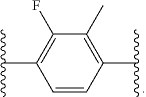 [SMILES: *c1ccc(*)c(F)c1C]

In some embodiments, -Cy- is

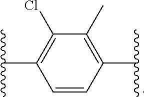 [SMILES: *c1ccc(*)c(Cl)c1C]

In some embodiments, -Cy- is

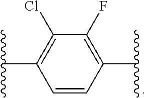 [SMILES: *c1ccc(*)c(Cl)c1F]

In some embodiments, -Cy- is

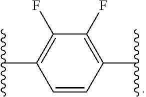 [SMILES: *c1ccc(*)c(F)c1F]

In some embodiments, -Cy- is

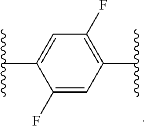 [SMILES: *c1cc(F)c(*)cc1F]

In some embodiments, -Cy- is

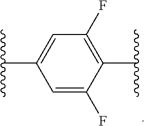 [SMILES: *c1cc(F)c(*)c(F)c1]

In some embodiments, -Cy- is

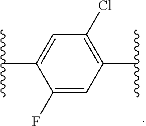 [SMILES: *c1cc(Cl)c(*)cc1F]

In some embodiments, -Cy- is

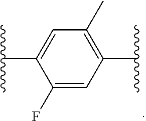 [SMILES: *c1cc(F)c(*)cc1C]

In some embodiments, -Cy- is

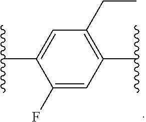 [SMILES: *c1cc(CC)c(*)cc1F]

In some embodiments, -Cy- is

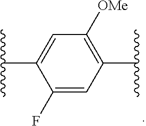 [SMILES: *c1cc(OC)c(*)cc1F]

In some embodiments, -Cy- is

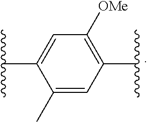 [SMILES: *c1cc(OC)c(*)cc1C]

In some embodiments, -Cy- is

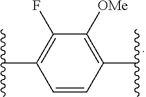 [SMILES: *c1ccc(*)c(OC)c1F]

In some embodiments, -Cy- is

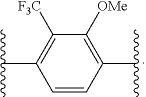 [SMILES: *c1ccc(*)c(C(F)(F)F)c1OC]

In some embodiments, -Cy- is

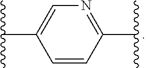 [SMILES: *c1ccc(*)nc1]

In some embodiments, -Cy- is

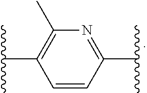 [SMILES: *c1ccc(*)c(C)n1 ; "."]

In some embodiments, -Cy- is

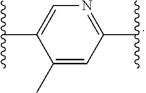 [SMILES: *c1cc(C)c(*)cn1]

In some embodiments, -Cy- is

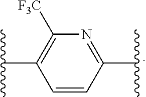 [SMILES: *c1ccc(*)c(C(F)(F)F)n1]

In some embodiments, -Cy- is

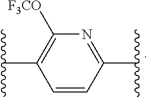 [SMILES: *c1ccc(*)c(OC(F)(F)F)n1]

In some embodiments, -Cy- is

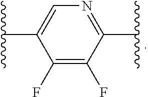 [SMILES: *c1cnc(*)c(F)c1F]

In some embodiments, -Cy- is

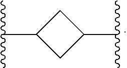 [SMILES: *C1CC(*)C1]

In some embodiments, -Cy- is

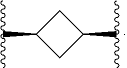 [SMILES: *[C@H]1C[C@@H](*)C1]

In some embodiments, -Cy- is

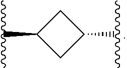 [SMILES: *[C@H]1C[C@H](*)C1]

In some embodiments, -Cy- is

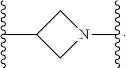 [SMILES: *C1CN(*)C1]

In some embodiments, -Cy- is

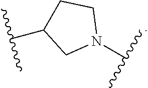 [SMILES: *C1CCN(*)C1]

In some embodiments, -Cy- is

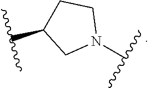 [SMILES: *[C@H]1CCN(*)C1]

In some embodiments, -Cy- is

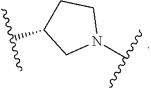 [SMILES: *[C@@H]1CCN(*)C1]

In some embodiments, -Cy- is

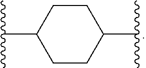 [SMILES: *C1CCC(*)CC1]

In some embodiments, -Cy- is

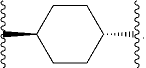 [SMILES: *[C@H]1CC[C@H](*)CC1]

In some embodiments, -Cy- is

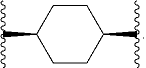 [SMILES: *[C@H]1CC[C@@H](*)CC1]

In some embodiments, -Cy- is

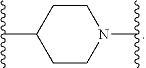 [SMILES: *C1CCN(*)CC1]

In some embodiments, -Cy- is

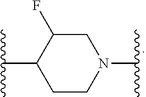 [SMILES: *C1CCN(*)CC1F]

In some embodiments, -Cy- is

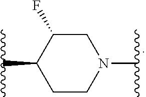 [SMILES: *[C@@H]1CCN(*)C[C@H]1F]

In some embodiments, -Cy- is

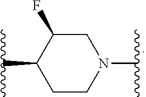 [SMILES: *[C@@H]1CCN(*)C[C@@H]1F]

In some embodiments, -Cy- is

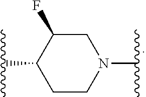 [SMILES: *[C@H]1CCN(*)C[C@@H]1F]

In some embodiments, -Cy- is

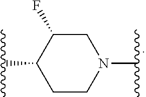 [SMILES: *[C@H]1CCN(*)C[C@H]1F]

In some embodiments, -Cy- is

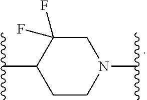 [SMILES: *C1CCN(*)CC1(F)F]

In some embodiments, -Cy- is

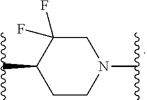 [SMILES: *[C@@H]1CCN(*)CC1(F)F]

In some embodiments, -Cy- is

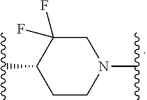 [SMILES: *[C@H]1CCN(*)CC1(F)F]

In some embodiments, -Cy- is

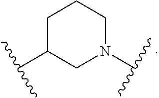 [SMILES: *C1CCCN(*)C1]

In some embodiments, -Cy- is

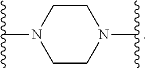 [SMILES: *N1CCN(*)CC1]

In some embodiments, -Cy- is

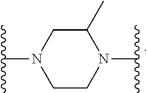 [SMILES: *N1CCN(*)C(C)C1]

In some embodiments, -Cy- is

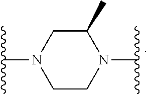 [SMILES: *N1CCN(*)[C@H](C)C1]

In some embodiments, -Cy- is

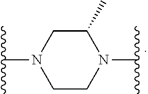 [SMILES: *N1CCN(*)[C@@H](C)C1]

In some embodiments, -Cy- is

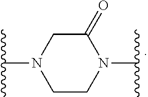 [SMILES: *N1CCN(*)C(=O)C1]

In some embodiments, -Cy- is

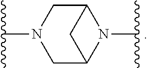 [SMILES: *N1CC2CC(C1)N2*]

In some embodiments, -Cy- is

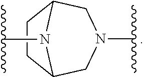 [SMILES: *N1CC2CCC(C1)N2*]

In some embodiments, -Cy- is

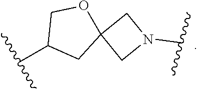 [SMILES: *C1COC2(C1)CN(*)C2]

In some embodiments, -Cy- is

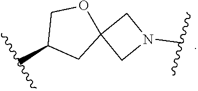 [SMILES: *[C@H]1COC2(C1)CN(*)C2]

In some embodiments, -Cy- is

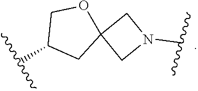 [SMILES: *[C@@H]1COC2(C1)CN(*)C2]

In some embodiments, -Cy- is

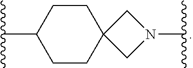 [SMILES: *C1CCC2(CC1)CN(*)C2]

In some embodiments, -Cy- is

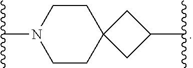 [SMILES: *C1CC2(CCN(*)CC2)C1]

In some embodiments, -Cy- is

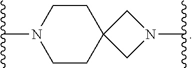 [SMILES: *N1CCC2(CC1)CN(*)C2]

In some embodiments, -Cy- is

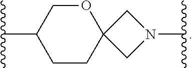 [SMILES: *C1CCC2(CN(*)C2)OC1]

In some embodiments, -Cy- is

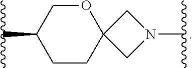 [SMILES: *[C@@H]1CCC2(CN(*)C2)OC1]

In some embodiments, -Cy- is

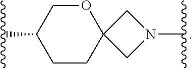 [SMILES: *[C@H]1CCC2(CN(*)C2)OC1]

In some embodiments, -Cy- is

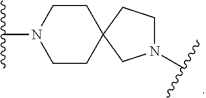 [SMILES: *N1CCC2(CC1)CCN(*)C2]

In some embodiments, -Cy- is

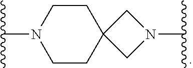 [SMILES: *N1CCC2(CC1)CN(*)C2]

In some embodiments, -Cy- is

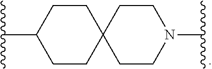 [SMILES: *C1CCC2(CC1)CCN(*)CC2]

In some embodiments, -Cy- is

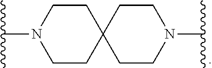 [SMILES: *N1CCC2(CC1)CCN(*)CC2]

In some embodiments, -Cy- is

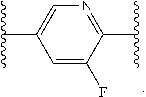 [SMILES: *c1cnc(*)c(F)c1]

In some embodiments, -Cy- is selected from those depicted in Table 1B, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1B, below.

In some embodiments, L is

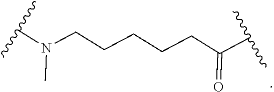 [SMILES: *C(=O)CCCCCN(*)C]

In some embodiments, L is

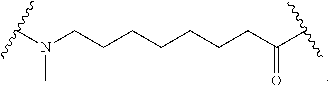 [SMILES: *C(=O)CCCCCCCN(*)C]

In some embodiments, L is

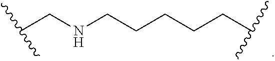 [SMILES: *CCCCCNC*]

In some embodiments, L is

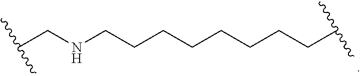 [SMILES: *CCCCCCCCNC*]

In some embodiments, L is

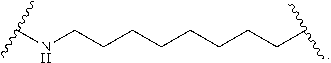 [SMILES: *CCCCCCCCN*]

In some embodiments, L is

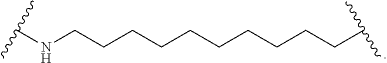 [SMILES: *CCCCCCCCCCN*]

In some embodiments, L is

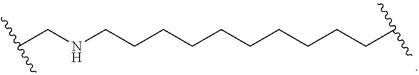 [SMILES: *CCCCCCCCCCNC*]

In some embodiments, L is

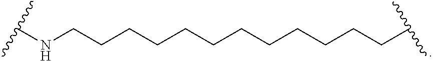 [SMILES: *CCCCCCCCCCCCN*]

In some embodiments, L is

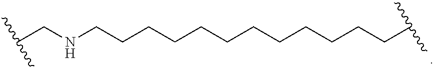 [SMILES: *CCCCCCCCCCCCNC*]

In some embodiments, L is

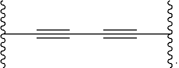 [SMILES: *C#CC#C*]

In some embodiments, L is

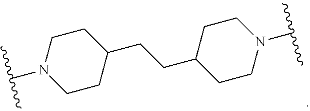 [SMILES: *N1CCC(CCC2CCN(*)CC2)CC1]

In some embodiments, L is

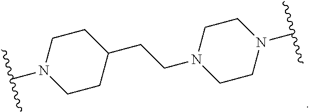 [SMILES: *N1CCC(CCN2CCN(*)CC2)CC1]

In some embodiments, L is

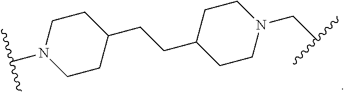 [SMILES: *CN1CCC(CCC2CCN(*)CC2)CC1]

In some embodiments, L is

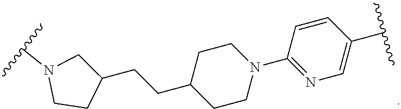 [SMILES: *c1ccc(N2CCC(CCC3CCN(*)C3)CC2)nc1]

In some embodiments, L is

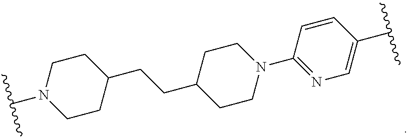 [SMILES: *c1ccc(N2CCC(CCC3CCN(*)CC3)CC2)nc1]

In some embodiments, L is

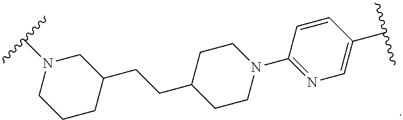 [SMILES: *c1ccc(N2CCC(CCC3CCCN(*)C3)CC2)nc1]

In some embodiments, L is

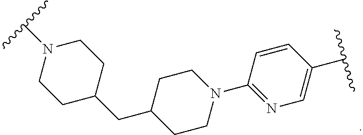 [SMILES: *c1ccc(N2CCC(CC3CCN(*)CC3)CC2)nc1]

In some embodiments, L is

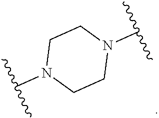 [SMILES: *N1CCN(*)CC1]

In some embodiments, L is

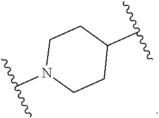 [SMILES: *C1CCN(*)CC1]

In some embodiments, L is

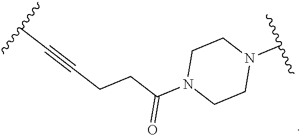 [SMILES: *C#CCCC(=O)N1CCN(*)CC1]

In some embodiments, L is

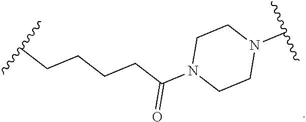 [SMILES: *CCCCC(=O)N1CCN(*)CC1]

In some embodiments, L is

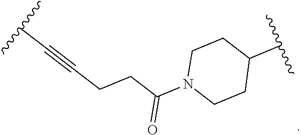 [SMILES: *C#CCCC(=O)N1CCC(*)CC1]

In some embodiments, L is

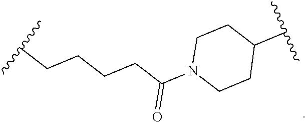 [SMILES: *CCCCC(=O)N1CCC(*)CC1]

In some embodiments, L is

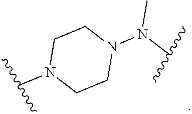 [SMILES: *N1CCN(N(*)C)CC1]

In some embodiments, L is

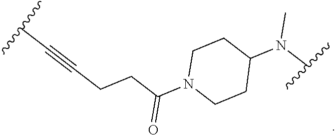 [SMILES: *C#CCCC(=O)N1CCC(N(*)C)CC1]

In some embodiments, L is

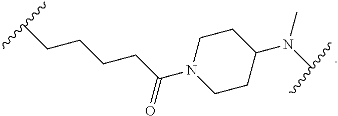 [SMILES: *CCCCC(=O)N1CCC(N(*)C)CC1]

In some embodiments, L is

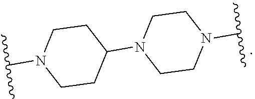 [SMILES: *N1CCC(N2CCN(*)CC2)CC1]

In some embodiments, L is

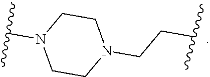 [SMILES: *CCN1CCN(*)CC1]

In some embodiments, L is

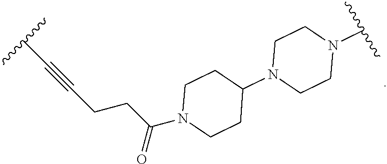 [SMILES: *C#CCCC(=O)N1CCC(N2CCN(*)CC2)CC1]

In some embodiments, L is

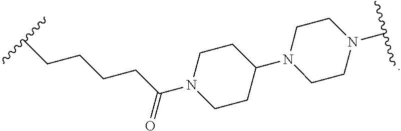 [SMILES: *CCCCC(=O)N1CCC(N2CCN(*)CC2)CC1]

In some embodiments, L is

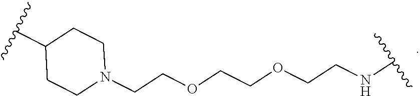 [SMILES: *NCCOCCOCCN1CCC(*)CC1]

In some embodiments, L is

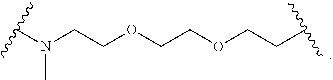 [SMILES: *CCOCCOCCN(*)C]

In some embodiments, L is

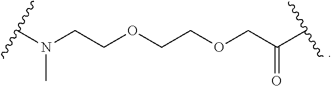 [SMILES: *C(=O)COCCOCCN(*)C]

In some embodiments, L is

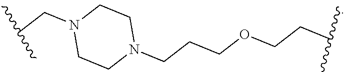 [SMILES: *CCOCCCN1CCN(C*)CC1]

In some embodiments, L is

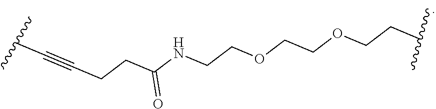 [SMILES: *C#CCCC(=O)NCCOCCOCC*]

In some embodiments, L is

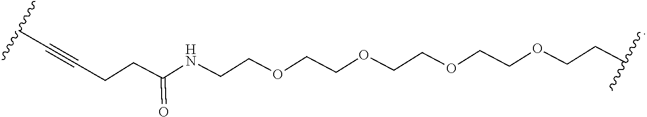 [SMILES: *C#CCCC(=O)NCCOCCOCCOCCOCC*]

In some embodiments, L is

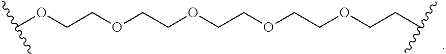 [SMILES: *CCOCCOCCOCCOCCO*]

In some embodiments, L is

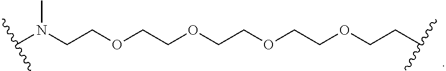 [SMILES: *CCOCCOCCOCCOCCN(*)C]

In some embodiments, L is

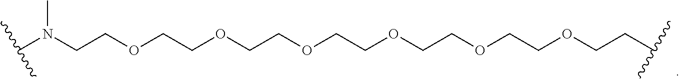 [SMILES: *CCOCCOCCOCCOCCOCCOCCN(*)C]

In some embodiments, L is

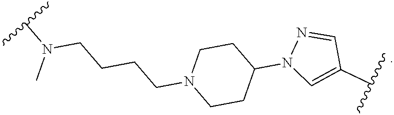 [SMILES: *c1cnn(C2CCN(CCCCN(*)C)CC2)c1]

In some embodiments, L is

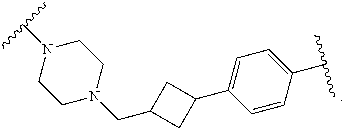 [SMILES: *c1ccc(C2CC(CN3CCN(*)CC3)C2)cc1]

In some embodiments, L is

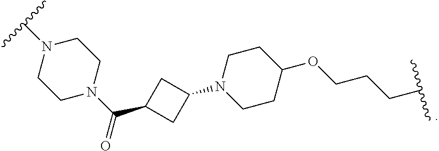 [SMILES: *CCCOC1CCN([C@H]2C[C@H](C(=O)N3CCN(*)CC3)C2)CC1]

In some embodiments, L is

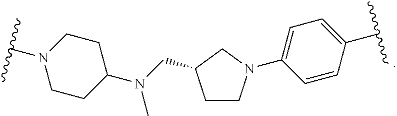 [SMILES: *c1ccc(N2CC[C@H](CN(C)C3CCN(*)CC3)C2)cc1]

In some embodiments, L is

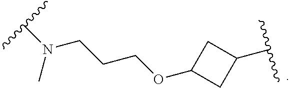 [SMILES: *C1CC(OCCCN(*)C)C1]

In some embodiments, L is

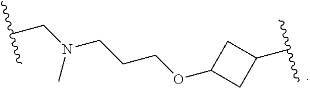 [SMILES: *CN(C)CCCOC1CC(*)C1]

In some embodiments, L is

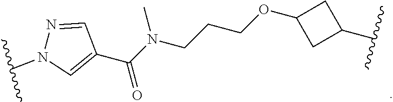 [SMILES: *C1CC(OCCCN(C)C(=O)c2cnn(*)c2)C1]

In some embodiments, L is

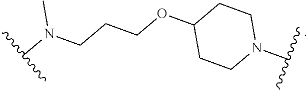 [SMILES: *N(C)CCCOC1CCN(*)CC1]

In some embodiments, L is

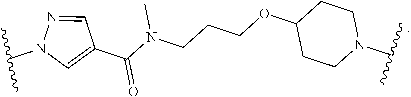 [SMILES: *N1CCC(OCCCN(C)C(=O)c2cnn(*)c2)CC1]

In some embodiments, L is

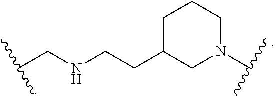 [SMILES: *CNCCC1CCCN(*)C1]

In some embodiments, L is

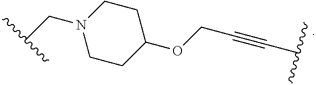 [SMILES: *C#CCOC1CCN(C*)CC1]

In some embodiments, L is

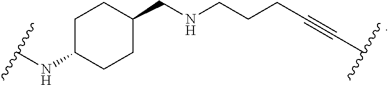 [SMILES: *C#CCCCNC[C@H]1CC[C@H](N*)CC1]

In some embodiments, L is

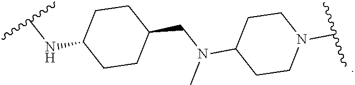 [SMILES: *N[C@H]1CC[C@H](CN(C)C2CCN(*)CC2)CC1]

In some embodiments, L is

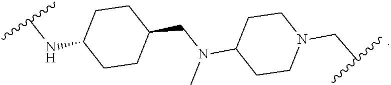 [SMILES: *CN1CCC(N(C)C[C@H]2CC[C@H](N*)CC2)CC1]

In some embodiments, L is

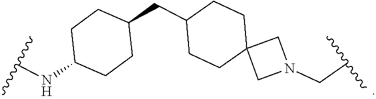 [SMILES: *CN1CC2(CCC(C[C@H]3CC[C@H](N*)CC3)CC2)C1]

In some embodiments, L is

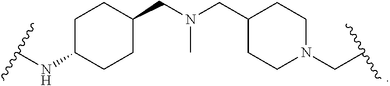 [SMILES: *CN1CCC(CN(C)C[C@H]2CC[C@H](N*)CC2)CC1]

In some embodiments, L is

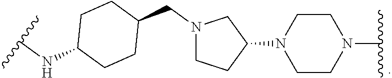 [SMILES: *N[C@H]1CC[C@H](CN2CC[C@@H](N3CCN(*)CC3)C2)CC1]

In some embodiments, L is

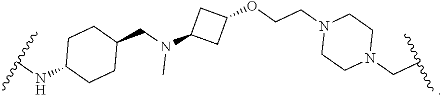 [SMILES: *CN1CCN(CCO[C@H]2C[C@H](N(C)C[C@H]3CC[C@H](N*)CC3)C2)CC1]

In some embodiments, L is

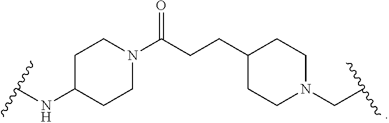 [SMILES: *CN1CCC(CCC(=O)N2CCC(N*)CC2)CC1]

In some embodiments, L is

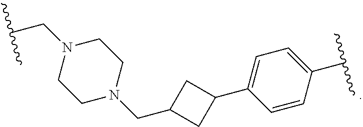 [SMILES: *CN1CCN(CC2CC(c3ccc(*)cc3)C2)CC1]

In some embodiments, L is

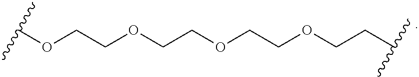 [SMILES: *CCOCCOCCOCCO*]

In some embodiments, L is

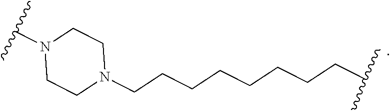 [SMILES: *CCCCCCCCN1CCN(*)CC1]

In some embodiments, L is

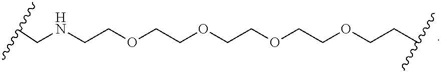 [SMILES: *CCOCCOCCOCCOCCNC*]

In some embodiments, L is

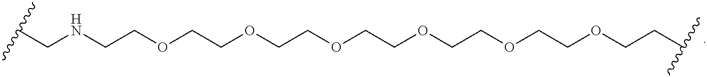 [SMILES: *CCOCCOCCOCCOCCOCCOCCNC*]

In some embodiments, L is

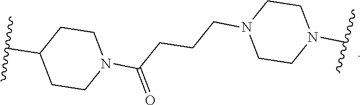 [SMILES: *C1CCN(C(=O)CCCN2CCN(*)CC2)CC1]

In some embodiments, L is

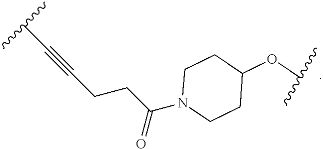 [SMILES: *C#CCCC(=O)N1CCC(O*)CC1]

In some embodiments, L is

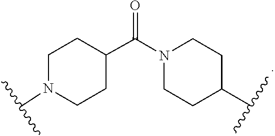 [SMILES: *C1CCN(C(=O)C2CCN(*)CC2)CC1]

In some embodiments, L is

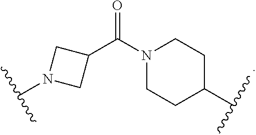 [SMILES: *C1CCN(C(=O)C2CN(*)C2)CC1]

In some embodiments, L is

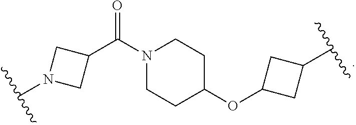 [SMILES: *C1CC(OC2CCN(C(=O)C3CN(*)C3)CC2)C1]

In some embodiments, L is

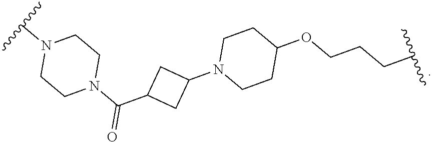 [SMILES: *CCCOC1CCN(C2CC(C(=O)N3CCN(*)CC3)C2)CC1]

In some embodiments, L is

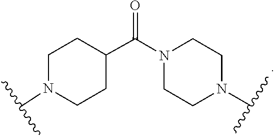 [SMILES: *N1CCC(C(=O)N2CCN(*)CC2)CC1]

In some embodiments, L is

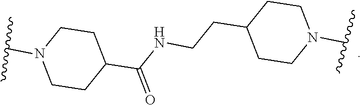 [SMILES: *N1CCC(CCNC(=O)C2CCN(*)CC2)CC1]

In some embodiments, L is

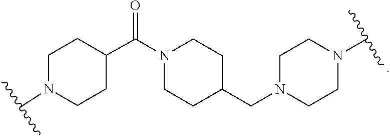 [SMILES: *N1CCC(C(=O)N2CCC(CN3CCN(*)CC3)CC2)CC1]

In some embodiments, L is

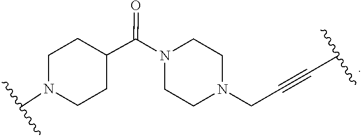 [SMILES: *C#CCN1CCN(C(=O)C2CCN(*)CC2)CC1]

In some embodiments, L is

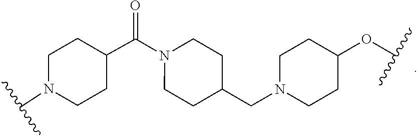 [SMILES: *OC1CCN(CC2CCN(C(=O)C3CCN(*)CC3)CC2)CC1]

In some embodiments, L is

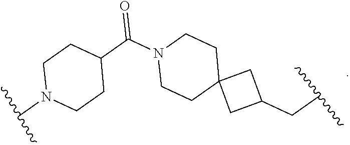 [SMILES: *CC1CC2(CCN(C(=O)C3CCN(*)CC3)CC2)C1]

In some embodiments, L is

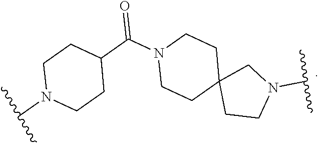 [SMILES: *N1CCC(C(=O)N2CCC3(CCN(*)C3)CC2)CC1]

In some embodiments, L is

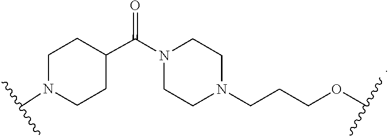 [SMILES: *OCCCN1CCN(C(=O)C2CCN(*)CC2)CC1]

In some embodiments, L is

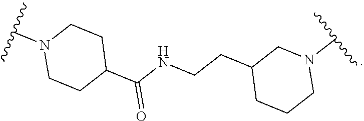 [SMILES: *N1CCC(C(=O)NCCC2CCCN(*)C2)CC1]

In some embodiments, L is

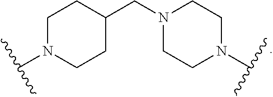 [SMILES: *N1CCC(CN2CCN(*)CC2)CC1]

In some embodiments, L is

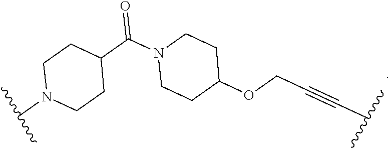 [SMILES: *C#CCOC1CCN(C(=O)C2CCN(*)CC2)CC1]

In some embodiments, L is

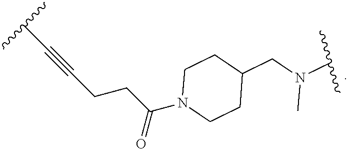 [SMILES: *C#CCCC(=O)N1CCC(CN(*)C)CC1]

In some embodiments, L is

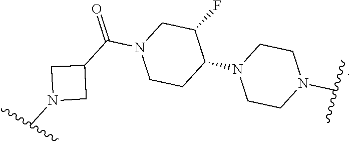 [SMILES: *N1CCN([C@@H]2CCN(C(=O)C3CN(*)C3)C[C@@H]2F)CC1]

In some embodiments, L is

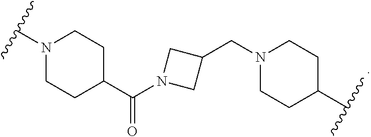 [SMILES: *C1CCN(CC2CN(C(=O)C3CCN(*)CC3)C2)CC1]

In some embodiments, L is

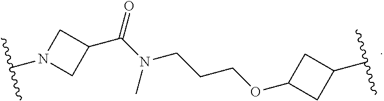 [SMILES: *C1CC(OCCCN(C)C(=O)C2CN(*)C2)C1]

In some embodiments, L is

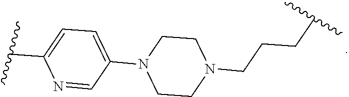 [SMILES: *CCCN1CCN(c2ccc(*)nc2)CC1]

In some embodiments, L is

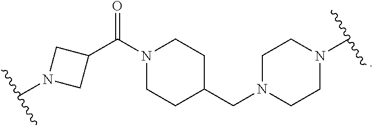 [SMILES: *N1CCN(CC2CCN(C(=O)C3CN(*)C3)CC2)CC1]

In some embodiments, L is

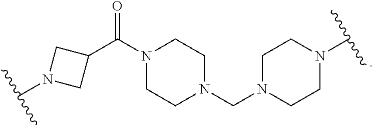 [SMILES: *N1CCN(CN2CCN(C(=O)C3CN(*)C3)CC2)CC1]

In some embodiments, L is

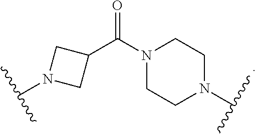 [SMILES: *N1CCN(C(=O)C2CN(*)C2)CC1]

In some embodiments, L is

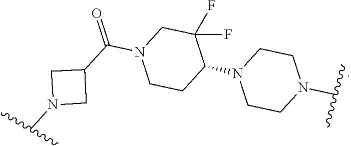 [SMILES: *N1CCN([C@@H]2CCN(C(=O)C3CN(*)C3)CC2(F)F)CC1]

In some embodiments, L is

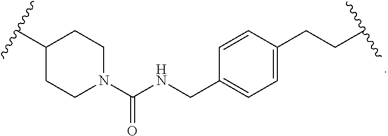 [SMILES: *CCc1ccc(CNC(=O)N2CCC(*)CC2)cc1]

In some embodiments, L is

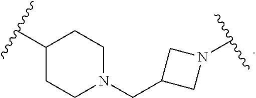 [SMILES: *C1CCN(CC2CN(*)C2)CC1]

In some embodiments, L is

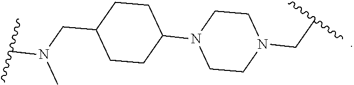 [SMILES: *CN1CCN(C2CCC(CN(*)C)CC2)CC1]

In some embodiments, L is

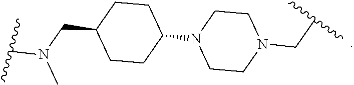 [SMILES: *CN1CCN([C@H]2CC[C@H](CN(*)C)CC2)CC1]

In some embodiments, L is

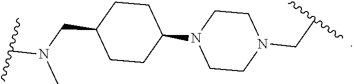 [SMILES: *CN1CCN([C@H]2CC[C@@H](CN(*)C)CC2)CC1]

In some embodiments, L is

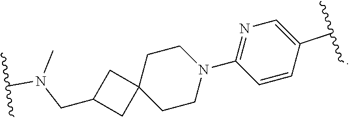 [SMILES: *c1ccc(N2CCC3(CC2)CC(CN(*)C)C3)nc1]

In some embodiments, L is

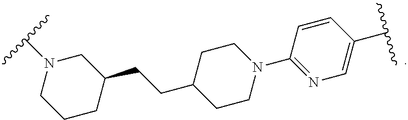 [SMILES: *c1ccc(N2CCC(CC[C@H]3CCCN(*)C3)CC2)nc1]

In some embodiments, L is

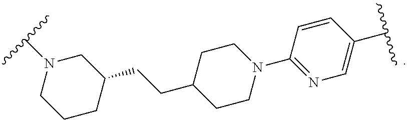 [SMILES: *c1ccc(N2CCC(CC[C@@H]3CCCN(*)C3)CC2)nc1]

In some embodiments, L is

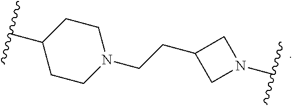 [SMILES: *C1CCN(CCC2CN(*)C2)CC1]

In some embodiments, L is

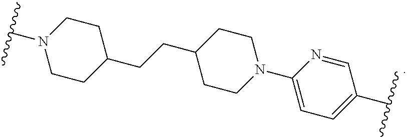 [SMILES: *c1ccc(N2CCC(CCC3CCN(*)CC3)CC2)nc1]

In some embodiments, L is

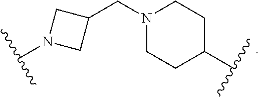 [SMILES: *C1CCN(CC2CN(*)C2)CC1]

In some embodiments, L is

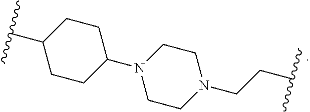 [SMILES: *CCN1CCN(C2CCC(*)CC2)CC1]

In some embodiments, L is

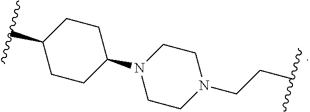 [SMILES: *CCN1CCN([C@H]2CC[C@@H](*)CC2)CC1]

In some embodiments, L is

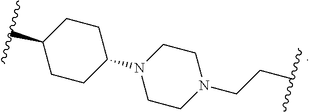 [SMILES: *CCN1CCN([C@H]2CC[C@H](*)CC2)CC1]

In some embodiments, L is

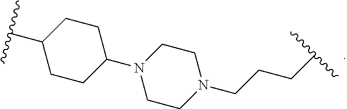 [SMILES: *CCCN1CCN(C2CCC(*)CC2)CC1]

In some embodiments, L is

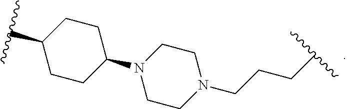 [SMILES: *CCCN1CCN([C@H]2CC[C@@H](*)CC2)CC1]

In some embodiments, L is

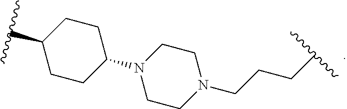 [SMILES: *CCCN1CCN([C@H]2CC[C@H](*)CC2)CC1]

In some embodiments, L is

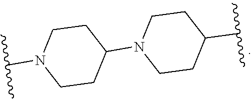 [SMILES: *C1CCN(C2CCN(*)CC2)CC1]

In some embodiments, L is

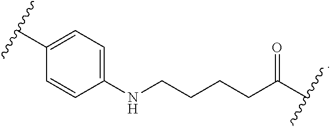 [SMILES: *C(=O)CCCCNc1ccc(*)cc1]

In some embodiments, L is

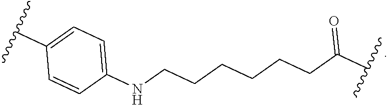 [SMILES: *C(=O)CCCCCCNc1ccc(*)cc1]

In some embodiments, L is

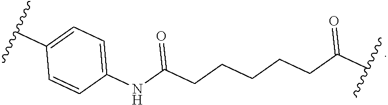 [SMILES: *C(=O)CCCCCC(=O)Nc1ccc(*)cc1]

In some embodiments, L is

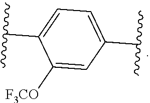 [SMILES: *c1ccc(*)c(OC(F)(F)F)c1]

In some embodiments, L is

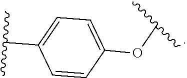 [SMILES: *Oc1ccc(*)cc1]

In some embodiments, L is

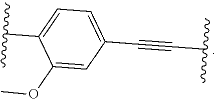 [SMILES: *C#Cc1ccc(*)c(OC)c1]

In some embodiments, L is

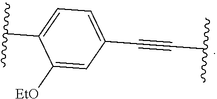 [SMILES: *C#Cc1ccc(*)c(OCC)c1]

In some embodiments, L is

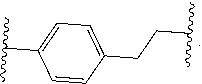 [SMILES: *CCc1ccc(*)cc1]

In some embodiments, L is

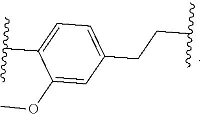 [SMILES: *CCc1ccc(*)c(OC)c1]

In some embodiments, L is

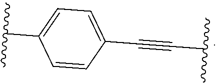 [SMILES: *C#Cc1ccc(*)cc1]

In some embodiments, L is

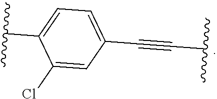 [SMILES: *C#Cc1ccc(*)c(Cl)c1]

In some embodiments, L is

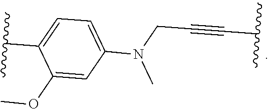 [SMILES: *C#CCN(C)c1ccc(*)c(OC)c1]

In some embodiments, L is

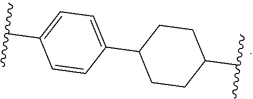 [SMILES: *c1ccc(C2CCC(*)CC2)cc1]

In some embodiments, L is

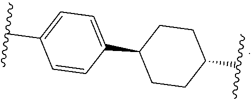 [SMILES: *c1ccc([C@H]2CC[C@H](*)CC2)cc1]

In some embodiments, L is

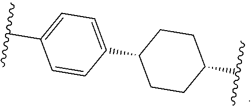 [SMILES: *c1ccc([C@H]2CC[C@@H](*)CC2)cc1]

In some embodiments, L is

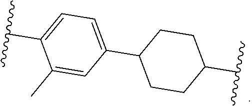 [SMILES: *c1ccc(C2CCC(*)CC2)cc1C]

In some embodiments, L is

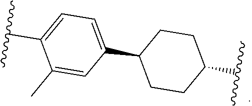 [SMILES: *c1ccc([C@H]2CC[C@H](*)CC2)cc1C]

In some embodiments, L is

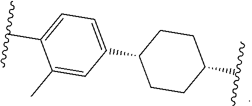 [SMILES: *c1ccc([C@H]2CC[C@@H](*)CC2)cc1C]

In some embodiments, L is

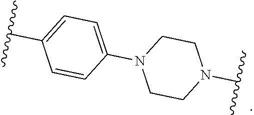 [SMILES: *c1ccc(N2CCN(*)CC2)cc1]

In some embodiments, L is

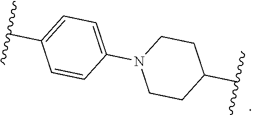 [SMILES: *c1ccc(N2CCC(*)CC2)cc1]

In some embodiments, L is

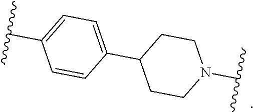 [SMILES: *c1ccc(C2CCN(*)CC2)cc1]

In some embodiments, L is

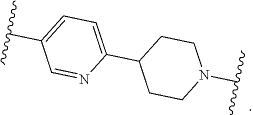 [SMILES: *c1ccc(C2CCN(*)CC2)nc1]

In some embodiments, L is

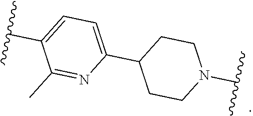 [SMILES: *c1ccc(C2CCN(*)CC2)nc1C]

In some embodiments, L is

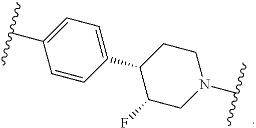 [SMILES: *c1ccc([C@@H]2CCN(*)C[C@@H]2F)cc1]

In some embodiments, L is

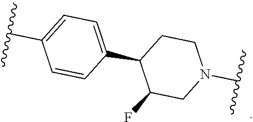 [SMILES: *c1ccc([C@H]2CCN(*)C[C@H]2F)cc1]

In some embodiments, L is

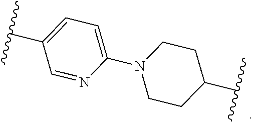 [SMILES: *c1ccc(N2CCC(*)CC2)nc1]

In some embodiments, L is

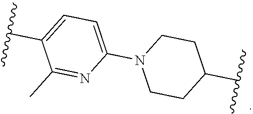 [SMILES: *c1ccc(N2CCC(*)CC2)nc1C]

In some embodiments, L is

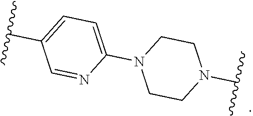 [SMILES: *c1ccc(N2CCN(*)CC2)nc1]

In some embodiments, L is

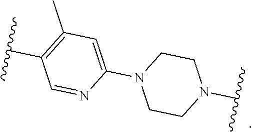 [SMILES: *c1cnc(N2CCN(*)CC2)cc1C]

In some embodiments, L is

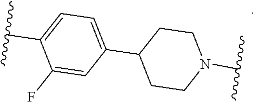 [SMILES: *c1ccc(C2CCN(*)CC2)cc1F]

In some embodiments, L is

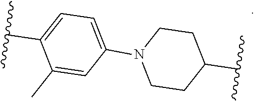 [SMILES: *c1ccc(N2CCC(*)CC2)cc1C]

In some embodiments, L is

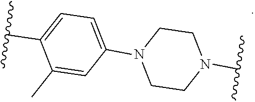 [SMILES: *c1ccc(N2CCN(*)CC2)cc1C]

In some embodiments, L is

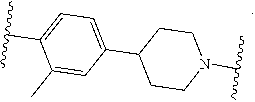 [SMILES: *c1ccc(C2CCN(*)CC2)cc1C]

In some embodiments, L is

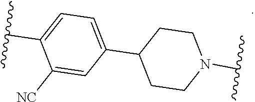 [SMILES: *c1ccc(C2CCN(*)CC2)cc1C#N]

In some embodiments, L is

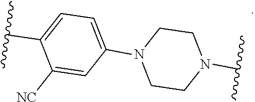 [SMILES: *c1ccc(N2CCN(*)CC2)cc1C#N]

In some embodiments, L is

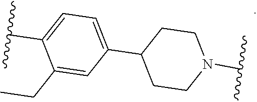 [SMILES: *c1ccc(C2CCN(*)CC2)cc1CC]

In some embodiments, L is

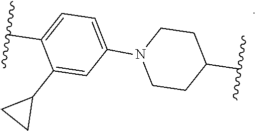 [SMILES: *c1ccc(N2CCC(*)CC2)cc1C1CC1]

In some embodiments, L is

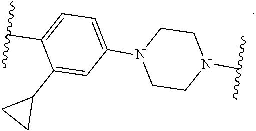 [SMILES: *c1ccc(N2CCN(*)CC2)cc1C1CC1]

In some embodiments, L is

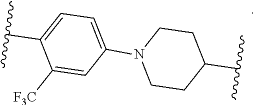 [SMILES: *c1ccc(N2CCC(*)CC2)cc1C(F)(F)F]

In some embodiments, L is

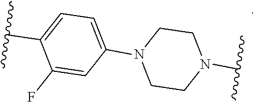 [SMILES: *c1ccc(N2CCN(*)CC2)cc1F]

In some embodiments, L is

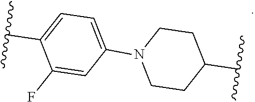 [SMILES: *c1ccc(N2CCC(*)CC2)cc1F]

In some embodiments, L is

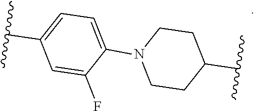 [SMILES: *c1ccc(N2CCC(*)CC2)c(F)c1]

In some embodiments, L is

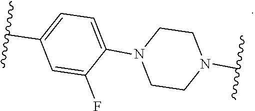 [SMILES: *c1ccc(N2CCN(*)CC2)c(F)c1]

In some embodiments, L is

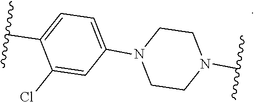 [SMILES: *c1ccc(N2CCN(*)CC2)cc1Cl]

In some embodiments, L is

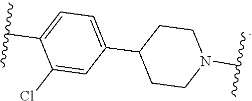 [SMILES: *c1ccc(C2CCN(*)CC2)cc1Cl]

In some embodiments, L is

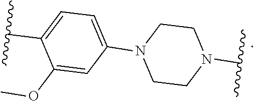 [SMILES: *c1ccc(N2CCN(*)CC2)cc1OC]

In some embodiments, L is

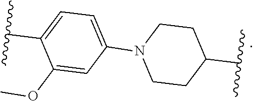 [SMILES: *c1ccc(N2CCC(*)CC2)cc1OC]

In some embodiments, L is

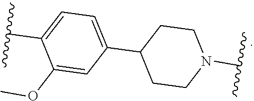 [SMILES: *c1ccc(C2CCN(*)CC2)cc1OC]

In some embodiments, L is

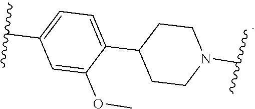 [SMILES: *c1ccc(C2CCN(*)CC2)c(OC)c1]

In some embodiments, L is

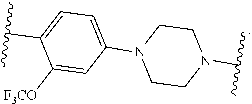 [SMILES: *c1ccc(N2CCN(*)CC2)cc1OC(F)(F)F]

In some embodiments, L is

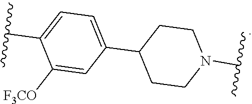 [SMILES: *c1ccc(C2CCN(*)CC2)cc1OC(F)(F)F]

In some embodiments, L is

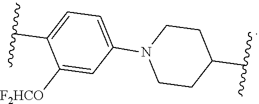 [SMILES: *c1ccc(N2CCC(*)CC2)cc1OC(F)F]

In some embodiments, L is

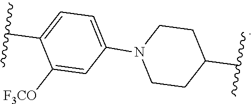 [SMILES: *c1ccc(N2CCC(*)CC2)cc1OC(F)(F)F]

In some embodiments, L is

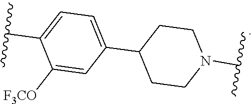 [SMILES: *c1ccc(C2CCN(*)CC2)cc1OC(F)(F)F]

In some embodiments, L is

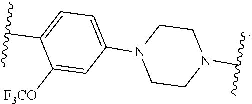 [SMILES: *c1ccc(N2CCN(*)CC2)cc1OC(F)(F)F]

In some embodiments, L is

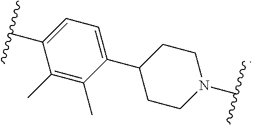 [SMILES: *c1ccc(C2CCN(*)CC2)c(C)c1C]

In some embodiments, L is

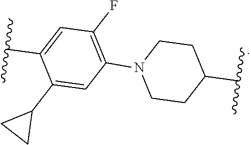 [SMILES: *c1cc(F)c(N2CCC(*)CC2)cc1C1CC1]

In some embodiments, L is

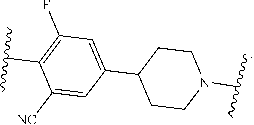 [SMILES: *c1c(F)cc(C2CCN(*)CC2)cc1C#N]

In some embodiments, L is

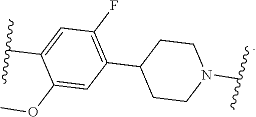 [SMILES: *c1cc(F)c(C2CCN(*)CC2)cc1OC]

In some embodiments, L is

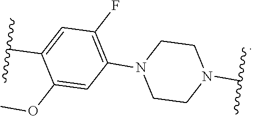 [SMILES: *c1cc(F)c(N2CCN(*)CC2)cc1OC]

In some embodiments, L is

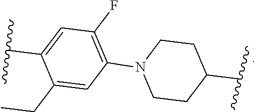 [SMILES: *c1cc(F)c(N2CCC(*)CC2)cc1CC]

In some embodiments, L is

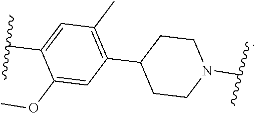 [SMILES: *c1cc(C)c(C2CCN(*)CC2)cc1OC]

In some embodiments, L is

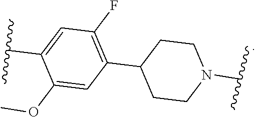 [SMILES: *c1cc(F)c(C2CCN(*)CC2)cc1OC]

In some embodiments, L is

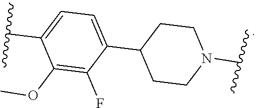 [SMILES: *c1ccc(C2CCN(*)CC2)c(F)c1OC]

In some embodiments, L is

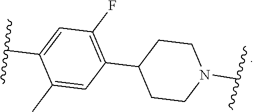 [SMILES: *c1cc(F)c(C2CCN(*)CC2)cc1C]

In some embodiments, L is

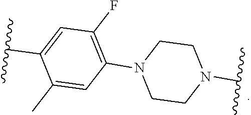 [SMILES: *c1cc(F)c(N2CCN(*)CC2)cc1C]

In some embodiments, L is

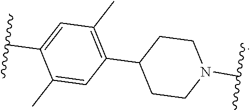 [SMILES: *c1cc(C)c(C2CCN(*)CC2)cc1C]

In some embodiments, L is

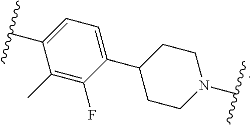 [SMILES: *c1ccc(C2CCN(*)CC2)c(F)c1C]

In some embodiments, L is

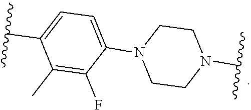 [SMILES: *c1ccc(N2CCN(*)CC2)c(F)c1C]

In some embodiments, L is

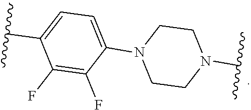 [SMILES: *c1ccc(N2CCN(*)CC2)c(F)c1F]

In some embodiments, L is

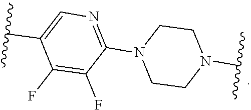 [SMILES: *c1cnc(N2CCN(*)CC2)c(F)c1F]

In some embodiments, L is

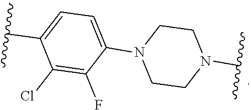 [SMILES: *c1ccc(N2CCN(*)CC2)c(F)c1Cl]

In some embodiments, L is

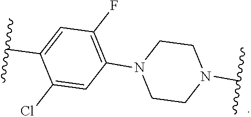 [SMILES: *c1cc(F)c(N2CCN(*)CC2)cc1Cl]

In some embodiments, L is

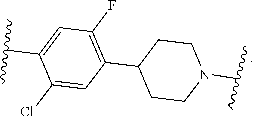 [SMILES: *c1cc(F)c(C2CCN(*)CC2)cc1Cl]

In some embodiments, L is

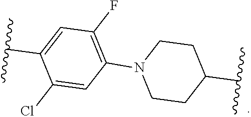 [SMILES: *c1cc(F)c(N2CCC(*)CC2)cc1Cl]

In some embodiments, L is

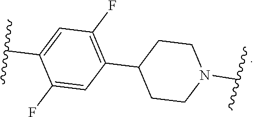 [SMILES: *c1cc(F)c(C2CCN(*)CC2)cc1F]

In some embodiments, L is

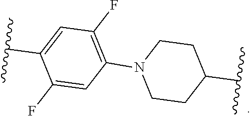 [SMILES: *c1cc(F)c(N2CCC(*)CC2)cc1F]

In some embodiments, L is

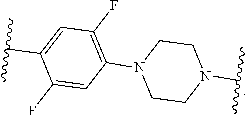 [SMILES: *c1cc(F)c(N2CCN(*)CC2)cc1F]

In some embodiments, L is

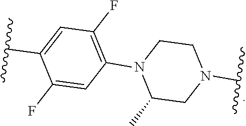 [SMILES: *c1cc(F)c(N2CCN(*)C[C@@H]2C)cc1F]

In some embodiments, L is

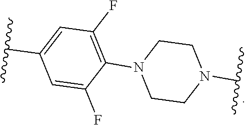 [SMILES: *c1cc(F)c(N2CCN(*)CC2)c(F)c1]

In some embodiments, L is

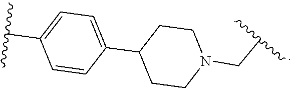 [SMILES: *CN1CCC(c2ccc(*)cc2)CC1]

In some embodiments, L is

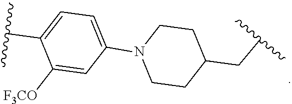 [SMILES: *CC1CCN(c2ccc(*)c(OC(F)(F)F)c2)CC1]

In some embodiments, L is

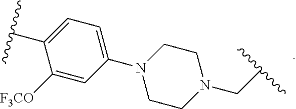 [SMILES: *CN1CCN(c2ccc(*)c(OC(F)(F)F)c2)CC1]

In some embodiments, L is

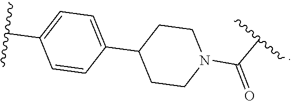 [SMILES: *C(=O)N1CCC(c2ccc(*)cc2)CC1]

In some embodiments, L is

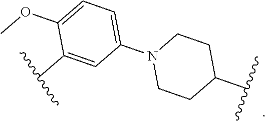 [SMILES: *c1cc(N2CCC(*)CC2)ccc1OC]

In some embodiments, L is

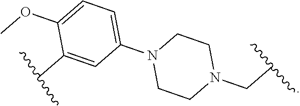 [SMILES: *CN1CCN(c2ccc(OC)c(*)c2)CC1]

In some embodiments, L is

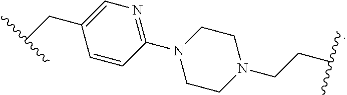 [SMILES: *CCN1CCN(c2ccc(C*)cn2)CC1]

In some embodiments, L is

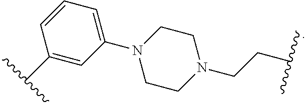 [SMILES: *CCN1CCN(c2cccc(*)c2)CC1]

In some embodiments, L is

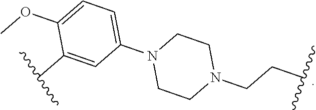 [SMILES: *CCN1CCN(c2ccc(OC)c(*)c2)CC1]

In some embodiments, L is

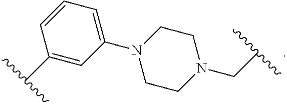 [SMILES: *CN1CCN(c2cccc(*)c2)CC1]

In some embodiments, L is

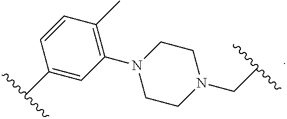 [SMILES: *CN1CCN(c2cc(*)ccc2C)CC1]

In some embodiments, L is

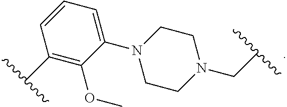 [SMILES: *CN1CCN(c2cccc(*)c2OC)CC1]

In some embodiments, L is

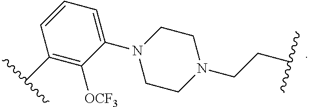 [SMILES: *CCN1CCN(c2cccc(*)c2OC(F)(F)F)CC1]

In some embodiments, L is

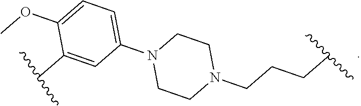 [SMILES: *CCCN1CCN(c2ccc(OC)c(*)c2)CC1]

In some embodiments, L is

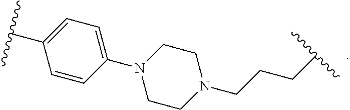 [SMILES: *CCCN1CCN(c2ccc(*)cc2)CC1]

In some embodiments, L is

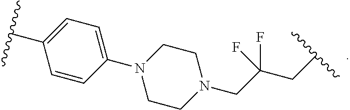 [SMILES: *CC(F)(F)CN1CCN(c2ccc(*)cc2)CC1]

In some embodiments, L is

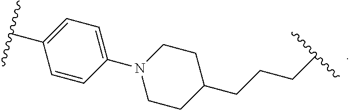 [SMILES: *CCCC1CCN(c2ccc(*)cc2)CC1]

In some embodiments, L is

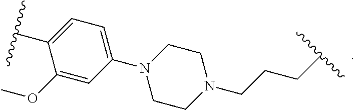 [SMILES: *CCCN1CCN(c2ccc(*)c(OC)c2)CC1]

In some embodiments, L is

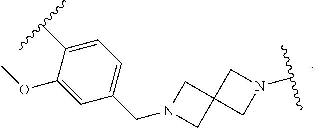 [SMILES: *c1ccc(CN2CC3(CN(*)C3)C2)cc1OC]

In some embodiments, L is

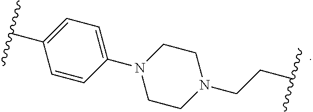 [SMILES: *CCN1CCN(c2ccc(*)cc2)CC1]

In some embodiments, L is

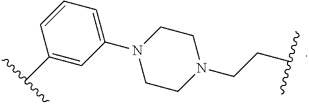 [SMILES: *CCN1CCN(c2cccc(*)c2)CC1]

In some embodiments, L is

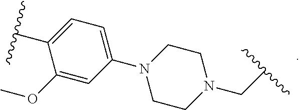 [SMILES: *CN1CCN(c2ccc(*)c(OC)c2)CC1]

In some embodiments, L is

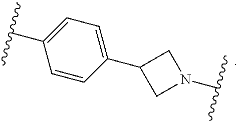 [SMILES: *c1ccc(C2CN(*)C2)cc1]

In some embodiments, L is

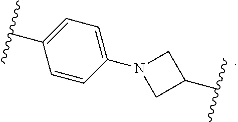 [SMILES: *c1ccc(N2CC(*)C2)cc1]

In some embodiments, L is

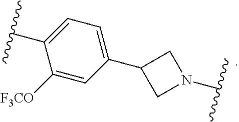 [SMILES: *c1ccc(C2CN(*)C2)cc1OC(F)(F)F]

In some embodiments, L is

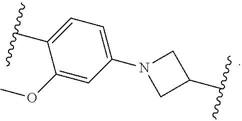 [SMILES: *c1ccc(N2CC(*)C2)cc1OC]

In some embodiments, L is

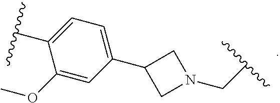 [SMILES: *CN1CC(c2ccc(*)c(OC)c2)C1]

In some embodiments, L is

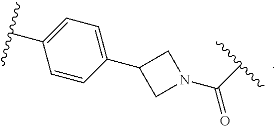 [SMILES: *C(=O)N1CC(c2ccc(*)cc2)C1]

In some embodiments, L is

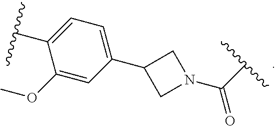 [SMILES: *C(=O)N1CC(c2ccc(*)c(OC)c2)C1]

In some embodiments, L is

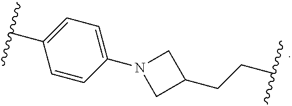 [SMILES: *CCC1CN(c2ccc(*)cc2)C1]

In some embodiments, L is

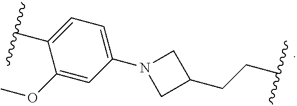 [SMILES: *CCC1CN(c2ccc(*)c(OC)c2)C1]

In some embodiments, L is

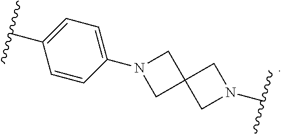 [SMILES: *c1ccc(N2CC3(CN(*)C3)C2)cc1]

In some embodiments, L is

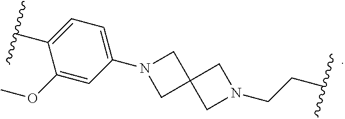 [SMILES: *CCN1CC2(C1)CN(c1ccc(*)c(OC)c1)C2]

In some embodiments, L is

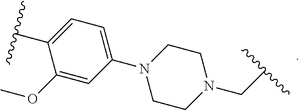 [SMILES: *CN1CCN(c2ccc(*)c(OC)c2)CC1]

In some embodiments, L is

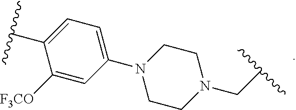 [SMILES: *CN1CCN(c2ccc(*)c(OC(F)(F)F)c2)CC1]

In some embodiments, L is

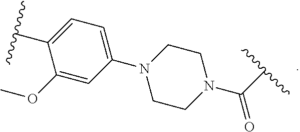 [SMILES: *C(=O)N1CCN(c2ccc(*)c(OC)c2)CC1]

In some embodiments, L is

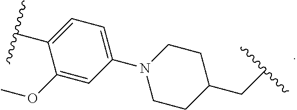 [SMILES: *CC1CCN(c2ccc(*)c(OC)c2)CC1]

In some embodiments, L is

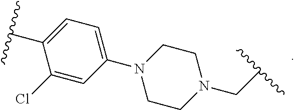 [SMILES: *CN1CCN(c2ccc(*)c(Cl)c2)CC1]

In some embodiments, L is

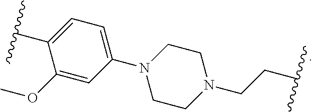 [SMILES: *CCN1CCN(c2ccc(*)c(OC)c2)CC1]

In some embodiments, L is

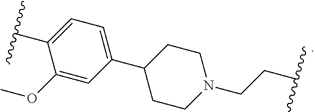 [SMILES: *CCN1CCC(c2ccc(*)c(OC)c2)CC1]

In some embodiments, L is

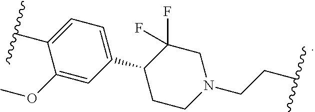 [SMILES: *CCN1CC[C@H](c2ccc(*)c(OC)c2)C(F)(F)C1]

In some embodiments, L is

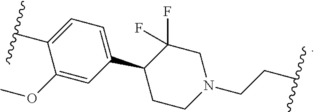 [SMILES: *CCN1CC[C@@H](c2ccc(*)c(OC)c2)C(F)(F)C1]

In some embodiments, L is

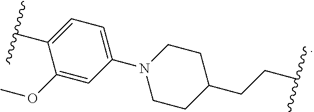 [SMILES: *CCC1CCN(c2ccc(*)c(OC)c2)CC1]

In some embodiments, L is

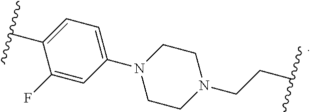 [SMILES: *CCN1CCN(c2ccc(*)c(F)c2)CC1]

In some embodiments, L is

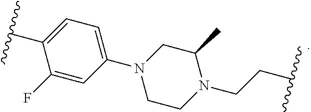 [SMILES: *CCN1CCN(c2ccc(*)c(F)c2)C[C@H]1C]

In some embodiments, L is

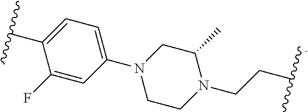 [SMILES: *CCN1CCN(c2ccc(*)c(F)c2)C[C@@H]1C]

In some embodiments, L is

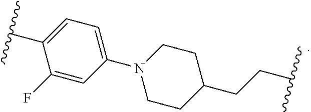 [SMILES: *CCC1CCN(c2ccc(*)c(F)c2)CC1]

In some embodiments, L is

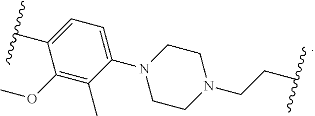 [SMILES: *CCN1CCN(c2ccc(*)c(OC)c2C)CC1]

In some embodiments, L is

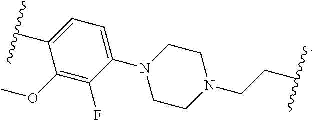 [SMILES: *CCN1CCN(c2ccc(*)c(OC)c2F)CC1]

In some embodiments, L is

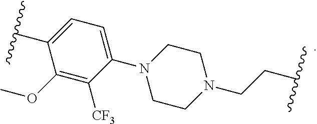 [SMILES: *CCN1CCN(c2ccc(*)c(OC)c2C(F)(F)F)CC1]

In some embodiments, L is

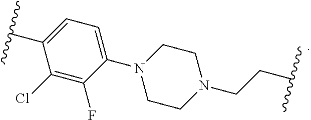 [SMILES: *CCN1CCN(c2ccc(*)c(Cl)c2F)CC1]

In some embodiments, L is

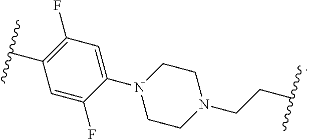 [SMILES: *CCN1CCN(c2cc(F)c(*)cc2F)CC1]

In some embodiments, L is

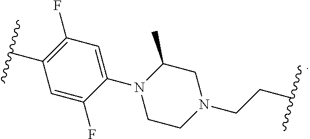 [SMILES: *CCN1CCN(c2cc(F)c(*)cc2F)[C@@H](C)C1]

In some embodiments, L is

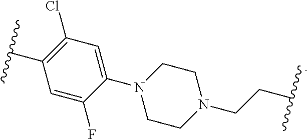 [SMILES: *CCN1CCN(c2cc(Cl)c(*)cc2F)CC1]

In some embodiments, L is

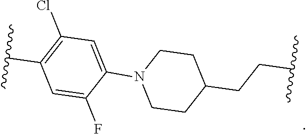 [SMILES: *CCC1CCN(c2cc(Cl)c(*)cc2F)CC1]

In some embodiments, L is

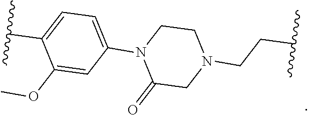 [SMILES: *CCN1CCN(c2ccc(*)c(OC)c2)C(=O)C1]

In some embodiments, L is

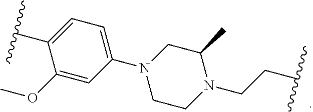 [SMILES: *CCN1CCN(c2ccc(*)c(OC)c2)C[C@H]1C]

In some embodiments, L is

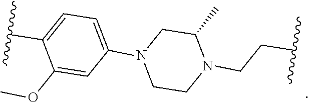 [SMILES: *CCN1CCN(c2ccc(*)c(OC)c2)C[C@@H]1C]

In some embodiments, L is

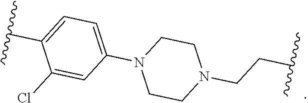 [SMILES: *CCN1CCN(c2ccc(*)c(Cl)c2)CC1]

In some embodiments, L is

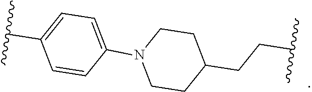 [SMILES: *CCC1CCN(c2ccc(*)cc2)CC1]

In some embodiments, L is

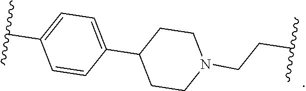 [SMILES: *CCN1CCC(c2ccc(*)cc2)CC1]

In some embodiments, L is

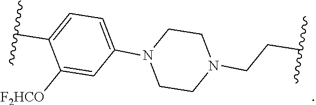 [SMILES: *CCN1CCN(c2ccc(*)c(OC(F)F)c2)CC1]

In some embodiments, L is

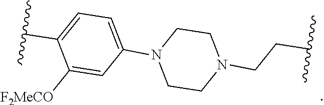

In some embodiments, L is

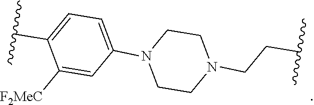

In some embodiments, L is

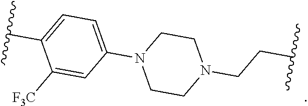 [SMILES: *CCN1CCN(c2ccc(*)c(C(F)(F)F)c2)CC1]

In some embodiments, L is

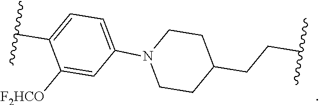 [SMILES: *CCC1CCN(c2ccc(*)c(OC(F)F)c2)CC1]

In some embodiments, L is

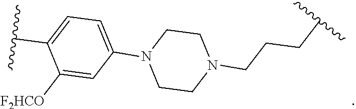 [SMILES: *CCCN1CCN(c2ccc(*)c(OC(F)F)c2)CC1]

In some embodiments, L is

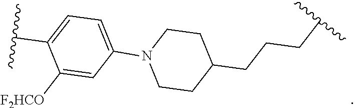 [SMILES: *CCCC1CCN(c2ccc(*)c(OC(F)F)c2)CC1]

In some embodiments, L is

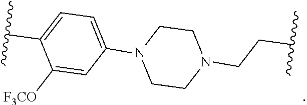 [SMILES: *CCN1CCN(c2ccc(*)c(OC(F)(F)F)c2)CC1]

In some embodiments, L is

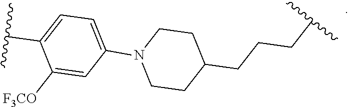 [SMILES: *CCCC1CCN(c2ccc(*)c(OC(F)(F)F)c2)CC1]

In some embodiments, L is

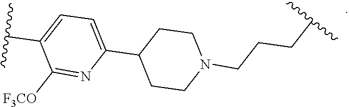 [SMILES: *CCCN1CCC(c2ccc(*)c(OC(F)(F)F)n2)CC1]

In some embodiments, L is

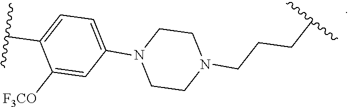 [SMILES: *CCCN1CCN(c2ccc(*)c(OC(F)(F)F)c2)CC1]

In some embodiments, L is

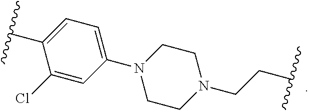 [SMILES: *CCN1CCN(c2ccc(*)c(Cl)c2)CC1]

In some embodiments, L is

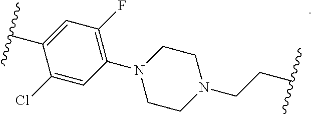 [SMILES: *CCN1CCN(c2cc(Cl)c(*)cc2F)CC1]

In some embodiments, L is

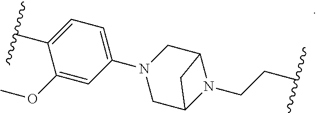 [SMILES: *CCN1C2CC1CN(c1ccc(*)c(OC)c1)C2]

In some embodiments, L is

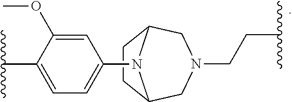 [SMILES: *CCN1CC2CCC(C1)N2c1ccc(*)c(OC)c1]

In some embodiments, L is

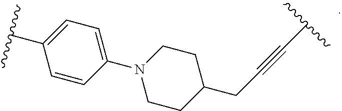 [SMILES: *C#CCC1CCN(c2ccc(*)cc2)CC1]

In some embodiments, L is

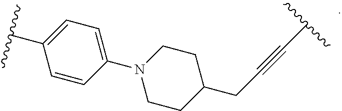 [SMILES: *C#CCC1CCN(c2ccc(*)cc2)CC1]

In some embodiments, L is

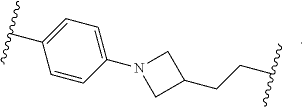 [SMILES: *CCC1CN(c2ccc(*)cc2)C1]

In some embodiments, L is

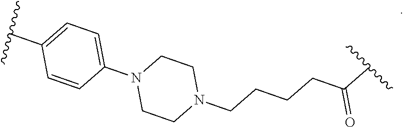 [SMILES: *C(=O)CCCCN1CCN(c2ccc(*)cc2)CC1]

In some embodiments, L is

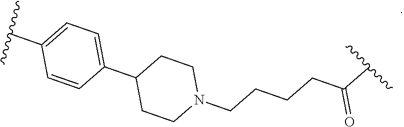 [SMILES: *C(=O)CCCCN1CCC(c2ccc(*)cc2)CC1]

In some embodiments, L is

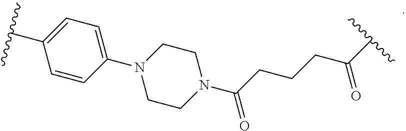 [SMILES: *C(=O)CCCC(=O)N1CCN(c2ccc(*)cc2)CC1]

In some embodiments, L is

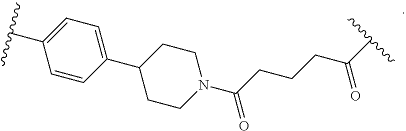 [SMILES: *C(=O)CCCC(=O)N1CCC(c2ccc(*)cc2)CC1]

In some embodiments, L is

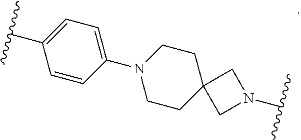 [SMILES: *c1ccc(N2CCC3(CC2)CN(*)C3)cc1]

In some embodiments, L is

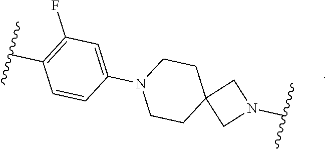 [SMILES: *c1ccc(N2CCC3(CC2)CN(*)C3)cc1F]

In some embodiments, L is

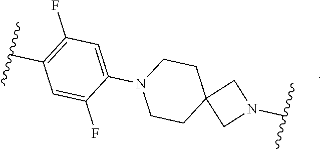 [SMILES: *c1cc(F)c(N2CCC3(CC2)CN(*)C3)cc1F]

In some embodiments, L is

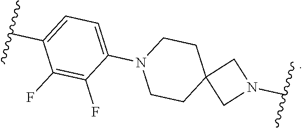 [SMILES: *c1ccc(N2CCC3(CC2)CN(*)C3)c(F)c1F]

In some embodiments, L is

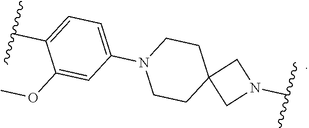 [SMILES: *c1ccc(N2CCC3(CC2)CN(*)C3)cc1OC]

In some embodiments, L is

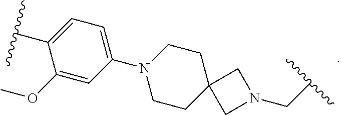 [SMILES: *CN1CC2(CCN(c3ccc(*)c(OC)c3)CC2)C1]

In some embodiments, L is

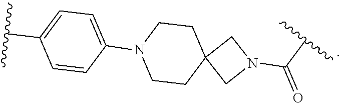 [SMILES: *C(=O)N1CC2(CCN(c3ccc(*)cc3)CC2)C1]

In some embodiments, L is

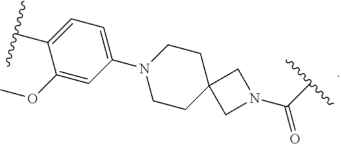 [SMILES: *C(=O)N1CC2(CCN(c3ccc(*)c(OC)c3)CC2)C1]

In some embodiments, L is

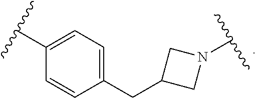 [SMILES: *c1ccc(CC2CN(*)C2)cc1]

In some embodiments, L is

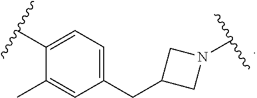 [SMILES: *c1ccc(CC2CN(*)C2)cc1C]

In some embodiments, L is

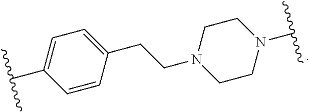 [SMILES: *c1ccc(CCN2CCN(*)CC2)cc1]

In some embodiments, L is

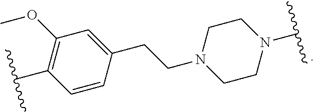 [SMILES: *c1ccc(CCN2CCN(*)CC2)cc1OC]

In some embodiments, L is

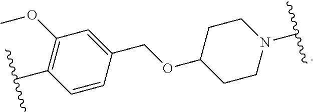 [SMILES: *c1ccc(COC2CCN(*)CC2)cc1OC]

In some embodiments, L is

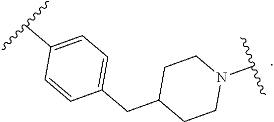 [SMILES: *c1ccc(CC2CCN(*)CC2)cc1]

In some embodiments, L is

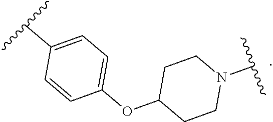 [SMILES: *c1ccc(OC2CCN(*)CC2)cc1]

In some embodiments, L is

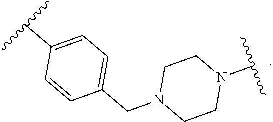 [SMILES: *c1ccc(CN2CCN(*)CC2)cc1]

In some embodiments, L is

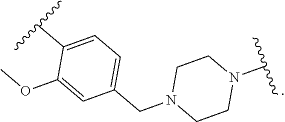 [SMILES: *c1ccc(CN2CCN(*)CC2)cc1OC]

In some embodiments, L is

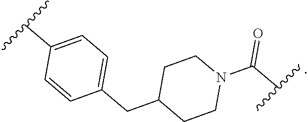 [SMILES: *C(=O)N1CCC(Cc2ccc(*)cc2)CC1]

In some embodiments, L is

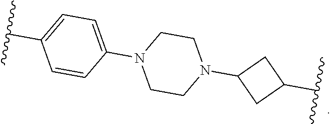 [SMILES: *c1ccc(N2CCN(C3CC(*)C3)CC2)cc1]

In some embodiments, L is

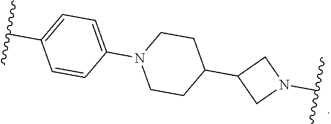 [SMILES: *c1ccc(N2CCC(C3CN(*)C3)CC2)cc1]

In some embodiments, L is

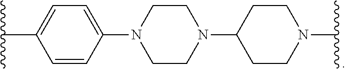 [SMILES: *c1ccc(N2CCN(C3CCN(*)CC3)CC2)cc1]

In some embodiments, L is

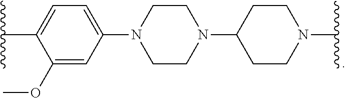 [SMILES: *c1ccc(N2CCN(C3CCN(*)CC3)CC2)cc1OC]

In some embodiments, L is

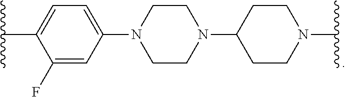 [SMILES: *c1ccc(N2CCN(C3CCN(*)CC3)CC2)cc1F]

In some embodiments, L is

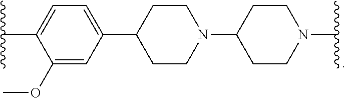 [SMILES: *c1ccc(C2CCN(C3CCN(*)CC3)CC2)cc1OC]

In some embodiments, L is

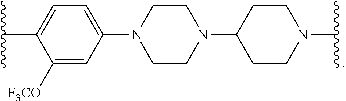 [SMILES: *c1ccc(N2CCN(C3CCN(*)CC3)CC2)cc1OC(F)(F)F]

In some embodiments, L is

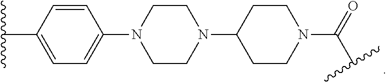 [SMILES: *C(=O)N1CCC(N2CCN(c3ccc(*)cc3)CC2)CC1]

In some embodiments, L is

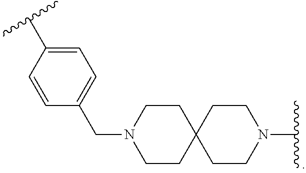 [SMILES: *c1ccc(CN2CCC3(CCN(*)CC3)CC2)cc1]

In some embodiments, L is

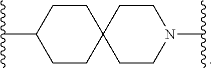 [SMILES: *C1CCC2(CC1)CCN(*)CC2]

In some embodiments, L is

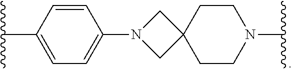 [SMILES: *c1ccc(N2CC3(CCN(*)CC3)C2)cc1]

In some embodiments, L is

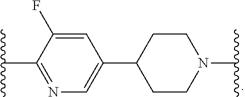 [SMILES: *c1ncc(C2CCN(*)CC2)cc1F]

In some embodiments, L is

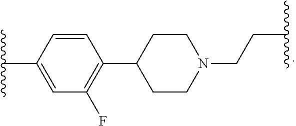 [SMILES: *CCN1CCC(c2ccc(*)cc2F)CC1]

In some embodiments, L is

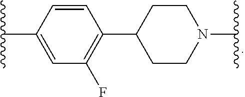 [SMILES: *c1ccc(C2CCN(*)CC2)c(F)c1]

In some embodiments, L is

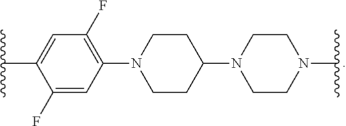 [SMILES: *c1cc(F)c(N2CCC(N3CCN(*)CC3)CC2)cc1F]

In some embodiments, L is

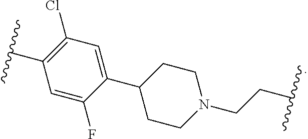 [SMILES: *CCN1CCC(c2cc(Cl)c(*)cc2F)CC1]

In some embodiments, L is

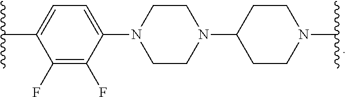 [SMILES: *c1ccc(N2CCN(C3CCN(*)CC3)CC2)c(F)c1F]

In some embodiments, L is

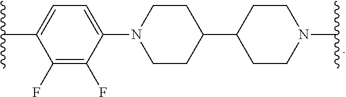 [SMILES: *c1ccc(N2CCC(C3CCN(*)CC3)CC2)c(F)c1F]

In some embodiments, L is

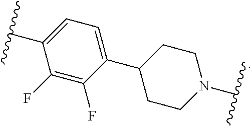 [SMILES: *c1ccc(C2CCN(*)CC2)c(F)c1F]

In some embodiments, L is

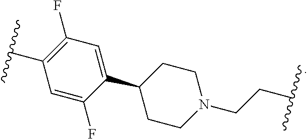

In some embodiments, L is

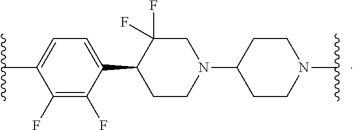 [SMILES: *c1ccc([C@@H]2CCN(C3CCN(*)CC3)CC2(F)F)c(F)c1F]

In some embodiments, L is

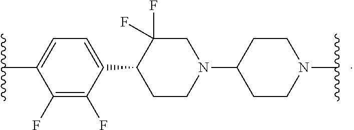 [SMILES: *c1ccc([C@H]2CCN(C3CCN(*)CC3)CC2(F)F)c(F)c1F]

In some embodiments, L is

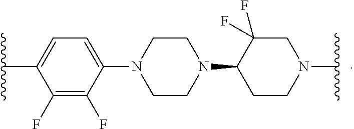 [SMILES: *c1ccc(N2CCN([C@@H]3CCN(*)CC3(F)F)CC2)c(F)c1F]

In some embodiments, L is

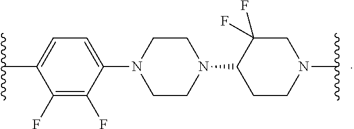

In some embodiments, L is

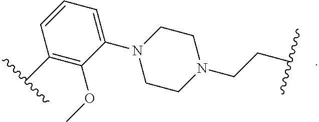 [SMILES: *CCN1CCN(c2cccc(*)c2OC)CC1]

In some embodiments, L is

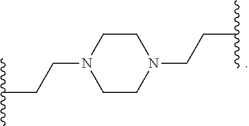 [SMILES: *CCN1CCN(CC*)CC1]

In some embodiments, L is

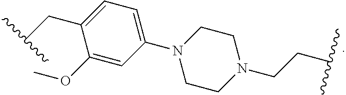 [SMILES: *CCN1CCN(c2ccc(C*)c(OC)c2)CC1]

In some embodiments, L is

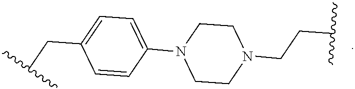 [SMILES: *CCN1CCN(c2ccc(C*)cc2)CC1]

In some embodiments, L is

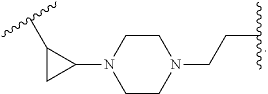 [SMILES: *CCN1CCN(C2CC2*)CC1]

In some embodiments, L is

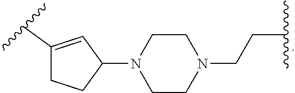 [SMILES: *CCN1CCN(C2C=C(*)CC2)CC1]

In some embodiments, L is

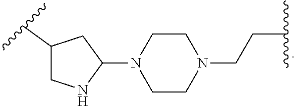 [SMILES: *CCN1CCN(C2CC(*)CN2)CC1]

In some embodiments, L is

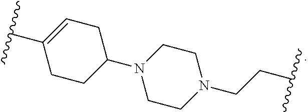 [SMILES: *CCN1CCN(C2CC=C(*)CC2)CC1]

In some embodiments, L is

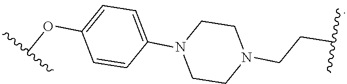 [SMILES: *CCN1CCN(c2ccc(O*)cc2)CC1]

In some embodiments, L is

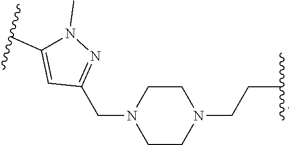 [SMILES: *CCN1CCN(Cc2cc(*)n(C)n2)CC1]

In some embodiments, L is

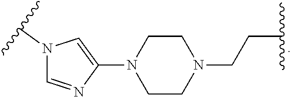 [SMILES: *CCN1CCN(c2cn(*)cn2)CC1]

In some embodiments, L is

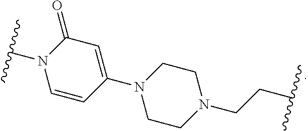 [SMILES: *CCN1CCN(c2ccn(*)c(=O)c2)CC1]

In some embodiments, L is

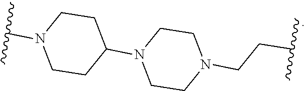 [SMILES: *CCN1CCN(C2CCN(*)CC2)CC1]

In some embodiments, L is

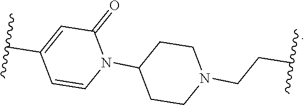 [SMILES: *CCN1CCC(n2ccc(*)cc2=O)CC1]

In some embodiments, L is

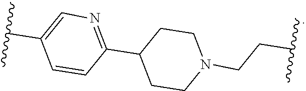 [SMILES: *CCN1CCC(c2ccc(*)cn2)CC1]

In some embodiments, L is

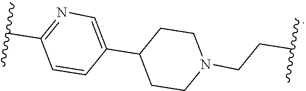 [SMILES: *CCN1CCC(c2ccc(*)nc2)CC1]

In some embodiments, L is

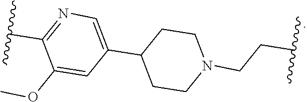 [SMILES: *CCN1CCC(c2cnc(*)c(OC)c2)CC1]

In some embodiments, L is

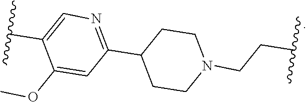 [SMILES: *CCN1CCC(c2cc(OC)c(*)cn2)CC1]

In some embodiments, L is

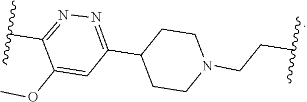 [SMILES: *CCN1CCC(c2cc(OC)c(*)nn2)CC1]

In some embodiments, L is

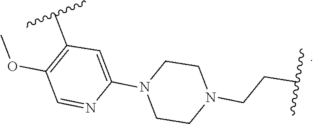 [SMILES: *CCN1CCN(c2cc(*)c(OC)cn2)CC1]

In some embodiments, L is

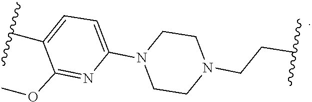 [SMILES: *CCN1CCN(c2ccc(*)c(OC)n2)CC1]

In some embodiments, L is

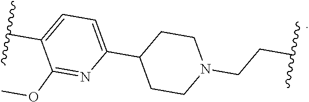 [SMILES: *CCN1CCC(c2ccc(*)c(OC)n2)CC1]

In some embodiments, L is

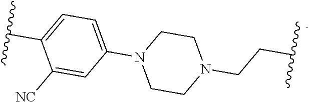 [SMILES: *CCN1CCN(c2ccc(*)c(C#N)c2)CC1]

In some embodiments, L is

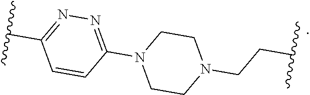 [SMILES: *CCN1CCN(c2ccc(*)nn2)CC1]

In some embodiments, L is

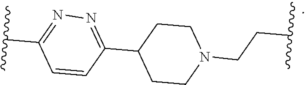 [SMILES: *CCN1CCC(c2ccc(*)nn2)CC1]

In some embodiments, L is

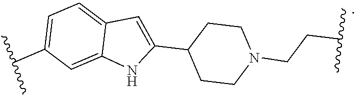 [SMILES: *CCN1CCC(c2cc3ccc(*)cc3[nH]2)CC1]

In some embodiments, L is

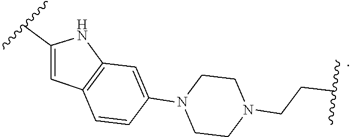 [SMILES: *CCN1CCN(c2ccc3cc(*)[nH]c3c2)CC1]

In some embodiments, L is

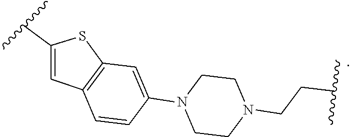 [SMILES: *CCN1CCN(c2ccc3cc(*)sc3c2)CC1]

In some embodiments, L is

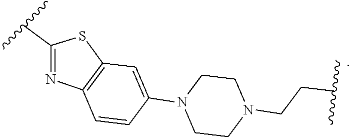 [SMILES: *CCN1CCN(c2ccc3nc(*)sc3c2)CC1]

In some embodiments, L is

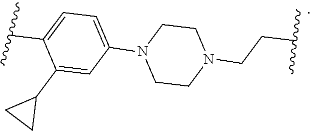 [SMILES: *CCN1CCN(c2ccc(*)c(C3CC3)c2)CC1]

In some embodiments, L is

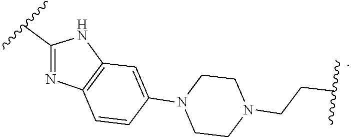 [SMILES: *CCN1CCN(c2ccc3nc(*)[nH]c3c2)CC1]

In some embodiments, L is

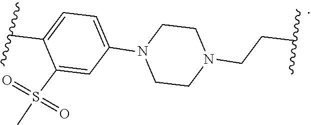 [SMILES: *CCN1CCN(c2ccc(*)c(S(C)(=O)=O)c2)CC1]

In some embodiments, L is

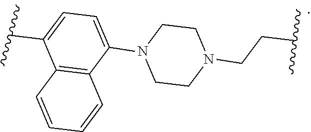 [SMILES: *CCN1CCN(c2ccc(*)c3ccccc23)CC1]

In some embodiments, L is

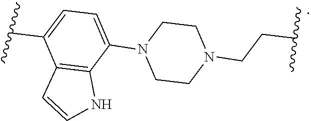 [SMILES: *CCN1CCN(c2ccc(*)c3cc[nH]c23)CC1]

In some embodiments, L is

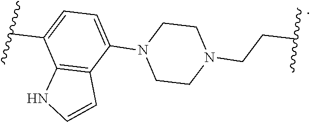 [SMILES: *CCN1CCN(c2ccc(*)c3[nH]ccc23)CC1]

In some embodiments, L is

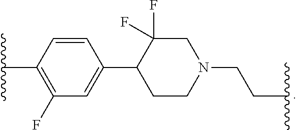 [SMILES: *CCN1CCC(c2ccc(*)c(F)c2)C(F)(F)C1]

In some embodiments, L is

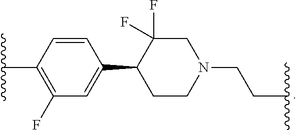 [SMILES: *CCN1CC[C@@H](c2ccc(*)c(F)c2)C(F)(F)C1]

In some embodiments, L is

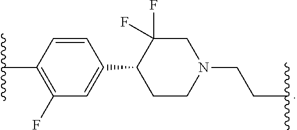 [SMILES: *CCN1CC[C@H](c2ccc(*)c(F)c2)C(F)(F)C1]

In some embodiments, L is

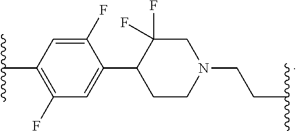 [SMILES: *CCN1CCC(c2cc(F)c(*)cc2F)C(F)(F)C1]

In some embodiments, L is

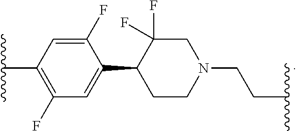 [SMILES: *CCN1CC[C@@H](c2cc(F)c(*)cc2F)C(F)(F)C1]

In some embodiments, L is

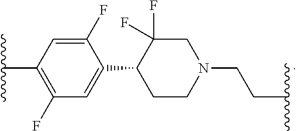 [SMILES: *CCN1CC[C@H](c2cc(F)c(*)cc2F)C(F)(F)C1]

In some embodiments, L is

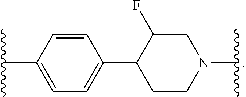 [SMILES: *c1ccc(C2CCN(*)CC2F)cc1]

In some embodiments, L is

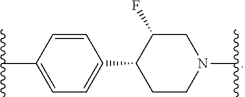 [SMILES: *c1ccc([C@H]2CCN(*)C[C@H]2F)cc1]

In some embodiments, L is

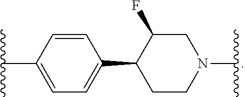 [SMILES: *c1ccc([C@@H]2CCN(*)C[C@@H]2F)cc1]

In some embodiments, L is

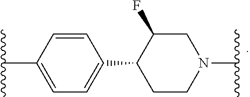 [SMILES: *c1ccc([C@H]2CCN(*)C[C@@H]2F)cc1]

In some embodiments, L is

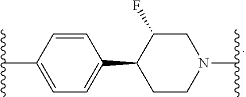 [SMILES: *c1ccc([C@@H]2CCN(*)C[C@H]2F)cc1]

In some embodiments, L is

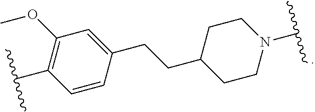 [SMILES: *c1ccc(CCC2CCN(*)CC2)cc1OC]

In some embodiments, L is

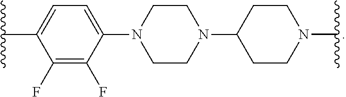 [SMILES: *c1ccc(N2CCN(C3CCN(*)CC3)CC2)c(F)c1F]

In some embodiments, L is

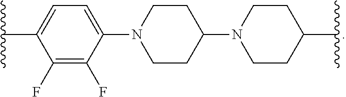 [SMILES: *c1ccc(N2CCC(N3CCC(*)CC3)CC2)c(F)c1F]

In some embodiments, L is selected from those depicted in Table 1B, below. In some embodiments, LBM is selected from those depicted in Table A, below. In some embodiments, L is selected from those depicted in Table B, below.

TABLE A
Exemplified E3 Ligase Binding Moiety (LBM)

| | |
|---|---|
| 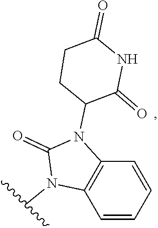 [SMILES: *n1c(=O)n(C2CCC(=O)NC2=O)c2ccccc21] | (e) |
| 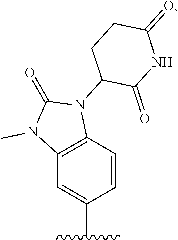 [SMILES: *c1ccc2c(c1)n(C)c(=O)n2C1CCC(=O)NC1=O] | (f) |
| 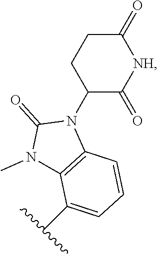 [SMILES: *c1cccc2c1n(C)c(=O)n2C1CCC(=O)NC1=O] | (g) |
| 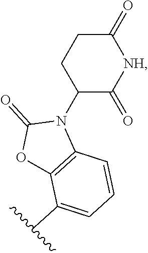 [SMILES: *c1cccc2c1oc(=O)n2C1CCC(=O)NC1=O] | (h) |
| 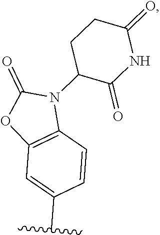 [SMILES: *c1ccc2c(c1)oc(=O)n2C1CCC(=O)NC1=O] | (i) |
| 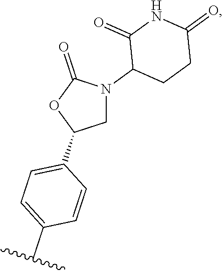 [SMILES: *c1ccc([C@H]2CN(C3CCC(=O)NC3=O)C(=O)O2)cc1] | (j) |
| 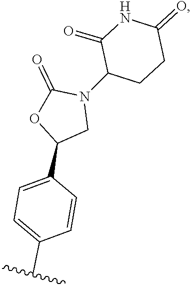 [SMILES: *c1ccc([C@@H]2CN(C3CCC(=O)NC3=O)C(=O)O2)cc1] | (k) |
| 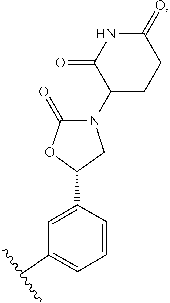 [SMILES: *c1cccc([C@H]2CN(C3CCC(=O)NC3=O)C(=O)O2)c1] | (l) |
| 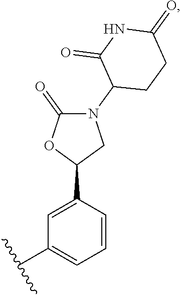 [SMILES: *c1cccc([C@@H]2CN(C3CCC(=O)NC3=O)C(=O)O2)c1] | (m) |
| 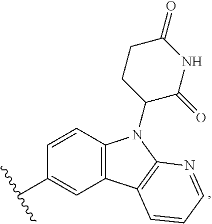 [SMILES: *c1ccc2c(c1)c1cccnc1n2C1CCC(=O)NC1=O] | (n) |
| 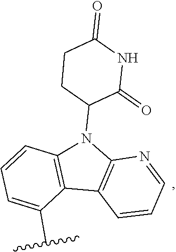 [SMILES: *c1cccc2c1c1cccnc1n2C1CCC(=O)NC1=O] | (o) |
| 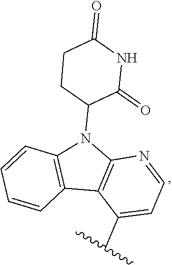 [SMILES: *c1ccnc2c1c1ccccc1n2C1CCC(=O)NC1=O] | (p) |
| 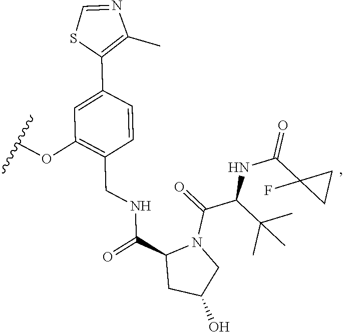 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (n) |
| 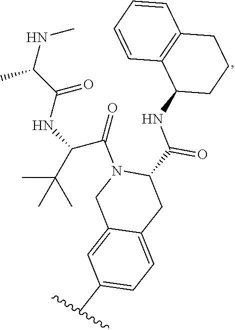 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (o) |
| 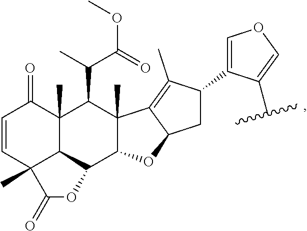 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (p) |
| 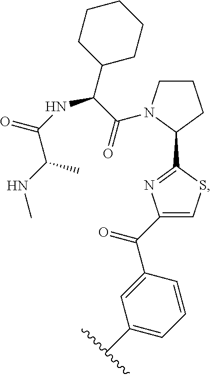 [SMILES: *c1cccc(C(=O)c2csc([C@@H]3CCCN3C(=O)[C@@H](NC(=O)[C@H](C)NC)C3CCCCC3)n2)c1] | (q) |
| 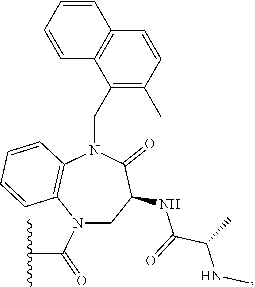 [SMILES: *C(=O)N1C[C@H](NC(=O)[C@H](C)NC)C(=O)N(Cc2c(C)ccc3ccccc23)c2ccccc21 ; ","] | (r) |
| 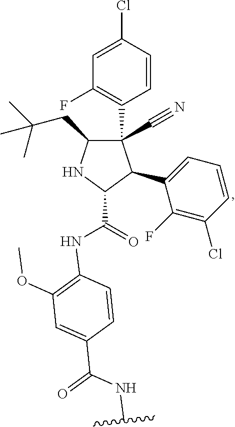 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; ","] | (s) |
| 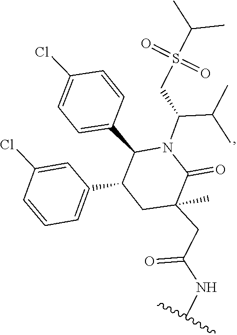 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (t) |
| 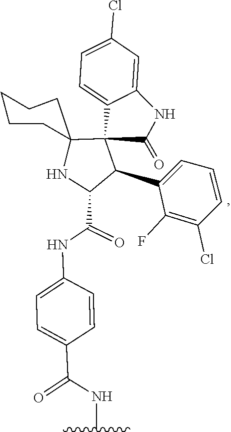 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (u) |
| 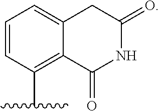 [SMILES: *c1cccc2c1C(=O)NC(=O)C2] | (v) |
| 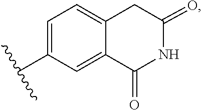 [SMILES: *c1ccc2c(c1)C(=O)NC(=O)C2] | (w) |
| 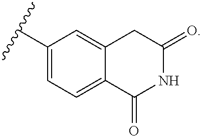 [SMILES: *c1ccc2c(c1)CC(=O)NC2=O] | (x) |
| 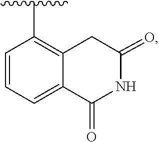 [SMILES: *c1cccc2c1CC(=O)NC2=O] | (y) |
| 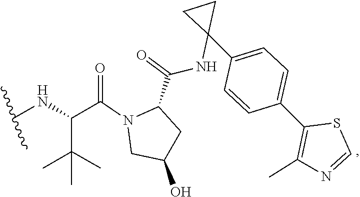 [SMILES: *N[C@H](C(=O)N1C[C@H](O)C[C@H]1C(=O)NC1(c2ccc(-c3scnc3C)cc2)CC1)C(C)(C)C] | (z) |
| 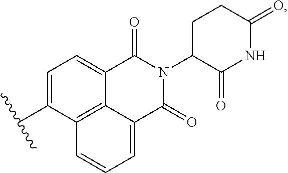 [SMILES: *c1ccc2c3c(cccc13)C(=O)N(C1CCC(=O)NC1=O)C2=O] | (bb) |
| 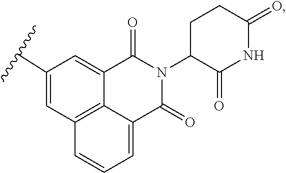 [SMILES: *c1cc2c3c(cccc3c1)C(=O)N(C1CCC(=O)NC1=O)C2=O] | (cc) |
| 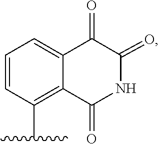 [SMILES: *c1cccc2c1C(=O)NC(=O)C2=O] | (dd) |
| 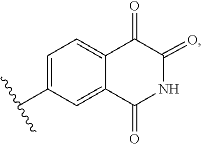 [SMILES: *c1ccc2c(c1)C(=O)NC(=O)C2=O] | (ee) |
| 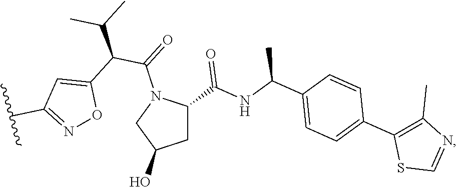 [SMILES: *c1cc([C@H](C(=O)N2C[C@H](O)C[C@H]2C(=O)N[C@@H](C)c2ccc(-c3scnc3C)cc2)C(C)C)on1] | (ff) |
| 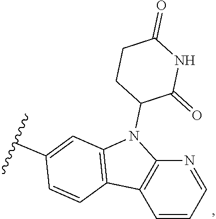 [SMILES: *c1ccc2c3cccnc3n(C3CCC(=O)NC3=O)c2c1] | (gg) |
| 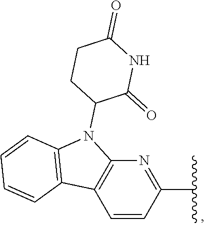 [SMILES: *c1ccc2c3ccccc3n(C3CCC(=O)NC3=O)c2n1] | (hh) |
| 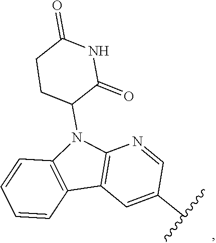 [SMILES: *c1cnc2c(c1)c1ccccc1n2C1CCC(=O)NC1=O] | (ii) |
| 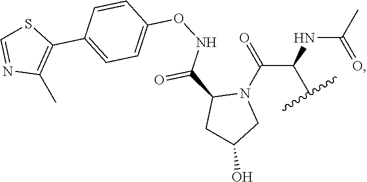 [SMILES: *[C@H](NC(C)=O)C(=O)N1C[C@H](O)C[C@H]1C(=O)NOc1ccc(-c2scnc2C)cc1] | (jj) |
| 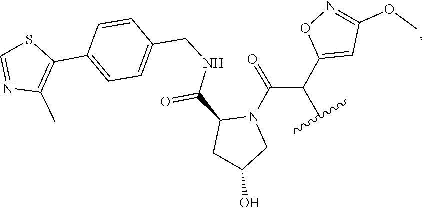 [SMILES: *C(C(=O)N1C[C@H](O)C[C@H]1C(=O)NCc1ccc(-c2scnc2C)cc1)c1cc(OC)no1] | (kk) |
| 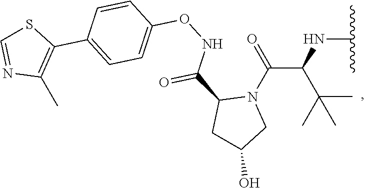 [SMILES: *N[C@H](C(=O)N1C[C@H](O)C[C@H]1C(=O)NOc1ccc(-c2scnc2C)cc1)C(C)(C)C] | (ll) |
| 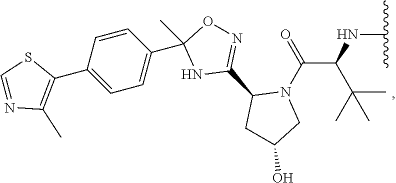 [SMILES: *N[C@H](C(=O)N1C[C@H](O)C[C@H]1C1=NOC(C)(c2ccc(-c3scnc3C)cc2)N1)C(C)(C)C] | (mm) |
| 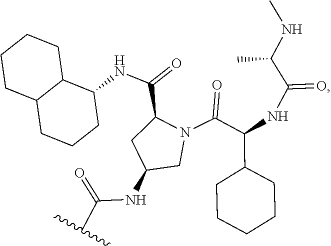 [SMILES: *C(=O)N[C@H]1C[C@@H](C(=O)N[C@@H]2CCCC3CCCCC32)N(C(=O)[C@@H](NC(=O)[C@H](C)NC)C2CCCCC2)C1] | (nn) |
| 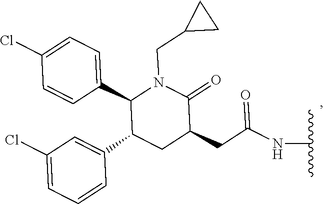 [SMILES: *NC(=O)C[C@H]1C[C@H](c2cccc(Cl)c2)[C@@H](c2ccc(Cl)cc2)N(CC2CC2)C1=O] | (oo) |
| 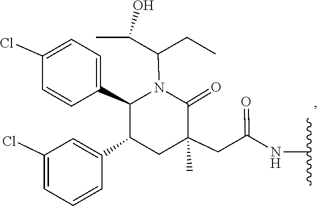 [SMILES: *NC(=O)C[C@@]1(C)C[C@H](c2cccc(Cl)c2)[C@@H](c2ccc(Cl)cc2)N(C(CC)[C@H](C)O)C1=O] | (pp) |
| 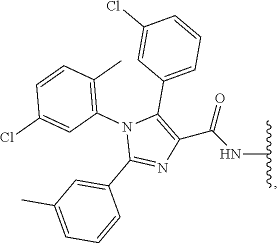 [SMILES: *NC(=O)c1nc(-c2cccc(C)c2)n(-c2cc(Cl)ccc2C)c1-c1cccc(Cl)c1] | (qq) |
| 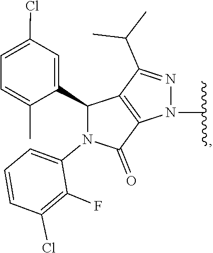 [SMILES: *n1nc(C(C)C)c2c1C(=O)N(c1cccc(Cl)c1F)[C@H]2c1cc(Cl)ccc1C] | (rr) |
| 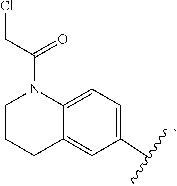 [SMILES: *c1ccc2c(c1)CCCN2C(=O)CCl ; ","] | (ss) |
| 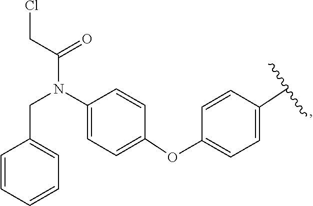 [SMILES: *c1ccc(Oc2ccc(N(Cc3ccccc3)C(=O)CCl)cc2)cc1] | (tt) |
| 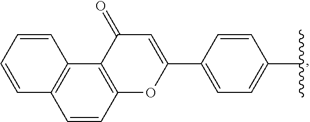 [SMILES: *c1ccc(-c2cc(=O)c3c(ccc4ccccc43)o2)cc1] | (uu) |
| 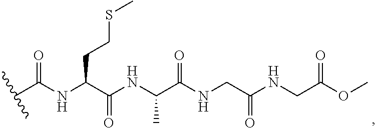 [SMILES: *C(=O)N[C@@H](CCSC)C(=O)N[C@@H](C)C(=O)NCC(=O)NCC(=O)OC] | (vv) |
| 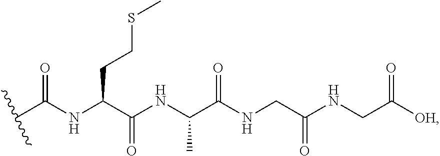 [SMILES: *C(=O)N[C@@H](CCSC)C(=O)N[C@@H](C)C(=O)NCC(=O)NCC(=O)O] | (ww) |
| 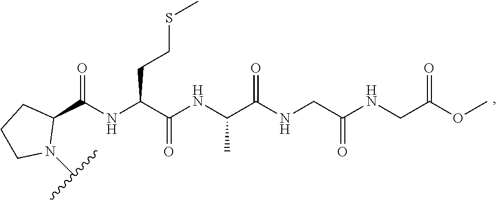 [SMILES: *N1CCC[C@H]1C(=O)N[C@@H](CCSC)C(=O)N[C@@H](C)C(=O)NCC(=O)NCC(=O)OC] | (xx) |
| 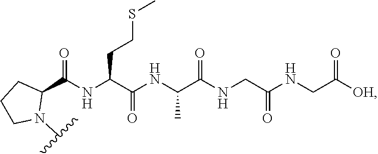 [SMILES: *N1CCC[C@H]1C(=O)N[C@@H](CCSC)C(=O)N[C@@H](C)C(=O)NCC(=O)NCC(=O)O] | (yy) |
| 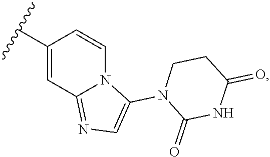 [SMILES: *c1ccn2c(N3CCC(=O)NC3=O)cnc2c1] | (zz) |
| 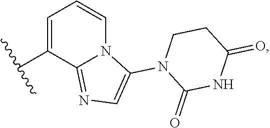 [SMILES: *c1cccn2c(N3CCC(=O)NC3=O)cnc12] | (aaa) |
| 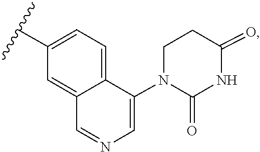 [SMILES: *c1ccc2c(N3CCC(=O)NC3=O)cncc2c1] | (bbb) |
| 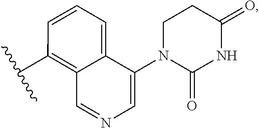 [SMILES: *c1cccc2c(N3CCC(=O)NC3=O)cncc12] | (ccc) |
| 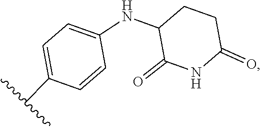 [SMILES: *c1ccc(NC2CCC(=O)NC2=O)cc1] | (ddd) |
| 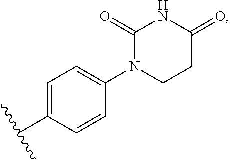 [SMILES: *c1ccc(N2CCC(=O)NC2=O)cc1] | (eee) |
| 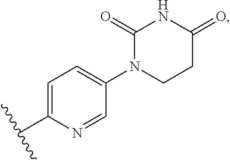 [SMILES: *c1ccc(N2CCC(=O)NC2=O)cn1] | (fff) |
| 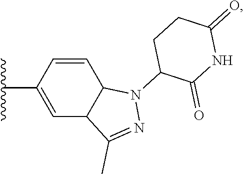 [SMILES: *C1=CC2C(C)=NN(C3CCC(=O)NC3=O)C2C=C1] | (ggg) |
| 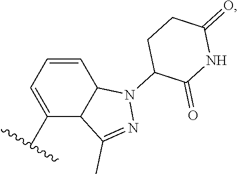 [SMILES: *C1=CC=CC2C1C(C)=NN2C1CCC(=O)NC1=O] | (hhh) |
| 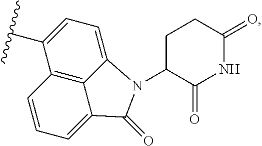 [SMILES: *c1ccc2c3c(cccc13)C(=O)N2C1CCC(=O)NC1=O] | (iii) |
| 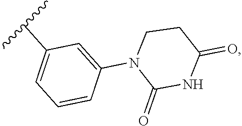 [SMILES: *c1cccc(N2CCC(=O)NC2=O)c1] | (jjj) |
| 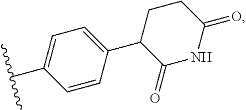 [SMILES: *c1ccc(C2CCC(=O)NC2=O)cc1] | (kkk) |
| 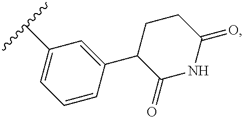 [SMILES: *c1cccc(C2CCC(=O)NC2=O)c1] | (lll) |
| 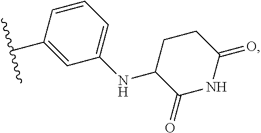 [SMILES: *c1cccc(NC2CCC(=O)NC2=O)c1] | (mmm) |
| 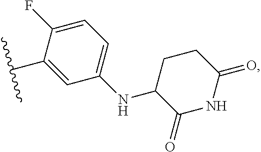 [SMILES: *c1cc(NC2CCC(=O)NC2=O)ccc1F] | (nnn) |
| 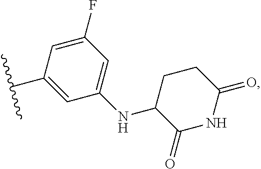 [SMILES: *c1cc(F)cc(NC2CCC(=O)NC2=O)c1] | (ooo) |
| 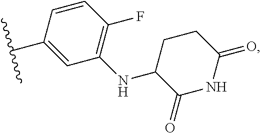 [SMILES: *c1ccc(F)c(NC2CCC(=O)NC2=O)c1] | (ppp) |
| 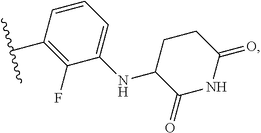 [SMILES: *c1cccc(NC2CCC(=O)NC2=O)c1F] | (qqq) |
| 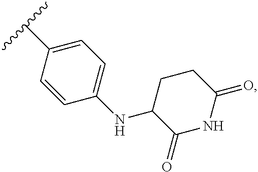 [SMILES: *c1ccc(NC2CCC(=O)NC2=O)cc1] | (rrr) |
| 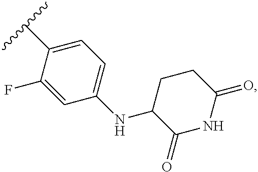 [SMILES: *c1ccc(NC2CCC(=O)NC2=O)cc1F] | (sss) |
| 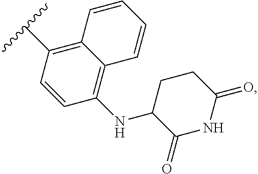 [SMILES: *c1ccc(NC2CCC(=O)NC2=O)c2ccccc12] | (ttt) |
| 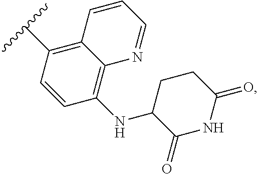 [SMILES: *c1ccc(NC2CCC(=O)NC2=O)c2ncccc12] | (uuu) |
| 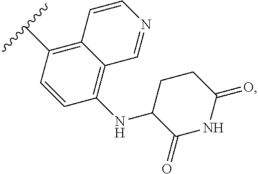 [SMILES: *c1ccc(NC2CCC(=O)NC2=O)c2cnccc12] | (vvv) |
| 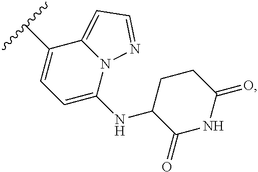 [SMILES: *c1ccc(NC2CCC(=O)NC2=O)n2nccc12] | (www) |
| 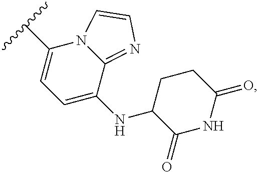 [SMILES: *c1ccc(NC2CCC(=O)NC2=O)c2nccn12] | (xxx) |
| 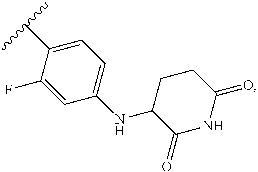 [SMILES: *c1ccc(NC2CCC(=O)NC2=O)cc1F] | (yyy) |
| 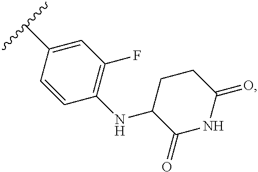 [SMILES: *c1ccc(NC2CCC(=O)NC2=O)c(F)c1] | (zzz) |
| 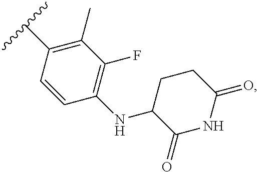 [SMILES: *c1ccc(NC2CCC(=O)NC2=O)c(F)c1C] | (aaaa) |
| 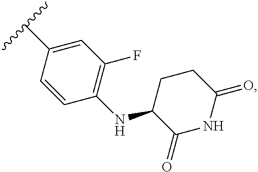 [SMILES: *c1ccc(N[C@H]2CCC(=O)NC2=O)c(F)c1] | (bbbb) |
| 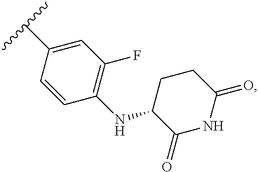 [SMILES: *c1ccc(N[C@@H]2CCC(=O)NC2=O)c(F)c1] | (cccc) |
| 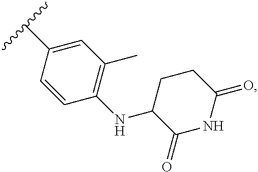 [SMILES: *c1ccc(NC2CCC(=O)NC2=O)c(C)c1] | (dddd) |
| 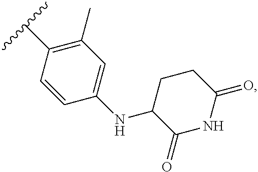 [SMILES: *c1ccc(NC2CCC(=O)NC2=O)cc1C] | (eeee) |
| 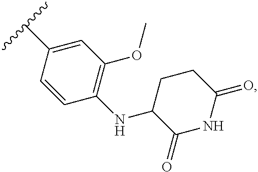 [SMILES: *c1ccc(NC2CCC(=O)NC2=O)c(OC)c1] | (ffff) |
| 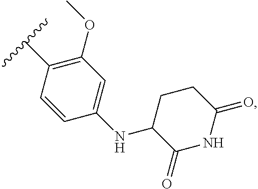 [SMILES: *c1ccc(NC2CCC(=O)NC2=O)cc1OC] | (gggg) |
| 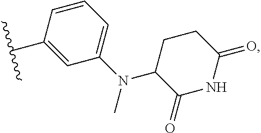 [SMILES: *c1cccc(N(C)C2CCC(=O)NC2=O)c1] | (hhhh) |
| 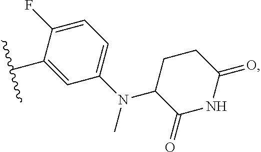 [SMILES: *c1cc(N(C)C2CCC(=O)NC2=O)ccc1F] | (iiii) |
| 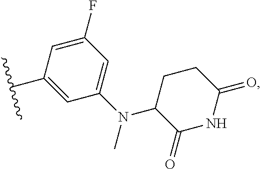 [SMILES: *c1cc(F)cc(N(C)C2CCC(=O)NC2=O)c1] | (jjjj) |
| 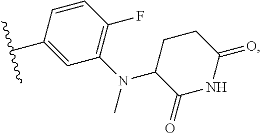 [SMILES: *c1ccc(F)c(N(C)C2CCC(=O)NC2=O)c1] | (kkkk) |
| 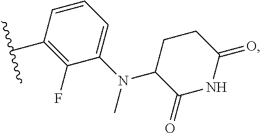 [SMILES: *c1cccc(N(C)C2CCC(=O)NC2=O)c1F] | (llll) |
| 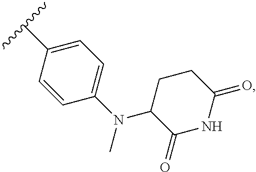 [SMILES: *c1ccc(N(C)C2CCC(=O)NC2=O)cc1] | (mmmm) |
| 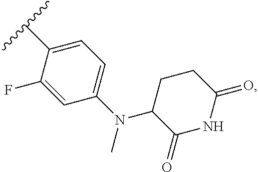 [SMILES: *c1ccc(N(C)C2CCC(=O)NC2=O)cc1F] | (nnnn) |
| 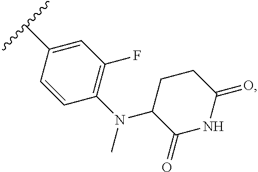 [SMILES: *c1ccc(N(C)C2CCC(=O)NC2=O)c(F)c1] | (oooo) |
| 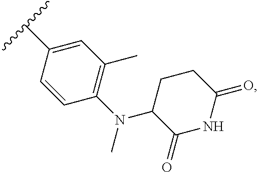 [SMILES: *c1ccc(N(C)C2CCC(=O)NC2=O)c(C)c1] | (pppp) |
| 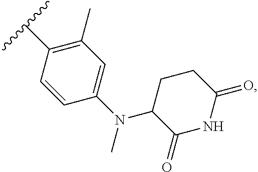 [SMILES: *c1ccc(N(C)C2CCC(=O)NC2=O)cc1C] | (qqqq) |
| 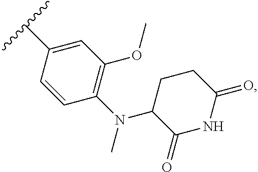 [SMILES: *c1ccc(N(C)C2CCC(=O)NC2=O)c(OC)c1] | (rrrr) |
| 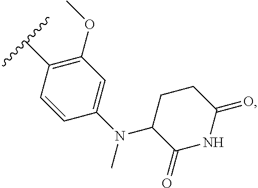 [SMILES: *c1ccc(N(C)C2CCC(=O)NC2=O)cc1OC] | (ssss) |
| 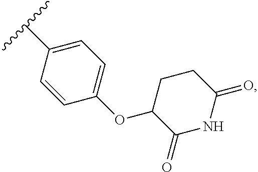 [SMILES: *c1ccc(OC2CCC(=O)NC2=O)cc1] | (tttt) |
| 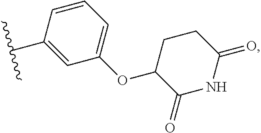 [SMILES: *c1cccc(OC2CCC(=O)NC2=O)c1] | (uuuu) |
| 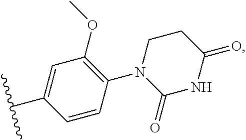 [SMILES: *c1ccc(N2CCC(=O)NC2=O)c(OC)c1] | (vvvv) |
| 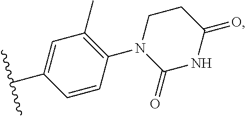 [SMILES: *c1ccc(N2CCC(=O)NC2=O)c(C)c1] | (wwww) |
| 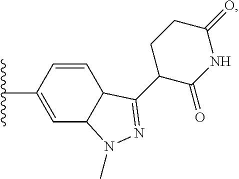 [SMILES: *C1=CC2C(C=C1)C(C1CCC(=O)NC1=O)=NN2C] | (xxxx) |
| 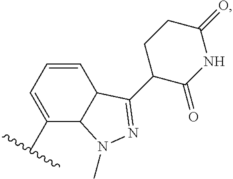 [SMILES: *C1=CC=CC2C(C3CCC(=O)NC3=O)=NN(C)C12] | (yyyy) |
| 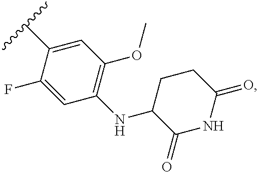 [SMILES: *c1cc(OC)c(NC2CCC(=O)NC2=O)cc1F] | (zzzz) |
| 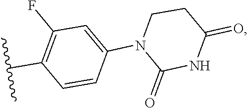 [SMILES: *c1ccc(N2CCC(=O)NC2=O)cc1F] | (aaaaa) |
| 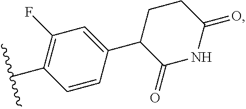 [SMILES: *c1ccc(C2CCC(=O)NC2=O)cc1F] | (bbbbb) (cccccc) |
| 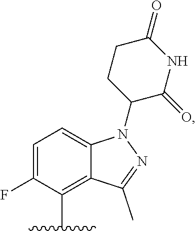 [SMILES: *c1c(F)ccc2c1c(C)nn2C1CCC(=O)NC1=O] | |
| or any of the LBM disclosed herein. | |

TABLE B

| Exemplified Linkers (L) | |
|---|---|
| 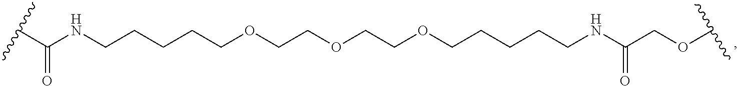 [SMILES: *OCC(=O)NCCCCCOCCOCCOCCCCCNC(*)=O] | (1) |
| 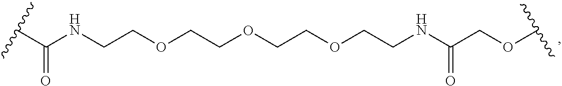 [SMILES: *OCC(=O)NCCOCCOCCOCCNC(*)=O ; ","] | (2) |
| 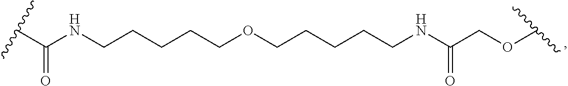 [SMILES: *OCC(=O)NCCCCCOCCCCCNC(*)=O] | (3) |
| 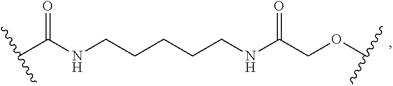 [SMILES: *OCC(=O)NCCCCCNC(*)=O] | (4) |
| 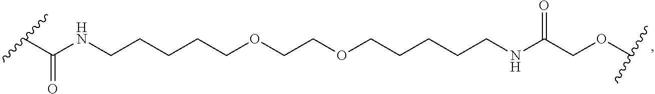 [SMILES: *OCC(=O)NCCCCCOCCOCCCCCNC(*)=O] | (5) |
| 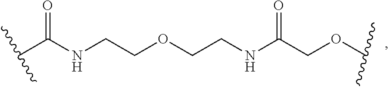 [SMILES: *OCC(=O)NCCOCCNC(*)=O] | (6) |
| 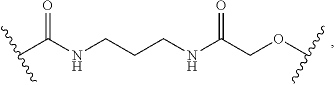 [SMILES: *OCC(=O)NCCCNC(*)=O] | (7) |
| 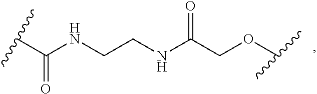 [SMILES: *OCC(=O)NCCNC(*)=O] | (8) |
| 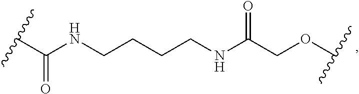 [SMILES: *OCC(=O)NCCCCNC(*)=O] | (9) |
| 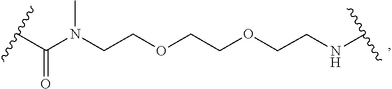 [SMILES: *NCCOCCOCCN(C)C(*)=O] | (10) |
| 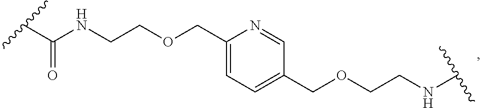 [SMILES: *NCCOCc1ccc(COCCNC(*)=O)nc1] | (11) |
| 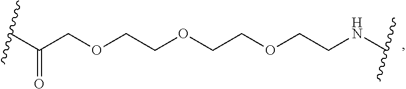 [SMILES: *NCCOCCOCCOCC(*)=O] | (12) |
| 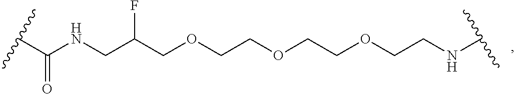 [SMILES: *NCCOCCOCCOCC(F)CNC(*)=O] | (13) |
| 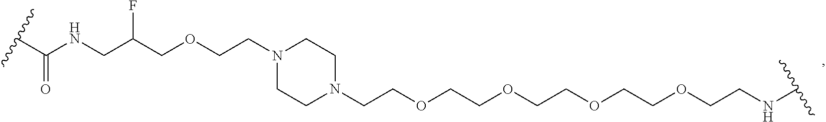 [SMILES: *NCCOCCOCCOCCOCCN1CCN(CCOCC(F)CNC(*)=O)CC1] | (14) |
| 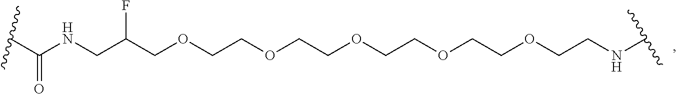 [SMILES: *NCCOCCOCCOCCOCCOCC(F)CNC(*)=O] | (15) |
| 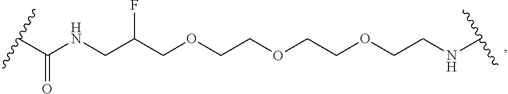 [SMILES: *NCCOCCOCCOCC(F)CNC(*)=O] | (16) |
| 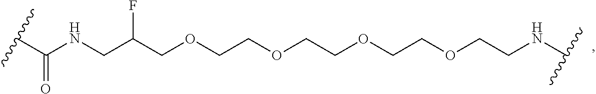 [SMILES: *NCCOCCOCCOCCOCC(F)CNC(*)=O] | (17) |
| 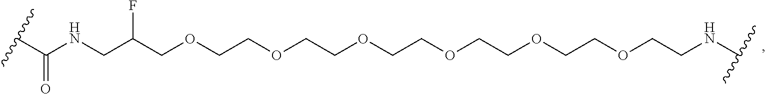 [SMILES: *NCCOCCOCCOCCOCCOCCOCC(F)CNC(*)=O] | (18) |
| 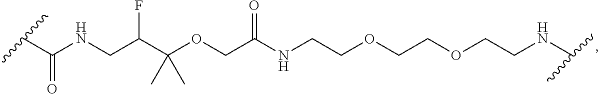 [SMILES: *NCCOCCOCCNC(=O)COC(C)(C)C(F)CNC(*)=O] | (19) |
| 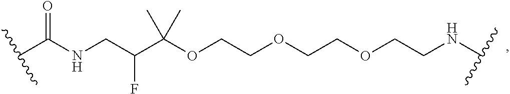 [SMILES: *NCCOCCOCCOC(C)(C)C(F)CNC(*)=O] | (20) |
| 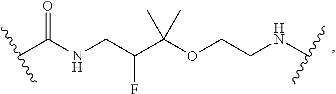 [SMILES: *NCCOC(C)(C)C(F)CNC(*)=O] | (21) |
| 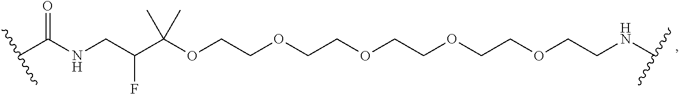 [SMILES: *NCCOCCOCCOCCOCCOC(C)(C)C(F)CNC(*)=O] | (22) |
| 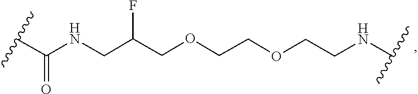 [SMILES: *NCCOCCOCC(F)CNC(*)=O] | (23) |
| 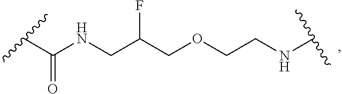 [SMILES: *NCCOCC(F)CNC(*)=O] | (24) |
| 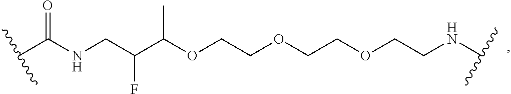 [SMILES: *NCCOCCOCCOC(C)C(F)CNC(*)=O] | (25) |
| 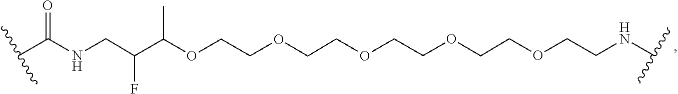 [SMILES: *NCCOCCOCCOCCOCCOC(C)C(F)CNC(*)=O] | (26) |
| 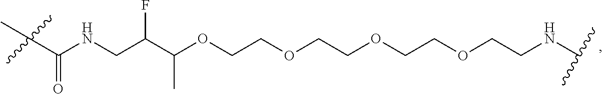 [SMILES: *NCCOCCOCCOCCOC(C)C(F)CNC(*)=O] | (27) |
| 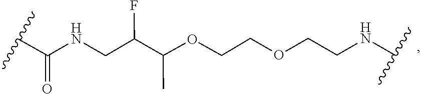 [SMILES: *NCCOCCOC(C)C(F)CNC(*)=O] | (28) |
| 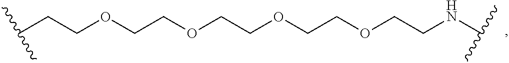 [SMILES: *CCOCCOCCOCCOCCN*] | (29) |
| 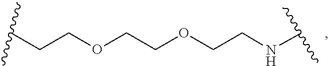 [SMILES: *CCOCCOCCN*] | (30) |
| 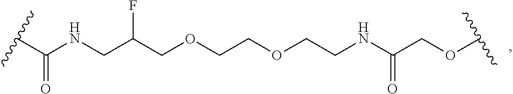 [SMILES: *OCC(=O)NCCOCCOCC(F)CNC(*)=O] | (31) |
| 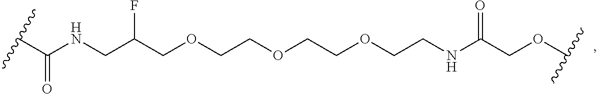 [SMILES: *OCC(=O)NCCOCCOCCOCC(F)CNC(*)=O] | (32) |
| 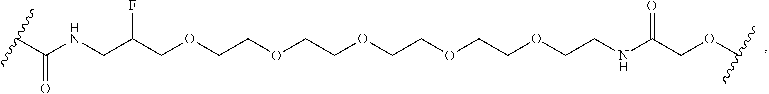 [SMILES: *OCC(=O)NCCOCCOCCOCCOCCOCC(F)CNC(*)=O] | (33) |
| 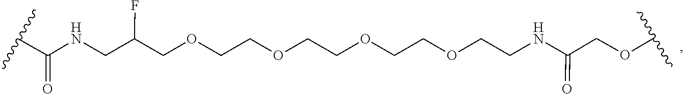 [SMILES: *OCC(=O)NCCOCCOCCOCCOCC(F)CNC(*)=O] | (34) |
| 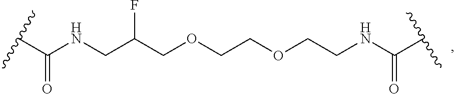 [SMILES: *C(=O)NCCOCCOCC(F)CNC(*)=O] | (35) |
| 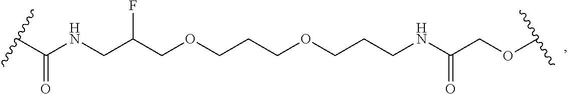 [SMILES: *OCC(=O)NCCCOCCCOCC(F)CNC(*)=O] | (36) |
| 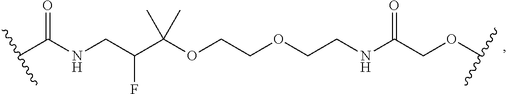 [SMILES: *OCC(=O)NCCOCCOC(C)(C)C(F)CNC(*)=O] | (37) |
| 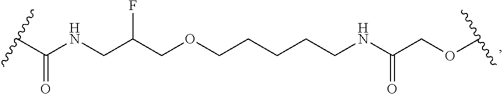 [SMILES: *OCC(=O)NCCCCCOCC(F)CNC(*)=O] | (38) |
| 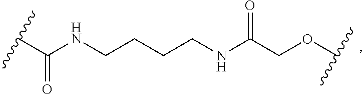 [SMILES: *OCC(=O)NCCCCNC(*)=O] | (39) |
| 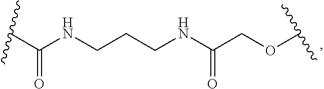 [SMILES: *OCC(=O)NCCCNC(*)=O] | (40) |
| 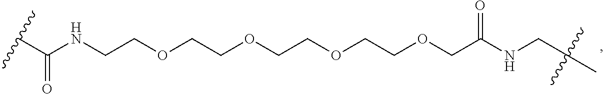 [SMILES: *CNC(=O)COCCOCCOCCOCCNC(*)=O] | (41) |
| 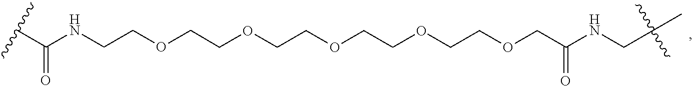 [SMILES: *CNC(=O)COCCOCCOCCOCCOCCNC(*)=O] | (42) |
| 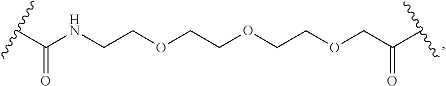 [SMILES: *C(=O)COCCOCCOCCNC(*)=O] | (43) |
| 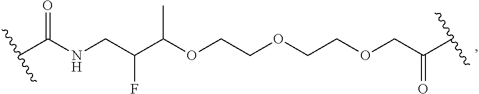 [SMILES: *C(=O)COCCOCCOC(C)C(F)CNC(*)=O] | (44) |
| 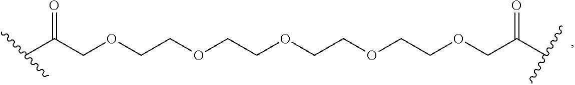 [SMILES: *C(=O)COCCOCCOCCOCCOCC(*)=O] | (45) |
| 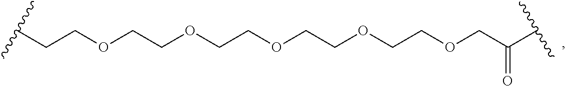 [SMILES: *CCOCCOCCOCCOCCOCC(*)=O] | (46) |
| 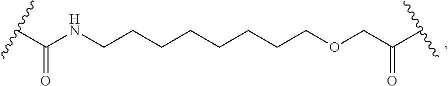 [SMILES: *C(=O)COCCCCCCCCNC(*)=O] | (47) |
| 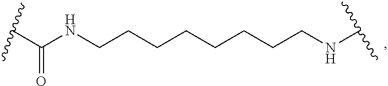 [SMILES: *NCCCCCCCCNC(*)=O] | (49) |
| 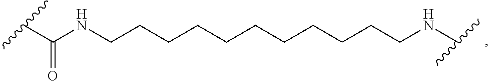 [SMILES: *NCCCCCCCCCCCNC(*)=O] | (50) |
| 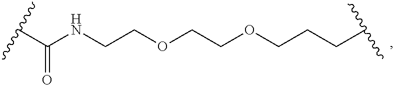 [SMILES: *CCCOCCOCCNC(*)=O] | (51) |
| 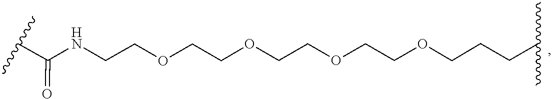 [SMILES: *CCCOCCOCCOCCOCCNC(*)=O] | (52) |
| 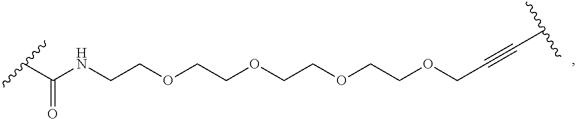 [SMILES: *C#CCOCCOCCOCCOCCNC(*)=O] | (53) |
| 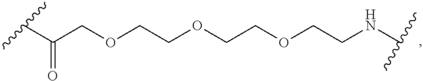 [SMILES: *NCCOCCOCCOCC(*)=O] | (54) |
| 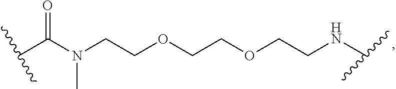 [SMILES: *NCCOCCOCCN(C)C(*)=O] | (55) |
| 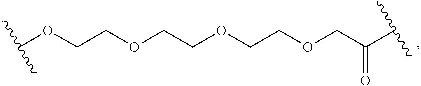 [SMILES: *OCCOCCOCCOCC(*)=O] | (56) |
| 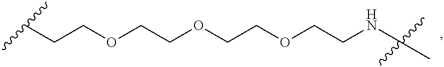 [SMILES: *CCOCCOCCOCCN*] | (57) |
| 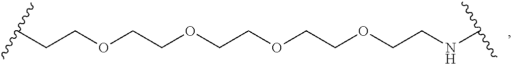 [SMILES: *CCOCCOCCOCCOCCN*] | (58) |
| 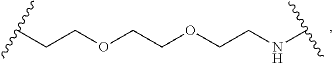 [SMILES: *CCOCCOCCN* ; ","] | (59) |
| 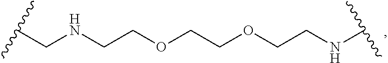 [SMILES: *CNCCOCCOCCN*] | (60) |
| 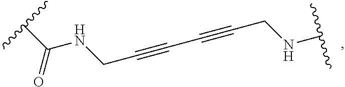 [SMILES: *NCC#CC#CCNC(*)=O] | (61) |
| 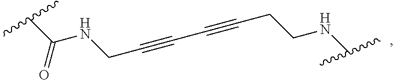 [SMILES: *NCCC#CC#CCNC(*)=O] | (62) |
| 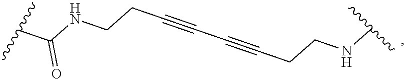 [SMILES: *NCCC#CC#CCCNC(*)=O] | (63) |
| 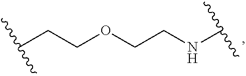 [SMILES: *CCOCCN*] | (64) |
| 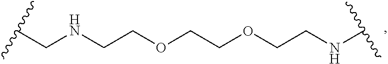 [SMILES: *CNCCOCCOCCN* ; ","] | (65) |
| 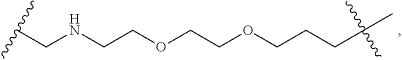 [SMILES: *CCCOCCOCCNC*] | (66) |
| 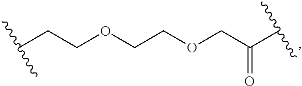 [SMILES: *CCOCCOCC(*)=O] | (67) |
| 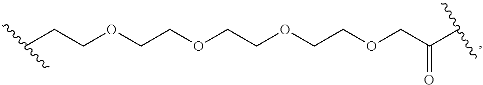 [SMILES: *CCOCCOCCOCCOCC(*)=O] | (68) |
| 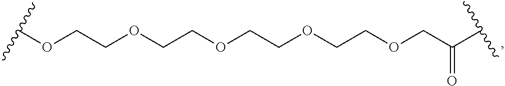 [SMILES: *OCCOCCOCCOCCOCC(*)=O] | (69) |
| 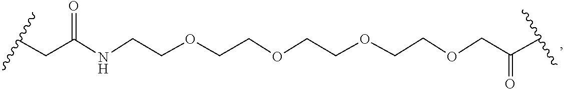 [SMILES: *CC(=O)NCCOCCOCCOCCOCC(*)=O] | (70) |
| 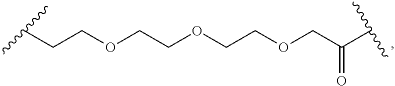 [SMILES: *CCOCCOCCOCC(*)=O] | (71) |
| 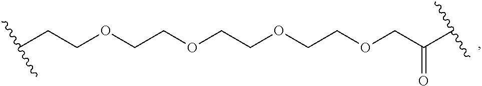 [SMILES: *CCOCCOCCOCCOCC(*)=O] | (72) |
| 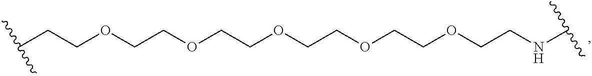 [SMILES: *CCOCCOCCOCCOCCOCCN*] | (73) |
| 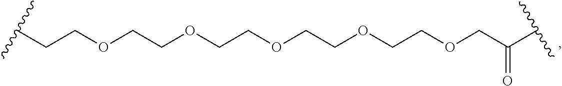 [SMILES: *CCOCCOCCOCCOCCOCC(*)=O] | (74) |
| 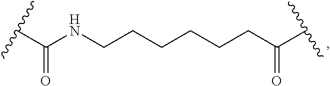 [SMILES: *C(=O)CCCCCCNC(*)=O] | (75) |
| 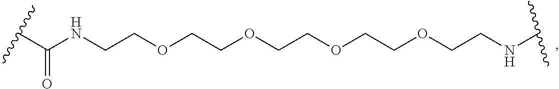 [SMILES: *NCCOCCOCCOCCOCCNC(*)=O] | (76) |
| 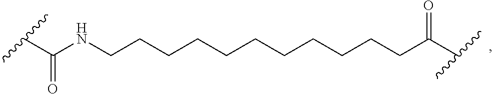 [SMILES: *C(=O)CCCCCCCCCCCNC(*)=O] | (77) |
| 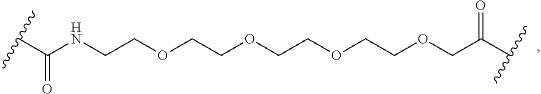 [SMILES: *C(=O)COCCOCCOCCOCCNC(*)=O] | (78) |
| 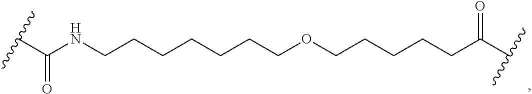 [SMILES: *C(=O)CCCCCOCCCCCCCNC(*)=O] | (79) |
| 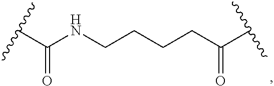 [SMILES: *C(=O)CCCCNC(*)=O] | (80) |
| 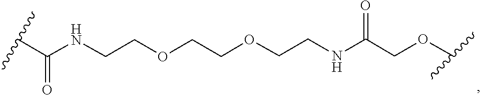 [SMILES: *OCC(=O)NCCOCCOCCNC(*)=O] | (81) |
| 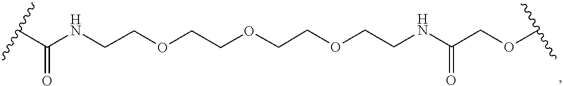 [SMILES: *OCC(=O)NCCOCCOCCOCCNC(*)=O] | (82) |
| 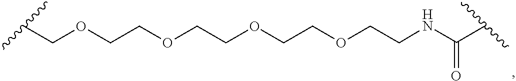 [SMILES: *COCCOCCOCCOCCNC(*)=O] | (83) |
| 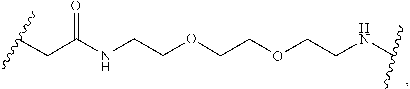 [SMILES: *CC(=O)NCCOCCOCCN*] | (84) |
| 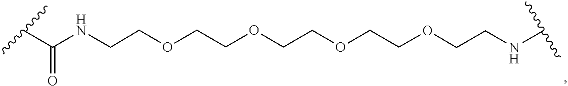 [SMILES: *NCCOCCOCCOCCOCCNC(*)=O] | (85) |
| 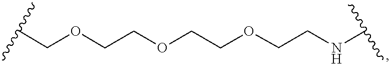 [SMILES: *COCCOCCOCCN*] | (86) |
| 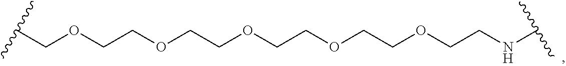 [SMILES: *COCCOCCOCCOCCOCCN*] | (87) |
| 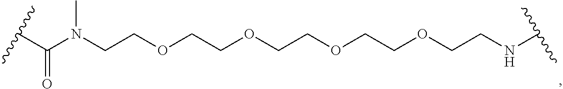 [SMILES: *NCCOCCOCCOCCOCCN(C)C(*)=O] | (88) |
| 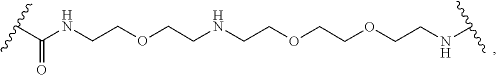 [SMILES: *NCCOCCOCCNCCOCCNC(*)=O] | (89) |
| 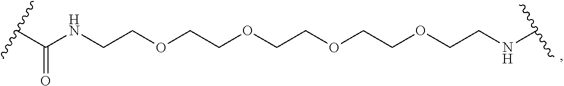 [SMILES: *NCCOCCOCCOCCOCCNC(*)=O] | (90) |
| 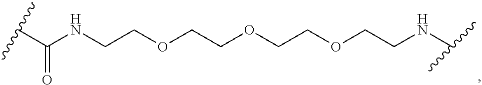 [SMILES: *NCCOCCOCCOCCNC(*)=O] | (91) |
| 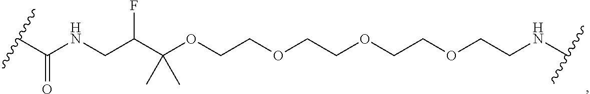 [SMILES: *NCCOCCOCCOCCOC(C)(C)C(F)CNC(*)=O ; ","] | (92) |
| 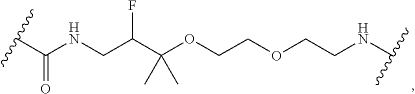 [SMILES: *NCCOCCOC(C)(C)C(F)CNC(*)=O] | (93) |
| 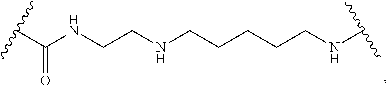 [SMILES: *NCCCCCNCCNC(*)=O] | (94) |
| 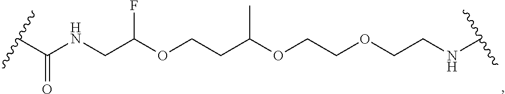 [SMILES: *NCCOCCOC(C)CCOC(F)CNC(*)=O ; ","] | (95) |
| 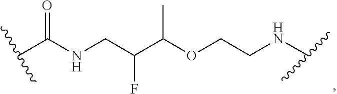 [SMILES: *NCCOC(C)C(F)CNC(*)=O] | (96) |
| 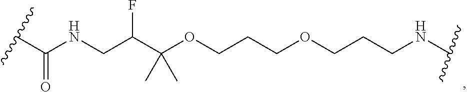 [SMILES: *NCCCOCCCOC(C)(C)C(F)CNC(*)=O] | (97) |
| 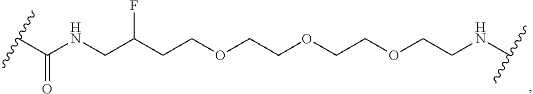 [SMILES: *NCCOCCOCCOCCC(F)CNC(*)=O ; ","] | (98) |
| 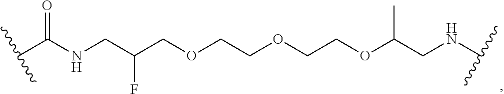 [SMILES: *NCC(C)OCCOCCOCC(F)CNC(*)=O] | (99) |
| 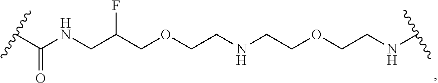 [SMILES: *NCCOCCNCCOCC(F)CNC(*)=O] | (100) |
| 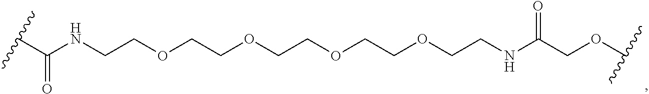 [SMILES: *OCC(=O)NCCOCCOCCOCCOCCNC(*)=O] | (101) |
| 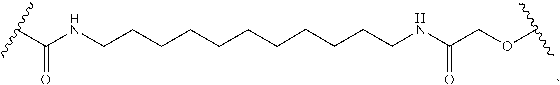 [SMILES: *OCC(=O)NCCCCCCCCCCCNC(*)=O] | (102) |
| 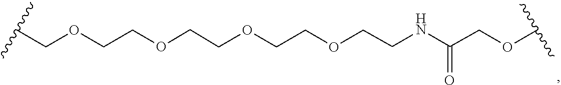 [SMILES: *COCCOCCOCCOCCNC(=O)CO*] | (103) |
| 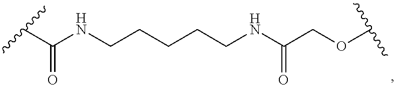 [SMILES: *OCC(=O)NCCCCCNC(*)=O] | (104) |
| 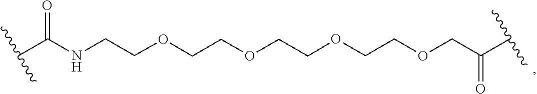 [SMILES: *C(=O)COCCOCCOCCOCCNC(*)=O] | (105) |
| 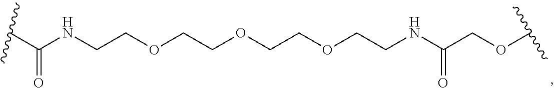 [SMILES: *OCC(=O)NCCOCCOCCOCCNC(*)=O] | (106) |
| 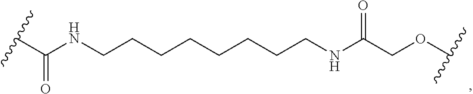 | (107) |
| 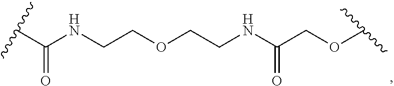 [SMILES: *OCC(=O)NCCOCCNC(*)=O] | (108) |
| 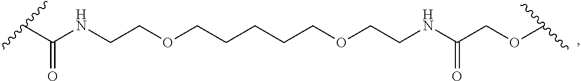 [SMILES: *OCC(=O)NCCOCCCCCOCCNC(*)=O] | (109) |
| 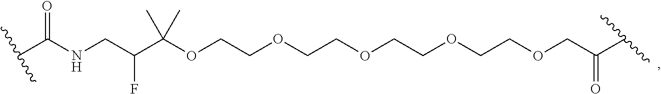 [SMILES: *C(=O)COCCOCCOCCOCCOC(C)(C)C(F)CNC(*)=O] | (110) |
| 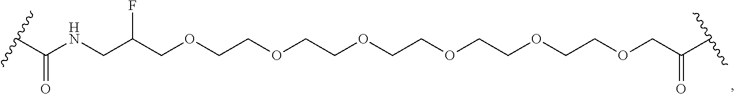 [SMILES: *C(=O)COCCOCCOCCOCCOCCOCC(F)CNC(*)=O] | (111) |
| 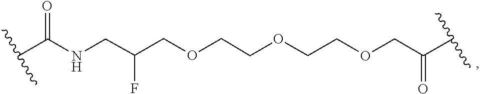 [SMILES: *C(=O)COCCOCCOCC(F)CNC(*)=O] | (112) |
| 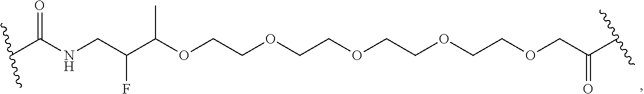 [SMILES: *C(=O)COCCOCCOCCOCCOC(C)C(F)CNC(*)=O] | (113) |
| 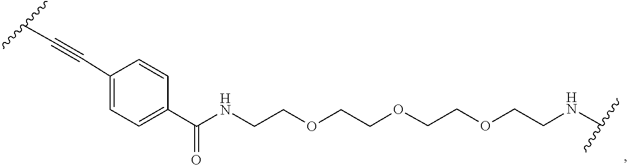 [SMILES: *C#Cc1ccc(C(=O)NCCOCCOCCOCCN*)cc1] | (114) |
| 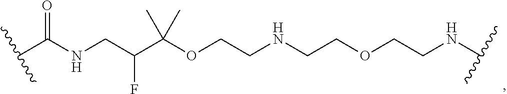 [SMILES: *NCCOCCNCCOC(C)(C)C(F)CNC(*)=O] | (115) |
| 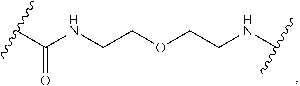 [SMILES: *NCCOCCNC(*)=O] | (116) |
| 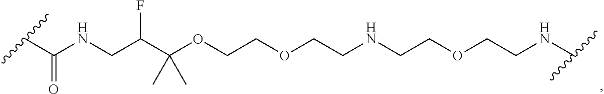 [SMILES: *NCCOCCNCCOCCOC(C)(C)C(F)CNC(*)=O] | (117) |
| 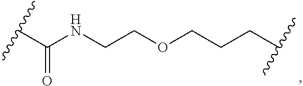 [SMILES: *CCCOCCNC(*)=O] | (118) |
| 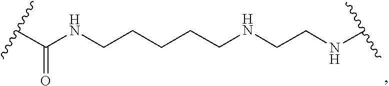 [SMILES: *NCCNCCCCCNC(*)=O] | |
| 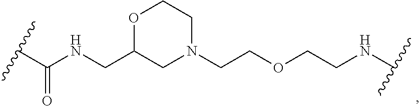 [SMILES: *NCCOCCN1CCOC(CNC(*)=O)C1] | (119) |
| 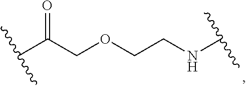 [SMILES: *NCCOCC(*)=O] | |
| 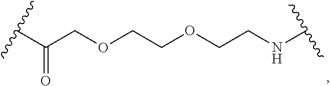 [SMILES: *NCCOCCOCC(*)=O] | (120) |
| 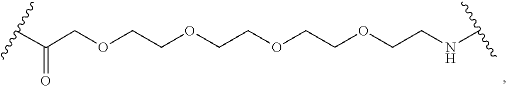 [SMILES: *NCCOCCOCCOCCOCC(*)=O] | (121) |
| 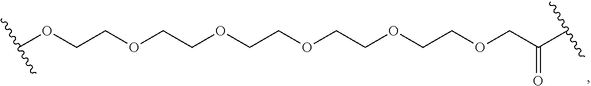 [SMILES: *OCCOCCOCCOCCOCCOCC(*)=O] | (122) |
| 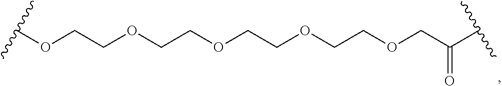 [SMILES: *OCCOCCOCCOCCOCC(*)=O] | (123) |
| 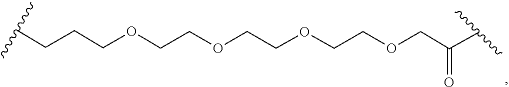 [SMILES: *CCCOCCOCCOCCOCC(*)=O] | (124) |
| 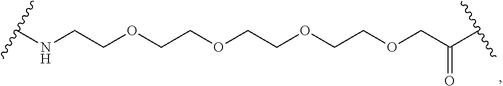 [SMILES: *NCCOCCOCCOCCOCC(*)=O] | (125) |
| 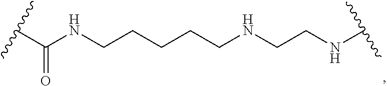 [SMILES: *NCCNCCCCCNC(*)=O] | (126) |
| 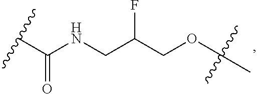 [SMILES: *OCC(F)CNC(*)=O] | |
| 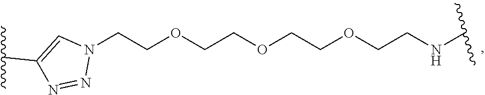 [SMILES: *NCCOCCOCCOCCn1cc(*)nn1] | (127) |
| 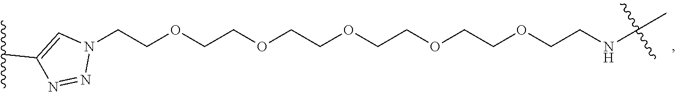 [SMILES: *NCCOCCOCCOCCOCCOCCn1cc(*)nn1] | (128) |
| 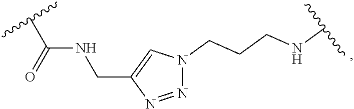 [SMILES: *NCCCn1cc(CNC(*)=O)nn1] | (129) |
| 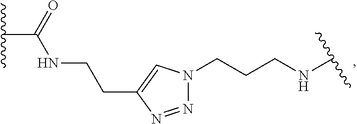 [SMILES: *NCCCn1cc(CCNC(*)=O)nn1] | (130) |
| 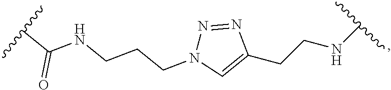 [SMILES: *NCCc1cn(CCCNC(*)=O)nn1] | (131) |
| 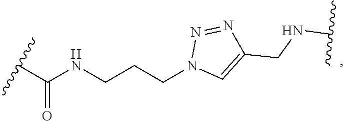 [SMILES: *NCc1cn(CCCNC(*)=O)nn1] | (132) |
| 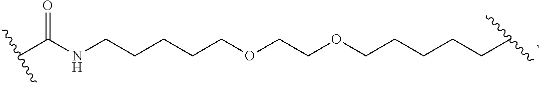 [SMILES: *CCCCCOCCOCCCCCNC(*)=O] | (133) |
| 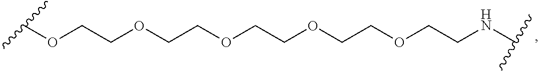 [SMILES: *NCCOCCOCCOCCOCCO*] | (134) |
| 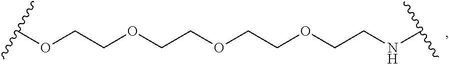 [SMILES: *NCCOCCOCCOCCO*] | (135) |
| 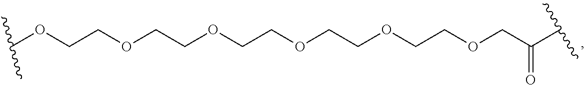 [SMILES: *OCCOCCOCCOCCOCCOCC(*)=O] | (136) |
| 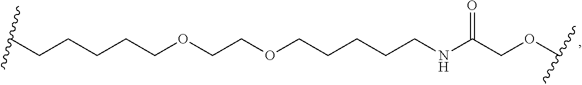 [SMILES: *CCCCCOCCOCCCCCNC(=O)CO*] | (137) |
| 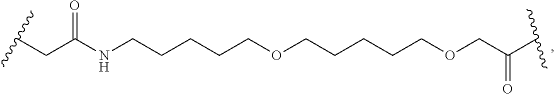 [SMILES: *CC(=O)NCCCCCOCCCCCOCC(*)=O] | (138) |
| 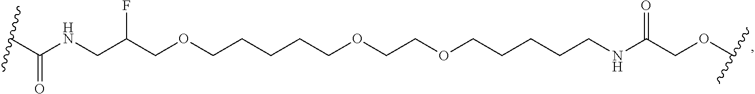 [SMILES: *OCC(=O)NCCCCCOCCOCCCCCOCC(F)CNC(*)=O] | (139) |
| 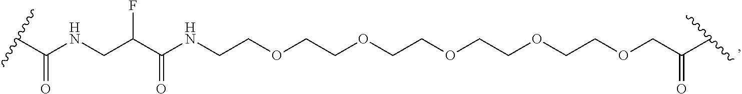 [SMILES: *C(=O)COCCOCCOCCOCCOCCNC(=O)C(F)CNC(*)=O] | (140) |
| 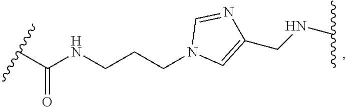 [SMILES: *NCc1cn(CCCNC(*)=O)cn1] | (141) |
| 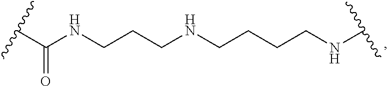 [SMILES: *NCCCCNCCCNC(*)=O] | (142) |
| 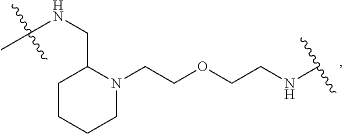 [SMILES: *NCCOCCN1CCCCC1CN* ; ","] | (143) |
| 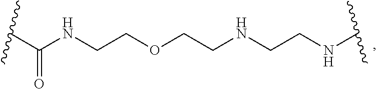 [SMILES: *NCCNCCOCCNC(*)=O] | (144) |
| 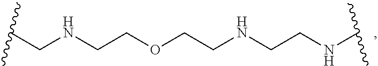 [SMILES: *CNCCOCCNCCN*] | (145) |
| 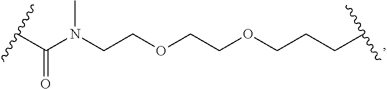 [SMILES: *CCCOCCOCCN(C)C(*)=O] | (146) |
| 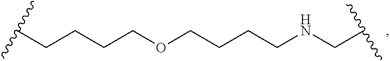 [SMILES: *CCCCOCCCCNC*] | (147) |
| 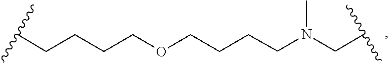 [SMILES: *CCCCOCCCCN(C)C*] | (148) |
| 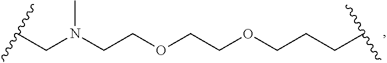 [SMILES: *CCCOCCOCCN(C)C*] | (149) |
| 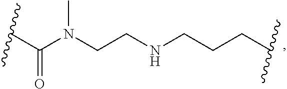 [SMILES: *CCCNCCN(C)C(*)=O] | (150) |
| 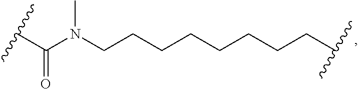 [SMILES: *CCCCCCCCN(C)C(*)=O ; ","] | (151) |
| 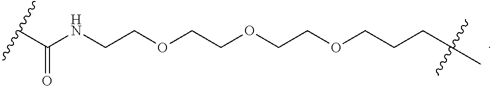 [SMILES: *CCCOCCOCCOCCNC(*)=O] | (152) |
| 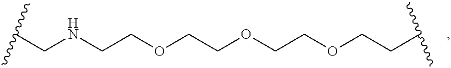 [SMILES: *CCOCCOCCOCCNC*] | (153) |
| 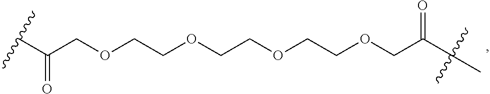 [SMILES: *C(=O)COCCOCCOCCOCC(*)=O] | (154) |
| 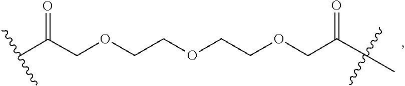 [SMILES: *C(=O)COCCOCCOCC(*)=O] | (155) |
| 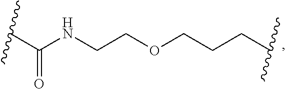 [SMILES: *CCCOCCNC(*)=O] | (156) |
| 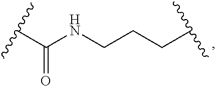 [SMILES: *CCCNC(*)=O] | (157) |
| 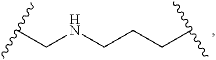 [SMILES: *CCCNC*] | (158) |
| 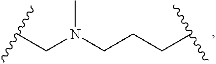 [SMILES: *CCCN(C)C*] | (159) |
| 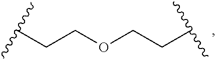 [SMILES: *CCOCC*] | (160) |
| 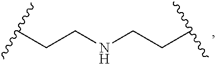 [SMILES: *CCNCC*] | (161) |
| 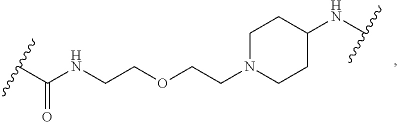 [SMILES: *NC1CCN(CCOCCNC(*)=O)CC1] | (162) |
| 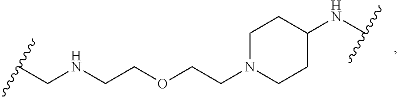 [SMILES: *CNCCOCCN1CCC(N*)CC1] | (163) |
| 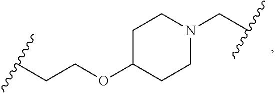 [SMILES: *CCOC1CCN(C*)CC1] | (164) |
| 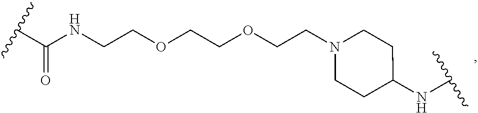 [SMILES: *NC1CCN(CCOCCOCCNC(*)=O)CC1] | (165) |
| 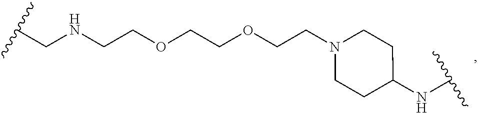 [SMILES: *CNCCOCCOCCN1CCC(N*)CC1] | (166) |
| 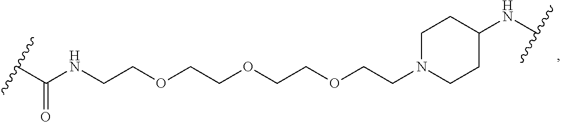 [SMILES: *NC1CCN(CCOCCOCCOCCNC(*)=O)CC1 ; ","] | (167) |
| 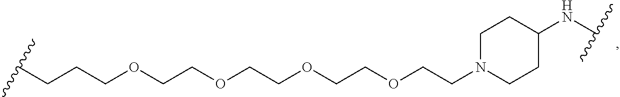 [SMILES: *CCCOCCOCCOCCOCCN1CCC(N*)CC1 ; ","] | (168) |
| 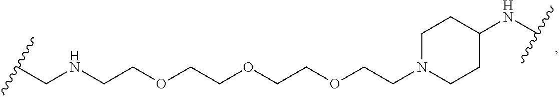 [SMILES: *CNCCOCCOCCOCCN1CCC(N*)CC1] | (169) |
| 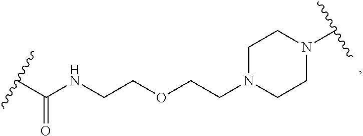 [SMILES: *C(=O)NCCOCCN1CCN(*)CC1] | (170) |
| 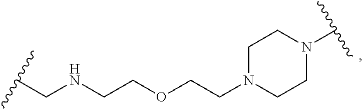 [SMILES: *CNCCOCCN1CCN(*)CC1] | (171) |
| 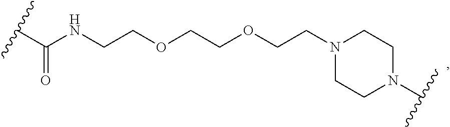 [SMILES: *C(=O)NCCOCCOCCN1CCN(*)CC1] | (172) |
| 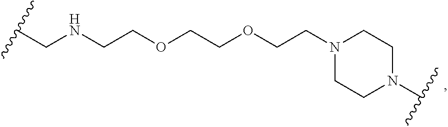 [SMILES: *CNCCOCCOCCN1CCN(*)CC1] | (173) |
| 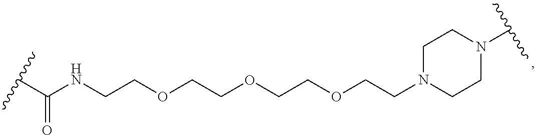 [SMILES: *C(=O)NCCOCCOCCOCCN1CCN(*)CC1 ; ","] | (174) |
| 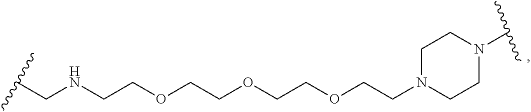 [SMILES: *CNCCOCCOCCOCCN1CCN(*)CC1] | (175) |
| 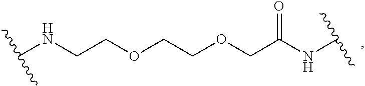 [SMILES: *NCCOCCOCC(=O)N*] | (176) |
| 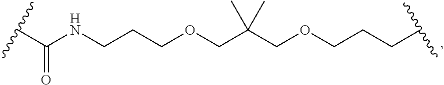 [SMILES: *CCCOCC(C)(C)COCCCNC(*)=O] | (177) |
| 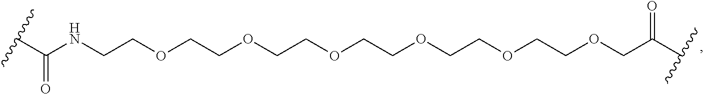 [SMILES: *C(=O)COCCOCCOCCOCCOCCOCCNC(*)=O] | (178) |
| 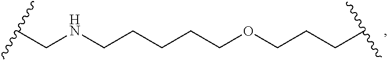 [SMILES: *CCCOCCCCCNC*] | (179) |
| 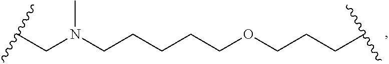 [SMILES: *CCCOCCCCCN(C)C*] | (180) |
| 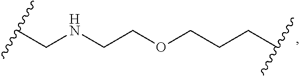 [SMILES: *CCCOCCNC*] | (181) |
| 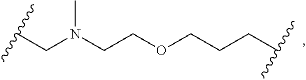 [SMILES: *CCCOCCN(C)C*] | (182) |
| 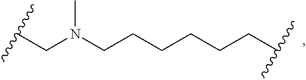 [SMILES: *CCCCCCN(C)C*] | (183) |
| 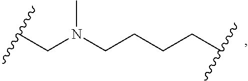 [SMILES: *CCCCN(C)C*] | (184) |
| 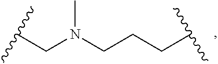 [SMILES: *CCCN(C)C*] | (185) |
| 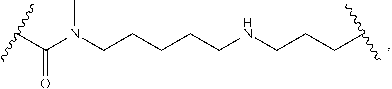 [SMILES: *CCCNCCCCCN(C)C(*)=O] | (186) |
| 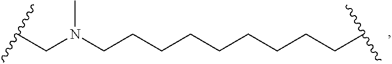 [SMILES: *CCCCCCCCCN(C)C*] | (187) |
| 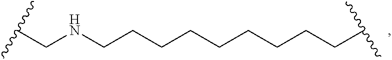 [SMILES: *CCCCCCCCCNC*] | (188) |
| 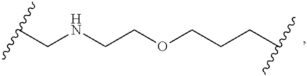 [SMILES: *CCCOCCNC*] | (189) |
| 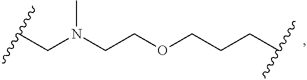 [SMILES: *CCCOCCN(C)C*] | (190) |
| 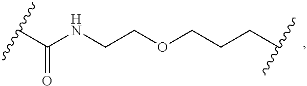 [SMILES: *CCCOCCNC(*)=O] | (191) |
| 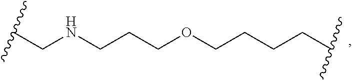 [SMILES: *CCCCOCCCNC*] | (192) |
| 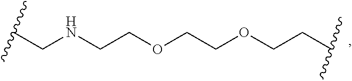 [SMILES: *CCOCCOCCNC*] | (193) |
| 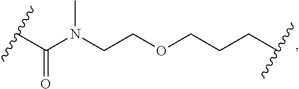 [SMILES: *CCCOCCN(C)C(*)=O] | (194) |
| 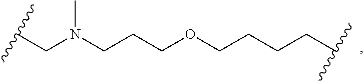 [SMILES: *CCCCOCCCN(C)C*] | (195) |
| 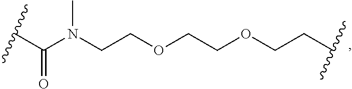 [SMILES: *CCOCCOCCN(C)C(*)=O] | (196) |
| 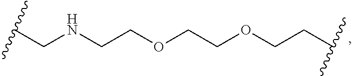 [SMILES: *CCOCCOCCNC*] | (197) |
| 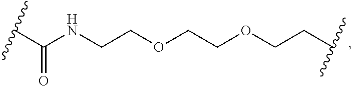 [SMILES: *CCOCCOCCNC(*)=O] | (198) |
| 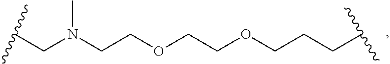 [SMILES: *CCCOCCOCCN(C)C*] | (199) |
| 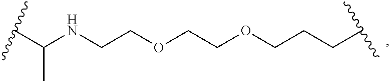 [SMILES: *CCCOCCOCCNC(*)C] | (200) |
| 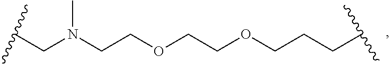 [SMILES: *CCCOCCOCCN(C)C*] | (201) |
| 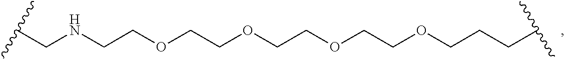 [SMILES: *CCCOCCOCCOCCOCCNC*] | (202) |
| 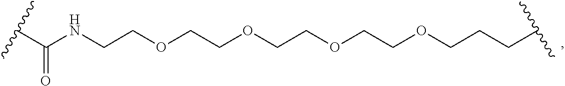 [SMILES: *CCCOCCOCCOCCOCCNC(*)=O] | (203) |
| 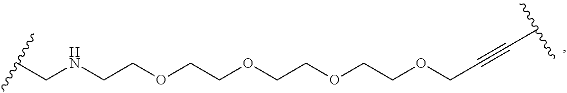 [SMILES: *C#CCOCCOCCOCCOCCNC*] | (204) |
| 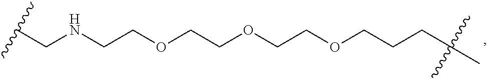 [SMILES: *CCCOCCOCCOCCNC*] | (205) |
| 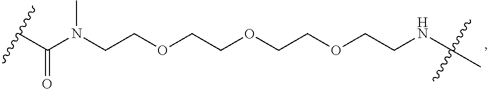 [SMILES: *NCCOCCOCCOCCN(C)C(*)=O] | (206) |
| 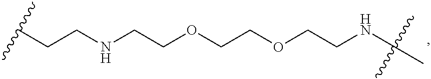 [SMILES: *CCNCCOCCOCCN*] | (207) |
| 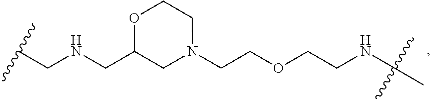 [SMILES: *CNCC1CN(CCOCCN*)CCO1] | (208) |
| 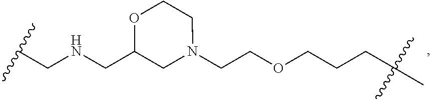 [SMILES: *CCCOCCN1CCOC(CNC*)C1] | (209) |
| 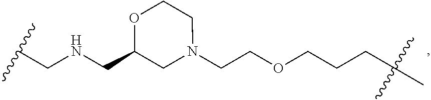 [SMILES: *CCCOCCN1CCO[C@H](CNC*)C1] | (210) |
| 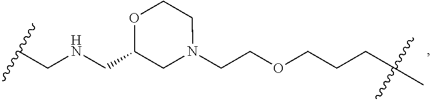 [SMILES: *CCCOCCN1CCO[C@@H](CNC*)C1] | (211) |
| 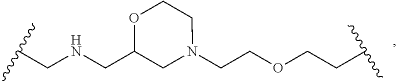 [SMILES: *CCOCCN1CCOC(CNC*)C1] | (212) |
| 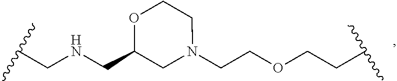 [SMILES: *CCOCCN1CCO[C@H](CNC*)C1] | (213) |
| 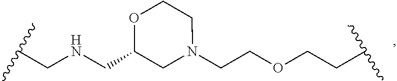 [SMILES: *CCOCCN1CCO[C@@H](CNC*)C1] | (214) |
| 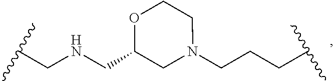 [SMILES: *CCCN1CCO[C@@H](CNC*)C1] | (215) |
| 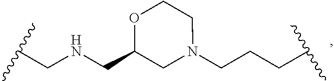 [SMILES: *CCCN1CCO[C@H](CNC*)C1] | (216) |
| 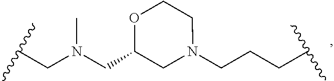 [SMILES: *CCCN1CCO[C@@H](CN(C)C*)C1] | (217) |
| 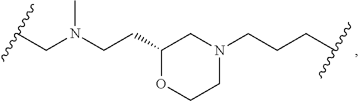 [SMILES: *CCCN1CCO[C@H](CCN(C)C*)C1] | (218) |
| 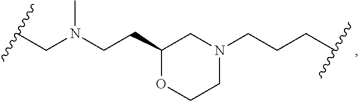 [SMILES: *CCCN1CCO[C@@H](CCN(C)C*)C1] | (219) |
| 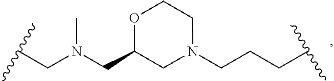 [SMILES: *CCCN1CCO[C@H](CN(C)C*)C1] | (220) |
| 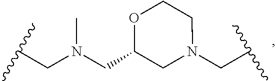 [SMILES: *CN(C)C[C@H]1CN(C*)CCO1] | (221) |
| 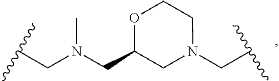 [SMILES: *CN(C)C[C@@H]1CN(C*)CCO1] | (222) |
| 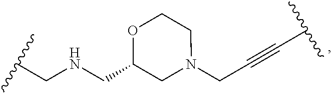 [SMILES: *C#CCN1CCO[C@@H](CNC*)C1] | (223) |
| 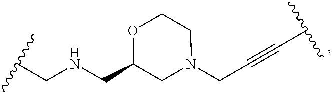 [SMILES: *C#CCN1CCO[C@H](CNC*)C1] | (224) |
| 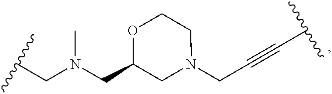 [SMILES: *C#CCN1CCO[C@H](CN(C)C*)C1] | (225) |
| 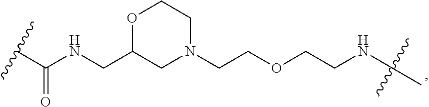 [SMILES: *NCCOCCN1CCOC(CNC(*)=O)C1] | (226) |
| 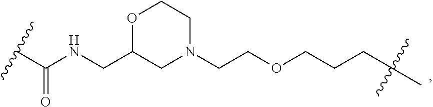 [SMILES: *CCCOCCN1CCOC(CNC(*)=O)C1] | (227) |
| 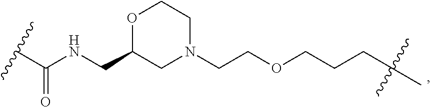 [SMILES: *CCCOCCN1CCO[C@H](CNC(*)=O)C1] | (228) |
| 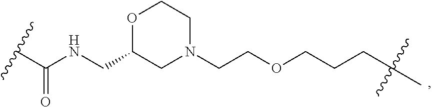 [SMILES: *CCCOCCN1CCO[C@@H](CNC(*)=O)C1] | (229) |
| 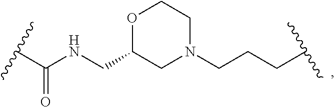 [SMILES: *CCCN1CCO[C@@H](CNC(*)=O)C1] | (230) |
| 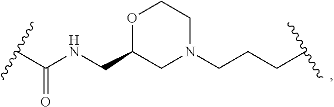 [SMILES: *CCCN1CCO[C@H](CNC(*)=O)C1] | (231) |
| 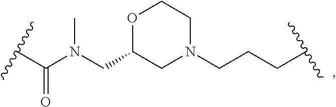 [SMILES: *CCCN1CCO[C@@H](CN(C)C(*)=O)C1] | (232) |
| 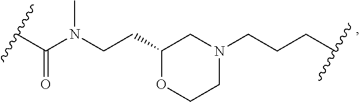 [SMILES: *CCCN1CCO[C@H](CCN(C)C(*)=O)C1] | (233) |
| 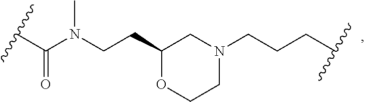 [SMILES: *CCCN1CCO[C@@H](CCN(C)C(*)=O)C1] | (234) |
| 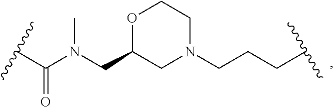 [SMILES: *CCCN1CCO[C@H](CN(C)C(*)=O)C1] | (235) |
| 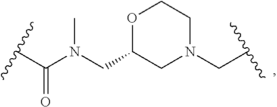 [SMILES: *CN1CCO[C@@H](CN(C)C(*)=O)C1] | (236) |
| 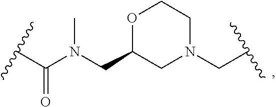 [SMILES: *CN1CCO[C@H](CN(C)C(*)=O)C1] | (237) |
| 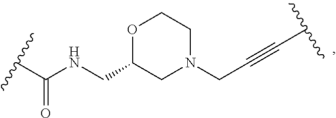 | (238) |
| 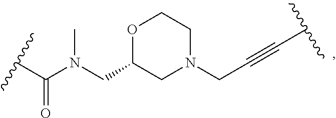 | (239) |
| 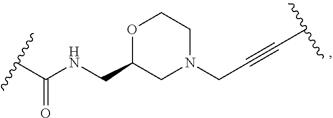 | (240) |
| 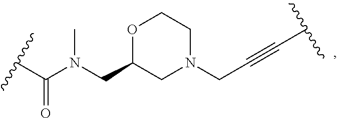 | (241) |
| 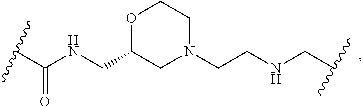 [SMILES: *CNCCN1CCO[C@@H](CNC(*)=O)C1] | (242) |
| 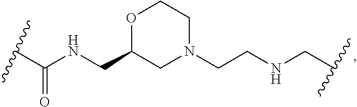 [SMILES: *CNCCN1CCO[C@H](CNC(*)=O)C1] | (243) |
| 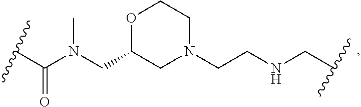 [SMILES: *CNCCN1CCO[C@@H](CN(C)C(*)=O)C1] | (244) |
| 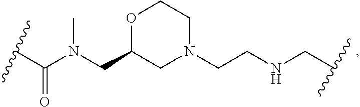 [SMILES: *CNCCN1CCO[C@H](CN(C)C(*)=O)C1] | (245) |
| 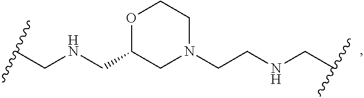 [SMILES: *CNCCN1CCO[C@@H](CNC*)C1] | (246) |
| 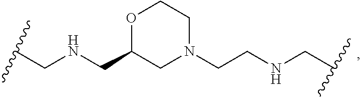 [SMILES: *CNCCN1CCO[C@H](CNC*)C1] | (247) |
| 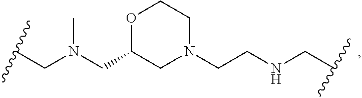 [SMILES: *CNCCN1CCO[C@@H](CN(C)C*)C1] | (248) |
| 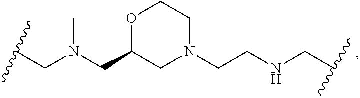 [SMILES: *CNCCN1CCO[C@H](CN(C)C*)C1] | (249) |
| 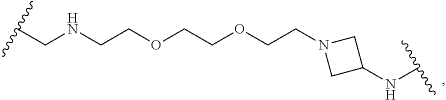 [SMILES: *CNCCOCCOCCN1CC(N*)C1] | (250) |
| 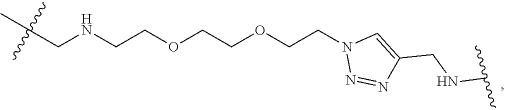 [SMILES: *CNCCOCCOCCn1cc(CN*)nn1] | (251) |
| 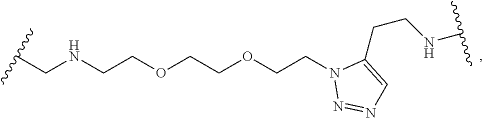 [SMILES: *CNCCOCCOCCn1nncc1CCN*] | (253) |
| 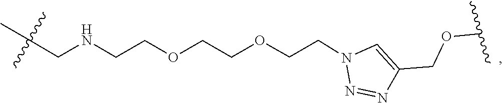 [SMILES: *CNCCOCCOCCn1cc(CO*)nn1] | (254) |
| 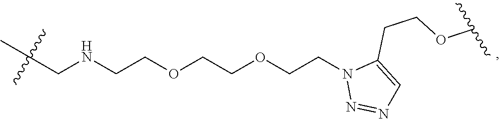 [SMILES: *CNCCOCCOCCn1nncc1CCO*] | (255) |
| 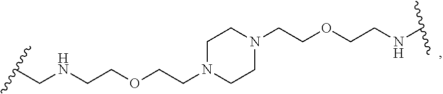 [SMILES: *CNCCOCCN1CCN(CCOCCN*)CC1] | (256) |
| 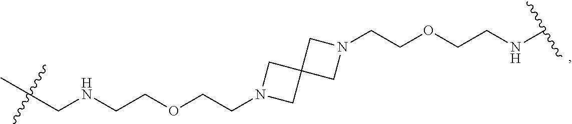 [SMILES: *CNCCOCCN1CC2(CN(CCOCCN*)C2)C1] | (257) |
| 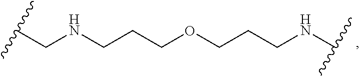 [SMILES: *CNCCCOCCCN*] | (258) |
| 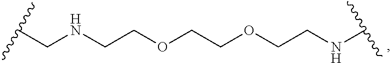 [SMILES: *CNCCOCCOCCN*] | (259) |
| 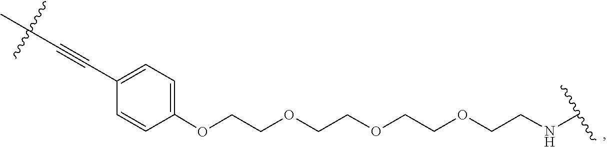 [SMILES: *C#Cc1ccc(OCCOCCOCCOCCN*)cc1] | (260) |
| 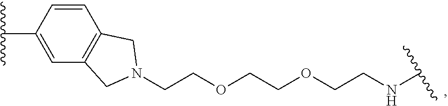 [SMILES: *NCCOCCOCCN1Cc2ccc(*)cc2C1] | (261) |
| 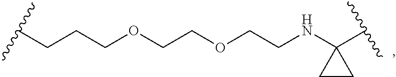 [SMILES: *CCCOCCOCCNC1(*)CC1] | (262) |
| 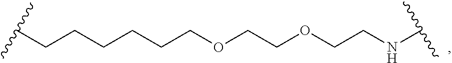 [SMILES: *CCCCCCOCCOCCN*] | (263) |
| 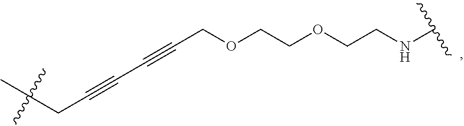 [SMILES: *CC#CC#CCOCCOCCN*] | (264) |
| 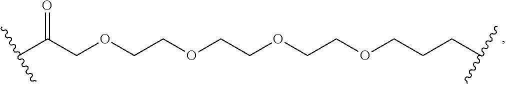 [SMILES: *CCCOCCOCCOCCOCC(*)=O] | (265) |
| 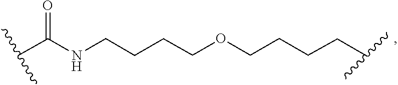 [SMILES: *CCCCOCCCCNC(*)=O] | (266) |
| 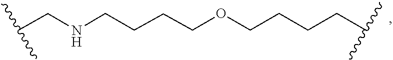 [SMILES: *CCCCOCCCCNC*] | (267) |
| 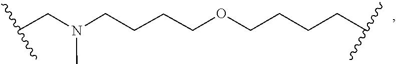 [SMILES: *CCCCOCCCCN(C)C*] | (268) |
| 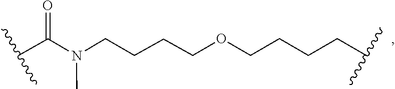 [SMILES: *CCCCOCCCCN(C)C(*)=O] | (269) |
| 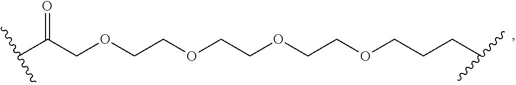 [SMILES: *CCCOCCOCCOCCOCC(*)=O] | (270) |
| 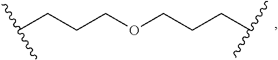 [SMILES: *CCCOCCC*] | (271) |
| 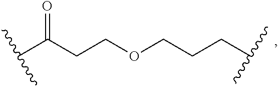 [SMILES: *CCCOCCC(*)=O] | (272) |
| 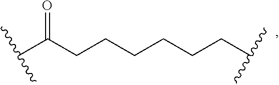 [SMILES: *CCCCCCC(*)=O] | (273) |
| 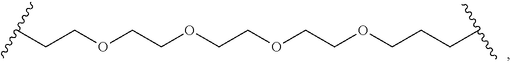 [SMILES: *CCCOCCOCCOCCOCC*] | (274) |
| 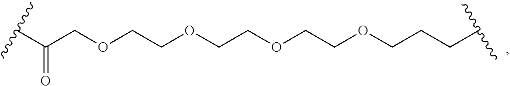 [SMILES: *CCCOCCOCCOCCOCC(*)=O] | (275) |
| 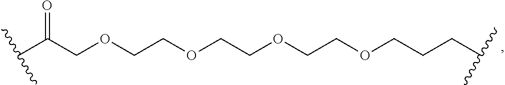 [SMILES: *CCCOCCOCCOCCOCC(*)=O] | (276) |
| 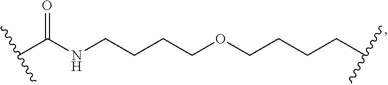 [SMILES: *CCCCOCCCCNC(*)=O] | (277) |
| 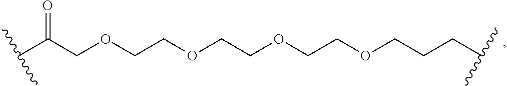 [SMILES: *CCCOCCOCCOCCOCC(*)=O] | (278) |
| 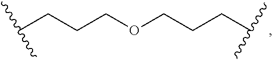 [SMILES: *CCCOCCC*] | (279) |
| 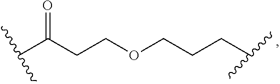 [SMILES: *CCCOCCC(*)=O] | (280) |
| 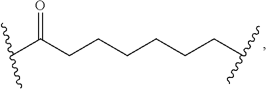 [SMILES: *CCCCCCC(*)=O] | (281) |
| 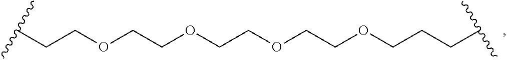 [SMILES: *CCCOCCOCCOCCOCC*] | (282) |
| 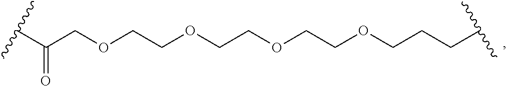 [SMILES: *CCCOCCOCCOCCOCC(*)=O] | (283) |
| 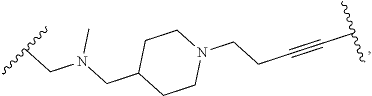 [SMILES: *C#CCCN1CCC(CN(C)C*)CC1] | (284) |
| 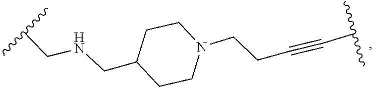 [SMILES: *C#CCCN1CCC(CNC*)CC1] | (285) |
| 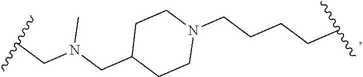 [SMILES: *CCCCN1CCC(CN(C)C*)CC1] | (286) |
| 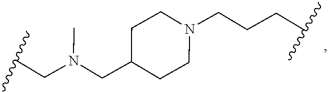 [SMILES: *CCCN1CCC(CN(C)C*)CC1] | (287) |
| 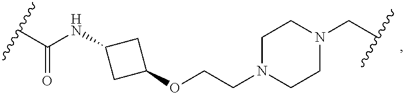 [SMILES: *CN1CCN(CCO[C@H]2C[C@H](NC(*)=O)C2)CC1] | (288) |
| 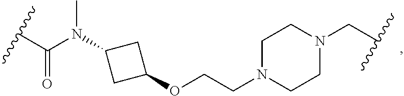 [SMILES: *CN1CCN(CCO[C@H]2C[C@H](N(C)C(*)=O)C2)CC1] | (289) |
| 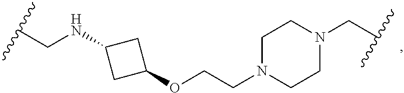 [SMILES: *CN[C@H]1C[C@H](OCCN2CCN(C*)CC2)C1] | (290) |
| 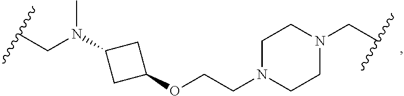 [SMILES: *CN1CCN(CCO[C@H]2C[C@H](N(C)C*)C2)CC1] | (291) |
| 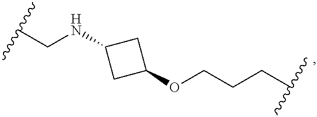 [SMILES: *CCCO[C@H]1C[C@H](NC*)C1] | (292) |
| 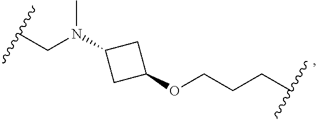 [SMILES: *CCCO[C@H]1C[C@H](N(C)C*)C1] | (293) |
| 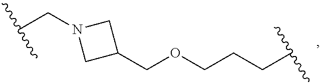 [SMILES: *CCCOCC1CN(C*)C1] | (294) |
| 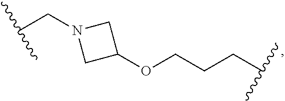 [SMILES: *CCCOC1CN(C*)C1] | (295) |
| 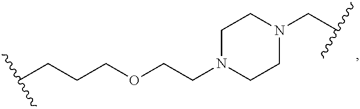 [SMILES: *CCCOCCN1CCN(C*)CC1] | (296) |
| 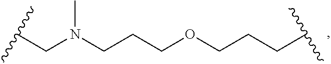 [SMILES: *CCCOCCCN(C)C*] | (297) |
| 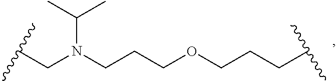 [SMILES: *CCCOCCCN(C*)C(C)C] | (298) |
| 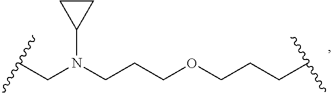 [SMILES: *CCCOCCCN(C*)C1CC1] | (299) |
| 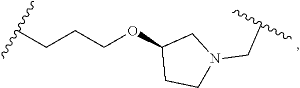 [SMILES: *CCCO[C@@H]1CCN(C*)C1] | (300) |
| 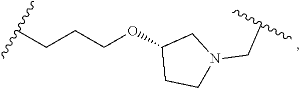 [SMILES: *CCCO[C@H]1CCN(C*)C1] | (301) |
| 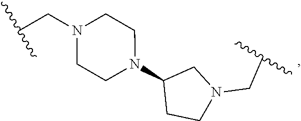 [SMILES: *CN1CCN([C@@H]2CCN(C*)C2)CC1] | (302) |
| 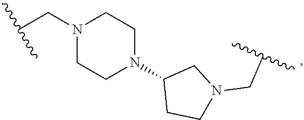 [SMILES: *CN1CCN([C@H]2CCN(C*)C2)CC1] | (303) |
| 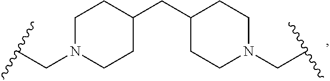 [SMILES: *CN1CCC(CC2CCN(C*)CC2)CC1] | (304) |
| 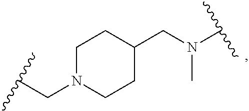 [SMILES: *CN1CCC(CN(*)C)CC1] | (305) |
| 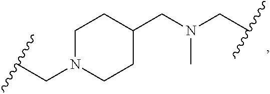 [SMILES: *CN(C)CC1CCN(C*)CC1] | (306) |
| 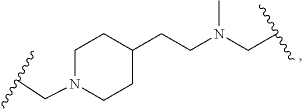 [SMILES: *CN(C)CCC1CCN(C*)CC1] | (307) |
| 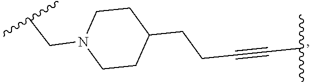 [SMILES: *C#CCCC1CCN(C*)CC1] | (308) |
| 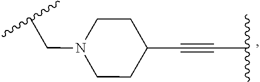 [SMILES: *C#CC1CCN(C*)CC1] | (309) |
| 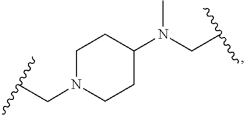 [SMILES: *CN1CCC(N(C)C*)CC1] | (310) |
| 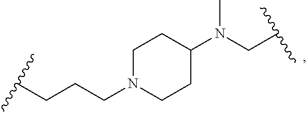 [SMILES: *CCCN1CCC(N(C)C*)CC1] | (311) |
| 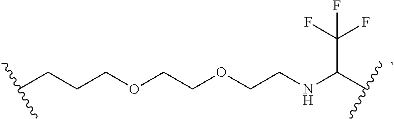 [SMILES: *CCCOCCOCCNC(*)C(F)(F)F] | (312) |
| 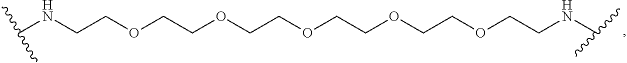 [SMILES: *NCCOCCOCCOCCOCCOCCN*] | (313) |
| 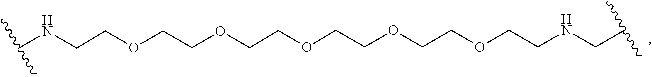 [SMILES: *CNCCOCCOCCOCCOCCOCCN*] | (314) |
| 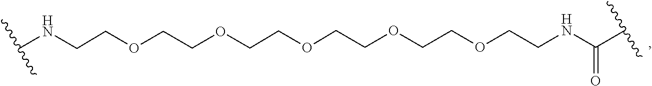 [SMILES: *NCCOCCOCCOCCOCCOCCNC(*)=O] | (315) |
| 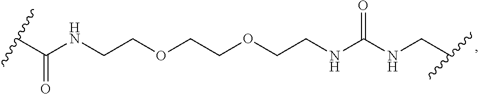 [SMILES: *CNC(=O)NCCOCCOCCNC(*)=O] | (316) |
| 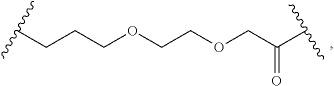 [SMILES: *CCCOCCOCC(*)=O] | (317) |
| 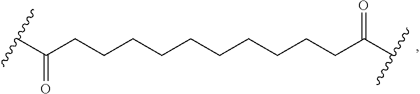 [SMILES: *C(=O)CCCCCCCCCCC(*)=O] | (318) |
| 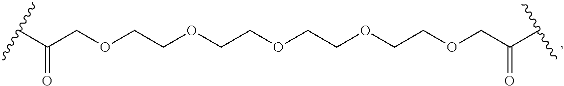 [SMILES: *C(=O)COCCOCCOCCOCCOCC(*)=O] | (319) |
| 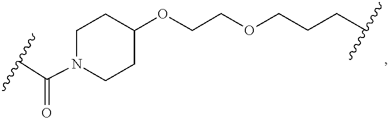 [SMILES: *CCCOCCOC1CCN(C(*)=O)CC1] | (320) |
| 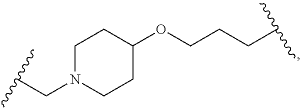 [SMILES: *CCCOC1CCN(C*)CC1] | (321) |
| 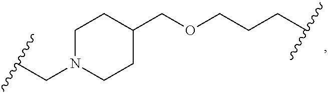 [SMILES: *CCCOCC1CCN(C*)CC1] | (322) |
| 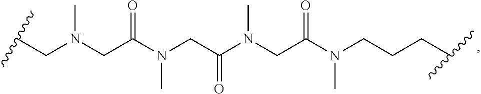 [SMILES: *CCCN(C)C(=O)CN(C)C(=O)CN(C)C(=O)CN(C)C*] | (323) |
| 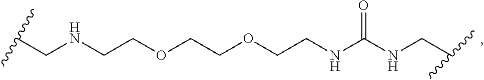 [SMILES: *CNCCOCCOCCNC(=O)NC*] | (324) |
| 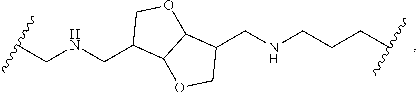 [SMILES: *CCCNCC1COC2C(CNC*)COC12] | (325) |
| 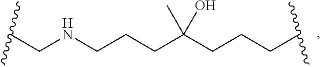 [SMILES: *CCCC(C)(O)CCCNC*] | (326) |
| 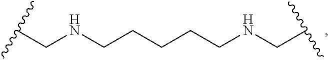 [SMILES: *CNCCCCCNC*] | (327) |
| 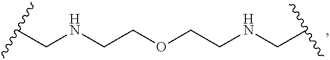 [SMILES: *CNCCOCCNC*] | (328) |
| 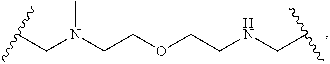 [SMILES: *CNCCOCCN(C)C*] | (329) |
| 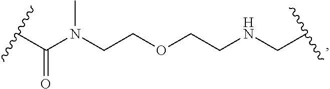 [SMILES: *CNCCOCCN(C)C(*)=O] | (330) |
| 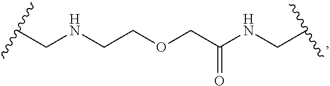 [SMILES: *CNCCOCC(=O)NC*] | (331) |
| 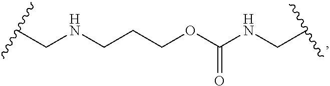 [SMILES: *CNCCCOC(=O)NC*] | (332) |
| 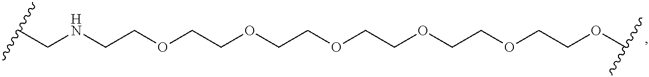 [SMILES: *CNCCOCCOCCOCCOCCOCCO*] | (333) |
| 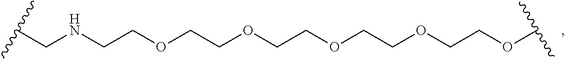 [SMILES: *CNCCOCCOCCOCCOCCO*] | (334) |
| 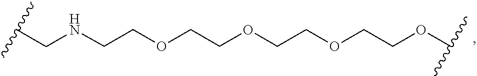 [SMILES: *CNCCOCCOCCOCCO*] | (335) |
| 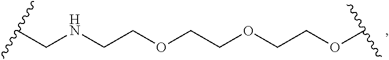 [SMILES: *CNCCOCCOCCO*] | (336) |
| 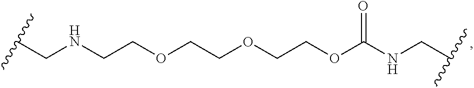 [SMILES: *CNCCOCCOCCOC(=O)NC*] | (337) |
| 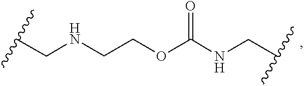 [SMILES: *CNCCOC(=O)NC*] | (338) |
| 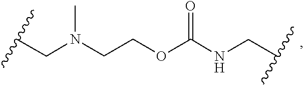 [SMILES: *CNC(=O)OCCN(C)C*] | (339) |
| 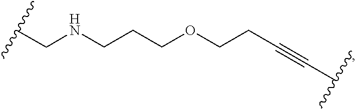 [SMILES: *C#CCCOCCCNC*] | (340) |
| 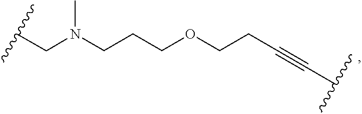 [SMILES: *C#CCCOCCCN(C)C*] | (341) |
| 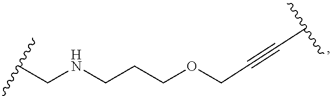 [SMILES: *C#CCOCCCNC*] | (342) |
| 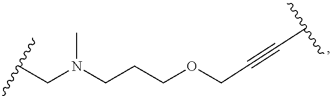 [SMILES: *C#CCOCCCN(C)C*] | (343) |
| 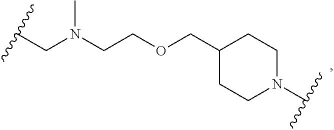 [SMILES: *CN(C)CCOCC1CCN(*)CC1] | (344) |
| 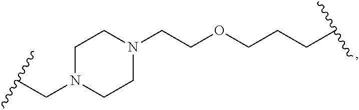 [SMILES: *CCCOCCN1CCN(C*)CC1] | (345) |
| 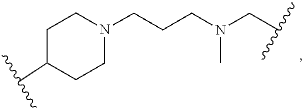 [SMILES: *CN(C)CCCN1CCC(*)CC1] | (346) |
| 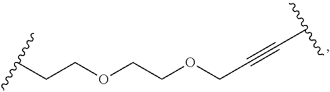 [SMILES: *C#CCOCCOCC*] | (347) |
| 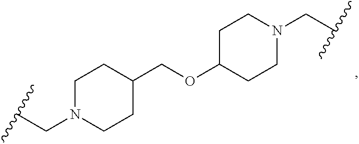 [SMILES: *CN1CCC(COC2CCN(C*)CC2)CC1] | (348) |
| 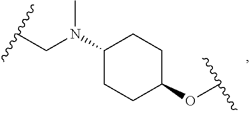 [SMILES: *CN(C)[C@H]1CC[C@H](O*)CC1] | (349) |
| 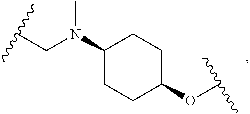 [SMILES: *CN(C)[C@H]1CC[C@@H](O*)CC1] | (350) |
| 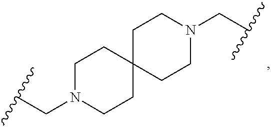 [SMILES: *CN1CCC2(CC1)CCN(C*)CC2] | (351) |
| 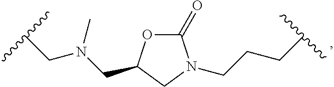 [SMILES: *CCCN1C[C@@H](CN(C)C*)OC1=O] | (352) |
| 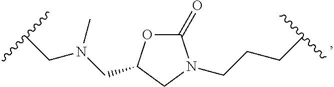 [SMILES: *CCCN1C[C@H](CN(C)C*)OC1=O] | (353) |
| 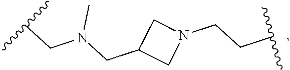 [SMILES: *CCN1CC(CN(C)C*)C1] | (354) |
| 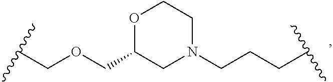 [SMILES: *CCCN1CCO[C@@H](COC*)C1] | (355) |
| 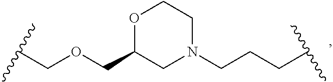 [SMILES: *CCCN1CCO[C@H](COC*)C1] | (356) |
| 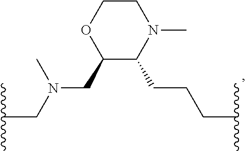 [SMILES: *CCC[C@@H]1[C@@H](CN(C)C*)OCCN1C] | (357) |
| 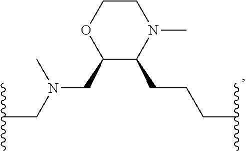 [SMILES: *CCC[C@H]1[C@@H](CN(C)C*)OCCN1C] | (358) |
| 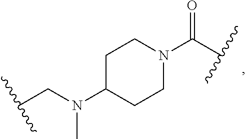 [SMILES: *CN(C)C1CCN(C(*)=O)CC1] | (359) |
| 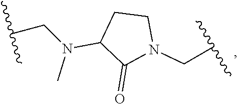 [SMILES: *CN1CCC(N(C)C*)C1=O] | (360) |
| 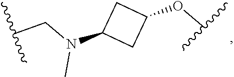 [SMILES: *CN(C)[C@H]1C[C@H](O*)C1] | (361) |
| 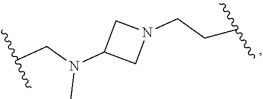 [SMILES: *CCN1CC(N(C)C*)C1] | (362) |
| 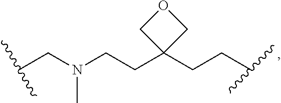 [SMILES: *CCC1(CCN(C)C*)COC1] | (363) |
| 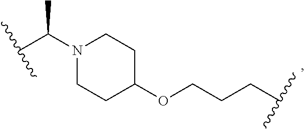 [SMILES: *CCCOC1CCN([C@@H](*)C)CC1] | (364) |
| 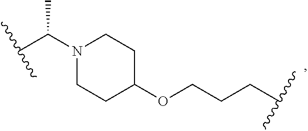 [SMILES: *CCCOC1CCN([C@H](*)C)CC1] | (365) |
| 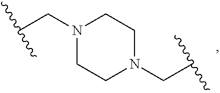 [SMILES: *CN1CCN(C*)CC1] | (366) |
| 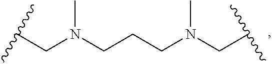 [SMILES: *CN(C)CCCN(C)C*] | (367) |
| 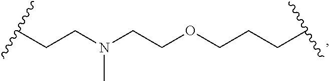 [SMILES: *CCCOCCN(C)CC*] | (368) |
| 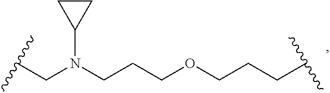 [SMILES: *CCCOCCCN(C*)C1CC1] | (369) |
| 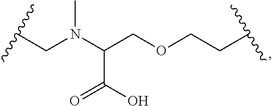 [SMILES: *CCOCC(C(=O)O)N(C)C*] | (370) |
| 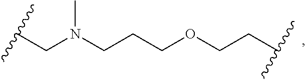 [SMILES: *CCOCCCN(C)C*] | (371) |
| 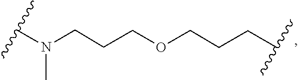 | (372) |
| 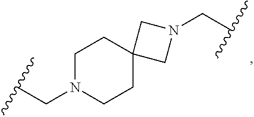 [SMILES: *CN1CCC2(CC1)CN(C*)C2] | (373) |
| 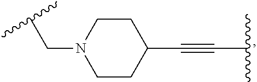 [SMILES: *C#CC1CCN(C*)CC1] | (374) |
| 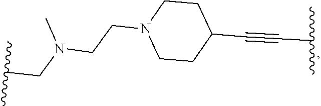 [SMILES: *C#CC1CCN(CCN(C)C*)CC1] | (375) |
| 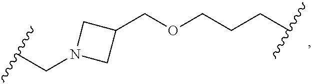 [SMILES: *CCCOCC1CN(C*)C1] | (376) |
| 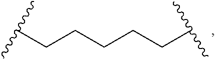 [SMILES: *CCCCC*] | (377) |
| 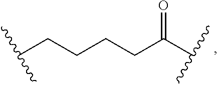 [SMILES: *CCCCC(*)=O] | (378) |
| 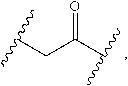 [SMILES: *CC(*)=O] | (379) |
| 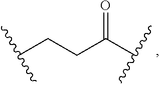 [SMILES: *CCC(*)=O] | (380) |
| 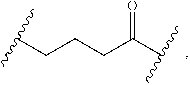 [SMILES: *CCCC(*)=O] | (381) |
| 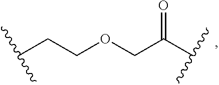 [SMILES: *CCOCC(*)=O] | (382) |
| 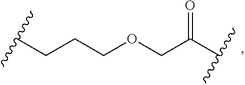 [SMILES: *CCCOCC(*)=O] | (383) |
| 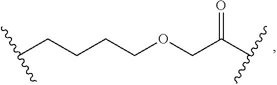 [SMILES: *CCCCOCC(*)=O] | (384) |
| 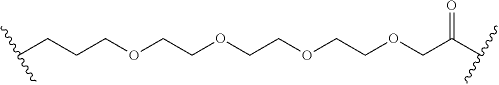 [SMILES: *CCCOCCOCCOCCOCC(*)=O] | (385) |
| 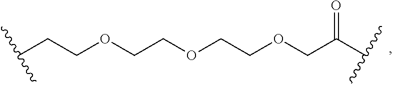 [SMILES: *CCOCCOCCOCC(*)=O] | (386) |
| 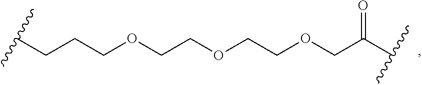 [SMILES: *CCCOCCOCCOCC(*)=O] | (387) |
| 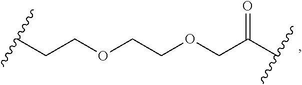 [SMILES: *CCOCCOCC(*)=O] | (388) |
| 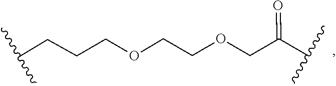 [SMILES: *CCCOCCOCC(*)=O] | (389) |
| 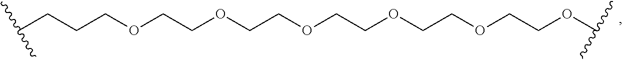 [SMILES: *CCCOCCOCCOCCOCCOCCO*] | (390) |
| 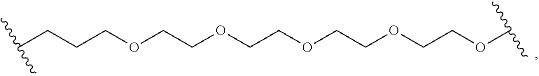 [SMILES: *CCCOCCOCCOCCOCCO*] | (391) |
| 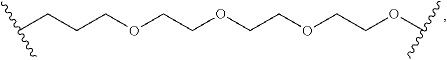 [SMILES: *CCCOCCOCCOCCO*] | (392) |
| 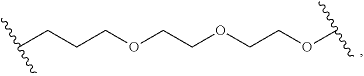 [SMILES: *CCCOCCOCCO*] | (393) |
| 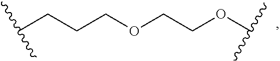 [SMILES: *CCCOCCO*] | (394) |
| 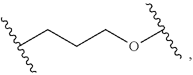 [SMILES: *CCCO*] | (395) |
| 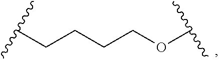 [SMILES: *CCCCO*] | (396) |
| 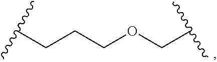 [SMILES: *CCCOC*] | (397) |
| 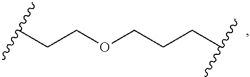 [SMILES: *CCCOCC*] | (398) |
| 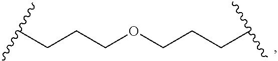 [SMILES: *CCCOCCC*] | (399) |
| 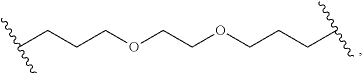 [SMILES: *CCCOCCOCCC*] | (400) |
| 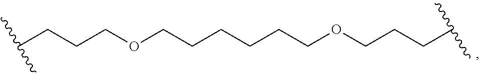 [SMILES: *CCCOCCCCCCOCCC*] | (401) |
| 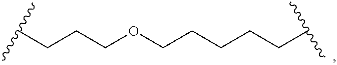 [SMILES: *CCCCCOCCC*] | (402) |
| 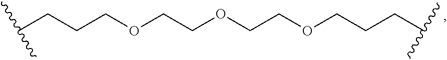 [SMILES: *CCCOCCOCCOCCC*] | (403) |
| 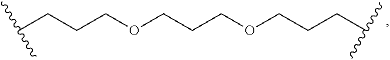 [SMILES: *CCCOCCCOCCC*] | (404) |
| 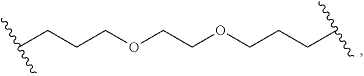 [SMILES: *CCCOCCOCCC*] | (405) |
| 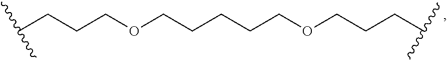 [SMILES: *CCCOCCCCCOCCC*] | (406) |
| 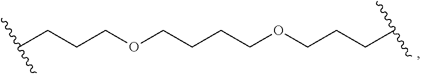 [SMILES: *CCCOCCCCOCCC*] | (407) |
| 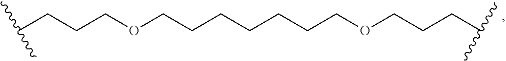 [SMILES: *CCCOCCCCCCCOCCC*] | (408) |
| 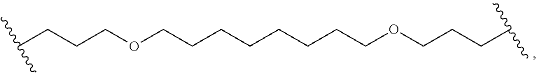 [SMILES: *CCCOCCCCCCCCOCCC*] | (409) |
| 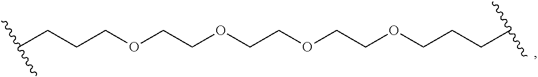 [SMILES: *CCCOCCOCCOCCOCCC*] | (410) |
| 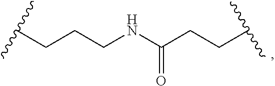 [SMILES: *CCCNC(=O)CC*] | (411) |
| 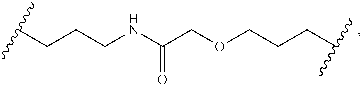 [SMILES: *CCCNC(=O)COCCC*] | (412) |
| 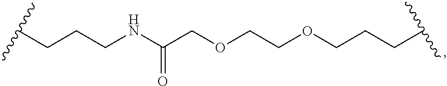 [SMILES: *CCCNC(=O)COCCOCCC*] | (413) |
| 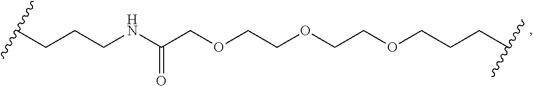 [SMILES: *CCCNC(=O)COCCOCCOCCC*] | (414) |
| 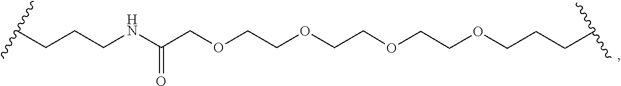 [SMILES: *CCCNC(=O)COCCOCCOCCOCCC*] | (415) |
| 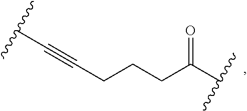 [SMILES: *C#CCCCC(*)=O] | (416) |
| 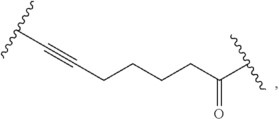 [SMILES: *C#CCCCCC(*)=O] | (417) |
| 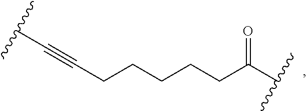 [SMILES: *C#CCCCCCC(*)=O] | (418) |
| 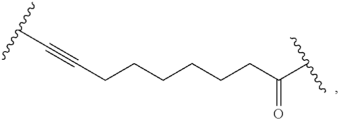 [SMILES: *C#CCCCCCCC(*)=O] | (419) |
| 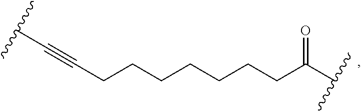 [SMILES: *C#CCCCCCCCC(*)=O] | (420) |
| 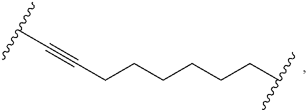 [SMILES: *C#CCCCCCC*] | (421) |
| 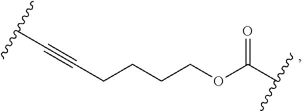 [SMILES: *C#CCCCCOC(*)=O] | (422) |
| 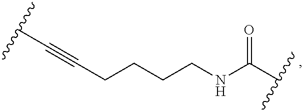 [SMILES: *C#CCCCCNC(*)=O] | (423) |
| 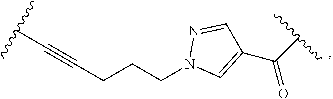 [SMILES: *C#CCCCn1cc(C(*)=O)cn1] | (424) |
| 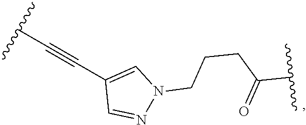 [SMILES: *C#Cc1cnn(CCCC(*)=O)c1] | (425) |
| 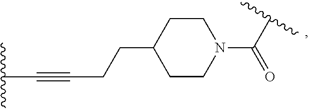 [SMILES: *C#CCCC1CCN(C(*)=O)CC1] | (426) |
| 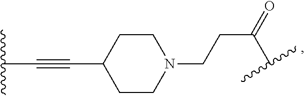 [SMILES: *C#CC1CCN(CCC(*)=O)CC1] | (427) |
| 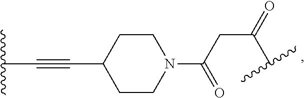 [SMILES: *C#CC1CCN(C(=O)CC(*)=O)CC1] | (428) |
| 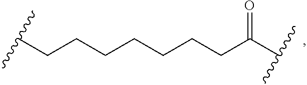 [SMILES: *CCCCCCCC(*)=O] | (429) |
| 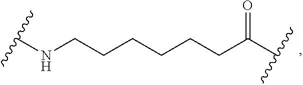 [SMILES: *NCCCCCCC(*)=O] | (430) |
| 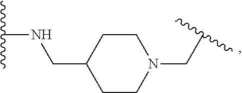 [SMILES: *CN1CCC(CN*)CC1] | (431) |
| 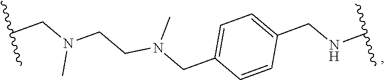 [SMILES: *CN(C)CCN(C)Cc1ccc(CN*)cc1] | (432) |
| 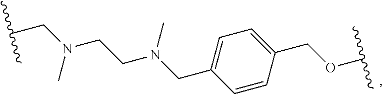 [SMILES: *CN(C)CCN(C)Cc1ccc(CO*)cc1] | (433) |
| 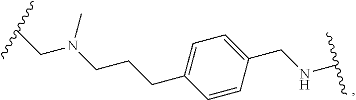 [SMILES: *CN(C)CCCc1ccc(CN*)cc1] | (434) |
| 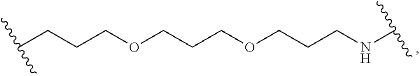 [SMILES: *CCCOCCCOCCCN*] | (435) |
| 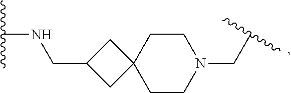 [SMILES: *CN1CCC2(CC1)CC(CN*)C2] | (436) |
| 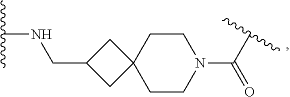 [SMILES: *NCC1CC2(CCN(C(*)=O)CC2)C1] | (437) |
| 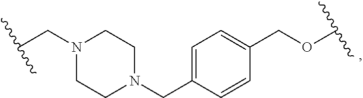 [SMILES: *CN1CCN(Cc2ccc(CO*)cc2)CC1] | (438) |
| 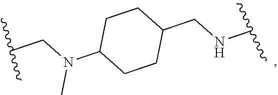 [SMILES: *CN(C)C1CCC(CN*)CC1] | (438) |
| 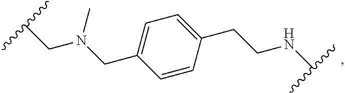 [SMILES: *CN(C)Cc1ccc(CCN*)cc1] | (439) |
| 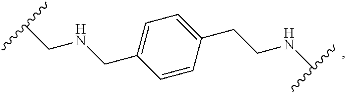 [SMILES: *CNCc1ccc(CCN*)cc1] | (440) |
| 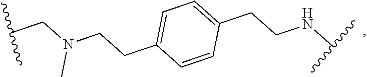 [SMILES: *CN(C)CCc1ccc(CCN*)cc1] | (441) |
| 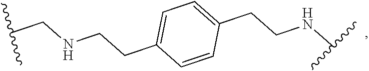 [SMILES: *CNCCc1ccc(CCN*)cc1] | (442) |
| 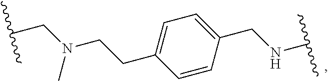 [SMILES: *CN(C)CCc1ccc(CN*)cc1] | (443) |
| 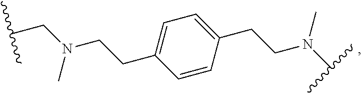 [SMILES: *CN(C)CCc1ccc(CCN(*)C)cc1] | (444) |
| 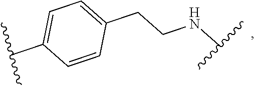 [SMILES: *NCCc1ccc(*)cc1] | (445) |
| 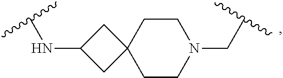 [SMILES: *CN1CCC2(CC1)CC(N*)C2] | (446) |
| 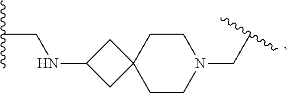 [SMILES: *CNC1CC2(CCN(C*)CC2)C1] | (447) |
| 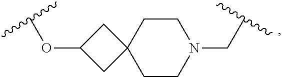 [SMILES: *CN1CCC2(CC1)CC(O*)C2] | (448) |
| 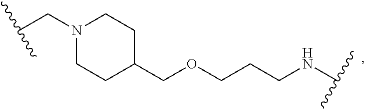 [SMILES: *CN1CCC(COCCCN*)CC1] | (449) |
| 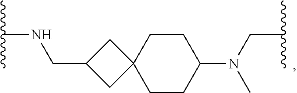 [SMILES: *CN(C)C1CCC2(CC1)CC(CN*)C2] | (450) |
| 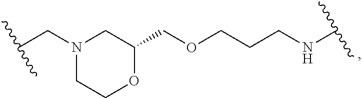 [SMILES: *CN1CCO[C@@H](COCCCN*)C1] | (451) |
| 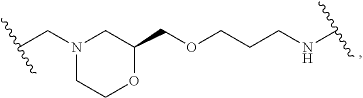 [SMILES: *CN1CCO[C@H](COCCCN*)C1] | (452) |
| 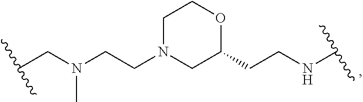 [SMILES: *CN(C)CCN1CCO[C@H](CCN*)C1] | (453) |
| 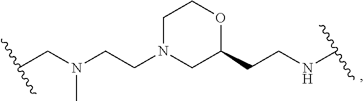 [SMILES: *CN(C)CCN1CCO[C@@H](CCN*)C1] | (454) |
| 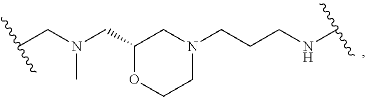 [SMILES: *CN(C)C[C@@H]1CN(CCCN*)CCO1] | (455) |
| 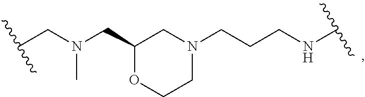 [SMILES: *CN(C)C[C@H]1CN(CCCN*)CCO1] | (456) |
| 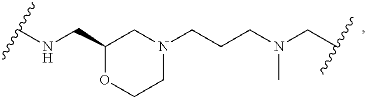 [SMILES: *CN(C)CCCN1CCO[C@@H](CN*)C1] | (457) |
| 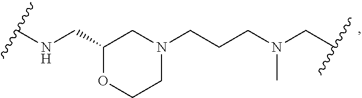 [SMILES: *CN(C)CCCN1CCO[C@H](CN*)C1] | (458) |
| 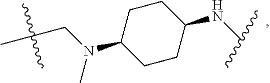 [SMILES: *CN(C)[C@H]1CC[C@@H](N*)CC1] | (459) |
| 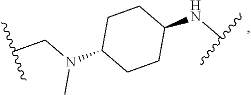 [SMILES: *CN(C)[C@H]1CC[C@H](N*)CC1] | (460) |
| 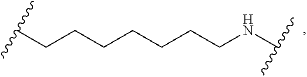 [SMILES: *CCCCCCCN*] | (461) |
| 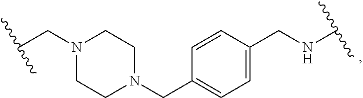 [SMILES: *CN1CCN(Cc2ccc(CN*)cc2)CC1] | (462) |
| 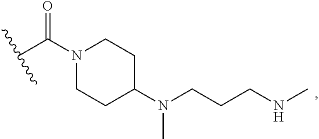 [SMILES: *C(=O)N1CCC(N(C)CCCNC)CC1] | (463) |
| 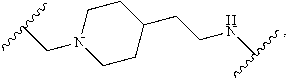 [SMILES: *CN1CCC(CCN*)CC1] | (464) |
| 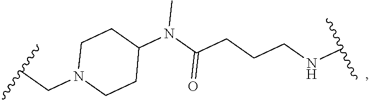 [SMILES: *CN1CCC(N(C)C(=O)CCCN*)CC1] | (465) |
| 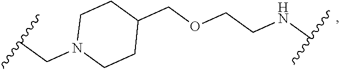 [SMILES: *CN1CCC(COCCN*)CC1] | (466) |
| 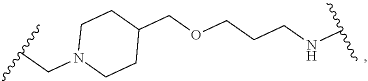 [SMILES: *CN1CCC(COCCCN*)CC1] | (467) |
| 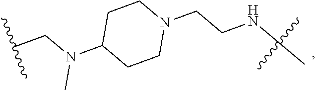 [SMILES: *CN(C)C1CCN(CCN*)CC1] | (468) |
| 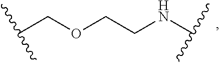 [SMILES: *COCCN*] | (469) |
| 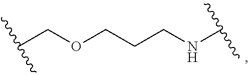 [SMILES: *COCCCN*] | (470) |
| 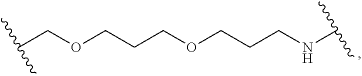 [SMILES: *COCCCOCCCN*] | (471) |
| 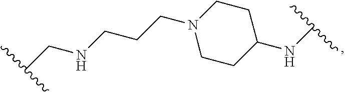 [SMILES: *CNCCCN1CCC(N*)CC1] | (472) |
| 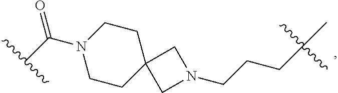 [SMILES: *CCCN1CC2(CCN(C(*)=O)CC2)C1] | (473) |
| 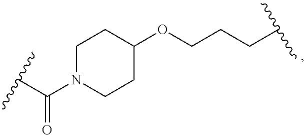 [SMILES: *CCCOC1CCN(C(*)=O)CC1] | (474) |
| 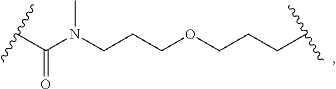 [SMILES: *CCCOCCCN(C)C(*)=O] | (475) |
| 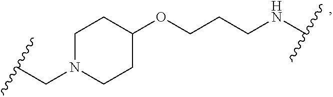 [SMILES: *CN1CCC(OCCCN*)CC1] | (475) |
| 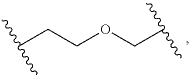 [SMILES: *CCOC*] | (476) |
| 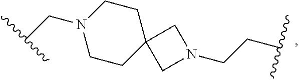 [SMILES: *CCN1CC2(CCN(C*)CC2)C1] | (477) |
| 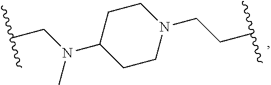 [SMILES: *CCN1CCC(N(C)C*)CC1] | (478) |
| 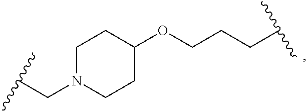 [SMILES: *CCCOC1CCN(C*)CC1] | (479) |
| 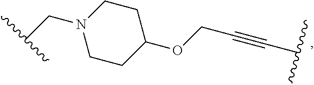 [SMILES: *C#CCOC1CCN(C*)CC1] | (480) |
| 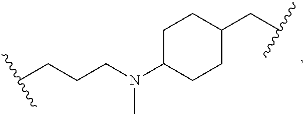 [SMILES: *CCCN(C)C1CCC(C*)CC1] | (481) |
| 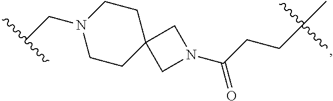 [SMILES: *CCC(=O)N1CC2(CCN(C*)CC2)C1] | (482) |
| 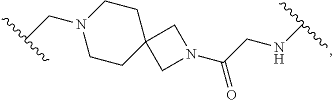 [SMILES: *CN1CCC2(CC1)CN(C(=O)CN*)C2] | (483) |
| 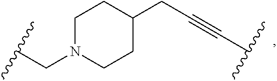 [SMILES: *C#CCC1CCN(C*)CC1] | (484) |
| 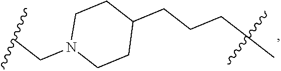 [SMILES: *CCCC1CCN(C*)CC1] | (485) |
| 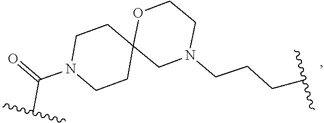 [SMILES: *CCCN1CCOC2(CCN(C(*)=O)CC2)C1] | (486) |
| 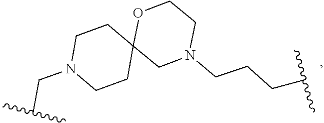 [SMILES: *CCCN1CCOC2(CCN(C*)CC2)C1] | (487) |
| 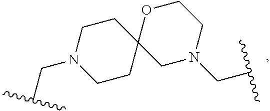 [SMILES: *CN1CCC2(CC1)CN(C*)CCO2] | (488) |
| 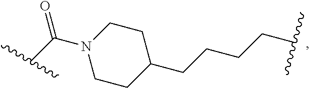 [SMILES: *CCCCC1CCN(C(*)=O)CC1] | (489) |
| 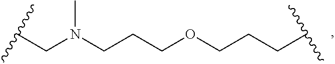 [SMILES: *CCCOCCCN(C)C*] | (490) |
| 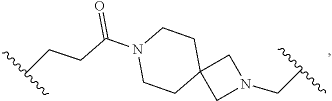 [SMILES: *CCC(=O)N1CCC2(CC1)CN(C*)C2] | (491) |
| 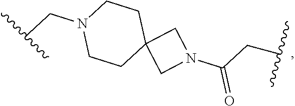 [SMILES: *CC(=O)N1CC2(CCN(C*)CC2)C1] | (492) |
| 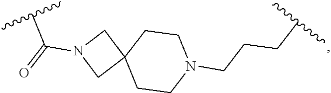 [SMILES: *CCCN1CCC2(CC1)CN(C(*)=O)C2] | (493) |
| 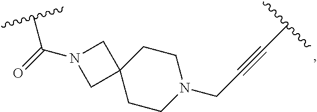 [SMILES: *C#CCN1CCC2(CC1)CN(C(*)=O)C2] | (494) |
| 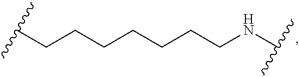 [SMILES: *CCCCCCCN*] | (495) |
| 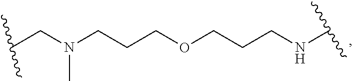 [SMILES: *CN(C)CCCOCCCN*] | (496) |
| 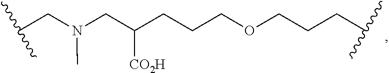 [SMILES: *CCCOCCCC(CN(C)C*)C(=O)O] | (497) |
| 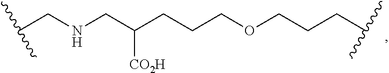 [SMILES: *CCCOCCCC(CNC*)C(=O)O] | (498) |
| 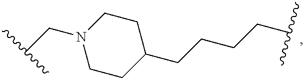 [SMILES: *CCCCC1CCN(C*)CC1] | (499) |
| 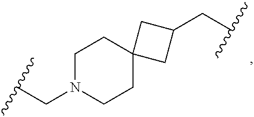 [SMILES: *CC1CC2(CCN(C*)CC2)C1] | (500) |
| 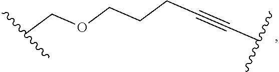 [SMILES: *C#CCCCOC*] | (501) |
| 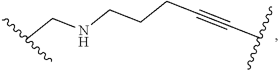 [SMILES: *C#CCCCNC*] | (502) |
| 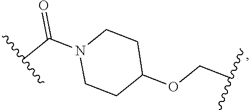 [SMILES: *COC1CCN(C(*)=O)CC1] | (503) |
| 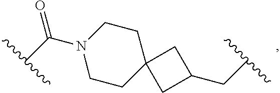 [SMILES: *CC1CC2(CCN(C(*)=O)CC2)C1] | (504) |
| 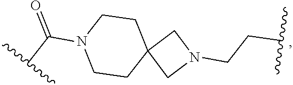 [SMILES: *CCN1CC2(CCN(C(*)=O)CC2)C1] | (505) |
| 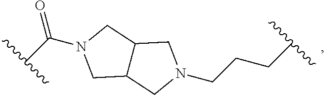 [SMILES: *CCCN1CC2CN(C(*)=O)CC2C1] | (506) |
| 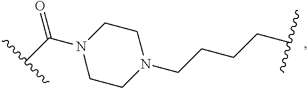 [SMILES: *CCCCN1CCN(C(*)=O)CC1] | (507) |
| 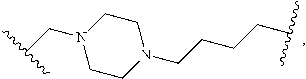 [SMILES: *CCCCN1CCN(C*)CC1] | (508) |
| 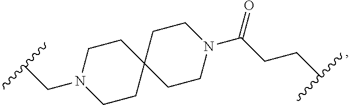 [SMILES: *CCC(=O)N1CCC2(CCN(C*)CC2)CC1] | (509) |
| 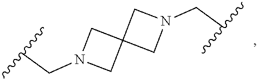 [SMILES: *CN1CC2(C1)CN(C*)C2] | (510) |
| 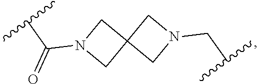 [SMILES: *CN1CC2(C1)CN(C(*)=O)C2] | (511) |
| 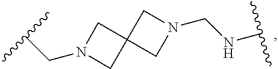 [SMILES: *CN1CC2(C1)CN(CN*)C2] | (512) |
| 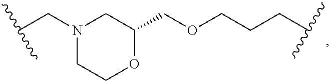 [SMILES: *CCCOC[C@H]1CN(C*)CCO1] | (513) |
| 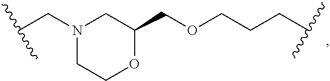 [SMILES: *CCCOC[C@@H]1CN(C*)CCO1] | (514) |
| 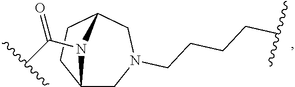 [SMILES: *CCCCN1C[C@H]2CC[C@@H](C1)N2C(*)=O] | (515) |
| 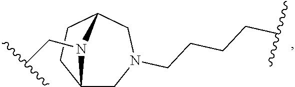 [SMILES: *CCCCN1C[C@H]2CC[C@@H](C1)N2C*] | (516) |
| 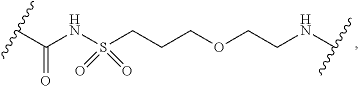 [SMILES: *NCCOCCCS(=O)(=O)NC(*)=O] | (517) |
| 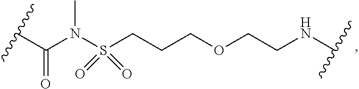 [SMILES: *NCCOCCCS(=O)(=O)N(C)C(*)=O] | (518) |
| 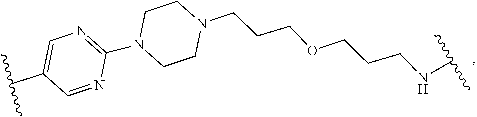 [SMILES: *NCCCOCCCN1CCN(c2ncc(*)cn2)CC1] | (519) |
| 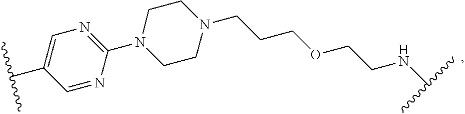 [SMILES: *NCCOCCCN1CCN(c2ncc(*)cn2)CC1] | (520) |
| 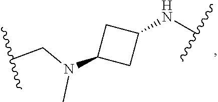 [SMILES: *CN(C)[C@H]1C[C@H](N*)C1] | (521) |
| 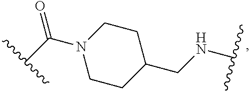 [SMILES: *NCC1CCN(C(*)=O)CC1] | (522) |
| 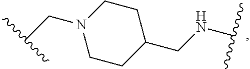 [SMILES: *CN1CCC(CN*)CC1] | (523) |
| 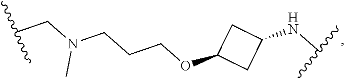 [SMILES: *CN(C)CCCO[C@H]1C[C@H](N*)C1] | (524) |
| 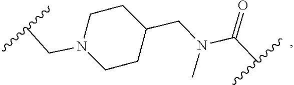 [SMILES: *CN1CCC(CN(C)C(*)=O)CC1] | (525) |
| 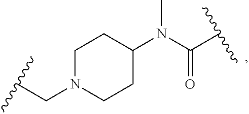 [SMILES: *CN1CCC(N(C)C(*)=O)CC1] | (526) |
| 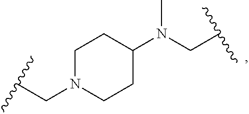 [SMILES: *CN1CCC(N(C)C*)CC1] | (527) |
| 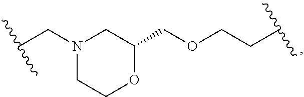 [SMILES: *CCOC[C@H]1CN(C*)CCO1] | (528) |
| 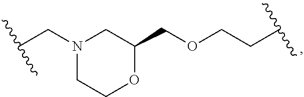 [SMILES: *CCOC[C@@H]1CN(C*)CCO1] | (529) |
| 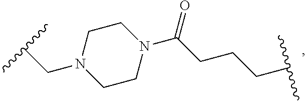 [SMILES: *CCCC(=O)N1CCN(C*)CC1] | (530) |
| 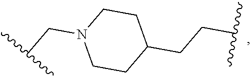 [SMILES: *CCC1CCN(C*)CC1] | (531) |
| 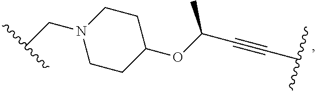 [SMILES: *C#C[C@H](C)OC1CCN(C*)CC1] | (532) |
| 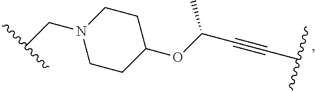 [SMILES: *C#C[C@@H](C)OC1CCN(C*)CC1] | (533) |
| 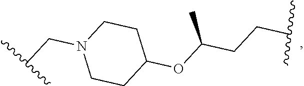 [SMILES: *CC[C@H](C)OC1CCN(C*)CC1] | (534) |
| 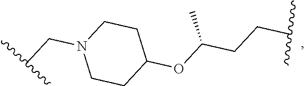 [SMILES: *CC[C@@H](C)OC1CCN(C*)CC1] | (535) |
| 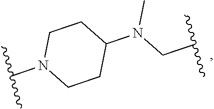 [SMILES: *CN(C)C1CCN(*)CC1] | (536) |
| 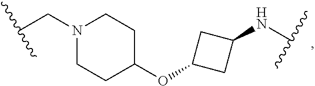 [SMILES: *CN1CCC(O[C@H]2C[C@H](N*)C2)CC1] | (537) |
| 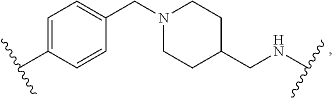 [SMILES: *NCC1CCN(Cc2ccc(*)cc2)CC1] | (538) |
| 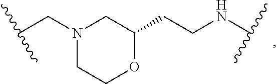 [SMILES: *CN1CCO[C@@H](CCN*)C1] | (539) |
| 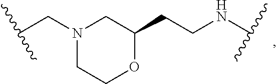 [SMILES: *CN1CCO[C@H](CCN*)C1] | (540) |
| 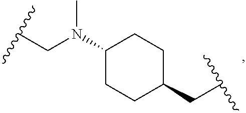 [SMILES: *CN(C)[C@H]1CC[C@H](C*)CC1] | (541) |
| 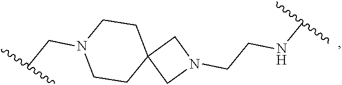 [SMILES: *CN1CCC2(CC1)CN(CCN*)C2] | (542) |
| 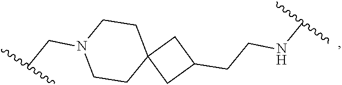 [SMILES: *CN1CCC2(CC1)CC(CCN*)C2] | (543) |
| 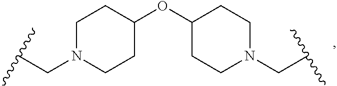 [SMILES: *CN1CCC(OC2CCN(C*)CC2)CC1] | (544) |
| 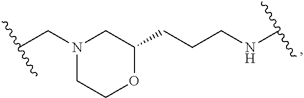 [SMILES: *CN1CCO[C@@H](CCCN*)C1] | (545) |
| 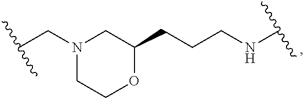 [SMILES: *CN1CCO[C@H](CCCN*)C1] | (546) |
| 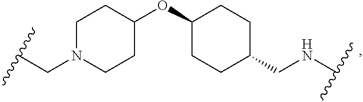 [SMILES: *CN1CCC(O[C@H]2CC[C@H](CN*)CC2)CC1] | (547) |
| 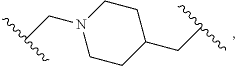 [SMILES: *CC1CCN(C*)CC1] | (548) |
| 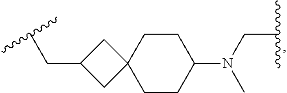 [SMILES: *CC1CC2(CCC(N(C)C*)CC2)C1] | (549) |
| 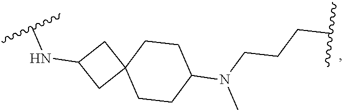 [SMILES: *CCCN(C)C1CCC2(CC1)CC(N*)C2] | (550) |
| 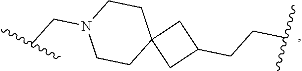 [SMILES: *CCC1CC2(CCN(C*)CC2)C1] | (551) |
| 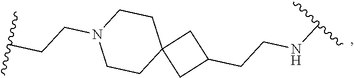 [SMILES: *CCN1CCC2(CC1)CC(CCN*)C2] | (552) |
| 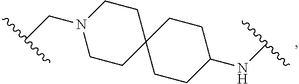 [SMILES: *CN1CCC2(CCC(N*)CC2)CC1] | (553) |
| 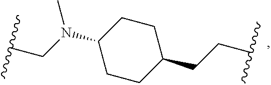 [SMILES: *CC[C@H]1CC[C@H](N(C)C*)CC1] | (554) |
| 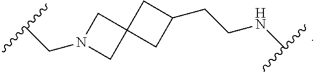 [SMILES: *CN1CC2(CC(CCN*)C2)C1] | (555) |
| 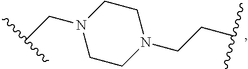 [SMILES: *CCN1CCN(C*)CC1] | (556) |
| 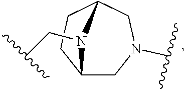 [SMILES: *CN1[C@@H]2CC[C@H]1CN(*)C2] | (557) |
| 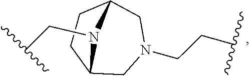 [SMILES: *CCN1C[C@H]2CC[C@@H](C1)N2C*] | (558) |
| 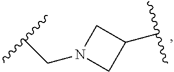 [SMILES: *CN1CC(*)C1] | (559) |
| 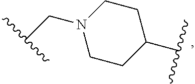 [SMILES: *CN1CCC(*)CC1] | (560) |
| 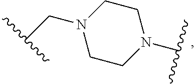 [SMILES: *CN1CCN(*)CC1] | (561) |
| 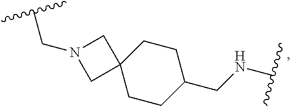 [SMILES: *CN1CC2(CCC(CN*)CC2)C1] | (562) |
| 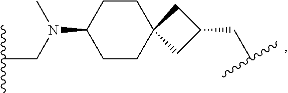 | (563) |
| 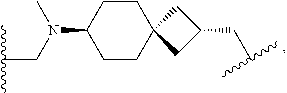 | (564) |
| 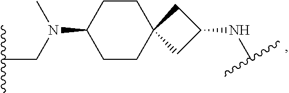 | (565) |
| 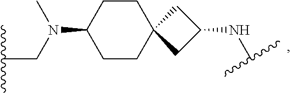 | (566) |
| 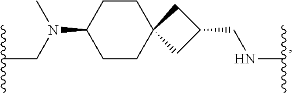 | (567) |
| 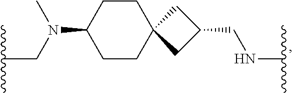 | (568) |
| 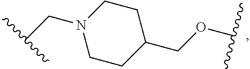 [SMILES: *CN1CCC(CO*)CC1] | (569) |
| 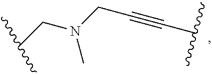 [SMILES: *C#CCN(C)C*] | (570) |
| 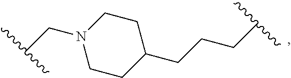 [SMILES: *CCCC1CCN(C*)CC1] | (571) |
| 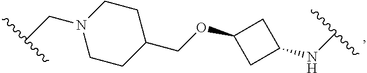 [SMILES: *CN1CCC(CO[C@H]2C[C@H](N*)C2)CC1] | (572) |
| 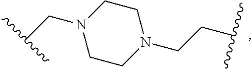 [SMILES: *CCN1CCN(C*)CC1] | (573) |
| 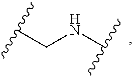 [SMILES: *CN*] | (574) |
| 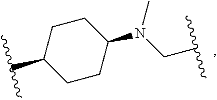 [SMILES: *CN(C)[C@H]1CC[C@@H](*)CC1] | (575) |
| 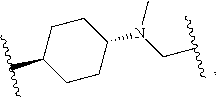 [SMILES: *CN(C)[C@H]1CC[C@H](*)CC1] | (576) |
| 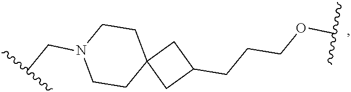 [SMILES: *CN1CCC2(CC1)CC(CCCO*)C2] | (577) |
| 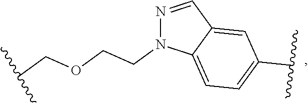 [SMILES: *COCCn1ncc2cc(*)ccc21] | (578) |
| 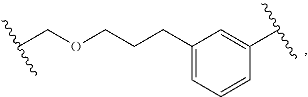 [SMILES: *COCCCc1cccc(*)c1] | (579) |
| 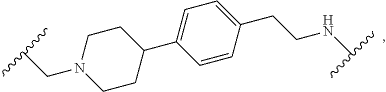 [SMILES: *CN1CCC(c2ccc(CCN*)cc2)CC1] | (580) |
| 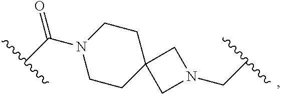 [SMILES: *CN1CC2(CCN(C(*)=O)CC2)C1] | (581) |
| 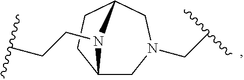 [SMILES: *CCN1[C@@H]2CC[C@H]1CN(C*)C2] | (582) |
| 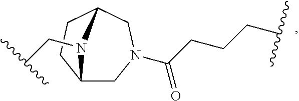 [SMILES: *CCCC(=O)N1C[C@H]2CC[C@@H](C1)N2C*] | (583) |
| 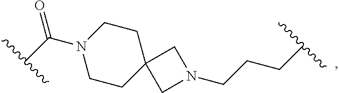 [SMILES: *CCCN1CC2(CCN(C(*)=O)CC2)C1] | (584) |
| 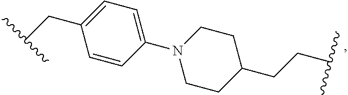 [SMILES: *CCC1CCN(c2ccc(C*)cc2)CC1] | (585) |
| 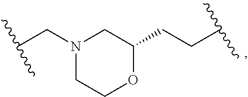 [SMILES: *CC[C@H]1CN(C*)CCO1] | (586) |
| 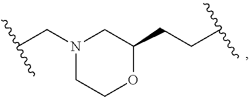 [SMILES: *CC[C@@H]1CN(C*)CCO1] | (587) |
| 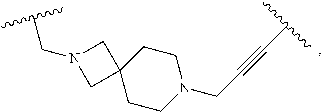 [SMILES: *C#CCN1CCC2(CC1)CN(C*)C2] | (588) |
| 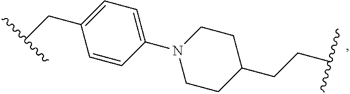 [SMILES: *CCC1CCN(c2ccc(C*)cc2)CC1] | (589) |
| 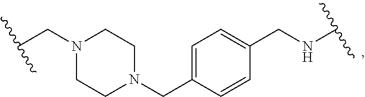 [SMILES: *CN1CCN(Cc2ccc(CN*)cc2)CC1] | (590) |
| 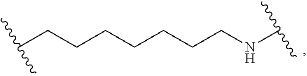 [SMILES: *CCCCCCCN*] | (591) |
| 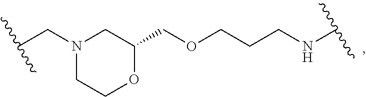 [SMILES: *CN1CCO[C@@H](COCCCN*)C1] | (592) |
| 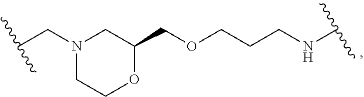 [SMILES: *CN1CCO[C@H](COCCCN*)C1] | (593) |
| 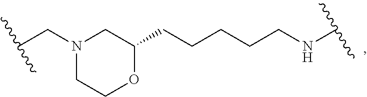 [SMILES: *CN1CCO[C@@H](CCCCCN*)C1] | (594) |
| 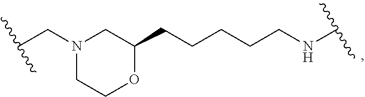 [SMILES: *CN1CCO[C@H](CCCCCN*)C1] | (595) |
| 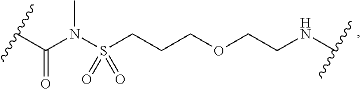 [SMILES: *NCCOCCCS(=O)(=O)N(C)C(*)=O] | (596) |
| 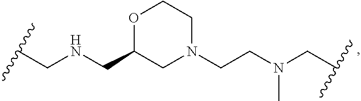 [SMILES: *CNC[C@@H]1CN(CCN(C)C*)CCO1] | (597) |
| 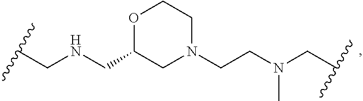 [SMILES: *CNC[C@H]1CN(CCN(C)C*)CCO1] | (598) |
| 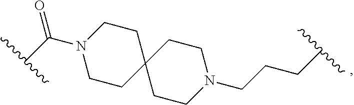 [SMILES: *CCCN1CCC2(CC1)CCN(C(*)=O)CC2] | (599) |
| 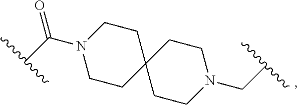 [SMILES: *CN1CCC2(CC1)CCN(C(*)=O)CC2] | (600) |
| 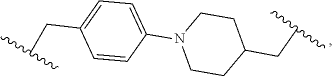 [SMILES: *Cc1ccc(N2CCC(C*)CC2)cc1] | (601) |
| 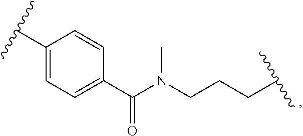 [SMILES: *CCCN(C)C(=O)c1ccc(*)cc1] | (602) |
| 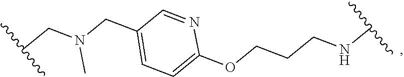 [SMILES: *CN(C)Cc1ccc(OCCCN*)nc1] | (603) |
| 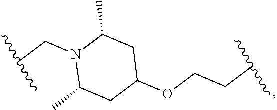 [SMILES: *CCOC1C[C@@H](C)N(C*)[C@@H](C)C1] | (604) |
| 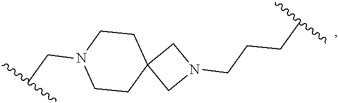 [SMILES: *CCCN1CC2(CCN(C*)CC2)C1] | (605) |
| 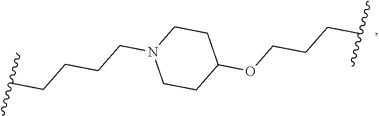 [SMILES: *CCCCN1CCC(OCCC*)CC1] | (606) |
| 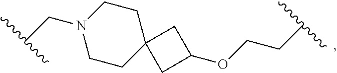 [SMILES: *CCOC1CC2(CCN(C*)CC2)C1] | (607) |
| 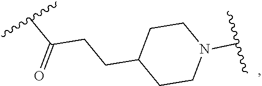 [SMILES: *C(=O)CCC1CCN(*)CC1] | (608) |
| 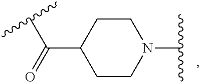 [SMILES: *C(=O)C1CCN(*)CC1] | (609) |
| 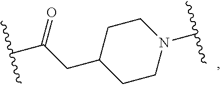 [SMILES: *C(=O)CC1CCN(*)CC1] | (610) |
| 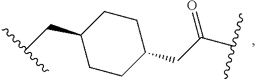 [SMILES: *C[C@H]1CC[C@H](CC(*)=O)CC1] | (611) |
| 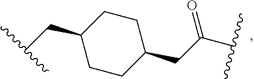 [SMILES: *C[C@H]1CC[C@@H](CC(*)=O)CC1] | (612) |
| 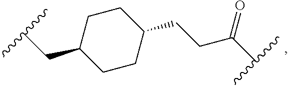 [SMILES: *C[C@H]1CC[C@H](CCC(*)=O)CC1] | (613) |
| 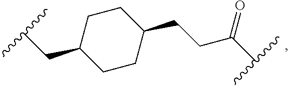 [SMILES: *C[C@H]1CC[C@@H](CCC(*)=O)CC1] | (614) |
| 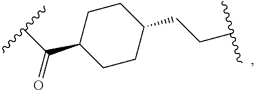 [SMILES: *CC[C@H]1CC[C@H](C(*)=O)CC1] | (615) |
| 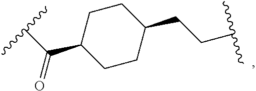 [SMILES: *CC[C@H]1CC[C@@H](C(*)=O)CC1] | (616) |
| 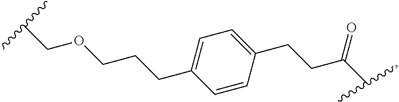 [SMILES: *COCCCc1ccc(CCC(*)=O)cc1] | (617) |
| 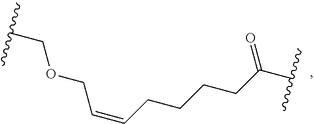 [SMILES: *COC/C=C\CCCCC(*)=O] | (618) |
| 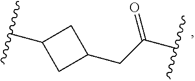 [SMILES: *C(=O)CC1CC(*)C1] | (619) |
| 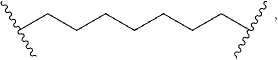 [SMILES: *CCCCCCC*] | (620) |
| 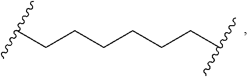 [SMILES: *CCCCCC*] | (621) |
| 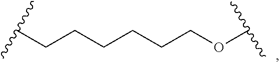 [SMILES: *CCCCCCO*] | (622) |
| 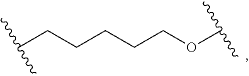 [SMILES: *CCCCCO*] | (623) |
| 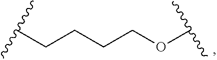 [SMILES: *CCCCO*] | (624) |
| 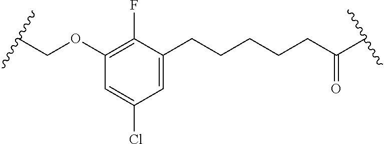 [SMILES: *COc1cc(Cl)cc(CCCCCC(*)=O)c1F] | (625) |
| 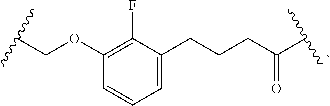 [SMILES: *COc1cccc(CCCC(*)=O)c1F] | (626) |
| 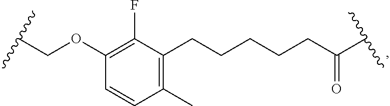 [SMILES: *COc1ccc(C)c(CCCCCC(*)=O)c1F] | (627) |
| 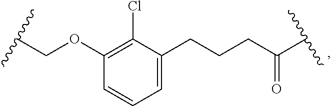 [SMILES: *COc1cccc(CCCC(*)=O)c1Cl] | (628) |
| 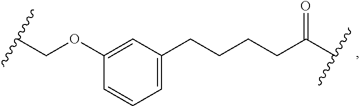 [SMILES: *COc1cccc(CCCCC(*)=O)c1] | (629) |
| 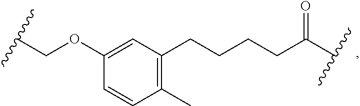 [SMILES: *COc1ccc(C)c(CCCCC(*)=O)c1] | (630) |
| 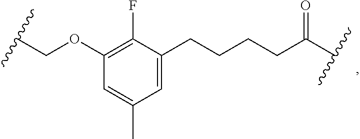 [SMILES: *COc1cc(C)cc(CCCCC(*)=O)c1F] | (631) |
| 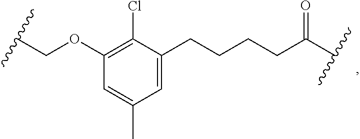 [SMILES: *COc1cc(C)cc(CCCCC(*)=O)c1Cl] | (632) |
| 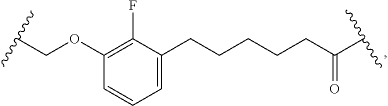 [SMILES: *COc1cccc(CCCCCC(*)=O)c1F] | (633) |
| 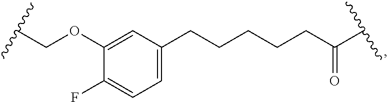 [SMILES: *COc1cc(CCCCCC(*)=O)ccc1F] | (634) |
| 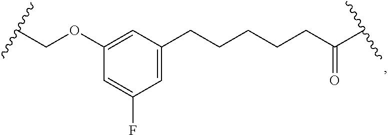 [SMILES: *COc1cc(F)cc(CCCCCC(*)=O)c1] | (635) |
| 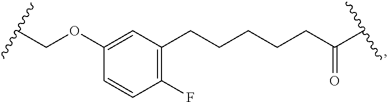 [SMILES: *COc1ccc(F)c(CCCCCC(*)=O)c1] | (636) |
| 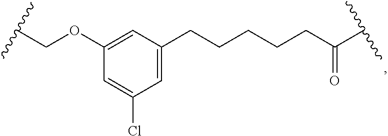 [SMILES: *COc1cc(Cl)cc(CCCCCC(*)=O)c1] | (637) |
| 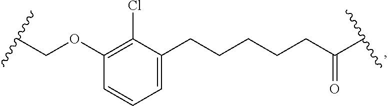 [SMILES: *COc1cccc(CCCCCC(*)=O)c1Cl] | (638) |
| 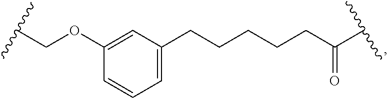 [SMILES: *COc1cccc(CCCCCC(*)=O)c1] | (639) |
| 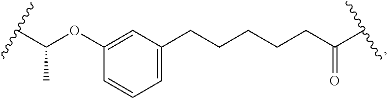 [SMILES: *C(=O)CCCCCc1cccc(O[C@@H](*)C)c1] | (640) |
| 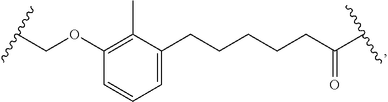 [SMILES: *COc1cccc(CCCCCC(*)=O)c1C] | (641) |
| 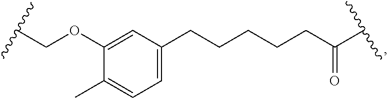 [SMILES: *COc1cc(CCCCCC(*)=O)ccc1C] | (642) |
| 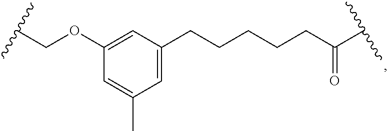 [SMILES: *COc1cc(C)cc(CCCCCC(*)=O)c1] | (643) |
| 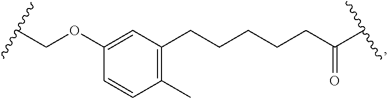 [SMILES: *COc1ccc(C)c(CCCCCC(*)=O)c1] | (644) |
| 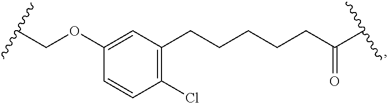 [SMILES: *COc1ccc(Cl)c(CCCCCC(*)=O)c1] | (645) |
| 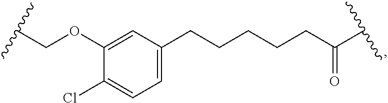 [SMILES: *COc1cc(CCCCCC(*)=O)ccc1Cl] | (646) |
| 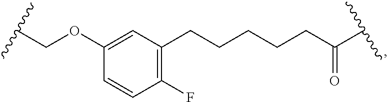 [SMILES: *COc1ccc(F)c(CCCCCC(*)=O)c1] | (647) |
| 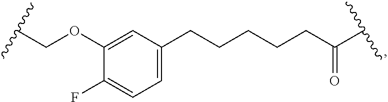 [SMILES: *COc1cc(CCCCCC(*)=O)ccc1F] | (648) |
| 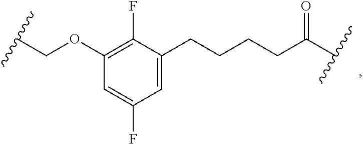 [SMILES: *COc1cc(F)cc(CCCCC(*)=O)c1F] | (649) |
| 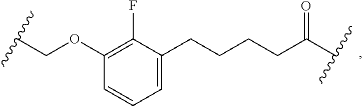 [SMILES: *COc1cccc(CCCCC(*)=O)c1F] | (650) |
| 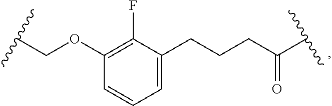 [SMILES: *COc1cccc(CCCC(*)=O)c1F] | (651) |
| 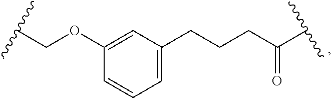 [SMILES: *COc1cccc(CCCC(*)=O)c1] | (652) |
| 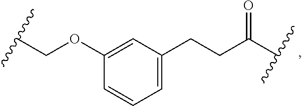 [SMILES: *COc1cccc(CCC(*)=O)c1] | (653) |
| 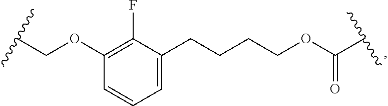 [SMILES: *COc1cccc(CCCCOC(*)=O)c1F] | (654) |
| 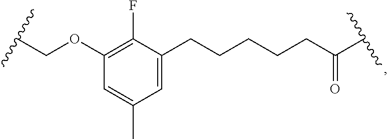 [SMILES: *COc1cc(C)cc(CCCCCC(*)=O)c1F] | (655) |
| 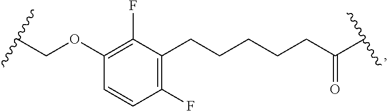 [SMILES: *COc1ccc(F)c(CCCCCC(*)=O)c1F] | (656) |
| 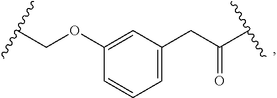 [SMILES: *COc1cccc(CC(*)=O)c1] | (657) |
| 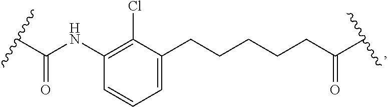 [SMILES: *C(=O)CCCCCc1cccc(NC(*)=O)c1Cl] | (658) |
| 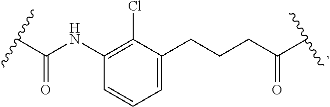 [SMILES: *C(=O)CCCc1cccc(NC(*)=O)c1Cl] | (659) |
| 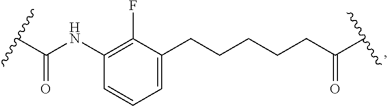 [SMILES: *C(=O)CCCCCc1cccc(NC(*)=O)c1F] | (660) |
| 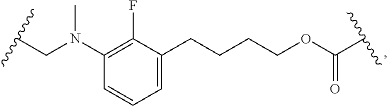 [SMILES: *CN(C)c1cccc(CCCCOC(*)=O)c1F] | (661) |
| 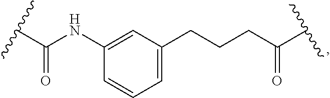 [SMILES: *C(=O)CCCc1cccc(NC(*)=O)c1] | (662) |
| 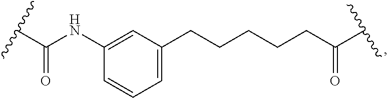 [SMILES: *C(=O)CCCCCc1cccc(NC(*)=O)c1] | (663) |
| 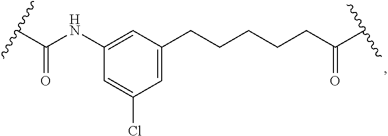 [SMILES: *C(=O)CCCCCc1cc(Cl)cc(NC(*)=O)c1] | (664) |
| 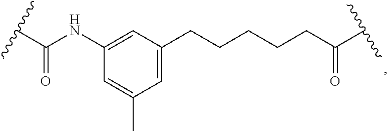 [SMILES: *C(=O)CCCCCc1cc(C)cc(NC(*)=O)c1] | (665) |
| 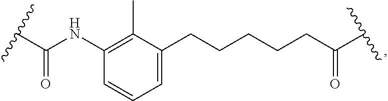 [SMILES: *C(=O)CCCCCc1cccc(NC(*)=O)c1C] | (666) |
| 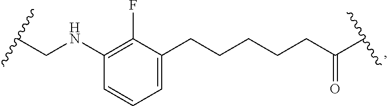 [SMILES: *CNc1cccc(CCCCCC(*)=O)c1F] | (667) |
| 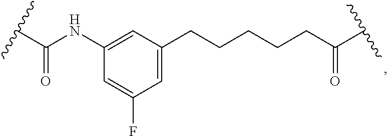 [SMILES: *C(=O)CCCCCc1cc(F)cc(NC(*)=O)c1] | (668) |
| 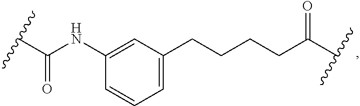 [SMILES: *C(=O)CCCCc1cccc(NC(*)=O)c1] | (669) |
| 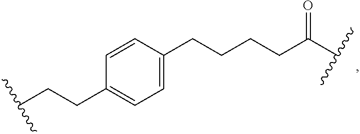 [SMILES: *CCc1ccc(CCCCC(*)=O)cc1] | (670) |
| 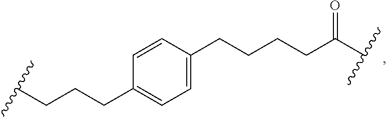 [SMILES: *CCCc1ccc(CCCCC(*)=O)cc1] | (671) |
| 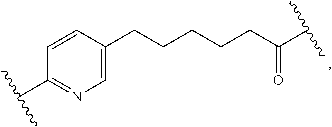 [SMILES: *C(=O)CCCCCc1ccc(*)nc1] | (672) |
| 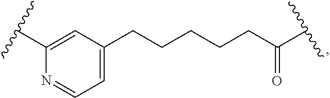 [SMILES: *C(=O)CCCCCc1ccnc(*)c1] | (673) |
| 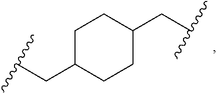 [SMILES: *CC1CCC(C*)CC1] | (674) |
| 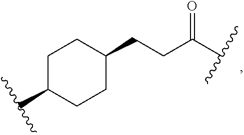 [SMILES: *C(=O)CC[C@H]1CC[C@@H](*)CC1] | (675) |
| 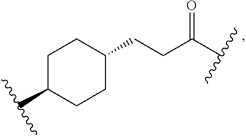 [SMILES: *C(=O)CC[C@H]1CC[C@H](*)CC1] | (676) |
| 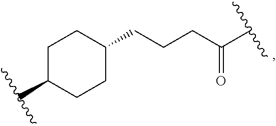 [SMILES: *C(=O)CCC[C@H]1CC[C@H](*)CC1] | (677) |
| 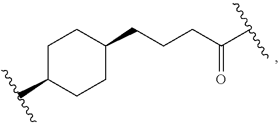 [SMILES: *C(=O)CCC[C@H]1CC[C@@H](*)CC1] | (678) |
| 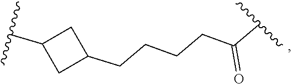 [SMILES: *C(=O)CCCCC1CC(*)C1] | (679) |
| 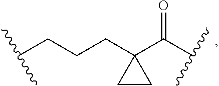 [SMILES: *CCCC1(C(*)=O)CC1] | (680) |
| or any one of the linkers described in the Linker section above | |

In certain embodiments, the present invention provides a compound of Formula I-a″″-1, as a compound of Formula I-bb:

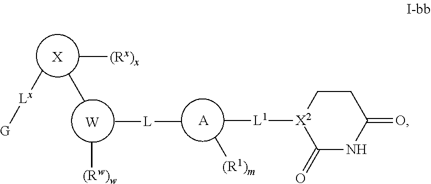

or a pharmaceutically acceptable salt thereof, wherein L, L^{X}, L¹, Ring A, Ring W, Ring X, G, R^{w}, R^{x}, R¹, X², m, w, and x are as defined above and described herein both individually and in combination.

In some embodiments, a provided compound is a compound of Formula I-bb:

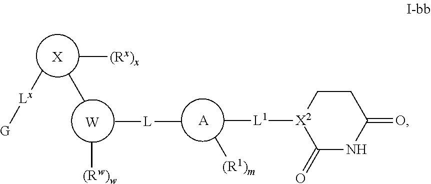

or a pharmaceutically acceptable salt thereof, wherein each of R^{W}, w, R^{X}, x, R^{y}, y, and L is as defined and described above and herein, and wherein:
X² is N or CH;
L¹ is a covalent bond, —C(O)—, —NR—, —O—, —S—, —S(O)₂, —NRC(O)—, or —C(O)NR—;
Ring A is phenylenyl or a 5 to 10-membered saturated or partially unsaturated monocyclic or bicyclic heterocyclylenyl or heteroarylenyl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
G is hydrogen, halogen, or

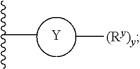

and
Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur.

In some embodiments, the present invention provides a compound of formula I-bb as a compound of any one of the following formulae:

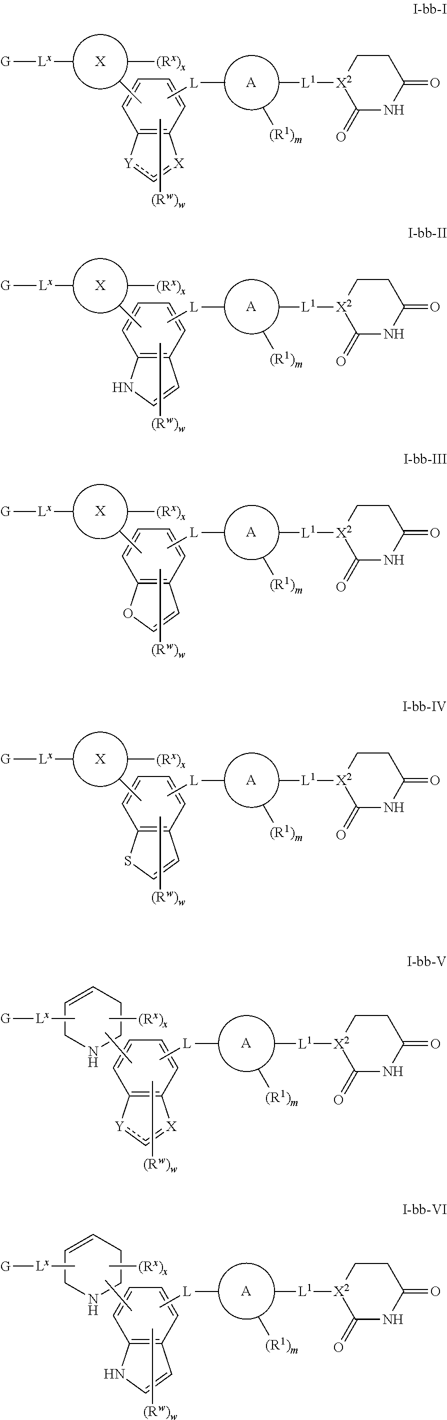

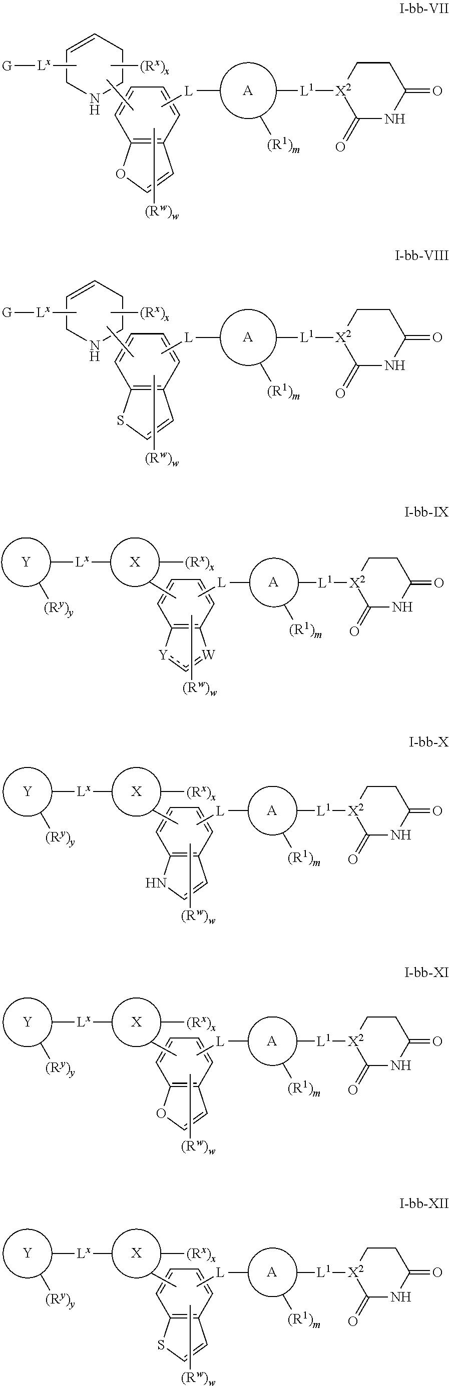

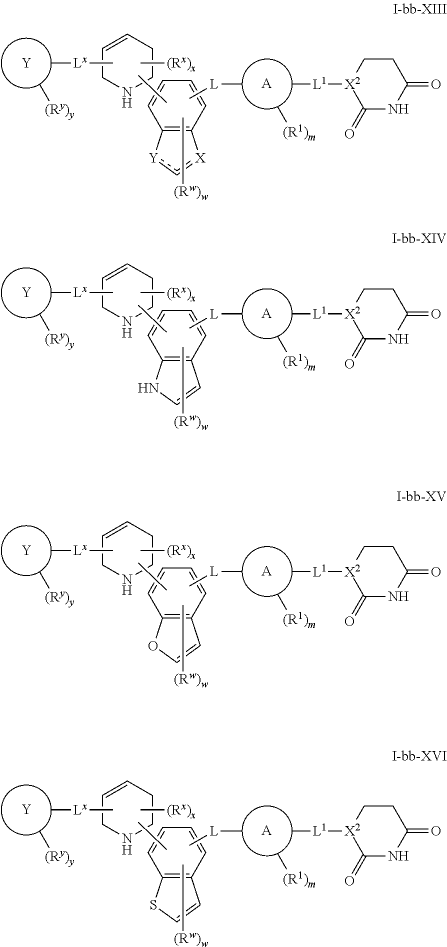

or a pharmaceutically acceptable salt thereof, wherein each variable is as defined above and described herein both individually and in combination.

In some embodiments, the present invention provides a compound of formula I-bb as a compound of any one of the following formulae:

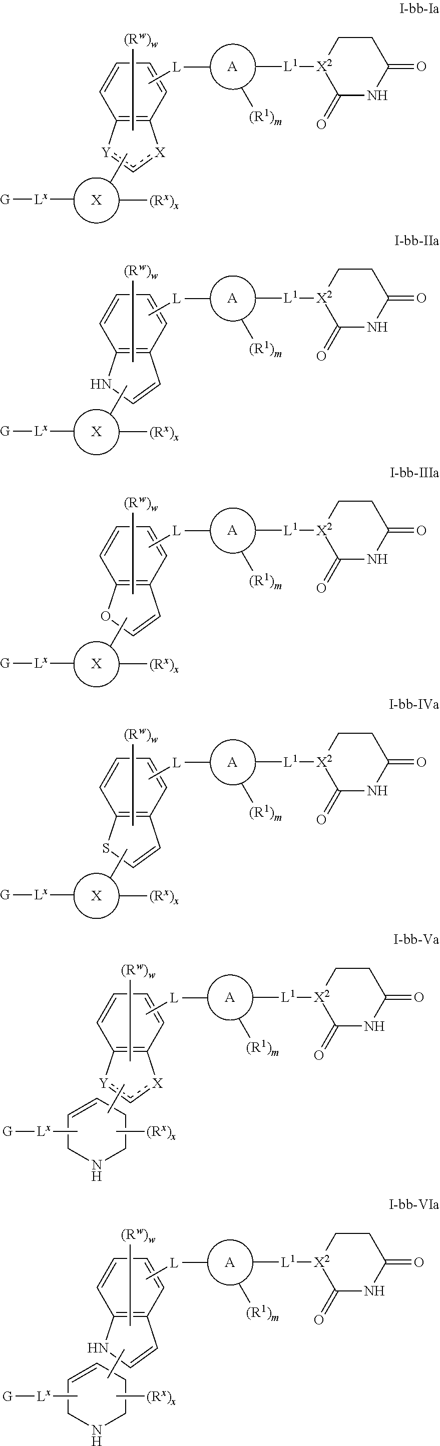

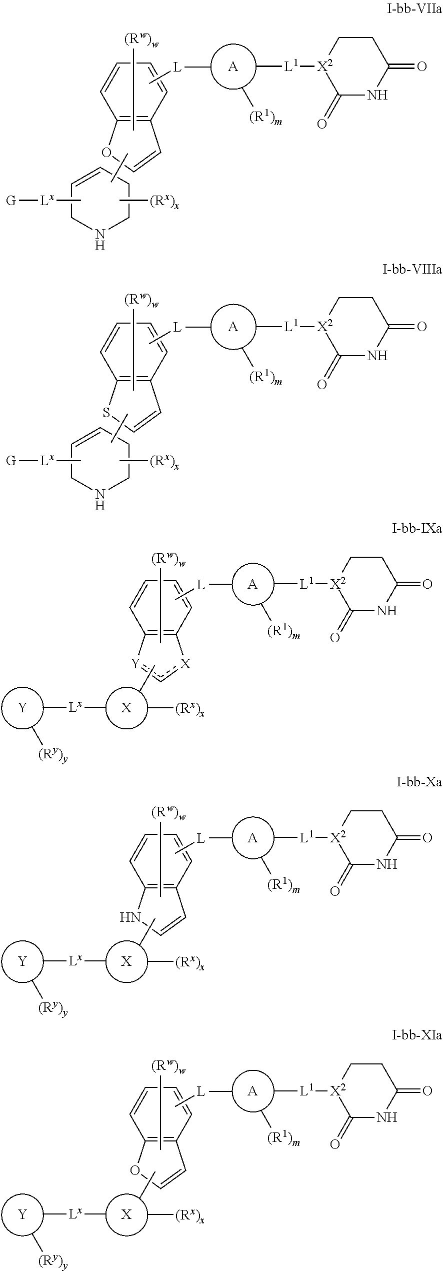

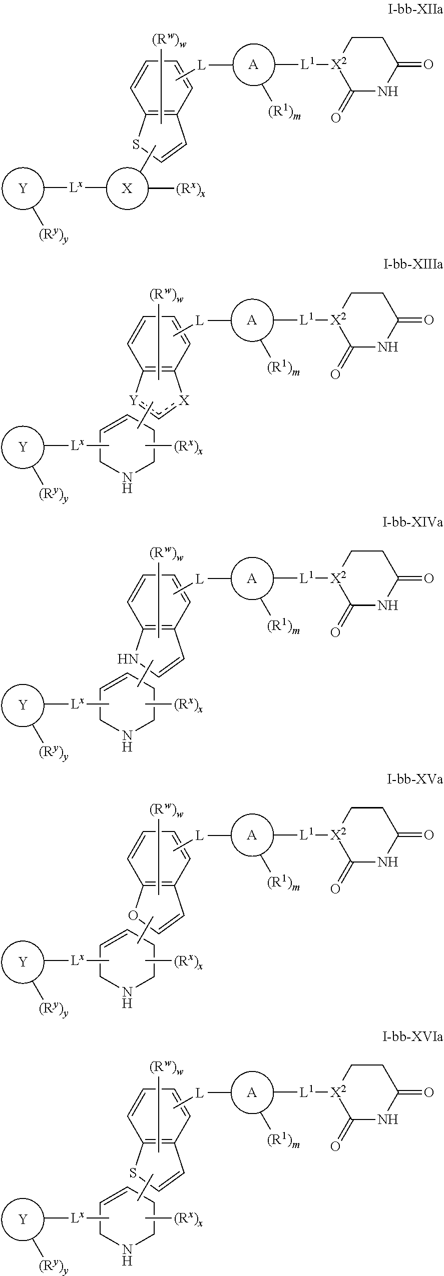

or a pharmaceutically acceptable salt thereof, wherein each variable is as defined above and described herein both individually and in combination.

In some embodiments, the present invention provides a compound of formula I-bb as a compound of any one of the following formulae:

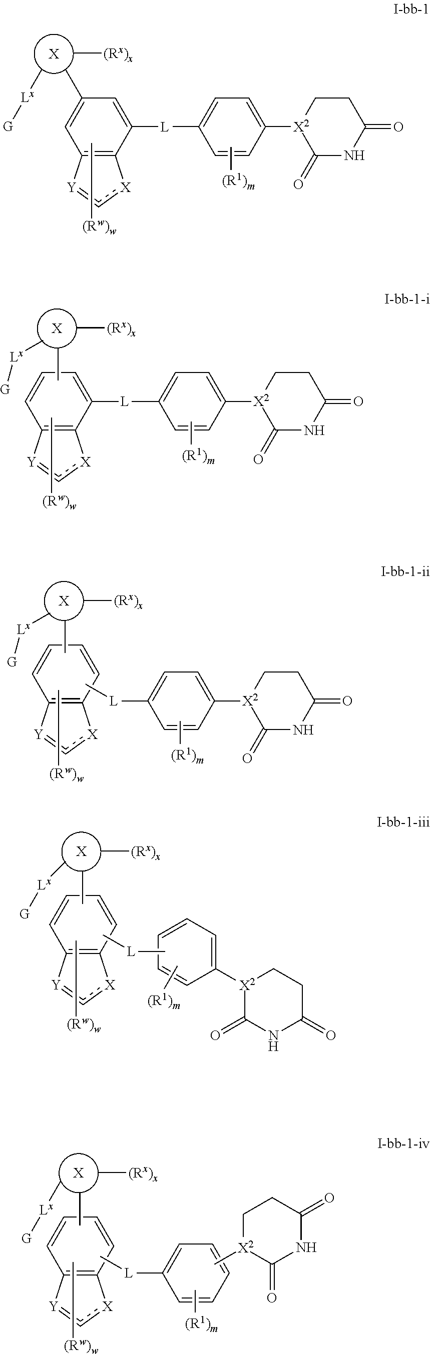

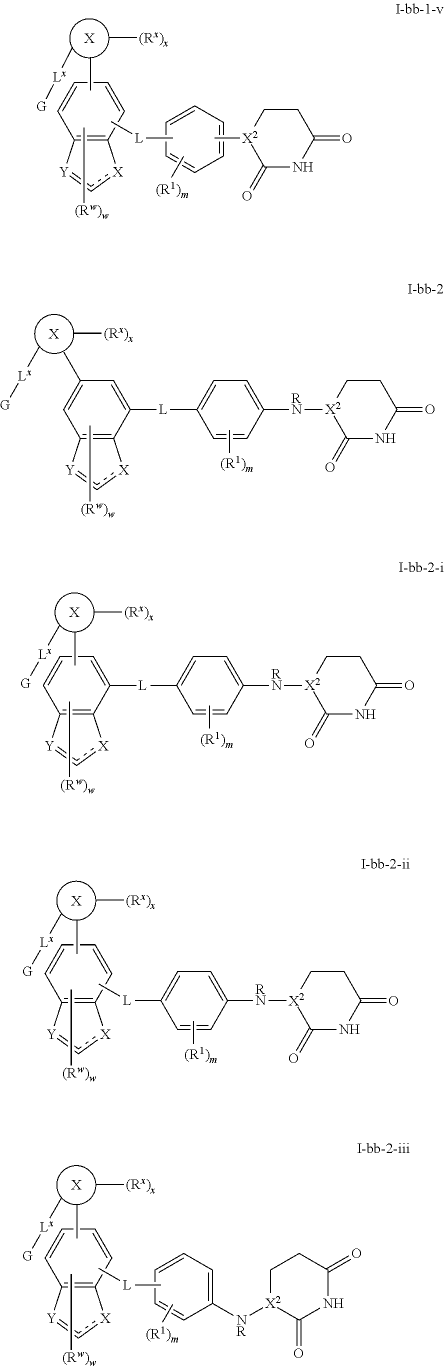

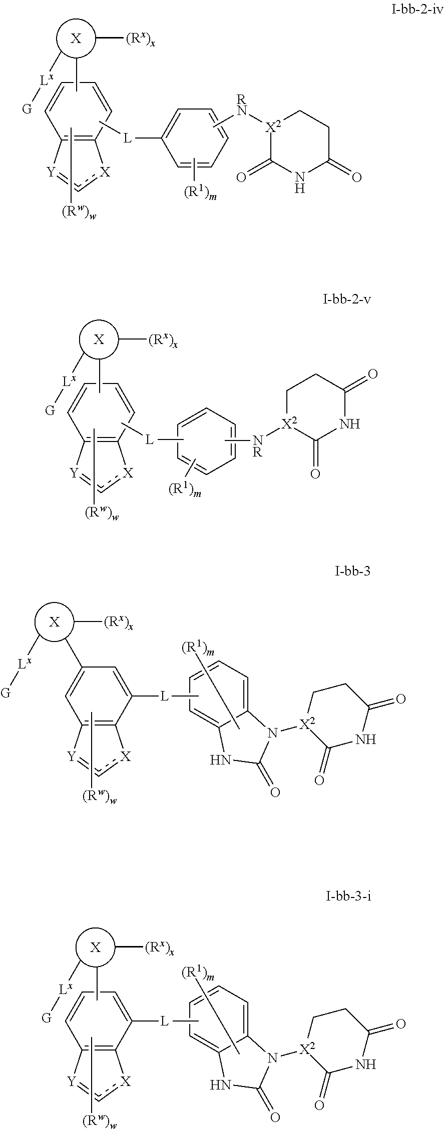

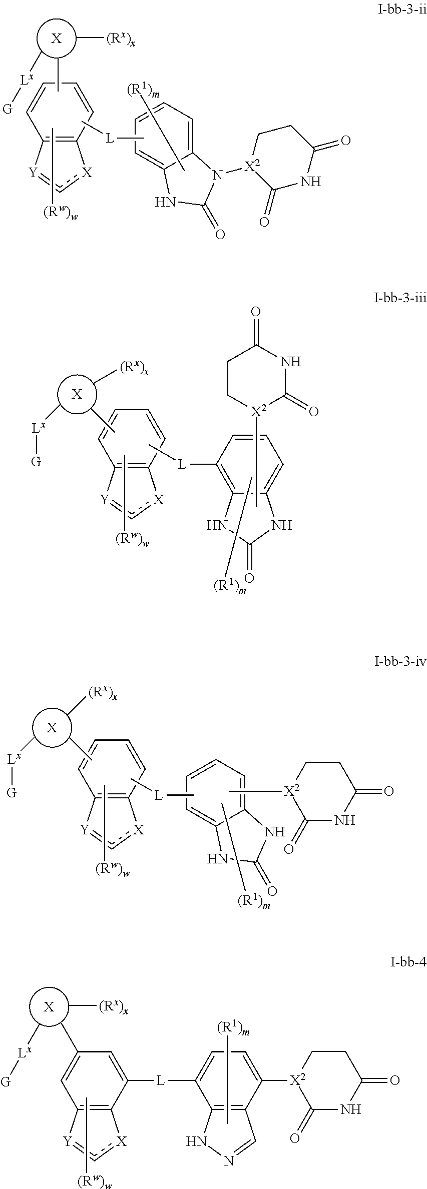

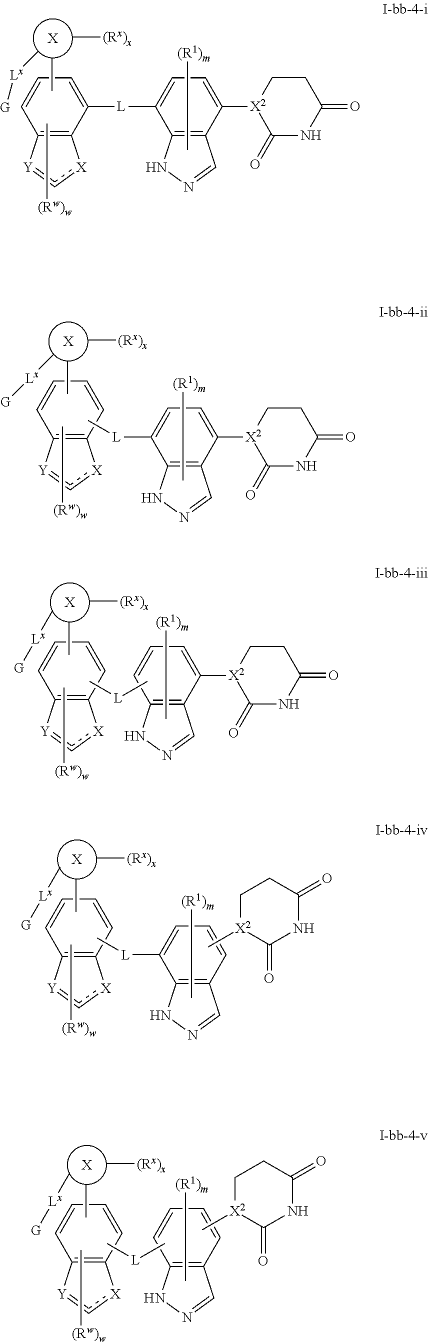

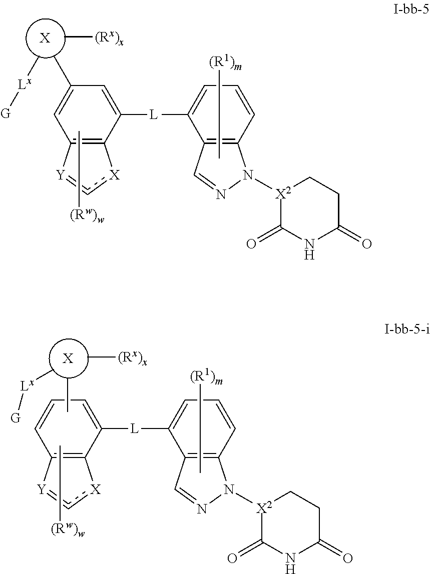

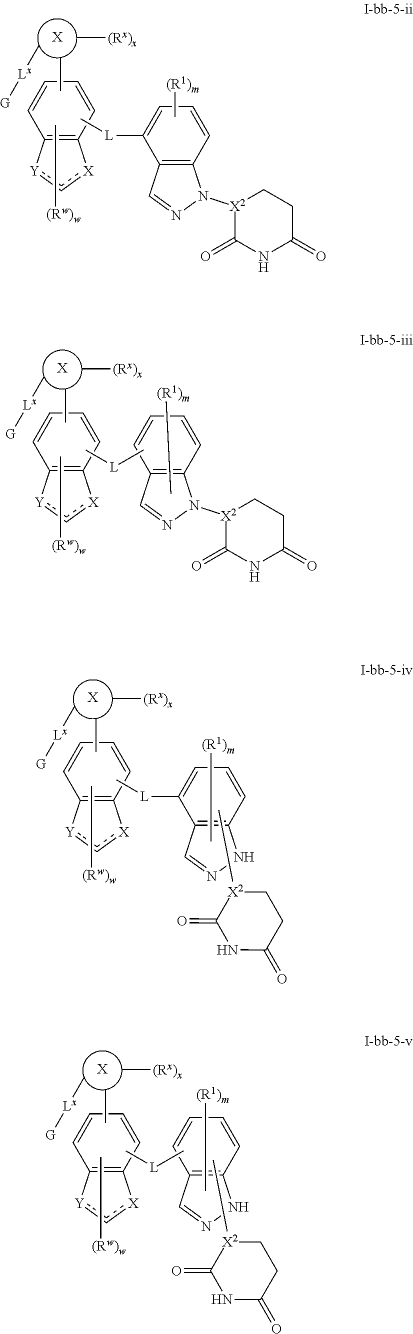

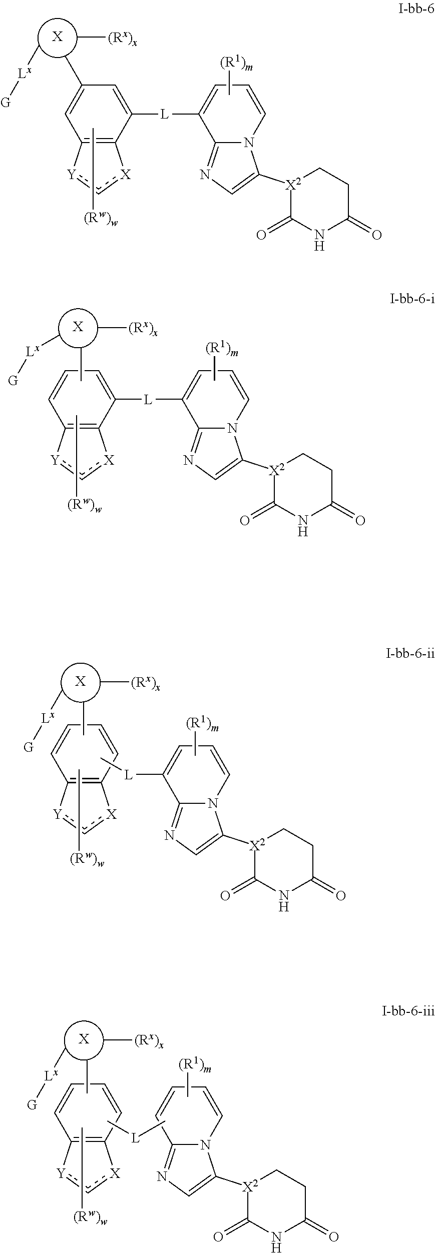

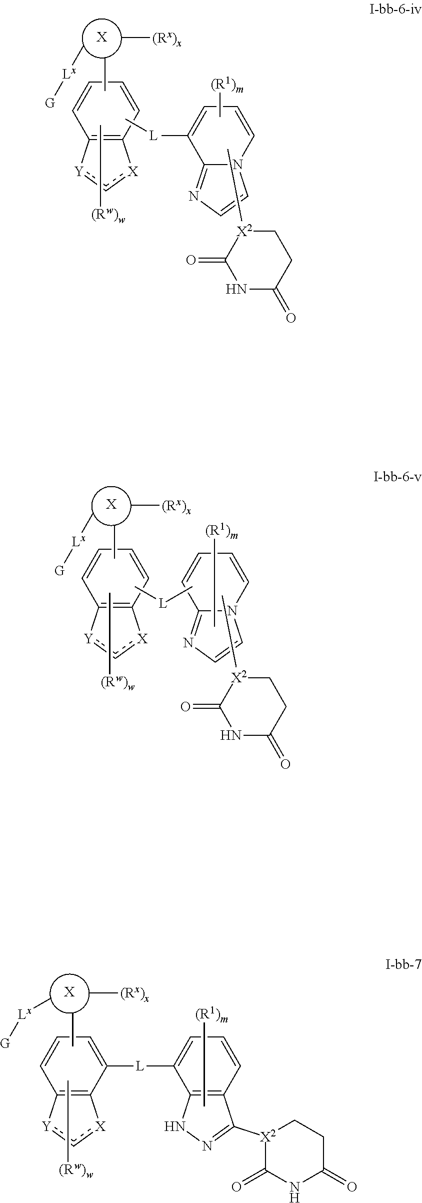

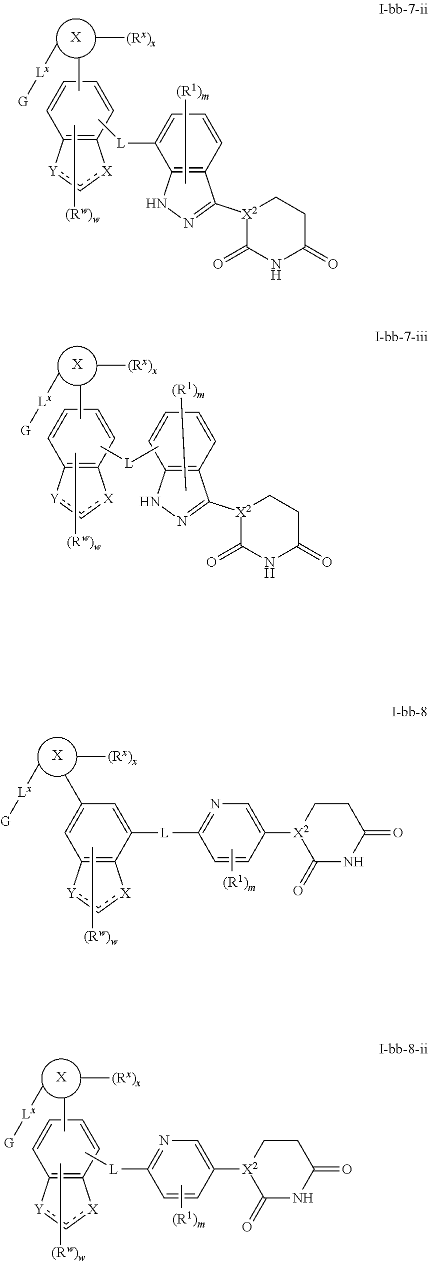

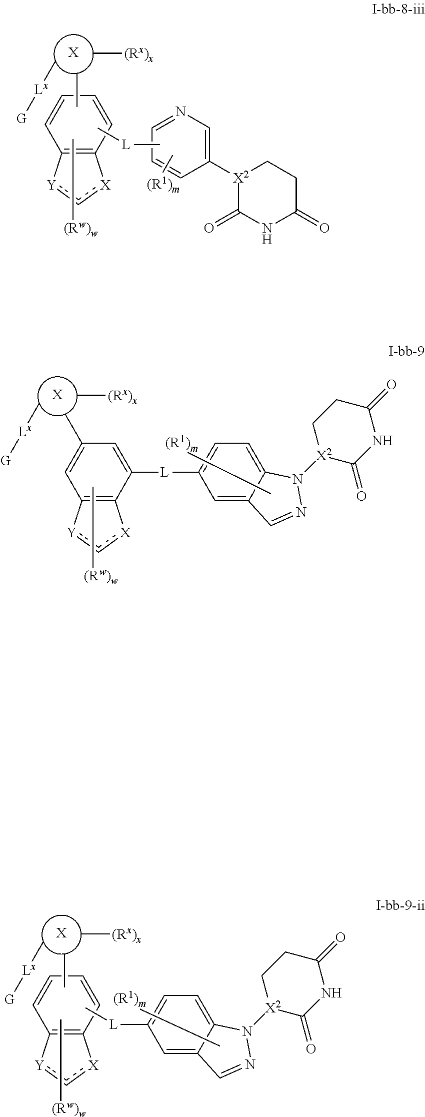

or a pharmaceutically acceptable salt thereof, wherein each variable is as defined above and described herein both individually and in combination.

In some embodiments, the present invention provides a compound of formula I-bb as a compound of any one of the following formulae:

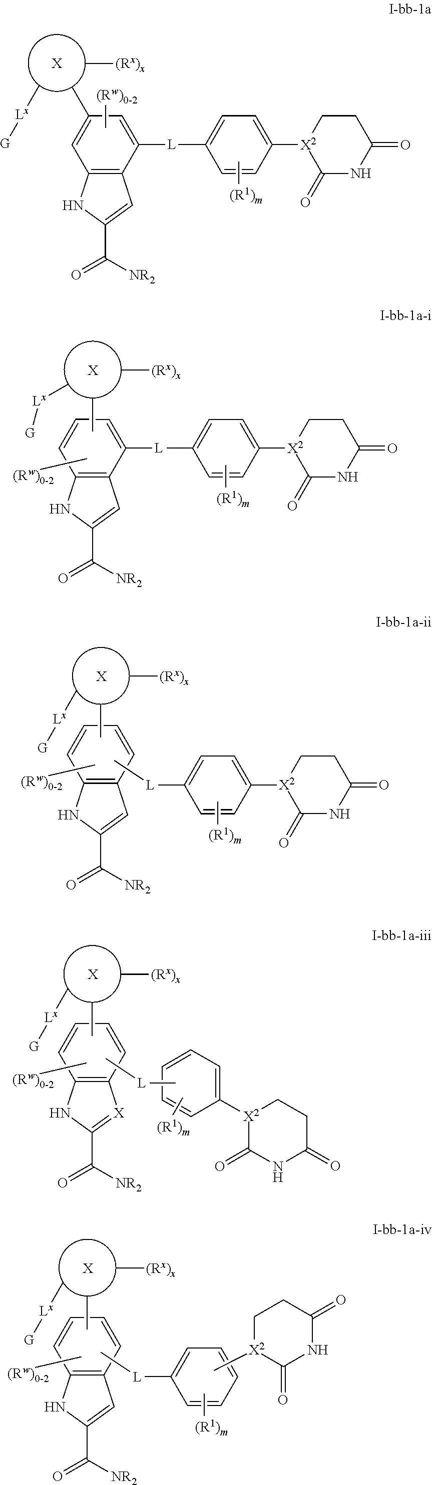

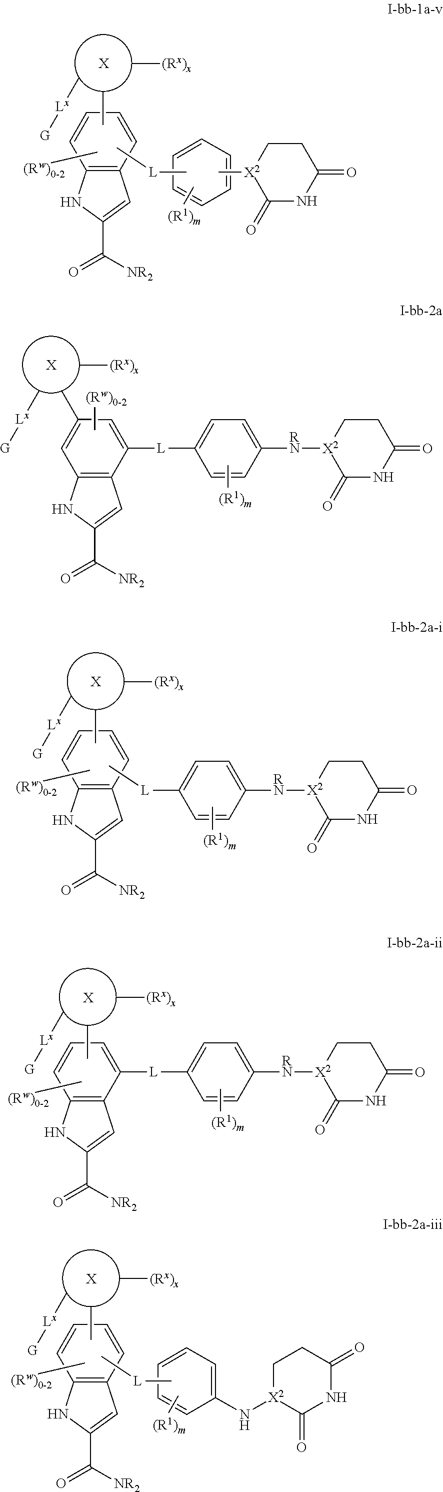

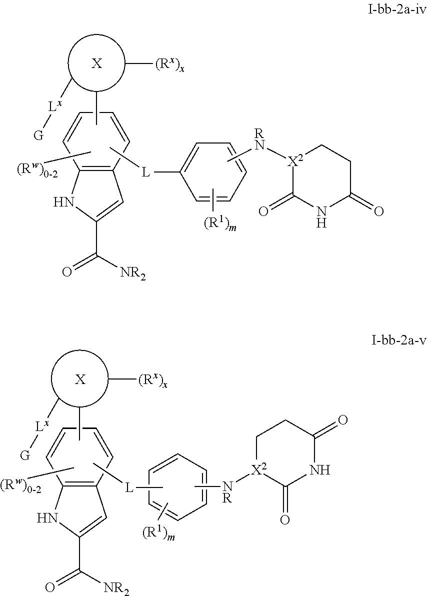

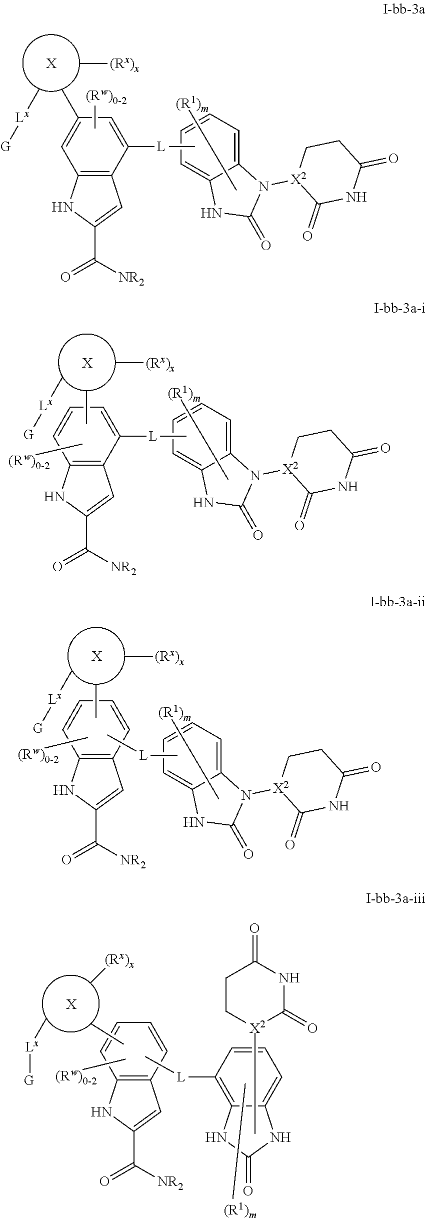

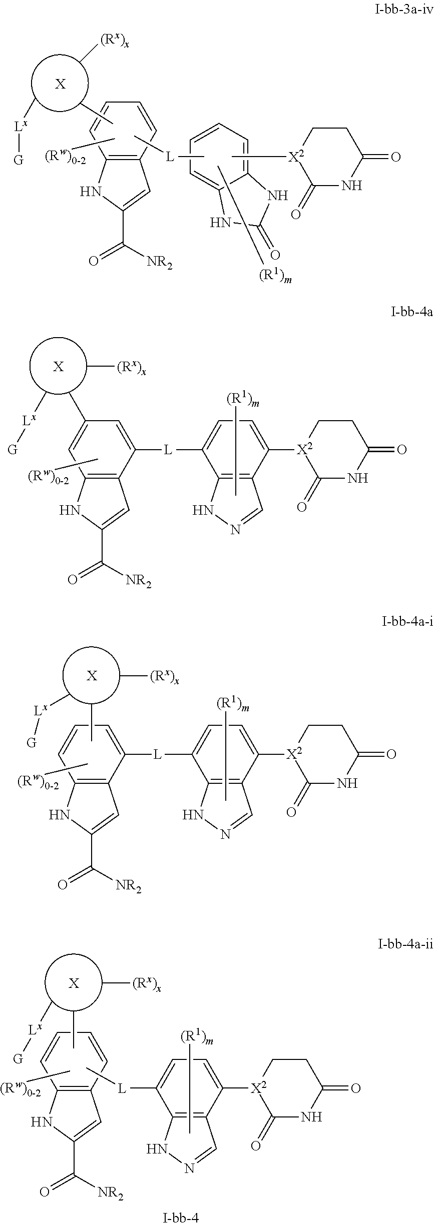

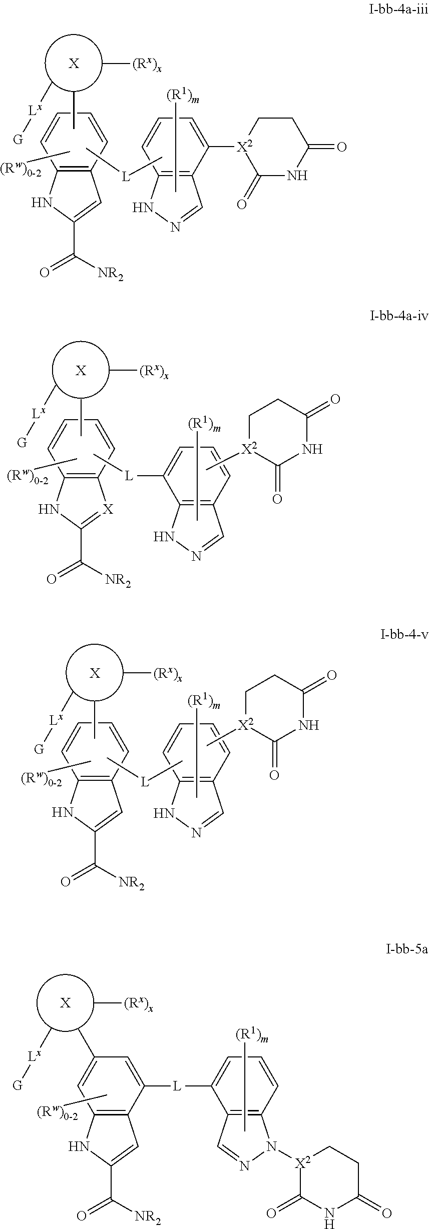

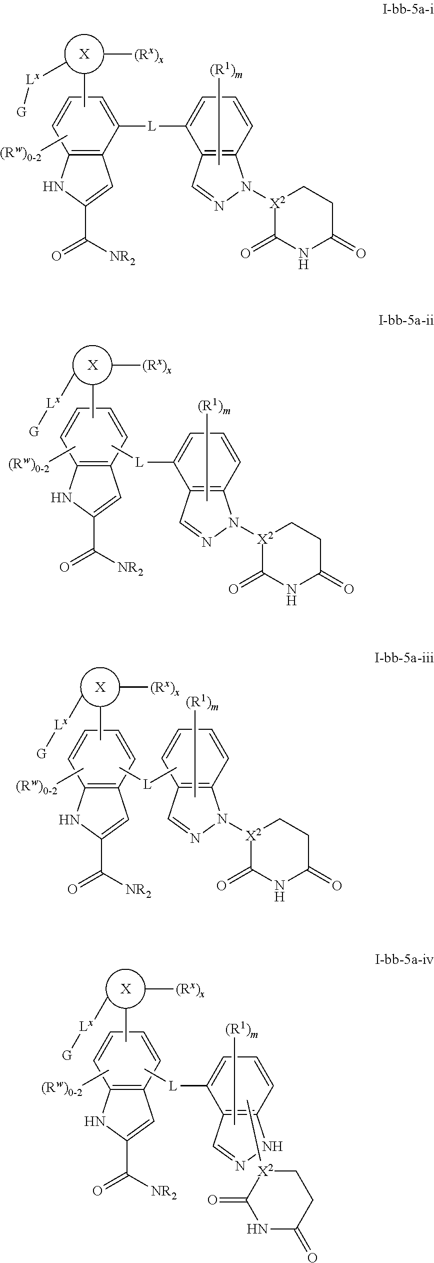

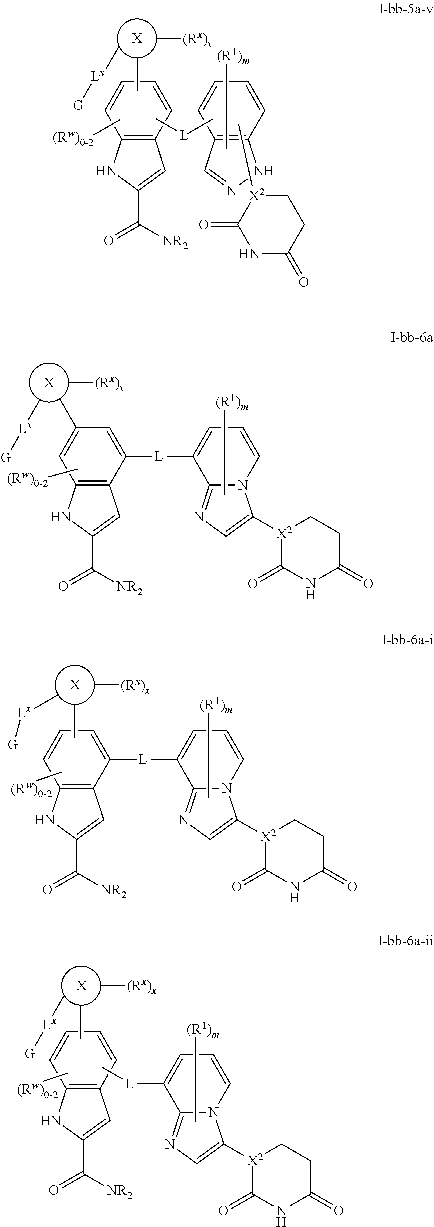

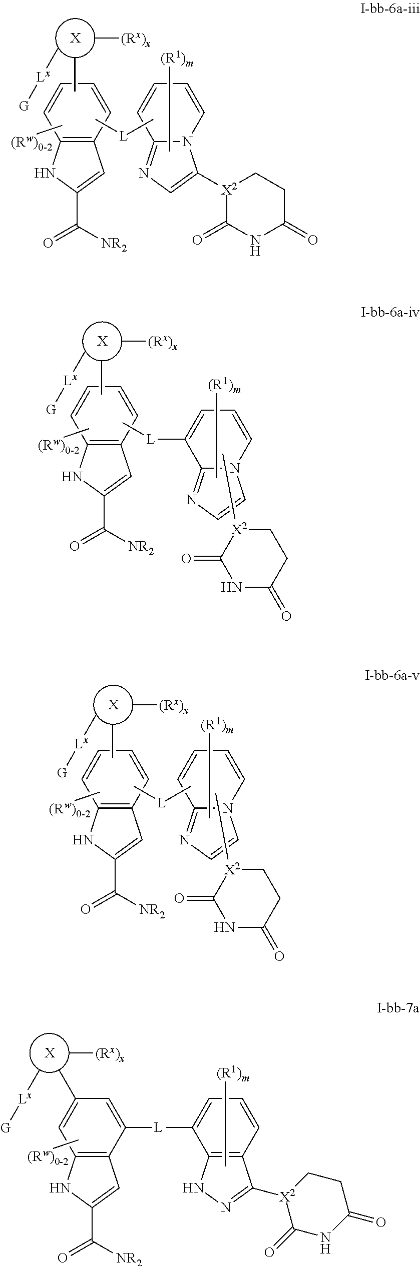

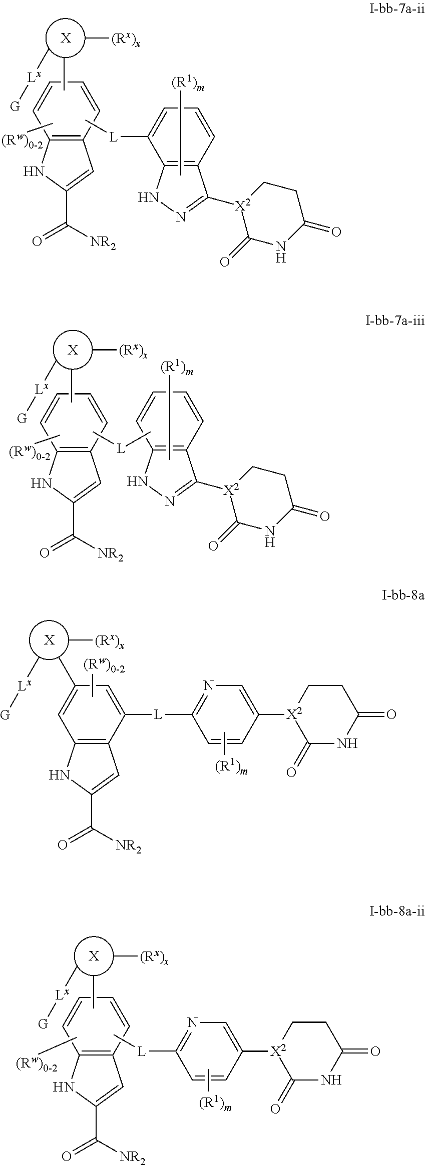

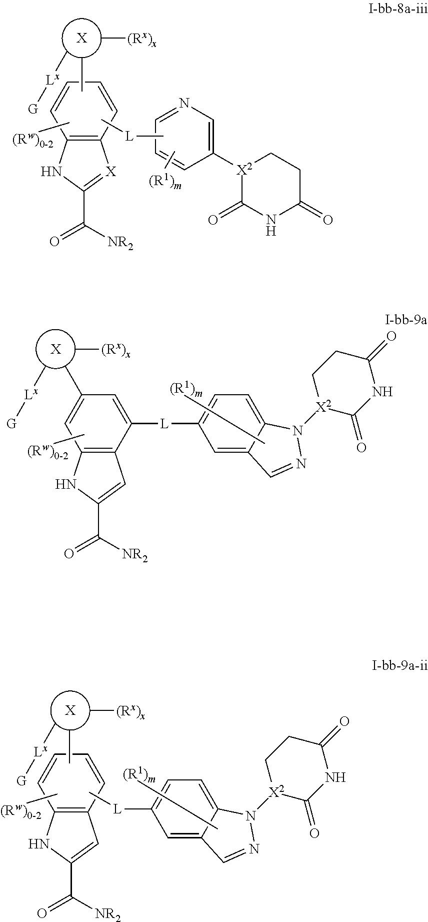

or a pharmaceutically acceptable salt thereof, wherein each variable is as defined above and described herein both individually and in combination.

In some embodiments, the present invention provides a compound of formula I-bb as a compound of any one of the following formulae:

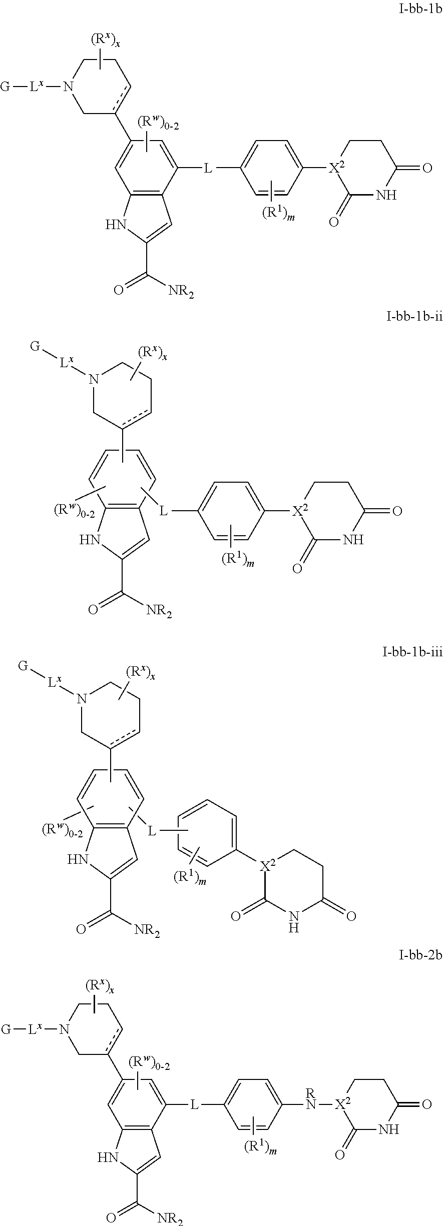

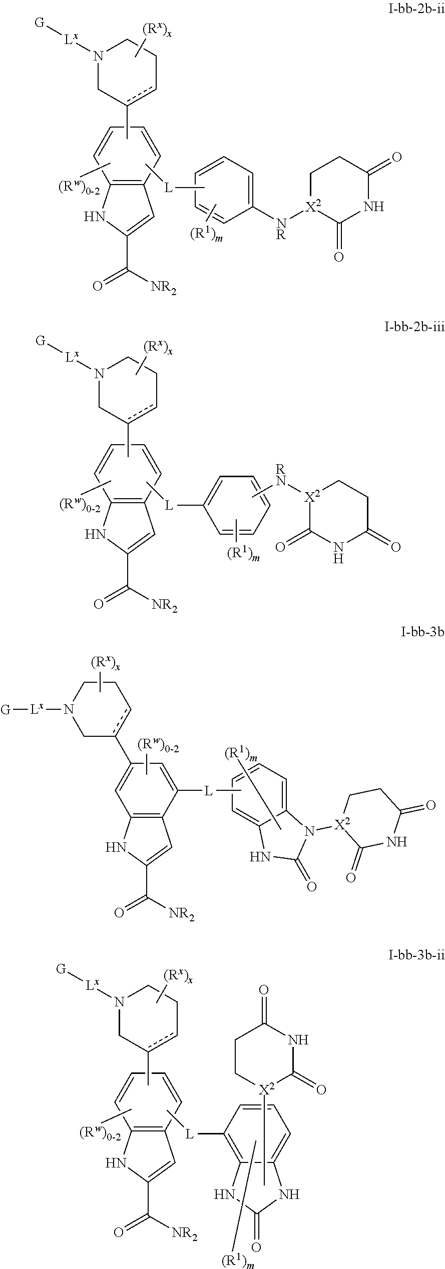

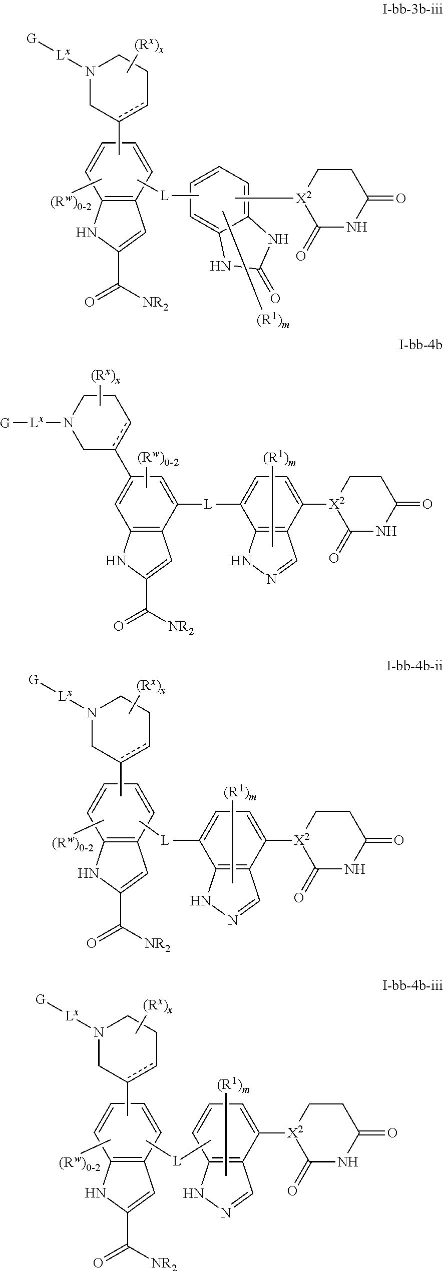

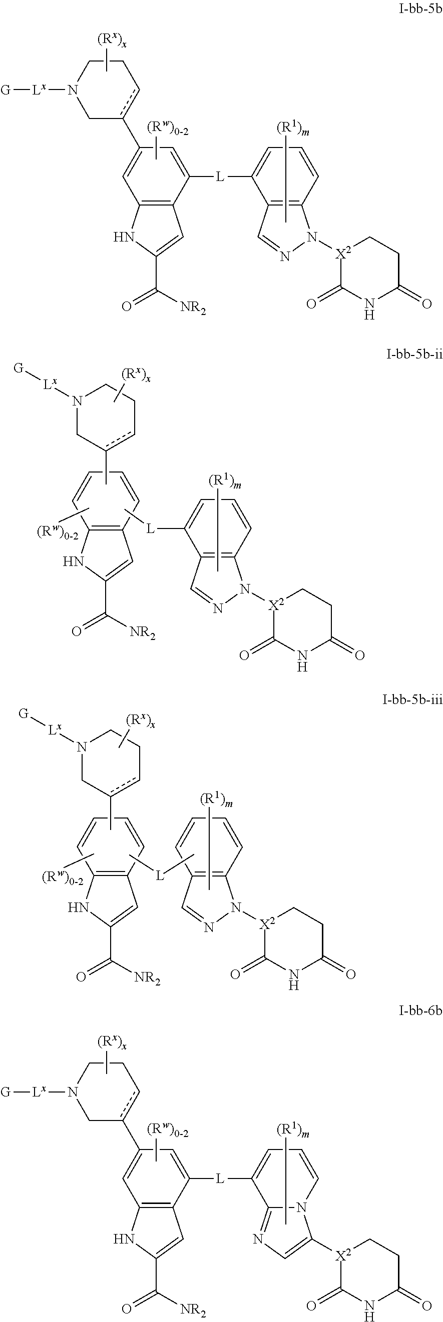

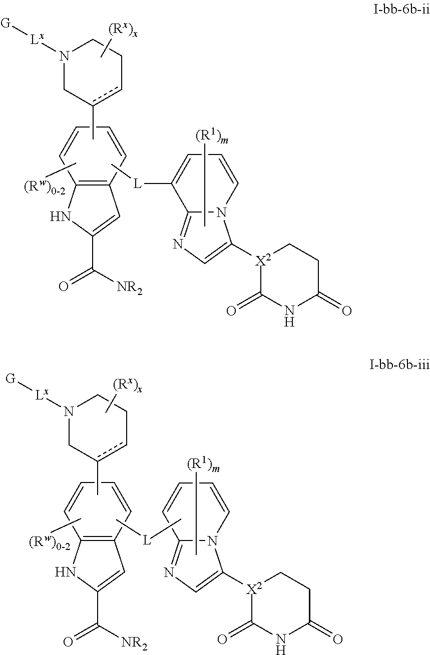

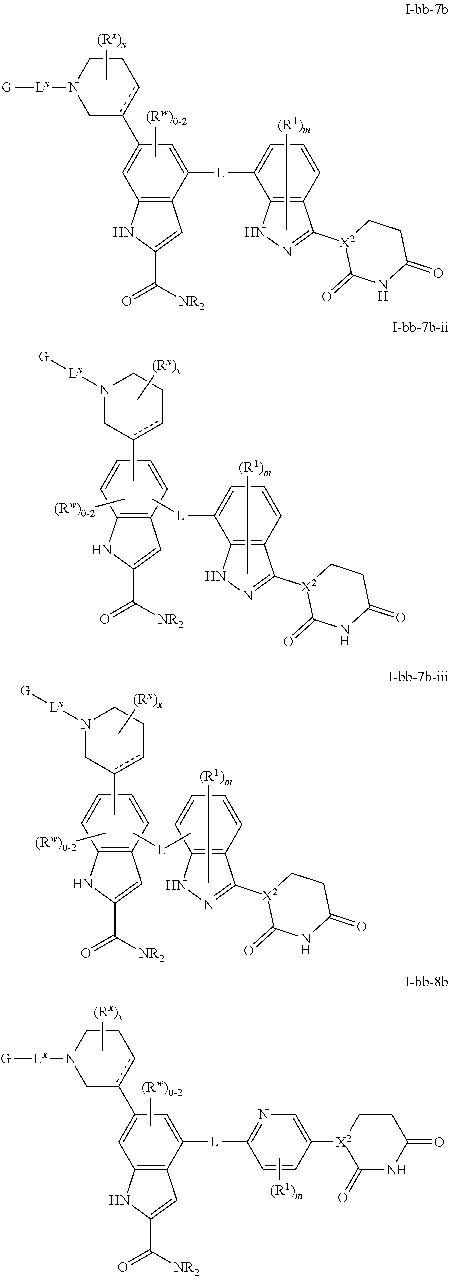

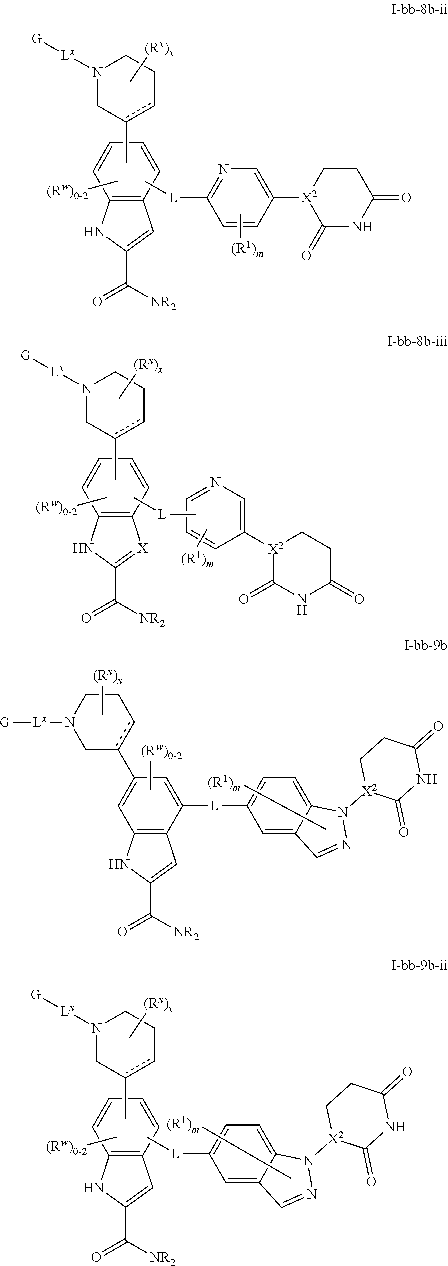

or a pharmaceutically acceptable salt thereof, wherein each variable is as defined above and described herein both individually and in combination, and wherein [symbol] represents a single or double bond.

In some embodiments, the present invention provides a compound of formula I-bb as a compound of any one of the following formulae:

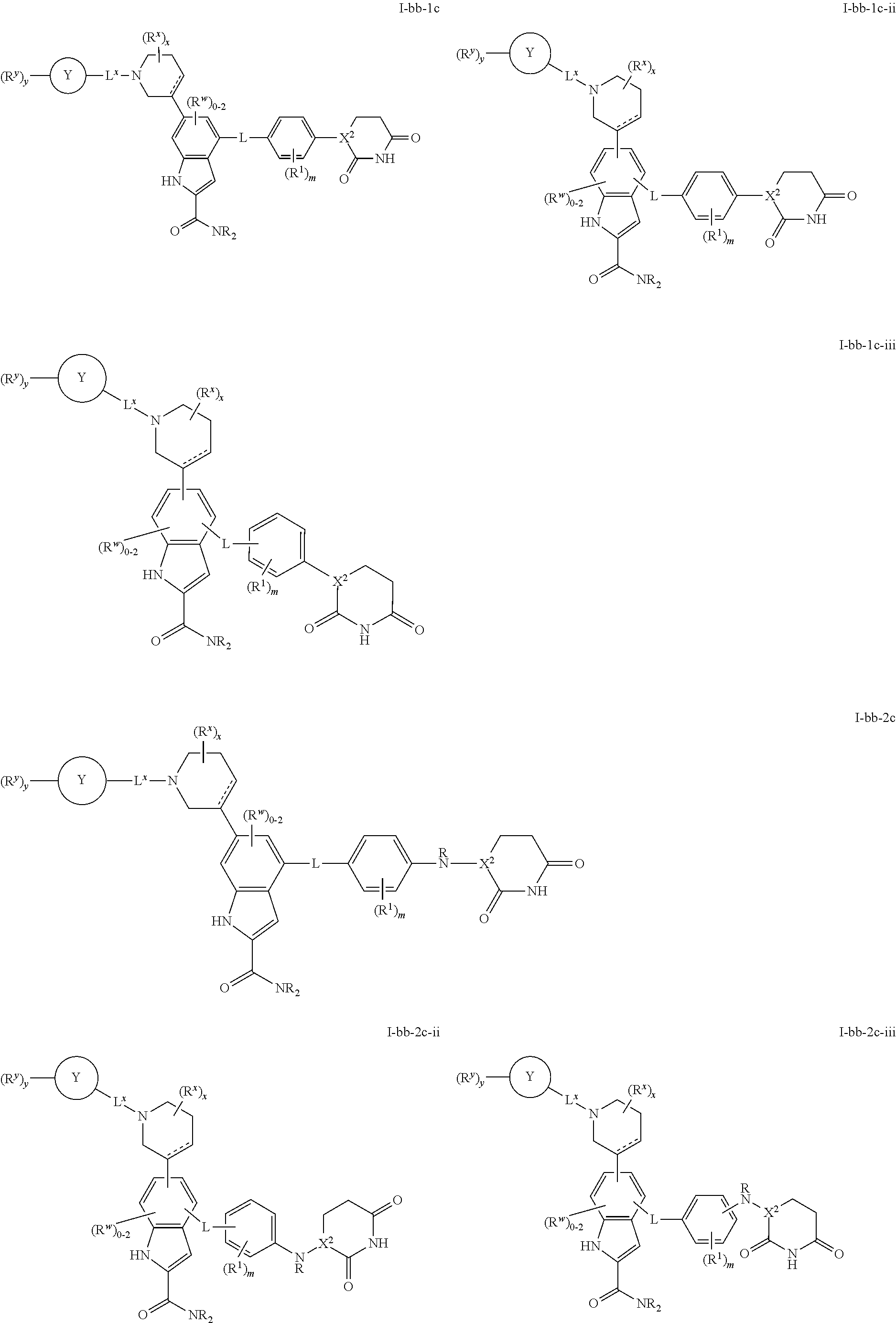

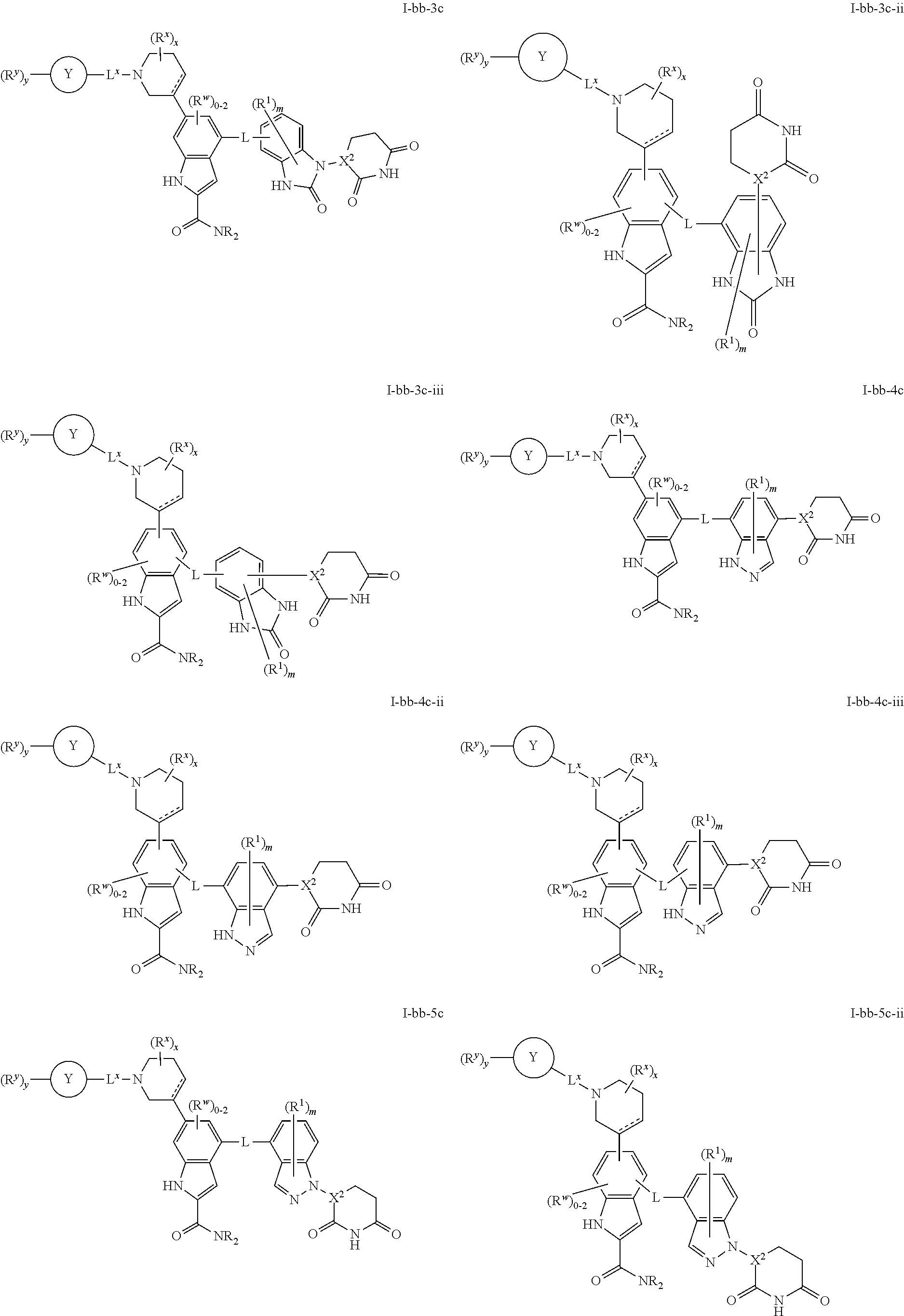

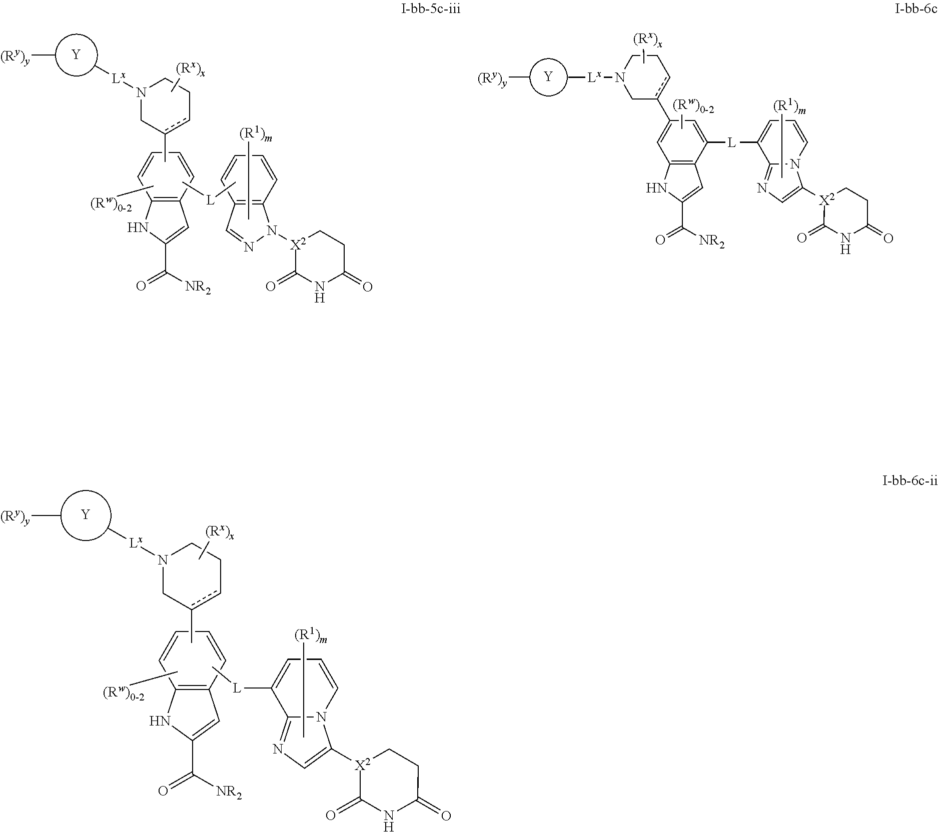

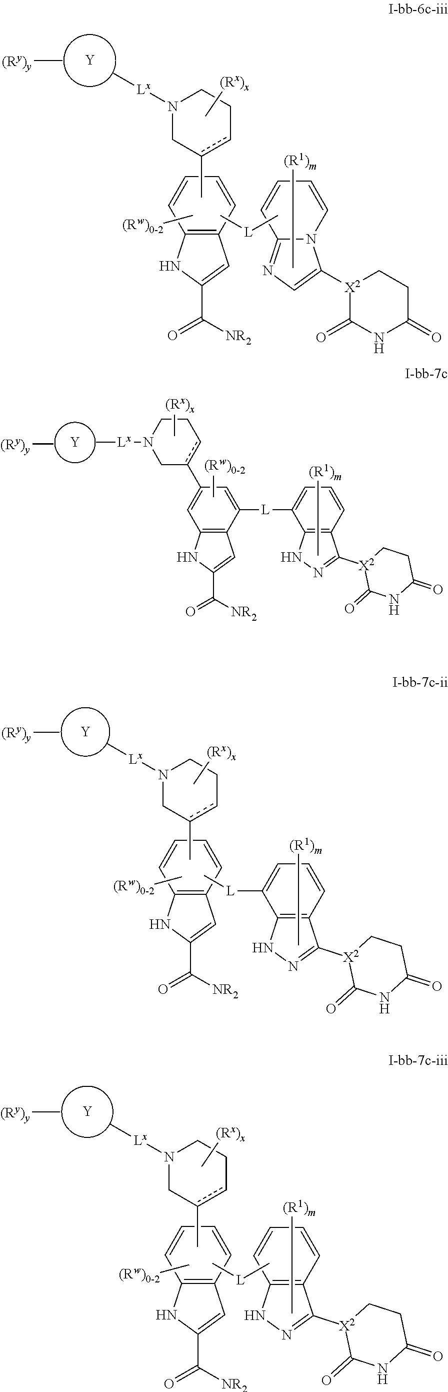

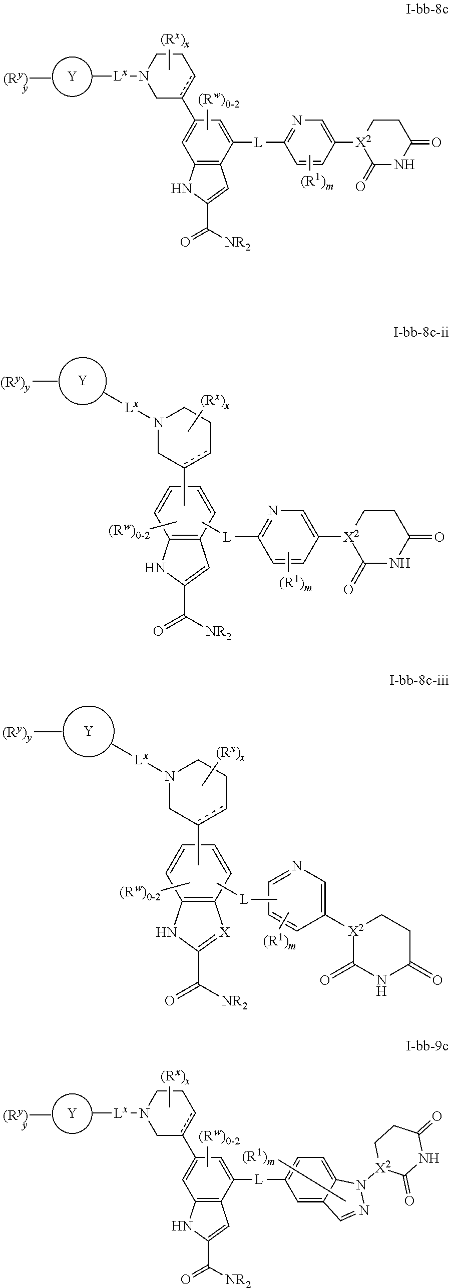

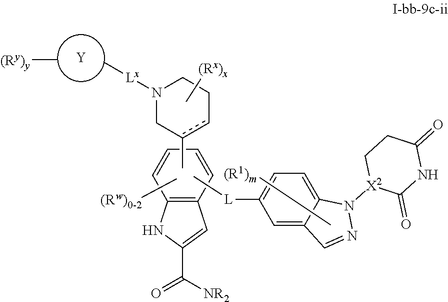

or a pharmaceutically acceptable salt thereof, wherein each variable is as defined above and described herein both individually and in combination.

In some embodiments, the present invention provides a compound of formula I-bb as a compound of any one of the following formulae:

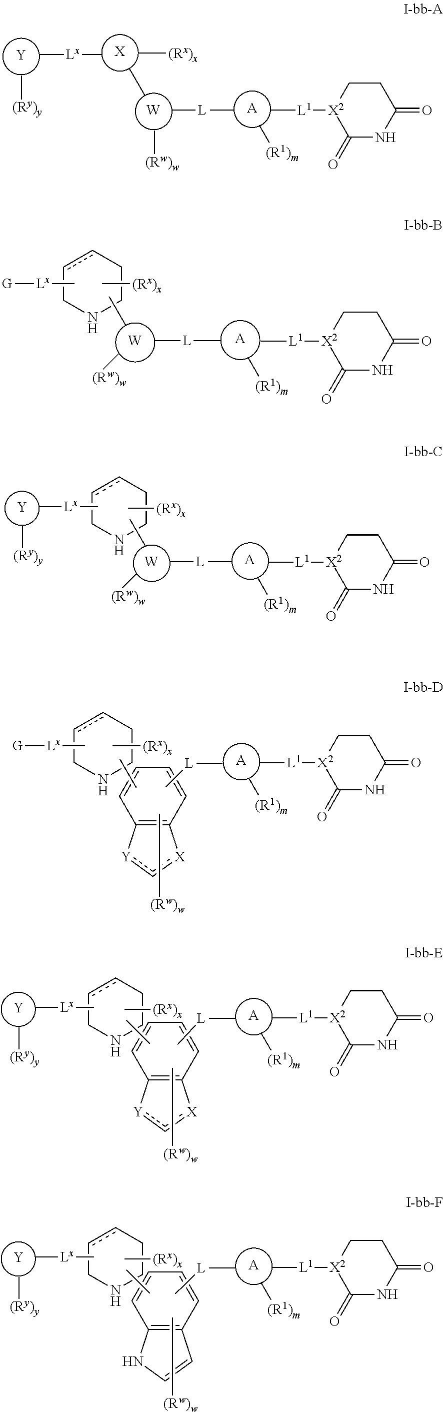

or a pharmaceutically acceptable salt thereof, wherein each variable is as defined above and described herein both individually and in combination:

In some embodiments, the present invention provides a compound of any one of the following formulae:

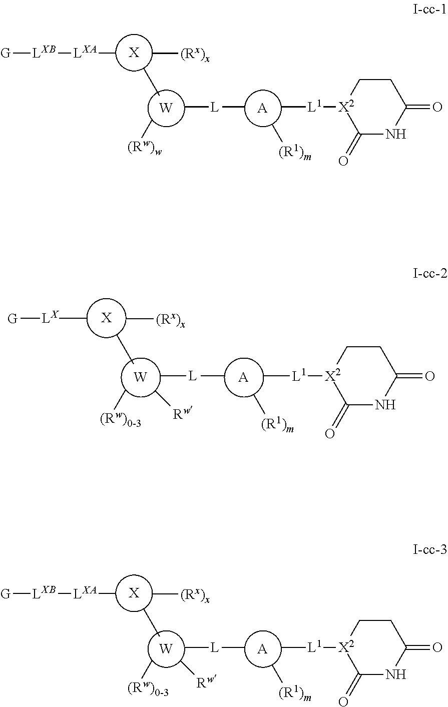

or a pharmaceutically acceptable salt thereof, wherein each Ring W, Ring X, R^{w}, R, w, x, G, L, L^{x}, Ring A. R¹, m, L¹, L^{XA}, L^{XB}, and X² is as defined above and described herein both individually and in combination, and wherein:
R^{w} is R^{w}, wherein R^{w} as defined above and described herein.

In some embodiments, R^{w} is —S(O)R, —S(O)₂R, —S(O)(NR)R—S(O)₂NR₂, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, —NRS(O)₂R, or an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur.

In some embodiments, R^{w} is —S(O)₂R. In some embodiments, R^{w} is —S(O)₂NR₂. In some embodiments, R^{w} is —S(O)(NR)R. In some embodiments, R^{w} is —S(O)R. In some embodiments, R^{w} is —C(O)R. In some embodiments, R^{w} is —C(O)OR. In some embodiments, R^{w} is —C(O)NR₂. In some embodiments, R^{w} is —C(O)NROR. In some embodiments, R^{w} is —OC(O)R. In some embodiments, R^{w} is —OC(O)NR₂. In some embodiments, R^{w} is —P(O)R₂. In some embodiments, R^{w} is —P(O)(OR)₂. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —NRC(O)OR. In some embodiments, R^{w} is —NRC(O)R. In some embodiments, R^{w} is —NRC(O)N(R)₂. In some embodiments, R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is —C(O)NHR. In some embodiments, R^{w} is —C(O)NHR, wherein R of R¹ is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{w} is optionally substituted C₁₋₆ aliphatic. In some embodiments, R^{w} is C₁₋₆ aliphatic, optionally substituted with —C(O)N(R^{o})₂. In some embodiments, R^{w} is C₁₋₆ aliphatic, optionally substituted with —NR^{o}(O)N(R^{o})₂. In some embodiments, R^{w} is

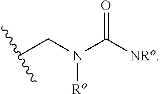

In some such embodiments, R^{o} is hydrogen or C₁₋₆ aliphatic. In some embodiments, R^{w} is C₁₋₆ aliphatic optionally substituted with —OR^{o}, wherein R^{o} is hydrogen or C₁₋₆ aliphatic. In some embodiments, R^{w} is C₁₋₆ aliphatic optionally substituted with halogen (e.g., fluoro). In some embodiments, R¹ is —CH₂F, —CHF₂, or —CF₃.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 5-6 membered saturated or partially unsaturated carbocyclic or heterocyclic ring having 1-2 heteroatoms independently selected from nitrogen, oxygen, and sulfur.

In some embodiments, R^{w} is an optionally substituted

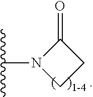

In some embodiments, R^{w} is an optionally substituted

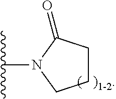

In some embodiments, R^{w} is an optionally substituted

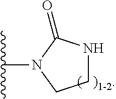

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{W} is an optionally substituted pyrazolyl, imidazolyl, triazolyl, or tetrazolyl. In some embodiments, R^{w} is an optionally substituted imidazolyl, optionally substituted with —C(O)N(R^{o})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{o})₂.

In some embodiments R^{W} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{W} is optionally substituted pyridinyl, pyrimidinyl, pyridazinyl, or triazinyl. In some embodiments, R^{w} is an optionally substituted pyridinonyl, pyrazinonyl, or pyrimidinonyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R¹ is —C(S)R, —C(NR)R, —S(O)R, —S(O)₂R, —S(O)(NR)R—S(O)₂NR. —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R—S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —S(O)R, or —NRS(O)₂R. In some embodiments, R^{w} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{W} is —S(O)R, —S(O)₂R, —S(O)(NR)R—S(O)₂NR₂, —S(O)R, —C(S)R, —C(NR)R, —C(O)R, —C(O)OR, —C(O)NR₂, or —C(O)NROR. In some embodiments, R^{w} is —C(S)R, —C(NR)R, —C(O)R, —C(O)OR, or —C(O)NR₂.

In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w} is —C(O)R, wherein R is of R^{w} is optionally substituted C₁₋₆ aliphatic. In some embodiments, R^{w} is —C(O)R, wherein R is of R¹ is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OR, wherein R is of R^{w} is optionally substituted C₁₋₆ aliphatic. In some embodiments, R^{w} is —C(O)OR, wherein R is of R^{w} is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{W} is an optionally substituted 5-6 membered heteroaryl ring having 1-4 heteroatoms independently selected from nitrogen, oxygen, and sulfur.

In some embodiments, R^{w} is —C(O)NR₂, wherein each R is of R^{w} is independently hydrogen or an optionally substituted C₁₋₆ aliphatic. In some embodiments, R¹ is —C(O)NR₂, wherein each R is of R^{W} is independently hydrogen or an optionally substituted phenyl. In some embodiments, R^{W} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(NR)R. In some embodiments, R^{w} is C(NR)R, wherein each R is of R^{w} is independently hydrogen or an optionally substituted C₁₋₆ aliphatic. In some embodiments, R^{w} is C(NR)R, wherein each R is of R¹ is independently hydrogen or an optionally substituted phenyl. In some embodiments, R^{w} is C(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is C(NR)R, wherein each R is of R¹ is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments, R^{w} is —C(O)NHR, wherein R is methyl, ethyl, or cyclopropyl. In some embodiments, R^{w} is —C(O)NR₂, wherein the two R groups of R^{w} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)R, wherein R is of R¹ is optionally substituted C₁₋₆ aliphatic. In some embodiments, R^{w} is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R¹ is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{W} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{w} is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R is —NRC(O)OR, wherein each R is of R is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)z, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R¹ is independently hydrogen or an optionally substituted 5-6 membered heteroaryl ring having 1-4 heteroatoms independently selected from nitrogen, oxygen, and sulfur.

In some embodiments, R^{w} is —S(O)R, wherein R is of R¹ is optionally substituted C₁₋₆ aliphatic. In some embodiments, R^{w} is —S(O)R, wherein R is of R^{w} is optionally substituted phenyl. In some embodiments, R^{w} is —S(O)R, wherein R is of R^{W} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R¹ is —S(O)(NR)R, wherein each R is of R^{w} is independently hydrogen or optionally substituted phenyl. In some embodiments, R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R^{w} is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments, R^{w} is an optionally substituted:

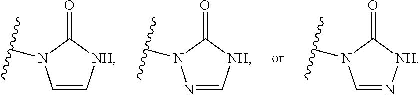 [SMILES: *n1cc[nH]c1=O ; *n1nc[nH]c1=O ; "or" ; *n1cn[nH]c1=O]

In some embodiments, R^{w} is:

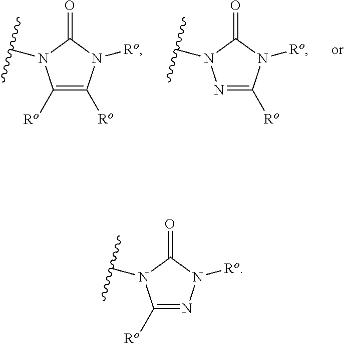

In some such embodiments, R^{o} is hydrogen or C₁₋₆ aliphatic. In some embodiments, R^{w} is:

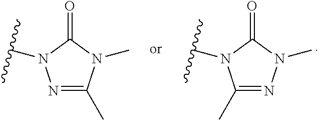

In some embodiments, R^{w} is an optionally substituted:

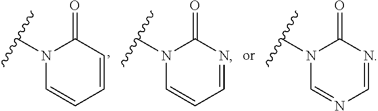

In some embodiments, R^{w} is:

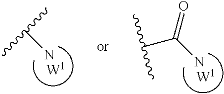

wherein Ring W¹ is as defined above and described herein.

In some embodiments, R^{w} is:

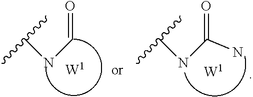

In some embodiments, R^{w} is:

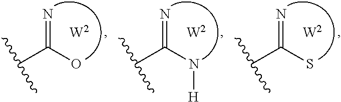

wherein Ring W² is as defined above and described herein.

In some embodiments, R^{w} is —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr,

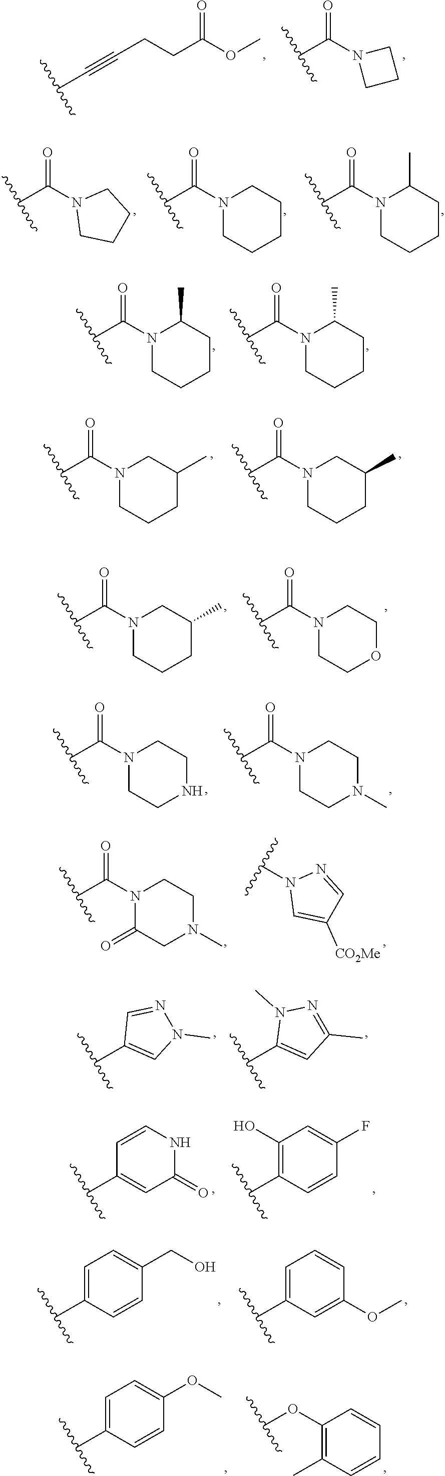

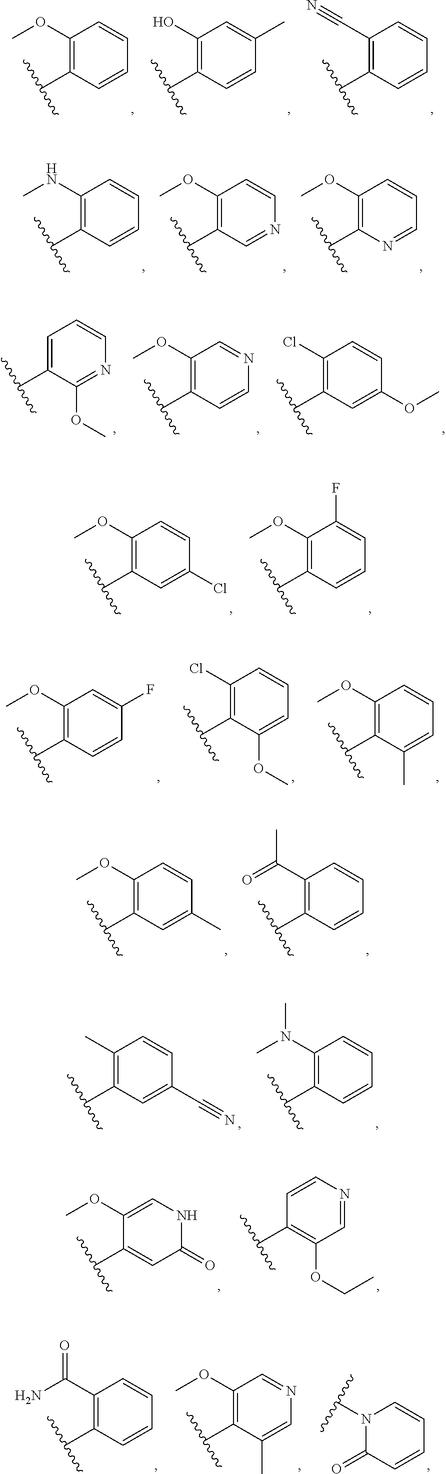

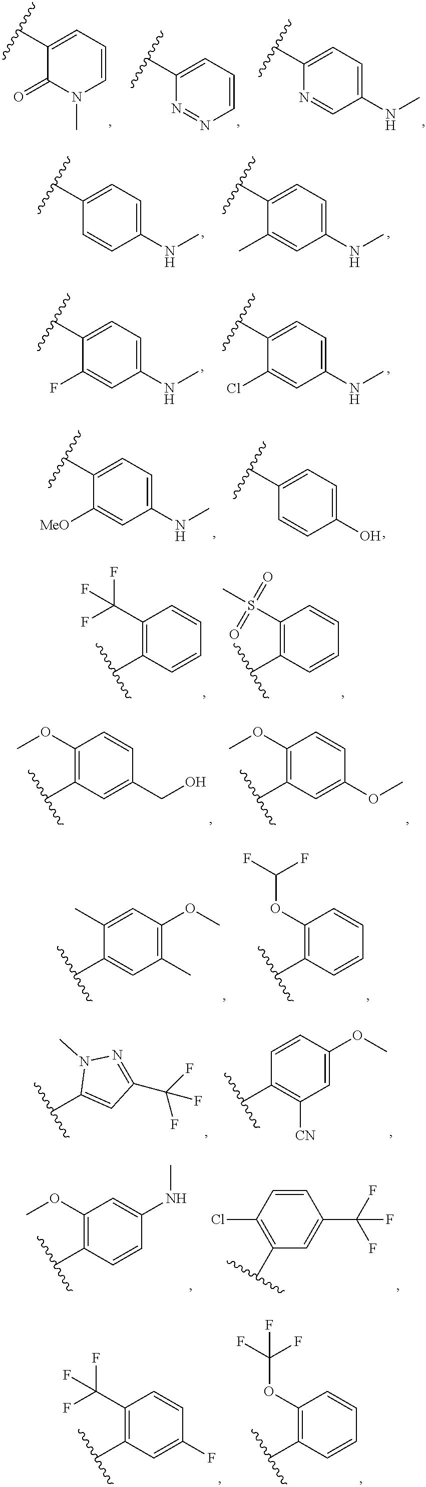

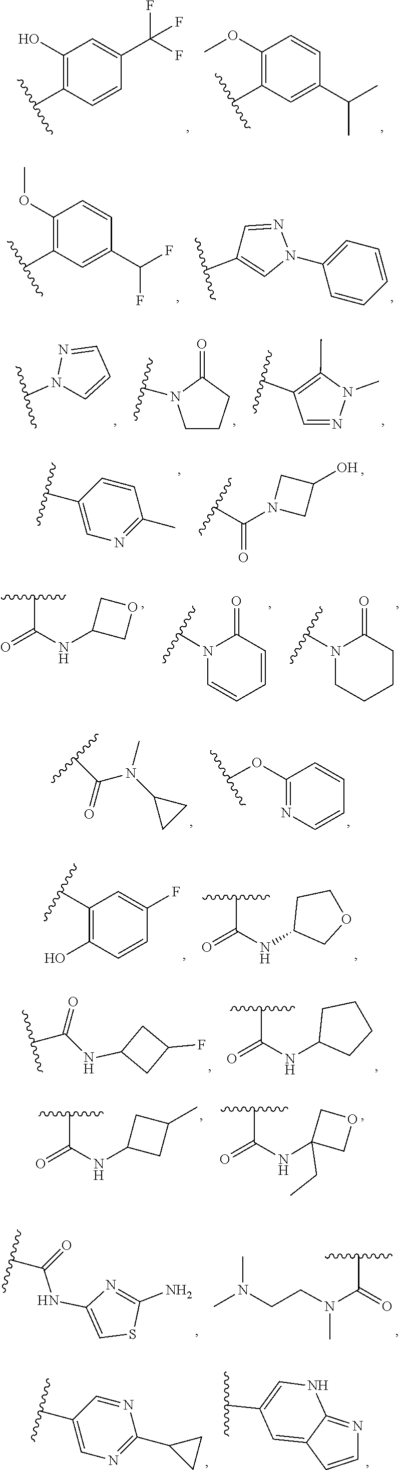

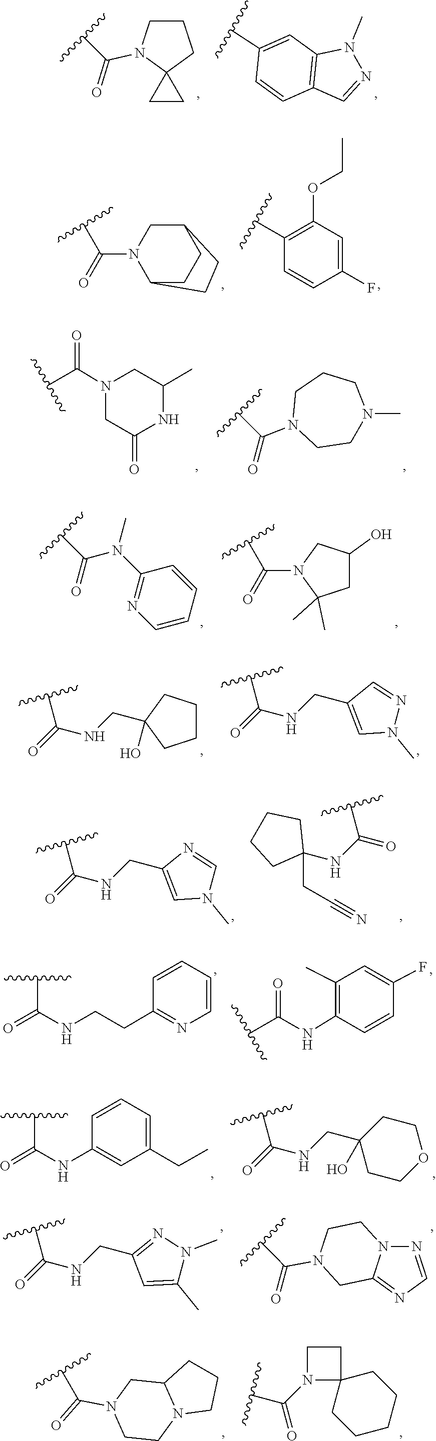

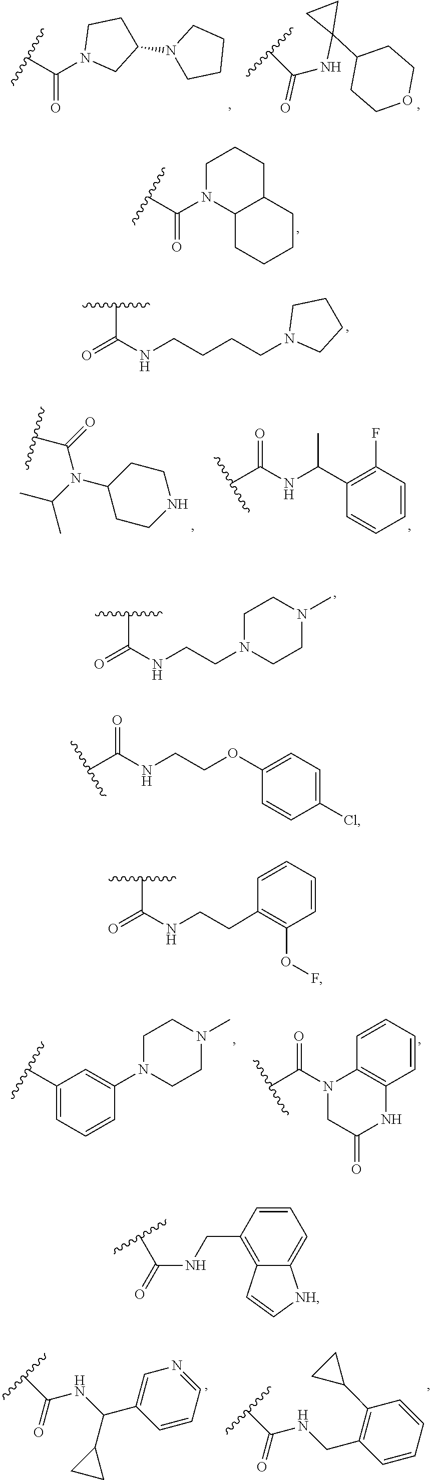

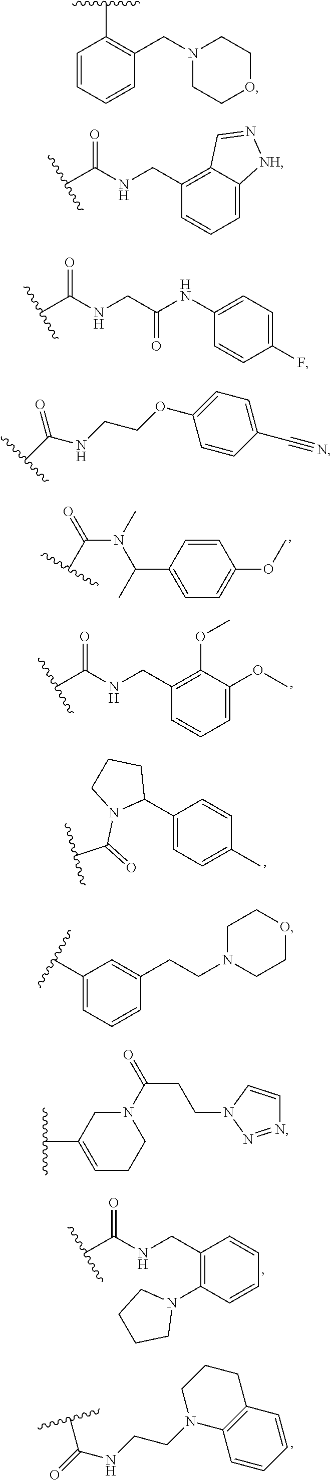

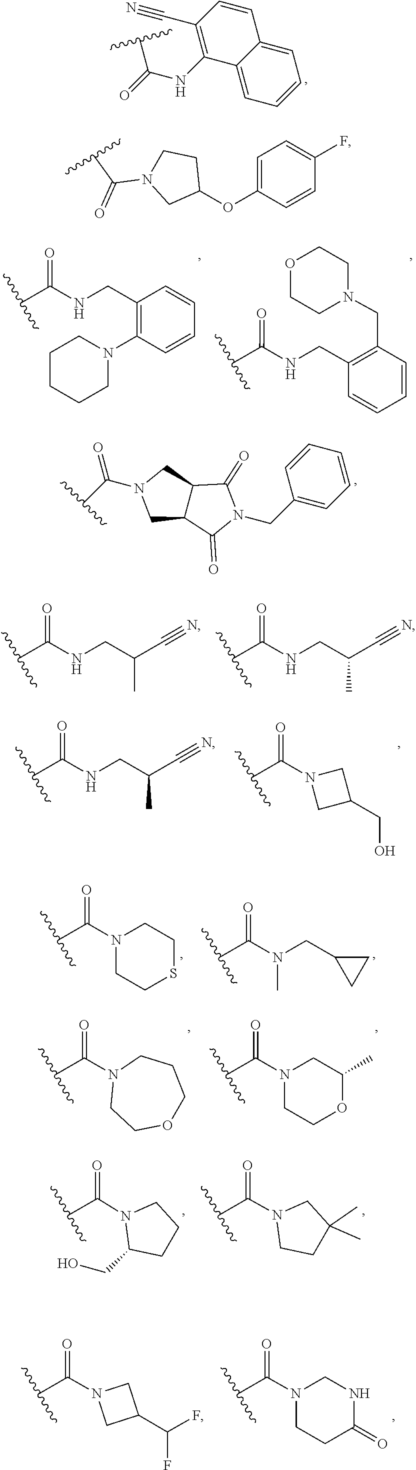

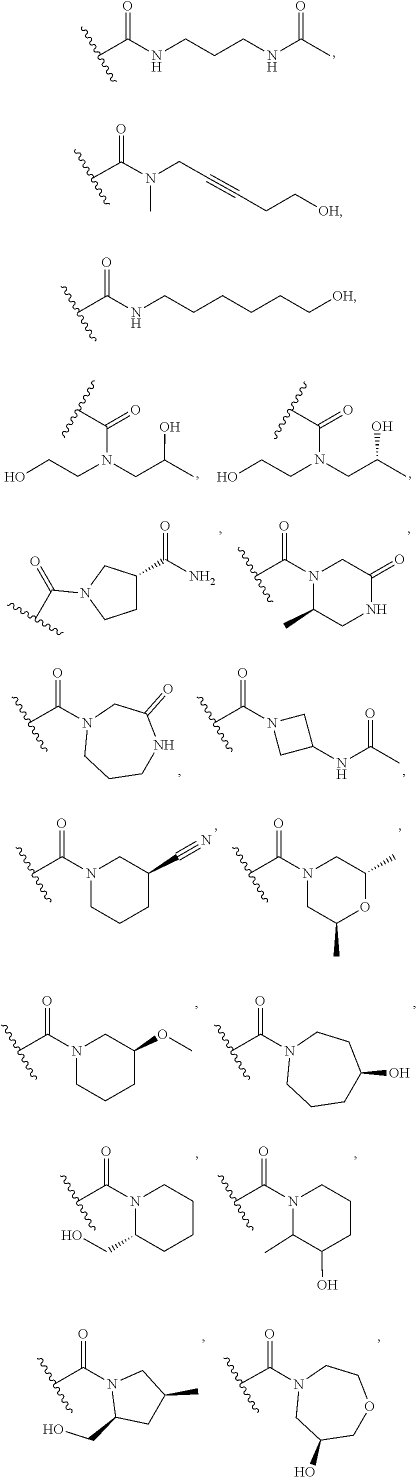

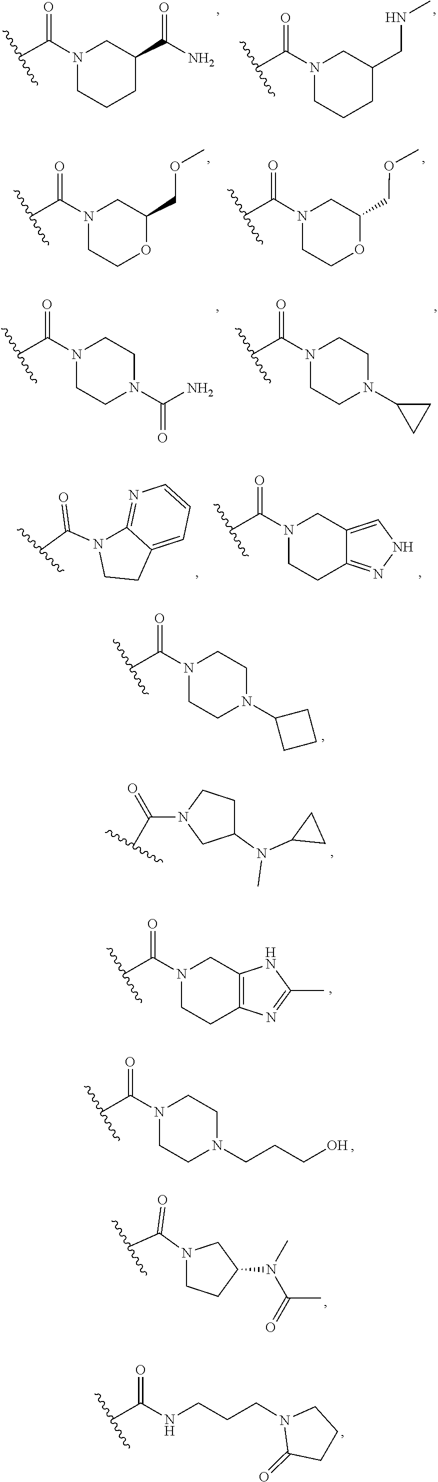

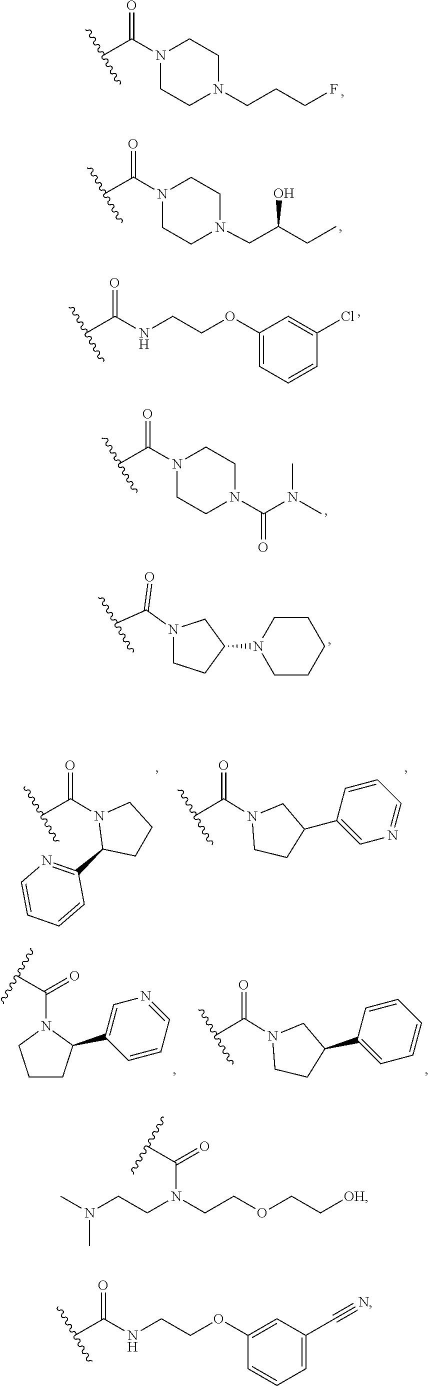

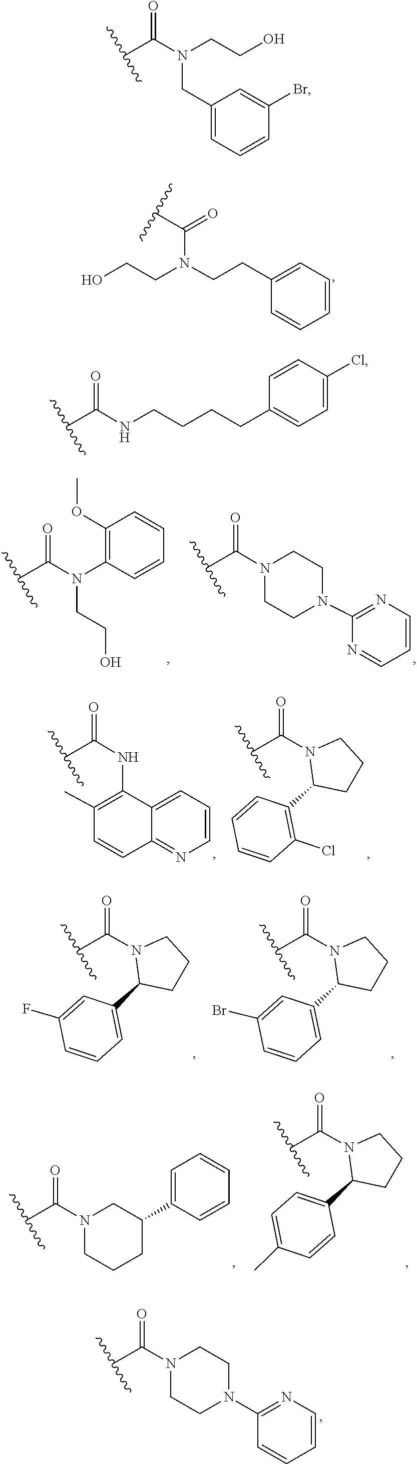

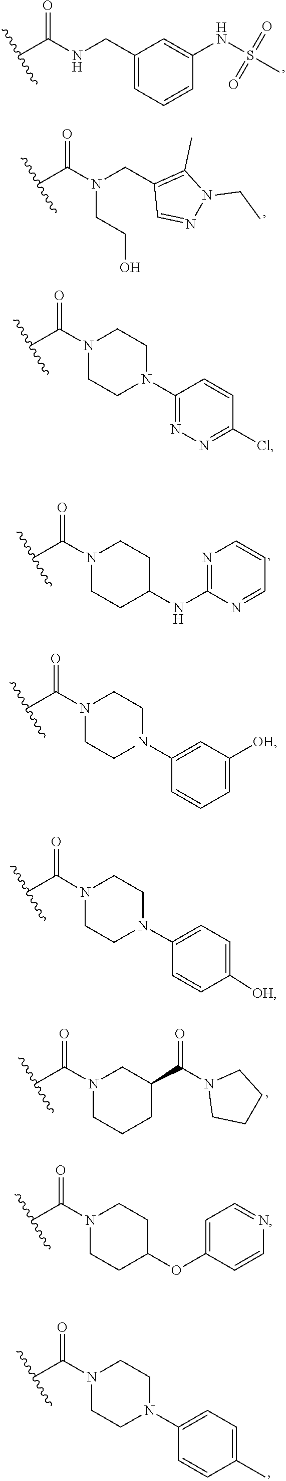

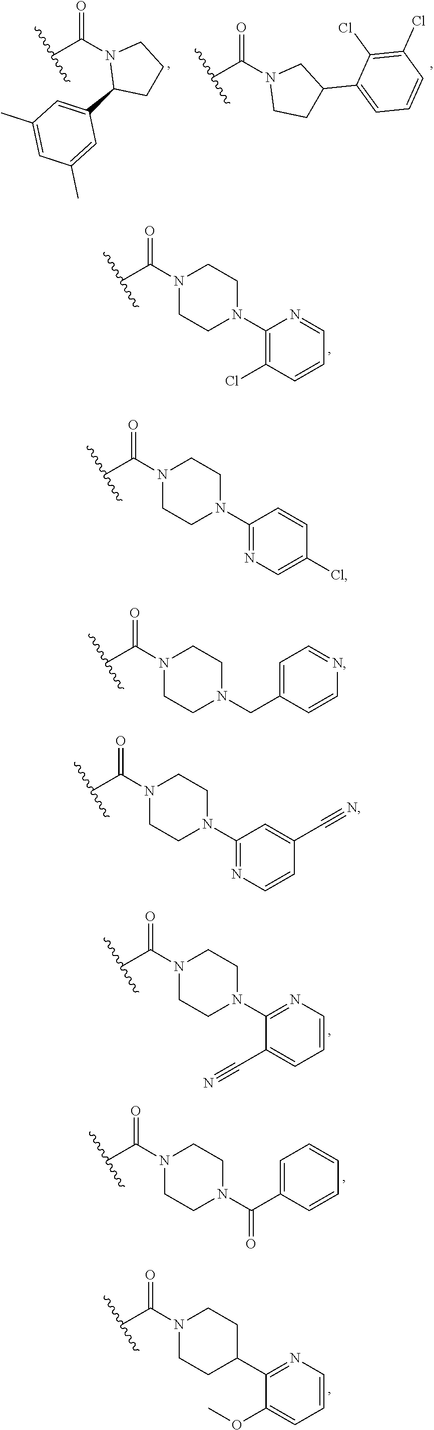

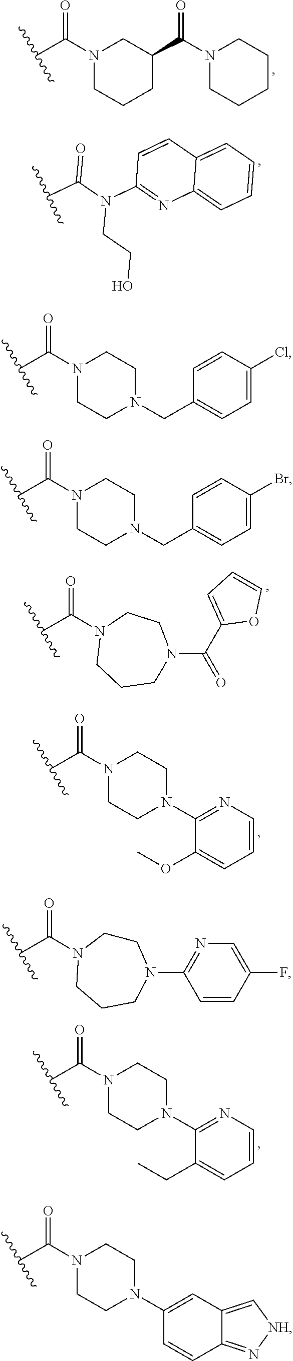

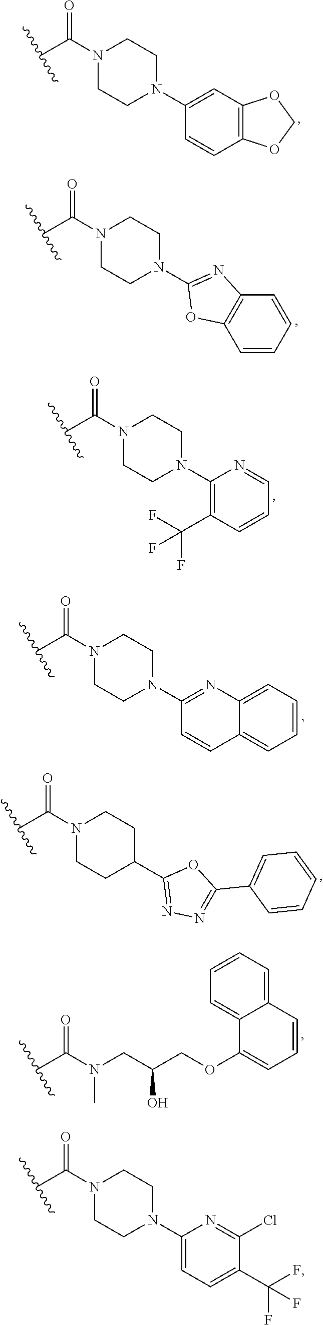

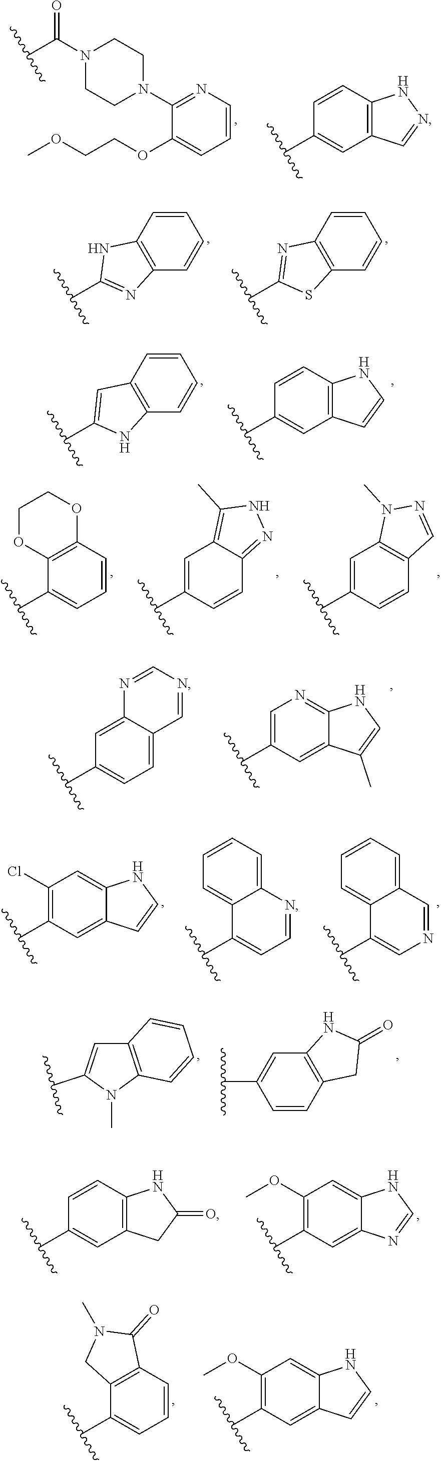

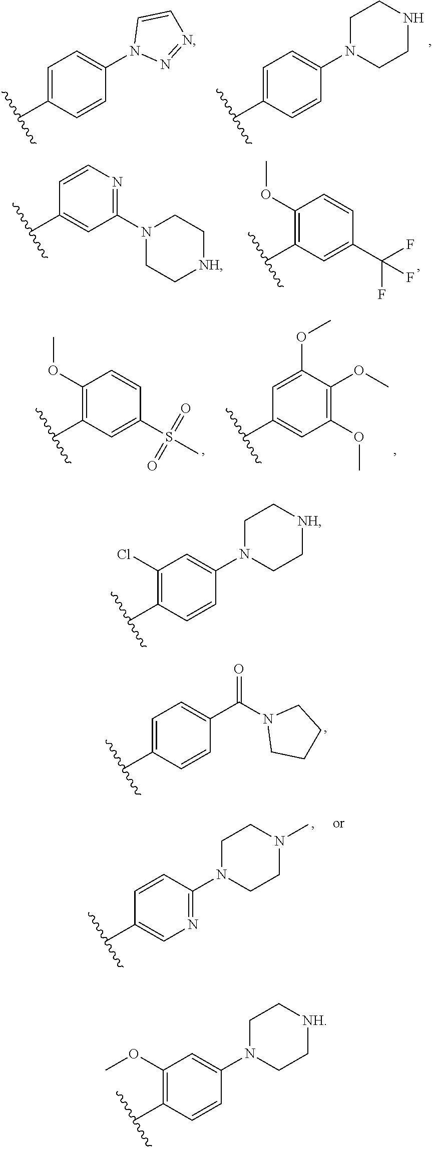

In some embodiments, R^{w} is

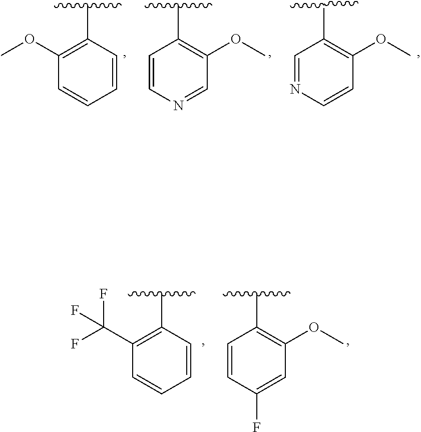

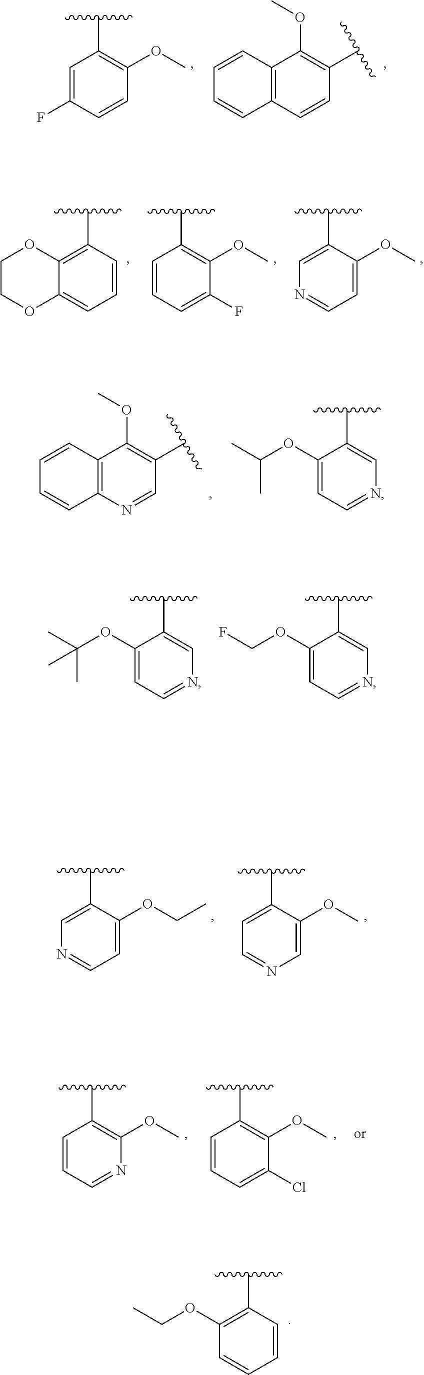

In some embodiments, R^{w} is S(O)₂NH₂, —S(O)₂N(CH₃)₂, —S(O)(NH)CH₃,

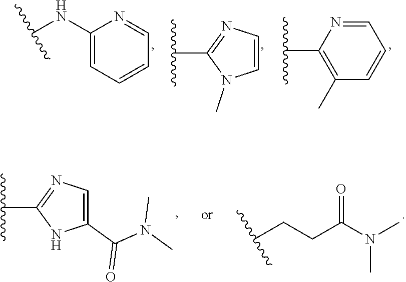

In some embodiments, the present invention provides a compound of any one of the following formulae:

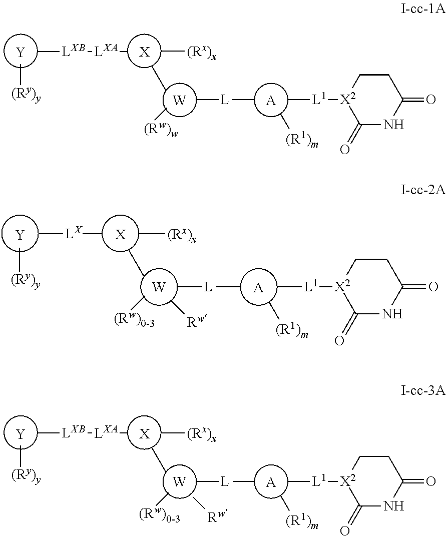

or a pharmaceutically acceptable salt thereof, wherein each variable is as defined above and described herein both individually and in combination.

In some embodiments, the present invention provides a compound of any one of the following formulae:

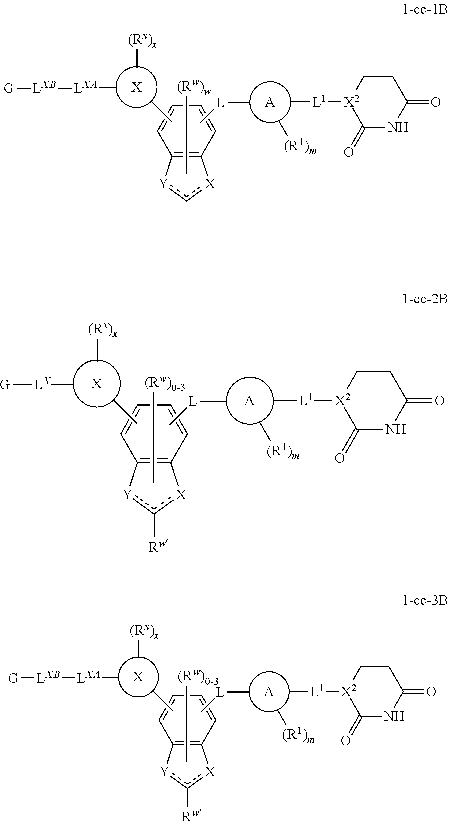

or a pharmaceutically acceptable salt thereof, wherein each variable is as defined above and described herein both individually and in combination.

In some embodiments, the present invention provides a compound of any one of the following formulae:

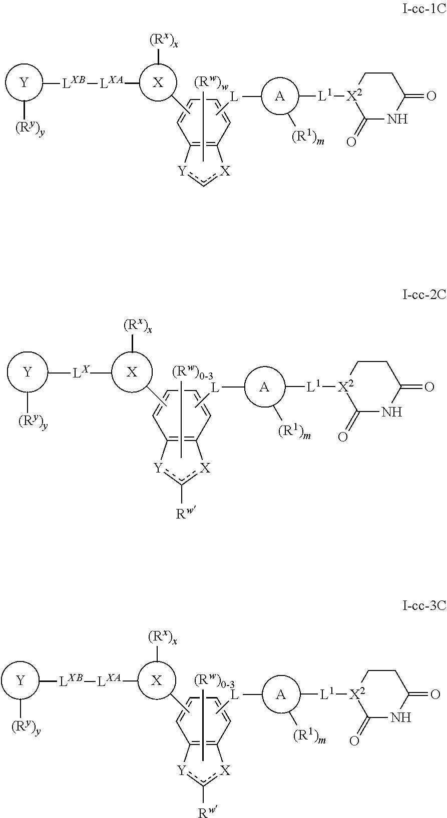

or a pharmaceutically acceptable salt thereof, wherein each variable is as defined above and described herein both individually and in combination.

In some embodiments, the present invention provides a compound of any one of the following formulae:

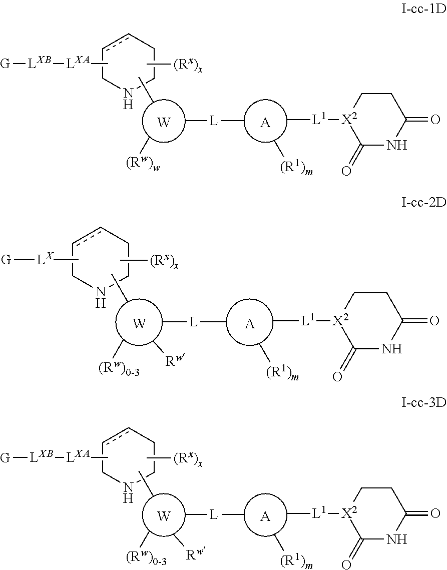

or a pharmaceutically acceptable salt thereof, wherein each variable is as defined above and described herein both individually and in combination.

In some embodiments, the present invention provides a compound of any one of the

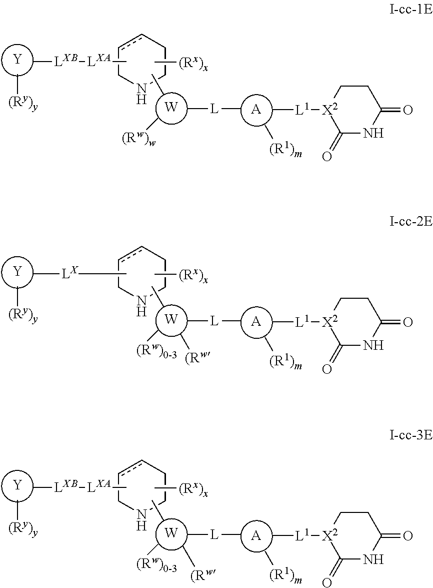

or a pharmaceutically acceptable salt thereof, wherein each variable is as defined above and described herein both individually and in combination.

In some embodiments, the present invention provides a compound of any one of the following formulae:

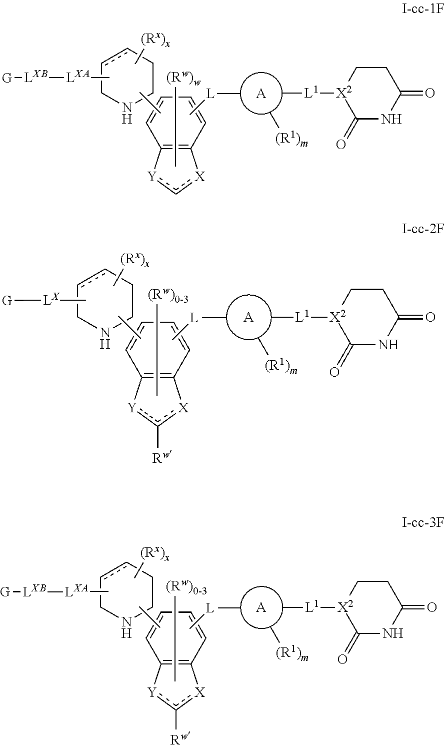

or a pharmaceutically acceptable salt thereof, wherein each variable is as defined above and described herein both individually and in combination.

In some embodiments, the present invention provides a compound of any one of the following formulae:

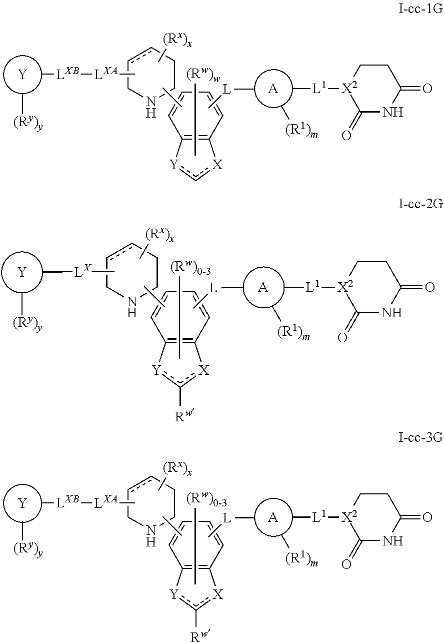

or a pharmaceutically acceptable salt thereof, wherein each variable is as defined above and described herein both individually and in combination.

In some embodiments, the present invention provides a compound of any one of the following formulae:

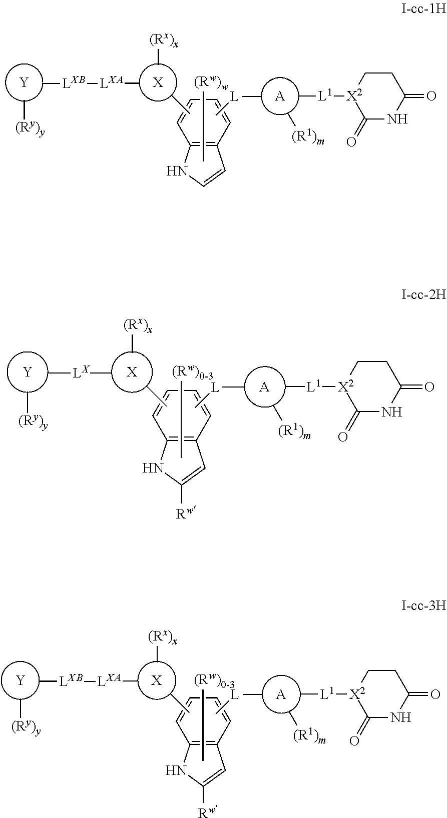

or a pharmaceutically acceptable salt thereof, wherein each variable is as defined above and described herein both individually and in combination.

In some embodiments, the present invention provides a compound of any one of the

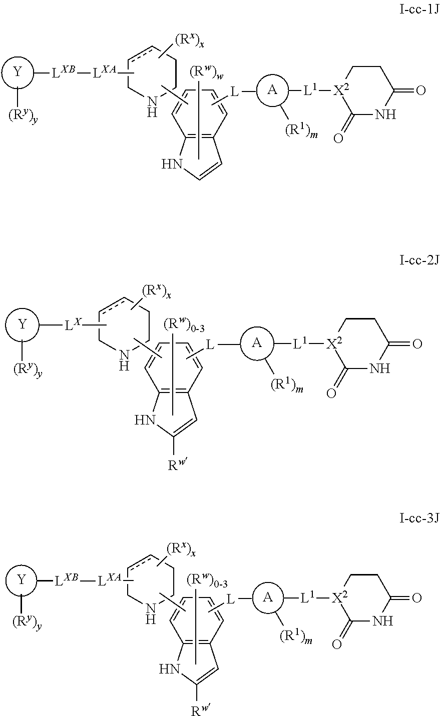

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A, I-cc-1B, I-cc-2B, I-cc-3B, I-cc-1C, I-cc-2C, I-cc-3C, I-cc-1D, I-cc-2D, I-cc-3D, I-cc-1E, I-cc-2E, I-cc-3E, I-cc-1F, I-cc-2F, I-cc-3F, I-cc-1G, I-cc-2G, I-cc-3G, I-cc-1H, I-cc-2H, I-cc-3H, I-cc-1J, I-cc-2J, or I-cc-3J, the structure

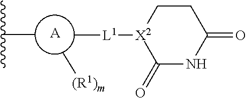

is of any one of formula I-aa-10′, I-aa-11′, I-aa-12′, I-aa-13′, or I-aa-14′:

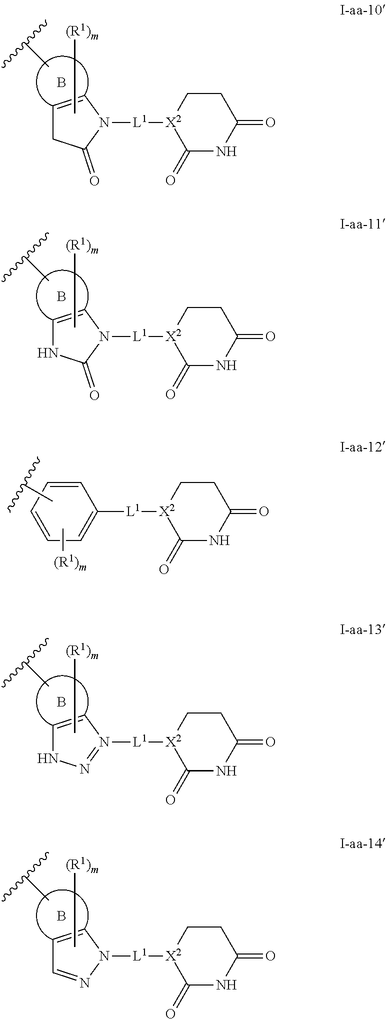

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A, I-cc-1B, I-cc-2B, I-cc-3B, I-cc-1C, I-cc-2C, I-cc-3C, I-cc-1D, I-cc-2D, I-cc-3D, I-cc-1E, I-cc-2E, I-cc-3E, I-cc-1F, I-cc-2F, I-cc-3F, I-cc-1G, I-cc-2G, I-cc-3G, I-cc-1H, I-cc-2H, I-cc-3H, I-cc-1J, I-cc-2J, or I-cc-3J, the structure

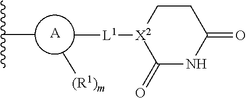

is of any one of formula I-aa-10a′, I-aa-11a′, I-aa-12a′, I-aa-12b′, I-aa-13a′, or I-aa-14a′:

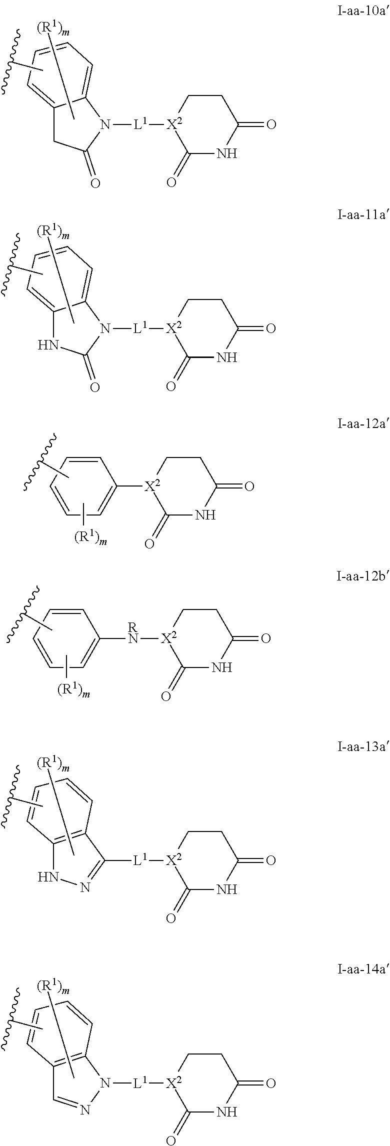

or a pharmaceutically acceptable salt thereof, wherein each variable is as defined above and described herein both individually and in combination.

In some embodiments, the present invention provides a compound of any one of the following formulae:

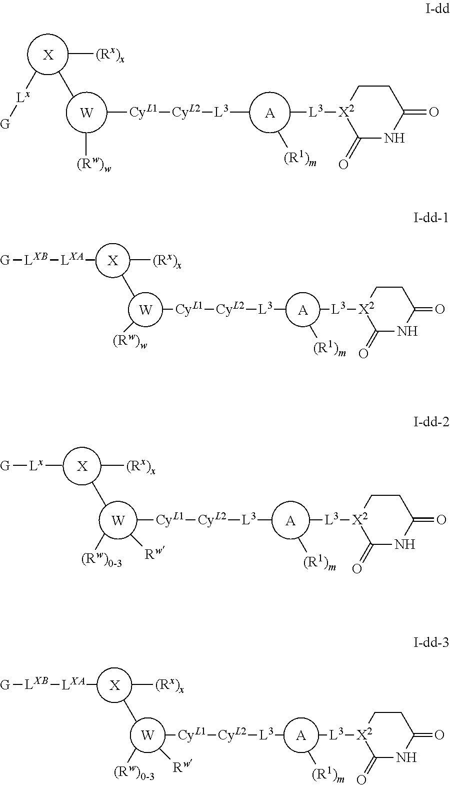

or a pharmaceutically acceptable salt thereof, wherein each variable is as defined above and described herein both individually and in combination.

In some embodiments, the present invention provides a compound of any one of the following formulae:

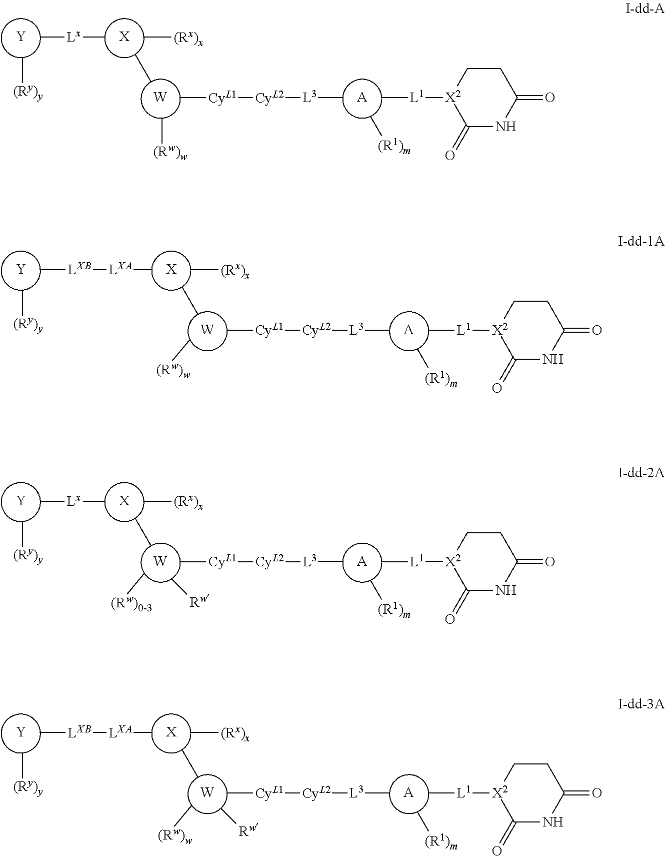

or a pharmaceutically acceptable salt thereof, wherein each variable is as defined above and described herein both individually and in combination.

In some embodiments, the present invention provides a compound of any one of the following formulae:

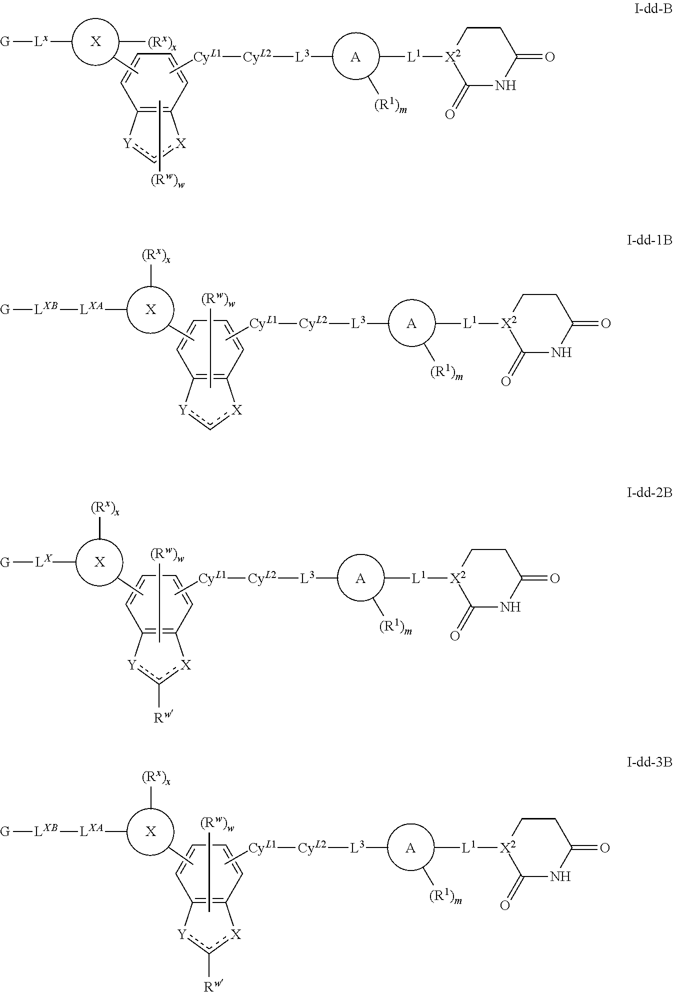

or a pharmaceutically acceptable salt thereof, wherein each variable is as defined above and described herein both individually and in combination.

In some embodiments, the present invention provides a compound of any one of the following formulae:

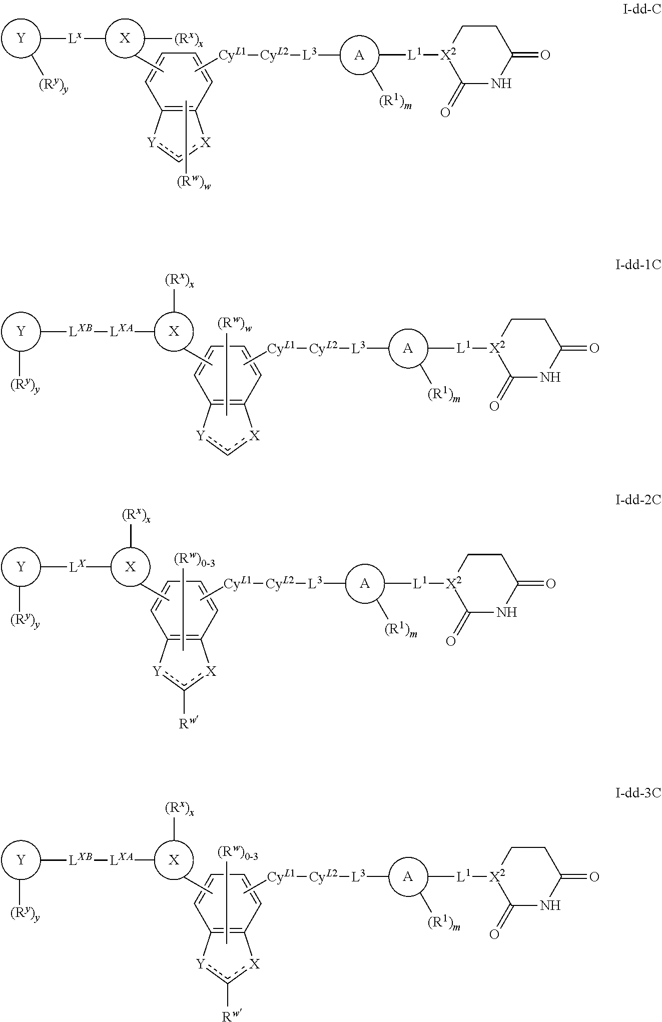

or a pharmaceutically acceptable salt thereof, wherein each variable is as defined above and described herein both individually and in combination.

In some embodiments, the present invention provides a compound of any one of the following formulae:

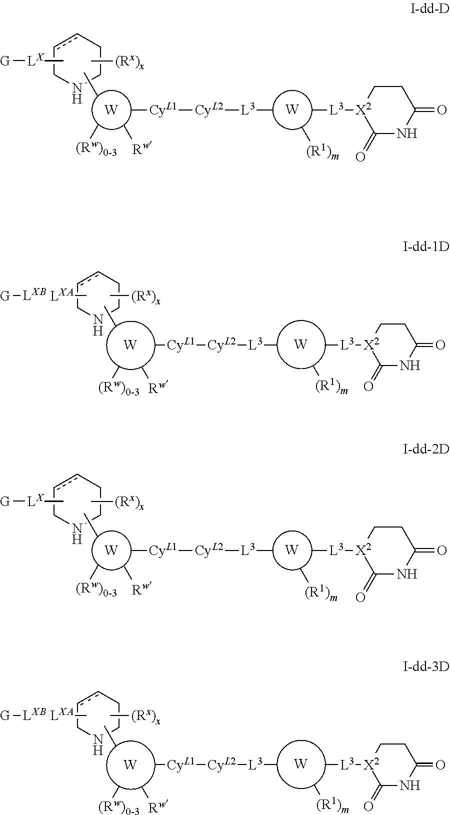

or a pharmaceutically acceptable salt thereof, wherein each variable is as defined above and described herein both individually and in combination.

In some embodiments, the present invention provides a compound of any one of the following formulae:

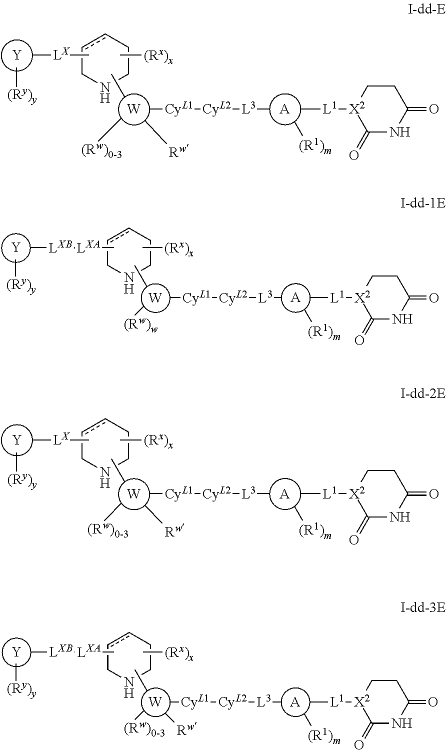

or a pharmaceutically acceptable salt thereof, wherein each variable is as defined above and described herein both individually and in combination.

In some embodiments, the present invention provides a compound of any one of the following formulae:

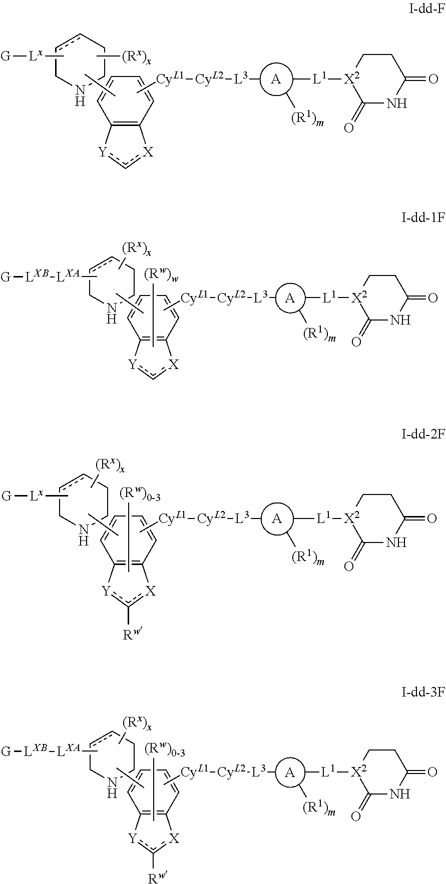

or a pharmaceutically acceptable salt thereof, wherein each variable is as defined above and described herein both individually and in combination.

In some embodiments, the present invention provides a compound of any one of the following formulae:

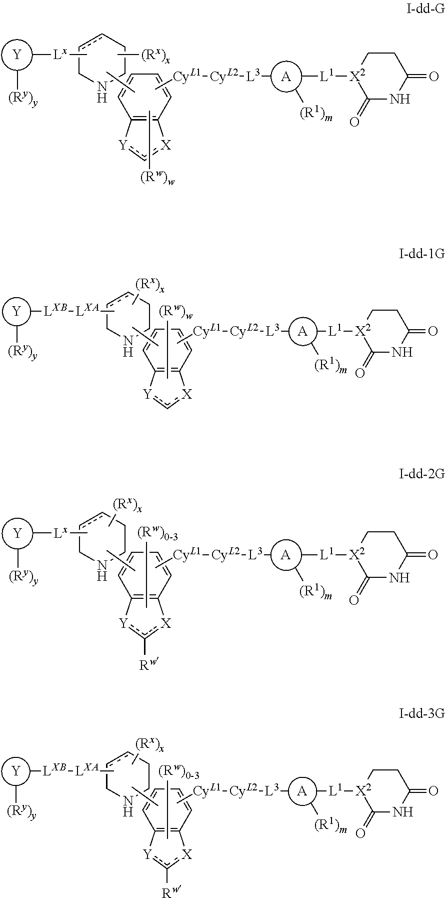

or a pharmaceutically acceptable salt thereof, wherein each variable is as defined above and described herein both individually and in combination.

In some embodiments, the present invention provides a compound of any one of the following formulae:

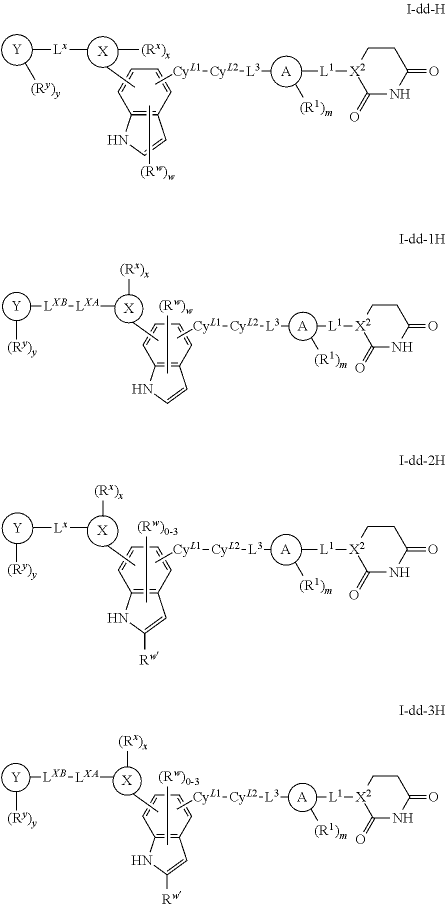

or a pharmaceutically acceptable salt thereof, wherein each variable is as defined above and described herein both individually and in combination.

In some embodiments, the present invention provides a compound of any one of the following formulae:

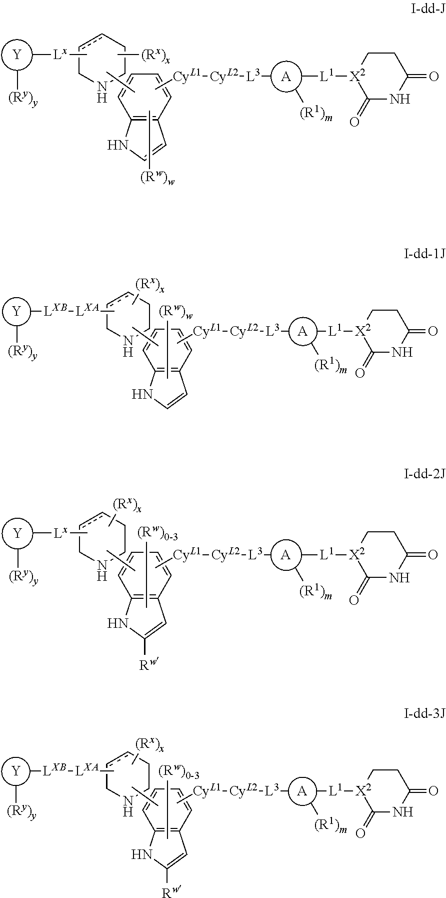

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B, I-dd-C, I-dd-1C, I-dd-2C, I-dd-3C, I-dd-D, I-dd-1D, I-dd-2D, I-dd-3D, I-dd-E, I-dd-1E, I-dd-2E, I-dd-3E, I-dd-F, I-dd-1F, I-dd-2F, I-dd-3F, I-dd-G, I-dd-1G, I-dd-2G, I-dd-3G, I-dd-H, I-dd-1H, I-dd-2H, I-dd-3H, I-dd-J, I-dd-1J, I-dd-2J, or I-dd-3J, the structure

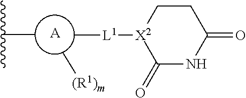

is of any one of formula I-aa-10′, I-aa-11′, I-aa-12′, I-aa-13′, or I-aa-14′:

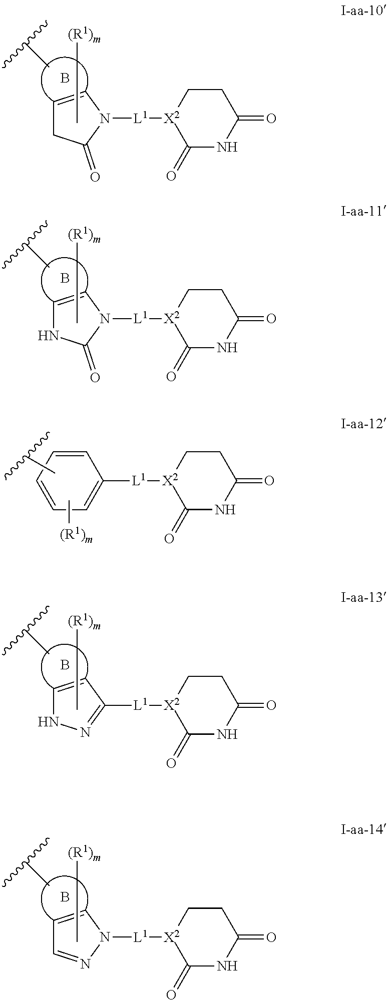

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B, I-dd-C, I-dd-1C, I-dd-2C, I-dd-3C, I-dd-D, I-dd-1D, I-dd-2D, I-dd-3D, I-dd-E, I-dd-1E, I-dd-2E, I-dd-3E, I-dd-F, I-dd-1F, I-dd-2F, I-dd-3F, I-dd-G, I-dd-1G, I-dd-G, I-dd-G, I-dd-H H, I-dd-1, I-dd-H, I-dd-H, I-dd-J, I-dd-2J, I-dd-, or I-dd-3J, the structure

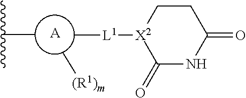

is of any one of formula I-aa-10a′, I-aa-11a′, I-aa-12a′, I-aa-12b′, I-aa-13a′, or I-aa-14a′:

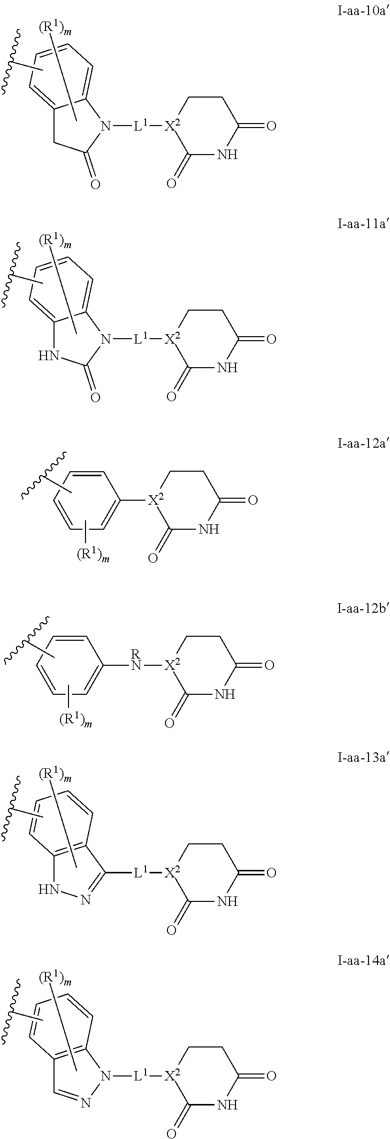

or a pharmaceutically acceptable salt thereof, wherein each variable is as defined above and described herein both individually and in combination.

Exemplary compounds of the invention are set forth in Table 1B below. It is understood that certain compounds herein may contain arbitrarily assigned stereochemistry or be derived from an intermediate with arbitrarily assigned stereochemistry. Unless otherwise stated, stereochemistry for compounds herein has been assigned arbitrarily, and it will be understood that any compound with arbitrarily assigned stereochemistry or produced from an intermediate with arbitrarily assigned stereochemistry may be depicted herein as a certain stereoisomer, but such compound may be the other stereoisomer (i.e., enantiomer or diastereomer). As shown in Table 1B, column “EO”, for instances where a pair of stereoisomers were produced and the final compounds separated, the order of elution is provided: the first eluting isomer is indicated by “E1” and the second elution isomer is indicated by “E2”. As shown in Table 1B, column “EO”, for instances where a pair of stereoisomers were produced, but a pair of intermediate stereoisomers were separated and further modified to arrive at the final compounds, the order of elution of the intermediate compound is provided: the first eluting isomer of the intermediate compound is indicated by “I1”, and the second eluting isomer of the intermediate compound is indicted by “I2.”

TABLE 1B
Exemplary Compounds

| I- | Structure |
|---|---|
| 61 | 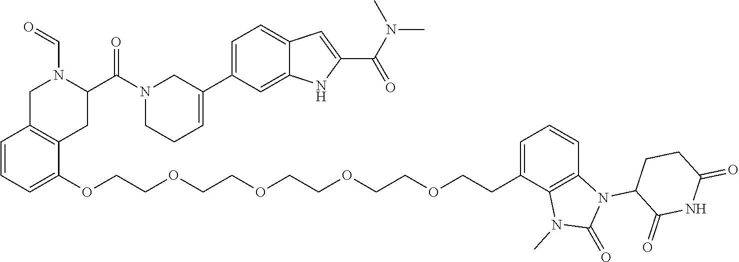 [SMILES: CN(C)C(=O)c1cc2ccc(C3=CCCN(C(=O)C4Cc5c(cccc5OCCOCCOCCOCCOCCc5cccc6c5n(C)c(=O)n6C5CCC(=O)NC5=O)CN4C=O)C3)cc2[nH]1] |
| 98 | 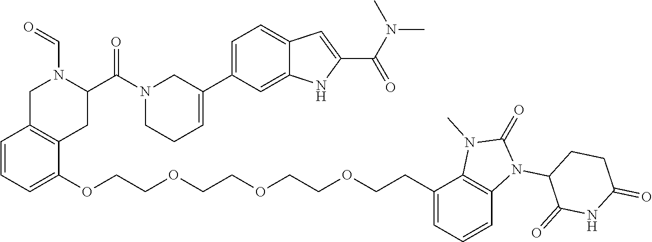 [SMILES: CN(C)C(=O)c1cc2ccc(C3=CCCN(C(=O)C4Cc5c(cccc5OCCOCCOCCOCCc5cccc6c5n(C)c(=O)n6C5CCC(=O)NC5=O)CN4C=O)C3)cc2[nH]1] |
| 105 | 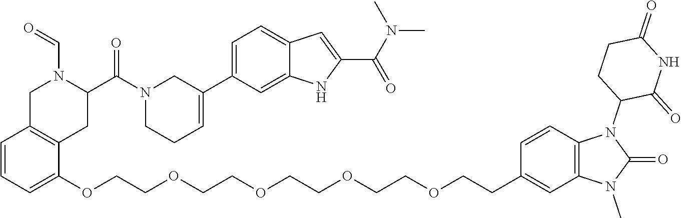 [SMILES: CN(C)C(=O)c1cc2ccc(C3=CCCN(C(=O)C4Cc5c(cccc5OCCOCCOCCOCCOCCc5ccc6c(c5)n(C)c(=O)n6C5CCC(=O)NC5=O)CN4C=O)C3)cc2[nH]1] |
| 1270 | 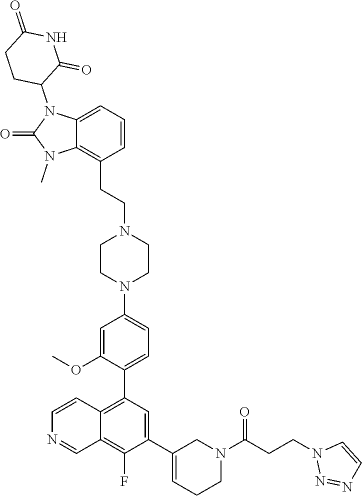 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2cnccc12] |
| 1271 | 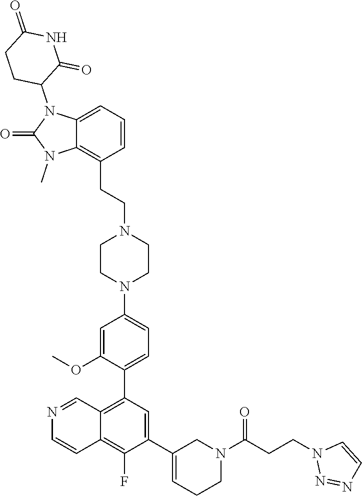 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2ccncc12] |
| 1272 | 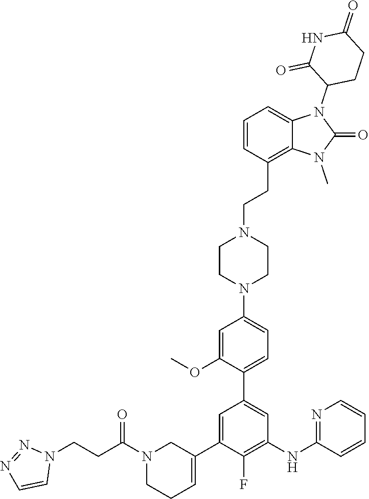 [SMILES: COc1cc(N2CCN(CCc3cccc4c3n(C)c(=O)n4C3CCC(=O)NC3=O)CC2)ccc1-c1cc(Nc2ccccn2)c(F)c(C2=CCCN(C(=O)CCn3ccnn3)C2)c1] |
| 1282 | 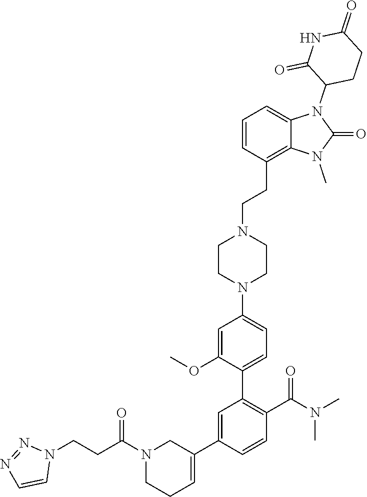 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1C(=O)N(C)C] |
| 1292 | 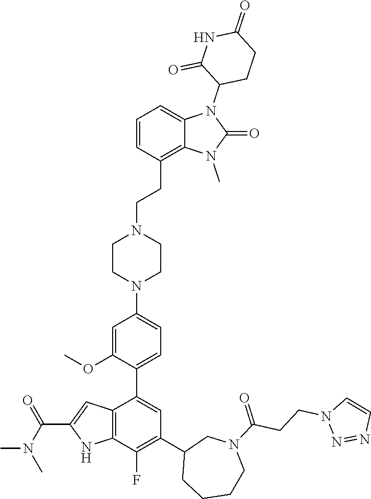 [SMILES: COc1cc(N2CCN(CCc3cccc4c3n(C)c(=O)n4C3CCC(=O)NC3=O)CC2)ccc1-c1cc(C2CCCCN(C(=O)CCn3ccnn3)C2)c(F)c2[nH]c(C(=O)N(C)C)cc12] |
| 1293 | 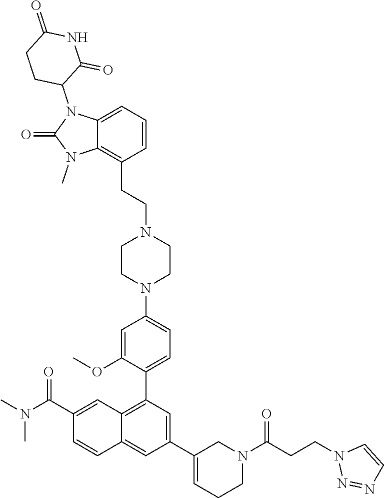 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccc(C(=O)N(C)C)cc12] |
| 1302 | 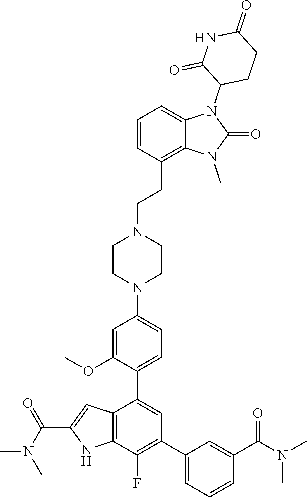 |
| 1309 | 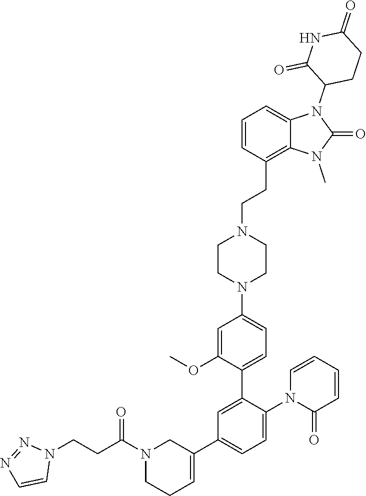 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-n1ccccc1=O] |
| 1323 | 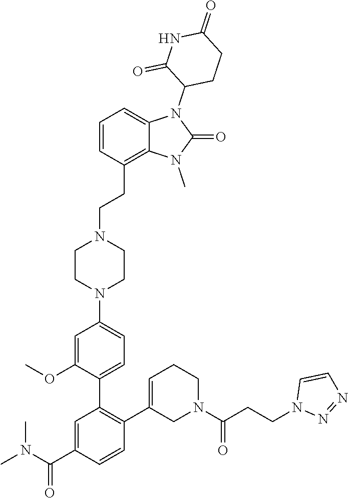 [SMILES: COc1cc(N2CCN(CCc3cccc4c3n(C)c(=O)n4C3CCC(=O)NC3=O)CC2)ccc1-c1cc(C(=O)N(C)C)ccc1C1=CCCN(C(=O)CCn2ccnn2)C1] |
| 1324 | 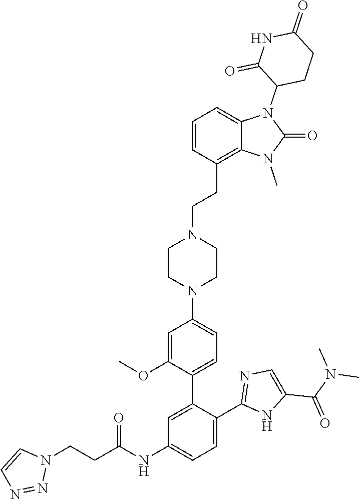 [SMILES: COc1cc(N2CCN(CCc3cccc4c3n(C)c(=O)n4C3CCC(=O)NC3=O)CC2)ccc1-c1cc(NC(=O)CCn2ccnn2)ccc1-c1ncc(C(=O)N(C)C)[nH]1] |
| 1325 | 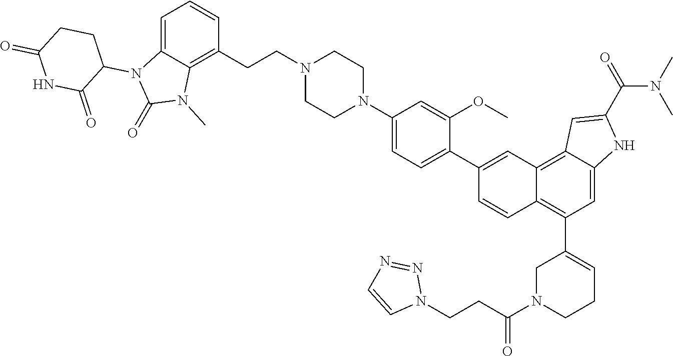 [SMILES: COc1cc(N2CCN(CCc3cccc4c3n(C)c(=O)n4C3CCC(=O)NC3=O)CC2)ccc1-c1ccc2c(C3=CCCN(C(=O)CCn4ccnn4)C3)cc3[nH]c(C(=O)N(C)C)cc3c2c1] |
| 1326 | 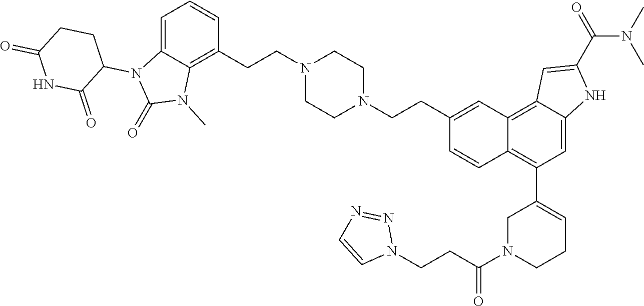 [SMILES: CN(C)C(=O)c1cc2c(cc(C3=CCCN(C(=O)CCn4ccnn4)C3)c3ccc(CCN4CCN(CCc5cccc6c5n(C)c(=O)n6C5CCC(=O)NC5=O)CC4)cc32)[nH]1] |
| 1332 | 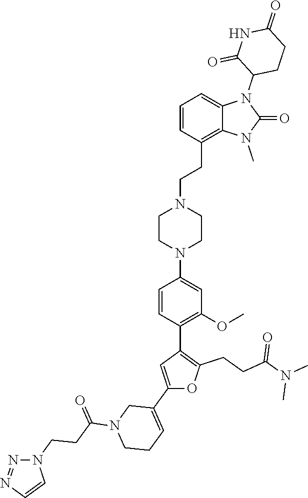 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)oc1CCC(=O)N(C)C] |
| 1333 | 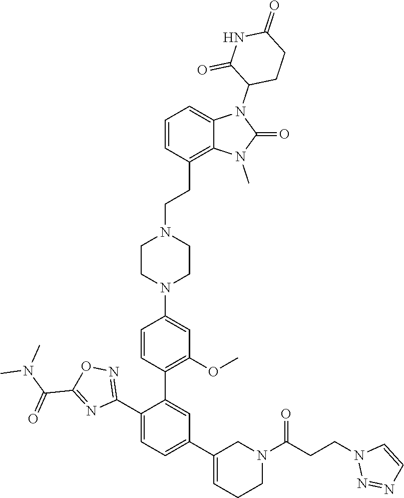 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-c1noc(C(=O)N(C)C)n1] |
| 1334 | 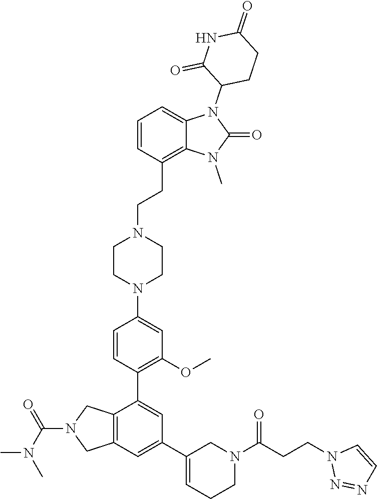 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1CN(C(=O)N(C)C)C2] |
| 1336 | 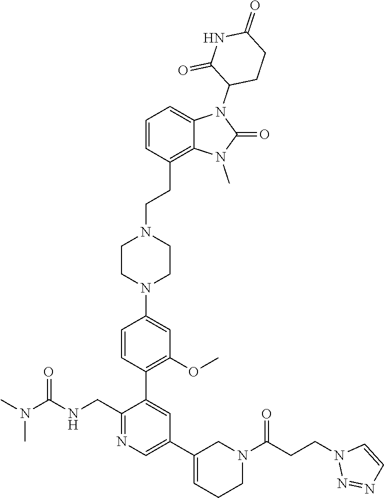 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cnc1CNC(=O)N(C)C] |
| 1338 | 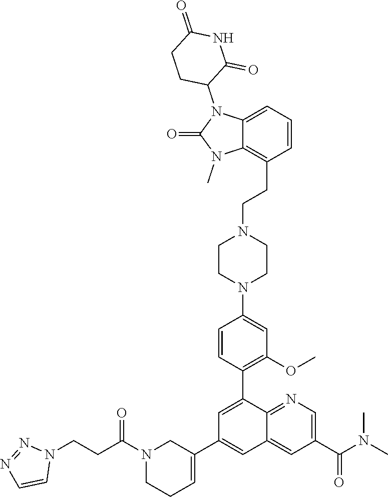 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2cc(C(=O)N(C)C)cnc12] |
| 1340 | 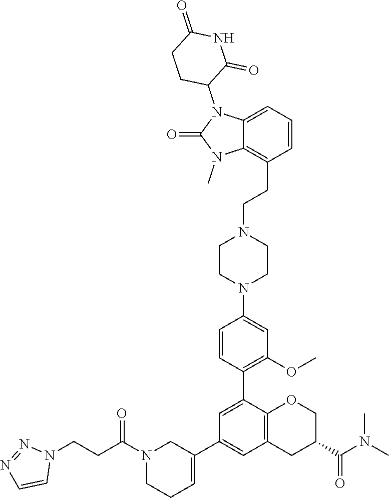 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1OC[C@H](C(=O)N(C)C)C2] |
| 1342 | 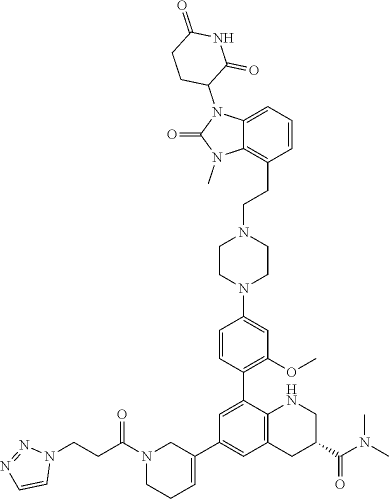 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1NC[C@H](C(=O)N(C)C)C2] |
| 1401 | 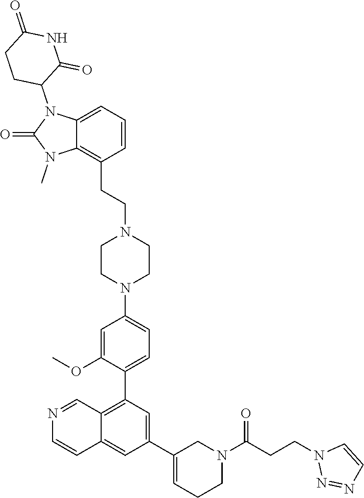 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccncc12] |

In some embodiments, the present invention provides a compound set forth in Table 1B, above, or a pharmaceutically acceptable salt thereof. In some embodiments, the present invention provides a compound set forth in Table 1B, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, adjuvant, or vehicle. For example, in some embodiments, the present invention provides a pharmaceutical composition comprising a compound of formula I as defined above, or a pharmaceutically acceptable salt thereof, together with a pharmaceutically acceptable carrier, adjuvant, or vehicle. In some embodiments, the present invention provides a pharmaceutical composition comprising a compound of formula I as defined above, together with a pharmaceutically acceptable carrier, adjuvant, or vehicle. In some embodiments, the present invention provides a pharmaceutical composition comprising a compound set forth in Table 1B above, or a pharmaceutically acceptable salt thereof, together with a pharmaceutically acceptable carrier, adjuvant, or vehicle. In some embodiments, the present invention provides a pharmaceutical composition comprising a compound set forth in Table 1B above, together with a pharmaceutically acceptable carrier, adjuvant, or vehicle.

In some embodiments, the present invention provides a compound described herein (such as a compound of formula I as defined above), or a pharmaceutically acceptable salt thereof, or a pharmaceutical composition comprising a compound described herein (such as a compound of formula I as defined above), or a pharmaceutically acceptable salt thereof, and a pharmaceutically acceptable carrier, adjuvant, or vehicle for use as a medicament.

In some embodiments, the invention also provides a compound described herein (such as a compound of formula I), or pharmaceutical compositions described herein, for use in a method for degrading STAT6 as described herein and/or in a method for treating a STAT6-mediated disorder as described herein. In some embodiments, the invention also provides a compound described herein (such as a compound of formula I), or pharmaceutical compositions described herein, for use in a method for degrading STAT6 as described herein. In some embodiments, the invention also provides a compound described herein (such as a compound of formula I), or pharmaceutical compositions described herein, for use in a method for treating a STAT6-mediated disorder as described herein.

Another aspect of the present disclosure relates to a method of treating a STAT6-mediated disorder, disease, or condition in a subject in need thereof, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of treating a STAT6-mediated disorder, disease, or condition in a subject in need thereof, wherein the method comprises administering to said subject a compound disclosed herein, or a pharmaceutical acceptable salt thereof: and wherein the method results in the subject having a reduced STAT6 level relative to an untreated subject with the same STAT6-mediated disorder, disease, or condition.

In some embodiments, the STAT6 is from a sample. In some embodiments, a sample comprises a biological sample. In some embodiments, the biological sample is taken from the subject. In some embodiments, the sample comprises a lysate. In some embodiments, the sample comprises a peripheral blood mononuclear cell (PBMC).

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to an untreated subject with the same STAT6-mediated disorder, disease, or condition.

In some embodiments, the STAT6 is from a sample. In some embodiments, the sample comprises a biological sample. In some embodiments, the biological sample is taken from the subject. In some embodiments, the sample comprises a lysate. In some embodiments, the sample comprises a peripheral blood mononuclear cell (PBMC). In some embodiments, the STAT6 is a polypeptide disclosed herein. In some embodiments, the STAT6 is a polypeptide of a plurality of polypeptides disclosed herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In certain embodiments, compounds of the present invention are generally prepared according to any one of the schemes set forth below:

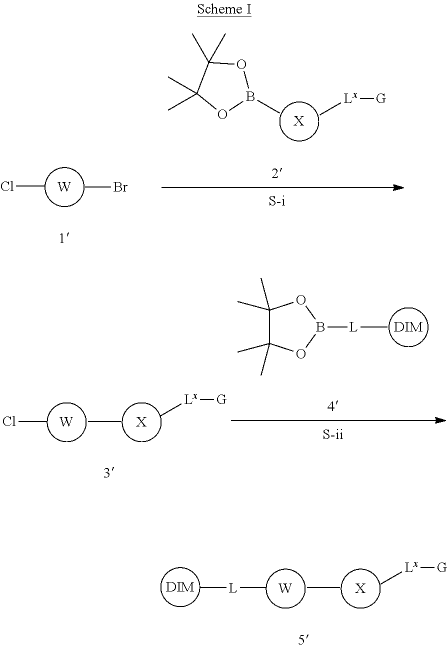

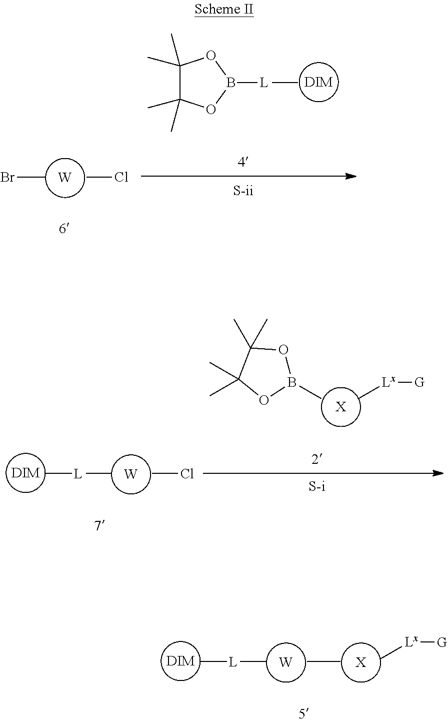

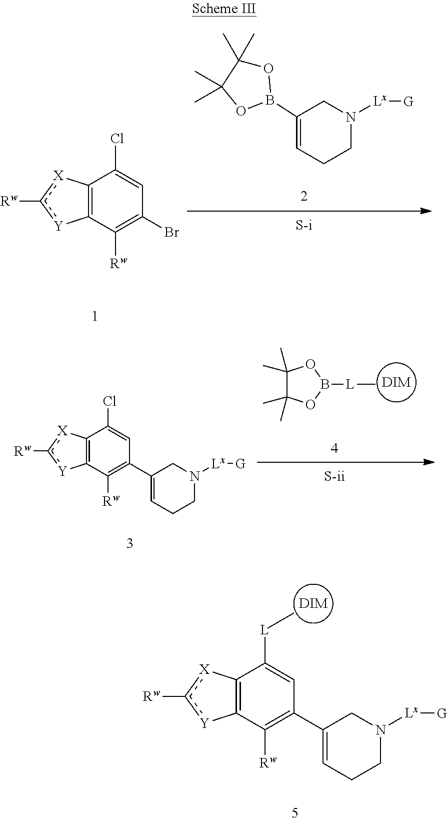

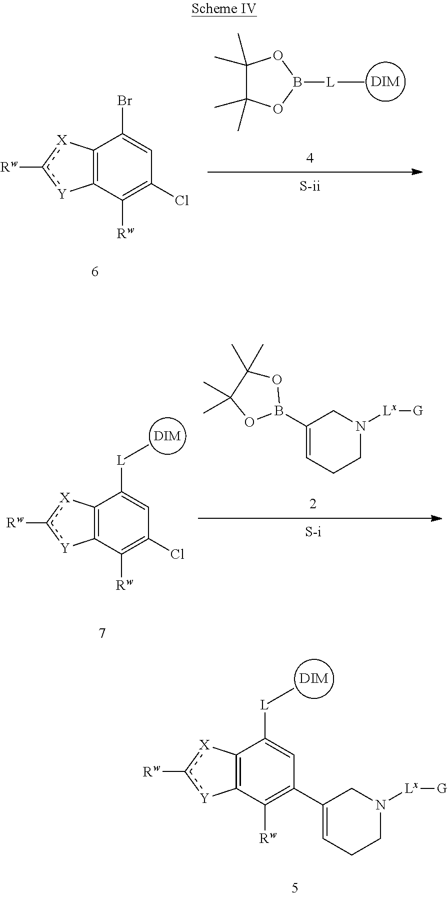

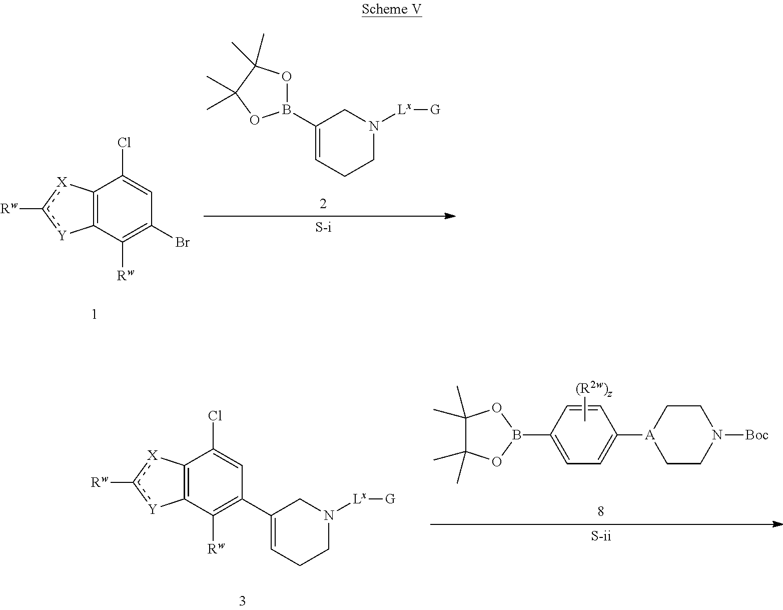

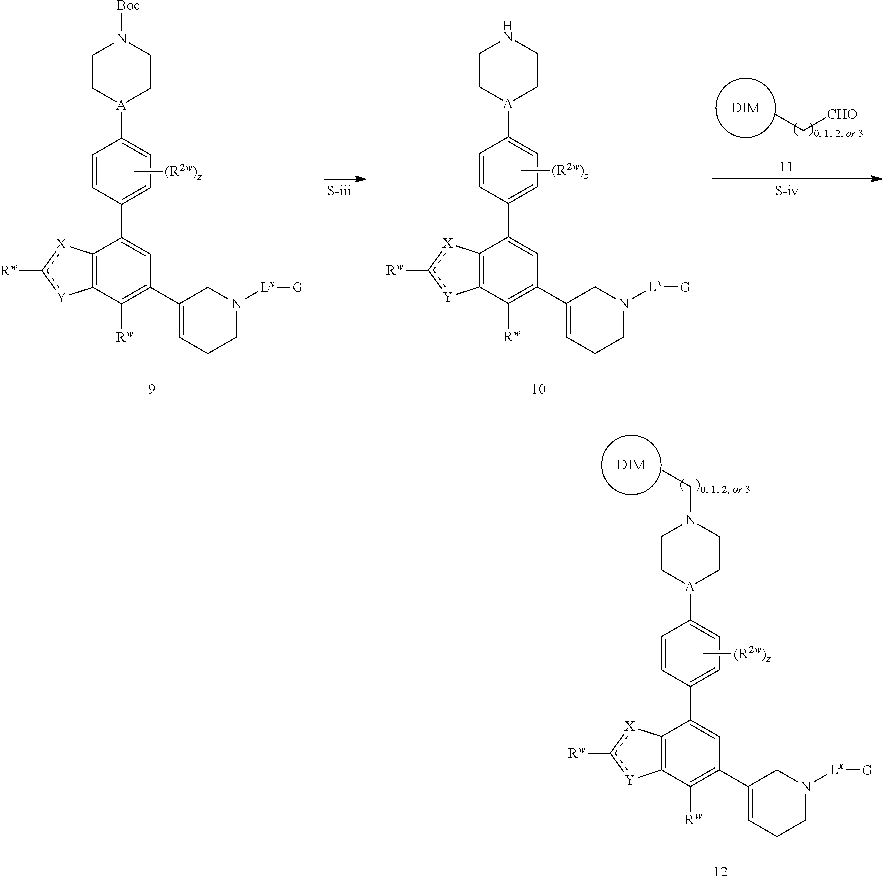

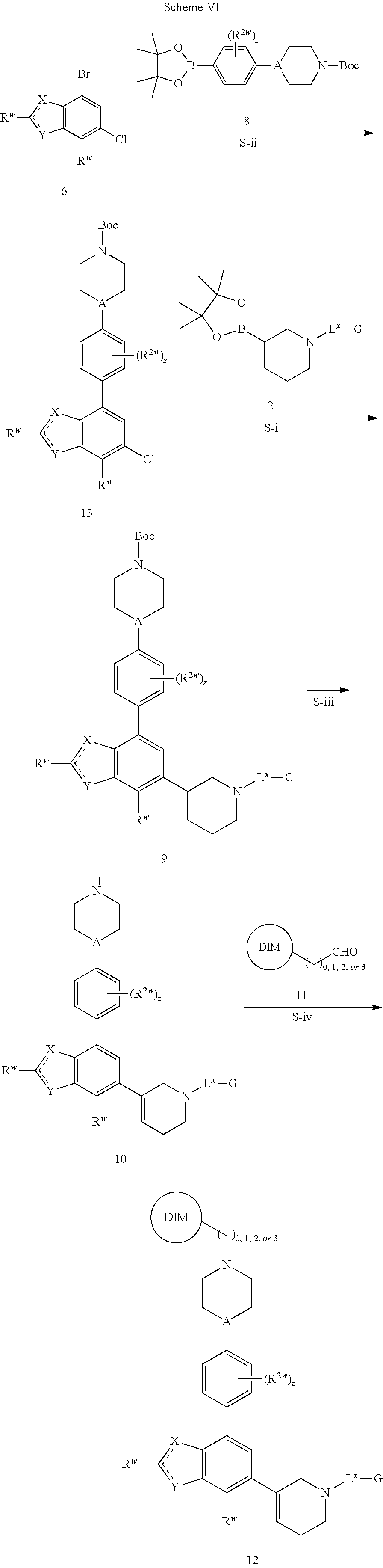

In the schemes above, each of Ring W. Ring X, X, Y, W, R^{2w}, L^{x}, G, and z is as defined above and in classes and subclasses as described herein.

In one aspect, the present invention provides methods for preparing certain compounds of formula I according to the steps depicted in Scheme I-VI above. In some embodiments, step S-i comprises contacting a compound of formula 1, 1′, 7, 7′, or 13 with a compound of formula 2 or 2′ in the presence of a palladium catalyst and a base in a solvent. In some embodiments, the palladium catalyst includes a phosphine ligand (e.g., dppf). In some embodiments, the base is a carbonate (e.g., K₂CO₃ or Cs₂CO₃) or a phosphate (e.g., K₃PO₄). In some embodiments, the solvent is an aqueous mixture including a polar aprotic solvent (e.g., dioxane). In some embodiments, step S-i comprises the compounds, reagents, and conditions described in the below Examples section.

In some embodiments, step S-ii comprises contacting a compound of formula 3, 3′, 6 or 6′ with a compound of formula 4 or 4′ in the presence of a palladium catalyst and a base in a solvent. In some embodiments, the palladium catalyst includes a phosphine ligand (e.g., Xphos). In some embodiments, the base is a carbonate (e.g., K₂CO₃ or Cs₂CO₃) or a phosphate (e.g., K₃PO₄). In some embodiments, the solvent is an aqueous mixture including a polar aprotic solvent (e.g., dioxane). In some embodiments, step S-ii comprises the compounds, reagents, and conditions described in the below Examples section.

In some embodiments, step S-iii comprises contacting a compound of formula 9 with an acid in a solvent. In some embodiments, the acid is an inorganic base (e.g., HCl) or organic base (e.g., TFA). In some embodiments, the solvent is a polar aprotic solvent (e.g., DCM). In some embodiments, step S-iii comprises the compounds, reagents, and conditions described in the below Examples section.

In some embodiments, step S-iv comprises contacting a compound of formula 10 with a compound of formula 11 in the presence of an reducing agent in a solvent. In some embodiments, the reducing agent is a borohydride compound (e.g., sodium cyanoborohydride or sodium triacetoxyborohydride). In some embodiments, the solvent is a polar aprotic solvent (e.g., DMSO). In some embodiments, step S-iv comprises the compounds, reagents, and conditions described in the below Examples section.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit STAT6 protein, or a mutant thereof, in a biological sample or in a patient. In certain embodiments, the amount of compound in compositions of this invention is such that is effective to measurably degrade and/or inhibit STAT6 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitory active metabolite or residue thereof” means that a metabolite or residue thereof is also an inhibitor of STAT6 protein, or a mutant thereof.

As used herein, the term “degradatory active metabolite or residue thereof” means that a metabolite or residue thereof is also a degrader of STAT6 protein, or a mutant thereof.

In certain embodiments, a provided compound is administered as a prodrug.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of STAT6 protein activity.

According to one embodiment, the invention relates to a method of inhibiting or degrading STAT6 or a mutant thereof, activity in a biological sample comprising the step of contacting said biological sample with a compound of this invention, or a composition comprising said compound.

The term “biological sample”, as used herein, includes, without limitation, cell cultures or extracts thereof: biopsied material obtained from a mammal or extracts thereof; and blood, saliva, urine, feces, semen, tears, or other body fluids or extracts thereof. In some embodiments, the STAT6 is from a biological sample. In some embodiments, the biological sample is taken from a subject.

Inhibition and/or degradation of STAT6, or a mutant thereof, activity in a biological sample is useful for a variety of purposes that are known to one of skill in the art. Examples of such purposes include, but are not limited to, blood transfusion, organ-transplantation, biological specimen storage, and biological assays.

According to another embodiment, the invention relates to a method of degrading and/or inhibiting STAT6, or a mutant thereof, activity in a patient comprising the step of administering to said patient a compound of the present invention, or a composition comprising said compound. In other embodiments, the present invention provides a method for treating a disorder mediated by STAT6 or a mutant thereof, in a patient in need thereof, comprising the step of administering to said patient a compound according to the present invention or pharmaceutically acceptable composition thereof. Such disorders are described in detail herein.

In some embodiments, the invention described herein provides a method of degrading STAT6 in a subject, wherein the method comprises administering to said subject a compound described herein, or a pharmaceutical acceptable salt thereof, and wherein the method results in the subject having a reduced STAT6 level relative to the subject prior to administering the compound described herein, or a pharmaceutical acceptable salt thereof. In some embodiments, the STAT6 is from a biological sample. In some embodiments, the biological sample is taken from the subject. In some embodiments, the STAT6 is the polypeptide described herein.

In some embodiments, the invention described herein provides a method of degrading STAT6 in a subject, wherein the method comprises administering to said subject a compound described herein, or a pharmaceutical acceptable salt thereof; and wherein the method results in the subject having a reduced STAT6 level relative to an untreated subject with the same STAT6-mediated disorder, disease, or condition. In some embodiments, the STAT6 is from a biological sample. In some embodiments, the biological sample is taken from the subject. In some embodiments, the STAT6 is the polypeptide described herein.

In some embodiments, the invention described herein provides a method of treating a STAT6-mediated disorder, disease, or condition in a subject in need thereof, wherein the method comprises administering to said subject a compound described herein, or a pharmaceutical acceptable salt thereof: and wherein the method results in the subject having a reduced STAT6 level relative to the subject prior to administering the compound described herein, or a pharmaceutical acceptable salt thereof. In some embodiments, the STAT6 is from a biological sample. In some embodiments, the biological sample is taken from the subject. In some embodiments, the STAT6 is the polypeptide described herein.

In some embodiments, described herein is a method of treating a STAT6-mediated disorder, disease, or condition in a subject in need thereof, wherein the method comprises administering to said subject a compound described herein, or a pharmaceutical acceptable salt thereof; and wherein the method results in the subject having a reduced STAT6 level relative to an untreated subject with the same STAT6-meditated disorder, disease, or condition. In some embodiments, the STAT6 is from a biological sample. In some embodiments, the biological sample is taken from the subject. In some embodiments, the STAT6 is the polypeptide described herein.

The activity of a compound utilized in this invention as a degrader and/or inhibitor of STAT6 or a mutant thereof, may be assayed in vitro, in vivo or in a cell line. In vitro assays include assays that determine inhibition of either the activity and/or the subsequent functional consequences of activated STAT6 protein or a mutant thereof. Alternate in vitro assays quantitate the ability of the inhibitor to bind to STAT6 protein. Inhibitor binding may be measured by radiolabeling the inhibitor prior to binding, isolating the inhibitor/STAT6 complex and determining the amount of radiolabel bound. Alternatively, inhibitor binding may be determined by running a competition experiment where new inhibitors are incubated with STAT6 protein bound to known radioligands. Detailed conditions for assaying a compound utilized in this invention as a degrader and/or inhibitor of STAT proteins, or a mutant thereof are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of STAT6 protein and are therefore useful for treating one or more disorders associated with activity of STAT6 protein. Thus, in certain embodiments, the present invention provides a method for treating a STAT6-mediated disorder comprising the step of administering to a patient in need thereof a compound of the present invention, or pharmaceutically acceptable composition thereof.

As used herein, the term “STAT6-mediated” disorders, diseases, and/or conditions as used herein means any disease or other deleterious condition in which STAT6 or a mutant thereof, are known to play a role. Accordingly, another embodiment of the present invention relates to treating or lessening the severity of one or more diseases in which STAT6 or a mutant thereof, are known to play a role.

STAT6 functions as a transcription factor to induce gene expression and plays an important role in the IL-4/IL-13 signaling pathway and thus is critical in IL-4/IL-13 mediated biological responses including in human malignancies (e.g., Patel, B. K. R. et al. “Localization of the human stat6 gene to chromosome 12q13. 3-q14. 1, a region implicated in multiple solid tumors.” Genomics 52.2 (1998): 192-200). The STAT6-mediated signaling pathway has been shown to be required for the development of T-helper type 2 (Th2) cells and Th2 immune response and plays a critical role in Th2 lung inflamatory responses including clearance of parasitic infections and in the pathogenesis of asthma (e.g., Walford, H. H, and Doherty, T. A. “STAT6 and lung inflammation.” Jaka-stat 2.4 (2013): e25301). It has been found that STAT6 induces the expression of BCL2L1/BCL-X(L), which is responsible for the anti-apoptotic activity of IL-4 and is shown to play a prominent role in adaptive immunity such as providing innate immune signaling in response to virus infection (e.g., Chen, H., et al. “Activation of STAT6 by STING is critical for antiviral innate immunity.” Cell 147.2 (2011): 436-446). Knockout studies in mice have suggested the role STAT6 in differentiation of T helper 2 (Th2), expression of cell surface markers, and class switch of immunoglobulins. Activation of STAT6 signaling pathway is necessary in tumor-associated macrophages (TAMs) and is implicated in the treatment of cancers and atherosclerosis (e.g., Binnemars-Postma, K., et al. “Targeting the Stat6 pathway in tumor-associated macrophages reduces tumor growth and metastatic niche formation in breast cancer.” The FASEB Journal 32.2 (2018): 969-978; Gong, M., et al. “STAT6 upregulation promotes M2 macrophage polarization to suppress atherosclerosis.” Medical science monitor basic research 23 (2017): 240). STAT6 protein also regulates other transcription factor as Gata3, which is important regulator of Th2 differentiation. STAT6 is also required for the development of IL-9-secreting T cells. STAT6 is also involved in IL4 signaling in B cells, and STAT6 determines the levels of CD20 on the surface of normal and malignant B lymphocytes (e.g., Sandova, V., et al. “IL4-STAT6 signaling induces CD20 in chronic lymphocytic leukemia and this axis is repressed by PI3Kδ inhibitor idelalisib.” haematologica 106.11 (2021): 2995).

In some embodiments, biomarkers associated with the IL-4/13 pathway include IgE, Thymus and activation regulated chemokine (TARC), CD23, periostin, and eosinophils. TARC is a serum TH2 biomarker and chemoattractant for TH2 cell. CD23 is a B cell activation marker and correlates with IgE class switch. Periostin is a serum TH2 biomarker and ECM protein associated with tissue remodeling in atopic diseases.

In some embodiments, treatment with provided a compound results in lesser IL-4 induced TARC release compared to a reference or standard level. In some embodiments, treatment with provided a compound results in lesser IL-13 induced CD23 expression compared to a reference or standard level. In some embodiments, treatment with provided a compound results in lesser IL-13 induced periostin release compared to a reference or standard level.

In some embodiments, treatment with provided a compound inhibits IL-4 induced TARC release. In some embodiments, treatment with provided a compound inhibits IL-13 induced CD23 expression. ISE, treatment with provided a compound inhibits IL-13 induced periostin release.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer, cardiovascular disease, viral disease, autoimmune diseases, autoinflammatory syndromes, atherosclerosis, psoriasis, allergic disorders, inflammatory bowel disease, inflammation, acute and chronic gout and gouty arthritis, neurological disorders, metabolic syndrome, immunodeficiency disorders such as AIDS and HIV, destructive bone disorders, osteoarthritis, proliferative disorders, infectious diseases, conditions associated with cell death, pathologic immune conditions involving T cell activation, and CNS disorders in a patient. In one embodiment, a human patient is treated with a compound of the current invention and a pharmaceutically acceptable carrier, adjuvant, or vehicle, wherein said compound is present in an amount to measurably degrade and/or inhibit STAT6 or a mutant thereof

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma exacerbated or induced following bacterial or viral infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics.

Another aspect of the present invention relates to a method of treating an allergic or inflammatory disease in a subject comprising administering to the subject a therapeutically effective amount of a compound of the present invention to the subject. The disease may be a lung disease such as, e.g., asthma, airway hyperresponsiveness (AHR), an allergic disease, allergic rhinitis, emphysema, chronic obstructive pulmonary disease (COPD), reactive airway disease, chronic rhinosinusitis, or essentially any other disease of the upper or lower airways that produces airflow obstruction.

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g., between the hours of about 4 to 6 am, i.e., at a time normally substantially distant form any previously administered symptomatic asthma therapy.

In some embodiments, STAT6, via its Src homology 2 (SH2) domain, is recruited to the phosphotyrosine residues and is phosphorylated on Tyr641. In some embodiments, STAT6 then dimerizes via reciprocal SH2 domain-pTyr641 interactions, translocates to the nucleus, and participates in the expression of genes leading to asthma and airway hyperresponsiveness (AHR).

In some embodiments, the present invention provides a method of treating asthma in a patient in need thereof, comprising administering a compound of the present invention, or a pharmaceutically acceptable salt thereof.

In some embodiments, the present invention provides a method of treating airway hyperresponsiveness (AHR) in a patient in need thereof, comprising administering a compound of the present invention, or a pharmaceutically acceptable salt thereof.

In some embodiments, the present invention provides a method of treating allergic rhinitis in a patient in need thereof, comprising administering a compound of the present invention, or a pharmaceutically acceptable salt thereof.

In some embodiments, the present invention provides a method of treating allergic asthma in a patient in need thereof, comprising administering a compound of the present invention, or a pharmaceutically acceptable salt thereof.

In some embodiments, the present invention provides a method of treating emphysema in a patient in need thereof, comprising administering a compound of the present invention, or a pharmaceutically acceptable salt thereof.

In some embodiments, the present invention provides a method of treating chronic rhinosinusitis (e.g., with nasal polyposis) in a patient in need thereof, comprising administering a compound of the present invention, or a pharmaceutically acceptable salt thereof.

In some embodiments, the present invention provides a method of treating atopic dermatitis in a patient in need thereof, comprising administering a compound of the present invention, or a pharmaceutically acceptable salt thereof.

In some embodiments, the present invention provides a method of treating COPD in a patient in need thereof, comprising administering a compound of the present invention, or a pharmaceutically acceptable salt thereof.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g., eosinophilia, in particular eosinophil related disorders of the airways (e.g., involving morbid eosinophilic infiltration of pulmonary tissues) including hypereosinophilia as it effects the airways and/or lungs as well as, for example, eosinophil- related disorders of the airways consequential or concomitant to Loffler's syndrome, eosinophilic pneumonia, parasitic (in particular metazoan) infestation (including tropical eosinophilia), bronchopulmonary aspergillosis, esophagitis, polyarteritis nodosa (including Churg-Strauss syndrome), eosinophilic granuloma and eosinophil-related disorders affecting the airways occasioned by drug-reaction.

In some embodiments, the present invention provides a method of treating eosinophilic esophagitis in a patient in need thereof, comprising administering a compound of the present invention, or a pharmaceutically acceptable salt thereof.

In some embodiments, the present invention provides a method of treating prurigo nodularis in a patient in need thereof, comprising administering a compound of the present invention, or a pharmaceutically acceptable salt thereof.

Compounds of the invention are also useful in the treatment of inflammatory or allergic conditions of the skin. In some embodiments, the present invention provides a method of treating inflammatory or allergic conditions of the skin in a patient in need thereof, comprising administering a compound of the present invention, or a pharmaceutically acceptable salt thereof.

In some embodiments the inflammatory disease of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis or primary biliary cholangitis, uveitis (anterior and posterior), Sjogren's syndrome, keratoconjunctivitis sicca and vernal keratoconjunctivitis, interstitial lung disease or fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospirosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus. Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, prurigo nodularis, encephalitis, encephalomyelitis, endocarditis, endometritis, enteritis, enterocolitis, epicondylitis, epididymitis, fasciitis, fibrositis, gastritis, gastroenteritis. Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, the present invention provides a method of treating an autoimmune disease selected from encephalomyelitis, systemic sclerosis, idiopathic pulmonary fibrosis (IPF), inflammatory bowel disease, atopic dermatitis, rheumatoid arthritis, graft versus host disease (acute and chronic), and other tissue fibrosis diseases.

In some embodiments, the present invention provides a method of treating idiopathic interstitial pneumonia(s) (IIPs), including any type of lung fibrosis, either interstitial lung disease associated with rheumatic disease (including SSc) or IPF itself, in a patient in need thereof, comprising administering a compound of the present invention, or a pharmaceutically acceptable salt thereof.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, systemic juvenile idiopathic arthritis (SJIA), cryopyrin associated periodic syndrome (CAPS), and osteoarthritis.

In some embodiments the inflammatory disease which can be treated according to the methods of this invention is a TH17 mediated disease or TH17-associated disease. In some embodiments the TH17 mediated disease or TH17-associated disease is selected from psoriasis, psoriatic arthritis, systemic lupus erythematosus, multiple sclerosis, and inflammatory bowel disease (including Crohn's disease or ulcerative colitis), or graft-versus-host disease.

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

In some embodiments, the present invention provides a method of treating an autoimmune disease or inflammatory disorder is selected from nonalcoholic fatty liver disease (NAFLD), nonalcoholic steatohepatitis (NASH), idiopathic autoimmune hepatitis, progressive fibrosis associated interstitial lung disease, pulmonary arterial hypertension (PAH), immunoglobulin G4-related disease (IgG4-RD), chronic organ rejection (e.g., lung transplant), vasculitides (e.g., vasculitides), and STAT6 gain of function (GOF) mutations.

In some embodiments, the present invention provides a method of treating STAT6 gain of function (GOF) mutations in a patient in need thereof, comprising administering a compound of the present invention, or a pharmaceutically acceptable salt thereof. In some embodiments, the STAT6 GOF mutation is STAT6VT.

In some embodiments, the cardiovascular disease which can be treated according to the methods of the present invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of the present invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV or COVID19 infection.

In some embodiments, the aberrant activation of STAT6 which can be treated according to the methods of this invention is a human cancer. In some embodiments, the human cancer which can be treated according to the methods of this invention include benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas. Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the present invention provides a method of treating a cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes.

In some embodiments, the present invention provides a method of treating a JAK-associated disease. In some embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

In some embodiments, the present invention provides a method of treating a hematologic malignancy selected from LGL leukemia (T and NK cell), cutaneous T cell lymphoma (CTCL), peripheral T cell lymphomas (PTCL, all subtypes including ALCL), diffuse large B cell lymphoma (DLBCL), acute myelogenous leukemia, multiple myeloma, and myelofibrosis.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinirole, pramipexole, bromocriptine, pergolide, trihexyphenidyl, and amantadine; agents for treating Multiple Sclerosis (MS) such as beta interferon (e.g., Avonex® and Rebif®), glatiramer acetate (Copaxone®), and mitoxantrone; treatments for asthma such as albuterol and Singulair©, agents for treating schizophrenia such as zyprexa, risperdal, seroquel, and haloperidol; anti-inflammatory agents such as corticosteroids, TNF blockers, IL-1 R^{A}, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine: neurotrophic factors such as acetylcholinesterase inhibitors, MAO inhibitors, interferons, anti-convulsants, ion channel blockers, riluzole, and anti-Parkinsonian agents: agents for treating cardiovascular disease such as beta-blockers. ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokonazole and ritonavir), pirfenidone (Esbriet®), nintedanib (Ofev®), intravenous immunoglobulins, bosentan (Tracleer®), nifedipine (Procardia XL®), sildenafil (Revatio®), losartan (Cozaar®), iloprost (Ventavis®), topical nitroglycerin, N-acetylcysteine, antiacid therapy, and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphanide (Cytoxan®), chloranbucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvise® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salneterol xinafoate (Serevent®) and fonnoterol (Foradil®), anticholinergic agents such as ipratropiun bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort)), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor(®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexanethasone (Decadron®) in combination with lenalidomide (Revlinid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and fonnoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-Bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and fonnoterol (Foradil®), anticholinergic agents such as ipratropium bromide (Atrovent®) and tiotropium (Spiriva®), methylxanthines such as theophylline (Theo-Dur®, Theolair®, Slo-Bid®, Uniphyl®, Theo-249) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar® Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzcon®) and maraviroc (Sclzcntry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristinc (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenstrom's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca): rucaparib (Rubraca®, Clovis Oncology); niraparib (Zejulak, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333): and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis): abemaciclib (Ly2835219, Eli Lilly): and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081): IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/Biogenlde®); of atinumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER², Herceptin®, Genentech): ado-trastuzumab emtansine (anti-HER2, fused to emtansine. Kadcyla®, Genentech); and pertuzumab (anti-HER2, Perjeta®, Genentech): and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®. GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE, gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors: MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies: compounds which target, decrease or inhibit the activity of Flt-3: Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Confonna Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere® J, Sanofi-Avcntis; Doccfrcz®, Sun Pharmaceutical), albumin-bound paclitaxel (Abraxanc®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly: Vincasar®, Teva Pharmaceuticals: Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb: Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic. Ixempra®, Bristol-Myers Squibb); nelarabine (prodrug of deoxyguanosine analog, nucleoside metabolic inhibitor, Arranon, Novartis); clofarabine (prodrug of ribonucleotide reductase inhibitor, competitive inhibitor of deoxycytidine, Clolar(®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer): and lenvatinib (Lenvima®, Eisai); Raf inhibitors, such as sorafenib (Nexavar®. Bayer AG and Onyx); dabrafenib (Tafinlar®, Novartis); and vemurafenib (Zelboraf®, Genentech/Roche); MEK inhibitors, such as cobimetinib (Cotellic®), Exelexis/Genentech/Roche): trametinib (Mekinist®, Novartis); Bcr-Abl tyrosine kinase inhibitors, such as imatinib (Gleevec®, Novartis); nilotinib (Tasigna(®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®). Novartis); afatinib (Gilotrif®, Boehringer Ingelheim): osimertinib (targeting activated EGFR, Tagrissok, AstraZeneca); and brigatinib (Alunbrig®, Ariad Pharmaceuticals): c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche): Bruton's tyrosine kinase inhibitors, such as ibrutinib (Imbruvicak, Pharmacyclics/Janssen): and Flt3 receptor inhibitors, such as midostaurin (Rydaptk, Novartis).

In some embodiments, the present invention provides a method of treating EGFR-mutant NSCLC in a patient in need thereof, comprising administering a compound of the present invention or a pharmaceutically acceptable salt thereof and one or more EGFR kinase inhibitors (e.g., gefitinib, erlotinib, lapatinib, afatinib, osimertinib, brigatinib, etc.).

In some embodiments, the present invention provides a method of treating EGFR-mutant NSCLC in a patient in need thereof, comprising administering a compound of the present invention or a pharmaceutically acceptable salt thereof and erlotinib.

Other kinase inhibitors and VEGF-R antagonists that are in development and may be used in the present invention include tivozanib (Aveo Pharmaceuticals); vatalanib (Bayer/Novartis): lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547.632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs, host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis. Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Beheet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type 1 hypersensitivity, allergic conjunctivitis, allergic rhinitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints leading to joint inflammation and pain, cartilage and/or bone destruction, as well as bone regrowth and fusion, including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder. (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, idiopathic autoimmune hepatitis, gastritis, enteritis, dermatitis, gingivitis, appendicitis, pancreatitis, cholecystitis, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive or overgrowing bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas. (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced or exacerbated following bacterial or viral infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleredoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung disease or fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer): duvelisib (formerly IPI-145, Infinity Pharmaceuticals); PQR309 (Piqur Therapeutics, Switzerland); and TGR1202 (formerly RP5230, TG Therapeutics).

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors: topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers: antiproliferative antibodies; heparanase inhibitors: inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaniinoethylamino-17-demethoxy-geldanamycin, NSC707545), IPI-504, CNF1010, CNF2024. CNF1010 from Conformia Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastrazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Fannorubicin™. Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfanide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed. e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof: see WO2008118802), navitoclax (and analogs thereof, see U.S. Pat. No. 7,390,799). NH-1 (Shenayng Pharmaceutical University), obatoclax (and analogs thereof, see WO2004106328), S-001 (Gloria Pharmaceuticals), TW series compounds (Univ, of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity: or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR): c) compounds targeting, decreasing or inhibiting the activity of the insulin-like growth factor receptor 1 (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase: g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; 1) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970: AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDKI, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; ilmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213: Tyrphostin AG 1748: Tyrphostin AG 490: Tyrphostin B44: Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180: trastuzumab (Herceptin™), cetuximab (Erbitux™), Iressa, Tarceva, OSI-774, C₁-1033, EKB-569, GW-2016, E11, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAKT/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and: and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvon; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Cclebrcx™), rofecoxib (Vioxx™), etoricoxib, valdecoxib or a 5-alkyl-2- arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™ Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to. PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™); carfilzomib (Kyprolis, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan©), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myceloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]- amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2- propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol, 1, pp, 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™: Endostatin™ anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g. anecortave, triamcinolone, hydrocortisone, 11-α-epihydrocotisol, cortexolone, 17a-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclomethasone dipropionate, fluticasone propionate, ciclesonide or mometasone furoate; non-steroidal glucocorticoid receptor agonists: LTB4 antagonists such LY293111, CGS025019C, CP-195543, SC-53228, BIIL 284, ONO 4057, SB 209247: LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering- Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vemalis), T-440 (Tanabe), KW-4490 (Kyowa Hakko Kogyo): A2a agonists; A2b antagonists: and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratadine, diphenhydramine and fexofenadine hydrochloride, acrivastine, astemizole, azelastine, ebastine, epinastine, mizolastine and terfenadine.

Other useful combinations of compounds of the invention with anti-inflammatory drugs are those with antagonists of chemokine receptors, e.g. CCR-1, CCR-2, CCR-3, CCR-4, CCR-5. CCR-6, CCR-7, CCR-8. CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination.” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1. B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-13B), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TAC. APRIL, BCMA, LTpR, LIGHT, DcR3, HVEM, VEGI/TLLA, TRAMP/DR3, EDAR, EDA1, XEDAR, EDA2, TNFR1, Lymphotoxin α/TNFβ, TNFR2, TNFa, LTβR, Lymphotoxin α1β2, FAS, FASL, RELT, DR⁶, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69. Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR³ and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fe portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleanine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb): Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intetumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol, 14, 1212-1218; Zou et al. (2016) Sci. Transl. Med, 8. The anti-PD-1 antibody nivolumab (Opdivo*, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelarcorep (Rcolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCTO1619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CGO070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations: and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody: lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORyt.

In some embodiments, an immunostimulatory therapeutic is recombinant human interleukin 15 (rhIL-15), rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15. Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those described in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018. Vol, 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTER) antibody constructs can mediate bystander tumor cell killing”. PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4. CD160, CGEN-15049. CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2. B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody). BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pcmbrolizumab (anti-PD-1 antibody. Keytruda®, Merck); ipilimumab (anti-CTLA-4 antibody, Yervoy®, Bristol-Myers Squibb); durvalumab (anti-PD-L1 antibody, Imfinzi®, AstraZeneca): and atezolizumab (anti-PD-L1 antibody, Tecentriq®. Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®). Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed): and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MED10562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCTO1303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566. Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127. Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134): and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX⁵¹⁸ (Leap Therapeutics), an agonistic anti-GITR antibody, in malignant melanoma and other malignant solid tumors (NCT01239134 and NCT02628574): GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDT1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387. NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include AL^{X}-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176): and ADU-S100 (MIW815, Aduro Biotech/Novartis), an agonistic synthetic cyclic dinucleotide, in Phase 1 (NCT02675439 and NCT03172936).

In some embodiments, STAT6 inhibition/degradation can significantly enhance CDN-induced STING signaling and antitumor immunity (Pei et al., Can. Lett, 2019, 450:110).

Checkpoint inhibitors that may be used in the present invention include CSF1R inhibitors. CSF1R inhibitors that are being studied in clinical trials include pexidartinib (PL^{X} 3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSFIR, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201. Innate Pharma), an anti-NKG2A antibody, in head and neck neoplasms (NCT02643550) and chronic lyinphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed, 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, |
| | GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TEA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Fonnic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH3·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

In some instances, intermediates and compounds described in the examples comprise one or more stereocenters and more than one enantiomer/diastereomer was produced. In some embodiments, these enantiomers/diastereomers were separated and isolated, although stereochemistry was not resolved. Unless otherwise stated, stereochemistry was assigned arbitrarily. For intermediates, each enantiomer/diastereomer with arbitrarily assigned stereochemistry may result in a final compound (e.g., assigned a “I—” number), which also maintains the arbitrarily assigned stereochemistry. Accordingly, any compound with arbitrarily assigned stereochemistry or produced from an intermediate with arbitrarily assigned stereochemistry may be depicted herein as a certain stereoisomer, but it is understood that such compound may be the other stereoisomer (i.e., enantiomer or diastereomer).

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

TABLE 3
Compounds synthesized via the reductive amination of the amines and aldehydes

| | LCMS | |
|---|---|---|
| | (ESI+) m/z | |
| I-# | (M + H)+ | ¹H NMR (400 MHz, DMSO-d₆) δ |
| I-1292 | 902.4 | 11.82 (d, J = 7.6 Hz, 1H), 11.10 (s, 1H), 8.09 (d, J = 15.6 Hz, 1H), 7.69 (d, J = |
| | | 5.6 Hz, 1H), 7.15 (d, J = 8.4 Hz, 1H), 6.99 (d, J = 5.2 Hz, 2H), 6.96-6.93 |
| | | (m, 1H), 6.86 (dd, J = 6.0, 17.6 Hz, 1H), 6.67 (s, 1H), 6.60 (d, J = 8.4 Hz, |
| | | 1H), 6.46 (s, 1H), 5.38 (dd, J = 5.4, 12.8 Hz, 1H), 4.63 (t, J = 6.8 Hz, 2H), |
| | | 4.08-3.90 (m, 1H), 3.80-3.72 (m, 1H), 3.70 (d, J = 4.8 Hz, 3H), 3.62 (s, |
| | | 3H), 3.29-3.22 (m, 4H), 3.20-3.09 (m, 9H), 3.06-3.00 (m, 3H), 2.88-2.79 |
| | | (m, 1H), 2.75-2.63 (m, 8H), 2.04-1.97 (m, 1H), 1.97-1.79 (m, 4H), 1.79- |
| | | 1.58 (m, 2H), 1.40-1.27 (m, 1H) |

^{a}The reductive amination was run under standard techniques anywhere from 0-40° C. for 1-3 hrs. Other bases, such as AcONa, and reducing agents, such as NaBH(Oac)₃, could be employed. 4A molecular sieves also could be added to promote the reaction when applicable. Final compounds were purified via standard techniques including prep-HPLC and other chromatography techniques. Standard deprotection conditions such as HCl/dioxane or TFA/DCM could also be employed to deprotect final products when applicable.

TABLE 4
Compounds synthesized via the cross coupling of the chlorides and boronic acids

| | LCMS | |
|---|---|---|
| | (ESI+) m/z | |
| I-# | (M + H)+ | ¹H NMR (400 MHz, DMSO-d₆) δ |
| I- | 868.4 | 11.09 (s, 1H), 8.63 (s, 1H), 8.14-8.06 (m, 3H), 7.73-7.66 (m, 1H), 7.62- |
| 1272 | | 7.52 (m, 1H), 7.15 (d, J = 8.4 Hz, 1H), 7.02-6.90 (m, 5H), 6.74 (t, J = 6.0 Hz, |
| | | 1H), 6.63 (s, 1H), 6.58 (dd, J = 1.6, 8.8 Hz, 1H), 6.15-6.06 (m, 1H), 5.37 (dd, |
| | | J = 5.4, 12.4 Hz, 1H), 4.62 (q, J = 6.8 Hz, 2H), 4.35-4.23 (m, 2H), 3.76 (d, J = |
| | | 4.0 Hz, 3H), 3.68-3.56 (m, 5H), 3.24-3.20 (m, 4H), 3.16-3.04 (m, 4H), 2.95- |
| | | 2.84 (m, 1H), 2.74-2.58 (m, 8H), 2.37-2.23 (m, 2H), 2.05-1.96 (m, 1H) |
| I- | 879.3 | 11.10 (s, 1H), 8.12 (s, 1H), 8.00 (dd, J = 8.4, 16.8 Hz, 1H), 7.97-7.89 (m, |
| 1293 | | 1H), 7.70 (d, J = 4.0 Hz, 1H), 7.51 (dd, J = 1.6, 11.6 Hz, 1H), 7.47 (s, 1H), |
| | | 7.43 (s, 1H), 7.09 (d, J = 8.4 Hz, 1H), 6.99 (d, J = 5.6 Hz, 2H), 6.97-6.93 (m, |
| | | 1H), 6.71 (s, 1H), 6.67-6.63 (m, 1H), 6.53 (d, J = 12.8 Hz, 1H), 5.43-5.33 |
| | | (m, 1H), 4.65 (t, J = 6.8 Hz, 2H), 4.49 (d, J = 14.4 Hz, 2H), 3.64-3.58 (m, |
| | | 8H), 3.22-3.11 (m, 5H), 2.98-2.84 (m, 8H), 2.75-2.63 (m, 10H), 2.40-2.35 |
| | | (m, 1H), 2.28-2.09 (m, 1H), 2.05-1.97 (m, 1H) |

^{a}The cross coupling was performed under standard techniques from 30-80° C. for 1-3 hrs. Other catalysts systems could be utilized, such as Pd(dppf)Cl₂, and bases, such as K₃PO₄. Other halides such as bromides could also be utilized. Final compounds were purified via standard techniques including prep-HPLC and other chromatography techniques.

### Example 1. STAT6 Assay Results

### STAT6 HTRF Results

HTRF binding assays were perforned using 0.15 nM biotinylated truncated STAT6 (123-632)-avi purified from E. coli, 1× Streptavidin-terbium (CisBio) prepared by mixing SA-Tb in PPI detection buffer (CisBio), 20 nM proprietary fluorescein-labeled probe, and test compounds in assay buffer consisting of 50 mM HEPES-Na pH 7.5, 100 mM NaCl, 1 mM EDTA, 2 mM DTT, 0.1% Tween-20 with a final volume of 20 uL. Compound stocks were dissolved at 10 mM in 100% DMSO and 11 point titration with 3 fold serial dilution was performed in white, opaque 384 well microplates. Reaction plates were incubated at room temperature for 30 minutes. Plates were centrifuged at low rpm for 5 mins, and the ratio of fluorescence intensities were measured at emission wavelengths for fluorescein acceptor (520 nm) and terbium donor (495 nm) on Envision Plate reader. % Inhibition was calculated from the 520/495 ratio generated by using proprietary positive control compound for 100% inhibition and DMSO only reactions for 0% inhibition. Data was processed and dose response curves were generated using GraphPad Prism to determine the concentration required for inhibiting 50% of the HTRF signal (IC₅₀).

The STAT6 HTRF results are shown in Table 5. The letter codes for IC₅₀ (μM) include: A (<0.1 μM); B (0.1-1 μM); C (>1-10 μM); D (>10-100 μM); E (>100 μM); and F (not tested).

TABLE 5
STAT6 HTRF Results

| | STAT6 |
|---|---|
| | HTRF: |
| | Average |
| I-# | IC₅₀ (μM) |
| I-61 | F |
| I-98 | D |
| I-105 | F |
| I-1270 | C |
| I-1271 | C |
| I-1272 | B |
| I-1282 | D |
| I-1292 | F |
| I-1293 | A |
| I-1302 | C |
| I-1309 | F |
| I-1323 | B |
| I-1324 | D |
| I-1325 | F |
| I-1326 | F |
| I-1332 | F |
| I-1333 | F |
| I-1334 | C |
| I-1336 | C |
| I-1338 | D |
| I-1340 | F |
| I-1342 | F |
| I-1401 | D |
| I-1458 | D |
| I-1459 | D |
| I-1460 | D |
| I-1472 | C |
| I-1473 | D |
| I-1474 | F |
| I-1536 | F |

### Example 2 STAT6 MSD Degradation Results

Degradation of STAT6 in cells was quantitatively measured using Meso Scale Discovery (MSD) technology. A549 cells were seeded in 96-well plates with a density of 1.5×10e4 to 5×10e5 cells per well in 100 μl fresh media. Compounds were then added to the assay plates with a final top concentration of up to 10 uM in a 1:3 dilution series with total of 9 doses. The assay plates were then incubated for 4 to 24 hours at 37° C. under 5% CO₂. The assay plates were then centrifuged for 5 minutes and the cell pellets were treated with 100 μl/well RIPA lysis buffer (Boston Bioproducts, BP-115D) with protease and phosphatase inhibitors (Roche, 05892791001 and 04906837001). To prepare the MSD assay plates (MSD, L15XA), the plates were coated with the capture antibody (Abnova, H00006778) in PBS, at 40 μl/well. The plates were then incubated overnight at 4° C., washed 1 time with 150 U/well TBST buffer (Cell Signaling Technology, #9997S) and blocked with 150 μl/well blocking buffer (MSD, R93BA-4). Cell lysates were then added to MSD assay plates and the plates were incubated at room temperature for 1 hour. The plates were then washed 3 times with 150 μl/well TBST buffer and 25 μl/well primary detection antibody (Cell Signaling Technology, #5397S). The assay plates were then incubated at room temperature for 1 hour, washed 3 times with 150 μl/well TBST buffer, and 25 μl/well secondary detection antibody, SULFO-TAG anti-rabbit antibody (MSD, R32AB-1) were added. The assay plates were then incubated at room temperature for 1 hour, washed 3 times with 150 μl/well TBST buffer, and 150 μl/well MSD reading buffer (MSD, R92TC-2) were added. The plates were then analyzed by an MSD reader. The data was analyzed by GraphPad Prism and the dose dependent STAT6 degradation was fit using the four-parameter inhibitor vs. response nonlinear regression.

The STAT6 MSD degradation results in A549 cells are shown in Table 6. The letter codes for DC₅₀ (nM) include: A (<1 nM); B (1-10 nM); C (>10-100 nM); D (>100-1000 nM); E (>1000 nM); and F (not tested). The letter codes for Dmax (%) include: A (>90%); B (>70-90%); C (>50-70%); D (<50%); and E (not tested).

TABLE 6

| | STAT6 MSD | |
|---|---|---|
| STAT6 | degradation | STAT6 MSD |
| MSD | DC50 (nM) | degradation |
| Degradation | 24 h | Dmax % 24 h |
| I-61 | E | D |
| I-98 | E | D |
| I-105 | E | D |
| I-1270 | F | E |
| I-1271 | F | E |
| I-1272 | F | E |
| I-1282 | F | E |
| I-1292 | F | E |
| I-1293 | F | E |
| I-1302 | F | E |
| I-1309 | F | E |
| I-1323 | F | E |
| I-1324 | F | E |
| I-1325 | F | E |
| I-1326 | F | E |
| I-1332 | F | E |
| I-1333 | F | E |
| I-1334 | F | E |
| I-1336 | F | E |
| I-1338 | F | E |
| I-1340 | F | E |
| I-1342 | F | E |
| I-1401 | F | E |
| I-1458 | F | E |
| I-1459 | F | E |
| I-1460 | F | E |
| I-1472 | F | E |
| I-1473 | F | E |
| I-1474 | F | E |
| I-1536 | F | E |

### Example 3. STAT6 HiBiT Degradation in A549

Degradation of STAT6 in endogenously tagged STAT6-HiBiT A549 cells was quantitatively measured using the Nano-Glo HiBiT Lytic Detection System (Promega, 3040). A549 (STAT6-HiBiT) cells were seeded in 384-well plates with a density of 1.25k cells per well in 20 μl fresh media. Compounds were then added to the assay plates using the Apricot S3 with a final top concentration of up to 10 uM in a 4-fold dilution series with total of 10 doses. The assay plates were then incubated for 24 hours at 37° C. under 5% CO₂. At the assay endpoint, 20 μL of Nano-Glo HiBiT Lytic Reagent was added to each well and agitated on a plate shaker at 300 rpm for 10 minutes at room temperature. Luminescence was read on PHERAStar FSX Instrument (Is integration time). The data was analyzed by GraphPad Prism and the dose dependent STAT6 degradation was fit using the four-parameter inhibitor vs. response nonlinear regression.

The STAT6 HiBiT degradation results in A549 cells are shown in Table 7. The letter codes for DC₅₀ (nM) include: A (<1 nM): B (1-10 nM); C (>10-100 nM); D (>100-1000 nM); E (>1000 nM); and F (not tested). The letter codes for Dmax (%) include: A (>90%); B (>70-90%); C (>50-70%); D (<50%): and E (not tested).

TABLE 7
STAT6 HiBiT Degradation Results

| | STAT6 | |
|---|---|---|
| | HiBiT | |
| | degradation | STAT6 HiBiT |
| | DC50 (nM) | degradation |
| I-# | 24 h | Dmax % 24 h |
| I-61 | F | E |
| I-98 | F | E |
| I-105 | F | E |
| I-1270 | E | D |
| I-1271 | E | D |
| I-1272 | A | A |
| I-1282 | E | D |
| I-1292 | A | A |
| I-1293 | A | A |
| I-1302 | B | B |
| I-1309 | E | D |
| I-1323 | D | B |
| I-1324 | E | D |
| I-1325 | F | E |
| I-1326 | F | E |
| I-1332 | F | E |
| I-1333 | F | E |
| I-1334 | C | B |
| I-1336 | E | D |
| I-1338 | E | D |
| I-1340 | F | E |
| I-1342 | F | E |
| I-1401 | F | E |
| I-1458 | F | E |
| I-1459 | F | E |
| I-1460 | F | E |
| I-1472 | F | E |
| I-1473 | F | E |
| I-1474 | F | E |
| I-1536 | F | E |

1-31. (canceled)

32. A compound, wherein said compound is selected from:

| I- | Structure |
|---|---|
| 61 | 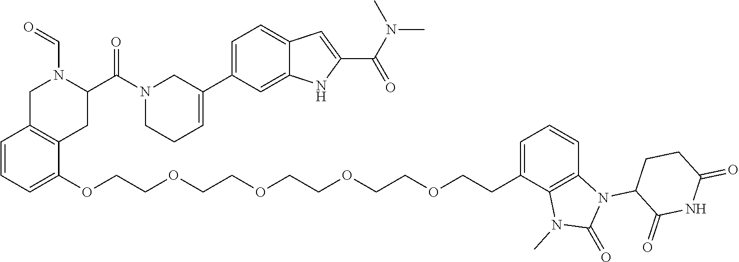 [SMILES: CN(C)C(=O)c1cc2ccc(C3=CCCN(C(=O)C4Cc5c(cccc5OCCOCCOCCOCCOCCc5cccc6c5n(C)c(=O)n6C5CCC(=O)NC5=O)CN4C=O)C3)cc2[nH]1] |
| 98 | 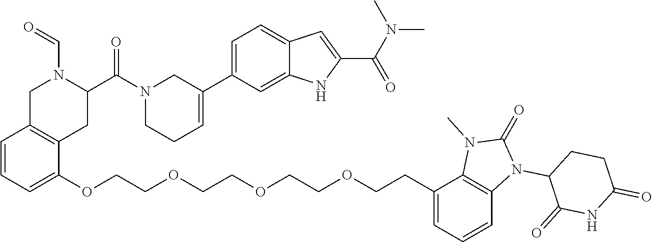 [SMILES: CN(C)C(=O)c1cc2ccc(C3=CCCN(C(=O)C4Cc5c(cccc5OCCOCCOCCOCCc5cccc6c5n(C)c(=O)n6C5CCC(=O)NC5=O)CN4C=O)C3)cc2[nH]1] |
| 105 | 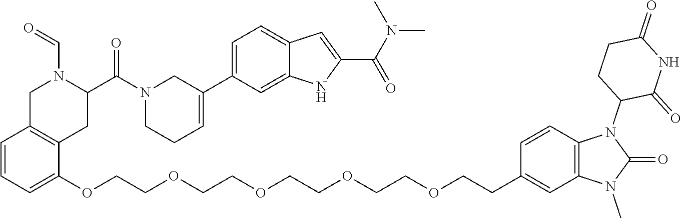 [SMILES: CN(C)C(=O)c1cc2ccc(C3=CCCN(C(=O)C4Cc5c(cccc5OCCOCCOCCOCCOCCc5ccc6c(c5)n(C)c(=O)n6C5CCC(=O)NC5=O)CN4C=O)C3)cc2[nH]1] |
| 1270 | 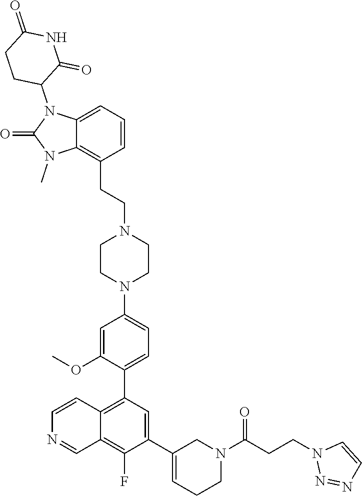 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2cnccc12] |
| 1271 | 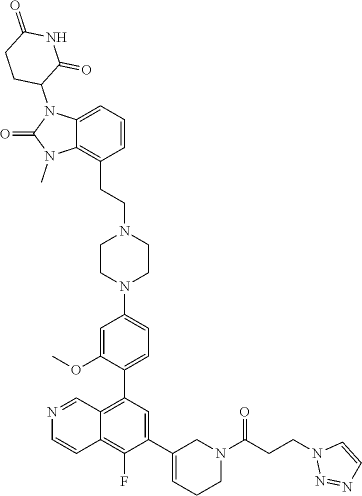 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2ccncc12] |
| 1272 | 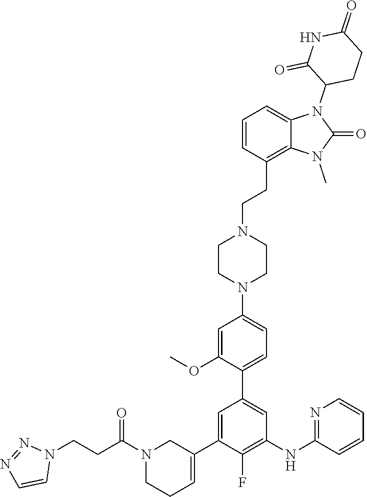 [SMILES: COc1cc(N2CCN(CCc3cccc4c3n(C)c(=O)n4C3CCC(=O)NC3=O)CC2)ccc1-c1cc(Nc2ccccn2)c(F)c(C2=CCCN(C(=O)CCn3ccnn3)C2)c1] |
| 1282 | 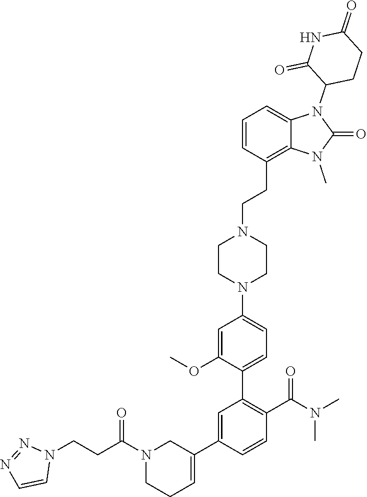 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1C(=O)N(C)C] |
| 1292 | 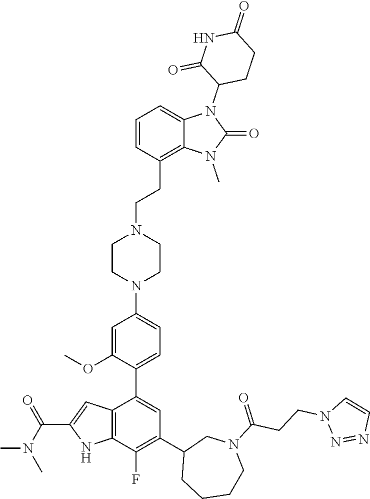 [SMILES: COc1cc(N2CCN(CCc3cccc4c3n(C)c(=O)n4C3CCC(=O)NC3=O)CC2)ccc1-c1cc(C2CCCCN(C(=O)CCn3ccnn3)C2)c(F)c2[nH]c(C(=O)N(C)C)cc12] |
| 1293 | 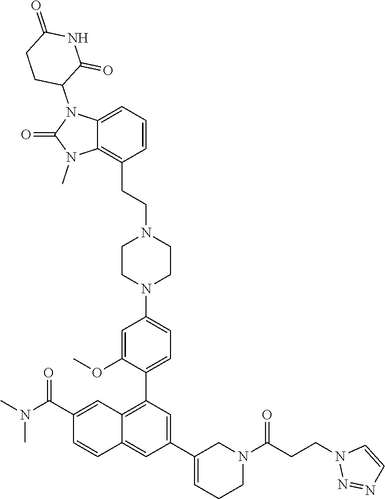 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccc(C(=O)N(C)C)cc12] |
| 1302 | 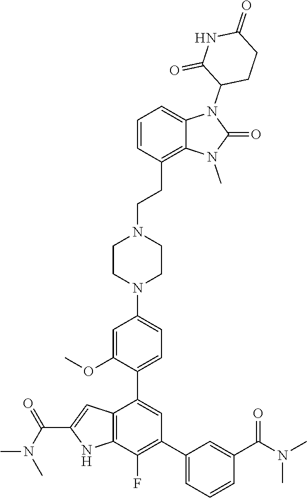 |
| 1309 | 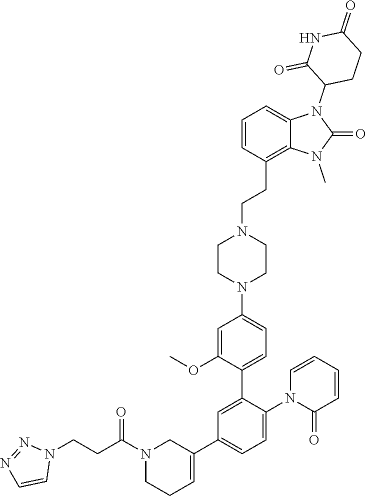 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-n1ccccc1=O] |
| 1323 | 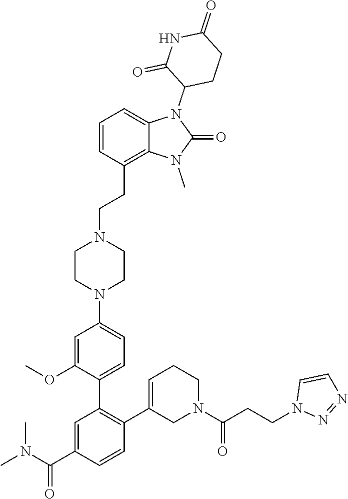 [SMILES: COc1cc(N2CCN(CCc3cccc4c3n(C)c(=O)n4C3CCC(=O)NC3=O)CC2)ccc1-c1cc(C(=O)N(C)C)ccc1C1=CCCN(C(=O)CCn2ccnn2)C1] |
| 1324 | 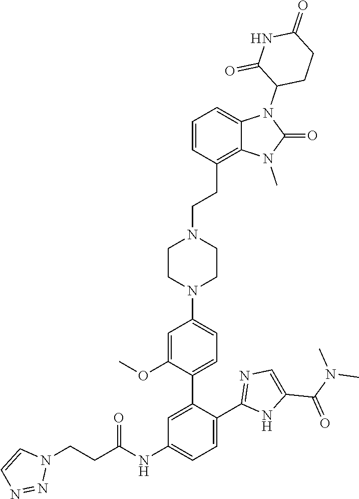 [SMILES: COc1cc(N2CCN(CCc3cccc4c3n(C)c(=O)n4C3CCC(=O)NC3=O)CC2)ccc1-c1cc(NC(=O)CCn2ccnn2)ccc1-c1ncc(C(=O)N(C)C)[nH]1] |
| 1325 | 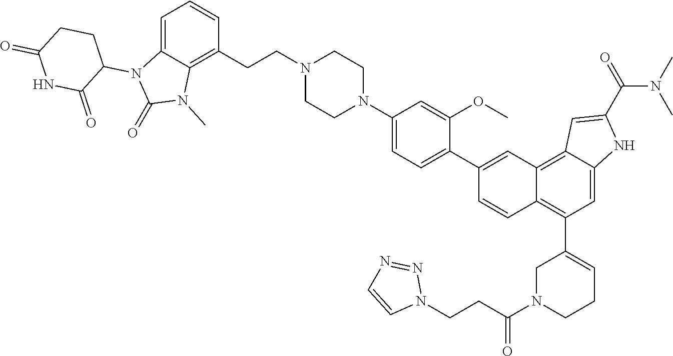 [SMILES: COc1cc(N2CCN(CCc3cccc4c3n(C)c(=O)n4C3CCC(=O)NC3=O)CC2)ccc1-c1ccc2c(C3=CCCN(C(=O)CCn4ccnn4)C3)cc3[nH]c(C(=O)N(C)C)cc3c2c1] |
| 1326 | 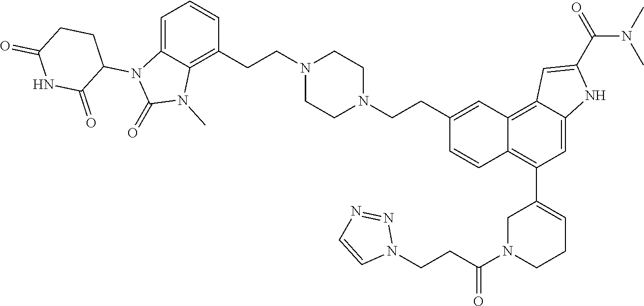 [SMILES: CN(C)C(=O)c1cc2c(cc(C3=CCCN(C(=O)CCn4ccnn4)C3)c3ccc(CCN4CCN(CCc5cccc6c5n(C)c(=O)n6C5CCC(=O)NC5=O)CC4)cc32)[nH]1] |
| 1332 | 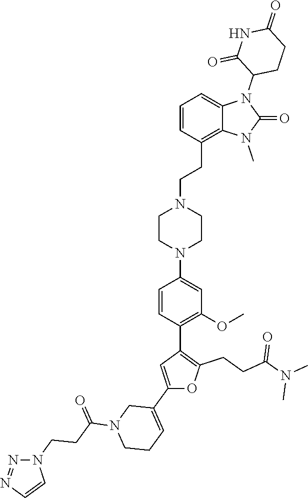 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)oc1CCC(=O)N(C)C] |
| 1333 | 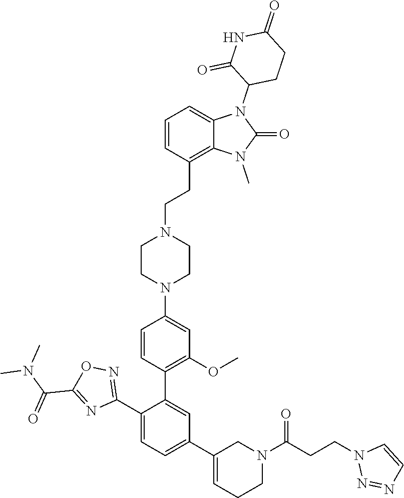 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)ccc1-c1noc(C(=O)N(C)C)n1] |
| 1334 | 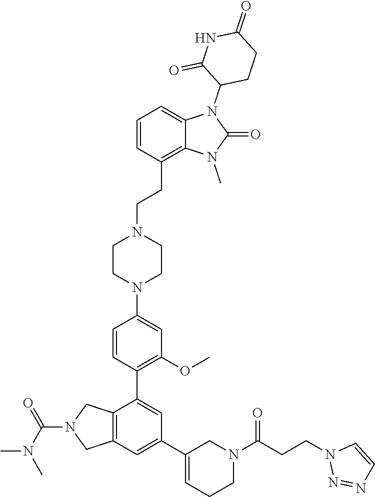 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1CN(C(=O)N(C)C)C2] |
| 1336 | 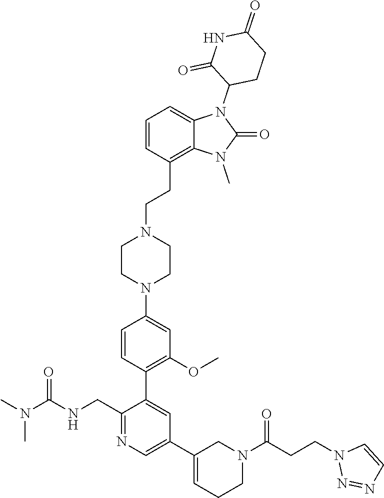 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cnc1CNC(=O)N(C)C] |
| 1338 | 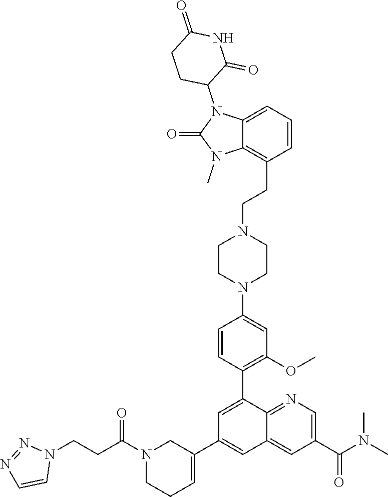 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2cc(C(=O)N(C)C)cnc12] |
| 1340 | 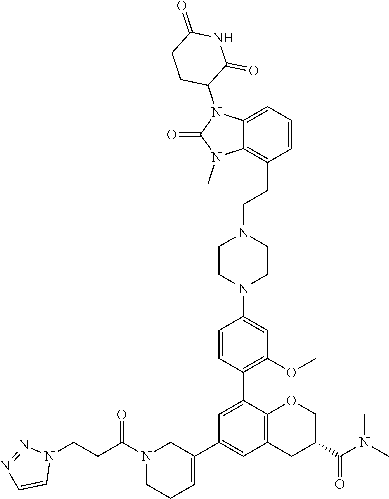 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1OC[C@H](C(=O)N(C)C)C2] |
| 1342 | 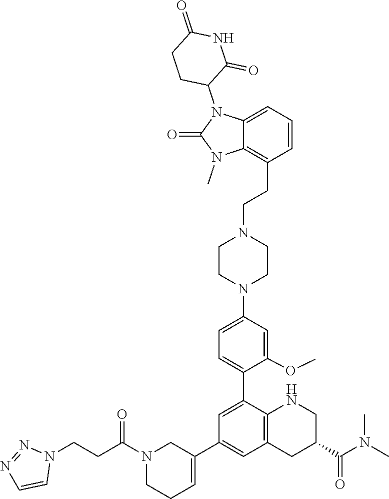 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1NC[C@H](C(=O)N(C)C)C2] |
| 1401 | 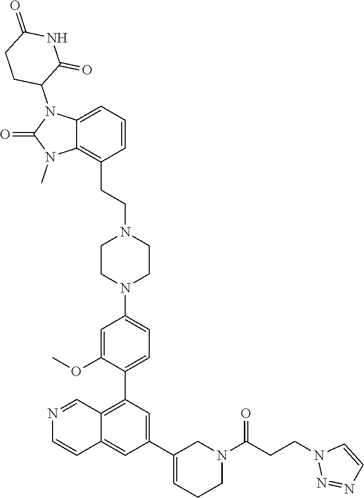 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccncc12] |

or a pharmaceutically acceptable salt thereof.

33. The compound of claim 32, wherein the compound is: 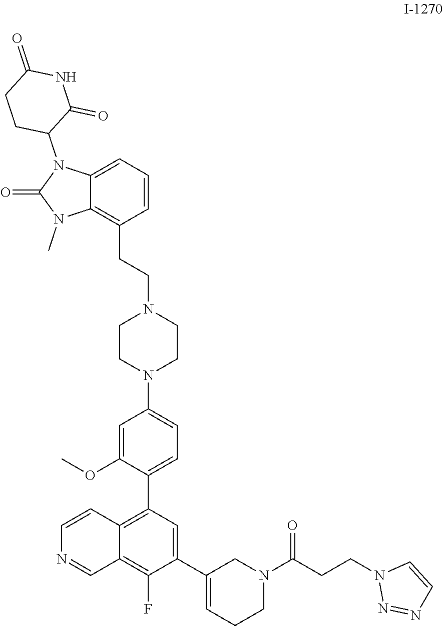 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2cnccc12]
or a pharmaceutically acceptable salt thereof.

34. The compound of claim 32, wherein the compound is: 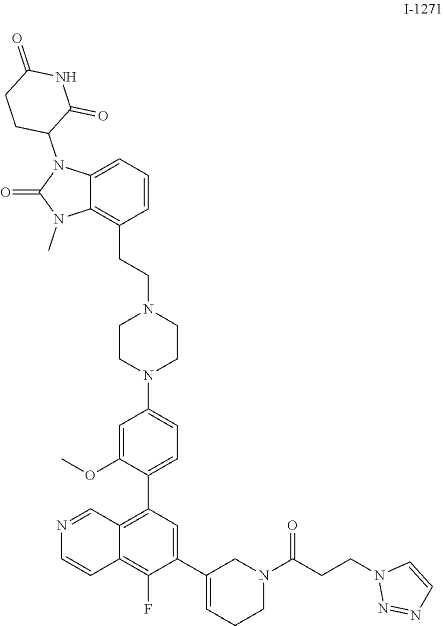 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)c(F)c2ccncc12]
or a pharmaceutically acceptable salt thereof.

35. The compound of claim 32, wherein the compound is:

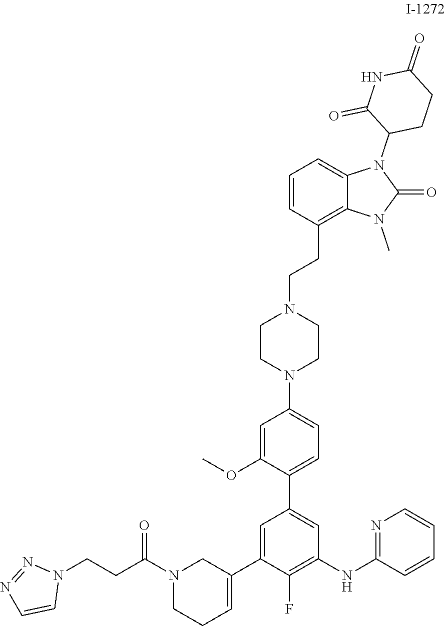 [SMILES: COc1cc(N2CCN(CCc3cccc4c3n(C)c(=O)n4C3CCC(=O)NC3=O)CC2)ccc1-c1cc(Nc2ccccn2)c(F)c(C2=CCCN(C(=O)CCn3ccnn3)C2)c1]

36. The compound of claim 32, wherein the compound is: 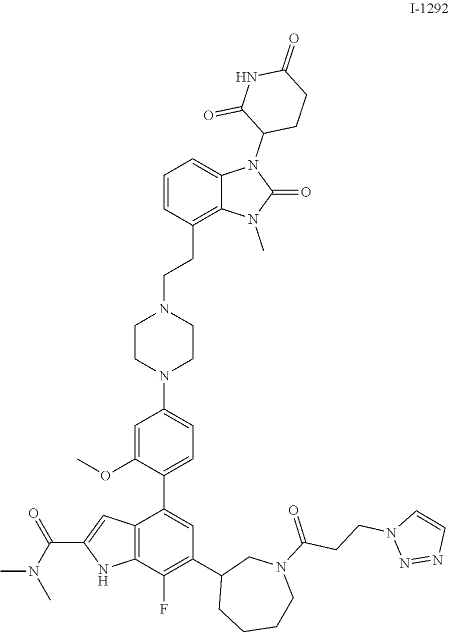 [SMILES: COc1cc(N2CCN(CCc3cccc4c3n(C)c(=O)n4C3CCC(=O)NC3=O)CC2)ccc1-c1cc(C2CCCCN(C(=O)CCn3ccnn3)C2)c(F)c2[nH]c(C(=O)N(C)C)cc12]
or a pharmaceutically acceptable salt thereof;
or a pharmaceutically acceptable salt thereof.

37. The compound of claim 32, wherein the compound is: 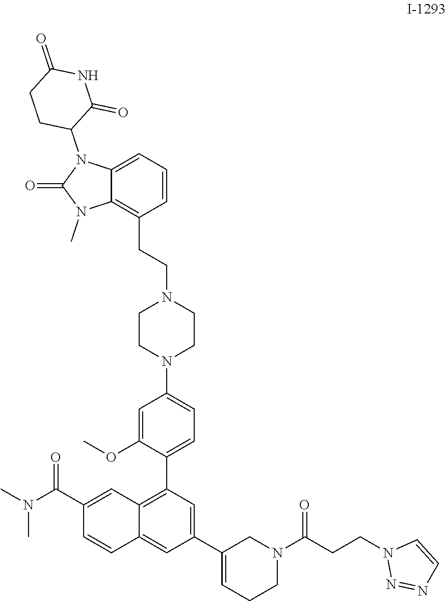 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccc(C(=O)N(C)C)cc12]
or a pharmaceutically acceptable salt thereof.

38. The compound of claim 32, wherein the compound is: 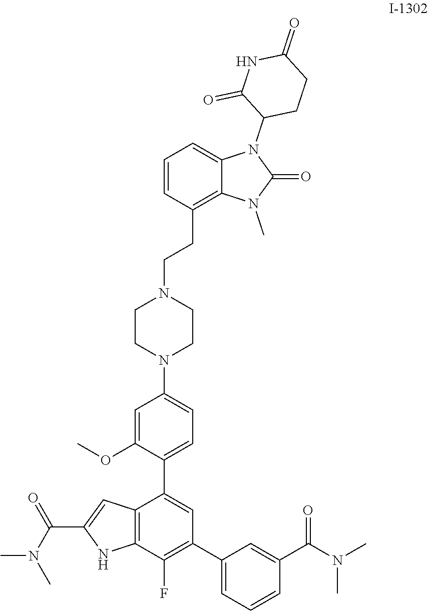
or a pharmaceutically acceptable salt thereof.

39. The compound of claim 32, wherein the compound is: 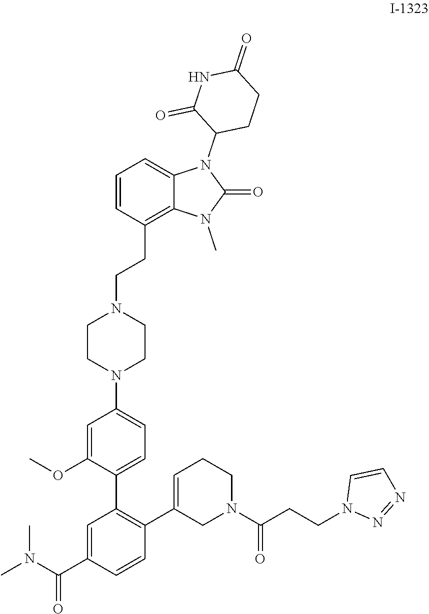 [SMILES: COc1cc(N2CCN(CCc3cccc4c3n(C)c(=O)n4C3CCC(=O)NC3=O)CC2)ccc1-c1cc(C(=O)N(C)C)ccc1C1=CCCN(C(=O)CCn2ccnn2)C1]
or a pharmaceutically acceptable salt thereof.

40. The compound of claim 32, wherein the compound is: 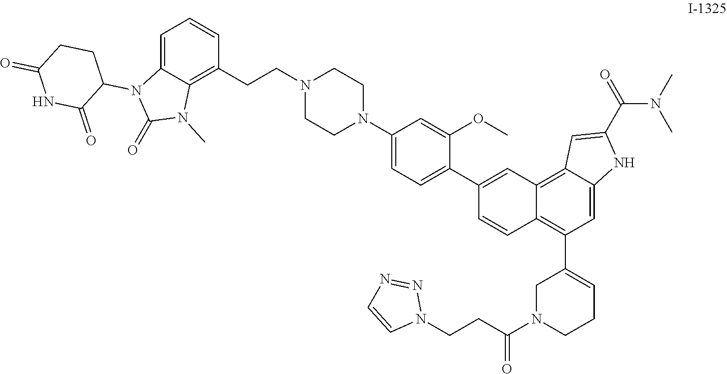 [SMILES: COc1cc(N2CCN(CCc3cccc4c3n(C)c(=O)n4C3CCC(=O)NC3=O)CC2)ccc1-c1ccc2c(C3=CCCN(C(=O)CCn4ccnn4)C3)cc3[nH]c(C(=O)N(C)C)cc3c2c1]
or a pharmaceutically acceptable salt thereof.

41. The compound of claim 32, wherein the compound is: 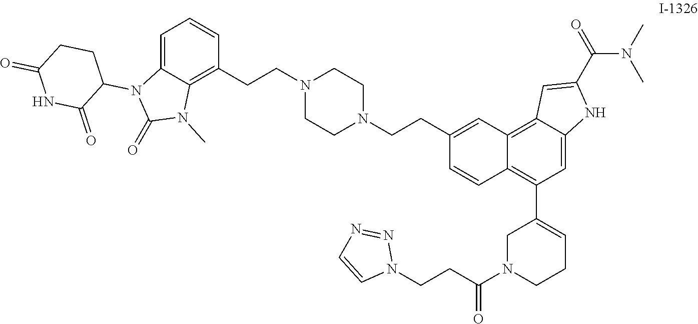 [SMILES: CN(C)C(=O)c1cc2c(cc(C3=CCCN(C(=O)CCn4ccnn4)C3)c3ccc(CCN4CCN(CCc5cccc6c5n(C)c(=O)n6C5CCC(=O)NC5=O)CC4)cc32)[nH]1]
or a pharmaceutically acceptable salt thereof.

42. The compound of claim 32, wherein the compound is: 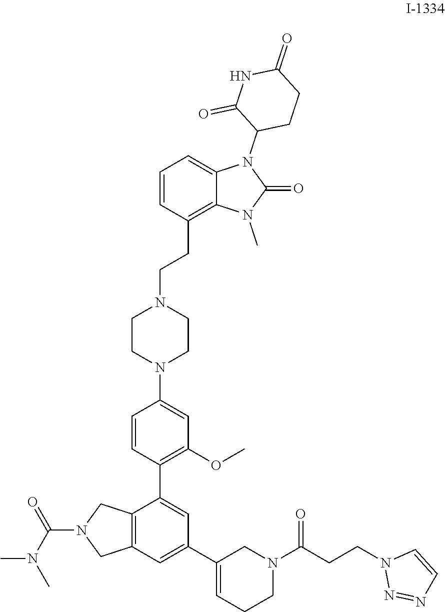 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1CN(C(=O)N(C)C)C2]
or a pharmaceutically acceptable salt thereof.

43. The compound of claim 32, wherein the compound is: 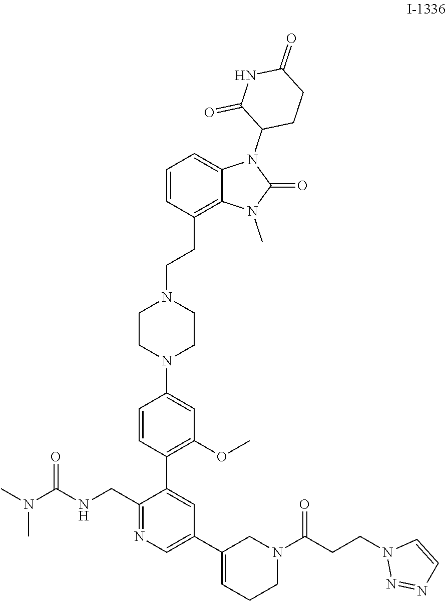 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cnc1CNC(=O)N(C)C]
or a pharmaceutically acceptable salt thereof.

44. The compound of claim 32, wherein the compound is: 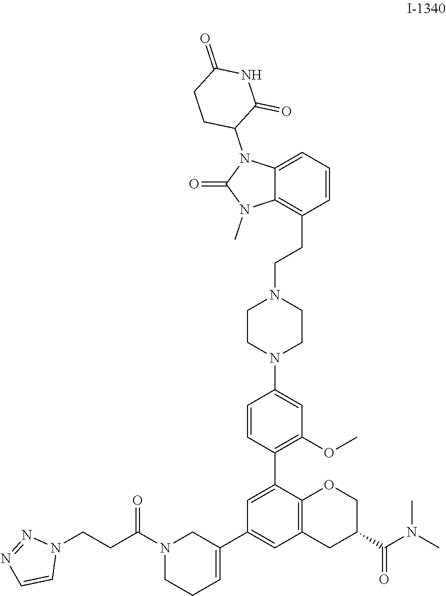 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1OC[C@H](C(=O)N(C)C)C2]
or a pharmaceutically acceptable salt thereof.

45. The compound of claim 32, wherein the compound is: 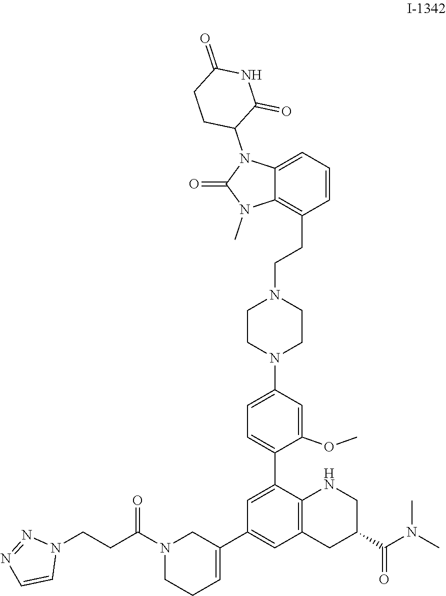 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2c1NC[C@H](C(=O)N(C)C)C2]
or a pharmaceutically acceptable salt thereof.

46. The compound of claim 32, wherein the compound is: 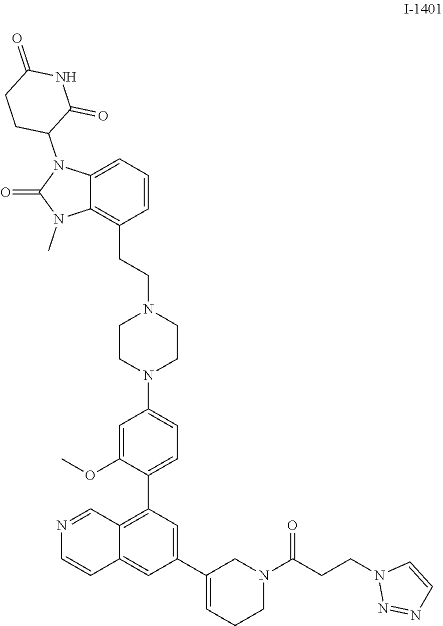 [SMILES: COc1cc(N2CCN(CCc3cccc4c3n(C)c(=O)n4C3CCC(=O)NC3=O)CC2)ccc1-c1cc(C2=CCCN(C(=O)CCn3ccnn3)C2)cc2ccncc12]
or a pharmaceutically acceptable salt thereof.

47. A pharmaceutical composition comprising a compound of claim 32, and a pharmaceutically acceptable carrier, adjuvant, or vehicle.

48. A method of degrading STAT6 in a patient or biological sample comprising administering to said patient, or contacting said biological sample with a compound of claim 32, or a pharmaceutical composition thereof.

49. A method of treating a STAT6-mediated disorder, disease, or condition in a patient comprising administering to said patient a compound of claim 32, or a pharmaceutical composition thereof.

50. The method of claim 49, wherein STAT6-mediated disorder, disease, or condition is cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

51. A method of treating asthma, atopic dermatitis, COPD, chronic rhinosinusitis, chronic rhinosinusitis with nasal polyps, eosinophilic esophagitis, prurigo nodularis, or bullous pemphigoid in a patient comprising administering to said patient a compound of claim 32, or a pharmaceutical composition thereof.
